(19) United States
(12) Patent Application Publication
(10) Pub. No.: US 2023/0117034 A1
(43) Pub. Date: Apr. 20, 2023

(54) PYRAZOLOTRIAZINES

(71) Applicants: Bayer Aktiengesellschaft, Leverkusen (DE); Deutsches Krebsforschungszentrum, Heidelberg (DE)

(72) Inventors: Kai THEDE, Berlin (DE); Philipp BUCHGRABER, Berlin (DE); Gerhard SIEMEISTER, Berlin (DE); Patrick STEIGEMANN, Berlin (DE); Antje Margret WENGNER, Berlin (DE); Ulf BOEMER, Glienicke (DE); Naomi BARAK, Berlin (DE); Philip LIENAU, Berlin (DE)

(73) Assignees: Bayer Aktiengesellschaft, Leverkusen (DE); Deutsches Krebsforschungszentrum, Heidelberg (DE)

(21) Appl. No.: 17/784,545

(22) PCT Filed: Dec. 9, 2020
(86) PCT No.: PCT/EP2020/085299

§ 371 Date: Jun. 10, 2022

(30) Foreign Application Priority Data
Dec. 11, 2019 (EP) 19215354.2
Mar. 6, 2020 (EP) 20161680.2

(51) Int. Cl. A61K 31/53; C07D 487/04; C07D 519/00; A61K 45/06; A61K 31/5377; A61K 31/553; A61K 31/5386; A61K 31/541; A61K 31/551; A61P 35/00; A61P 11/00

(52) U.S. Cl. CPC A61K 31/53; C07D 487/04; C07D 519/00; A61K 45/06; A61K 31/5377; A61K 31/553; A61K 31/5386; A61K 31/541; A61K 31/551; A61P 35/00; A61P 11/00

(57) ABSTRACT

The present invention provides compounds of general formula (I), in which X, R¹, R² and R³ are as described and defined herein, methods of preparing said compounds, intermediate compounds useful for preparing said compounds, pharmaceutical compositions and combinations comprising said compounds, and the use of said compounds for manufacturing pharmaceutical compositions for the treatment and/or prophylaxis of diseases, in particular of hyperproliferative disorders such as cancer disorders, as a sole agent or in combination with other active ingredients.

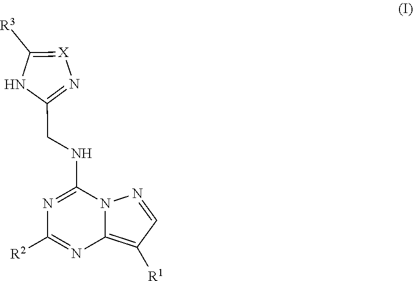

### BACKGROUND

The present invention provides compounds of general formula (I) which impair the activity of CDK12. In particular, the present invention provides compositions and methods for the treatment of cancer and other CDK12-dependant diseases. More particularly, the present invention provides compounds which induce the proteolytic degradation of CDK12 and/or Cyclin K in the cell. Thus, the present invention provides compounds capable of degrading CDK12 and/or Cyclin K for the treatment of breast cancer, liver cancer, lung cancer, ovarian cancer, endometrial cancer, cervical cancer, colorectal cancer, gastric cancer, esophageal cancer, bladder cancer, prostate cancer, Ewing sarcoma, glioblastoma and acute myeloid leukemia. Even more particularly, the present invention provides compounds capable of degrading CDK12 and/or Cyclin K for the treatment of lung cancer, breast cancer, liver cancer, colorectal cancer, gastric cancer, prostate cancer and leukemia.

Cyclin-dependent kinase (CDK) 12 (CDK12, gene id 51755) is a member of the subset of the CDK serine/threonine kinase family that phosphorylates the C-terminal domain (CTD) of RNA polymerase 11. CDK12 in complex with Cyclin K (CCNK, gene id 8812) regulates transcriptional, co- and posttranscriptional processes by phosphorylation of Ser2 and Ser5 of the CTD of RNA polymerase II complexes which are important in the elongation phase of pre-mRNA synthesis. CDK12/Cyclin K has been reported to regulate transcriptional elongation and mRNA processing, in particular co- and post-transcriptional pre-mRNA splicing, alternative splicing, 3′end processing, and suppression of intronic polyadenlyation. CDK13 (CDK13, gene id 8621), a kinase which is closely related to CDK12, also forms a complex with Cyclin K and regulates the transcription of a different set of genes (Bartkowiak et al. Genes Dev. 2010; 24:2303-16. Dubbury et al. Nature. 2018; 564:141-5. Greenleaf, Transcription. 2018; 10:91-110. Greifenberg et al. Cell Rep. 2016; 14:320-31. Liang et al. Mol. Cell. Biol. 2015; 35:928-38. Lui et al. J. Clin. Pathol. 2018; 71:957-62. Tien et al. Nuc. Acids Res. 2017; 45:6698-716). The transcription of genes encoding components of DNA damage signaling and repair pathways, such as the homologous recombination and replication stress response genes BRCA1, FANCD2, FANCI, and ATR, as well as encoding components of other stress response pathways, such as NF-κB and oxidative stress response, has been reported to be specifically regulated by CDK12/Cyclin K as demonstrated by gene knock-down and chemoproteomics studies (Blazek et al. Genes Dev. 2011; 25:2158-72. Henry et al. Sci. Signal. 2018; 11:eaam8216. Li et al. Sci. Rep. 2016; 6:21455.). In addition, CDK12/Cyclin K has been reported to control the translation of a subset of mRNAs, including the CHK1 mRNA, by directly phosphorylating the mRNA 5′ cap-binding translational repressor 4E-BP1 leading to its release from the mRNA cap (Choi et al. Genes Dev. 2019; 33:418-35). The recent discovery of rare bi-allelic CDK12 inactivating mutations in high-grade serous ovarian cancer and in primary and castration-resistant prostate cancer leading to a special type of genomic instability which is characterized by the occurance of numerous tandem duplications, indicating gross defects in DNA repair, underscores the role of CDK12 in DNA damage response and the maintenance of the genome (Ekumi et al. Nucl. Acids Res. 2015; 43:2575-89. Grasso et al. Nature. 2012; 487:239-43. Joshi et al. J. Biol. Chem. 2014; 289:9247-53. Menghi et al. Cancer Cell. 2018; 34:197-210.e5. Popova et al. Cancer Res. 2016; 76:1882-91. Quigley et al. Cell. 2018; 174:758-69.e9. Robinson et al. 2015; 162:454. Viswanathan et al. Cell. 2018; 174:433-47.e19. Wu et al. Cell. 2018; 173:1770-82.e14). The CDK12 gene is located on chromosome 17 about 200 kb proximal to the ERBB2 gene and is often coamplified in breast cancer. Furthermore, CDK12 gene amplification has been observed in other cancer types such as stomach cancer, esophageal cancer, pancreatic cancer, uterine cancer, endometrial cancer, prostate cancer, and bladder cancer (Lui et al. J Clin Pathol. 2018; 71:957-62. Gupta et al. Clin. Cancer Res. 2017; 23:1346-57). CDK12 amplification and high expression levels suggest a tumor promoting role of CDK12 which is, at least partially, based on alterantively spliced mRNAs, increased DNA repair capabilities and increased stress tolerance (Lui et al. J Clin Pathol. 2018; 71:957-62. Tien et al. Nucl. Acids Res. 2017; 45:6698-716). Taken together these data validated CDK12 as a potential target to develop drugs for the treatment of cancer and other diseases such as myotonic dystrophy type 1.

Some inhibitors of CDK12 kinase activity are known:

Flavopiridol, a micromolar non-selective inhibitor of CDK12 which inhibits other kinases such as CDK9, CDK1, CDK4 etc. (Bösken et al. Nat. Comm. 2014; 5:3505). Dinaciclib, a pan CDK inhibitor (Johnson et al. Cell Rep. 2016; 17:2367-81). THZ531, a dual inhibitor of CDK12 and CDK13 (Zhang et al. Nat. Chem. Biol. 2016; 12:876-84). SR-3029 and related purine compounds (Johannes et al. Chem. Med. Chem. 2018; 13:231-5). SR-4835, a dual inhibitor of CDK12 and CDK13 (Quereda et al. Cancer Cell 2019; 36:1-14). Compound 919278, a micromolar CDK12 inhibitor (Henry et al. Science Signal. 2018; 11:eaam8216). Arylurea derivatives (Ito et al. J. Med. Chem. 2018; 61:7710-28).

There is a need for development of compounds selectively impairing the function of CDK12/Cycdin K for the treatment of cancer and other diseases, e.g. by inducing the proteolytic degradation of CDK12 and/or Cyclin K protein in the cell. Restricted selectivity of inhibitors targeting the ATP pocket is an issue which may lead to undesired side effects and limited clinical utility (Sawa. Mini-Rev. Med. Chem 2008; 8:1291-7). Surprisingly, the compounds described in the present invention induce the proteolytic degradation of CDK12 and/or Cyclin K protein in the cell. CDK12 inhibitors with low kinase inhibition potential at physiological ATP concentrations but strong CDK12 degrading potency are selective against other kinases. In addition, by degradation of CDK12 and/or Cyclin K functions of the CDK12/CyclinK protein complex which are independent from the sole kinase activity, such as scaffolding functions for other proteins e.g. in the RNA polymerase II complex or the pre-mRNA splicing complex will be impaired as well. Thus, there is a need to provide compounds which impair the activity of CDK12/Cyclin K in the cell and which exhibit a good degree of selectivity towards the targeting of other CDKs and other kinases, such as, for example, casein kinases.

### SUMMARY

The present invention provides compounds of general formula (I):

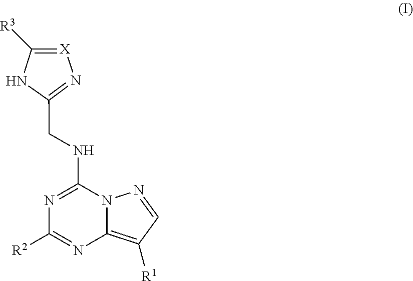

in which X, R¹, R² and R³ are as described and defined herein, methods of preparing said compounds, intermediate compounds useful for preparing said compounds, pharmaceutical compositions and combinations comprising said compounds, and the use of said compounds for manufacturing pharmaceutical compositions for the treatment and/or prophylaxis of diseases, in particular of hyperproliferative disorders such as cancer disorders, as a sole agent or in combination with other active ingredients.

### DESCRIPTION OF THE INVENTION

It has now been found that the compounds of the present invention effectively impair the activity of CDK12/Cyclin K for which data are given in the biological experimental section and may therefore be used for the treatment and/or prophylaxis of hyperproliferative disorders, such as cancer disorders. In particular, the compounds of the present invention are CDK12 inhibitors with low kinase inhibition potential at physiological ATP concentrations but strong proteolytic CDK12 and/or Cyclin K degrading potency in cells and are therefore selective against other kinases while maintaining an impairing effect towards CDK12/Cyclin K.

In accordance with a first aspect, the present invention provides compounds of general formula (I):

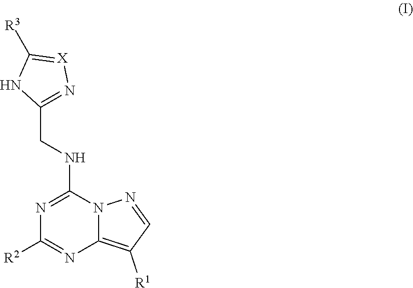

wherein
R¹ is selected from a halogen atom, a C₁-C₆-alkyl group, a C₁-C₆-haloalkyl group, a C₃-C₆-cycloalkyl group, a (C₃-C₆-cycloalkyl)-(C₁-C₆-alkyl)- group, a cyano group, a phenyl group, a heterocycloalkyl group and a heteroaryl group,
   wherein said C₁-C₆-alkyl, C₃-C₆-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₆-cycloalkyl group, a C₃-C₆-cycloalkoxy group and a R⁵R⁶N— group;
R² is selected from a C₁-C₆-alkyl group, a C₁-C₆-alkoxy group, a C₁-C₆-haloalkyl group, a C₁-C₆-haloalkoxy group, a C₃-C₆-cycloalkyl group, a C₃-C₈-cycloalkoxy group, a (C₃-C₆-cycloalkyl)-(C₁-C₂-alkyl)-O— group, a (C₃-C₆-hydroxyalkyl)-(C₁-C₂-alkyl)-O— group, a (C₃-C₆-alkoxyalkyl)-(C₁-C₂-alkyl)-O— group, a ((CH₃)₂N)—(C₁-C₂-alkyl)-O— group, a heterocycloalkyl group, a (heterocycloalkyl)-O— group and a —NR^{a}R^{b} group,
   wherein said heterocycloalkyl group is connected to the rest of the molecule via a carbon atom of said heterocycloalkyl group,
   wherein R^{a} and R^{b} are each independently selected from a hydrogen atom, a C₁-C₆-alkyl group, a C₃-C₈-cycloalkyl group, a C₁-C₆-haloalkyl group, a (C₃-C₆-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
      wherein said C₃-C₈-cycloalkyl, phenyl, heteroaryl or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group and a R⁵R⁶N— group,
      wherein at least one of R^{e} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from cyano, hydroxy, a C₁-C₆ alkyl group, a C₁-hydroxyalkyl group, a C₁-C₂haloalkyl group, a C₁-C₂-alkoxy group, a C₃-C₄-cycloalkyl group, a (C₁-C₂alkyl)OOC— group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁—C— alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R^{a} is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

### DETAILED DESCRIPTION

### Definitions

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom. Commonly, it is possible for the number of optional substituents, when present, to be 1, 2, 3, 4 or 5, in particular 1, 2 or 3, more particularly 1 or 2, and even more particularly 1.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

When groups in the compounds according to the invention are substituted, it is possible for said groups to be mono-substituted or poly-substituted with substituent(s), unless otherwise specified. Within the scope of the present invention, the meanings of all groups which occur repeatedly are independent from one another. It is possible that groups in the compounds according to the invention are substituted with one, two or three identical or different substituents, particularly with one, two or three substituents, more particularly with one substituent.

The terms “oxo”, “an oxo group” or “an oxo substituent” mean a doubly bonded oxygen atom ═O. Oxo may be attached to atoms of suitable valency, for example to a saturated carbon atom or to a sulfur atom. For example, but without limitation, one oxo group can be attached to a carbon atom, resulting in the formation of a carbonyl group C(═O), or two oxo groups can be attached to one sulfur atom, resulting in the formation of a sulfonyl group —S(═O)₂.

The term “ring substituent” means a substituent attached to an aromatic or nonaromatic ring which replaces an available hydrogen atom on the ring.

Should a composite substituent be composed of more than one parts, e.g. (C₁-C₄-alkoxy)-(C₁-C₄-alkyl)-, it is possible for the position of a given part to be at any suitable position of said composite substituent, i.e. the C₁-C₄-alkoxy part can be attached to any carbon atom of the C₁-C₄-alkyl part of said (C₁-C₄-alkoxy)-(C₁-C₄-alkyl)- group. A hyphen at the beginning or at the end of such a composite substituent indicates the point of attachment of said composite substituent to the rest of the molecule. Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

If within the present text any item is referred to as “supra” within the description it indicates any of the respective disclosures made within the specification in any of the preceding pages, or above on the same page.

If within the present text any item is referred to as “infra” within the description it indicates any of the respective disclosures made within the specification in any of the subsequent pages, or below on the same page.

The terms as mentioned in the present text have the following meanings:

The term “halogen atom” means a fluorine, chlorine, bromine or iodine atom, particularly a fluorine, chlorine or bromine atom, more particularly a fluorine atom.

The term “C₁-C₆-alkyl” means a linear or branched, saturated, monovalent hydrocarbon group having 1, 2, 3, 4, 5 or 6 carbon atoms, e.g. a methyl-, ethyl-, propyl-, isopropyl-, butyl-, sec-butyl-, isobutyl-, tert-butyl-, pentyl-, isopentyl-, 2-methylbutyl-, 1-methylbutyl-, 1-ethylpropyl-, 1,2-dimethylpropyl-, neo-pentyl-, 1,1-dimethylpropyl-, hexyl-, 1-methylpentyl-, 2-methylpentyl-, 3-methylpentyl-, 4-methylpentyl-, 1-ethylbutyl-, 2-ethylbutyl-, 1,1-dimethylbutyl-, 2,2-dimethylbutyl-, 3,3-dimethylbutyl-, 2,3-dimethylbutyl-, 1,2-dimethylbutyl- or a 1,3-dimethylbutyl- group, or an isomer thereof. Particularly, said group has 1, 2, 3 or 4 carbon atoms (“C₁-C₄-alkyl”), e.g. a methyl-, ethyl-, propyl-, isopropyl-, butyl-, sec-butyl-, isobutyl- or a tert-butyl group, more particularly 1, 2 or 3 carbon atoms (“C₁-C₃-alkyl”), e.g. a methyl-, ethyl-, n-propyl- or an isopropyl group.

The term “C₁-C₆-hydroxyalkyl” means a linear or branched, saturated, monovalent hydrocarbon group in which the term “C₁-C₆-alkyl” is defined supra, and in which one or more hydrogen atoms are replaced with a hydroxy group, e.g. a hydroxymethyl-, 1-hydroxyethyl-, 2-hydroxyethyl-, 1,2-dihydroxyethyl-, 3-hydroxypropyl-, 2-hydroxypropyl-, 1-hydroxypropyl-, 1-hydroxypropan-2-yl-, 2-hydroxypropan-2-yl-, 2,3-dihydroxypropyl-, 1,3-dihydroxypropan-2-yl-, 3-hydroxy-2-methyl-propyl-, 2-hydroxy-2-methyl-propyl- or a 1-hydroxy-2-methyl-propyl- group.

The term “C₁-C₆-alkylsulfanyl” means a linear or branched, saturated, monovalent group of formula (C₁-C₆-alkyl)-S—, in which the term “C₁-C₆-alkyl” is as defined supra, e.g. a methylsulfanyl-, ethylsulfanyl-, propylsulfanyl-, isopropylsulfanyl-, butylsulfanyl-, sec-butylsulfanyl-, isobutylsulfanyl-, tert-butylsulfanyl-, pentylsulfanyl-, isopentylsulfanyl- or a hexylsulfanyl- group.

The term “C₁-C₆-haloalkyl” means a linear or branched, saturated, monovalent hydrocarbon group in which the term “C₁-C₆-alkyl” is as defined supra and in which one or more of the hydrogen atoms are replaced, identically or differently, with a halogen atom. Preferably, said halogen atom is a fluorine atom. Said C₁-C₆-haloalkyl, particularly a C₁-C₃-haloalkyl group is, for example, fluoromethyl-, difluoromethyl-, trifluoromethyl-, 2-fluoroethyl-, 2,2-difluoroethyl-, 2,2,2-trifluoroethyl-, pentafluoroethyl-, 3,3,3-trifluoropropyl- or a 1,3-difluoropropan-2-yl group.

The term “C₁-C₆-alkoxy” means a linear or branched, saturated, monovalent group of formula (C₁-C₆-alkyl)-O—, in which the term “C₁-C₆-alkyl” group is as defined supra, e.g. methoxy-, ethoxy-, n-propoxy-, isopropoxy-, n-butoxy-, sec-butoxy-, isobutoxy-, tert-butoxy-, pentyloxy-, isopentyloxy- or a n-hexyloxy group, or an isomer thereof.

The term “C₁-C₆-haloalkoxy” means a linear or branched, saturated, monovalent C₁-C₆-alkoxy group, as defined supra, in which one or more of the hydrogen atoms is replaced, identically or differently, with a halogen atom. Preferably, said halogen atom in “C₁-C₆-haloalkoxy-” is fluorine, resulting in a group referred herein as “C₁-C₆-fluoroalkoxy-”. Representative C₁-C₆-fluoroalkoxy- groups include, for example, —OCF₃, —OCHF₂, —OCH₂F, —OCF₂CF₃ and —OCH₂CF₃.

The term “C₂-C₆-alkenyl-” means a linear or branched, monovalent hydrocarbon group, which contains one or more double bonds and which has 2, 3, 4, 5 or 6 carbon atoms, preferably 2, 3 or 4 carbon atoms (“C₂-C₄-alkenyl-”) or 2 or 3 carbon atoms (“C₂-C₃-alkenyl-”), it being understood that in the case in which said alkenyl- group contains more than one double bond, then said double bonds may be isolated from, or conjugated with, each other. Representative alkenyl groups include, for example, an ethenyl-, prop-2-enyl-, (E)-prop-1-enyl-, (Z)-prop-1-enyl-, iso-propenyl-, but-3-enyl-, (E)-but-2-enyl-, (Z)-but-2-enyl-, (E)-but-1-enyl-, (Z)-but-1-enyl-, 2-methylprop-2-enyl-, 1-methylprop-2-enyl-, 2-methylprop-1-enyl-, (E)-1-methylprop-1-enyl-, (Z)-1-methylprop-1-enyl-, buta-1,3-dienyl-, pent-4-enyl-, (E)-pent-3-enyl-, (Z)-pent-3-enyl-, (E)-pent-2-enyl-, (Z)-pent-2-enyl-, (E)-pent-1-enyl-, (Z)-pent-1-enyl-, 3-methylbut-3-enyl-, 2-methylbut-3-enyl-, 1-methylbut-3-enyl-, 3-methylbut-2-enyl-, (E)-2-methylbut-2-enyl-, (Z)-2-methylbut-2-enyl-, (E)-1-methylbut-2-enyl-, (Z)-1-methylbut-2-enyl-, (E)-3-methylbut-1-enyl-, (Z)-3-methylbut-1-enyl-, (E)-2-methylbut-1-enyl-, (Z)-2-methylbut-1-enyl-, (E)-1-methylbut-1-enyl-, (Z)-1-methylbut-1-enyl-, 1,1-dimethylprop-2-enyl-, 1-ethylprop-1-enyl-, 1-propylvinyl-, 1-isopropylvinyl-, (E)-3,3-dimethylprop-1-enyl-, (Z)-3,3-dimethylprop-1-enyl-, penta-1,4-dienyl-, hex-5-enyl-, (E)-hex-4-enyl-, (Z)-hex-4-enyl-, (E)-hex-3-enyl-, (Z)-hex-3-enyl-, (E)-hex-2-enyl-, (Z)-hex-2-enyl-, (E)-hex-1-enyl-, (Z)-hex-1-enyl-, 4-methylpent-4-enyl-, 3-methylpent-4-enyl-, 2-methylpent-4-enyl-, 1-methylpent-4-enyl-, 4-methylpent-3-enyl-, (E)-3-methylpent-3-enyl-, (Z)-3-methylpent-3-enyl-, (E)-2-methylpent-3-enyl-, (Z)-2-methylpent-3-enyl-, (E)-1-methylpent-3-enyl-, (Z)-1-methylpent-3-enyl-, (E)-4-methylpent-2-enyl-, (Z)-4-methylpent-2-enyl-, (E)-3-methylpent-2-enyl-, (Z)-3-methylpent-2-enyl-, (E)-2-methylpent-2-enyl-, (Z)-2-methylpent-2-enyl-, (E)-1-methylpent-2-enyl-, (Z)-1-methylpent-2-enyl-, (E)-4-methylpent-1-enyl-, (Z)-4-methylpent-1-enyl-, (E)-3-methylpent-1-enyl-, (Z)-3-methylpent-1-enyl-, (E)-2-methylpent-1-enyl-, (Z)-2-methylpent-1-enyl-, (E)-1-methylpent-1-enyl-, (Z)-1-methylpent-1-enyl-, 3-ethylbut-3-enyl-, 2-ethylbut-3-enyl-, 1-ethylbut-3-enyl-, (E)-3-ethylbut-2-enyl-, (Z)-3-ethylbut-2-enyl-, (E)-2-ethylbut-2-enyl-, (Z)-2-ethylbut-2-enyl-, (E)-1-ethylbut-2-enyl-, (Z)-1-ethylbut-2-enyl-, (E)-3-ethylbut-1-enyl-, (Z)-3-ethylbut-1-enyl-, 2-ethylbut-1-enyl-, (E)-1-ethylbut-1-enyl-, (Z)-1-ethylbut-1-enyl-, 2-propylprop-2-enyl-, 1-propylprop-2-enyl-, 2-isopropylprop-2-enyl-, 1-isopropylprop-2-enyl-, (E)-2-propylprop-1-enyl-, (Z)-2-propylprop-1-enyl-, (E)-1-propylprop-1-enyl-, (Z)-1-propylprop-1-enyl-, (E)-2-isopropylprop-1-enyl-, (Z)-2-isopropylprop-1-enyl-, (E)-1-isopropylprop-1-enyl-, (Z)-1-isopropylprop-1-enyl-, hexa-1,5-dienyl- and a 1-(1,1-dimethylethyl-)ethenyl group. Particularly, said group is an ethenyl- or a prop-2-enyl group.

The same definitions can be applied should the alkenyl group be placed within a chain as a bivalent “C₂-C₆-alkenylene” moiety. All names as mentioned above then will bear a “ene” added to their end, thus e.g., a “pentenyl” becomes a bivalent “pentenylene” group.

The term “C₂-C₆-haloalkenyl-” means a linear or branched hydrocarbon group in which one or more of the hydrogen atoms of a “C₂-C₆-alkenyl-” as defined supra are each replaced, identically or differently, by a halogen atom. Preferably, said halogen atom is fluorine, resulting in a group referred herein as “C₂-C₆-fluoroalkenyl-”. Representative C₂-C₆-fluoroalkenyl- groups include, for example, —CH═CF₂, —CF═CH₂, —CF═CF₂, —C(CH₃)═CF₂, —CH═C(F)—CH₃, —CH₂—CF═CF₂ and —CF₂—CH═CH₂.

The term “C₂-C₆-alkynyl-” means a linear or branched, monovalent hydrocarbon group which contains one or more triple bonds, and which contains 2, 3, 4, 5 or 6 carbon atoms, preferably 2, 3 or 4 carbon atoms (“C₂-C₄-alkynyl-”) or 2 or 3 carbon atoms (“C₂-C₃-alkynyl-”). Representative C₂-C₆-alkynyl- groups include, for example, an ethynyl-, prop-1-ynyl-, prop-2-ynyl-, but-1-ynyl-, but-2-ynyl-, but-3-ynyl-, pent-1-ynyl-, pent-2-ynyl, pent-3-ynyl-, pent-4-ynyl-, hex-1-ynyl-, hex-2-ynyl-, hex-3-ynyl-, hex-4-ynyl-, hex-5-ynyl-, 1-methylprop-2-ynyl-, 2-methylbut-3-ynyl-, 1-methylbut-3-ynyl-, 1-methylbut-2-ynyl-, 3-methylbut-1-ynyl-, 1-ethylprop-2-ynyl-, 3-methylpent-4-ynyl-, 2-methylpent-4-ynyl-, 1-methylpent-4-ynyl-, 2-methylpent-3-ynyl-, 1-methylpent-3-ynyl-, 4-methylpent-2-ynyl-, 1-methylpent-2-ynyl-, 4-methylpent-1-ynyl-, 3-methylpent-1-ynyl-, 2-ethylbut-3-ynyl-, 1-ethylbut-3-ynyl-, 1-ethylbut-2-ynyl-, 1-propylprop-2-ynyl-, 1-isopropylprop-2-ynyl-, 2,2-dimethylbut-3-ynyl-, 1,1-dimethylbut-3-ynyl-, 1,1-dimethylbut-2-ynyl- and a 3,3-dimethylbut-1-ynyl- group. Particularly, said alkynyl- group is an ethynyl-, a prop-1-ynyl- or a prop-2-ynyl group.

The term “C₃-C₈-cycloalkyl” means a saturated, monovalent, mono- or bicyclic hydrocarbon ring which contains 3, 4, 5, 6, 7 or 8 carbon atoms (“C₃-C₈-cycloalkyl”). Analogously, the term “C₃-C₆-cycloalkyl” means a saturated, monovalent, mono- or bicyclic hydrocarbon ring which contains 3, 4, 5 or 6 carbon atoms (“C₃-C₆-cycloalkyl”). Said C₃-C₈-cycloalkyl or C₃-C₆-cycloalkyl group is for example, a monocyclic hydrocarbon ring, e.g. a cyclopropyl-, cyclobutyl-, cyclopentyl-, cyclohexyl-, cycloheptyl- or cyclooctyl- group, or a bicyclic hydrocarbon ring, e.g. a bicyclo[4.2.0]octyl- or a octahydropentalenyl- group.

The term “C₃-C₆-halocycloalkyl” means a saturated, monovalent hydrocarbon ring which contains 3, 4, 5 or 6 carbon atoms in which the term “C₃-C₆-cycloalkyl” is as defined supra and in which one or more of the hydrogen atoms of the hydrocarbon ring are replaced, identically or differently, with a halogen atom. Preferably, said halogen atom is a fluorine atom. The “C₃-C₆-cycloalkyl” group as defined supra in which one or more of the hydrogen atoms are replaced, identically or differently, with a halogen atom, preferably a fluorine atom, is for example and preferably, a monocyclic hydrocarbon ring, e.g. a cyclopropyl-, cyclobutyl-, cyclopentyl-, cyclohexyl- group.

The term “C₄-C₃-cycloalkenyl” means a monovalent, mono- or bicyclic hydrocarbon ring which contains 4, 5, 6, 7 or 8 carbon atoms and one double bond. Particularly, said ring contains 4, 5 or 6 carbon atoms (“C₄-C₆-cycloalkenyl”). Said C₄-C₃-cycloalkenyl group is for example, a monocyclic hydrocarbon ring, e.g., a cyclobutenyl-, cyclopentenyl-, cyclohexenyl-, cycloheptenyl- or a cyclooctenyl group, or a bicyclic hydrocarbon ring, e.g., a bicyclo[2.2.1]hept-2-enyl- or a bicyclo[2.2.2]oct-2-enyl group.

The term “C₃-C₈-cycloalkoxy” means a saturated, monovalent, mono- or bicyclic group of formula (C₃-C₈-cycloalkyl)-O—, which contains 3, 4, 5, 6, 7 or 8 carbon atoms, in which the term “C₃-C₈-cycloalkyl” is defined supra, e.g. a cyclopropyloxy-, cyclobutyloxy-, cyclopentyloxy-, cyclohexyloxy-, cycloheptyloxy- or a cyclooctyloxy- group.

If the term “heterocycloalkyl” is used without specifying a number of atoms it is meant to be a “4- to 10-membered heterocycloalkyl-” group, more particularly a 5- to 6-membered heterocycloalkyl group. The terms “4- to 7-membered heterocycloalkyl”, “4- to 6-membered heterocycloalkyl” and “5- to 7-membered heterocycloalkyl” mean a monocyclic, saturated heterocycle with “4, 5, 6 or 7” or, respectively, “4, 5 or 6” or “5, 6 or 7” ring atoms in total, which are saturated or partially unsaturated monocycles, bicycles or polycycles that contain one or two identical or different ring heteroatoms selected from nitrogen, oxygen and sulfur or one group selected from —S(═O)—, —S(═O)₂— and —S(═O)(═NH)—. It is possible for said heterocycloalkyl group to be attached to the rest of the molecule via any one of the carbon atoms or, if present, a nitrogen atom.

Exemplarily, without being limited thereto, said “4- to 7-membered heterocycloalkyl”, can be a 4-membered ring, a “4-membered heterocycloalkyl-” group, such as an azetidinyl- or an oxetanyl group; or a 5-membered ring, a “5-membered heterocycloalkyl-” group, such as a tetrahydrofuranyl-, dioxolinyl-, pyrrolidinyl-, imidazolidinyl-, pyrazolidinyl- or a pyrrolinyl group; or a 6-membered ring, a “6-membered heterocycloalkyl-” group, such as a tetrahydropyranyl-, piperidinyl-, morpholinyl-, 3-oxomorpholin-4-yl, dithianyl-, thiomorpholinyl- or a piperazinyl group; or a 7-membered ring, a “7-membered heterocycloalkyl-” group, such as an azepanyl-, diazepanyl- or an oxazepanyl group, for example. The heterocycloalkyl groups may be substituted one or more times independently with C₁-C₃-alkyl, C₁-C₃-alkoxy, hydroxy, halogen or a carbonyl group.

Particularly, “4- to 6-membered heterocycloalkyl” means a 4- to 6-membered heterocycloalkyl as defined supra containing one ring nitrogen atom and optionally one further ring heteroatom selected from nitrogen, oxygen and sulfur. Particularly, “5- to 7-membered heterocycloalkyl” means a 5- to 7-membered heterocycloalkyl as defined supra containing one ring nitrogen atom and optionally one further ring heteroatom selected from nitrogen, oxygen and sulfur. More particularly, “5- or 6-membered heterocycloalkyl” means a monocyclic, saturated heterocycle with 5 or 6 ring atoms in total, containing one ring nitrogen atom and optionally one further ring heteroatom selected from nitrogen and oxygen.

The term “heteroaryl-” means a monocyclic, bicyclic or tricyclic aromatic ring system having 5, 6, 7, 8, 9, 10, 11, 12, 13 or 14 ring atoms (a “5- to 14-membered heteroaryl-” group), preferably 5, 6, 9 or 10 ring atoms and which contains 1, 2, 3 or 4 heteroatoms which may be identical or different, said heteroatoms being selected from oxygen, nitrogen and sulfur. Said heteroaryl- group can be a 5-membered heteroaryl group, such as, for example, a thienyl-, furanyl-, pyrrolyl-, oxazolyl-, thiazolyl-, imidazolyl-, pyrazolyl-, isoxazolyl-, isothiazolyl-, oxadiazolyl-, triazolyl-, thiadiazolyl- or a tetrazolyl group; or a 6-membered heteroaryl group, such as, for example, a pyridyl-, pyridazinyl-, pyrimidyl-, pyrazinyl- or a triazinyl group; or a benzo-fused 5-membered heteroaryl- group, such as, for example, a benzofuranyl-, benzothienyl-, benzoxazolyl-, benzisoxazolyl-, benzimidazolyl-, benzothiazolyl-, benzotriazolyl-, indazolyl-, indolyl- or a isoindolyl group; or a benzo-fused 6-membered heteroaryl group, such as, for example, a quinolinyl-, quinazolinyl-, isoquinolinyl-, cinnolinyl-, phthalazinyl- or quinoxalinyl-; or another bicyclic group, such as, for example, indolizinyl-, purinyl- or a pteridinyl group.

Preferably, “heteroaryl-” is a monocyclic aromatic ring system having 5 or 6 ring atoms and which contains at least one heteroatom, if more than one, they may be identical or different, said heteroatom being selected from oxygen, nitrogen and sulfur, a (“5- to 6-membered monocyclic heteroaryl-”) group, such as, for example, a thienyl-, furanyl-, pyrrolyl-, oxazolyl-, thiazolyl-, imidazolyl-, pyrazolyl-, isoxazolyl-, isothiazolyl-, oxadiazolyl-, triazolyl-, thiadiazolyl-, tetrazolyl-, pyridyl-, pyridazinyl-, pyrimidyl-, pyrazinyl- or a triazinyl group.

In particular, in the context of the present invention, when applied to any of the substituents of the compounds of general formula (I), the term “heteroaryl” is to be understood as meaning preferably a monocyclic aromatic ring system having 5 or 6 ring atoms and which contains one, two or three heteroatoms, preferably one or two heteroatoms, which may be identical or different, said heteroatom(s) being independently selected from oxygen, sulphur and nitrogen, preferably from oxygen and nitrogen, i.e. a (“5- to 6-membered monocyclic heteroaryl-”) group.

In general, and unless otherwise mentioned, said heteroaryl- groups include all the possible isomeric forms thereof, e.g., the positional isomers thereof. Thus, for some illustrative non-restricting example, the term pyridyl-includes pyridin-2-yl-, pyridin-3-yl- and pyridin-4-yl-; the term thienyl-includes thien-2-yl- and thien-3-yl-, and a heteroarylene group may be inserted into a chain also in the inverse way such as e.g. a 2,3-pyridinylene includes pyridine-2,3-yl as well as pyridine-3,2-yl. Furthermore, said heteroaryl- groups can be attached to the rest of the molecule via any one of the carbon atoms, or, if applicable, a nitrogen atom, e.g., a pyrrol-1-yl-, a pyrazol-1-yl- or an imidazol-1-yl- group.

Particularly, the heteroaryl group is a pyridyl- or pyrimidyl group or a imidazolyl group, including a hydroxy substitution of the pyridyl group leading, e.g., to a 2-hydroxy-pyridine which is the tautomeric form to a 2-oxo-2(1H)-pyridine. In some embodiments, the heteroaryl group is an oxazolyl group.

Further, as used herein, the term “C₃-C₈”, as used throughout this text, e.g., in the context of the definition of “C₃-C₈-cycloalkyl-”, is to be understood as meaning e.g. a cycloalkyl- group having a whole number of carbon atoms of 3 to 8, i.e., 3, 4, 5, 6, 7 or 8 carbon atoms. It is to be understood further that said term “C₃-C₈” is to be interpreted as disclosing any sub-range comprised therein, e.g., C₃-C₆, C₄-C₅, C₃-C₆, C₃-C₄, C₄-C₆, C₅-C₇, preferably C₃-C₆.

Similarly, as used herein, the term “C₂-C₆”, as used throughout this text, e.g., in the context of the definitions of “C₂-C₆-alkenyl-” and “C₂-C₆-alkynyl-”, is to be understood as meaning an alkenyl- group or an alkynyl- group having a whole number of carbon atoms from 2 to 6, i.e., 2, 3, 4, 5 or 6 carbon atoms. It is to be understood further that said term “C₂-C₆” is to be interpreted as disclosing any sub-range comprised therein, e.g., C₂-C₆, C₃-C₆, C₃-C₄, C₂-C₃, C₂-C₄, C₂-C₅ preferably C₂-C₃.

The term “C₁-C₆”, as used throughout this text, e.g., in the context of the definition of “C₁-C₆-alkyl-”, “C₁-C₆-haloalkyl-”, “C₁-C₆-alkoxy-” or “C₁-C₆-haloalkoxy-” is to be understood as meaning an alkyl group having a whole number of carbon atoms from 1 to 6, i.e., 1, 2, 3, 4, 5 or 6 carbon atoms. It is to be understood further that said term “C₁-C₆” is to be interpreted as disclosing any sub-range comprised therein, e.g. C₁-C₆, C₂-C₅, C₃-C₄, C₁-C₂, C₁-C₃, C₁-C₄, C₁-C₅, C₁-C₆ preferably C₁-C₂, C₁-C₃, C₁-C₄, C₁-C₅, C₁-C₆ more preferably C₁-C₄, in the case of “C₁-C₆-haloalkyl-” or “C₁-C₆-haloalkoxy-” even more preferably C₁-C₂.

When a range of values is given, said range encompasses each value and sub-range within said range.

For example:

“C₁-C₆” encompasses C₁, C₂, C₃, C₄, C₅, C₆, C₁-C₆, C₁-C₆, C₁-C₄, C₁-C₃, C₁-C₂, C₂- C₆, C₂-C₅, C₂-C₄, C₂-C₃, C₃-C₆, C₃-C₆, C₃-C₄, C₄-C₆, C₄-C₅, and C₅-C₆;
“C₂-C₆” encompasses C₂, C₃, C₄, C₅, C₆, C₂-C₆, C₂-C₅, C₂-C₄, C₂-C₃, C₃-C₆, C₃-C₆, C₃-C₄, C₄-C₆, C₄-C₅, and C₅-C₆;
“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₈, C₃-C₈, C₃-C₇, C₃-C₆, C₃-C₈, C₃-C₄, C₄- C₈, C₄-C₇, C₄-C₆, C₄-C₅, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₆, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₅, C₅-C₇, C₅-C₆ and C₆-C₇;
“C₄-C₆” encompasses C₄, C₅, C₆, C₄-C₆, C₄-C₅ and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

In particular embodiments for the compounds of formula (I) of the present invention, reference is made to the fact that, when substituted, a particular phenyl group is preferably substituted in one or more of the ortho- and/or meta-positions with respect to the point of attachment of said phenyl group to the rest of the molecule. This is shown below, where the ortho- and meta-positions in which the phenyl group is preferably substituted with respect to the point of attachment to the rest of the molecule are marked (*):

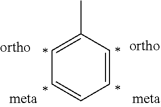

In particular, preferred embodiments of the compounds of formula (I) of the present invention may comprise a phenyl group as substituent R³ which is, when substituted, preferably substituted in one or more of the ortho- and/or meta-positions (marked with * below) with respect to the point of attachment of said phenyl group to the rest of the molecule:

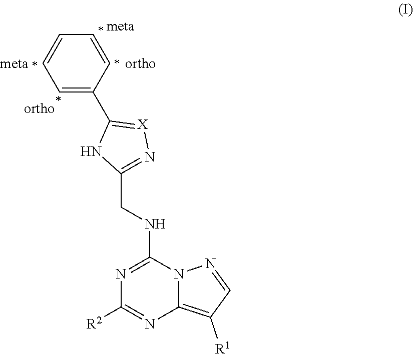

As used herein, the term “leaving group” refers to an atom or a group of atoms that is displaced in a chemical reaction as stable species taking with it the bonding electrons, e.g., typically forming an anion. Preferably, a leaving group is selected from the group comprising: halo, in particular a chloro, bromo or iodo, (methylsulfonyl)oxy-, [(4-methylphenyl)sulfonyl]oxy-, [(trifluoromethyl)sulfonyl]oxy-, [(nonafluorobutyl)sulfonyl]oxy-[(4-bromophenyl)sulfonyl]oxy-, [(4-nitrophenyl)sulfonyl]oxy-, [(2-nitro-phenyl)sulfonyl]oxy-, [(4-isopropylphenyl)sulfonyl]oxy-, [(2,4,6-triisopropylphenyl)sulfonyl]oxy-, [(2,4,6-trimethylphenyl)sulfonyl]oxy-, [(4-tert-butylphenyl)sulfonyl]oxy-, (phenylsulfonyl)oxy-, and a [(4-methoxyphenyl)sulfonyl]oxy group.

As used herein, the term “protective group” is a protective group attached to an oxygen or nitrogen atom in intermediates used for the preparation of compounds of the general formula (I). Such groups are introduced e.g., by chemical modification of the respective hydroxy or amino group in order to obtain chemoselectivity in a subsequent chemical reaction. Protective groups for hydroxy and amino groups are described for example in T. W. Greene and P. G. M. Wuts in Protective Groups in Organic Synthesis, 4^{th} edition, Wiley 2006; more specifically, protective groups for amino groups can be selected from substituted sulfonyl groups, such as a mesyl-, tosyl- or a phenylsulfonyl group, acyl groups such as a benzoyl-, acetyl- or a tetrahydropyranoyl group, or carbamate based groups, such as a tert-butoxycarbonyl group (Boc). Protective groups for hydroxy groups can be selected from acyl groups such as a benzoyl-, acetyl-, pivaloyl- or a tetrahydropyranoyl group, or can include silicon, as in e.g., a tert-butyldimethylsilyl-, tert-butyldiphenylsilyl-, triethylsilyl- or a triisopropylsilyl group.

The term “substituent” refers to a group “substituted” on, e.g., an alkyl-, haloalkyl-, cycloalkyl-, heterocyclyl-, heterocycloalkenyl-, cycloalkenyl-, aryl-, or a heteroaryl group at any atom of that group, replacing one or more hydrogen atoms therein. In one aspect, the substituent(s) on a group are independently any one single, or any combination of two or more of the permissible atoms or groups of atoms delineated for that substituent. In another aspect, a substituent may itself be substituted with any one of the above substituents. Further, as used herein, the phrase “optionally substituted” means unsubstituted (e.g., substituted with an H) or substituted.

It will be understood that the description of compounds herein is limited by principles of chemical bonding known to those skilled in the art. Accordingly, where a group may be substituted by one or more of a number of substituents, such substitutions are selected so as to comply with principles of chemical bonding with regard to valencies, etc., and to give compounds which are not inherently unstable. For example, any carbon atom will be bonded to two, three, or four other atoms, consistent with the four valence electrons of carbon.

By “subject” is meant a mammal, including, but not limited to, a human or non-human mammal, such as a bovine, equine, canine, ovine, rodent, or feline.

It is possible for the compounds of general formula (I) to exist as isotopic variants. The invention therefore includes one or more isotopic variant(s) of the compounds of general formula (I), particularly deuterium-containing compounds of general formula (I).

The invention also includes all suitable isotopic variations of a compound of the invention.

The term “isotopic variant” of a compound or a reagent is defined as a compound exhibiting an unnatural proportion of one or more of the isotopes that constitute such a compound.

The expression “unnatural proportion” in relation to an isotope means a proportion of such isotope which is higher than its natural abundance. The natural abundances of isotopes to be applied in this context are described in “Isotopic Compositions of the Elements 1997”, Pure Appl. Chem., 70(1), 217-235, 1998.

An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Accordingly, recitation of “hydrogen” or “H” should be understood to encompass ¹H (protium), ²H (deuterium), and ³H (tritium) unless otherwise specified. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

With respect to the treatment and/or prophylaxis of the disorders specified herein, the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful, e.g., in drug and/or substrate tissue distribution studies. These isotopes are particularly preferred for the ease of their incorporation and detectability. Positron-emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g., Nevirapine: A. M. Sharma et al., Chem. Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g., Rofecoxib: F. Schneider et al., Arzneim. Forsch./Drug. Res., 2006, 56, 295; Telaprevir: F. Maltais et al., J. Med. Chem., 2009, 52, 7993). Deuterated drugs showing this effect may have reduced dosing requirements (e.g., lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

Preferred isomers are those which produce the more desirable biological activity. These separated, pure or partially purified isomers or racemic mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

Further, it may be possible for the compounds of the present invention to exist as tautomers. For example, any compound of the present invention which contains an imidazopyridine moiety as a heteroaryl group for example can exist as a 1H tautomer, or a 3H tautomer, or even a mixture in any amount of the two tautomers, namely:

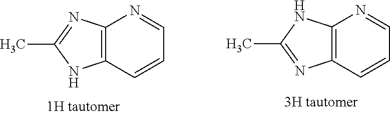

The present invention includes all possible tautomers of the compounds of the present invention as single tautomers, or as any mixture of said tautomers, in any ratio.

Further, in the context of the present invention, it may be possible for the compounds of formula (I) to exist as tautomers. For example, as depicted below, the compounds of formula (I) according to the present invention can exist as a 1H tautomer, or a 3H tautomer, or even a mixture in any amount of two or more of the possible tautomers:

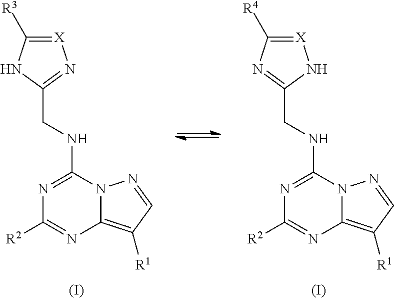

The present invention includes all possible tautomers of the compounds of formula (I) of the present invention as single tautomers, or as any mixture of any two or more of any possible tautomers, in any ratio.

Further, in the context of the present invention, it may be possible for the compounds of formula (I) where X is a nitrogen atom to exist as tautomers. For example, as depicted below, the compounds of formula (I) according to the present invention where X is a nitrogen atom can exist as a 1H tautomer, or a 4H tautomer, or even a mixture in any amount of two or more of the possible tautomers:

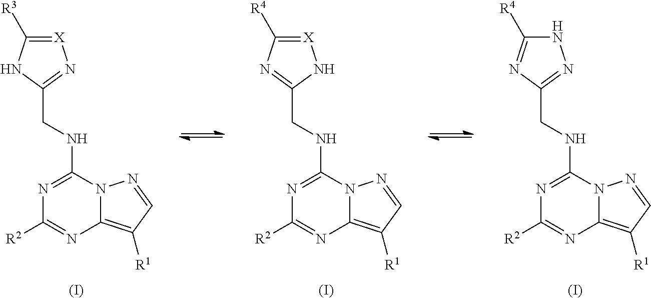

The present invention includes all possible tautomers of the compounds of formula (I) of the present invention where X is a nitrogen atom as single tautomers, or as any mixture of any two or more possible tautomers, in any ratio.

Further, in the context of the present invention, it may be possible for the compounds of formula (I) where X is a CR⁴ group to exist as tautomers. For example, as depicted below, the compounds of formula (I) according to the present invention where X is a CR₄ group can exist as two different 1H tautomers, or even a mixture in any amount of two or more of the possible tautomers:

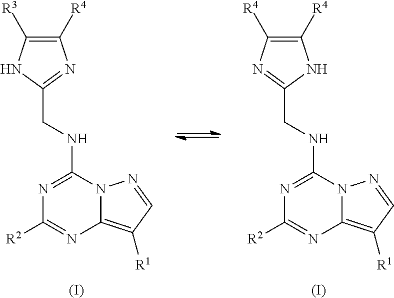

The present invention includes all possible tautomers of the compounds of formula (I) of the present invention where X is a CR⁴ group as single tautomers, or as any mixture of any two or more possible tautomers, in any ratio.

Further, in the context of the present invention, it may be possible for the triazine core of the compounds of formula (I) to exhibit tautomerism and for said compounds to exist as single tautomers or even as a mixture in any amount of two or more of the possible tautomers:

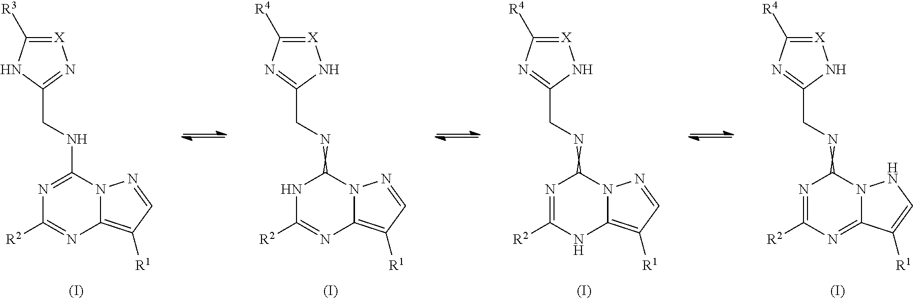

Further, the compounds of the present invention can exist as N-oxides, which are defined in that at least one nitrogen of the compounds of the present invention is oxidised. The present invention includes all such possible N-oxides.

The present invention also provides useful forms of the compounds of the present invention, such as metabolites, hydrates, solvates, prodrugs, salts, in particular pharmaceutically acceptable salts, and/or co-precipitates.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

Physiologically acceptable salts of the compounds according to the invention encompass acid addition salts of mineral acids, carboxylic acids and sulfonic acids, for example salts of hydrochloric acid, hydrobromic acid, hydroiodic acid, sulfuric acid, bisulfuric acid, phosphoric acid, nitric acid or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalenedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

A “pharmaceutically acceptable anion” refers to the deprotonated form of a conventional acid, such as, for example, a hydroxide, a carboxylate, a sulfate, a halide, a phosphate, or a nitrate.

Physiologically acceptable salts of the compounds according to the invention also comprise salts of conventional bases, such as, by way of example and by preference, alkali metal salts (for example lithium, sodium and potassium salts), alkaline earth metal salts (for example calcium, strontium and magnesium salts) and ammonium salts derived from ammonia or organic amines with 1 to 16 C atoms, such as, by way of example and by preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine, N-methylglucamine, dimethylglucamine, ethylglucamine, 1,6-hexadiamine, glucosamine, sarcosine, serinol, tris(hydroxymethyl)aminomethane, aminopropanediol, Sovak base, and 1-amino-2,3,4-butanetriol.

Additionally, the compounds according to the invention may form salts with a quaternary ammonium ion obtainable, e.g., by quaternisation of a basic nitrogen-containing group with agents such as lower alkylhalides such as methyl-, ethyl-, propyl-, and butylchlorides, -bromides and -iodides; dialkylsulfates such as dimethyl-, diethyl-, dibutyl- and diamylsulfates, long chain halides such as decyl-, lauryl-, myristyl- and stearylchlorides, -bromides and -iodides, aralkylhalides such as benzyl- and phenethylbromides and others. Examples of suitable quaternary ammonium ions are tetramethylammonium, tetraethylammonium, tetra(n-propyl)ammonium, tetra (n-butyl)ammonium, or N-benzyl-N,N,N-trimethylammonium.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF₃COOH”, “x Na⁺”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

Unless specified otherwise, suffixes to chemical names or structural formulae relating to salts, such as “hydrochloride”, “trifluoroacetate”, “sodium salt”, or “x HCl”, “x CF₃COOH”, “x Na+”, for example, mean a salt form, the stoichiometry of which salt form not being specified.

Solvates and hydrates of disclosed intermediates or example compounds, or salts thereof, which have been obtained, by the preparation and/or purification processes described herein, may be formed in any ratio.

Furthermore, the present invention includes all possible crystalline forms, or polymorphs, of the compounds of the present invention, either as a single polymorph, or as a mixture of more than one polymorph, in any ratio.

Moreover, the present invention also includes prodrugs of the compounds according to the invention. The term “prodrugs” designates compounds which themselves can be biologically active or inactive, but are converted (for example metabolically or hydrolytically) into compounds according to the invention during their residence time in the body. For example, a prodrug may be in the form of an in vivo hydrolysable ester of the specified compound. Derivatives of the compounds of formula (I) and the salts thereof which are converted into a compound of formula (I) or a salt thereof in a biological system (bioprecursors or pro-drugs) are covered by the invention. Said biological system may be, for example, a mammalian organism, particularly a human subject. The bioprecursor is, for example, converted into the compound of formula (I) or a salt thereof by metabolic processes.

Further, in the context of the present invention, when the inhibitory and/or degradatory activity of the compounds of formula (I) according to the present invention is referred to, the following terms are defined as follows:

As used herein and in the context of the present invention, the term “IC50 CDK12 hATP” refers to the IC₅₀ values obtained according to the assay described in section 2.2 of the Experimental Section herein below, i.e. the IC₅₀ values for the inhibition of CDK12 at high ATP.

As used herein and in the context of the present invention, the term “DC50 CDK12” refers to the DC₅₀ values obtained according to the assay described in section 7 of the Experimental Section herein below, i.e. the DC₅₀ values for the degradation of CDK12.

### DESCRIPTION

### Further Embodiments of the First Aspect of the Present Invention

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a heterocycloalkyl group and a —NR^{a}R^{b} group,
   wherein said heterocycloalkyl group is connected to the rest of the molecule via a carbon atom of said heterocycloalkyl group,
   wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
      wherein said C₃-C₈-cycloalkyl, phenyl, heteroaryl or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
   X is selected from a nitrogen atom and a CR⁴ group;
   R³ is selected from a C₃-C₈-cycloalkyl group, a heterocycloalkyl group, a phenyl group and a heteroaryl group,
      wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a ROOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R³ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₄-alkyl group, a C₁-C₃-haloalkyl group, a C₃-C₆-cycloalkyl group, a cyano group, a phenyl group, a heterocycloalkyl group and a heteroaryl group,
   wherein said C₁-C₄-alkyl, C₃-C₆-cycloalkyl, phenyl, heterocycloalkyl or heteroaryl group is each optionally substituted with one or more substituents independently selected from a halogen atom, a C₁-C₆-alkyl group, a C₁-C₆-alkoxy group, a C₁-C₆-haloalkyl group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a heterocycloalkyl group and a —NR^{a}R^{b} group,
   wherein said heterocycloalkyl group is connected to the rest of the molecule via a carbon atom of said heterocycloalkyl group,
   wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
      wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
      wherein said heterocycloalkyl, heterocycloalkenyl or heteroaryl group contains one or two heteroatoms independently selected from nitrogen, oxygen and sulfur,
      and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R^{a} is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₄-alkyl group, a C₁-C₃-haloalkyl group, a C₃-C₆-cycloalkyl group, a cyano group, a phenyl group, a heterocycloalkyl group and a heteroaryl group,
   wherein said C₁-C₄-alkyl, C₃-C₆-cycloalkyl, phenyl, heterocycloalkyl or heteroaryl group is each optionally substituted with one or more substituents independently selected from a halogen atom, a C₁-C₆-alkyl
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a heterocycloalkyl group and a —NR^{a}R^{b} group,
   wherein said heterocycloalkyl group is connected to the rest of the molecule via a carbon atom of said heterocycloalkyl group,
   wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
      wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₄-alkyl group, a C₁-C₃-haloalkyl group, a C₃-C₆-cycloalkyl group, a cyano group, a phenyl group, a heterocycloalkyl group and a heteroaryl group,
   wherein said C₁-C₄-alkyl, C₃-C₆-cycloalkyl, phenyl, heterocycloalkyl or heteroaryl group is each optionally substituted with one or more substituents independently selected from a halogen atom, a C₁-C₆-alkyl group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a (C₃-C₆-cycloalkyl)-(C₁-C₂-alkyl)-O— group, a (C₃-C₆-hydroxyalkyl)-(C₁-C₂-alkyl)-O— group, a (C₃-C₆-alkoxyalkyl)-(C₁-C₂-alkyl)-O— group, a ((CH₃)₂N)—(C₁-C₂-alkyl)-O— group, a heterocycloalkyl group, a (heterocycloalkyl)-O— group and a —NR^{a}R^{b} group,
   wherein said heterocycloalkyl group is connected to the rest of the molecule via a carbon atom of said heterocycloalkyl group,
   wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
      wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a heterocycloalkyl group and a —NR^{a}R^{b} group,
   wherein said heterocycloalkyl group is connected to the rest of the molecule via a carbon atom of said heterocycloalkyl group,
   wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
      wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R^{a} is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a heterocycloalkyl group and a —NR^{a}R^{b} group,
   wherein said heterocycloalkyl group is connected to the rest of the molecule via a carbon atom of said heterocycloalkyl group,
   wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
      wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a (C₃-C₆-cycloalkyl)-(C₁-C₂-alkyl)-O— group, a (C₃-C₆-hydroxyalkyl)-(C₁-C₂-alkyl)-O— group, a (C₃-C₆-alkoxyalkyl)-(C₁-C₂-alkyl)-O— group, a ((CH₃)₂N)—(C₁-C₂-alkyl)-O— group, a heterocycloalkyl group, a (heterocycloalkyl)-O— group and a —NR^{a}R^{b} group,
   wherein said heterocycloalkyl group is connected to the rest of the molecule via a carbon atom of said heterocycloalkyl group,
   wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
      wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a heterocycloalkyl group and a —NR^{a}R^{b} group,
   wherein said heterocycloalkyl group is connected to the rest of the molecule via a carbon atom of said heterocycloalkyl group,
   wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
      wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is a nitrogen atom;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a heterocycloalkyl group and a —NR^{a}R^{b} group,
   wherein said heterocycloalkyl group is connected to the rest of the molecule via a carbon atom of said heterocycloalkyl group,
   wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
      wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a heterocycloalkyl group and a —NR^{a}R^{b} group,
   wherein said heterocycloalkyl group is connected to the rest of the molecule via a carbon atom of said heterocycloalkyl group,
   wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
      wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is a CR⁴ group;
   and R³ and R⁴, together with the carbon atoms to which they are attached form a 6-membered cycloalkenyl group, a phenyl group or a 6-membered heteroaryl group,
   wherein said heteroaryl group contains one or two heteroatoms independently selected from nitrogen, oxygen and sulfur,
   and wherein said cycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a (C₃-C₆-cycloalkyl)-(C₁-C₂-alkyl)-O— group, a (C₃-C₆-hydroxyalkyl)-(C₁-C₂-alkyl)-O— group, a (C₃-C₆-alkoxyalkyl)-(C₁-C₂-alkyl)-O— group, a ((CH₃)₂N)—(C₁-C₂-alkyl)-O— group, a heterocycloalkyl group, a (heterocycloalkyl)-O— group and a —NR^{a}R^{b} group,
   wherein said heterocycloalkyl group is connected to the rest of the molecule via a carbon atom of said heterocycloalkyl group,
   wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
      wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group and a R⁵R⁶N— group, wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is a CR⁴ group;
   and R³ and R⁴, together with the carbon atoms to which they are attached form a 6-membered cycloalkenyl group, a phenyl group or a 6-membered heteroaryl group,
   wherein said heteroaryl group contains one or two heteroatoms independently selected from nitrogen, oxygen and sulfur,
   and wherein said cycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₆-cycloalkoxy group, a R⁵R⁶N— group, a (R⁵R⁶N)—(C₁-C₆-alkyl)- group, and a R⁷OOC— group;
   R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
   R⁸ is selected from a hydrogen atom, a C₁-C₆-alkyl group, a C₃-C₆-cycloalkyl group and a C₁-C₆-haloalkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkoxy group, a C₁-C₆-haloalkoxy group, a C₃-C₈-cycloalkoxy group, a (C₃-C₆-cycloalkyl)-(C₁-C₂-alkyl)-O— group, a (C₃-C₆-hydroxyalkyl)-(C₁-C₂-alkyl)-O— group, a (C₃-C₆-alkoxyalkyl)-(C₁-C₂-alkyl)-O— group, a ((CH₃)₂N)—(C₁-C₂-alkyl)-O— group and a (heterocycloalkyl)-O— group,
   wherein said heterocycloalkyl group is connected to the rest of the molecule via a carbon atom of said heterocycloalkyl group;
X is a CR⁴ group;
   and R³ and R⁴, together with the carbon atoms to which they are attached form a 6-membered cycloalkenyl group, a phenyl group or a 6-membered heteroaryl group,
   wherein said heteroaryl group contains one or two heteroatoms independently selected from nitrogen, oxygen and sulfur,
   and wherein said cycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R³ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group,
   a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a heterocycloalkyl group and a —NR^{a}R^{b} group,
   wherein said heterocycloalkyl group is connected to the rest of the molecule via a carbon atom of said heterocycloalkyl group,
   wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
      wherein said C₃-C₈-cycloalkyl, phenyl, heteroaryl or heterocycloalkyl group is each optionally substituted with one or more substituents independently selected from a halogen atom, a hydroxy group, a cyano group, a C₁-alkyl group, a C₁-hydroxyalkyl group, a C₁-haloalkyl group, a C₁-alkoxy group, a C₁-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a heterocycloalkyl group and a —NR^{a}R^{b} group,
   wherein said heterocycloalkyl group is connected to the rest of the molecule via a carbon atom of said heterocycloalkyl group,
   wherein R^{a} and R^{b} are each independently selected from a hydrogen atom, a C₁-C₆-alkyl group, a C₁-C₆-hydroxyalkyl group, a heteroaryl group and a phenyl group,
      wherein said phenyl, heteroaryl or heterocycloalkyl group is each optionally substituted with one or more substituents independently selected from a halogen atom, a cyano group, a C₁-alkyl group, a C₁-hydroxyalkyl group, a C₁-haloalkyl group, a C₁-alkoxy group, a C₁-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} are each independently selected from a hydrogen atom, a C₁-C₆-alkyl group, a C₁-C₆-hydroxyalkyl group, a heteroaryl group and a phenyl group,
      wherein said phenyl, heteroaryl or heterocycloalkyl group is each optionally substituted with one or more substituents independently selected from a halogen atom, a cyano group, a C₁-alkyl group, a C₁-hydroxyalkyl group, a C₁-haloalkyl group, a C₁-alkoxy group, a C₁-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a —NR^{a}R^{b} group,
   wherein R^{a} and R^{b} are each independently selected from a hydrogen atom, a C₁-C₆-alkyl group, a C₁-C₆-hydroxyalkyl group, a heteroaryl group and a phenyl group,
      wherein said phenyl, heteroaryl or heterocycloalkyl group is each optionally substituted with one or more substituents independently selected from a halogen atom, a cyano group, a C₁-alkyl group, a C₁-hydroxyalkyl group, a C₁-haloalkyl group, a C₁-alkoxy group, a C₁-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur, and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a nitrogen atom;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
   and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkoxy group, a C₁-C₆-haloalkoxy group, a C₃-C₈-cycloalkoxy group, a (C₃-C₆-cycloalkyl)-(C₁-C₂-alkyl)-O— group, a (C₃-C₆-hydroxyalkyl)-(C₁-C₂-alkyl)-O— group, a (C₃-C₆-alkoxyalkyl)-(C₁-C₂-alkyl)-O— group, a ((CH₃)₂N)—(C₁-C₂-alkyl)-O— group, a (heterocycloalkyl)-O— group and a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
   and R³ and R⁴, together with the carbon atoms to which they are attached form a 6-membered cycloalkenyl group, a phenyl group or a 6-membered heteroaryl group,
   wherein said heteroaryl group contains one or two heteroatoms independently selected from nitrogen, oxygen and sulfur,
   and wherein said cycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a 5- to 11-membered nitrogen-containing bicyclic heterocycloalkyl group, a 7- to 9-membered nitrogen-containing bridged compound or a 7- to 12-membered nitrogen-containing spiro compound,
      said 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur, and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound containing one, two or three further heteroatoms independently selected from, oxygen and sulfur or one group selected from —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group,
   a phenyl group, a heterocycloalkyl group and a heteroaryl group,
      wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group,
      said 4- to 9-membered nitrogen-containing monocyclic or 5- to 11-membered nitrogen-containing bicyclic heterocycloalkyl group containing one, two or three further heteroatoms independently selected from, oxygen and sulfur or one group selected from —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a morpholine ring,
      said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a morpholine ring,
      said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a nitrogen atom;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a morpholine ring,
      said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur, and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a morpholine ring,
      said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
   and R³ and R⁴, together with the carbon atoms to which they are attached form a 6-membered cycloalkenyl group, a phenyl group or a 6-membered heteroaryl group,
   wherein said heteroaryl group contains one or two heteroatoms independently selected from nitrogen, oxygen and sulfur,
   and wherein said cycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} are each independently selected from a hydrogen atom, a C₁-C₃-alkyl group, a C₃-C₆-cycloalkyl group, a C₁-C₃-haloalkyl group, a (C₃-C₆-cycloalkyl)-(C₁-C₃-alkyl)- group, a heterocycloalkyl group, a heteroaryl group and a phenyl group,
      wherein said C₃-C₆-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁a C₁-C₃-haloalkyl group, a C₁-C₃-alkoxy group, a C₁-C₃-haloalkoxy group and a R⁵R⁶N— group,
      wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      said 4- to 9-membered nitrogen-containing monocyclic, 5- to 11-membered nitrogen-containing bicyclic heterocycloalkyl group, 7- to 9-membered nitrogen-containing bridged compound or 7- to 12-membered nitrogen-containing spiro compound each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo,
      wherein when R^{a} and R^{b} together with the nitrogen atom to which they are attached form a pyrrolidine ring, a piperidine ring or a piperazine ring, said pyrrolidine, piperidine or piperazine ring is not substituted in any of its carbon atoms;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R^{a} is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} are each independently selected from a hydrogen atom, a C₁-C₃-alkyl group, a C₃-C₆-cycloalkyl group, a C₁-C₃-haloalkyl group, a (C₃-C₆-cycloalkyl)-(C₁-C₃-alkyl)- group, a heterocycloalkyl group, a heteroaryl group and a phenyl group,
      wherein said C₃-C₆-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁a C₁-C₃-haloalkyl group, a C₁-C₃-alkoxy group, a C₁-C₃-haloalkoxy group and a R⁵R⁶N— group,
      wherein at least one of R^{a} or R^{b} is not a hydrogen atom;
   or R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group, a 7- to 9-membered nitrogen-containing bridged compound or a 7- to 12-membered nitrogen-containing spiro compound,
      said or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      said azetidine ring, morpholine ring, 5- to 11-membered nitrogen-containing bicyclic heterocycloalkyl group, 7- to 9-membered nitrogen-containing bridged compound or 7- to 12-membered nitrogen-containing spiro compound each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} are each independently selected from a hydrogen atom, a C₁-C₃-alkyl group, a C₃-C₆-cycloalkyl group, a C₁-C₃-haloalkyl group, a (C₃-C₆-cycloalkyl)-(C₁-C₃-alkyl)- group, a heterocycloalkyl group, a heteroaryl group and a phenyl group,
      wherein said C₃-C₆-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁a C₁-C₃-haloalkyl group, a C₁-C₃-alkoxy group, a C₁-C₃-haloalkoxy group and a R⁵R⁶N— group,
      wherein at least one of R^{a} or R^{b} is not a hydrogen atom;
   or R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group, a 7- to 9-membered nitrogen-containing bridged compound or a 7- to 12-membered nitrogen-containing spiro compound,
      said or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      said azetidine ring, morpholine ring, 5- to 11-membered nitrogen-containing bicyclic heterocycloalkyl group, 7- to 9-membered nitrogen-containing bridged compound or 7- to 12-membered nitrogen-containing spiro compound each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a nitrogen atom;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a
R⁵R⁶N— group, and a R⁷OOC— group; R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} are each independently selected from a hydrogen atom, a C₁-C₃-alkyl group, a C₃-C₆-cycloalkyl group, a C₁-C₃-haloalkyl group, a (C₃-C₆-cycloalkyl)-(C₁-C₃-alkyl)- group, a heterocycloalkyl group, a heteroaryl group and a phenyl group,
      wherein said C₃-C₆-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁a C₁-C₃-haloalkyl group, a C₁-C₃-alkoxy group, a C₁-C₃-haloalkoxy group and a R⁵R⁶N— group,
      wherein at least one of R^{a} or R^{b} is not a hydrogen atom;
   or R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group, a 7- to 9-membered nitrogen-containing bridged compound or a 7- to 12-membered nitrogen-containing spiro compound,
      said or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      said azetidine ring, morpholine ring, 5- to 11-membered nitrogen-containing bicyclic heterocycloalkyl group, 7- to 9-membered nitrogen-containing bridged compound or 7- to 12-membered nitrogen-containing spiro compound each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} are each independently selected from a hydrogen atom, a C₁-C₃-alkyl group, a C₃-C₆-cycloalkyl group, a C₁-C₃-haloalkyl group, a (C₃-C₆-cycloalkyl)-(C₁-C₃-alkyl)- group, a heterocycloalkyl group, a heteroaryl group and a phenyl group,
      wherein said C₃-C₆-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁a C₁-C₃-haloalkyl group, a C₁-C₃-alkoxy group, a C₁-C₃-haloalkoxy group and a R⁵R⁶N— group,
      wherein at least one of R^{a} or R^{b} is not a hydrogen atom;
   or R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group, a 7- to 9-membered nitrogen-containing bridged compound or a 7- to 12-membered nitrogen-containing spiro compound,
      said or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      said azetidine ring, morpholine ring, 5- to 11-membered nitrogen-containing bicyclic heterocycloalkyl group, 7- to 9-membered nitrogen-containing bridged compound or 7- to 12-membered nitrogen-containing spiro compound each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
   and R³ and R⁴, together with the carbon atoms to which they are attached form a 6-membered cycloalkenyl group, a phenyl group or a 6-membered heteroaryl group,
   wherein said heteroaryl group contains one or two heteroatoms independently selected from nitrogen, oxygen and sulfur,
   and wherein said cycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} are each independently selected from a hydrogen atom, a C₁-C₃-alkyl group, a C₃-C₆-cycloalkyl group, a C₁-C₃-haloalkyl group, a (C₃-C₆-cycloalkyl)-(C₁-C₃-alkyl)- group, a heterocycloalkyl group, a heteroaryl group and a phenyl group,
      wherein said C₃-C₆-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁a C₁-C₃-haloalkyl group, a C₁-C₃-alkoxy group, a C₁-C₃-haloalkoxy group and a R⁵R⁶N— group,
      wherein at least one of R^{a} or R^{b} is not a hydrogen atom;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} together with the nitrogen atom to which they are attached form an azetidine ring, a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic, 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      said 4- to 9-membered nitrogen-containing monocyclic, 5- to 11-membered nitrogen-containing bicyclic heterocycloalkyl group, 7- to 9-membered nitrogen-containing bridged compound or 7- to 12-membered nitrogen-containing spiro compound each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo,
      wherein when R^{a} and R^{b} together with the nitrogen atom to which they are attached form a pyrrolidine ring, a piperidine ring or a piperazine ring, said pyrrolidine, piperidine or piperazine ring is not substituted in any of its carbon atoms;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group, a 7- to 9-membered nitrogen-containing bridged compound or a 7- to 12-membered nitrogen-containing spiro compound,
      said 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      said azetidine ring, morpholine ring, 5- to 11-membered nitrogen-containing bicyclic heterocycloalkyl group, 7- to 9-membered nitrogen-containing bridged compound or 7- to 12-membered nitrogen-containing spiro compound each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur, and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} together with the nitrogen atom to which they are attached form an azetidine ring, a 4- to 9-membered nitrogen-containing monocyclic or 5- to 11-membered nitrogen-containing bicyclic heterocycloalkyl group,
      said 4- to 9-membered nitrogen-containing monocyclic or 5- to 11-membered nitrogen-containing bicyclic heterocycloalkyl group each optionally containing one, two or three further heteroatoms independently selected from nitrogen, oxygen and sulfur or one group selected from —NR⁸—, —S(═O)—, —S(═O)₂— and —S(═O)(═NH)—,
      said 4- to 9-membered nitrogen-containing monocyclic or 5- to 11-membered nitrogen-containing bicyclic heterocycloalkyl group each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo,
      wherein when R^{a} and R^{b} together with the nitrogen atom to which they are attached form a pyrrolidine ring, a piperidine ring or a piperazine ring, said pyrrolidine, piperidine or piperazine ring is not substituted in any of its carbon atoms;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group,
      said 5- to 11-membered nitrogen-containing bicyclic heterocycloalkyl group each optionally containing one, two or three further heteroatoms independently selected from nitrogen, oxygen and sulfur or one group selected from —NR⁸—, —S(═O)—, —S(═O)₂— and —S(═O)(═NH)—,
      said azetidine ring, morpholine ring or 5- to 11-membered nitrogen-containing bicyclic heterocycloalkyl group each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group,
      said 5- to 11-membered nitrogen-containing bicyclic heterocycloalkyl group each optionally containing one, two or three further heteroatoms independently selected from nitrogen, oxygen and sulfur or one group selected from —NR⁸—, —S(═O)—, —S(═O)₂— and —S(═O)(═NH)—,
      said azetidine ring, morpholine ring or 5- to 11-membered nitrogen-containing bicyclic heterocycloalkyl group each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a nitrogen atom;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group,
      said 5- to 11-membered nitrogen-containing bicyclic heterocycloalkyl group each optionally containing one, two or three further heteroatoms independently selected from nitrogen, oxygen and sulfur or one group selected from —NR⁸—, —S(═O)—, —S(═O)₂— and —S(═O)(═NH)—,
      said azetidine ring, morpholine ring or 5- to 11-membered nitrogen-containing bicyclic heterocycloalkyl group each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is selected from a C₁-C₆-alkoxy group, a C₁-C₆-haloalkoxy group, a C₃—C-cycloalkoxy group, a (C₃-C₆-cycloalkyl)-(C₁-C₂-alkyl)-O— group, a (C₃-C₆-hydroxyalkyl)-(C₁-C₂-alkyl)-O— group, a (C₃-C₆-alkoxyalkyl)-(C₁-C₂-alkyl)-O— group, a ((CH₃)₂N)—(C₁-C₂-alkyl)-O— group, a (heterocycloalkyl)-O— group and a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring, a morpholine ring or a 5- to 11-membered nitrogen-containing bicyclic heterocycloalkyl group,
      said 5- to 11-membered nitrogen-containing bicyclic heterocycloalkyl group each optionally containing one, two or three further heteroatoms independently selected from nitrogen, oxygen and sulfur or one group selected from —NR⁸—, —S(═O)—, —S(═O)₂— and —S(═O)(═NH)—,
      said azetidine ring, morpholine ring or 5- to 11-membered nitrogen-containing bicyclic heterocycloalkyl group each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group,
      said 5- to 11-membered nitrogen-containing bicyclic heterocycloalkyl group each optionally containing one, two or three further heteroatoms independently selected from nitrogen, oxygen and sulfur or one group selected from —NR⁸—, —S(═O)—, —S(═O)₂— and —S(═O)(═NH)—,
      said azetidine ring, morpholine ring or 5- to 11-membered nitrogen-containing bicyclic heterocycloalkyl group each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
   and R³ and R⁴, together with the carbon atoms to which they are attached form a 6-membered cycloalkenyl group, a phenyl group or a 6-membered heteroaryl group,
   wherein said heteroaryl group contains one or two heteroatoms independently selected from nitrogen, oxygen and sulfur,
   and wherein said cycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
   R⁷ is selected from a hydrogen atom and a C₁-C₃-alkyl group;
   R⁸ is selected from a hydrogen atom, a C₁-C₆-alkyl group, a C₃-C₆-cycloalkyl group and a C₁-C₆-haloalkyl group;
      or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} together with the nitrogen atom to which they are attached form a 4- to 9-membered nitrogen-containing monocyclic heterocycloalkyl group,
      said 4- to 9-membered nitrogen-containing monocyclic heterocycloalkyl group each optionally containing one, two or three further heteroatoms independently selected from nitrogen, oxygen and sulfur or one group selected from —NR⁸—, —S(═O)—, —S(═O)₂— and —S(═O)(═NH)—,
      said 4- to 9-membered nitrogen-containing monocyclic heterocycloalkyl group each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo,
      wherein when R^{a} and R^{b} together with the nitrogen atom to which they are attached form a pyrrolidine ring, a piperidine ring or a piperazine ring, said pyrrolidine, piperidine or piperazine ring is not substituted in any of its carbon atoms;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring or a morpholine ring,
      said azetidine ring or morpholine ring each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring or a morpholine ring,
      said azetidine ring or morpholine ring each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a nitrogen atom;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁵ and R⁶ are each independently selected from a hydrogen atom, a C₁-C₆-alkyl group,
a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring or a morpholine ring,
      said azetidine ring or morpholine ring each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring or a morpholine ring,
      said azetidine ring or morpholine ring each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
   and R³ and R⁴, together with the carbon atoms to which they are attached form a 6-membered cycloalkenyl group, a phenyl group or a 6-membered heteroaryl group,
   wherein said heteroaryl group contains one or two heteroatoms independently selected from nitrogen, oxygen and sulfur,
   and wherein said cycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group,
R² is a —NR^{a}R^{b} group,
   wherein R^{a} and R^{b} together with the nitrogen atom to which they are attached form an azetidine ring or a morpholine ring,
      said azetidine ring or morpholine ring each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, R³ and R⁴, together with the carbon atoms to which they are attached form a 6-membered cycloalkenyl group, a phenyl group or a 6-membered heteroaryl group,
   wherein said heteroaryl group contains one or two heteroatoms independently selected from nitrogen, oxygen and sulfur,
   and wherein said cycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a C₁-C₆-alkyl group, a C₁-C₆-haloalkyl group, a C₃-C₈-cycloalkyl group, a C₃-C₈-halocycloalkyl group and a (C₃-C₈-cycloalkyl)-(C₁-C₆-alkyl)- group;
R² is a —NR^{a}R^{b} group,
   wherein R^{a} and R^{b} together with the nitrogen atom to which they are attached form a morpholine ring, a 5- to 11-membered nitrogen-containing bicyclic heterocycloalkyl group, a 7- to 9-membered nitrogen-containing bridged compound or a 7- to 12-membered nitrogen-containing spiro compound,
      said 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
      said morpholine ring, 5- to 11-membered nitrogen-containing bicyclic heterocycloalkyl group, 7- to 9-membered nitrogen-containing bridged compound or 7- to 12-membered nitrogen-containing spiro compound each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a C₁-C₆-alkyl group, a C₁-C₆-haloalkyl group, a C₃-C₈-cycloalkyl group, a C₃-C₈-halocycloalkyl group and a (C₃-C₈-cycloalkyl)-(C₁-C₆-alkyl)- group;
R² is a —NR^{a}R^{b} group,
   wherein R^{a} and R^{b} together with the nitrogen atom to which they are attached form a morpholine ring,
      said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound containing one, two or three further heteroatoms independently selected from, oxygen and sulfur or one group selected from —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is preferably substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a C₁-C₆-alkyl group, a C₁-C₆-haloalkyl group, a C₃-C₈-cycloalkyl group, a C₃-C₈-halocycloalkyl group and a (C₃-C₈-cycloalkyl)-(C₁-C₆-alkyl)- group;
R² is a —NR^{a}R^{b} group,
   wherein R^{a} and R^{b} together with the nitrogen atom to which they are attached form a morpholine ring,
      said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a nitrogen atom;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a C₁-C₆-alkyl group, a C₁-C₆-haloalkyl group, a C₃-C₈-cycloalkyl group, a C₃-C₈-halocycloalkyl group and a (C₃-C₈-cycloalkyl)-(C₁-C₆-alkyl)- group;
R² is a —NR^{a}R^{b} group,
   wherein R^{a} and R^{b} together with the nitrogen atom to which they are attached form a morpholine ring,
      said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
R³ is selected from a C₃-C₈-cycloalkyl group, a heterocycloalkyl group, a phenyl group and a heteroaryl group,
   wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
   or R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a C₁-C₆-alkyl group, a C₁-C₆-haloalkyl group, a C₃-C₈-cycloalkyl group, a C₃-C₈-halocycloalkyl group and a (C₃-C₈-cycloalkyl)-(C₁-C₆-alkyl)- group;
R² is a —NR^{a}R^{b} group,
   wherein R^{a} and R^{b} together with the nitrogen atom to which they are attached form a morpholine ring,
      said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is a CR⁴ group;
   and R³ and R⁴, together with the carbon atoms to which they are attached form a 6-membered cycloalkenyl group, a phenyl group or a 6-membered heteroaryl group,
   wherein said heteroaryl group contains one or two heteroatoms independently selected from nitrogen, oxygen and sulfur,
   and wherein said cycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound containing one, two or three further heteroatoms independently selected from, oxygen and sulfur or one group selected from —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is preferably substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group,
a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound containing one, two or three further heteroatoms independently selected from, oxygen and sulfur or one group selected from —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is preferably substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur, and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound containing one, two or three further heteroatoms independently selected from, oxygen and sulfur or one group selected from —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is preferably substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group,
a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound containing one, two or three further heteroatoms independently selected from, oxygen and sulfur or one group selected from —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a morpholine ring,
      said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group, and wherein, when substituted, said phenyl group is preferably substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a morpholine ring,
      said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group; R⁵ and R⁶ are each independently selected from a hydrogen atom, a C₁-C₆-alkyl group,
a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group;
R² is a —NR^{a}R^{b} group,
   wherein R^{a} and R^{b} are each independently selected from a hydrogen atom, a C₁-C₃-alkyl group, a C₃-C₆-cycloalkyl group, a C₁-C₃-haloalkyl group, a (C₃-C₆-cycloalkyl)-(C₁-C₃-alkyl)- group, a heterocycloalkyl group, a heteroaryl group and a phenyl group,
      wherein said C₃-C₆-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁a C₁-C₃-haloalkyl group, a C₁-C₃-alkoxy group, a C₁-C₃-haloalkoxy group and a R⁵R⁶N— group, wherein at least one of R^{a} or R^{b} is not a hydrogen atom;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is preferably substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group;
R² is a —NR^{a}R^{b} group,
   wherein R^{a} and R^{b} are each independently selected from a hydrogen atom, a C₁-C₃-alkyl group, a C₃-C₆-cycloalkyl group, a C₁-C₃-haloalkyl group, a (C₃-C₆-cycloalkyl)-(C₁-C₃-alkyl)- group, a heterocycloalkyl group, a heteroaryl group and a phenyl group,
      wherein said C₃-C₆-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁a C₁-C₃-haloalkyl group, a C₁-C₃-alkoxy group, a C₁-C₃-haloalkoxy group and a R⁵R⁶N— group
      wherein at least one of R^{a} or R^{b} is not a hydrogen atom;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group,
      said 4- to 9-membered nitrogen-containing monocyclic or 5- to 11-membered nitrogen-containing bicyclic heterocycloalkyl group each optionally containing one, two or three further heteroatoms independently selected from nitrogen, oxygen and sulfur or one group selected from —NR⁸—, —S(═O)—, —S(═O)₂— and —S(═O)(═NH)—
      said 4- to 9-membered nitrogen-containing monocyclic or 5- to 11-membered nitrogen-containing bicyclic heterocycloalkyl group each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo,
      wherein when R^{a} and R^{b} together with the nitrogen atom to which they are attached form a pyrrolidine ring, a piperidine ring or a piperazine ring, said pyrrolidine, piperidine or piperazine ring is not substituted in any of its carbon atoms;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is preferably substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group; R⁷ is selected from a hydrogen atom and a C₁-C₃-alkyl group; R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.
      In accordance with further embodiments, the present invention provides compounds of general formula (I), supra, wherein: R¹ is selected from a halogen atom and a cyano group; R² is a —NR^{a}R^{b} group, wherein R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group, said 5- to 11-membered nitrogen-containing bicyclic heterocycloalkyl group each optionally containing one, two or three further heteroatoms independently selected from nitrogen, oxygen and sulfur or one group selected from —NR⁸—, —S(═O)—, —S(═O)₂— and —S(═O)(═NH)—, wherein said azetidine ring, morpholine ring, 5- to 11-membered nitrogen-containing bicyclic heterocycloalkyl group each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo; X is selected from a nitrogen atom and a CR⁴ group; R³ is selected from a phenyl group and a heteroaryl group, wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group, and wherein, when substituted, said phenyl group is preferably substituted in one or more of the ortho- and/or meta-positions with respect to the point of attachment of said phenyl group to the rest of the molecule; R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group, wherein said heterocycloalkyl, heterocycloalkenyl or heteroaryl group contains one or two heteroatoms independently selected from nitrogen, oxygen and sulfur, and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R^{a} is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group,
      said 4- to 9-membered nitrogen-containing monocyclic or 5- to 11-membered nitrogen-containing bicyclic heterocycloalkyl group each optionally containing one, two or three further heteroatoms independently selected from nitrogen, oxygen and sulfur or one group selected from —NR⁸—, —S(═O)—, —S(═O)₂— and —S(═O)(═NH)—,
      said 4- to 9-membered nitrogen-containing monocyclic or 5- to 11-membered nitrogen-containing bicyclic heterocycloalkyl group each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
      wherein when R^{a} and R^{b} together with the nitrogen atom to which they are attached form a pyrrolidine ring, a piperidine ring or a piperazine ring, said pyrrolidine, piperidine or piperazine ring is not substituted in any of its carbon atoms;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group;
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group,
      said 5- to 11-membered nitrogen-containing bicyclic heterocycloalkyl group optionally containing one, two or three further heteroatoms independently selected from nitrogen, oxygen and sulfur or one group selected from —NR⁸—, —S(═O)—, —S(═O)₂— and —S(═O)(═NH)—,
      said azetidine ring, morpholine ring, 5- to 11-membered nitrogen-containing bicyclic heterocycloalkyl group optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group;
R² is a —NR^{a}R^{b} group,
   wherein R^{a} and R^{b} together with the nitrogen atom to which they are attached form a 4- to 9-membered nitrogen-containing monocyclic heterocycloalkyl group,
      said 4- to 9-membered nitrogen-containing monocyclic heterocycloalkyl group optionally containing one, two or three further heteroatoms independently selected from nitrogen, oxygen and sulfur or one group selected from —NR⁸—, —S(═O)—, —S(═O)₂— and —S(═O)(═NH)—,
      said 4- to 9-membered nitrogen-containing monocyclic heterocycloalkyl group optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo,
      wherein when R^{a} and R^{b} together with the nitrogen atom to which they are attached form a pyrrolidine ring, a piperidine ring or a piperazine ring, said pyrrolidine, piperidine or piperazine ring is not substituted in any of its carbon atoms;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is preferably substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R^{a} is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group;
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring, a morpholine ring,
      said azetidine ring or morpholine ring each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is preferably substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group;
R² is a —NR^{a}R^{b} group,
   wherein R^{a} and R^{b} together with the nitrogen atom to which they are attached form a 4- to 9-membered nitrogen-containing monocyclic heterocycloalkyl group,
      said 4- to 9-membered nitrogen-containing monocyclic heterocycloalkyl group optionally containing one, two or three further heteroatoms independently selected from nitrogen, oxygen and sulfur or one group selected from —NR⁸—, —S(═O)—, —S(═O)₂— and —S(═O)(═NH)—,
      said 4- to 9-membered nitrogen-containing monocyclic heterocycloalkyl group optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo,
      wherein when R^{a} and R^{b} together with the nitrogen atom to which they are attached form a pyrrolidine ring, a piperidine ring or a piperazine ring, said pyrrolidine, piperidine or piperazine ring is not substituted in any of its carbon atoms;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group;
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring or a morpholine ring,
      said azetidine ring or morpholine ring each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a C₁-C₆-alkyl group, a C₁-C₆-haloalkyl group, a C₃-C₈-cycloalkyl group, a C₃-C₈-halocycloalkyl group and a (C₃-C₈-cycloalkyl)-(C₁-C₆-alkyl)- group;
R² is a —NR^{a}R^{b} group,
   wherein R^{a} and R^{b} together with the nitrogen atom to which they are attached form an azetidine ring or a morpholine ring,
      said azetidine ring or morpholine ring each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is preferably substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group,
a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a C₁-C₆-alkyl group, a C₁-C₆-haloalkyl group, a C₃-C₈-cycloalkyl group, a C₃-C₈-halocycloalkyl group, a (C₃-C₈-cycloalkyl)-(C₁-C₆-alkyl)- group,
R² is a —NR^{a}R^{b} group,
   wherein R^{a} and R^{b} together with the nitrogen atom to which they are attached form a morpholine ring,
      said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
   wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound containing one, two or three further heteroatoms independently selected from, oxygen and sulfur or one group selected from —S(═O)—, —S(═O)₂— and —S(═O)(═NH)—,
      and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group, and wherein, when substituted, said phenyl group is substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group, a C₃-C₆-cycloalkyl group, a C₃-C₆-cycloalkoxy group, a R⁵R⁶N— group, a (R⁵R⁶N)—(C₁-C₆-alkyl)- group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group;
R² is a —NR^{a}R^{b} group,
   wherein R^{a} and R^{b} together with the nitrogen atom to which they are attached form a 4- to 9-membered nitrogen-containing monocyclic or 5- to 11-membered nitrogen-containing bicyclic heterocycloalkyl group,
      said 4- to 9-membered nitrogen-containing monocyclic or 5- to 11-membered nitrogen-containing bicyclic heterocycloalkyl group each optionally containing one, two or three further heteroatoms independently selected from nitrogen, oxygen and sulfur or one group selected from —NR⁸—, —S(═O)—, —S(═O)₂— and —S(═O)(═NH)—,
      wherein said 4- to 9-membered nitrogen-containing monocyclic or 5- to 11-membered nitrogen-containing bicyclic heterocycloalkyl group each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo,
      wherein when R^{a} and R^{b} together with the nitrogen atom to which they are attached form a pyrrolidine ring, a piperidine ring or a piperazine ring, said pyrrolidine, piperidine or piperazine ring is not substituted in any of its carbon atoms;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is substituted in one or more of the ortho- and/or meta-positions with respect to the point of attachment of said phenyl group to the rest of the molecule;
R⁴ is selected from a hydrogen atom, methyl group and a trifluoromethyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group, a C₃-C₆-cycloalkyl group, a C₃-C₆-cycloalkoxy group, a R⁵R⁶N— group, a (R⁵R⁶N)—(C₁-C₆-alkyl)- group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a halogen atom and a cyano group;
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group,
      said 5- to 11-membered nitrogen-containing bicyclic heterocycloalkyl group optionally containing one, two or three further heteroatoms independently selected from nitrogen, oxygen and sulfur or one group selected from —NR⁸—, —S(═O)—, —S(═O)₂— and —S(═O)(═NH)—,
      said azetidine ring, morpholine ring or 5- to 11-membered nitrogen-containing bicyclic heterocycloalkyl group each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is substituted in one or more of the ortho- and/or meta-positions with respect to the point of attachment of said phenyl group to the rest of the molecule;
R⁴ is selected from a hydrogen atom, methyl group and a trifluoromethyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group, a C₃-C₆-cycloalkyl group, a C₃-C₆-cycloalkoxy group, a R⁵R⁶N— group, a (R⁵R⁶N)—(C₁-C₆-alkyl)- group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
   or a tautomer, or an N-oxide, or a salt thereof, or a salt of a tautomer, or a salt of an N-oxide, or a mixture of same.

In accordance with further embodiments, the present invention provides compounds of general formula (I), supra, wherein:
R¹ is selected from a C₁-C₆-alkyl group, a C₁-C₆-haloalkyl group, a C₃-C₈-cycloalkyl group, a C₃-C₈-halocycloalkyl group and a (C₃-C₈-cycloalkyl)-(C₁-C₆-alkyl)- group;
R² is a —NR^{a}R^{b} group,
   wherein R^{a} and R^{b} together with the nitrogen atom to which they are attached form a morpholine ring,
      said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
   wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
   and wherein, when substituted, said phenyl group is substituted in one or more of the ortho- and/or meta-positions with respect to the point of attachment of said phenyl group to the rest of the molecule;
R⁴ is selected from a hydrogen atom, a methyl group and a trifluoromethyl group;
   or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
   wherein said heterocycloalkyl, heterocycloalkenyl or heteroaryl group contains one or two heteroatoms independently selected from nitrogen, oxygen and sulfur,
   and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group, a C₃-C₆-cycloalkyl group, a C₃-C₆-cycloalkoxy group, a R⁵R⁶N— group, a (R⁵R⁶N)—(C₁-C₆-alkyl)- group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heteroaryl group and a phenyl group;
R⁷ is selected from a hydrogen atom and a C₁-C₃-alkyl group;
   or a tautomer, or an N-oxide, or a salt thereof, or a salt of a tautomer, or a salt of an N-oxide, or a mixture of same.

The present invention provides the compounds of general formula (I) which are disclosed in the Example Section of this text, infra.

In some embodiments, the present invention includes compounds of general formula (I) selected from:
N-[(1H-benzimidazol-2-yl)methyl]-8-bromo-2-(morpholin-4-yl)pyrazolo[1,5-a][1,3,5]triazin-4-amine,
8-bromo-N-[(7-methyl-3H-imidazo[4,5-b]pyridin-2-yl)methyl]-2-(morpholin-4-yl)pyrazolo[1,5-a][1,3,5]triazin-4-amine,
8-bromo-2-(morpholin-4-yl)-N-[(5-phenyl-4H-1,2,4-triazol-3-yl)methyl]pyrazolo[1,5-a][1,3,5]triazin-4-amine,
8-bromo-N-{[5-(3-fluorophenyl)-4H-1,2,4-triazol-3-yl]methyl}-2-(morpholin-4-yl)pyrazolo[1,5-a][1,3,5]triazin-4-amine,
N-[(1H-benzimidazol-2-yl)methyl]-8-cyclopropyl-2-(morpholin-4-yl)pyrazolo[1,5-a][1,3,5]triazin-4-amine,
8-bromo-N-[(4-fluoro-1H-benzimidazol-2-yl)methyl]-2-(morpholin-4-yl)pyrazolo[1,5-a][1,3,5]triazin-4-amine,
8-bromo-2-[(2R,6S)-2,6-dimethylmorpholin-4-yl]-N-[(4-fluoro-1H-benzimidazol-2-yl)methyl]pyrazolo[1,5-a][1,3,5]triazin-4-amine,
8-bromo-N-[(4-fluoro-1H-benzimidazol-2-yl)methyl]-2-(4-methylpiperazin-1-yl)pyrazolo[1,5-a][1,3,5]triazin-4-amine,
8-bromo-N-[(4-methyl-1H-benzimidazol-2-yl)methyl]-2-(morpholin-4-yl)pyrazolo[1,5-a][1,3,5]triazin-4-amine,
8-bromo-N-[(5-methoxy-1H-benzimidazol-2-yl)methyl]-2-(morpholin-4-yl)pyrazolo[1,5-a][1,3,5]triazin-4-amine,
8-cyclopropyl-N-[(4,5-difluoro-1H-benzimidazol-2-yl)methyl]-2-(morpholin-4-yl)pyrazolo[1,5-a][1,3,5]triazin-4-amine,
8-cyclopropyl-N-[(4,5-difluoro-1H-benzimidazol-2-yl)methyl]-2-(piperazin-1-yl)pyrazolo[1,5-a][1,3,5]triazin-4-amine,
N-[(1H-benzimidazol-2-yl)methyl]-8-bromo-2-(4,7-diazaspiro[2.5]octan-7-yl)pyrazolo[1,5-a][1,3,5]triazin-4-amine,
8-cyclopropyl-2-[(3R,5S)-3,5-dimethylpiperazin-1-yl]-N-[(4-fluoro-1H-benzimidazol-2-yl)methyl]pyrazolo[1,5-a][1,3,5]triazin-4-amine,
8-cyclopropyl-N-[(4-fluoro-1H-benzimidazol-2-yl)methyl]-2-(morpholin-4-yl)pyrazolo[1,5-a][1,3,5]triazin-4-amine,
8-cyclopropyl-N-[(4-methoxy-1H-benzimidazol-2-yl)methyl]-2-(4-methylpiperazin-1-yl)pyrazolo[1,5-a][1,3,5]triazin-4-amine,
8-cyclopropyl-N-[(4-methoxy-1H-benzimidazol-2-yl)methyl]-2-morpholino-pyrazolo[1,5-a][1,3,5]triazin-4-amine,
8-cyclopropyl-N-[(4-methoxy-1H-benzimidazol-2-yl)methyl]-2-[(4aS,7aS)-octahydro-6H-pyrrolo[3,4-b]pyridin-6-yl]pyrazolo[1,5-a][1,3,5]triazin-4-amine,
N-(1H-benzimidazol-2-ylmethyl)-8-bromo-2-(2,5-diazabicyclo[2.2.2]octan-2-yl)pyrazolo[1,5-a][1,3,5]triazin-4-amine,
1-[(4-{[(1H-benzimidazol-2-yl)methyl]amino}-8-bromopyrazolo[1,5-a][1,3,5]triazin-2-yl)(methyl)amino]-2-methylpropan-2-ol,
N-[(1H-benzimidazol-2-yl)methyl]-2-(morpholin-4-yl)-8-(trifluoromethyl)pyrazolo[1,5-a][1,3,5]triazin-4-amine,
2-(4-aminopiperidin-1-yl)-N-[(1H-benzimidazol-2-yl)methyl]-8-bromopyrazolo[1,5-a][1,3,5]triazin-4-amine,
N-[(5-methoxy-1H-benzimidazol-2-yl)methyl]-2-(morpholin-4-yl)-8-(trifluoromethyl)pyrazolo[1,5-a][1,3,5]triazin-4-amine,
N-(1H-benzimidazol-2-ylmethyl)-8-bromo-2-[(2R or S)-2,4-dimethylpiperazin-1-yl]pyrazolo[1,5-a][1,3,5]triazin-4-amine,
N-[(1H-benzimidazol-2-yl)methyl]-8-bromo-2-[4-(methylamino)piperidin-1-yl]pyrazolo[1,5-a][1,3,5]triazin-4-amine,
N-[(5-fluoro-1H-benzimidazol-2-yl)methyl]-2-(morpholin-4-yl)-8-(trifluoromethyl)pyrazolo[1,5-a][1,3,5]triazin-4-amine,
N-[(4-fluoro-1H-benzimidazol-2-yl)methyl]-2-(morpholin-4-yl)-8-(trifluoromethyl)pyrazolo[1,5-a][1,3,5]triazin-4-amine,
N-[(5-chloro-1H-benzimidazol-2-yl)methyl]-2-(morpholin-4-yl)-8-(trifluoromethyl)pyrazolo[1,5-a][1,3,5]triazin-4-amine,
2-[(1R,5S,6s)-6-amino-3-azabicyclo[3.1.1]heptan-3-yl]-N-[(1H-benzimidazol-2-yl)methyl]-8-bromopyrazolo[1,5-a][1,3,5]triazin-4-amine,
1-[(8-cyclopropyl-4-{[(4,5-difluoro-1H-benzimidazol-2-yl)methyl]amino}pyrazolo[1,5-a][1,3,5]triazin-2-yl)(methyl)amino]-2-methylpropan-2-ol,
N-[(4-fluoro-1H-benzimidazol-2-yl)methyl]-2-(4-methylpiperazin-1-yl)-8-(trifluoromethyl)pyrazolo[1,5-a][1,3,5]triazin-4-amine,
8-cyclopropyl-2-[(1R,5S)-3,8-diazabicyclo[3.2.1]octan-3-yl]-N-[(4,5-difluoro-1H-benzimidazol-2-yl)methyl]pyrazolo[1,5-a][1,3,5]triazin-4-amine,
1-[(8-cyclopropyl-4-{[(4-fluoro-1H-benzimidazol-2-yl)methyl]amino}pyrazolo[1,5-a][1,3,5]triazin-2-yl)(methyl)amino]-2-methylpropan-2-ol,
8-cyclopropyl-2-[(1R,5S)-3,8-diazabicyclo[3.2.1]octan-3-yl]-N-[(4-fluoro-1H-benzimidazol-2-yl)methyl]pyrazolo[1,5-a][1,3,5]triazin-4-amine,
N-[(5-chloro-1H-benzimidazol-2-yl)methyl]-2-(4-methylpiperazin-1-yl)-8-(trifluoromethyl)pyrazolo[1,5-a][1,3,5]triazin-4-amine,
2-(3,8-diazabicyclo[3.2.1]octan-3-yl)-N-[(4,5-difluoro-1H-benzimidazol-2-yl)methyl]-8-(trifluoromethyl)pyrazolo[1,5-a][1,3,5]triazin-4-amine,
N-[(5,6-dichloro-1H-benzimidazol-2-yl)methyl]-2-(morpholin-4-yl)-8-(trifluoromethyl)pyrazolo[1,5-a][1,3,5]triazin-4-amine,
N-[(5,6-difluoro-1H-benzimidazol-2-yl)methyl]-2-(morpholin-4-yl)-8-(trifluoromethyl)pyrazolo[1,5-a][1,3,5]triazin-4-amine,
N-[(4,5-difluoro-1H-benzimidazol-2-yl)methyl]-2-(8-methyl-3,8-diazabicyclo[3.2.1]octan-3-yl)-8-(trifluoromethyl)pyrazolo[1,5-a][1,3,5]triazin-4-amine,
N-[(4-fluoro-1H-benzimidazol-2-yl)methyl]-2-(8-methyl-3,8-diazabicyclo[3.2.1]octan-3-yl)-8-(trifluoromethyl)pyrazolo[1,5-a][1,3,5]triazin-4-amine,
N-{[5-(3-fluorophenyl)-4H-1,2,4-triazol-3-yl]methyl}-2-(morpholin-4-yl)-8-(trifluoromethyl)pyrazolo[1,5-a][1,3,5]triazin-4-amine,
N-{[5-(3,5-difluorophenyl)-4H-1,2,4-triazol-3-yl]methyl}-2-(morpholin-4-yl)-8-(trifluoromethyl)pyrazolo[1,5-a][1,3,5]triazin-4-amine,
2-[(2S)-2,4-dimethylpiperazin-1-yl]-N-[(4-fluoro-1H-benzimidazol-2-yl)methyl]-8-(trifluoromethyl)pyrazolo[1,5-a][1,3,5]triazin-4-amine,
N-[(4,5-difluoro-1H-benzimidazol-2-yl)methyl]-8-methyl-2-(morpholin-4-yl)pyrazolo[1,5-a][1,3,5]triazin-4-amine,
1-{[4-{[(4,5-difluoro-1H-benzimidazol-2-yl)methyl]amino}-8-(trifluoromethyl)pyrazolo[1,5-a][1,3,5]triazin-2-yl](methyl)amino}-2-methylpropan-2-ol,
2-(1,4-diazabicyclo[3.2.2]nonan-4-yl)-N-[(4,5-difluoro-1H-benzimidazol-2-yl)methyl]-8-(trifluoromethyl)pyrazolo[1,5-a][1,3,5]triazin-4-amine,
2-(1,4-diazabicyclo[3.2.2]nonan-4-yl)-N-[(4-fluoro-1H-benzimidazol-2-yl)methyl]-8-(trifluoromethyl)pyrazolo[1,5-a][1,3,5]triazin-4-amine,
N-[(4,5-difluoro-1H-benzimidazol-2-yl)methyl]-2-(morpholin-4-yl)-8-(trifluoromethyl)pyrazolo [1,5-a][1,3,5]triazin-4-amine,
N-[(1H-benzimidazol-2-yl)methyl]-8-ethyl-2-(morpholin-4-yl)pyrazolo[1,5-a][1,3,5]triazin-4-amine and
N-[(4,5-difluoro-1H-benzimidazol-2-yl)methyl]-8-ethyl-2-(morpholin-4-yl)pyrazolo[1,5-a][1,3,5]triazin-4-amine.
In some embodiments, the present invention includes compounds of general formula (I) selected from:
N-(1H-benzimidazol-2-ylmethyl)-2-(morpholin-4-yl)-8-(propan-2-yl)pyrazolo[1,5-a][1,3,5]triazin-4-amine,
N-(1H-benzimidazol-2-ylmethyl)-2-(4-methylpiperazin-1-yl)-8-(propan-2-yl)pyrazolo[1,5-a][1,3,5]triazin-4-amine,
N⁴-[(1H-benzimidazol-2-yl)methyl]-N²,N²-dimethyl-8-(propan-2-yl)pyrazolo[1,5-a][1,3,5]triazine-2,4-diamine,
N-[(1H-benzimidazol-2-yl)methyl]-8-bromo-2-(morpholin-4-yl)pyrazolo[1,5-a][1,3,5]triazin-4-amine,
1-[{4-[(1H-benzimidazol-2-ylmethyl)amino]-8-(propan-2-yl)pyrazolo[1,5-a][1,3,5]triazin-2-yl}(methyl)amino]-2-methylpropan-2-ol,
N-(1H-benzimidazol-2-ylmethyl)-2-[(3S)-3-methylmorpholin-4-yl]-8-(propan-2-yl)pyrazolo[1,5-a][1,3,5]triazin-4-amine,
N-(1H-benzimidazol-2-ylmethyl)-2-[(3R)-3-methylmorpholin-4-yl]-8-(propan-2-yl)pyrazolo[1,5-a][1,3,5]triazin-4-amine,
N-(1H-benzimidazol-2-ylmethyl)-2-(1,4-oxazepan-4-yl)-8-(propan-2-yl)pyrazolo[1,5-a][1,3,5]triazin-4-amine,
N-(1H-benzimidazol-2-ylmethyl)-2-(2-oxa-6-azaspiro[3.3]hept-6-yl)-8-(propan-2-yl)pyrazolo[1,5-a][1,3,5]triazin-4-amine,
1-({4-[(1H-benzimidazol-2-ylmethyl)amino]-8-(propan-2-yl)pyrazolo[1,5-a][1,3,5]triazin-2-yl}amino)-2-methylpropan-2-ol,
N-(1H-benzimidazol-2-ylmethyl)-2-[(2S)-2-methylmorpholin-4-yl]-8-(propan-2-yl)pyrazolo[1,5-a][1,3,5]triazin-4-amine,
N-(1H-benzimidazol-2-ylmethyl)-2-(2,2-dimethylmorpholin-4-yl)-8-(propan-2-yl)pyrazolo[1,5-a][1,3,5]triazin-4-amine,
N⁴-[(1H-benzimidazol-2-yl)methyl]-N²-phenyl-8-(propan-2-yl)pyrazolo[1,5-a][1,3,5]triazine-2,4-diamine,
N⁴-[(1H-benzimidazol-2-yl)methyl]-8-(propan-2-yl)-N²-(pyridin-4-yl)pyrazolo[1,5-a][1,3,5]triazine-2,4-diamine,
N-(1H-benzimidazol-2-ylmethyl)-8-chloro-2-(morpholin-4-yl)pyrazolo[1,5-a][1,3,5]triazin-4-amine,
(3S)-1-(4-{[(1H-benzimidazol-2-yl)methyl]amino}-8-bromopyrazolo[1,5-a][1,3,5]triazin-2-yl)piperidin-3-ol,
1-(4-{[(1H-benzimidazol-2-yl)methyl]amino}-8-bromopyrazolo[1,5-a][1,3,5]triazin-2-yl)piperidin-4-ol,
N-[(1H-benzimidazol-2-yl)methyl]-8-bromo-2-(4-methylpiperazin-1-yl)pyrazolo[1,5-a][1,3,5]triazin-4-amine,
8-bromo-N-[(3H-imidazo[4,5-b]pyridin-2-yl)methyl]-2-(piperazin-1-yl)pyrazolo[1,5-a][1,3,5]triazin-4-amine formic acid salt,
8-bromo-N-[(7-methyl-3H-imidazo[4,5-b]pyridin-2-yl)methyl]-2-(morpholin-4-yl)pyrazolo[1,5-a][1,3,5]triazin-4-amine,
8-bromo-2-(morpholin-4-yl)-N-[(4,5,6,7-tetrahydro-1H-benzimidazol-2-yl)methyl]pyrazolo[1,5-a][1,3,5]triazin-4-amine,
8-bromo-N-[(3H-imidazo[4,5-c]pyridin-2-yl)methyl]-2-(morpholin-4-yl)pyrazolo[1,5-a][1,3,5]triazin-4-amine,
8-bromo-2-(morpholin-4-yl)-N-[(5-phenyl-1H-imidazol-2-yl)methyl]pyrazolo[1,5-a][1,3,5]triazin-4-amine,
8-bromo-2-(morpholin-4-yl)-N-[(5-phenyl-4H-1,2,4-triazol-3-yl)methyl]pyrazolo[1,5-a][1,3,5]triazin-4-amine,
8-bromo-N-[(4,5-difluoro-1H-benzimidazol-2-yl)methyl]-2-(morpholin-4-yl)pyrazolo[1,5-a][1,3,5]triazin-4-amine,
8-bromo-N²-ethyl-N⁴-{[4-(4-methylphenyl)-1H-imidazol-2-yl]methyl}pyrazolo[1,5-a][1,3,5]triazine-2,4-diamine,
8-bromo-N-[(5-methyl-4-phenyl-1H-imidazol-2-yl)methyl]-2-(morpholin-4-yl)pyrazolo[1,5-a][1,3,5]triazin-4-amine,
N-[(7-methyl-3H-imidazo[4,5-b]pyridin-2-yl)methyl]-2-(morpholin-4-yl)-8-(propan-2-yl)pyrazolo[1,5-a][1,3,5]triazin-4-amine,
8-bromo-N-{[5-(4-fluorophenyl)-4H-1,2,4-triazol-3-yl]methyl}-2-(morpholin-4-yl)pyrazolo[1,5-a][1,3,5]triazin-4-amine,
2-(morpholin-4-yl)-N-[(5-phenyl-1H-imidazol-2-yl)methyl]-8-(propan-2-yl)pyrazolo[1,5-a][1,3,5]triazin-4-amine,
2-(4-methylpiperazin-1-yl)-N-[(5-phenyl-1H-imidazol-2-yl)methyl]-8-(propan-2-yl)pyrazolo[1,5-a][1,3,5]triazin-4-amine,
N-[(7-methyl-3H-imidazo[4,5-b]pyridin-2-yl)methyl]-2-(4-methylpiperazin-1-yl)-8-(propan-2-yl)pyrazolo[1,5-a][1,3,5]triazin-4-amine,
2-(morpholin-4-yl)-N-[(5-phenyl-4H-1,2,4-triazol-3-yl)methyl]-8-(propan-2-yl)pyrazolo[1,5-a][1,3,5]triazin-4-amine,
N²-ethyl-N⁴-[(5-phenyl-1H-imidazol-2-yl)methyl]-8-(propan-2-yl)pyrazolo[1,5-a][1,3,5]triazine-2,4-diamine,
2-methyl-1-{methyl[4-{[(5-phenyl-1H-imidazol-2-yl)methyl]amino}-8-(propan-2-yl)pyrazolo[1,5-a][1,3,5]triazin-2-yl]amino}propan-2-ol,
N-[(5-phenyl-1H-imidazol-2-yl)methyl]-2-(piperidin-1-yl)-8-(propan-2-yl)pyrazolo[1,5-a][1,3,5]triazin-4-amine,
2-(4-methylpiperazin-1-yl)-N-[(5-phenyl-4H-1,2,4-triazol-3-yl)methyl]-8-(propan-2-yl)pyrazolo[1,5-a][1,3,5]triazin-4-amine,
N²-ethyl-N⁴-[(5-phenyl-4H-1,2,4-triazol-3-yl)methyl]-8-(propan-2-yl)pyrazolo[1,5-a][1,3,5]triazine-2,4-diamine,
2-methyl-1-{methyl[4-{[(5-phenyl-4H-1,2,4-triazol-3-yl)methyl]amino}-8-(propan-2-yl)pyrazolo[1,5-a][1,3,5]triazin-2-yl]amino}propan-2-ol,
8-bromo-N-{[5-(3-methylphenyl)-4H-1,2,4-triazol-3-yl]methyl}-2-(morpholin-4-yl)pyrazolo[1,5-a][1,3,5]triazin-4-amine,
8-bromo-N-{[5-(2,4-difluorophenyl)-4H-1,2,4-triazol-3-yl]methyl}-2-(morpholin-4-yl)pyrazolo[1,5-a][1,3,5]triazin-4-amine,
3-[2-({[8-bromo-2-(morpholin-4-yl)pyrazolo[1,5-a][1,3,5]triazin-4-yl]amino}methyl)-1H-imidazol-4-yl]benzonitrile,
8-bromo-N-{[5-(3-fluorophenyl)-4H-1,2,4-triazol-3-yl]methyl}-2-(morpholin-4-yl)pyrazolo[1,5-a][1,3,5]triazin-4-amine,
8-bromo-N-{[5-(4-chlorophenyl)-4H-1,2,4-triazol-3-yl]methyl}-2-(morpholin-4-yl)pyrazolo[1,5-a][1,3,5]triazin-4-amine,
N-[(1H-benzimidazol-2-yl)methyl]-8-bromo-2-(piperidin-1-yl)pyrazolo[1,5-a][1,3,5]triazin-4-amine,
N-[(1H-benzimidazol-2-yl)methyl]-8-bromo-2-(pyrrolidin-1-yl)pyrazolo[1,5-a][1,3,5]triazin-4-amine,
N-[(1H-benzimidazol-2-yl)methyl]-8-bromo-2-(2-oxa-6-azaspiro[3.3]heptan-6-yl)pyrazolo[1,5-a][1,3,5]triazin-4-amine,
8-bromo-N-[(5-phenyl-4H-1,2,4-triazol-3-yl)methyl]-2-(piperidin-1-yl)pyrazolo[1,5-a][1,3,5]triazin-4-amine,
8-bromo-N-{[5-(3-methoxyphenyl)-4H-1,2,4-triazol-3-yl]methyl}-2-(morpholin-4-yl)pyrazolo[1,5-a][1,3,5]triazin-4-amine,
8-bromo-N-{[5-(3-chlorophenyl)-4H-1,2,4-triazol-3-yl]methyl}-2-(morpholin-4-yl)pyrazolo[1,5-a][1,3,5]triazin-4-amine,
8-bromo-2-(morpholin-4-yl)-N-({5-[3-(trifluoromethyl)phenyl]-4H-1,2,4-triazol-3-yl}methyl)pyrazolo[1,5-a][1,3,5]triazin-4-amine,
8-bromo-2-(morpholin-4-yl)-N-{[5-(pyridin-3-yl)-4H-1,2,4-triazol-3-yl]methyl}pyrazolo[1,5-a][1,3,5]triazin-4-amine,
8-bromo-2-(morpholin-4-yl)-N-{[5-(pyridin-2-yl)-4H-1,2,4-triazol-3-yl]methyl}pyrazolo[1,5-a][1,3,5]triazin-4-amine,
3-[5-({[8-bromo-2-(morpholin-4-yl)pyrazolo[1,5-a][1,3,5]triazin-4-yl]amino}methyl)-4H-1,2,4-triazol-3-yl]benzonitrile,
N-[(1H-benzimidazol-2-yl)methyl]-8-bromo-2-[(3R)-3-methylmorpholin-4-yl]pyrazolo[1,5-a][1,3,5]triazin-4-amine,
N-[(1H-benzimidazol-2-yl)methyl]-8-bromo-2-[(3S)-3-methylmorpholin-4-yl]pyrazolo[1,5-a][1,3,5]triazin-4-amine,
N-[(1H-benzimidazol-2-yl)methyl]-8-bromo-2-(2,2-dimethylmorpholin-4-yl)pyrazolo[1,5-a][1,3,5]triazin-4-amine,
[(2S)-1-(4-{[(1H-benzimidazol-2-yl)methyl]amino}-8-bromopyrazolo[1,5-a][1,3,5]triazin-2-yl)piperidin-2-yl]methanol,
[(2R)-1-(4-{[(1H-benzimidazol-2-yl)methyl]amino}-8-bromopyrazolo[1,5-a][1,3,5]triazin-2-yl)piperidin-2-yl]methanol,
8-bromo-N-{[5-(2-methoxyphenyl)-4H-1,2,4-triazol-3-yl]methyl}-2-(morpholin-4-yl)pyrazolo[1,5-a][1,3,5]triazin-4-amine,
N-[(1H-benzimidazol-2-yl)methyl]-8-bromo-2-(4-methoxypiperidin-1-yl)pyrazolo[1,5-a][1,3,5]triazin-4-amine,
methyl 1-(4-{[(1H-benzimidazol-2-yl)methyl]amino}-8-bromopyrazolo[1,5-a][1,3,5]triazin-2-yl)piperidine-4-carboxylate,
8-bromo-N-[(5-phenyl-4H-1,2,4-triazol-3-yl)methyl]-2-(pyrrolidin-1-yl)pyrazolo[1,5-a][1,3,5]triazin-4-amine,
8-bromo-N-{[5-(4-fluorophenyl)-1H-imidazol-2-yl]methyl}-2-(morpholin-4-yl)pyrazolo[1,5-a][1,3,5]triazin-4-amine,
8-bromo-2-(2-oxa-6-azaspiro[3.3]heptan-6-yl)-N-[(5-phenyl-4H-1,2,4-triazol-3-yl)methyl]pyrazolo[1,5-a][1,3,5]triazin-4-amine,
[(3R)-4-(4-{[(1H-benzimidazol-2-yl)methyl]amino}-8-bromopyrazolo[1,5-a][1,3,5]triazin-2-yl)morpholin-3-yl]methanol,
8-bromo-2-(2,2-dimethylmorpholin-4-yl)-N-[(5-phenyl-4H-1,2,4-triazol-3-yl)methyl]pyrazolo[1,5-a][1,3,5]triazin-4-amine,
8-bromo-N-{[5-(2-chlorophenyl)-4H-1,2,4-triazol-3-yl]methyl}-2-(morpholin-4-yl)pyrazolo[1,5-a][1,3,5]triazin-4-amine,
[(2S)-1-(8-bromo-4-{[(5-phenyl-4H-1,2,4-triazol-3-yl)methyl]amino}pyrazolo[1,5-a][1,3,5]triazin-2-yl)piperidin-2-yl]methanol,
[(2R)-1-(8-bromo-4-{[(5-phenyl-4H-1,2,4-triazol-3-yl)methyl]amino}pyrazolo[1,5-a][1,3,5]triazin-2-yl)piperidin-2-yl]methanol,
8-bromo-N-{[5-(4-chlorophenyl)-1H-imidazol-2-yl]methyl}-2-(morpholin-4-yl)pyrazolo[1,5-a][1,3,5]triazin-4-amine,
N-[(1H-benzimidazol-2-yl)methyl]-8-bromo-2-[(2R,6S)-2,6-dimethylmorpholin-4-yl]pyrazolo[1,5-a][1,3,5]triazin-4-amine,
8-bromo-2-(morpholin-4-yl)-N-({5-[2-(trifluoromethoxy)phenyl]-4H-1,2,4-triazol-3-yl}methyl)pyrazolo[1,5-a][1,3,5]triazin-4-amine,
8-bromo-N-{[5-(2-fluorophenyl)-4H-1,2,4-triazol-3-yl]methyl}-2-(morpholin-4-yl)pyrazolo[1,5-a][1,3,5]triazin-4-amine,
8-bromo-2-(4-methoxypiperidin-1-yl)-N-[(5-phenyl-4H-1,2,4-triazol-3-yl)methyl]pyrazolo[1,5-a][1,3,5]triazin-4-amine,
methyl 1-(8-bromo-4-{[(5-phenyl-4H-1,2,4-triazol-3-yl)methyl]amino}pyrazolo[1,5-a][1,3,5]triazin-2-yl)piperidine-4-carboxylate,
8-bromo-2-[(2R,6S)-2,6-dimethylmorpholin-4-yl]-N-[(5-phenyl-4H-1,2,4-triazol-3-yl)methyl]pyrazolo[1,5-a][1,3,5]triazin-4-amine,
8-bromo-N-{[5-(3-fluorophenyl)-1H-imidazol-2-yl]methyl}-2-(morpholin-4-yl)pyrazolo[1,5-a][1,3,5]triazin-4-amine,
8-bromo-N-{[5-(3-methoxyphenyl)-1H-imidazol-2-yl]methyl}-2-(morpholin-4-yl)pyrazolo[1,5-a][1,3,5]triazin-4-amine,
8-bromo-N-{[5-(3-chlorophenyl)-1H-imidazol-2-yl]methyl}-2-(morpholin-4-yl)pyrazolo[1,5-a][1,3,5]triazin-4-amine,
8-bromo-2-(morpholin-4-yl)-N-({5-[3-(trifluoromethoxy)phenyl]-1H-imidazol-2-yl}methyl)pyrazolo[1,5-a][1,3,5]triazin-4-amine,
8-bromo-2-(morpholin-4-yl)-N-({5-[3-(trifluoromethyl)phenyl]-1H-imidazol-2-yl}methyl)pyrazolo[1,5-a][1,3,5]triazin-4-amine,
8-bromo-2-(morpholin-4-yl)-N-{[5-(pyridin-3-yl)-1H-imidazol-2-yl]methyl}pyrazolo[1,5-a][1,3,5]triazin-4-amine,
8-bromo-N-{[5-(2-methylphenyl)-1H-imidazol-2-yl]methyl}-2-(morpholin-4-yl)pyrazolo[1,5-a][1,3,5]triazin-4-amine,
8-bromo-N-{[5-(2-fluorophenyl)-1H-imidazol-2-yl]methyl}-2-(morpholin-4-yl)pyrazolo[1,5-a][1,3,5]triazin-4-amine,
8-bromo-N-{[5-(2-methoxyphenyl)-1H-imidazol-2-yl]methyl}-2-(morpholin-4-yl)pyrazolo[1,5-a][1,3,5]triazin-4-amine,
8-bromo-2-(morpholin-4-yl)-N-({5-[2-(trifluoromethyl)phenyl]-1H-imidazol-2-yl}methyl)pyrazolo[1,5-a][1,3,5]triazin-4-amine,
8-bromo-2-(morpholin-4-yl)-N-({5-[2-(trifluoromethoxy)phenyl]-1H-imidazol-2-yl}methyl)pyrazolo[1,5-a][1,3,5]triazin-4-amine,
8-bromo-2-(morpholin-4-yl)-N-{[5-(pyridin-2-yl)-1H-imidazol-2-yl]methyl}pyrazolo[1,5-a][1,3,5]triazin-4-amine,
N-[(1H-benzimidazol-2-yl)methyl]-8-(3-fluorophenyl)-2-(morpholin-4-yl)pyrazolo[1,5-a][1,3,5]triazin-4-amine,
N-[(1H-benzimidazol-2-yl)methyl]-8-cyclopropyl-2-(morpholin-4-yl)pyrazolo[1,5-a][1,3,5]triazin-4-amine,
8-cyclopropyl-2-(morpholin-4-yl)-N-[(5-phenyl-4H-1,2,4-triazol-3-yl)methyl]pyrazolo[1,5-a][1,3,5]triazin-4-amine,
8-cyclopropyl-2-(2,2-dimethylmorpholin-4-yl)-N-[(5-phenyl-4H-1,2,4-triazol-3-yl)methyl]pyrazolo[1,5-a][1,3,5]triazin-4-amine,
N-[(1H-benzimidazol-2-yl)methyl]-8-(2-fluorophenyl)-2-(morpholin-4-yl)pyrazolo[1,5-a][1,3,5]triazin-4-amine,
8-bromo-N-{[5-(2-chlorophenyl)-1H-imidazol-2-yl]methyl}-2-(morpholin-4-yl)pyrazolo[1,5-a][1,3,5]triazin-4-amine,
8-bromo-N-[(4-fluoro-1H-benzimidazol-2-yl)methyl]-2-(morpholin-4-yl)pyrazolo[1,5-a][1,3,5]triazin-4-amine,
8-bromo-N-{[5-(3-fluoro-4-methylphenyl)-4H-1,2,4-triazol-3-yl]methyl}-2-(morpholin-4-yl)pyrazolo[1,5-a][1,3,5]triazin-4-amine,
8-bromo-N-{[5-(3,4-difluorophenyl)-4H-1,2,4-triazol-3-yl]methyl}-2-(morpholin-4-yl)pyrazolo[1,5-a][1,3,5]triazin-4-amine,
8-bromo-2-(morpholin-4-yl)-N-{[5-(3,4,5-trifluorophenyl)-4H-1,2,4-triazol-3-yl]methyl}pyrazolo[1,5-a][1,3,5]triazin-4-amine,
8-bromo-N-[(5-fluoro-1H-benzimidazol-2-yl)methyl]-2-(morpholin-4-yl)pyrazolo[1,5-a][1,3,5]triazin-4-amine,
N-[(1H-benzimidazol-2-yl)methyl]-8-bromo-2-[(1R,4R)-2-oxa-5-azabicyclo[2.2.1]heptan-5-yl]pyrazolo[1,5-a][1,3,5]triazin-4-amine,
N-[(1H-benzimidazol-2-yl)methyl]-8-bromo-2-[(1S,4S)-2-oxa-5-azabicyclo[2.2.1]heptan-5-yl]pyrazolo[1,5-a][1,3,5]triazin-4-amine,
N-[(1H-benzimidazol-2-yl)methyl]-2-(morpholin-4-yl)-8-phenylpyrazolo[1,5-a][1,3,5]triazin-4-amine,
8-bromo-N-{[5-(3,5-difluorophenyl)-4H-1,2,4-triazol-3-yl]methyl}-2-(morpholin-4-yl)pyrazolo[1,5-a][1,3,5]triazin-4-amine,
N-[(1H-benzimidazol-2-yl)methyl]-8-(4-fluorophenyl)-2-(morpholin-4-yl)pyrazolo[1,5-a][1,3,5]triazin-4-amine,
N-[(1H-benzimidazol-2-yl)methyl]-8-(3-chlorophenyl)-2-(morpholin-4-yl)pyrazolo[1,5-a][1,3,5]triazin-4-amine,
N-[(1H-benzimidazol-2-yl)methyl]-2-(morpholin-4-yl)-8-(pyridin-3-yl)pyrazolo[1,5-a][1,3,5]triazin-4-amine,
8-bromo-2-[(2R,6S)-2,6-dimethylmorpholin-4-yl]-N-[(4-fluoro-1H-benzimidazol-2-yl)methyl]pyrazolo[1,5-a][1,3,5]triazin-4-amine,
8-bromo-2-[(2R,6S)-2,6-dimethylmorpholin-4-yl]-N-[(5-fluoro-1H-benzimidazol-2-yl)methyl]pyrazolo[1,5-a][1,3,5]triazin-4-amine and
N-[(1H-benzimidazol-2-yl)methyl]-2-(morpholin-4-yl)-8-(pyridin-4-yl)pyrazolo[1,5-a][1,3,5]triazin-4-amine hydrogen chloride (1:1).

As used herein and in the context of the present invention, the term “IC50 CDK12 hATP” refers to the IC₅₀ values obtained according to the assay described in section 2.2 of the Experimental Section herein below, i.e. the IC₅₀ values for the inhibition of CDK12 at high ATP.

As used herein and in the context of the present invention, the term “DC50 CDK12” refers to the DC₅₀ values obtained according to the assay described in section 7 of the Experimental Section herein below, i.e. the DC₅₀ values for the degradation of CDK12. In some embodiments, the present invention includes compounds of general formula (I), supra, which show a ratio (IC50 CDK12 hATP)/(DC50 CDK12) which is equal or greater than 5.

In some embodiments, the present invention includes compounds of general formula (I), supra, which show a ratio (IC50 CDK12 hATP)/(DC50 CDK12) which is greater than 5.

In some embodiments, the present invention includes compounds of general formula (I), supra, which show a ratio (IC50 CDK12 hATP)/(DC50 CDK12) which is equal or greater than 10.

In some embodiments, the present invention includes compounds of general formula (I), supra, which show a ratio (IC50 CDK12 hATP)/(DC50 CDK12) which is greater than 10.

In some embodiments, the present invention includes compounds of general formula (I), supra, which show a ratio (IC50 CDK12 hATP)/(DC50 CDK12) which is equal or greater than 20.

In some embodiments, the present invention includes compounds of general formula (I), supra, which show a ratio (IC50 CDK12 hATP)/(DC50 CDK12) which is greater than 20.

In some embodiments, the present invention includes compounds of general formula (I), supra, which show a ratio (IC50 CDK12 hATP)/(DC50 CDK12) which is equal or greater than 30.

In some embodiments, the present invention includes compounds of general formula (I), supra, which show a ratio (IC50 CDK12 hATP)/(DC50 CDK12) which is greater than 30.

In some embodiments, the present invention includes compounds of general formula (I), supra, which show a ratio (IC50 CDK12 hATP)/(DC50 CDK12) which is equal or greater than 50.

In some embodiments, the present invention includes compounds of general formula (I), supra, which show a ratio (IC50 CDK12 hATP)/(DC50 CDK12) which is greater than 50.

In some embodiments, the present invention includes compounds of general formula (I), supra, which show a ratio (IC50 CDK12 hATP)/(DC50 CDK12) which is greater than 5 and a (DC50 CDK12) value which is lower than 200 nM.

In some embodiments, the present invention includes compounds of general formula (I), supra, which show a ratio (IC50 CDK12 hATP)/(DC50 CDK12) which is greater than 5 and a (DC50 CDK12) value which is lower than 20 nM.

In some embodiments, the present invention includes compounds of general formula (I), supra, which show a ratio (IC50 CDK12 hATP)/(DC50 CDK12) which is greater than 5 and a (DC50 CDK12) value which is lower than 2 nM.

In some embodiments, the present invention includes compounds of general formula (I), supra, which show a ratio (IC50 CDK12 hATP)/(DC50 CDK12) which is greater than 10 and a (DC50 CDK12) value which is lower than 200 nM.

In some embodiments, the present invention includes compounds of general formula (I), supra, which show a ratio (IC50 CDK12 hATP)/(DC50 CDK12) which is greater than 10 and a (DC50 CDK12) value which is lower than 20 nM.

In some embodiments, the present invention includes compounds of general formula (I), supra, which show a ratio (IC50 CDK12 hATP)/(DC50 CDK12) which is greater than 10 and a (DC50 CDK12) value which is lower than 2 nM.

In some embodiments, the present invention includes compounds of general formula (I), supra, which show a ratio (IC50 CDK12 hATP)/(DC50 CDK12) which is equal or greater than 20 and a (DC50 CDK12) value which is equal or lower than 200 nM.

In some embodiments, the present invention includes compounds of general formula (I), supra, which show a ratio (IC50 CDK12 hATP)/(DC50 CDK12) which is greater than 20 and a (DC50 CDK12) value which is lower than 200 nM.

In some embodiments, the present invention includes compounds of general formula (I), supra, which show a ratio (IC50 CDK12 hATP)/(DC50 CDK12) which is greater than 20 and a (DC50 CDK12) value which is lower than 20 nM.

In some embodiments, the present invention includes compounds of general formula (I), supra, which show a ratio (IC50 CDK12 hATP)/(DC50 CDK12) which is greater than 20 and a (DC50 CDK12) value which is lower than 2 nM.

### Further Embodiments of the First Aspect of the Present Invention

In some embodiments, the present invention provides compounds of formula (I), supra, in which R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₄-alkyl group, a C₁-C₃-haloalkyl group, a C₃-C₆-cycloalkyl group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a C₁-C₆-alkyl group, a C₁-C₆-alkoxy group, a C₁-C₆-haloalkyl group,
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₄-alkyl group, a C₁-C₃-haloalkyl group, a C₃-C₆-cycloalkyl group, a cyano group, a phenyl group, a heterocycloalkyl group and a heteroaryl group,
wherein said C₁-C₄-alkyl, C₃-C₆-cycloalkyl, phenyl, heterocycloalkyl or heteroaryl group is each optionally substituted with one or more substituents independently selected from a halogen atom and a C₁-C₆-alkyl group;

In some embodiments, the present invention provides compounds of formula (I), supra, in which R¹ is selected from a halogen atom, a C₁-C₆-alkyl group, a C₁-C₆-haloalkyl group, a C₃-C₈-cycloalkyl group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₄-alkyl group, a C₁-C₄-haloalkyl group, a C₃-C₄-cycloalkyl group, a a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₄-alkyl group, a C₁-C₃-haloalkyl group, a C₃-C₆-cycloalkyl group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group,
   or a tautomer, or an N-oxide, or a salt thereof, or a salt of a tautomer, or a salt of an N-oxide, or a mixture of same.

In some embodiments, the present invention provides compounds of formula (I), supra, in which R¹ is selected from a halogen atom, a C₁-C₃-alkyl group, a C₁-C₃-haloalkyl group, a C₃-C₆-cycloalkyl group and a C₃-C₆-halocycloalkyl group.

In some embodiments, the present invention provides compounds of formula (I), supra, in which R¹ is selected from a halogen atom, a cyano group, a C₁-C₃-alkyl group, a C₁-C₃-haloalkyl group, a C₃-C₆-cycloalkyl group and a C₃-C₆-halocycloalkyl group.

In some embodiments, the present invention provides compounds of formula (I), supra, in which R¹ is selected from a halogen atom, a cyano group, a C₁-C₃-alkyl group, a C₁-C₃-haloalkyl group and a C₃-C₆-cycloalkyl group.

In some embodiments, the present invention provides compounds of formula (I), supra, in which R¹ is selected from a halogen atom, a cyano group, a C₁-C₃-alkyl group and a C₃-C₆-cycloalkyl group.

In some embodiments, the present invention provides compounds of formula (I), supra, in which R¹ is selected from a halogen atom, a cyano group, a C₁-C₃-alkyl group and a C₁-C₃-haloalkyl group.

In some embodiments, the present invention provides compounds of formula (I), supra, in which R¹ is selected from a halogen atom, a cyano group, a C₁-C₃-alkyl group and a trifluoromethyl group.

In some embodiments, the present invention provides compounds of formula (I), supra, in which R¹ is selected from a halogen atom, a cyano group, a C₁-C₃-alkyl group and a C₃-C₆-halocycloalkyl group.

In some embodiments, the present invention provides compounds of formula (I), supra, in which R¹ is selected from a halogen atom, a cyano group and a C₃-C₆-halocycloalkyl group.

In some embodiments, the present invention provides compounds of formula (I), supra, in which R¹ is selected from a halogen atom and a cyano group.

In some embodiments, the present invention provides compounds of formula (I), supra, in which R¹ is selected from a C₁-C₆-alkyl group, a C₁-C₆-haloalkyl group, a C₃-C₈-cycloalkyl group, a C₃-C₈-halocycloalkyl group and a (C₃-C₈-cycloalkyl)-(C₁-C₆-alkyl)- group.

In some embodiments, the present invention provides compounds of formula (I), supra, in which R¹ is selected from a C₁-C₆-alkyl group, a C₁-C₆-haloalkyl group, a C₃-C₈-cycloalkyl group, a (C₃-C₈-cycloalkyl)-(C₁-C₆-alkyl)- group.

In some embodiments, the present invention provides compounds of formula (I), supra, in which R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a heterocycloalkyl group and a —NR^{a}R^{b} group,
wherein said heterocycloalkyl group is connected to the rest of the molecule via a carbon atom of said heterocycloalkyl group,
wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
   wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group and a R⁵R⁶N— group
wherein at least one of R^{a} or R^{b} is not a hydrogen atom;
or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
   said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—
   and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo
      or a tautomer, or an N-oxide, or a salt thereof, or a salt of a tautomer, or a salt of an N-oxide, or a mixture of same.

In some embodiments, the present invention provides compounds of formula (I), supra, in which R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a (C₃-C₆-cycloalkyl)-(C₁-C₂-alkyl)-O— group, a (C₃-C₆-hydroxyalkyl)-(C₁-C₂-alkyl)-O— group, a (C₃-C₆-alkoxyalkyl)-(C₁-C₂-alkyl)-O— group, a ((CH₃)₂N)—(C₁-C₂-alkyl)-O— group, a heterocycloalkyl group, a (heterocycloalkyl)-O— group and a —NR^{a}R^{b} group,
wherein said heterocycloalkyl group is connected to the rest of the molecule via a carbon atom of said heterocycloalkyl group,
wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
   wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group and a R⁵R⁶N— group
wherein at least one of R^{a} or R^{b} is not a hydrogen atom;
or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
   said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—
   and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo
      or a tautomer, or an N-oxide, or a salt thereof, or a salt of a tautomer, or a salt of an N-oxide, or a mixture of same.

In some embodiments, the present invention provides compounds of formula (I), supra, in which R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a heterocycloalkyl group and a —NR^{a}R^{b} group,
wherein said heterocycloalkyl group is connected to the rest of the molecule via a carbon atom of said heterocycloalkyl group,
wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
   wherein said C₃-C₈-cycloalkyl, phenyl, heteroaryl or heterocycloalkyl group is each optionally substituted with one or more substituents independently selected from a halogen atom, a hydroxy group, a cyano group, a C₁-alkyl group, a C₁-hydroxyalkyl group, a C₁-haloalkyl group, a C₁-alkoxy group, a C₁-haloalkoxy group and a R⁵R⁶N— group
wherein at least one of R^{a} or R^{b} is not a hydrogen atom;
or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
   said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—
   and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo
      or a tautomer, or an N-oxide, or a salt thereof, or a salt of a tautomer, or a salt of an N-oxide, or a mixture of same.

In some embodiments, the present invention provides compounds of formula (I), supra, in which R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a heterocycloalkyl group and a —NR^{a}R^{b} group,
wherein said heterocycloalkyl group is connected to the rest of the molecule via a carbon atom of said heterocycloalkyl group,
wherein R^{a} and R^{b} are each independently selected from a hydrogen atom, a C₁-C₆-alkyl group, a C₁-C₆-hydroxyalkyl group, a heteroaryl group and a phenyl group,
   wherein said phenyl, heteroaryl or heterocycloalkyl group is each optionally substituted with one or more substituents independently selected from a halogen atom, a cyano group, a C₁-alkyl group, a C₁-hydroxyalkyl group, a C₁-haloalkyl group, a C₁-alkoxy group, a C₁-haloalkoxy group and a R⁵R⁶N— group
   wherein at least one of R^{a} or R^{b} is not a hydrogen atom;
or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
   said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—
   and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo
      or a tautomer, or an N-oxide, or a salt thereof, or a salt of a tautomer, or a salt of an N-oxide, or a mixture of same.

In some embodiments, the present invention provides compounds of formula (I), supra, in which R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a (C₃-C₆-cycloalkyl)-(C₁-C₂-alkyl)-O— group, a (C₃-C₆-hydroxyalkyl)-(C₁-C₂-alkyl)-O— group, a (C₃-C₆-alkoxyalkyl)-(C₁-C₂-alkyl)-O— group, a ((CH₃)₂N)—(C₁-C₂-alkyl)-O— group, a heterocycloalkyl group, a (heterocycloalkyl)-O— group and a —NR^{a}R^{b} group,
wherein said heterocycloalkyl group is connected to the rest of the molecule via a carbon atom of said heterocycloalkyl group,
wherein R^{a} and R^{b} are each independently selected from a hydrogen atom, a C₁-C₆-alkyl group, a C₁-C₆-hydroxyalkyl group, a heteroaryl group and a phenyl group,
   wherein said phenyl, heteroaryl or heterocycloalkyl group is each optionally substituted with one or more substituents independently selected from a halogen atom, a cyano group, a C₁-alkyl group, a C₁-hydroxyalkyl group, a C₁-haloalkyl group, a C₁-alkoxy group, a C₁-haloalkoxy group and a R⁵R⁶N— group
   wherein at least one of R^{a} or R^{b} is not a hydrogen atom;
or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
   said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—
   and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo or a tautomer, or an N-oxide, or a salt thereof, or a salt of a tautomer, or a salt of an N-oxide, or a mixture of same.

In some embodiments, the present invention provides compounds of formula (I), supra, in which R² is selected from a C₁-C₆-alkoxy group, a C₁-C₆-haloalkoxy group, a C₃-C₈-cycloalkoxy group, a (C₃-C₆-cycloalkyl)-(C₁-C₂-alkyl)-O— group, a (C₃-C₆-hydroxyalkyl)-(C₁-C₂-alkyl)-O— group, a (C₃-C₆-alkoxyalkyl)-(C₁-C₂-alkyl)-O— group, a ((CH₃)₂N)—(C₁-C₂-alkyl)-O— group and a (heterocycloalkyl)-O— group,
wherein said heterocycloalkyl group is connected to the rest of the molecule via a carbon atom of said heterocycloalkyl group,
   or a tautomer, or an N-oxide, or a salt thereof, or a salt of a tautomer, or a salt of an N-oxide, or a mixture of same.

In some embodiments, the present invention provides compounds of formula (I), supra, in which R² is a —NR^{a}R^{b} group,
wherein R^{a} and R^{b} are each independently selected from a hydrogen atom, a C₁-C₆-alkyl group, a C₁-C₆-hydroxyalkyl group, a heteroaryl group and a phenyl group,
   wherein said phenyl, heteroaryl or heterocycloalkyl group is each optionally substituted with one or more substituents independently selected from a halogen atom, a cyano group, a C₁-alkyl group, a C₁-hydroxyalkyl group, a C₁-haloalkyl group, a C₁-alkoxy group, a C₁-haloalkoxy group and a R⁵R⁶N— group
   wherein at least one of R^{a} or R^{b} is not a hydrogen atom;
or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
   said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—
   and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo
      or a tautomer, or an N-oxide, or a salt thereof, or a salt of a tautomer, or a salt of an N-oxide, or a mixture of same.

In some embodiments, the present invention provides compounds of formula (I), supra, in which R² is a —NR^{a}R^{b} group,
wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
   said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—
   and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo
      or a tautomer, or an N-oxide, or a salt thereof, or a salt of a tautomer, or a salt of an N-oxide, or a mixture of same.

In some embodiments, the present invention provides compounds of formula (I), supra, in which R² is a —NR^{a}R^{b} group,
wherein R^{a} and R^{b} together with the nitrogen atom to which they are attached form a 4- to 9-membered nitrogen-containing or 5- to 11-membered nitrogen-containing bicyclic heterocycloalkyl group, a 7- to 9-membered nitrogen-containing bridged compound or a 7- to 12-membered nitrogen-containing spiro compound,
   said 4- to 9-membered nitrogen-containing or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
   and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo
      or a tautomer, or an N-oxide, or a salt thereof, or a salt of a tautomer, or a salt of an N-oxide, or a mixture of same.

In some embodiments, the present invention provides compounds of formula (I), supra, in which R² is a —NR^{a}R^{b} group,
wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
   said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound containing one, two or three further heteroatoms independently selected from, oxygen and sulfur or one group selected from —S(═O)—, —S(═O)₂— and —S(═O)(═NH)—
   and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo
      or a tautomer, or an N-oxide, or a salt thereof, or a salt of a tautomer, or a salt of an N-oxide, or a mixture of same.

In some embodiments, the present invention provides compounds of formula (I), supra, in which R² is a —NR^{a}R^{b} group,
wherein R^{a} and R^{b} together with the nitrogen atom to which they are attached form a 4- to 9-membered nitrogen-containing monocyclic or 5- to 11-membered nitrogen-containing bicyclic heterocycloalkyl group,
   said 4- to 9-membered nitrogen-containing monocyclic or 5- to 11-membered nitrogen-containing bicyclic heterocycloalkyl group containing one, two or three further heteroatoms independently selected from, oxygen and sulfur or one group selected from —S(═O)—, —S(═O)₂— and —S(═O)(═NH)—
   and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo
      or a tautomer, or an N-oxide, or a salt thereof, or a salt of a tautomer, or a salt of an N-oxide, or a mixture of same.

In some embodiments, the present invention provides compounds of formula (I), supra, in which R² is a —NR^{a}R^{b} group,
wherein R^{a} and R^{b} together with the nitrogen atom to which they are attached form a morpholine ring,
   said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo
      or a tautomer, or an N-oxide, or a salt thereof, or a salt of a tautomer, or a salt of an N-oxide, or a mixture of same.

In some embodiments, the present invention provides compounds of formula (I), supra, in which
R¹ is selected from a halogen atom and a cyano group;
R² is a —NR^{a}R^{b} group,
   wherein R^{a} and R^{b} are each independently selected from a hydrogen atom, a C₁-C₃-alkyl group, a C₃-C₆-cycloalkyl group, a C₁-C₃-haloalkyl group, a (C₃-C₆-cycloalkyl)-(C₁-C₃-alkyl)- group, a heterocycloalkyl group, a heteroaryl group and a phenyl group,
   wherein said C₃-C₆-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁a C₁-C₃-haloalkyl group, a C₁-C₃-alkoxy group, a C₁-C₃-haloalkoxy group and a R⁵R⁶N— group
   wherein at least one of R^{a} or R^{b} is not a hydrogen atom;
   or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—
      wherein said 4- to 9-membered nitrogen-containing monocyclic, 5- to 11-membered nitrogen-containing bicyclic heterocycloalkyl group, 7- to 9-membered nitrogen-containing bridged compound or 7- to 12-membered nitrogen-containing spiro compound each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo,
      wherein when R^{a} and R^{b} together with the nitrogen atom to which they are attached form a pyrrolidine ring, a piperidine ring or a piperazine ring, said pyrrolidine, piperidine or piperazine ring is not substituted in any of its carbon atoms;
         or a tautomer, or an N-oxide, or a salt thereof, or a salt of a tautomer, or a salt of an N-oxide, or a mixture of same.

In some embodiments, the present invention provides compounds of formula (I), supra, in which
R¹ is selected from a halogen atom and a cyano group;
R² is a —NR^{a}R^{b} group,
   wherein R^{a} and R^{b} are each independently selected from a hydrogen atom, a C₁-C₃-alkyl group, a C₃-C₆-cycloalkyl group, a C₁-C₃-haloalkyl group, a (C₃-C₆-cycloalkyl)-(C₁-C₃-alkyl)- group, a heterocycloalkyl group, a heteroaryl group and a phenyl group,
   wherein said C₃-C₆-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁a C₁-C₃-haloalkyl group, a C₁-C₃-alkoxy group, a C₁-C₃-haloalkoxy group and a R⁵R⁶N— group
   wherein at least one of R^{a} or R^{b} is not a hydrogen atom;
   or R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group, a 7- to 9-membered nitrogen-containing bridged compound or a 7- to 12-membered nitrogen-containing spiro compound,
      said or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—
      wherein said azetidine ring, morpholine ring, 5- to 11-membered nitrogen-containing bicyclic heterocycloalkyl group, 7- to 9-membered nitrogen-containing bridged compound or 7- to 12-membered nitrogen-containing spiro compound each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
         or a tautomer, or an N-oxide, or a salt thereof, or a salt of a tautomer, or a salt of an N-oxide, or a mixture of same.

In some embodiments, the present invention provides compounds of formula (I), supra, in which
R¹ is selected from a halogen atom and a cyano group;
R² is a —NR^{a}R^{b} group,
   wherein R^{a} and R^{b} are each independently selected from a hydrogen atom, a C₁-C₃-alkyl group, a C₃-C₆-cycloalkyl group, a C₁-C₃-haloalkyl group, a (C₃-C₆-cycloalkyl)-(C₁-C₃-alkyl)- group, a heterocycloalkyl group, a heteroaryl group and a phenyl group,
   wherein said C₃-C₆-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁a C₁-C₃-haloalkyl group, a C₁-C₃-alkoxy group, a C₁-C₃-haloalkoxy group and a R⁵R⁶N— group
   wherein at least one of R^{a} or R^{b} is not a hydrogen atom;
      or a tautomer, or an N-oxide, or a salt thereof, or a salt of a tautomer, or a salt of an N-oxide, or a mixture of same.

In some embodiments, the present invention provides compounds of formula (I), supra, in which
R¹ is selected from a halogen atom and a cyano group;
R² is a —NR^{a}R^{b} group,
   wherein R^{a} and R^{b} together with the nitrogen atom to which they are attached form an azetidine ring, a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
      said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—
      wherein said 4- to 9-membered nitrogen-containing monocyclic, 5- to 11-membered nitrogen-containing bicyclic heterocycloalkyl group, 7- to 9-membered nitrogen-containing bridged compound or 7- to 12-membered nitrogen-containing spiro compound each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo,
      wherein when R^{a} and R^{b} together with the nitrogen atom to which they are attached form a pyrrolidine ring, a piperidine ring or a piperazine ring, said pyrrolidine, piperidine or piperazine ring is not substituted in any of its carbon atoms;
         or a tautomer, or an N-oxide, or a salt thereof, or a salt of a tautomer, or a salt of an N-oxide, or a mixture of same.

In some embodiments, the present invention provides compounds of formula (I), supra, in which
R¹ is selected from a halogen atom and a cyano group;
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group, a 7- to 9-membered nitrogen-containing bridged compound or a 7- to 12-membered nitrogen-containing spiro compound,
      said 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—
      wherein said azetidine ring, morpholine ring, 5- to 11-membered nitrogen-containing bicyclic heterocycloalkyl group, 7- to 9-membered nitrogen-containing bridged compound or 7- to 12-membered nitrogen-containing spiro compound each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
         or a tautomer, or an N-oxide, or a salt thereof, or a salt of a tautomer, or a salt of an N-oxide, or a mixture of same.

In some embodiments, the present invention provides compounds of formula (I), supra, in which
R¹ is selected from a halogen atom and a cyano group;
R² is a —NR^{a}R^{b} group,
   wherein R^{a} and R^{b} together with the nitrogen atom to which they are attached form an azetidine ring, a 4- to 9-membered nitrogen-containing monocyclic or 5- to 11-membered nitrogen-containing bicyclic heterocycloalkyl group,
      said 4- to 9-membered nitrogen-containing monocyclic or 5- to 11-membered nitrogen-containing bicyclic heterocycloalkyl group each optionally containing one, two or three further heteroatoms independently selected from nitrogen, oxygen and sulfur or one group selected from —NR⁸—, —S(═O)—, —S(═O)₂— and —S(═O)(═NH)—
      wherein said 4- to 9-membered nitrogen-containing monocyclic or 5- to 11-membered nitrogen-containing bicyclic heterocycloalkyl group each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo,
      wherein when R^{a} and R^{b} together with the nitrogen atom to which they are attached form a pyrrolidine ring, a piperidine ring or a piperazine ring, said pyrrolidine, piperidine or piperazine ring is not substituted in any of its carbon atoms;
         or a tautomer, or an N-oxide, or a salt thereof, or a salt of a tautomer, or a salt of an N-oxide, or a mixture of same.

In some embodiments, the present invention provides compounds of formula (I), supra, in which
R¹ is selected from a halogen atom and a cyano group;
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring, a morpholine ring, a 5- to 11-membered nitrogen-containing bicyclic heterocycloalkyl group,
      said 5- to 11-membered nitrogen-containing bicyclic heterocycloalkyl group optionally containing one, two or three further heteroatoms independently selected from nitrogen, oxygen and sulfur or one group selected from —NR⁸—, —S(═O)—, —S(═O)₂— and —S(═O)(═NH)—
      said azetidine ring, morpholine ring, 5- to 11-membered nitrogen-containing bicyclic heterocycloalkyl group each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
         or a tautomer, or an N-oxide, or a salt thereof, or a salt of a tautomer, or a salt of an N-oxide, or a mixture of same.

In some embodiments, the present invention provides compounds of formula (I), supra, in which
R¹ is selected from a halogen atom and a cyano group;
R² is a —NR^{a}R^{b} group,
   wherein R^{a} and R^{b} together with the nitrogen atom to which they are attached form a 4- to 9-membered nitrogen-containing monocyclic heterocycloalkyl group,
      said 4- to 9-membered nitrogen-containing monocyclic heterocycloalkyl group optionally containing one, two or three further heteroatoms independently selected from nitrogen, oxygen and sulfur or one group selected from —NR⁸—, —S(═O)—, —S(═O)₂— and —S(═O)(═NH)—
      said 4- to 9-membered nitrogen-containing monocyclic heterocycloalkyl group optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo,
      wherein when R^{a} and R^{b} together with the nitrogen atom to which they are attached form a pyrrolidine ring, a piperidine ring or a piperazine ring, said pyrrolidine, piperidine or piperazine ring is not substituted in any of its carbon atoms;
         or a tautomer, or an N-oxide, or a salt thereof, or a salt of a tautomer, or a salt of an N-oxide, or a mixture of same.

In some embodiments, the present invention provides compounds of formula (I), supra, in which
R¹ is selected from a halogen atom and a cyano group;
R² is a —NR^{a}R^{b} group,
   wherein R^{a} and R^{b} together with the nitrogen atom to which they are attached form an azetidine ring, an unsubstituted pyrrolidine ring, an unsubstituted piperidine ring, an unsubstituted piperazine ring, a morpholine ring,
      wherein said azetidine ring, morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
         or a tautomer, or an N-oxide, or a salt thereof, or a salt of a tautomer, or a salt of an N-oxide, or a mixture of same.

In some embodiments, the present invention provides compounds of formula (I), supra, in which
R¹ is selected from a C₁-C₆-alkyl group, a C₁-C₆-haloalkyl group, a C₃-C₈-cycloalkyl group, a (C₃-C₈-cycloalkyl)-(C₁-C₆-alkyl)- group,
R² is a —NR^{a}R^{b} group,
   or R^{a} and R^{b} together with the nitrogen atom to which they are attached form a morpholine ring, a 5- to 11-membered nitrogen-containing bicyclic heterocycloalkyl group, a 7- to 9-membered nitrogen-containing bridged compound or a 7- to 12-membered nitrogen-containing spiro compound,
      said 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—
   wherein said morpholine ring, 5- to 11-membered nitrogen-containing bicyclic heterocycloalkyl group, 7- to 9-membered nitrogen-containing bridged compound or 7- to 12-membered nitrogen-containing spiro compound each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo
      or a tautomer, or an N-oxide, or a salt thereof, or a salt of a tautomer, or a salt of an N-oxide, or a mixture of same.

In some embodiments, the present invention provides compounds of formula (I), supra, in which
R¹ is selected from a C₁-C₆-alkyl group, a C₁-C₆-haloalkyl group, a C₃-C₈-cycloalkyl group, a (C₃-C₈-cycloalkyl)-(C₁-C₆-alkyl)- group,
R² is a —NR^{a}R^{b} group,
   or R^{a} and R^{b} together with the nitrogen atom to which they are attached form a morpholine ring,
   said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo
      or a tautomer, or an N-oxide, or a salt thereof, or a salt of a tautomer, or a salt of an N-oxide, or a mixture of same.

In some embodiments, the present invention provides compounds of formula (I), supra, in which X is selected from a nitrogen atom and a CR⁴ group or a tautomer, or an N-oxide, or a salt thereof, or a salt of a tautomer, or a salt of an N-oxide, or a mixture of same.

In some embodiments, the present invention provides compounds of formula (I), supra, in which X is a nitrogen atom or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a phenyl group and a heteroaryl group,
wherein said phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group
and wherein, when substituted, said phenyl group is preferably substituted in one or more of the ortho- and/or meta-positions with respect to the point of attachment of said phenyl group to the rest of the molecule
   or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a phenyl group and a heteroaryl group,
wherein said phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group
and wherein, when substituted, said phenyl group is substituted in one or more of the ortho- and/or meta-positions with respect to the point of attachment of said phenyl group to the rest of the molecule
   or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group
   or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a phenyl group and a heteroaryl group,
wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
and wherein, when substituted, said phenyl group is preferably substituted in one or more of the ortho- and/or meta-positions with respect to the point of attachment of said phenyl group to the rest of the molecule
   or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a phenyl group and a heteroaryl group,
wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
and wherein, when substituted, said phenyl group is substituted in one or more of the ortho- and/or meta-positions with respect to the point of attachment of said phenyl group to the rest of the molecule
   or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a phenyl group and a heteroaryl group,
wherein said phenyl or heteroaryl group is each optionally substituted with one or more substituents independently selected from a halogen atom, a cyano group, a hydroxy group, a C₁-C₃-alkyl group, a C₁-C₃-haloalkyl group, a C₁-C₃-alkoxy group, a C₁-C₃-haloalkoxy group,
and wherein, when substituted, said phenyl group is preferably substituted in one or more of the ortho- and/or meta-positions with respect to the point of attachment of said phenyl group to the rest of the molecule
   or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a phenyl group and a heteroaryl group,
wherein said phenyl or heteroaryl group is each optionally substituted with one or more substituents independently selected from a halogen atom, a cyano group, a hydroxy group, a C₁-C₃-alkyl group, a C₁-C₃-haloalkyl group, a C₁-C₃-alkoxy group, a C₁-C₃-haloalkoxy group,
and wherein, when substituted, said phenyl group is substituted in one or more of the ortho- and/or meta-positions with respect to the point of attachment of said phenyl group to the rest of the molecule
   or a tautomer, or an N-oxide, or a salt thereof, or a salt of a tautomer, or a salt of an N-oxide, or a mixture of same.

In some embodiments, the present invention provides compounds of formula (I), supra, in which R³ is selected from a phenyl group and a heteroaryl group,
wherein said phenyl or heteroaryl group is each optionally substituted with one or more substituents independently selected from a halogen atom, a cyano group, a hydroxy group, a C₁-C₃-alkyl group, a C₁-C₃-haloalkyl group, a C₁-C₃-alkoxy group, a C₁-C₃-haloalkoxy group,
and wherein, when substituted, said phenyl group is substituted in one of the ortho- or meta-positions with respect to the point of attachment of said phenyl group to the rest of the molecule
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R⁴ is selected from a hydrogen atom, a C₁-C₃-alkyl group and a C₁-C₃-haloalkyl group;

or R³ and R⁴, together with the carbon atoms to which they are attached form a 5- to 7-membered cycloalkyl, cycloalkenyl, heterocycloalkyl, heterocycloalkenyl, phenyl or heteroaryl group,
wherein said heterocycloalkyl, heterocycloalkenyl or heteroaryl group contains one or two heteroatoms independently selected from nitrogen, oxygen and sulfur,
and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R⁴ is selected from a hydrogen atom, a methyl group and a trifluoromethyl group;

or R³ and R⁴, together with the carbon atoms to which they are attached form a 5- to 7-membered cycloalkyl, cycloalkenyl, heterocycloalkyl, heterocycloalkenyl, phenyl or heteroaryl group,
wherein said heterocycloalkyl, heterocycloalkenyl or heteroaryl group contains one or two heteroatoms independently selected from nitrogen, oxygen and sulfur,
and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R⁴ is selected from a hydrogen atom, a methyl group and a trifluoromethyl group;

or R³ and R⁴, together with the carbon atoms to which they are attached form a 6-membered cycloalkyenl, phenyl or heteroaryl group,
wherein said or heteroaryl group contains one or two heteroatoms independently selected from nitrogen, oxygen and sulfur,
and wherein said cycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₆-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R⁴ is selected from a hydrogen atom, a methyl group and a trifluoromethyl group;

or R³ and R⁴, together with the carbon atoms to which they are attached form a 6-membered cycloalkenyl group, a phenyl group or a 6-membered heteroaryl group,
wherein said or heteroaryl group contains one or two heteroatoms independently selected from nitrogen, oxygen and sulfur,
and wherein said cycloalkenyl, phenyl or heteroaryl group is each optionally substituted one, two or three times, each substituent independently selected from a halogen atom, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group, a C₃-C₅-cycloalkyl group, a C₃-C₆-cycloalkoxy group, a R⁵R⁶N— group, a (R⁵R⁶N)—(C₁-C₆-alkyl)- group,
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R⁴ is selected from a hydrogen atom, a C₁-C₃-alkyl group and a C₁-C₃-haloalkyl group;

or R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R⁴ is selected from a hydrogen atom, a methyl group and a trifluoromethyl group;

or R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R⁴ is selected from a hydrogen atom, a C₁-C₃-alkyl group and a C₁-C₃-haloalkyl group or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R⁴ is selected from a hydrogen atom, a methyl group and a trifluoromethyl group or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a 5- to 7-membered cycloalkenyl, heterocycloalkenyl, phenyl or heteroaryl group,
wherein said heterocycloalkenyl or heteroaryl group contains one or two heteroatoms independently selected from nitrogen, oxygen and sulfur,
and wherein said cycloalkenyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a 6-membered cycloalkenyl group, a phenyl group or a 6-membered heteroaryl group,
wherein said heteroaryl group contains one or two heteroatoms independently selected from nitrogen, oxygen and sulfur,
and wherein said cycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₆-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a 6-membered cycloalkenyl or 6-membered heterocycloalkenyl group,
wherein said 6-membered heterocycloalkenyl group contains one or two heteroatoms independently selected from nitrogen, oxygen and sulfur,
and wherein said 6-membered cycloalkenyl or 6-membered heterocycloalkenyl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a 6-membered cycloalkenyl or 6-membered heterocycloalkenyl group,
wherein said 6-membered heterocycloalkenyl group contains one or two heteroatoms independently selected from nitrogen, oxygen and sulfur,
and wherein said 6-membered cycloalkenyl or 6-membered heterocycloalkenyl group is each optionally substituted one, two or three times, each substituent independently selected from a halogen atom, a C₁-C₃-alkyl group, a C₁-hydroxyalkyl group, a C₁-C₃-haloalkyl group, a C₁-C₃-alkoxy group, a C₁-C₆-haloalkoxy group and a C₃-C₆-cycloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a 6-membered cycloalkenyl or 6-membered heterocycloalkenyl group,
wherein said 6-membered heterocycloalkenyl group contains one or two heteroatoms independently selected from nitrogen, oxygen and sulfur,
and wherein said 6-membered cycloalkenyl or 6-membered heterocycloalkenyl group is each optionally substituted one or two times, each substituent independently selected from a halogen atom, a C₁-C₃-alkyl group, a C₁-hydroxyalkyl group, a C₁-C₃-haloalkyl group, a C₁-C₃-alkoxy group and a C₁-C₆-haloalkoxy group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a 6-membered cycloalkenyl group,
wherein said 6-membered cycloalkenyl group is each optionally substituted one or two times, each substituent independently selected from a halogen atom, a C₁-C₃-alkyl group, a C₁-hydroxyalkyl group, a C₁-C₃-haloalkyl group, a C₁-C₃-alkoxy group and a C₁-C₆-haloalkoxy group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl or a heteroaryl group,
wherein said heteroaryl group contains one or two heteroatoms independently selected from nitrogen, oxygen and sulfur,
and wherein said phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl group,
wherein said phenyl group is optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group, a C₃-C₆-cycloalkoxy group, a R⁵R⁶N— group, a (R⁵R⁶N)—(C₁-C₆-alkyl)- group, and a R⁷OOC— group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl group,
wherein said phenyl group is optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₆-cycloalkyl group and a C₃-C₆-cycloalkoxy group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl group,
wherein said phenyl group is optionally substituted one, two or three times, each substituent independently selected from a halogen atom, a cyano group, a C₁-C₆-alkyl group, a C₁-C₆-alkoxy group, a C₁-C₆-hydroxyalkyl group, a C₁-C₆-haloalkyl group, and a C₃-C₆-cycloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl group,
wherein said phenyl group is optionally substituted one, two or three times, each substituent independently selected from a halogen atom, a C₁-C₆-alkyl group, a C₁-C₆-alkoxy group, a C₁-C₆-hydroxyalkyl group, a C₁-C₆-haloalkyl group, and a C₃-C₆-cycloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl group,
wherein said phenyl group is optionally substituted one or two times, each substituent independently selected from a halogen atom, a C₁-C₆-alkyl group, a C₁-C₆-alkoxy group, a C₁-C₆-hydroxyalkyl group and a C₁-C₆-haloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which X is a CR⁴ group and R³ and R⁴, together with the carbon atoms to which they are attached form a phenyl group,
wherein said phenyl group is optionally substituted one or two times, each substituent independently selected from a halogen atom, a C₁-C₃-alkyl group, a C₁-C₃-alkoxy group, a C₁-C₃-hydroxyalkyl group and a C₁-C₃-haloalkyl group;
   or a tautomer, or an N-oxide, or a salt thereof, or a salt of a tautomer, or a salt of an N-oxide, or a mixture of same.

In some embodiments, the present invention provides compounds of formula (I), supra, in which R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group or a tautomer, or an N-oxide, or a salt thereof, or a salt of a tautomer, or a salt of an N-oxide, or a mixture of same.

In some embodiments, the present invention provides compounds of formula (I), supra, in which R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heteroaryl group and a phenyl group or a tautomer, or an N-oxide, or a salt thereof, or a salt of a tautomer, or a salt of an N-oxide, or a mixture of same.

In some embodiments, the present invention provides compounds of formula (I), supra, in which R⁵ and R⁶ are each independently selected from a hydrogen atom, a C₁-C₃-alkyl group, a C₃-C₈-cycloalkyl group, a C₁-C₃-haloalkyl group, a (C₃-C₈-cycloalkyl)-(C₁-C₃-alkyl)- group, a C₁-C₃-hydroxyalkyl group, a (C₁-C₃-alkoxy)-(C₁-C₃-alkyl)- group, a heteroaryl group and a phenyl group or a tautomer, or an N-oxide, or a salt thereof, or a salt of a tautomer, or a salt of an N-oxide, or a mixture of same.

In some embodiments, the present invention provides compounds of formula (I), supra, in which R⁵ and R⁶ are each independently selected from a hydrogen atom, a C₁-C₃-alkyl group, a C₃-C₈-cycloalkyl group, a C₁-C₃-haloalkyl group, a (C₃-C₈-cycloalkyl)-(C₁-C₃-alkyl)- group, a C₁-C₃-hydroxyalkyl group, a (C₁-C₃-alkoxy)-(C₁-C₃-alkyl)- group, or a tautomer, or an N-oxide, or a salt thereof, or a salt of a tautomer, or a salt of an N-oxide, or a mixture of same.

In some embodiments, the present invention provides compounds of formula (I), supra, in which R⁵ and R⁶ are each independently selected from a hydrogen atom, a C₁-C₃-alkyl group, a C₃-C₆-cycloalkyl group, a C₂-C₃-haloalkyl group, a (C₃-C₆-cycloalkyl)-(C₁-C₃-alkyl)- group, a C₂-C₃-hydroxyalkyl group, a (C₁-C₃-alkoxy)-(C₁-C₃-alkyl)- group, or a tautomer, or an N-oxide, or a salt thereof, or a salt of a tautomer, or a salt of an N-oxide, or a mixture of same.

In some embodiments, the present invention provides compounds of formula (I), supra, in which R⁵ and R⁶ are each independently selected from a hydrogen atom, a heteroaryl group and a phenyl group or a tautomer, or an N-oxide, or a salt thereof, or a salt of a tautomer, or a salt of an N-oxide, or a mixture of same.

In some embodiments, the present invention provides compounds of formula (I), supra, in which R⁷ is selected from a hydrogen atom and a C₁-C₃-alkyl group or a tautomer, or an N-oxide, or a salt thereof, or a salt of a tautomer, or a salt of an N-oxide, or a mixture of same.

In some embodiments, the present invention provides compounds of formula (I), supra, in which R⁷ is a hydrogen atom or a tautomer, or an N-oxide, or a salt thereof, or a salt of a tautomer, or a salt of an N-oxide, or a mixture of same.

In some embodiments, the present invention provides compounds of formula (I), supra, in which R⁷ is a C₁-C₃-alkyl group or a tautomer, or an N-oxide, or a salt thereof, or a salt of a tautomer, or a salt of an N-oxide, or a mixture of same.

In some embodiments, the present invention provides compounds of formula (I), supra, in which R⁸ is selected from a hydrogen atom, a C₁-C₆-alkyl group, a C₃-C₆-cycloalkyl group and a C₁-C₆-haloalkyl group or a tautomer, or an N-oxide, or a salt thereof, or a salt of a tautomer, or a salt of an N-oxide, or a mixture of same.

In some embodiments, the present invention provides compounds of formula (I), supra, in which R⁸ is selected from a hydrogen atom, a C₁-C₃-alkyl group, a C₃-C₆-cycloalkyl group and a C₁-C₃-haloalkyl group or a tautomer, or an N-oxide, or a salt thereof, or a salt of a tautomer, or a salt of an N-oxide, or a mixture of same.

In some embodiments, the present invention provides compounds of formula (I), supra, in which R^{a} is selected from a hydrogen atom, a C₁-C₆-alkyl group, or a tautomer, or an N-oxide, or a salt thereof, or a salt of a tautomer, or a salt of an N-oxide, or a mixture of same.

In some embodiments, the present invention provides compounds of formula (I), supra, in which R^{a} is a hydrogen atom, or a tautomer, or an N-oxide, or a salt thereof, or a salt of a tautomer, or a salt of an N-oxide, or a mixture of same.

In some embodiments, the present invention provides compounds of formula (I), supra, in which R⁸ is a C₁-C₆-alkyl group, or a tautomer, or an N-oxide, or a salt thereof, or a salt of a tautomer, or a salt of an N-oxide, or a mixture of same.

In some embodiments, the present invention provides compounds of formula (I), supra, in which R^{a} is a C₁-C₃-alkyl group, or a tautomer, or an N-oxide, or a salt thereof, or a salt of a tautomer, or a salt of an N-oxide, or a mixture of same.

In some embodiments, the present invention provides compounds of formula (I), supra, in which R^{a} is a methyl group, or a tautomer, or an N-oxide, or a salt thereof, or a salt of a tautomer, or a salt of an N-oxide, or a mixture of same.

In further embodiments, the present invention includes compounds of formula (I), or a tautomer, an N-oxide, or a salt thereof, or a salt of a tautomer or an N-oxide, or a mixture of same.

In further embodiments, the present invention includes compounds of formula (I), or a salt thereof.

In further embodiments, the present invention includes compounds of formula (I), or a tautomer, or a salt thereof, or a salt of a tautomer, or a mixture of same.

In further embodiments, the present invention includes compounds of formula (I), which are a salt.

In further embodiments, the present invention includes compounds of formula (I), which are a tautomer or a salt thereof, or a salt of a tautomer, or a mixture of same.

In further embodiments, the present invention includes compounds of formula (I), which are an N-oxide, or a salt thereof, or a salt of an N-oxide, or a mixture of same.

In further embodiments of the first aspect, the present invention provides combinations of two or more of the above mentioned embodiments under the heading “further embodiments of the first aspect of the present invention”.

Furthermore it is understood that the invention includes any subcombination of the disclosed single embodiments herein for certain residues or subcombination of residues of formula (I).

The present invention includes any sub-combination within any embodiments or aspects of the present invention of compounds of general formula (I), supra.

The present invention includes any sub-combination within any embodiments or aspects of the present invention of compounds of general formula (I) or intermediate compounds.

The present invention includes the compounds of general formula (I) which are disclosed in the Example Section of this text, infra.

### General Synthesis of Compounds of General Formula (I) of the Present Invention

The following paragraphs outline a variety of synthetic approaches suitable to prepare compounds of the general formula (I), and intermediates useful for their synthesis.

In addition to the routes described below, also other routes may be used to synthesise the target compounds, in accordance with common general knowledge of a person skilled in the art of organic synthesis. The order of transformations exemplified in the following schemes is therefore not intended to be limiting, and suitable synthesis steps from various schemes can be combined to form additional synthesis sequences. In addition, interconversion of any of the substituents, in particular R¹, R², R³ or R⁴ can be achieved before and/or after the exemplified transformations. These modifications can be such as the introduction of protective groups, cleavage of protective groups, reduction or oxidation of functional groups, halogenation, metallation, metal catalysed coupling reactions, exemplified by but not limited to Suzuki, Sonogashira and Ullmann coupling, ester saponifications, amide coupling reactions, and/or substitution or other reactions known to a person skilled in the art. These transformations include those which introduce a functionality allowing for further interconversion of substituents. Appropriate protective groups and their introduction and cleavage are well-known to a person skilled in the art (see for example T. W. Greene and P. G. M. Wuts in Protective Groups in Organic Synthesis, 3^{rd} edition, Wiley 1999).

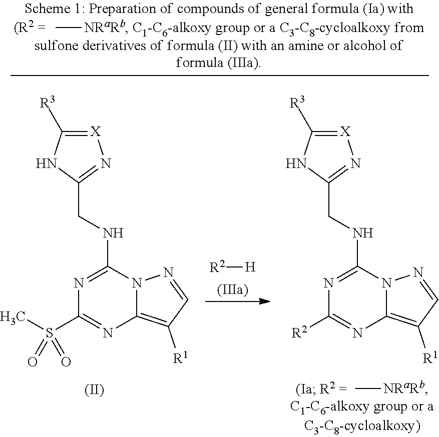

Compounds of general formula (Ia), in which R¹, R³ and X are as defined for the compounds of general formula (I) and R² is —NR^{a}R^{b}, C₁-C₆-alkoxy group or a C₃-C₈-cycloalkoxy, can be assembled from sulfone derivatives of formula (II), in which R¹, R³ and X are as defined for the compounds of general formula (I), and an amine or an alcohol of formula R²—H (IIIa), in which R² is defined as —NR^{a}R^{b}, C₁-C₆-alkoxy or a C₃—C-cycloalkoxy, by means of an aromatic nucleophilic substitution well known to the person skilled in the art, according to Scheme 1. Said nucleophilic reaction can be performed by reaction of compounds of the formulae (II) and (IIIa) in the presence of a suitable base, such as sodium hydroxide, sodium hydride, sodium carbonate, potassium carbonate or cesium carbonate, N,N-diisopropylethylamine, triethylamine or 1,8-diazabicyclo(5.4.0)undec-7-ene (DBU), and in the case of aromatic amines in the presence of an acid such as 4-methylbenzenesulfonic acid in an appropriate solvent.

Preferred herein is the performance of said nucleophilic reaction in the case of amines using N,N-diisopropylethylamine as a base in acetonitrile as a solvent, within a temperature range from 20° C. to 80° C.

Also preferred herein is the performance of said nucleophilic reaction in the case of aromatic amines using 4-methylbenzenesulfonic acid in N-methyl-2-pyrrolidone (NMP) as a solvent, within a temperature range from 100° C. to 170° C.

Compounds of the general formula (I) in which R¹ is a cyano group, a phenyl group, or a heteroaryl group can be assembled from a corresponding compound of the general formula (I) in which R¹ is chloro, bromo or iodo and free NH- groups may be protected for example with a para-methoxybenzyl group using palladium catalyst reactions.

For a cyano group for example the corresponding bromide reacts with zinc cyanide in the presence of 1,1′-bis(diphenylphosphanyl)ferrocene and N,N-diisopropylethylamine in an appropriate solvent such as N,N-dimethylacetamide within a temperature range from 60° C. to 90° C.

For a phenyl group or a heteroaryl group the corresponding bromide reacts via a Suzuki reaction using a corresponding boronic acid derivative in the presence of a Pd-catalyst and a base in an appropriate solvent.

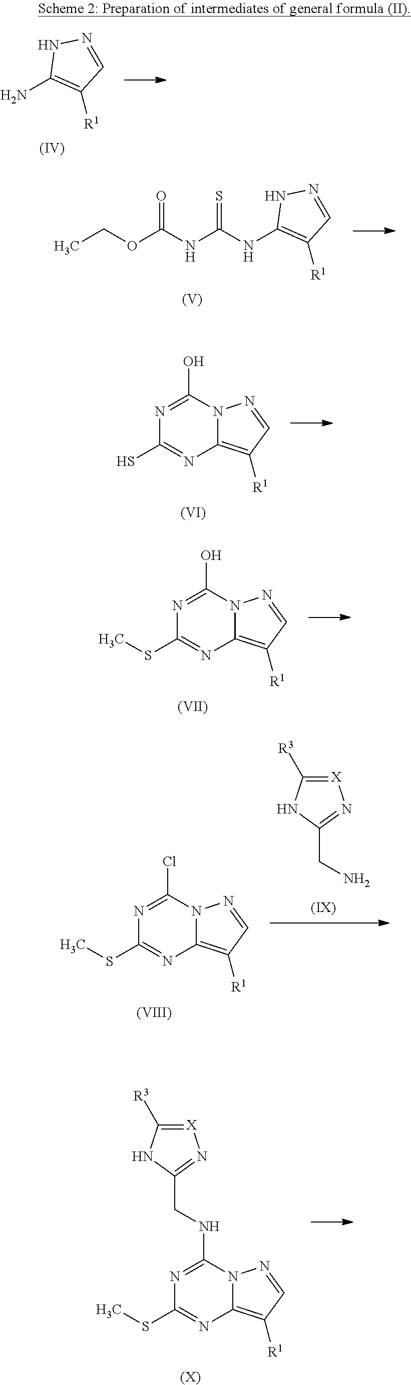

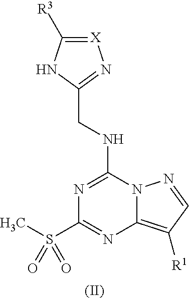

Intermediate sulfone derivatives of formula (II) are available for example by the sequence depicted in scheme 2. This approach started with commercially available or synthesized (according to e.g. WO2018/195397) amino-pyrazole derivatives of the formula (IV), in which R¹ are as defined for the compounds of general formula (I), with ethyl carbonisothiocyanatidate in ethyl acetate to give intermediates (V), which under basic condition such as aqueous sodium hydroxide form the pyrazolotriazin derivatives of the formula (VI). Using methyliodide under basic conditions such as sodium hydroxide the methylsulfanyl derivatives (VII) are formed. In the case of R¹═H in compound (VII) it is possible to introduce halogens such as bromo, chloro or iodo using the corresponding N-halo-succinimide reagent. Reaction of said derivatives (VII) with phosphorus oxychloride resulted in chloro intermediates (VIII). Reaction of (VIII) with commercially available or prepared amines of the general formula (IX) in which R³ and X are as defined for the compounds of general formula (I) under basic condition such as N,N-diisopropylethylamine in an appropriate solvent such as acetonitrile within a temperature range from 20° C. to 80° C. and the subsequent oxidation of the sulfur atom with meta-chloroperoxybenzoic acid (mCPBA) yields the sulfones (II).

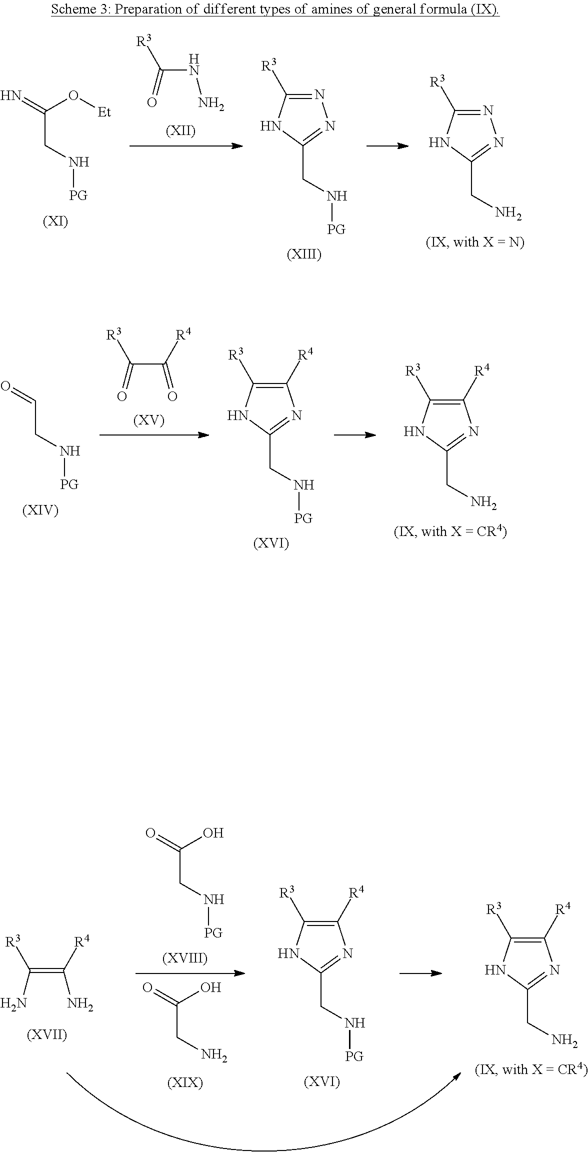

The synthesis of different types of amines of the general formula (IX) is depicted in scheme 3. In the case of amines with X═N and R³ is as defined for the compounds of general formula (I) in the first step commercially available protected ethyl 2-aminoethanimidate (XI) reacts with acylhydrazides of the general formula (XII) in which R³ is as defined for the compounds of general formula (I) according to US2010/22599 under basic conditions such as sodium bicarbonate or potassium carbonate to yield the protected amines (XIII) which in the subsequent step are deprotected using conditions known by the person skilled in the art to give amines of formula (IX) with X═N. The used acylhydrazides (XII) are commercially available or can be easily prepared using the corresponding acid or ester via known procedures for person skilled in the art.

For amines (IX) with X═CR⁴ and R⁴ as defined for the compounds of general formula (I) commercially available protected aminoacetaldehydes (XIV) react with 1,2-diketones (XV) (for preparation see Landais, Y.; Vincent, J. M., Science of Synthesis, (2005) 26, 647) in the presence of ammonium acetate in methanol/tetrahydrofurane according to Bioorganic and Medicinal Chemistry, 2012, 7128 to yield the protected amines (XVI) which in the subsequent step are deprotected unsing conditions known by the person skilled in the art to give amines of formula (IX) with X═CR⁴.

These amines can be also prepared starting with 1,2-diamino compounds (XVII) by the reaction with commercially available protected glycine derivatives of formula (XVIII) using 1-ethyl-3-(3-dimethylaminopropyl)carbodiimide hydrochloride and hydroxybenzotriazole mono hydrate followed by acetic acid according to Bioorganic and Medicinal Chemistry Letters, 2013, 4374 to yield the protected amines (XVI) which in the subsequent step are deprotected unsing conditions known by the person skilled in the art to give amines of formula (IX) with X═CR⁴.

Alternatively, the 1,2-diamino compounds (XVII) can react with glycine (XIX) using acid condition such as aqueous HCl according to EP1135374 (2006) to give amines of formula (IX) with X═CR⁴.

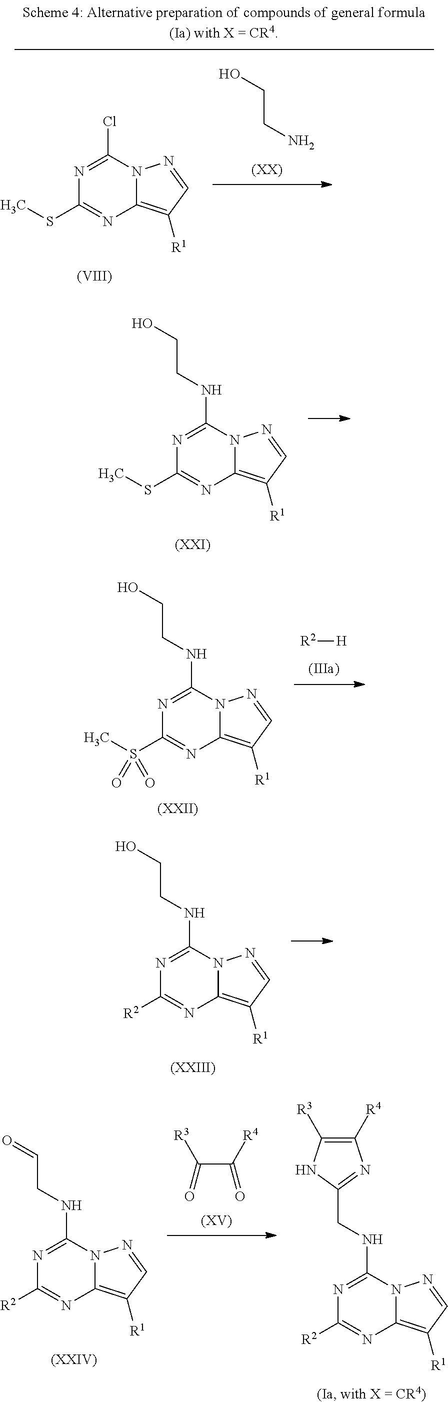

Alternatively, compounds of the formula (VIII) can react with 2-aminoethanol (XX) to give compounds of formula (XXI) which can be oxidized with meta-chloroperoxybenzoic acid (mCPBA) to sulfones of the formula (XXII). These sulfones of formula (XXII) and an amine or an alcohol of formula R²—H (IIIa), in which R² is defined as —NR^{a}R^{b}, C₁-C₈-alkoxy or a C₃-C₈-cycloalkoxy, can react in an aromatic nucleophilic substitution well known to the person skilled in the art and as described for Scheme 1 to give compounds of the formula (XXIII) which can be oxidized to the corresponding aldehydes of formula (XXIV) with methods well known to the person skilled in the art. Compounds of the formula (Ia) with X═CR⁴ and R⁴ as defined for the compounds of general formula (I) can be assembled by the reaction of aldehydes of formula (XXIV) with 1,2-diketones of formula (XV) as described for scheme 3.

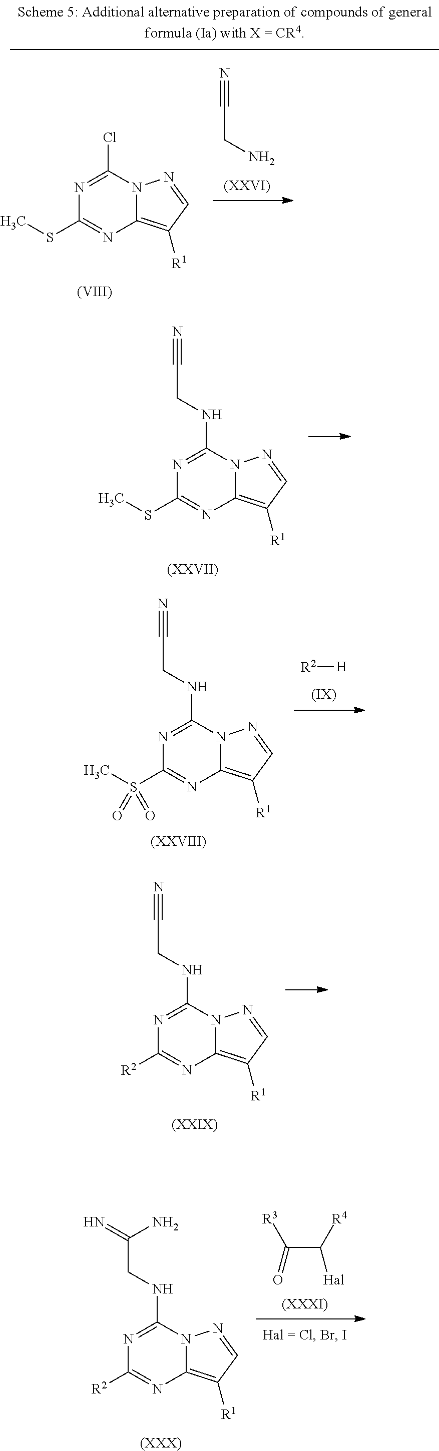

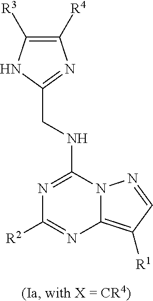

Alternatively, compounds of the formula (VIII) can react with aminoacetonitrile (XXVI) to give compounds of formula (XXVII) which can be oxidized with meta-chloroperoxybenzoic acid (mCPBA) to sulfones of the formula (XXVIII). These sulfones of formula (XXVIII) and an amine or an alcohol of formula R²—H (IIIa), in which R² is defined as —NR^{a}R^{b}, C₁-C₆-alkoxy or a C₃-C₈-cycloalkoxy, can react in an aromatic nucleophilic substitution well known to the person skilled in the art and as described for scheme 1 to give compounds of the formula (XXXIX) which can be reacted to the corresponding imidamides of formula (XXX) with methods well known to the person skilled in the art. Compounds of the formula (Ia) with X═CR⁴ and R⁴ as defined for the compounds of general formula (I) can be assembled by the reaction of imidamides of formula (XXX) with alpha-halogenated ketones of formula (XXXI) with methods well known to the person skilled in the art.

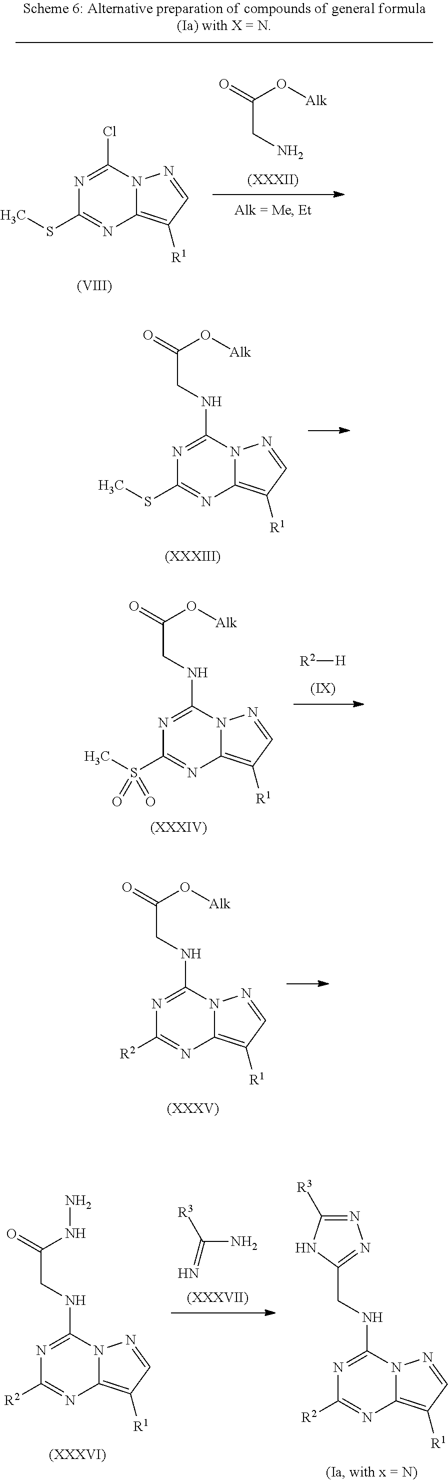

Alternatively, compounds of the formula (VIII) can react with glycinates of formula (XXXII) to give compounds of formula (XXXIII) which can be oxidized with meta-chloroperoxybenzoic acid (mCPBA) to sulfones of the formula (XXXIV). These sulfones of formula (XXXIV) and an amine or an alcohol of formula R²—H (IIIa), in which R² is defined as —NR^{a}R^{b}, C₁-C₆-alkoxy or a C₃-C₈-cycloalkoxy, can react in an aromatic nucleophilic substitution well known to the person skilled in the art and as described for scheme 1 to give compounds of the formula (XXXV) which can be reacted to the corresponding hydrazides of formula (XXXVI) with methods well known to the person skilled in the art. Compounds of the formula (Ia) with X═N and R⁴ as defined for the compounds of general formula (I) can be assembled by the reaction of hydrazides of formula (XXXVI) with imidamides of formula (XXXVII) with methods well known to the person skilled in the art.

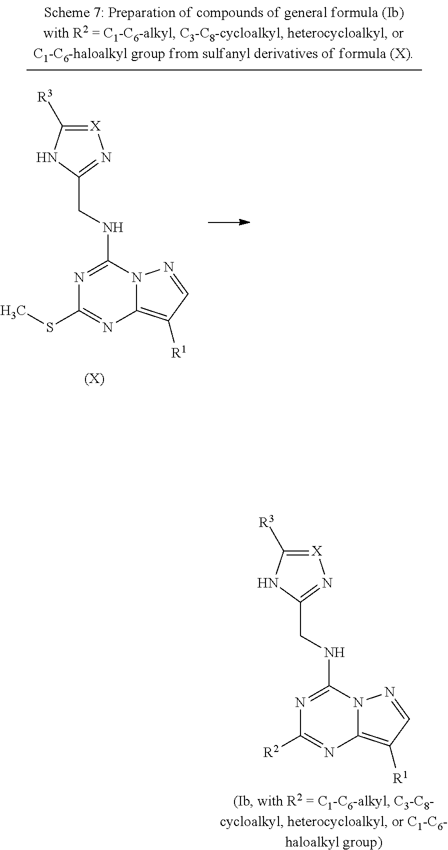

By the following transformations in some cases the intermediate introduction of a protecting group such as p-methoxy-benzyl is important for a better reaction and/or yield. Afterwards these protecting groups are easily cleaved using reagents known to the person skilled in the art.

R²═C₁-C₆-alkyl, C₃-C₈-cycloalkyl or heterocycloalkyl group can be introduced starting from methylsulfanyls of formula (X) via Pd/Cu chemistry using a corresponding boronic acid (for better reactivity the corresponding vinylic boronic acid with subsequent hydrogenation is also possible), for example WO 2018/195397 using a vinylic type of boronic acid in the presence of tetrakis(triphenylphosphine)palladium and copper(I)thiophene-2-carboxylate in a solvent such as tetrahydrofuran at higher temperature such as 100° C.

R²═C₁-C₆-alkyl can be introduced according to Chemical and Pharmaceutical Bulletin 1989, 1731 using the corresponding sulfone (II) and a Grignard reagent with the corresponding alkyl substituent.

R²═C₁-C₆-haloalkyl can be introduced by a multistep sequence in which for example for —CHF₂ a vinyl group is introduced by a Suzuki reaction, subsequent ozonolysis results an aldehyde which then is transformed using for example diethylaminosulfur trifluoride (DAST) as fluorinating agent (e.g. analogous to. US2014/100231). Introduction of —CF₃ can be achieved via the acid by oxidation of the aforementioned aldehyde and subsequent fluorination with DAST or sulfur tetrafluoride reaction.

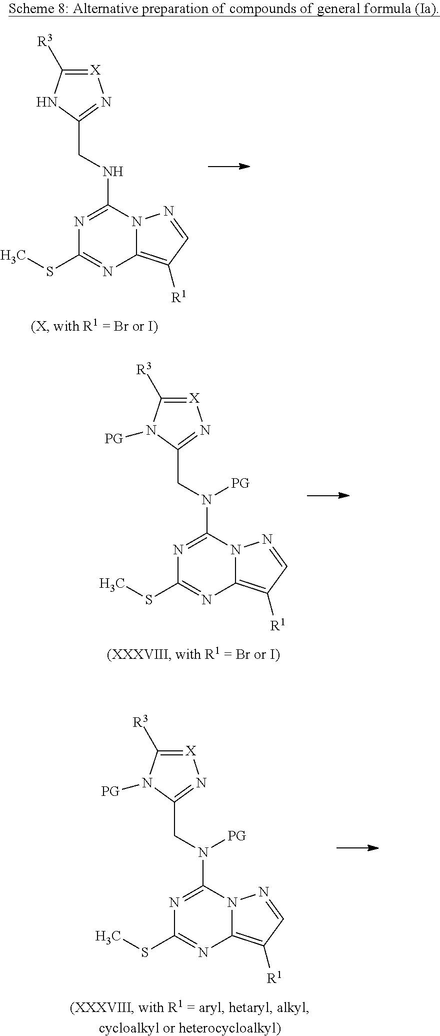

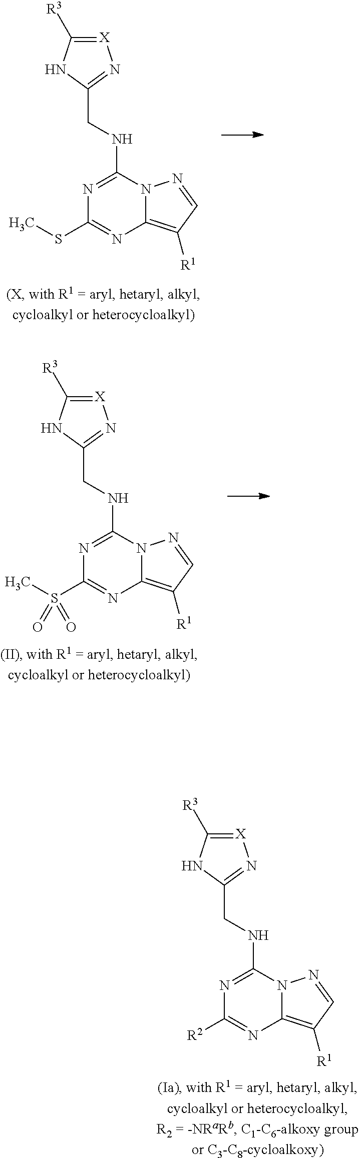

Alternatively, compounds of the formula (X) with R¹ defined as bromine or iodine can be protected with a protecting group (PG) as for example para-methoxybenzyl with methods well known to the person skilled in the art and react via a Suzuki reaction using a corresponding boronic acid derivative in the presence of a Pd-catalyst and a base in an appropriate solvent to give compounds of the formula (XXXVIII). After deprotection with methods well known to the person skilled in the art the methylsulfanyl of formula (X) can be oxidized with meta-chloroperoxybenzoic acid (mCPBA) to sulfones of the formula (II). These sulfones of formula (II) and an amine or an alcohol of formula R²—H (IIIa), in which R² is defined as —NR^{a}R^{b}, C₁-C₆-alkoxy or a C₃-C₈-cycloalkoxy, can react in an aromatic nucleophilic substitution well known to the person skilled in the art and as described for scheme 1 to give compounds of the formula (Ia).

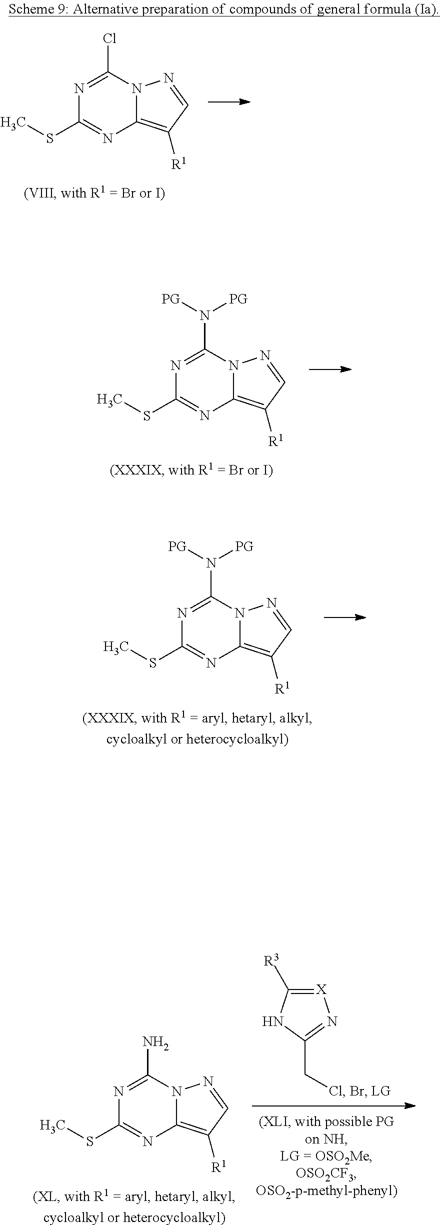

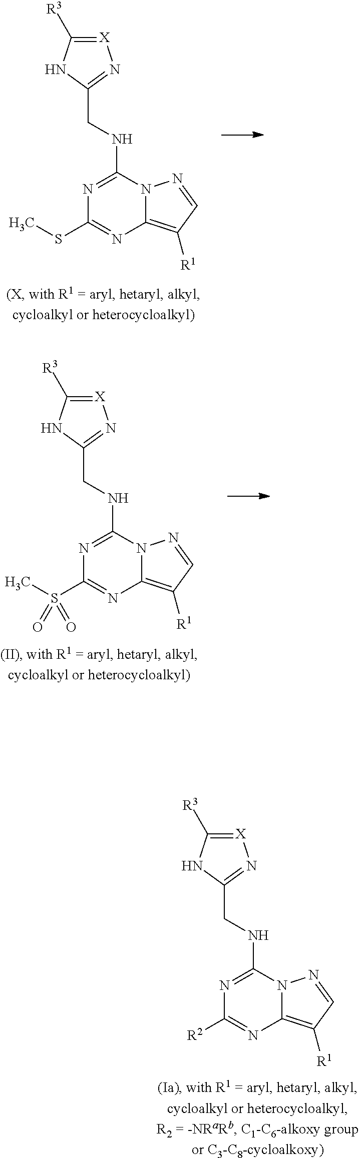

Alternatively, compounds of the formula (VIII) with R¹ defined as bromine or iodine can react with an amine with two protecting groups (PG) as for example para-methoxybenzyl with methods well known to the person skilled in the art and react further via a Suzuki reaction using a corresponding boronic acid derivative in the presence of a Pd-catalyst and a base in an appropriate solvent to give compounds of the formula (XXXIX). After deprotection with methods well known to the person skilled in the art the amine of the formula (XL) can react with a bromide or chloride of the formula (XLI) with methods well known to the person skilled in the art to give compounds of formula (X) which can be oxidized with meta-chloroperoxybenzoic acid (mCPBA) to sulfones of the formula (II). These sulfones of formula (II) and an amine or an alcohol of formula R²—H (IIIa), in which R² is defined as —NR^{a}R^{b}, C₁-C₆-alkoxy or a C₃-C₈-cycloalkoxy, can react in an aromatic nucleophilic substitution well known to the person skilled in the art and as described for scheme 1 to give compounds of the formula (Ia).

Alternatively, compounds of the formula (XXXVIII) or (XXXIX) with R¹ defined as iodine can react with a trifluoromethylating reagent as methyl difluoro(fluorosulfonyl)acetate in the presence of copper(I)iodide in an appropriate solvent to give compounds of the formula (XXXVIII) or (XXXIX) with R¹ defined as a trifluoromethyl group. Afterwards these compounds can react in several steps as described to compounds of the formula (Ia).

In addition to the previous method other trifluoromethylating reagents are useful too, e.g. trimethyl(trifluoromethyl)silane in the presence of potassium fluoride and copper(I)iodide in an inert solvent such as 1-methyl-pyrrolidin-2-one (e.g. WO2014/99836 or Journal of Fluorine Chemistry (2013), 156, 170-176) or triethyl(trifluoromethyl)silane in the presence of potassium fluoride and copper(I)iodide in an inert solvent (e.g. WO2004/022560). Further technologies use trifluoromethylsulfonium salt (e.g. Angewandte Chemie, International Edition (2011), 50(8), 1896-1900) or chlorodifluoroacetic acid derivatives (.e.g. Organic Process Research & Development (2016), 20(4), 836-839, Organic Letters (2015), 17(9), 2086-2089 or Nature Chemistry (2013), 5(11), 941-944) ortrifluoromethane, potassium tert-butoxide and copper(I)chloride (Journal of the American Chemical Society (2011), 133(51), 20901-20913) or iodotrifluoromethane, copper(I)chloride and 1,10-Phenanthroline (JP2013241345) or iodotrifluoromethane using Pd/Zn-chemistry (Chemistry Letters. 11 (1): 137-140) or trifluoromethanesulfonyl chloride in a photochemistry reaction (Nature. 480 (7376): 224-228).

A sequence of Pd-catalyzed carbonylation to a carboxylic ester, saponfication and reaction using fluorinating agent such as diethylaminosulfur trifluoride (DAST) or SF₄ or using Togni's reagent in a photochemistry step (Journal of the American Chemical Society (2018), 140, 6522-6526) is another possible way well known to the person skilled in the art to build up a trifluoromethyl- group.

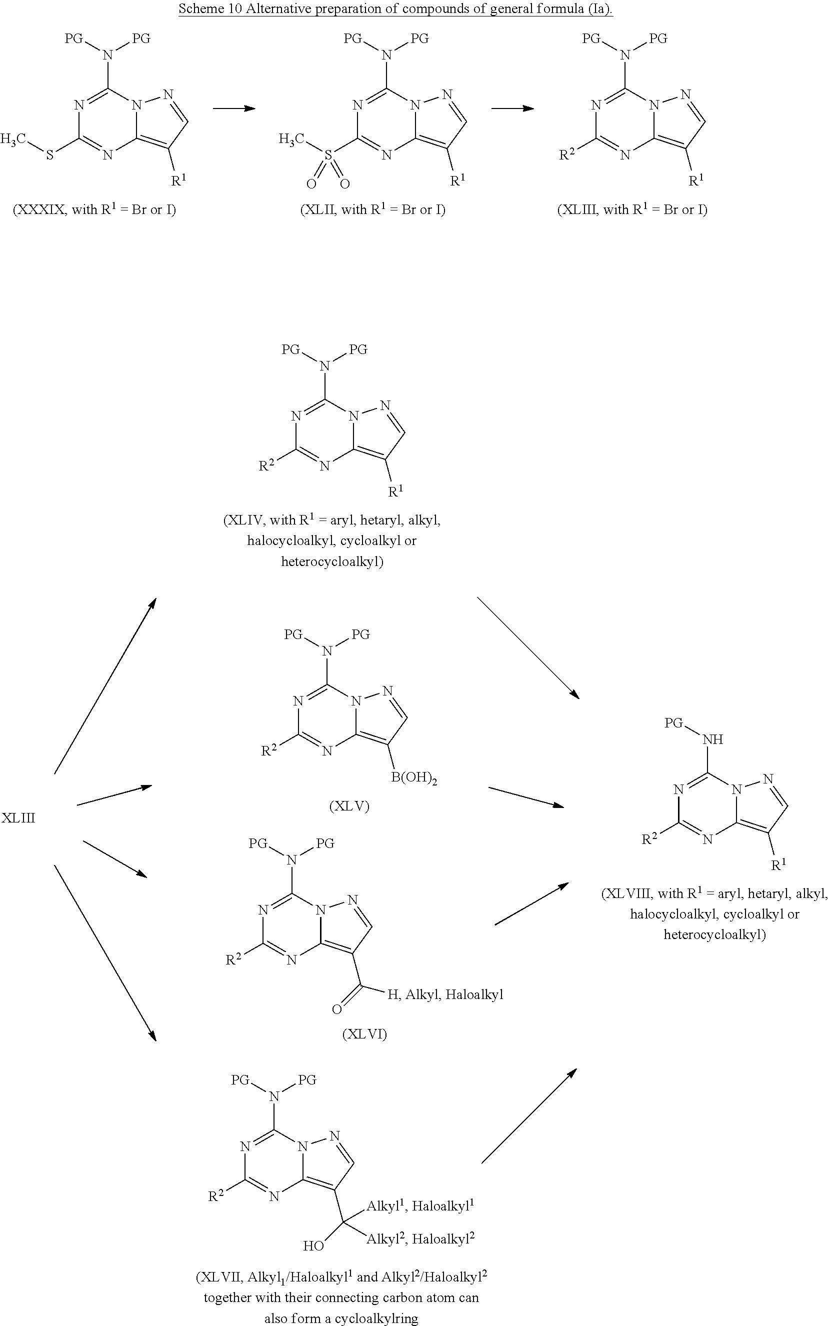

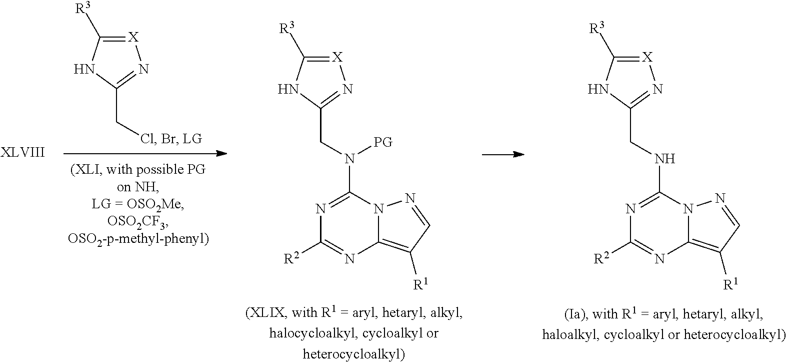

Alternatively, compounds of the formula (XXXIX with R¹ defined as bromine or iodine can be oxidized with meta-chloroperoxybenzoic acid (mCPBA) to sulfones of the formula (XLII). These sulfones of formula (XLII) and an amine or an alcohol of formula R²—H (IIIa) can react in an aromatic nucleophilic substitution well known to the person skilled in the art to compounds of formula (XLIII).

The compounds (XLIII) can then react further via a Suzuki reaction using a corresponding boronic acid derivative in the presence of a Pd-catalyst and a base in an appropriate solvent to compound (XLIV) followed by a deprotection of only one PG- group with e.g. trifluoroacetic acid or other methods well known to the person skilled in the art to give amines of formula (XLVIII). In addition to the aforementioned Suzuki reaction it is possible to transform e.g. the iodine (XLIII) using transmetallation with a Grignard reagent followed by the reaction with trimethyl borate and workup to the corresponding boronic acid (XLV) and then react via Suzuki reaction with an aryl or hetaryl bromide or iodide in the presence of a Pd-catalyst and a base in an appropriate solvent followed by a deprotection of only one PG- group with methods well known to the person skilled in the art to give amines of formula (XLVIII).

In the case of R¹ is alkyl, haloalkyl or cycloalkyl a different sequence is possible using a transmetallation of compounds such as (XLIII) with a Grignard reagent followed by the reaction with a ketone, halogenated ketone or cycloalkanone to give tertiary alcohols of formula (XLVII). These alcohols are then reduced using triethylsilane in the presence of trifluoroacetic acid to yield compounds of formula (XLVIII).

In a similar sequence for R¹ e.g. 2,2,2-trifluoroethyl and similar haloalkyls the transmetallation of compounds (XLIII) with a Grignard reagent followed by the reaction with DMF produces an aldyhyde of formula (XLVI). The reaction of this aldehyde with TMSCF₃ in the presence of CsF or a Grignard reagent produces an alcohol which is reduced by using triethylsilane in the presence of trifluoroacetic acid to give the corresponding compound (XLVIII).

Similarly, for R¹ e.g. 2,2-difluoroethyl a transmetallation of compounds (XLIII) with a Grignard reagent followed by the reaction with a Weinreb amide produces a ketone which is then reduced using triethylsilane in the presence of trifluoroacetic acid to corresponding compounds (XLVIII).

For R¹ e.g. 2,2-difluorocyclopropyl a reaction of the iodine via a Suzuki reaction with potassium ethenyl(trifluorido)borate to give a vinyl compound which reacts with trimethyl (trifluoromethyl)silane and sodium iodate to give the difluorocyclopropyl ring system followed by a deprotection of only one PG- group with e.g. trifluoroacetic acid or other methods well known to the person skilled in the art to give amines of formula (XLVIII).

In addition, such Suzuki reactions with substituted vinylic boronic acid derivatives and hydrogenation of the intermediately formed vinylic derivatives followed by the deprotection of only one PG- group with e.g. trifluoroacetic acid or other methods well known to the person skilled in the art are useful for the preparation of alkyl or haloalkyl derivatives of compounds of the general formula (XLVIII).

The amines of the formula (XLVIII) can react with a bromide, chloride or sulfonates of the formula (XLI) with methods well known to the person skilled in the art to give compounds of formula (XLIX). Finally, a deprotection reaction well known to the person skilled in the art produces compounds of the formula (Ia).

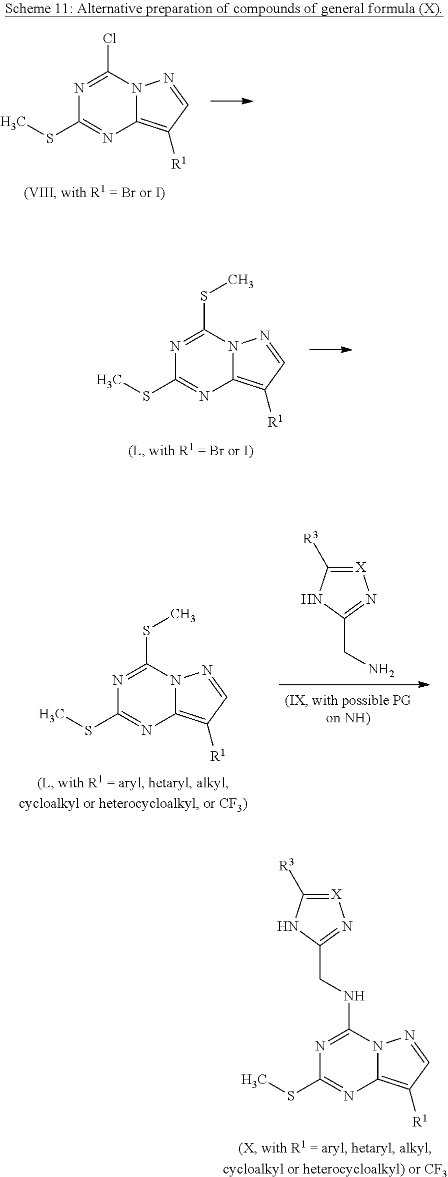

An alternative approach to compounds of the general formula (X) is depicted in scheme 11. Starting from chloride (VIII) the reaction with sodium methanthiolate gives compounds of the general formula (L). The bromine or iodine atom can be substituted as mentioned in many schemes before, e.g. by a Suzuki reaction using a corresponding boronic acid derivative in the presence of a Pd-catalyst and a base in an appropriate solvent or a trifluormethylation reaction using methyl difluoro(fluorosulfonyl)acetate in the presence of copper(I)iodide in an appropriate solvent or other trifluoromethylating reagents, e.g. trimethyl(trifluoromethyl)silane in the presence of potassium fluoride and copper(I)iodide in an inert solvent such as 1-methyl-pyrrolidin-2-one (e.g. WO2014/99836 or Journal of Fluorine Chemistry (2013), 156, 170-176) or triethyl(trifluoromethyl)silane in the presence of potassium fluoride and copper(I)iodide in an inert solvent (e.g. WO2004/022560) or trifluoromethylsulfonium salt (e.g. Angewandte Chemie, International Edition (2011), 50(8), 1896-1900) or another method described before to give compounds of general formula (L). These compounds then react via a nucleophilic substitution using amines such as (IX) to produce intermediate (X) which is a precursor of compounds (1).

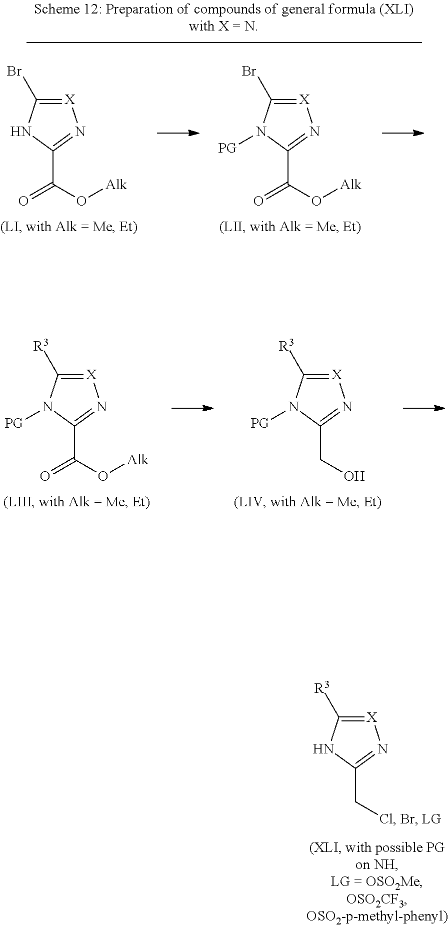

Compounds of the general formula XLI wit X═N can be assembled starting from commercially available triazole carboxylic ester (LI). Protection of the NH with e.g. tetrahydropyranyl, p-methoxy-benzyl or other protecting groups well known to the person skilled in the art gives compounds (LII) which then can be modified via metallo-organic reaction such as Suzuki reaction or others well known to the person skilled in the art to obtain compounds (LIII). The reduction of the ester in (LIII) using reducing agents such as lithium borohydride, lithium aluminum hydride or diisobutylaluminium hydride gives the alcohols (LIV) which then can be transformed to chlorides, bromides or sulfonates using methodologies well known to the person skilled in the art. If necessary a final deprotection will give the compounds with the free NH- group.

The present invention includes the intermediate compounds which are disclosed in the Example Section of this text, infra.

The compounds of general formula (I) of the present invention can be converted to any salt, preferably pharmaceutically acceptable salts, by any method which is known to the person skilled in the art. Similarly, any salt of a compound of general formula (I) of the present invention can be converted into the free compound, by any method which is known to the person skilled in the art.

Compounds of general formula (I) of the present invention demonstrate a valuable pharmacological spectrum of action which could not have been predicted. The compounds of the present invention effectively inhibit the activity of CDK12 for which data are given in the biological experimental section and may therefore be used for the treatment and/or prophylaxis of hyperproliferative disorders, such as cancer disorders in humans and animals.

### Methods and Administration

Compounds of general formula (I) of the present invention demonstrate a valuable pharmacological spectrum of action and pharmacokinetic profile, both of which could not have been predicted. Compounds of the present invention have surprisingly been found to effectively impair the activity of CDK12, showing a strong CDK12 degrading potency which induce the proteolytic degradation of CDK12 protein in the cell resulting in an increased selectivity against other kinases. Therefore, it is possible that said compounds can be used for the treatment and/or prophylaxis of diseases, preferably hyperproliferative disorders in humans and animals.

Further, CDK12 has been identified as a druggable target for addressing the RNA-based disease myotonic dystrophy type 1 (DM1) (Ketley et al., Sci. Transl. Med. 12, eaaz2415 (2020)). Thus, it is possible that compounds of general formula (I) of the present invention can be used for the treatment and/or prophylaxis of diseases in which CDK12 is involved, such as myotonic dystrophy type 1 (DM1).

As used herein, “prophylaxis” includes a use of the compound that, in a statistical sample, reduces the occurrence of the disorder or condition in the treated sample relative to an untreated control sample, or delays the onset or reduces the severity of one or more symptoms of the disorder or condition relative to the untreated control sample, when administered to prior to the onset of the disorder or condition.

Compounds of the present invention can be utilized to inhibit, block, reduce, decrease, etc., cell proliferation and/or cell division, and/or produce apoptosis, which are all types of “treatment”. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the disorder.

Hyperproliferative disorders include, but are not limited to, for example: psoriasis, keloids, and other hyperplasias affecting the skin, benign prostate hyperplasia (BPH), solid tumours, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukaemias.

Examples of breast cancers include, but are not limited to, invasive ductal carcinoma, invasive lobular carcinoma, ductal carcinoma in situ, and lobular carcinoma in situ.

Examples of cancers of the respiratory tract include, but are not limited to, small-cell and non-small-cell lung carcinoma, as well as bronchial adenoma and pleuropulmonary blastoma.

Examples of brain cancers include, but are not limited to, brain stem and hypothalamic glioma, cerebellar and cerebral astrocytoma, medulloblastoma, ependymoma, as well as neuroectodermal and pineal tumour.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the digestive tract include, but are not limited to, anal, colon, colorectal, oesophageal, gallbladder, gastric, pancreatic, rectal, small-intestine, and salivary gland cancers.

Tumours of the urinary tract include, but are not limited to, bladder, penile, kidney, renal pelvis, ureter, urethral and human papillary renal cancers.

Eye cancers include, but are not limited to, intraocular melanoma and retinoblastoma.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to, basal cell carcinoma, squamous cell carcinoma, Kaposi's sarcoma, malignant melanoma, Merkel cell skin cancer and non-melanoma skin cancer.

Head-and-neck cancers include, but are not limited to, laryngeal, hypopharyngeal, nasopharyngeal, oropharyngeal cancer, lip and oral cavity cancer and squamous cell.

Lymphomas include, but are not limited to, AIDS-related lymphoma, chronic lymphocytic lymphoma (CLL), non-Hodgkin's lymphoma (NHL), T-non-Hodgkin lymphoma (T-NHL), subtypes of NHL such as Diffuse Large Cell Lymphoma (DLBCL), activated B-cell DLBCL, germinal center B-cell lymphoma DLBCL, double-hit lymphoma and double-expressor lymphoma; anaplastic large cell lymphoma, B-cell lymphoma, cutaneous T-cell lymphoma, Burkitt's lymphoma, follicular lymphoma, hairy cell lymphoma, Hodgkin's disease, mantle cell lymphoma (MCL), lymphoma of the central nervous system, small lymphocytic lymphoma and chronic lymphocytic lymphoma and Sezary syndrome.

Sarcomas include, but are not limited to, sarcoma of the soft tissue, osteosarcoma, malignant fibrous histiocytoma, lymphosarcoma, and rhabdomyosarcoma.

Leukemias include, but are not limited to acute lymphoblastic leukemia, acute myeloid leukemia, (acute) T-cell leukemia, acute lymphoblastic leukemia, acute lymphocytic leukemia (ALL), acute monocytic leukemia (AML), acute promyelocytic leukemia (APL), bisphenotypic B myelomonocytic leukemia, chronic lymphocytic leukemia (CLL), chronic myelogenous leukemia, chronic myeloid leukemia (CML), chronic myelomonocytic leukemia (CMML), large granular lymphocytic leukemia, plasma cell leukemia and also myelodysplastic syndrome (MDS), which can develop into an acute myeloid leukemia.

The present invention also provides methods of treating angiogenic disorders including diseases associated with excessive and/or abnormal angiogenesis.

Inappropriate and ectopic expression of angiogenesis can be deleterious to an organism. A number of pathological conditions are associated with the growth of extraneous blood vessels. These include, for example, diabetic retinopathy, ischemic retinal-vein occlusion, and retinopathy of prematurity [Aiello et al., New Engl. J. Med., 1994, 331, 1480; Peer et al., Lab. Invest., 1995, 72, 638], age-related macular degeneration (AMD) [Lopez et al., Invest. Ophthalmol. Vis. Sci., 1996, 37, 855], neovascular glaucoma, psoriasis, retrolental fibroplasias, angiofibroma, inflammation, rheumatoid arthritis (RA), restenosis, in-stent restenosis, vascular graft restenosis, etc. In addition, the increased blood supply associated with cancerous and neoplastic tissue, encourages growth, leading to rapid tumour enlargement and metastasis. Moreover, the growth of new blood and lymph vessels in a tumour provides an escape route for renegade cells, encouraging metastasis and the consequence spread of the cancer. Thus, compounds of general formula (I) of the present invention can be utilized to treat and/or prevent any of the aforementioned angiogenesis disorders, for example by inhibiting and/or reducing blood vessel formation; by inhibiting, blocking, reducing, decreasing, etc. endothelial cell proliferation, or other types involved in angiogenesis, as well as causing cell death or apoptosis of such cell types.

These disorders have been well characterized in humans, but also exist with a similar etiology in other mammals, and can be treated by administering pharmaceutical compositions of the present invention.

The term “treating” or “treatment” as stated throughout this document is used conventionally, for example the management or care of a subject for the purpose of combating, alleviating, reducing, relieving and/or improving the condition of a disease or disorder, such as a carcinoma.

The compounds of the present invention can be used in particular in therapy and prevention, i.e. prophylaxis, of tumour growth and metastases, especially in solid tumours of all indications and stages with or without pre-treatment of the tumour growth.

Generally, the use of chemotherapeutic agents and/or anti-cancer agents in combination with a compound or pharmaceutical composition of the present invention will serve to:
1. yield better efficacy in reducing the growth of a tumour or even eliminate the tumour as compared to administration of either agent alone,
2. provide for the administration of lesser amounts of the administered chemo-therapeutic agents,
3. provide for a chemotherapeutic treatment that is well tolerated in the patient with fewer deleterious pharmacological complications than observed with single agent chemotherapies and certain other combined therapies,
4. provide for treating a broader spectrum of different cancer types in mammals, especially humans,
5. provide for a higher response rate among treated patients,
6. provide for a longer survival time among treated patients compared to standard chemotherapy treatments,
7. provide a longer time for tumour progression, and/or
8. yield efficacy and tolerability results at least as good as those of the agents used alone, compared to known instances where other cancer agent combinations produce antagonistic effects.

In addition, the compounds of general formula (I) of the present invention can also be used in combination with radiotherapy and/or surgical intervention.

In a further embodiment of the present invention, the compounds of general formula (I) of the present invention may be used to sensitize a cell to radiation, i.e. treatment of a cell with a compound of the present invention prior to radiation treatment of the cell renders the cell more susceptible to DNA damage and cell death than the cell would be in the absence of any treatment with a compound of the present invention. In one aspect, the cell is treated with at least one compound of general formula (I) of the present invention.

Thus, the present invention also provides a method of killing a cell, wherein a cell is administered one or more compounds of the present invention in combination with conventional radiation therapy.

The present invention also provides a method of rendering a cell more susceptible to cell death, wherein the cell is treated with one or more compounds of general formula (I) of the present invention prior to the treatment of the cell to cause or induce cell death. In one aspect, after the cell is treated with one or more compounds of general formula (I) of the present invention, the cell is treated with at least one compound, or at least one method, or a combination thereof, in order to cause DNA damage for the purpose of inhibiting the function of the cell or killing the cell.

In other embodiments of the present invention, a cell is killed by treating the cell with at least one DNA damaging agent, i.e. after treating a cell with one or more compounds of general formula (I) of the present invention to sensitize the cell to cell death, the cell is treated with at least one DNA damaging agent to kill the cell. DNA damaging agents useful in the present invention include, but are not limited to, chemotherapeutic agents (e.g. cis platin), ionizing radiation (X-rays, ultraviolet radiation), carcinogenic agents, and mutagenic agents.

In other embodiments, a cell is killed by treating the cell with at least one method to cause or induce DNA damage. Such methods include, but are not limited to, activation of a cell signalling pathway that results in DNA damage when the pathway is activated, inhibiting of a cell signalling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In some embodiments, a compound of general formula (I) of the present invention is administered to a cell prior to the radiation or other induction of DNA damage in the cell. In some embodiments of the invention, a compound of general formula (I) of the present invention is administered to a cell concomitantly with the radiation or other induction of DNA damage in the cell. In yet some embodiments of the invention, a compound of general formula (I) of the present invention is administered to a cell after radiation or other induction of DNA damage in the cell has begun. In yet some embodiments of the invention, a compound of general formula (I) of the present invention is administered to a cell immediately after radiation or other induction of DNA damage in the cell has begun. In some embodiments, the cell is in vitro. In another embodiment, the cell is in vivo.

Thus in some embodiments, the present invention includes a method of inhibiting proliferation of a cell and/or the induction of apoptosis in a cell, comprising contacting the cell with a compound of formula (I).

Another aspect of the invention is a method for treating, preventing or prophylaxing cancer (i.e. a method for the treatment, prevention or prophylaxis of cancer) in a subject (e.g., human, other mammal, such as rat, etc.) by administering an effective amount of at least one compound of general formula (I), or a pharmaceutically acceptable salt, polymorph, metabolite, hydrate, solvate or ester thereof to the subject.

In some embodiments, the subject may be administered a medicament, comprising at least one compound of general formula (I) and one or more pharmaceutically acceptable carriers, excipients and/or diluents.

Furthermore in some embodiments, the present invention includes a method of using a compound of general formula (I) for the treatment of diseases.

Particularly in some embodiments, the present invention includes a method of treating a hyperproliferative disease, more particularly cancer, comprising administering an effective amount of at least one compound of general formula (I) to a subject in need thereof.

Particularly in some embodiments, the present invention includes a method of treating a hyperproliferative disease, more particularly cancer, comprising administering an effective amount of at least one compound of general formula (I) having a ratio (IC50 CDK12 hATP)/(DC50 CDK12) which is equal or greater than 20 and/or a (DC50 CDK12) value which is equal or lower than 200 nM to a subject in need thereof.

In some embodiments, the method of treatment and/or prophylaxis of a hyperproliferative disorder in a subject may comprise administering to the subject an effective amount of a compound of general formula (I). The hyperproliferative disorder may be, for example, cancer (e.g., lung cancer, breast cancer, acute myeloid leukemia, lymphoma, glioblastoma, prostate cancer, etc.).

Furthermore in some embodiments, the present invention includes a method of treating cancer, particularly lymphoma, non-Hodgkin-lymphoma type, diffuse large B-cell lymphoma subtype, acute leukemia, acute myeloid leukemia type, multiple myeloma, ovarian cancer, comprising administering an effective amount of at least one compound of formula (I) to a subject in need thereof.

Furthermore in some embodiments, the present invention includes a method of treating cancer, particularly multiple myeloma, ovarian carcinoma, acute monocytic leukemia, melanoma and lung cancer, comprising administering an effective amount of at least one compound of formula (I) to a subject in need thereof.

Furthermore in some embodiments, the present invention includes a method of treating cancer, particularly breast cancer; lung cancer; lymphoma including non-Hodgkin-lymphoma type, diffuse large B-cell lymphoma subtype including GC-DLBCL* and ABC-DLBCL** subtypes, and mantle cell lymphoma; acute leukemia, acute myeloid leukemia type, acute monocytic leukemia; melanoma; multiple myeloma; ovarian cancer; and pancreas cancer, comprising administering an effective amount of at least one compound of formula (I) to a subject in need thereof according to any one of claims 1-9. GC-DLBCL means Germinal B-cell Diffuse Large B-Cell Lymphoma and ** ABC-DLBCL means Activated B-cell Diffuse Large B-Cell Lymphoma.

Furthermore in some embodiments, the present invention includes a method of treating cancer, particularly breast cancer, lung cancer, diffuse large B-cell lymphoma subtype including GC-DLBCL* and ABC-DLBCL** subtypes, mantle cell lymphoma, acute monocytic leukemia, melanoma, ovarian cancer, and pancreas cancer comprising administering an effective amount of at least one compound of formula (I) to a subject in need thereof according to any one of claims 1-9. Furthermore in some embodiments, the present invention provides a compound of formula (I) for use of treating diseases. Furthermore in some embodiments, the present invention includes a method of treating cancer, particularly breast cancer; lymphoma, leukemia, multiple myeloma; and ovarian cancer, comprising administering an effective amount of at least one compound of formula (I) to a subject in need thereof.

Furthermore in some embodiments, the present invention includes a method of treating cancer, particularly lymphoma, non-Hodgkin-lymphoma type, diffuse large B-cell lymphoma subtype, acute leukemia, acute myeloid leukemia type, multiple myeloma, and ovarian cancer, comprising administering an effective amount of at least one compound of formula (I) to a subject in need thereof.

Furthermore in some embodiments, the present invention includes a method of treating cancer, particularly breast cancer, lymphoma (including non-Hodgkin-lymphoma type, diffuse large B-cell lymphoma subtype, mantle cell lymphoma), leukemia (including acute monocytic leukemia), liver cancer, multiple myeloma, melanoma, non-small cell lung cancer, small cell lung cancer, ovarian cancer, ovarian carcinoma, stomach cancer, and squamous cell carcinoma, comprising administering an effective amount of at least one compound of formula (I) to a subject in need thereof.

Furthermore in some embodiments, the present invention includes a method of treating cancer, particularly breast cancer, diffuse large B-cell lymphoma subtype, mantle cell lymphoma, acute monocytic leukemia, liver cancer, multiple myeloma, melanoma, non-small cell lung cancer, small cell lung cancer, ovarian cancer, ovarian carcinoma, prostate cancer, stomach cancer, and squamous cell carcinoma, comprising administering an effective amount of at least one compound of formula (I) to a subject in need thereof.

Furthermore in some embodiments, the present invention includes a method of treating cancer, particularly bladder cancer, bone cancer, brain cancer, breast cancer, colon cancer (colorectal cancer), endometrial (uterine) cancer, gastric cancer, head and neck cancer, kidney cancer, leukemia, liver cancer, lung cancer, lymphoma, lung cancer, myeloma, neuroblastoma, ovarian cancer, pancreatic cancer, rhabdoid tumor, sarcoma and skin cancer, comprising administering an effective amount of at least one compound of formula (I) to a subject in need thereof.

Furthermore in some embodiments, the present invention includes a method of treating cancer, particularly breast cancer, liver cancer, lung cancer, ovarian cancer, endometrial cancer, cervical cancer, colorectal cancer, gastric cancer, esophageal cancer, bladder cancer, prostate cancer, sarcoma, glioblastoma and acute myeloid leukemia comprising administering an effective amount of at least one compound of formula (I) to a subject in need thereof.

Furthermore in some embodiments, the present invention includes a method of treating cancer, particularly lung cancer, breast cancer, liver cancer, colorectal cancer, gastric cancer, prostate cancer and leukemia comprising administering an effective amount of at least one compound of formula (I) to a subject in need thereof.

Furthermore in some embodiments, the present invention includes a method of treating myotonic dystrophy type 1 (DM1) comprising administering an effective amount of at least one compound of general formula (I) to a subject in need thereof.

In accordance with some embodiments, the present invention provides compounds of general formula (I), as described supra, or stereoisomers, tautomers, N-oxides, hydrates, solvates, and salts thereof, particularly pharmaceutically acceptable salts thereof, or mixtures of same, for use in the treatment and/or prophylaxis of diseases, in particular hyperproliferative disorders.

In accordance with some embodiments, the present invention provides compounds of general formula (I) having a ratio (IC50 CDK12 hATP)/(DC50 CDK12) which is equal or greater than 20 and/or a (DC50 CDK12) value which is equal or lower than 200 nM, as described supra, or stereoisomers, tautomers, N-oxides, hydrates, solvates, and salts thereof, particularly pharmaceutically acceptable salts thereof, or mixtures of same, for use in the treatment and/or prophylaxis of diseases, in particular hyperproliferative disorders.

Furthermore in accordance with a further aspect, the present invention provides a compound of formula (I) for use of treating diseases. Furthermore in accordance with a further aspect, the present invention provides a compound of formula (I) having a ratio (IC50 CDK12 hATP)/(DC50 CDK12) which is equal or greater than 20 and/or a (DC50 CDK12) value which is equal or lower than 200 nM for use of treating diseases.

In in accordance with a further aspect, the present invention includes a compound of general formula (I) for use in a method of inhibiting proliferation of a cell and/or the induction of apoptosis in a cell, comprising contacting the cell with a compound of formula (I).

Particularly in some embodiments, the present invention includes compounds of general formula (I) for use in a method of treating a hyperproliferative disease, more particularly wherein the hyperproliferative disease is cancer, and yet even more particularly wherein the cancer disease is selected from lymphoma, non-Hodgkin-lymphoma type, diffuse large B-cell lymphoma subtype, ovarian cancer, multiple myeloma, acute leukemia, and acute myeloid leukemia.

More particularly in some embodiments, the present invention includes compounds of general formula (I) for use in a method of treating a hyperproliferative disease, more particularly wherein the hyperproliferative disease is cancer, and yet even more particularly wherein the cancer disease is selected from breast cancer; lymphoma, leukemia, multiple myeloma; and ovarian cancer.

Particularly in some embodiments, the present invention includes compounds of general formula (I) for use in a method of treating a hyperproliferative disease, more particularly wherein the hyperproliferative disease is cancer, and yet even more particularly wherein the cancer is selected from breast cancer; esophageal cancer; liver cancer; lung cancer; lymphoma including non-Hodgkin-lymphoma type, diffuse large B-cell lymphoma subtype including GC-DLBCL* and ABC-DLBCL** subtypes, and mantle cell lymphoma; acute leukemia, acute myeloid leukemia type, acute monocytic leukemia; melanoma; multiple myeloma; melanoma; ovarian cancer; or pancreas cancer.

More particularly in some embodiments, the present invention includes compounds of general formula (I) for use in a method of treating cancer wherein the cancer disease is selected from breast cancer; lymphoma, leukemia, multiple myeloma; and ovarian cancer.

More particularly in some embodiments, the present invention includes compounds of general formula (I) for use in a method of treating cancer wherein the cancer disease is selected from breast cancer, liver cancer, lung cancer, ovarian cancer, endometrial cancer, cervical cancer, colorectal cancer, gastric cancer, esophageal cancer, bladder cancer, prostate cancer, sarcoma, glioblastoma, and acute myeloid leukemia.

More particularly in some embodiments, the present invention includes compounds of general formula (I) for use in a method of treating cancer wherein the cancer disease is selected from lung cancer, breast cancer, liver cancer, colorectal cancer, gastric cancer, prostate cancer, and leukemia.

Furthermore in some embodiments, the present invention includes compounds of general formula (I) for use in a method of treating myotonic dystrophy type 1 (DM1).

In some embodiments, the present invention includes the use of the compounds of general formula (I) for the manufacture of a medicament for the treatment and/or prophylaxis of a hyperproliferative disease.

In some embodiments, the present invention includes the use of the compounds of general formula (I) for the manufacture of a medicament for the treatment and/or prophylaxis of a hyperproliferative disease, wherein the hyperproliferative disease is cancer.

In some embodiments, the present invention includes the use of the compounds of general formula (I) having a ratio (IC50 CDK12 hATP)/(DC50 CDK12) which is equal or greater than 20 and/or a (DC50 CDK12) value which is equal or lower than 200 nM for the manufacture of a medicament for the treatment and/or prophylaxis of a hyperproliferative disease.

In some embodiments, the present invention includes the use of the compounds of general formula (I) having a ratio (IC50 CDK12 hATP)/(DC50 CDK12) which is equal or greater than 20 and/or a (DC50 CDK12) value which is equal or lower than 200 nM for the manufacture of a medicament for the treatment and/or prophylaxis of a hyperproliferative disease, wherein the hyperproliferative disease is cancer. In some embodiments, the present invention includes the use of the compounds of general formula (I) for the manufacture of a medicament for the treatment of a hyperproliferative disease, particularly cancer and more particularly lymphoma, non-Hodgkin-lymphoma type, diffuse large B-cell lymphoma subtype, ovarian cancer, multiple myeloma, acute leukemia, and acute myeloid leukemia type.

In some embodiments, the present invention includes the use of the compounds of general formula (I) for the manufacture of a medicament for the treatment of a hyperproliferative disease, particularly cancer and more particularly breast cancer, liver cancer, lung cancer, ovarian cancer, endometrial cancer, cervical cancer, colorectal cancer, gastric cancer, esophageal cancer, bladder cancer, prostate cancer, sarcoma, glioblastoma, and acute myeloid leukemia.

In some embodiments, the present invention includes the use of the compounds of general formula (I) for the manufacture of a medicament for the treatment of a hyperproliferative disease, particularly cancer and more particularly lung cancer, breast cancer, liver cancer, colorectal cancer, gastric cancer, prostate cancer, and leukemia.

In some embodiments, the present invention provides use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer.

In some embodiments, the present invention provides use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer, more particularly breast cancer, liver cancer, lung cancer, ovarian cancer, endometrial cancer, cervical cancer, colorectal cancer, gastric cancer, esophageal cancer, bladder cancer, prostate cancer, sarcoma, glioblastoma, and acute myeloid leukemia.

Furthermore in some embodiments, the present invention includes the use of the compounds of general formula (I) for the manufacture of a medicament for the treatment of myotonic dystrophy type 1 (DM1).

In some embodiments, the present invention provides a method of treatment and/or prophylaxis of diseases, in particular hyperproliferative disorders, particularly cancer, comprising administering an effective amount of a compound of general formula (I), as described supra, or stereoisomers, tautomers, N-oxides, hydrates, solvates, and salts thereof, particularly pharmaceutically acceptable salts thereof, or mixtures of same to a subject in need thereof.

In some embodiments, the present invention provides a method of treatment and/or prophylaxis of diseases, in particular hyperproliferative disorders, particularly cancer, more particularly breast cancer, liver cancer, lung cancer, ovarian cancer, endometrial cancer, cervical cancer, colorectal cancer, gastric cancer, esophageal cancer, bladder cancer, prostate cancer, sarcoma, glioblastoma, and acute myeloid leukemia comprising administering an effective amount of a compound of general formula (I), as described supra, or stereoisomers, tautomers, N-oxides, hydrates, solvates, and salts thereof, particularly pharmaceutically acceptable salts thereof, or mixtures of same to a subject in need thereof.

Furthermore in some embodiments, the present invention provides a method of treatment of myotonic dystrophy type 1 (DM1) comprising administering an effective amount of a compound of general formula (I), as described supra, or stereoisomers, tautomers, N-oxides, hydrates, solvates, and salts thereof, particularly pharmaceutically acceptable salts thereof, or mixtures of same to a subject in need thereof.

In some embodiments, the present invention provides pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore provides pharmaceutical compositions, in particular medicaments, which comprise at least one compound according to the invention, conventionally together with one or more pharmaceutically suitable excipients, and to their use for the above mentioned purposes.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent. For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

In some embodiments, the present invention provides pharmaceutical combinations, in particular medicaments, comprising at least one compound of general formula (I) of the present invention and at least one or more further active ingredients, in particular for the treatment and/or prophylaxis of a hyperproliferative disorder, particularly cancer. Particularly, the present invention provides a pharmaceutical combination, which comprises:
one or more first active ingredients, in particular compounds of general formula (I) as defined supra, and
one or more further active ingredients, in particular for the treatment and/or prophylaxis of a hyperproliferative disorder, particularly cancer.

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also provides such pharmaceutical combinations. For example, the compounds of the present invention can be combined with known anti-cancer agents.

Examples of anti-cancer agents include:

131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, altretamine, amifostine, aminoglutethimide, hexyl aminolevulinate, amrubicin, amsacrine, anastrozole, ancestim, anethole dithiolethione, anetumab ravtansine, angiotensin 1l, antithrombin Ill, apalutamide, aprepitant, arcitumomab, arglabin, arsenic trioxide, asparaginase, atezolizumab, avelumab, axicabtagene ciloleucel, axitinib, azacitidine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, lanreotide, lansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, lonidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid and zorubicin.

Based upon standard laboratory techniques known to evaluate compounds useful for the treatment of hyperproliferative disorders, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, it is possible for “drug holidays”, in which a patient is not dosed with a drug for a certain period of time, to be beneficial to the overall balance between pharmacological effect and tolerability. It is possible for a unit dosage to contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### EXPERIMENTAL SECTION

### Experimental Section—NMR Spectra

To the extent NMR peak forms and multiplicities are specified, they are stated as they appear in the spectra, possible higher order effects have not been considered.

The ¹H-NMR data of selected examples are listed in the form of ¹H-NMR peaklists. For each signal peak the δ value in ppm is given, followed by the signal intensity, reported in round brackets. The δ value-signal intensity pairs from different peaks are separated by commas. Therefore, a peaklist is described by the general form: δ₁ (intensity₁), δ₂ (intensity₂), . . . , δᵢ (intensityᵢ), . . . , δₙ (intensityₙ).

The intensity of a sharp signal correlates with the height (in cm) of the signal in a printed NMR spectrum. When compared with other signals, this data can be correlated to the real ratios of the signal intensities. In the case of broad signals, more than one peak, or the center of the signal along with their relative intensity, compared to the most intense signal displayed in the spectrum, are shown. A ¹H-NMR peaklist is similar to a classical ¹H-NMR readout, and thus usually contains all the peaks listed in a classical NMR interpretation. Moreover, similar to classical ¹H-NMR printouts, peaklists can show solvent signals, signals derived from stereoisomers of target compounds (also the subject of the invention), and/or peaks of impurities. The peaks of stereoisomers, and/or peaks of impurities are typically displayed with a lower intensity compared to the peaks of the target compounds (e.g., with a purity of >90%). Such stereoisomers and/or impurities may be typical for the particular manufacturing process, and therefore their peaks may help to identify the reproduction of our manufacturing process on the basis of “by-product fingerprints”. An expert who calculates the peaks of the target compounds by known methods (MestReC, ACD simulation, or by use of empirically evaluated expectation values), can isolate the peaks of target compounds as required, optionally using additional intensity filters. Such an operation would be similar to peak-picking in classical ¹H-NMR interpretation. A detailed description of the reporting of NMR data in the form of peaklists can be found in the publication “Citation of NMR Peaklist Data within Patent Applications” (cf. Research Disclosure Database Number 605005, 2014, 1 Aug. 2014, or http://www.researchdisclosure.com/searching-disclosures). In the peak picking routine, as described in the Research Disclosure Database Number 605005, the parameter “MinimumHeight” can be adjusted between 1% and 4%. Depending on the chemical structure and/or depending on the concentration of the measured compound it may be reasonable to set the parameter “MinimumHeight”<1%.

### Experimental Section—Abbreviations

The following table lists the abbreviations used in this paragraph and in the Intermediates and Examples section as far as they are not explained within the text body. Other abbreviations have their meanings customary per se to the skilled person. A comprehensive list of the abbreviations utilized by organic chemists of ordinary skill in the art appears presented in the first issue of each volume of the Journal of Organic Chemistry; this list is typically presented in a table titled “Standard List of Abbreviations”. In case of doubt, the abbreviations and/or their meaning according to the following table shall prevail.

TABLE 1
Abbreviations

| Abbreviation | Meaning |
|---|---|
| DMF | N,N-Dimethylformamide |
| DMSO | dimethylsulfoxide |
| ESI | electrospray (ES) ionisation |
| h, hr (hrs) | hour(s) |
| HPLC | high performance liquid chromatography |
| LC-MS | liquid chromatography-mass spectrometry |
| m | multiplet (NMR) |
| Min | minute(s) |
| MS | mass spectrometry |
| NMR | nuclear magnetic resonance spectroscopy: chemical |
| | shifts (δ) are given in ppm. The chemical shifts were |
| | corrected by setting the DMSO signal to 2.50 ppm using |
| | dmso-d6 unless otherwise stated. |
| rt, RT | room temperature |
| Rₜ, Rt | retention time |
| THF | tetrahydrofuran |
| UPLC | ultra performance liquid chromatography |
| UV | ultraviolet |
| δ | chemical shift |

Other abbreviations have their meanings customary per se to the skilled person.

The various aspects of the invention described in this application are illustrated by the following examples which are not meant to limit the invention in any way.

The example testing experiments described herein serve to illustrate the present invention and the invention is not limited to the examples given.

### Experimental Section—General Part

All reagents, for which the synthesis is not described in the experimental part, are either commercially available, or are known compounds or may be formed from known compounds by known methods by a person skilled in the art. Reactions were set up and started, e.g. by the addition of reagents, at temperatures as specified in the protocols; if no temperature is specified, the respective working step was performed at ambient temperature, i.e. between 18 and 25° C.

“Silicone filter” or “water resistant filter” refers to filter papers which are made hydrophobic (impermeable to water) by impregnation with a silicone. With the aid of these filters, water can be separated from water-immiscible organic solvents by means of a filtration (i.e. filter paper type MN 617 WA, Macherey-Nagel).

The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be removed by trituration using a suitable solvent or solvent mixture. In some cases, the compounds may be purified by chromatography, particularly flash column chromatography, using for example prepacked silica gel cartridges, e.g. Biotage SNAP cartridges KP-Sil® or KP-NH® in combination with a Biotage autopurifier system (SP4© or Isolera Four®) and eluents such as gradients of hexane/ethyl acetate or dichloromethane/ethanol. In flash column chromatography, unmodified (“regular”) silica gel may be used as well as aminophase functionalized silica gel. As used herein, “Biotage SNAP cartridge silica” refers to the use of regular silica gel; “Biotage SNAP cartridge NH₂ silica” refers to the use of aminophase functionalized silica gel. If reference is made to flash column chromatography or to flash chromatography in the experimental section without specification of a stationary phase, regular silica gel was used.

In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid, diethylamine or aqueous ammonia.

In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc.) of a compound of the present invention as isolated and as described herein is not necessarily the only form in which said compound can be applied to a biological assay in order to quantify the specific biological activity.

### UPLC-MS Standard Procedures

Analytical UPLC-MS was performed as described below. The masses (m/z) are reported from the positive mode electrospray ionisation unless the negative mode is indicated (ESI−).

Analytical UPLC methods:

### Method 1:

Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 mL/min; temperature: 60° C.; DAD scan: 210-400 nm.

### Method 2:

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 mL/min; temperature: 60° C.; DAD scan: 210-400 nm.

### Method 3:

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 mL/min; temperature: 60° C.; DAD scan: 210-400 nm.

### Method C:5-95AB, Shimadzu

Instrument: SHIMADZU LCMS-2020 SingleQuad; Column: Chromolith@Flash RP-18E 25-2 MM; eluent A: water+0.0375 vol % trifluoroacetic acid, eluent B: acetonitrile+0.01875 vol % trifluoroacetic acid; gradient: 0-0.8 min, 5-95% B, 0.8-1.2 min 95% B; flow 1.5 mL/min; temperature: 50° C.; PDA: 220 nm & 254 nm.

### Method D:5-95AB, Agilent

Instrument: Agilent 1100†G1956A SingleQuad; Column: Kinetex@ 5 μm EVO C18 30*2.1 mm; eluent A: water+0.0375 vol % trifluoroacetic acid, eluent B: acetonitrile+0.01875 vol % trifluoroacetic acid; gradient: 0-0.8 min 5-95% B, 0.8-1.2 min 95% B; flow 1.5 mL/min; temperature: 50° C.; PDA: 220 nm & 254 nm.

### Preparative HPLC Methods:

### Method HT Acidic:

Instrument: Waters Autopurificationsystem; Column: Waters XBrigde C18 5μ 100×30 mm; eluent A: water+0.1 vol % formic acid (99%), eluent B: acetonitrile; gradient; DAD scan: 210-400 nm.

### Method HT Basic:

Instrument: Waters Autopurificationsystem; Colum: Waters XBrigde C18 5μ 100×30 mm; eluent A: water+0.2 vol % aqueous ammonia (32%), eluent B: acetonitrile; gradient; DAD scan: 210-400 nm.

### Specific Optial Rotation Methods:

Method O1: Instrument: JASCO P2000 Polarimeter; wavelength 589 nm; temperature: 20° C.; integration time 10 s; path length 100 mm.

### Intermediate 1

### N-[(1H-benzimidazol-2-yl)methyl]-2-(methylsulfanyl)-8-(propan-2-yl)pyrazolo[1,5-a][1,3,5]triazin-4-amine

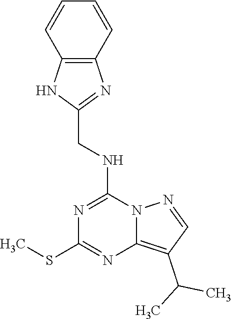 [SMILES: CSc1nc(NCc2nc3ccccc3[nH]2)n2ncc(C(C)C)c2n1]

4-chloro-2-(methylsulfanyl)-8-(propan-2-yl)pyrazolo[1,5-a][1,3,5]triazine (728 mg, 3.00 mmol, CAS 1453186-96-3, see European Journal of Medicinal Chemistry, 158, 1-6; 2018) and 1-(1H-benzimidazol-2-yl)methanamine hydrochloric acid salt) (826 mg, 4.50 mmol, CAS 5805-57-2) were dissolved in acetonitrile (10 mL), N,N-diisopropylethylamine (2.5 mL, 14 mmol) was added and the mixture was stirred overnight at 50° C. The mixture was evaporated, diluted with ethyl acetate, washed with sodium hydroxide (2M) and brine. The organic layer was dried and evaporated. The residue was stirred with methanol and the resulting precipitate was collected by filtration and dried to give 630 mg (99% purity, 59% yield) of the title compound, which was used without further purification.

LC-MS (Method 2): Rₜ=1.22 min; MS (ESIpos): m/z=354 [M+H]⁺

¹H-NMR (400 MHz, DMSO-d6) δ[ppm]: 1.298 (16.00), 1.316 (15.51), 2.075 (3.53), 2.408 (15.66), 2.518 (1.81), 2.523 (1.31), 3.061 (0.93), 3.078 (1.26), 3.095 (0.89), 4.902 (5.63), 5.759 (0.47), 7.122 (2.54), 7.129 (2.32), 7.136 (2.39), 7.145 (2.87), 7.155 (0.47), 7.471 (0.87), 8.030 (5.51).

### Intermediate 2

### N-[(1H-benzimidazol-2-yl)methyl]-2-(methanesulfonyl)-8-(propan-2-yl)pyrazolo[1,5-a][1,3,5]triazin-4-amine

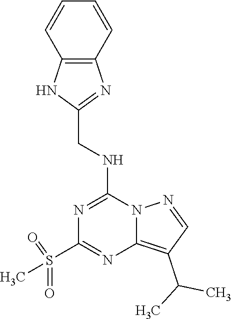 [SMILES: CC(C)c1cnn2c(NCc3nc4ccccc4[nH]3)nc(S(C)(=O)=O)nc12]

N-[(1H-benzimidazol-2-yl)methyl]-2-(methylsulfanyl)-8-(propan-2-yl)pyrazolo[1,5-a][1,3,5]triazin-4-amine (Intermediate 1, 3.04 g, 8.60 mmol) was dissolved in dichloromethane (57 mL), cooled to 0° C., mCPBA (6.36 g, 70% purity, 25.8 mmol) was added and the mixture was stirred for 3 h at rt. The mixture was diluted with saturated sodium bicarbonate solution and the layers were separated. The aqueous layer was extracted with dichloromethane and the combined organic layers were dried and evaporated. The residue was purified by flash chromatography (dichloromethane/ethanol gradient 0-10% ethanol) to give 1.96 g (99% purity, 59% yield) and 1.08 g (50% purity, 16% yield) the title compound.

LC-MS (Method 1): Rₜ=0.85 min; MS (ESIpos): m/z=386 [M+H]⁺

¹H-NMR (500 MHz, DMSO-d6) δ[ppm]: 0.902 (0.29), 0.954 (0.17), 0.966 (0.17), 1.040 (0.31), 1.053 (0.59), 1.067 (0.26), 1.334 (15.85), 1.348 (16.00), 1.664 (0.20), 2.064 (0.39), 2.514 (0.47), 2.518 (0.45), 2.522 (0.35), 3.160 (0.37), 3.174 (0.94), 3.187 (1.25), 3.201 (0.90), 3.215 (0.35), 3.269 (0.22), 3.333 (3.12), 5.025 (2.13), 5.037 (2.14), 5.758 (11.08), 7.138 (1.06), 7.148 (1.13), 7.413 (0.33), 7.527 (0.24), 7.543 (0.52), 7.559 (0.38), 7.900 (0.18), 8.340 (5.93), 9.986 (0.42), 9.998 (0.95), 10.009 (0.42), 12.300 (0.60).

### Intermediate 3

### ethyl [(1H-pyrazol-5-yl)carbamothioyl]carbamate

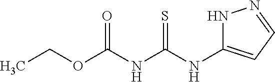

1H-pyrazol-5-amine (58.7 g, 706 mmol; CAS 1820-80-0) was dissolved in ethyl acetate (420 mL), under nitrogen, and stirred at 75° C. Ethyl carbonisothiocyanatidate (88 mL, 750 mmol; CAS 16182-04-0) was added dropwise at 75° C. and the mixture was stirred for 1 h at 75° C. The mixture was cooled to 0° C., filtered, washed with ethyl acetate and the solid was dried under reduced pressure at 50° C. to give 124 g (77% yield) of the title compound.

¹H-NMR (400 MHz, DMSO-d6) δ[ppm]: 1.232 (7.32), 1.250 (16.00), 1.267 (7.44), 2.518 (0.40), 4.184 (2.18), 4.201 (6.76), 4.219 (6.67), 4.237 (2.07), 5.889 (0.89), 5.893 (0.82), 6.998 (1.81), 7.003 (2.83), 7.008 (1.72), 7.697 (2.70), 7.867 (0.72), 7.872 (0.75), 11.317 (2.61), 12.036 (2.75), 12.709 (1.55).

### Intermediate 4

### 2-sulfanylpyrazolo[1,5-a][1,3,5]triazin-4-ol

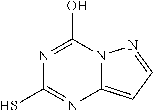 [SMILES: Oc1nc(S)nc2ccnn12]

Ethyl [(1H-pyrazol-5-yl)carbamothioyl]carbamate (Intermediate 3, 124 g, 580 mmol) was stirred in sodium hydroxide (550 mL, 2.0 M, 1.1 mol) for 3 h at rt. The mixture was cooled to 0° C. and sulfuric acid (580 mL, 2.0 M, 1.2 mol) was added dropwise. The suspension was filtered, washed with water and the solid was dried under reduced pressure at 50° C. to give 85.2 g (87% yield) of the title compound.

¹H-NMR (400 MHz, DMSO-d6) δ[ppm]: 2.518 (0.54), 3.349 (0.66), 5.888 (14.42), 5.892 (16.00), 7.866 (14.93), 7.870 (13.98), 12.730 (0.83), 13.450 (0.66).

### Intermediate 5

### 2-(methylsulfanyl)pyrazolo[1,5-a][1,3,5]triazin-4-ol

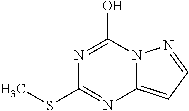

2-sulfanylpyrazolo[1,5-a][1,3,5]triazin-4-ol (Intermediate 4, 85.2 g, 507 mmol) was dissolved in ethanol (2.0 l) and sodium hydroxide (580 mL, 1.7 M, 1.0 mol). Iodomethane (32 mL, 510 mmol; CAS 74-88-4) was added dropwise at rt and the mixture was stirred for 2 h at rt. The mixture was cooled to 0° C., sulfuric acid (510 mL, 1.0 M, 510 mmol) was added dropwise and the mixture was stirred for 1 h at rt. The precipitate was collected by filtration, washed with water dried under reduced pressure at 50° C. The solid was stirred 2 times in acetonitrile, liquid phases were filtered off and the solid was washed with hexane and dried to give 60.5 g (65% yield) of the title compound.

¹H-NMR (400 MHz, DMSO-d6) δ[ppm]: 2.530 (16.00), 6.351 (3.35), 6.355 (3.08), 7.970 (2.67), 7.976 (3.22).

### Intermediate 6

### 8-bromo-2-(methylsulfanyl)pyrazolo[1,5-a][1,3,5]triazin-4-ol

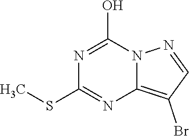

2-(methylsulfanyl)pyrazolo[1,5-a][1,3,5]triazin-4-ol (Intermediate 5, 59.0 g, 324 mmol) was dissolved in DMF (690 mL), cooled to 0° C., NBS (63.4 g, 356 mmol; CAS 128-08-5) dissolved in DMF (200 mL) was added dropwise and the mixture was stirred for 1 h at 0° C. The mixture was poured into water, stirred for 15 min, filtered and washed with water, acetonitrile and hexane. The solid was dried under reduced pressure at 50° C. to give 71.7 g (85% yield) of the title compound.

¹H-NMR (400 MHz, DMSO-d6) δ[ppm]: 8.113 (16.00).

### Intermediate 7

### 8-bromo-4-chloro-2-(methylsulfanyl)pyrazolo[1,5-a][1,3,5]triazine

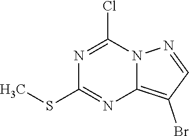

8-bromo-2-(methylsulfanyl)pyrazolo[1,5-a][1,3,5]triazin-4-ol (Intermediate 6, 33.3 g, 128 mmol) was dissolved in phosphorus oxychloride (170 mL, 1.8 mol; CAS 10025-87-3) and N,N-dimethylaniline (16 mL, 130 mmol; CAS 121-69-7) was added. The mixture was stirred for 3 h at 105° C. The mixture was poured carefully into ice water and neutralized with sodium bicarbonate. The suspension was filtered and washed with water and hexane to give 24.0 g (67% yield) of the title compound.

¹H-NMR (400 MHz, DMSO-d6) δ[ppm]: 2.518 (0.49), 2.567 (16.00), 8.116 (6.88).

### Intermediate 8

### N-[(1H-benzimidazol-2-yl)methyl]-8-bromo-2-(methylsulfanyl)pyrazolo[1,5-a][1,3,5]triazin-4-amine

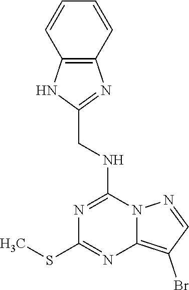 [SMILES: CSc1nc(NCc2nc3ccccc3[nH]2)n2ncc(Br)c2n1]

8-bromo-4-chloro-2-(methylsulfanyl)pyrazolo[1,5-a][1,3,5]triazine (Intermediate 7, 977 mg, 3.49 mmol) and 1-(1H-benzimidazol-2-yl)methanamine dihydrochloride (1.15 g, 5.24 mmol, CAS 5993-91-9) were dissolved in acetonitrile (11 mL), N,N-diisopropylethylamine (2.9 mL, 17 mmol) was added and the mixture was stirred overnight at 50° C. The mixture was evaporated, diluted with a mixture of dichloromethane and 2-propanol (4:1), washed with sodium hydroxide (2M) and brine. The organic layer was filtered. The solid was dried under reduced pressure to give 192 mg (95% purity, 13% yield) of the title compound, which was used without further purification.

LC-MS (Method 2): Rₜ=1.07 min; MS (ESIpos): m/z=390 [M+H]⁺

¹H-NMR (400 MHz, DMSO-d6) δ[ppm]: 2.433 (16.00), 2.518 (1.36), 2.522 (0.92), 4.930 (5.72), 7.129 (1.33), 7.137 (1.36), 7.145 (1.42), 7.152 (1.48), 8.283 (7.89).

### Intermediate 9

### N-[(1H-benzimidazol-2-yl)methyl]-8-bromo-2-(methanesulfonyl)pyrazolo[1,5-a][1,3,5]triazin-4-amine

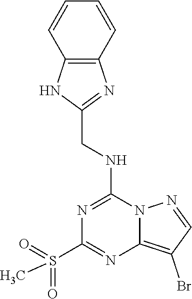 [SMILES: CS(=O)(=O)c1nc(NCc2nc3ccccc3[nH]2)n2ncc(Br)c2n1]

N-[(1H-benzimidazol-2-yl)methyl]-8-bromo-2-(methylsulfanyl)pyrazolo[1,5-a][1,3,5]triazin-4-amine (Intermediate 8, 650 mg, 1.67 mmol) was dissolved in dichloromethane (13 mL), cooled to 0° C., mCPBA (1.23 g, 70% purity, 5.00 mmol) was added and the mixture was stirred for 2 h at rt. The mixture was diluted with dichloromethane and washed with sat. sodium bicarbonate solution. The aqueous layer was extracted 2 times with a mixture of dichloromethane and 2-propanol and the combined organic layers were washed with water, dried and concentrated under reduced pressure to give 780 mg the title compound, which was used without further purification.

LC-MS (Method 2): Rₜ=0.67 min; MS (ESIpos): m/z=422 [M+H]⁺

### Intermediate 10

### N-[(1H-benzimidazol-2-yl)methyl]-2-(methylsulfanyl)pyrazolo[1,5-a][1,3,5]triazin-4-amine

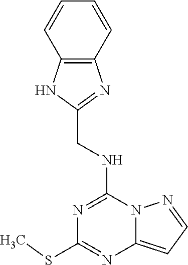 [SMILES: CSc1nc(NCc2nc3ccccc3[nH]2)n2nccc2n1]

4-chloro-2-(methylsulfanyl)pyrazolo[1,5-a][1,3,5]triazine (480 mg, 2.39 mmol, CAS 54346-19-9) and 1-(1H-benzimidazol-2-yl)methanamine dihydrochloride (790 mg, 3.59 mmol, CAS 5993-91-9) were dissolved in acetonitrile (7.7 mL), N,N-diisopropylethylamine (2.0 mL, 12 mmol) was added and the mixture was stirred overnight at 50° C. The mixture was diluted with a mixture of dichloromethane and 2-propanol (4:1) and washed with sodium hydroxide (2M). The precipitated from the organic layer was collected by filtration and tried to give 390 mg (52% yield) of the title compound, which was used without further purification.

LC-MS (Method 2): Rₜ=0.93 min; MS (ESIpos): m/z=312 [M+H]⁺

¹H-NMR (400 MHz, DMSO-d6) δ[ppm]: 2.406 (16.00), 2.518 (1.48), 2.522 (1.01), 4.917 (5.59), 6.335 (3.85), 6.340 (3.95), 7.126 (2.16), 7.135 (1.94), 7.142 (2.04), 7.149 (2.32), 7.159 (0.41), 7.480 (0.62), 8.126 (3.33), 8.131 (3.55).

### Intermediate 11

### N-[(1H-benzimidazol-2-yl)methyl]-8-chloro-2-(methylsulfanyl)pyrazolo[1,5-a][1,3,5]triazin-4-amine

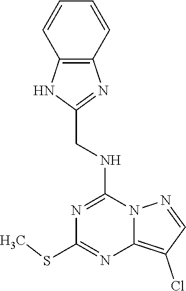 [SMILES: CSc1nc(NCc2nc3ccccc3[nH]2)n2ncc(Cl)c2n1]

N-[(1H-benzimidazol-2-yl)methyl]-2-(methylsulfanyl)pyrazolo[1,5-a][1,3,5]triazin-4-amine (Intermediate 10, 75.0 mg, 241 μmol) was dissolved in DMF (1.5 mL), 1-chloropyrrolidine-2,5-dione (38.6 mg, 289 μmol, CAS 128-09-6) was added and the mixture was stirred for 4 h at 60° C. The mixture was poured into water and extracted 3 times with ethyl acetate. The combined organic layers were dried and concentrated under reduced pressure to give 90 mg of the title compound, which was used in the next step without further purification.

LC-MS (Method 2): Rₜ=1.05 min; MS (ESIpos): m/z=346 [M+H]⁺

### Intermediate 12

### N-[(1H-benzimidazol-2-yl)methyl]-8-chloro-2-(methanesulfonyl)pyrazolo[1,5-a][1,3,5]triazin-4-amine

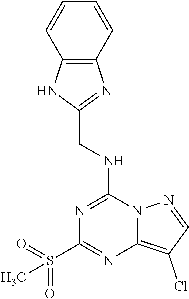 [SMILES: CS(=O)(=O)c1nc(NCc2nc3ccccc3[nH]2)n2ncc(Cl)c2n1]

N-[(1H-benzimidazol-2-yl)methyl]-8-chloro-2-(methylsulfanyl)pyrazolo[1,5-a][1,3,5]triazin-4-amine (Intermediate 11, 90.0 mg, 260 μmol) was dissolved in dichloromethane (1.7 mL), cooled to 0° C., mCPBA (192 mg, 70% purity, 781 μmol) was added and the mixture was stirred for 3 h at rt. The mixture was diluted with dichloromethane and washed with sat. sodium bicarbonate solution and brine. The organic layer was dried and concentrated under reduced pressure to give 105 mg of the title compound, which was used without further purification.

LC-MS (Method 2): Rₜ=0.65 min; MS (ESIpos): m/z=378 [M+H]⁺

### Intermediate 13

### 8-bromo-N-[(3H-imidazo[4,5-b]pyridin-2-yl)methyl]-2-(methylsulfanyl)pyrazolo[1,5-a][1,3,5]triazin-4-amine

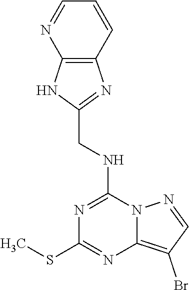 [SMILES: CSc1nc(NCc2nc3cccnc3[nH]2)n2ncc(Br)c2n1]

8-bromo-4-chloro-2-(methylsulfanyl)pyrazolo[1,5-a][1,3,5]triazine (Intermediate 7, 280 mg, 1.00 mmol) and 1-(3H-imidazo[4,5-b]pyridin-2-yl)methanamine dihydrochloride (332 mg, 1.50 mmol; CAS 914087-69-7) were dissolved in dry n-butanol (5.0 mL), N,N-diisopropylethylamine (870 μL, 5.0 mmol) was added and the mixture was stirred for 30 min at 90° C. in the microwave. The mixture was diluted with ethanol and water, The precipitate was collected by filtration, washed with water and ethanol and dried under reduced pressure to give 298 mg (73% yield) of the title compound.

LC-MS (Method 2): Rₜ=0.82 min; MS (ESIpos): m/z=391 [M+H]⁺

¹H-NMR (400 MHz, DMSO-d6) δ[ppm]: 2.416 (16.00), 2.518 (2.26), 2.523 (1.59), 4.957 (4.78), 7.176 (1.57), 7.188 (1.52), 7.195 (1.61), 7.207 (1.69), 8.285 (6.04).

### Intermediate 14

### 8-bromo-N-[(3H-imidazo[4,5-b]pyridin-2-yl)methyl]-2-(methanesulfonyl)pyrazolo[1,5-a][1,3,5]triazin-4-amine

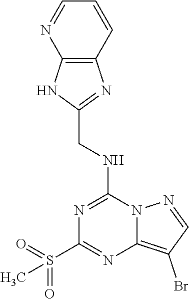 [SMILES: CS(=O)(=O)c1nc(NCc2nc3cccnc3[nH]2)n2ncc(Br)c2n1]

8-bromo-N-[(3H-imidazo[4,5-b]pyridin-2-yl)methyl]-2-(methylsulfanyl)pyrazolo[1,5-a][1,3,5]triazin-4-amine (Intermediate 13, 290 mg, 741 μmol) was dissolved in dichloromethane (5.7 mL), cooled to 0° C., mCPBA (548 mg, 70% purity, 2.22 mmol) was added and the mixture was stirred for 2 h at rt. The mixture was diluted with a mixture of dichloromethane and isopropanol and washed with sat. sodium bicarbonate solution. The organic layer was dried and concentrated under reduced pressure to give 165 mg (52% yield) the title compound, which was used without further purification.

LC-MS (Method 2): Rₜ=0.55 min; MS (ESIpos): m/z=423 [M+H]⁺

### Intermediate 15

### 8-bromo-N-[(7-methyl-3H-imidazo[4,5-b]pyridin-2-yl)methyl]-2-(methylsulfanyl)pyrazolo[1,5-a][1,3,5]triazin-4-amine

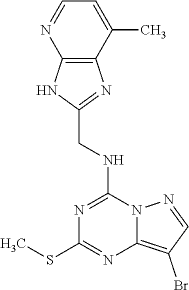 [SMILES: CSc1nc(NCc2nc3c(C)ccnc3[nH]2)n2ncc(Br)c2n1]

8-bromo-4-chloro-2-(methylsulfanyl)pyrazolo[1,5-a][1,3,5]triazine (Intermediate 7, 112 mg, 400 μmol) and 1-(7-methyl-3H-imidazo[4,5-b]pyridin-2-yl)methanamine dihydrochloride (141 mg, 600 μmol, CAS 1023813-52-6) were dissolved in dry n-butanol (2.0 mL), N,N-diisopropylethylamine (350 μL, 2.0 mmol) was added and the mixture was stirred for 30 min at 90° C. in the microwave. The mixture was diluted with ethanol and water, the precipitated was collected by filtration, washed with water and ethanol and dried under reduced pressure to give 168 mg (98% yield) of the title compound, which was used without further purification.

LC-MS (Method 2): Rₜ=0.92 min; MS (ESIpos): m/z=405 [M+H]⁺

### Intermediate 16

### 8-bromo-2-(methanesulfonyl)-N-[(7-methyl-3H-imidazo[4,5-b]pyridin-2-yl)methyl]pyrazolo[1,5-a][1,3,5]triazin-4-amine

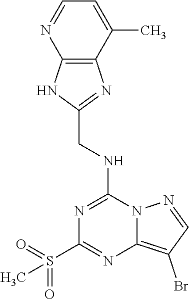 [SMILES: Cc1ccnc2[nH]c(CNc3nc(S(C)(=O)=O)nc4c(Br)cnn34)nc12]

8-bromo-N-[(7-methyl-3H-imidazo[4,5-b]pyridin-2-yl)methyl]-2-(methylsulfanyl)pyrazolo[1,5-a][1,3,5]triazin-4-amine (Intermediate 15, 160 mg, 395 μmol) was dissolved in dichloromethane (3.0 mL), cooled to 0° C., mCPBA (292 mg, 70% purity, 1.18 mmol) was added and the mixture was stirred for 2 h at rt. The mixture was diluted with a mixture of dichloromethane 2-propanol and washed with sat. sodium bicarbonate solution. The organic layer was dried and concentrated under reduced pressure to give 88 mg (51% yield) the title compound, which was used without further purification.

LC-MS (Method 1): Rₜ=0.68 min; MS (ESIpos): m/z=437 [M+H]⁺

### Intermediate 17

### 8-bromo-2-(methylsulfanyl)-N-[(4,5,6,7-tetrahydro-1H-benzimidazol-2-yl)methyl]pyrazolo[1,5-a][1,3,5]triazin-4-amine

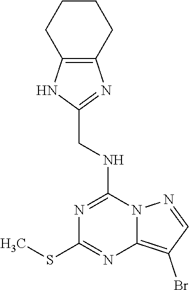 [SMILES: CSc1nc(NCc2nc3c([nH]2)CCCC3)n2ncc(Br)c2n1]

8-bromo-4-chloro-2-(methylsulfanyl)pyrazolo[1,5-a][1,3,5]triazine (Intermediate 7, 112 mg, 400 μmol) and 1-(4,5,6,7-tetrahydro-1H-benzimidazol-2-yl)methanamine-hydrogen chloride (1/2) (134 mg, 600 μmol) were dissolved in dry n-butanol (2.0 mL), N,N-diisopropylethylamine (350 μL, 2.0 mmol) was added and the mixture was stirred for 30 min at 90° C. in the microwave. The mixture was diluted with ethanol and water, filtered and washed with water and ethanol. The solid was dried under reduced pressure to give 75.0 mg (94% purity, 45% yield) of the title compound.

LC-MS (Method 2): Rₜ=1.12 min; MS (ESIpos): m/z=394 [M+H]⁺

¹H-NMR (400 MHz, DMSO-d6) δ[ppm]: 0.857 (1.02), 0.875 (0.51), 1.680 (7.88), 1.900 (0.90), 2.327 (1.44), 2.368 (2.01), 2.399 (2.58), 2.513 (7.16), 2.518 (4.49), 2.669 (1.35), 3.366 (0.42), 4.635 (9.71), 8.229 (16.00), 9.255 (0.60), 11.323 (1.02).

### Intermediate 18

### 8-bromo-2-(methanesulfonyl)-N-[(4,5,6,7-tetrahydro-1H-benzimidazol-2-yl)methyl]pyrazolo[1,5-a][1,3,5]triazin-4-amine

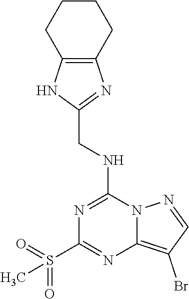 [SMILES: CS(=O)(=O)c1nc(NCc2nc3c([nH]2)CCCC3)n2ncc(Br)c2n1]

8-bromo-2-(methylsulfanyl)-N-[(4,5,6,7-tetrahydro-1H-benzimidazol-2-yl)methyl]pyrazolo[1,5-a][1,3,5]triazin-4-amine (Intermediate 17, 85.0 mg, 216 μmol) was dissolved in dichloromethane (1.7 mL), cooled to 0° C., mCPBA (159 mg, 70% purity, 647 μmol) was added and the mixture was stirred for 2 h at rt. The mixture was diluted with a mixture of dichloromethane and 2-propanol (4:1), washed with sat. sodium bicarbonate solution, dried and evaporated to give 82.0 mg (89% yield) of the title compound, which was used without further purification.

LC-MS (Method 2): Rₜ=0.80 min; MS (ESIpos): m/z=426 [M+H]⁺

### Intermediate 19

### 8-bromo-N-[(3H-imidazo[4,5-c]pyridin-2-yl)methyl]-2-(methylsulfanyl)pyrazolo[1,5-a][1,3,5]triazin-4-amine

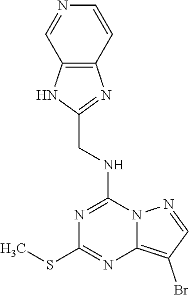 [SMILES: CSc1nc(NCc2nc3ccncc3[nH]2)n2ncc(Br)c2n1]

8-bromo-4-chloro-2-(methylsulfanyl)pyrazolo[1,5-a][1,3,5]triazine (Intermediate 7, 112 mg, 400 μmol) and 1-(3H-imidazo[4,5-c]pyridin-2-yl)methanamine dihydrochloride (133 mg, 600 μmol) were dissolved in dry n-butanol (2.0 mL), N,N-diisopropylethylamine (350 μL, 2.0 mmol) was added and the mixture was stirred for 30 min at 90° C. in the microwave. The mixture was evaporated and purified by preparative HPLC (HT basic) to give 108 mg (68% yield) of the title compound.

LC-MS (Method 2): Rₜ=0.77 min; MS (ESIpos): m/z=391 [M+H]⁺

¹H-NMR (400 MHz, DMSO-d6) δ[ppm]: 0.955 (0.81), 0.971 (0.76), 1.904 (0.78), 2.075 (7.06), 2.327 (1.39), 2.332 (1.00), 2.336 (0.42), 2.413 (16.00), 2.518 (4.76), 2.523 (3.40), 2.669 (1.44), 2.673 (1.00), 2.678 (0.42), 3.165 (1.76), 4.972 (6.11), 7.493 (0.49), 8.257 (3.35), 8.271 (3.10), 8.286 (1.05), 8.297 (8.35), 8.822 (1.15).

### Intermediate 20

### 8-bromo-N-[(3H-imidazo[4,5-c]pyridin-2-yl)methyl]-2-(methanesulfonyl)pyrazolo[1,5-a][1,3,5]triazin-4-amine

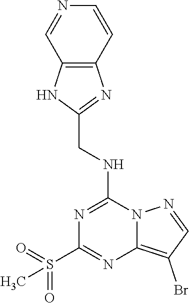 [SMILES: CS(=O)(=O)c1nc(NCc2nc3ccncc3[nH]2)n2ncc(Br)c2n1]

8-bromo-N-[(3H-imidazo[4,5-c]pyridin-2-yl)methyl]-2-(methylsulfanyl)pyrazolo[1,5-a][1,3,5]triazin-4-amine (Intermediate 19, 103 mg, 263 μmol) was dissolved in dichloromethane (2.0 mL), cooled to 0° C., mCPBA (195 mg, 70% purity, 790 μmol) was added and the mixture was stirred for 2 h at rt. The mixture was diluted with A mixture of dichloromethane and 2-propanol (4:1), washed with sat. sodium bicarbonate solution, dried and evaporated to give 85.0 mg (76% yield) of the title compound, which was used without further purification.

LC-MS (Method 2): Rₜ=0.52 min; MS (ESIpos): m/z=423 [M+H]⁺

### Intermediate 21

### 8-bromo-2-(methylsulfanyl)-N-[(5-phenyl-1H-imidazol-2-yl)methyl]pyrazolo[1,5-a][1,3,5]triazin-4-amine

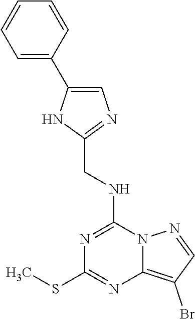 [SMILES: CSc1nc(NCc2ncc(-c3ccccc3)[nH]2)n2ncc(Br)c2n1]

8-bromo-4-chloro-2-(methylsulfanyl)pyrazolo[1,5-a][1,3,5]triazine (Intermediate 7, 140 mg, 500 μmol) and 1-(5-phenyl-1H-imidazol-2-yl)methanamine (173 mg, 1.00 mmol; CAS 175531-38-1) were dissolved in dry n-butanol (3.0 mL), N,N-diisopropylethylamine (440 μL, 2.5 mmol) was added and the mixture was stirred for 30 min at 90° C. in the microwave. The mixture was diluted with ethanol and water. The precipitate was collected by filtration, washed with water and ethanol and dried under reduced pressure to give 135 mg (64% yield) of the title compound.

LC-MS (Method 2): Rₜ=1.18 min; MS (ESIpos): m/z=416 [M+H]⁺

¹H-NMR (400 MHz, DMSO-d6) δ[ppm]: 2.481 (16.00), 2.518 (2.92), 2.523 (2.13), 4.772 (5.61), 7.159 (0.76), 7.177 (0.52), 7.300 (0.92), 7.319 (1.61), 7.337 (0.88), 7.541 (1.58), 7.725 (1.51), 7.744 (1.34), 8.253 (8.36), 11.940 (0.45).

### Intermediate 22

### 8-bromo-2-(methanesulfonyl)-N-[(5-phenyl-1H-imidazol-2-yl)methyl]pyrazolo[1,5-a][1,3,5]triazin-4-amine

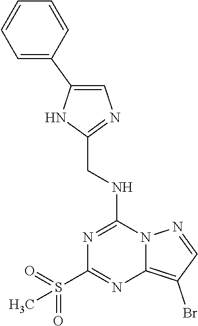 [SMILES: CS(=O)(=O)c1nc(NCc2ncc(-c3ccccc3)[nH]2)n2ncc(Br)c2n1]

8-bromo-2-(methylsulfanyl)-N-[(5-phenyl-1H-imidazol-2-yl)methyl]pyrazolo[1,5-a][1,3,5]triazin-4-amine (Intermediate 21, 135 mg, 324 μmol) was dissolved in dichloromethane (5.0 mL), cooled to 0° C., mCPBA (200 mg, 70% purity, 811 μmol) was added and the mixture was stirred for 1.5 h at rt. The mixture was diluted with dichloromethane, washed with sat. sodium bicarbonate solution, and the organic phase was dried and evaporated to give 130 mg (89% yield) of the title compound, which was used without further purification.

LC-MS (Method 2): Rₜ=0.79 min; MS (ESIneg): m/z=446 [M−H]⁻

### Intermediate 23

### 8-bromo-2-(methylsulfanyl)-N-[(5-phenyl-4H-1,2,4-triazol-3-yl)methyl]pyrazolo[1,5-a][1,3,5]triazin-4-amine

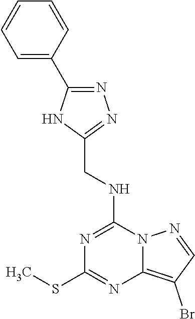 [SMILES: CSc1nc(NCc2nnc(-c3ccccc3)[nH]2)n2ncc(Br)c2n1]

8-bromo-4-chloro-2-(methylsulfanyl)pyrazolo[1,5-a][1,3,5]triazine (Intermediate 7, 706 mg, 2.52 mmol) and 1-(5-phenyl-4H-1,2,4-triazol-3-yl)methanamine dichydrochloride (749 mg, 3.03 mmol; CAS 1337882-06-0) were dissolved in n-butanol (15 mL), N,N-diisopropylethylamine (2.2 mL, 13 mmol) was added and the mixture was stirred for 2 h at 100° C. in the microwave. The mixture was diluted with dichloromethane and water, the layers were separated and the aqueous phase was washed 3 times with a mixture of dichloromethane and 2-propanol (4:1). The combined organic layers were dried and evaporated to give 1.23 g of the title compound, which was used without further purification.

LC-MS (Method 1): Rₜ=1.14 min; MS (ESIpos): m/z=417 [M+H]⁺

### Intermediate 24

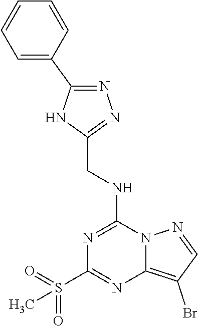 [SMILES: CS(=O)(=O)c1nc(NCc2nnc(-c3ccccc3)[nH]2)n2ncc(Br)c2n1]

8-bromo-2-(methylsulfanyl)-N-[(5-phenyl-4H-1,2,4-triazol-3-yl)methyl]pyrazolo[1,5-a][1,3,5]triazin-4-amine (Intermediate 23, 1.23 g, 2.95 mmol) was dissolved in acetonitrile (40 mL) and dichloromethane (20 mL), cooled to 0° C., mCPBA (1.82 g, 70% purity, 7.38 mmol) was added and the mixture was stirred for 16 h at rt. The mixture was diluted with A mixture of dichloromethane and 2-propanol (4:1), washed with sat. sodium bicarbonate solution, dried and evaporated to give 1.44 g of the title compound, which was used without further purification.

LC-MS (Method 1): Rₜ=0.93 min; MS (ESIpos): m/z=449 [M+H]⁺

### Intermediate 25

### 8-bromo-N-[(4,5-difluoro-1H-benzimidazol-2-yl)methyl]-2-(methylsulfanyl)pyrazolo[1,5-a][1,3,5]triazin-4-amine

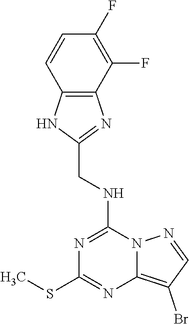 [SMILES: CSc1nc(NCc2nc3c(F)c(F)ccc3[nH]2)n2ncc(Br)c2n1]

8-bromo-4-chloro-2-(methylsulfanyl)pyrazolo[1,5-a][1,3,5]triazine (Intermediate 7, 280 mg, 1.00 mmol) and 1-(4,5-difluoro-1H-benzimidazol-2-yl)methanamine dihydrochloride (384 mg, 1.50 mmol, CAS 1201769-17-6) were dissolved in n-butanol (6.0 mL), N,N-diisopropylethylamine (870 μL, 5.0 mmol) was added and the mixture was stirred for 12 h at 90° C. The mixture was diluted with ethanol and water. The precipitate was collected by filtration, washed with water and ethanol and dried under reduced pressure to give 363 mg (85% yield) of the title compound.

LC-MS (Method 2): Rₜ=1.07 min; MS (ESIpos): m/z=426 [M+H]⁺

### Intermediate 26

### 8-bromo-N-[(4,5-difluoro-1H-benzimidazol-2-yl)methyl]-2-(methanesulfonyl)pyrazolo[1,5-a][1,3,5]triazin-4-amine

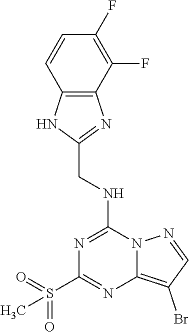 [SMILES: CS(=O)(=O)c1nc(NCc2nc3c(F)c(F)ccc3[nH]2)n2ncc(Br)c2n1]

8-bromo-N-[(4,5-difluoro-1H-benzimidazol-2-yl)methyl]-2-(methylsulfanyl)pyrazolo[1,5-a][1,3,5]triazin-4-amine (Intermediate 25, 258 mg, 605 μmol) was dissolved in dichloromethane (4.7 mL), cooled to 0° C., mCPBA (448 mg, 70% purity, 1.82 mmol) was added and the mixture was stirred for 2 h at rt. The mixture was diluted with dichloromethane, washed with sat. sodium bicarbonate solution, dried and evaporated to give 278 mg (100% yield) of the title compound, which was used without further purification.

LC-MS (Method 2): Rₜ=0.69 min; MS (ESIpos): m/z=460 [M+H]⁺

### Intermediate 27

### 8-bromo-N-{[4-(4-methylphenyl)-1H-imidazol-2-yl]methyl}-2-(methylsulfanyl)pyrazolo[1,5-a][1,3,5]triazin-4-amine

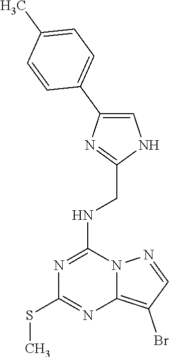 [SMILES: CSc1nc(NCc2nc(-c3ccc(C)cc3)c[nH]2)n2ncc(Br)c2n1]

8-bromo-4-chloro-2-(methylsulfanyl)pyrazolo[1,5-a][1,3,5]triazine (Intermediate 7, 120 mg, 429 μmol) and 1-[4-(4-methylphenyl)-1H-imidazol-2-yl]methanamine (121 mg, 644 μmol, CAS 1156713-02-8) were dissolved in n-butanol (2.6 mL), N,N-diisopropylethylamine (300 μL, 1.7 mmol) was added and the mixture was stirred for 3 h at 100° C. in the microwave. The mixture was evaporated, diluted with dichloromethane and water. The phases were separated and the aqueous phase was extracted 3 times with a mixture of dichloromethane and 2-propanol (4:1). The combined organic layers were dried, evaporated and purified by flash chromatography (dichloromethane/ethanol gradient 0-5% ethyl acetate) to give 172 mg (90% purity, 84% yield) the title compound.

LC-MS (Method 1): Rₜ=0.99 min; MS (ESIpos): m/z=430 [M+H]⁺

¹H-NMR (400 MHz, DMSO-d6) δ[ppm]: 0.884 (0.51), 0.902 (1.11), 0.920 (0.61), 1.053 (0.40), 1.071 (0.81), 1.159 (0.54), 2.065 (1.52), 2.278 (7.76), 2.481 (16.00), 2.518 (1.88), 2.523 (1.52), 4.764 (6.50), 5.758 (3.53), 7.117 (1.77), 7.136 (1.99), 7.473 (2.01), 7.612 (2.07), 7.632 (1.90), 8.250 (8.91), 9.385 (0.56), 11.887 (0.67).

### Intermediate 28

### 8-bromo-2-(methanesulfonyl)-N-{[4-(4-methylphenyl)-1H-imidazol-2-yl]methyl}pyrazolo[1,5-a][1,3,5]triazin-4-amine

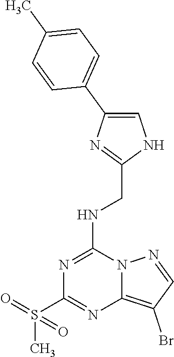 [SMILES: Cc1ccc(-c2c[nH]c(CNc3nc(S(C)(=O)=O)nc4c(Br)cnn34)n2)cc1]

8-bromo-N-{[4-(4-methylphenyl)-1H-imidazol-2-yl]methyl}-2-(methylsulfanyl)pyrazolo[1,5-a][1,3,5]triazin-4-amine (Intermediate 27, 172 mg, 400 μmol) was dissolved in acetonitrile (8.0 mL), cooled to 0° C., mCPBA (276 mg, 75% purity, 1.20 mmol) was added and the mixture was stirred for 16 h at rt. The mixture was diluted with dichloromethane, the layers separated and the aqueous phase was extracted 3 times with dichloromethane. The combined organic layers were dried and evaporated to give 375 mg of the title compound, which was used without further purification.

LC-MS (Method 1): Rₜ=0.91 min; MS (ESIpos): m/z=462 [M+H]⁺

### Intermediate 29

### 2-{[8-bromo-2-(methylsulfanyl)pyrazolo[1,5-a][1,3,5]triazin-4-yl]amino}ethan-1-ol

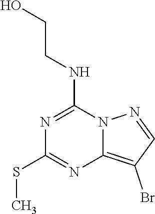 [SMILES: CSc1nc(NCCO)n2ncc(Br)c2n1]

8-bromo-4-chloro-2-(methylsulfanyl)pyrazolo[1,5-a][1,3,5]triazine (Intermediate 7, 1.20 g, 4.29 mmol) and 2-aminoethan-1-ol (393 mg, 6.44 mmol) were dissolved in n-butanol (12 mL), N,N-diisopropylethylamine (3.0 mL, 17 mmol) was added and the mixture was stirred for 16 h at 100° C. The mixture was evaporated, diluted with dichloromethane and water. The phases were separated and the aqueous phase was extracted 3× with a mixture of dichloromethane and 2-propanol (4:1). The combined organic layers were dried, evaporated to give 1.69 g of the title compound which was used without further purification.

LC-MS (Method 1): Rₜ=0.95 min; MS (ESIpos): m/z=304 [M+H]⁺

### Intermediate 30

### 2-{[8-bromo-2-(methanesulfonyl)pyrazolo[1,5-a][1,3,5]triazin-4-yl]amino}ethan-1-ol

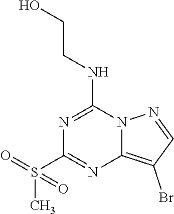 [SMILES: CS(=O)(=O)c1nc(NCCO)n2ncc(Br)c2n1]

2-{[8-bromo-2-(methylsulfanyl)pyrazolo[1,5-a][1,3,5]triazin-4-yl]amino}ethan-1-ol (Intermediate 29, 1.69 g, 5.56 mmol) was dissolved in acetonitrile (100 mL), cooled to 0° C., mCPBA (1.92 g, 75% purity, 8.33 mmol) was added and the mixture was stirred for 2 h at rt. The mixture was diluted with dichloromethane, the layers separated and the aqueous phase was extracted 3 times with dichloromethane. The combined organic layers were dried and evaporated to give 3.09 g of the title compound, which was used without further purification.

LC-MS (Method 1): Rₜ=0.68 min; MS (ESIpos): m/z=336 [M+H]⁺

### Intermediate 31

### 2-{[8-bromo-2-(morpholin-4-yl)pyrazolo[1,5-a][1,3,5]triazin-4-yl]amino}ethan-1-ol

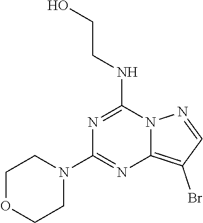 [SMILES: OCCNc1nc(N2CCOCC2)nc2c(Br)cnn12]

2-{[8-bromo-2-(methanesulfonyl)pyrazolo[1,5-a][1,3,5]triazin-4-yl]amino}ethan-1-ol (Intermediate 30, 3.09 g, 9.19 mmol) and morpholine (2.4 mL, 28 mmol) were dissolved in n-butanol (120 mL), N,N-diisopropylethylamine (16 mL, 92 mmol) was added and the mixture was stirred for 16 h at 100° C. in the microwave. The mixture was evaporated, diluted with dichloromethane and sat. sodium bicarbonate. The phases were separated and the aqueous phase was extracted 3× with a mixture of dichloromethane and 2-propanol (4:1). The combined organic layers were dried and evaporated. The residue was stirred in MeOH, the precipitate was collected by filtration, washed with MeOH and dried under reduced pressure to give 1.14 g (36% yield) of the title compound. The filtrate was evaporated and purified by flash chromatography (dichloromethane/ethanol gradient 0-5% ethyl acetate) to give another 250 mg (8% yield) the title compound.

LC-MS (Method 1): Rₜ=0.88 min; MS (ESIpos): m/z=343 [M+H]⁺

¹H-NMR (400 MHz, DMSO-d6) δ[ppm]: 0.916 (0.79), 0.934 (1.78), 0.953 (0.85), 2.331 (0.41), 2.518 (1.95), 2.522 (1.22), 3.490 (0.92), 3.506 (2.76), 3.520 (3.56), 3.533 (1.89), 3.573 (2.07), 3.586 (4.26), 3.600 (3.28), 3.618 (1.13), 3.632 (3.72), 3.643 (6.97), 3.655 (6.35), 3.672 (0.52), 3.705 (0.73), 3.721 (5.56), 3.733 (5.88), 3.744 (3.15), 4.271 (0.41), 4.288 (0.87), 4.786 (1.91), 4.800 (4.48), 4.814 (1.88), 4.825 (0.49), 5.758 (2.29), 7.971 (16.00), 8.149 (1.48), 8.368 (0.82), 8.382 (1.50), 8.396 (0.76).

### Intermediate 32

### {[8-bromo-2-(morpholin-4-yl)pyrazolo[1,5-a][1,3,5]triazin-4-yl]amino}acetaldehyde

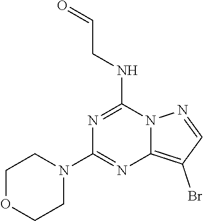 [SMILES: O=CCNc1nc(N2CCOCC2)nc2c(Br)cnn12]

2-{[8-bromo-2-(morpholin-4-yl)pyrazolo[1,5-a][1,3,5]triazin-4-yl]amino}ethan-1-ol (Intermediate 31, 100 mg, 291 μmol) was dissolved in dichloromethane (10 mL), Dess-Martin periodinane (163 mg, 350 μmol, CAS 87413-09-0) was added and the mixture was stirred for 19 h at room temperature. Dess-Martin periodinane (163 mg, 350 μmol, CAS 87413-09-0) was added and the mixture was stirred for another 16 h at room temperature. The mixture was diluted with sat. sodium bicarbonate. The phases were separated and the aqueous phase was extracted 3× with a mixture of dichloromethane and 2-propanol (4:1). The combined organic layers were dried and evaporated to give 150 mg the title compound which was used without further purification.

LC-MS (Method 1): Rₜ=0.88 min; MS (ESIpos): m/z=345 [M+H]⁺

### Intermediate 33

### N-[(7-methyl-3H-imidazo[4,5-b]pyridin-2-yl)methyl]-2-(methylsulfanyl)-8-(propan-2-yl)pyrazolo[1,5-a][1,3,5]triazin-4-amine

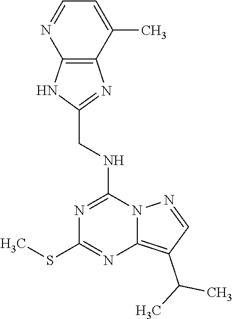 [SMILES: CSc1nc(NCc2nc3c(C)ccnc3[nH]2)n2ncc(C(C)C)c2n1]

4-chloro-2-(methylsulfanyl)-8-(propan-2-yl)pyrazolo[1,5-a][1,3,5]triazine (971 mg, 4.00 mmol, see European Journal of Medicinal Chemistry, 158, 1-6; 2018) and 1-(7-methyl-3H-imidazo[4,5-b]pyridin-2-yl)methanamine (973 mg, 6.00 mmol, CAS 1023813-52-6) were dissolved in acetonitrile (13 mL), N,N-diisopropylethylamine (3.3 mL, 19 mmol) was added and the mixture was stirred overnight at 50° C. The mixture was diluted with ethanol, the precipitate was collected by filtration, washed with water and dried under reduced pressure at 50° C. to give 636 mg (43% yield) of the title compound.

LC-MS (Method 2): Rₜ=1.11 min; MS (ESIpos): m/z=369 [M+H]⁺

¹H-NMR (400 MHz, DMSO-d6) δ[ppm]: 1.295 (16.00), 1.312 (15.56), 2.404 (15.15), 2.518 (4.56), 2.523 (2.94), 2.673 (0.80), 3.056 (0.95), 3.073 (1.25), 3.091 (0.91), 4.918 (4.78), 6.996 (1.56), 6.998 (1.58), 7.009 (1.61), 7.011 (1.61), 8.025 (4.92), 8.115 (1.02), 8.127 (1.01).

### Intermediate 34

### 2-(methanesulfonyl)-N-[(7-methyl-3H-imidazo[4,5-b]pyridin-2-yl)methyl]-8-(propan-2-yl)pyrazolo[1,5-a][1,3,5]triazin-4-amine

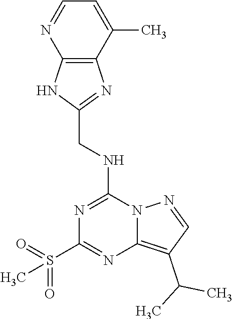 [SMILES: Cc1ccnc2[nH]c(CNc3nc(S(C)(=O)=O)nc4c(C(C)C)cnn34)nc12]

N-[(7-methyl-3H-imidazo[4,5-b]pyridin-2-yl)methyl]-2-(methylsulfanyl)-8-(propan-2-yl)pyrazolo[1,5-a][1,3,5]triazin-4-amine (Intermediate 33, 630 mg, 1.71 mmol) was dissolved in dichloromethane (11 mL), cooled to 0° C., mCPBA (1.26 g, 70% purity, 5.13 mmol) was added and the mixture was stirred for 3 h at rt. The mixture was diluted with sat. sodium bicarbonate solution, the phases were separated and the aqueous phase was extracted with a mixture of dichloromethane and 2-propanol (4:1). The combined organic phases were dried, and evaporated to give 210 mg of the title compound, which was used in the next step without further purification.

### Intermediate 35

### methyl N-[8-bromo-2-(methylsulfanyl)pyrazolo[1,5-a][1,3,5]triazin-4-yl]glycinate

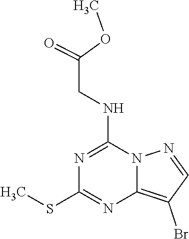 [SMILES: COC(=O)CNc1nc(SC)nc2c(Br)cnn12]

8-bromo-4-chloro-2-(methylsulfanyl)pyrazolo[1,5-a][1,3,5]triazine (Intermediate 7, 1.12 g, 4.00 mmol) and methyl glycinate hydrochloride (753 mg, 6.00 mmol) were dissolved in n-butanol (24 mL), N,N-diisopropylethylamine (2.8 mL, 16 mmol) was added and the mixture was stirred for 30 min at 90° C. in the microwave. The mixture was diluted with ethanol and water and the precipitate was collected by filtration, washed with water and ethanol and dried under reduced pressure to give 1.05 g (77% yield) of the title compound.

LC-MS (Method 2): Rₜ=1.08 min; MS (ESIpos): m/z=332 [M+H]⁺

¹H-NMR (400 MHz, DMSO-d6) δ[ppm]: 2.486 (16.00), 2.518 (1.22), 2.522 (0.77), 3.682 (13.16), 4.239 (5.20), 8.275 (5.51).

### Intermediate 36

### methyl N-[8-bromo-2-(methanesulfonyl)pyrazolo[1,5-a][1,3,5]triazin-4-yl]glycinate

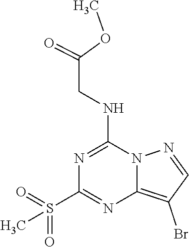 [SMILES: COC(=O)CNc1nc(S(C)(=O)=O)nc2c(Br)cnn12]

methyl N-[8-bromo-2-(methylsulfanyl)pyrazolo[1,5-a][1,3,5]triazin-4-yl]glycinate (Intermediate 35, 952 mg, 2.87 mmol) was dissolved in dichloromethane (24 mL), cooled to 0° C., mCPBA (1.48 g, 70% purity, 8.60 mmol) was added and the mixture was stirred for 2 h at rt. The mixture was diluted with dichloromethane, washed with sat. sodium bicarbonate solution, the aqueous phase was extracted 2 times with a mixture of dichloromethane and 2-propanol (4:1) and the combined organic layers were washed with water, dried and evaporated to give 1.1 g of the title compound, which was used without further purification.

¹H-NMR (400 MHz, DMSO-d6) δ[ppm]: 2.518 (0.78), 2.522 (0.52), 3.329 (16.00), 3.333 (8.64), 4.373 (2.31), 8.572 (2.75).

### Intermediate 37

### methyl N-[8-bromo-2-(morpholin-4-yl)pyrazolo[1,5-a][1,3,5]triazin-4-yl]glycinate

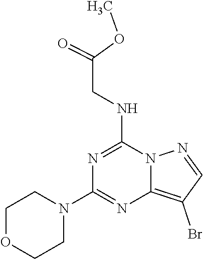 [SMILES: COC(=O)CNc1nc(N2CCOCC2)nc2c(Br)cnn12]

methyl N-[8-bromo-2-(methanesulfonyl)pyrazolo[1,5-a][1,3,5]triazin-4-yl]glycinate (Intermediate 36, 1.10 g, 3.02 mmol) and morpholine (790 μL, 9.1 mmol; CAS 110-91-8) were dissolved in acetonitrile (29 mL), N,N-diisopropylethylamine (1.6 mL, 9.1 mmol) was added and the mixture was stirred overnight at 70° C. The mixture was diluted with water and extracted with a mixture of dichloromethane and 2-propanol (4:1). The organic layer was dried, evaporated and the residue was stirred in ethanol. The precipitate was collected by filtration and dried to give 895 mg (78% yield) of the title compound.

LC-MS (Method 2): Rₜ=1.00 min; MS (ESIpos): m/z=371 [M+H]⁺

¹H-NMR (400 MHz, DMSO-d6) δ[ppm]: 2.075 (1.30), 2.518 (2.83), 2.523 (1.80), 2.673 (0.42), 3.614 (1.79), 3.624 (3.76), 3.636 (3.50), 3.659 (16.00), 3.677 (3.37), 3.689 (3.50), 3.699 (1.74), 4.170 (5.02), 8.028 (6.17), 8.977 (1.16).

### Intermediate 38

### N-[8-bromo-2-(morpholin-4-yl)pyrazolo[1,5-a][1,3,5]triazin-4-yl]glycine

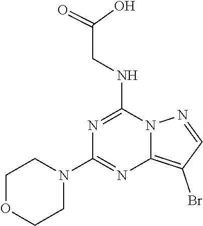 [SMILES: O=C(O)CNc1nc(N2CCOCC2)nc2c(Br)cnn12]

Methyl N-[8-bromo-2-(morpholin-4-yl)pyrazolo[1,5-a][1,3,5]triazin-4-yl]glycinate (Intermediate 37, 840 mg, 2.26 mmol) was dissolved in ethanol (10 mL) and tetrahydrofurane (5.0 mL), aqueous lithium hydroxide (11 mL, 1.0 M, 11 mmol) was added and the mixture was stirred for 48 h at rt. The reaction mixture was diluted with water and citric acid (2.17 g, 11.3 mmol). The mixture was stirred for 15 min at rt. The precipitate was collected by filtration, washed with ethanol and water and dried under reduced pressure at 50° C. to give 634 mg (78% yield) of the title compound.

LC-MS (Method 2): Rₜ=0.58 min; MS (ESIneg): m/z=355 [M−H]⁻

¹H-NMR (400 MHz, DMSO-d6) δ[ppm]: 2.074 (0.54), 2.518 (4.08), 2.522 (2.53), 3.599 (0.56), 3.616 (4.11), 3.626 (7.93), 3.638 (7.21), 3.695 (7.32), 3.707 (7.90), 3.717 (4.08), 4.068 (5.97), 4.082 (5.99), 8.018 (16.00), 8.782 (1.18), 8.796 (2.45), 8.812 (1.18), 12.844 (0.53).

### Intermediate 39

### 2-{[8-bromo-2-(morpholin-4-yl)pyrazolo[1,5-a][1,3,5]triazin-4-yl]amino}acetohydrazide

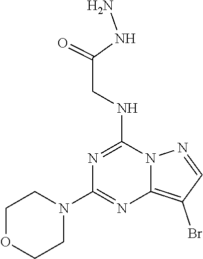 [SMILES: NNC(=O)CNc1nc(N2CCOCC2)nc2c(Br)cnn12]

N-[8-bromo-2-(morpholin-4-yl)pyrazolo[1,5-a][1,3,5]triazin-4-yl]glycine (Intermediate 38, 454 mg, 1.27 mmol) was dissolved in tetrahydrofuran (21 mL), di(1H-imidazol-1-yl)methanone (412 mg, 2.54 mmol; CAS 530-62-1) was added and the mixture was stirred overnight at reflux. hydrazine (6.4 mL, 1.0 M, 6.4 mmol) was added at room temperature and the mixture was stirred for 24 h at room temperature. The precipitate was collected by filtration, washed with ethanol and water and dried under reduced pressure at 50° C. to give 421 mg (85% purity, 76% yield) of the title compound.

LC-MS (Method 2): Rₜ=0.76 min; MS (ESIpos): m/z=371 [M+H]⁺

### Intermediate 40

### 2-(methylsulfanyl)-N-[(5-phenyl-1H-imidazol-2-yl)methyl]-8-(propan-2-yl)pyrazolo[1,5-a][1,3,5]triazin-4-amine

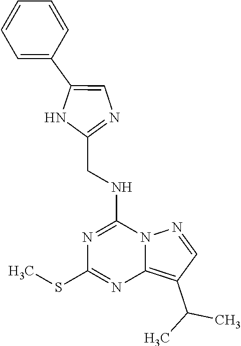 [SMILES: CSc1nc(NCc2ncc(-c3ccccc3)[nH]2)n2ncc(C(C)C)c2n1]

4-chloro-2-(methylsulfanyl)-8-(propan-2-yl)pyrazolo[1,5-a][1,3,5]triazine (728 mg, 3.00 mmol, see European Journal of Medicinal Chemistry, 158, 1-6; 2018) and 1-(5-phenyl-1H-imidazol-2-yl)methanamine (779 mg, 4.50 mmol) were dissolved in acetonitrile (9.6 mL), N,N-diisopropylethylamine (2.5 mL, 14 mmol) was added and the mixture was stirred overnight at 50° C. The mixture was poured into water, extracted 3 times with a mixture of dichloromethane and 2-propanol (4:1) and the combined organic layers were dried and evaporated. The residue was purified by flash chromatography (hexane/ethyl acetate gradient 50-100% ethyl acetate) to give 930 mg (80% yield) of the title compound.

LC-MS (Method 2): Rₜ=1.32 min; MS (ESIpos): m/z=380 [M+H]⁺

¹H-NMR (400 MHz, DMSO-d6) δ[ppm]: 1.154 (0.58), 1.172 (1.28), 1.190 (0.66), 1.229 (0.63), 1.246 (0.64), 1.288 (15.82), 1.306 (16.00), 1.987 (2.10), 2.454 (15.71), 2.518 (2.31), 2.522 (1.44), 3.048 (0.95), 3.065 (1.24), 3.083 (0.89), 4.017 (0.49), 4.035 (0.48), 4.739 (2.65), 4.754 (2.64), 7.160 (0.51), 7.303 (0.63), 7.320 (1.06), 7.338 (0.65), 7.519 (0.53), 7.720 (0.81), 7.736 (0.72), 8.002 (5.92), 9.120 (0.44).

### Intermediate 41

### 2-(methanesulfonyl)-N-[(5-phenyl-1H-imidazol-2-yl)methyl]-8-(propan-2-yl)pyrazolo[1,5-a][1,3,5]triazin-4-amine

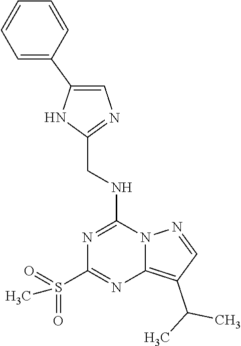 [SMILES: CC(C)c1cnn2c(NCc3ncc(-c4ccccc4)[nH]3)nc(S(C)(=O)=O)nc12]

2-(methylsulfanyl)-N-[(5-phenyl-1H-imidazol-2-yl)methyl]-8-(propan-2-yl)pyrazolo[1,5-a][1,3,5]triazin-4-amine (Intermediate 40, 740 mg, 1.95 mmol) was dissolved in dichloromethane (13 mL), cooled to 0° C., mCPBA (1.44 g, 70% purity, 5.85 mmol) was added and the mixture was stirred for 3 h at rt. The mixture was diluted with sat. sodium bicarbonate solution, the aqueous phase was extracted 3 times with a mixture of dichloromethane and 2-propanol (4:1) and the combined organic layers were dried and evaporated to give 980 mg of the title compound, which was used without further purification.

LC-MS (Method 1): Rₜ=0.92 min; MS (ESIpos): m/z=412 [M+H]⁺

### Intermediate 42

### 2-(methylsulfanyl)-N-[(5-phenyl-4H-1,2,4-triazol-3-yl)methyl]-8-(propan-2-yl)pyrazolo[1,5-a][1,3,5]triazin-4-amine

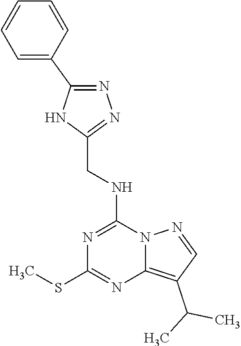 [SMILES: CSc1nc(NCc2nnc(-c3ccccc3)[nH]2)n2ncc(C(C)C)c2n1]

4-chloro-2-(methylsulfanyl)-8-(propan-2-yl)pyrazolo[1,5-a][1,3,5]triazine (728 mg, 3.00 mmol, see European Journal of Medicinal Chemistry, 158, 1-6; 2018) and 1-(5-phenyl-4H-1,2,4-triazol-3-yl)methanamine dihydrochloride (890 mg, 3.60 mmol, CAS 1337882-06-0) were dissolved in acetonitrile (9.6 mL), N,N-diisopropylethylamine (2.5 mL, 14 mmol) was added and the mixture was stirred overnight at 50° C. The mixture was diluted with water and extracted 3 times with A mixture of dichloromethane and 2-propanol (4:1). The combined organic layers were dried and evaporated to give 782 mg (68% yield) of the title compound.

LC-MS (Method 2): Rₜ=1.04 min; MS (ESIpos): m/z=381 [M+H]⁺

¹H-NMR (400 MHz, DMSO-d6) δ[ppm]: 1.052 (0.56), 1.288 (16.00), 1.305 (15.28), 2.074 (1.51), 2.433 (14.20), 2.518 (3.17), 2.523 (1.99), 3.048 (0.93), 3.065 (1.24), 3.082 (0.89), 4.806 (3.46), 7.419 (0.89), 7.436 (1.11), 7.449 (1.84), 7.468 (2.14), 7.485 (0.77), 7.489 (0.52), 7.950 (2.23), 7.954 (2.82), 7.958 (1.32), 7.966 (0.79), 7.971 (2.64), 7.974 (2.00), 8.016 (5.45).

### Intermediate 43

### 2-(methanesulfonyl)-N-[(5-phenyl-4H-1,2,4-triazol-3-yl)methyl]-8-(propan-2-yl)pyrazolo[1,5-a][1,3,5]triazin-4-amine

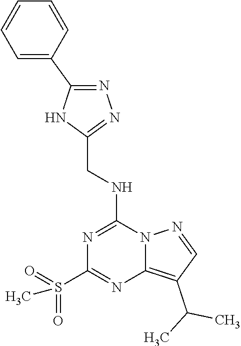 [SMILES: CC(C)c1cnn2c(NCc3nnc(-c4ccccc4)[nH]3)nc(S(C)(=O)=O)nc12]

2-(methylsulfanyl)-N-[(5-phenyl-4H-1,2,4-triazol-3-yl)methyl]-8-(propan-2-yl)pyrazolo[1,5-a][1,3,5]triazin-4-amine (Intermediate 42, 645 mg, 1.70 mmol) was dissolved in dichloromethane (30 mL), cooled to 0° C., mCPBA (644 mg, 70% purity, 3.73 mmol) was added and the mixture was stirred for 12 h at rt. The mixture was diluted with a mixture of dichloromethane and 2-propanol (4:1), washed with water, dried and evaporated to give 880 mg of the title compound in a mixture with the corresponding sulfone, which was used without further purification.

LC-MS (Method 1): Rₜ=1.04 min; MS (ESIpos): m/z=413 [M+H]⁺; Sulfone: Rₜ=0.98 min; MS (ESIpos): m/z=398 [M+H]⁺

### Intermediate 44

### {[8-bromo-2-(methylsulfanyl)pyrazolo[1,5-a][1,3,5]triazin-4-yl]amino}acetonitrile

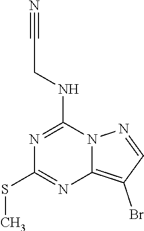 [SMILES: CSc1nc(NCC#N)n2ncc(Br)c2n1]

8-bromo-4-chloro-2-(methylsulfanyl)pyrazolo[1,5-a][1,3,5]triazine (Intermediate 7, 1.20 g, 4.29 mmol) and aminoacetonitrile hydrochloride (596 mg, 6.44 mmol) were dissolved in n-butanol (30 mL), N,N-diisopropylethylamine (3.0 mL, 17 mmol) was added and the mixture was stirred for 19 h at 100° C. The mixture was evaporated and purified by flash chromatography (dichloromethane/ethanol gradient 0-6% ethanol) to give 722 mg (56% yield) the title compound.

LC-MS (Method 1): Rₜ=1.04 min; MS (ESIneg): m/z=297 [M−H]⁻

¹H-NMR (400 MHz, DMSO-d6) δ[ppm]: 2.448 (0.55), 2.518 (5.83), 2.522 (3.66), 2.559 (16.00), 3.687 (0.50), 3.704 (0.45), 4.534 (6.50), 8.197 (0.95), 8.275 (5.32).

### Intermediate 45

### {[8-bromo-2-(methanesulfonyl)pyrazolo[1,5-a][1,3,5]triazin-4-yl]amino}acetonitrile

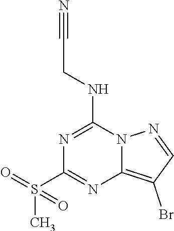 [SMILES: CS(=O)(=O)c1nc(NCC#N)n2ncc(Br)c2n1]

{[8-bromo-2-(methylsulfanyl)pyrazolo[1,5-a][1,3,5]triazin-4-yl]amino}acetonitrile (Intermediate 44, 722 mg, 2.41 mmol) was dissolved in acetonitrile (50 mL), cooled to 0° C., mCPBA (1.11 g, 75% purity, 4.83 mmol) was added and the mixture was stirred for 16 h at rt. The mixture was diluted with dichloromethane, the layers separated and the aqueous phase was extracted 3 times with dichloromethane. The combined organic layers were dried and evaporated to give 1.54 g of the title compound, which was used without further purification.

LC-MS (Method 1): Rₜ=0.75 min; MS (ESIpos): m/z=331 [M+H]⁺

### Intermediate 46

### {[8-bromo-2-(morpholin-4-yl)pyrazolo[1,5-a][1,3,5]triazin-4-yl]amino}acetonitrile

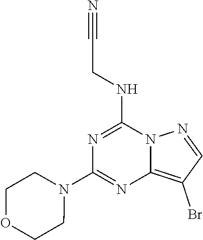 [SMILES: N#CCNc1nc(N2CCOCC2)nc2c(Br)cnn12]

{[8-bromo-2-(methanesulfonyl)pyrazolo[1,5-a][1,3,5]triazin-4-yl]amino}acetonitrile (Intermediate 45, 1.54 g, 4.65 mmol) and morpholine (1.2 mL, 14 mmol) were dissolved in n-butanol (40 mL), N,N-diisopropylethylamine (8.1 mL, 47 mmol) was added and the mixture was stirred for 16 h at 100° C. The mixture was evaporated, diluted with dichloromethane and water. The phases were separated and the aqueous phase was extracted 3 times with a mixture of dichloromethane and 2-propanol (4:1). The combined organic layers were dried, evaporated and purified by flash chromatography (dichloromethane/ethanol gradient 0-5% ethanol) to give 592 mg (37% yield) the title compound.

LC-MS (Method 1): Rₜ=0.96 min; MS (ESIpos): m/z=338 [M+H]⁺

### Intermediate 47

### {[8-bromo-2-(morpholin-4-yl)pyrazolo[1,5-a][1,3,5]triazin-4-yl]amino}ethanimidamide

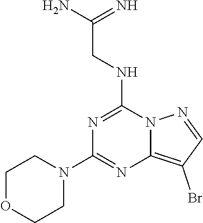 [SMILES: N=C(N)CNc1nc(N2CCOCC2)nc2c(Br)cnn12]

Ammonium chloride (44.3 mg, 828 μmol) was dissolved in toluene (5.0 mL), trimethylaluminium (410 μL, 2.0 M in toluene, 830 μmol) was added and the mixture was stirred for 10 min at room temperature. {[8-bromo-2-(morpholin-4-yl)pyrazolo[1,5-a][1,3,5]triazin-4-yl]amino}acetonitrile (Intermediate 46, 140 mg, 414 μmol) dissolved in toluene (5.0 mL) was added and the mixture was stirred for 16 h at 80° C. Ammonium chloride (88.6 mg, 1.66 mmol) and trimethylaluminium (820 μL, 2.0 M in toluene, 1.66 mmol) were added and the mixture was stirred for 3 h at 80° C. The mixture was quenched with sodium sulfate and stirred for 1 h at room temperature. The precipitate was collected by filtration, washed with dichloromethane, stirred with methanol and then collected by filtration, and dried under reduced pressure to give 205 mg of the title compound, which was used without further purification.

LC-MS (Method 1): Rₜ=0.67 min; MS (ESIpos): m/z=355 [M+H]⁺

¹H-NMR (400 MHz, DMSO-d6) δ[ppm]: 1.031 (3.28), 1.049 (6.21), 1.066 (3.28), 1.228 (1.11), 1.256 (0.55), 1.905 (0.51), 2.518 (10.09), 2.522 (6.34), 3.418 (0.72), 3.430 (0.72), 3.435 (0.68), 3.447 (0.64), 3.621 (3.74), 3.632 (6.89), 3.644 (6.26), 3.656 (1.19), 3.712 (6.17), 3.724 (6.64), 3.735 (3.57), 4.371 (0.43), 4.385 (0.60), 4.418 (9.57), 7.403 (6.43), 8.032 (16.00), 8.045 (0.64).

### Intermediate 48

### {[8-bromo-2-(morpholin-4-yl)pyrazolo[1,5-a][1,3,5]triazin-4-yl]amino}ethanimidamide hydrogen chloride (1/1)

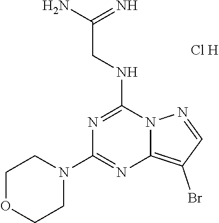 [SMILES: N=C(N)CNc1nc(N2CCOCC2)nc2c(Br)cnn12 ; "Cl H"]

Ammonium chloride (1.11 g, 20.8 mmol) was suspended in toluene (100 mL), trimethylalumane (10.4 mL, 2.0 M in toluene, 20.8 mmol) were added and the mixture was stirred for 20 min at rt. {[8-Bromo-2-(morpholin-4-yl)pyrazolo[1,5-a][1,3,5]triazin-4-yl]amino}acetonitrile (Intermediate 46, 2.34 g, 6.92 mmol) was added and the mixture was stirred at 80° C. for 16 h. Additional ammonium chloride (1.11 g, 20.8 mmol) and trimethylalumane (10.4 mL, 2.0 M in toluene, 20.8 mmol) were added and the mixture was stirred at 80° C. for 6 h. After the reaction mixture cooled down, 10 g of silica gel and 20 mL of methanol were added and the mixture was stirred for 1 h at rt. The solids were removed by filtration and washed with methanol. The filtrate was concentrated to give 2.80 g (95% yield) of the title compound.

LC-MS (Method 1): Rₜ=0.65 min; MS (ESIneg): m/z=355 [M+H]⁺

¹H-NMR (400 MHz, DMSO-d6) δ[ppm]: 2.109 (2.94), 2.331 (0.80), 2.518 (3.71), 2.522 (2.59), 2.669 (1.15), 2.673 (0.82), 3.153 (7.04), 3.166 (7.08), 3.622 (4.14), 3.631 (7.31), 3.644 (6.43), 3.660 (1.28), 3.673 (0.86), 3.695 (1.05), 3.711 (6.57), 3.723 (6.77), 3.734 (3.62), 3.785 (0.43), 4.111 (0.52), 4.125 (1.28), 4.138 (1.22), 4.151 (0.43), 4.413 (10.30), 4.476 (0.76), 4.490 (0.74), 4.509 (1.21), 7.210 (1.13), 7.216 (1.26), 7.228 (1.42), 7.238 (1.42), 7.303 (5.54), 7.313 (3.27), 7.316 (3.46), 7.354 (2.62), 7.955 (0.66), 8.033 (16.00), 8.041 (0.45), 8.045 (1.71), 8.049 (3.25), 8.059 (0.41).

### Intermediate 49

### 8-bromo-N-[(4-methoxyphenyl)methyl]-N-({1-[(4-methoxyphenyl)methyl]-1H-benzimidazol-2-yl}methyl)-2-(methylsulfanyl)pyrazolo[1,5-a][1,3,5]triazin-4-amine

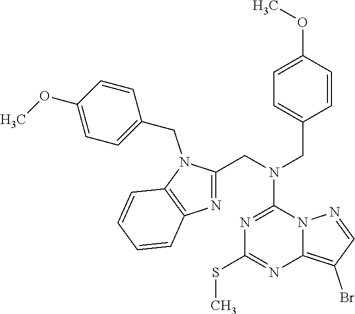 [SMILES: COc1ccc(CN(Cc2nc3ccccc3n2Cc2ccc(OC)cc2)c2nc(SC)nc3c(Br)cnn23)cc1]

N-[(1H-benzimidazol-2-yl)methyl]-8-bromo-2-(methylsulfanyl)pyrazolo[1,5-a][1,3,5]triazin-4-amine (Intermediate 8, 50.0 mg, 128 μmol) was provided in DMF (1 mL), potassium carbonate (35.4 mg, 256 μmol) and 1-(chloromethyl)-4-methoxybenzene (19 μL, 141 μmol; CAS-RN:[824-94-2]) were added and the reaction mixture was stirred for 90 min at rt, for 1 h at 60° C. and for 3 days at rt. In a second preparation, N-[(1H-benzimidazol-2-yl)methyl]-8-bromo-2-(methylsulfanyl)pyrazolo[1,5-a][1,3,5]triazin-4-amine (Intermediate 8, 50.0 mg, 128 μmol) was provided in DMF (1 mL), potassium carbonate (70.8 mg, 512 μmol) and 1-(chloromethyl)-4-methoxybenzene (38 μL, 280 μmol; CAS-RN:[824-94-2]) were added and the reaction mixture was stirred for 90 min at rt, for 1 h at 60° C. and for 3 days at rt. The two reaction mixtures were combined and poured into water. The precipitate was isolated by filtration and purified by flash chromatography using silica gel (dichlorormethane-ethylacetate gradient) to give 150 mg of the title compound.

LC-MS (Method 1): Rₜ=1.53 min; MS (ESIpos): m/z=630 [M+H]⁺

¹H-NMR (400 MHz, DMSO-d6) δ[ppm]: 1.155 (1.56), 1.173 (3.41), 1.190 (1.73), 1.232 (0.49), 1.988 (6.00), 2.459 (1.15), 2.518 (1.92), 2.523 (1.36), 3.689 (0.81), 3.702 (3.41), 3.712 (0.95), 3.725 (16.00), 3.739 (0.60), 3.741 (0.68), 4.000 (0.43), 4.017 (1.28), 4.035 (1.23), 4.397 (0.78), 4.411 (0.80), 5.022 (0.49), 5.037 (0.89), 5.051 (0.46), 5.433 (1.35), 6.841 (0.43), 6.859 (0.71), 6.865 (3.27), 6.870 (1.23), 6.887 (3.35), 6.895 (0.63), 6.912 (0.46), 7.033 (0.52), 7.150 (0.42), 7.158 (0.52), 7.174 (0.86), 7.190 (0.61), 7.210 (0.98), 7.232 (0.71), 7.281 (1.28), 7.302 (1.16), 7.478 (0.51), 7.496 (0.47).

### Intermediate 50

### 8-(3-fluorophenyl)-N-[(4-methoxyphenyl)methyl]-N-({1-[(4-methoxyphenyl)methyl]-1H-benzimidazol-2-yl}methyl)-2-(methylsulfanyl)pyrazolo[1,5-a][1,3,5]triazin-4-amine

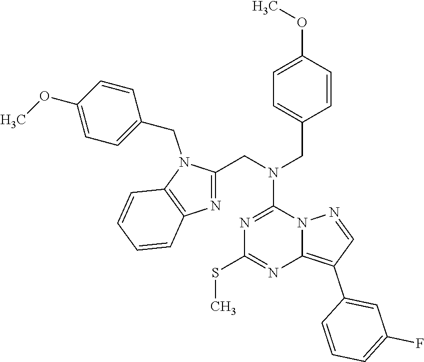 [SMILES: COc1ccc(CN(Cc2nc3ccccc3n2Cc2ccc(OC)cc2)c2nc(SC)nc3c(-c4cccc(F)c4)cnn23)cc1]

8-Bromo-N-[(4-methoxyphenyl)methyl]-N-({1-[(4-methoxyphenyl)methyl]-1H-benzimidazol-2-yl}methyl)-2-(methylsulfanyl)pyrazolo[1,5-a][1,3,5]triazin-4-amine (Intermediate 49, 145 mg) and 2-(3-fluorophenyl)-4,4,5,5-tetramethyl-1,3,2-dioxaborolane (76.6 mg, 345 μmol; CAS-RN:[936618-92-7]) were provided in tetrahydrofurane (6.9 mL), potassium phosphate (171 mg, 805 μmol) and water (0.4 mL) were added and the mixture was flushed with argon for 5 min. [1,1′-Bis(diphenylphosphino)ferrocene]dichloropalladium(II) dichloromethane complex (18.8 mg, 23.0 μmol; CAS-RN:[95464-05-4]) was added under argon and the mixture was stirred for 1 h at 130° C. in a microwave. The mixture was concentrated and purified by flash chromatography using silica gel (hexane-ethylacetate gradient) to give 126 mg of the title compound.

LC-MS (Method 1): Rₜ=1.64 min; MS (ESIpos): m/z=646 [M+H]⁺

¹H-NMR (400 MHz, DMSO-d6) δ[ppm]: 1.156 (1.62), 1.232 (0.57), 1.297 (11.00), 1.988 (0.55), 2.518 (2.81), 2.523 (2.51), 2.530 (1.54), 3.684 (1.04), 3.702 (0.94), 3.721 (0.91), 3.726 (8.21), 3.729 (16.00), 4.397 (0.88), 4.411 (0.92), 5.023 (0.45), 5.038 (0.83), 5.052 (0.42), 5.463 (1.47), 5.759 (0.60), 6.863 (1.38), 6.868 (0.88), 6.871 (0.82), 6.879 (3.50), 6.884 (2.27), 6.895 (1.26), 6.901 (3.26), 6.908 (0.55), 7.014 (0.41), 7.034 (0.78), 7.050 (0.69), 7.056 (0.81), 7.155 (0.56), 7.173 (0.93), 7.191 (0.66), 7.210 (1.16), 7.231 (0.78), 7.306 (1.68), 7.328 (1.59), 7.355 (0.47), 7.440 (0.82), 7.456 (0.83), 7.477 (0.65), 7.488 (0.77), 7.503 (0.62), 7.897 (0.73), 7.916 (1.11), 7.940 (0.53).

### Intermediate 51

### N-[(1H-benzimidazol-2-yl)methyl]-8-(3-fluorophenyl)-2-(methylsulfanyl)pyrazolo[1,5-a][1,3,5]triazin-4-amine

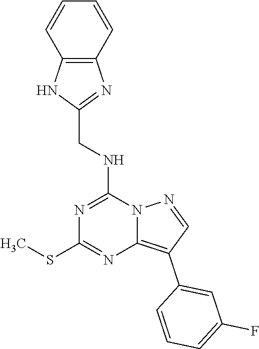 [SMILES: CSc1nc(NCc2nc3ccccc3[nH]2)n2ncc(-c3cccc(F)c3)c2n1]

8-(3-fluorophenyl)-N-[(4-methoxyphenyl)methyl]-N-({1-[(4-methoxyphenyl)methyl]-1H-benzimidazol-2-yl}methyl)-2-(methylsulfanyl)pyrazolo[1,5-a][1,3,5]triazin-4-amine (Intermediate 50, 110 mg) was dissolved in trifluoracetic acid and the mixture was stirred for 4 h at 150° C. in a microwave. The mixture was concentrated and purified by flash chromatography using silica gel (dichloromethane-ethylacetate-ethanol gradient) to give 63.0 mg of the title compound.

LC-MS (Method 1): Rₜ=1.10 min; MS (ESIpos): m/z=406 [M+H]⁺

¹H-NMR (400 MHz, DMSO-d6) δ[ppm]: 0.798 (1.09), 0.803 (0.53), 0.815 (1.07), 0.822 (1.09), 0.840 (0.63), 0.851 (0.63), 0.886 (0.58), 0.905 (1.14), 0.922 (0.58), 1.154 (1.04), 1.173 (1.85), 1.190 (0.99), 1.232 (2.26), 1.255 (0.69), 1.903 (0.43), 1.988 (2.26), 2.337 (0.51), 2.406 (0.86), 2.434 (1.73), 2.518 (7.14), 2.523 (4.57), 2.679 (0.51), 3.702 (0.41), 3.710 (1.50), 3.798 (0.56), 4.017 (0.48), 4.035 (0.48), 4.929 (0.66), 4.954 (8.38), 5.759 (3.28), 7.018 (0.99), 7.021 (0.99), 7.024 (1.09), 7.027 (1.12), 7.041 (2.06), 7.047 (2.21), 7.061 (1.12), 7.064 (1.12), 7.067 (1.17), 7.070 (1.19), 7.130 (2.18), 7.138 (3.38), 7.152 (3.66), 7.161 (2.34), 7.411 (1.42), 7.427 (1.35), 7.445 (1.65), 7.464 (2.51), 7.482 (2.44), 7.501 (1.42), 7.545 (1.50), 7.561 (1.32), 7.944 (4.09), 7.947 (5.94), 7.967 (5.61), 7.969 (3.76), 8.302 (0.69), 8.718 (0.41), 8.770 (16.00), 9.530 (1.07), 12.314 (1.57).

### Intermediate 52

### N-[(1H-benzimidazol-2-yl)methyl]-8-(3-fluorophenyl)-2-(methanesulfonyl)pyrazolo[1,5-a][1,3,5]triazin-4-amine

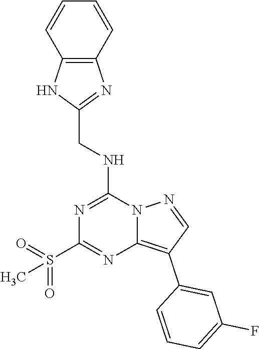 [SMILES: CS(=O)(=O)c1nc(NCc2nc3ccccc3[nH]2)n2ncc(-c3cccc(F)c3)c2n1]

N-[(1H-benzimidazol-2-yl)methyl]-8-(3-fluorophenyl)-2-(methylsulfanyl)pyrazolo[1,5-a][1,3,5]triazin-4-amine (Intermediate 51, 60.0 mg, 148 μmol) was provided in dichloromethane (500 μL) and m-chloroperoxybenzoic acid (99.5 mg, 77% purity, 444 μmol) was added at 0° C. The mixture was stirred for 4 h at rt, m-chloroperoxybenzoic acid (99.5 mg, 77% purity, 444 μmol) was added and stirring was continued for 2 h at rt. The mixture was purified by flash chromatography using silica gel (dichloromethane-methanol gradient) to give 40.0 mg of impure title compound which was used without further purification.

LC-MS (Method 1): Rₜ=0.95 min; MS (ESIpos): m/z=438 [M+H]⁺

¹H-NMR (400 MHz, DMSO-d6) δ[ppm]: 0.834 (0.47), 0.851 (0.94), 1.026 (0.55), 1.042 (0.55), 1.232 (2.98), 1.352 (0.47), 1.907 (1.10), 1.986 (0.47), 2.005 (0.39), 2.331 (3.29), 2.336 (1.49), 2.518 (16.00), 2.522 (10.67), 2.673 (3.29), 2.678 (1.41), 3.252 (0.63), 3.265 (1.33), 3.370 (1.25), 3.396 (1.18), 3.402 (1.96), 3.461 (0.86), 3.711 (0.47), 5.078 (2.04), 5.759 (3.14), 7.121 (0.94), 7.125 (1.02), 7.141 (2.27), 7.148 (2.75), 7.162 (2.27), 7.169 (1.65), 7.413 (0.86), 7.515 (1.18), 7.518 (1.41), 7.538 (2.75), 7.551 (1.96), 7.559 (2.27), 7.571 (1.41), 7.686 (0.94), 7.707 (0.94), 7.877 (1.10), 7.880 (2.04), 7.883 (1.57), 7.888 (2.20), 7.892 (2.59), 7.897 (3.37), 7.933 (1.18), 7.939 (1.10), 7.960 (1.25), 7.966 (1.18), 7.981 (1.65), 8.000 (1.33), 8.939 (0.71), 9.025 (7.37), 10.238 (0.63), 12.322 (0.94).

### Intermediate 53

### ethyl [(4-cyclopropyl-1H-pyrazol-5-yl)carbamothioyl]carbamate

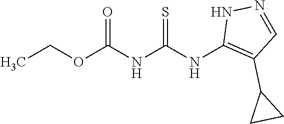 [SMILES: CCOC(=O)NC(=S)Nc1[nH]ncc1C1CC1]

A mixture of 4-cyclopropyl-1H-pyrazol-5-amine (5.83 g, 47.3 mmol; CAS-RN:[673475-74-6]) in ethylacetate (28 mL) under an argon atmosphere was stirred at 80° C., ethyl carbonisothiocyanatidate (5.9 mL, 50 mmol; CAS-RN:[16182-04-0]) was added dropwise and the mixture was stirred for 1 h at 80° C. The reaction mixture was cooled to 0° C. and the precipitate was isolated by filtration, washed with 20 mL of cold ethylacetate and dried to give 9.85 g (76% yield) of the title compound.

LC-MS (Method 2): Rₜ=0.79 min; MS (ESIpos): m/z=255 [M+H]⁺

¹H-NMR (400 MHz, DMSO-d6) δ[ppm]: 0.436 (1.08), 0.447 (3.53), 0.452 (3.73), 0.460 (3.97), 0.464 (3.62), 0.474 (1.31), 0.686 (1.12), 0.695 (2.90), 0.700 (2.95), 0.707 (1.88), 0.716 (3.14), 0.721 (2.96), 0.731 (1.04), 1.237 (7.58), 1.255 (16.00), 1.273 (7.62), 1.531 (0.75), 1.538 (0.88), 1.550 (1.23), 1.562 (0.82), 1.571 (0.65), 2.518 (1.08), 2.523 (0.72), 4.184 (2.18), 4.202 (6.61), 4.220 (6.52), 4.238 (2.06), 7.403 (2.61), 11.014 (2.02), 11.343 (1.34), 12.454 (1.79).

### Intermediate 54

### 8-cyclopropyl-2-(methylsulfanyl)pyrazolo[1,5-a][1,3,5]triazin-4-ol

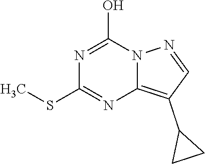

A mixture of ethyl [(4-cyclopropyl-1H-pyrazol-5-yl)carbamothioyl]carbamate (Intermediate 53, 9.85 g, 38.7 mmol) in an aqueous sodium hydroxid solution (37 mL, 2.0 M, 74 mmol) was stirred for 3 h at rt. The reaction mixture was diluted with ethanol (152 mL), iodomethane (2.4 mL, 38.7 mmol) was added and the mixture was stirred for 30 min at rt. The reaction mixture was cooled to 0° C., was diluted with water (100 mL) and stirred for 15 min. The precipitate was was removed by filtration. The filtrate was concentrated to half of the original volume and cooled to 0° C. The formed precipitate was removed by filtration. An aqueous sulfuric acid solution (39 mL, 1.0 M, 39 mmol) was added to the filtrate and the formed precipitate was isolated by filtration to give 1.80 g (21% yield) of the title compound.

LC-MS (Method 1): Rₜ=0.89 min; MS (ESIneg): m/z=223 [M+H]⁺

¹H-NMR (400 MHz, DMSO-d6) δ[ppm]: 0.785 (0.54), 0.791 (0.57), 0.797 (1.78), 0.803 (2.08), 0.810 (2.29), 0.815 (2.02), 0.824 (1.29), 0.844 (0.49), 0.858 (1.18), 0.865 (1.92), 0.871 (1.45), 0.878 (1.37), 0.883 (1.00), 0.887 (2.07), 0.892 (1.45), 0.896 (0.71), 0.904 (0.59), 1.810 (0.66), 1.819 (0.68), 1.824 (0.40), 1.831 (1.16), 1.839 (0.43), 1.844 (0.64), 1.852 (0.60), 2.522 (0.49), 2.542 (16.00), 7.786 (5.27), 12.699 (0.71).

### Intermediate 55

### 4-chloro-8-cyclopropyl-2-(methylsulfanyl)pyrazolo[1,5-a][1,3,5]triazine

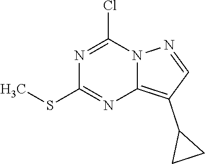

8-cyclopropyl-2-(methylsulfanyl)pyrazolo[1,5-a][1,3,5]triazin-4-ol (Intermediate 54, 1.70 g, 7.65 mmol) was provided under an argon atmosphere in pyridine (120 μL, 1.5 mmol) and phosphorus oxychloride (2.9 mL, 30.6 mmol) and the mixture was stirred for 23 h at 105° C. The reaction mixture was concentrated under reduced pressure and the remaining material was purified by flash chromatography using silica gel (hexane-ethylacetate-ethanol gradient) to give 266 mg (14% yield) of the title compound.

LC-MS (Method 1): Rₜ=1.26 min; MS (ESIpos): m/z=241 [M+H]⁺

¹H-NMR (400 MHz, CHLOROFORM-d) 5 [ppm]: 0.009 (2.50), 0.839 (0.42), 0.853 (1.22), 0.856 (0.95), 0.858 (1.36), 0.866 (1.74), 0.871 (1.51), 0.880 (0.87), 0.897 (0.46), 0.902 (1.00), 0.909 (1.02), 0.912 (1.00), 0.914 (0.91), 0.923 (0.98), 0.930 (0.96), 0.932 (1.11), 0.935 (0.79), 0.949 (0.46), 1.512 (8.35), 1.866 (0.50), 1.875 (0.53), 1.887 (0.70), 1.901 (0.45), 1.909 (0.42), 2.545 (16.00), 2.562 (0.42), 7.855 (3.19).

### Intermediate 56

### N-[(1H-benzimidazol-2-yl)methyl]-8-cyclopropyl-2-(methylsulfanyl)pyrazolo[1,5-a][1,3,5]triazin-4-amine

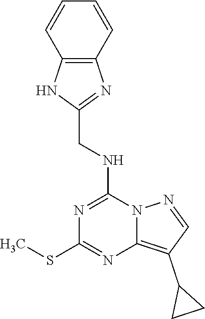 [SMILES: CSc1nc(NCc2nc3ccccc3[nH]2)n2ncc(C3CC3)c2n1]

1-(1H-benzimidazol-2-yl)methanamine hydrogen chloride (1/2) (242 mg, 1.10 mmol; CAS-RN:[5993-91-9]) was provided in dichloromethane (3.1 mL), N,N-diisopropylethylamine (380 μL, 2.2 mmol; CAS-RN:[7087-68-5]) and 4-chloro-8-cyclopropyl-2-(methylsulfanyl)pyrazolo[1,5-a][1,3,5]triazine (Intermediate 55, 265 mg, 1.10 mmol) were added and the reaction mixture was stirred for 15 min at rt. The mixture was concentrated and the remaining material was washed with water and ethanol to give 155 mg of the title compound.

LC-MS (Method 1): Rₜ=0.91 min; MS (ESIpos): m/z=352 [M+H]⁺

¹H-NMR (400 MHz, DMSO-d6) δ[ppm]: 0.795 (0.73), 0.808 (2.26), 0.813 (2.88), 0.821 (3.17), 0.825 (4.13), 0.830 (2.77), 0.834 (2.09), 0.837 (3.34), 0.842 (2.49), 0.850 (1.92), 0.857 (1.81), 0.866 (3.11), 0.874 (2.71), 0.880 (2.37), 0.882 (1.92), 0.885 (2.71), 0.891 (2.20), 0.896 (2.49), 0.901 (1.64), 0.909 (0.57), 0.913 (0.68), 1.052 (0.57), 1.232 (1.75), 1.838 (0.51), 1.850 (1.19), 1.859 (1.53), 1.867 (1.24), 1.871 (1.36), 1.880 (1.53), 1.884 (0.85), 1.893 (1.24), 1.901 (0.73), 1.907 (1.02), 2.331 (2.37), 2.336 (1.07), 2.369 (0.85), 2.393 (15.72), 2.421 (1.64), 2.431 (16.00), 2.451 (2.09), 2.518 (11.19), 2.522 (7.63), 2.543 (1.30), 2.596 (0.73), 2.669 (3.28), 2.673 (2.43), 2.678 (1.02), 3.428 (0.51), 4.967 (1.81), 4.981 (1.81), 7.252 (1.19), 7.376 (1.41), 7.385 (1.19), 7.391 (1.24), 7.400 (1.47), 7.545 (1.30), 7.552 (1.30), 7.743 (1.41), 7.752 (1.24), 7.758 (1.24), 7.767 (1.24), 7.952 (10.29), 9.295 (0.51), 9.310 (1.02), 9.325 (0.45), 10.429 (2.71).

### Intermediate 57

### N-[(1H-benzimidazol-2-yl)methyl]-8-cyclopropyl-2-(methanesulfonyl)pyrazolo[1,5-a][1,3,5]triazin-4-amine

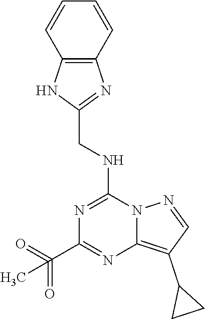

N-[(1H-benzimidazol-2-yl)methyl]-8-cyclopropyl-2-(methylsulfanyl)pyrazolo[1,5-a][1,3,5]triazin-4-amine (Intermediate 56, 150 mg) was provided in dichloromethane (1.4 mL), at 0° C. m-chloroperoxybenzoic acid (99.5 mg, 77% purity, 444 μmol; CAS-RN:[937-14-4]) was added and the mixture was stirred for 4 h at rt. Additional m-chloroperoxybenzoic acid (99.5 mg, 77% purity, 444 μmol) was added and the mixture was stirred for 2 h at rt. The reaction mixture was poured into saturated aqueous sodium thiosulfate solution and extracted with dichloromethane. The organic layers were combined, filtered over a water repellant filter and concentrated. The residue was purified by flash chromatography using silica gel (dichloromethane-methanol gradient) to give 189 mg of impure title compound which was used without further purification.

LC-MS (Method 1): Rₜ=0.79 min; MS (ESIpos): m/z=384 [M+H]⁺

### Intermediate 58

### 8-cyclopropyl-2-(methylsulfanyl)-N-[(5-phenyl-4H-1,2,4-triazol-3-yl)methyl]pyrazolo[1,5-a][1,3,5]triazin-4-amine

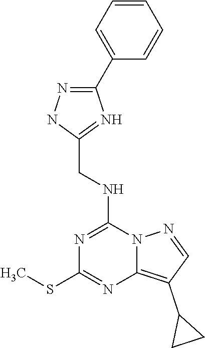 [SMILES: CSc1nc(NCc2nc(-c3ccccc3)n[nH]2)n2ncc(C3CC3)c2n1]

1-(5-Phenyl-4H-1,2,4-triazol-3-yl)methanamine hydrogen chloride (1/2) (250 mg, 1.01 mmol; CAS-RN:[1337882-06-0]) was provided in dichloromethane (5.0 mL), N,N-diisopropylethylamine (350 μL, 2.0 mmol) and 4-chloro-8-cyclopropyl-2-(methylsulfanyl)pyrazolo[1,5-a][1,3,5]triazine (Intermediate 55, 244 mg, 1.01 mmol) were added and the reaction mixture was stirred for 15 min at rt. The mixture was concentrated and the remaining material was washed with water and ethanol to give 180 mg (46% yield) of the title compound.

LC-MS (Method 1): Rₜ=1.20 min; MS (ESIpos): m/z=379 [M+H]⁺

¹H-NMR (400 MHz, DMSO-d6) δ[ppm]: 0.800 (2.46), 0.813 (7.72), 0.818 (8.58), 0.826 (11.60), 0.831 (8.41), 0.839 (4.57), 0.844 (2.07), 0.855 (5.18), 0.862 (6.56), 0.868 (5.00), 0.876 (4.31), 0.884 (7.03), 0.889 (4.53), 0.901 (1.77), 1.224 (1.08), 1.240 (3.36), 1.256 (4.44), 1.272 (2.20), 1.352 (0.22), 1.832 (0.95), 1.845 (2.54), 1.853 (2.54), 1.866 (2.89), 1.878 (1.81), 1.886 (1.60), 1.899 (0.73), 1.907 (1.04), 1.986 (0.22), 2.005 (0.22), 2.336 (0.86), 2.431 (16.00), 2.518 (9.10), 2.522 (6.08), 2.673 (1.85), 2.678 (0.82), 3.123 (0.26), 3.134 (0.26), 3.141 (0.30), 3.152 (0.26), 3.609 (0.22), 3.619 (0.17), 4.188 (0.22), 4.821 (2.11), 5.759 (4.79), 6.359 (0.30), 6.367 (0.65), 7.452 (2.93), 7.917 (11.21), 7.953 (5.87), 7.971 (5.13), 7.993 (0.73), 8.013 (0.47), 8.133 (0.26), 8.219 (0.60), 8.412 (0.17), 9.152 (0.39), 9.276 (0.78), 13.885 (0.73), 14.263 (0.35).

### Intermediate 59

### 8-cyclopropyl-2-(methanesulfonyl)-N-[(5-phenyl-4H-1,2,4-triazol-3-yl)methyl]pyrazolo[1,5-a][1,3,5]triazin-4-amine

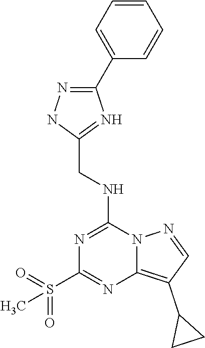 [SMILES: CS(=O)(=O)c1nc(NCc2nc(-c3ccccc3)n[nH]2)n2ncc(C3CC3)c2n1]

8-Cyclopropyl-2-(methylsulfanyl)-N-[(5-phenyl-4H-1,2,4-triazol-3-yl)methyl]pyrazolo[1,5-a][1,3,5]triazin-4-amine (Intermediate 58, 176 mg, 465 μmol) was provided in dichloromethane (1.6 mL), at 0° C. m-chloroperoxybenzoic acid (313 mg, 77% purity, 1.40 mmol; CAS-RN:[937-14-4]) was added and the mixture was stirred for 1.5 h at rt. The reaction mixture was poured into saturated aqueous sodium thiosulfate solution and extracted with dichloromethane. The organic layers were combined, filtered over a water repellant filter and concentrated. The residue was purified by flash chromatography using silica gel (dichloromethane-ethanol gradient) to give 219 mg of impure title compound which was used without further purification.

LC-MS (Method 1): Rₜ=0.98 min; MS (ESIpos): m/z=411 [M+H]⁺

### Intermediate 60

### 8-bromo-2-(methanesulfonyl)-N-[(4-methoxyphenyl)methyl]-N-({1-[(4-methoxyphenyl)methyl]-1H-benzimidazol-2-yl}methyl)pyrazolo[1,5-a][1,3,5]triazin-4-amine

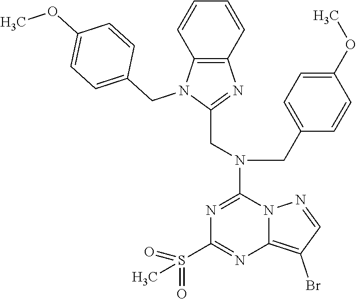 [SMILES: COc1ccc(CN(Cc2nc3ccccc3n2Cc2ccc(OC)cc2)c2nc(S(C)(=O)=O)nc3c(Br)cnn23)cc1]

8-Bromo-N-[(4-methoxyphenyl)methyl]-N-({1-[(4-methoxyphenyl)methyl]-1H-benzimidazol-2-yl}methyl)-2-(methylsulfanyl)pyrazolo[1,5-a][1,3,5]triazin-4-amine (Intermediate 49, 4.91 g, 7.79 mmol) was provided in dichloromethane (26 mL), at 0° C. m-chloroperoxybenzoic acid (5.24 g, 77% purity, 23.4 mmol; CAS-RN:[937-14-4]) was added in portions and the mixture was stirred for 2 days at rt. The reaction mixture was poured into saturated aqueous sodium thiosulfate solution and extracted with dichloromethane. The organic layers were combined, washed with an aqueous sodium bicarbonate solution, dried over sodium sulfate, filtered and concentrated. The residue was purified by flash chromatography using silica gel (dichloromethane-ethanol gradient) to give 4.68 g of impure title compound which was used without further purification.

LC-MS (Method 1): Rₜ=1.34 min; MS (ESIpos): m/z=662 [M+H]⁺

### Intermediate 61

### 8-bromo-N-[(4-methoxyphenyl)methyl]-N-({1-[(4-methoxyphenyl)methyl]-1H-benzimidazol-2-yl}methyl)-2-(morpholin-4-yl)pyrazolo[1,5-a][1,3,5]triazin-4-amine

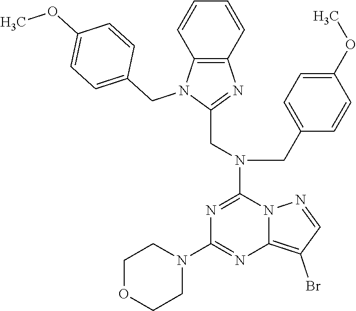 [SMILES: COc1ccc(CN(Cc2nc3ccccc3n2Cc2ccc(OC)cc2)c2nc(N3CCOCC3)nc3c(Br)cnn23)cc1]

8-Bromo-2-(methanesulfonyl)-N-[(4-methoxyphenyl)methyl]-N-({1-[(4-methoxyphenyl)methyl]-1H-benzimidazol-2-yl}methyl)pyrazolo[1,5-a][1,3,5]triazin-4-amine (Intermediate 60, 240 mg) was provided in acetonitrile (5.0 mL), morpholine (310 μL, 3.6 mmol; CAS-RN:[110-91-8]) was added and the reaction mixture was stirred for 2 h at 70° C. The reaction mixture was concentrated and purified by flash chromatography using silica gel (dichloromethane-ethanol gradient) to give 163 mg of impure title compound which was used without further purification.

LC-MS (Method 1): Rₜ=1.46 min; MS (ESIpos): m/z=669 [M+H]⁺

### Intermediate 62

### 8-(2-fluorophenyl)-N-[(4-methoxyphenyl)methyl]-N-({1-[(4-methoxyphenyl)methyl]-1H-benzimidazol-2-yl}methyl)-2-(morpholin-4-yl)pyrazolo[1,5-a][1,3,5]triazin-4-amine

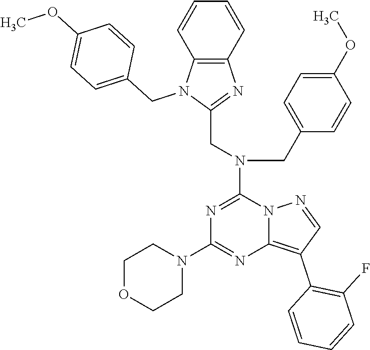 [SMILES: COc1ccc(CN(Cc2nc3ccccc3n2Cc2ccc(OC)cc2)c2nc(N3CCOCC3)nc3c(-c4ccccc4F)cnn23)cc1]

8-Bromo-N-[(4-methoxyphenyl)methyl]-N-({1-[(4-methoxyphenyl)methyl]-1H-benzimidazol-2-yl}methyl)-2-(morpholin-4-yl)pyrazolo[1,5-a][1,3,5]triazin-4-amine (Intermediate 61, 160 mg) and (2-fluorophenyl)boronic acid (50.2 mg, 358 μmol; CAS-RN:[1993-03-9]) were provided in tetrahydrofurane (2.5 mL), potassium phosphate (178 mg, 836 μmol) and water (0.5 mL) were added and the mixture was flushed with argon for 5 min. [1,1′-Bis(diphenylphosphino)ferrocene]dichloropalladium(II) dichloromethane complex (19.5 mg, 23.9 μmol; CAS-RN:[95464-05-4]) was added under argon and the mixture was stirred for 1 h at 130° C. in a microwave. The mixture was concentrated and purified by flash chromatography using silica gel (hexane-ethylacetate gradient) to give 95.0 mg of impure title compound which was used without further purification.

LC-MS (Method 1): Rₜ=1.56 min; MS (ESIpos): m/z=685 [M+H]⁺

### Intermediate 63

### 8-bromo-N-[(4-fluoro-1H-benzimidazol-2-yl)methyl]-2-(methylsulfanyl)pyrazolo[1,5-a][1,3,5]triazin-4-amine hydrogen chloride (1/1)

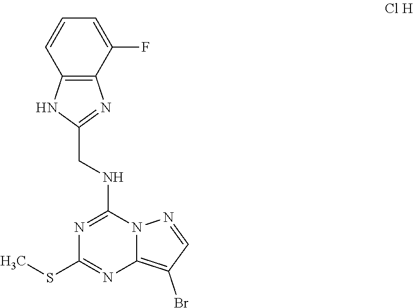 [SMILES: CSc1nc(NCc2nc3c(F)cccc3[nH]2)n2ncc(Br)c2n1]

1-(4-Fluoro-1H-benzimidazol-2-yl)methanamine hydrogen chloride (1/2) (1.26 g, 5.29 mmol; CAS-RN:[2089257-74-7]) was provided in dichloromethane (20 mL), N,N-diisopropylethylamine (1.8 mL, 11 mmol; CAS-RN:[7087-68-5]) and 8-bromo-4-chloro-2-(methylsulfanyl)pyrazolo[1,5-a][1,3,5]triazine (Intermediate 7, 1.48 g, 5.29 mmol) were added and the reaction mixture was stirred for 1 h at rt. The reaction mixture was concentrated and the remaining material was washed with water and dried to give 2.29 g (84% yield) of the title compound.

LC-MS (Method 2): Rₜ=1.11 min; MS (ESIpos): m/z=408 [M+H]⁺

¹H-NMR (400 MHz, DMSO-d6) δ[ppm]: 1.235 (0.77), 1.246 (2.78), 1.253 (1.70), 1.262 (2.99), 1.267 (2.86), 1.272 (1.10), 1.284 (2.64), 2.397 (0.46), 2.422 (16.00), 2.518 (3.38), 2.523 (2.34), 2.571 (2.78), 2.626 (0.40), 3.116 (0.46), 3.127 (0.48), 3.135 (0.47), 3.145 (0.46), 3.571 (1.58), 3.587 (1.71), 3.596 (1.69), 3.613 (1.70), 3.620 (1.59), 3.630 (1.52), 4.991 (2.86), 5.005 (2.87), 7.038 (0.68), 7.058 (0.93), 7.065 (0.74), 7.086 (0.85), 7.188 (0.58), 7.201 (0.65), 7.209 (1.16), 7.221 (1.14), 7.229 (0.69), 7.241 (0.60), 7.341 (1.96), 7.361 (1.46), 8.121 (1.10), 8.303 (7.82), 9.613 (0.70), 9.628 (1.44), 9.643 (0.63).

### Intermediate 64

### 8-bromo-N-[(4-fluoro-1H-benzimidazol-2-yl)methyl]-2-(methanesulfonyl)pyrazolo[1,5-a][1,3,5]triazin-4-amine

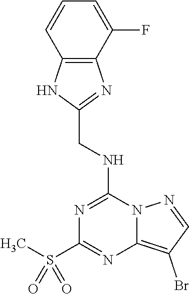 [SMILES: CS(=O)(=O)c1nc(NCc2nc3c(F)cccc3[nH]2)n2ncc(Br)c2n1]

8-bromo-N-[(4-fluoro-1H-benzimidazol-2-yl)methyl]-2-(methylsulfanyl)pyrazolo[1,5-a][1,3,5]triazin-4-amine hydrogen chloride (1/1) (Intermediate 63, 2.29 g, 5.15 mmol) was dissolved in dichloromethane (50 mL), m-chloroperoxybenzoic acid (3.46 g, 77% purity, 15.4 mmol; CAS-RN:[937-14-4]) was added and the mixture was stirred at rt over night. Additional m-chloroperoxybenzoic acid (3.46 g, 77% purity, 15.4 mmol) was added and the reaction mixture was stirred for 24 h at rt. A saturated aqueous sodium hydrogencarbonate solution was added and the mixture was stirred at pH 8 for 1 h at rt. The organic layer was separated, washed with an aqueous iron-II-sulfate solution, dried over sodium sulfate, filtrated and concentrated. The residue was purified by flash chromatography using silica gel (dichloromethane-ethanol gradient to give 1.75 g (77% yield) of impure title compound which was used without further purification.

LC-MS (Method 2): Rₜ=0.72 min; MS (ESIpos): m/z=440 [M+H]⁺

### Intermediate 65

### 8-bromo-N-[(5-fluoro-1H-benzimidazol-2-yl)methyl]-2-(methylsulfanyl)pyrazolo[1,5-a][1,3,5]triazin-4-amine hydrogen chloride (1/1)

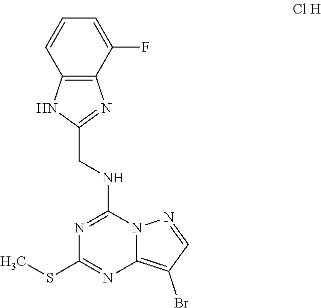 [SMILES: CSc1nc(NCc2nc3c(F)cccc3[nH]2)n2ncc(Br)c2n1]

8-bromo-4-chloro-2-(methylsulfanyl)pyrazolo[1,5-a][1,3,5]triazine (Intermediate 7, 1.31 g, 4.70 mmol) and 1-(5-fluoro-1H-benzimidazol-2-yl)methanamine hydrogen chloride (1/2) (1.12 g, 4.70 mmol; CAS-RN:[1216862-84-8]) were dissolved in dichloromethane (30 mL), N,N-diisopropylethylamine (1.6 mL, 9.4 mmol; CAS-RN:[7087-68-5]) was added and the solution was stirred for 2 h at rt. The reaction mixture was concentrated and the residue was washed with water and ethanol to give 1.40 g of impure title compound which was used without further purification.

LC-MS (Method 2): Rₜ=1.11 min; MS (ESIpos): m/z=408 [M+H]⁺

### Intermediate 66

### 8-bromo-N-[(5-fluoro-1H-benzimidazol-2-yl)methyl]-2-(methanesulfonyl)pyrazolo[1,5-a][1,3,5]triazin-4-amine

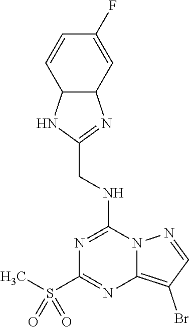 [SMILES: CS(=O)(=O)c1nc(NCC2=NC3C=C(F)C=CC3N2)n2ncc(Br)c2n1]

8-bromo-N-[(5-fluoro-1H-benzimidazol-2-yl)methyl]-2-(methylsulfanyl)pyrazolo[1,5-a][1,3,5]triazin-4-amine hydrogen chloride (1/1) (Intermediate 65, 1.40 g) was dissolved in dichloromethane (30 mL), m-chloroperoxybenzoic acid (2.12 g, 77% purity, 9.44 mmol; CAS-RN:[937-14-4]) was added and the mixture was stirred at rt over night. Additional m-chloroperoxybenzoic acid (2.12 g, 77% purity, 9.44 mmol) was added and the reaction mixture was stirred for 24 h at rt. A saturated aqueous sodium hydrogencarbonate solution was added and the mixture was stirred at pH 8 for 1 h at rt. The organic layer was separated, washed with an aqueous iron-II-sulfate solution, dried over sodium sulfate, filtrated and concentrated to give 915 mg of impure title compound which was used without further purification.

LC-MS (Method 1): Rₜ=0.84 min; MS (ESIpos): m/z=440 [M+H]⁺

### Intermediate 67

### N-[(4-methoxyphenyl)methyl]-N-({1-[(4-methoxyphenyl)methyl]-1H-benzimidazol-2-yl}methyl)-2-(morpholin-4-yl)-8-phenylpyrazolo[1,5-a][1,3,5]triazin-4-amine

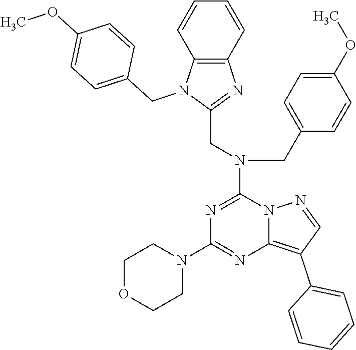 [SMILES: COc1ccc(CN(Cc2nc3ccccc3n2Cc2ccc(OC)cc2)c2nc(N3CCOCC3)nc3c(-c4ccccc4)cnn23)cc1]

In analogy to the procedure described in Intermediate 62 the reaction of 8-bromo-N-[(4-methoxyphenyl)methyl]-N-({1-[(4-methoxyphenyl)methyl]-1H-benzimidazol-2-yl}methyl)-2-(morpholin-4-yl)pyrazolo[1,5-a][1,3,5]triazin-4-amine (Intermediate 61, 250 mg) and 4,4,5,5-tetramethyl-2-phenyl-1,3,2-dioxaborolane (114 mg, 560 μmol; CAS-RN:[24388-23-6]) gave 166 mg of the title compound.

LC-MS (Method 1): Rₜ=1.53 min; MS (ESIpos): m/z=667 [M+H]⁺

¹H-NMR (400 MHz, DMSO-d6) δ[ppm]: 1.066 (16.00), 1.154 (0.80), 1.172 (1.66), 1.190 (0.85), 1.232 (0.24), 1.987 (3.04), 2.326 (0.33), 2.331 (0.23), 2.518 (1.17), 2.522 (0.81), 2.668 (0.33), 2.673 (0.23), 3.542 (0.17), 3.592 (0.35), 3.664 (0.83), 3.700 (0.36), 3.725 (6.13), 3.939 (2.63), 3.999 (0.24), 4.017 (0.71), 4.035 (0.70), 4.053 (0.23), 5.439 (1.17), 6.842 (0.20), 6.866 (0.25), 6.873 (1.19), 6.878 (0.43), 6.890 (0.45), 6.895 (1.26), 7.035 (0.21), 7.101 (0.24), 7.119 (0.49), 7.138 (0.35), 7.160 (0.27), 7.179 (0.42), 7.196 (0.28), 7.272 (0.58), 7.293 (0.52), 7.328 (0.58), 7.348 (0.88), 7.367 (0.50), 7.498 (0.23), 7.517 (0.21), 7.589 (0.21), 7.607 (0.19), 7.981 (0.78), 8.000 (0.72).

### Intermediate 68

### 8-(4-fluorophenyl)-N-[(4-methoxyphenyl)methyl]-N-({1-[(4-methoxyphenyl)methyl]-1H-benzimidazol-2-yl}methyl)-2-(morpholin-4-yl)pyrazolo[1,5-a][1,3,5]triazin-4-amine

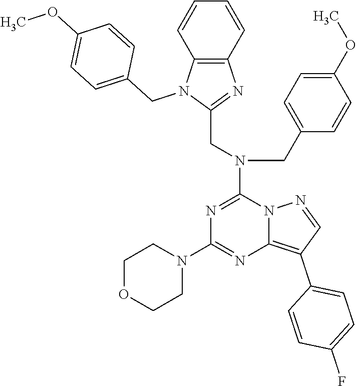 [SMILES: COc1ccc(CN(Cc2nc3ccccc3n2Cc2ccc(OC)cc2)c2nc(N3CCOCC3)nc3c(-c4ccc(F)cc4)cnn23)cc1]

In analogy to the procedure described in Intermediate 62 the reaction of 8-bromo-N-[(4-methoxyphenyl)methyl]-N-({1-[(4-methoxyphenyl)methyl]-1H-benzimidazol-2-yl}methyl)-2-(morpholin-4-yl)pyrazolo[1,5-a][1,3,5]triazin-4-amine (Intermediate 61, 250 mg) and 2-(4-fluorophenyl)-4,4,5,5-tetramethyl-1,3,2-dioxaborolane (124 mg, 560 μmol; CAS-RN:[214360-58-4]) gave 145 mg of impure title compound which was used without further purification.

LC-MS (Method 1): Rₜ=1.54 min; MS (ESIpos): m/z=685 [M+H]⁺

### Intermediate 69

### 8-(3-chlorophenyl)-N-[(4-methoxyphenyl)methyl]-N-({1-[(4-methoxyphenyl)methyl]-1H-benzimidazol-2-yl}methyl)-2-(morpholin-4-yl)pyrazolo[1,5-a][1,3,5]triazin-4-amine

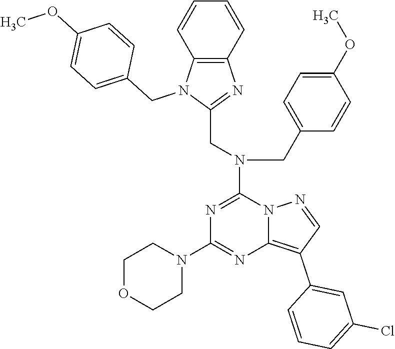 [SMILES: COc1ccc(CN(Cc2nc3ccccc3n2Cc2ccc(OC)cc2)c2nc(N3CCOCC3)nc3c(-c4cccc(Cl)c4)cnn23)cc1]

In analogy to the procedure described in Intermediate 62 the reaction of 8-bromo-N-[(4-methoxyphenyl)methyl]-N-({1-[(4-methoxyphenyl)methyl]-1H-benzimidazol-2-yl}methyl)-2-(morpholin-4-yl)pyrazolo[1,5-a][1,3,5]triazin-4-amine (Intermediate 61, 250 mg) and (3-chlorophenyl)boronic acid (87.6 mg, 560 μmol; CAS-RN:[63503-60-6]) gave 187 mg of the title compound.

LC-MS (Method 1): Rₜ=1.63 min; MS (ESIpos): m/z=701 [M+H]⁺

¹H-NMR (400 MHz, DMSO-d6) δ[ppm]: −0.149 (0.18), −0.008 (1.82), 0.008 (1.53), 0.146 (0.16), 0.854 (0.23), 1.157 (1.28), 1.175 (2.78), 1.192 (1.45), 1.234 (0.64), 1.261 (0.23), 1.989 (5.27), 2.334 (0.69), 2.338 (0.31), 2.520 (2.99), 2.525 (2.09), 2.676 (0.69), 2.680 (0.31), 3.308 (0.36), 3.545 (0.38), 3.661 (1.46), 3.702 (0.87), 3.728 (16.00), 4.002 (0.41), 4.019 (1.18), 4.037 (1.17), 4.055 (0.38), 5.440 (2.56), 6.840 (0.34), 6.868 (0.54), 6.875 (2.87), 6.880 (1.02), 6.892 (1.05), 6.897 (2.91), 7.025 (0.38), 7.145 (0.94), 7.148 (0.90), 7.150 (0.84), 7.165 (1.36), 7.168 (1.30), 7.182 (0.92), 7.198 (0.61), 7.215 (0.26), 7.273 (1.17), 7.293 (1.05), 7.353 (0.99), 7.373 (1.71), 7.393 (0.82), 7.499 (0.46), 7.515 (0.43), 7.591 (0.43), 7.610 (0.39), 7.974 (0.89), 7.995 (0.82), 8.073 (1.10), 8.078 (1.68), 8.082 (1.00), 8.378 (0.21).

### Intermediate 70

### N-[(4-methoxyphenyl)methyl]-N-({1-[(4-methoxyphenyl)methyl]-1H-benzimidazol-2-yl}methyl)-2-(morpholin-4-yl)-8-(pyridin-3-yl)pyrazolo[1,5-a][1,3,5]triazin-4-amine

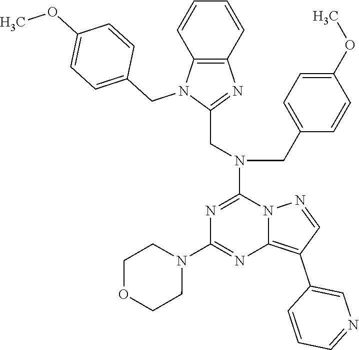 [SMILES: COc1ccc(CN(Cc2nc3ccccc3n2Cc2ccc(OC)cc2)c2nc(N3CCOCC3)nc3c(-c4cccnc4)cnn23)cc1]

In analogy to the procedure described in Intermediate 62 the reaction of 8-bromo-N-[(4-methoxyphenyl)methyl]-N-({1-[(4-methoxyphenyl)methyl]-1H-benzimidazol-2-yl}methyl)-2-(morpholin-4-yl)pyrazolo[1,5-a][1,3,5]triazin-4-amine (Intermediate 61, 250 mg) and 3-(4,4,5,5-tetramethyl-1,3,2-dioxaborolan-2-yl)pyridine (115 mg, 560 μmol; CAS-RN:[329214-79-1]) gave 189 mg of the title compound.

LC-MS (Method 2): Rₜ=1.41 min; MS (ESIpos): m/z=668 [M+H]⁺

¹H-NMR (400 MHz, DMSO-d6) δ[ppm]: −0.009 (1.25), 0.007 (0.94), 1.067 (7.82), 1.156 (0.77), 1.174 (1.58), 1.192 (0.76), 1.989 (2.80), 2.333 (0.51), 2.520 (2.30), 2.525 (1.59), 2.675 (0.50), 3.657 (1.29), 3.694 (0.93), 3.703 (0.81), 3.717 (1.39), 3.728 (16.00), 3.942 (1.25), 4.019 (0.58), 4.037 (0.58), 5.442 (2.50), 6.871 (0.57), 6.878 (2.95), 6.884 (1.03), 6.896 (1.07), 6.900 (3.00), 6.908 (0.40), 7.163 (0.57), 7.180 (0.87), 7.197 (0.58), 7.281 (1.13), 7.302 (0.99), 7.353 (0.72), 7.365 (0.75), 7.372 (0.71), 7.386 (0.72), 7.493 (0.45), 7.509 (0.42), 7.592 (0.42), 8.314 (1.00), 8.318 (1.50), 8.326 (1.12), 8.330 (1.48), 8.338 (1.00), 8.342 (0.62), 8.352 (0.75), 8.357 (0.91), 8.362 (0.60), 9.191 (1.39), 9.195 (1.38).

### Intermediate 71

### N-[(4-methoxyphenyl)methyl]-N-({1-[(4-methoxyphenyl)methyl]-1H-benzimidazol-2-yl}methyl)-2-(morpholin-4-yl)-8-(pyridin-4-yl)pyrazolo[1,5-a][1,3,5]triazin-4-amine

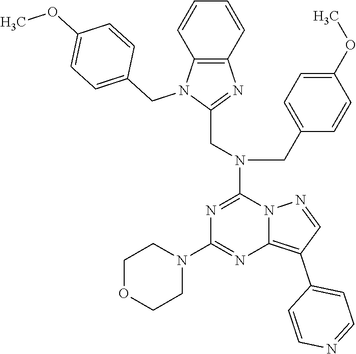 [SMILES: COc1ccc(CN(Cc2nc3ccccc3n2Cc2ccc(OC)cc2)c2nc(N3CCOCC3)nc3c(-c4ccncc4)cnn23)cc1]

In analogy to the procedure described in Intermediate 62 the reaction of 8-bromo-N-[(4-methoxyphenyl)methyl]-N-({1-[(4-methoxyphenyl)methyl]-1H-benzimidazol-2-yl}methyl)-2-(morpholin-4-yl)pyrazolo[1,5-a][1,3,5]triazin-4-amine (Intermediate 61, 250 mg) and 4-(4,4,5,5-tetramethyl-1,3,2-dioxaborolan-2-yl)pyridine (115 mg, 560 μmol; CAS-RN:[181219-01-2]) gave 216 mg of the title compound.

LC-MS (Method 2): Rₜ=1.39 min; MS (ESIpos): m/z=668 [M+H]⁺

¹H-NMR (400 MHz, DMSO-d6) δ[ppm]: −0.008 (1.00), 0.008 (1.05), 1.068 (16.00), 1.156 (2.11), 1.174 (4.20), 1.192 (2.02), 1.235 (0.38), 1.990 (7.57), 2.334 (0.47), 2.338 (0.20), 2.520 (2.35), 2.525 (1.51), 2.676 (0.47), 2.680 (0.20), 3.308 (0.17), 3.545 (0.30), 3.649 (1.00), 3.670 (0.95), 3.694 (0.91), 3.702 (0.77), 3.717 (1.86), 3.727 (15.32), 3.808 (0.30), 3.821 (0.32), 3.940 (3.09), 4.002 (0.56), 4.020 (1.71), 4.037 (1.70), 4.055 (0.55), 4.091 (0.42), 5.440 (2.25), 5.761 (1.19), 6.825 (0.28), 6.847 (0.30), 6.868 (0.47), 6.875 (2.83), 6.880 (0.96), 6.892 (0.99), 6.897 (3.05), 6.904 (0.38), 7.021 (0.27), 7.146 (0.23), 7.164 (0.46), 7.181 (0.74), 7.197 (0.51), 7.215 (0.27), 7.279 (0.95), 7.299 (0.83), 7.335 (0.23), 7.357 (0.18), 7.496 (0.38), 7.514 (0.36), 7.591 (0.34), 7.608 (0.30), 7.946 (2.54), 7.950 (1.51), 7.957 (1.54), 7.961 (2.50), 8.455 (2.76), 8.458 (1.55), 8.467 (1.55), 8.470 (2.32), 8.587 (0.43).

### Intermediate 72

### benzyl [2-(2-amino-3-bromoanilino)-2-oxoethyl]carbamate

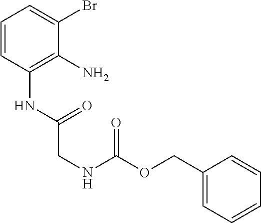 [SMILES: Nc1c(Br)cccc1NC(=O)CNC(=O)OCc1ccccc1]

To a solution of N-[(benzyloxy)carbonyl]glycine (6.15 g, 29.4 mmol, CAS-RN:[1138-80-3]) in dichloromethane (30 mL) were added N,N-diisopropylethylamine (12 mL, 67 mmol; CAS-RN:[7087-68-5]) and propanephosphonic anhydride in ethyl acetate (22.1 g, 50% purity, 34.8 mmol) at 0° C. The reaction solution was stirred at rt for 30 min. 3-bromobenzene-1,2-diamine (5.00 g, 26.7 mmol, CAS-RN:[1575-36-6]) was added to the reaction solution. The reaction mixture was stirred at rt for 12 h. Water was added to the reaction mixture. The organic layer was evaporated under reduced pressure to give a residue. The residue was purified by MPLC (petroleum ether: ethyl acetate=20:1 to 1:1) to give 7.00 g (69% yield) of the title compound as a red oil.

LC-MS (Method D): Rₜ=0.778 min; MS (ESIpos): m/z=378.1 [M+H]⁺.

### Intermediate 73

### benzyl [(4-bromo-1H-benzimidazol-2-yl)methyl]carbamate

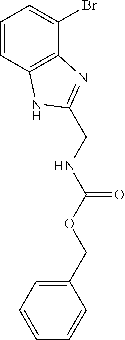 [SMILES: O=C(NCc1nc2c(Br)cccc2[nH]1)OCc1ccccc1]

A solution of benzyl [2-(2-amino-3-bromoanilino)-2-oxoethyl]carbamate (Intermediate 72, 7.00 g, 18.5 mmol) in acetic acid (100 mL) was stirred at 90° C. for 2 h. The reaction mixture was cooled to rt and evaporated under reduced pressure to give a residue. The residue was dissolved in dichloromethane, the solution was washed with saturated aqueous sodium bicarbonate and filtered through silica gel (200-300 mesh). The filtrate was evaporated under reduced pressure to give 5.50 g (83% yield) of the title compound as a yellow oil.

### Intermediate 74

### benzyl [(4-bromo-1-{[2-(trimethylsilyl)ethoxy]methyl}-1H-benzimidazol-2-yl)methyl]carbamate-benzyl [(7-bromo-1-{[2-(trimethylsilyl)ethoxy]methyl}-1H-benzimidazol-2-yl)methyl]carbamate (1/1)

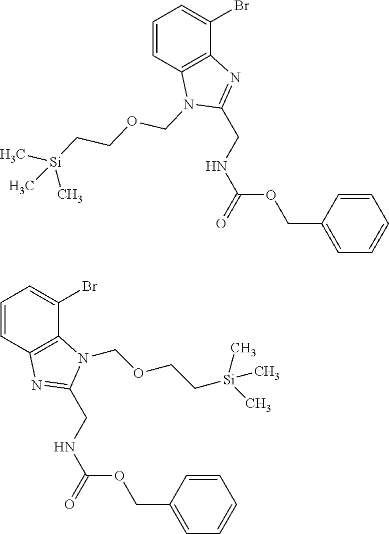 [SMILES: C[Si](C)(C)CCOCn1c(CNC(=O)OCc2ccccc2)nc2c(Br)cccc21 ; C[Si](C)(C)CCOCn1c(CNC(=O)OCc2ccccc2)nc2cccc(Br)c21]

To a solution of benzyl ((4-bromo-1H-benzo[d]imidazol-2-yl)methyl)carbamate (Intermediate 73, 5.50 g, 15.3 mmol) in tetrahydrofuran (55 mL) was added sodium hydride (794 mg, 60% purity, 19.8 mmol) at 0° C. The mixture was warmed to rt and stirred at rt for 0.5 h. (2-(chloromethoxy)ethyl)trimethylsilane (3.05 g, 18.3 mmol, CAS-RN:[76513-69-4]) was added to the mixture at 0° C. The mixture was warmed to rt and stirred at rt for 12 h. Water was added to the reaction mixture. The mixture was extracted with ethyl acetate. The organic layer was evaporated under reduced pressure to give a residue. The residue was purified by chromatography (petroleum ether: ethyl acetate=20:1 to 3:1) to give 4.70 g (97% purity, 61% yield) of the title compound as a yellow solid.

LC-MS (Method D): Rₜ=0.940 min; MS (ESIpos): m/z=490.1 [M+H]⁺.

¹H-NMR (400 MHz, CDCl₃): δ [ppm]=7.47 (m, 2H), 7.35 (m, 5H), 7.32 (m, 1H), 6.00-5.90 (m, 1H), 5.60 (s, 1H), 5.15 (d, 2H), 4.74 (d, 2H), 3.55-3.50 (m, 2H), 0.87 (m, 2H), 0.03-0.05 (m, 9H).

### Intermediate 75

### benzyl [(4-ethenyl-1-{[2-(trimethylsilyl)ethoxy]methyl}-1H-benzimidazol-2-yl)methyl]carbamate-benzyl [(7-ethenyl-1-{[2-(trimethylsilyl)ethoxy]methyl}-1H-benzimidazol-2-yl)methyl]carbamate (1/1)

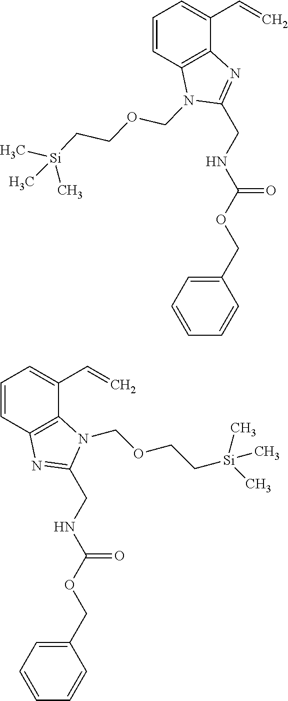 [SMILES: C=Cc1cccc2c1nc(CNC(=O)OCc1ccccc1)n2COCC[Si](C)(C)C ; C=Cc1cccc2nc(CNC(=O)OCc3ccccc3)n(COCC[Si](C)(C)C)c12]

To a solution of benzyl [(4-bromo-1-{[2-(trimethylsilyl)ethoxy]methyl}-1H-benzimidazol-2-yl)methyl]carbamate-benzyl [(7-bromo-1-{[2-(trimethylsilyl)ethoxy]methyl}-1H-benzimidazol-2-yl)methyl]carbamate (1/1) (Intermediate 74, 2.00 g, 97% purity, 3.96 mmol) and potassium vinyltrifluoroborate (1.06 g, 7.91 mmol, CAS-RN:[13682-77-4]) in 1,4-dioxane (32 mL)/water (8.0 mL) were added sodium carbonate (838 mg, 7.91 mmol) and 1,1′-bis(diphenylphosphino)ferrocenepalladium(II) chloride (145 mg, 0.198 mmol, CAS-RN:[72287-26-4]) at rt under nitrogen atmosphere. The reaction mixture was heated to 90° C. and stirred at 90° C. for 12 h under nitrogen atmosphere. The reaction mixture was cooled to rt and filtered. The filtrate was evaporated under reduced pressure to give a residue. The residue was purified by chromatography (200-300 mesh, petroleum ether: ethyl acetate=30:1 to 4:1) to give 1.90 g (55% yield) of the title compound as a yellow oil.

LC-MS (Method D): Rₜ=0.869 min; MS (ESIpos): m/z=438.2 [M+H]⁺.

¹H-NMR (400 MHz, CDCl₃): 5 [ppm]=7.38-7.37 (m, 7H), 7.35-7.34 (m, 1H), 6.36-6.31 (m, 1H), 5.95 (s, 1H), 5.72 (d, 1H), 5.56-5.54 (m, 2H), 5.16 (s, 2H), 4.74 (d, 2H), 3.54-3.50 (m, 2H), 0.96-0.86 (m, 2H), −0.30-0.05 (m, 9H).

### Intermediate 76

### benzyl {[4-(2-hydroxyethyl)-1-{[2-(trimethylsilyl)ethoxy]methyl}-1H-benzimidazol-2-yl]methyl}carbamate-benzyl {[7-(2-hydroxyethyl)-1-{[2-(trimethylsilyl)ethoxy]methyl}-1H-benzimidazol-2-yl]methyl}carbamate (1/1)

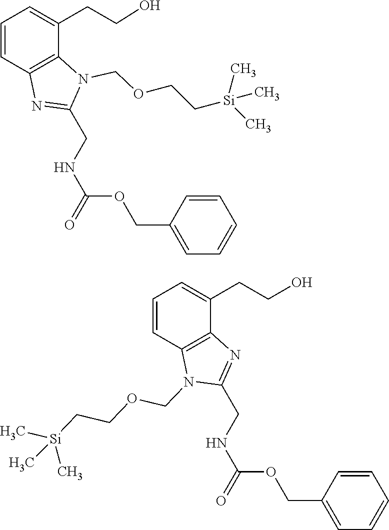

To a solution of benzyl [(4-ethenyl-1-{[2-(trimethylsilyl)ethoxy]methyl}-1H-benzimidazol-2-yl)methyl]carbamate-benzyl [(7-ethenyl-1-{[2-(trimethylsilyl)ethoxy]methyl}-1H-benzimidazol-2-yl)methyl]carbamate (1/1) (Intermediate 75, 4.40 g, 90% purity, 9.05 mmol) in tetrahydrofuran (45 mL, 550 mmol) was added 9-borabicyclo(3.3.1)nonane in tetrahydrofuran (45 mL, 0.50 M, 23 mmol; CAS-RN:[280-64-8]) at rt. The reaction mixture was stirred at rt for 12 h. Ethanol (45 mL) was added to the reaction mixture, then sodium hydroxide (17 mL, 1.0 M, 17 mmol) and hydrogen peroxide (17 mL, 30% purity, 170 mmol) were added to the mixture at rt. The reaction mixture was stirred at rt for 12 h. Saturated sodium thiosulfate was added to the reaction mixture. The mixture was extracted with ethyl acetate, the organic layer was washed with glycerol/water (1/100) and then evaporated under reduced pressure to give a residue. The residue was purified by chromatography (1000 mesh, petroleum ether: ethyl acetate=5:1 to 1:1) to give 3.20 g (95% purity, 74% yield) of the title compound as a light yellow oil.

LC-MS (Method D): Rₜ=0.844 min; MS (ESIpos): m/z=456.3 [M+H]⁺.

### Intermediate 77

### 2-[2-({[(benzyloxy)carbonyl]amino}methyl)-1-{[2-(trimethylsilyl)ethoxy]methyl}-1H-benzimidazol-4-yl]ethyl methanesulfonate-2-[2-({[(benzyloxy)carbonyl]amino}methyl)-1-{[2-(trimethylsilyl)ethoxy]methyl}-1H-benzimidazol-7-yl]ethyl methanesulfonate (1/1)

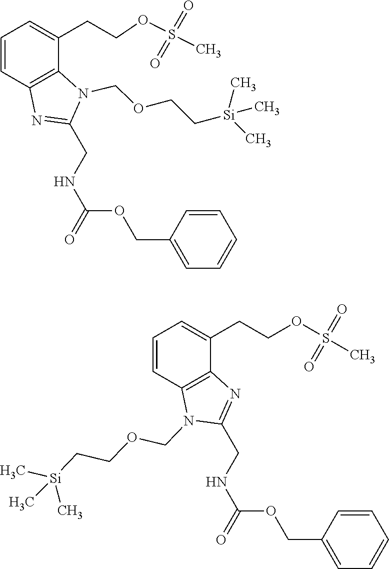

To a solution of benzyl {[4-(2-hydroxyethyl)-1-{[2-(trimethylsilyl)ethoxy]methyl}-1H-benzimidazol-2-yl]methyl}carbamate-benzyl {[7-(2-hydroxyethyl)-1-{[2-(trimethylsilyl)ethoxy]methyl}-1H-benzimidazol-2-yl]methyl}carbamate (1/1) (Intermediate 76, 3.00 g, 95% purity, 6.26 mmol) and methanesulfonyl chloride (860 mg, 7.51 mmol) in dichloromethane (30 mL, 460 mmol) was added trimethylamine (2.6 mL, 19 mmol; CAS-RN:[121-44-8]) at 0° C. The reaction was warmed to rt and stirred at room temperature for 1 h. The reaction mixture was evaporated under reduced pressure to give a residue. The residue was dissolved in ethyl acetate, the solution was filtered, the filtrate was evaporated under reduced pressure to give 4.20 g (75% purity, 94% yield) of the title compound as a yellow oil.

LC-MS (Method D): Rₜ=0.899 min; MS (ESIpos): m/z=534.3 [M+H]⁺.

### Intermediate 78

### benzyl {[4-(2-azidoethyl)-1-{[2-(trimethylsilyl)ethoxy]methyl}-1H-benzimidazol-2-yl]methyl}carbamate-benzyl {[7-(2-azidoethyl)-1-{[2-(trimethylsilyl)ethoxy]methyl}-1H-benzimidazol-2-yl]methyl}carbamate (1/1)

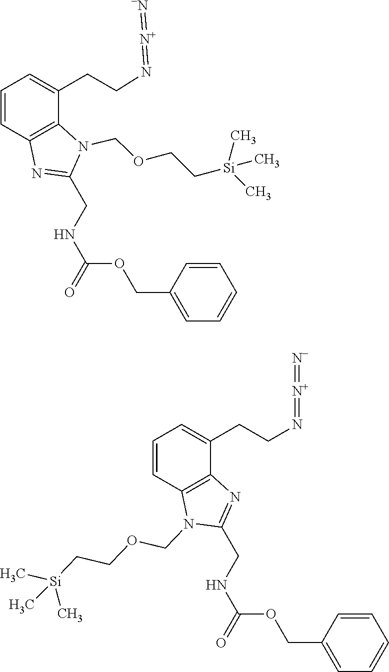

To a solution of 2-[2-({[(benzyloxy)carbonyl]amino}methyl)-1-{[2-(trimethylsilyl)ethoxy]methyl}-1H-benzimidazol-4-yl]ethyl methanesulfonate-2-[2-({[(benzyloxy)carbonyl]amino}methyl)-1-{[2-(trimethylsilyl)ethoxy]methyl}-1H-benzimidazol-7-yl]ethyl methanesulfonate (1/1) (Intermediate 77, 4.20 g, 75% purity, 5.90 mmol) in DMF (40 mL) was added sodium azide (537 mg, 8.26 mmol, CAS-RN:[26628-22-8]) at rt. The reaction mixture was heated to 70° C. and stirred at 70° C. for 12 h. The reaction mixture was cooled to rt. Water was added to the reaction mixture, the solution was extracted with ethyl acetate. The organic layer was evaporated to remove 3/4 volume of ethyl acetate and diluted with tetrahydrofuran to give 3.20 g (85% purity, 96% yield) of the title compound in tetrahydrofuran.

### Intermediate 79

### benzyl {[4-(2-aminoethyl)-1-{[2-(trimethylsilyl)ethoxy]methyl}-1H-benzimidazol-2-yl]methyl}carbamate-benzyl {[7-(2-aminoethyl)-1-{[2-(trimethylsilyl)ethoxy]methyl}-1H-benzimidazol-2-yl]methyl}carbamate (1/1)

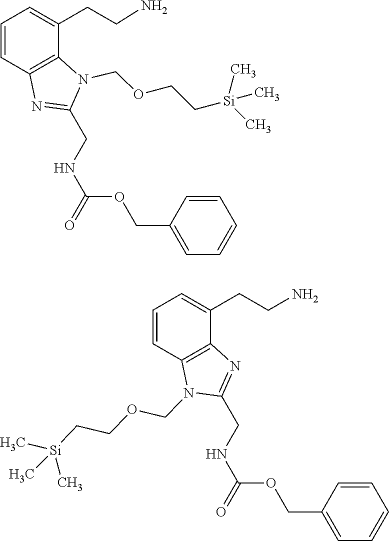

To a solution of benzyl {[4-(2-azidoethyl)-1-{[2-(trimethylsilyl)ethoxy]methyl}-1H-benzimidazol-2-yl]methyl}carbamate-benzyl {[7-(2-azidoethyl)-1-{[2-(trimethylsilyl)ethoxy]methyl}-1H-benzimidazol-2-yl]methyl}carbamate (1/1) (Intermediate 78, 3.20 g, 85% purity, 5.66 mmol) in THF (40 mL) were added water and triphenylphosphine (1.78 g, 6.79 mmol; CAS-RN:[603-35-0]) at rt. The reaction mixture was heated to 50° C. and stirred at 50° C. for 12 h. The reaction mixture was cooled to rt. Water was added to the reaction mixture. The solution was adjusted to pH 4 by hydrochloric acid (1 M in water) and then washed with ethyl acetate. The organic layer was adjusted to pH 8-9 by saturated sodium bicarbonate and then extracted with ethyl acetate. The organic layer was evaporated under reduced pressure to give 2.20 g (71% purity, 61% yield) of the title compound as a yellow oil

LC-MS (Method D): Rₜ=0.800 min; MS (ESIpos): m/z=455.4 [M+H]⁺.

### Intermediate 80

### benzyl [(4-{2-[(tert-butoxycarbonyl)amino]ethyl}-1-{[2-(trimethylsilyl)ethoxy]methyl}-1H-benzimidazol-2-yl)methyl]carbamate-benzyl [(7-{2-[(tert-butoxycarbonyl)amino]ethyl}-1-{[2-(trimethylsilyl)ethoxy]methyl}-1H-benzimidazol-2-yl)methyl]carbamate (1/1)

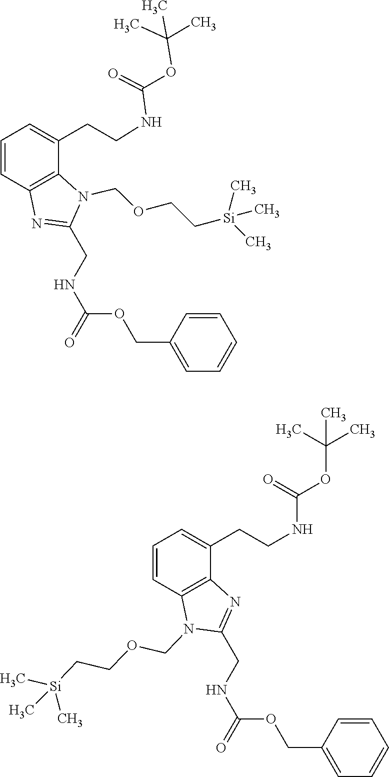

To a solution of benzyl {[4-(2-aminoethyl)-1-{[2-(trimethylsilyl)ethoxy]methyl}-1H-benzimidazol-2-yl]methyl}carbamate-benzyl {[7-(2-aminoethyl)-1-{[2-(trimethylsilyl)ethoxy]methyl}-1H-benzimidazol-2-yl]methyl}carbamate (1/1) (Intermediate 79, 2.20 g, 71% purity, 3.44 mmol) in THF (20 mL) was added di-tert-butyl dicarbonate (950 μL, 4.1 mmol; CAS-RN:[24424-99-5]) at rt. The reaction mixture was stirred at rt for 12 h. Water was added to the reaction mixture, the solution was extracted with ethyl acetate. The organic layer was evaporated under reduced pressure to give a residue. The residue was purified by flash chromatography (200-300 mesh, petroleum ether:ethyl acetate=1: 0-1:1) to give 2.30 g (76% purity, 92% yield) of the title compound as a colorless oil LC-MS (Method D): Rₜ=0.931 min; MS (ESIpos): m/z=555.4 [M+H]⁺.

### Intermediate 81

### tert-butyl {2-[2-(aminomethyl)-1-{[2-(trimethylsilyl)ethoxy]methyl}-1H-benzimidazol-4-yl]ethyl}carbamate-tert-butyl {2-[2-(aminomethyl)-1-{[2-(trimethylsilyl)ethoxy]methyl}-1H-benzimidazol-7-yl]ethyl}carbamate (1/1)

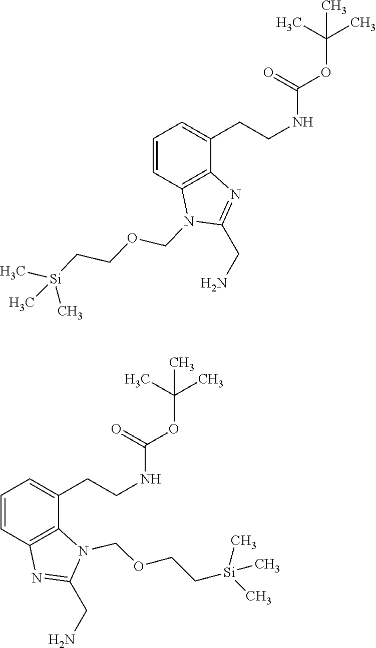 [SMILES: CC(C)(C)OC(=O)NCCc1cccc2c1nc(CN)n2COCC[Si](C)(C)C ; CC(C)(C)OC(=O)NCCc1cccc2nc(CN)n(COCC[Si](C)(C)C)c12]

To a solution of benzyl [(4-{2-[(tert-butoxycarbonyl)amino]ethyl}-1-{[2-(trimethylsilyl)ethoxy]methyl}-1H-benzimidazol-2-yl)methyl]carbamate-benzyl [(7-{2-[(tert-butoxycarbonyl)amino]ethyl}-1-{[2-(trimethylsilyl)ethoxy]methyl}-1H-benzimidazol-2-yl)methyl]carbamate (1/1) (Intermediate 80, 2.10 g, 76% purity, 2.88 mmol) in methanol (30 mL) was added palladium on carbon (1.00 g, 10% purity) at rt. The reaction mixture was stirred at rt under hydrogen atmosphere. The reaction mixture was filtered through celite, the filtrate was evaporated under reduced pressure to give 1.50 g (73% purity, 90% yield) of the title compound as a colorless oil.

LC-MS (Method D): Rₜ=0.826 min; MS (ESIpos): m/z=421.4 [M+H]⁺.

### Intermediate 82

### tert-butyl {2-[2-({[8-bromo-2-(methylsulfanyl)pyrazolo[1,5-a][1,3,5]triazin-4-yl]amino}methyl)-1-{[2-(trimethylsilyl)ethoxy]methyl}-1H-benzimidazol-4-yl]ethyl}carbamate-tert-butyl {2-[2-({[8-bromo-2-(methylsulfanyl)pyrazolo[1,5-a][1,3,5]triazin-4-yl]amino}methyl)-1-{[2-(trimethylsilyl)ethoxy]methyl}-1H-benzimidazol-7-yl]ethyl}carbamate (1/1)

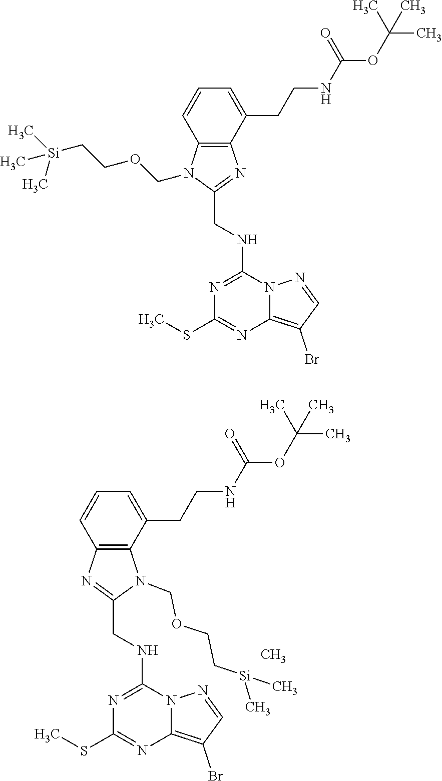

To a solution of tert-butyl {2-[2-(aminomethyl)-1-{[2-(trimethylsilyl)ethoxy]methyl}-1H-benzimidazol-4-yl]ethyl}carbamate-tert-butyl {2-[2-(aminomethyl)-1-{[2-(trimethylsilyl)ethoxy]methyl}-1H-benzimidazol-7-yl]ethyl}carbamate (1:1) (Intermediate 81, 1.50 g, 73% purity, 2.60 mmol) in 1- butanol were added 8-bromo-4-chloro-2-(methylsulfanyl)pyrazolo[1,5-a][1,3,5]triazine (Intermediate 7, 1.23 g, 89% purity, 3.90 mmol) and N,N-diisopropylethylamine (1.4 mL, 7.8 mmol) at rt. The reaction mixture was heated to 80° C. and stirred at 80° C. for 12 h. The reaction mixture was cooled to rt and evaporated under reduced pressure to give a residue. The residue was dissolved in ethyl acetate, the solution was washed with water. The organic layer was evaporated under reduced pressure to give a residue. The residue was purified by chromatography (petroleum ether: ethyl acetate=20:1 to 1:1) to give 1.90 g (90% purity, 99% yield) as a yellow oil.

LC-MS (Method D): Rₜ=0.991 min; MS (ESIpos): m/z=663.3 [M+H]⁺.

### Intermediate 83

### tert-butyl {2-[2-({[8-bromo-2-(methanesulfonyl)pyrazolo[1,5-a][1,3,5]triazin-4-yl]amino}methyl)-1-{[2-(trimethylsilyl)ethoxy]methyl}-1H-benzimidazol-4-yl]ethyl}carbamate-tert-butyl {2-[2-({[8-bromo-2-(methanesulfonyl)pyrazolo[1,5-a][1,3,5]triazin-4-yl]amino}methyl)-1-{[2-(trimethylsilyl)ethoxy]methyl}-1H-benzimidazol-7-yl]ethyl}carbamate (1/1)

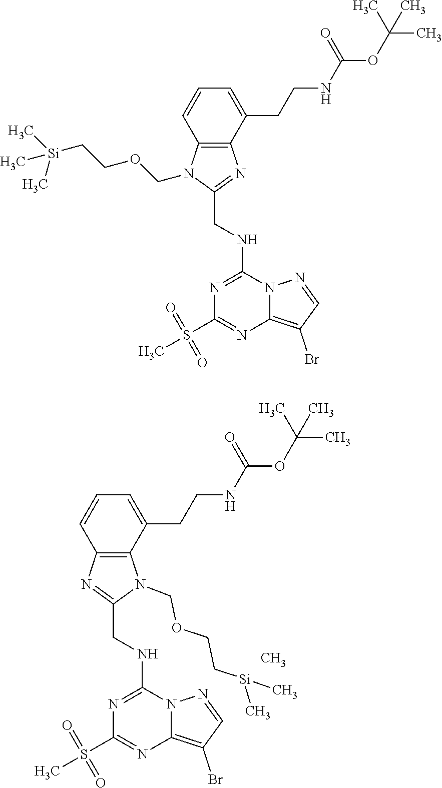

To a solution of tert-butyl {2-[2-({[8-bromo-2-(methylsulfanyl)pyrazolo[1,5-a][1,3,5]triazin-4-yl]amino}methyl)-1-{[2-(trimethylsilyl)ethoxy]methyl}-1H-benzimidazol-4-yl]ethyl}carbamate-tert-butyl {2-[2-({[8-bromo-2-(methylsulfanyl)pyrazolo[1,5-a][1,3,5]triazin-4-yl]amino}methyl)-1-{[2-(trimethylsilyl)ethoxy]methyl}-1H-benzimidazol-7-yl]ethyl}carbamate (1:1) (Intermediate 82, 1.90 g, 90% purity, 2.58 mmol) in dichloromethane (30 mL) was added meta-chloroperoxybenzoic acid (1.33 g, 7.73 mmol, CAS-RN:[937-14-4]) at rt. The reaction mixture was stirred at rt for 12 h. Water was added to the reaction mixture. The solution was extracted with ethyl acetate. The organic layer was evaporated under reduced pressure to give 3.10 g (49% purity, 85% yield) as a yellow oil.

LC-MS (Method D): Rₜ=0.877 min; MS (ESIpos): m/z=695.1 [M+H]⁺.

### Intermediate 84

### tert-butyl {2-[2-({[8-bromo-2-(morpholin-4-yl)pyrazolo[1,5-a][1,3,5]triazin-4-yl]amino}methyl)-1-{[2-(trimethylsilyl)ethoxy]methyl}-1H-benzimidazol-4-yl]ethyl}carbamate-tert-butyl {2-[2-({[8-bromo-2-(morpholin-4-yl)pyrazolo[1,5-a][1,3,5]triazin-4-yl]amino}methyl)-1-{[2-(trimethylsilyl)ethoxy]methyl}-1H-benzimidazol-7-yl]ethyl}carbamate (1/1)

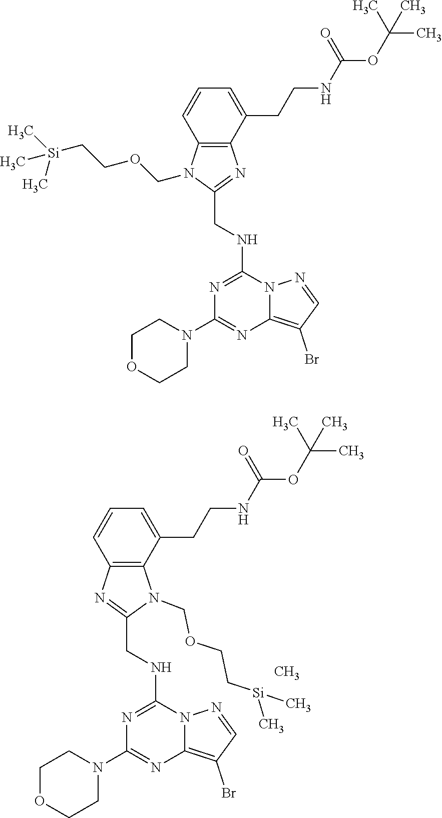

To a solution of tert-butyl {2-[2-({[8-bromo-2-(methylsulfinyl)pyrazolo[1,5-a][1,3,5]triazin-4-yl]amino}methyl)-1-{[2-(trimethylsilyl)ethoxy]methyl}-1H-benzimidazol-4-yl]ethyl}carbamate-tert-butyl {2-[2-({[8-bromo-2-(methylsulfinyl)pyrazolo[1,5-a][1,3,5]triazin-4-yl]amino}methyl)-1-{[2-(trimethylsilyl)ethoxy]methyl}-1H-benzimidazol-7-yl]ethyl}carbamate (1:1) (Intermediate 83, 270 mg, 48% purity, 0.191 mmol) in acetonitrile (10 mL) were added morpholine (49.8 mg, 0.572 mmol, CAS-RN:[110-91-8]) and N,N-diisopropylethylamine (0.100 mL, 0.570 mmol) at rt. The reaction mixture was heated to 80° C. and stirred at 80° C. for 12 h. The reaction mixture was evaporated under reduced pressure to give a residue. The residue was purified by preparative-TLC (dichloromethane:methanol=10:1) to give 100 mg (78% purity, 58% yield) as a yellow oil.

LC-MS (Method D): Rₜ=0.895 min; MS (ESIpos): m/z=702.2 [M+H]⁺.

### Intermediate 85

### N-{[7-(2-aminoethyl)-1H-benzimidazol-2-yl]methyl}-8-bromo-2-(morpholin-4-yl)pyrazolo[1,5-a][1,3,5]triazin-4-amine-N-{[7-(2-aminoethyl)-1H-benzimidazol-2-yl]methyl}-8-chloro-2-(morpholin-4-yl)pyrazolo[1,5-a][1,3,5]triazin-4-amine (1/1)

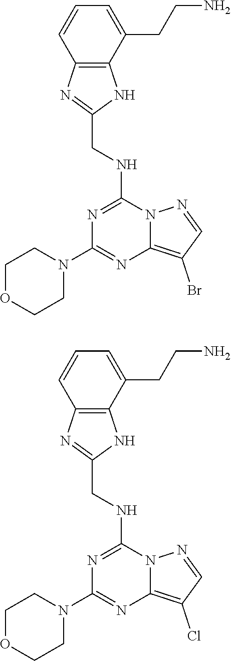 [SMILES: NCCc1cccc2nc(CNc3nc(N4CCOCC4)nc4c(Br)cnn34)[nH]c12 ; NCCc1cccc2nc(CNc3nc(N4CCOCC4)nc4c(Cl)cnn34)[nH]c12]

A solution of tert-butyl {2-[2-({[8-bromo-2-(morpholin-4-yl)pyrazolo[1,5-a][1,3,5]triazin-4-yl]amino}methyl)-1-{[2-(trimethylsilyl)ethoxy]methyl}-1H-benzimidazol-4-yl]ethyl}carbamate-tert-butyl {2-[2-({[8-bromo-2-(morpholin-4-yl)pyrazolo[1,5-a][1,3,5]triazin-4-yl]amino}methyl)-1-{[2-(trimethylsilyl)ethoxy]methyl}-1H-benzimidazol-7-yl]ethyl}carbamate (1/1) (Intermediate 84, 1.90 g, 78% purity, 2.11 mmol) in hydrochloric acid (4 M in ethyl acetate, 30 mL) was stirred at rt for 12 h. The reaction mixture was evaporated under reduced pressure to give a residue. The residue was purified by preparative-HPLC (Instrument: Shimadzu LC-20AP; Column: Phenomenex luna C18 250*50 mm*10 um; eluent A: water (0.05% HCl), eluent B: acetonitrile; gradient: 0-30 min 0-30% B; flow 100 mL/min; temperature: RT; Detector: UV 220/254 nm.) to give 510 mg (54% yield) of the title compound as a yellow oil.

### Intermediate 86

### benzyl [(4-{3-[(tert-butoxycarbonyl)amino]prop-1-yn-1-yl}-1-{[2-(trimethylsilyl)ethoxy]methyl}-1H-benzimidazol-2-yl)methyl]carbamate-benzyl [(7-{3-[(tert-butoxycarbonyl)amino]prop-1-yn-1-yl}-1-{[2-(trimethylsilyl)ethoxy]methyl}-1H-benzimidazol-2-yl)methyl]carbamate (1/1)

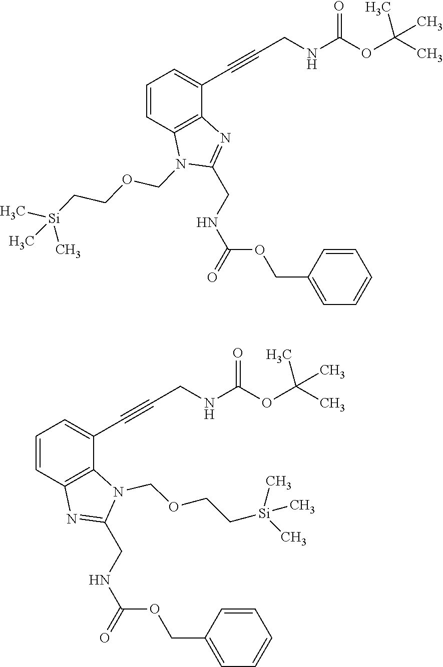 [SMILES: CC(C)(C)OC(=O)NCC#Cc1cccc2c1nc(CNC(=O)OCc1ccccc1)n2COCC[Si](C)(C)C ; CC(C)(C)OC(=O)NCC#Cc1cccc2nc(CNC(=O)OCc3ccccc3)n(COCC[Si](C)(C)C)c12]

To a solution of benzyl [(4-bromo-1-{[2-(trimethylsilyl)ethoxy]methyl}-1H-benzimidazol-2-yl)methyl]carbamate-benzyl [(7-bromo-1-{[2-(trimethylsilyl)ethoxy]methyl}-1H-benzimidazol-2-yl)methyl]carbamate (1/1) (Intermediate 74, 5.00 g, 95% purity, 9.68 mmol) and tert-butyl prop-2-yn-1-ylcarbamate (4.51 g, 29.1 mmol, CAS-RN:[262418-92-8]) in acetonitrile (48 mL) were added cesium carbonate (9.47 g, 29.1 mmol; CAS-RN:[534-17-8]) and chloro[(di(1-adamantyl)-N-butylphosphine)-2-(2-aminobiphenyl)]palladium(II) (475 mg; CAS-RN:[1375477-29-4]). The reaction mixture was stirred at 70° C. for 1 h. The reaction was concentrated in vacuum, purified by silica gel chromatography (petroleum ether: ethyl acetate=10:1 to 2:1) to give 6.40 g (88% purity, 51% yield) of the title compound as a red gum.

LC-MS (Method D): Rₜ=0.899 min; MS (ESIpos): m/z=565.2 [M+H]⁺.

### Intermediate 87

### tert-butyl {3-[2-(aminomethyl)-1-{[2-(trimethylsilyl)ethoxy]methyl}-1H-benzimidazol-4-yl]propyl}carbamate-tert-butyl {3-[2-(aminomethyl)-1-{[2-(trimethylsilyl)ethoxy]methyl}-1H-benzimidazol-7-yl]propyl}carbamate (1/1)

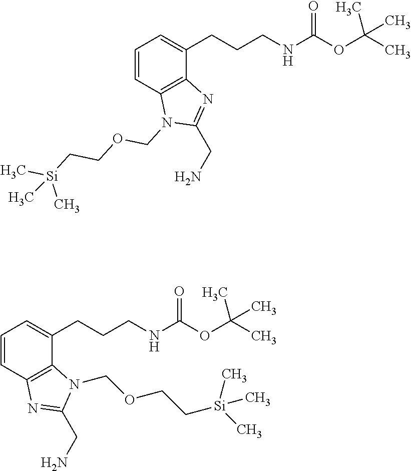 [SMILES: CC(C)(C)OC(=O)NCCCc1cccc2c1nc(CN)n2COCC[Si](C)(C)C ; CC(C)(C)OC(=O)NCCCc1cccc2nc(CN)n(COCC[Si](C)(C)C)c12]

To a solution of benzyl [(4-{3-[(tert-butoxycarbonyl)amino]prop-1-yn-1-yl}-1-{[2-(trimethylsilyl)ethoxy]methyl}-1H-benzimidazol-2-yl)methyl]carbamate-benzyl [(7-{3-[(tert-butoxycarbonyl)amino]prop-1-yn-1-yl}-1-{[2-(trimethylsilyl)ethoxy]methyl}-1H-benzimidazol-2-yl)methyl]carbamate (1:1) (Intermediate 86, 6.40 g, 88% purity, 9.97 mmol) in methanol (100 mL) was added palladium (10% on carbon) (1.00 g, 10% purity) under hydrogen atmosphere. After stirring at rt for 2 h, the reaction mixture was filtered. The filtrate was concentrated and purified by preparative HPLC [Gilson-281; Column: Kromasil Eternity XT250*80 mm*10 μm; eluent A: water (0.05% ammonia hydroxide v/v), eluent B: acetonitrile; gradient: 0-20 min, 50%-80% B; flow 140 mL/min; temperature: RT; Detector: UV 220/254 nm] to give 1.05 g (97% purity, 23% yield) of the title compound as a yellow gum.

LC-MS (Method D): Rₜ=0.770 min; MS (ESIpos): m/z=435.2 [M+H]⁺.

¹H-NMR (400 MHz, DMSO-d₆): δ [ppm]=7.44-7.41 (m, 1H), 7.19-7.07 (m, 1H), 7.00-6.96 (m, 1H), 6.96-6.94 (m, 1H), 5.63-5.60 (m, 2H), 4.00-3.99 (m, 2H), 3.55-3.50 (m, 2H), 3.02-2.89 (m, 4H), 2.06 (s, 2H), 1.79-1.70 (m, 2H), 1.37 (s, 9H), 0.88-0.81 (m, 2H), −0.06-0.10 (m, 9H).

### Intermediate 88

### tert-butyl {3-[2-({[8-bromo-2-(methylsulfanyl)pyrazolo[1,5-a][1,3,5]triazin-4-yl]amino}methyl)-1-{[2-(trimethylsilyl)ethoxy]methyl}-1H-benzimidazol-4-yl]propyl}carbamate-tert-butyl {3-[2-({[8-bromo-2-(methylsulfanyl)pyrazolo[1,5-a][1,3,5]triazin-4-yl]amino}methyl)-1-{[2-(trimethylsilyl)ethoxy]methyl}-1H-benzimidazol-7-yl]propyl}carbamate (1/1)

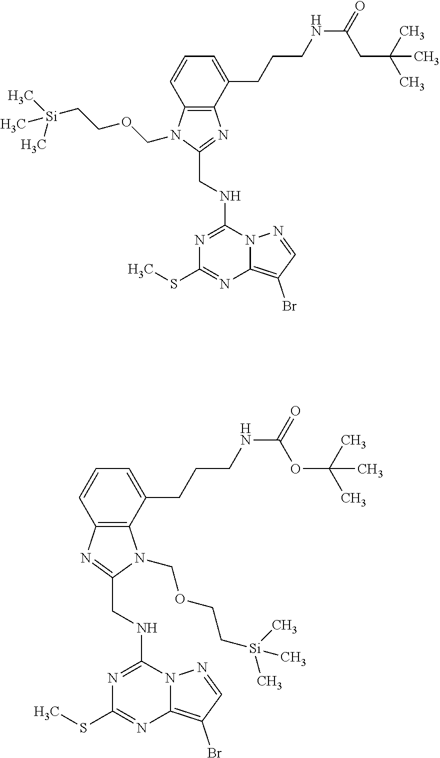

Tert-butyl {3-[2-(aminomethyl)-1-{[2-(trimethylsilyl)ethoxy]methyl}-1H-benzimidazol-4-yl]propyl}carbamate-tert-butyl {3-[2-(aminomethyl)-1-{[2-(trimethylsilyl)ethoxy]methyl}-1H-benzimidazol-7-yl]propyl}carbamate (1/1) (Intermediate 87, 1.20 g, 97% purity, 2.68 mmol) in 1- butanol (12 mL) were added 8-bromo-4-chloro-2-(methylsulfanyl)pyrazolo[1,5-a][1,3,5]triazine (Intermediate 7, 1.12 g, 4.02 mmol) and N,N-diisopropylethylamine (1.4 mL, 8.0 mmol; CAS-RN:[7087-68-5]). The reaction mixture was stirred at 90° C. for 16 h. The reaction was concentrated and purified by silica gel chromatography (petroleum ether: ethyl acetate=10:1 to 1:1) to give 1.30 g (93% purity, 67% yield) of the title compound as a brown oil.

LC-MS (Method D): Rₜ=0.927 min; MS (ESIpos): m/z=677.2 [M+H]⁺.

### Intermediate 89

### tert-butyl {3-[2-({[8-bromo-2-(methanesulfonyl)pyrazolo[1,5-a][1,3,5]triazin-4-yl]amino}methyl)-1-{[2-(trimethylsilyl)ethoxy]methyl}-1H-benzimidazol-4-yl]propyl}carbamate-tert-butyl {3-[2-({[8-bromo-2-(methanesulfonyl)pyrazolo[1,5-a][1,3,5]triazin-4-yl]amino}methyl)-1-{[2-(trimethylsilyl)ethoxy]methyl}-1H-benzimidazol-7-yl]propyl}carbamate (1/1)

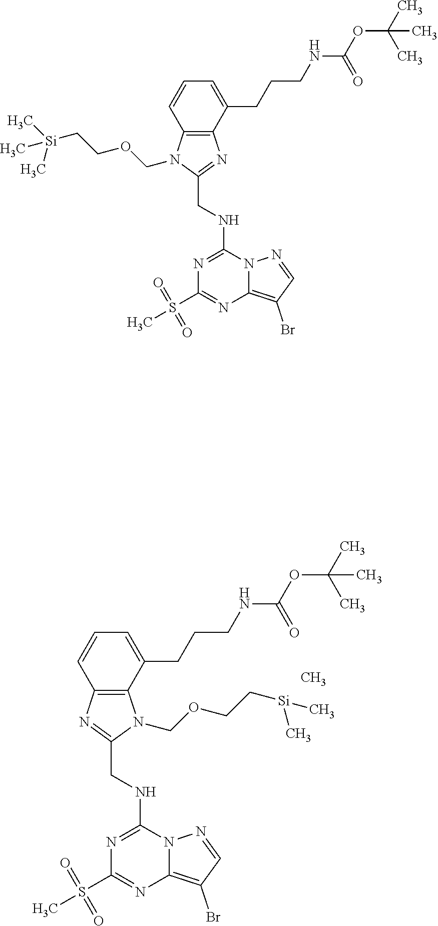

To a solution of tert-butyl {3-[2-({[8-bromo-2-(methylsulfanyl)pyrazolo[1,5-a][1,3,5]triazin-4-yl]amino}methyl)-1-{[2-(trimethylsilyl)ethoxy]methyl}-1H-benzimidazol-4-yl]propyl}carbamate-tert-butyl {3-[2-({[8-bromo-2-(methylsulfanyl)pyrazolo[1,5-a][1,3,5]triazin-4-yl]amino}methyl)-1-{[2-(trimethylsilyl)ethoxy]methyl}-1H-benzimidazol-7-yl]propyl}carbamate (1:1) (Intermediate 88, 1.30 g, 93% purity, 0.892 mmol) in dichloromethane (12 mL) was added meta-chloroperoxybenzoic acid (462 mg, 2.68 mmol, CAS-RN:[937-14-4]). After stirring at rt for 2 h, the reaction mixture was poured into water. The solution was extracted with ethyl acetate. The organic layer was dried over anhydrous sodium sulfate, filtered and the filtrate was concentrated in vacuum to give 1.90 g (63% purity, 95% yield) of the title compound as a yellow oil.

### Intermediate 90

### tert-butyl {3-[2-({[8-bromo-2-(morpholin-4-yl)pyrazolo[1,5-a][1,3,5]triazin-4-yl]amino}methyl)-1-{[2-(trimethylsilyl)ethoxy]methyl}-1H-benzimidazol-4-yl]propyl}carbamate-tert-butyl {3-[2-({[8-bromo-2-(morpholin-4-yl)pyrazolo[1,5-a][1,3,5]triazin-4-yl]amino}methyl)-1-{[2-(trimethylsilyl)ethoxy]methyl}-1H-benzimidazol-7-yl]propyl}carbamate (1/1)

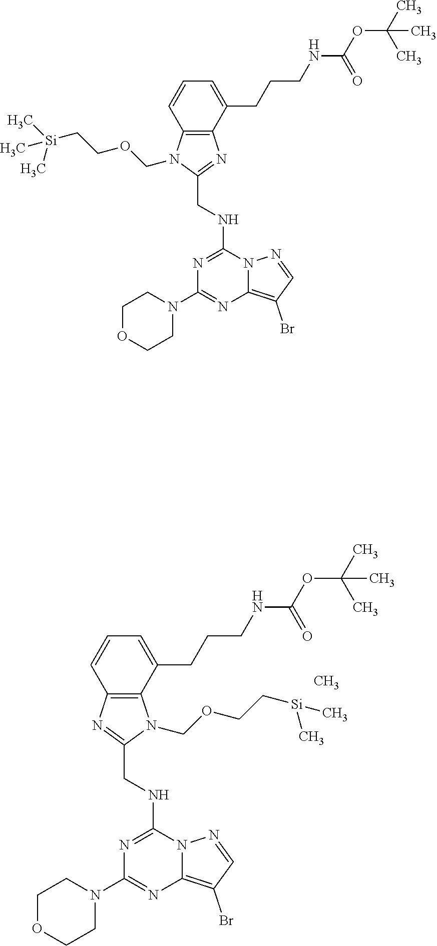

To a solution of tert-butyl {3-[2-({[8-bromo-2-(methanesulfonyl)pyrazolo[1,5-a][1,3,5]triazin-4-yl]amino}methyl)-1-{[2-(trimethylsilyl)ethoxy]methyl}-1H-benzimidazol-4-yl]propyl}carbamate-tert-butyl {3-[2-({[8-bromo-2-(methanesulfonyl)pyrazolo[1,5-a][1,3,5]triazin-4-yl]amino}methyl)-1-{[2-(trimethylsilyl)ethoxy]methyl}-1H-benzimidazol-7-yl]propyl}carbamate (1/1) (Intermediate 89, 1.90 g, 63% purity, 843 μmol) in acetonitrile (16 mL) were added N,N-diisopropylethylamine (440 μL, 2.5 mmol; CAS-RN:[7087-68-5]) and morpholine (220 μL, 2.5 mmol; CAS-RN:[110-91-8]). The reaction mixture was stirred at 80° C. for 3 h. The reaction was concentrated and purified by silica gel chromatography (200-300 mesh, petroleum ether: ethyl acetate=10:1 to 0:1) to give 1.30 g (94% purity, 101% yield) of the title compound.

LC-MS (Method D): Rₜ=0.898 min; MS (ESIpos): m/z=716.2 [M+H]⁺.

### Intermediate 91

### benzyl [2-(2-amino-4-bromoanilino)-2-oxoethyl]carbamate

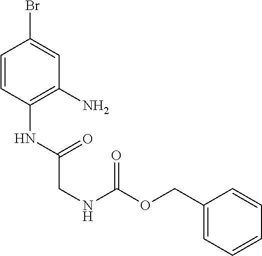 [SMILES: Nc1cc(Br)ccc1NC(=O)CNC(=O)OCc1ccccc1]

To a solution of N-[(benzyloxy)carbonyl]glycine (22.4 g, 107 mmol, CAS-RN:[1138-80-3]) in dichloromethane (300 mL) were added 1-hydroxybenzotriazole (19.7 g, 128 mmol, CAS-RN:[2592-95-2]), 1-(3-dimethylaminopropyl)-3-ethylcarbodiimide hydrochloride (24.6 g, 128 mmol, CAS-RN:[25952-53-8]) and N,N-diisopropylethylamine (37 mL, 210 mmol) at 0° C. The mixture was stirred at rt for 1 h. A solution of 4-bromobenzene-1,2-diamine (20.0 g, 107 mmol, CAS-RN:[1575-37-7]) in dichloromethane (100 mL) was added dropwise to above mixture and the mixture was stirred at rt for 16 h. The reaction mixture was diluted with dichloromethane and washed with brine. The organic phase was dried over anhydrous sodium sulfate, filtered and concentrated to give a residue. The residue was purified by column chromatography (1000 mesh, petroleum ether: ethyl acetate=20:1 to 1:1) to give 12.0 g (80% purity, 24% yield) of the title compound as a yellow solid.

LC-MS (Method C): Rₜ=0.741 min; MS (ESIpos): m/z=378.0/380.0 [M+H]⁺.

### Intermediate 92

### benzyl [(5-bromo-1H-benzimidazol-2-yl)methyl]carbamate

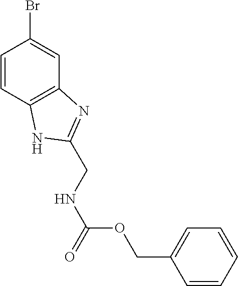 [SMILES: O=C(NCc1nc2cc(Br)ccc2[nH]1)OCc1ccccc1]

Benzyl [2-(2-amino-4-bromoanilino)-2-oxoethyl]carbamate (Intermediate 91, 12.0 g, 80% purity, 25.4 mmol) was dissolved in acetic acid (120 mL). After stirring at 100° C. for 2 h, the reaction mixture was concentrated under reduced pressure, diluted with saturated sodium bicarbonate solution and extracted with ethyl acetate. The combined organic layers were dried over anhydrous sodium sulfate, filtered and concentrated under reduced pressure to give a residue. The residue was triturated with petroleum ether: ethyl acetate (1/1, 50 mL) to afford 9.50 g (95% purity, 99% yield) of the title compound as a light yellow solid.

LC-MS (Method C): Rₜ=0.677 min; MS (ESIpos): m/z=360.0/362.0 [M+H]⁺.

### Intermediate 93

### benzyl [(5-bromo-1-{[2-(trimethylsilyl)ethoxy]methyl}-1H-benzimidazol-2-yl)methyl]carbamate-benzyl [(6-bromo-1-{[2-(trimethylsilyl)ethoxy]methyl}-1H-benzimidazol-2-yl)methyl]carbamate (1/1)

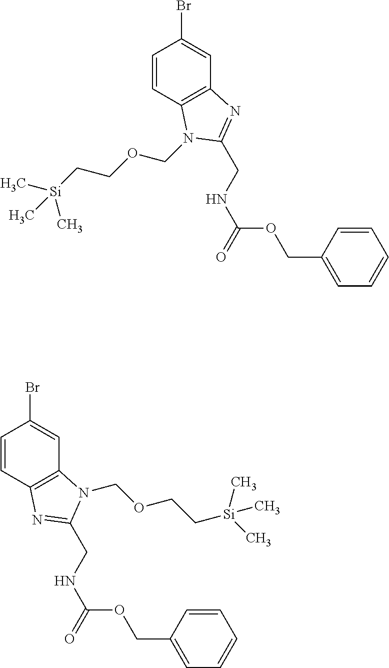 [SMILES: C[Si](C)(C)CCOCn1c(CNC(=O)OCc2ccccc2)nc2cc(Br)ccc21 ; C[Si](C)(C)CCOCn1c(CNC(=O)OCc2ccccc2)nc2ccc(Br)cc21]

To a solution of benzyl [(5-bromo-1H-benzimidazol-2-yl)methyl]carbamate (Intermediate 92, 8.25 g, 95% purity, 21.8 mmol) in tetrahydrofuran (160 mL) was added sodium hydride (1.04 g, 60% purity, 26.1 mmol) at 0° C. in portions. The reaction mixture was stirred at 25° C. for 0.5 h. Then [2-(chloromethoxy)ethyl](trimethyl)silane (3.99 g, 23.9 mmol, CAS-RN:[76513-69-4]) was added dropwise at 0° C. After stirring at 25° C. for 4 h, the reaction mixture was poured into ice-water, and extracted with ethyl acetate. The combined organic layers were washed with brine, dried over anhydrous sodium sulfate, filtered and concentrated under reduced pressure to give a residue. The residue was purified by column chromatography (1000 mesh, petroleum ether: ethyl acetate=20:1 to 10:1) to give 9.50 g (89% yield) of the title compound as a yellow oil.

¹H-NMR (400 MHz, DMSO-d₆): 5 [ppm]=7.99-7.79 (m, 2H), 7.65-7.54 (m, 1H), 7.45-7.27 (m, 6H), 5.64 (s, 2H), 5.06 (s, 2H), 4.55 (t, 2H), 3.50 (t, 2H), 0.82 (t, 2H), −0.09 (s, 9H).

### Intermediate 94

### benzyl [(5-ethenyl-1-{[2-(trimethylsilyl)ethoxy]methyl}-1H-benzimidazol-2-yl)methyl]carbamate-benzyl [(6-ethenyl-1-{[2-(trimethylsilyl)ethoxy]methyl}-1H-benzimidazol-2-yl)methyl]carbamate (1/1)

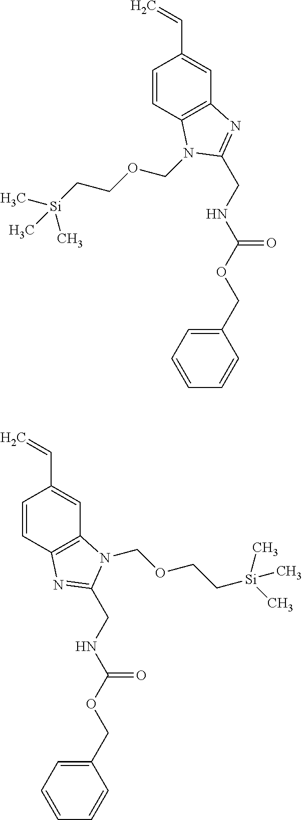 [SMILES: C=Cc1ccc2c(c1)nc(CNC(=O)OCc1ccccc1)n2COCC[Si](C)(C)C ; C=Cc1ccc2nc(CNC(=O)OCc3ccccc3)n(COCC[Si](C)(C)C)c2c1]

To a solution of benzyl [(5-bromo-1-{[2-(trimethylsilyl)ethoxy]methyl}-1H-benzimidazol-2-yl)methyl]carbamate-benzyl [(6-bromo-1-{[2-(trimethylsilyl)ethoxy]methyl}-1H-benzimidazol-2-yl)methyl]carbamate (1:1) (Intermediate 93, 5.80 g, 95% purity, 11.2 mmol) in a mixed solvent of 1,4-dioxane (100 mL) and water (20 mL) were added potassium trifluoro(vinyl)borate (3.01 g, 22.5 mmol, CAS-RN:[13682-77-4]), sodium carbonate (2.38 g, 22.5 mmol) and (1,1-bis(diphenylphosphino)ferrocene)-dichloropalladium(II) (411 mg, 0.562 mmol, CAS-RN:[72287-26-4]) at rt. The reaction mixture was heated to 90° C. and stirred at 90° C. for 12 h. The reaction mixture was cooled to rt and filtered through a pad of celite. Water was added to the filtrate. The solution was extracted with ethyl acetate. The organic layer was evaporated under reduced pressure to give a residue. The residue was purified by chromatography (200-300 mesh, petroleum ether: ethyl acetate=10:1 to 1:1) to give 4.10 g (91% purity, 76% yield) of the title compound as a yellow oil.

LC-MS (Method D): Rₜ=2.115 min; MS (ESIpos): m/z=438.2 [M+H]⁺.

### Intermediate 95

### benzyl {[5-(2-hydroxyethyl)-1-{[2-(trimethylsilyl)ethoxy]methyl}-1H-benzimidazol-2-yl]methyl}carbamate-benzyl {[6-(2-hydroxyethyl)-1-{[2-(trimethylsilyl)ethoxy]methyl}-1H-benzimidazol-2-yl]methyl}carbamate (1/1)

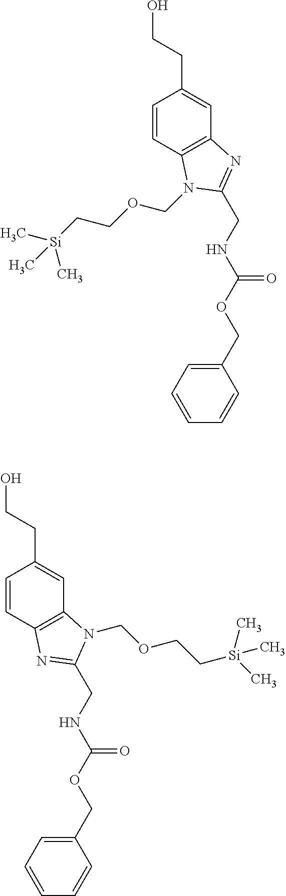 [SMILES: C[Si](C)(C)CCOCn1c(CNC(=O)OCc2ccccc2)nc2cc(CCO)ccc21 ; C[Si](C)(C)CCOCn1c(CNC(=O)OCc2ccccc2)nc2ccc(CCO)cc21]

To a solution of benzyl [(1-{[2-(trimethylsilyl)ethoxy]methyl}-5-vinyl-1H-benzimidazol-2-yl)methyl]carbamate-benzyl [(1-{[2-(trimethylsilyl)ethoxy]methyl}-6-vinyl-1H-benzimidazol-2-yl)methyl]carbamate (1:1) (Intermediate 94, 4.10 g, 91% purity, 8.53 mmol) in tetrahydrofuran (42 mL) was added 9-borabicyclo(3.3.1)nonane (51 mL, 0.50 M, 26 mmol, CAS-RN:[280-64-8]) at rt under nitrogen atmosphere. After stirring at rt for 12 h, ethanol (42 mL) was added to the reaction mixture. Then sodium hydroxide (10 mL, 1.0 M, 10 mmol) and hydrogen peroxide (10 mL, 330 mmol) were added to the reaction mixture. The reaction mixture was stirred at rt for 2 h. Saturated sodium thiosulfate was added to the reaction mixture. The solution was extracted with ethyl acetate and washed with glycerol/water (v/v=1/100). The organic layer was evaporated under reduced pressure to give a residue. The residue was purified by chromatography (1000 mesh, petroleum ether: ethyl acetate=10:1 then 0:1) to give 3.07 g (87% purity, 69% yield) of the title compound as a yellow oil.

LC-MS (Method D): Rₜ=0.751 min; MS (ESIpos): m/z=456.2 [M+H]⁺.

### Intermediate 96

### 2-[2-({[(benzyloxy)carbonyl]amino}methyl)-1-{[2-(trimethylsilyl)ethoxy]methyl}-1H-benzimidazol-5-yl]ethyl methanesulfonate-2-[2-({[(benzyloxy)carbonyl]amino}methyl)-1-{[2-(trimethylsilyl)ethoxy]methyl}-1H-benzimidazol-6-yl]ethyl methanesulfonate (1/1)

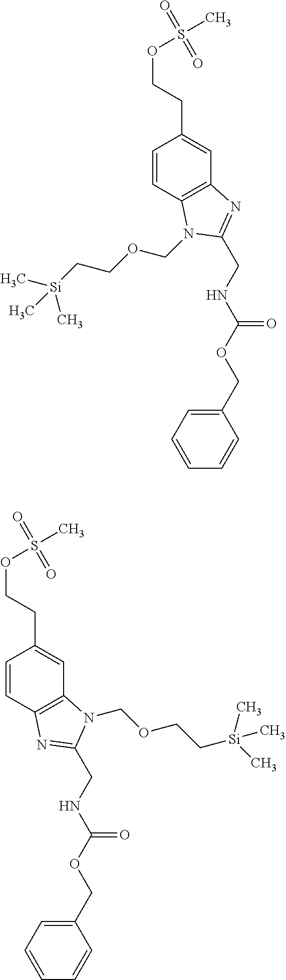 [SMILES: C[Si](C)(C)CCOCn1c(CNC(=O)OCc2ccccc2)nc2cc(CCOS(C)(=O)=O)ccc21 ; C[Si](C)(C)CCOCn1c(CNC(=O)OCc2ccccc2)nc2ccc(CCOS(C)(=O)=O)cc21]

To a solution of benzyl {[5-(2-hydroxyethyl)-1-{[2-(trimethylsilyl)ethoxy]methyl}-1H-benzimidazol-2-yl]methyl}carbamate-benzyl {[6-(2-hydroxyethyl)-1-{[2-(trimethylsilyl)ethoxy]methyl}-1H-benzimidazol-2-yl]methyl}carbamate (1:1) (Intermediate 95, 3.07 g, 85% purity, 5.73 mmol) and 4-methylsulfonyl chloride (0.530 mL, 6.9 mmol, CAS-RN:[124-63-0]) in dichloromethane (20 mL) was added triethylamine (2.4 mL, 17 mmol, CAS-RN:[121-44-8]) at 0° C. The reaction mixture was warmed to rt and stirred at rt for 1 h. The reaction mixture was evaporated under reduced pressure to give a residue. The residue was dissolved in ethyl acetate. The mixture was filtered, and the filtrate was evaporated under reduced pressure to give 4.35 g (70% purity) of the title compound as a yellow oil.

LC-MS (Method D): Rₜ=0.789 min; MS (ESIpos): m/z=534.2 [M+H]⁺.

### Intermediate 97

### benzyl {[5-(2-azidoethyl)-1-{[2-(trimethylsilyl)ethoxy]methyl}-1H-benzimidazol-2-yl]methyl}carbamate-benzyl {[6-(2-azidoethyl)-1-{[2-(trimethylsilyl)ethoxy]methyl}-1H-benzimidazol-2-yl]methyl}carbamate (1/1)

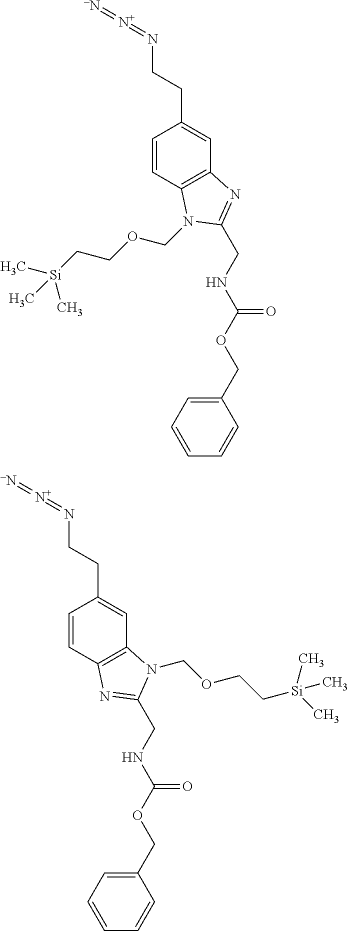 [SMILES: C[Si](C)(C)CCOCn1c(CNC(=O)OCc2ccccc2)nc2cc(CCN=[N+]=[N-])ccc21 ; C[Si](C)(C)CCOCn1c(CNC(=O)OCc2ccccc2)nc2ccc(CCN=[N+]=[N-])cc21]

To a solution of 2-[2-({[(benzyloxy)carbonyl]amino}methyl)-1-{[2-(trimethylsilyl)ethoxy]methyl}-1H-benzimidazol-5-yl]ethyl methanesulfonate-2-[2-({[(benzyloxy)carbonyl]amino}methyl)-1-{[2-(trimethylsilyl)ethoxy]methyl}-1H-benzimidazol-6-yl]ethyl methanesulfonate (1/1) (Intermediate 96, 4.35 g, 70% purity, 5.71 mmol) in N,N-dimethylformamide (20 mL) was added sodium azide (519 mg, 7.99 mmol, CAS-RN:[26628-22-8]) at rt. The reaction mixture was heated to 70° C. and stirred at 70° C. for 12 h. The reaction mixture was cooled to rt and diluted with water. The solution was extracted with ethyl acetate. The organic layer was evaporated to remove 3/4 volume of ethyl acetate and diluted with tetrahydrofuran to give 3.10 g (71% purity, 80% yield) of the title compound in tetrahydrofuran.

LC-MS (Method D): Rₜ=0.831 min; MS (ESIpos): m/z=481.2 [M+H]⁺.

### Intermediate 98

### benzyl {[5-(2-aminoethyl)-1-{[2-(trimethylsilyl)ethoxy]methyl}-1H-benzimidazol-2-yl]methyl}carbamate-benzyl {[6-(2-aminoethyl)-1-{[2-(trimethylsilyl)ethoxy]methyl}-1H-benzimidazol-2-yl]methyl}carbamate (1/1)

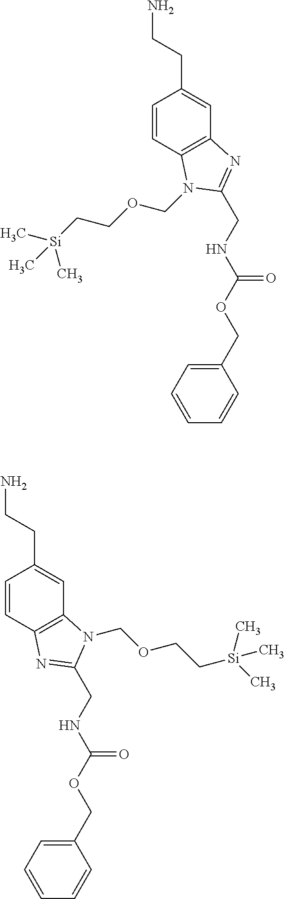 [SMILES: C[Si](C)(C)CCOCn1c(CNC(=O)OCc2ccccc2)nc2cc(CCN)ccc21 ; C[Si](C)(C)CCOCn1c(CNC(=O)OCc2ccccc2)nc2ccc(CCN)cc21]

To a solution of benzyl ((5-(2-azidoethyl)-1-((2-(trimethylsilyl)ethoxy)methyl)-1H-benzo[d]imidazol-2-yl)methyl)carbamate compound with benzyl ((6-(2-azidoethyl)-1-((2-(trimethylsilyl)ethoxy)methyl)-1H-benzo[d]imidazol-2-yl)methyl)carbamate (1:1) (Intermediate 97, 3.10 g, 71% purity, 4.58 mmol) in tetrahydrofuran (20 mL) were added water (0.500 mL) and triphenylphosphine (1.44 g, 5.50 mmol) at rt. The reaction mixture was heated to 50° C. and stirred at 50° C. for 12 h. The reaction mixture was cooled to rt and diluted with water. The solution was adjusted to pH˜4 by hydrochloric acid (1M in water) and washed with ethyl acetate. The organic layer was adjusted to pH 8˜ 9 by saturated sodium bicarbonate and extracted with ethyl acetate. The organic layer was evaporated under reduced pressure to give 2.25 g (92% purity, 99% yield) of the title compound as a yellow oil.

LC-MS (Method D): Rₜ=0.725 min; MS (ESIpos): m/z=455.3 [M+H]⁺.

### Intermediate 99

### benzyl [(5-{2-[(tert-butoxycarbonyl)amino]ethyl}-1-{[2-(trimethylsilyl)ethoxy]methyl}-1H-benzimidazol-2-yl)methyl]carbamate-benzyl [(6-{2-[(tert-butoxycarbonyl)amino]ethyl}-1-{[2-(trimethylsilyl)ethoxy]methyl}-1H-benzimidazol-2-yl)methyl]carbamate (1/1)

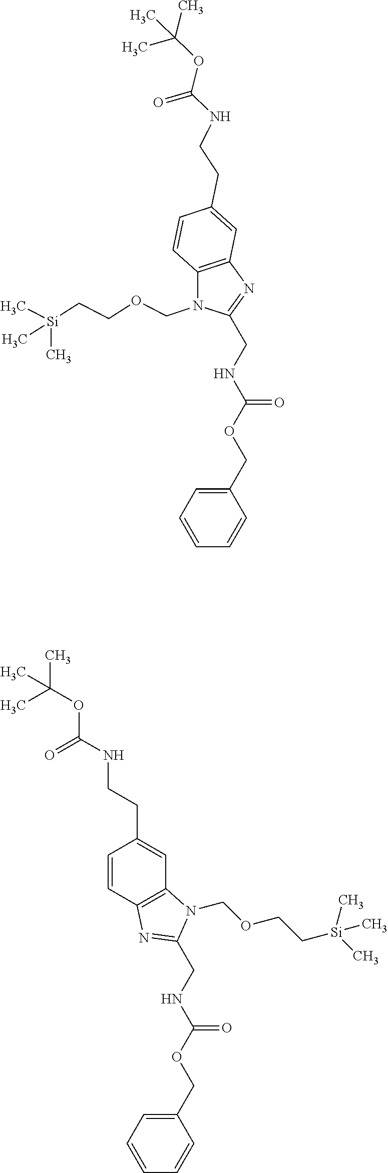 [SMILES: CC(C)(C)OC(=O)NCCc1ccc2c(c1)nc(CNC(=O)OCc1ccccc1)n2COCC[Si](C)(C)C ; CC(C)(C)OC(=O)NCCc1ccc2nc(CNC(=O)OCc3ccccc3)n(COCC[Si](C)(C)C)c2c1]

To a solution of benzyl ((5-(2-aminoethyl)-1-((2-(trimethylsilyl)ethoxy)methyl)-1H-benzo[d]imidazol-2-yl)methyl)carbamate compound with benzyl ((6-(2-aminoethyl)-1-((2-(trimethylsilyl)ethoxy)methyl)-1H-benzo[d]imidazol-2-yl)methyl)carbamate (1:1) (Intermediate 98, 2.25 g, 92% purity, 4.55 mmol) in tetrahydrofuran (20 mL) was added di-tert-butyl dicarbonate (1.3 mL, 5.5 mmol, CAS-RN:[24424-99-5]) at rt. After stirring at rt for 12 h, the reaction mixture was diluted with water. The solution was extracted with ethyl acetate. The organic layer was evaporated under reduced pressure to give a residue. The residue was purified by flash chromatography (200-300 mesh, petroleum ether:ethyl acetate=1:0 then 1:1) to give 1.40 g (81% purity, 45% yield) of the title compound as a colorless oil.

LC-MS (Method D): Rₜ=0.732 min; MS (ESIpos): m/z=555.3 [M+H]⁺.

### Intermediate 100

### tert-butyl {2-[2-(aminomethyl)-1-{[2-(trimethylsilyl)ethoxy]methyl}-1H-benzimidazol-5-yl]ethyl}carbamate-tert-butyl {2-[2-(aminomethyl)-1-{[2-(trimethylsilyl)ethoxy]methyl}-1H-benzimidazol-6-yl]ethyl}carbamate (1/1)

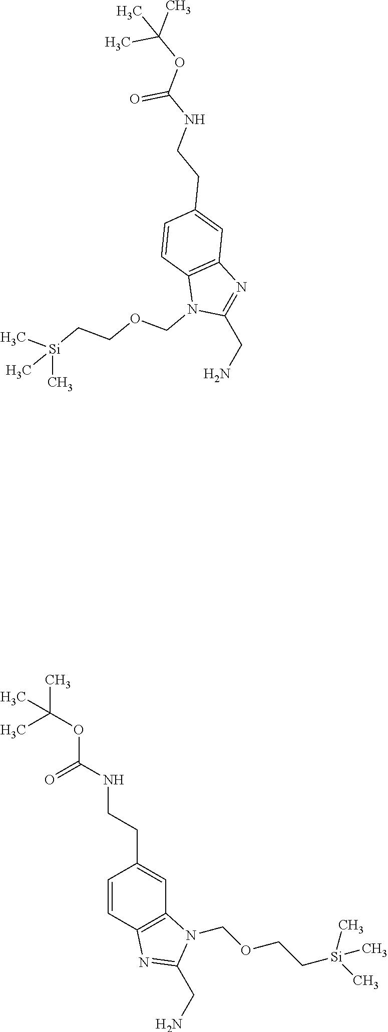 [SMILES: CC(C)(C)OC(=O)NCCc1ccc2c(c1)nc(CN)n2COCC[Si](C)(C)C ; CC(C)(C)OC(=O)NCCc1ccc2nc(CN)n(COCC[Si](C)(C)C)c2c1]

To a solution of benzyl [(5-{2-[(tert-butoxycarbonyl)amino]ethyl}-1-{[2-(trimethylsilyl)ethoxy]methyl}-1H-benzimidazol-2-yl)methyl]carbamate-benzyl [(6-{2-[(tert-butoxycarbonyl)amino]ethyl}-1-{[2-(trimethylsilyl)ethoxy]methyl}-1H-benzimidazol-2-yl)methyl]carbamate (1/1) (Intermediate 99, 4.30 g, 98% purity, 7.60 mmol) in methanol (50 mL) was added palladium on carbon (2.00 g, 10% purity, 1.88 mmol) at rt. The reaction mixture was stirred at rt for 2 h under hydrogen atmosphere. The reaction mixture was filtered through celite, the filtrate was evaporated under reduced pressure to give 2.64 g (94% purity, 78% yield) of the title compound as a colorless oil.

LC-MS (Method D): Rₜ=0.739 min; MS (ESIpos): m/z=421.2 [M+H]⁺.

¹H-NMR (400 MHz, DMSO-d₆): 5 [ppm]=7.58 (t, 1H), 7.48 (d, 1H), 7.16-7.09 (m, 1H), 6.96-6.91 (m, 1H), 5.70 (s, 2H), 4.05 (s, 2H), 3.65-3.55 (m, 2H), 3.28-3.21 (m, 2H), 2.90-2.84 (m, 2H), 1.50-1.39 (m, 9H), 0.94-0.89 (m, 2H), 0.07-0.07 (m, 9H).

### Intermediate 101

### tert-butyl {2-[2-({[8-bromo-2-(methylsulfanyl)pyrazolo[1,5-a][1,3,5]triazin-4-yl]amino}methyl)-1-{[2-(trimethylsilyl)ethoxy]methyl}-1H-benzimidazol-5-yl]ethyl}carbamate-tert-butyl {2-[2-({[8-bromo-2-(methylsulfanyl)pyrazolo[1,5-a][1,3,5]triazin-4-yl]amino}methyl)-1-{[2-(trimethylsilyl)ethoxy]methyl}-1H-benzimidazol-6-yl]ethyl}carbamate (1/1)

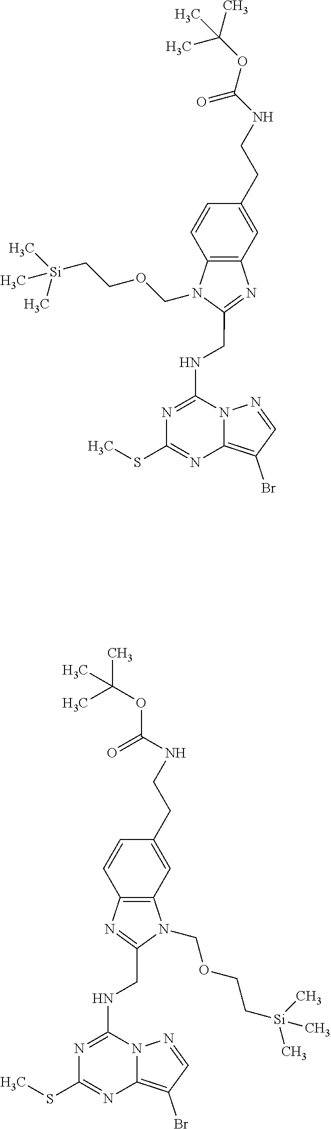

To a solution of tert-butyl {2-[2-(aminomethyl)-1-{[2-(trimethylsilyl)ethoxy]methyl}-1H-benzimidazol-5-yl]ethyl}carbamate-tert-butyl {2-[2-(aminomethyl)-1-{[2-(trimethylsilyl)ethoxy]methyl}-1H-benzimidazol-6-yl]ethyl}carbamate (1:1) (Intermediate 100, 790 mg, 89% purity, 1.67 mmol) in 1- butanol (8.9 mL) were added 8-bromo-4-chloro-2-(methylsulfanyl)pyrazolo[1,5-a][1,3,5]triazine (Intermediate 7, 788 mg, 89% purity, 2.51 mmol) and N,N-diisopropylethylamine (0.870 mL, 5.0 mmol) at rt. The reaction mixture was heated to 90° C. and stirred at 90° C. for 12 h. The reaction mixture was cooled to rt and evaporated under reduced pressure to give a residue. The residue was dissolved in ethyl acetate. The solution was washed with water. The organic layer was evaporated under reduced pressure to give a residue. The residue was purified by chromatography (1000 mesh, petroleum ether: ethyl acetate=20:1 then 1:1) to give 1.10 g (93% purity, 92% yield) of the title compound as a yellow oil.

LC-MS (Method D): Rₜ=0.939 min; MS (ESIpos): m/z=663.3 [M+H]⁺.

### Intermediate 102

### tert-butyl {2-[2-({[8-bromo-2-(methanesulfonyl)pyrazolo[1,5-a][1,3,5]triazin-4-yl]amino}methyl)-1-{[2-(trimethylsilyl)ethoxy]methyl}-1H-benzimidazol-5-yl]ethyl}carbamate-tert-butyl {2-[2-({[8-bromo-2-(methanesulfonyl)pyrazolo[1,5-a][1,3,5]triazin-4-yl]amino}methyl)-1-{[2-(trimethylsilyl)ethoxy]methyl}-1H-benzimidazol-6-yl]ethyl}carbamate (1/1)

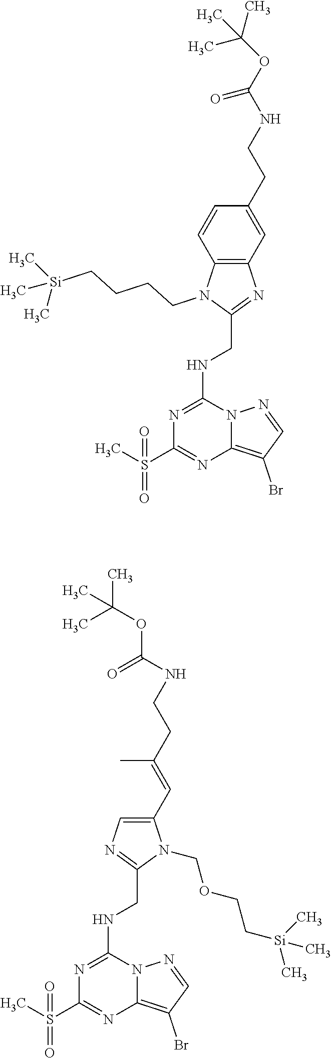

To a solution of tert-butyl {2-[2-({[8-bromo-2-(methylsulfanyl)pyrazolo[1,5-a][1,3,5]triazin-4-yl]amino}methyl)-1-{[2-(trimethylsilyl)ethoxy]methyl}-1H-benzimidazol-5-yl]ethyl}carbamate-tert-butyl {2-[2-({[8-bromo-2-(methylsulfanyl)pyrazolo[1,5-a][1,3,5]triazin-4-yl]amino}methyl)-1-{[2-(trimethylsilyl)ethoxy]methyl}-1H-benzimidazol-6-yl]ethyl}carbamate (1:1) (Intermediate 101, 1.10 g, 93% purity, 1.54 mmol) in dichloromethane (20 mL) was added meta-chloroperoxybenzoic acid (798 mg, 4.62 mmol, CAS-RN:[937-14-4]) at rt. The reaction mixture was stirred at rt for 2 h. Water was added to the reaction mixture. The solution was extracted with ethyl acetate. The organic layer was evaporated under reduced pressure to give 1.80 g (55% purity, 92% yield) of the title compound as a yellow oil.

LC-MS (Method D): Rₜ=0.860 min; MS (ESIpos): m/z=695.1 [M+H]⁺.

### Intermediate 103

### tert-butyl {2-[2-({[8-bromo-2-(morpholin-4-yl)pyrazolo[1,5-a][1,3,5]triazin-4-yl]amino}methyl)-1-{[2-(trimethylsilyl)ethoxy]methyl}-1H-benzimidazol-5-yl]ethyl}carbamate-tert-butyl {2-[2-({[8-bromo-2-(morpholin-4-yl)pyrazolo[1,5-a][1,3,5]triazin-4-yl]amino}methyl)-1-{[2-(trimethylsilyl)ethoxy]methyl}-1H-benzimidazol-6-yl]ethyl}carbamate (1/1)

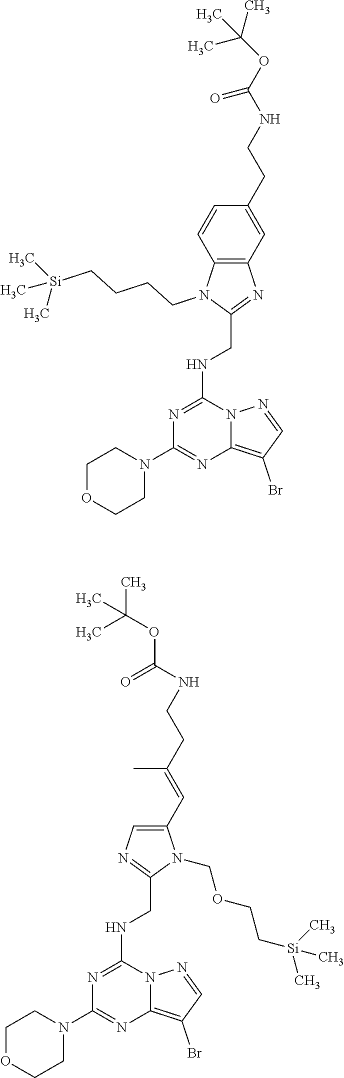

To a solution of tert-butyl {2-[2-({[8-bromo-2-(methylsulfonyl)pyrazolo[1,5-a][1,3,5]triazin-4-yl]amino}methyl)-1-{[2-(trimethylsilyl)ethoxy]methyl}-1H-benzimidazol-5-yl]ethyl}carbamate-tert-butyl {2-[2-({[8-bromo-2-(methylsulfonyl)pyrazolo[1,5-a][1,3,5]triazin-4-yl]amino}methyl)-1-{[2-(trimethylsilyl)ethoxy]methyl}-1H-benzimidazol-6-yl]ethyl}carbamate (1:1) (Intermediate 102, 1.80 g, 55% purity, 1.42 mmol) in acetonitrile (20 mL) were added morpholine (372 mg, 4.27 mmol, CAS-RN:[110-91-8]) and N,N-diisopropylethylamine (0.740 mL, 4.3 mmol) at rt. The reaction mixture was heated to 80° C. and stirred at 80° C. for 12 h. The reaction mixture was evaporated under reduced pressure to give a residue. The residue was purified by chromatography (1000 mesh, petroleum ether: ethyl acetate=10:1 then 1:1) to give 770 mg (78% purity, 60% yield) of the title compound as a yellow oil.

LC-MS (Method D): Rₜ=0.864 min; MS (ESIpos): m/z=702.2 [M+H]⁺.

### Intermediate 104

### 8-bromo-N-[(5-methoxy-1H-benzimidazol-2-yl)methyl]-2-(methylsulfanyl)pyrazolo[1,5-a][1,3,5]triazin-4-amine hydrogen chloride (1/1)

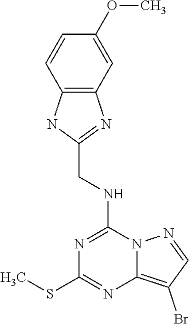 [SMILES: COc1ccc2[nH]c(CNc3nc(SC)nc4c(Br)cnn34)nc2c1]

1-(5-methoxy-1H-benzimidazol-2-yl)methanamine hydrogen chloride (1/2) (1.00 g, 4.00 mmol) was dissolved in dichloromethane (20 mL), N,N-diisopropylethylamine (1.4 mL, 8.0 mmol; CAS-RN:[7087-68-5]) and 8-bromo-4-chloro-2-(methylsulfanyl)pyrazolo[1,5-a][1,3,5]triazine (Intermediate 7, 1.12 g, 4.00 mmol) were added and the solution was stirred for 1 h at rt. The reaction mixture was concentrated and the residue was washed with water and ethanol to give 1.48 g (66% yield) of the title compound, which was used without further purification.

LC-MS (Method 2): Rₜ=1.10 min; MS (ESIpos): m/z=420 [M+H]⁺

¹H-NMR (400 MHz, DMSO-d6) δ[ppm]: 1.248 (0.77), 1.255 (0.48), 1.264 (0.84), 1.269 (0.80), 1.285 (0.70), 2.402 (2.64), 2.405 (2.87), 2.420 (14.11), 2.521 (2.01), 2.525 (1.34), 2.572 (1.96), 2.632 (2.53), 2.644 (2.36), 3.678 (2.83), 3.784 (2.66), 3.800 (16.00), 5.019 (2.15), 5.034 (2.15), 6.932 (1.06), 6.938 (1.02), 6.946 (0.46), 6.954 (1.03), 6.960 (1.04), 7.082 (1.99), 7.088 (1.87), 7.096 (0.60), 7.102 (0.48), 7.274 (0.43), 7.281 (0.41), 7.376 (0.48), 7.398 (0.42), 7.494 (1.88), 7.516 (1.72), 7.591 (0.49), 7.613 (0.45), 8.120 (0.79), 8.212 (1.17), 8.239 (1.08), 8.313 (7.03), 8.439 (1.34), 8.484 (1.32), 9.619 (0.56), 9.634 (1.23), 9.648 (0.57).

### Intermediate 105

### 8-bromo-2-(methanesulfinyl)-N-[(5-methoxy-1H-benzimidazol-2-yl)methyl]pyrazolo[1,5-a][1,3,5]triazin-4-amine

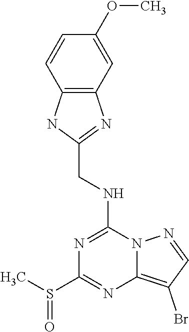 [SMILES: COc1ccc2[nH]c(CNc3nc(S(C)=O)nc4c(Br)cnn34)nc2c1]

8-bromo-N-[(4-methoxy-1H-benzimidazol-2-yl)methyl]-2-(methylsulfanyl)pyrazolo[1,5-a][1,3,5]triazin-4-amine hydrogen chloride (1/1) (Intermediate 104, 200 mg, 359 μmol) was dissolved in dichloromethane (10 mL), cooled to 0° C., mCPBA (241 mg, 77% purity, 1.08 mmol; CAS-RN:[937-14-4]) was added and the mixture was stirred for 0.5 h at 0° C. The mixture was treated with aqueous saturated sodium bicarbonate solution and extracted with dichloromethane. The combined organic layers were dried, filtered and concentrated. The residue was purified by flash chromatography to give 133 mg (70% yield) of the title compound.

¹H-NMR (400 MHz, DMSO-d6) δ[ppm]: 0.817 (0.41), 0.824 (0.43), 0.886 (0.83), 0.904 (1.86), 0.907 (0.62), 0.923 (0.96), 0.994 (0.42), 1.037 (2.26), 1.055 (5.54), 1.073 (2.55), 1.234 (1.11), 2.068 (2.37), 2.325 (0.67), 2.329 (0.93), 2.334 (0.65), 2.422 (0.52), 2.441 (0.53), 2.521 (3.47), 2.525 (2.23), 2.648 (0.50), 2.654 (0.51), 2.667 (1.31), 2.671 (1.19), 2.676 (0.72), 2.728 (0.42), 2.738 (0.43), 2.755 (1.76), 2.759 (1.91), 2.769 (6.11), 3.273 (0.79), 3.424 (0.81), 3.437 (0.87), 3.442 (0.87), 3.455 (0.88), 3.579 (1.02), 3.672 (0.56), 3.742 (1.47), 3.749 (16.00), 3.766 (0.47), 3.778 (0.60), 3.835 (0.60), 3.842 (0.58), 4.345 (0.52), 4.358 (0.99), 4.370 (0.47), 4.942 (1.37), 4.953 (1.37), 6.762 (0.60), 6.782 (0.63), 7.529 (0.45), 7.550 (0.94), 7.570 (0.57), 7.888 (0.54), 7.890 (0.42), 7.895 (0.56), 7.899 (0.70), 7.904 (0.91), 7.907 (0.52), 8.501 (7.21), 8.505 (0.73), 8.512 (0.56), 8.579 (0.56), 10.045 (0.47), 10.059 (0.91), 10.073 (0.47).

### Intermediate 106

### N-[8-bromo-2-(methylsulfanyl)pyrazolo[1,5-a][1,3,5]triazin-4-yl]glycine

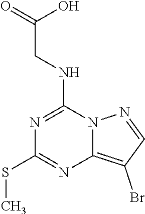 [SMILES: CSc1nc(NCC(=O)O)n2ncc(Br)c2n1]

To methyl N-[8-bromo-2-(methylsulfanyl)pyrazolo[1,5-a][1,3,5]triazin-4-yl]glycinate (Intermediate 35, 5.49 g, 85% purity, 14.0 mmol) were added THF (40 mL), MeOH (10 mL) and lithium hydroxide solution (28 mL, 1.0 M, 28 mmol; CAS-RN:[1310-65-2]). The reaction mixture was stirred for 24 h at rt. The solution was treated with water and aqueous citric acid solution (10%) was added to adjust the pH to 2-3. The suspension was concentrated under reduced pressure and the residue was cooled by an ice-bath. The precipitate was filtered off and washed with a small amount of water. The filter cake was dried at 50° C. under reduced pressure to give 4.97 (crude) of the title compound as a white solid.

LC-MS (Method 1): Rₜ=0.93 min; MS (ESIpos): m/z=318 [M+H]⁺

¹H-NMR (400 MHz, DMSO-d6) δ[ppm]: 2.518 (1.23), 2.523 (0.85), 4.119 (6.03), 4.134 (5.92), 8.268 (16.00), 9.233 (1.13), 9.248 (2.34), 9.264 (1.07).

### Intermediate 107

### 2-{[8-bromo-2-(methylsulfanyl)pyrazolo[1,5-a][1,3,5]triazin-4-yl]amino}acetohydrazide

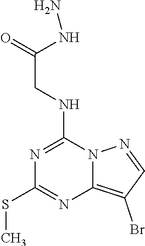 [SMILES: CSc1nc(NCC(=O)NN)n2ncc(Br)c2n1]

N-[8-bromo-2-(methylsulfanyl)pyrazolo[1,5-a][1,3,5]triazin-4-yl]glycine (Intermediate 106, 4.96 g, 15.6 mmol) was dissolved in THF (100 mL), di(1H-imidazol-1-yl)methanone (5.06 g, 31.2 mmol; CAS-RN:[530-62-1]) was added. The reaction mixture was refluxed for 6 h. The solution was cooled to rt and hydrazine in THF (78 mL, 1.0 M, 78 mmol) was added dropwise while cooling with ice-bath. The reaction mixture was stirred for 22 h at rt. The suspension was treated with water and a small amount of ethanol. The precipitate was filtered off and washed with ethanol. The filtrate was concentrated under reduced pressure and extracted with ethylacetate. The combined organic layer was washed with brine, filtered over a water repellent filter and dried under reduced pressure. The residue was stirred with methyltertbutylether and the precipitate was filtered off and dried to give 1.98 (34% yield) of the title compound as a yellow solid.

LC-MS (Method 1): Rₜ=0.80 min; MS (ESIpos): m/z=332 [M+H]⁺

¹H-NMR (400 MHz, DMSO-d6) δ[ppm]: −0.008 (0.64), 0.008 (0.44), 1.175 (0.64), 1.879 (4.29), 1.910 (0.62), 1.938 (1.30), 1.953 (3.39), 1.990 (1.23), 2.483 (7.93), 2.521 (3.18), 2.526 (2.06), 4.020 (0.65), 4.035 (4.84), 4.049 (4.69), 4.372 (0.62), 4.386 (0.62), 4.441 (0.77), 4.456 (0.75), 4.518 (0.56), 7.058 (2.29), 7.731 (0.70), 8.235 (16.00), 8.243 (1.69), 8.251 (1.11), 8.258 (0.58), 8.268 (1.61), 8.687 (0.43), 9.071 (0.96), 9.086 (1.70), 9.100 (0.90), 9.208 (3.25), 10.470 (0.62).

### Intermediate 108

### 8-bromo-N-{[5-(3-fluorophenyl)-4H-1,2,4-triazol-3-yl]methyl}-2-(methylsulfanyl)pyrazolo[1,5-a][1,3,5]triazin-4-amine

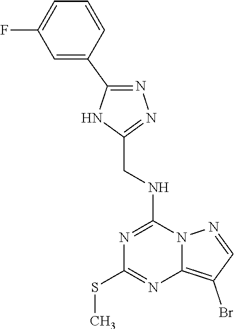 [SMILES: CSc1nc(NCc2nnc(-c3cccc(F)c3)[nH]2)n2ncc(Br)c2n1]

2-{[8-bromo-2-(methylsulfanyl)pyrazolo[1,5-a][1,3,5]triazin-4-yl]amino}acetohydrazide (Intermediate 107, 1.88 g, 5.66 mmol) and 3-fluorobenzene-1-carboximidamide hydrogen chloride (1/1) (1.19 g, 6.79 mmol, CAS-RN:[75207-72-6]) were dissolved in DMF (30 mL). Sodiumethylate (770 mg, 11.3 mmol; CAS-RN:[141-52-6]) was added and the reaction mixture was stirred for 1 h at 180° C. in the microwave. The reaction mixture was poured into water and was extracted with ethylacetate. The combined organic layer was washed with brine, filtered over a water repellent filter and concentrated under reduced pressure. The residue was purified by two flash chromatographies (silica gel, first: dichloromethane/ethanol gradient, second: hexane/ethylacetate gradient) to give 725 mg (26% yield) of the title compound as a light brown solid.

LC-MS (Method 1): Rₜ=1.20 min; MS (ESIpos): m/z=435 [M+H]⁺

¹H-NMR (400 MHz, DMSO-d6) δ[ppm]: 1.157 (2.41), 1.175 (4.81), 1.192 (2.41), 1.234 (0.45), 1.910 (0.40), 1.990 (8.85), 1.997 (1.37), 2.002 (1.07), 2.454 (16.00), 4.002 (0.63), 4.019 (1.94), 4.038 (1.96), 4.056 (0.67), 4.870 (3.24), 7.254 (1.44), 7.519 (1.63), 7.688 (2.10), 7.710 (2.20), 7.811 (4.36), 7.831 (3.86), 8.281 (11.96), 9.603 (1.45), 14.009 (1.29).

### Intermediate 109

### 8-bromo-N-{[5-(3-fluorophenyl)-4H-1,2,4-triazol-3-yl]methyl}-2-(methanesulfonyl)pyrazolo[1,5-a][1,3,5]triazin-4-amine

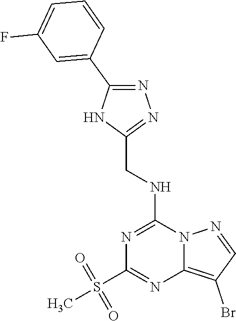 [SMILES: CS(=O)(=O)c1nc(NCc2nnc(-c3cccc(F)c3)[nH]2)n2ncc(Br)c2n1]

8-bromo-N-{[5-(3-fluorophenyl)-4H-1,2,4-triazol-3-yl]methyl}-2-(methylsulfanyl)pyrazolo[1,5-a][1,3,5]triazin-4-amine (725 mg, 1.50 mmol) was suspended in dichloromethane (10 mL). The suspension was cooled by ice-bath to 2° C. and meta-chloroperoxybenzoic acid (1.01 g, 77% purity, 4.50 mmol; CAS-RN:[937-14-4]) was added. The reaction mixture was stirred at rt for 5 h. Meta-chloroperoxybenzoic acid (0.34 g, 77% purity, 1.50 mmol; CAS-RN:[937-14-4]) was added. The reaction mixture was stirred at rt for 16 h. Aqueous sodiumthiosulfate solution was added to the reaction mixture and was stirred for 1 h at rt. The precipitate was filtered off, washed with water and dichloromethane and dried under vacuum at 50° C. to give 1.11 g (58% purity, 92% yield) of the title compound as a white solid in a mixture with the corresponding sulfoxide.

LC-MS (Method 1): Rₜ=0.98 min; MS (ESIpos): m/z=467 [M+H]⁺

¹H-NMR (400 MHz, DMSO-d6) δ[ppm]: 1.909 (4.51), 2.336 (0.66), 2.522 (3.68), 2.526 (2.32), 2.678 (0.72), 2.806 (2.63), 2.934 (0.59), 3.120 (0.72), 3.303 (16.00), 4.850 (0.76), 4.916 (0.78), 4.963 (3.79), 7.248 (0.63), 7.268 (0.93), 7.274 (0.91), 7.289 (0.48), 7.295 (0.50), 7.413 (2.60), 7.432 (6.13), 7.451 (4.19), 7.497 (0.66), 7.520 (3.23), 7.523 (3.44), 7.526 (3.39), 7.528 (3.26), 7.539 (2.15), 7.543 (2.19), 7.545 (2.23), 7.548 (1.92), 7.707 (0.95), 7.726 (0.74), 7.729 (0.80), 7.822 (1.74), 7.828 (3.59), 7.831 (5.70), 7.835 (3.87), 7.842 (1.85), 7.847 (2.78), 7.850 (6.07), 7.854 (8.17), 7.859 (6.03), 7.862 (3.57), 8.243 (1.70), 8.475 (1.03), 8.510 (2.47).

### Intermediate 110

### 8-bromo-N-[(4-methyl-1H-benzimidazol-2-yl)methyl]-2-(methylsulfanyl)pyrazolo[1,5-a][1,3,5]triazin-4-amine

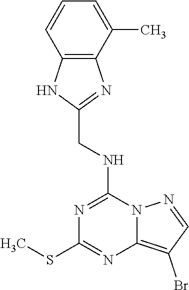 [SMILES: CSc1nc(NCc2nc3c(C)cccc3[nH]2)n2ncc(Br)c2n1]

To dichloromethane (20 mL) was added 1-(4-methyl-1H-benzimidazol-2-yl)methanamine hydrogen chloride (1/2) (1.00 g, 4.27 mmol, CAS-RN:[1269087-76-4]), N,N-diisopropylethylamine (2.2 mL, 13 mmol; CAS-RN:[7087-68-5]) and 8-bromo-4-chloro-2-(methylsulfanyl)pyrazolo[1,5-a][1,3,5]triazine (Intermediate 7, 1.19 g, 4.27 mmol). The reaction mixture was stirred for 5 h at rt. The mixture was concentrated under reduced pressure and treated with water. The precipitate was filtered off, washed with water and ethanol and dried at 60° C. under reduced pressure to give 1.84 g (96% yield) of the title compound.

LC-MS (Method 2): Rₜ=1.18 min; MS (ESIpos): m/z=404 [M+H]⁺

¹H-NMR (400 MHz, DMSO-d6) δ[ppm]: 1.234 (1.55), 1.246 (4.78), 1.252 (3.96), 1.263 (6.23), 1.283 (4.15), 2.446 (16.00), 2.469 (1.41), 2.484 (8.49), 2.521 (1.80), 2.525 (1.17), 3.120 (0.55), 3.131 (0.51), 3.139 (0.56), 3.149 (0.48), 3.607 (0.47), 3.617 (0.43), 4.933 (2.45), 4.948 (2.44), 6.938 (1.17), 6.956 (1.73), 7.017 (1.62), 7.037 (2.16), 7.055 (1.24), 8.288 (7.32), 9.552 (0.54).

### Intermediate 111

### 8-bromo-N-[(4-methyl-1H-benzimidazol-2-yl)methyl]-2-methylsulfinyl-pyrazolo[1,5-a][1,3,5]triazin-4-amine

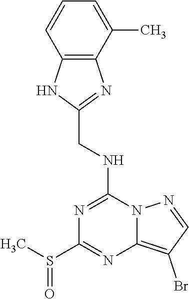 [SMILES: Cc1cccc2[nH]c(CNc3nc(S(C)=O)nc4c(Br)cnn34)nc12]

8-bromo-N-[(4-methyl-1H-benzimidazol-2-yl)methyl]-2-(methylsulfanyl)pyrazolo[1,5-a][1,3,5]triazin-4-amine (Intermediate 110, 1.73 g, 4.27 mmol) was suspended in dichloromethane (50 mL). To the ice-bath cooled suspension was added in portions 3-chlorobenzene-1-carboperoxoic acid (2.87 g, 77% purity, 12.8 mmol; CAS-RN:[937-14-4]). The reaction mixture was stirred over night at rt. An aqueous saturated sodium bicarbonate solution was added and the organic layer was separated. The aqueous layer was extracted with dichloromethane. The combined organic layers were dried over sodiumsulfate, filtrated and concentrated. The residue was purified by flash chromatography (silica; hexane/ethylacetate gradient) to give 680 mg (38% yield) of the title compound.

LC-MS (Method 2): Rₜ=0.80 min; MS (ESIpos): m/z=420 [M+H]⁺

### Intermediate 112

### 8-bromo-N-[(5-methyl-1H-benzimidazol-2-yl)methyl]-2-(methylsulfanyl)pyrazolo[1,5-a][1,3,5]triazin-4-amine

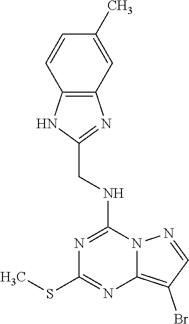 [SMILES: CSc1nc(NCc2nc3cc(C)ccc3[nH]2)n2ncc(Br)c2n1]

To dichloromethane (20 mL) was added 1-(5-methyl-1H-benzimidazol-2-yl)methanamine hydrogen chloride (1/2) (1.00 g, 4.27 mmol, CAS-RN:[89219-02-3]), N,N-diisopropylethylamine (2.2 mL, 13 mmol; CAS-RN:[7087-68-5]) and 8-bromo-4-chloro-2-(methylsulfanyl)pyrazolo[1,5-a][1,3,5]triazine (Intermediate 7, 1.19 g, 4.27 mmol). The reaction mixture was stirred over the weekend at rt. The mixture was concentrated under reduced pressure and treated with water. The precipitate was filtered off, washed with water and ethanol and dried at 60° C. under reduced pressure to give 1.85 g (92% yield) of the title compound.

LC-MS (Method 2): Rₜ=1.17 min; MS (ESIpos): m/z=404 [M+H]⁺

¹H-NMR (400 MHz, DMSO-d6) δ[ppm]: 1.234 (1.78), 1.246 (5.80), 1.252 (4.23), 1.263 (7.29), 1.270 (2.64), 1.282 (5.24), 2.387 (10.10), 2.417 (0.86), 2.433 (16.00), 2.466 (0.53), 2.521 (2.10), 2.526 (1.46), 2.535 (2.73), 3.120 (0.74), 3.130 (0.72), 3.139 (0.74), 3.149 (0.68), 3.591 (0.44), 3.600 (0.44), 3.607 (0.58), 3.617 (0.56), 3.624 (0.44), 3.633 (0.40), 4.904 (2.41), 4.919 (2.37), 6.963 (1.15), 6.966 (1.15), 6.984 (1.29), 6.987 (1.25), 7.267 (1.49), 7.353 (1.21), 7.373 (1.09), 8.032 (0.45), 8.286 (8.03), 9.537 (0.49), 9.552 (1.00), 9.567 (0.47).

### Intermediate 113

### 8-bromo-N-[(5-methyl-1H-benzimidazol-2-yl)methyl]-2-methylsulfinyl-pyrazolo[1,5-a][1,3,5]triazin-4-amine

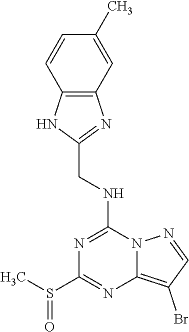 [SMILES: Cc1ccc2[nH]c(CNc3nc(S(C)=O)nc4c(Br)cnn34)nc2c1]

8-bromo-N-[(5-methyl-1H-benzimidazol-2-yl)methyl]-2-(methylsulfanyl)pyrazolo[1,5-a][1,3,5]triazin-4-amine (Intermediate 112, 1.85 g, 4.58 mmol) was suspended in dichloromethane (100 mL). To the ice-bath cooled suspension was added in portions 3-chlorobenzene-1-carboperoxoic acid (3.08 g, 77% purity, 13.7 mmol; CAS-RN:[937-14-4]). The reaction mixture was stirred over night at rt. Aqueous saturated sodium bicarbonate solution was added and the organic layer was separated. The aqueous layer was extracted with dichloromethane. The combined organic layers were dried over sodiumsulfate, filtrated and concentrated to give 2.35 g (85% yield) of the title compound.

LC-MS (Method 2): Rₜ=0.82 min; MS (ESIpos): m/z=419 [M+H]⁺

### Intermediate 114

### 8-cyclopropyl-N-[(4,5-difluoro-1H-benzimidazol-2-yl)methyl]-2-(methylsulfanyl)pyrazolo[1,5-a][1,3,5]triazin-4-amine

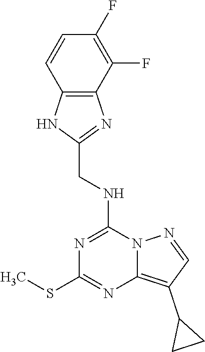 [SMILES: CSc1nc(NCc2nc3c(F)c(F)ccc3[nH]2)n2ncc(C3CC3)c2n1]

To dichloromethane (20 mL) was added 1-(4,5-difluoro-1H-benzimidazol-2-yl)methanamine hydrogen chloride (1/2) (947 mg, 3.70 mmol, CAS-RN:[1201769-17-6]), N,N-diisopropylethylamine (1.9 mL, 11 mmol; CAS-RN:[7087-68-5]) and 4-chloro-8-cyclopropyl-2-(methylsulfanyl)pyrazolo[1,5-a][1,3,5]triazine (Intermediate 55, 890 mg, 3.70 mmol). The reaction mixture was stirred for 5 h at rt. The mixture was concentrated under reduced pressure and water was added to the residue. The precipitate was filtered off, washed with water and ethanol and dried at 60° C. under reduced pressure to give 1.45 g (91% yield) of the title compound.

LC-MS (Method 2): Rₜ=1.22 min; MS (ESIpos): m/z=388 [M+H]⁺

¹H-NMR (400 MHz, DMSO-d6) δ[ppm]: 0.812 (0.92), 0.825 (2.51), 0.830 (2.72), 0.837 (3.76), 0.843 (2.72), 0.851 (1.59), 0.854 (1.06), 0.865 (2.28), 0.872 (2.76), 0.877 (2.02), 0.882 (1.16), 0.886 (1.71), 0.893 (3.08), 0.898 (1.99), 0.905 (0.53), 0.911 (0.97), 1.157 (1.85), 1.175 (3.76), 1.193 (1.86), 1.845 (0.50), 1.857 (0.92), 1.865 (0.86), 1.875 (0.87), 1.878 (1.37), 1.887 (0.54), 1.891 (0.82), 1.899 (0.77), 1.990 (6.39), 2.374 (1.41), 2.401 (16.00), 2.466 (0.47), 2.476 (0.79), 2.521 (3.06), 2.526 (1.84), 2.532 (0.44), 2.584 (1.34), 3.348 (1.29), 4.002 (0.48), 4.020 (1.45), 4.038 (1.46), 4.056 (0.47), 4.897 (3.05), 4.912 (3.06), 7.189 (0.80), 7.205 (1.12), 7.210 (2.07), 7.215 (1.28), 7.226 (2.28), 7.232 (0.67), 7.854 (0.48), 7.935 (0.78), 7.944 (4.96), 8.104 (0.59), 9.296 (0.58), 9.311 (1.21), 9.326 (0.57), 12.699 (1.21).

### Intermediate 115

### 8-cyclopropyl-N-[(4,5-difluoro-1H-benzimidazol-2-yl)methyl]-2-(methanesulfonyl)pyrazolo[1,5-a][1,3,5]triazin-4-amine

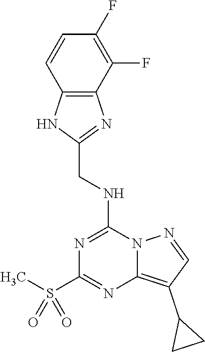 [SMILES: CS(=O)(=O)c1nc(NCc2nc3c(F)c(F)ccc3[nH]2)n2ncc(C3CC3)c2n1]

8-cyclopropyl-N-[(4,5-difluoro-1H-benzimidazol-2-yl)methyl]-2-(methylsulfanyl)pyrazolo[1,5-a][1,3,5]triazin-4-amine (Intermediate 114, 1.45 g, 3.74 mmol) was suspended in dichloromethane (80 mL). To the ice-bath cooled suspension was added in portions 3-chlorobenzene-1-carboperoxoic acid (2.52 g, 77% purity, 11.2 mmol; CAS-RN:[937-14-4]). The reaction mixture was stirred over night at rt. Saturated sodium bicarbonate solution was added and the organic layer was separated. The an organic layer was extracted with dichloromethane. The combined organic layer was dried over sodiumsulfate, filtrated and concentrated to give 1.42 g (81% yield) of the title compound.

LC-MS (Method 2): Rₜ=0.94 min; MS (ESIpos): m/z=420 [M+H]⁺

¹H-NMR (400 MHz, DMSO-d6) δ[ppm]: −0.008 (1.84), 0.008 (1.69), 0.853 (1.11), 0.864 (2.64), 0.870 (2.86), 0.877 (2.79), 0.883 (2.86), 0.892 (1.41), 0.958 (1.50), 0.967 (2.73), 0.973 (2.21), 0.979 (1.72), 0.984 (1.35), 0.988 (2.86), 0.994 (2.09), 1.005 (0.95), 1.175 (0.64), 1.966 (0.49), 1.979 (0.92), 1.987 (1.01), 1.990 (1.41), 2.000 (1.72), 2.009 (0.55), 2.013 (0.83), 2.021 (0.80), 2.339 (0.46), 2.521 (4.98), 2.525 (3.29), 2.676 (1.07), 2.681 (0.46), 3.233 (16.00), 3.249 (0.74), 3.271 (0.58), 3.319 (2.03), 5.025 (2.49), 5.038 (2.49), 5.761 (3.93), 7.200 (0.86), 7.218 (2.24), 7.226 (1.29), 7.231 (1.47), 7.236 (2.03), 8.214 (5.19), 10.012 (0.80), 12.726 (1.04).

### Intermediate 116

### 8-bromo-N-[(5-methoxy-1H-benzimidazol-2-yl)methyl]-2-(methylsulfanyl)pyrazolo[1,5-a][1,3,5]triazin-4-amine

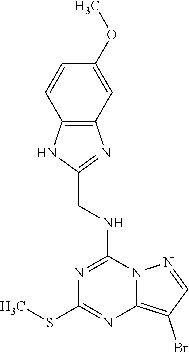 [SMILES: COc1ccc2[nH]c(CNc3nc(SC)nc4c(Br)cnn34)nc2c1]

To dichloromethane (10 mL) was added 1-(5-methoxy-1H-benzimidazol-2-yl)methanamine hydrogen chloride (1/2) (500 mg, 2.00 mmol, CAS-RN:[1255717-63-5]), triethylamine (840 μL, 6.0 mmol; CAS-RN:[121-44-8]) and 8-bromo-4-chloro-2-(methylsulfanyl)pyrazolo[1,5-a][1,3,5]triazine (Intermediate 7, 559 mg, 2.00 mmol). The reaction mixture was stirred over night at rt. The mixture was concentrated under reduced pressure and water was added to the residue. The precipitate was filtered off, washed with water and ethanol and dried at 60° C. under reduced pressure to give 827 mg (85% yield) of the title compound.

LC-MS (Method 2): Rₜ=1.10 min; MS (ESIpos): m/z=420 [M+H]⁺

¹H-NMR (400 MHz, DMSO-d6) δ[ppm]: 1.246 (1.03), 1.252 (0.67), 1.264 (1.26), 1.283 (0.95), 2.406 (0.42), 2.430 (13.39), 2.452 (0.46), 2.521 (2.18), 2.526 (1.32), 2.571 (3.77), 3.780 (16.00), 3.800 (0.43), 4.961 (2.32), 4.975 (2.33), 6.856 (1.04), 6.862 (1.11), 6.878 (1.11), 6.884 (1.17), 7.037 (1.88), 7.043 (1.82), 7.433 (1.96), 7.455 (1.79), 8.116 (1.39), 8.302 (6.58), 9.579 (0.57), 9.593 (1.23), 9.608 (0.59).

### Intermediate 117

### 8-bromo-N-[(5-methoxy-1H-benzimidazol-2-yl)methyl]-2-methylsulfinyl-pyrazolo[1,5-a][1,3,5]triazin-4-amine and 8-bromo-N-[(5-methoxy-1H-benzimidazol-2-yl)methyl]-2-methylsulfonyl-pyrazolo[1,5-a][1,3,5]triazin-4-amine

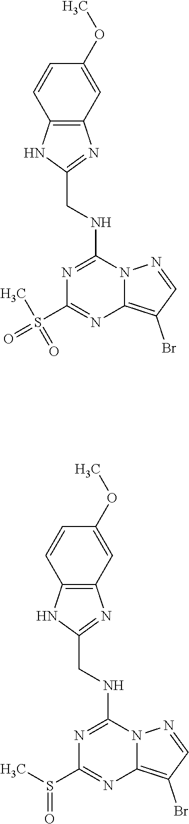 [SMILES: COc1ccc2[nH]c(CNc3nc(S(C)(=O)=O)nc4c(Br)cnn34)nc2c1 ; COc1ccc2[nH]c(CNc3nc(S(C)=O)nc4c(Br)cnn34)nc2c1]

8-bromo-N-[(5-methoxy-1H-benzimidazol-2-yl)methyl]-2-(methylsulfanyl)pyrazolo[1,5-a][1,3,5]triazin-4-amine (Intermediate 116, 826 mg, 1.97 mmol) was suspended in dichloromethane (10 mL). To the ice-bath cooled suspension was added in portions 3-chlorobenzene-1-carboperoxoic acid (1.32 g, 77% purity, 5.90 mmol; CAS-RN:[937-14-4]). The reaction mixture was stirred over night at rt. Aqueous saturated sodium bicarbonate solution was added and the organic layer was separated. The aqueous layer was extracted with dichloromethane. The combined organic layers were dried over sodiumsulfate, filtrated and concentrated to give 760 mg (89% yield) of the title compound mixture.

LC-MS (Method 2): Rₜ=0.73 min; MS (ESIpos): m/z=434/450 [M+H]⁺

### Intermediate 118

### 8-bromo-N-[(5-chloro-1H-benzimidazol-2-yl)methyl]-2-(methylsulfanyl)pyrazolo[1,5-a][1,3,5]triazin-4-amine

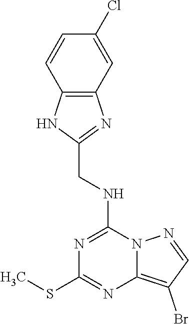 [SMILES: CSc1nc(NCc2nc3cc(Cl)ccc3[nH]2)n2ncc(Br)c2n1]

To dichloromethane (20 mL) was added 1-(5-chloro-1H-benzimidazol-2-yl)methanamine-hydrogen chloride (1/2) (500 mg, 1.96 mmol, CAS-RN:[1185297-00-0]), N,N-diisopropylethylamine (1.0 mL, 5.9 mmol; CAS-RN:[7087-68-5]) and 8-bromo-4-chloro-2-(methylsulfanyl)pyrazolo[1,5-a][1,3,5]triazine (Intermediate 7, 549 mg, 1.96 mmol). The reaction mixture was stirred over night at rt. The mixture was concentrated under reduced pressure and water was added to the residue. The precipitate was filtered off, washed with water and ethanol and dried at 60° C. under reduced pressure to give 730 mg (58% yield) of the title compound.

LC-MS (Method 2): Rₜ=1.20 min; MS (ESIpos): m/z=424 [M+H]⁺

¹H-NMR (400 MHz, DMSO-d6) δ[ppm]: 1.231 (0.68), 1.245 (2.27), 1.249 (1.48), 1.262 (3.57), 1.279 (2.01), 1.990 (0.65), 2.334 (0.68), 2.392 (0.85), 2.421 (16.00), 2.450 (0.79), 2.521 (3.35), 2.525 (2.19), 2.545 (0.45), 2.573 (6.92), 2.640 (0.72), 2.672 (1.04), 2.676 (0.71), 3.124 (0.47), 3.134 (0.49), 3.142 (0.51), 3.152 (0.51), 3.610 (0.41), 3.620 (0.40), 4.948 (2.94), 4.963 (2.90), 7.202 (1.41), 7.207 (1.42), 7.224 (1.62), 7.229 (1.64), 7.510 (1.91), 7.532 (1.66), 7.584 (1.93), 7.587 (1.86), 8.123 (2.65), 8.299 (7.45), 9.588 (0.76), 9.603 (1.53), 9.618 (0.69).

### Intermediate 119

### 8-bromo-N-[(5-chloro-1H-benzimidazol-2-yl)methyl]-2-methylsulfinyl-pyrazolo[1,5-a][1,3,5]triazin-4-amine and 8-bromo-N-[(5-chloro-1H-benzimidazol-2-yl)methyl]-2-methylsulfonyl-pyrazolo[1,5-a][1,3,5]triazin-4-amine

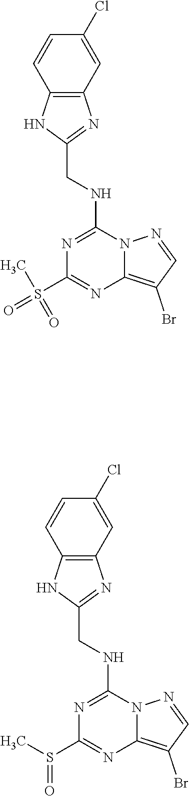 [SMILES: CS(=O)(=O)c1nc(NCc2nc3cc(Cl)ccc3[nH]2)n2ncc(Br)c2n1 ; CS(=O)c1nc(NCc2nc3cc(Cl)ccc3[nH]2)n2ncc(Br)c2n1]

8-bromo-N-[(5-chloro-1H-benzimidazol-2-yl)methyl]-2-(methylsulfanyl)pyrazolo[1,5-a][1,3,5]triazin-4-amine (Intermediate 118, 730 mg, 1.72 mmol) was suspended in dichloromethane (10 mL). To the ice-bath cooled suspension was added in portions 3-chlorobenzene-1-carboperoxoic acid (1.16 g, 77% purity, 5.16 mmol; CAS-RN:[937-14-4]). The reaction mixture was stirred over night at rt. Saturated sodium bicarbonate solution was added and the organic layer was separated. The aqueous layer was extracted with dichloromethane. The combined organic layers were dried over sodiumsulfate, filtrated and concentrated to give 636 mg (81% yield) of the title compound mixture.

LC-MS (Method 2): Rₜ=0.76/0.79 min; MS (ESIpos): m/z=440/456 [M+H]⁺

¹H-NMR (400 MHz, DMSO-d6) δ[ppm]: 1.193 (0.59), 1.209 (0.64), 1.217 (0.45), 1.240 (1.83), 1.257 (1.68), 2.289 (0.52), 2.339 (0.46), 2.521 (5.40), 2.525 (3.52), 2.540 (0.41), 2.681 (0.52), 2.742 (5.06), 2.855 (0.74), 3.257 (4.29), 3.283 (1.35), 3.364 (0.42), 4.993 (1.03), 5.056 (0.74), 5.761 (16.00), 7.179 (0.76), 7.196 (0.57), 7.412 (0.52), 7.431 (0.67), 7.456 (0.56), 7.477 (0.41), 7.528 (1.40), 7.548 (3.15), 7.555 (0.67), 7.562 (0.56), 7.569 (2.03), 7.576 (0.53), 7.619 (0.97), 7.639 (0.89), 7.659 (0.47), 7.695 (1.03), 7.698 (1.11), 7.701 (1.12), 7.704 (1.15), 7.716 (0.89), 7.718 (0.87), 7.720 (1.04), 7.724 (0.91), 7.820 (0.41), 7.825 (0.53), 7.831 (0.57), 7.835 (0.55), 7.837 (0.57), 7.840 (0.62), 7.854 (0.51), 7.860 (0.60), 7.867 (0.62), 7.872 (0.63), 7.883 (0.91), 7.887 (1.80), 7.890 (1.42), 7.894 (1.99), 7.898 (2.23), 7.904 (3.33), 7.906 (1.72), 8.183 (0.49), 8.206 (1.20), 8.486 (0.77), 8.505 (0.69), 8.509 (5.62), 8.586 (2.52), 10.094 (0.52), 12.484 (0.53).

### Intermediate 120

### 8-cyclopropyl-N-[(5-fluoro-1H-benzimidazol-2-yl)methyl]-2-(methylsulfanyl)pyrazolo[1,5-a][1,3,5]triazin-4-amine

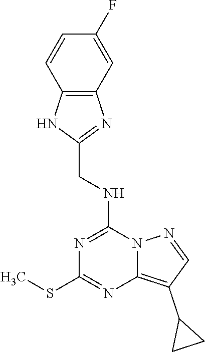 [SMILES: CSc1nc(NCc2nc3cc(F)ccc3[nH]2)n2ncc(C3CC3)c2n1]

To dichloromethane (20 mL) was added 1-(5-fluoro-1H-benzimidazol-2-yl)methanamine hydrogen chloride (1/2) (495 mg, 2.08 mmol, CAS-RN:[1216862-84-8]), N,N-diisopropylethylamine (1.1 mL, 6.2 mmol; CAS-RN:[7087-68-5]) and 4-chloro-8-cyclopropyl-2-(methylsulfanyl)pyrazolo[1,5-a][1,3,5]triazine (Intermediate 55, 500 mg, 2.08 mmol). The reaction mixture was stirred over night at rt. The mixture was concentrated under reduced pressure and treated with water. The precipitate was filtered off, washed with water and dried at 60° C. under reduced pressure to give 836 mg (98% yield) of the title compound.

LC-MS (Method 2): Rₜ=1.21 min; MS (ESIpos): m/z=370 [M+H]⁺

¹H-NMR (400 MHz, DMSO-d6) δ[ppm]: 0.811 (0.77), 0.824 (2.15), 0.829 (2.41), 0.836 (3.58), 0.842 (2.33), 0.850 (1.40), 0.854 (0.69), 0.865 (1.92), 0.872 (2.31), 0.878 (1.65), 0.881 (0.83), 0.886 (1.48), 0.893 (2.35), 0.899 (1.53), 0.911 (0.64), 1.231 (1.15), 1.245 (4.19), 1.249 (2.74), 1.262 (6.61), 1.268 (1.63), 1.280 (3.70), 1.845 (0.40), 1.857 (0.76), 1.865 (0.75), 1.874 (0.70), 1.878 (1.18), 1.886 (0.46), 1.891 (0.70), 1.899 (0.69), 2.406 (16.00), 2.469 (0.58), 2.521 (1.95), 2.526 (1.31), 3.124 (0.51), 3.134 (0.52), 3.142 (0.51), 3.153 (0.51), 3.610 (0.40), 4.879 (2.95), 4.894 (2.94), 6.973 (0.51), 6.980 (0.57), 6.998 (0.80), 7.002 (0.87), 7.020 (0.58), 7.026 (0.61), 7.936 (6.42), 9.241 (0.65), 9.257 (1.40), 9.272 (0.63).

### Intermediate 121

### 8-cyclopropyl-N-[(5-fluoro-1H-benzimidazol-2-yl)methyl]-2-(methanesulfonyl)pyrazolo[1,5-a][1,3,5]triazin-4-amine

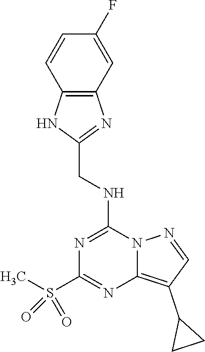 [SMILES: CS(=O)(=O)c1nc(NCc2nc3cc(F)ccc3[nH]2)n2ncc(C3CC3)c2n1]

8-cyclopropyl-N-[(5-fluoro-1H-benzimidazol-2-yl)methyl]-2-(methylsulfanyl)pyrazolo[1,5-a][1,3,5]triazin-4-amine (Intermediate 120, 835 mg, 2.26 mmol) was suspended in dichloromethane (20 mL). To the ice-bath cooled suspension was added in portions 3-chlorobenzene-1-carboperoxoic acid (1.52 g, 77% purity, 6.78 mmol; CAS-RN:[937-14-4]). The reaction mixture was stirred over night at rt. Aqueous saturated sodium bicarbonate solution was added and the organic layers were separated. The aqueous layer was extracted with dichloromethane. The combined organic layer was dried over sodiumsulfate, filtrated and concentrated to give 842 mg (93% yield) of the title compound.

LC-MS (Method 2): Rₜ=0.96 min; MS (ESIpos): m/z=402 [M+H]⁺

### Intermediate 122

### 8-cyclopropyl-N-[(4-fluoro-1H-benzimidazol-2-yl)methyl]-2-(methylsulfanyl)pyrazolo[1,5-a][1,3,5]triazin-4-amine

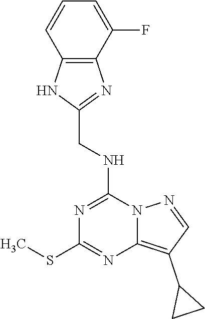 [SMILES: CSc1nc(NCc2nc3c(F)cccc3[nH]2)n2ncc(C3CC3)c2n1]

To dichloromethane (20 mL) was added 1-(4-fluoro-1H-benzimidazol-2-yl)methanamine hydrogen chloride (1/2) (618 mg, 80% purity, 2.08 mmol), N,N-diisopropylethylamine (1.4 mL, 8.3 mmol; CAS-RN:[7087-68-5]) and 4-chloro-8-cyclopropyl-2-(methylsulfanyl)pyrazolo[1,5-a][1,3,5]triazine (Intermediate 55, 500 mg, 2.08 mmol). The reaction mixture was stirred over night at rt. The mixture was concentrated under reduced pressure and treated with water. The precipitate was filtered off, washed with water and dried at 60° C. under reduced pressure to give 832 mg (crude) of the title compound.

LC-MS (Method 2): Rₜ=1.20 min; MS (ESIpos): m/z=370 [M+H]⁺

¹H-NMR (400 MHz, DMSO-d6) δ[ppm]: 0.814 (0.84), 0.828 (2.41), 0.833 (2.39), 0.840 (3.29), 0.845 (2.45), 0.853 (1.65), 0.866 (2.22), 0.872 (2.73), 0.878 (1.91), 0.886 (1.80), 0.893 (2.83), 0.899 (1.86), 0.911 (1.00), 1.234 (1.55), 1.247 (4.65), 1.264 (6.61), 1.282 (3.88), 1.846 (0.46), 1.859 (0.85), 1.867 (0.84), 1.876 (0.81), 1.880 (1.29), 1.888 (0.50), 1.893 (0.79), 1.901 (0.74), 2.374 (1.44), 2.407 (16.00), 2.521 (2.26), 2.526 (1.46), 2.585 (1.42), 3.121 (0.56), 3.132 (0.59), 3.139 (0.60), 3.150 (0.53), 3.607 (0.52), 3.618 (0.50), 4.901 (2.71), 4.909 (1.26), 4.916 (2.79), 6.923 (0.79), 6.942 (1.02), 6.951 (0.80), 6.969 (0.96), 7.106 (0.77), 7.118 (0.68), 7.127 (1.55), 7.139 (1.33), 7.147 (0.87), 7.159 (0.73), 7.241 (2.10), 7.261 (1.55), 7.383 (0.40), 7.852 (0.50), 7.933 (1.20), 7.942 (5.62), 8.101 (0.63), 9.282 (0.59), 9.298 (1.27), 9.313 (0.57), 12.604 (1.08).

### Intermediate 123

### 8-cyclopropyl-N-[(4-fluoro-1H-benzimidazol-2-yl)methyl]-2-(methanesulfonyl)pyrazolo[1,5-a][1,3,5]triazin-4-amine

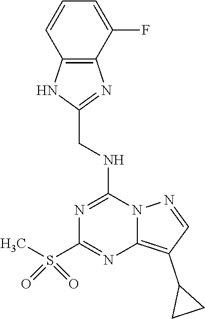 [SMILES: CS(=O)(=O)c1nc(NCc2nc3c(F)cccc3[nH]2)n2ncc(C3CC3)c2n1]

8-cyclopropyl-N-[(4-fluoro-1H-benzimidazol-2-yl)methyl]-2-(methylsulfanyl)pyrazolo[1,5-a][1,3,5]triazin-4-amine (Intermediate 122, 830 mg, 2.25 mmol) was suspended in dichloromethane (20 mL). To the ice-bath cooled suspension was added in portions 3-chlorobenzene-1-carboperoxoic acid (1.51 g, 77% purity, 6.74 mmol; CAS-RN:[937-14-4]). The reaction mixture was stirred over night at rt. Aqueous saturated sodium bicarbonate solution was added and the organic layer was separated. The aqueous layer was extracted with dichloromethane. The combined organic layers were dried over sodiumsulfate, filtrated and concentrated to give 880 mg of the title compound as a mixture with the corresponding sulfoxide.

LC-MS (Method 2): Rₜ=0.93 min; MS (ESIpos): m/z=402 [M+H]⁺

### Intermediate 124

### 8-iodo-2-(methylsulfanyl)pyrazolo[1,5-a][1,3,5]triazin-4-ol

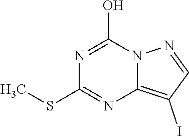

To a solution of 2-(methylsulfanyl)pyrazolo[1,5-a][1,3,5]triazin-4-ol (Intermediate 5, 2.00 g, 11.0 mmol) in N,N-dimethylformamide (25 mL) was added a solution of N-iodosuccinimide (2.59 g, 11.5 mmol; CAS-RN:[516-12-1]) in N,N-dimethylformamide (5 mL) at 0° C. The reaction mixture was stirred at 0° C. for 1 h. The mixture was poured into water. The resulting precipitate was filtered off and the solid was dried under vacuum to give 3.00 g (89% yield) of the title compound as a yellow solid.

### Intermediate 125

### 4-chloro-8-iodo-2-(methylsulfanyl)pyrazolo[1,5-a][1,3,5]triazine

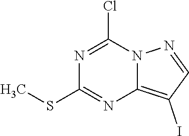

To a mixture of 8-iodo-2-(methylsulfanyl)pyrazolo[1,5-a][1,3,5]triazin-4-ol (Intermediate 124, 7.00 g, 22.7 mmol) in phosphorus oxychloride (70 mL) was added N,N-dimethylaniline (8.6 mL, 68 mmol; CAS-RN:[121-69-7]) in one portion. The reaction mixture was stirred at 100° C. for 2 h. The reaction mixture was concentrated under reduced pressure to give a residue. The residue was diluted with water and extracted with ethyl acetate. The organic phase was washed with brine, dried over anhydrous sodium sulfate and filtered. The filtrate was concentrated and purified by column chromatography on silica gel (200-300 mesh, petroleum ether: ethyl acetate=40:1) to give 6.00 g (81% yield) of the title compound as a yellow solid.

### Intermediate 126

### 8-iodo-N,N-bis[(4-methoxyphenyl)methyl]-2-(methylsulfanyl)pyrazolo[1,5-a][1,3,5]triazin-4-amine

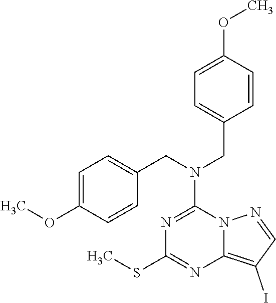 [SMILES: COc1ccc(CN(Cc2ccc(OC)cc2)c2nc(SC)nc3c(I)cnn23)cc1]

To a mixture of 4-chloro-8-iodo-2-(methylsulfanyl)pyrazolo[1,5-a][1,3,5]triazine (Intermediate 125, 3.00 g, 9.19 mmol) and 1-(4-methoxyphenyl)-N-[(4-methoxyphenyl)methyl]methanamine (2.84 g, 11.0 mmol, CAS-RN:[17061-62-0]) in tetrahydrofuran (20 mL) was added N,N-diisopropylethylamine (4.8 mL, 28 mmol; CAS-RN:[7087-68-5]) at rt. The mixture was stirred at rt for 2 h. The reaction mixture was concentrated under reduced pressure to give a residue. The residue was diluted with water and extracted with ethyl acetate. The organic phase was washed with brine, dried with anhydrous sodium sulfate and filtered. The filtrate was concentrated in reduced pressure to give 2.00 g (40% yield) of the title compound as a white solid.

LC-MS (Method D): Rₜ=1.075 min; MS (ESIpos): m/z=548.1 [M+H]⁺.

### Intermediate 127

### 8-iodo-2-(methanesulfonyl)-N,N-bis[(4-methoxyphenyl)methyl]pyrazolo[1,5-a][1,3,5]triazin-4-amine

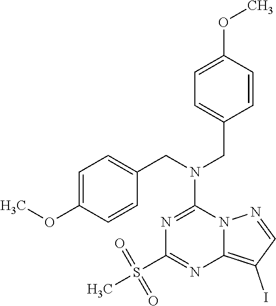 [SMILES: COc1ccc(CN(Cc2ccc(OC)cc2)c2nc(S(C)(=O)=O)nc3c(I)cnn23)cc1]

To a solution of 8-iodo-N,N-bis[(4-methoxyphenyl)methyl]-2-(methylsulfanyl)pyrazolo[1,5-a][1,3,5]triazin-4-amine (Intermediate 126, 2.00 g, 3.65 mmol) in dichloromethane (15 mL) was added meta-Chloroperoxybenzoic acid (1.89 g, 11.0 mmol; CAS-RN:[937-14-4]) at 0° C. The reaction mixture was stirred at rt for 12 h. The mixture was diluted with dichloromethane and washed with saturated sodium bicarbonate and brine. The organic phase was dried over anhydrous sodium sulfate, filtered and the filtrate was concentrated to give 3.30 g (crude) of the title compound as a white solid.

LC-MS (Method D): Rₜ=1.033 min; MS (ESIpos): m/z=580.3 [M+H]⁺.

### Intermediate 128

### 8-iodo-N, N-bis[(4-methoxyphenyl)methyl]-2-(morpholin-4-yl)pyrazolo[1,5-a][1,3,5]triazin-4-amine

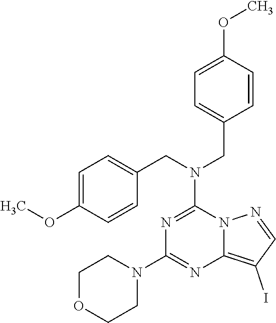 [SMILES: COc1ccc(CN(Cc2ccc(OC)cc2)c2nc(N3CCOCC3)nc3c(I)cnn23)cc1]

To a solution of 8-iodo-2-(methanesulfonyl)-N,N-bis[(4-methoxyphenyl)methyl]pyrazolo[1,5-a][1,3,5]triazin-4-amine (Intermediate 127, 3.30 g, 60% purity, 3.42 mmol) and morpholine (893 mg, 10.3 mmol) in acetonitrile (20 mL) was added N,N-diisopropylethylamine (1.8 mL, 10 mmol; CAS-RN:[7087-68-5]) at rt. The reaction mixture was stirred at 70° C. for 16 h. The reaction mixture was filtered and the filter cake was dried to give 2.40 g (crude) of the title compound as am off-white solid.

LC-MS (Method D): Rₜ=1.142 min; MS (ESIpos): m/z=587.3 [M+H]⁺.

### Intermediate 129

### N,N-bis[(4-methoxyphenyl)methyl]-2-(morpholin-4-yl)-8-(trifluoromethyl)pyrazolo[1,5-a][1,3,5]triazin-4-amine

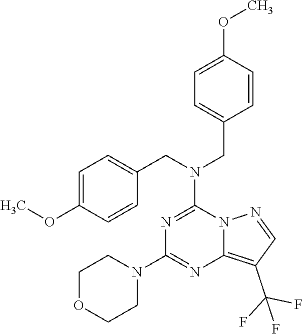 [SMILES: COc1ccc(CN(Cc2ccc(OC)cc2)c2nc(N3CCOCC3)nc3c(C(F)(F)F)cnn23)cc1]

To a solution of 8-iodo-N,N-bis[(4-methoxyphenyl)methyl]-2-(morpholin-4-yl)pyrazolo[1,5-a][1,3,5]triazin-4-amine (Intermediate 128, 820 mg, 1.40 mmol) in N,N-dimethylformamide (40 mL) were added methyl difluoro(fluorosulfonyl)acetate (1.07 g, 5.59 mmol) and copper(I) iodide (1.07 g, 5.59 mmol) at rt. The reaction mixture was stirred at 80° C. for 16 h. The reaction mixture was filtered. The filtrate was concentrated and purified by column chromatography on silica gel (200-300 mesh, petroleum ether: ethyl acetate=50:1) to give 650 mg (88% yield) of the title compound as a white solid.

LC-MS (Method D): Rₜ=1.139 min; MS (ESIpos): m/z=529.4 [M+H]⁺.

### Intermediate 130

### 2-(chloromethyl)-1-{[2-(trimethylsilyl)ethoxy]methyl}-1H-benzimidazole

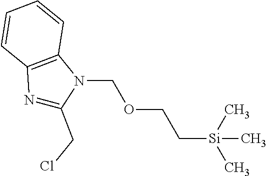 [SMILES: C[Si](C)(C)CCOCn1c(CCl)nc2ccccc21]

To a solution of 2-(chloromethyl)-1H-benzimidazole (500 mg, 3.00 mmol; CAS-RN:[4857-04-9]) in tetrahydrofuran (5.0 mL) were added N,N-diisopropylethylamine (1.0 mL, 6.0 mmol; CAS-RN:[7087-68-5]) and 2-(trimethylsilyl)ethoxymethyl chloride (830 μL, 3.6 mmol; CAS-RN:[76513-69-4]) at rt. The reaction mixture was stirred at 25° C. for 1 h. The mixture was concentrated in vacuum to give a residue. The residue was purified by silica gel chromatography (1000 mesh, petroleum ether: ethyl acetate=10:1 to 4:1) to give 200 mg (22% yield) of the title compound as a yellow oil.

LC-MS (Method D): Rₜ=0.964 min; MS (ESIpos): m/z=297.6 [M+H]⁺.

### Intermediate 131

### 2-(morpholin-4-yl)-8-(trifluoromethyl)pyrazolo[1,5-a][1,3,5]triazin-4-amine

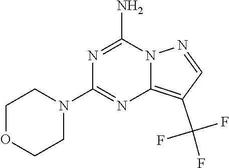 [SMILES: Nc1nc(N2CCOCC2)nc2c(C(F)(F)F)cnn12]

A solution of N,N-bis[(4-methoxyphenyl)methyl]-2-(morpholin-4-yl)-8-(trifluoromethyl)pyrazolo[1,5-a][1,3,5]triazin-4-amine (Intermediate 129, 650 mg, 1.23 mmol) in trifluoromethanesulfonic acid (23 mL) was stirred at 70° C. for 16 h. The reaction mixture was poured into ice water and basified to pH=8 by sodium carbonate. The mixture was extracted with ethyl acetate. The organic phase was washed with brine, dried over anhydrous sodium sulfate and filtered. The filtrate was concentrated and purified by column chromatography on silica gel (200-300 mesh, petroleum ether: ethyl acetate=5:1) to give 400 mg (crude) of the title compound as a yellow solid.

LC-MS (Method D): Rₜ=0.856 min; MS (ESIpos): m/z=289.5 [M+H]⁺.

### Intermediate 132

### 2-(morpholin-4-yl)-8-(trifluoromethyl)-N-[(1-{[2-(trimethylsilyl)ethoxy]methyl}-1H-benzimidazol-2-yl)methyl]pyrazolo[1,5-a][1,3,5]triazin-4-amine

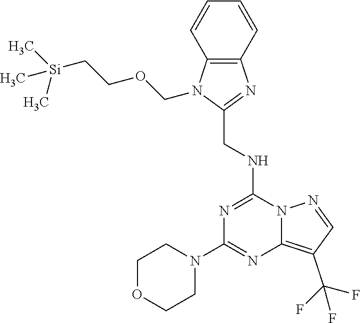 [SMILES: C[Si](C)(C)CCOCn1c(CNc2nc(N3CCOCC3)nc3c(C(F)(F)F)cnn23)nc2ccccc21]

To a solution of 2-(morpholin-4-yl)-8-(trifluoromethyl)pyrazolo[1,5-a][1,3,5]triazin-4-amine (Intermediate 131, 100 mg, 84% purity, 291 μmol) in N,N-dimethylformamide (10 mL) was added sodium hydride (17.5 mg, 60% purity, 437 μmol; CAS-RN:[7646-69-7]) at rt. After stirring at 60° C. for 10 min, 2-(chloromethyl)-1-{[2-(trimethylsilyl)ethoxy]methyl}-1H-benzimidazole (Intermediate 130, 130 mg, 437 μmol) was added in one portion. The reaction mixture was stirred at 60° C. for 2 h. The reaction mixture was concentrated under reduced pressure to give a residue. The residue was diluted with water and extracted with ethyl acetate. The organic phase was washed with brine, dried over anhydrous sodium sulfate and filtered. The filtrate was concentrated and purified by column chromatography on silica gel (200-300 mesh, petroleum ether: ethyl acetate=5:1) to give 200 mg (78% purity, 98% yield) of the title compound as a yellow oil.

LC-MS (Method D): Rₜ=0.964 min; MS (ESIpos): m/z=549.3 [M+H]⁺.

### Intermediate 133

### 8-bromo-N-[(4-methoxyphenyl)methyl]-N-({1-[(4-methoxyphenyl)methyl]-1H-benzimidazol-2-yl}methyl)-2-(4-methylpiperazin-1-yl)pyrazolo[1,5-a][1,3,5]triazin-4-amine

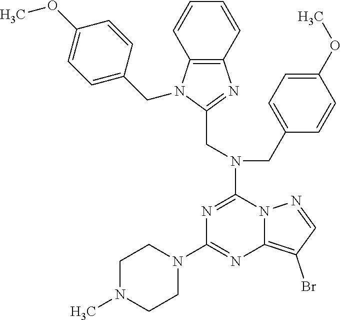 [SMILES: COc1ccc(CN(Cc2nc3ccccc3n2Cc2ccc(OC)cc2)c2nc(N3CCN(C)CC3)nc3c(Br)cnn23)cc1]

8-bromo-2-(methanesulfonyl)-N-[(4-methoxyphenyl)methyl]-N-({1-[(4-methoxyphenyl)methyl]-1H-benzimidazol-2-yl}methyl)pyrazolo[1,5-a][1,3,5]triazin-4-amine (Intermediate 60, 2.60 g, 3.92 mmol) was dissolved in acetonitrile (56 mL), 1-methylpiperazine (904 mg, 9.03 mmol; CAS-RN:[109-01-3]) was added and the reaction mixture was stirred for 2 h at 50° C. and overnight at rt. The suspension was treated with water, the precipitate was filtered off, washed with water and dried to give 2.48 g (93% yield) of the title compound.

LC-MS (Method 1): Rₜ=1.15 min; MS (ESIpos): m/z=682 [M+H]⁺

¹H-NMR (400 MHz, DMSO-d6) δ[ppm]: 2.158 (2.29), 2.198 (0.47), 2.242 (0.53), 2.325 (0.58), 2.329 (0.72), 2.334 (0.54), 2.520 (2.15), 2.525 (1.48), 2.667 (0.42), 2.671 (0.60), 2.676 (0.41), 3.603 (0.41), 3.651 (0.62), 3.670 (0.54), 3.694 (5.45), 3.712 (0.74), 3.723 (16.00), 5.413 (2.45), 6.828 (0.52), 6.857 (0.48), 6.864 (3.05), 6.869 (1.04), 6.881 (1.06), 6.886 (3.20), 6.893 (0.42), 7.007 (0.47), 7.163 (0.65), 7.179 (1.05), 7.194 (0.66), 7.248 (1.28), 7.269 (1.13), 7.489 (0.49), 7.507 (0.44), 7.589 (0.53), 7.607 (0.48).

### Intermediate 134

### N-[(4-methoxyphenyl)methyl]-N-({1-[(4-methoxyphenyl)methyl]-1H-benzimidazol-2-yl}methyl)-2-(4-methylpiperazin-1-yl)-8-(pyridin-4-yl)pyrazolo[1,5-a][1,3,5]triazin-4-amine

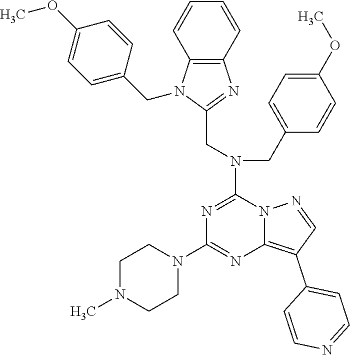 [SMILES: COc1ccc(CN(Cc2nc3ccccc3n2Cc2ccc(OC)cc2)c2nc(N3CCN(C)CC3)nc3c(-c4ccncc4)cnn23)cc1]

8-bromo-N-[(4-methoxyphenyl)methyl]-N-({1-[(4-methoxyphenyl)methyl]-1H-benzimidazol-2-yl}methyl)-2-(4-methylpiperazin-1-yl)pyrazolo[1,5-a][1,3,5]triazin-4-amine (Intermediate 133, 250 mg, 366 μmol) and 4-(4,4,5,5-tetramethyl-1,3,2-dioxaborolan-2-yl)pyridine (113 mg, 549 μmol; CAS-RN:[181219-01-2]) were provided in tetrahydrofurane (3.8 mL), potassium phosphate (272 mg, 1.28 mmol; CAS-RN:[7778-53-2]) and water (770 μL) were added and the mixture was flushed with argon for 5 min. [1,1′-Bis(diphenylphosphino)ferrocene]dichloropalladium(II) dichloromethane complex (19.5 mg, 23.9 μmol; CAS-RN:[95464-05-4]) was added under argon and the mixture was stirred for 1 h at 130° C. in a microwave. The mixture was concentrated and purified by flash chromatography using silica gel (dichloromethane-ethanol gradient) to give 162 mg (65% yield) of the title compound.

LC-MS (Method 2): Rₜ=1.40 min; MS (ESIpos): m/z=681 [M+H]⁺

¹H-NMR (400 MHz, DMSO-d6) δ[ppm]: 0.800 (0.49), 0.817 (0.53), 0.824 (0.53), 0.907 (0.60), 1.068 (0.65), 2.179 (1.85), 2.220 (0.46), 2.320 (0.53), 2.325 (0.79), 2.329 (1.01), 2.334 (0.81), 2.338 (0.52), 2.520 (3.13), 2.525 (2.08), 2.667 (0.50), 2.671 (0.72), 2.676 (0.52), 3.654 (0.97), 3.698 (0.76), 3.716 (1.09), 3.727 (16.00), 5.441 (2.39), 5.761 (2.34), 6.868 (0.48), 6.876 (2.86), 6.881 (1.03), 6.893 (1.02), 6.898 (3.02), 6.905 (0.42), 7.160 (0.56), 7.179 (0.91), 7.196 (0.60), 7.281 (1.22), 7.301 (1.10), 7.496 (0.48), 7.514 (0.45), 7.591 (0.46), 7.607 (0.42), 7.939 (2.58), 7.943 (1.53), 7.951 (1.57), 7.955 (2.61), 8.456 (2.77), 8.460 (1.62), 8.468 (1.55), 8.472 (2.40).

### Intermediate 135

### N-[(4-methoxyphenyl)methyl]-N-({1-[(4-methoxyphenyl)methyl]-1H-benzimidazol-2-yl}methyl)-2-(4-methylpiperazin-1-yl)-8-(2-methylpyridin-4-yl)pyrazolo[1,5-a][1,3,5]triazin-4-amine

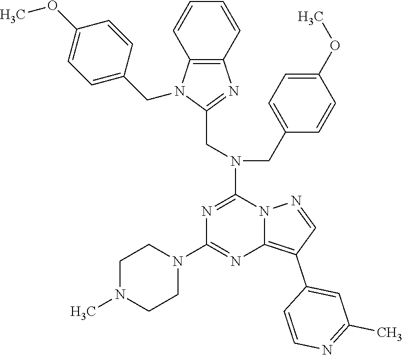

8-bromo-N-[(4-methoxyphenyl)methyl]-N-({1-[(4-methoxyphenyl)methyl]-1H-benzimidazol-2-yl}methyl)-2-(4-methylpiperazin-1-yl)pyrazolo[1,5-a][1,3,5]triazin-4-amine (Intermediate 133, 250 mg, 366 μmol) and 2-methyl-4-(4,4,5,5-tetramethyl-1,3,2-dioxaborolan-2-yl)pyridine (120 mg, 549 μmol, CAS-RN:[660867-80-1]) were provided in tetrahydrofurane (3.8 mL), potassium phosphate (272 mg, 1.28 mmol; CAS-RN:[7778-53-2]) and water (770 μL) were added and the mixture was flushed with argon for 5 min. [1,1′-Bis(diphenylphosphino)ferrocene]dichloropalladium(II) dichloromethane complex (19.5 mg, 23.9 μmol; CAS-RN:[95464-05-4]) was added under argon and the mixture was stirred for 1 h at 130° C. in a microwave. The mixture was concentrated and purified by flash chromatography using silica gel (dichloromethane-ethanol gradient) to give 198 mg (78% yield) of the title compound.

LC-MS (Method 2): Rₜ=1.43 min; MS (ESIpos): m/z=695 [M+H]⁺

¹H-NMR (400 MHz, DMSO-d6) δ[ppm]: 1.038 (0.44), 1.055 (0.96), 1.068 (16.00), 1.158 (5.30), 2.177 (1.41), 2.320 (0.44), 2.325 (0.65), 2.329 (0.82), 2.334 (0.65), 2.338 (0.41), 2.444 (5.34), 2.520 (2.46), 2.525 (1.63), 2.667 (0.42), 2.671 (0.60), 2.676 (0.43), 3.659 (0.89), 3.698 (0.80), 3.716 (0.94), 3.727 (10.93), 3.940 (2.33), 5.441 (1.84), 5.761 (2.57), 6.866 (0.48), 6.874 (2.13), 6.879 (0.79), 6.891 (0.80), 6.896 (2.21), 7.160 (0.46), 7.178 (0.73), 7.197 (0.48), 7.276 (0.97), 7.297 (0.85), 7.778 (1.17), 7.805 (0.67), 7.819 (0.67), 8.332 (1.06), 8.345 (1.00).

### Intermediate 136

### 8-(cyclopent-1-en-1-yl)-N,N-bis[(4-methoxyphenyl)methyl]-2-(morpholin-4-yl)pyrazolo[1,5-a][1,3,5]triazin-4-amine

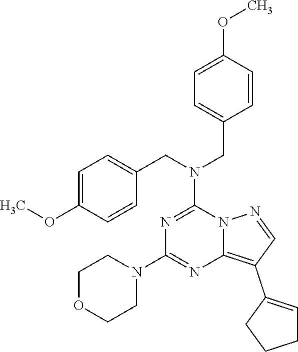 [SMILES: COc1ccc(CN(Cc2ccc(OC)cc2)c2nc(N3CCOCC3)nc3c(C4=CCCC4)cnn23)cc1]

To a mixture of 8-iodo-N,N-bis[(4-methoxyphenyl)methyl]-2-(morpholin-4-yl)pyrazolo[1,5-a][1,3,5]triazin-4-amine (Intermediate 128, 1.00 g, 1.71 mmol) and cyclopent-1-en-1-ylboronic acid (573 mg, 5.12 mmol, CAS-RN:[850036-28-1]) in tetrahydrofuran (12 mL) were added methanesulfonato(2-dicyclohexylphosphino-2′,4′,6′-tri-i-propyl-1,1′-biphenyl)(2′-amino-1,1′-biphenyl-2-yl)palladium(II) (144 mg, 171 μmol; CAS-RN:[1445085-55-1]) and potassium phosphate (1.36 g, 6.4 mmol) in one portion. The reaction mixture was stirred at 60° C. for 4 h under nitrogen atmosphere. The reaction mixture was concentrated under reduced pressure to give a residue. The residue was diluted with water and extracted with ethyl acetate. The organic phase was washed with brine, dried over anhydrous sodium sulfate and filtered. The filtrate was concentrated and purified by column chromatography on silica gel (200-300 mesh, petroleum ether: ethyl acetate=10:1) to give 530 mg (59% yield) of the title compound as a yellow solid.

LC-MS (Method D): Rₜ=1.200 min; MS (ESIpos): m/z=527.5 [M+H]⁺.

### Intermediate 137

### 8-cyclopentyl-N,N-bis[(4-methoxyphenyl)methyl]-2-(morpholin-4-yl)pyrazolo[1,5-a][1,3,5]triazin-4-amine

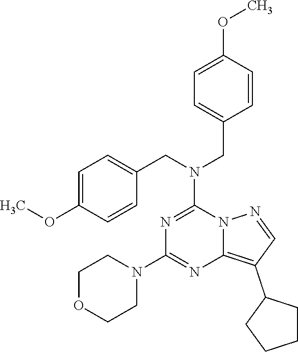 [SMILES: COc1ccc(CN(Cc2ccc(OC)cc2)c2nc(N3CCOCC3)nc3c(C4CCCC4)cnn23)cc1]

To a solution of 8-(cyclopent-1-en-1-yl)-N,N-bis[(4-methoxyphenyl)methyl]-2-(morpholin-4-yl)pyrazolo[1,5-a][1,3,5]triazin-4-amine (Intermediate 136, 430 mg, 816 μmol) in methanol was added palladium on carbon (20.0 mg, 10% purity). The reaction mixture was stirred at 40° C. for 16 h under hydrogen atmosphere. The reaction mixture was filtered and the filtrate was concentrated under reduced pressure to give 420 mg (97% yield) of the title compound as a yellow solid.

LC-MS (Method D): Rₜ=1.125 min; MS (ESIpos): m/z=529.5 [M+H]⁺.

### Intermediate 138

### 8-cyclopentyl-2-(morpholin-4-yl)pyrazolo[1,5-a][1,3,5]triazin-4-amine

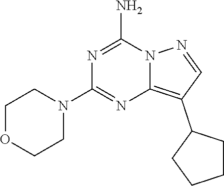 [SMILES: Nc1nc(N2CCOCC2)nc2c(C3CCCC3)cnn12]

A solution of 8-cyclopentyl-N,N-bis[(4-methoxyphenyl)methyl]-2-(morpholin-4-yl)pyrazolo[1,5-a][1,3,5]triazin-4-amine (Intermediate 137, 320 mg, 605 μmol) in trifluoromethanesulfonic acid (6.0 mL, 67.8 mmol) was stirred at 70° C. for 16 h. The reaction mixture was poured into ice water and basified to pH=8 by sodium carbonate. The mixture was extracted with ethyl acetate. The organic phase was washed with brine, dried over anhydrous sodium sulfate and filtered. The filtrate was concentrated and purified by column chromatography on silica gel (200-300 mesh, petroleum ether: ethyl acetate=5:1) to give 200 mg (crude) of the title compound as a yellow solid.

LC-MS (Method D): Rₜ=0.831 min; MS (ESIpos): m/z=289.6 [M+H]⁺.

### Intermediate 139

### 8-cyclopentyl-2-(morpholin-4-yl)-N-[(1-{[2-(trimethylsilyl)ethoxy]methyl}-1H-benzimidazol-2-yl)methyl]pyrazolo[1,5-a][1,3,5]triazin-4-amine

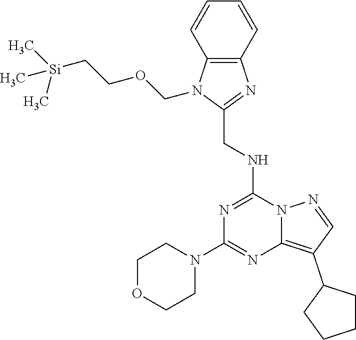 [SMILES: C[Si](C)(C)CCOCn1c(CNc2nc(N3CCOCC3)nc3c(C4CCCC4)cnn23)nc2ccccc21]

To a solution of 8-cyclopentyl-2-(morpholin-4-yl)pyrazolo[1,5-a][1,3,5]triazin-4-amine (Intermediate 138, 95.0 mg, 329 μmol) in N,N-dimethylformamide (2.0 mL) was added sodium hydride (19.8 mg, 60% purity, 494 μmol; CAS-RN:[7646-69-7]) at rt. After stirring at 60° C. for 15 min, 2-(chloromethyl)-1-{[2-(trimethylsilyl)ethoxy]methyl}-1H-benzimidazole (Intermediate 130, 127 mg, 428 μmol) was added in one portion. The reaction mixture was stirred at rt for 16 h. The reaction mixture was concentrated under reduced pressure to give a residue. The residue was diluted with water and extracted with ethyl acetate. The organic phase was washed with brine, dried over anhydrous sodium sulfate and filtered. The filtrate was concentrated to give 120 mg (66% yield) of the title compound as a yellow oil.

LC-MS (Method D): Rₜ=1.006 min; MS (ESIpos): m/z=549.4 [M+H]⁺.

### Intermediate 140

### tert-butyl [(3S)-1-(4-{[(1H-benzimidazol-2-yl)methyl]amino}-8-bromopyrazolo[1,5-a][1,3,5]triazin-2-yl)pyrrolidin-3-yl]carbamate

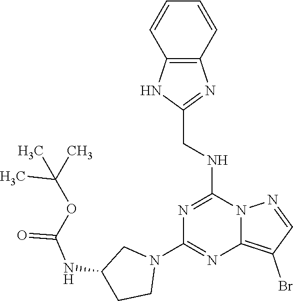 [SMILES: CC(C)(C)OC(=O)N[C@H]1CCN(c2nc(NCc3nc4ccccc4[nH]3)n3ncc(Br)c3n2)C1]

To a solution of N-[(1H-benzimidazol-2-yl)methyl]-8-bromo-2-(methanesulfonyl)pyrazolo[1,5-a][1,3,5]triazin-4-amine (Intermediate 9, 200 mg, 45% purity, 213 μmol) in acetonitrile (3.0 mL) were added N,N-diisopropylethylamine (110 μL, 640 μmol; CAS-RN:[7087-68-5]) and tert-butyl (3S)-pyrrolidin-3-ylcarbamate (119 mg, 639 μmol, CAS-RN:[122536-76-9]). The reaction mixture was stirred at 70° C. for 19 h. The reaction was treated with water and was extracted with ethylacetate. The combined organic layers were concentrated under reduced pressure to give 254 mg (66% purity, crude) of the title compound which was used without further purification.

LC-MS (Method 2): Rₜ=1.19 min; MS (ESIpos): m/z=528 [M+H]⁺

### Intermediate 141

### tert-butyl [(3R)-1-(4-{[(1H-benzimidazol-2-yl)methyl]amino}-8-bromopyrazolo[1,5-a][1,3,5]triazin-2-yl)pyrrolidin-3-yl]carbamate

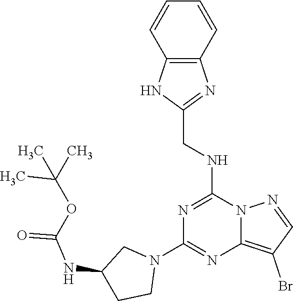 [SMILES: CC(C)(C)OC(=O)N[C@@H]1CCN(c2nc(NCc3nc4ccccc4[nH]3)n3ncc(Br)c3n2)C1]

To a solution of N-[(1H-benzimidazol-2-yl)methyl]-8-bromo-2-(methanesulfonyl)pyrazolo[1,5-a][1,3,5]triazin-4-amine (Intermediate 9, 200 mg, 45% purity, 213 μmol) in acetonitrile (3.0 mL) were added N,N-diisopropylethylamine (110 μL, 640 μmol; CAS-RN:[7087-68-5]) and tert-butyl (3R)-pyrrolidin-3-ylcarbamate (119 mg, 639 μmol, CAS-RN:[122536-77-0]). The reaction mixture was stirred at 70° C. for 18 h. The reaction was treated with water and was extracted three times with ethylacetate. The combined organic layer was concentrated under reduced pressure to give a residue, which was purified by flash chromatography (silica gel, dichloromethane/EtOH gradient) to give 126 mg (98% yield) of the title compound.

LC-MS (Method 2): Rₜ=1.19 min; MS (ESIpos): m/z=528 [M+H]⁺

### Intermediate 142

### tert-butyl [(7S)-5-(4-{[(1H-benzimidazol-2-yl)methyl]amino}-8-bromopyrazolo[1,5-a][1,3,5]triazin-2-yl)-5-azaspiro[2.4]heptan-7-yl]carbamate

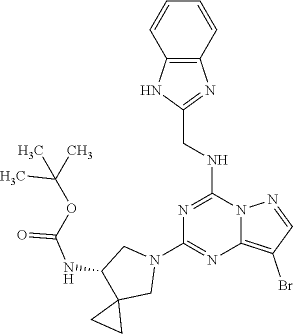 [SMILES: CC(C)(C)OC(=O)N[C@@H]1CN(c2nc(NCc3nc4ccccc4[nH]3)n3ncc(Br)c3n2)CC12CC2]

To a solution of N-[(1H-benzimidazol-2-yl)methyl]-8-bromo-2-(methanesulfonyl)pyrazolo[1,5-a][1,3,5]triazin-4-amine (Intermediate 9, 200 mg, 45% purity, 213 μmol) in acetonitrile (3.0 mL) were added N,N-diisopropylethylamine (110 μL, 640 μmol; CAS-RN:[7087-68-5]) and tert-butyl (7S)-5-azaspiro[2.4]heptan-7-ylcarbamate (136 mg, 639 μmol, CAS-RN:[127199-45-5]). The reaction mixture was stirred at 70° C. for 18 h. The reaction was treated with water and was extracted with ethylacetate. The combined organic layers were concentrated under reduced pressure to give a residue, which was purified by flash chromatography (silica gel, dichloromethane/EtOH gradient) to give 134 mg (100% yield) of the title compound.

LC-MS (Method 2): Rₜ=1.26 min; MS (ESIpos): m/z=554 [M+H]⁺

### Intermediate 143

### tert-butyl [(3S)-1-(4-{[(1H-benzimidazol-2-yl)methyl]amino}-8-bromopyrazolo[1,5-a][1,3,5]triazin-2-yl)pyrrolidin-3-yl]methylcarbamate

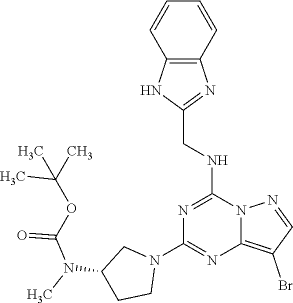 [SMILES: CN(C(=O)OC(C)(C)C)[C@H]1CCN(c2nc(NCc3nc4ccccc4[nH]3)n3ncc(Br)c3n2)C1]

To a solution of N-[(1H-benzimidazol-2-yl)methyl]-8-bromo-2-(methanesulfonyl)pyrazolo[1,5-a][1,3,5]triazin-4-amine (Intermediate 9, 200 mg, 45% purity, 213 μmol) in acetonitrile (3.0 mL) were added N,N-diisopropylethylamine (110 μL, 640 μmol; CAS-RN:[7087-68-5]) and tert-butyl methyl[(3S)-pyrrolidin-3-yl]carbamate (128 mg, 639 μmol, CAS-RN:[169750-01-0]). The reaction mixture was stirred at 70° C. for 23 h. The reaction was treated with water and was extracted with ethylacetate. The combined organic layer was concentrated under reduced pressure to give a residue, which was purified by flash chromatography (silica gel, dichloromethane/EtOH gradient) to give 138 mg of the title compound.

LC-MS (Method 2): Rₜ=1.28 min; MS (ESIneg): m/z=540 [M−H]⁻

### Intermediate 144

### tert-butyl [(3R)-1-(4-{[(1H-benzimidazol-2-yl)methyl]amino}-8-bromopyrazolo[1,5-a][1,3,5]triazin-2-yl)pyrrolidin-3-yl]methylcarbamate

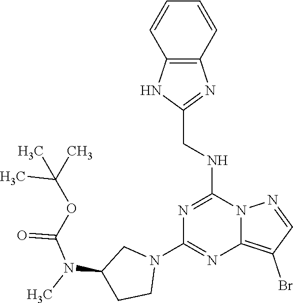 [SMILES: CN(C(=O)OC(C)(C)C)[C@@H]1CCN(c2nc(NCc3nc4ccccc4[nH]3)n3ncc(Br)c3n2)C1]

To a solution of N-[(1H-benzimidazol-2-yl)methyl]-8-bromo-2-(methanesulfonyl)pyrazolo[1,5-a][1,3,5]triazin-4-amine (Intermediate 9, 200 mg, 45% purity, 213 μmol) in acetonitrile (3.0 mL) were added N,N-diisopropylethylamine (110 μL, 640 μmol; CAS-RN:[7087-68-5]) and tert-butyl methyl[(3R)-pyrrolidin-3-yl]carbamate (128 mg, 639 μmol, CAS-RN:[392338-15-7]). The reaction mixture was stirred at 70° C. for 23 h. The reaction was treated with water and was extracted with ethylacetate. The combined organic layers were concentrated under reduced pressure to give a residue, which was purified by flash chromatography (silica gel, dichloromethane/EtOH gradient) to give 152 mg of the title compound.

LC-MS (Method 2): Rₜ=1.28 min; MS (ESIpos): m/z=542 [M+H]⁺

### Intermediate 145

### 8-cyclopropyl-N-[(5-methyl-1H-benzimidazol-2-yl)methyl]-2-(methylsulfanyl)pyrazolo[1,5-a][1,3,5]triazin-4-amine

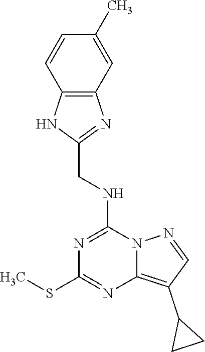 [SMILES: CSc1nc(NCc2nc3cc(C)ccc3[nH]2)n2ncc(C3CC3)c2n1]

To dichloromethane (30 mL) was added 1-(5-methyl-1H-benzimidazol-2-yl)methanamine hydrogen chloride (1/2) (973 mg, 4.15 mmol; CAS-RN:[89219-02-3]), N,N-diisopropylethylamine (2.9 mL, 17 mmol; CAS-RN:[7087-68-5]) and 4-chloro-8-cyclopropyl-2-(methylsulfanyl)pyrazolo[1,5-a][1,3,5]triazine (Intermediate 55, 1.00 g, 4.15 mmol). The reaction mixture was stirred over night at rt. The mixture was concentrated under reduced pressure and treated with water. The precipitate was filtered off, washed with water and dried at 60° C. under reduced pressure to give 1.78 g (crude) of the title compound.

LC-MS (Method 2): Rₜ=1.23 min; MS (ESIpos): m/z=366 [M+H]⁺

¹H-NMR (400 MHz, DMSO-d6) δ[ppm]: −0.009 (0.61), 0.008 (0.51), 0.810 (0.67), 0.824 (2.09), 0.829 (2.29), 0.836 (3.37), 0.842 (2.27), 0.849 (1.34), 0.853 (0.73), 0.864 (1.88), 0.871 (2.16), 0.876 (1.52), 0.881 (0.84), 0.885 (1.40), 0.892 (2.31), 0.897 (1.46), 0.909 (0.63), 1.258 (1.79), 1.857 (0.70), 1.864 (0.72), 1.874 (0.70), 1.877 (1.11), 1.886 (0.43), 1.890 (0.67), 1.898 (0.63), 2.382 (5.07), 2.410 (16.00), 2.520 (1.79), 2.525 (1.15), 4.860 (2.20), 4.875 (2.19), 6.935 (0.43), 6.955 (0.77), 7.193 (0.74), 7.325 (0.57), 7.391 (0.50), 7.411 (0.45), 7.930 (6.19), 9.217 (0.80), 12.122 (0.70), 12.140 (0.59).

### Intermediate 146

### 8-cyclopropyl-2-(methanesulfonyl)-N-[(5-methyl-1H-benzimidazol-2-yl)methyl]pyrazolo[1,5-a][1,3,5]triazin-4-amine; 8-cyclopropyl-N-[(5-methyl-1H-benzimidazol-2-yl)methyl]-2-methylsulfinyl-pyrazolo[1,5-a][1,3,5]triazin-4-amine

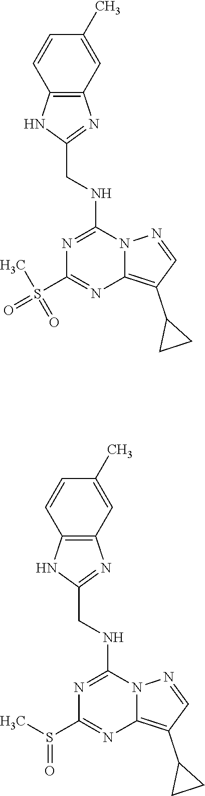 [SMILES: Cc1ccc2[nH]c(CNc3nc(S(C)(=O)=O)nc4c(C5CC5)cnn34)nc2c1 ; Cc1ccc2[nH]c(CNc3nc(S(C)=O)nc4c(C5CC5)cnn34)nc2c1]

8-cyclopropyl-N-[(5-methyl-1H-benzimidazol-2-yl)methyl]-2-(methylsulfanyl)pyrazolo[1,5-a][1,3,5]triazin-4-amine (Intermediate 145, 1.00 g, 2.74 mmol) was suspended in dichloromethane (10 mL). To the ice-bath cooled suspension was added in portions 3-chlorobenzene-1-carboperoxoic acid (1.84 g, 77% purity, 8.21 mmol; CAS-RN:[937-14-4]). The reaction mixture was stirred over night at rt. Aqueous saturated sodium bicarbonate solution was added and the organic layer was separated. The aqueous layer was extracted with dichloromethane. The combined organic layers were dried over sodiumsulfate, filtrated and concentrated to give 1.2 g (crude) of the title compound mixture.

LC-MS (Method 2): Rₜ=0.95/1.00 min; MS (ESIpos): m/z=382/398 [M+H]⁺

### Intermediate 147

### 8-cyclopropyl-N-[(4-methoxy-1H-benzimidazol-2-yl)methyl]-2-(methylsulfanyl)pyrazolo[1,5-a][1,3,5]triazin-4-amine

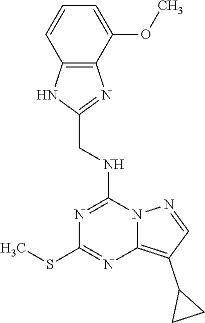 [SMILES: COc1cccc2[nH]c(CNc3nc(SC)nc4c(C5CC5)cnn34)nc12]

To dichloromethane (23 mL) was added 1-(4-methoxy-1H-benzimidazol-2-yl)methanamine hydrogen chloride (1/2) (600 mg, 2.40 mmol; CAS-RN:[175530-52-6]), N,N-diisopropylethylamine (1.3 mL, 7.2 mmol; CAS-RN:[7087-68-5]) and 4-chloro-8-cyclopropyl-2-(methylsulfanyl)pyrazolo[1,5-a][1,3,5]triazine (Intermediate 55, 577 mg, 2.40 mmol). The reaction mixture was stirred over night at rt. The mixture was concentrated under reduced pressure and treated with water. The precipitate was filtered off, washed with water and dried at 60° C. under reduced pressure to give 866 mg (92% yield) of the title compound.

LC-MS (Method 2): Rₜ=1.19 min; MS (ESIpos): m/z=382 [M+H]⁺

¹H-NMR (400 MHz, DMSO-d6) δ[ppm]: 0.820 (1.09), 0.825 (2.20), 0.832 (2.11), 0.837 (2.58), 0.843 (1.53), 0.850 (1.06), 0.861 (1.24), 0.867 (1.89), 0.872 (1.16), 0.881 (1.22), 0.888 (1.96), 0.904 (0.47), 0.906 (0.48), 1.855 (0.49), 1.858 (0.43), 1.862 (0.50), 1.871 (0.91), 1.875 (0.74), 1.884 (0.54), 1.888 (0.47), 1.892 (0.40), 1.896 (0.42), 2.413 (16.00), 2.518 (1.64), 2.522 (1.02), 3.882 (9.37), 3.894 (7.02), 4.855 (1.38), 4.869 (2.26), 4.881 (1.13), 6.632 (0.93), 6.634 (1.01), 6.652 (1.08), 6.654 (1.06), 6.731 (0.84), 6.749 (0.95), 6.979 (0.78), 6.981 (0.90), 6.999 (1.53), 7.001 (1.39), 7.027 (0.73), 7.036 (1.37), 7.047 (1.44), 7.055 (1.65), 7.067 (0.96), 7.075 (0.65), 7.121 (1.18), 7.142 (0.72), 7.913 (2.82), 7.931 (3.56), 9.119 (0.42), 9.224 (0.53), 12.247 (0.96), 12.550 (0.79).

### Intermediate 148

### 8-cyclopropyl-2-(methanesulfonyl)-N-[(4-methoxy-1H-benzimidazol-2-yl)methyl]pyrazolo[1,5-a][1,3,5]triazin-4-amine

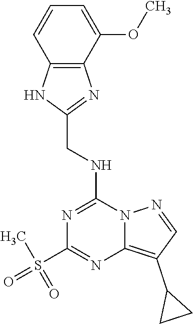 [SMILES: COc1cccc2[nH]c(CNc3nc(S(C)(=O)=O)nc4c(C5CC5)cnn34)nc12]

8-cyclopropyl-N-[(4-methoxy-1H-benzimidazol-2-yl)methyl]-2-(methylsulfanyl)-8,8a-dihydropyrazolo[1,5-a][1,3,5]triazin-4-amine (Intermediate 147, 860 mg, 2.24 mmol) was suspended in dichloromethane (25 mL). To the ice-bath cooled suspension was added in portions 3-chlorobenzene-1-carboperoxoic acid (1.51 g, 77% purity, 6.73 mmol; CAS-RN:[937-14-4]). The reaction mixture was stirred for 4 h at rt. Aqueous saturated sodium bicarbonate solution was added and the organic layer was separated. The aqueous layer was extracted with dichloromethane. The combined organic layer was dried over sodiumsulfate, filtrated and concentrated to give 987 mg (98% yield) of the title compound.

LC-MS (Method 2): Rₜ=0.95 min; MS (ESIpos): m/z=414 [M+H]⁺

¹H-NMR (400 MHz, DMSO-d6) δ[ppm]: 0.849 (0.80), 0.861 (2.11), 0.866 (2.16), 0.874 (2.31), 0.878 (2.06), 0.888 (0.85), 0.953 (1.23), 0.961 (2.32), 0.967 (1.84), 0.973 (1.40), 0.979 (1.20), 0.982 (2.34), 0.988 (1.73), 0.999 (0.68), 1.973 (0.69), 1.981 (0.75), 1.994 (1.21), 2.007 (0.63), 2.015 (0.59), 2.518 (2.14), 2.522 (1.36), 3.247 (15.47), 3.264 (0.68), 3.847 (0.48), 3.885 (16.00), 4.979 (1.08), 4.995 (1.56), 5.013 (0.98), 5.759 (2.83), 6.642 (0.68), 6.661 (0.74), 6.735 (0.61), 6.754 (0.68), 6.978 (0.57), 6.997 (0.92), 7.037 (0.47), 7.044 (0.78), 7.056 (0.94), 7.064 (1.00), 7.076 (0.57), 7.084 (0.42), 7.134 (0.80), 7.155 (0.51), 8.180 (1.57), 8.199 (1.81), 9.863 (0.42), 9.956 (0.44), 12.263 (0.77), 12.556 (0.71).

### Intermediate 149

### 8-bromo-N-[(5-methoxy-1H-benzimidazol-2-yl)methyl]-2-(methylsulfanyl)pyrazolo[1,5-a][1,3,5]triazin-4-amine hydrogen chloride (1/1)

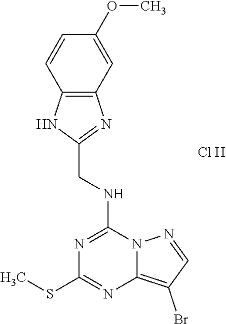 [SMILES: COc1ccc2[nH]c(CNc3nc(SC)nc4c(Br)cnn34)nc2c1 ; "Cl H"]

To dichloromethane (20 mL) was added 1-(5-methoxy-1H-benzimidazol-2-yl)methanamine-hydrogen chloride (1/2) (1.00 g, 4.00 mmol; CAS-RN:[175530-52-6]), N,N-diisopropylethylamine (1.4 mL, 8.0 mmol; CAS-RN:[7087-68-5]) and 8-bromo-4-chloro-2-(methylsulfanyl)pyrazolo[1,5-a][1,3,5]triazine (Intermediate 7, 1.12 g, 4.00 mmol). The reaction mixture was stirred 1 h at rt. The mixture was concentrated under reduced pressure and treated with water. The precipitate was filtered off, washed with water and ethanol and dried at 60° C. under reduced pressure to give 1.48 g (66% yield) of the title compound.

LC-MS (Method 2): Rₜ=1.10 min; MS (ESIpos): m/z=420 [M+H]⁺

### Intermediate 150

### 8-bromo-N-[(5-methoxy-1H-benzimidazol-2-yl)methyl]-2-methylsulfinyl-pyrazolo[1,5-a][1,3,5]triazin-4-amine

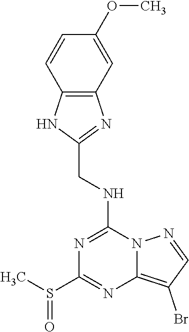 [SMILES: COc1ccc2[nH]c(CNc3nc(S(C)=O)nc4c(Br)cnn34)nc2c1]

8-bromo-N-[(5-methoxy-1H-benzimidazol-2-yl)methyl]-2-(methylsulfanyl)pyrazolo[1,5-a][1,3,5]triazin-4-amine hydrogen chloride (1/1) (Intermediate 149, 1.00 g, 82% purity, 1.80 mmol) was suspended in dichloromethane (25 mL). To the ice-bath cooled suspension was added in portions 3-chlorobenzene-1-carboperoxoic acid (1.21 g, 77% purity, 5.39 mmol; CAS-RN:[937-14-4]). The reaction mixture was stirred for 3 h at rt. Aqueous saturated sodium bicarbonate solution was added and the organic layer was separated. The aqueous layer was extracted with dichloromethane. The combined organic layers were dried over sodiumsulfate, filtrated and concentrated to give 1.1 g (crude) of the title compound.

LC-MS (Method 2): Rₜ=0.73 min; MS (ESIpos): m/z=436 [M+H]⁺

### Intermediate 151

### tert-butyl (1S,5R)-3-(4-{[(1H-benzimidazol-2-yl)methyl]amino}-8-bromopyrazolo[1,5-a][1,3,5]triazin-2-yl)-3,6-diazabicyclo[3.2.0]heptane-6-carboxylate

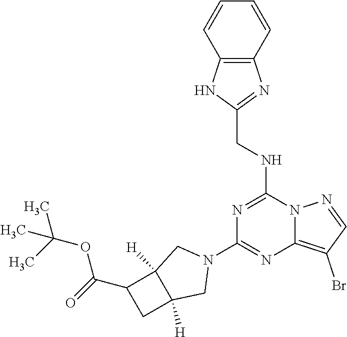 [SMILES: CC(C)(C)OC(=O)C1C[C@@H]2CN(c3nc(NCc4nc5ccccc5[nH]4)n4ncc(Br)c4n3)C[C@H]12]

N-[(1H-benzimidazol-2-yl)methyl]-8-bromo-2-(methanesulfonyl)pyrazolo[1,5-a][1,3,5]triazin-4-amine (Intermediate 9, 200 mg, 45% purity, 213 μmol) and tert-butyl (1S,5R)-3,6-diazabicyclo[3.2.0]heptane-6-carboxylate (52.8 mg, 266 μmol; CAS-RN:[370882-66-9]) were provided in acetonitrile (3 mL), N,N-diisopropylethylamine (110 μL, 640 μmol; CAS-RN:[7087-68-5]) was added and the mixture was stirred for 18 h at 70° C. Water was added and the mixture was extracted with ethylacetate. The combined organic layers were concentrated under reduced pressure. The residue was purified by flash chromatography (silica gel, dichloromethane/EtOH gradient) to give 111 mg (81% yield) of the title compound.

LC-MS (Method 2): Rₜ=1.21 min; MS (ESIpos): m/z=540 [M+H]⁺

### Intermediate 152

### tert-butyl [1-(4-{[(1H-benzimidazol-2-yl)methyl]amino}-8-bromopyrazolo[1,5-a][1,3,5]triazin-2-yl)piperidin-4-yl]carbamate

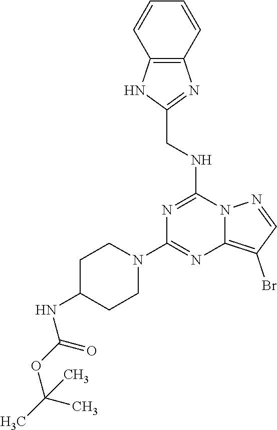 [SMILES: CC(C)(C)OC(=O)NC1CCN(c2nc(NCc3nc4ccccc4[nH]3)n3ncc(Br)c3n2)CC1]

N-[(1H-benzimidazol-2-yl)methyl]-8-bromo-2-(methanesulfonyl)pyrazolo[1,5-a][1,3,5]triazin-4-amine (Intermediate 9, 150 mg, 89% purity, 316 μmol) and tert-butyl piperidin-4-ylcarbamate (82.3 mg, 411 μmol; CAS-RN:[73874-95-0]) were provided in acetonitrile (2 mL), N,N-diisopropylethylamine (170 μL, 950 μmol; CAS-RN:[7087-68-5]) was added and the mixture was stirred for 5 h at 70° C. Water was added and the mixture was extracted with ethylacetate. The combined organic layers were concentrated under reduced pressure. The residue was purified by flash chromatography (silica gel, dichloromethane/EtOH gradient) to give 167 mg (93% yield) of the title compound.

LC-MS (Method 2): Rₜ=1.23 min; MS (ESIpos): m/z=542 [M+H]⁺

¹H-NMR (400 MHz, DMSO-d6) δ[ppm]: 1.038 (0.70), 1.055 (1.44), 1.073 (0.87), 1.372 (16.00), 2.524 (0.58), 2.898 (0.48), 4.850 (0.99), 4.864 (0.99), 7.108 (0.60), 7.113 (0.55), 7.117 (0.68), 7.124 (1.00), 7.131 (0.76), 7.136 (0.57), 7.141 (0.64), 7.393 (0.56), 7.410 (0.49), 7.415 (0.44), 7.523 (0.50), 7.528 (0.50), 7.545 (0.48), 8.010 (3.39), 9.057 (0.55), 12.267 (0.79).

### Intermediate 153

### 2-[[2-(chloromethyl)-5-methoxy-benzimidazol-1-yl]methoxy]ethyl-trimethyl-silane; 2-[[2-(chloromethyl)-6-methoxy-benzimidazol-1-yl]methoxy]ethyl-trimethyl-silane

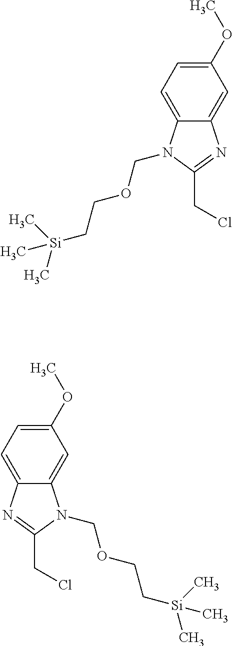 [SMILES: COc1ccc2c(c1)nc(CCl)n2COCC[Si](C)(C)C ; COc1ccc2nc(CCl)n(COCC[Si](C)(C)C)c2c1]

2-(chloromethyl)-5-methoxy-1H-benzimidazole (500 mg, 2.54 mmol; CAS-RN:[14625-40-2]) was dissolved in tetrahydrofurane (9.4 mL), [2-(chloromethoxy)ethyl](trimethyl)silane (490 μL, 2.8 mmol; CAS-RN:[76513-69-4]) and N,N-diisopropylethylamine (810 μL, 4.6 mmol; CAS-RN:[7087-68-5]) were added and the mixture was stirred over night at rt. The reaction mixture was concentrated under reduced pressure. The resulting residue was purified by flash chromatography (silica gel, dichloromethane/ethylacetate gradient) to give 497 mg (60% yield) of the title compound mixture.

LC-MS (Method 1): Rₜ=1.36 min; MS (ESIpos): m/z=327 [M+H]⁺

¹H-NMR (400 MHz, DMSO-d6) δ[ppm]: −0.079 (0.60), −0.071 (16.00), −0.067 (15.92), −0.063 (0.89), −0.059 (0.63), −0.046 (0.40), 0.000 (0.90), 0.839 (0.52), 0.851 (0.56), 0.859 (0.61), 0.870 (0.65), 0.879 (0.57), 0.890 (0.54), 3.520 (0.55), 3.539 (0.47), 3.541 (0.59), 3.548 (0.60), 3.561 (0.57), 3.569 (0.64), 3.589 (0.55), 3.806 (4.25), 3.831 (4.57), 5.038 (2.24), 5.049 (2.06), 5.678 (1.79), 5.691 (1.84), 6.883 (0.40), 6.890 (0.45), 6.905 (0.45), 6.912 (0.47), 6.964 (0.42), 6.987 (0.45), 7.187 (0.58), 7.192 (0.55), 7.233 (0.56), 7.239 (0.54), 7.539 (0.69), 7.562 (1.21), 7.584 (0.53).

### Intermediate 154

### N-[[5-methoxy-1-(2-trimethylsilylethoxymethyl)benzimidazol-2-yl]methyl]-2-morpholino-8-(trifluoromethyl)pyrazolo[1,5-a][1,3,5]triazin-4-amine; N-[[6-methoxy-1-(2-trimethylsilylethoxymethyl)benzimidazol-2-yl]methyl]-2-morpholino-8-(trifluoromethyl)pyrazolo[1,5-a][1,3,5]triazin-4-amine

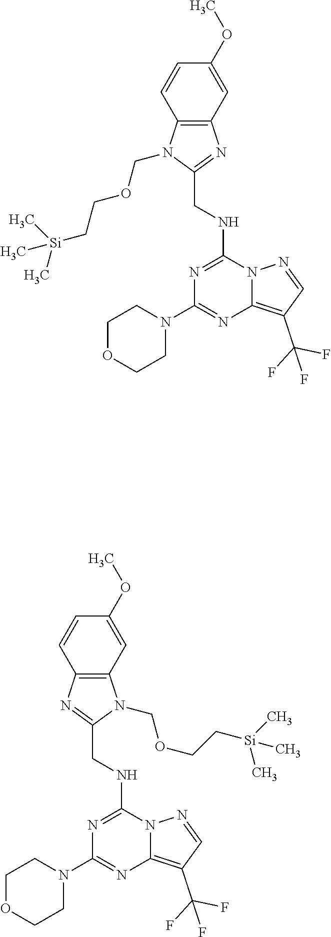 [SMILES: COc1ccc2c(c1)nc(CNc1nc(N3CCOCC3)nc3c(C(F)(F)F)cnn13)n2COCC[Si](C)(C)C ; COc1ccc2nc(CNc3nc(N4CCOCC4)nc4c(C(F)(F)F)cnn34)n(COCC[Si](C)(C)C)c2c1]

To a solution of 2-(morpholin-4-yl)-8-(trifluoromethyl)pyrazolo[1,5-a][1,3,5]triazin-4-amine (Intermediate 131, 100 mg, 347 μmol) in N,N-dimethylformamide (10.0 mL) was added sodium hydride (16.7 mg, 60% purity, 416 μmol; CAS-RN:[7646-69-7]) at rt. After stirring at 60° C. for 15 min, 2-[[2-(chloromethyl)-5-methoxy-benzimidazol-1-yl]methoxy]ethyl-trimethyl-silane;2-[[2-(chloromethyl)-6-methoxy-benzimidazol-1-yl]methoxy]ethyl-trimethyl-silane (Intermediate 153, 125 mg, 382 μmol) was added in one portion. The reaction mixture was stirred at rt for 16 h. The reaction mixture was concentrated under reduced pressure to give a residue. The residue was purified by flash chromatography (silica gel, dichloromethane/EtOAc gradient) to give 162 mg (81% yield) of the title compound mixture.

LC-MS (Method 1): Rₜ=1.46 min; MS (ESIpos): m/z=579 [M+H]⁺

¹H-NMR (400 MHz, DMSO-d6) δ[ppm]: −0.150 (0.48), −0.008 (4.33), 0.039 (1.34), 0.054 (0.77), 0.058 (1.49), 0.060 (1.24), 0.146 (0.47), 0.686 (2.60), 0.706 (3.67), 0.727 (2.70), 1.304 (0.58), 1.321 (1.19), 1.339 (0.59), 1.380 (0.46), 2.137 (2.09), 2.668 (5.46), 2.672 (3.89), 3.515 (1.91), 3.538 (3.37), 3.557 (3.59), 3.579 (2.52), 3.609 (1.28), 3.620 (1.31), 3.640 (1.11), 3.788 (4.87), 3.796 (5.76), 3.862 (1.00), 3.875 (0.96), 3.885 (0.57), 3.894 (0.50), 3.906 (14.35), 3.935 (16.00), 3.943 (1.61), 3.958 (0.49), 4.167 (0.43), 4.185 (0.44), 5.075 (2.27), 5.083 (2.67), 5.088 (2.79), 5.096 (2.17), 5.756 (4.66), 5.767 (5.19), 6.947 (1.48), 6.953 (1.46), 6.969 (1.43), 6.975 (1.65), 7.008 (1.22), 7.014 (1.32), 7.030 (1.29), 7.036 (1.40), 7.286 (2.20), 7.292 (2.16), 7.327 (2.13), 7.332 (2.04), 7.605 (2.62), 7.626 (2.58), 7.632 (2.08), 7.655 (1.87), 8.324 (0.92), 8.375 (4.11), 8.379 (3.78), 9.329 (0.52), 9.343 (1.16), 9.356 (0.94), 9.369 (1.06), 9.383 (0.49).

### Intermediate 155

### tert-butyl [3-(4-{[(1H-benzimidazol-2-yl)methyl]amino}-8-bromopyrazolo[1,5-a][1,3,5]triazin-2-yl)-3-azabicyclo[3.1.0]hexan-1-yl]carbamate

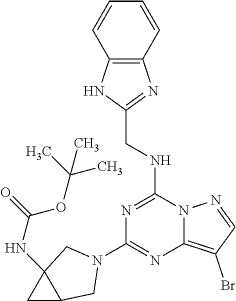 [SMILES: CC(C)(C)OC(=O)NC12CC1CN(c1nc(NCc3nc4ccccc4[nH]3)n3ncc(Br)c3n1)C2]

N-[(1H-benzimidazol-2-yl)methyl]-8-bromo-2-(methanesulfonyl)pyrazolo[1,5-a][1,3,5]triazin-4-amine (Intermediate 9; 200 mg, 45% purity, 213 μmol) and tert-butyl 3-azabicyclo[3.1.0]hexan-1-ylcarbamate (127 mg, 639 μmol; CAS-RN:[204991-14-0]) were provided in acetonitrile (3.0 mL), N,N-diisopropylethylamine (110 μL, 640 μmol; CAS-RN:[7087-68-5]) was added and the mixture was stirred for 16 h at 70° C. Water was added and the mixture was extracted with ethylacetate. The combined organic layers were concentrated under reduced pressure. The residue was purified by flash chromatography (silica gel, dichloromethane/EtOH gradient) to give 139 mg of the title compound.

LC-MS (Method 2): Rₜ=1.20 min; MS (ESIpos): m/z=540 [M+H]⁺

### Intermediate 156

### tert-butyl [(7R)-5-(4-{[(1H-benzimidazol-2-yl)methyl]amino}-8-bromopyrazolo[1,5-a][1,3,5]triazin-2-yl)-5-azaspiro[2.4]heptan-7-yl]carbamate

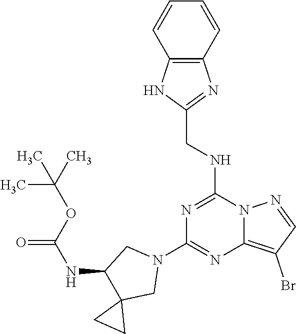 [SMILES: CC(C)(C)OC(=O)N[C@H]1CN(c2nc(NCc3nc4ccccc4[nH]3)n3ncc(Br)c3n2)CC12CC2]

N-[(1H-benzimidazol-2-yl)methyl]-8-bromo-2-(methanesulfonyl)pyrazolo[1,5-a][1,3,5]triazin-4-amine (Intermediate 9; 200 mg, 45% purity, 213 μmol) and tert-butyl (7R)-5-azaspiro[2.4]heptan-7-ylcarbamate (136 mg, 639 μmol; CAS-RN:[127199-44-4]) were provided in acetonitrile (3.0 mL), N,N-diisopropylethylamine (110 μL, 640 μmol; CAS-RN:[7087-68-5]) was added and the mixture was stirred for 23 h at 70° C. Water was added and the mixture was extracted with ethylacetate. The combined organic layers were concentrated under reduced pressure. The residue was purified by flash chromatography (silica gel, dichloromethane/EtOH gradient) to give 151 mg (91% yield) of the title compound.

LC-MS (Method 2): Rₜ=1.27 min; MS (ESIpos): m/z=554 [M+H]⁺

### Intermediate 157

### 2-(chloromethyl)-6,7-difluoro-1H-benzimidazole

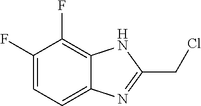 [SMILES: Fc1ccc2nc(CCl)[nH]c2c1F]

A solution of 3,4-difluorobenzene-1,2-diamine (2.50 g, 17.3 mmol; CAS-RN:[153505-39-6]) and chloroacetic acid (2.46 g, 26.0 mmol) in hydrochloric acid (5 M) was stirred at 100° C. for 16 h. The reaction mixture was cooled to rt. Saturated sodium carbonate solution was added to the reaction mixture to adjust pH=7-8. The mixture was filtered and the filter cake was washed with water and dried to give 2.8 g (80% yield) of the title compound as a yellow solid.

LC-MS (Method D): Rₜ=0.658 min; MS (ESIpos): m/z=203.1 [M+H]⁺

### Intermediate 158

### 2-(chloromethyl)-6,7-difluoro-1-{[2-(trimethylsilyl)ethoxy]methyl}-1H-benzimidazole

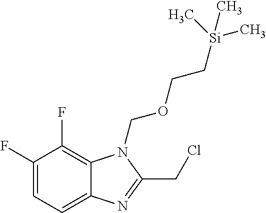 [SMILES: C[Si](C)(C)CCOCn1c(CCl)nc2ccc(F)c(F)c21]

To a solution of 2-(chloromethyl)-6,7-difluoro-1H-benzimidazole (Intermediate 157, 2.50 g, 12.3 mmol) and [2-(chloromethoxy)ethyl](trimethyl)silane (2.47 g, 14.8 mmol; CAS-RN:[76513-69-4]) in tetrahydrofuran (15.0 mL) was added N,N-diisopropylethylamine (4.3 mL, 25 mmol; CAS-RN:[7087-68-5]) at rt. The reaction mixture was stirred at rt for 16 h. The reaction mixture was diluted with water and extracted with ethyl acetate. The organic phase was washed with brine, dried over anhydrous sodium sulfate and filtered. The filtrate was concentrated and purified by column chromatography on silica gel (200-300 mesh, petroleum ether: ethyl acetate=50:1) to give 3.0 g (73% yield) of the title compound (50% impure with [2-(chloromethyl)-4,5-difluoro-benzimidazol-1-yl]methoxymethyl-trimethyl-silane) as a yellow oil.

LC-MS (Method D): Rₜ=0.996 min; MS (ESIpos): m/z=333.1 [M+H]⁺

### Intermediate 159

### 8-cyclopentyl-N-[(4,5-difluoro-1-{[2-(trimethylsilyl)ethoxy]methyl}-1H-benzimidazol-2-yl)methyl]-2-(morpholin-4-yl)pyrazolo[1,5-a][1,3,5]triazin-4-amine-8-cyclopentyl-N-[(6,7-difluoro-1-{[2-(trimethylsilyl)ethoxy]methyl}-1H-benzimidazol-2-yl)methyl]-2-(morpholin-4-yl)pyrazolo[1,5-a][1,3,5]triazin-4-amine (1/1)

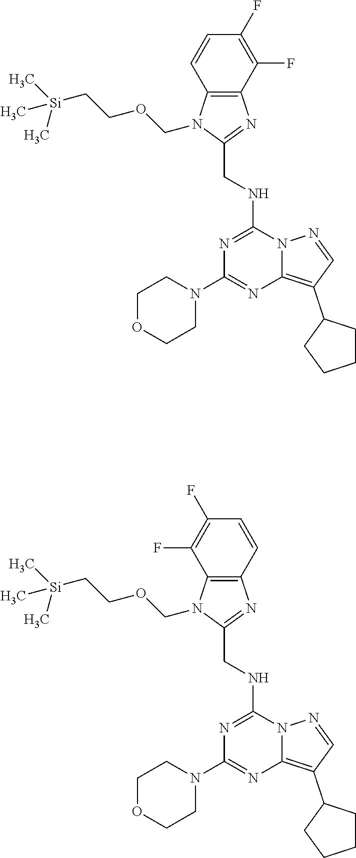 [SMILES: C[Si](C)(C)CCOCn1c(CNc2nc(N3CCOCC3)nc3c(C4CCCC4)cnn23)nc2c(F)c(F)ccc21 ; C[Si](C)(C)CCOCn1c(CNc2nc(N3CCOCC3)nc3c(C4CCCC4)cnn23)nc2ccc(F)c(F)c21]

To a solution of 8-cyclopentyl-2-(morpholin-4-yl)pyrazolo[1,5-a][1,3,5]triazin-4-amine (Intermediate 138, 100.0 mg, 347 μmol) in N,N-dimethylformamide (2.0 mL) was added sodium hydride (16.6 mg, 60% purity, 416 μmol; CAS-RN:[7646-69-7]) at rt. After stirring at 60° C. for 15 min, 2-(chloromethyl)-6,7-difluoro-1-{[2-(trimethylsilyl)ethoxy]methyl}-1H-benzimidazole (50% impure with [2-(chloromethyl)-4,5-difluoro-benzimidazol-1-yl]methoxymethyl-trimethyl-silane) (Intermediate 158, 139 mg, 416 μmol) was added in one portion. The reaction mixture was stirred at rt for 16 h. The reaction mixture was concentrated under reduced pressure to give a residue. The residue was diluted with water and extracted with ethyl acetate. The organic phase was washed with brine, dried over anhydrous sodium sulfate and filtered. The filtrate was concentrated to give a residue. The residue was purified by preparative TLC (petroleum ether: ethyl acetate=3:1) to give 40.0 mg (85% purity, 17% yield) of the title compound as yellow solid.

LC-MS (Method D): Rₜ=1.046 min; MS (ESIpos): m/z=585.4 [M+H]⁺

### Intermediate 160

### ethyl [(1H-pyrazol-5-yl)carbamothioyl]carbamate

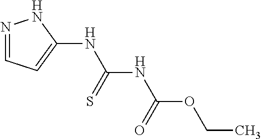

To a stirred suspension of 1H-pyrazol-5-amine (150 g, 1.81 mol; CAS-RN:[916420-28-5]) in a mixed solvent of ethyl acetate (600 mL) and toluene (2.5 l) was added dropwise a solution of ethyl carbonisothiocyanatidate (237 g, 1.81 mol; CAS-RN:[16182-04-0]) in toluene (500 mL) at 5° C. under nitrogen atmosphere. After stirring for 24 h at rt, the resulting precipitate was collected by filtration, and washed with toluene to give 270 g (70% yield) of the title compound as a yellow solid.

LC-MS (Method D): Rₜ=0.633 min; MS (ESIpos): m/z=215.2 [M+H]⁺

### Intermediate 161

### 2-sulfanylidene-2,3-dihydropyrazolo[1,5-a][1,3,5]triazin-4(1H)-one

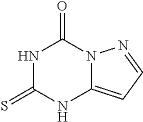 [SMILES: O=c1[nH]c(=S)[nH]c2ccnn12]

A solution of ethyl (1H-pyrazol-5-ylcarbamothioyl)carbamate (Intermediate 160, 270 g, 1.26 mol) in aqueous sodium hydroxide (3.1 l, 2.0 M, 6.1 mol; CAS-RN:[1310-73-2]) was stirred for 1.5 h at rt. To the stirred reaction mixture was added 2 M sulfuric acid. The resulting precipitates were collected by filtration, washed with water and dried under reduced pressure to give 156 g (90% purity, 66% yield) of the title compound as a yellow solid.

LC-MS (Method D): Rₜ=0.181 min; MS (ESIpos): m/z=169.1 [M+H]⁺

### Intermediate 162

### 2-(methylsulfanyl)pyrazolo[1,5-a][1,3,5]triazin-4(3H)-one

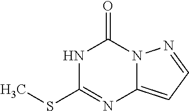

To a stirred solution of 2-sulfanylidene-2,3-dihydropyrazolo[1,5-a][1,3,5]triazin-4(1H)-one (Intermediate 161, 78.0 g, 90% purity, 417 mmol) in ethanol (2.0 L) and sodium hydroxide (500 mL, 1.7 M, 870 mmol) was added methyl iodide (26 mL, 420 mmol; CAS-RN:[74-88-4]) at ambient temperature. The resulting precipitate was collected by filtration, and then dissolved in water. To the resulting solution was added 2 M sulfuric acid. The resulting precipitate was collected by filtration, washed with water, and then dried under reduced pressure to give 106.0 g (crude) of the title compound as a white solid.

LC-MS (Method D): Rₜ=0.623 min; MS (ESIpos): m/z=183.1 [M+H]⁺

### Intermediate 163

### 1-[4-{bis[(4-methoxyphenyl)methyl]amino}-2-(morpholin-4-yl)pyrazolo[1,5-a][1,3,5]triazin-8-yl]cyclobutan-1-01

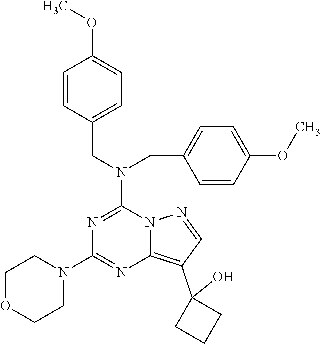 [SMILES: COc1ccc(CN(Cc2ccc(OC)cc2)c2nc(N3CCOCC3)nc3c(C4(O)CCC4)cnn23)cc1]

To a solution of 8-iodo-N,N-bis[(4-methoxyphenyl)methyl]-2-(morpholin-4-yl)pyrazolo[1,5-a][1,3,5]triazin-4-amine (Intermediate 128, 2.00 g, 3.41 mmol) in tetrahydrofuran (40 mL) was added isopropylmagnesium chloride (3.4 mL, 2.0 M in tetrahydrofuran, 6.8 mmol; CAS-RN:[1068-55-9]) dropwise at 25° C. After stirring for 10 min, cyclobutanone (478 mg, 6.82 mmol; CAS-RN:[1191-95-3]) was added to the above mixture and stirred at 25° C. for 30 min. The mixture was quenched with water, diluted with water and extracted with ethyl acetate. The combined organic layers were washed with water and brine, dried over anhydrous sodium sulfate and concentrated under reduced pressure to give 1.95 g (73% purity, 79% yield) of the title compound as a yellow oil.

LC-MS (Method C): Rₜ=0.94 min; MS (ESIpos): m/z=531 [M+H]⁺

### Intermediate 164

### 8-cyclobutyl-N-[(4-methoxyphenyl)methyl]-2-(morpholin-4-yl)pyrazolo[1,5-a][1,3,5]triazin-4-amine

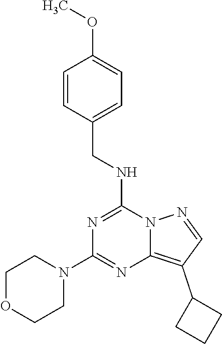 [SMILES: COc1ccc(CNc2nc(N3CCOCC3)nc3c(C4CCC4)cnn23)cc1]

A solution of 1-[4-{bis[(4-methoxyphenyl)methyl]amino}-2-(morpholin-4-yl)pyrazolo[1,5-a][1,3,5]triazin-8-yl]cyclobutan-1-ol (Intermediate 163, 1.90 g, 3.58 mmol) in trifluoroacetic acid was added triethylsilane (4.0 mL) at 25° C. The mixture was stirred at 80° C. for 3 h. The solution was concentrated to give the crude product. The crude was purified by silica gel column chromatography (100-200 mesh, petroleum ether: ethyl acetate=10: 1-3:1) to give 1.30 g (98% purity, 90% yield) of the title compound as a white solid.

LC-MS (Method C): Rₜ=0.94 min; MS (ESIpos): m/z=395 [M+H]⁺

### Intermediate 165

### 8-cyclobutyl-N-[(4-methoxyphenyl)methyl]-2-morpholino-N-[[1-(2-trimethylsilylethoxymethyl)benzimidazol-2-yl]methyl]pyrazolo[1,5-a][1,3,5]triazin-4-amine

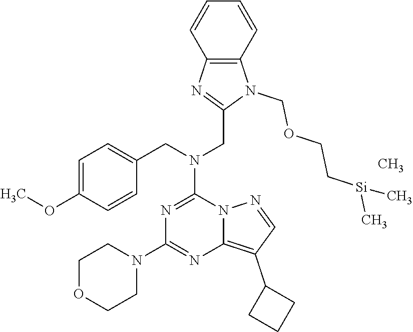

To a solution of 8-cyclobutyl-N-[(4-methoxyphenyl)methyl]-2-(morpholin-4-yl)pyrazolo[1,5-a][1,3,5]triazin-4-amine (Intermediate 164, 200 mg, 507 μmol) in N,N-dimethylformamide (4.0 mL) was added sodium hydride (30.4 mg, 60% purity, 760 μmol) at rt. After stirring at 60° C. for 20 min, 2-(chloromethyl)-1-{[2-(trimethylsilyl)ethoxy]methyl}-1H-benzimidazole (Intermediate 130, 181 mg, 608 μmol) was added in one portion. The reaction mixture was stirred at rt for 16 h. The reaction mixture was poured into water. The mixture was extracted with ethyl acetate. The organic phase was washed with brine, dried over anhydrous sodium sulfate and filtered. The filtrate was concentrated under reduced pressure to give 400 mg (67% purity, 81% yield) of the title compound as a yellow oil.

LC-MS (Method C): Rₜ=0.99 min; MS (ESIpos): m/z=655 [M+H]⁺

### Intermediate 166

### 8-cyclobutyl-N-[(4,5-difluoro-1-{[2-(trimethylsilyl)ethoxy]methyl}-1H-benzimidazol-2-yl)methyl]-N-[(4-methoxyphenyl)methyl]-2-(morpholin-4-yl)pyrazolo[1,5-a][1,3,5]triazin-4-amine

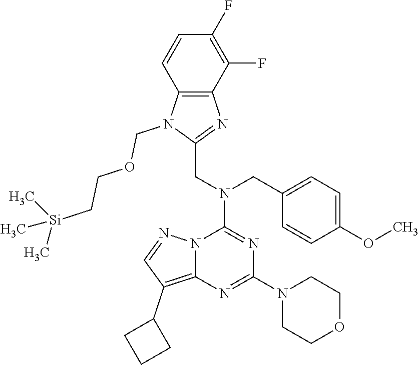

To a solution of 8-cyclobutyl-N-[(4-methoxyphenyl)methyl]-2-(morpholin-4-yl)pyrazolo[1,5-a][1,3,5]triazin-4-amine (Intermediate 164, 200 mg, 507 μmol) in N,N-dimethylformamide (4.0 mL) was added sodium hydride (30.4 mg, 60% purity, 760 μmol) at rt. After stirring at 60° C. for 20 min, 2-(chloromethyl)-6,7-difluoro-1-{[2-(trimethylsilyl)ethoxy]methyl}-1H-benzimidazole (50% impure with [2-(chloromethyl)-4,5-difluoro-benzimidazol-1-yl]methoxymethyl-trimethyl-silane) (Intermediate 158, 253 mg, 760 μmol) was added in one portion. The reaction mixture was stirred at rt for 16 h. The reaction mixture was poured into water. The mixture was extracted with ethyl acetate. The organic phase was washed with brine, dried over anhydrous sodium sulfate and filtered. The filtrate was concentrated under reduced pressure to give 410 mg (84% purity, 98% yield) of the title compound as a yellow oil.

LC-MS (Method C): Rₜ=1.23 min; MS (ESIpos): m/z=692 [M+H]⁺

### Intermediate 167

### 4-{bis[(4-methoxyphenyl)methyl]amino}-2-(morpholin-4-yl)pyrazolo[1,5-a][1,3,5]triazine-8-carbaldehyde

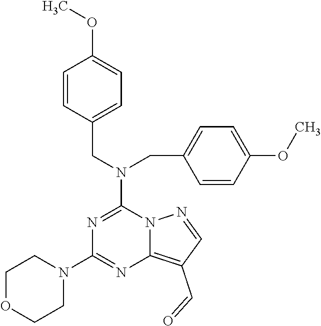 [SMILES: COc1ccc(CN(Cc2ccc(OC)cc2)c2nc(N3CCOCC3)nc3c(C=O)cnn23)cc1]

To a solution of 8-iodo-N,N-bis[(4-methoxyphenyl)methyl]-2-(morpholin-4-yl)pyrazolo[1,5-a][1,3,5]triazin-4-amine (Intermediate 128, 2.00 g, 3.41 mmol) in tetrahydrofuran was added isopropylmagnesium chloride (3.4 mL, 2.0 M in tetrahydrofuran, 6.8 mmol) at 0° C. After stirring at 25° C. for 1 h, N,N-dimethylformamide (2.0 mL) was added dropwised to the above mixture, and stirred at 25° C. for another 1 h. The solution was diluted with water and extracted with ethyl acetate. The combined organic layers were washed with water and brine, dried over anhydrous sodium sulfate and concentrated to give the crude product. The crude was purified by silica gel column chromatography (100-200 mesh, petroleum ether: ethyl acetate=5:1, then 2:1) to give 1.20 g (89% purity, 64% yield) of the title compound as a yellow solid.

LC-MS (Method C): Rₜ=1.01 min; MS (ESIpos): m/z=490 [M+H]⁺

### Intermediate 168

### 1-[4-{bis[(4-methoxyphenyl)methyl]amino}-2-(morpholin-4-yl)pyrazolo[1,5-a][1,3,5]triazin-8-yl]-2,2,2-trifluoroethan-1-ol

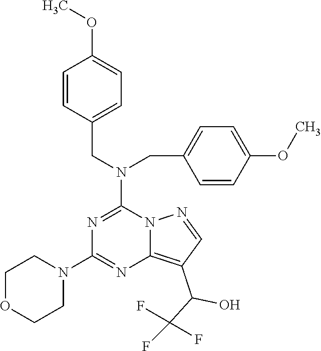 [SMILES: COc1ccc(CN(Cc2ccc(OC)cc2)c2nc(N3CCOCC3)nc3c(C(O)C(F)(F)F)cnn23)cc1]

To a solution of 4-{bis[(4-methoxyphenyl)methyl]amino}-2-(morpholin-4-yl)pyrazolo[1,5-a][1,3,5]triazine-8-carbaldehyde (Intermediate 167, 1.00 g, 2.05 mmol) and cesium fluoride (622 mg, 4.09 mmol; CAS-RN:[13400-13-0]) in tetrahydrofuran (40 mL) was added dropwise trimethyl(trifluoromethyl)silane (582 mg, 4.09 mmol; CAS-RN:[81290-20-2]) at 0° C. The mixture was stirred at 25° C. for 3 h. The solution was diluted with water and extracted with ethyl acetate. The combined organic layers were washed with water and brine, dried over anhydrous sodium sulfate and concentrated under reduced pressure to give 1.20 g (58% purity, 61% yield) of the title compound as a yellow oil.

LC-MS (Method C): Rₜ=0.98 min; MS (ESIpos): m/z=559 [M+H]⁺

### Intermediate 169

### N-[(4-methoxyphenyl)methyl]-2-(morpholin-4-yl)-8-(2,2,2-trifluoroethyl)pyrazolo[1,5-a][1,3,5]triazin-4-amine

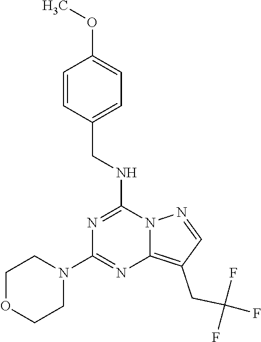 [SMILES: COc1ccc(CNc2nc(N3CCOCC3)nc3c(CC(F)(F)F)cnn23)cc1]

A solution of 1-[4-{bis[(4-methoxyphenyl)methyl]amino}-2-(morpholin-4-yl)pyrazolo[1,5-a][1,3,5]triazin-8-yl]-2,2,2-trifluoroethan-1-ol (Intermediate 168, 1.20 g, 58% purity, 1.25 mmol) in the mix solvent trifluoroacetic acid (10 mL) and triethylsilane (5.0 mL) was stirred at 80° C. for 3 h. The mixture was concentrated and purified by silica gel column chromatography (100-200 mesh, petroleum ether: ethyl acetate=5:1, then 3:1) to give 500 mg (98% purity, 93% yield) of the title compound as a white solid.

LC-MS (Method C): Rₜ=0.89 min; MS (ESIpos): m/z=423 [M+H]⁺

### Intermediate 170

### N-[(4-methoxyphenyl)methyl]-2-(morpholin-4-yl)-8-(2,2,2-trifluoroethyl)-N-[(1-{[2-(trimethylsilyl)ethoxy]methyl}-1H-benzimidazol-2-yl)methyl]pyrazolo[1,5-a][1,3,5]triazin-4-amine

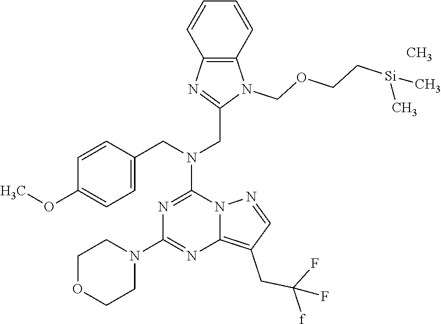

To a solution of sodium hydride (28.4 mg, 60% purity, 710 μmol; CAS-RN:[7646-69-7]) in N,N-dimethylformamide (3.0 mL) was added N-[(4-methoxyphenyl)methyl]-2-(morpholin-4-yl)-8-(2,2,2-trifluoroethyl)pyrazolo[1,5-a][1,3,5]triazin-4-amine (Intermediate 169, 150 mg, 355 μmol) at 25° C. After stirring at 60° C. for 20 min. 2-(chloromethyl)-1-{[2-(trimethylsilyl)ethoxy]methyl}-1H-benzimidazole (Intermediate 130, 158 mg, 533 μmol) was added to the above mixture at 25° C., and the mixture was stirred at 25° C. for 16 h. The reaction was quenched with water carefully. The solution was diluted with water and extracted with ethyl acetate. The combined organic layers were washed with water and brine, dried over anhydrous sodium sulfate and concentrated under reduced pressure to give 100 mg (80% purity, 33% yield) of the title compound as yellow oil.

LC-MS (Method C): Rₜ=0.99 min; MS (ESIpos): m/z=683 [M+H]⁺

### Intermediate 171

### tert-butyl N-[1-[4-(1H-benzimidazol-2-ylmethylamino)-8-bromo-pyrazolo[1,5-a][1,3,5]triazin-2-yl]-3-(trifluoromethyl)pyrrolidin-3-yl]carbamate

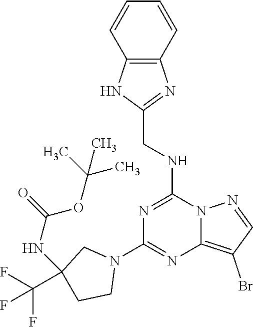 [SMILES: CC(C)(C)OC(=O)NC1(C(F)(F)F)CCN(c2nc(NCc3nc4ccccc4[nH]3)n3ncc(Br)c3n2)C1]

N-[(1H-benzimidazol-2-yl)methyl]-8-bromo-2-(methanesulfonyl)pyrazolo[1,5-a][1,3,5]triazin-4-amine (Intermediate 9, 150 mg, 75% purity, 266 μmol) and tert-butyl [(3R)-3-(trifluoromethyl)pyrrolidin-3-yl]carbamate (102 mg, 400 μmol; CAS-RN:[186203-13-4]) were provided in acetonitrile (3 mL), N,N-diisopropylethylamine (140 μL, 800 μmol; CAS-RN:[7087-68-5]) was added and the mixture was stirred for 12 h at 70° C. Water was added and the mixture was extracted with ethylacetate. The combined organic layers were concentrated under reduced pressure. The residue was purified by flash chromatography (silica gel, dichloromethane/EtOH gradient) to give 150 mg (88% yield) of the title compound.

LC-MS (Method 2): Rₜ=1.32 min; MS (ESIpos): m/z=596 [M+H]⁺

¹H-NMR (400 MHz, DMSO-d6) δ[ppm]: 1.039 (0.42), 1.056 (0.88), 1.073 (0.50), 1.234 (0.75), 1.370 (16.00), 1.391 (3.59), 1.395 (2.65), 2.268 (0.70), 2.325 (0.64), 2.330 (0.80), 2.334 (0.61), 2.526 (2.00), 2.543 (1.11), 2.571 (0.92), 2.667 (0.61), 2.672 (0.76), 2.676 (0.57), 3.409 (0.56), 3.426 (0.64), 3.555 (0.72), 3.870 (0.81), 3.903 (0.98), 4.094 (0.64), 4.127 (0.56), 4.888 (1.97), 4.902 (2.35), 5.760 (2.57), 7.101 (0.76), 7.115 (2.37), 7.119 (2.24), 7.124 (2.75), 7.131 (3.98), 7.137 (2.85), 7.142 (2.31), 7.147 (2.49), 7.161 (0.77), 7.397 (2.23), 7.403 (1.36), 7.413 (1.92), 7.418 (1.70), 7.529 (1.92), 7.550 (1.83), 7.597 (0.42), 7.681 (0.47), 8.041 (11.98), 9.082 (0.48), 9.135 (0.91), 12.251 (1.46).

### Intermediate 172

### tert-butyl (1R,5S)-3-(4-{[(1H-benzimidazol-2-yl)methyl]amino}-8-bromopyrazolo[1,5-a][1,3,5]triazin-2-yl)-3,6-diazabicyclo[3.2.0]heptane-6-carboxylate

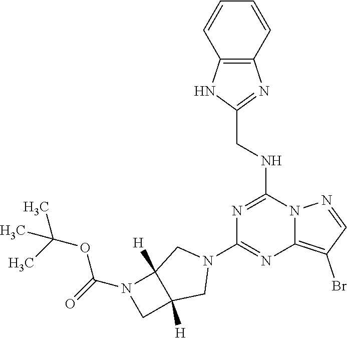 [SMILES: CC(C)(C)OC(=O)N1C[C@H]2CN(c3nc(NCc4nc5ccccc5[nH]4)n4ncc(Br)c4n3)C[C@H]21]

N-[(1H-benzimidazol-2-yl)methyl]-8-bromo-2-(methanesulfonyl)pyrazolo[1,5-a][1,3,5]triazin-4-amine (Intermediate 9, 200 mg, 45% purity, 213 μmol) and tert-butyl (1R,5S)-3,6-diazabicyclo[3.2.0]heptane-6-carboxylate (127 mg, 639 μmol; CAS-RN:[799279-81-5]) were provided in acetonitrile (3 mL), N,N-diisopropylethylamine (110 μL, 640 μmol; CAS-RN:[7087-68-5]) was added and the mixture was stirred for 18 h at 70° C. Water was added and the mixture was extracted with ethylacetate. The combined organic layers were concentrated under reduced pressure. The residue was purified by flash chromatography (silica gel, dichloromethane/EtOH gradient) to give 113 mg (88% yield) of the title compound.

LC-MS (Method 2): Rₜ=1.21 min; MS (ESIpos): m/z=540 [M+H]⁺

### Intermediate 173

### N-[(4,5-difluoro-1-{[2-(trimethylsilyl)ethoxy]methyl}-1H-benzimidazol-2-yl)methyl]-N-[(4-methoxyphenyl)methyl]-2-(morpholin-4-yl)-8-(2,2,2-trifluoroethyl)pyrazolo[1,5-a][1,3,5]triazin-4-amine

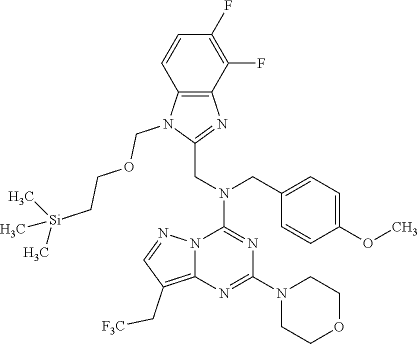

To a solution of sodium hydride (28.4 mg, 60% purity, 710 μmol; CAS-RN:[7646-69-7]) in N,N-dimethylformamide (4.0 mL) was added N-[(4-methoxyphenyl)methyl]-2-(morpholin-4-yl)-8-(2,2,2-trifluoroethyl)pyrazolo[1,5-a][1,3,5]triazin-4-amine (Intermediate 169, 150 mg, 355 μmol) at 25° C. After stirring at 60° C. for 20 min 2-(chloromethyl)-6,7-difluoro-1-{[2-(trimethylsilyl)ethoxy]methyl}-1H-benzimidazole (50% impure with [2-(chloromethyl)-4,5-difluoro-benzimidazol-1-yl]methoxymethyl-trimethyl-silane) (Intermediate 158, 177 mg, 533 μmol) was added to the above mixture at 25° C., and the mixture was stirred at 25° C. for 16 h. The reaction mixture was poured into water. The mixture was extracted with ethyl acetate. The organic phase was washed with brine, dried over anhydrous sodium sulfate and filtered. The filtrate was concentrated to give 250 mg (87% purity, 85% yield) of the title compound as a yellow oil.

LC-MS (Method C): Rₜ=1.21 min; MS (ESIpos): m/z=720 [M+H]⁺

### Intermediate 174

### tert-butyl (1R,4R)-5-(4-{[(1H-benzimidazol-2-yl)methyl]amino}-8-bromopyrazolo[1,5-a][1,3,5]triazin-2-yl)-2,5-diazabicyclo[2.2.1]heptane-2-carboxylate

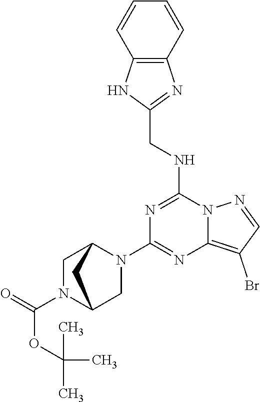 [SMILES: CC(C)(C)OC(=O)N1C[C@H]2C[C@@H]1CN2c1nc(NCc2nc3ccccc3[nH]2)n2ncc(Br)c2n1]

N-[(1H-benzimidazol-2-yl)methyl]-8-bromo-2-(methanesulfonyl)pyrazolo[1,5-a][1,3,5]triazin-4-amine (Intermediate 9, 150 mg, 75% purity, 266 μmol) and tert-butyl (1R,4R)-2,5-diazabicyclo[2.2.1]heptane-2-carboxylate (68.7 mg, 346 μmol; CAS-RN:[134003-84-2]) were provided in acetonitrile (2 mL), N,N-diisopropylethylamine (140 μL, 800 μmol; CAS-RN:[7087-68-5]) was added and the mixture was stirred for 23 h at 70° C. Water was added and the mixture was extracted with ethylacetate The combined organic layers were concentrated under reduced pressure. The residue was purified by flash chromatography (silica gel, dichloromethane/EtOH gradient) to give 147 mg (99% yield) of the title compound.

LC-MS (Method 2): Rₜ=1.21 min; MS (ESIpos): m/z=540 [M+H]⁺

¹H-NMR (400 MHz, DMSO-d6) δ[ppm]: 0.801 (0.72), 0.804 (0.65), 0.817 (0.74), 0.824 (0.86), 0.887 (1.58), 0.905 (3.06), 0.923 (1.65), 0.994 (0.90), 1.012 (0.81), 1.038 (2.49), 1.056 (5.53), 1.073 (2.87), 1.091 (0.49), 1.162 (0.71), 1.234 (1.00), 1.277 (8.13), 1.307 (16.00), 1.323 (7.12), 1.383 (13.67), 1.714 (0.48), 1.738 (0.83), 1.805 (2.80), 1.908 (0.41), 2.068 (4.27), 2.094 (0.63), 2.325 (1.09), 2.330 (1.50), 2.334 (1.08), 2.422 (0.98), 2.441 (1.11), 2.455 (1.47), 2.521 (6.11), 2.526 (4.04), 2.667 (1.46), 2.672 (2.11), 2.676 (1.71), 2.699 (0.77), 2.812 (0.77), 2.835 (0.58), 2.945 (2.29), 3.204 (0.67), 3.227 (1.17), 3.252 (0.58), 3.407 (1.93), 3.426 (1.39), 3.437 (1.47), 3.443 (1.44), 3.455 (1.52), 3.483 (0.55), 4.308 (0.73), 4.348 (1.57), 4.360 (1.15), 4.375 (2.23), 4.664 (1.22), 4.704 (1.23), 4.811 (1.71), 4.830 (2.14), 4.845 (2.07), 4.902 (0.78), 5.761 (2.41), 7.120 (3.76), 7.380 (2.31), 7.400 (2.09), 7.526 (2.25), 7.538 (2.00), 8.027 (6.36), 9.126 (1.51), 9.183 (0.74), 12.219 (2.64).

### Intermediate 175

### tert-butyl (1S,4S)-5-(4-{[(1H-benzimidazol-2-yl)methyl]amino}-8-bromopyrazolo[1,5-a][1,3,5]triazin-2-yl)-2,5-diazabicyclo[2.2.1]heptane-2-carboxylate

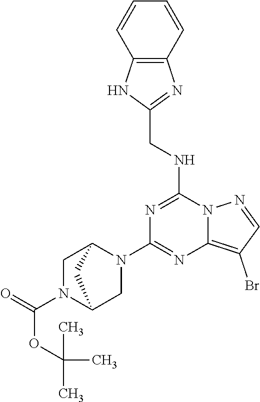 [SMILES: CC(C)(C)OC(=O)N1C[C@@H]2C[C@H]1CN2c1nc(NCc2nc3ccccc3[nH]2)n2ncc(Br)c2n1]

N-[(1H-benzimidazol-2-yl)methyl]-8-bromo-2-(methanesulfonyl)pyrazolo[1,5-a][1,3,5]triazin-4-amine (Intermediate 9, 150 mg, 75% purity, 266 μmol) and tert-butyl (1S,4S)-2,5-diazabicyclo[2.2.1]heptane-2-carboxylate (68.7 mg, 346 μmol; CAS-RN:[113451-59-5]) were provided in acetonitrile (2 mL), N,N-diisopropylethylamine (140 μL, 800 μmol; CAS-RN:[7087-68-5]) was added and the mixture was stirred for 23 h at 70° C. Water was added and the mixture was extracted with ethylacetate The combined organic layers were concentrated under reduced pressure. The residue was purified by flash chromatography (silica gel, dichloromethane/EtOH gradient) to give 148 mg (99% yield) of the title compound.

LC-MS (Method 2): Rₜ=1.21 min; MS (ESIpos): m/z=540 [M+H]⁺

¹H-NMR (400 MHz, DMSO-d6) δ[ppm]: 0.800 (0.49), 0.804 (0.41), 0.817 (0.51), 0.823 (0.56), 0.904 (0.76), 0.907 (0.58), 1.037 (7.98), 1.055 (16.00), 1.072 (7.81), 1.162 (0.42), 1.232 (0.60), 1.276 (3.98), 1.306 (7.78), 1.323 (3.38), 1.382 (6.63), 1.804 (1.35), 1.906 (0.55), 2.068 (0.87), 2.324 (0.75), 2.329 (1.03), 2.334 (0.74), 2.454 (0.65), 2.520 (3.68), 2.525 (2.41), 2.667 (0.89), 2.671 (1.31), 2.676 (1.01), 2.944 (1.09), 3.226 (0.54), 3.407 (1.28), 3.419 (1.17), 3.425 (1.81), 3.437 (1.93), 3.442 (1.87), 3.454 (1.86), 3.472 (0.62), 4.346 (1.28), 4.359 (1.53), 4.372 (1.49), 4.664 (0.58), 4.702 (0.59), 4.811 (0.79), 4.829 (1.02), 4.844 (0.99), 7.120 (1.80), 7.380 (1.09), 7.399 (1.04), 7.526 (1.08), 7.539 (1.00), 8.027 (3.16), 9.125 (0.71), 12.219 (1.27).

### Intermediate 176

### tert-butyl 5-[4-(1H-benzimidazol-2-ylmethylamino)-8-bromo-pyrazolo[1,5-a][1,3,5]triazin-2-yl]-2,3,3a,4,6,6a-hexahydropyrrolo[2,3-c]pyrrole-1-carboxylate

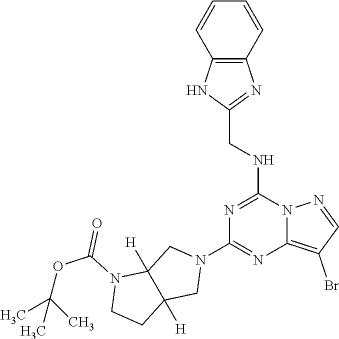 [SMILES: CC(C)(C)OC(=O)N1CCC2CN(c3nc(NCc4nc5ccccc5[nH]4)n4ncc(Br)c4n3)CC21]

N-[(1H-benzimidazol-2-yl)methyl]-8-bromo-2-(methanesulfonyl)pyrazolo[1,5-a][1,3,5]triazin-4-amine (Intermediate 9, 150 mg, 75% purity, 266 μmol) and tert-butyl rac-(3aR,6aR)-hexahydropyrrolo[3,4-b]pyrrole-1(2H)-carboxylate (73.5 mg, 346 μmol; CAS-RN:[1018443-32-7]) were provided in acetonitrile (2 mL), N,N-diisopropylethylamine (140 μL, 800 μmol; CAS-RN:[7087-68-5]) was added and the mixture was stirred for 20 h at 70° C. Water was added and the mixture was extracted with ethylacetate The combined organic layers were concentrated under reduced pressure. The residue was purified by flash chromatography (silica gel, dichloromethane/EtOH gradient) to give 160 mg (97% yield) of the title compound.

LC-MS (Method 2): Rₜ=1.29 min; MS (ESIpos): m/z=554 [M+H]⁺

¹H-NMR (400 MHz, DMSO-d6) δ[ppm]: 0.801 (0.67), 0.804 (0.59), 0.817 (0.65), 0.824 (0.76), 0.887 (1.40), 0.904 (2.77), 0.907 (1.00), 0.923 (1.53), 0.994 (0.82), 1.012 (0.81), 1.038 (9.09), 1.055 (16.00), 1.073 (10.07), 1.091 (0.90), 1.122 (3.41), 1.162 (1.03), 1.234 (0.62), 1.335 (2.49), 1.396 (6.66), 1.401 (4.84), 1.408 (4.46), 1.757 (0.45), 1.890 (0.78), 1.921 (0.89), 2.068 (3.84), 2.094 (0.57), 2.422 (0.96), 2.441 (1.08), 2.459 (0.96), 2.521 (4.82), 2.525 (3.23), 2.676 (0.97), 2.872 (0.40), 3.182 (0.55), 3.366 (1.00), 3.427 (1.53), 3.436 (1.57), 3.444 (1.50), 3.453 (1.40), 3.470 (0.55), 3.555 (0.79), 3.570 (0.77), 3.588 (0.94), 3.602 (0.82), 3.657 (0.75), 3.688 (0.63), 4.157 (0.65), 4.360 (0.91), 4.873 (1.63), 5.761 (3.10), 7.122 (2.30), 7.392 (0.88), 7.526 (1.09), 8.010 (6.57), 9.072 (0.80), 12.227 (0.92).

### Intermediate 177

### tert-butyl [1-(4-{[(1H-benzimidazol-2-yl)methyl]amino}-8-bromopyrazolo[1,5-a][1,3,5]triazin-2-yl)piperidin-4-yl]methylcarbamate

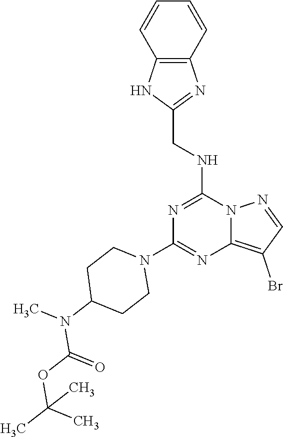 [SMILES: CN(C(=O)OC(C)(C)C)C1CCN(c2nc(NCc3nc4ccccc4[nH]3)n3ncc(Br)c3n2)CC1]

N-[(1H-benzimidazol-2-yl)methyl]-8-bromo-2-(methanesulfonyl)pyrazolo[1,5-a][1,3,5]triazin-4-amine (Intermediate 9, 150 mg, 89% purity, 316 μmol) and tert-butyl methyl(piperidin-4-yl)carbamate (88.1 mg, 411 μmol; CAS-RN:[108612-54-0]) were provided in acetonitrile (2 mL), N,N-diisopropylethylamine (170 μL, 950 μmol; CAS-RN:[7087-68-5]) was added and the mixture was stirred for 5 h at 70° C. Water was added and the mixture was extracted with ethylacetate The combined organic layers were concentrated under reduced pressure. The residue was purified by flash chromatography (silica gel, dichloromethane/EtOH gradient) to give 162 mg (87% yield) of the title compound.

LC-MS (Method 2): Rₜ=1.31 min; MS (ESIpos): m/z=556 [M+H]⁺

¹H-NMR (400 MHz, DMSO-d6) δ[ppm]: 0.904 (0.56), 1.037 (0.52), 1.055 (1.20), 1.073 (0.69), 1.378 (16.00), 1.397 (1.25), 1.413 (0.48), 2.067 (0.80), 2.488 (11.12), 2.520 (1.22), 2.525 (0.78), 2.747 (0.60), 4.841 (1.49), 4.855 (1.48), 5.761 (1.18), 7.101 (0.84), 7.106 (0.75), 7.110 (0.97), 7.117 (1.38), 7.124 (1.05), 7.128 (0.79), 7.133 (0.91), 7.380 (0.77), 7.387 (0.47), 7.396 (0.67), 7.401 (0.60), 7.519 (0.68), 7.524 (0.68), 7.539 (0.65), 8.021 (4.71), 9.113 (0.67), 12.243 (1.08).

### Intermediate 178

### 2-(chloromethyl)-5-fluoro-1-{[2-(trimethylsilyl)ethoxy]methyl}-1H-benzimidazole

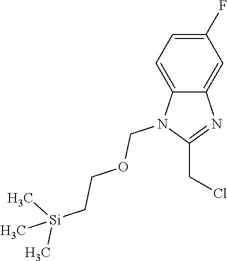 [SMILES: C[Si](C)(C)CCOCn1c(CCl)nc2cc(F)ccc21]

To a solution of 2-(chloromethyl)-5-fluoro-1H-benzimidazole (500 mg, 2.71 mmol; CAS-RN:[156144-42-2]) in tetrahydrofuran (10.0 mL) was added [2-(chloromethoxy)ethyl](trimethyl)silane (520 μL, 3.0 mmol; CAS-RN:[76513-69-4]) and N,N-diisopropylethylamine (860 μL, 4.9 mmol; CAS-RN:[7087-68-5]) at rt. The reaction mixture was stirred at rt for 1 h. The reaction mixture was concentrated under reduced pressure. The residue was purified by flash chromatography (silica gel, dichloromethane/EtOAc gradient) to give 623 mg (73% yield) of the title compound (50% impure with 2-(chloromethyl)-5-fluoro-1-{[2-(trimethylsilyl)ethoxy]methyl}-1H-benzimidazole).

LC-MS (Method 1): Rₜ=1.40 min; MS (ESIpos): m/z=315 [M+H]⁺

### Intermediate 179

### N-[(5-fluoro-1-{[2-(trimethylsilyl)ethoxy]methyl}-1H-benzimidazol-2-yl)methyl]-2-(morpholin-4-yl)-8-(trifluoromethyl)pyrazolo[1,5-a][1,3,5]triazin-4-amine

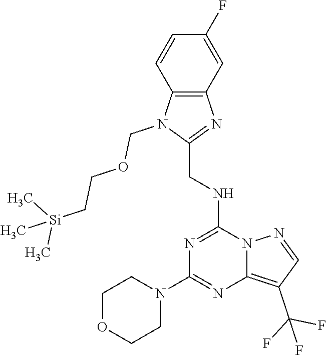 [SMILES: C[Si](C)(C)CCOCn1c(CNc2nc(N3CCOCC3)nc3c(C(F)(F)F)cnn23)nc2cc(F)ccc21]

To a solution of 2-(morpholin-4-yl)-8-(trifluoromethyl)pyrazolo[1,5-a][1,3,5]triazin-4-amine (Intermediate 131, 100 mg, 347 μmol) in N,N-dimethylformamide (10.0 mL) was added sodium hydride (16.7 mg, 60% purity, 416 μmol; CAS-RN:[7646-69-7]) at rt. After stirring at 60° C. for 30 min, 2-(chloromethyl)-5-fluoro-1-{[2-(trimethylsilyl)ethoxy]methyl}-1H-benzimidazole (50% impure with 2-(chloromethyl)-6-fluoro-1-{[2-(trimethylsilyl)ethoxy]methyl}-1H-benzimidazole) (Intermediate 178, 131 mg, 416 μmol) was added. The reaction mixture was stirred at 60° C. for 2 h. The reaction mixture was concentrated under reduced pressure to give a residue. The residue was purified by flash chromatography (silica gel, dichloromethane/EtOAc gradient) to give 178 mg (88% yield) of the title compound.

LC-MS (Method 1): Rₜ=1.52 min; MS (ESIpos): m/z=567 [M+H]⁺

### Intermediate 180

### 2-(chloromethyl)-4-fluoro-1-{[2-(trimethylsilyl)ethoxy]methyl}-1H-benzimidazole

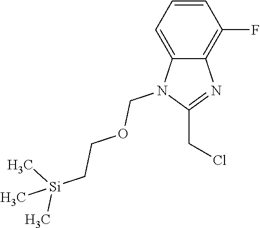 [SMILES: C[Si](C)(C)CCOCn1c(CCl)nc2c(F)cccc21]

To a solution of 2-(chloromethyl)-4-fluoro-1H-benzimidazole hydrogen chloride (1/1) (500 mg, 2.26 mmol; CAS-RN:[1258649-53-4]) in tetrahydrofuran (8.4 mL) was added [2-(chloromethoxy)ethyl](trimethyl)silane (440 μL, 2.5 mmol; CAS-RN:[76513-69-4]) and N,N-diisopropylethylamine (720 μL, 4.1 mmol; CAS-RN:[7087-68-5]) at rt. The reaction mixture was stirred for 18 h at rt. The reaction mixture was concentrated under reduced pressure. The residue was purified by flash chromatography (silica gel, dichloromethane/EtOAc gradient) to give 689 mg (90% yield) of the title compound (50% impure with 2-(chloromethyl)-7-fluoro-1-{[2-(trimethylsilyl)ethoxy]methyl}-1H-benzimidazole).

LC-MS (Method 1): Rₜ=1.43 min; MS (ESIpos): m/z=315 [M+H]⁺

### Intermediate 181

### N-[(4-fluoro-1-{[2-(trimethylsilyl)ethoxy]methyl}-1H-benzimidazol-2-yl)methyl]-2-(morpholin-4-yl)-8-(trifluoromethyl)pyrazolo[1,5-a][1,3,5]triazin-4-amine

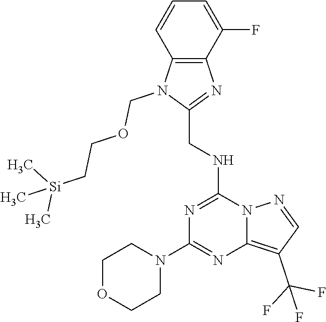 [SMILES: C[Si](C)(C)CCOCn1c(CNc2nc(N3CCOCC3)nc3c(C(F)(F)F)cnn23)nc2c(F)cccc21]

To a solution of 2-(morpholin-4-yl)-8-(trifluoromethyl)pyrazolo[1,5-a][1,3,5]triazin-4-amine (Intermediate 131, 100 mg, 347 μmol) in N,N-dimethylformamide (10.0 mL) was added sodium hydride (16.7 mg, 60% purity, 416 μmol; CAS-RN:[7646-69-7]) at rt. After stirring at 60° C. for 15 min, 2-(chloromethyl)-4-fluoro-1-{[2-(trimethylsilyl)ethoxy]methyl}-1H-benzimidazole (50% impure with 2-(chloromethyl)-7-fluoro-1-{[2-(trimethylsilyl)ethoxy]methyl}-1H-benzimidazole) (Intermediate 180, 120 mg, 382 μmol) was added. The reaction mixture was stirred for 3 days at rt. The reaction mixture was concentrated under reduced pressure to give a residue. The residue was purified by flash chromatography (silica gel, dichloromethane/EtOAc gradient) to give 177 mg (90% yield) of the title compound as a mixture of the two isomers.

LC-MS (Method 1): Rₜ=1.53/1.56 min; MS (ESIpos): m/z=567 [M+H]⁺

### Intermediate 182

### 5-chloro-2-(chloromethyl)-1-{[2-(trimethylsilyl)ethoxy]methyl}-1H-benzimidazole

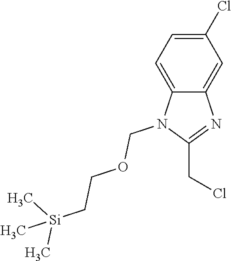 [SMILES: C[Si](C)(C)CCOCn1c(CCl)nc2cc(Cl)ccc21]

To a solution of 5-chloro-2-(chloromethyl)-1H-benzimidazole (500 mg, 2.49 mmol; CAS-RN:[20443-38-3]) in tetrahydrofuran (10.0 mL) was added [2-(chloromethoxy)ethyl](trimethyl)silane (480 μL, 2.7 mmol; CAS-RN:[76513-69-4]) and N,N-diisopropylethylamine (790 μL, 4.5 mmol; CAS-RN:[7087-68-5]) at rt. The reaction mixture was stirred over night at rt. The reaction mixture was concentrated under reduced pressure. The residue was purified by flash chromatography (silica gel, dichloromethane/EtOAc gradient) to give 766 mg (93% yield) of the title compound (50% impure with 6-chloro-2-(chloromethyl)-1-{[2-(trimethylsilyl)ethoxy]methyl}-1H-benzimidazole).

LC-MS (Method 1): Rₜ=1.49 min; MS (ESIpos): m/z=331 [M+H]⁺

### Intermediate 183

### N-[(5-chloro-1-{[2-(trimethylsilyl)ethoxy]methyl}-1H-benzimidazol-2-yl)methyl]-2-(morpholin-4-yl)-8-(trifluoromethyl)pyrazolo[1,5-a][1,3,5]triazin-4-amine

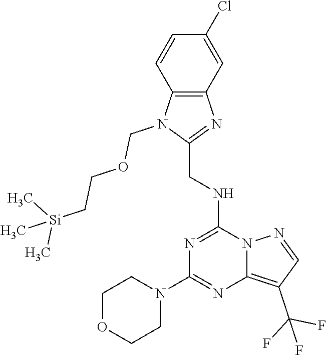 [SMILES: C[Si](C)(C)CCOCn1c(CNc2nc(N3CCOCC3)nc3c(C(F)(F)F)cnn23)nc2cc(Cl)ccc21]

To a solution of 2-(morpholin-4-yl)-8-(trifluoromethyl)pyrazolo[1,5-a][1,3,5]triazin-4-amine (Intermediate 131, 100 mg, 347 μmol) in N,N-dimethylformamide (10.0 mL) was added sodium hydride (16.7 mg, 60% purity, 416 μmol; CAS-RN:[7646-69-7]) at rt. After stirring at 60° C. for 15 min, 5-chloro-2-(chloromethyl)-1-{[2-(trimethylsilyl)ethoxy]methyl}-1H-benzimidazole (50% impure with 6-chloro-2-(chloromethyl)-1-{[2-(trimethylsilyl)ethoxy]methyl}-1H-benzimidazole) (Intermediate 182, 126 mg, 382 μmol) was added. The reaction mixture was stirred for 3 days at rt. The reaction mixture was concentrated under reduced pressure to give a residue. The residue was purified by flash chromatography (silica gel, dichloromethane/EtOAc gradient) to give 147 mg (73% yield) of the title compound as a mixture of the two isomers.

LC-MS (Method 1): Rₜ=1.58 min; MS (ESIpos): m/z=583 [M+H]⁺

### Intermediate 184

### 8-(3,6-dihydro-2H-pyran-4-yl)-N,N-bis[(4-methoxyphenyl)methyl]-2-(morpholin-4-yl)pyrazolo[1,5-a][1,3,5]triazin-4-amine

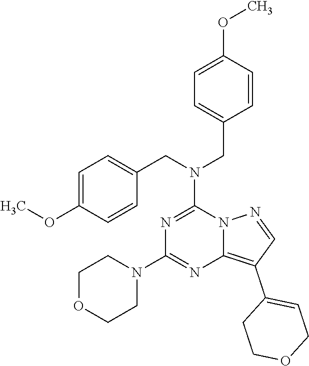 [SMILES: COc1ccc(CN(Cc2ccc(OC)cc2)c2nc(N3CCOCC3)nc3c(C4=CCOCC4)cnn23)cc1]

To a mixture of 8-iodo-N,N-bis[(4-methoxyphenyl)methyl]-2-(morpholin-4-yl)pyrazolo[1,5-a][1,3,5]triazin-4-amine (Intermediate 128, 1.00 g, 1.71 mmol) and 4-(4,4,5,5-tetramethyl-1,3,2-dioxaborolan-2-yl)-3,6-dihydro-2H-pyran (1.07 g, 5.12 mmol; CAS-RN:[287944-16-5]) in tetrahydrofuran (16 mL) were added methanesulfonato(2-dicyclohexylphosphino-2′,4′,6′-tri-i-propyl-1,1′-biphenyl)(2′-amino-1,1′-biphenyl-2-yl)palladium(II) (144 mg, 171 μmol; CAS-RN:[1445085-55-1]) and potassium phosphate (2.8 mL, 1.5 M, 4.3 mmol; CAS-RN:[7778-53-2]) in one portion. The reaction mixture was stirred at 60° C. for 4 h under nitrogen atmosphere. The reaction mixture was diluted with water and extracted with ethyl acetate. The organic phase was washed with brine, dried over anhydrous sodium sulfate and filtered. The filtrate was concentrated and purified by column chromatography on silica gel (200-300 mesh, petroleum ether: ethyl acetate=10:1) to give 1.10 g (crude) of the title compound as a red oil.

LC-MS (Method D): Rₜ=1.098 min; MS (ESIpos): m/z=543.4 [M+H]⁺.

### Intermediate 185

### N,N-bis[(4-methoxyphenyl)methyl]-2-(morpholin-4-yl)-8-(oxan-4-yl)pyrazolo[1,5-a][1,3,5]triazin-4-amine

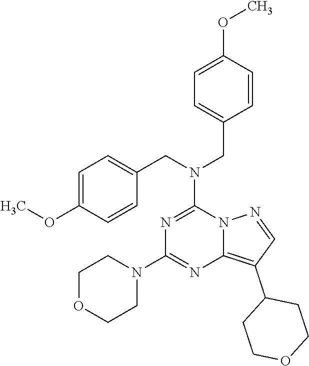 [SMILES: COc1ccc(CN(Cc2ccc(OC)cc2)c2nc(N3CCOCC3)nc3c(C4CCOCC4)cnn23)cc1]

To a solution of 8-(3,6-dihydro-2H-pyran-4-yl)-N,N-bis[(4-methoxyphenyl)methyl]-2-(morpholin-4-yl)pyrazolo[1,5-a][1,3,5]triazin-4-amine (Intermediate 184, 1.10 g, 2.03 mmol) in methanol (200 mL) was added palladium on carbon (60.0 mg, 10% purity). The reaction mixture was stirred at 40° C. for 16 h under hydrogen atmosphere. The reaction mixture was filtered and the filtrate was concentrated in reduced pressure to give 850 mg (77% yield) of the title compound as a yellow oil.

LC-MS (Method D): Rₜ=1.164 min; MS (ESIpos): m/z=545.4 [M+H]⁺.

### Intermediate 186

### 2-morpholino-8-tetrahydropyran-4-yl-pyrazolo[1,5-a][1,3,5]triazin-4-amine

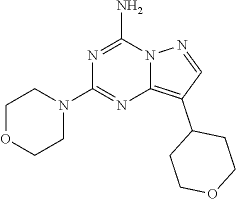 [SMILES: Nc1nc(N2CCOCC2)nc2c(C3CCOCC3)cnn12]

A solution of N,N-bis[(4-methoxyphenyl)methyl]-2-(morpholin-4-yl)-8-(oxan-4-yl)pyrazolo[1,5-a][1,3,5]triazin-4-amine (Intermediate 185, 650 mg, 1.19 mmol) in trifluoromethanesulfonic acid (10 mL) was stirred at 70° C. for 16 h. The reaction mixture was poured into ice water and basified to pH=8 by sodium carbonate. The mixture was extracted with ethyl acetate. The organic phase was washed with brine, dried over anhydrous sodium sulfate and filtered. The filtrate was concentrated and purified by column chromatography on silica gel (200-300 mesh, petroleum ether: ethyl acetate=5:1) to give 420 mg (crude) of the title compound as a yellow solid.

LC-MS (Method D): Rₜ=0.690 min; MS (ESIpos): m/z=305.2 [M+H]⁺.

### Intermediate 187

### N-[(4,5-difluoro-1-{[2-(trimethylsilyl)ethoxy]methyl}-1H-benzimidazol-2-yl)methyl]-2-(morpholin-4-yl)-8-(oxan-4-yl)pyrazolo[1,5-a][1,3,5]triazin-4-amine

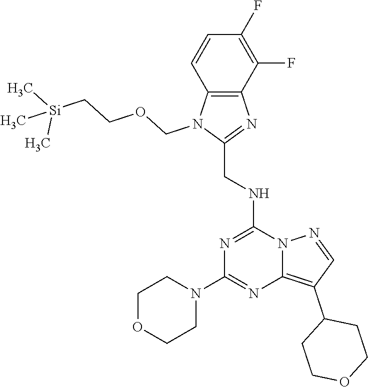 [SMILES: C[Si](C)(C)CCOCn1c(CNc2nc(N3CCOCC3)nc3c(C4CCOCC4)cnn23)nc2c(F)c(F)ccc21]

To a solution of 2-morpholino-8-tetrahydropyran-4-yl-pyrazolo[1,5-a][1,3,5]triazin-4-amine (Intermediate 186, 100 mg, 329 μmol) in N,N-dimethylformamide (3.0 mL) was added sodium hydride (23.7 mg, 60% purity, 591 μmol; CAS-RN:[7646-69-7]) at rt. After stirring at 60° C. for 15 minutes, 2-(chloromethyl)-6,7-difluoro-1-{[2-(trimethylsilyl)ethoxy]methyl}-1H-benzimidazole (50% impure with [2-(chloromethyl)-4,5-difluoro-benzimidazol-1-yl]methoxymethyl-trimethyl-silane) (Intermediate 158, 164 mg, 493 μmol) was added in one portion. The reaction mixture was stirred at rt for 16 h. The reaction mixture was concentrated in reduced pressure to give a residue. The residue was diluted with water and extracted with ethyl acetate. The organic phase was washed with brine, dried over anhydrous sodium sulfate and filtered. The filtrate was concentrated to give 150 mg (76% yield) of the title compound as a yellow oil.

LC-MS (Method D): Rₜ=1.064 min; MS (ESIpos): m/z=601.3 [M+H]⁺.

### Intermediate 188

### 2-(chloromethyl)-4-methoxy-1H-benzimidazole

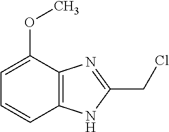

A solution of 3-methoxybenzene-1,2-diamine (5.00 g, 36.2 mmol) and chloroacetic acid (5.13 g, 54.3 mmol) in hydrochloric acid (5 M, 70 mL) was stirred at 100° C. for 16 h. The mixture was cooled to rt. Saturated sodium carbonate was added and adjusted to pH-7-8 by sodium carbonate. The black gum precipitated out, and then filtered. The filter cake was washed with water and concentrated to give 4.50 g (80% purity, 51% yield) of the title compound as a black gum.

LC-MS (Method C): Rₜ=0.14 min; MS (ESIpos): m/z=197 [M+H]⁺

### Intermediate 189

### 2-(chloromethyl)-4-methoxy-1-{[2-(trimethylsilyl)ethoxy]methyl}-1H-benzimidazole

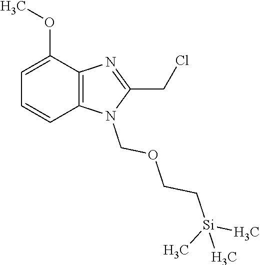 [SMILES: COc1cccc2c1nc(CCl)n2COCC[Si](C)(C)C]

To a solution of 2-(chloromethyl)-4-methoxy-1H-benzimidazole (Intermediate 188, 4.50 g, 80% purity, 18.3 mmol) and [2-(chloromethoxy)ethyl](trimethyl)silane (3.66 g, 22.0 mmol) in tetrahydrofuran (80 mL) was added N,N-diisopropylethylamine (4.73 g, 36.6 mmol) at rt. The reaction mixture was stirred at rt for 16 h. The mixture was concentrated in vacuum to give a residue. The residue was purified by silica gel chromatography (200-300 mesh, petroleum ether: ethyl acetate=10:1 then 3:1) to give 2.80 g (98% purity, 46% yield) of the title compound as a yellow oil.

LC-MS (Method C): Rₜ=0.90 min; MS (ESIpos): m/z=327 [M+H]⁺

### Intermediate 190

### N-[(4-methoxyphenyl)methyl]-N-[[4-methoxy-1-(2-trimethylsilylethoxymethyl)benzimidazol-2-yl]methyl]-2-morpholino-8-(2,2,2-trifluoroethyl)pyrazolo[1,5-a][1,3,5]triazin-4-amine

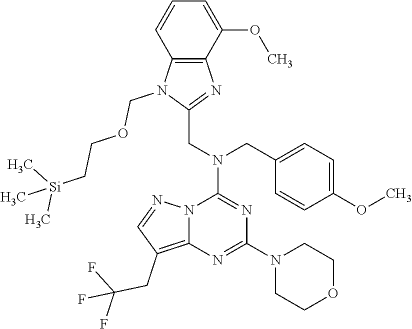

To a solution of N-[(4-methoxyphenyl)methyl]-2-(morpholin-4-yl)-8-(2,2,2-trifluoroethyl)pyrazolo[1,5-a][1,3,5]triazin-4-amine (Intermediate 169, 150 mg, 80% purity, 284 μmol) in N,N-dimethylformamide (4.0 mL) was added sodium hydride (22.7 mg, 60% purity, 568 μmol) at rt. After stirring at 60° C. for 20 min, 2-(chloromethyl)-4-methoxy-1-{[2-(trimethylsilyl)ethoxy]methyl}-1H-benzimidazole (Intermediate 189, 139 mg, 426 μmol) was added in one portion. The reaction mixture was stirred at rt for 16 h. The reaction mixture was poured into water. The mixture was extracted with ethyl acetate. The organic phase was washed with brine, dried over anhydrous sodium sulfate and filtered. The filtrate was concentrated to give 170 mg (66% purity, 55% yield) of the title compound as a yellow oil.

LC-MS (Method C): Rₜ=1.06 min; MS (ESIpos): m/z=714 [M+H]⁺

### Intermediate 191

### 8-ethenyl-N,N-bis[(4-methoxyphenyl)methyl]-2-(morpholin-4-yl)pyrazolo[1,5-a][1,3,5]triazin-4-amine

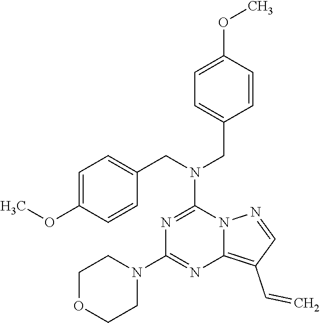 [SMILES: C=Cc1cnn2c(N(Cc3ccc(OC)cc3)Cc3ccc(OC)cc3)nc(N3CCOCC3)nc12]

To a solution of 8-iodo-N,N-bis[(4-methoxyphenyl)methyl]-2-(morpholin-4-yl)pyrazolo[1,5-a][1,3,5]triazin-4-amine (Intermediate 128, 2.00 g, 3.41 mmol) and potassium trifluoro(vinyl)borate(1-) (914 mg, 6.82 mmol) in tetrahydrofuran (20 mL) was added methanesulfonato(2-dicyclohexylphosphino-2′,4′,6′-tri-i-propyl-1,1′-biphenyl)(2′-amino-1,1′-biphenyl-2-yl)palladium(II) (144 mg, 171 μmol; CAS-RN:[1445085-55-1]) and potassium phosphate (6.8 mL, 1.5 M in water, 10 mmol; CAS-RN:[7778-53-2]), the mixture was stirred at 80° C. for 16 h. The mixture was filtered with a pad of celite and washed with ethyl acetate. The filtrate was concentrated under vacuum. The residue purified by column chromatography on silica gel (100-200 mesh, petroleum ether: ethyl acetate=10:1) to give 720 mg (43% yield) of the title compound as a white solid.

### Intermediate 192

### 8-(2,2-difluorocyclopropyl)-N,N-bis[(4-methoxyphenyl)methyl]-2-(morpholin-4-yl)pyrazolo[1,5-a][1,3,5]triazin-4-amine

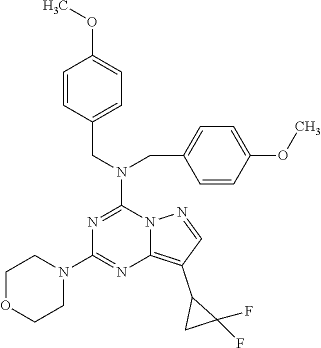 [SMILES: COc1ccc(CN(Cc2ccc(OC)cc2)c2nc(N3CCOCC3)nc3c(C4CC4(F)F)cnn23)cc1]

To a solution of 8-ethenyl-N,N-bis[(4-methoxyphenyl)methyl]-2-(morpholin-4-yl)pyrazolo[1,5-a][1,3,5]triazin-4-amine (Intermediate 191, 450 mg, 925 μmol) in tetrahydrofuran (9.0 mL) was added sodium iodide (27.7 mg, 185 μmol; CAS-RN:[7681-82-5]), then trimethyl(trifluoromethyl)silane (2.7 mL, 18 mmol; CAS-RN:[81290-20-2]) was added to the mixture and the mixture was stirred at 110° C. for 2 h in microwave under nitrogen. The reaction was cooled to rt and evaporated under reduced pressure to give a residue. The residue was purified by column chromatography on silica gel (100-200 mesh, petroleum ether: ethyl acetate=10:1, then 5:1, then 2:1) to give 1.35 g (86% purity, crude) of the title compound as a yellow oil.

LC-MS (Method C): Rₜ=1.023 min; MS (ESIpos): m/z=537.1 [M+H]⁺

### Intermediate 193

### 8-(2,2-difluorocyclopropyl)-2-(morpholin-4-yl)pyrazolo[1,5-a][1,3,5]triazin-4-amine

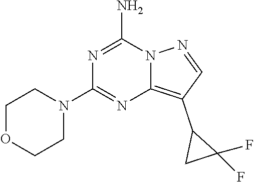 [SMILES: Nc1nc(N2CCOCC2)nc2c(C3CC3(F)F)cnn12]

A solution of 8-(2,2-difluorocyclopropyl)-N,N-bis[(4-methoxyphenyl)methyl]-2-(morpholin-4-yl)pyrazolo[1,5-a][1,3,5]triazin-4-amine (Intermediate 192, 1.15 g, 86% purity, 1.84 mmol) in trifluoromethanesulfonic acid (18 mL, 200 mmol; CAS-RN:[1493-13-6]) was stirred at 70° C. for 16 h. The reaction mixture was cooled to rt. The mixture was poured into ice slowly and then adjusted to pH 8-9 by sodium hydroxide at 0° C. The mixture was extracted with dichloromethane. The organic layer was evaporated under reduced pressure to give a residue. The residue was purified by chromatography (100-200 mesh, petroleum ether: ethyl acetate=9:1, then 4:1, then 2:1, thn 1:1) to give 550 mg (crude) of the title compound as a brown solid

LC-MS (Method C): Rₜ=0.706 min; MS (ESIpos): m/z=297.0 [M+H]⁺

### Intermediate 194

### 8-(2,2-difluorocyclopropyl)-N-[(4,5-difluoro-1-{[2-(trimethylsilyl)ethoxy]methyl}-1H-benzimidazol-2-yl)methyl]-2-(morpholin-4-yl)pyrazolo[1,5-a][1,3,5]triazin-4-amine-8-(2,2-difluorocyclopropyl)-N-[(6,7-difluoro-1-{[2-(trimethylsilyl)ethoxy]methyl}-1H-benzimidazol-2-yl)methyl]-2-(morpholin-4-yl)pyrazolo[1,5-a][1,3,5]triazin-4-amine (1/1)

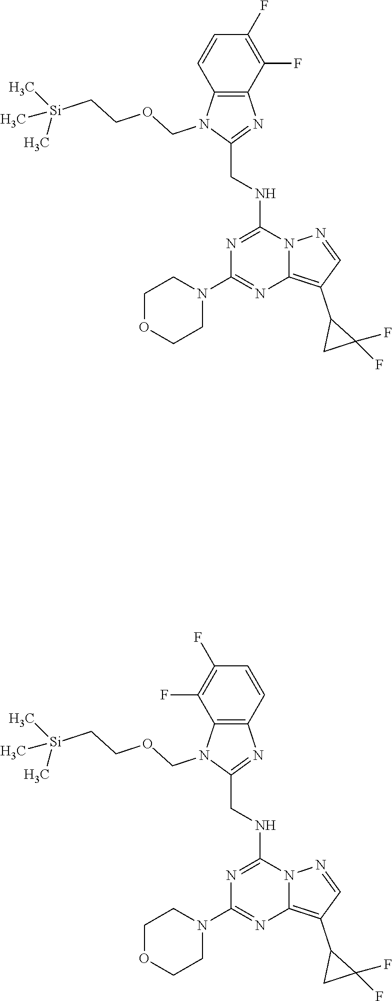 [SMILES: C[Si](C)(C)CCOCn1c(CNc2nc(N3CCOCC3)nc3c(C4CC4(F)F)cnn23)nc2c(F)c(F)ccc21 ; C[Si](C)(C)CCOCn1c(CNc2nc(N3CCOCC3)nc3c(C4CC4(F)F)cnn23)nc2ccc(F)c(F)c21]

To a solution of 8-(2,2-difluorocyclopropyl)-2-(morpholin-4-yl)pyrazolo[1,5-a][1,3,5]triazin-4-amine (Intermediate 193, 100 mg, 338 μmol) in N,N-dimethylformamide (2.0 mL) was added sodium hydride (16.2 mg, 60% purity, 405 μmol; CAS-RN:[7646-69-7]) at rt. After stirring at 60° C. for 15 min, 2-(chloromethyl)-6,7-difluoro-1-{[2-(trimethylsilyl)ethoxy]methyl}-1H-benzimidazole (50% impure with [2-(chloromethyl)-4,5-difluoro-benzimidazol-1-yl]methoxymethyl-trimethyl-silane) (Intermediate 158, 135 mg, 405 μmol) was added in one portion. The reaction mixture was stirred at rt for 16 h. The reaction mixture was concentrated under reduced pressure to give a residue. The residue was diluted with water and extracted with ethyl acetate. The organic phase was washed with brine, dried over anhydrous sodium sulfate and filtered. The filtrate was concentrated to give 200 mg (45% purity, 45% yield) of the title compound as a brown oil.

LC-MS (Method C): Rₜ=1.017 min; MS (ESIpos): m/z=593.2 [M+H]⁺

### Intermediate 195

### 8-(2,2-difluorocyclopropyl)-2-(morpholin-4-yl)-N-[(1-{[2-(trimethylsilyl)ethoxy]methyl}-1H-benzimidazol-2-yl)methyl]pyrazolo[1,5-a][1,3,5]triazin-4-amine

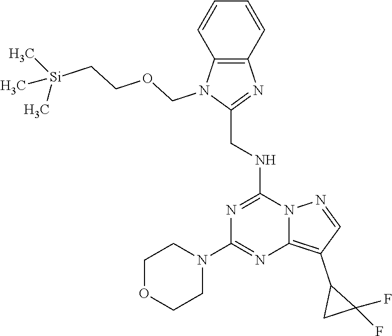 [SMILES: C[Si](C)(C)CCOCn1c(CNc2nc(N3CCOCC3)nc3c(C4CC4(F)F)cnn23)nc2ccccc21]

To a solution of 8-(2,2-difluorocyclopropyl)-2-(morpholin-4-yl)pyrazolo[1,5-a][1,3,5]triazin-4-amine (Intermediate 193, 100 mg, 338 μmol) in N,N-dimethylformamide (2.0 mL) was added sodium hydride (16.2 mg, 60% purity, 405 μmol; CAS-RN:[7646-69-7]) at rt. After stirring at 60° C. for 15 min, 2-(chloromethyl)-1-{[2-(trimethylsilyl)ethoxy]methyl}-1H-benzimidazole (Intermediate 130, 120 mg, 405 μmol) was added in one portion. The reaction mixture was stirred at rt for 16 h. The reaction mixture was concentrated under reduced pressure to give a residue. The residue was diluted with water and extracted with ethyl acetate. The organic phase was washed with brine, dried over anhydrous sodium sulfate and filtered. The filtrate was concentrated to give 180 mg (51% purity, 49% yield) of the title compound as a brown oil.

LC-MS (Method C): Rₜ=0.88 min; MS (ESIpos): m/z=557.2 [M+H]⁺

### Intermediate 196

### tert-butyl 3-(4-{bis[(4-methoxyphenyl)methyl]amino}-8-iodopyrazolo[1,5-a][1,3,5]triazin-2-yl)-3,8-diazabicyclo[3.2.1]octane-8-carboxylate

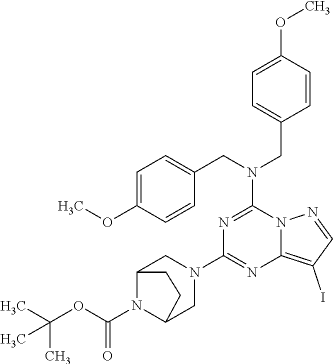 [SMILES: COc1ccc(CN(Cc2ccc(OC)cc2)c2nc(N3CC4CCC(C3)N4C(=O)OC(C)(C)C)nc3c(I)cnn23)cc1]

To a solution of 8-iodo-2-(methanesulfonyl)-N,N-bis[(4-methoxyphenyl)methyl]pyrazolo[1,5-a][1,3,5]triazin-4-amine (Intermediate 127, 8.00 g, 13.8 mmol) and tert-butyl 3,8-diazabicyclo[3.2.1]octane-8-carboxylate (4.40 g, 20.7 mmol; CAS-RN:[149771-44-8]) in acetonitrile (100 mL) was added N,N-diisopropylethylamine (4.8 mL, 27.6 mmol; CAS-RN:[7087-68-5]) at rt. The reaction mixture was stirred at 70° C. for 1 day and concentrated under reduced pressure. The residue was purified by flash chromatography (silica gel, dichloromethane/Ethyl acetate gradient) to give 8.26 g (84% yield) of the title compound.

LC-MS (Method 2): Rₜ=1.81 min; MS (ESIpos): m/z=712 [M+H]⁺

¹H-NMR (400 MHz, DMSO-d6) δ[ppm]: 1.154 (0.76), 1.172 (1.55), 1.190 (0.74), 1.414 (11.40), 1.988 (2.44), 2.518 (1.03), 2.523 (0.67), 3.286 (0.50), 3.722 (16.00), 3.736 (1.02), 4.017 (0.57), 4.035 (0.58), 6.875 (2.64), 6.879 (0.91), 6.891 (0.98), 6.896 (2.97), 6.904 (0.41), 7.226 (2.65), 7.248 (2.32), 7.950 (3.64).

### Intermediate 197

### tert-butyl 3-[4-{bis[(4-methoxyphenyl)methyl]amino}-8-(trifluoromethyl)pyrazolo[1,5-a][1,3,5]triazin-2-yl]-3,8-diazabicyclo[3.2.1]octane-8-carboxylate

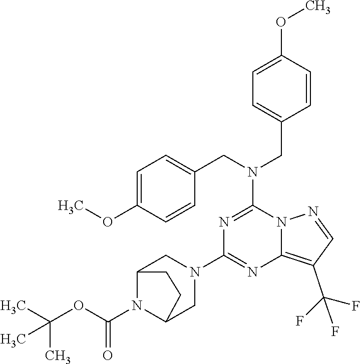 [SMILES: COc1ccc(CN(Cc2ccc(OC)cc2)c2nc(N3CC4CCC(C3)N4C(=O)OC(C)(C)C)nc3c(C(F)(F)F)cnn23)cc1]

To a suspension of tert-butyl 3-(4-{bis[(4-methoxyphenyl)methyl]amino}-8-iodopyrazolo[1,5-a][1,3,5]triazin-2-yl)-3,8-diazabicyclo[3.2.1]octane-8-carboxylate (Intermediate 196, 4.00 g, 5.62 mmol) and copper(I) iodide (4.28 g, 22.5 mmol) in N,N-dimethylformamide (55 mL) was added methyl 2,2-difluoro-2-(fluorosulfonyl)acetate (2.9 mL, 22.5 mmol; CAS-RN:[680-15-9]) at rt under an argon atmosphere. The reaction mixture was stirred for 2 h at 80° C., for 1 h at 90° C., for 2 h at 100° C. and for 3 days at room temperature. The reaction mixture was concentrated in vacuo and the residue was purified by flash chromatography (silica gel, dichloromethane/Ethyl acetate gradient) to give 2.61 g (71% yield) of the title compound.

LC-MS (Method 2): Rₜ=1.79 min; MS (ESIpos): m/z=654 [M+H]⁺

¹H-NMR (400 MHz, DMSO-d6) δ[ppm]: 1.154 (0.73), 1.172 (1.61), 1.190 (0.84), 1.410 (15.19), 1.987 (3.08), 2.518 (0.46), 3.330 (16.00), 3.743 (0.43), 4.017 (0.68), 4.034 (0.67), 6.873 (0.40), 6.881 (2.91), 6.903 (3.23), 7.249 (3.19), 7.254 (1.12), 7.271 (2.88), 8.205 (2.14).

### Intermediate 198

### 2-(3,8-diazabicyclo[3.2.1]octan-3-yl)-8-(trifluoromethyl)pyrazolo[1,5-a][1,3,5]triazin-4-amine

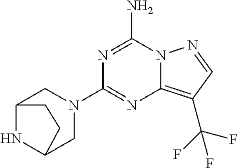 [SMILES: Nc1nc(N2CC3CCC(C2)N3)nc2c(C(F)(F)F)cnn12]

A solution of tert-butyl 3-[4-{bis[(4-methoxyphenyl)methyl]amino}-8-(trifluoromethyl)pyrazolo[1,5-a][1,3,5]triazin-2-yl]-3,8-diazabicyclo[3.2.1]octane-8-carboxylate (Intermediate 197, 200 mg, 306 μmol) in trifluoromethanesulfonic acid (810 μL, 9.14 mmol; CAS-RN:[1493-13-6]) was stirred at 70° C. for 90 minutes. The solution was added to a saturated, aqueous solution of sodium bicarbonate and then extracted with ethyl acetate. The organic phase was filtered over a hydrophobic filter and concentrated under reduced pressure. The residue was purified by flash chromatography twice using silica gel (dichloromethane/ethanol gradient) to give 45.0 mg (47% yield) of the title compound.

LC-MS (Method 2): Rₜ=0.97 min; MS (ESIpos): m/z=314 [M+H]⁺

¹H-NMR (400 MHz, DMSO-d6) δ[ppm]: 0.798 (0.66), 0.814 (0.77), 0.821 (0.77), 0.886 (0.41), 0.904 (0.88), 0.922 (0.44), 1.035 (0.60), 1.052 (1.43), 1.070 (0.74), 1.230 (0.99), 1.472 (1.18), 1.491 (5.32), 1.509 (5.65), 1.523 (2.25), 1.547 (0.88), 1.560 (0.88), 1.598 (3.76), 1.610 (3.49), 1.620 (3.18), 2.193 (1.23), 2.318 (0.91), 2.322 (1.26), 2.327 (1.67), 2.332 (1.23), 2.336 (0.60), 2.412 (1.10), 2.518 (6.48), 2.523 (4.17), 2.660 (0.52), 2.665 (1.15), 2.669 (1.67), 2.673 (1.18), 2.678 (0.55), 2.954 (2.25), 3.435 (6.15), 4.221 (4.67), 4.251 (4.50), 5.758 (7.57), 8.096 (1.73), 8.135 (16.00), 8.317 (0.82), 8.333 (0.77), 8.358 (0.96).

### Intermediate 199

### benzyl 3-[4-amino-8-(trifluoromethyl)pyrazolo[1,5-a][1,3,5]triazin-2-yl]-3,8-diazabicyclo[3.2.1]octane-8-carboxylate

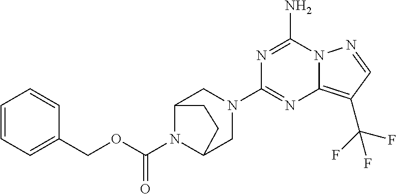 [SMILES: Nc1nc(N2CC3CCC(C2)N3C(=O)OCc2ccccc2)nc2c(C(F)(F)F)cnn12]

To a mixture of 2-(3,8-diazabicyclo[3.2.1]octan-3-yl)-8-(trifluoromethyl)pyrazolo[1,5-a][1,3,5]triazin-4-amine (Intermediate 198, 44.0 mg, 140 μmol) and sodium bicarbonate (35.4 mg, 421 μmol) in 620 μL of water and 620 μL of tetrahydrofuran was added benzyl carbonochloridate (40 μL, 280 μmol; CAS-RN:[501-53-1]) at 0° C. The mixture was stirred for 30 min at room temperature and concentrated under reduced pressure. The residue was purified by flash chromatography (silica gel, dichloromethane/ethyl acetate gradient) to give 45.0 mg (72% yield) of the title compound.

LC-MS (Method 2): Rₜ=1.37 min; MS (ESIpos): m/z=448 [M+H]⁺

¹H-NMR (400 MHz, DMSO-d6) δ[ppm]: 1.154 (0.51), 1.172 (1.03), 1.190 (0.55), 1.232 (1.40), 1.578 (2.09), 1.596 (2.24), 1.825 (1.56), 1.987 (1.71), 2.318 (0.63), 2.322 (1.03), 2.327 (1.37), 2.332 (1.01), 2.336 (0.43), 2.518 (5.53), 2.523 (3.42), 2.660 (0.41), 2.664 (0.96), 2.669 (1.37), 2.673 (0.99), 2.678 (0.43), 3.050 (1.11), 4.354 (2.17), 4.412 (2.82), 4.444 (2.69), 5.129 (10.47), 7.298 (0.41), 7.309 (0.99), 7.315 (0.67), 7.319 (2.05), 7.327 (0.99), 7.331 (2.21), 7.341 (1.64), 7.350 (0.58), 7.357 (0.75), 7.360 (0.79), 7.365 (0.65), 7.379 (16.00), 7.390 (15.13), 8.137 (0.53), 8.176 (8.49), 8.204 (0.60), 8.480 (0.53).

### Intermediate 200

### benzyl 3-[4-{[(4-fluoro-1-{[2-(trimethylsilyl)ethoxy]methyl}-1H-benzimidazol-2-yl)methyl]amino}-8-(trifluoromethyl)pyrazolo[1,5-a][1,3,5]triazin-2-yl]-3,8-diazabicyclo[3.2.1]octane-8-carboxylate

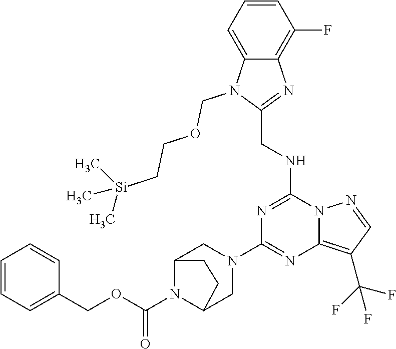 [SMILES: C[Si](C)(C)CCOCn1c(CNc2nc(N3CC4CCC(C3)N4C(=O)OCc3ccccc3)nc3c(C(F)(F)F)cnn23)nc2c(F)cccc21]

To a solution of benzyl 3-[4-amino-8-(trifluoromethyl)pyrazolo[1,5-a][1,3,5]triazin-2-yl]-3,8-diazabicyclo[3.2.1]octane-8-carboxylate (Intermediate 199, 178 mg, 398 μmol) in N,N-dimethylformamide (11.0 mL) was added sodium hydride (25.5 mg, 60% purity, 637 μmol; CAS-RN:[7646-69-7]) at room temperature. After stirring at 30° C. for 15 min, 2-(chloromethyl)-4-fluoro-1-{[2-(trimethylsilyl)ethoxy]methyl}-1H-benzimidazole (Intermediate 180, 150 mg, 477 μmol) was added and the reaction mixture was stirred for 45 minutes at room temperature. The reaction mixture was concentrated under reduced pressure and the residue was purified by flash chromatography (silica gel, dichloromethane/EtOAc gradient) to give 261 mg (90% yield) of the title compound.

LC-MS (Method 1): Rₜ=1.65 min; MS (ESIpos): m/z=726 [M+H]⁺

¹H-NMR (400 MHz, DMSO-d6) δ[ppm]: −0.205 (16.00), −0.187 (0.91), 0.393 (0.59), 0.416 (0.58), 1.232 (0.48), 1.282 (0.46), 1.300 (0.43), 2.518 (4.09), 2.523 (2.61), 2.926 (0.47), 2.958 (0.44), 3.316 (0.64), 3.353 (0.69), 4.336 (0.50), 4.368 (0.54), 4.385 (0.48), 4.416 (0.48), 4.944 (1.05), 5.097 (3.77), 5.130 (0.42), 5.629 (0.81), 5.670 (0.86), 7.221 (0.64), 7.233 (0.64), 7.320 (0.45), 7.347 (1.00), 7.362 (3.56), 7.379 (1.32), 7.390 (0.96), 7.449 (1.19), 7.469 (0.98), 8.224 (3.13), 9.376 (0.65).

### Intermediate 201

### N-[(4-fluoro-1-{[2-(trimethylsilyl)ethoxy]methyl}-1H-benzimidazol-2-yl)methyl]-2-(8-methyl-3,8-diazabicyclo[3.2.1]octan-3-yl)-8-(trifluoromethyl)pyrazolo[1,5-a][1,3,5]triazin-4-amine

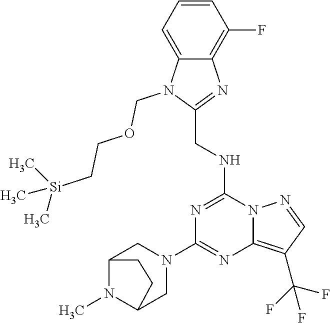 [SMILES: CN1C2CCC1CN(c1nc(NCc3nc4c(F)cccc4n3COCC[Si](C)(C)C)n3ncc(C(F)(F)F)c3n1)C2]

To a solution of benzyl 3-[4-{[(4-fluoro-1-{[2-(trimethylsilyl)ethoxy]methyl}-1H-benzimidazol-2-yl)methyl]amino}-8-(trifluoromethyl)pyrazolo[1,5-a][1,3,5]triazin-2-yl]-3,8-diazabicyclo[3.2.1]octane-8-carboxylate (Intermediate 200, 260 mg, 358 μmol) in 9.0 mL methanol under argon was added formaldehyde in water (53 μL, 37% purity, 720 μmol) and palladium (75.0 mg, 10% on activated carbon). The argon atmosphere was replaced by hydrogen and the mixture was stirred for 4 hours at room temperature. The atmosphere was replaced by air and the mixture was stirred for further 3 days at room temperature. The mixture was filtered and concentrated under reduced pressure. The residue was purified by flash chromatography (silica gel, dichloromethane/ethanol gradient) to give 189 mg (87% yield) of the title compound.

LC-MS (Method 1): Rₜ=1.15 min; MS (ESIpos): m/z=606 [M+H]⁺

¹H-NMR (400 MHz, DMSO-d6) δ[ppm]: −0.189 (1.01), −0.180 (16.00), −0.172 (0.54), −0.167 (0.25), −0.138 (0.27), 0.445 (0.24), 0.466 (0.24), 1.233 (0.19), 2.099 (2.61), 2.518 (1.03), 2.523 (0.71), 2.824 (0.16), 2.853 (0.28), 3.366 (0.19), 4.135 (0.18), 4.165 (0.18), 4.915 (0.18), 4.930 (0.18), 4.946 (0.19), 4.957 (0.18), 5.640 (0.32), 5.677 (0.31), 7.003 (0.19), 7.022 (0.24), 7.031 (0.21), 7.050 (0.23), 7.225 (0.28), 7.237 (0.29), 7.245 (0.16), 7.456 (0.47), 7.475 (0.39), 8.188 (1.02), 9.260 (0.26).

### Intermediate 202

### methyl N-[2-(morpholin-4-yl)-8-(trifluoromethyl)pyrazolo[1,5-a][1,3,5]triazin-4-yl]glycinate

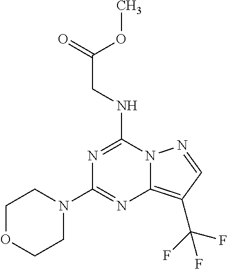 [SMILES: COC(=O)CNc1nc(N2CCOCC2)nc2c(C(F)(F)F)cnn12]

To a solution of 2-(morpholin-4-yl)-8-(trifluoromethyl)pyrazolo[1,5-a][1,3,5]triazin-4-amine (Intermediate 131, 900 mg, 3.12 mmol) in 5 mL of DMF was added sodium hydride (74.9 mg, 60% in mineral oil, 1.87 mmol; CAS-RN:[7646-69-7]) and the mixture was stirred for 20 min at 60° C. Then methyl chloroacetate (300 μL, 3.44 mmol) was added and the mixture was stirred for 20 hours at room temperature. The reaction mixture was quenched with water and extracted with ethyl acetate. The combined organic layers were washed with brine, filtered over a hydrophobic filter and concentrated. The residue was purified by flash chromatography (silica gel, dichloromethane/ethanol gradient) and subsequent stirring of the crude product with methyltertbutylether. The precipitate was filtered off and dried to give 383 mg (34% yield) of the title compound.

LC-MS (Method 2): Rₜ=1.15 min; MS (ESIpos): m/z=361 [M+H]⁺

¹H-NMR (400 MHz, DMSO-d6) δ[ppm]: 3.618 (2.08), 3.628 (4.14), 3.639 (3.58), 3.664 (16.00), 3.695 (3.32), 3.706 (3.73), 4.184 (4.60), 8.252 (4.23), 9.131 (1.34).

### Intermediate 203

### N-[2-(morpholin-4-yl)-8-(trifluoromethyl)pyrazolo[1,5-a][1,3,5]triazin-4-yl]glycine

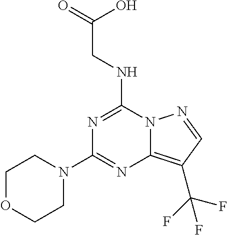 [SMILES: O=C(O)CNc1nc(N2CCOCC2)nc2c(C(F)(F)F)cnn12]

To a solution of methyl N-[2-(morpholin-4-yl)-8-(trifluoromethyl)pyrazolo[1,5-a][1,3,5]triazin-4-yl]glycinate (Intermediate 202, 380 mg, 1.05 mmol) in 5.0 mL of tetrahydrofuran and 2.0 mL of ethanol an aqueous solution of lithium hydroxide (2.6 mL, 1.0 M, 2.60 mmol; CAS-RN:[1310-65-2]) was added. The mixture was stirred for 72 hours at room temperature. Water was added and the mixture was acidified with an aqueous solution of citric acid (10%) till pH 3-4 was achieved. The formed precipitate was filtered off, washed with water and dried to give 319 mg (79% yield) of the title compound.

LC-MS (Method 2): Rₜ=0.63 min; MS (ESIpos): m/z=347 [M+H]⁺

¹H-NMR (400 MHz, DMSO-d6) δ[ppm]: 1.102 (14.43), 1.154 (0.44), 1.166 (0.41), 1.171 (0.89), 1.189 (0.46), 1.230 (0.48), 1.250 (0.43), 1.268 (0.87), 1.907 (0.43), 1.987 (1.15), 2.322 (0.74), 2.327 (1.02), 2.332 (0.78), 2.522 (3.88), 2.664 (0.78), 2.669 (1.07), 2.673 (0.80), 3.072 (4.57), 3.620 (10.43), 3.630 (15.37), 3.642 (14.34), 3.711 (14.32), 3.724 (16.00), 3.734 (8.88), 4.084 (10.40), 4.099 (10.43), 5.757 (1.02), 8.173 (3.03), 8.184 (0.70), 8.221 (0.76), 8.243 (15.02), 8.948 (2.13), 8.963 (4.66), 8.978 (2.18), 12.860 (0.52).

### Intermediate 204

### 2-{[2-(morpholin-4-yl)-8-(trifluoromethyl)pyrazolo[1,5-a][1,3,5]triazin-4-yl]amino}acetohydrazide

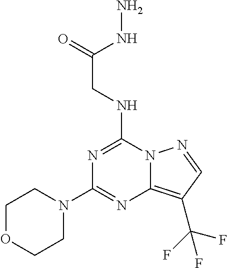 [SMILES: NNC(=O)CNc1nc(N2CCOCC2)nc2c(C(F)(F)F)cnn12]

N-[2-(morpholin-4-yl)-8-(trifluoromethyl)pyrazolo[1,5-a][1,3,5]triazin-4-yl]glycine (Intermediate 203, 315 mg, 819 μmol) was dissolved in 8.0 mL of THF, di-1H-imidazol-1-ylmethanone (266 mg, 1.64 mmol; CAS-RN:[530-62-1]) was added and the reaction mixture was stirred under reflux for 6 h. The solution was cooled to rt and hydrazine in THF (4.1 mL, 1.0 M, 4.1 mmol) was added dropwise. The reaction mixture was stirred for 18 h at room temperature. Water was added and the mixture was extracted with ethyl acetate. The combined organic layers were washed with brine, filtered and concentrated. The residue was stirred in methyltertbutylether, the precipitate was filtered off, washed with methyltertbutylether and dried to give 244 mg of the title compound (66% yield).

LC-MS (Method 2): Rₜ=0.90 min; MS (ESIpos): m/z=361 [M+H]⁺

¹H-NMR (400 MHz, DMSO-d6) δ[ppm]: 1.154 (2.21), 1.172 (4.53), 1.189 (2.21), 1.266 (0.40), 1.757 (0.65), 1.987 (7.98), 2.322 (0.88), 2.326 (1.20), 2.331 (0.88), 2.518 (7.94), 2.522 (5.37), 2.664 (0.95), 2.668 (1.26), 2.673 (0.91), 3.599 (0.99), 3.620 (7.83), 3.629 (15.47), 3.642 (14.97), 3.707 (13.09), 3.719 (15.12), 3.730 (8.97), 3.986 (11.87), 4.016 (2.00), 4.034 (1.77), 4.052 (0.55), 4.240 (5.03), 4.357 (0.67), 4.499 (1.24), 7.633 (0.42), 8.141 (0.46), 8.174 (0.88), 8.194 (0.51), 8.209 (16.00), 8.217 (1.94), 8.669 (0.63), 8.811 (1.71), 9.204 (4.74).

### Intermediate 205

### N-[(5-chloro-4-fluoro-1H-benzimidazol-2-yl)methyl]-8-cyclopropyl-2-(methylsulfanyl)pyrazolo[1,5-a][1,3,5]triazin-4-amine

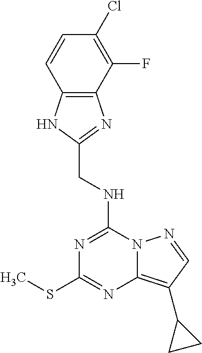 [SMILES: CSc1nc(NCc2nc3c(F)c(Cl)ccc3[nH]2)n2ncc(C3CC3)c2n1]

To dichloromethane (10 mL) was added 1-(5-chloro-4-fluoro-1H-benzimidazol-2-yl)methanamine dihydrochloride (500 mg, 1.83 mmol), N,N-diisopropylethylamine (910 μL, 5.24 mmol; CAS-RN:[7087-68-5]) and 4-chloro-8-cyclopropyl-2-(methylsulfanyl)pyrazolo[1,5-a][1,3,5]triazine (Intermediate 55, 421 mg, 1.75 mmol). The reaction mixture was stirred over night at rt. The mixture was concentrated under reduced pressure and treated with water. The precipitate was filtered off, washed with water and dried at 60° C. under reduced pressure to give 701 mg (95% yield) of the title compound.

LC-MS (Method 1): Rₜ=1.28 min; MS (ESIpos): m/z=404 [M+H]⁺

¹H-NMR (400 MHz, DMSO-d6) δ[ppm]: 0.809 (0.73), 0.822 (2.08), 0.827 (2.38), 0.834 (3.34), 0.840 (2.32), 0.847 (1.33), 0.851 (0.74), 0.862 (1.84), 0.868 (2.20), 0.874 (1.59), 0.878 (0.87), 0.882 (1.45), 0.890 (2.32), 0.895 (1.50), 0.908 (0.63), 1.242 (2.70), 1.254 (2.76), 1.854 (0.72), 1.862 (0.74), 1.871 (0.71), 1.875 (1.14), 1.883 (0.47), 1.888 (0.70), 1.896 (0.66), 2.394 (16.00), 2.518 (2.15), 2.523 (1.29), 4.903 (2.36), 4.917 (2.36), 7.270 (2.08), 7.275 (2.31), 7.280 (3.98), 7.941 (3.93), 9.300 (0.40), 9.314 (0.73), 12.800 (0.77).

### Intermediate 206

### N-[(5-chloro-4-fluoro-1H-benzimidazol-2-yl)methyl]-8-cyclopropyl-2-(methanesulfonyl)pyrazolo[1,5-a][1,3,5]triazin-4-amine

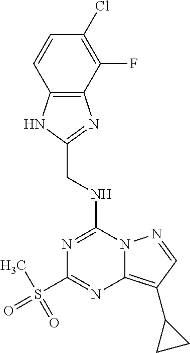 [SMILES: CS(=O)(=O)c1nc(NCc2nc3c(F)c(Cl)ccc3[nH]2)n2ncc(C3CC3)c2n1]

To N-[(5-chloro-4-fluoro-1H-benzimidazol-2-yl)methyl]-8-cyclopropyl-2-(methylsulfanyl)pyrazolo[1,5-a][1,3,5]triazin-4-amine (Intermediate 205, 700 mg, 1.73 mmol) in dichloromethane (20 mL) at 0° C. was added 3-chlorobenzenecarboperoxoic acid (1.17 g, 77% purity, 5.20 mmol; CAS-RN:[937-14-4]). The reaction mixture was stirred for 72 h at rt. Aqueous saturated sodium bicarbonate solution was added till a basic pH was reached and the mixture was stirred for 20 min at room temperature. The organic layer was separated, the aqueous layer was extracted with dichloromethane and the combined organic layers were dried over a hydrophobic filter and concentrated to give 815 mg of a crude product of the title compound which was used without further purification.

### Intermediate 207

### benzyl (3S)-4-(4-{bis[(4-methoxyphenyl)methyl]amino}-8-iodopyrazolo[1,5-a][1,3,5]triazin-2-yl)-3-methylpiperazine-1-carboxylate

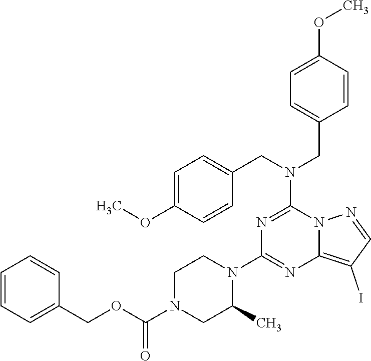 [SMILES: COc1ccc(CN(Cc2ccc(OC)cc2)c2nc(N3CCN(C(=O)OCc4ccccc4)C[C@@H]3C)nc3c(I)cnn23)cc1]

8-iodo-2-(methanesulfonyl)-N,N-bis[(4-methoxyphenyl)methyl]pyrazolo[1,5-a][1,3,5]triazin-4-amine (Intermediate 127, 103 mg, 178 μmol) and benzyl (3S)-3-methylpiperazine-1-carboxylate (83.0 mg, 356 μmol; CAS-RN:[612493-87-5]) were provided in acetonitrile (2.6 mL), N,N-diisopropylethylamine (70 μL, 391 μmol; CAS-RN:[7087-68-5]) was added and the mixture was stirred at 60° C. for 1 h, for three days at 75° C. and for 1 day at 100° C. Benzyl (3S)-3-methylpiperazine-1-carboxylate (83.0 mg, 356 μmol; CAS-RN:[612493-87-5]) and N,N-diisopropylethylamine (70 μL, 391 μmol; CAS-RN:[7087-68-5]) were added and the mixture was stirred for 1 day at 100° C. In a second batch 8-iodo-2-(methanesulfonyl)-N,N-bis[(4-methoxyphenyl)methyl]pyrazolo[1,5-a][1,3,5]triazin-4-amine (Intermediate 127, 130 mg, 224 μmol) and benzyl (3S)-3-methylpiperazine-1-carboxylate (105 mg, 449 μmol; CAS-RN:[612493-87-5]) were provided in acetonitrile (3.3 mL), N,N-diisopropylethylamine (90 μL, 494 μmol; CAS-RN:[7087-68-5]) was added and the mixture was stirred at 150° C. for 1 h in a microwave. In a third batch 8-iodo-2-(methanesulfonyl)-N,N-bis[(4-methoxyphenyl)methyl]pyrazolo[1,5-a][1,3,5]triazin-4-amine (Intermediate 127, 107 mg, 185 μmol) and benzyl (3S)-3-methylpiperazine-1-carboxylate (87.0 mg, 369 μmol; CAS-RN:[612493-87-5]) were provided in acetonitrile (2.7 mL), N,N-diisopropylethylamine (70 μL, 406 μmol; CAS-RN:[7087-68-5]) was added and the mixture was stirred at 85° C. for 5 days. Benzyl (3S)-3-methylpiperazine-1-carboxylate (87.0 mg, 369 μmol; CAS-RN:[612493-87-5]) and N,N-diisopropylethylamine (70 μL, 406 μmol; CAS-RN:[7087-68-5]) were added and the mixture was stirred for 3 h at 85° C. Benzyl (3S)-3-methylpiperazine-1-carboxylate (43.5 mg, 185 μmol; CAS-RN:[612493-87-5]) was added and the mixture was stirred over night at 85° C. The three batches were combined, concentrated and purified by flash chromatography (silica gel, dichloromethane/ethyl acetate/ethanol gradient) to give 191 mg of a crude product. In a fourth batch 8-iodo-2-(methanesulfonyl)-N,N-bis[(4-methoxyphenyl)methyl]pyrazolo[1,5-a][1,3,5]triazin-4-amine (Intermediate 127, 1.00 g, 1.73 mmol) and benzyl (3S)-3-methylpiperazine-1-carboxylate (0.81 g, 3.45 mmol; CAS-RN:[612493-87-5]) were provided in acetonitrile (15 mL), N,N-diisopropylethylamine (660 μL, 3.80 mmol; CAS-RN:[7087-68-5]) was added and the mixture was stirred at 100° C. for 6 h, at rt over night, for two days at 100° C. and for three days at 80° C. The mixture was concentrated under reduced pressure and purified by flash chromatography (silica gel, dichloromethane/ethyl acetate/ethanol gradient) to give 322 mg of a crude product. The two crude products were combined and purified by flash chromatography twice (1. silica gel, dichloromethane/ethyl acetate/ethanol gradient, 2. silica gel, hexane/ethyl acetate gradient) to give 231 mg of the title compound.

LC-MS (Method 1): Rₜ=1.72 min; MS (ESIpos): m/z=734 [M+H]⁺

¹H-NMR (400 MHz, DMSO-d6) δ[ppm]: 1.007 (0.35), 1.172 (0.28), 1.232 (0.29), 1.988 (0.53), 2.332 (0.30), 2.518 (1.54), 2.523 (1.04), 3.116 (0.22), 3.719 (16.00), 3.807 (0.19), 3.840 (0.17), 5.104 (0.63), 5.117 (1.24), 5.148 (0.26), 6.868 (0.27), 6.875 (2.72), 6.880 (0.97), 6.892 (0.95), 6.897 (3.05), 6.904 (0.36), 7.216 (2.28), 7.238 (2.04), 7.299 (0.19), 7.307 (0.37), 7.311 (0.42), 7.321 (0.38), 7.324 (0.32), 7.333 (0.40), 7.343 (0.19), 7.362 (3.07), 7.374 (1.43), 7.952 (3.81), 8.010 (0.19).

### Intermediate 208

### benzyl (3S)-4-[4-{bis[(4-methoxyphenyl)methyl]amino}-8-(trifluoromethyl)pyrazolo[1,5-a][1,3,5]triazin-2-yl]-3-methylpiperazine-1-carboxylate

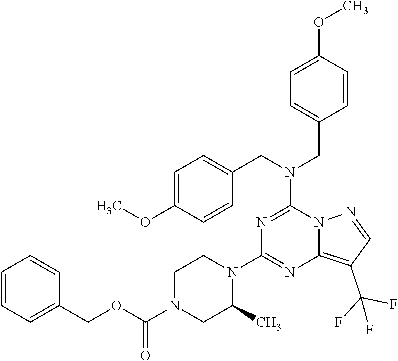 [SMILES: COc1ccc(CN(Cc2ccc(OC)cc2)c2nc(N3CCN(C(=O)OCc4ccccc4)C[C@@H]3C)nc3c(C(F)(F)F)cnn23)cc1]

To a suspension of benzyl (3S)-4-(4-{bis[(4-methoxyphenyl)methyl]amino}-8-iodopyrazolo[1,5-a][1,3,5]triazin-2-yl)-3-methylpiperazine-1-carboxylate (Intermediate 207, 866 mg, 1.18 mmol) and copper(I) iodide (899 mg, 4.72 mmol; CAS-RN:[7681-65-4]) in N,N-dimethylformamide (10 mL) was added methyl difluoro(fluorosulfonyl)acetate (600 μL, 4.72 mmol; CAS-RN:[680-16-9]) at rt under an argon atmosphere. The reaction mixture was stirred at 80° C. for 16 h. The reaction mixture was concentrated and purified by flash chromatography (silica gel, hexane/ethyl acetate gradient) to give 170 mg (21% yield) of the title compound.

LC-MS (Method 1): Rₜ=1.71 min; MS (ESIpos): m/z=676 [M+H]⁺

¹H-NMR (400 MHz, DMSO-d6) δ[ppm]: 0.852 (0.58), 0.870 (1.14), 0.888 (0.52), 1.232 (0.50), 2.012 (2.56), 2.518 (2.47), 2.523 (1.77), 3.372 (0.68), 3.724 (16.00), 5.102 (0.85), 5.115 (1.43), 6.882 (2.95), 6.904 (3.15), 7.223 (0.45), 7.240 (2.84), 7.261 (2.35), 7.310 (0.49), 7.320 (0.44), 7.331 (0.50), 7.360 (4.02), 7.372 (1.90), 8.010 (0.53), 8.210 (1.84).

### Intermediate 209

### N-[(4-methoxyphenyl)methyl]-2-[(2S)-2-methylpiperazin-1-yl]-8-(trifluoromethyl)pyrazolo[1,5-a][1,3,5]triazin-4-amine

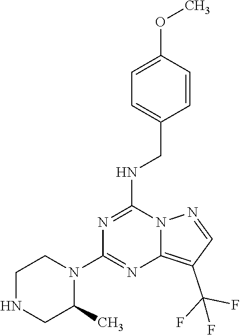 [SMILES: COc1ccc(CNc2nc(N3CCNC[C@@H]3C)nc3c(C(F)(F)F)cnn23)cc1]

A mixture of benzyl (3S)-4-[4-{bis[(4-methoxyphenyl)methyl]amino}-8-(trifluoromethyl)pyrazolo[1,5-a][1,3,5]triazin-2-yl]-3-methylpiperazine-1-carboxylate (Intermediate 208, 168 mg, 249 μmol) in trifluoroacetic acid (1.9 mL, 25.0 mmol; CAS-RN:[76-05-1]) was stirred at rt for 1 h, for 30 min at 60° C., for 2.5 h at 40° C. and 30 min at 60° C. The reaction mixture was concentrated and purified by flash chromatography (amino-phase silica; gradient dichloromethane/ethyl acetate) to give 76.0 mg of the title compound.

LC-MS (Method 2): Rₜ=1.26 min; MS (ESIpos): m/z=422 [M+H]⁺

### Intermediate 210

### benzyl (3S)-4-{4-[(4-methoxybenzyl)amino]-8-(trifluoromethyl)pyrazolo[1,5-a][1,3,5]triazin-2-yl}-3-methylpiperazine-1-carboxylate

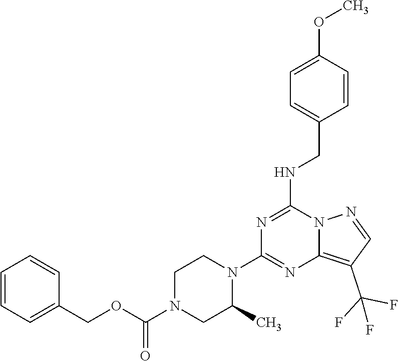 [SMILES: COc1ccc(CNc2nc(N3CCN(C(=O)OCc4ccccc4)C[C@@H]3C)nc3c(C(F)(F)F)cnn23)cc1]

The title compound (39.0 mg) was isolated as a side product in the synthesis of Intermediate 209.

LC-MS (Method 2): Rₜ=1.52 min; MS (ESIpos): m/z=556 [M+H]⁺

### Intermediate 211

### 2-[(2S)-2-methylpiperazin-1-yl]-8-(trifluoromethyl)pyrazolo[1,5-a][1,3,5]triazin-4-amine

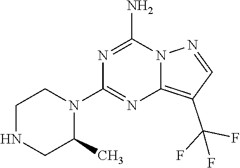 [SMILES: C[C@H]1CNCCN1c1nc(N)n2ncc(C(F)(F)F)c2n1]

The reaction was executed in two separate batches using two different starting materials. In the first batch a mixture of N-[(4-methoxyphenyl)methyl]-2-[(2S)-2-methylpiperazin-1-yl]-8-(trifluoromethyl)pyrazolo[1,5-a][1,3,5]triazin-4-amine (Intermediate 209, 75.0 mg) in trifluoroacetic acid (0.5 mL, 6.49 mmol; CAS-RN:[76-05-1]) was stirred at rt for 75 min. In the second batch a mixture of benzyl (3S)-4-{4-[(4-methoxybenzyl)amino]-8-(trifluoromethyl)pyrazolo[1,5-a][1,3,5]triazin-2-yl}-3-methylpiperazine-1-carboxylate (Intermediate 210, 35 mg) in trifluoroacetic acid (0.5 mL, 6.49 mmol; CAS-RN:[76-05-1]) was stirred at rt for 60 min. Both batches were combined, diluted with ethyl acetate and washed with aqueous saturated sodiumbicarbonate solution. The organic layer was separated and concentrated. The residue was purified by flash chromatography (amino-phase silica; gradient dichloromethane/ethyl actate) to give 43.0 mg of the title compound.

LC-MS (Method 2): Rₜ=0.98 min; MS (ESIpos): m/z=302 [M+H]⁺

### Intermediate 212

### benzyl (3S)-4-[4-amino-8-(trifluoromethyl)pyrazolo[1,5-a][1,3,5]triazin-2-yl]-3-methylpiperazine-1-carboxylate

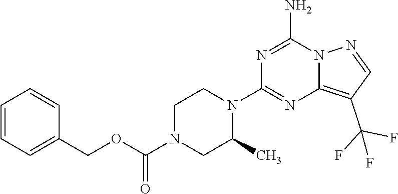 [SMILES: C[C@H]1CN(C(=O)OCc2ccccc2)CCN1c1nc(N)n2ncc(C(F)(F)F)c2n1]

To a mixture of 2-[(2S)-2-methylpiperazin-1-yl]-8-(trifluoromethyl)pyrazolo[1,5-a][1,3,5]triazin-4-amine (Intermediate 211, 42.0 mg) and sodium bicarbonate (35.1 mg, 418 μmol) in a mixture of tetrahydrofuran (1.0 mL) and water (1.0 mL) was added benzyl carbonochloridate (40 μL, 280 μmol; CAS-RN:[501-53-1]). The mixture was stirred for 2 h at rt, concentrated under reduced pressure and purified flash chromatography (amino-phase silica; gradient hexane/ethyl actate) to give 31.0 mg of the title compound.

LC-MS (Method 2): Rₜ=1.31 min; MS (ESIpos): m/z=436 [M+H]⁺

¹H-NMR (400 MHz, DMSO-d6) δ[ppm]: 0.852 (0.32), 1.102 (1.91), 1.118 (1.91), 1.155 (0.37), 1.173 (0.74), 1.190 (0.43), 1.233 (0.90), 1.988 (1.38), 2.005 (0.16), 2.327 (3.24), 2.332 (2.29), 2.336 (1.01), 2.518 (16.00), 2.523 (10.84), 2.669 (3.30), 2.673 (2.29), 2.678 (1.01), 3.871 (0.37), 3.903 (0.32), 4.017 (0.43), 4.036 (0.37), 4.427 (0.16), 4.874 (0.21), 5.088 (0.21), 5.121 (0.90), 5.135 (1.44), 5.166 (0.27), 7.318 (0.43), 7.331 (0.43), 7.340 (0.48), 7.372 (3.46), 7.384 (1.97), 7.956 (0.21), 8.175 (1.81), 8.551 (0.37).

### Intermediate 213

### benzyl (3S)-4-[4-{[(4-fluoro-1-{[2-(trimethylsilyl)ethoxy]methyl}-1H-benzimidazol-2-yl)methyl]amino}-8-(trifluoromethyl)pyrazolo[1,5-a][1,3,5]triazin-2-yl]-3-methylpiperazine-1-carboxylate

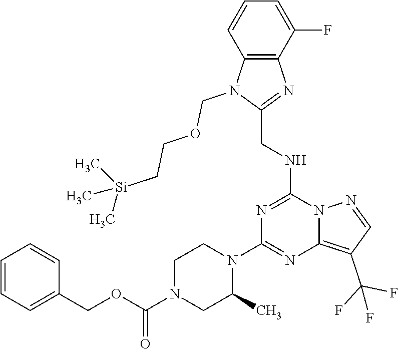 [SMILES: C[C@H]1CN(C(=O)OCc2ccccc2)CCN1c1nc(NCc2nc3c(F)cccc3n2COCC[Si](C)(C)C)n2ncc(C(F)(F)F)c2n1]

To a solution of benzyl (3S)-4-[4-amino-8-(trifluoromethyl)pyrazolo[1,5-a][1,3,5]triazin-2-yl]-3-methylpiperazine-1-carboxylate (Intermediate 212, 30.0 mg) in N,N-dimethylformamide (1.5 mL) was added sodium hydride (2.6 mg, 60% in mineral oil, 66 μmol; CAS-RN:[7646-69-7]) at room temperature. After stirring at 30° C. for 15 min, 2-(chloromethyl)-4-fluoro-1-{[2-(trimethylsilyl)ethoxy]methyl}-1H-benzimidazole (Intermediate 180, 26.0 mg, 82.7 μmol) was added. The reaction mixture was stirred for 3 h at room temperature followed by 16 h at 50° C. The reaction mixture was concentrated under reduced pressure. The residue was purified by flash chromatography (silica gel amino-phase, hexane/ethyl acetate gradient) to give 39.0 mg of the title compound.

LC-MS (Method 2): Rₜ=1.69 min; MS (ESIpos): m/z=714 [M+H]⁺

¹H-NMR (400 MHz, DMSO-d6) δ[ppm]: −0.190 (16.00), −0.181 (1.82), −0.169 (1.85), −0.155 (0.24), −0.091 (0.67), 0.482 (0.36), 0.499 (0.36), 0.627 (0.20), 0.852 (0.20), 1.035 (0.16), 1.085 (0.24), 1.102 (0.38), 1.118 (0.38), 1.233 (0.53), 1.259 (0.36), 2.332 (0.96), 2.336 (0.40), 2.518 (5.21), 2.523 (3.81), 2.673 (0.96), 2.678 (0.42), 2.996 (0.27), 3.361 (0.56), 3.378 (0.53), 3.385 (0.53), 3.403 (0.42), 3.702 (0.18), 4.968 (0.53), 5.079 (0.87), 5.092 (0.98), 5.121 (0.27), 5.135 (0.29), 5.638 (0.29), 5.666 (1.11), 5.681 (0.98), 5.710 (0.24), 7.005 (0.29), 7.027 (0.22), 7.199 (0.20), 7.219 (0.38), 7.231 (0.38), 7.251 (0.20), 7.347 (1.94), 7.358 (1.56), 7.373 (1.36), 7.467 (1.02), 7.487 (0.87), 8.019 (0.20), 8.174 (0.33), 8.240 (2.07), 9.363 (0.24).

### Intermediate 214

### 2-[(2S)-2,4-dimethylpiperazin-1-yl]-N-[(4-fluoro-1-{[2-(trimethylsilyl)ethoxy]methyl}-1H-benzimidazol-2-yl)methyl]-8-(trifluoromethyl)pyrazolo[1,5-a][1,3,5]triazin-4-amine

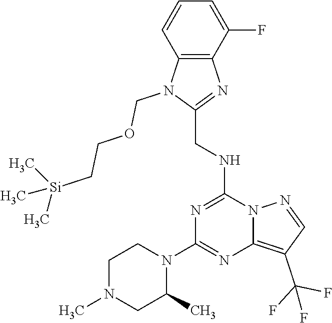 [SMILES: C[C@H]1CN(C)CCN1c1nc(NCc2nc3c(F)cccc3n2COCC[Si](C)(C)C)n2ncc(C(F)(F)F)c2n1]

To a solution of benzyl (3S)-4-[4-{[(4-fluoro-1-{[2-(trimethylsilyl)ethoxy]methyl}-1H-benzimidazol-2-yl)methyl]amino}-8-(trifluoromethyl)pyrazolo[1,5-a][1,3,5]triazin-2-yl]-3-methylpiperazine-1-carboxylate (Intermediate 213, 38.0 mg) in 13.0 mL of methanol under an argon atmosphere was added an aqueous formaldehyde solution (7.9 μL, 37% purity, 110 μmol) and palladium (146.0 mg, 10% on activated carbon). The argon atmosphere was replaced by hydrogen and the mixture was stirred for 4 hours at room temperature. The hydrogen atmosphere was replaced by air and the mixture was stirred over night at room temperature. The mixture was filtered and concentrated to give 30.0 mg of the title compound.

LC-MS (Method 2): Rₜ=1.62 min; MS (ESIpos): m/z=594 [M+H]⁺

¹H-NMR (400 MHz, DMSO-d6) δ[ppm]: −0.178 (16.00), −0.160 (0.94), −0.147 (1.31), −0.100 (0.45), 0.492 (0.27), 0.852 (0.31), 1.232 (1.08), 1.256 (0.41), 2.094 (0.78), 2.518 (8.38), 2.523 (6.11), 2.580 (0.31), 3.371 (0.51), 3.395 (0.37), 4.965 (0.53), 4.978 (0.47), 5.634 (0.25), 5.662 (0.76), 5.681 (0.76), 5.711 (0.27), 7.001 (0.29), 7.022 (0.39), 7.049 (0.33), 7.209 (0.20), 7.229 (0.37), 7.241 (0.39), 7.260 (0.18), 7.466 (0.84), 7.486 (0.72), 8.213 (0.41).

### Intermediate 215

### ethyl [(4-methyl-1H-pyrazol-5-yl)carbamothioyl]carbamate

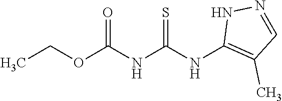

Ethyl carbonisothiocyanatidate (2.6 mL, 22 mmol; CAS 16182-04-0) was dissolved in ethyl acetate (20 mL) under an argon atmosphere. At 80° C. 4-methyl-1H-pyrazol-5-amine (2.00 g, 20.6 mmol) was added dropwise and the mixture was stirred for 1 h at 80° C. The mixture was cooled to 0° C., filtered, washed with ethyl acetate and the solid was dried under reduced pressure to give 3.77 g (80% yield) of the title compound.

LC-MS (Method 1): Rₜ=0.76 min; MS (ESIpos): m/z=229 [M+H]⁺

¹H-NMR (400 MHz, DMSO-d6) δ[ppm]: 1.134 (0.54), 1.234 (7.39), 1.241 (2.03), 1.252 (15.94), 1.258 (3.67), 1.270 (7.69), 1.276 (1.76), 1.899 (16.00), 1.987 (2.51), 2.518 (5.50), 2.522 (3.77), 4.178 (2.21), 4.196 (6.87), 4.213 (6.90), 4.227 (1.68), 4.231 (2.34), 4.245 (0.43), 7.504 (1.93), 8.493 (0.64), 10.997 (2.82), 11.232 (0.63), 11.339 (1.83), 11.627 (0.47), 12.468 (1.14).

### Intermediate 216

### 8-methyl-2-(methylsulfanyl)pyrazolo[1,5-a][1,3,5]triazin-4-ol

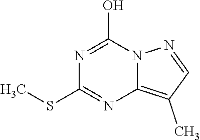

Ethyl [(4-methyl-1H-pyrazol-5-yl)carbamothioyl]carbamate (Intermediate 215, 6.01 g, 26.3 mmol) was stirred in sodium hydroxide (25.0 mL, 2.0 M, 50.0 mmol, CAS-RN:[1310-73-2]) for 4 h at rt. The mixture was diluted with ethanol (30 mL) and iodomethane (1.1 mL, 17 mmol; CAS-RN:[74-88-4]) was added. The mixture was stirred for 30 min at room temperature and cooled to 0° C. Water was added and the mixture was stirred for 15 min. The precipitate was isolated by filtration. The filtrate was concentrated to half of its original volume and the precipitate was isolated by filtration. The two precipitates were combined, suspended in water, acidified using aqueous sulfuric acid (2 mol/1), isolated by filtration and dried at 50° C. to give 2.17 g (42% yield) of the desired title compound.

LC-MS (Method 1): Rₜ=0.75 min; MS (ESIpos): m/z=197 [M+H]⁺

¹H-NMR (400 MHz, DMSO-d6) δ[ppm]: 2.090 (11.70), 2.518 (1.49), 2.523 (1.04), 2.555 (16.00), 7.864 (3.38), 12.717 (0.51).

### Intermediate 217

### 4-chloro-8-methyl-2-(methylsulfanyl)pyrazolo[1,5-a][1,3,5]triazine

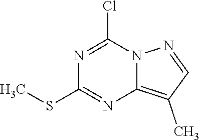

To a solution of 8-methyl-2-(methylsulfanyl)pyrazolo[1,5-a][1,3,5]triazin-4-ol (Intermediate 216, 2.17 g, 11.1 mmol) in toluene (50 mL) N,N-diisopropylethylamine (2.9 mL, 16.6 mmol; CAS-RN:[7087-68-5]) and phosphoric trichloride (5.7 mL, 60.8 mmol; CAS-RN:[10025-87-3]) were added. The mixture was stirred for 4 h under reflux. The mixture was concentrated and the residue was purified by flash chromatography (silica gel, dichloromethane/ethanol gradient) to give 2.08 g (87% yield) of the title compound.

LC-MS (Method 1): Rₜ=1.09 min; MS (ESIpos): m/z=215 [M+H]⁺

¹H-NMR (400 MHz, CHLOROFORM-d) 5 [ppm]: 2.290 (10.44), 2.627 (16.00), 8.019 (2.19).

### Intermediate 218

### N-[(4,5-difluoro-1H-benzimidazol-2-yl)methyl]-8-methyl-2-(methylsulfanyl)pyrazolo[1,5-a][1,3,5]triazin-4-amine

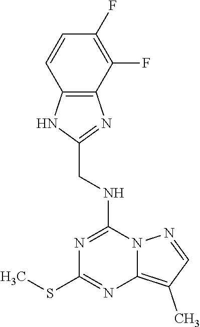 [SMILES: CSc1nc(NCc2nc3c(F)c(F)ccc3[nH]2)n2ncc(C)c2n1]

To a solution of 4-chloro-8-methyl-2-(methylsulfanyl)pyrazolo[1,5-a][1,3,5]triazine (Intermediate 217, 500 mg, 2.33 mmol) in dichloromethane (10 mL) 1-(4,5-difluoro-1H-benzimidazol-2-yl)methanamine hydrogen chloride (1/2) (626 mg, 2.45 mmol) and N,N-diisopropylethylamine (1.4 mL, 8.15 mmol; CAS-RN:[7087-68-5]) were added. The mixture was stirred over night at room temperature. The mixture was concentrated and water was added. The precipitated was isolated by filtration and dried at 50° C. to give 805 mg (89% yield) of the title compound.

LC-MS (Method 1): Rₜ=1.11 min; MS (ESIpos): m/z=362 [M+H]⁺

¹H-NMR (400 MHz, DMSO-d6) δ[ppm]: 1.035 (0.57), 1.052 (1.12), 1.070 (0.62), 2.114 (0.82), 2.149 (13.36), 2.223 (0.48), 2.332 (0.57), 2.376 (0.61), 2.406 (16.00), 2.518 (3.37), 2.523 (2.28), 2.595 (0.62), 2.673 (0.60), 4.908 (2.81), 4.923 (2.79), 7.188 (0.82), 7.204 (1.02), 7.212 (2.14), 7.226 (2.48), 7.234 (0.61), 7.992 (0.82), 8.002 (3.80), 9.300 (0.55), 9.316 (1.19), 9.331 (0.53), 12.713 (1.02).

### Intermediate 219

### N-[(4,5-difluoro-1H-benzimidazol-2-yl)methyl]-2-(methanesulfonyl)-8-methylpyrazolo[1,5-a][1,3,5]triazin-4-amine

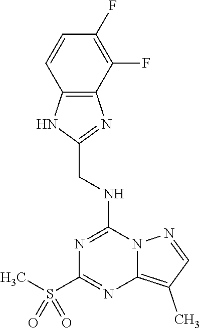 [SMILES: Cc1cnn2c(NCc3nc4c(F)c(F)ccc4[nH]3)nc(S(C)(=O)=O)nc12]

To a solution of N-[(4,5-difluoro-1H-benzimidazol-2-yl)methyl]-8-methyl-2-(methylsulfanyl)pyrazolo[1,5-a][1,3,5]triazin-4-amine (Intermediate 218, 800 mg, 2.21 mmol) in dichloromethane (20 mL) at 0° C. was added 3-chlorobenzene-1-carboperoxoic acid (1.49 g, 77% purity, 6.64 mmol; CAS-RN:[937-14-4]) and the mixture was stirred for two hours. An aqueous saturated sodiumcarbonate solution was added till a basic pH was reached and the mixture was stirred for 30 min at room temperature. The layers were separated and the aqueous layer was extracted with dichloromethane. The combined organic layers were filtered through a hydrophobic filter and concentrated to give 850 mg (96% yield) of the title compound.

LC-MS (Method 1): Rₜ=0.92 min; MS (ESIpos): m/z=394 [M+H]⁺

¹H-NMR (400 MHz, DMSO-d6) δ[ppm]: 1.180 (0.44), 1.234 (0.46), 1.351 (5.56), 1.739 (0.72), 1.747 (0.69), 1.757 (2.17), 1.765 (0.74), 1.773 (0.82), 2.180 (0.75), 2.263 (13.89), 2.287 (0.43), 2.518 (8.46), 2.522 (5.95), 3.230 (16.00), 3.251 (0.53), 3.303 (0.61), 3.316 (1.50), 3.582 (0.81), 3.592 (0.74), 3.598 (1.91), 3.604 (0.67), 3.608 (0.52), 3.615 (0.81), 5.033 (3.12), 5.048 (3.13), 5.758 (0.84), 6.867 (0.42), 7.198 (0.82), 7.214 (1.14), 7.219 (2.14), 7.225 (1.28), 7.235 (2.26), 7.526 (0.92), 7.546 (2.08), 7.566 (1.38), 7.693 (0.71), 7.697 (0.75), 7.699 (0.98), 7.702 (0.83), 7.714 (0.55), 7.716 (0.57), 7.719 (0.76), 7.722 (0.60), 7.881 (0.53), 7.885 (1.16), 7.887 (1.00), 7.891 (1.40), 7.896 (1.56), 7.901 (2.16), 7.904 (1.22), 7.908 (0.54), 8.285 (3.79), 10.005 (0.53), 10.020 (1.10), 10.034 (0.56), 12.742 (1.15).

### Intermediate 220

### N-[(1H-benzimidazol-2-yl)methyl]-8-methyl-2-(methylsulfanyl)pyrazolo[1,5-a][1,3,5]triazin-4-amine

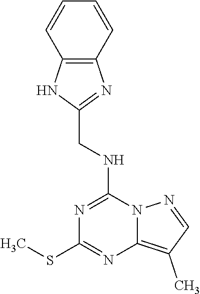 [SMILES: CSc1nc(NCc2nc3ccccc3[nH]2)n2ncc(C)c2n1]

To a solution of 4-chloro-8-methyl-2-(methylsulfanyl)pyrazolo[1,5-a][1,3,5]triazine (Intermediate 217, 500 mg, 2.33 mmol) in dichloromethane (15 mL) 1-(1H-benzimidazol-2-yl)methanamine (360 mg, 2.45 mmol) and N,N-diisopropylethylamine (810 μL, 4.7 mmol; CAS-RN:[7087-68-5]) were added. The mixture was stirred over night at room temperature. 1-(1H-benzimidazol-2-yl)methanamine (40 mg, 0.27 mmol) was added and the mixture was stirred for 4 h at room temperature. 1-(1H-benzimidazol-2-yl)methanamine (40 mg, 0.27 mmol) was added and the mixture was stirred for 4 h at room temperature. The mixture was concentrated and water was added. The precipitate was isolated by filtration and dried under reduced pressure at 50° C. to give 662 mg (83% yield) of the title compound.

LC-MS (Method 1): Rₜ=0.83 min; MS (ESIpos): m/z=326 [M+H]⁺

¹H-NMR (400 MHz, DMSO-d6) δ[ppm]: 1.172 (0.26), 1.238 (0.19), 1.253 (0.19), 1.265 (0.18), 1.988 (0.45), 2.069 (0.25), 2.152 (11.17), 2.238 (0.19), 2.332 (0.23), 2.417 (16.00), 2.518 (1.51), 2.523 (0.99), 2.556 (0.23), 2.609 (0.18), 2.673 (0.25), 4.903 (2.71), 4.919 (2.71), 5.759 (0.25), 7.125 (1.10), 7.132 (1.19), 7.140 (1.24), 7.147 (1.21), 7.427 (0.25), 7.525 (0.26), 7.994 (3.82), 9.238 (0.54), 9.253 (1.18), 9.268 (0.53), 12.293 (0.58).

### Intermediate 221

### N-[(1H-benzimidazol-2-yl)methyl]-2-(methanesulfonyl)-8-methylpyrazolo[1,5-a][1,3,5]triazin-4-amine

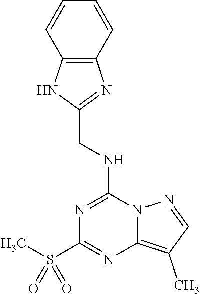 [SMILES: Cc1cnn2c(NCc3nc4ccccc4[nH]3)nc(S(C)(=O)=O)nc12]

To a solution of N-[(1H-benzimidazol-2-yl)methyl]-8-methyl-2-(methylsulfanyl)pyrazolo[1,5-a][1,3,5]triazin-4-amine (Intermediate 220, 660 mg, 2.03 mmol) in dichloromethane (20 mL) at 0° C. was added 3-chlorobenzene-1-carboperoxoic acid acid (1.36 g, 77% purity, 6.08 mmol; CAS-RN:[937-14-4]) and the mixture was stirred for two hours. An aqueous saturated sodiumbicarbonate solution was added till a basic pH was reached and the mixture was stirred for 30 min at room temperature. The layers were separated and the aqueous layer was extracted with dichloromethane. The combined organic layers were filtered through a hydrophobic filter and concentrated to give 760 mg of the title compound.

LC-MS (Method 1): Rₜ=0.67 min; MS (ESIpos): m/z=358 [M+H]⁺

¹H-NMR (400 MHz, DMSO-d6) δ[ppm]: 2.084 (0.49), 2.215 (0.26), 2.266 (9.68), 2.287 (0.35), 2.518 (5.65), 2.523 (3.95), 2.722 (0.45), 3.206 (0.23), 3.236 (16.00), 3.250 (0.67), 3.283 (0.35), 3.373 (0.41), 5.026 (2.25), 5.041 (2.29), 5.759 (6.14), 7.134 (1.29), 7.142 (1.37), 7.150 (1.45), 7.157 (1.42), 7.528 (0.74), 7.547 (1.23), 7.568 (0.78), 7.695 (0.38), 7.698 (0.42), 7.701 (0.41), 7.704 (0.41), 7.716 (0.28), 7.718 (0.31), 7.721 (0.36), 7.724 (0.30), 7.885 (0.56), 7.889 (0.51), 7.892 (0.67), 7.897 (0.77), 7.902 (1.08), 7.905 (0.59), 8.279 (3.56), 9.961 (0.46), 9.976 (0.96), 9.991 (0.45), 12.309 (0.35).

### Intermediate 222

### tert-butyl 3-(4-{[(4,5-difluoro-1H-benzimidazol-2-yl)methyl]amino}-8-methylpyrazolo[1,5-a][1,3,5]triazin-2-yl)-3,8-diazabicyclo[3.2.1]octane-8-carboxylate

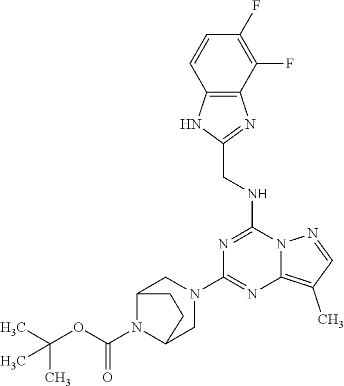 [SMILES: Cc1cnn2c(NCc3nc4c(F)c(F)ccc4[nH]3)nc(N3CC4CCC(C3)N4C(=O)OC(C)(C)C)nc12]

N-[(4,5-difluoro-1H-benzimidazol-2-yl)methyl]-2-(methanesulfonyl)-8-methylpyrazolo[1,5-a][1,3,5]triazin-4-amine (Intermediate 219, 150 mg, 381 μmol) and tert-butyl 3,8-diazabicyclo[3.2.1]octane-8-carboxylate (121 mg, 572 μmol) were provided in acetonitrile (3.2 mL), N,N-diisopropylethylamine (100 μL, 570 μmol; CAS-RN:[7087-68-5]) was added and the mixture was stirred at 70° C. over night. The mixture was concentrated under reduced pressure. The residue was purified by HPLC (Method HT acidic) to give 62.0 mg (29% yield) of the title compound.

LC-MS (Method 2): Rₜ=1.31 min; MS (ESIpos): m/z=526 [M+H]⁺

¹H-NMR (400 MHz, DMSO-d6) δ[ppm]: 1.385 (16.00), 1.416 (0.88), 1.437 (0.37), 2.012 (6.05), 2.539 (0.29), 2.834 (0.24), 4.091 (0.17), 4.799 (1.01), 4.812 (0.94), 7.167 (0.33), 7.183 (0.48), 7.193 (0.72), 7.206 (0.77), 7.770 (1.99), 8.966 (0.42), 12.676 (0.39).

### Intermediate 223

### tert-butyl 3-(4-{[(1H-benzimidazol-2-yl)methyl]amino}-8-methylpyrazolo[1,5-a][1,3,5]triazin-2-yl)-3,8-diazabicyclo[3.2.1]octane-8-carboxylate

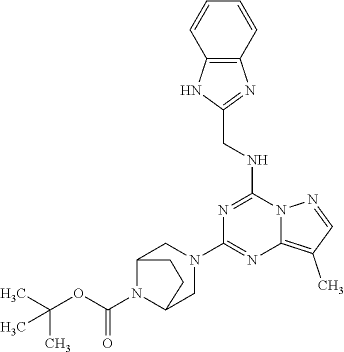 [SMILES: Cc1cnn2c(NCc3nc4ccccc4[nH]3)nc(N3CC4CCC(C3)N4C(=O)OC(C)(C)C)nc12]

N-[(1H-benzimidazol-2-yl)methyl]-2-(methanesulfonyl)-8-methylpyrazolo[1,5-a][1,3,5]triazin-4-amine (Intermediate 221, 150 mg, 420 μmol) and tert-butyl 3,8-diazabicyclo[3.2.1]octane-8-carboxylate (134 mg, 630 μmol) were provided in acetonitrile (3.2 mL), N,N-diisopropylethylamine (110 μL, 630 μmol; CAS-RN:[7087-68-5]) was added and the mixture was stirred at 70° C. over night. The mixture was concentrated under reduced pressure. The residue was purified by HPLC (Method HT acidic)_to give 65.0 mg (31% yield) of the title compound.

LC-MS (Method 2): Rₜ=1.27 min; MS (ESIneg): m/z=488 [M−H]⁻

¹H-NMR (400 MHz, DMSO-d6) δ[ppm]: 1.387 (16.00), 1.410 (0.43), 1.414 (0.63), 2.017 (5.59), 2.074 (0.20), 2.518 (0.99), 2.523 (0.68), 2.539 (0.39), 2.839 (0.23), 2.872 (0.30), 4.806 (0.94), 4.820 (0.91), 7.109 (0.66), 7.116 (0.69), 7.124 (0.72), 7.131 (0.72), 7.521 (0.67), 7.533 (0.19), 7.542 (1.42), 7.548 (0.17), 7.556 (0.16), 7.562 (0.86), 7.687 (0.46), 7.690 (0.53), 7.693 (0.53), 7.695 (0.50), 7.707 (0.39), 7.710 (0.38), 7.712 (0.40), 7.715 (0.38), 7.764 (2.26), 7.879 (0.36), 7.883 (0.77), 7.886 (0.69), 7.890 (0.91), 7.894 (1.18), 7.900 (1.57), 7.902 (0.88), 7.906 (0.22), 8.139 (0.70), 8.874 (0.27), 8.889 (0.56), 8.903 (0.26).

### Intermediate 224

### 8-iodo-2,4-bis(methylsulfanyl)pyrazolo[1,5-a][1,3,5]triazine

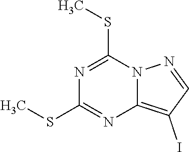

To a solution of 4-chloro-8-iodo-2-(methylsulfanyl)pyrazolo[1,5-a][1,3,5]triazine (Intermediate 125, 9.98 g, 30.6 mmol) in tetrahydrofuran (200 mL) was added sodium methanethiolate (2.62 g, 90% purity, 33.6 mmol; CAS-RN:[5188-07-8]). The mixture was stirred for 16 h at room temperature. Sodium methanethiolate (952 mg, 90% purity, 12.2 mmol; CAS-RN:[5188-07-8]) was added and the mixture was stirred for 3 days at room temperature. The mixture was concentrated and water was added. The precipitate was isolated by give 7.01 g (66% yield) of the title compound.

LC-MS (Method 1): Rₜ=1.33 min; MS (ESIpos): m/z=339 [M+H]⁺

¹H-NMR (400 MHz, DMSO-d6) δ[ppm]: 2.518 (1.43), 2.523 (0.97), 2.603 (16.00), 2.668 (15.72), 8.307 (6.49).

### Intermediate 225

### 2,4-bis(methylsulfanyl)-8-(trifluoromethyl)pyrazolo[1,5-a][1,3,5]triazine

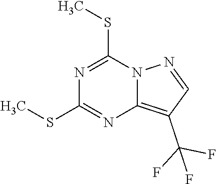 [SMILES: CSc1nc(SC)n2ncc(C(F)(F)F)c2n1]

To a suspension of 8-iodo-2,4-bis(methylsulfanyl)pyrazolo[1,5-a][1,3,5]triazine (Intermediate 224, 4.65 g, 13.8 mmol) and copper(I) iodide (10.6 g, 55.0 mmol; CAS-RN:[7681-65-4]) in N,N-dimethylformamide (87 mL) was added methyl difluoro(fluorosulfonyl)acetate (7.0 mL, 55.0 mmol; CAS-RN:[680-15-9]) at rt under an argon atmosphere. The reaction mixture was stirred at 80° C. for 20 h. Methyl difluoro(fluorosulfonyl)acetate (3.5 mL, 22.5 mmol; CAS-RN:[680-15-9]) was added and the mixture was stirred for 24 h at 80° C. and 3 days at room temperature. The reaction mixture was concentrated and the residue was purified by column chromatography (silica gel, hexane/dichloromethane/ethyl acetate gradient) to give 2.85 g (72% yield) of the title compound.

LC-MS (Method 2): Rₜ=1.35 min; MS (ESIpos): m/z=281 [M+H]⁺

¹H-NMR (400 MHz, DMSO-d6) δ[ppm]: 2.518 (0.74), 2.523 (0.50), 2.603 (16.00), 2.693 (15.54), 8.622 (2.65).

### Intermediate 226

### N-[(4,5-difluoro-1H-benzimidazol-2-yl)methyl]-2-(methylsulfanyl)-8-(trifluoromethyl)pyrazolo[1,5-a][1,3,5]triazin-4-amine

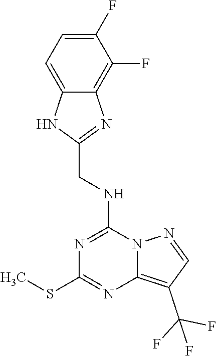 [SMILES: CSc1nc(NCc2nc3c(F)c(F)ccc3[nH]2)n2ncc(C(F)(F)F)c2n1]

To a solution of 2,4-bis(methylsulfanyl)-8-(trifluoromethyl)pyrazolo[1,5-a][1,3,5]triazine (Intermediate 225, 300 mg, 1.07 mmol) and 1-(4,5-difluoro-1H-benzimidazol-2-yl)methanamine hydrogen chloride (1/2) (384 mg, 1.50 mmol) in dichloromethane (9.0 mL) was added N,N-diisopropylethylamine (1.1 mL, 6.42 mmol; CAS-RN:[7087-68-5]). The mixture was stirred for 3.5 h in the microwave at 150° C. The reaction mixture was concentrated and purified by column chromatography (silica gel, amino-phase, dichloromethane/ethanol gradient) to give 370 mg (82% yield) of the title compound.

LC-MS (Method 1): Rₜ=1.22 min; MS (ESIpos): m/z=416 [M+H]⁺

¹H-NMR (400 MHz, DMSO-d6) δ[ppm]: 0.902 (0.71), 0.920 (0.47), 0.926 (0.78), 0.930 (0.57), 0.941 (5.39), 0.957 (5.27), 1.053 (0.46), 1.057 (0.53), 1.070 (0.46), 1.073 (0.53), 1.988 (0.57), 2.066 (0.86), 2.332 (0.77), 2.403 (0.63), 2.415 (16.00), 2.437 (0.42), 2.518 (3.93), 2.523 (2.83), 2.673 (0.77), 4.957 (5.79), 5.759 (0.89), 7.194 (0.74), 7.200 (0.45), 7.212 (0.80), 7.223 (0.83), 7.240 (1.01), 8.536 (3.53).

### Intermediate 227

### N-[(4,5-difluoro-1H-benzimidazol-2-yl)methyl]-2-(methanesulfonyl)-8-(trifluoromethyl)pyrazolo[1,5-a][1,3,5]triazin-4-amine

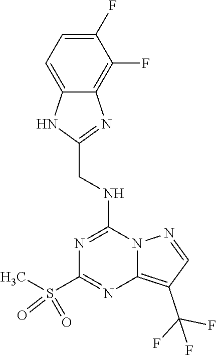 [SMILES: CS(=O)(=O)c1nc(NCc2nc3c(F)c(F)ccc3[nH]2)n2ncc(C(F)(F)F)c2n1]

To a solution of N-[(4,5-difluoro-1H-benzimidazol-2-yl)methyl]-2-(methylsulfanyl)-8-(trifluoromethyl)pyrazolo[1,5-a][1,3,5]triazin-4-amine (Intermediate 226, 368 mg, 886 μmol) in dichloromethane (21 mL) at 0° C. was added 3-chlorobenzene-1-carboperoxoic acid (612 mg, 3.54 mmol; CAS-RN:[937-14-4]) and the mixture was stirred for 17 hours at room temperature. 3-chlorobenzene-1-carboperoxoic acid (306 mg, 1.77 mmol; CAS-RN:[937-14-4]) was added and the mixture was stirred for 7 h at room temperature. The mixture was diluted with ethyl acetate, washed with an aqueous saturated sodium thiosulfate solution, filtered through a hydrophobic filter and concentrated to give 359 mg (91% yield) of the title compound.

LC-MS (Method 1): Rₜ=1.04 min; MS (ESIpos): m/z=448 [M+H]⁺

¹H-NMR (400 MHz, DMSO-d6) δ[ppm]: 0.902 (0.53), 1.107 (0.66), 1.172 (0.46), 1.190 (0.42), 1.218 (0.56), 1.234 (1.34), 1.263 (0.81), 1.281 (1.53), 1.299 (0.74), 1.352 (2.33), 1.367 (3.52), 1.382 (2.33), 1.445 (0.88), 1.461 (0.92), 1.988 (0.51), 2.066 (0.61), 2.084 (0.47), 2.287 (0.89), 2.332 (1.50), 2.336 (0.64), 2.518 (7.71), 2.523 (5.67), 2.673 (1.49), 2.678 (0.62), 2.767 (0.44), 3.278 (16.00), 3.299 (0.89), 3.599 (0.48), 3.618 (0.48), 3.638 (0.70), 5.081 (6.65), 5.759 (10.57), 7.207 (0.81), 7.224 (1.24), 7.234 (1.57), 7.246 (1.65), 7.533 (0.56), 7.876 (0.48), 7.885 (0.54), 7.889 (0.78), 7.894 (0.91), 8.829 (2.12), 12.701 (0.63).

### Intermediate 228

### N-[(4-fluoro-1H-benzimidazol-2-yl)methyl]-2-(methylsulfanyl)-8-(trifluoromethyl)pyrazolo[1,5-a][1,3,5]triazin-4-amine

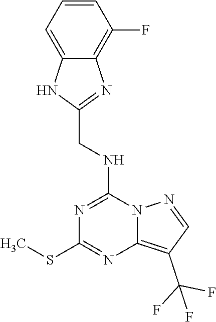 [SMILES: CSc1nc(NCc2nc3c(F)cccc3[nH]2)n2ncc(C(F)(F)F)c2n1]

To a solution of 2,4-bis(methylsulfanyl)-8-(trifluoromethyl)pyrazolo[1,5-a][1,3,5]triazine (Intermediate 225, 175 mg, 624 μmol) and 1-(4-fluoro-1H-benzimidazol-2-yl)methanamine hydrogen chloride (1/2) (178 mg, 749 μmol; CAS-RN:[2089257-74-7]) in acetonitrile (5.0 mL) was added N,N-diisopropylethylamine (650 μL, 3.75 mmol; CAS-RN:[7087-68-5]). The mixture was stirred for 4 h under reflux. Water was added and the mixture was extracted with ethyl acetate. The combined organic layers were filtered through a hydrophobic filter and concentrated. The residue was purified by column chromatography (silica gel, dichloromethane/ethanol gradient) to give 218 mg (84% yield) of the title compound.

LC-MS (Method 2): Rₜ=1.17 min; MS (ESIpos): m/z=398 [M+H]⁺

¹H-NMR (400 MHz, DMSO-d6) δ[ppm]: 1.052 (0.18), 2.408 (0.58), 2.420 (16.00), 2.436 (0.26), 2.518 (1.01), 2.523 (0.65), 4.967 (5.49), 5.758 (3.46), 6.947 (0.30), 6.968 (0.47), 6.992 (0.36), 7.113 (0.62), 7.125 (0.67), 7.134 (1.22), 7.146 (1.28), 7.153 (0.75), 7.166 (0.67), 7.268 (0.31), 8.541 (3.84).

### Intermediate 229

### N-[(4-fluoro-1H-benzimidazol-2-yl)methyl]-2-(methanesulfonyl)-8-(trifluoromethyl)pyrazolo[1,5-a][1,3,5]triazin-4-amine

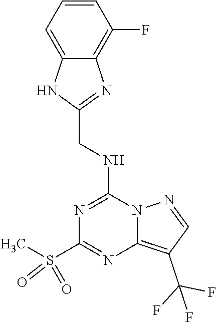 [SMILES: CS(=O)(=O)c1nc(NCc2nc3c(F)cccc3[nH]2)n2ncc(C(F)(F)F)c2n1]

To a solution of N-[(4-fluoro-1H-benzimidazol-2-yl)methyl]-2-(methylsulfanyl)-8-(trifluoromethyl)pyrazolo[1,5-a][1,3,5]triazin-4-amine (Intermediate 228, 105 mg, 264 μmol) in dichloromethane (6.3 mL) 3-chlorobenzene-1-carboperoxoic acid (182 mg, 1.06 mmol; CAS-RN:[937-14-4]) was added and the mixture was stirred for 17 hours at room temperature. 3-chlorobenzene-1-carboperoxoic acid (91.0 mg, 0.53 mmol; CAS-RN:[937-14-4]) was added and the mixture was stirred for 7 h at room temperature. The mixture was diluted with ethyl acetate, washed with an aqueous saturated sodium thiosulfate solution, filtered through a hydrophobic filter and concentrated. The residue was purified by column chromatography (silica gel, dichloromethane/ethyl acetate gradient) to give 59 mg (46% yield) of the title compound.

LC-MS (Method 1): Rₜ=0.99 min; MS (ESIpos): m/z=430 [M+H]⁺

¹H-NMR (400 MHz, DMSO-d6) δ[ppm]: 0.852 (0.21), 1.154 (3.61), 1.172 (7.00), 1.191 (3.39), 1.232 (0.79), 1.907 (0.17), 1.988 (13.90), 2.327 (1.48), 2.331 (1.03), 2.336 (0.43), 2.518 (5.06), 2.523 (3.27), 2.669 (1.46), 2.674 (1.00), 2.678 (0.45), 3.269 (0.33), 3.287 (16.00), 3.648 (0.43), 4.000 (0.91), 4.018 (2.82), 4.035 (2.87), 4.053 (0.93), 5.092 (3.65), 5.760 (0.43), 6.944 (0.67), 6.964 (0.86), 6.972 (0.72), 6.992 (0.81), 7.036 (0.21), 7.062 (0.21), 7.128 (0.62), 7.140 (0.67), 7.148 (1.19), 7.160 (1.10), 7.168 (0.69), 7.180 (0.60), 7.253 (1.79), 7.273 (1.29), 7.416 (0.31), 7.436 (0.26), 8.827 (0.19), 8.850 (3.61), 10.539 (0.64), 12.586 (1.03), 12.813 (0.31).

### Intermediate 230

### tert-butyl 4-[(4-{[(1H-benzimidazol-2-yl)methyl]amino}-8-bromopyrazolo[1,5-a][1,3,5]triazin-2-yl)oxy]piperidine-1-carboxylate

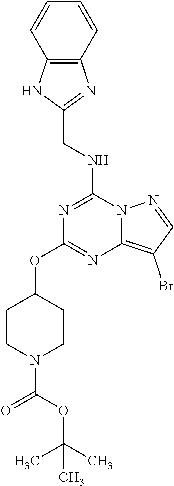 [SMILES: CC(C)(C)OC(=O)N1CCC(Oc2nc(NCc3nc4ccccc4[nH]3)n3ncc(Br)c3n2)CC1]

tert-butyl 4-hydroxypiperidine-1-carboxylate (251 mg, 1.25 mmol) was dissolved in tetrahydrofuran (4.0 mL) at 0° C. Sodiumhydride (30.7 mg, 60% in mineral oil, 0.77 mmol; CAS-RN:[7646-69-7]) was added and the mixture was stirred for 25 min. N-[(1H-benzimidazol-2-yl)methyl]-8-bromo-2-(methanesulfonyl)pyrazolo[1,5-a][1,3,5]triazin-4-amine (Intermediate 9, 150 mg, 320 μmol) was added and the mixture was stirred for 3 days at room temperature. Water was added and the mixture was extracted with ethyl acetate. The combined organic layers were concentrated and the residue was purified by column chromatography (silica gel, dichloromethane/ethanol gradient) to give 218 mg of the title compound.

LC-MS (Method 2): Rₜ=1.30 min; MS (ESIpos): m/z=543 [M+H]⁺

### Intermediate 231

### N,N-bis[(4-methoxyphenyl)methyl]-2-(morpholin-4-yl)-8-(1,3-thiazol-2-yl)pyrazolo[1,5-a][1,3,5]triazin-4-amine

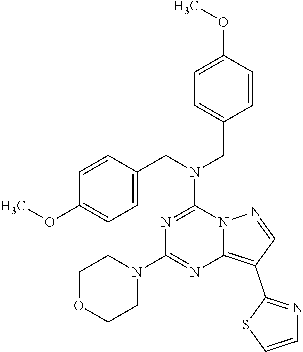 [SMILES: COc1ccc(CN(Cc2ccc(OC)cc2)c2nc(N3CCOCC3)nc3c(-c4nccs4)cnn23)cc1]

To a solution of 1,3-thiazole (813 mg, 9.55 mmol) in tetrahydrofuran (40 mL) was added n-butyllithium (3.8 mL, 9.5 mmol, 2.5 M in hexane) at −65° C. under nitrogen atmosphere. After stirring at −65° C. for 10 minutes under nitrogen atmosphere, zinc dichloride (2.79 g, 20.5 mmol) was added to the reaction mixture at −65° C.˜−60° C. under nitrogen atmosphere. The reaction mixture was stirred until it was warmed to room temperature. 8-iodo-N,N-bis[(4-methoxyphenyl)methyl]-2-(morpholin-4-yl)pyrazolo[1,5-a][1,3,5]triazin-4-amine (Intermediate 128, 4.00 g, 6.82 mmol) and tetrakis(triphenylphosphine)palladium(0) (394 mg, 0.341 mmol) were added to the above mixture at room temperature. After stirring at 60° C. for 16 hours under nitrogen atmosphere, the reaction mixture was quenched with water and extracted with ethyl acetate. The organic phase was washed with water and brine, dried over anhydrous sodium sulfate and concentrated to give a residue. The residue was purified by flash chromatography (petroleum ether: ethyl acetate=20:1 to 5:1) to give N,N-bis(4-methoxybenzyl)-2-(morpholin-4-yl)-8-(1,3-thiazol-2-yl)pyrazolo[1,5-a][1,3,5]triazin-4-amine (3.30 g, 89% yield) as a yellow solid.

¹H-NMR (400 MHz, DMSO-d₆): 5 [ppm]=8.41 (s, 1H), 7.75 (d, J=3.2 Hz, 1H), 7.56 (d, J=3.2 Hz, 1H), 7.27 (d, J=8.4 Hz, 4H), 6.91 (d, J=8.4 Hz, 4H), 5.12 (s, 4H), 3.95-3.76 (m, 4H), 3.74 (s, 6H), 3.71-3.58 (m, 4H).

### Intermediate 232

### 2-(morpholin-4-yl)-8-(1,3-thiazol-2-yl)pyrazolo[1,5-a][1,3,5]triazin-4-amine

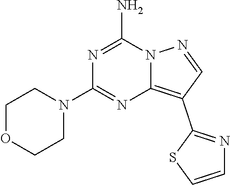 [SMILES: Nc1nc(N2CCOCC2)nc2c(-c3nccs3)cnn12]

A solution of N,N-bis[(4-methoxyphenyl)methyl]-2-(morpholin-4-yl)-8-(1,3-thiazol-2-yl)pyrazolo[1,5-a][1,3,5]triazin-4-amine (Intermediate 231, 3.30 g, 6.07 mmol) in trifluoroacetic acid (20 mL) was stirred at 80° C. for 3 hours. After cooled to room temperature, the reaction mixture was poured into water. After adjusted to pH=7 by saturated sodium bicarbonate aqueous solution, the mixture was filtered and the filter cake was washed with water and dried under vacuum to give 2-(morpholin-4-yl)-8-(1,3-thiazol-2-yl)pyrazolo[1,5-a][1,3,5]triazin-4-amine (1.36 g, 74% yield) as a white solid.

LC-MS (Method D): Rₜ=0.62 min; MS (ESIpos): m/z=304.1 [M+H]⁺.

### Intermediate 233

### N-[(4,5-difluoro-1-{[2-(trimethylsilyl)ethoxy]methyl}-1H-benzimidazol-2-yl)methyl]-2-(morpholin-4-yl)-8-(1,3-thiazol-2-yl)pyrazolo[1,5-a][1,3,5]triazin-4-amine

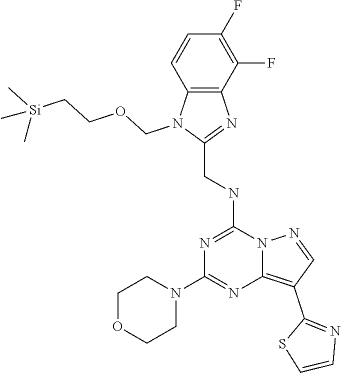 [SMILES: C[Si](C)(C)CCOCn1c(CNc2nc(N3CCOCC3)nc3c(-c4nccs4)cnn23)nc2c(F)c(F)ccc21]

The reaction was performed in two separate batches. To a solution of 2-(morpholin-4-yl)-8-(1,3-thiazol-2-yl)pyrazolo[1,5-a][1,3,5]triazin-4-amine (Intermediate 232, 100/200 mg, 0.33/0.659 mmol) in N,N-dimethylformamide (2.0/4.0 mL) was added sodium hydride (41.1/42.2 mg, 0.502/1.05 mmol, 60% purity in mineral oil) at room temperature. After stirring at 60° C. for 15 minutes, 2-(chloromethyl)-4,5-difluoro-1-{[2-(trimethylsilyl)ethoxy]methyl}-1H-benzimidazole (Intermediate 158, 108/219 mg, 0.33/0.659 mmol) was added in one portion. The reaction mixture was stirred at room temperature for 16 hours. The mixtures were combined, quenched with water and extracted with ethyl acetate. The organic phase was washed with water and brine, dried over anhydrous sodium sulfate, and concentrated to give N-[(4,5-difluoro-1-{[2-(trimethylsilyl)ethoxy]methyl}-1H-benzimidazol-2-yl)methyl]-2-(morpholin-4-yl)-8-(1,3-thiazol-2-yl)pyrazolo[1,5-a][1,3,5]triazin-4-amine (750 mg, 71% purity) as yellow oil.

LC-MS (Method D): Rₜ=0.889 min; MS (ESIpos): m/z=600.2 [M+H]⁺.

### Intermediate 234

### N-[(7-methoxy-1-{[2-(trimethylsilyl)ethoxy]methyl}-1H-benzimidazol-2-yl)methyl]-2-(morpholin-4-yl)-8-(1,3-thiazol-2-yl)pyrazolo[1,5-a][1,3,5]triazin-4-amine

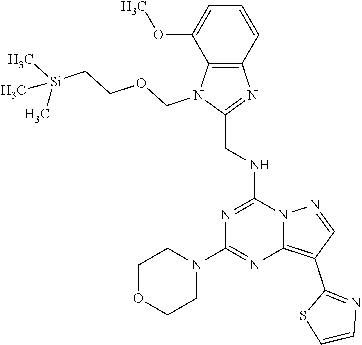 [SMILES: COc1cccc2nc(CNc3nc(N4CCOCC4)nc4c(-c5nccs5)cnn34)n(COCC[Si](C)(C)C)c12]

To a solution of 2-(morpholin-4-yl)-8-(1,3-thiazol-2-yl)pyrazolo[1,5-a][1,3,5]triazin-4-amine (Intermediate 232, 200 mg, 0.659 mmol) in N,N-dimethylformamide (2.0 mL) was added sodium hydride (42.2 mg, 1.05 mmol, 60% purity in mineral oil) at room temperature. After stirring at 60° C. for 15 minutes, 2-(chloromethyl)-7-methoxy-1-{[2-(trimethylsilyl)ethoxy]methyl}-1H-benzimidazole (Intermediate 189, 216 mg, 0.659 mmol) was added in one portion. The reaction mixture was stirred at room temperature for 16 hours. The mixture was quenched with water and extracted with ethyl acetate. The organic phase was washed with water and brine, dried over anhydrous sodium sulfate, and concentrated to give N-[(7-methoxy-1-{[2-(trimethylsilyl)ethoxy]methyl}-1H-benzimidazol-2-yl)methyl]-2-(morpholin-4-yl)-8-(1,3-thiazol-2-yl)pyrazolo[1,5-a][1,3,5]triazin-4-amine (400 mg, 67% purity, 68% yield) as yellow oil.

LC-MS (Method D): Rₜ=0.816 min; MS (ESIpos): m/z=594.2 [M+H]⁺.

### Intermediate 235

### benzyl 4-(4-{bis[(4-methoxyphenyl)methyl]amino}-8-iodopyrazolo[1,5-a][1,3,5]triazin-2-yl)piperazine-1-carboxylate

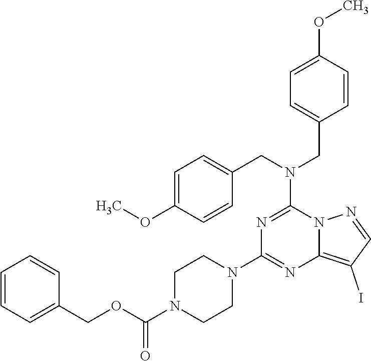 [SMILES: COc1ccc(CN(Cc2ccc(OC)cc2)c2nc(N3CCN(C(=O)OCc4ccccc4)CC3)nc3c(I)cnn23)cc1]

To a solution of 8-iodo-2-(methanesulfonyl)-N,N-bis[(4-methoxyphenyl)methyl]pyrazolo[1,5-a][1,3,5]triazin-4-amine (Intermediate 127, 40.0 g, 74% purity, 51.1 mmol) and benzyl piperazine-1-carboxylate (22.5 g, 102 mmol) in acetonitrile (500 mL) was added N,N-diisopropylethylamine (19.8 g, 153 mmol) at room temperature. After stirring at 70° C. for 16 hours, the reaction mixture was concentrated to 200 mL. The resulting suspension was cooled down and filtered. The filter cake was triturated with methyl tert-butyl ether (200 mL), and filtered. The filter cake was dried to give benzyl 4-{4-[bis(4-methoxybenzyl)amino]-8-iodopyrazolo[1,5-a][1,3,5]triazin-2-yl}piperazine-1-carboxylate (40.0 g, 90% purity, 98% yield) as a white solid.

LC-MS (Method C): Rₜ=1.21 min; MS (ESIpos): m/z=720.5 [M+H]⁺.

### Intermediate 236

### benzyl 4-[4-{bis[(4-methoxyphenyl)methyl]amino}-8-(1,3-thiazol-2-yl)pyrazolo[1,5-a][1,3,5]triazin-2-yl]piperazine-1-carboxylate

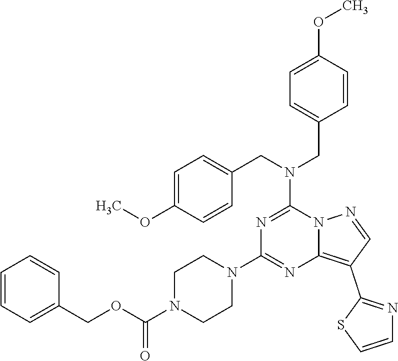 [SMILES: COc1ccc(CN(Cc2ccc(OC)cc2)c2nc(N3CCN(C(=O)OCc4ccccc4)CC3)nc3c(-c4nccs4)cnn23)cc1]

To a solution of 1,3-thiazole (946 mg, 11.1 mmol) in tetrahydrofuran (50 mL) was added n-butyllithium (4.4 mL, 2.5 M in hexane, 11 mmol) at −65° C. under nitrogen atmosphere. After stirring at −65° C. for 10 minutes under nitrogen atmosphere, zinc dichloride (2.84 g, 20.8 mmol) was added to the above mixture at −65° C.˜−60° C. under nitrogen atmosphere. The reaction mixture was warmed to room temperature. Benzyl 4-(4-{bis[(4-methoxyphenyl)methyl]amino}-8-iodopyrazolo[1,5-a][1,3,5]triazin-2-yl)piperazine-1-carboxylate (Intermediate 235, 5.00 g, 6.95 mmol) and tetrakis(triphenylphosphine)palladium(0) (401 mg, 0.35 mmol) were added to the above mixture at room temperature. After stirring at 60° C. for 16 hours under nitrogen atmosphere, the reaction mixture was quenched with water and extracted with ethyl acetate. The organic phase was washed with water and brine, dried over anhydrous sodium sulfate, and concentrated to give benzyl 4-{4-[bis(4-methoxybenzyl)amino]-8-(1,3-thiazol-2-yl)pyrazolo[1,5-a][1,3,5]triazin-2-yl}piperazine-1-carboxylate (5.00 g, 89% purity, 95% yield) as a yellow solid.

LC-MS (Method D): Rₜ=0.95 min; MS (ESIpos): m/z=677.2 [M+H]⁺.

### Intermediate 237

### benzyl 4-[4-{[(4-methoxyphenyl)methyl]amino}-8-(1,3-thiazol-2-yl)pyrazolo[1,5-a][1,3,5]triazin-2-yl]piperazine-1-carboxylate

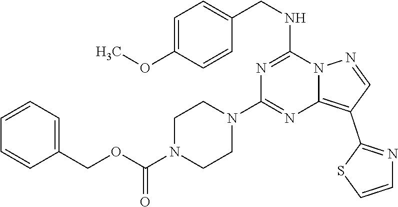 [SMILES: COc1ccc(CNc2nc(N3CCN(C(=O)OCc4ccccc4)CC3)nc3c(-c4nccs4)cnn23)cc1]

A solution of benzyl 4-[4-{bis[(4-methoxyphenyl)methyl]amino}-8-(1,3-thiazol-2-yl)pyrazolo[1,5-a][1,3,5]triazin-2-yl]piperazine-1-carboxylate (Intermediate 236, 5.00 g, 7.39 mmol) in trifluoroacetic acid (10.0 mL, 13 mmol) was stirred at room temperature for 16 hours. The reaction mixture was added to 100 mL saturated sodium bicarbonate aqueous solution slowly at 0° C., and extracted with ethyl acetate. The organic phase was washed with water and brine, dried over anhydrous sodium sulfate, and concentrated to give a residue. The residue was purified by column chromatography (1000 mesh, petroleum ether: ethyl acetate=5:1 then 1:1) to give a crude solid. The solid was triturated with 30 mL ethyl acetate and filtered to give benzyl 4-{4-[(4-methoxybenzyl)amino]-8-(1,3-thiazol-2-yl)pyrazolo[1,5-a][1,3,5]triazin-2-yl}piperazine-1-carboxylate (fraction1, 620 mg, 95% purity, 15% yield) as a white solid. The filtrate was concentrated and purified by preparative HPLC [lnstrument:ACSWH-GX-O; Column: Phenomenex Luna C18 150*25 mm*10 μm; eluent A: water (0.225% formic acid), eluent B: acetonitrile; gradient: 0-11 min 54-84% B; flow 25 mL/min; temperature: RT; Detector: UV 220/254 nm] to give benzyl 4-{4-[(4-methoxybenzyl)amino]-8-(1,3-thiazol-2-yl)pyrazolo[1,5-a][1,3,5]triazin-2-yl}piperazine-1-carboxylate (fraction2, 120 mg, 97% purity) as a white solid.

LC-MS (Method D): Rₜ=0.88 min; MS (ESIpos): m/z=557.2 [M+H]⁺.

### Intermediate 238

### benzyl 4-[4-{[(4,5-difluoro-1-{[2-(trimethylsilyl)ethoxy]methyl}-1H-benzimidazol-2-yl)methyl][(4-methoxyphenyl)methyl]amino}-8-(1,3-thiazol-2-yl)pyrazolo[1,5-a][1,3,5]triazin-2-yl]piperazine-1-carboxylate

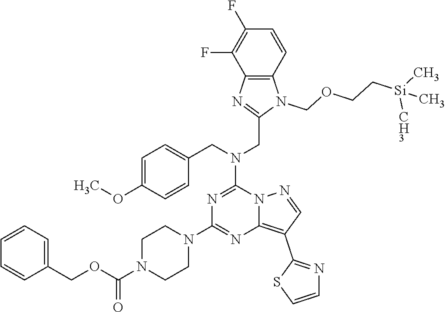 [SMILES: COc1ccc(CN(Cc2nc3c(F)c(F)ccc3n2COCC[Si](C)(C)C)c2nc(N3CCN(C(=O)OCc4ccccc4)CC3)nc3c(-c4nccs4)cnn23)cc1]

To a solution of benzyl 4-[4-{[(4-methoxyphenyl)methyl]amino}-8-(1,3-thiazol-2-yl)pyrazolo[1,5-a][1,3,5]triazin-2-yl]piperazine-1-carboxylate (Intermediate 237, 150 mg, 0.269 mmol) in N,N-dimethylformamide (4.0 mL) were added potassium carbonate (74.5 mg, 0.54 mmol) and 2-(chloromethyl)-4,5-difluoro-1-{[2-(trimethylsilyl)ethoxy]methyl}-1H-benzimidazole (Intermediate 158, 135 mg, 0.404 mmol) at room temperature. After stirring at 50° C. for 16 hours, the reaction mixture was diluted with water and extracted with ethyl acetate. The organic phase was washed with water and brine, dried over anhydrous sodium sulfate, and concentrated to give a residue. The residue was purified by flash chromatography (petroleum ether: ethyl acetate=1:0 to 1:1) to give benzyl 4-[4-{[(4,5-difluoro-1-{[2-(trimethylsilyl)ethoxy]methyl}-1H-benzimidazol-2-yl)methyl](4-methoxybenzyl)amino}-8-(1,3-thiazol-2-yl)pyrazolo[1,5-a][1,3,5]triazin-2-yl]piperazine-1-carboxylate (230 mg, 96% purity, 96% yield) as yellow oil.

LC-MS (Method D): Rₜ=1.03 min; MS (ESIpos): m/z=853.2 [M+H]⁺.

### Intermediate 239

### N-[(4,5-difluoro-1-{[2-(trimethylsilyl)ethoxy]methyl}-1H-benzimidazol-2-yl)methyl]-N-[(4-methoxyphenyl)methyl]-2-(4-methylpiperazin-1-yl)-8-(1,3-thiazol-2-yl)pyrazolo[1,5-a][1,3,5]triazin-4-amine

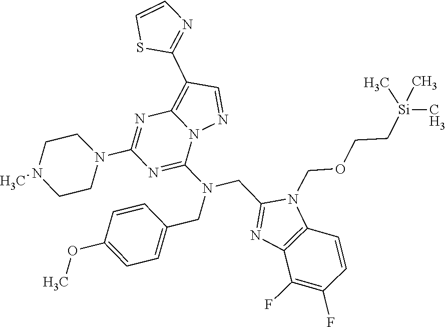

To a solution of benzyl 4-[4-{[(4,5-difluoro-1-{[2-(trimethylsilyl)ethoxy]methyl}-1H-benzimidazol-2-yl)methyl][(4-methoxyphenyl)methyl]amino}-8-(1,3-thiazol-2-yl)pyrazolo[1,5-a][1,3,5]triazin-2-yl]piperazine-1-carboxylate (Intermediate 238, 210 mg, 96% purity, 0.23 mmol) in methanol (4.5 mL) and tetrahydrofuran (4.5 mL) were added paraformaldehyde (0.71 mL, 37% purity, 0.95 mmol) and palladium (210 mg, 10% on activated carbon) at room temperature. After stirring at room temperature for 16 hours under hydrogen atmosphere (15 psi), the reaction mixture was filtered. The filtrate was purified by preparative TLC (dichloromethane: ethanol=10:1) to give N-[(4,5-difluoro-1-{[2-(trimethylsilyl)ethoxy]methyl}-1H-benzimidazol-2-yl)methyl]-N-(4-methoxybenzyl)-2-(4-methylpiperazin-1-yl)-8-(1,3-thiazol-2-yl)pyrazolo[1,5-a][1,3,5]triazin-4-amine (50 mg, 60% purity, 17% yield) as light brown oil.

LC-MS (Method C): Rₜ=0.95 min; MS (ESIpos): m/z=733.4 [M+H]⁺.

### Intermediate 240

### benzyl 4-[4-{[(4-methoxyphenyl)methyl][(7-methoxy-1-{[2-(trimethylsilyl)ethoxy]methyl}-1H-benzimidazol-2-yl)methyl]amino}-8-(1,3-thiazol-2-yl)pyrazolo[1,5-a][1,3,5]triazin-2-yl]piperazine-1-carboxylate

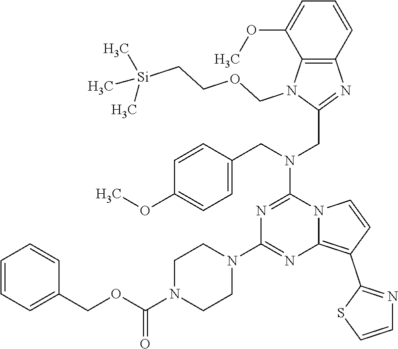 [SMILES: COc1ccc(CN(Cc2nc3cccc(OC)c3n2COCC[Si](C)(C)C)c2nc(N3CCN(C(=O)OCc4ccccc4)CC3)nc3c(-c4nccs4)ccn23)cc1]

To a solution of benzyl 4-[4-{[(4-methoxyphenyl)methyl]amino}-8-(1,3-thiazol-2-yl)pyrazolo[1,5-a][1,3,5]triazin-2-yl]piperazine-1-carboxylate (Intermediate 237, 150 mg, 0.27 mmol) in N,N-dimethylformamide (4.0 mL) were added potassium carbonate (74.5 mg, 0.54 mmol) and 2-(chloromethyl)-7-methoxy-1-{[2-(trimethylsilyl)ethoxy]methyl}-1H-benzimidazole (Intermediate 189, 132 mg, 0.40 mmol) at room temperature. After stirring at 50° C. for 16 hours, the reaction mixture was diluted with water and extracted with ethyl acetate. The organic phase was washed with water and brine, dried over anhydrous sodium sulfate, and concentrated to give a residue. The residue was purified by flash chromatography (petroleum ether: ethyl acetate=1:0 to 1:1) to give benzyl 4-[4-{(4-methoxybenzyl)[(7-methoxy-1-{[2-(trimethylsilyl)ethoxy]methyl}-1H-benzimidazol-2-yl)methyl]amino}-8-(1,3-thiazol-2-yl)pyrazolo [1,5-a][1,3,5]triazin-2-yl]piperazine-1-carboxylate (240 mg, 75% purity, 79% yield) as yellow oil.

LC-MS (Method D): Rₜ=0.97 min; MS (ESIpos): m/z=847.3 [M+H]⁺.

### Intermediate 241

### N-[(4-methoxyphenyl)methyl]-N-[(7-methoxy-1-{[2-(trimethylsilyl)ethoxy]methyl}-1H-benzimidazol-2-yl)methyl]-2-(4-methylpiperazin-1-yl)-8-(1,3-thiazol-2-yl)pyrazolo[1,5-a][1,3,5]triazin-4-amine

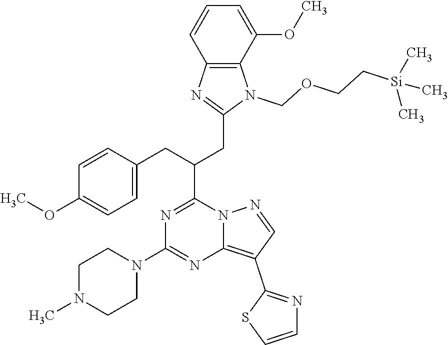

To a solution of benzyl 4-[4-{[(4-methoxyphenyl)methyl][(7-methoxy-1-{[2-(trimethylsilyl)ethoxy] methyl}-1H-benzimidazol-2-yl)methyl]amino}-8-(1,3-thiazol-2-yl)pyrazolo[1,5-a][1,3,5]triazin-2-yl]piperazine-1-carboxylate (Intermediate 240, 210 mg, 75% purity, 0.19 mmol) in tetrahydrofuran (5.0 mL) and methanol (5.0 mL) were added paraformaldehyde (67.0 mg, 0.74 mmol) and palladium (200 mg, 10% in activated carbon) at room temperature. After stirring at room temperature for 16 hours, the reaction mixture was filtered. The filtrate was purified by preparative TLC (dichloromethane:ethanol=10:1) to give N-(4-methoxybenzyl)-N-[(7-methoxy-1-{[2-(trimethylsilyl)ethoxy]methyl}-1H-benzimidazol-2-yl)methyl]-2-(4-methylpiperazin-1-yl)-8-(1,3-thiazol-2-yl)pyrazolo[1,5-a][1,3,5]triazin-4-amine (30.0 mg, 83% purity) as light brown oil.

LC-MS (Method C): Rₜ=0.91 min; MS (ESIpos): m/z=727.4 [M+H]⁺.

### Intermediate 242 [4-{bis[(4-methoxyphenyl)methyl]amino}-2-(morpholin-4-yl)pyrazolo[1,5-a][1,3,5]triazin-8-yl]boronic acid

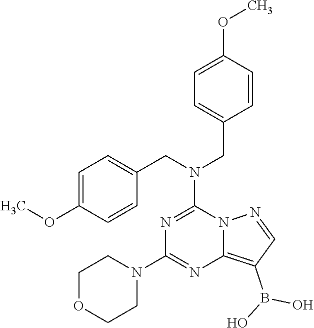 [SMILES: COc1ccc(CN(Cc2ccc(OC)cc2)c2nc(N3CCOCC3)nc3c(B(O)O)cnn23)cc1]

To a solution of 8-iodo-N,N-bis[(4-methoxyphenyl)methyl]-2-(morpholin-4-yl)pyrazolo[1,5-a][1,3,5]triazin-4-amine (Intermediate 128, 5.00 g, 8.53 mmol) in tetrahydrofuran (100 mL) was added 2-propylmagnesium chloride (8.5 mL, 17 mmol, 2 M in tetrahydrofuran) dropwise at 0° C. under nitrogen atmosphere. After stirring at room temperature for 1 hour under nitrogen atmosphere, trimethyl borate (3.8 mL, 34 mmol) was added to the above mixture at −65° C. The reaction mixture was stirred at room temperature for 16 hours under nitrogen atmosphere. The mixture was quenched with 50 mL of saturated ammonium chloride aqueous solution and diluted with water, extracted with tetrahydrofuran. The combined organic phase dried over anhydrous sodium sulfate and used directly in the next step without further purification.

LC-MS (Method C): Rₜ=0.96 min; MS (ESIpos): m/z=505.0 [M+H]⁺.

### Intermediate 243

### N,N-bis[(4-methoxyphenyl)methyl]-2-(morpholin-4-yl)-8-(pyridazin-4-yl)pyrazolo[1,5-a][1,3,5]triazin-4-amine

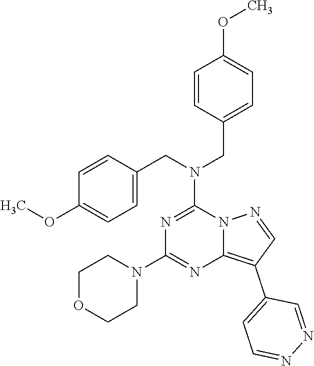 [SMILES: COc1ccc(CN(Cc2ccc(OC)cc2)c2nc(N3CCOCC3)nc3c(-c4ccnnc4)cnn23)cc1]

To a solution of [4-{bis[(4-methoxyphenyl)methyl]amino}-2-(morpholin-4-yl)pyrazolo[1,5-a][1,3,5]triazin-8-yl]boronic acid (Intermediate 242, 4.30 g, 8.53 mmol) and 4-bromopyridazine (1.63 g, 10.2 mmol) in tetrahydrofuran (60 mL) were added methanesulfonato(2-dicyclohexylphosphino-2′,4′,6′-tri-i-propyl-1,1′-biphenyl)(2′-amino-1,1′-biphenyl-2-yl)palladium(II) (361 mg, 0.426 mmol) and potassium phosphate (13 mL, 2.0 M in water) at room temperature. After stirring at 60° C. for 16 hours under nitrogen atmosphere, the reaction mixture was diluted with water and extracted with ethyl acetate. The organic phase was washed with water and brine, dried over anhydrous sodium sulfate, and concentrated to give a residue. The residue was purified by silica gel column chromatography (1000 mesh, petroleum ether: ethyl acetate=5:1, then 0:1, then dichloromethane: ethyl acetate=1:1) to give N,N-bis(4-methoxybenzyl)-2-(morpholin-4-yl)-8-(pyridazin-4-yl)pyrazolo[1,5-a][1,3,5]triazin-4-amine (1.37 g, 30% yield) as yellow oil.

LC-MS (Method D): Rₜ=0.808 min; MS (ESIpos): m/z=539.2 [M+H]⁺.

### Intermediate 244

### N-[(4-methoxyphenyl)methyl]-2-(morpholin-4-yl)-8-(pyridazin-4-yl)pyrazolo[1,5-a][1,3,5]triazin-4-amine

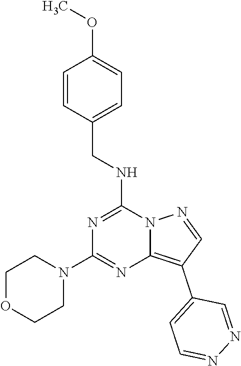 [SMILES: COc1ccc(CNc2nc(N3CCOCC3)nc3c(-c4ccnnc4)cnn23)cc1]

A solution of N,N-bis[(4-methoxyphenyl)methyl]-2-(morpholin-4-yl)-8-(pyridazin-4-yl)pyrazolo [1,5-a][1,3,5]triazin-4-amine (Intermediate 243, 1.37 g, 2.54 mmol) in trifluoroacetic acid (10 mL) was stirred at room temperature for 2 hours. The reaction mixture was poured into cooled saturated sodium bicarbonate aqueous solution, extracted with ethyl acetate. The organic phase was washed with brine, dried over anhydrous sodium sulfate, concentrated to give a residue. The residue was purified by silica gel column chromatography (1000 mesh, petroleum ether: ethyl acetate=5:1 then 0:1) to give N-(4-methoxybenzyl)-2-(morpholin-4-yl)-8-(pyridazin-4-yl)pyrazolo[1,5-a][1,3,5]triazin-4-amine (1.00 g, 94% yield) as yellow oil.

LC-MS (Method D): Rₜ=0.714 min; MS (ESIpos): m/z=419.1 [M+H]⁺.

### Intermediate 245

### N-[(4,5-difluoro-1-{[2-(trimethylsilyl)ethoxy]methyl}-1H-benzimidazol-2-yl)methyl]-N-[(4-methoxyphenyl)methyl]-2-(morpholin-4-yl)-8-(pyridazin-4-yl)pyrazolo[1,5-a][1,3,5]triazin-4-amine

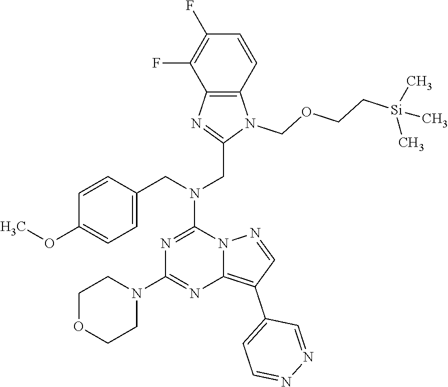

To a solution of N-[(4-methoxyphenyl)methyl]-2-(morpholin-4-yl)-8-(pyridazin-4-yl)pyrazolo[1,5-a][1,3,5]triazin-4-amine (Intermediate 244, 200 mg, 0.48 mmol) in N,N-dimethylformamide (4.0 mL) were added potassium carbonate (132 mg, 0.96 mmol) and 2-(chloromethyl)-4,5-difluoro-1-{[2-(trimethylsilyl)ethoxy]methyl}-1H-benzimidazole (Intermediate 158, 193 mg, 99% purity, 0.57 mmol) at room temperature. After stirring at 50° C. for 16 hours, the mixture was diluted with ethyl actate and washed with water and brine. The organic phase was dried over anhydrous sodium sulfate and concentrated to give a residue. The residue was purified by TLC (ethyl actate) to give N-[(4,5-difluoro-1-{[2-(trimethylsilyl)ethoxy]methyl}-1H-benzimidazol-2-yl)methyl]-N-(4-methoxybenzyl)-2-(morpholin-4-yl)-8-(pyridazin-4-yl)pyrazolo[1,5-a][1,3,5]triazin-4-amine (150 mg, 90% purity, 40% yield) as yellow oil.

LC-MS (Method C): Rₜ=0.96 min; MS (ESIpos): m/z=715.3 [M+H]⁺.

### Intermediate 246

### N,N-bis[(4-methoxyphenyl)methyl]-8-(1-methyl-1H-pyrazol-4-yl)-2-(morpholin-4-yl)pyrazolo[1,5-a][1,3,5]triazin-4-amine

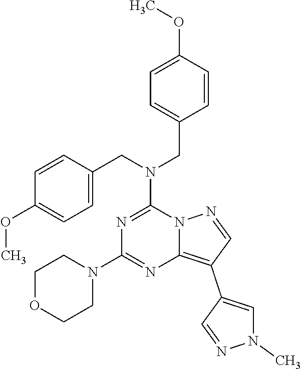 [SMILES: COc1ccc(CN(Cc2ccc(OC)cc2)c2nc(N3CCOCC3)nc3c(-c4cnn(C)c4)cnn23)cc1]

To a solution of 8-iodo-N,N-bis[(4-methoxyphenyl)methyl]-2-(morpholin-4-yl)pyrazolo[1,5-a][1,3,5]triazin-4-amine (Intermediate 128, 10.0 g, 17.1 mmol) and (1-methyl-1H-pyrazol-4-yl)boronic acid (4.30 g, 34.1 mmol) in tetrahydrofuran (100 mL) were added methanesulfonato(2-dicyclohexylphosphino-2′,4′,6′-tri-i-propyl-1,1′-biphenyl)(2′-amino-1,1′-biphenyl-2-yl)palladium(II) (722 mg, 850 mmol) and potassium phosphate (25.6 mL, 51.1 mmol, 2.0 M in water) at room temperature. After stirring at 60° C. for 16 hours, the reaction mixture was diluted with water, extracted with ethyl acetate. The organic phase was washed with brine, dried over anhydrous sodium sulfate, filtered and concentrated to give a residue. The residue was purified by column chromatography (1000 mesh, petroleum ether: ethyl acetate=5:1 then 1:1) to give N,N-bis(4-methoxybenzyl)-8-(1-methyl-1H-pyrazol-4-yl)-2-(morpholin-4-yl)pyrazolo[1,5-a][1,3,5]triazin-4-amine (5.20 g, 56% yield) as yellow oil.

LC-MS (Method D): Rₜ=0.97 min; MS (ESIpos): m/z=541.2 [M+H]⁺.

### Intermediate 247

### N-[(4-methoxyphenyl)methyl]-8-(1-methyl-1H-pyrazol-4-yl)-2-(morpholin-4-yl)pyrazolo[1,5-a][1,3,5]triazin-4-amine

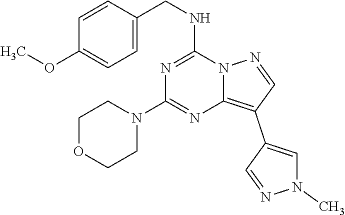

A solution of N,N-bis[(4-methoxyphenyl)methyl]-8-(1-methyl-1H-pyrazol-4-yl)-2-(morpholin-4-yl)pyrazolo[1,5-a][1,3,5]triazin-4-amine (Intermediate 246, 5.70 g, 10.5 mmol) in trifluoroacetic acid (60 mL) was stirred at 40° C. for 3 hours. The reaction mixture was concentrated to give a residue. The residue was poured into water, adjusted to pH=7 by saturated sodium bicarbonate aqueous solution. The mixture was filtered. The filter cake was washed with water and dried under vacuum to give N-(4-methoxybenzyl)-8-(1-methyl-1H-pyrazol-4-yl)-2-(morpholin-4-yl)pyrazolo[1,5-a][1,3,5]triazin-4-amine as a brown solid (1.30 g, 95% purity, 28% yield) as a brown solid. The filtrate was extracted with ethyl acetate. The organic phase was washed with water and brine, dried over anhydrous sodium sulfate and filtered. The filtrate was concentrated and purified by flash chromatography (petroleum ether: ethyl acetate=10:1 to 1:1) to give N-(4-methoxybenzyl)-8-(1-methyl-1H-pyrazol-4-yl)-2-(morpholin-4-yl)pyrazolo[1,5-a][1,3,5]triazin-4-amine as a brown solid (3.90 g, 60% purity, 53% yield) as a white solid.

LC-MS (Method D): Rₜ=0.83 min; MS (ESIpos): m/z=421.2 [M+H]⁺.

¹H NMR (400 MHz, DMSO-d₆) 5 [ppm]=8.97 (t, J=6.4 Hz, 1H), 8.15 (s, 1H), 7.97 (s, 1H), 7.81 (s, 1H), 7.34 (d, J=8.8 Hz, 2H), 6.88 (d, J=8.8 Hz, 2H), 4.56 (d, J=2.4 Hz, 2H), 3.85 (s, 3H), 3.78 (t, J=4.4 Hz, 4H), 3.72 (s, 3H), 3.66 (t, J=4.8 Hz, 4H).

### Intermediate 248

### N-[(4,5-difluoro-1-{[2-(trimethylsilyl)ethoxy]methyl}-1H-benzimidazol-2-yl)methyl]-N-(4-methoxybenzyl)-8-(1-methyl-1H-pyrazol-4-yl)-2-(morpholin-4-yl)pyrazolo[1,5-a][1,3,5]triazin-4-amine

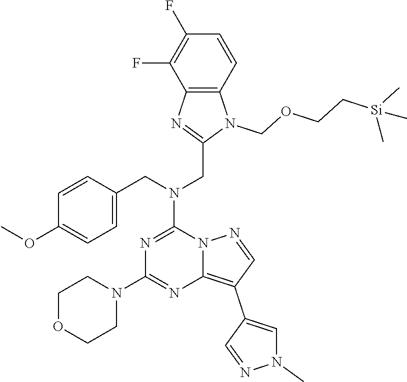

The reaction was performed in two separate batches. To a solution of N-(4-methoxybenzyl)-8-(1-methyl-1H-pyrazol-4-yl)-2-(morpholin-4-yl)pyrazolo [1,5-a][1,3,5]triazin-4-amine (Intermediate 247, 100/200 mg, 0.238/0.476 mmol) in N,N-dimethylformamide (2.0/4.0 mL) was added sodium hydride (14.25/28.5 mg, 0.357/0.713 mmol, 60% purity in mineral oil) at room temperature. After stirring at 60° C. for 15 minutes, (4,5-difluoro-1-{[2-(trimethylsilyl) ethoxy]methyl}-1H-benzimidazol-2-yl)methanol (Intermediate 158, 97/194 mg, 94% purity, 0.309/0.618 mmol) was added in a portion. After stirring at room temperature for 16 hours, the reaction mixtures were combined, diluted with water and extracted with ethyl acetate. The combined organic layers were washed with water and brine, dried over anhydrous sodium sulfate and concentrated to give a crude product. The crude product was purified by preparative TLC (petroleum ether: ethyl acetate=2:1) to give N-[(4,5-difluoro-1-{[2-(trimethylsilyl)ethoxy]methyl}-1H-benzimidazol-2-yl)methyl]-N-(4-methoxybenzyl)-8-(1-methyl-1H-pyrazol-4-yl)-2-(morpholin-4-yl)pyrazolo[1,5-a][1,3,5]triazin-4-amine (108 mg, 91% purity, 29% yield) as light yellow oil.

LC-MS (Method D): Rₜ=1.02 min; MS (ESIpos): m/z=717.2 [M+H]⁺.

### Intermediate 249

### 5,6-dichloro-2-(chloromethyl)-1H-benzimidazole

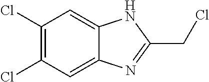 [SMILES: ClCc1nc2cc(Cl)c(Cl)cc2[nH]1]

A solution of 4,5-dichlorobenzene-1,2-diamine (10.0 g, 56.5 mmol) and chloroacetic acid (5.1 mL, 85 mmol) in hydrochloric acid (100 mL, 6.0 M, 600 mmol) was stirred at 100° C. for 16 hours. The reaction mixture was cooled to room temperature. Saturated sodium carbonate solution was added to the reaction mixture to adjust pH=7-8. The mixture was filtered. The filter cake was washed with water, and dried to give 5,6-dichloro-2-(chloromethyl)-1H-benzimidazole (12.3 g, 92% yield) as a red solid.

LC-MS (Method D): Rₜ=0.73 min; MS (ESIpos): m/z=237.0 [M+H]⁺.

### Intermediate 250

### 5,6-dichloro-2-(chloromethyl)-1-{[2-(trimethylsilyl)ethoxy]methyl}-1H-benzimidazole

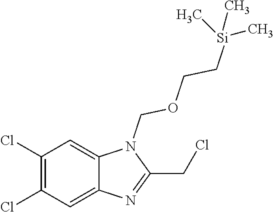 [SMILES: C[Si](C)(C)CCOCn1c(CCl)nc2cc(Cl)c(Cl)cc21]

To a solution of 5,6-dichloro-2-(chloromethyl)-1H-benzimidazole (Intermediate 249, 17.3 g, 95% purity, 70.1 mmol) and N,N-diisopropylethylamine (24 mL, 140 mmol) in tetrahydrofuran (250 mL) was added [2-(chloromethoxy)ethyl](trimethyl)silane (14.0 g, 84.1 mmol) at room temperature. After stirring at room temperature for 16 hours, the reaction mixture was diluted with water and extracted with ethyl acetate. The combined organic layers were washed with water and brine, dried over anhydrous sodium sulfate and concentrated to give a crude product. The crude product was purified by silica gel chromatography column (200-300 mesh, petroleum ether: ethyl acetate=20:1 then 10:1 then 5:1) to give 5,6-dichloro-2-(chloromethyl)-1-{[2-(trimethylsilyl)ethoxy]methyl}-1H-benzimidazole (14.5 g, 86% purity, 48% yield) as a brown solid.

LC-MS (Method D): Rₜ=1.03 min; MS (ESIpos): m/z=367.0 [M+H]⁺.

### Intermediate 251

### N-[(5,6-dichloro-1-{[2-(trimethylsilyl)ethoxy]methyl}-1H-benzimidazol-2-yl)methyl]-N-[(4-methoxyphenyl)methyl]-8-(1-methyl-1H-pyrazol-4-yl)-2-(morpholin-4-yl)pyrazolo[1,5-a][1,3,5]triazin-4-amine

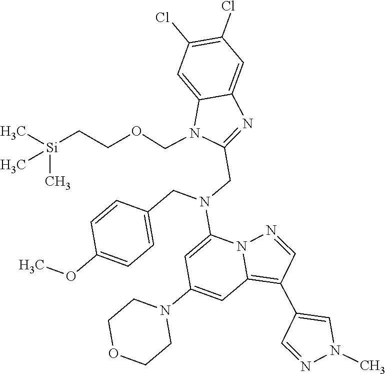

To a solution of N-[(4-methoxyphenyl)methyl]-8-(1-methyl-1H-pyrazol-4-yl)-2-(morpholin-4-yl)pyrazolo[1,5-a][1,3,5]triazin-4-amine (Intermediate 247, 200 mg, 0.476 mmol) in N,N-dimethylformamide (4.0 mL) was added sodium hydride (28.5 mg, 0.713 mmol, 60% purity in mineral oil) at room temperature. After stirring at 60° C. for 15 minutes, 5,6-dichloro-1-{[2-(trimethylsilyl)ethoxy]methyl}-1H-benzimidazol-2-yl)methanol (Intermediate 250, 215 mg, 0.618 mmol) was added in a portion. After stirring at room temperature for 16 hours, the reaction mixture was diluted with water and extracted with ethyl acetate. The combined organic layers were washed with water and brine, dried over anhydrous sodium sulfate and concentrated to give a crude product. The crude product was purified by preparative TLC (petroleum ether: ethyl acetate=2:1) to give N-[(5,6-dichloro-1-{[2-(trimethylsilyl) ethoxy]methyl}-1H-benzimidazol-2-yl)methyl]-N-(4-methoxybenzyl)-8-(1-methyl-1H-pyrazol-4-yl)-2-(morpholin-4-yl)pyrazolo[1,5-a][1,3,5]triazin-4-amine (157 mg, 91% purity, 40% yield) as light yellow oil.

LC-MS (Method C): Rₜ=1.18 min; MS (ESIpos): m/z=749.4 [M+H]⁺.

### Intermediate 252

### benzyl 4-[4-{bis[(4-methoxyphenyl)methyl]amino}-8-(1-methyl-1H-pyrazol-4-yl)pyrazolo[1,5-a][1,3,5]triazin-2-yl]piperazine-1-carboxylate

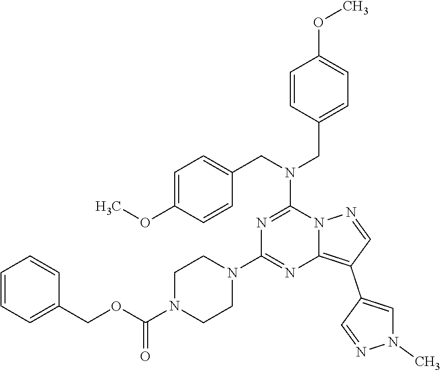 [SMILES: COc1ccc(CN(Cc2ccc(OC)cc2)c2nc(N3CCN(C(=O)OCc4ccccc4)CC3)nc3c(-c4cnn(C)c4)cnn23)cc1]

To a solution of benzyl 4-(4-{bis[(4-methoxyphenyl)methyl]amino}-8-iodopyrazolo[1,5-a][1,3,5]triazin-2-yl)piperazine-1-carboxylate (Intermediate 235, 3.00 g, 90% purity, 3.75 mmol) and (1-methyl-1H-pyrazol-4-yl)boronic acid (945 mg, 7.50 mmol) in tetrahydrofuran (54 mL) were added methanesulfonato(2-dicyclohexylphosphino-2′,4′,6′-tri-i-propyl-1,1′-biphenyl)(2′-amino-1,1′-biphenyl-2-yl)palladium(II) (159 mg, 0.19 mmol) and potassium phosphate (5.6 mL, 11 mmol, 2 M in water) at room temperature. After stirring at 70° C. for 16 hours under nitrogen atmosphere, the reaction mixture was diluted with water, extracted with ethyl acetate. The organic phase was washed with brine, dried over anhydrous sodium sulfate and concentrated to give a crude product. The crude product was purified by column chromatography on silica gel (petroleum ether: ethyl acetate=10:1, then 5:1, then 2:1) to give benzyl 4-{4-[bis(4-methoxybenzyl)amino]-8-(1-methyl-1H-pyrazol-4-yl)pyrazolo[1,5-a][1,3,5]triazin-2-yl}piperazine-1-carboxylate (1.58 g, 88% purity, 55% yield) as a brown solid.

LC-MS (Method C): Rₜ=1.13 min; MS (ESIpos): m/z=674.5 [M+H]⁺.

### Intermediate 253

### N-[(4-methoxyphenyl)methyl]-8-(1-methyl-1H-pyrazol-4-yl)-2-(piperazin-1-yl)pyrazolo[1,5-a][1,3,5]triazin-4-amine

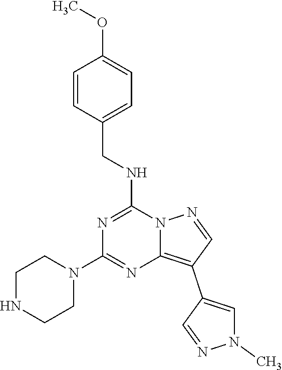 [SMILES: COc1ccc(CNc2nc(N3CCNCC3)nc3c(-c4cnn(C)c4)cnn23)cc1]

A solution of benzyl 4-[4-{bis[(4-methoxyphenyl)methyl]amino}-8-(1-methyl-1H-pyrazol-4-yl)pyrazolo[1,5-a][1,3,5]triazin-2-yl]piperazine-1-carboxylate (Intermediate 252, 1.58 g, 88% purity, 2.06 mmol) in trifluoroacetic acid (15 mL) was stirred at 80° C. for 3 hours. The mixture was concentrated to give N-[(4-methoxyphenyl)methyl]-8-(1-methyl-1H-pyrazol-4-yl)-2-(piperazin-1-yl)pyrazolo[1,5-a][1,3,5]triazin-4-amine (865 mg, 72% purity, 72% yield) as a yellow solid, used into next step directly.

LC-MS (Method D): Rₜ=0.68 min; MS (ESIpos): m/z=420.2 [M+H]⁺.

### Intermediate 254

### benzyl 4-[4-{[(4-methoxyphenyl)methyl]amino}-8-(1-methyl-1H-pyrazol-4-yl)pyrazolo[1,5-a][1,3,5]triazin-2-yl]piperazine-1-carboxylate

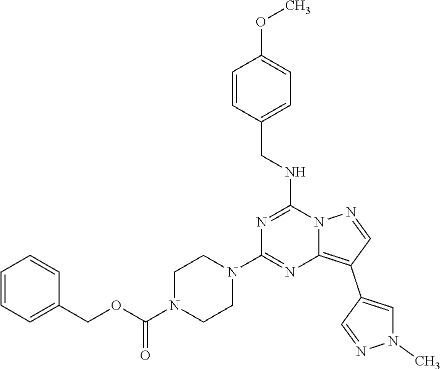 [SMILES: COc1ccc(CNc2nc(N3CCN(C(=O)OCc4ccccc4)CC3)nc3c(-c4cnn(C)c4)cnn23)cc1]

To a solution of N-[(4-methoxyphenyl)methyl]-8-(1-methyl-1H-pyrazol-4-yl)-2-(piperazin-1-yl)pyrazolo[1,5-a][1,3,5]triazin-4-amine (Intermediate 253, 866 mg, 72% purity, 1.49 mmol) and sodium hydrogen carbonate (500 mg, 5.94 mmol) in tetrahydrofuran (20 mL) and water (20 mL) was added benzyl carbonochloridate (355 mg, 2.08 mmol) at 0° C. The reaction mixture was diluted with water, extracted with ethyl acetate. The organic phase was washed with brine, dried over anhydrous sodium sulfate and concentrated to give a crude product. The crude product was purified by column chromatography on silica gel (petroleum ether: ethyl acetate=4:1, then 2:1, then 1:1 then 1:2) to give benzyl 4-{4-[(4-methoxybenzyl)amino]-8-(1-methyl-1H-pyrazol-4-yl)pyrazolo[1,5-a][1,3,5]triazin-2-yl}piperazine-1-carboxylate (650 mg, 80% purity, 63% yield) as a brown solid.

LC-MS (Method C): Rₜ=1.02 min; MS (ESIpos): m/z=554.4 [M+H]⁺.

### Intermediate 255

### benzyl 4-[4-{[(5,6-dichloro-1-{[2-(trimethylsilyl)ethoxy]methyl}-1H-benzimidazol-2-yl)methyl][(4-methoxyphenyl)methyl]amino}-8-(1-methyl-1H-pyrazol-4-yl)pyrazolo[1,5-a][1,3,5]triazin-2-yl]piperazine-1-carboxylate

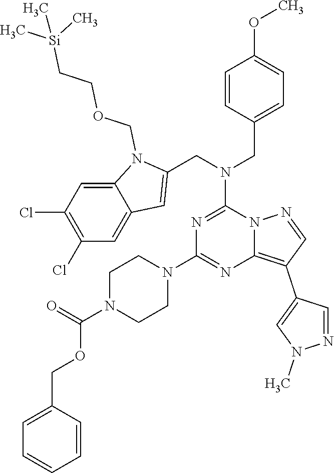 [SMILES: COc1ccc(CN(Cc2cc3cc(Cl)c(Cl)cc3n2COCC[Si](C)(C)C)c2nc(N3CCN(C(=O)OCc4ccccc4)CC3)nc3c(-c4cnn(C)c4)cnn23)cc1]

To a solution of benzyl 4-[4-{[(4-methoxyphenyl)methyl]amino}-8-(1-methyl-1H-pyrazol-4-yl)pyrazolo[1,5-a][1,3,5]triazin-2-yl]piperazine-1-carboxylate (Intermediate 254, 200 mg, 75% purity, 0.271 mmol), 5,6-dichloro-2-(chloromethyl)-1-{[2-(trimethylsilyl)ethoxy]methyl}-1H-benzimidazole (Intermediate 250, 138 mg, 86% purity, 0.325 mmol) in N,N-dimethylformamide (5.0 mL) was added potassium carbonate (74.9 mg, 0.541 mmol) at room temperature. After stirring at 50° C. for 16 hours, the reaction mixture was diluted with water and extracted with ethyl acetate. The combined organic layers were washed with water and brine, dried over anhydrous sodium sulfate and concentrated to give a residue. The residue was purified by preparative TLC (dichloromethane:methanol=10:1) to give benzyl 4-[4-{[(5,6-dichloro-1-{[2-(trimethylsilyl)ethoxy]methyl}-1H-benzimidazol-2-yl)methyl](4-methoxybenzyl)amino}-8-(1-methyl-1H-pyrazol-4-yl)pyrazolo[1,5-a][1,3,5]triazin-2-yl]piperazine-1-carboxylate (125 mg, 52% yield, 90% purity) as light brown oil.

LC-MS (Method C): Rₜ=1.10 min; MS (ESIpos): m/z=882.5 [M+H]⁺.

### Intermediate 256

### N-[(5,6-dichloro-1-{[2-(trimethylsilyl)ethoxy]methyl}-1H-benzimidazol-2-yl)methyl]-N-[(4-methoxyphenyl)methyl]-2-(4-methylpiperazin-1-yl)-8-(1-methyl-1H-pyrazol-4-yl)pyrazolo[1,5-a][1,3,5]triazin-4-amine

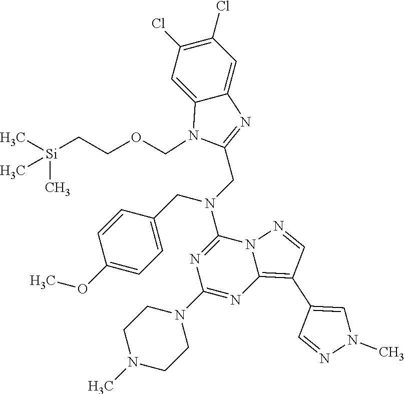

To a solution of benzyl 4-[4-{[(5,6-dichloro-1-{[2-(trimethylsilyl)ethoxy]methyl}-1H-benzimidazol-2-yl)methyl][(4-methoxyphenyl)methyl]amino}-8-(1-methyl-1H-pyrazol-4-yl)pyrazolo[1,5-a][1,3,5]triazin-2-yl]piperazine-1-carboxylate (Intermediate 255, 125 mg, 90% purity, 0.13 mmol) in tetrahydrofuran (4.0 mL) and methanol (4.0 mL) were added paraformaldehyde (45.9 mg, 0.51 mmol) and palladium (120 mg, 10% on activated carbon) at room temperature. After stirring at room temperature for 16 hours under hydrogen atmosphere (15 psi), the reaction mixture was filtered, and the filtrate was concentrated to give N-[(5,6-dichloro-1-{[2-(trimethylsilyl)ethoxy]methyl}-1H-benzimidazol-2-yl)methyl]-N-[(4-methoxyphenyl)methyl]-2-(4-methylpiperazin-1-yl)-8-(1-methyl-1H-pyrazol-4-yl)pyrazolo[1,5-a][1,3,5]triazin-4-amine (97.2 mg, 78% purity, 78% yield) as yellow oil, used into next step directly.

LC-MS (Method A): Rₜ=1.25 min; MS (ESIpos): m/z=762.4 [M+H]⁺.

### Intermediate 257

### 1-(difluoromethyl)-4-(4,4,5,5-tetramethyl-1,3,2-dioxaborolan-2-yl)-1H-pyrazole

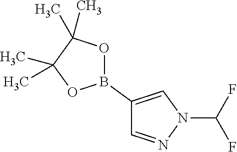 [SMILES: CC1(C)OB(c2cnn(C(F)F)c2)OC1(C)C]

A solution of 4-(4,4,5,5-tetramethyl-1,3,2-dioxaborolan-2-yl)-1H-pyrazole (5.00 g, 25.8 mmol), sodium chloro(difluoro)acetate (5.89 g, 38.7 mmol) and sodium carbonate (5.46 g, 51.5 mmol) in N,N-dimethylformamide (80 mL) was stirred at 80° C. for 5 hours. The reaction mixture was diluted with water and extracted with ethyl acetate. The combined organic layers were washed with water and brine, dried over anhydrous sodium sulfate and concentrated to give 1-(difluoromethyl)-4-(4,4,5,5-tetramethyl-1,3,2-dioxaborolan-2-yl)-1H-pyrazole (3.00 g, 90% purity, 43% yield) as brown oil.

LC-MS (Method C): Rₜ=0.76 min; MS (ESIpos): m/z=245.1 [M+H]⁺.

### Intermediate 258

### 8-[1-(difluoromethyl)-1H-pyrazol-4-yl]-N,N-bis[(4-methoxyphenyl)methyl]-2-(morpholin-4-yl)pyrazolo[1,5-a][1,3,5]triazin-4-amine

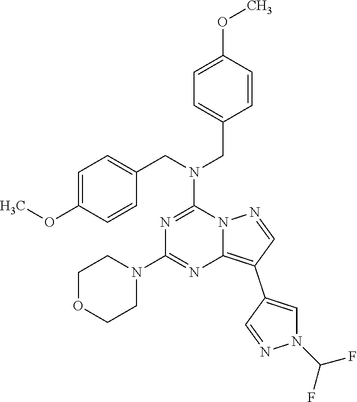 [SMILES: COc1ccc(CN(Cc2ccc(OC)cc2)c2nc(N3CCOCC3)nc3c(-c4cnn(C(F)F)c4)cnn23)cc1]

The reaction was performed in five separate batches. To a solution of 8-iodo-N,N-bis[(4-methoxyphenyl)methyl]-2-(morpholin-4-yl)pyrazolo[1,5-a][1,3,5]triazin-4-amine (Intermediate 128, 0.1 g/0.1 g/3.00 g/0.569 g/3.00 g, 167 μmol/167 μmol/5.01 mmol/950 μmol/5.01 mmol) and 1-(difluoromethyl)-4-(4,4,5,5-tetramethyl-1,3,2-dioxaborolan-2-yl)-1H-pyrazole (Intermediate 257, 70.3 mg/70.3 mg/1.99 g/400 mg/1.99 g, 250.6 μmol/250.6 μmol/7.52 mmol/1.43 mmol/7.52 mmol) in mixed solvent of water (0.1 mL/0.1 mL/3.0 mL/0.6 mL/3.0 mL) and tetrahydrofuran (2 mL/2 mL/60 mL/12 mL/60 mL) were added [1,1′ bis(diphenylphosphi n.)ferrocene]dichloropalladium(II) (6.1 mg/6.1 mg/183 mg/34.8 mg/183 mg, 8.4 mmol/8.4 μmol/0.025 mmol/47.5 mmol/0.25 mmol) and potassium carbonate (62.3 mg/62.3 mg/2.08 g/394.1 mg/2.08 g, 501.3 μmol/501.3 μmol/15.0 mmol/2.85 mmol/15.0 mmol) at room temperature. After stirring at 70° C. for 16 hours under nitrogen atmosphere (15 psi), the reaction mixtures were combined, diluted with water, extracted with ethyl acetate. The organic phase was washed with brine, dried over anhydrous sodium sulfate and filtered. The filtrate was concentrated and purified by silica gel column chromatography (1000 mesh, petroleum ether: ethyl acetate=1:0, then 20:1, then 10:1, then 5:1, then 3:1, then 2:1, then 1:1) to give 8-[1-(difluoromethyl)-1H-pyrazol-4-yl]-N,N-bis(4-methoxybenzyl)-2-(morpholin-4-yl)pyrazolo[1,5-a][1,3,5]triazin-4-amine (900 mg, 71% purity, 22% yield) as a brown solid.

LC-MS (Method C): Rₜ=1.09 min; MS (ESIpos): m/z=577.4 [M+H]⁺.

### Intermediate 259

### 8-[1-(difluoromethyl)-1H-pyrazol-4-yl]-N-[(4-methoxyphenyl)methyl]-2-(morpholin-4-yl)pyrazolo[1,5-a][1,3,5]triazin-4-amine

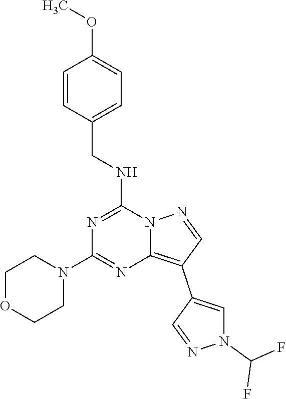 [SMILES: COc1ccc(CNc2nc(N3CCOCC3)nc3c(-c4cnn(C(F)F)c4)cnn23)cc1]

A solution of 8-[1-(difluoromethyl)-1H-pyrazol-4-yl]-N,N-bis[(4-methoxyphenyl)methyl]-2-(morpholin-4-yl)pyrazolo[1,5-a][1,3,5]triazin-4-amine (Intermediate 258, 900 mg, 1.11 mmol) in trifluoroacetic acid (6.0 mL) was stirred at 60° C. for 3 hours. The solution was diluted with water, and adjusted to pH=7 by sodium bicarbonate, and extracted with ethyl acetate. The organic phase was washed with brine, dried over anhydrous sodium sulfate and concentrated to give 8-[1-(difluoromethyl)-1H-pyrazol-4-yl]-N-(4-methoxybenzyl)-2-(morpholin-4-yl)pyrazolo[1,5-a][1,3,5]triazin-4-amine (890 mg, 56% purity, 98% yield) as a brown solid.

LC-MS (Method C): Rₜ=0.98 min; MS (ESIpos): m/z=457.3 [M+H]⁺.

### Intermediate 260

### 8-[1-(difluoromethyl)-1H-pyrazol-4-yl]-N-[(4-methoxyphenyl)methyl]-2-(morpholin-4-yl)-N-[(1-{[2-(trimethylsilyl)ethoxy]methyl}-1H-benzimidazol-2-yl)methyl]pyrazolo[1,5-a][1,3,5]triazin-4-amine

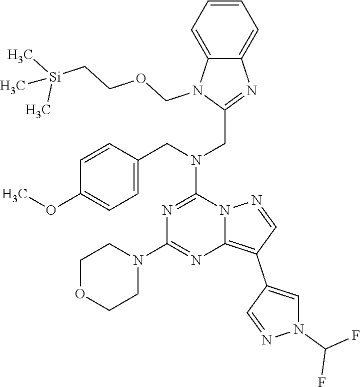 [SMILES: COc1ccc(CN(Cc2nc3ccccc3n2COCC[Si](C)(C)C)c2nc(N3CCOCC3)nc3c(-c4cnn(C(F)F)c4)cnn23)cc1]

To a solution of 8-[1-(difluoromethyl)-1H-pyrazol-4-yl]-N-[(4-methoxyphenyl)methyl]-2-(morpholin-4-yl)pyrazolo[1,5-a][1,3,5]triazin-4-amine (Intermediate 259, 245 mg, 0.30 mmol), 2-(chloromethyl)-1-{[2-(trimethylsilyl)ethoxy]methyl}-1H-benzimidazole (Intermediate 130, 180 mg, 0.51 mmol) in N,N-dimethylformamide (5.0 mL) was added potassium carbonate (124 mg, 0.90 mmol) at room temperature. After stirring at 50° C. for 16 hours, the reaction mixture was diluted with water and extracted with ethyl acetate. The combined organic layers were washed with water and brine, dried over anhydrous sodium sulfate and concentrated to give a residue. The residue was purified by preparative TLC (petroleum ether: ethyl acetate=1:1) to give 8-[1-(difluoromethyl)-1H-pyrazol-4-yl]-N-(4-methoxybenzyl)-2-(morpholin-4-yl)-N-[(1-{[2-(trimethylsilyl)ethoxy]methyl}-1H-benzimidazol-2-yl)methyl]pyrazolo[1,5-a][1,3,5]triazin-4-amine (150 mg, 84% purity, 59% yield) as yellow oil.

LC-MS (Method C): Rₜ=1.03 min; MS (ESIpos): m/z=717.5 [M+H]⁺.

### Intermediate 261

### 8-[1-(difluoromethyl)-1H-pyrazol-4-yl]-N-[(4,5-difluoro-1-{[2-(trimethylsilyl)ethoxy]methyl}-1H-benzimidazol-2-yl)methyl]-N-[(4-methoxyphenyl)methyl]-2-(morpholin-4-yl)pyrazolo[1,5-a][1,3,5]triazin-4-amine

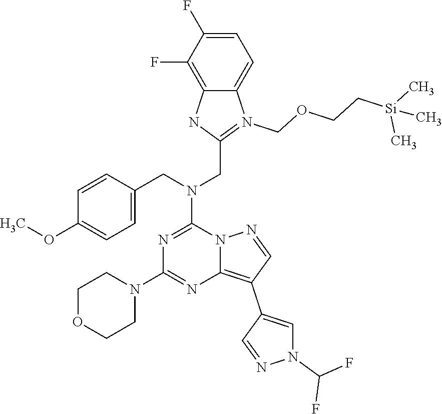

To a solution of 8-[1-(difluoromethyl)-1H-pyrazol-4-yl]-N-[(4-methoxyphenyl)methyl]-2-(morpholin-4-yl)pyrazolo[1,5-a][1,3,5]triazin-4-amine (Intermediate 259, 295 mg, 0.36 mmol), 2-(chloromethyl)-4,5-difluoro-1-{[2-(trimethylsilyl)ethoxy]methyl}-1H-benzimidazole (Intermediate 158, 206 mg, 0.612 mmol) in N,N-dimethylformamide (6.0 mL) was added potassium carbonate (149 mg, 1.08 mmol) at room temperature. After stirring at 50° C. for 16 hours, the reaction mixture was diluted with water and extracted with ethyl acetate. The combined organic layers were washed with water and brine, dried over anhydrous sodium sulfate and concentrated to give a residue. The residue was purified by preparative TLC (petroleum ether: ethyl acetate=1:1) to give 8-[1-(difluoromethyl)-1H-pyrazol-4-yl]-N-[(4,5-difluoro-1-{[2-(trimethylsilyl)ethoxy]methyl}-1H-benzimidazol-2-yl)methyl]-N-(4-methoxybenzyl)-2-(morpholin-4-yl)pyrazolo[1,5-a][1,3,5]triazin-4-amine (160 mg, 94% purity, 55% yield) as yellow oil.

LC-MS (Method C): Rₜ=1.17 min; MS (ESIpos): m/z=753.4 [M+H]⁺.

### Intermediate 262

### N-[(5,6-dichloro-1-{[2-(trimethylsilyl)ethoxy]methyl}-1H-benzimidazol-2-yl)methyl]-8-[1-(difluoromethyl)-1H-pyrazol-4-yl]-N-[(4-methoxyphenyl)methyl]-2-(morpholin-4-yl)pyrazolo[1,5-a][1,3,5]triazin-4-amine

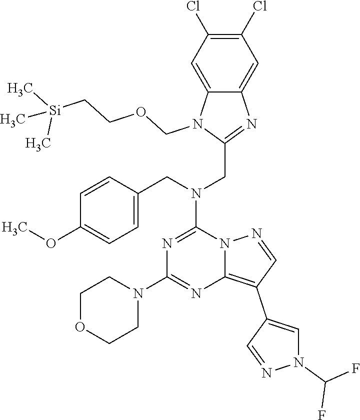 [SMILES: COc1ccc(CN(Cc2nc3cc(Cl)c(Cl)cc3n2COCC[Si](C)(C)C)c2nc(N3CCOCC3)nc3c(-c4cnn(C(F)F)c4)cnn23)cc1]

To a solution of 8-[1-(difluoromethyl)-1H-pyrazol-4-yl]-N-[(4-methoxyphenyl)methyl]-2-(morpholin-4-yl)pyrazolo[1,5-a][1,3,5]triazin-4-amine (Intermediate 259, 290 mg, 56% purity, 0.354 mmol), 5,6-dichloro-2-(chloromethyl)-1-{[2-(trimethylsilyl)ethoxy]methyl}-1H-benzimidazole (Intermediate 250, 181 mg, 86% purity, 0.425 mmol) in N,N-dimethylformamide (3.3 mL) was added potassium carbonate (97.8 mg, 0.708 mmol) at room temperature. After stirring at 50° C. for 16 hours, the reaction mixture was diluted with water and extracted with ethyl acetate. The combined organic layers were washed with water and brine, dried over anhydrous sodium sulfate and concentrated to give a residue. The residue was purified by preparative TLC (petroleum ether: ethyl acetate=1:1) to give N-[(5,6-dichloro-1-{[2-(trimethylsilyl)ethoxy]methyl}-1H-benzimidazol-2-yl)methyl]-8-[1-(difluoromethyl)-1H-pyrazol-4-yl]-N-(4-methoxybenzyl)-2-(morpholin-4-yl)pyrazolo[1,5-a][1,3,5]triazin-4-amine (200 mg, 62% purity, 45% yield) as yellow oil.

LC-MS (Method C): Rₜ=1.21 min; MS (ESIpos): m/z=785.4 [M+H]⁺.

### Intermediate 263

### N,N-bis[(4-methoxyphenyl)methyl]-2-(morpholin-4-yl)-8-(1,3-thiazol-5-yl)pyrazolo[1,5-a][1,3,5]triazin-4-amine

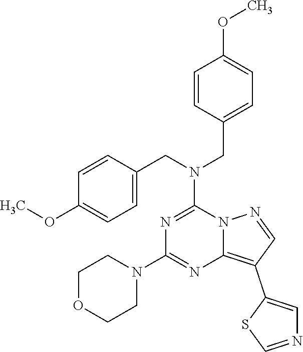 [SMILES: COc1ccc(CN(Cc2ccc(OC)cc2)c2nc(N3CCOCC3)nc3c(-c4cncs4)cnn23)cc1]

To a solution of [4-{bis[(4-methoxyphenyl)methyl]amino}-2-(morpholin-4-yl)pyrazolo[1,5-a][1,3,5]triazin-8-yl]boronic acid (Intermediate 242, 4.00 g, 7.93 mmol) and 5-bromo-1,3-thiazole (1.43 g, 8.72 mmol) in tetrahydrofuran (60 mL) were added methanesulfonato(2-dicyclohexylphosphino-2′,4′,6′-tri-i-propyl-1,1′-biphenyl)(2′-amino-1,1′-biphenyl-2-yl)palladium(II) (336 mg, 0.40 mmol) and potassium phosphate (12 mL, 24 mmol, 2.0 M in water) at room temperature. After stirring at 60° C. for 16 hours, the reaction mixture was diluted with water, extracted with ethyl acetate. The organic phase was washed with brine, dried over anhydrous sodium sulfate, concentrated to give a residue. The residue was purified by flash column chromatography (petroleum ether: ethyl acetate=20:1 to 1:1) to give N,N-bis(4-methoxybenzyl)-2-(morpholin-4-yl)-8-(1,3-thiazol-5-yl)pyrazolo[1,5-a][1,3,5]triazin-4-amine (710 mg, 96% purity, 16% yield) as a brown solid.

LC-MS (Method D): Rₜ=0.94 min; MS (ESIpos): m/z=544.2 [M+H]⁺.

### Intermediate 264

### N-[(4-methoxyphenyl)methyl]-2-(morpholin-4-yl)-8-(1,3-thiazol-5-yl)pyrazolo[1,5-a][1,3,5]triazin-4-amine

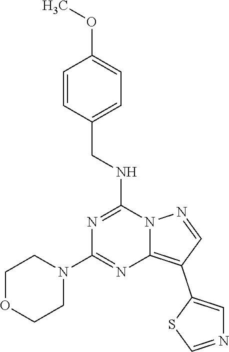 [SMILES: COc1ccc(CNc2nc(N3CCOCC3)nc3c(-c4cncs4)cnn23)cc1]

A solution of N,N-bis[(4-methoxyphenyl)methyl]-2-(morpholin-4-yl)-8-(1,3-thiazol-5-yl)pyrazolo[1,5-a][1,3,5]triazin-4-amine (Intermediate 263, 1.08 g, 96% purity, 1.91 mmol) in trifluoroacetic acid (15 mL) was stirred at room temperature for 2 hours. The reaction mixture was adjusted to pH=7 with cooled saturated sodium bicarbonate aqueous solution, and extracted with ethyl acetate. The organic phase was washed with brine, dried over anhydrous sodium sulfate, concentrated to give a residue. The residue was purified by flash column chromatography (petroleum ether: ethyl acetate=20:1 to 1:1) to give N-(4-methoxybenzyl)-2-(morpholin-4-yl)-8-(1,3-thiazol-5-yl)pyrazolo[1,5-a][1,3,5]triazin-4-amine (830 mg, 83% purity, 85% yield) as a brown solid.

LC-MS (Method C): Rₜ=0.83 min; MS (ESIpos): m/z=423.3 [M+H]⁺.

### Intermediate 265

### N-[(5,6-dichloro-1-{[2-(trimethylsilyl)ethoxy]methyl}-1H-benzimidazol-2-yl)methyl]-N-[(4-methoxyphenyl)methyl]-2-(morpholin-4-yl)-8-(1,3-thiazol-4-yl)pyrazolo[1,5-a][1,3,5]triazin-4-amine

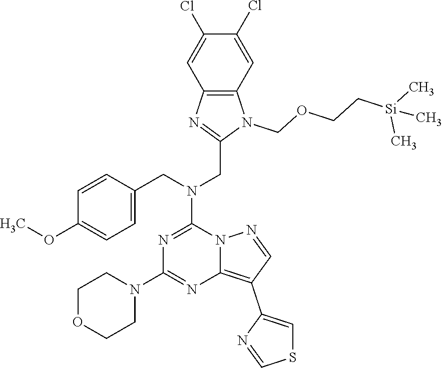

To a solution of N-[(4-methoxyphenyl)methyl]-2-(morpholin-4-yl)-8-(1,3-thiazol-4-yl)pyrazolo[1,5-a][1,3,5]triazin-4-amine (Intermediate 264, 200 mg, 79% purity, 0.37 mmol) in N,N-dimethylformamide (4.0 mL) were added potassium carbonate (155 mg, 1.12 mmol) and 5,6-dichloro-2-(chloromethyl)-1-{[2-(trimethylsilyl)ethoxy]methyl}-1H-benzimidazole (Intermediate 250, 190 mg, 86% purity, 0.50 mmol) at room temperature. After stirring at 50° C. for 16 hours, the reaction mixture was diluted with water and extracted with ethyl acetate. The combined organic layers were washed with water and brine, dried over anhydrous sodium sulfate and concentrated to give the residue. The residue was purified by preparative TLC (petroleum ether: ethyl acetate=2:1) to give N-[(5,6-dichloro-1-{[2-(trimethylsilyl)ethoxy] methyl}-1H-benzimidazol-2-yl)methyl]-N-(4-methoxybenzyl)-2-(morpholin-4-yl)-8-(1,3-thiazol-4-yl)pyrazolo[1,5-a][1,3,5]triazin-4-amine (250 mg, 95% purity, 85% yield) as brown oil.

LC-MS (Method C): Rₜ=1.08 min; MS (ESIpos): m/z=752.1 [M+H]⁺.

### Intermediate 266

### 1-[4-{bis[(4-methoxyphenyl)methyl]amino}-2-(morpholin-4-yl)pyrazolo[1,5-a][1,3,5]triazin-8-yl]-2,2-difluoroethan-1-one

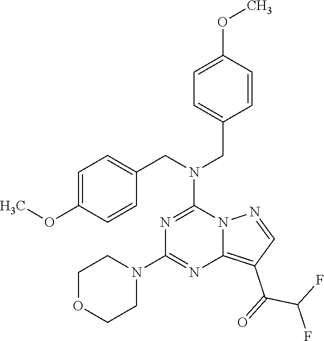 [SMILES: COc1ccc(CN(Cc2ccc(OC)cc2)c2nc(N3CCOCC3)nc3c(C(=O)C(F)F)cnn23)cc1]

To a solution of 8-iodo-N,N-bis[(4-methoxyphenyl)methyl]-2-(morpholin-4-yl)pyrazolo[1,5-a][1,3,5]triazin-4-amine (Intermediate 128, 500 mg, 98% purity, 0.84 mmol) in tetrahydrofuran (9.8 mL) was added isopropylmagnesium chloride (0.84 mL, 1.7 mmol, 2.0 M in tetrahydrofuran) at 0° C. After stirring at room temperature for 1 hour, 2,2-difluoro-N-methoxy-N-methylacetamide (291 mg, 2.09 mmol) was added dropwise to the above mixture. The solution was stirred at room temperature for another 16 hours. The reaction mixture was quenched with saturated ammonium chloride aqueous solution, diluted with water and extracted with ethyl acetate. The combined organic layers were washed with water and brine, dried over anhydrous sodium sulfate and concentrated to give a residue. The residue was purified by silica gel column chromatograhy (1000 mesh petroleum ether: ethyl acetate=20:1, then 10:1, then 5:1, then 3:1, then 2:1, then 1:1) to give 1-{4-[bis(4-methoxybenzyl)amino]-2-(morpholin-4-yl)pyrazolo[1,5-a][1,3,5]triazin-8-yl}-2,2-difluoroethanone (240 mg, 88% purity, 47% yield) as a brown solid.

LC-MS (Method C): Rₜ=1.04 min; MS (ESIpos): m/z=539.1 [M+H]⁺.

### Intermediate 267

### 8-(2,2-difluoroethyl)-N-[(4-methoxyphenyl)methyl]-2-(morpholin-4-yl)pyrazolo[1,5-a][1,3,5]triazin-4-amine

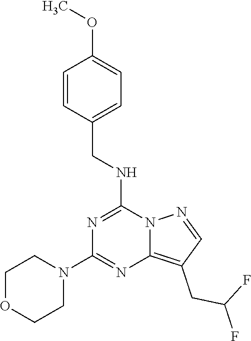 [SMILES: COc1ccc(CNc2nc(N3CCOCC3)nc3c(CC(F)F)cnn23)cc1]

A solution of 1-[4-{bis[(4-methoxyphenyl)methyl]amino}-2-(morpholin-4-yl)pyrazolo[1,5-a][1,3,5]triazin-8-yl]-2,2-difluoroethan-1-one (Intermediate 266, 2.20 g, 77% purity, 3.15 mmol) in a mixture solvent of trifluoroacetic acid (30 mL) and triethylsilane (15 mL) was stirred at 80° C. for 3 hours. The reaction mixture was concentrated, diluted with water. After adjusted to pH=7 by sodium bicarbonate, the solution was extracted with ethyl acetate. The combined organic layers were washed with water and brine, dried over anhydrous sodium sulfate and concentrated to give a residue. The residue was purified by flash column chromatography (300-400 mesh, petroleum ether: ethyl acetate=20:1 to 1:1) to give 8-(2,2-difluoroethyl)-N-(4-methoxybenzyl)-2-(morpholin-4-yl)pyrazolo[1,5-a][1,3,5]triazin-4-amine (580 mg, 83% purity, 38% yield) as a brown solid.

LC-MS (Method C): Rₜ=0.86 min; MS (ESIpos): m/z=405.0 [M+H]⁺.

### Intermediate 268

### 8-(2,2-difluoroethyl)-N-[(4,5-difluoro-1-{[2-(trimethylsilyl)ethoxy]methyl}-1H-benzimidazol-2-yl)methyl]-N-[(4-methoxyphenyl)methyl]-2-(morpholin-4-yl)pyrazolo[1,5-a][1,3,5]triazin-4-amine

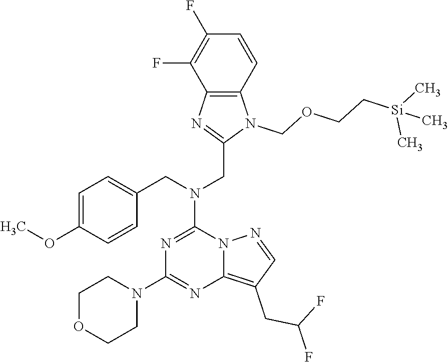

To a solution of 8-(2,2-difluoroethyl)-N-[(4-methoxyphenyl)methyl]-2-(morpholin-4-yl)pyrazolo [1,5-a][1,3,5]triazin-4-amine (Intermediate 267, 160 mg, 83% purity, 0.33 mmol) in N,N-dimethylformamide (4.0 mL) were added potassium carbonate (136 mg, 0.99 mmol) and 2-(chloromethyl)-4,5-difluoro-1-{[2-(trimethylsilyl)ethoxy]methyl}-1H-benzimidazole (Intermediate 158, 157 mg, 84% purity, 0.39 mmol) at room temperature. After stirring at 60° C. for 16 hours, the reaction mixture was diluted with water and extracted with ethyl acetate. The combined organic layers were washed with water and brine, dried over anhydrous sodium sulfate and concentrated to give a residue. The residue was purified by preparative TLC (petroleum ether: ethyl acetate=2:1) to give 8-(2,2-difluoroethyl)-N-[(4,5-difluoro-1-{[2-(trimethylsilyl)ethoxy] methyl}-1H-benzimidazol-2-yl)methyl]-N-(4-methoxybenzyl)-2-(morpholin-4-yl)pyrazolo[1,5-a][1,3,5]triazin-4-amine (180 mg, 99% purity, 77% yield) as yellow oil.

LC-MS (Method C): Rₜ=1.16 min; MS (ESIpos): m/z=701.3 [M+H]⁺.

### Intermediate 269

### N-[(4,5-difluoro-1-{[2-(trimethylsilyl)ethoxy]methyl}-1H-benzimidazol-2-yl)methyl]-2-(morpholin-4-yl)-8-(trifluoromethyl)pyrazolo[1,5-a][1,3,5]triazin-4-amine

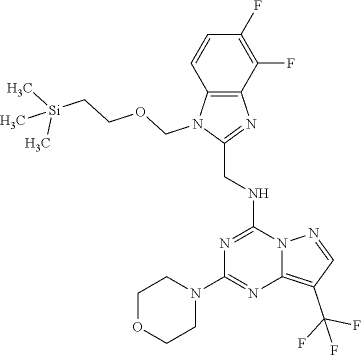 [SMILES: C[Si](C)(C)CCOCn1c(CNc2nc(N3CCOCC3)nc3c(C(F)(F)F)cnn23)nc2c(F)c(F)ccc21]

The reaction was performed in two separate batches. To a solution of 2-(morpholin-4-yl)-8-(trifluoromethyl)pyrazolo[1,5-a][1,3,5]triazin-4-amine (Intermediate 131, 30/100 mg, 84% purity, 87.4 μmol/0.29 mmol) in N,N-dimethylformamide (2.0/5.0 mL) was added sodium hydride (4.2/17.5 mg, 105 μmol/0.44 mmol, 60% purity in mineral oil) at room temperature. After stirring at 60° C. for 15 minutes, 2-(chloromethyl)-4,5-difluoro-1-{[2-(trimethylsilyl)ethoxy] methyl}-1H-benzimidazole (Intermediate 158, 29.1/126 mg, 87.4 μmol/0.38 mmol) was added in one portion. The reaction mixture was stirred at room temperature for 16 hours. The reaction mixtures were combined, with water and extracted with ethyl acetate. The organic phase was washed with water and brine, dried over anhydrous sodium sulfate and filtered. The filtrate was concentrated to give N-[(4,5-difluoro-1-{[2-(trimethylsilyl)ethoxy] methyl}-1H-benzimidazol-2-yl)methyl]-2-(morpholin-4-yl)-8-(trifluoromethyl)pyrazolo[1,5-a][1,3,5]triazin-4-amine (150 mg, 78% purity, 69% yield) as yellow oil.

LC-MS (Method C): Rₜ=1.11 min; MS (ESIpos): m/z=585.3 [M+H]⁺.

### Intermediate 270

### methyl 3-bromo-1-(tetrahydro-2H-pyran-2-yl)-1H-1,2,4-triazole-5-carboxylate

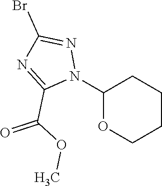 [SMILES: COC(=O)c1nc(Br)nn1C1CCCCO1]

To a solution of methyl 3-bromo-1H-1,2,4-triazole-5-carboxylate (3.34 g, 16.2 mmol) and 3,4-dihydro-2H-pyran (4.09 g, 48.6 mmol) in tetrahydrofuran (60 mL) was added 4- toluenesulfonic acid (279 mg, 1.62 mmol) at room temperature. After refluxing for 4 hours, the reaction mixture was concentrated, diluted with water and extracted with ethyl acetate. The combined organic layers were washed with water and brine, dried over anhydrous sodium sulfate and concentrated to give methyl 3-bromo-1-(tetrahydro-2H-pyran-2-yl)-1H-1,2,4-triazole-5-carboxylate (4.00 g, 80% purity, 68% yield) as brown oil.

### Intermediate 271

### methyl 3-phenyl-1-(tetrahydro-2H-pyran-2-yl)-1H-1,2,4-triazole-5-carboxylate

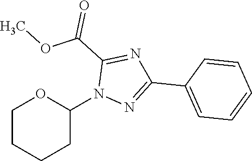 [SMILES: COC(=O)c1nc(-c2ccccc2)nn1C1CCCCO1]

To a solution of methyl 3-bromo-1-(tetrahydro-2H-pyran-2-yl)-1H-1,2,4-triazole-5-carboxylate (Intermediate 270, 4.08 g, 80% purity, 11.3 mmol) and phenylboronic acid (2.74 g, 22.5 mmol) in tetrahydrofuran (80 mL) were added methanesulfonato(2-dicyclohexylphosphino-2′,4′,6′-tri-i-propyl-1,1′-biphenyl)(2′-amino-1,1′-biphenyl-2-yl)palladium(II) (952 mg, 1.13 mmol) and potassium phosphate (15 mL, 1.5 M, 23 mmol) at in one portion room temperature. The reaction mixture was stirred at 60° C. for 16 hours under nitrogen atmosphere. The solution was concentrated, diluted with water and extracted with ethyl acetate. The combined organic layers were washed with water and brine, dried over anhydrous sodium sulfate and concentrated to give a crude product. The crude product was purified by silica gel column chromatography (200-300 mesh, petroleum ether: ethyl acetate=10:1, then 3:1, then 1:1) to give methyl 3-phenyl-1-(tetrahydro-2H-pyran-2-yl)-1H-1,2,4-triazole-5-carboxylate (3.50 g, 88% purity, 95% yield) as a yellow solid.

¹H NMR (400 MHz, DMSO-d₆) 5 [ppm]=7.82-7.76 (m, 2H), 7.66-7.60 (m, 3H), 5.51 (dd, J=9.6, 2.4 Hz, 1H), 4.07-4.00 (m, 1H), 3.90 (s, 3H), 3.72-3.63 (m, 1H), 2.35-2.24 (m, 1H), 2.03-1.89 (m, 2H), 1.73-1.51 (m, 3H).

### Intermediate 272

### [3-phenyl-1-(tetrahydro-2H-pyran-2-yl)-1H-1,2,4-triazol-5-yl]methanol

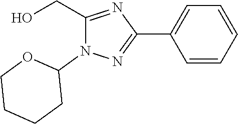 [SMILES: OCc1nc(-c2ccccc2)nn1C1CCCCO1]

To a solution of methyl 3-phenyl-1-(tetrahydro-2H-pyran-2-yl)-1H-1,2,4-triazole-5-carboxylate (Intermediate 271, 3.50 g, 88% purity, 10.7 mmol) in tetrahydrofuran (60 mL) was added lithium aluminum hydride (529 mg, 13.9 mmol) at 0° C. After stirring at 0° C. for 2 hours, the reaction was diluted with water and extracted with ethyl acetate. The combined organic layers were washed with water and brine, dried over anhydrous sodium sulfate and concentrated to give a crude product. The crude product was purified by silica gel column chromatography (200-300 mesh, petroleum ether: ethyl acetate=10:1, then 3:1, then 2:1) to give [3-phenyl-1-(tetrahydro-2H-pyran-2-yl)-1H-1,2,4-triazol-5-yl]methanol (1.45 g, 89% purity, 46% yield) as a white solid.

¹H NMR (400 MHz, DMSO-d₆) 5=7.73 (dd, J=7.2, 3.6 Hz, 2H), 7.58 (t, J=4.0 Hz, 3H), 5.34 (dd, J=9.6, 2.0 Hz, 2H), 4.48 (d, J=6.0 Hz, 2H), 4.02 (d, J=11.6 Hz, 1H), 3.66-3.60 (m, 1H), 2.35-2.25 (m, 1H), 1.99-1.91 (m, 1H), 1.90-1.82 (m, 1H), 1.67-1.50 (m, 3H).

### Intermediate 273

### [3-phenyl-1-(tetrahydro-2H-pyran-2-yl)-1H-1,2,4-triazol-5-yl]methyl methanesulfonate

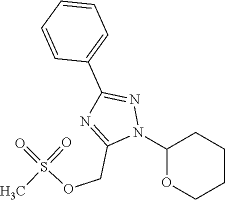 [SMILES: CS(=O)(=O)OCc1nc(-c2ccccc2)nn1C1CCCCO1]

To a solution of [3-phenyl-1-(tetrahydro-2H-pyran-2-yl)-1H-1,2,4-triazol-5-yl]methanol (Intermediate 272, 300 mg, 89% purity, 1.03 mmol) in dichloromethane (6.0 mL) were added triethylamine (0.3 mL, 2.1 mmol) and methanesulfonic anhydride (269 mg, 1.54 mmol) at 0° C. After stirring at room temperature for 3 hours, the reaction mixture was poured into ice-water and extracted with dichloromethane. The combined organic layers were washed with water and brine, dried over anhydrous sodium sulfate and concentrated to give [3-phenyl-1-(tetrahydro-2H-pyran-2-yl)-1H-1,2,4-triazol-5-yl]methyl methanesulfonate (300 mg, 80% purity, 69% yield) as yellow oil.

¹H NMR (400 MHz, CDCl₃) δ [ppm]=7.79-7.72 (m, 2H), 7.59-7.50 (m, 3H), 5.34 (dd, J=10, 2.8 Hz, 1H), 5.30 (s, 2H), 4.18 (dt, J=11.2, 2.0 Hz, 1H), 3.69 (td, J=11.2, 2.4 Hz, 1H), 3.16 (s, 3H), 2.55-2.41 (m, 1H), 2.17-2.07 (m, 1H), 1.93-1.84 (m, 1H), 1.83-1.75 (m, 1H), 1.67-1.57 (m, 2H).

### Intermediate 274

### 2-(morpholin-4-yl)-N-{[3-phenyl-1-(tetrahydro-2H-pyran-2-yl)-1H-1,2,4-triazol-5-yl]methyl}-8-(trifluoromethyl)pyrazolo[1,5-a][1,3,5]triazin-4-amine

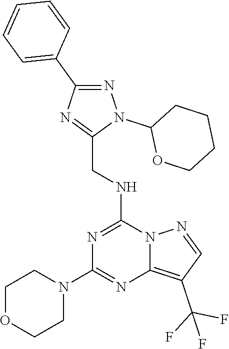 [SMILES: FC(F)(F)c1cnn2c(NCc3nc(-c4ccccc4)nn3C3CCCCO3)nc(N3CCOCC3)nc12]

The reaction was performed in two separate batches. To a solution of 2-(morpholin-4-yl)-8-(trifluoromethyl)pyrazolo[1,5-a][1,3,5]triazin-4-amine (Intermediate 131, 40/200 mg, 138.8 μmol/0.70 mmol) in N,N-dimethylformamide (2.0/10 mL) was added sodium hydride (8.3/41.6 mg, 208.1 μmol/1.04 mmol, 60% purity) at 0° C. After stirring at 50° C. for 10 minutes, [3-phenyl-1-(tetrahydro-2H-pyran-2-yl)-1H-1,2,4-triazol-5-yl]methyl methanesulfonate (Intermediate 273, 46.8/234 mg, 138.8 μmol/0.70 mmol) was added to the above mixture at room temperature. The mixture was stirred at 30° C. for 16 hours. The reaction mixtures were combined, diluted with water and extracted with ethyl acetate. The combined organic layers were washed with water and brine, dried over anhydrous sodium sulfate and concentrated to give a crude product. The crude product was purified by preparative TLC (methanol: dichloromethane=1/10) to give 2-(morpholin-4-yl)-N-{[3-phenyl-1-(tetrahydro-2H-pyran-2-yl)-1H-1,2,4-triazol-5-yl]methyl}-8-(trifluoromethyl)pyrazolo[1,5-a][1,3,5]triazin-4-amine (300 mg, 80% purity, 65% yield) as a brown solid.

LC-MS (Method C): Rₜ=0.94 min; MS (ESIpos): m/z=530.2 [M+H]⁺.

### Intermediate 275

### benzyl 4-(4-{bis[(4-methoxyphenyl)methyl]amino}-8-ethenylpyrazolo[1,5-a][1,3,5]triazin-2-yl)piperazine-1-carboxylate

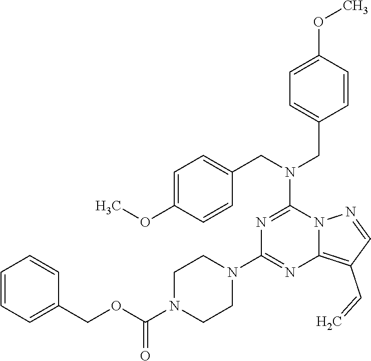 [SMILES: C=Cc1cnn2c(N(Cc3ccc(OC)cc3)Cc3ccc(OC)cc3)nc(N3CCN(C(=O)OCc4ccccc4)CC3)nc12]

To a solution of benzyl 4-(4-{bis[(4-methoxyphenyl)methyl]amino}-8-iodopyrazolo[1,5-a][1,3,5]triazin-2-yl)piperazine-1-carboxylate (Intermediate 235, 3.00 g, 4.17 mmol) and potassium trifluoro(vinyl)borate (2.23 g, 16.7 mmol) in tetrahydrofuran (60 mL) were added methanesulfonato(2-dicyclohexylphosphino-2′,4′,6′-tri-i-propyl-1,1′-biphenyl)(2′-amino-1,1′-biphenyl-2-yl)palladium(II) (353 mg, 0.42 mmol) and potassium phosphate (8.3 mL, 13 mmol, 1.5 M in water) at room temperature. After stirring at 80° C. for 16 hours, the solution was diluted with water and extracted with ethyl acetate. The combined organic layers were washed with water and brine, dried over anhydrous sodium sulfate and concentrated to give a crude product. The crude product was purified by neutral aluminium oxide column chromatography (petroleum ether: ethyl acetate=10:1 then 3:1) to give benzyl 4-{4-[bis(4-methoxybenzyl)amino]-8-vinylpyrazolo[1,5-a][1,3,5]triazin-2-yl}piperazine-1-carboxylate (1.40 g, 37% yield) as yellow oil.

LC-MS (Method C): Rₜ=1.10 min; MS (ESIpos): m/z=620.3 [M+H]⁺.

### Intermediate 276

### benzyl 4-{4-[bis(4-methoxybenzyl)amino]-8-(2,2-difluorocyclopropyl)pyrazolo[1,5-a][1,3,5]triazin-2-yl}piperazine-1-carboxylate

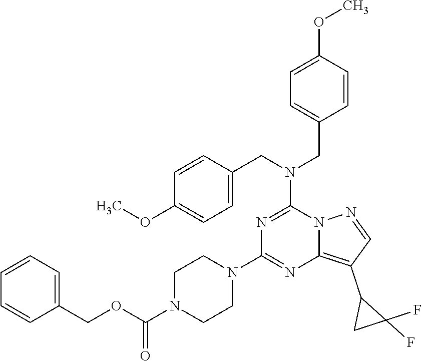 [SMILES: COc1ccc(CN(Cc2ccc(OC)cc2)c2nc(N3CCN(C(=O)OCc4ccccc4)CC3)nc3c(C4CC4(F)F)cnn23)cc1]

A solution of benzyl 4-(4-{bis[(4-methoxyphenyl)methyl]amino}-8-ethenylpyrazolo[1,5-a][1,3,5]triazin-2-yl)piperazine-1-carboxylate (Intermediate 275, 1.40 g, 1.54 mmol), trimethyl (trifluoromethyl)silane (2.18 g, 15.4 mmol) and sodium iodate (46.1 mg, 0.31 mmol) in tetrahydrofuran (20 mL) was refluxed for 16 hours under nitrogen atmosphere. The solution was diluted with water and extracted with ethyl acetate. The combined organic layers were washed with water and brine, dried over anhydrous sodium sulfate and concentrated to give a crude product. The crude product was purified by silica gel column chromatography (100-200 mesh, petroleum ether: ethyl acetate=5:1, then 2:1) to give benzyl 4-{4-[bis(4-methoxybenzyl)amino]-8-(2,2-difluorocyclopropyl)pyrazolo[1,5-a][1,3,5]triazin-2-yl}piperazine-1-carboxylate (1.00 g, 87% yield) as a yellow solid.

LC-MS (Method D): Rₜ=1.06 min; MS (ESIpos): m/z=670.2 [M+H]⁺.

### Intermediate 277

### 8-(2,2-difluorocyclopropyl)-N-(4-methoxybenzyl)-2-(piperazin-1-yl)pyrazolo[1,5-a][1,3,5]triazin-4-amine

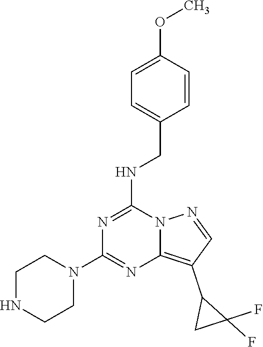 [SMILES: COc1ccc(CNc2nc(N3CCNCC3)nc3c(C4CC4(F)F)cnn23)cc1]

A solution of benzyl 4-{4-[bis(4-methoxybenzyl)amino]-8-(2,2-difluorocyclopropyl)pyrazolo[1,5-a][1,3,5]triazin-2-yl}piperazine-1-carboxylate (Intermediate 276, 1.30 g, 1.36 mmol) in trifluoroacetic acid (10 mL) was stirred at 60° C. for 3 hours. The solution was concentrated to give 8-(2,2-difluorocyclopropyl)-N-(4-methoxybenzyl)-2-(piperazin-1-yl)pyrazolo[1,5-a][1,3,5]triazin-4-amine (800 mg, 99% yield) as brown oil.

LC-MS (Method C): Rₜ=0.82 min; MS (ESIpos): m/z=416.3 [M+H]⁺.

### Intermediate 278

### benzyl 4-{8-(2,2-difluorocyclopropyl)-4-[(4-methoxybenzyl)amino]pyrazolo[1,5-a][1,3,5]triazin-2-yl}piperazine-1-carboxylate

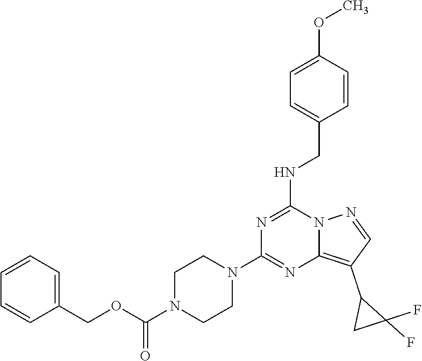 [SMILES: COc1ccc(CNc2nc(N3CCN(C(=O)OCc4ccccc4)CC3)nc3c(C4CC4(F)F)cnn23)cc1]

To a solution of 8-(2,2-difluorocyclopropyl)-N-(4-methoxybenzyl)-2-(piperazin-1-yl)pyrazolo[1,5-a][1,3,5]triazin-4-amine (Intermediate 277, 800 mg, 1.35 mmol) and sodium bicarbonate (340 mg, 4.04 mmol) in a mixed solvent of tetrahydrofuran (10 mL) and water (8.0 mL) was added dropwise benzyl chloroformate (0.29 mL, 2.0 mmol) at 0° C. The mixture was stirred at room temperature for 3 hours. The solution was diluted with water and extracted with ethyl acetate. The combined organic layers were washed with water and brine, dried over anhydrous sodium sulfate and concentrated to give a residue. The residue was purified by silica gel column chromatography (100-200 mesh, petroleum ether: ethyl acetate=5:1, then 3:1) to give benzyl 4-{8-(2,2-difluorocyclopropyl)-4-[(4-methoxybenzyl)amino]pyrazolo[1,5-a][1,3,5]triazin-2-yl}piperazine-1-carboxylate (1.00 g) as a yellow solid.

LC-MS (Method C): Rₜ=1.08 min; MS (ESIpos): m/z=550.4 [M+H]⁺.

### Intermediate 279

### benzyl 4-[8-(2,2-difluorocyclopropyl)-4-{(4-methoxybenzyl)[(1-{[2-(trimethylsilyl)ethoxy]methyl}-1H-benzimidazol-2-yl)methyl]amino}pyrazolo[1,5-a][1,3,5]triazin-2-yl]piperazine-1-carboxylate

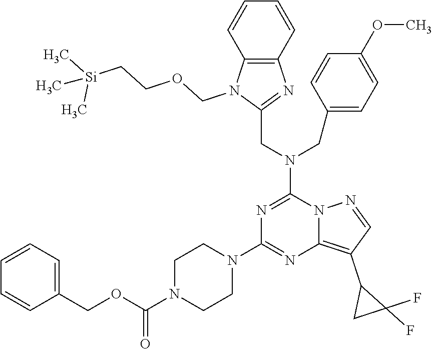 [SMILES: COc1ccc(CN(Cc2nc3ccccc3n2COCC[Si](C)(C)C)c2nc(N3CCN(C(=O)OCc4ccccc4)CC3)nc3c(C4CC4(F)F)cnn23)cc1]

To a solution of benzyl 4-{8-(2,2-difluorocyclopropyl)-4-[(4-methoxybenzyl)amino]pyrazolo[1,5-a][1,3,5]triazin-2-yl}piperazine-1-carboxylate (Intermediate 278, 65.5 mg, 1.64 mmol) in N,N-Dimethylformamide (5 mL) was added sodium hydride (65.0 mg, 1.64 mmol, 60% purity in mineral oil) at room temperature. After stirring at 60° C. for 15 minutes, 2-(chloromethyl)-1-{[2-(trimethylsilyl)ethoxy]methyl}-1H-benzimidazole (Intermediate 130, 324 mg, 1.09 mmol) was added to the above mixture. The mixture was stirred at room temperature for 16 hours. The solution was diluted with water and extracted with ethyl acetate. The combined organic layers were washed with water and brine, dried over anhydrous sodium sulfate and concentrated to give a crude product. The crude product was purified by preparative TLC (petroleum ether: ethyl acetate=3:1) to give benzyl 4-[8-(2,2-difluorocyclopropyl)-4-{(4-methoxybenzyl)[(1-{[2-(trimethylsilyl)ethoxy]methyl}-1H-benzimidazol-2-yl)methyl]amino}pyrazolo[1,5-a][1,3,5]triazin-2-yl]piperazine-1-carboxylate (200 mg, 36% yield) as yellow oil.

LC-MS (Method D): Rₜ=1.01 min; MS (ESIpos): m/z=810.3 [M+H]⁺.

### Intermediate 280

### 8-(2,2-difluorocyclopropyl)-N-(4-methoxybenzyl)-2-(4-methylpiperazin-1-yl)-N-[(1-{[2-(trimethylsilyl)ethoxy]methyl}-1H-benzimidazol-2-yl)methyl]pyrazolo[1,5-a][1,3,5]triazin-4-amine

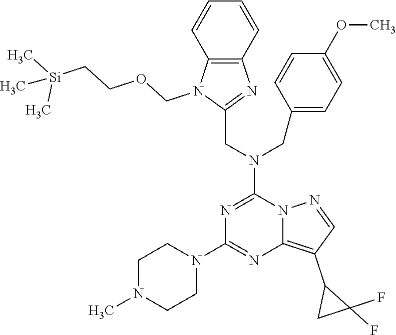 [SMILES: COc1ccc(CN(Cc2nc3ccccc3n2COCC[Si](C)(C)C)c2nc(N3CCN(C)CC3)nc3c(C4CC4(F)F)cnn23)cc1]

To a solution of benzyl 4-[8-(2,2-difluorocyclopropyl)-4-{(4-methoxybenzyl)[(1-{[2-(trimethylsilyl)ethoxy]methyl}-1H-benzimidazol-2-yl)methyl]amino}pyrazolo[1,5-a][1,3,5]triazin-2-yl]piperazine-1-carboxylate (Intermediate 279, 200 mg, 0.25 mmol) in methanol (5.0 mL) was added formaldehyde (0.3 mL) at 25° C. Palladium (20.0 mg, 10% on activated carbon) was added to the above mixture. The mixture was stirred at 25° C. for 16 hours under hydrogen atmosphere (15 psi). The reaction mixture was filtered, the filtrate was extracted with ethyl acetate. The organic phase was washed with brine, dried over anhydrous sodium sulfate and filtered, concentrated to give a crude product. The crude product was purified by TLC (dichloromethane:methanol=10:1) to give 8-(2,2-difluorocyclopropyl)-N-(4-methoxybenzyl)-2-(4-methylpiperazin-1-yl)-N-[(1-{[2-(trimethylsilyl)ethoxy]methyl}-1H-benzimidazol-2-yl)methyl]pyrazolo[1,5-a][1,3,5]triazin-4-amine (50.0 mg, 28% yield) as colorless oil.

LC-MS (Method C): Rₜ=0.94 min; MS (ESIpos): m/z=690.6 [M+H]⁺.

### Intermediate 281

### benzyl 4-[8-(2,2-difluorocyclopropyl)-4-{[(4,5-difluoro-1-{[2-(trimethylsilyl)ethoxy]methyl}-1H-benzimidazol-2-yl)methyl](4-methoxybenzyl)amino}pyrazolo[1,5-a][1,3,5]triazin-2-yl]piperazine-1-carboxylate

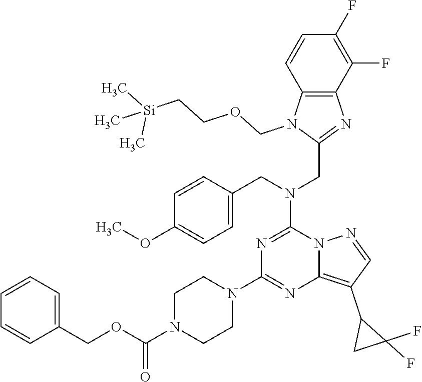 [SMILES: COc1ccc(CN(Cc2nc3c(F)c(F)ccc3n2COCC[Si](C)(C)C)c2nc(N3CCN(C(=O)OCc4ccccc4)CC3)nc3c(C4CC4(F)F)cnn23)cc1]

To a solution of benzyl 4-{8-(2,2-difluorocyclopropyl)-4-[(4-methoxybenzyl)amino]pyrazolo[1,5-a][1,3,5]triazin-2-yl}piperazine-1-carboxylate (Intermediate 278, 300 mg, 74% purity, 0.4 mmol) in N,N-dimethylformamide (5.0 mL) was added sodium hydride (48.1 mg, 1.20 mmol, 60% purity in mineral oil) at 25° C. After stirring at 60° C. for 15 minutes, 2-(chloromethyl)-4,5-difluoro-1-{[2-(trimethylsilyl)ethoxy]methyl}-1H-benzimidazole (Intermediate 158, 271 mg, 0.8 mmol) was added to the above mixture. The mixture was stirred at 25° C. for 16 hours. The reaction mixture was poured into water, extracted with ethyl acetate. The organic phase was washed with brine, dried over anhydrous sodium sulfate and filtered, concentrated to give a crude product. The crude product was purified by silica gel column chromatography (100-200 mesh, petroleum ether: ethyl acetate=10:1, then 5:1, then 3:1, then 2:1, then 1:1) to give benzyl 4-[8-(2,2-difluorocyclopropyl)-4-{[(4,5-difluoro-1-{[2-(trimethylsilyl)ethoxy]methyl}-1H-benzimidazol-2-yl)methyl](4-methoxybenzyl)amino}pyrazolo[1,5-a][1,3,5]triazin-2-yl]piperazine-1-carboxylate (240 mg, 53% purity, 19% yield) as yellow oil.

LC-MS (Method C): Rₜ=1.26 min; MS (ESIpos): m/z=846.5 [M+H]⁺.

### Intermediate 282

### N-((4,5-difluoro-1H-benzo[d]imidazol-2-yl)methyl)-8-(2,2-difluorocyclopropyl)-2-(piperazin-1-yl)pyrazolo[1,5-a][1,3,5]triazin-4-amine

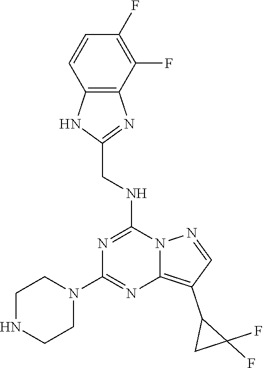 [SMILES: Fc1ccc2[nH]c(CNc3nc(N4CCNCC4)nc4c(C5CC5(F)F)cnn34)nc2c1F]

The reaction was performed in two separate batches. A solution of benzyl 4-[8-(2,2-difluorocyclopropyl)-4-{[(4,5-difluoro-1-{[2-(trimethylsilyl)ethoxy]methyl}-1H-benzimidazol-2-yl)methyl](4-methoxybenzyl)amino}pyrazolo[1,5-a][1,3,5]triazin-2-yl]piperazine-1-carboxylate (Intermediate 281, 30/180 mg, 53% purity, 35.5 μmol/0.110 mmol) in trifluoroacetic acid (0.5/3.0 mL) was stirred at 80° C. for 16 hours. The reactions were combined and concentrated to give N-((4,5-difluoro-1H-benzo[d]imidazol-2-yl)methyl)-8-(2,2-difluorocyclopropyl)-2-(piperazin-1-yl)pyrazolo[1,5-a][1,3,5]triazin-4-amine (70.0 mg, 69% purity, 93% yield) as black oil.

LC-MS (Method C): Rₜ=0.71 min; MS (ESIpos): m/z=462.2 [M+H]⁺.

### Intermediate 283

### methyl 3-bromo-1-[(4-methoxyphenyl)methyl]-1H-1,2,4-triazole-5-carboxylate

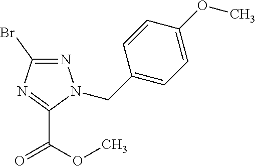 [SMILES: COC(=O)c1nc(Br)nn1Cc1ccc(OC)cc1]

To a solution of methyl 3-bromo-1H-1,2,4-triazole-5-carboxylate (9.50 g, 46.1 mmol) and N,N-diisopropylethylamine (11.9 g, 92.2 mmol) in tetrahydrofuran (120 mL) was added 1-(chloromethyl)-4-methoxybenzene (8.67 g, 55.3 mmol) at 25° C. After stirring at 50° C. for 16 hours, the reaction was diluted with water and extracted with ethyl acetate. The combined organic layers were washed with water and brine, dried over anhydrous sodium sulfate and concentrated to give a crude product. The crude product was purified by silica gel column chromatography (100-200 mesh, petroleum ether: ethyl acetate=5:1, then 3:1) to give methyl 3-bromo-1-(4-methoxybenzyl)-1H-1,2,4-triazole-5-carboxylate (9.40 g, 92% purity, 57% yield) as a white solid.

LC-MS (Method C): Rₜ=0.81 min; MS (ESIpos): m/z=326.0 [M+H]⁺.

### Intermediate 284

### methyl 1-(4-methoxybenzyl)-3-(2-methoxyphenyl)-1H-1,2,4-triazole-5-carboxylate

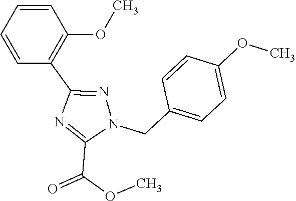 [SMILES: COC(=O)c1nc(-c2ccccc2OC)nn1Cc1ccc(OC)cc1]

To a mixture of methyl 3-bromo-1-[(4-methoxyphenyl)methyl]-1H-1,2,4-triazole-5-carboxylate (Intermediate 283, 4.00 g, 12.3 mmol) and (2-methoxyphenyl)boronic acid (3.73 g, 24.5 mmol) in tetrahydrofuran (80 mL) were added methanesulfonato(2-dicyclohexylphosphino-2′,4′,6′-tri-i-propyl-1,1′-biphenyl)(2′-amino-1,1′-biphenyl-2-yl)palladium(II) (1.04 g, 1.23 mmol) and potassium phosphate (16 mL, 1.5 M, 25 mmol) at room temperature in one portion. The reaction mixture was stirred at 60° C. for 16 hours under nitrogen atmosphere. The reaction mixture was diluted with water and extracted with ethyl acetate. The organic phase was washed with brine, dried over anhydrous sodium sulfate and filtered. The filtrate was concentrated and purified by silica gel column chromatography (100-200 mesh, petroleum ether: ethyl acetate=20:1, then 10:1, then 5:1) to give methyl 1-(4-methoxybenzyl)-3-(2-methoxyphenyl)-1H-1,2,4-triazole-5-carboxylate (2.60 g, 93% purity, 56% yield) as a brown solid.

LC-MS (Method C): Rₜ=0.82 min; MS (ESIpos): m/z=354.2 [M+H]⁺.

### Intermediate 285

### [1-(4-methoxybenzyl)-3-(2-methoxyphenyl)-1H-1,2,4-triazol-5-yl]methanol

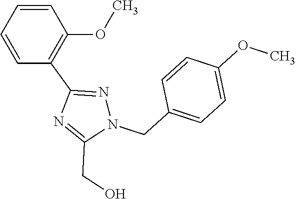 [SMILES: COc1ccc(Cn2nc(-c3ccccc3OC)nc2CO)cc1]

To a solution of methyl 1-(4-methoxybenzyl)-3-(2-methoxyphenyl)-1H-1,2,4-triazole-5-carboxylate (Intermediate 284, 2.60 g, 7.36 mmol) in tetrahydrofuran (50 mL) was added lithium aluminum hydride (558 mg, 14.7 mmol) slowly at 0° C. After stirring at 25° C. for 2 hours, the reaction mixture was added to saturated potassium sodium tartrate aqueous solution (20 mL), diluted with water and extracted with ethyl acetate. The combined organic layers were washed with water and brine, dried over anhydrous sodium sulfate and concentrated to give [1-(4-methoxybenzyl)-3-(2-methoxyphenyl)-1H-1,2,4-triazol-5-yl]methanol (2.06 g, 85% purity, 73% yield) as a yellow solid.

LC-MS (Method C): Rₜ=0.82 min; MS (ESIpos): m/z=326.3 [M+H]⁺.

### Intermediate 286

### 5-(chloromethyl)-3-(2-methoxyphenyl)-1-[(4-methoxyphenyl)methyl]-1H-1,2,4-triazole

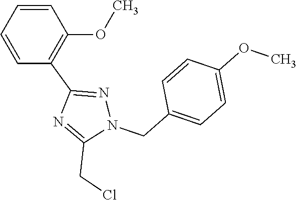 [SMILES: COc1ccc(Cn2nc(-c3ccccc3OC)nc2CCl)cc1]

To a solution of {3-(2-methoxyphenyl)-1-[(4-methoxyphenyl)methyl]-1H-1,2,4-triazol-5-yl}methanol (Intermediate 285, 2.06 g, 6.33 mmol) in a mixed solution of toluene (10 mL) and dichloromethane (5.0 mL) was added thionyl chloride (2.0 mL) at room temperature. After stirring at room temperature for 16 hours, the mixture was concentrated, diluted with water. Saturated sodium bicarbonate solution was adjusted to pH-7 and extracted with ethyl acetate. The combined organic layers were washed with water and brine, dried over anhydrous sodium sulfate and concentrated to give a crude product. The crude product was purified by silica gel column chromatography (100-200 mesh, petroleum ether: ethyl acetate=10:1, then 5:1) to give 5-(chloromethyl)-1-(4-methoxybenzyl)-3-(2-methoxyphenyl)-1H-1,2,4-triazole (1.56 g, 72% yield) as yellow oil.

LC-MS (Method C): Rₜ=0.91 min; MS (ESIpos): m/z=344.2 [M+H]⁺.

### Intermediate 287

### benzyl 4-{8-(2,2-difluorocyclopropyl)-4-[(4-methoxybenzyl){[1-(4-methoxybenzyl)-3-(2-methoxyphenyl)-1H-1,2,4-triazol-5-yl]methyl}amino]pyrazolo[1,5-a][1,3,5]triazin-2-yl}piperazine-1-carboxylate

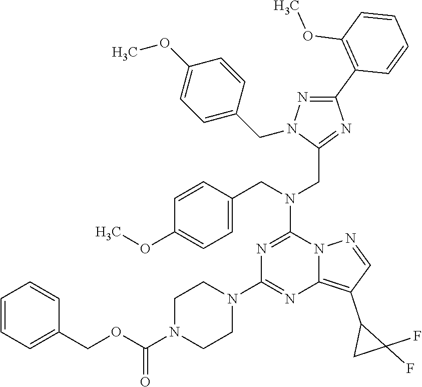 [SMILES: COc1ccc(CN(Cc2nc(-c3ccccc3OC)nn2Cc2ccc(OC)cc2)c2nc(N3CCN(C(=O)OCc4ccccc4)CC3)nc3c(C4CC4(F)F)cnn23)cc1]

The reaction was performed in two separate batches. To a solution of benzyl 4-{8-(2,2-difluorocyclopropyl)-4-[(4-methoxybenzyl)amino]pyrazolo[1,5-a][1,3,5]triazin-2-yl}piperazine-1-carboxylate (Intermediate 278, 50/150 mg, 0.09/0.27 mmol), 5-(chloromethyl)-3-(2-methoxyphenyl)-1-[(4-methoxyphenyl)methyl]-1H-1,2,4-triazole (Intermediate 286, 47/113 mg, 0.14/0.33 mmol) in N,N-dimethylformamide (1.0/2.0 mL) was added potassium carbonate (25.1/75.3 mg, 0.18/0.55 mmol) at room temperature. After stirring at 50° C. for 16 hours, the reaction mixtures were combined, diluted with water and extracted with ethyl acetate. The combined organic layers were washed with water and brine, dried over anhydrous sodium sulfate and concentrated to give a crude product. The crude product was purified by preparative-TLC (petroleum ether: ethyl acetate=1:1) to give benzyl 4-{8-(2,2-difluorocyclopropyl)-4-[(4-methoxybenzyl){[1-(4-methoxybenzyl)-3-(2-methoxyphenyl)-1H-1,2,4-triazol-5-yl]methyl}amino]pyrazolo[1,5-a][1,3,5]triazin-2-yl}piperazine-1-carboxylate (150 mg, 83% purity, 53% yield) as yellow oil.

LC-MS (Method C): Rₜ=1.17 min; MS (ESIpos): m/z=857.5 [M+H]⁺.

### Intermediate 288

### 8-(2,2-difluorocyclopropyl)-N-{[3-(2-methoxyphenyl)-1H-1,2,4-triazol-5-yl]methyl}-2-(piperazin-1-yl)pyrazolo[1,5-a][1,3,5]triazin-4-amine trifluoroacetate

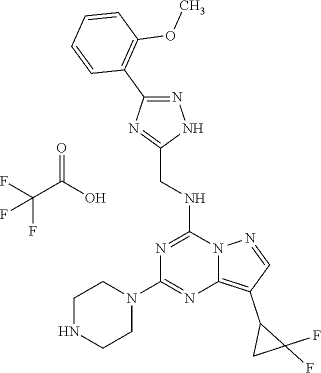 [SMILES: O=C(O)C(F)(F)F ; COc1ccccc1-c1n[nH]c(CNc2nc(N3CCNCC3)nc3c(C4CC4(F)F)cnn23)n1]

A solution of benzyl 4-{8-(2,2-difluorocyclopropyl)-4-[(4-methoxybenzyl){[1-(4-methoxybenzyl)-3-(2-methoxyphenyl)-1H-1,2,4-triazol-5-yl]methyl}amino]pyrazolo[1,5-a][1,3,5]triazin-2-yl}piperazine-1-carboxylate (Intermediate 287, 150 mg, 83% purity, 0.15 mmol) in trifluoroacetic acid (4.0 mL) was stirred at 80° C. for 5 hours. The mixture was concentrated to give 8-(2,2-difluorocyclopropyl)-N-{[3-(2-methoxyphenyl)-1H-1,2,4-triazol-5-yl]methyl}-2-(piperazin-1-yl)pyrazolo[1,5-a][1,3,5]triazin-4-amine trifluoroacetate (100 mg, 60% purity, 69% yield) as brown trifluoroacetic salt, used directly.

LC-MS (Method C): Rₜ=0.80 min; MS (ESIpos): m/z=483.4 [M+H]⁺.

### Intermediate 289

### 8-ethyl-N, N-bis[(4-methoxyphenyl)methyl]-2-(morpholin-4-yl)pyrazolo[1,5-a][1,3,5]triazin-4-amine

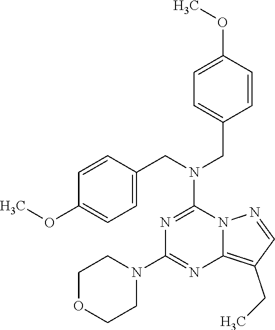 [SMILES: CCc1cnn2c(N(Cc3ccc(OC)cc3)Cc3ccc(OC)cc3)nc(N3CCOCC3)nc12]

A mixture of 8-ethenyl-N,N-bis[(4-methoxyphenyl)methyl]-2-(morpholin-4-yl)pyrazolo[1,5-a][1,3,5]triazin-4-amine (Intermediate 191, 1.20 g, 2.47 mmol) and palladium (600 mg, 10% on activated carbon) in tetrahydrofuran (10 mL) was stirred at 50° C. for 6 hours under hydrogen atmosphere (15 psi). The reaction mixture was filtered and the filtrate was concentrated to give 8-ethyl-N,N-bis(4-methoxybenzyl)-2-(morpholin-4-yl)pyrazolo[1,5-a][1,3,5]triazin-4-amine (1.20 g, 99% yield) as a white solid.

LC-MS (Method D): Rₜ=0.94 min; MS (ESIpos): m/z=489.2 [M+H]⁺.

### Intermediate 290

### 8-ethyl-N-[(4-methoxyphenyl)methyl]-2-(morpholin-4-yl)pyrazolo[1,5-a][1,3,5]triazin-4-amine

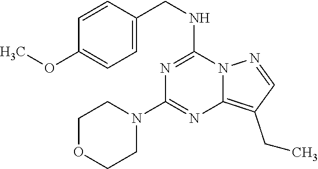 [SMILES: CCc1cnn2c(NCc3ccc(OC)cc3)nc(N3CCOCC3)nc12]

A solution of 8-ethyl-N,N-bis[(4-methoxyphenyl)methyl]-2-(morpholin-4-yl)pyrazolo[1,5-a][1,3,5]triazin-4-amine (Intermediate 289, 1.20 g, 2.46 mmol) in trifluoroacetic acid (10 mL, 130 mmol) was stirred at 80° C. for 3 hours. The reaction mixture was concentrated to give a residue. The residue was poured into water, adjusted to pH-7 by saturated sodium bicarbonate aqueous solution and extracted with ethyl acetate. The organic phase was washed with brine, dried over anhydrous sodium sulfate and filtered. The filtrate was concentrated and purified by flash chromatography (petroleum ether: ethyl acetate=10:1 to 1:1) to give 8-ethyl-N-(4-methoxybenzyl)-2-(morpholin-4-yl)pyrazolo[1,5-a][1,3,5]triazin-4-amine (850 mg, 94% yield) as a yellow solid.

LC-MS (Method D): Rₜ=0.80 min; MS (ESIpos): m/z=369.2 [M+H]⁺.

### Intermediate 291

### 8-ethyl-N-[(4-methoxyphenyl)methyl]-2-(morpholin-4-yl)-N-[(1-{[2-(trimethylsilyl)ethoxy]methyl}-1H-benzimidazol-2-yl)methyl]pyrazolo[1,5-a][1,3,5]triazin-4-amine

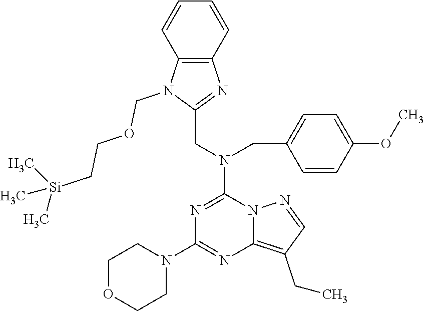

The reaction was performed in two separate batches. To a solution of 8-ethyl-N-[(4-methoxyphenyl)methyl]-2-(morpholin-4-yl)pyrazolo[1,5-a][1,3,5]triazin-4-amine (Intermediate 290, 100/100 mg, 0.27/0.27 mmol) in N,N-dimethylformamide (1.0/2.0 mL) was added sodium hydride (19.0/19.0 mg, 60% purity in mineral oil) at room temperature. After stirring at 60° C. for 15 minutes, 2-(chloromethyl)-1-{[2-(trimethylsilyl)ethoxy]methyl}-1H-benzimidazole (Intermediate 130, 105/105 mg, 0.35/0.35 mmol) was added in one portion. The reaction mixture was stirred at room temperature for 16 hours. The mixtures were combined, diluted with water and extracted with ethyl acetate. The organic phase was washed with brine, dried over anhydrous sodium sulfate and filtered. The filtrate was concentrated to give 8-ethyl-N-(4-methoxybenzyl)-2-(morpholin-4-yl)-N-[(1-{[2-(trimethylsilyl)ethoxy]methyl}-1H-benzimidazol-2-yl)methyl]pyrazolo[1,5-a][1,3,5]triazin-4-amine (500 mg, 70% purity) as yellow oil.

LC-MS (Method D): Rₜ=0.95 min; MS (ESIpos): m/z=629.3 [M+H]⁺.

### Intermediate 292

### N-[(4,5-difluoro-1-{[2-(trimethylsilyl)ethoxy]methyl}-1H-benzimidazol-2-yl)methyl]-8-ethyl-N-(4-methoxybenzyl)-2-(morpholin-4-yl)pyrazolo[1,5-a][1,3,5]triazin-4-amine

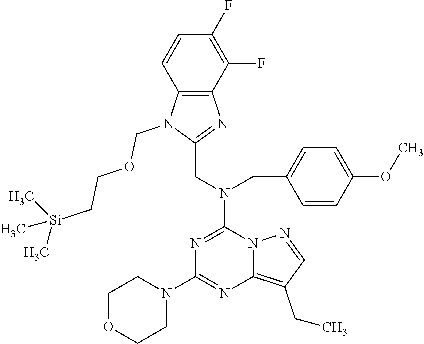

To a solution of 8-ethyl-N-[(4-methoxyphenyl)methyl]-2-(morpholin-4-yl)pyrazolo[1,5-a][1,3,5]triazin-4-amine (Intermediate 290, 200 mg, 0.54 mmol) in N,N-dimethylformamide (2.0 mL) was added sodium hydride (38.0 mg, 60% purity in mineral oil) at room temperature. After stirring at 60° C. for 15 minutes, 2-(chloromethyl)-4,5-difluoro-1-{[2-(trimethylsilyl)ethoxy]methyl}-1H-benzimidazole (Intermediate 158, 235 mg, 0.71 mmol) was added. The reaction mixture was stirred at room temperature for 16 hours. The reaction mixture was diluted with water and extracted with ethyl acetate. The organic phase was washed with brine, dried over anhydrous sodium sulfate and filtered. The filtrate was concentrated to give N-[(4,5-difluoro-1-{[2-(trimethylsilyl)ethoxy]methyl}-1H-benzimidazol-2-yl)methyl]-8-ethyl-N-(4-methoxybenzyl)-2-(morpholin-4-yl)pyrazolo[1,5-a][1,3,5]triazin-4-amine (400 mg, 59% purity, 65% yield) as yellow oil.

LC-MS (Method D): Rₜ=1.04 min; MS (ESIpos): m/z=665.3 [M+H]⁺.

### Intermediate 293

### 8-ethyl-N-(4-methoxybenzyl)-N-[(7-methoxy-1-{[2-(trimethylsilyl)ethoxy]methyl}-1H-benzimidazol-2-yl)methyl]-2-(morpholin-4-yl)pyrazolo[1,5-a][1,3,5]triazin-4-amine

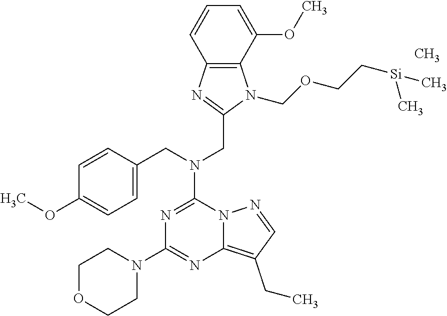

To a solution of 8-ethyl-N-[(4-methoxyphenyl)methyl]-2-(morpholin-4-yl)pyrazolo[1,5-a][1,3,5]triazin-4-amine (Intermediate 290, 100 mg, 0.27 mmol) in N,N-dimethylformamide (2.0 mL) was added sodium hydride (27.1 mg, 60% purity in mineral oil) at room temperature. After stirring at 60° C. for 15 minutes, 2-(chloromethyl)-7-methoxy-1-{[2-(trimethylsilyl)ethoxy]methyl}-1H-benzimidazole (Intermediate 189, 177 mg, 0.54 mmol) was added. The reaction mixture was stirred at room temperature for 16 hours. The reaction mixture was diluted with water and extracted with ethyl acetate. The organic phase was washed with brine, dried over anhydrous sodium sulfate, filtered and concentrated to give a residue. The residue was purified by preperative TLC (petroleum ether: ethyl acetate=1:1) to give 8-ethyl-N-(4-methoxybenzyl)-N-[(7-methoxy-1-{[2-(trimethylsilyl)ethoxy]methyl}-1H-benzimidazol-2-yl)methyl]-2-(morpholin-4-yl)pyrazolo[1,5-a][1,3,5]triazin-4-amine (86.0 mg, 48% yield) as yellow oil.

LC-MS (Method D): Rₜ=0.94 min; MS (ESIpos): m/z=659.3 [M+H]⁺.

### Intermediate 294

### benzyl 4-(4-{bis[(4-methoxyphenyl)methyl]amino}-8-ethylpyrazolo[1,5-a][1,3,5]triazin-2-yl)piperazine-1-carboxylate

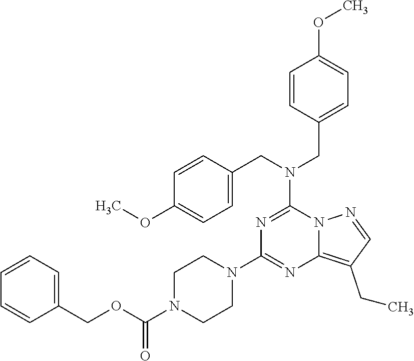 [SMILES: CCc1cnn2c(N(Cc3ccc(OC)cc3)Cc3ccc(OC)cc3)nc(N3CCN(C(=O)OCc4ccccc4)CC3)nc12]

To a solution of benzyl 4-(4-{bis[(4-methoxyphenyl)methyl]amino}-8-ethenylpyrazolo[1,5-a][1,3,5]triazin-2-yl)piperazine-1-carboxylate (Intermediate 275, 1.28 g, 90% purity, 1.86 mmol) in tetrahydrofuran (20 mL) was added palladium (90.0 mg, 10% on activated carbon) at room temperature. The reaction mixture was stirred at room temperature for 16 hours under hydrogen atmosphere (15 psi). The reaction mixture was filtered and the filtrate was concentrated under reduced pressure to give benzyl 4-{4-[bis(4-methoxybenzyl)amino]-8-ethylpyrazolo[1,5-a][1,3,5]triazin-2-yl}piperazine-1-carboxylate (1.50 g, 45% purity, 58% yield) as brown oil.

LC-MS (Method C): Rₜ=1.122 min; MS (ESIpos): m/z=622.4 [M+H]⁺.

### Intermediate 295

### 8-ethyl-N-[(4-methoxyphenyl)methyl]-2-(piperazin-1-yl)pyrazolo[1,5-a][1,3,5]triazin-4-amine

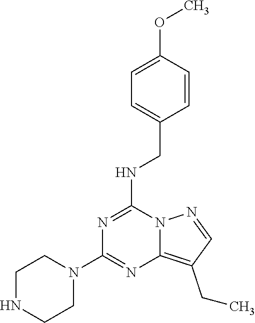 [SMILES: CCc1cnn2c(NCc3ccc(OC)cc3)nc(N3CCNCC3)nc12]

Benzyl 4-(4-{bis[(4-methoxyphenyl)methyl]amino}-8-ethylpyrazolo[1,5-a][1,3,5]triazin-2-yl)piperazine-1-carboxylate (Intermediate 294, 1.50 g, 45% purity, 1.09 mmol) was added to trifluoroacetic acid (15 mL) at room temperature. The solution was stirred at 80° C. for 3 hours. The solution was concentrated to give 8-ethyl-N-(4-methoxybenzyl)-2-(piperazin-1-yl)pyrazolo[1,5-a][1,3,5]triazin-4-amine (1.20 g, 30% purity, 90% yield) as black oil.

LC-MS (Method C): Rₜ=0.784 min; MS (ESIpos): m/z=368.2 [M+H]⁺.

### Intermediate 296

### benzyl 4-(8-ethyl-4-{[(4-methoxyphenyl)methyl]amino}pyrazolo[1,5-a][1,3,5]triazin-2-yl)piperazine-1-carboxylate

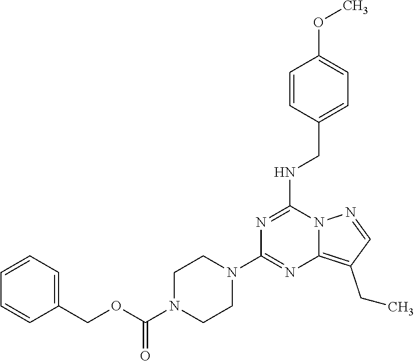 [SMILES: CCc1cnn2c(NCc3ccc(OC)cc3)nc(N3CCN(C(=O)OCc4ccccc4)CC3)nc12]

To a solution of 8-ethyl-N-[(4-methoxyphenyl)methyl]-2-(piperazin-1-yl)pyrazolo[1,5-a][1,3,5]triazin-4-amine (Intermediate 295, 1.20 g, 30% purity, 0.980 mmol) and sodium bicarbonate (247 mg, 2.94 mmol) in a mixed solvent of tetrahydrofuran (15 mL) and water (15 mL) was added benzyl carbonochloridate (251 mg, 1.47 mmol) at 0° C. After stirring at 25° C. for 3 hours, the solution was diluted with water and extracted with ethyl acetate. The combined organic layers were washed with water and brine, dried over anhydrous sodium sulfate and concentrated to give a crude product. The crude product was purified by silica gel column chromatography (200-300 mesh, petroleum ether: ethyl acetate=10:1, then 5:1, then 3:1) to give benzyl 4-{8-ethyl-4-[(4-methoxybenzyl)amino]pyrazolo[1,5-a][1,3,5]triazin-2-yl}piperazine-1-carboxylate (450 mg, 78% purity, 71% yield) as yellow oil.

LC-MS (Method C): Rₜ=0.916 min; MS (ESIpos): m/z=502.3 [M+H]⁺.

### Intermediate 297

### benzyl 4-(8-ethyl-4-{[(4-methoxyphenyl)methyl][(1-{[2-(trimethylsilyl)ethoxy]methyl}-1H-benzimidazol-2-yl)methyl]amino}pyrazolo[1,5-a][1,3,5]triazin-2-yl)piperazine-1-carboxylate

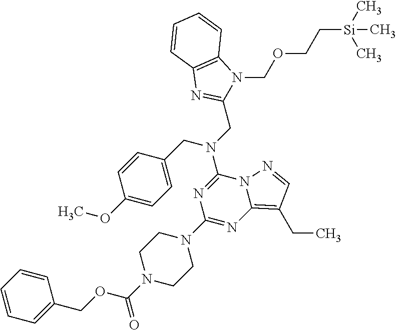 [SMILES: CCc1cnn2c(N(Cc3ccc(OC)cc3)Cc3nc4ccccc4n3COCC[Si](C)(C)C)nc(N3CCN(C(=O)OCc4ccccc4)CC3)nc12]

The reaction was performed in two separate batches. To a solution of benzyl 4-(8-ethyl-4-{[(4-methoxyphenyl)methyl]amino}pyrazolo[1,5-a][1,3,5]triazin-2-yl)piperazine-1-carboxylate (Intermediate 296, 300/150 mg, 78% purity, 0.466/0.233 mmol) in N,N-dimethylformamide (6.0/3.0 mL) was added sodium hydride (56.0/28.0 mg, 60% purity, 1.40/0.700 mmol) at 0° C. After stirring at 60° C. for 15 minutes, 2-(chloromethyl)-1-{[2-(trimethylsilyl)ethoxy]methyl}-1H-benzimidazole (Intermediate 130, 330/165 mg, 84% purity, 0.933/0.467 mmol) was added to the above mixture. The mixture was stirred at 25° C. for 16 hours. The reaction mixtures were combined and poured into water. The solution was extracted with ethyl acetate. The organic phase was washed with brine, dried over anhydrous sodium sulfate and filtered, concentrated to give a crude product. The crude product was purified by silica gel column chromatography (200-300 mesh, petroleum ether: ethyl acetate=10:1, then 3:1, then 1:1) to give benzyl 4-(8-ethyl-4-{(4-methoxybenzyl)[(1-{[2-(trimethylsilyl)ethoxy]methyl}-1H-benzimidazol-2-yl)methyl]amino}pyrazolo[1,5-a][1,3,5]triazin-2-yl)piperazine-1-carboxylate (80.0 mg, 71% purity, 32% yield) as yellow oil.

LC-MS (Method C): Rₜ=1.115 min; MS (ESIpos): m/z=762.5 [M+H]⁺.

### Intermediate 298

### 8-ethyl-N-[(4-methoxyphenyl)methyl]-2-(4-methylpiperazin-1-yl)-N-[(1-{[2-(trimethylsilyl)ethoxy]methyl}-1H-benzimidazol-2-yl)methyl]pyrazolo[1,5-a][1,3,5]triazin-4-amine

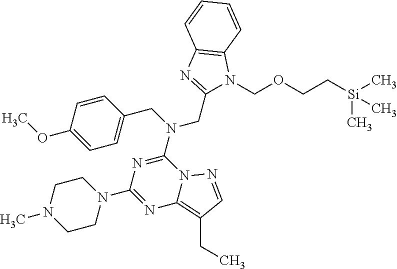

To a solution of benzyl 4-(8-ethyl-4-{[(4-methoxyphenyl)methyl][(1-{[2-(trimethylsilyl)ethoxy] methyl}-1H-benzimidazol-2-yl)methyl]amino}pyrazolo[1,5-a][1,3,5]triazin-2-yl)piperazine-1-carboxylate (Intermediate 297, 80.0 mg, 70% purity, 0.074 mmol) in methanol (2.0 mL) were added formaldehyde (0.01 mL, 37% purity, 0.110 mmol) and palladium (50.0 mg, 10% on activated carbon) at 25° C. The mixture was stirred at 25° C. for 16 hours under hydrogen atmosphere. The reaction mixture was filtered and the filtrate was concentrated under reduced pressure to give 8-ethyl-N-(4-methoxybenzyl)-2-(4-methylpiperazin-1-yl)-N-[(1-{[2-(trimethylsilyl)ethoxy]methyl}-1H-benzimidazol-2-yl)methyl]pyrazolo[1,5-a][1,3,5]triazin-4-amine (40.0 mg, 74% purity, 63% yield) as yellow oil.

LC-MS (Method C): Rₜ=0.848 min; MS (ESIpos): m/z=642.4 [M+H]⁺.

### Intermediate 299

### benzyl 4-(4-{[(4,5-difluoro-1-{[2-(trimethylsilyl)ethoxy]methyl}-1H-benzimidazol-2-yl)methyl][(4-methoxyphenyl)methyl]amino}-8-ethylpyrazolo[1,5-a][1,3,5]triazin-2-yl)piperazine-1-carboxylate

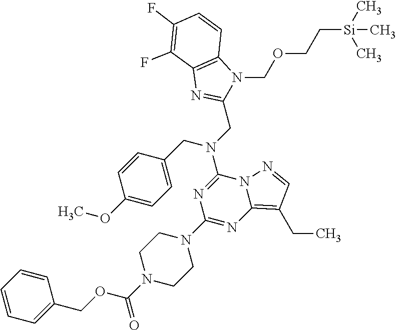 [SMILES: CCc1cnn2c(N(Cc3ccc(OC)cc3)Cc3nc4c(F)c(F)ccc4n3COCC[Si](C)(C)C)nc(N3CCN(C(=O)OCc4ccccc4)CC3)nc12]

The reaction was performed in two separate batches. To a solution of benzyl 4-(8-ethyl-4-{[(4-methoxyphenyl)methyl]amino}pyrazolo[1,5-a][1,3,5]triazin-2-yl)piperazine-1-carboxylate (Intermediate 296, 300/200 mg, 80% purity, 0.478/0.319 mmol) in N,N-dimethylformamide (8.0/3.0 mL) was added sodium hydride (38.3/25.5 mg, 0.957/0.638 mmol, 60% purity in mineral oil) at 25° C. After stirring at 60° C. for 20 minutes, a solution of 2-(chloromethyl)-4,5-difluoro-1-{[2-(trimethylsilyl)ethoxy]methyl}-1H-benzimidazole (Intermediate 158, 325/217 mg, 98% purity, 0.957/0.638 mmol) in N,N-dimethylformamide (2.0 mL) was added at 60° C. After stirring at 60° C. for 16 hours, the reaction mixtures were combined, diluted with water and extracted with ethyl acetate. The combined organic layers were washed with water and brine, dried over anhydrous sodium sulfate and concentrated to give a crude product. The crude was purified by preparative TLC (petroleum ether: ethyl acetate=1:1) to give benzyl 4-(4-{[(4,5-difluoro-1-{[2-(trimethylsilyl)ethoxy]methyl}-1H-benzimidazol-2-yl)methyl] (4-methoxybenzyl)amino}-8-ethylpyrazolo[1,5-a][1,3,5]triazin-2-yl)piperazine-1-carboxylate (250 mg, 68% purity, 67% yield) as yellow oil.

LC-MS (Method C): Rₜ=1.107 min; MS (ESIpos): m/z=798.5 [M+H]⁺.

### Intermediate 300

### N-[(4,5-difluoro-1-{[2-(trimethylsilyl)ethoxy]methyl}-1H-benzimidazol-2-yl)methyl]-8-ethyl-N-(4-methoxybenzyl)-2-(4-methylpiperazin-1-yl)pyrazolo[1,5-a][1,3,5]triazin-4-amine

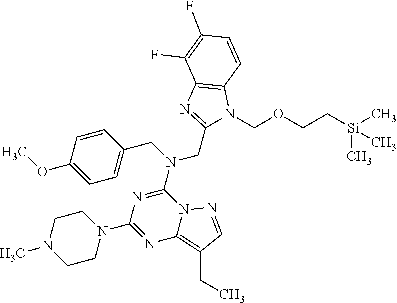

To a solution of benzyl 4-(4-{[(4,5-difluoro-1-{[2-(trimethylsilyl)ethoxy]methyl}-1H-benzimidazol-2-yl)methyl][(4-methoxyphenyl)methyl]amino}-8-ethylpyrazolo[1,5-a][1,3,5]triazin-2-yl)piperazine-1-carboxylate (Intermediate 299, 250 mg, 68% purity, 0.213 mmol), formaldehyde (24 μL, 37% purity, 0.320 mmol) in methanol (5.0 mL) was added palladium (100 mg, 0.094 mmol, 10% purity on carbon) at 25° C. After stirring at 25° C. for 16 hours under hydrogen atmosphere (15 psi). The reaction mixture was filtered. The filtrate was concentrated under reduced pressure to give N-[(4,5-difluoro-1-{[2-(trimethylsilyl)ethoxy]methyl}-1H-benzimidazol-2-yl)methyl]-8-ethyl-N-(4-methoxybenzyl)-2-(4-methylpiperazin-1-yl)pyrazolo[1,5-a][1,3,5] triazin-4-amine (220 mg, 51% purity, 78% yield) as yellow oil.

LC-MS (Method C): Rₜ=0.906 min; MS (ESIpos): m/z=678.5 [M+H]⁺.

### Intermediate 301

### benzyl 4-(4-{bis[(4-methoxyphenyl)methyl]amino}-8-formylpyrazolo[1,5-a][1,3,5]triazin-2-yl)piperazine-1-carboxylate

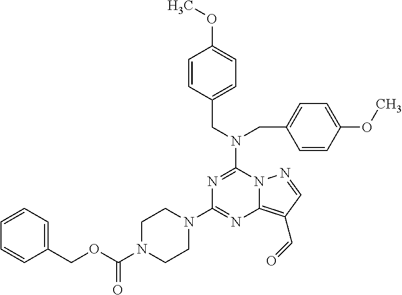 [SMILES: COc1ccc(CN(Cc2ccc(OC)cc2)c2nc(N3CCN(C(=O)OCc4ccccc4)CC3)nc3c(C=O)cnn23)cc1]

To a solution benzyl 4-(4-{bis[(4-methoxyphenyl)methyl]amino}-8-iodopyrazolo[1,5-a][1,3,5] triazin-2-yl)piperazine-1-carboxylate (Intermediate 235, 3.00 g, 4.17 mmol) in tetrahydrofuran (60 mL) was added isopropylmagnesium chloride (4.2 mL, 8.3 mmol, 2.0 M in tetrahydrofuran) at 0° C. After stirring at 25° C. for 1 hour, N,N-dimethylformamide (3.0 mL) was added dropwise to the above mixture, and stirred at 25° C. for another 1 hour. The solution was concentrated and diluted with water, extracted with ethyl acetate. The organic phase was washed with brine, dried over anhydrous sodium sulfate and filtered. The filtrate was concentrated and purified by silica gel column chromatography (200-300 mesh, petroleum ether: ethyl acetate=10:1, then 3:1, then 1:1) to give benzyl 4-{4-[bis(4-methoxybenzyl)amino]-8-formylpyrazolo[1,5-a][1,3,5]triazin-2-yl}piperazine-1-carboxylate (1.60 g, 96% purity, 59% yield) as yellow oil.

LC-MS (Method C): Rₜ=1.005 min; MS (ESIpos): m/z=622.3 [M+H]⁺.

### Intermediate 302

### benzyl 4-{4-[bis(4-methoxybenzyl)amino]-8-(2,2,2-trifluoro-1-hydroxyethyl)pyrazolo[1,5-a][1,3,5]triazin-2-yl}piperazine-1-carboxylate

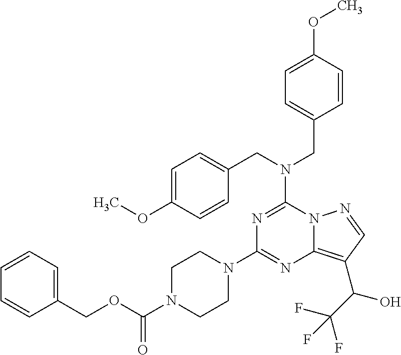 [SMILES: COc1ccc(CN(Cc2ccc(OC)cc2)c2nc(N3CCN(C(=O)OCc4ccccc4)CC3)nc3c(C(O)C(F)(F)F)cnn23)cc1]

The reaction was performed in two separate batches. To a solution of benzyl 4-(4-{bis[(4-methoxyphenyl)methyl]amino}-8-formylpyrazolo[1,5-a][1,3,5]triazin-2-yl)piperazine-1-carboxylate (Intermediate 301, 0.20/1.40 g, 96% purity, 0.309/2.16 mmol) and cesium fluoride (140/985 mg, 0.926/6.49 mmol) in tetrahydrofuran (8.0/50 mL) was added dropwise trimethyl(trifluoromethyl)silane (131/922 mg, 0.926/6.49 mmol) at 0° C. After stirring at 25° C. for 16 hours, the reaction mixtures were combined, diluted with water and extracted with ethyl acetate. The combined organic layers were washed with water and brine, dried over anhydrous sodium sulfate and concentrated to give benzyl 4-{4-[bis(4-methoxybenzyl)amino]-8-(2,2,2-trifluoro-1-hydroxyethyl)pyrazolo[1,5-a][1,3,5]triazin-2-yl}piperazine-1-carboxylate (1.30 g, 92% purity, 80% yield) as yellow oil.

LC-MS (Method C): Rₜ=1.047 min; MS (ESIpos): m/z=692.3 [M+H]⁺.

### Intermediate 303

### N-[(4-methoxyphenyl)methyl]-2-(piperazin-1-yl)-8-(2,2,2-trifluoroethyl)pyrazolo[1,5-a][1,3,5] triazin-4-amine

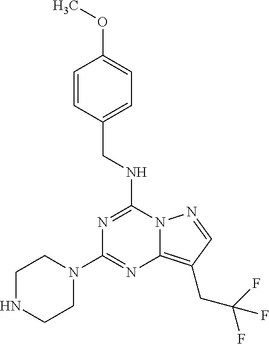 [SMILES: COc1ccc(CNc2nc(N3CCNCC3)nc3c(CC(F)(F)F)cnn23)cc1]

To a solution of benzyl 4-{4-[bis(4-methoxybenzyl)amino]-8-(2,2,2-trifluoro-1-hydroxyethyl)pyrazolo[1,5-a][1,3,5]triazin-2-yl}piperazine-1-carboxylate (Intermediate 302, 1.30 g, 92% purity, 1.73 mmol) in trifluoroacetic acid (13 mL, 170 mmol) was added triethylsilane (6.5 mL) at room temperature. The reaction mixture was stirred at 80° C. for 3 hours. The solution was diluted with water, adjusted to pH=7 by sodium bicarbonate and extracted with ethyl acetate. The organic phase was washed with brine, dried over anhydrous sodium sulfate and filtered. The filtrate was concentrated and purified by silica gel column chromatography (200-300 mesh, petroleum ether: ethyl acetate=10:1, then 3:1, then 1:1) to give N-(4-methoxybenzyl)-2-(piperazin-1-yl)-8-(2,2,2-trifluoroethyl)pyrazolo[1,5-a][1,3,5]triazin-4-amine (850 mg, 98% yield) as brown oil.

LC-MS (Method C): Rₜ=0.823 min; MS (ESIpos): m/z=422.4 [M+H]⁺.

### Intermediate 304

### benzyl 4-[4-{[(4-methoxyphenyl)methyl]amino}-8-(2,2,2-trifluoroethyl)pyrazolo[1,5-a][1,3,5] triazin-2-yl]piperazine-1-carboxylate

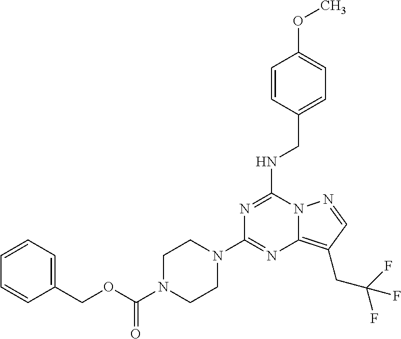 [SMILES: COc1ccc(CNc2nc(N3CCN(C(=O)OCc4ccccc4)CC3)nc3c(CC(F)(F)F)cnn23)cc1]

The reaction was performed in two separate batches. To a solution of N-[(4-methoxyphenyl)methyl]-2-(piperazin-1-yl)-8-(2,2,2-trifluoroethyl)pyrazolo [1,5-a][1,3,5]triazin-4-amine (Intermediate 303, 100/750 mg, 84% purity, 0.201/1.49 mmol) and sodium bicarbonate (50.7/377 mg, 0.603/4.48 mmol) in a mixed solvent of tetrahydrofuran (1.5/10 mL) and water (1.5/10 mL) was added dropwise benzyl carbonochloridate (51.4/383 mg, 0.301/2.24 mmol) at 0° C. The mixture was stirred at 25° C. for 3 hours. The solution a were combined, diluted with water and extracted with ethyl acetate. The combined organic layers were washed with water and brine, dried over anhydrous sodium sulfate and concentrated to give a crude product. The crude product was purified by silica gel column chromatography (100-200 mesh, petroleum ether: ethyl acetate=10:1, then 5:1, then 3:1) to give benzyl 4-{4-[(4-methoxybenzyl)amino]-8-(2,2,2-trifluoroethyl)pyrazolo[1,5-a][1,3,5]triazin-2-yl}piperazine-1-carboxylate (560 mg, 53% yield) as yellow oil.

LC-MS (Method C): Rₜ=1.060 min; MS (ESIpos): m/z=556.5 [M+H]⁺.

### Intermediate 305

### benzyl 4-[4-{[(4-methoxyphenyl)methyl][(1-{[2-(trimethylsilyl)ethoxy]methyl}-1H-benzimidazol-2-yl)methyl]amino}-8-(2,2,2-trifluoroethyl)pyrazolo[1,5-a][1,3,5]triazin-2-yl]piperazine-1-carboxylate

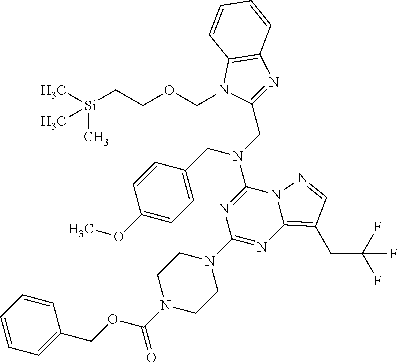 [SMILES: COc1ccc(CN(Cc2nc3ccccc3n2COCC[Si](C)(C)C)c2nc(N3CCN(C(=O)OCc4ccccc4)CC3)nc3c(CC(F)(F)F)cnn23)cc1]

To a solution of benzyl 4-[4-{[(4-methoxyphenyl)methyl]amino}-8-(2,2,2-trifluoroethyl) pyrazolo[1,5-a][1,3,5]triazin-2-yl]piperazine-1-carboxylate (Intermediate 304, 186 mg, 79% purity, 0.26 mmol) in N,N-dimethylformamide (4.0 mL) was added sodium hydride (21.2 mg, 0.53 mmol, 60% purity in mineral oil) at room temperature. After stirring at 60° C. for 20 minutes, 2-(chloromethyl)-1-{[2-(trimethylsilyl)ethoxy]methyl}-1H-benzimidazole (Intermediate 130, 141 mg, 84% purity, 0.40 mmol) was added. After stirring at room temperature for 16 hours, the reaction mixture was poured into water, extracted with ethyl acetate. The organic phase was washed with brine, dried over anhydrous sodium sulfate and filtered. The filtrate was concentrated to give a crude product. The crude product was purified by TLC (dichloromethane:methanol=20:1) to give benzyl 4-[4-{(4-methoxybenzyl)[(1-{[2-(trimethylsilyl)ethoxy]methyl}-1H-benzimidazol-2-yl)methyl]amino}-8-(2,2,2-trifluoroethyl)pyrazolo[1,5-a][1,3,5]triazin-2-yl]piperazine-1-carboxylate (200 mg, 52% yield) as yellow oil.

LC-MS (Method C): Rₜ=1.120 min; MS (ESIpos): m/z=816.7 [M+H]⁺.

### Intermediate 306

### N-[(4-methoxyphenyl)methyl]-2-(4-methylpiperazin-1-yl)-8-(2,2,2-trifluoroethyl)-N-[(1-{[2-(trimethylsilyl)ethoxy]methyl}-1H-benzimidazol-2-yl)methyl]pyrazolo[1,5-a][1,3,5]triazin-4-amine

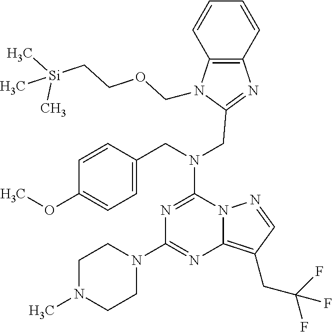 [SMILES: COc1ccc(CN(Cc2nc3ccccc3n2COCC[Si](C)(C)C)c2nc(N3CCN(C)CC3)nc3c(CC(F)(F)F)cnn23)cc1]

To a solution of benzyl 4-[4-{[(4-methoxyphenyl)methyl][(1-{[2-(trimethylsilyl)ethoxy]methyl}-1H-benzimidazol-2-yl)methyl]amino}-8-(2,2,2-trifluoroethyl)pyrazolo[1,5-a][1,3,5]triazin-2-yl]piperazine-1-carboxylate (Intermediate 305, 200 mg, 54% purity, 0.13 mmol) and formaldehyde (42.9 mg, 0.53 mmol, 37% purity in water) in methanol (5.0 mL) was added palladium (50.0 mg, 10% on activated carbon) at 25° C. After stirring at 25° C. for 16 hours under hydrogen atmosphere (15 psi). The reaction mixture was filtered. The filtrate was concentrated and purified by preparative TLC (dichloromethane: methyl alcohol=20:1) to give N-(4-methoxybenzyl)-2-(4-methylpiperazin-1-yl)-8-(2,2,2-trifluoroethyl)-N-[(1-{[2-(trimethylsilyl)ethoxy]methyl}-1H-benzimidazol-2-yl)methyl]pyrazolo[1,5-a][1,3,5]triazin-4-amine (60.0 mg, 47% yield) as yellow oil.

LC-MS (Method C): Rₜ=0.934 min; MS (ESIpos): m/z=696.6 [M+H]⁺.

### Intermediate 307

### benzyl 4-[4-{[(4,5-difluoro-1-{[2-(trimethylsilyl)ethoxy]methyl}-1H-benzimidazol-2-yl)methyl][(4-methoxyphenyl)methyl]amino}-8-(2,2,2-trifluoroethyl)pyrazolo[1,5-a][1,3,5]triazin-2-yl]piperazine-1-carboxylate

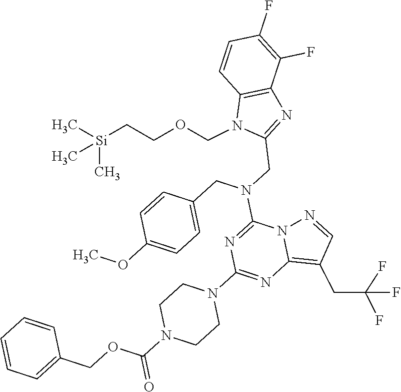 [SMILES: COc1ccc(CN(Cc2nc3c(F)c(F)ccc3n2COCC[Si](C)(C)C)c2nc(N3CCN(C(=O)OCc4ccccc4)CC3)nc3c(CC(F)(F)F)cnn23)cc1]

To a solution of benzyl 4-[4-{[(4-methoxyphenyl)methyl]amino}-8-(2,2,2-trifluoroethyl)pyrazolo [1,5-a][1,3,5]triazin-2-yl]piperazine-1-carboxylate (Intermediate 304, 186 mg, 79% purity, 0.26 mmol) in N,N-dimethylformamide (4.0 mL) was added sodium hydride (21.2 mg, 0.53 mmol, 60% purity in mineral oil) at room temperature. After stirring at 60° C. for 20 minutes, 2-(chloromethyl)-4,5-difluoro-1-{[2-(trimethylsilyl)ethoxy]methyl}-1H-benzimidazole (Intermediate 158, 134 mg, 99% purity, 0.40 mmol) was added. After stirring at room temperature for 16 hours, the mixture was poured into water, extracted with ethyl acetate. The organic phase was washed with water and brine, dried over anhydrous sodium sulfate and filtered. The filtrate was concentrated and purified by TLC (dichloromethane: methyl alcohol=20:1) to give benzyl 4-[4-{[(4,5-difluoro-1-{[2-(trimethylsilyl)ethoxy]methyl}-1H-benzimidazol-2-yl)methyl](4-methoxybenzyl)amino}-8-(2,2,2-trifluoroethyl)pyrazolo[1,5-a][1,3,5]triazin-2-yl]piperazine-1-carboxylate (200 mg, 42% purity, 37% yield) as yellow oil.

LC-MS (Method C): Rₜ=1.254 min; MS (ESIpos): m/z=852.6 [M+H]⁺.

### Intermediate 308

### N-[(4,5-difluoro-1-{[2-(trimethylsilyl)ethoxy]methyl}-1H-benzimidazol-2-yl)methyl]-N-[(4-methoxyphenyl)methyl]-2-(4-methylpiperazin-1-yl)-8-(2,2,2-trifluoroethyl)pyrazolo[1,5-a][1,3,5] triazin-4-amine

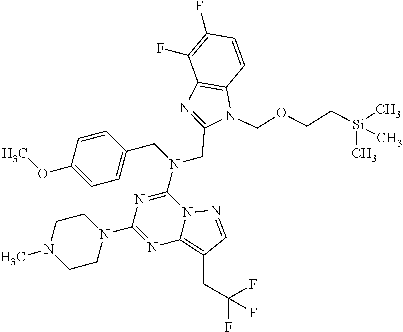

To a solution of benzyl 4-[4-{[(4,5-difluoro-1-{[2-(trimethylsilyl)ethoxy]methyl}-1H-benzimidazol-2-yl)methyl][(4-methoxyphenyl)methyl]amino}-8-(2,2,2-trifluoroethyl)pyrazolo[1,5-a][1,3,5] triazin-2-yl]piperazine-1-carboxylate (Intermediate 307, 200 mg, 41% purity, 0.09 mmol) and formaldehyde (31.2 mg, 0.39 mmol, 37% purity in water) in methanol (5.0 mL) was added palladium (50.0 mg, 10% on activated carbon) at 25° C. The mixture was stirred at 25° C. for 16 hours under hydrogen atmosphere (15 psi). The mixture was filtered, and the filtrate was concentrated, purified by preparative TLC (dichloromethane:methanol=20:1) to give N-[(4,5-difluoro-1-{[2-(trimethylsilyl)ethoxy]methyl}-1H-benzimidazol-2-yl)methyl]-N-(4-methoxybenzyl)-2-(4-methylpiperazin-1-yl)-8-(2,2,2-trifluoroethyl)pyrazolo[1,5-a][1,3,5]triazin-4-amine (60.0 mg, 94% purity, 80% yield) as yellow oil.

LC-MS (Method C): Rₜ=1.010 min; MS (ESIpos): m/z=732.6 [M+H]⁺.

### Intermediate 309

### N-[(4-methoxyphenyl)methyl]-2-(morpholin-4-yl)-8-(pyridazin-4-yl)-N-[(1-{[2-(trimethylsilyl) ethoxy]methyl}-1H-benzimidazol-2-yl)methyl]pyrazolo[1,5-a][1,3,5]triazin-4-amine

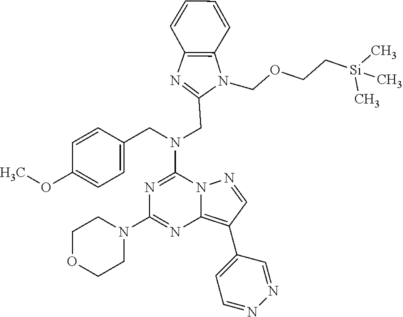

The reaction was performed in two separate batches. To a solution of N-[(4-methoxyphenyl)methyl]-2-(morpholin-4-yl)-8-(pyridazin-4-yl)pyrazolo[1,5-a][1,3,5]triazin-4-amine (Intermediate 244, 20/200 mg, 94% purity, 0.040/0.401 mmol) in N,N-dimethylformamide (0.5/5.0 mL) were added potassium carbonate (16.6/166 mg, 0.12/1.20 mmol) and 2-(chloromethyl)-1-{[2-(trimethylsilyl)ethoxy]methyl}-1H-benzimidazole (Intermediate 130, 14.9/149 mg, 96% purity, 0.048/0.482 mmol) at room temperature. After stirring at 60° C. for 16 hours, the reaction mixtures were combined, diluted with water and extracted with ethyl acetate. The combined organic layers were washed with water and brine, dried over anhydrous sodium sulfate and concentrated to give a residue. The residue was purified by preparative TLC (petroleum ether: ethyl acetate=1:1) to give N-(4-methoxybenzyl)-2-(morpholin-4-yl)-8-(pyridazin-4-yl)-N-[(1-{[2-(trimethylsilyl)ethoxy]methyl}-1H-benzimidazol-2-yl)methyl]pyrazolo[1,5-a][1,3,5]triazin-4-amine (200 mg, 67% purity, 49% yield) as a brown solid.

LC-MS (Method C): Rₜ=0.89 min; MS (ESIpos): m/z=679.3 [M+H]⁺.

### Intermediate 310

### N-[(4-methoxyphenyl)methyl]-N-[(7-methoxy-1-{[2-(trimethylsilyl)ethoxy]methyl}-1H-benzimidazol-2-yl)methyl]-2-(morpholin-4-yl)-8-(pyridazin-4-yl)pyrazolo[1,5-a][1,3,5]triazin-4-amine

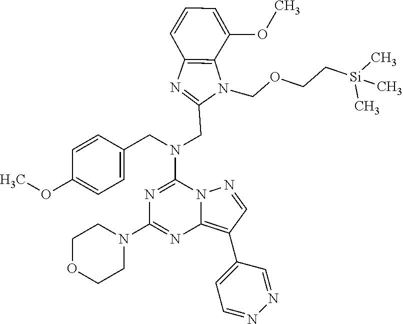

To a solution of N-[(4-methoxyphenyl)methyl]-2-(morpholin-4-yl)-8-(pyridazin-4-yl)pyrazolo[1,5-a][1,3,5]triazin-4-amine (Intermediate 244, 200 mg, 0.478 mmol) in N,N-dimethylformamide (2.0 mL) were added potassium carbonate (132 mg, 0.956 mmol) and 2-(chloromethyl)-7-methoxy-1-{[2-(trimethylsilyl)ethoxy]methyl}-1H-benzimidazole (Intermediate 189, 172 mg, 0.526 mmol) at room temperature. After stirring at 60° C. for 16 hours, the reaction mixture was diluted with water and extracted with ethyl acetate. The organic phase was washed with brine, dried over anhydrous sodium sulfate, and concentrated to give N-(4-methoxybenzyl)-N-[(7-methoxy-1-{[2-(trimethylsilyl)ethoxy]methyl}-1H-benzimidazol-2-yl)methyl]-2-(morpholin-4-yl)-8-(pyridazin-4-yl)pyrazolo[1,5-a][1,3,5]triazin-4-amine (310 mg, 91% yield) as a yellow solid.

LC-MS (Method D): Rₜ=0.861 min; MS (ESIpos): m/z=709.3 [M+H]⁺.

### Intermediate 311

### N-[(5,6-dichloro-1-{[2-(trimethylsilyl)ethoxy]methyl}-1H-benzimidazol-2-yl)methyl]-N-[(4-methoxyphenyl)methyl]-2-(morpholin-4-yl)-8-(pyridazin-4-yl)pyrazolo[1,5-a][1,3,5]triazin-4-amine

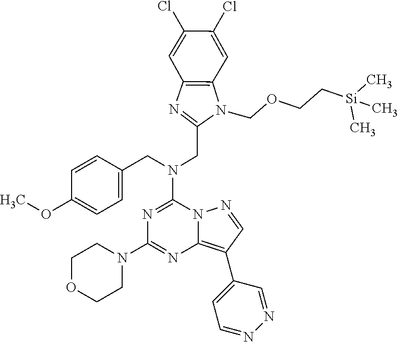

To a solution of N-[(4-methoxyphenyl)methyl]-2-(morpholin-4-yl)-8-(pyridazin-4-yl)pyrazolo[1,5-a][1,3,5]triazin-4-amine (Intermediate 244, 200 mg, 0.478 mmol) in N,N-dimethylformamide (2.0 mL) were added potassium carbonate (132 mg, 0.956 mmol) and 5,6-dichloro-2-(chloromethyl)-1-{[2-(trimethylsilyl)ethoxy]methyl}-1H-benzimidazole (Intermediate 250, 210 mg, 0.574 mmol) at room temperature. After stirring at 50° C. for 16 hours, the mixture was quenched with saturated ammonium chloride aqueous solution and extracted with ethyl acetate. The organic phase was washed with brine, dried over anhydrous sodium sulfate, and concentrated to give a residue. The residue was purified by preparative TLC (petroleum ether: ethyl acetate=1:1) to give N-((5,6-dichloro-1-((2-(trimethylsilyl)ethoxy)methyl)-1H-benzo[d]imidazol-2-yl)methyl)-N-(4-methoxybenzyl)-2-morpholino-8-(pyridazin-4-yl)pyrazolo[1,5-a][1,3,5]triazin-4-amine (128 mg, 36% yield) as brown oil.

LC-MS (Method D): Rₜ=0.924 min; MS (ESIpos): m/z=747.1 [M+H]⁺.

### Intermediate 312

### N-[(4-methoxyphenyl)methyl]-2-(morpholin-4-yl)-8-(1,3-thiazol-5-yl)-N-[(1-{[2-(trimethylsilyl)ethoxy]methyl}-1H-benzimidazol-2-yl)methyl]pyrazolo[1,5-a][1,3,5]triazin-4-amine

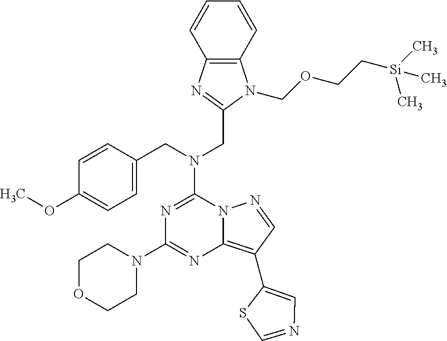

The reaction was performed in two separate batches. To a solution of N-[(4-methoxyphenyl)methyl]-2-(morpholin-4-yl)-8-(1,3-thiazol-5-yl)pyrazolo [1,5-a][1,3,5]triazin-4-amine (Intermediate 264, 20/200 mg, 83% purity, 0.039/0.39 mmol) in N,N-dimethylformamide (0.5/4.0 mL) were added potassium carbonate (16.3/163 mg, 0.118/1.18 mmol) and 2-(chloromethyl)-1-{[2-(trimethylsilyl)ethoxy]methyl}-1H-benzimidazole (Intermediate 130, 14.5/145 mg, 96% purity, 0.047/0.47 mmol) at room temperature. After stirring at 60° C. for 16 hours, the combined reaction mixtures were diluted with water and extracted with ethyl acetate. The combined organic layers were washed with water and brine, dried over anhydrous sodium sulfate and concentrated to give a residue. The residue was purified by preparative TLC (petroleum ether: ethyl acetate=3:1) to give N-(4-methoxybenzyl)-2-(morpholin-4-yl)-8-(1,3-thiazol-5-yl)-N-[(1-{[2-(trimethylsilyl)ethoxy]methyl}-1H-benzimidazol-2-yl)methyl]pyrazolo[1,5-a][1,3,5]triazin-4-amine (230 mg, 91% purity, 78% yield) as brown oil.

LC-MS (Method D): Rₜ=0.91 min; MS (ESIpos): m/z=684.3 [M+H]⁺.

### Intermediate 313

### N-[(4,5-difluoro-1-{[2-(trimethylsilyl)ethoxy]methyl}-1H-benzimidazol-2-yl)methyl]-N-[(4-methoxyphenyl)methyl]-2-(morpholin-4-yl)-8-(1,3-thiazol-5-yl)pyrazolo[1,5-a][1,3,5]triazin-4-amine

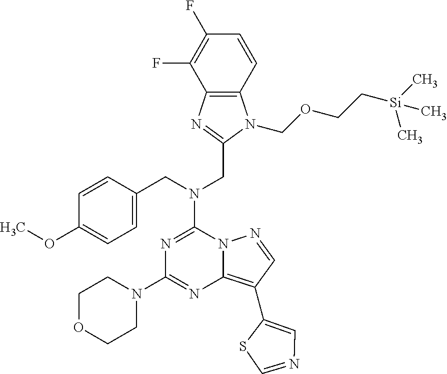

To a solution of N-[(4-methoxyphenyl)methyl]-2-(morpholin-4-yl)-8-(1,3-thiazol-5-yl)pyrazolo [1,5-a][1,3,5]triazin-4-amine (Intermediate 264, 200 mg, 83% purity, 0.39 mmol) in N,N-dimethylformamide (4.0 mL) were added potassium carbonate (108 mg, 0.78 mmol) and 2-(chloromethyl)-4,5-difluoro-1-{[2-(trimethylsilyl)ethoxy]methyl}-1H-benzimidazole (Intermediate 158, 159 mg, 99% purity, 0.47 mmol) at room temperature. After stirring at 60° C. for 16 hours, the reaction mixture was diluted with water and extracted with ethyl acetate. The combined organic layers were washed with water and brine, dried over anhydrous sodium sulfate and concentrated to give N-[(4,5-difluoro-1-{[2-(trimethylsilyl)ethoxy]methyl}-1H-benzimidazol-2-yl)methyl]-N-(4-methoxybenzyl)-2-(morpholin-4-yl)-8-(1,3-thiazol-5-yl)pyrazolo[1,5-a][1,3,5]triazin-4-amine (300 mg, 85% purity, 90% yield) as brown oil.

LC-MS (Method C): Rₜ=1.15 min; MS (ESIpos): m/z=720.2 [M+H]⁺.

### Intermediate 314

### N-[(5,6-dichloro-1-{[2-(trimethylsilyl)ethoxy]methyl}-1H-benzimidazol-2-yl)methyl]-N-[(4-methoxyphenyl)methyl]-2-(morpholin-4-yl)-8-(1,3-thiazol-5-yl)pyrazolo[1,5-a][1,3,5]triazin-4-amine

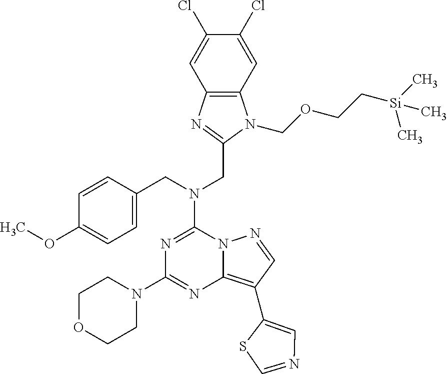

To a solution of N-[(4-methoxyphenyl)methyl]-2-(morpholin-4-yl)-8-(1,3-thiazol-5-yl)pyrazolo[1,5-a][1,3,5]triazin-4-amine (Intermediate 264, 200 mg, 83% purity, 0.39 mmol) in N,N-dimethylformamide (4.0 mL) were added potassium carbonate (108 mg, 0.78 mmol) and 5,6-dichloro-2-(chloromethyl)-1-{[2-(trimethylsilyl)ethoxy]methyl}-1H-benzimidazole (Intermediate 250, 200 mg, 86% purity, 0.47 mmol) at room temperature. After stirring at 50° C. for 16 hours, the reaction mixture was diluted with water and extracted with ethyl actate. The combined organic layer was washed with brine and water, dried over anhydrous sodium sulfate and concentrated to give a residue. The residue was purified by preparative TLC (petroleum ether: ethyl actate=2:1) to give N-[(5,6-dichloro-1-{[2-(trimethylsilyl)ethoxy]methyl}-1H-benzimidazol-2-yl)methyl]-N-(4-methoxybenzyl)-2-(morpholin-4-yl)-8-(1,3-thiazol-5-yl)pyrazolo[1,5-a][1,3,5]triazin-4-amine (190 mg, 84% purity, 54% yield) as yellow oil.

LC-MS (Method C): Rₜ=1.18 min; MS (ESIpos): m/z=752.2 [M+H]⁺.

### Intermediate 315

### 8-(2,2-difluoroethyl)-N-[(4-methoxyphenyl)methyl]-2-(morpholin-4-yl)-N-[(1-{[2-(trimethylsilyl) ethoxy]methyl}-1H-benzimidazol-2-yl)methyl]pyrazolo[1,5-a][1,3,5]triazin-4-amine

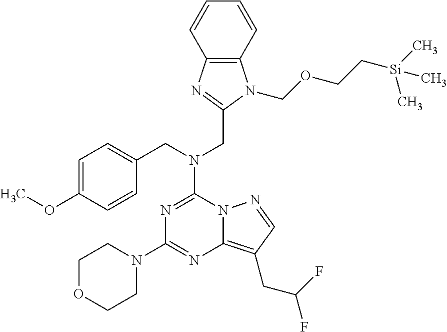

The reaction was performed in two separate batches. To a solution of 8-(2,2-difluoroethyl)-N-[(4-methoxyphenyl)methyl]-2-(morpholin-4-yl)pyrazolo[1,5-a][1,3,5]triazin-4-amine (Intermediate 267, 20/160 mg, 83% purity, 41 μmol/0.33 mmol) in N,N-dimethylformamide (0.5/4.0 mL) were added potassium carbonate (17/136 mg, 123 μmol/0.99 mmol) and 2-(chloromethyl)-1-{[2-(trimethylsilyl)ethoxy]methyl}-1H-benzimidazole (Intermediate 130, 17.5/122 mg, 96% purity, 49 μmol/0.40 mmol) at room temperature. After stirring at 60° C. for 16 hours, the reaction mixtures were combined, diluted with water and extracted with ethyl acetate. The combined organic layers were washed with water and brine, dried over anhydrous sodium sulfate and concentrated to give a residue. The residue was purified by preparative TLC (petroleum ether: ethyl acetate=3:1) to give 8-(2,2-difluoroethyl)-N-(4-methoxybenzyl)-2-(morpholin-4-yl)-N-[(1-{[2-(trimethylsilyl)ethoxy]methyl}-1H-benzimidazol-2-yl)methyl]pyrazolo[1,5-a][1,3,5]triazin-4-amine (180 mg, 99% purity, 82% yield) as yellow oil.

LC-MS (Method C): Rₜ=1.00 min; MS (ESIpos): m/z=665.3 [M+H]⁺.

### Example 1

### N-[(1H-benzimidazol-2-yl)methyl]-2-(morpholin-4-yl)-8-(propan-2-yl)pyrazolo[1,5-a][1,3,5]triazin-4-amine

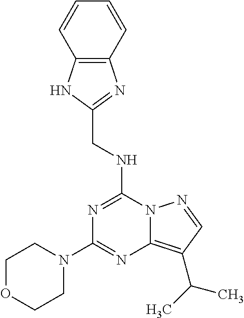 [SMILES: CC(C)c1cnn2c(NCc3nc4ccccc4[nH]3)nc(N3CCOCC3)nc12]

N-[(1H-benzimidazol-2-yl)methyl]-2-(methanesulfonyl)-8-(propan-2-yl)pyrazolo[1,5-a][1,3,5]triazin-4-amine (Intermediate 2, 84.8 mg, 220 μmol) and morpholine (58 μL, 660 μmol; CAS 110-91-8) were dissolved in acetonitrile (2.1 mL), N,N-diisopropylethylamine (110 μL, 660 μmol) was added and the mixture was stirred overnight at 70° C. The mixture was purified by preparative HPLC (HT basic), followed by precipitation from methanol to give 19.5 mg (22% yield) of the title compound.

LC-MS (Method 2): Rₜ=1.14 min; MS (ESIpos): m/z=393 [M+H]⁺

¹H-NMR (400 MHz, DMSO-d6) δ[ppm]: 1.239 (15.37), 1.256 (16.00), 2.518 (2.47), 2.523 (1.68), 2.934 (0.97), 2.952 (1.27), 2.969 (0.92), 3.485 (2.84), 3.495 (2.54), 3.561 (3.06), 3.573 (3.41), 3.583 (1.91), 4.850 (4.03), 7.112 (2.37), 7.119 (2.24), 7.126 (2.33), 7.134 (2.72), 7.144 (0.49), 7.464 (0.84), 7.804 (6.12).

### Example 2

### N-[(1H-benzimidazol-2-yl)methyl]-2-(4-methylpiperazin-1-yl)-8-(propan-2-yl)pyrazolo[1,5-a][1,3,5]triazin-4-amine

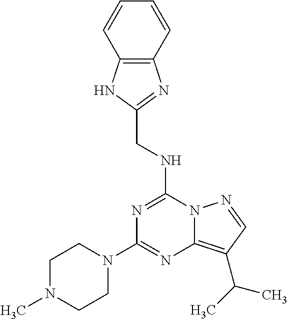 [SMILES: CC(C)c1cnn2c(NCc3nc4ccccc4[nH]3)nc(N3CCN(C)CC3)nc12]

N-[(1H-benzimidazol-2-yl)methyl]-2-(methanesulfonyl)-8-(propan-2-yl)pyrazolo[1,5-a][1,3,5]triazin-4-amine (Intermediate 2, 84.8 mg, 220 μmol) and 1-methylpiperazine (73 μL, 660 μmol; CAS 109-01-3) were dissolved in acetonitrile (2.1 mL), N,N-diisopropylethylamine (110 μL, 660 μmol) was added and the mixture was stirred overnight at 70° C. The mixture was purified by preparative HPLC (HT basic) to give 48.5 mg (53% yield) of the title compound.

LC-MS (Method 2): Rₜ=1.10 min; MS (ESIpos): m/z=406 [M+H]⁺

¹H-NMR (400 MHz, DMSO-d6) δ[ppm]: 1.236 (15.67), 1.253 (16.00), 2.074 (0.63), 2.105 (9.78), 2.169 (2.25), 2.331 (0.50), 2.518 (3.77), 2.523 (2.55), 2.539 (0.58), 2.673 (0.46), 2.927 (0.96), 2.945 (1.28), 2.962 (0.91), 3.583 (2.58), 4.841 (3.36), 7.109 (2.35), 7.117 (2.11), 7.124 (2.25), 7.132 (2.63), 7.142 (0.52), 7.461 (0.73), 7.785 (6.08).

### Example 3

### N⁴-[(1H-benzimidazol-2-yl)methyl]-N²,N²-dimethyl-8-(propan-2-yl)pyrazolo[1,5-a][1,3,5]triazine-2,4-diamine

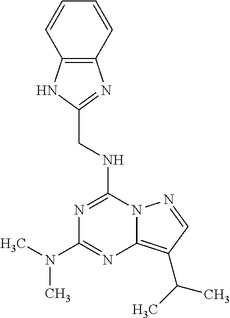 [SMILES: CC(C)c1cnn2c(NCc3nc4ccccc4[nH]3)nc(N(C)C)nc12]

N-[(1H-benzimidazol-2-yl)methyl]-2-(methanesulfonyl)-8-(propan-2-yl)pyrazolo[1,5-a][1,3,5]triazin-4-amine (Intermediate 2, 95.0 mg, 246 μmol) and N-methylmethanamine (370 μL, 2.0 M, 740 μmol) were dissolved in acetonitrile (2.3 mL), N,N-diisopropylethylamine (130 μL, 740 μmol) was added and the mixture was stirred overnight at 70° C. The mixture was purified by preparative HPLC (HT basic) to give 3.50 mg (4% yield) of the title compound.

LC-MS (Method 2): Rₜ=1.26 min; MS (ESIpos): m/z=351 [M+H]⁺

¹H-NMR (400 MHz, DMSO-d6) δ[ppm]: 1.244 (15.65), 1.262 (16.00), 2.327 (1.88), 2.331 (1.37), 2.337 (0.61), 2.518 (11.12), 2.523 (7.55), 2.540 (1.12), 2.669 (1.88), 2.673 (1.34), 2.678 (0.64), 2.934 (0.99), 2.951 (1.31), 2.969 (1.05), 2.994 (14.88), 3.871 (1.43), 4.854 (3.89), 7.105 (2.49), 7.112 (2.33), 7.119 (2.42), 7.127 (2.90), 7.137 (0.51), 7.463 (0.96), 7.760 (5.80).

### Example 4

### N-[(1H-benzimidazol-2-yl)methyl]-8-bromo-2-(morpholin-4-yl)pyrazolo[1,5-a][1,3,5]triazin-4-amine

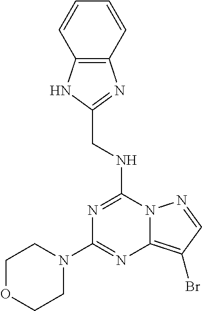 [SMILES: Brc1cnn2c(NCc3nc4ccccc4[nH]3)nc(N3CCOCC3)nc12]

N-[(1H-benzimidazol-2-yl)methyl]-8-bromo-2-(methanesulfonyl)pyrazolo[1,5-a][1,3,5]triazin-4-amine (Intermediate 9, 143 mg, 339 μmol) and morpholine (89 μL, 1.0 mmol; CAS 110-91-8) were dissolved in acetonitrile (3.2 mL), N,N-diisopropylethylamine (180 μL, 1.0 mmol) was added and the mixture was stirred overnight at 70° C. The mixture was purified by preparative HPLC (HT basic) to give 91.0 mg (62% yield) of the title compound.

LC-MS (Method 2): Rₜ=1.01 min; MS (ESIneg): m/z=427 [M−H]⁻

¹H-NMR (400 MHz, DMSO-d6) δ[ppm]: 2.336 (0.65), 2.518 (8.31), 2.522 (5.70), 2.539 (6.28), 2.678 (0.65), 3.305 (0.44), 3.490 (2.94), 3.620 (4.46), 4.867 (3.30), 4.876 (3.27), 7.100 (0.47), 7.114 (1.92), 7.118 (1.49), 7.124 (2.32), 7.131 (2.54), 7.136 (2.58), 7.143 (1.56), 7.147 (2.10), 7.162 (0.51), 7.393 (1.56), 7.410 (1.38), 7.527 (1.45), 7.544 (1.38), 8.037 (16.00), 9.138 (1.12), 12.253 (1.92).

### Example 5

### 1-{[4-{[(1H-benzimidazol-2-yl)methyl]amino}-8-(propan-2-yl)pyrazolo[1,5-a][1,3,5]triazin-2-yl](methyl)amino}-2-methylpropan-2-ol

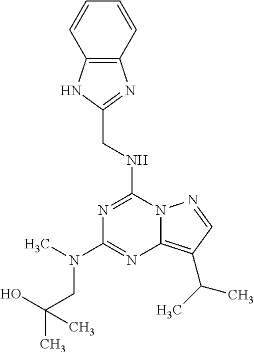 [SMILES: CC(C)c1cnn2c(NCc3nc4ccccc4[nH]3)nc(N(C)CC(C)(C)O)nc12]

N-[(1H-benzimidazol-2-yl)methyl]-2-(methanesulfonyl)-8-(propan-2-yl)pyrazolo[1,5-a][1,3,5]triazin-4-amine (Intermediate 2, 120 mg, 311 μmol) and 2-methyl-1-(methylamino)propan-2-ol (96.4 mg, 934 μmol, CAS 67622-86-0) were dissolved in acetonitrile (2.9 mL), N,N-diisopropylethylamine (270 μL, 1.6 mmol) was added and the mixture was stirred for 16 h at 70° C. The mixture was purified by preparative HPLC (HT basic) to give 27.2 mg (21% yield) of the title compound.

LC-MS (Method 1): Rₜ=0.94 min; MS (ESIneg): m/z=407 [M−H]⁻

¹H-NMR (400 MHz, DMSO-d6) δ[ppm]: 0.790 (5.64), 1.083 (2.34), 1.243 (15.86), 1.260 (15.71), 1.301 (0.49), 2.336 (0.55), 2.518 (8.22), 2.522 (5.29), 2.539 (4.77), 2.939 (0.78), 3.095 (4.02), 3.412 (1.94), 3.531 (0.78), 4.324 (0.95), 4.798 (1.71), 4.875 (0.78), 7.106 (3.10), 7.114 (3.44), 7.121 (3.50), 7.395 (1.48), 7.506 (1.45), 7.758 (16.00), 8.213 (0.46), 8.735 (0.81), 12.229 (1.74).

### Example 6

### N-[(1H-benzimidazol-2-yl)methyl]-2-[(3S)-3-methylmorpholin-4-yl]-8-(propan-2-yl)pyrazolo[1,5-a][1,3,5]triazin-4-amine

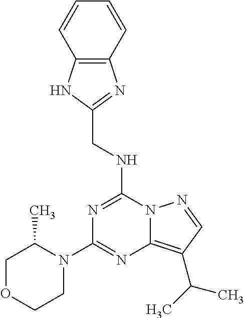 [SMILES: CC(C)c1cnn2c(NCc3nc4ccccc4[nH]3)nc(N3CCOC[C@@H]3C)nc12]

N-[(1H-benzimidazol-2-yl)methyl]-2-(methanesulfonyl)-8-(propan-2-yl)pyrazolo[1,5-a][1,3,5]triazin-4-amine (Intermediate 2, 120 mg, 311 μmol) and (3S)-3-methylmorpholine (94.5 mg, 934 μmol, CAS 350595-57-2) were dissolved in acetonitrile (2.9 mL), N,N-diisopropylethylamine (270 μL, 1.6 mmol) was added and the mixture was stirred for 16 h at 70° C. N,N-diisopropylethylamine (270 μL, 1.6 mmol) was added and the mixture was stirred for 1 h at 130° C. and for 4 h at 150° C. in the microwave. The mixture was purified by preparative HPLC to give 10.1 mg (8% yield) of the title compound.

LC-MS (Method 1): Rₜ=1.01 min; MS (ESIneg): m/z=405 [M−H]⁻

¹H-NMR (400 MHz, DMSO-d6) δ[ppm]: 0.919 (2.43), 1.241 (16.00), 1.258 (15.85), 1.299 (0.93), 1.316 (0.89), 2.327 (0.81), 2.408 (0.78), 2.669 (0.82), 2.911 (0.47), 2.928 (1.17), 2.945 (1.88), 2.962 (1.28), 2.979 (1.36), 3.012 (0.68), 3.020 (0.58), 3.244 (0.61), 3.269 (1.04), 3.304 (0.68), 3.411 (0.90), 3.417 (0.90), 3.440 (1.35), 3.546 (1.71), 3.575 (1.17), 3.770 (0.99), 3.797 (0.92), 4.099 (1.21), 4.132 (1.17), 4.452 (0.86), 4.824 (1.96), 4.834 (2.61), 4.847 (2.00), 7.105 (2.32), 7.113 (2.49), 7.120 (2.61), 7.127 (2.53), 7.473 (0.61), 7.797 (5.78), 8.850 (0.88), 8.864 (1.70), 8.878 (0.88), 12.250 (1.32).

### Example 7

### N-[(1H-benzimidazol-2-yl)methyl]-2-[(3R)-3-methylmorpholin-4-yl]-8-(propan-2-yl)pyrazolo[1,5-a][1,3,5]triazin-4-amine

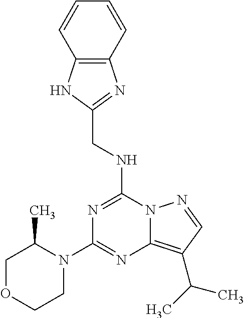 [SMILES: CC(C)c1cnn2c(NCc3nc4ccccc4[nH]3)nc(N3CCOC[C@H]3C)nc12]

N-[(1H-benzimidazol-2-yl)methyl]-2-(methanesulfonyl)-8-(propan-2-yl)pyrazolo[1,5-a][1,3,5]triazin-4-amine (Intermediate 2, 120 mg, 311 μmol) and (3R)-3-methylmorpholine (94.5 mg, 934 μmol, CAS 74572-04-6) were dissolved in acetonitrile (2.9 mL), N,N-diisopropylethylamine (270 μL, 1.6 mmol) was added and the mixture was stirred for 16 h at 70° C. N,N-diisopropylethylamine (270 μL, 1.6 mmol) was added and the mixture was stirred for 1 h at 130° C. and for 4 h at 150° C. in the microwave. The mixture was purified by preparative HPLC to give 11.3 mg (9% yield) of the title compound.

LC-MS (Method 1): Rₜ=1.01 min; MS (ESIneg): m/z=405 [M−H]⁻

¹H-NMR (400 MHz, DMSO-d6) δ[ppm]: 0.929 (2.49), 1.241 (15.88), 1.258 (16.00), 1.298 (0.85), 1.316 (0.77), 2.327 (1.20), 2.408 (0.70), 2.669 (1.20), 2.910 (0.50), 2.927 (1.18), 2.945 (1.89), 2.962 (1.28), 2.979 (1.39), 3.012 (0.68), 3.020 (0.58), 3.244 (0.62), 3.270 (1.08), 3.304 (0.81), 3.417 (0.93), 3.439 (1.35), 3.546 (1.76), 3.575 (1.18), 3.776 (1.02), 3.797 (0.93), 4.100 (1.24), 4.132 (1.18), 4.452 (0.89), 4.823 (2.03), 4.833 (2.69), 4.846 (2.05), 7.085 (0.48), 7.099 (1.74), 7.109 (2.09), 7.115 (2.67), 7.122 (2.26), 7.127 (1.89), 7.145 (0.62), 7.381 (1.58), 7.398 (1.47), 7.510 (1.49), 7.530 (1.41), 7.797 (5.70), 8.849 (0.91), 8.863 (1.76), 8.877 (0.89), 12.240 (2.18).

### Example 8

### N-[(1H-benzimidazol-2-yl)methyl]-2-(1,4-oxazepan-4-yl)-8-(propan-2-yl)pyrazolo[1,5-a][1,3,5]triazin-4-amine

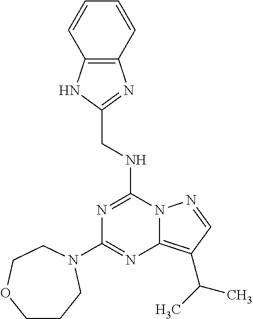 [SMILES: CC(C)c1cnn2c(NCc3nc4ccccc4[nH]3)nc(N3CCCOCC3)nc12]

N-[(1H-benzimidazol-2-yl)methyl]-2-(methanesulfonyl)-8-(propan-2-yl)pyrazolo[1,5-a][1,3,5]triazin-4-amine (Intermediate 2, 120 mg, 311 μmol) and 1,4-oxazepane hydrochloride (129 mg, 934 μmol, CAS 178312-62-4) were dissolved in acetonitrile (2.9 mL), N,N-diisopropylethylamine (270 μL, 1.6 mmol) was added and the mixture was stirred for 16 h at 70° C. The mixture was purified by preparative HPLC (HT acidic) to give 65.0 mg (97% purity, 50% yield) of the title compound.

LC-MS (Method 1): Rₜ=0.97 min; MS (ESIneg): m/z=405 [M−H]⁻

¹H-NMR (400 MHz, DMSO-d6) δ[ppm]: 1.241 (16.00), 1.258 (15.82), 1.415 (0.50), 1.823 (0.54), 2.326 (0.84), 2.668 (0.84), 2.902 (0.50), 2.919 (1.16), 2.936 (1.51), 2.953 (1.09), 2.971 (0.43), 3.236 (0.64), 3.454 (0.99), 3.663 (2.20), 4.811 (2.48), 7.105 (2.30), 7.112 (2.76), 7.119 (2.50), 7.141 (0.57), 7.376 (1.52), 7.393 (1.38), 7.505 (1.59), 7.524 (1.45), 7.769 (6.08), 8.808 (1.02), 8.822 (1.93), 8.836 (0.98), 12.234 (2.18).

### Example 9

### N-[(1H-benzimidazol-2-yl)methyl]-2-(2-oxa-6-azaspiro[3.3]heptan-6-yl)-8-(propan-2-yl)pyrazolo[1,5-a][1,3,5]triazin-4-amine

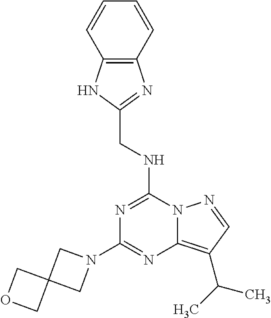 [SMILES: CC(C)c1cnn2c(NCc3nc4ccccc4[nH]3)nc(N3CC4(COC4)C3)nc12]

N-[(1H-benzimidazol-2-yl)methyl]-2-(methanesulfonyl)-8-(propan-2-yl)pyrazolo[1,5-a][1,3,5]triazin-4-amine (Intermediate 2, 120 mg, 311 μmol) and 2-oxa-6-azaspiro[3.3]heptane oxalic acid salt (177 mg, 934 μmol, CAS 1159599-99-1) were dissolved in acetonitrile (2.9 mL), N,N-diisopropylethylamine (270 μL, 1.6 mmol) was added and the mixture was stirred for 16 h at 70° C. N,N-diisopropylethylamine (270 μL, 1.6 mmol) was added and the mixture was stirred for 1 h at 130° C. in the microwave. The mixture was evaporated and stirred with DMSO. The precipitate was collected by filtration, washed with water and dried under reduced pressure to give 24.7 mg (19% yield) of the title compound.

LC-MS (Method 1): Rₜ=0.81 min; MS (ESIneg): m/z=403 [M−H]⁻

¹H-NMR (400 MHz, DMSO-d6) δ[ppm]: 1.229 (16.00), 1.246 (15.81), 2.074 (0.44), 2.322 (0.73), 2.326 (0.92), 2.668 (0.92), 2.918 (0.46), 2.935 (1.11), 2.952 (1.44), 2.969 (1.05), 2.987 (0.44), 3.159 (0.84), 3.171 (0.84), 4.080 (10.83), 4.634 (12.75), 4.843 (3.35), 4.859 (3.29), 7.101 (0.46), 7.116 (1.51), 7.127 (1.90), 7.132 (2.30), 7.138 (1.94), 7.146 (1.44), 7.150 (1.49), 7.163 (0.46), 7.408 (1.43), 7.424 (1.25), 7.528 (1.30), 7.547 (1.21), 7.806 (6.02), 8.825 (0.86), 8.840 (1.71), 8.854 (0.81), 12.203 (1.87).

### Example 10

### 1-{[4-{[(1H-benzimidazol-2-yl)methyl]amino}-8-(propan-2-yl)pyrazolo[1,5-a][1,3,5]triazin-2-yl]amino}-2-methylpropan-2-ol

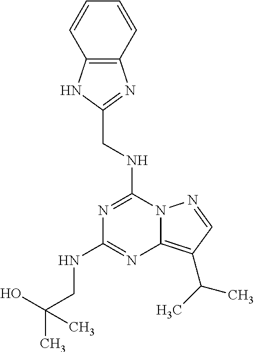 [SMILES: CC(C)c1cnn2c(NCc3nc4ccccc4[nH]3)nc(NCC(C)(C)O)nc12]

N-[(1H-benzimidazol-2-yl)methyl]-2-(methanesulfonyl)-8-(propan-2-yl)pyrazolo[1,5-a][1,3,5]triazin-4-amine (Intermediate 2, 120 mg, 311 μmol) and 1-amino-2-methylpropan-2-ol (83.3 mg, 934 μmol, CAS 2854-16-2) were dissolved in acetonitrile (2.9 mL), N,N-diisopropylethylamine (270 μL, 1.6 mmol) was added and the mixture was stirred for 16 h at 70° C. The mixture was purified by preparative HPLC (HT acidic) to give 84.7 mg (96% purity, 66% yield) of the title compound.

LC-MS (Method 1): Rₜ=0.79 min; MS (ESIneg): m/z=393 [M−H]⁻

¹H-NMR (400 MHz, DMSO-d6) δ[ppm]: 0.866 (1.42), 1.088 (8.28), 1.136 (1.73), 1.232 (16.00), 1.249 (15.05), 2.322 (0.73), 2.327 (0.81), 2.539 (2.85), 2.669 (0.87), 2.892 (0.76), 2.908 (1.81), 2.926 (2.32), 2.943 (1.69), 2.960 (0.66), 3.235 (2.47), 4.842 (4.48), 4.856 (4.27), 6.745 (1.10), 7.132 (3.51), 7.413 (1.23), 7.526 (1.25), 7.750 (7.47), 8.157 (1.64), 8.705 (1.00), 12.236 (1.07).

### Example 11

### (rac)-N-[(1H-benzimidazol-2-yl)methyl]-2-[2-methylmorpholin-4-yl]-8-(propan-2-yl)pyrazolo[1,5-a][1,3,5]triazin-4-amine

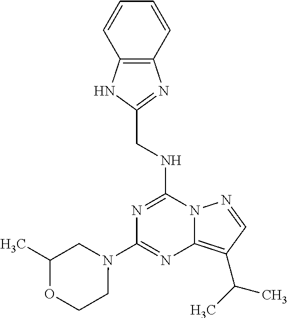 [SMILES: CC1CN(c2nc(NCc3nc4ccccc4[nH]3)n3ncc(C(C)C)c3n2)CCO1]

N-[(1H-benzimidazol-2-yl)methyl]-2-(methanesulfonyl)-8-(propan-2-yl)pyrazolo[1,5-a][1,3,5]triazin-4-amine (Intermediate 2, 240 mg, 623 μmol) and 2-methylmorpholine (189 mg, 1.87 mmol, CAS 27550-90-9) were dissolved in acetonitrile (5.9 mL), N,N-diisopropylethylamine (540 μL, 3.1 mmol) was added and the mixture was stirred for 16 h at 70° C. The mixture was purified by preparative HPLC (HT acidic) to give 123 mg (99% purity, 48% yield) of the title compound.

LC-MS (Method 1): Rₜ=1.00 min; MS (ESIneg): m/z=405 [M−H]⁻

¹H-NMR (400 MHz, DMSO-d6) δ[ppm]: 0.993 (2.03), 1.006 (2.01), 1.236 (15.78), 1.254 (16.00), 2.401 (0.40), 2.428 (0.63), 2.460 (0.56), 2.518 (1.89), 2.522 (1.27), 2.539 (1.99), 2.781 (0.65), 2.805 (0.43), 2.917 (0.46), 2.935 (1.13), 2.952 (1.51), 2.969 (1.08), 3.268 (0.64), 3.295 (1.20), 3.716 (0.60), 3.742 (0.54), 4.266 (1.05), 4.299 (1.04), 4.836 (3.01), 4.849 (2.99), 7.114 (1.57), 7.128 (1.70), 7.388 (0.59), 7.403 (0.59), 7.514 (0.61), 7.802 (7.33), 8.153 (0.99), 8.856 (0.75), 8.870 (1.60), 8.885 (0.72), 12.248 (0.79).

### Example 12

### N-[(1H-benzimidazol-2-yl)methyl]-2-(2,2-dimethylmorpholin-4-yl)-8-(propan-2-yl)pyrazolo[1,5-a][1,3,5]triazin-4-amine

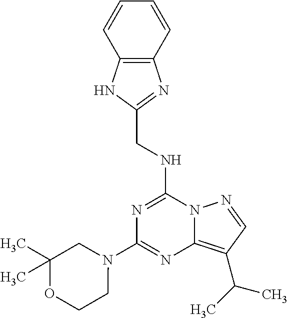 [SMILES: CC(C)c1cnn2c(NCc3nc4ccccc4[nH]3)nc(N3CCOC(C)(C)C3)nc12]

N-[(1H-benzimidazol-2-yl)methyl]-2-(methanesulfonyl)-8-(propan-2-yl)pyrazolo[1,5-a][1,3,5]triazin-4-amine (Intermediate 2, 120 mg, 311 μmol) and 2,2-dimethylmorpholine (108 mg, 934 μmol) were dissolved in acetonitrile (2.9 mL), N,N-diisopropylethylamine (270 μL, 1.6 mmol) was added and the mixture was stirred for 16 h at 70° C. The mixture was stirred in dichloromethane, filtered, washed with dichloromethane to give 29.7 mg (22% yield) of the title compound.

LC-MS (Method 1): Rₜ=1.01 min; MS (ESIneg): m/z=419 [M−H]⁻

¹H-NMR (400 MHz, DMSO-d6) δ[ppm]: 0.871 (1.49), 1.231 (15.50), 1.249 (16.00), 1.332 (0.55), 1.349 (0.55), 2.327 (0.42), 2.522 (1.99), 2.669 (0.45), 2.904 (0.42), 2.921 (1.06), 2.938 (1.41), 2.955 (1.01), 3.238 (0.63), 3.373 (1.50), 3.474 (1.34), 3.564 (1.79), 4.798 (2.14), 4.812 (2.13), 5.759 (0.47), 7.086 (0.46), 7.099 (1.47), 7.103 (1.49), 7.108 (1.68), 7.115 (2.65), 7.122 (1.89), 7.127 (1.63), 7.131 (1.63), 7.144 (0.59), 7.372 (1.33), 7.379 (0.85), 7.388 (1.18), 7.393 (1.08), 7.505 (1.21), 7.511 (1.21), 7.521 (0.78), 7.527 (1.19), 7.788 (5.98), 8.859 (0.71), 8.873 (1.46), 8.887 (0.70), 12.258 (1.88).

### Example 13

### N⁴-[(1H-benzimidazol-2-yl)methyl]-N²-phenyl-8-(propan-2-yl)pyrazolo[1,5-a][1,3,5]triazine-2,4-diamine

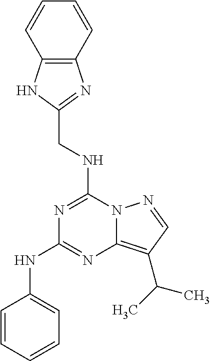 [SMILES: CC(C)c1cnn2c(NCc3nc4ccccc4[nH]3)nc(Nc3ccccc3)nc12]

N-[(1H-benzimidazol-2-yl)methyl]-2-(methanesulfonyl)-8-(propan-2-yl)pyrazolo[1,5-a][1,3,5]triazin-4-amine (Intermediate 2, 100 mg, 259 μmol) and aniline (36.2 mg, 389 μmol) were dissolved in N-methylpyrrolidone (2.0 mL) and 4-methylbenzenesulfonic acid (4.47 mg, 25.9 μmol) was added. The mixture was stirred for 2 h at 180° C. in the microwave. The mixture was purified by preparative HPLC to give 11.2 mg (10% yield) of the title compound.

LC-MS (Method 1): Rₜ=1.16 min; MS (ESIpos): m/z=399 [M+H]⁺

¹H-NMR (400 MHz, DMSO-d6) δ[ppm]: 0.968 (0.50), 1.108 (2.72), 1.145 (0.45), 1.209 (0.53), 1.231 (0.55), 1.313 (15.54), 1.331 (16.00), 2.518 (2.74), 2.523 (1.94), 3.029 (1.02), 3.046 (1.37), 3.063 (0.97), 4.926 (2.36), 4.941 (2.35), 6.852 (0.62), 6.870 (1.35), 6.888 (0.78), 7.118 (1.53), 7.124 (1.95), 7.133 (2.47), 7.141 (2.46), 7.148 (2.51), 7.166 (1.75), 7.185 (0.87), 7.405 (1.15), 7.413 (0.70), 7.421 (0.96), 7.427 (0.94), 7.546 (1.06), 7.551 (0.99), 7.559 (0.62), 7.568 (1.00), 7.720 (0.93), 7.738 (0.89), 7.889 (6.51), 8.957 (0.52), 8.972 (1.05), 8.987 (0.50), 9.308 (3.03), 12.295 (1.64).

### Example 14

### N⁴-[(1H-benzimidazol-2-yl)methyl]-8-(propan-2-yl)-N²-(pyridin-4-yl)pyrazolo[1,5-a][1,3,5]triazine-2,4-diamine

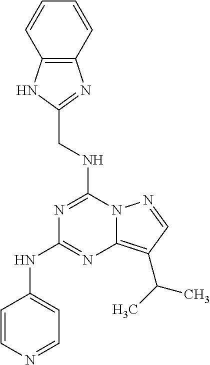 [SMILES: CC(C)c1cnn2c(NCc3nc4ccccc4[nH]3)nc(Nc3ccncc3)nc12]

N-[(1H-benzimidazol-2-yl)methyl]-2-(methanesulfonyl)-8-(propan-2-yl)pyrazolo[1,5-a][1,3,5]triazin-4-amine (Intermediate 2, 100 mg, 259 μmol) and pyridin-4-amine (97.7 mg, 1.04 mmol) were dissolved in N-methylpyrrolidone (3.0 mL) and 4-methylbenzenesulfonic acid (4.47 mg, 25.9 μmol) was added. The mixture was stirred for 2 h at 160° C. in the microwave. The mixture was purified by preparative HPLC to give 13.5 mg (13% yield) of the title compound.

LC-MS (Method 1): Rₜ=0.75 min; MS (ESIneg): m/z=398 [M−H]⁻

¹H-NMR (400 MHz, DMSO-d6) δ[ppm]: 1.107 (6.76), 1.312 (0.42), 1.327 (15.41), 1.344 (16.00), 2.518 (2.40), 2.523 (1.70), 3.053 (0.40), 3.070 (1.02), 3.087 (1.33), 3.105 (0.95), 4.946 (2.28), 4.961 (2.28), 7.122 (1.97), 7.129 (1.93), 7.137 (2.02), 7.145 (2.17), 7.155 (0.45), 7.665 (1.71), 7.679 (1.76), 7.970 (6.28), 8.192 (1.01), 8.212 (2.24), 8.227 (2.09), 9.162 (0.53), 9.178 (1.09), 9.192 (0.52), 9.761 (2.85).

### Example 15

### N-[(1H-benzimidazol-2-yl)methyl]-8-chloro-2-(morpholin-4-yl)pyrazolo[1,5-a][1,3,5]triazin-4-amine

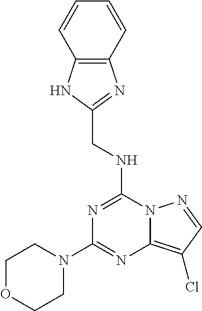 [SMILES: Clc1cnn2c(NCc3nc4ccccc4[nH]3)nc(N3CCOCC3)nc12]

N-[(1H-benzimidazol-2-yl)methyl]-8-chloro-2-(methanesulfonyl)pyrazolo[1,5-a][1,3,5]triazin-4-amine (Intermediate 12, 100 mg, 265 μmol) and morpholine (69 μL, 790 μmol; CAS 110-91-8) were dissolved in acetonitrile (2.5 mL), N,N-diisopropylethylamine (140 μL, 790 μmol) was added and the mixture was stirred overnight at 70° C. The mixture was purified by flash chromatography (ethyl acetate/ethanol gradient 0-25% ethanol) and preparative HPLC (HT basic) to give 19.1 mg (99% purity, 19% yield) of the title compound.

LC-MS (Method 2): Rₜ=1.00 min; MS (ESIpos): m/z=385 [M+H]⁺

¹H-NMR (400 MHz, DMSO-d6) δ[ppm]: 2.322 (0.87), 2.327 (1.19), 2.331 (0.87), 2.518 (5.45), 2.522 (3.35), 2.664 (0.87), 2.669 (1.21), 2.673 (0.87), 2.678 (0.42), 3.166 (0.56), 3.488 (3.81), 3.618 (5.87), 4.871 (10.76), 7.109 (0.75), 7.119 (4.08), 7.127 (3.95), 7.134 (4.10), 7.142 (4.60), 7.152 (0.92), 7.470 (1.12), 8.051 (16.00).

### Example 16

### (rac)-1-(4-{[(1H-benzimidazol-2-yl)methyl]amino}-8-bromopyrazolo[1,5-a][1,3,5]triazin-2-yl)piperidin-3-ol

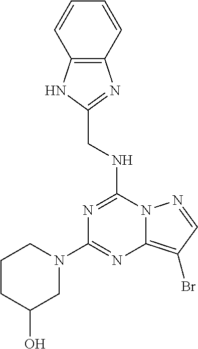 [SMILES: OC1CCCN(c2nc(NCc3nc4ccccc4[nH]3)n3ncc(Br)c3n2)C1]

N-[(1H-benzimidazol-2-yl)methyl]-8-bromo-2-(methanesulfonyl)pyrazolo[1,5-a][1,3,5]triazin-4-amine (Intermediate 9, 110 mg, 260 μmol) and piperidin-3-ol (79.0 mg, 781 μmol) were dissolved in acetonitrile (2.5 mL), N,N-diisopropylethylamine (140 μL, 780 μmol) was added and the mixture was stirred overnight at 70° C. Ethanol was added, the precipitate was collected by filtration, washed with water and ethanol to give 60.5 mg (52% yield) of the title compound.

LC-MS (Method 1): Rₜ=0.81 min; MS (ESIpos): m/z=443 [M+H]⁺

¹H-NMR (500 MHz, DMSO-d6) δ[ppm]: 1.365 (0.51), 1.388 (0.42), 1.836 (0.45), 2.049 (1.78), 2.361 (0.48), 2.635 (0.49), 2.965 (0.48), 3.075 (16.00), 3.470 (0.53), 4.132 (0.58), 4.159 (0.54), 4.325 (0.44), 4.350 (0.43), 4.593 (0.69), 4.909 (2.87), 7.132 (1.16), 7.141 (1.23), 7.432 (0.43), 7.543 (0.44), 7.930 (3.26).

### Example 17

### 1-(4-{[(1H-benzimidazol-2-yl)methyl]amino}-8-bromopyrazolo[1,5-a][1,3,5]triazin-2-yl)piperidin-4-ol

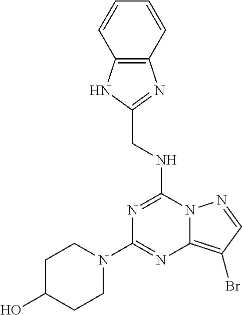 [SMILES: OC1CCN(c2nc(NCc3nc4ccccc4[nH]3)n3ncc(Br)c3n2)CC1]

N-[(1H-benzimidazol-2-yl)methyl]-8-bromo-2-(methanesulfonyl)pyrazolo[1,5-a][1,3,5]triazin-4-amine (Intermediate 9, 110 mg, 260 μmol) and piperidin-4-ol (79.0 mg, 781 μmol) were dissolved in acetonitrile (2.5 mL), N,N-diisopropylethylamine (140 μL, 780 μmol) was added and the mixture was stirred overnight at 70° C. The mixture was diluted with ethanol, filtered, washed with water and ethanol to give 42.5 mg (36% yield) of the title compound.

LC-MS (Method 1): Rₜ=0.75 min; MS (ESIpos): m/z=443 [M+H]⁺

¹H-NMR (400 MHz, DMSO-d6) δ[ppm]: 1.179 (0.97), 1.232 (1.71), 1.609 (0.93), 2.074 (3.38), 2.331 (1.63), 2.518 (10.33), 2.522 (6.37), 2.669 (2.25), 2.673 (1.63), 3.184 (1.71), 3.619 (1.20), 3.627 (1.20), 4.107 (1.32), 4.662 (2.49), 4.672 (2.49), 4.851 (8.54), 7.110 (2.06), 7.119 (3.07), 7.132 (3.30), 7.141 (2.21), 7.388 (1.48), 7.405 (1.40), 7.523 (1.51), 7.541 (1.28), 8.001 (16.00), 12.250 (1.86).

### Example 18

### N-[(1H-benzimidazol-2-yl)methyl]-8-bromo-2-(4-methylpiperazin-1-yl)pyrazolo[1,5-a][1,3,5]triazin-4-amine

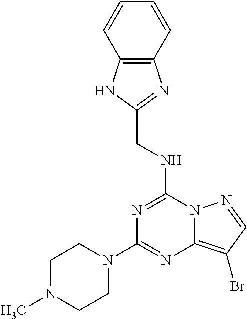 [SMILES: CN1CCN(c2nc(NCc3nc4ccccc4[nH]3)n3ncc(Br)c3n2)CC1]

N-[(1H-benzimidazol-2-yl)methyl]-8-bromo-2-(methanesulfonyl)pyrazolo[1,5-a][1,3,5]triazin-4-amine (Intermediate 9, 110 mg, 260 μmol) and 1-methylpiperazine (78.3 mg, 781 μmol) were dissolved in acetonitrile (2.5 mL), N,N-diisopropylethylamine (140 μL, 780 μmol) was added and the mixture was stirred overnight at 70° C. The mixture was purified by preparative HPLC (HT basic) to give 55.0 mg (45% yield) of the title compound.

LC-MS (Method 2): Rₜ=1.00 min; MS (ESIpos): m/z=442 [M+H]⁺

¹H-NMR (400 MHz, DMSO-d6) δ[ppm]: 0.843 (2.06), 0.861 (4.98), 0.879 (2.69), 1.262 (0.58), 1.279 (0.97), 1.300 (1.05), 1.317 (0.82), 1.353 (0.55), 1.370 (1.25), 1.387 (1.05), 1.404 (0.70), 1.907 (1.05), 2.163 (6.19), 2.241 (2.26), 2.322 (2.30), 2.326 (2.92), 2.331 (2.30), 2.382 (0.43), 2.522 (13.08), 2.539 (3.62), 2.664 (1.71), 2.668 (2.34), 2.673 (1.79), 2.686 (0.74), 2.692 (0.82), 2.729 (2.18), 2.888 (2.61), 3.371 (2.14), 3.384 (1.79), 3.399 (0.90), 3.643 (3.35), 4.321 (0.74), 4.859 (7.47), 4.874 (7.55), 7.123 (4.05), 7.136 (4.44), 7.394 (1.67), 7.409 (1.71), 7.525 (1.75), 8.025 (16.00), 9.091 (1.44), 9.104 (2.80), 9.119 (1.56), 12.254 (2.57).

### Example 19

### 8-bromo-N-[(3H-imidazo[4,5-b]pyridin-2-yl)methyl]-2-(piperazin-1-yl)pyrazolo[1,5-a][1,3,5]triazin-4-amine formic acid salt

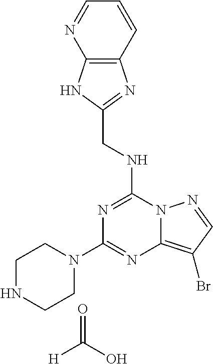

8-bromo-N-[(3H-imidazo[4,5-b]pyridin-2-yl)methyl]-2-(methanesulfonyl)pyrazolo[1,5-a][1,3,5]triazin-4-amine (Intermediate 14, 80.0 mg, 189 μmol) and piperazine (48.8 mg, 567 μmol) were dissolved in acetonitrile (1.8 mL), N,N-diisopropylethylamine (99 μL, 570 μmol) was added and the mixture was stirred overnight at 70° C. The mixture was purified by preparative HPLC twice (1. HT basic, 2. see below) to give 16.0 mg (95% purity, 17% yield) of the title compound.

### Preparative HPLC Method

Instrument: Waters Autopurificationsystem; Column: XBrigde C18 5μ, 100×30 mm; eluent A: water+0.1 vol % formic acid; eluent B: methanol; gradient: 0.0-0.50 min 10% B (40-70 mL/min), 0.51-8.50 min 10-45% B; flow: 70 mL/min; temperature: 25° C.; DAD scan: 210-400 nm.

### Analytical HPLC Method

Instrument: Waters Acquity UPLCMS SingleQuad; Column: Acquity UPLC BEH C18 1.7μ, 50×2.1 mm; eluent A: water+0.1 vol % formic acid; eluent B: methanol; gradient: 0-1.6 min 1-99% B, 1.6-2.0 min 99% B; flow: 0.8 mL/min; temperature: 60° C.; DAD scan: 210-400 nm.

Analytical HPLC: Rₜ=0.76 min; MS (ESIpos): m/z=427 [M−H]⁻

¹H-NMR (400 MHz, METHANOL-d4) δ [ppm]: 0.083 (1.56), 0.088 (5.20), 0.098 (10.28), 0.112 (1.82), 0.874 (1.43), 0.902 (2.21), 0.917 (1.30), 1.028 (1.17), 1.290 (6.76), 1.401 (8.33), 3.014 (4.68), 3.349 (2.34), 3.661 (7.28), 3.905 (7.41), 4.611 (2.34), 5.026 (15.09), 7.276 (2.34), 7.288 (2.73), 7.296 (2.73), 7.308 (2.47), 7.812 (1.17), 7.872 (16.00), 7.927 (1.56), 7.944 (1.56), 8.345 (2.34), 8.490 (1.56).

### Example 20

### 8-bromo-N-[(7-methyl-3H-imidazo[4,5-b]pyridin-2-yl)methyl]-2-(morpholin-4-yl)pyrazolo[1,5-a][1,3,5]triazin-4-amine

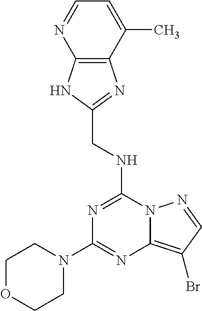 [SMILES: Cc1ccnc2[nH]c(CNc3nc(N4CCOCC4)nc4c(Br)cnn34)nc12]

8-bromo-2-(methanesulfonyl)-N-[(7-methyl-3H-imidazo[4,5-b]pyridin-2-yl)methyl]pyrazolo[1,5-a][1,3,5]triazin-4-amine (Intermediate 16, 88.0 mg, 201 μmol) and morpholine (53 μL, 600 μmol; CAS 110-91-8) were dissolved in acetonitrile (1.9 mL), N,N-diisopropylethylamine (110 μL, 600 μmol) was added and the mixture was stirred overnight at 70° C. The mixture was purified by preparative HPLC (HT basic) followed by precipitation from ethanol to give 27.5 mg (30% yield) of the title compound.

LC-MS (Method 2): Rₜ=0.85 min; MS (ESIpos): m/z=444 [M+H]⁺

¹H-NMR (400 MHz, DMSO-d6) δ[ppm]: 1.035 (2.13), 1.052 (3.99), 1.070 (2.18), 2.322 (2.23), 2.326 (3.03), 2.331 (2.23), 2.522 (9.89), 2.664 (2.29), 2.668 (3.03), 2.673 (2.23), 3.429 (1.81), 3.471 (4.73), 3.604 (7.34), 4.876 (12.65), 6.997 (4.73), 7.010 (4.89), 8.031 (16.00), 8.112 (2.55), 8.123 (2.55).

### Example 21

### 8-bromo-2-(morpholin-4-yl)-N-[(4,5,6,7-tetrahydro-1H-benzimidazol-2-yl)methyl]pyrazolo[1,5-a][1,3,5]triazin-4-amine

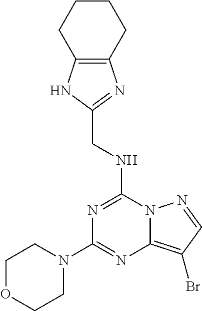 [SMILES: Brc1cnn2c(NCc3nc4c([nH]3)CCCC4)nc(N3CCOCC3)nc12]

8-bromo-2-(methanesulfonyl)-N-[(4,5,6,7-tetrahydro-1H-benzimidazol-2-yl)methyl]pyrazolo[1,5-a][1,3,5]triazin-4-amine (Intermediate 18, 80.0 mg, 188 μmol) and morpholine (49 μL, 560 μmol; CAS 110-91-8) were dissolved in acetonitrile (1.8 mL), N,N-diisopropylethylamine (98 μL, 560 μmol) was added and the mixture was stirred overnight at 70° C. The mixture was purified by preparative HPLC (HT basic) to give 30.0 mg (99% purity, 37% yield) of the title compound.

LC-MS (Method 2): Rₜ=1.04 min; MS (ESIpos): m/z=433 [M+H]⁺

¹H-NMR (400 MHz, DMSO-d6) δ[ppm]: 1.681 (12.57), 1.903 (0.47), 2.327 (2.27), 2.331 (1.87), 2.539 (1.97), 2.669 (2.03), 3.166 (0.60), 3.608 (12.17), 3.620 (10.57), 3.692 (11.43), 3.705 (12.87), 3.715 (7.23), 4.577 (6.50), 4.586 (6.47), 7.987 (16.00), 8.810 (2.33), 11.316 (1.33).

### Example 22

### 8-bromo-N-[(3H-imidazo[4,5-c]pyridin-2-yl)methyl]-2-(morpholin-4-yl)pyrazolo[1,5-a][1,3,5]triazin-4-amine

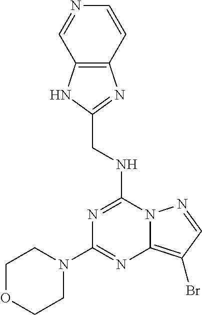 [SMILES: Brc1cnn2c(NCc3nc4ccncc4[nH]3)nc(N3CCOCC3)nc12]

8-bromo-N-[(3H-imidazo[4,5-c]pyridin-2-yl)methyl]-2-(methanesulfonyl)pyrazolo[1,5-a][1,3,5]triazin-4-amine (Intermediate 20, 80.0 mg, 189 μmol) and morpholine (49 μL, 570 μmol; CAS 110-91-8) were dissolved in acetonitrile (1.8 mL), N,N-diisopropylethylamine (99 μL, 570 μmol) was added and the mixture was stirred overnight at 70° C. The mixture was purified by preparative HPLC (HT basic) to give 10.0 mg (90% purity, 11% yield) of the title compound.

LC-MS (Method 2): Rₜ=0.75 min; MS (ESIpos): m/z=430 [M+H]⁺

¹H-NMR (400 MHz, DMSO-d6) δ[ppm]: 1.903 (2.13), 2.075 (9.93), 2.332 (3.23), 2.336 (1.34), 2.518 (16.00), 2.523 (11.59), 2.540 (0.87), 2.673 (3.23), 2.678 (1.34), 2.687 (0.39), 2.729 (1.73), 2.889 (2.21), 3.165 (4.33), 3.468 (1.97), 3.591 (2.44), 4.910 (5.44), 7.478 (0.47), 8.046 (8.51), 8.247 (3.31), 8.260 (3.15), 8.810 (0.63).

### Example 23

### 8-bromo-2-(morpholin-4-yl)-N-[(5-phenyl-1H-imidazol-2-yl)methyl]pyrazolo[1,5-a][1,3,5]triazin-4-amine

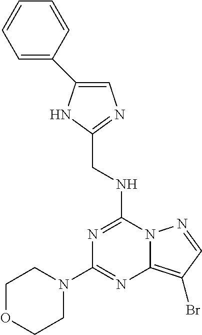 [SMILES: Brc1cnn2c(NCc3ncc(-c4ccccc4)[nH]3)nc(N3CCOCC3)nc12]

8-bromo-2-(methanesulfonyl)-N-[(5-phenyl-1H-imidazol-2-yl)methyl]pyrazolo[1,5-a][1,3,5]triazin-4-amine (Intermediate 22, 130 mg, 290 μmol) and morpholine (76 μL, 870 μmol; CAS 110-91-8) were dissolved in acetonitrile (2.7 mL), N,N-diisopropylethylamine (150 μL, 870 μmol) was added and the mixture was stirred overnight at 70° C. The mixture was purified by preparative HPLC (HT basic) to give 28.0 mg (99% purity, 21% yield) of the title compound.

LC-MS (Method 3): Rₜ=1.08 min; MS (ESIpos): m/z=455 [M+H]⁺

¹H-NMR (400 MHz, DMSO-d6) δ[ppm]: 2.326 (3.87), 2.331 (2.87), 2.336 (1.33), 2.518 (15.47), 2.522 (9.73), 2.539 (0.93), 2.669 (3.93), 2.673 (2.87), 3.567 (5.47), 3.677 (7.00), 3.690 (7.47), 3.700 (4.00), 4.701 (4.13), 4.714 (3.73), 7.134 (1.20), 7.153 (2.80), 7.171 (1.73), 7.217 (0.47), 7.261 (0.80), 7.295 (3.47), 7.315 (5.47), 7.334 (2.93), 7.363 (0.60), 7.383 (0.87), 7.402 (0.47), 7.516 (4.27), 7.521 (4.27), 7.592 (0.87), 7.610 (0.73), 7.721 (5.07), 7.738 (4.67), 7.742 (3.73), 8.009 (16.00), 8.995 (1.47), 11.935 (1.60).

### Example 24

### 8-bromo-2-(morpholin-4-yl)-N-[(5-phenyl-4H-1,2,4-triazol-3-yl)methyl]pyrazolo[1,5-a][1,3,5]triazin-4-amine

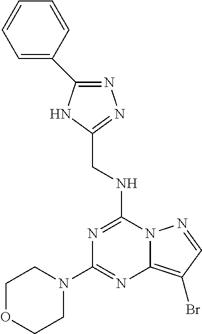 [SMILES: Brc1cnn2c(NCc3nnc(-c4ccccc4)[nH]3)nc(N3CCOCC3)nc12]

8-bromo-2-(methanesulfonyl)-N-[(5-phenyl-4H-1,2,4-triazol-3-yl)methyl]pyrazolo[1,5-a][1,3,5]triazin-4-amine (Intermediate 24, 90.0 mg, 200 μmol) and morpholine (52 μL, 600 μmol; CAS 110-91-8) were dissolved in acetonitrile (1.9 mL), N,N-diisopropylethylamine (100 μL, 600 μmol) was added and the mixture was stirred overnight at 70° C. The mixture was purified by preparative HPLC (HT basic) to give 10.5 mg (99% purity, 11% yield) of the title compound.

LC-MS (Method 3): Rₜ=0.79 min; MS (ESIpos): m/z=456 [M+H]⁺

¹H-NMR (400 MHz, DMSO-d6) δ[ppm]: 2.074 (1.03), 2.331 (1.98), 2.336 (0.90), 2.518 (10.47), 2.523 (6.38), 2.539 (2.65), 2.673 (1.98), 2.678 (0.90), 3.544 (5.44), 3.652 (5.75), 3.664 (6.83), 4.767 (3.33), 7.422 (1.35), 7.440 (1.89), 7.452 (3.01), 7.471 (3.42), 7.488 (1.35), 7.948 (4.58), 7.951 (5.93), 7.968 (5.30), 7.972 (4.22), 8.022 (16.00), 9.114 (0.72).

### Example 25

### 8-bromo-N-[(4,5-difluoro-1H-benzimidazol-2-yl)methyl]-2-(morpholin-4-yl)pyrazolo[1,5-a][1,3,5]triazin-4-amine

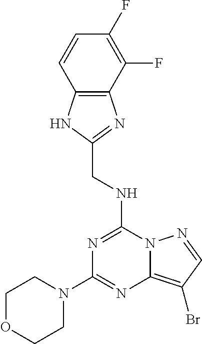 [SMILES: Fc1ccc2[nH]c(CNc3nc(N4CCOCC4)nc4c(Br)cnn34)nc2c1F]

8-bromo-N-[(4,5-difluoro-1H-benzimidazol-2-yl)methyl]-2-(methanesulfonyl)pyrazolo[1,5-a][1,3,5]triazin-4-amine (Intermediate 26, 65.0 mg, 90% purity, 128 μmol) and morpholine (110 μL, 1.3 mmol; CAS 110-91-8) were dissolved in acetonitrile (1.2 mL), N,N-diisopropylethylamine (67 μL, 380 μmol) was added and the mixture was stirred overnight at 70° C. The mixture was poured into water and extracted 3 times with a mixture of dichloromethane and 2-propanol. The organic phase was dried, evaporated and purified by preparative HPLC (HT basic) to give 21.0 mg (95% purity, 34% yield) of the title compound.

LC-MS (Method 2): Rₜ=1.00 min; MS (ESIpos): m/z=465 [M+H]⁺

¹H-NMR (400 MHz, DMSO-d6) δ[ppm]: 1.900 (1.02), 2.336 (0.97), 2.518 (10.79), 2.523 (7.84), 2.540 (0.91), 2.679 (0.91), 3.492 (2.85), 3.613 (3.97), 4.874 (7.84), 7.154 (0.59), 7.176 (1.23), 7.183 (0.70), 7.194 (1.29), 7.205 (1.34), 7.222 (1.56), 7.998 (0.43), 8.043 (16.00).

### Example 26

### 8-bromo-N²-ethyl-N⁴-{[4-(4-methylphenyl)-1H-imidazol-2-yl]methyl}pyrazolo[1,5-a][1,3,5]triazine-2,4-diamine

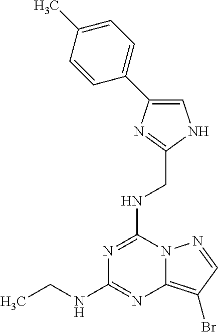 [SMILES: CCNc1nc(NCc2nc(-c3ccc(C)cc3)c[nH]2)n2ncc(Br)c2n1]

8-bromo-2-(methanesulfonyl)-N-{[4-(4-methylphenyl)-1H-imidazol-2-yl]methyl}pyrazolo[1,5-a][1,3,5]triazin-4-amine (Intermediate 28, 95.0 mg, 205 μmol), ethanamine (5.0 mL, 2.0 M in THF, 620 μmol) and N,N-diisopropylethylamine (530 μL, 3.0 mmol) were stirred for 2 h at 120° C. in the microwave. The mixture was evaporated, diluted with sat. sodium bicarbonate solution and dichloromethane. The layers were separated and the aqueous phase was extracted 3 times with a mixture of dichloromethane and 2-propanol (4:1). The combined organic layers were dried and evaporated. The residue was purified by preparative HPLC (HT basic), followed by flash chromatography (dichloromethane/ethanol gradient 0-6% ethanol), to give 4.50 mg (90% purity, 5% yield) of the title compound.

LC-MS (Method 1): Rₜ=0.94 min; MS (ESIpos): m/z=427 [M+H]⁺

¹H-NMR (400 MHz, METHANOL-d4) 5 [ppm]: 0.100 (0.43), 0.900 (0.45), 1.178 (1.16), 1.287 (1.93), 1.306 (0.84), 1.339 (0.52), 2.329 (16.00), 4.815 (2.73), 7.160 (4.02), 7.180 (4.46), 7.262 (4.19), 7.537 (5.06), 7.557 (4.52), 7.746 (4.45).

### Example 27

### 8-bromo-N-[(5-methyl-4-phenyl-1H-imidazol-2-yl)methyl]-2-(morpholin-4-yl)pyrazolo[1,5-a][1,3,5]triazin-4-amine

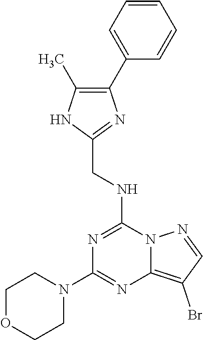 [SMILES: Cc1[nH]c(CNc2nc(N3CCOCC3)nc3c(Br)cnn23)nc1-c1ccccc1]

{[8-bromo-2-(morpholin-4-yl)pyrazolo[1,5-a][1,3,5]triazin-4-yl]amino}acetaldehyde (Intermediate 32, 150 mg, 440 μmol) and 1-phenylpropane-1,2-dione (59 μL, 440 μmol) were dissolved in tetrahydrofurane (10 mL), ammonium acetate (339 mg, 4.40 mmol) dissolved in methanol (5.0 mL) was added and the mixture was stirred for 4 h at rt. The mixture was evaporated and purified by flash chromatography (hexane/ethyl acetate gradient 0-100% ethyl acetate), to give 56.8 mg (97% purity, 27% yield) of the title compound.

LC-MS (Method 1): Rₜ=0.90 min; MS (ESIpos): m/z=469 [M+H]⁺

¹H-NMR (400 MHz, DMSO-d6) δ[ppm]: 1.027 (0.26), 1.042 (0.27), 1.230 (0.27), 1.259 (0.21), 2.242 (0.99), 2.322 (0.32), 2.327 (0.48), 2.332 (0.67), 2.343 (5.35), 2.518 (1.14), 2.522 (0.71), 2.664 (0.20), 2.669 (0.29), 2.673 (0.22), 3.160 (2.59), 3.171 (2.67), 3.332 (16.00), 3.692 (2.45), 3.703 (2.74), 4.097 (0.50), 4.111 (0.48), 4.663 (1.80), 7.145 (0.32), 7.164 (0.73), 7.183 (0.47), 7.324 (0.89), 7.344 (1.55), 7.363 (0.84), 7.399 (0.16), 7.418 (0.36), 7.435 (0.73), 7.591 (1.48), 7.610 (1.24), 8.005 (4.20), 8.943 (0.39), 11.714 (0.54).

### Example 28

### N-[(7-methyl-3H-imidazo[4,5-b]pyridin-2-yl)methyl]-2-(morpholin-4-yl)-8-(propan-2-yl)pyrazolo[1,5-a][1,3,5]triazin-4-amine

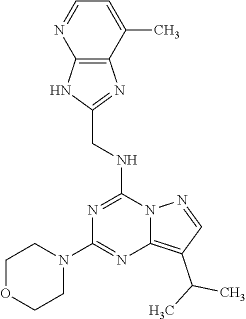 [SMILES: Cc1ccnc2[nH]c(CNc3nc(N4CCOCC4)nc4c(C(C)C)cnn34)nc12]

2-(methanesulfonyl)-N-[(7-methyl-3H-imidazo[4,5-b]pyridin-2-yl)methyl]-8-(propan-2-yl)pyrazolo[1,5-a][1,3,5]triazin-4-amine (Intermediate 34, 95.0 mg, 237 μmol) and morpholine (100 μL, 1.2 mmol; CAS 110-91-8) were dissolved in acetonitrile (2.2 mL), N,N-diisopropylethylamine (120 μL, 710 μmol) was added and the mixture was stirred overnight at 70° C. The mixture was purified by preparative HPLC (HT basic), followed by precipitation from methanol to give 4.0 mg (95% purity, 4% yield) of the title compound.

LC-MS (Method 2): Rₜ=1.04 min; MS (ESIpos): m/z=408 [M+H]⁺

¹H-NMR (400 MHz, DMSO-d6) δ[ppm]: 1.234 (15.83), 1.251 (16.00), 2.331 (2.38), 2.336 (1.16), 2.518 (15.11), 2.522 (9.41), 2.673 (2.38), 2.678 (1.11), 2.926 (1.00), 2.943 (1.27), 2.960 (0.94), 3.469 (2.88), 3.546 (3.10), 3.558 (3.49), 4.847 (2.99), 6.980 (1.49), 6.992 (1.49), 7.799 (5.54), 8.101 (0.94), 8.111 (0.89).

### Example 29

### 8-bromo-N-{[5-(4-fluorophenyl)-4H-1,2,4-triazol-3-yl]methyl}-2-(morpholin-4-yl)pyrazolo[1,5-a][1,3,5]triazin-4-amine

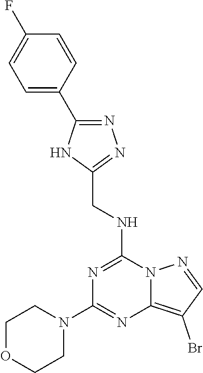 [SMILES: Fc1ccc(-c2nnc(CNc3nc(N4CCOCC4)nc4c(Br)cnn34)[nH]2)cc1]

2-{[8-bromo-2-(morpholin-4-yl)pyrazolo[1,5-a][1,3,5]triazin-4-yl]amino}acetohydrazide (Intermediate 39, 100 mg, 269 μmol) and 4-fluorobenzene-1-carboximidamide hydrochloride (68.8 mg, 394 μmol) were dissolved in DMF (2.0 mL) and stirred for 45 min at 150° C. in the microwave. The mixture was purified by preparative HPLC twice (1. HT basic, 2. see below) to give 3.80 mg (97% purity, 3% yield) of the title compound.

### Preparative HPLC Method

Instrument: Waters Autopurificationsystem; Column: Reprospher C18 10μ, 125×30 mm; eluent A: water+0.1 vol % formic acid; eluent B: acetonitrile; gradient: 0.0-0.5 min 20% B (35-70 mL/min), 0.5-8.5 min 20-90% B; flow: 70 mL/min; temperature: 25° C.; DAD scan: 210-400 nm.

### Analytical HPLC Method

Instrument: Waters Acquity UPLCMS SingleQuad; Column: Acquity UPLC BEH C18 1.7μ, 50×2.1 mm; eluent A: water+0.1 vol % formic acid; eluent B: acetonitrile; gradient: 0-2.6 min 1-99% B, 2.6-3.0 min 99% B; flow: 0.8 mL/min; temperature: 60° C.; DAD scan: 210-400 nm.

Analytical HPLC: Rₜ=1.47 min; MS (ESIpos): m/z=474 [M+H]⁺

¹H-NMR (400 MHz, DMSO-d6) δ[ppm]: 1.231 (0.98), 2.331 (1.31), 2.336 (0.58), 2.518 (7.60), 2.523 (4.70), 2.539 (0.49), 2.673 (1.31), 2.678 (0.58), 3.545 (5.47), 3.647 (5.77), 3.659 (6.81), 4.766 (3.27), 7.282 (1.68), 7.303 (3.21), 7.325 (1.74), 7.969 (0.58), 7.976 (4.55), 7.982 (1.92), 7.990 (5.16), 7.999 (5.16), 8.007 (1.86), 8.013 (4.55), 8.021 (16.00), 9.124 (0.92).

### Example 30

### 2-(morpholin-4-yl)-N-[(5-phenyl-1H-imidazol-2-yl)methyl]-8-(propan-2-yl)pyrazolo[1,5-a][1,3,5]triazin-4-amine

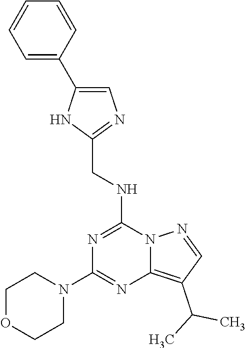 [SMILES: CC(C)c1cnn2c(NCc3ncc(-c4ccccc4)[nH]3)nc(N3CCOCC3)nc12]

2-(methanesulfonyl)-N-[(5-phenyl-1H-imidazol-2-yl)methyl]-8-(propan-2-yl)pyrazolo[1,5-a][1,3,5]triazin-4-amine (Intermediate 41, 95.0 mg, 231 μmol) and morpholine (100 μL, 1.2 mmol; CAS-RN:[110-91-8]) were dissolved in acetonitrile (2.2 mL), N,N-diisopropylethylamine (120 μL, 690 μmol) was added and the mixture was stirred overnight at 70° C. The mixture was purified by preparative HPLC (HT basic) to give 36.0 mg (97% purity, 36% yield) of the title compound.

LC-MS (Method 3): Rₜ=1.18 min; MS (ESIpos): m/z=419 [M+H]⁺

¹H-NMR (400 MHz, DMSO-d6) δ[ppm]: 1.230 (15.80), 1.247 (16.00), 2.322 (0.51), 2.326 (0.70), 2.331 (0.51), 2.522 (2.24), 2.539 (1.06), 2.664 (0.53), 2.668 (0.69), 2.673 (0.51), 2.905 (0.41), 2.922 (1.04), 2.940 (1.36), 2.957 (0.96), 3.564 (3.49), 3.633 (3.56), 3.644 (3.86), 4.688 (2.51), 7.151 (0.78), 7.168 (0.57), 7.294 (0.93), 7.312 (1.62), 7.331 (0.91), 7.500 (1.55), 7.718 (1.55), 7.737 (1.39), 7.776 (6.30), 8.711 (0.49), 11.916 (0.47).

### Example 31

### 2-(4-methylpiperazin-1-yl)-N-[(5-phenyl-1H-imidazol-2-yl)methyl]-8-(propan-2-yl)pyrazolo[1,5-a][1,3,5]triazin-4-amine

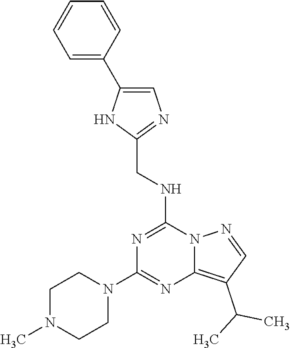 [SMILES: CC(C)c1cnn2c(NCc3ncc(-c4ccccc4)[nH]3)nc(N3CCN(C)CC3)nc12]

2-(methanesulfonyl)-N-[(5-phenyl-1H-imidazol-2-yl)methyl]-8-(propan-2-yl)pyrazolo[1,5-a][1,3,5]triazin-4-amine (Intermediate 41, 95.0 mg, 231 μmol) and 1-methylpiperazine (92.5 mg, 923 μmol) were dissolved in acetonitrile (2.2 mL), N,N-diisopropylethylamine (120 μL, 690 μmol) was added and the mixture was stirred overnight at 70° C. The mixture was purified by preparative HPLC (HT basic) to give 38.5 mg (99% purity, 38% yield) of the title compound.

LC-MS (Method 3): Rₜ=1.16 min; MS (ESIpos): m/z=432 [M+H]⁺

¹H-NMR (400 MHz, DMSO-d6) δ[ppm]: 1.227 (15.21), 1.244 (16.00), 2.152 (4.75), 2.266 (2.53), 2.331 (1.19), 2.336 (0.55), 2.518 (6.31), 2.523 (3.91), 2.673 (1.14), 2.678 (0.50), 2.917 (0.98), 2.934 (1.29), 2.951 (0.92), 3.662 (2.80), 4.678 (1.93), 4.687 (1.95), 7.150 (0.74), 7.168 (0.53), 7.292 (0.90), 7.311 (1.53), 7.330 (0.84), 7.497 (1.53), 7.718 (1.48), 7.737 (1.32), 7.757 (6.52), 8.649 (0.48), 11.918 (0.50).

### Example 32

### N-[(7-methyl-3H-imidazo[4,5-b]pyridin-2-yl)methyl]-2-(4-methylpiperazin-1-yl)-8-(propan-2-yl)pyrazolo[1,5-a][1,3,5]triazin-4-amine

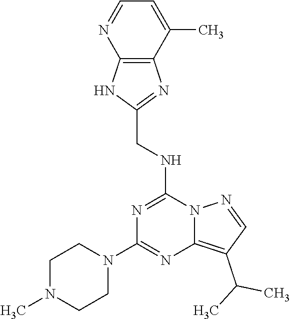 [SMILES: Cc1ccnc2[nH]c(CNc3nc(N4CCN(C)CC4)nc4c(C(C)C)cnn34)nc12]

2-(methanesulfonyl)-N-[(7-methyl-3H-imidazo[4,5-b]pyridin-2-yl)methyl]-8-(propan-2-yl)pyrazolo[1,5-a][1,3,5]triazin-4-amine (Intermediate 34, 120 mg, 300 μmol) and 1-methylpiperazine (150 mg, 1.50 mmol) were dissolved in acetonitrile (2.8 mL), N,N-diisopropylethylamine (160 μL, 900 μmol) was added and the mixture was stirred overnight at 70° C. The mixture was purified by preparative HPLC (HT basic), followed by precipitation from methanol give 3.5 mg (99% purity, 3% yield) of the title compound.

LC-MS (Method 2): Rₜ=0.96 min; MS (ESIpos): m/z=421 [M+H]⁺

¹H-NMR (400 MHz, DMSO-d6) δ[ppm]: 1.229 (16.00), 1.247 (15.97), 2.094 (10.38), 2.129 (2.13), 2.331 (1.06), 2.336 (0.56), 2.518 (6.35), 2.522 (3.95), 2.673 (1.06), 2.678 (0.48), 2.917 (0.99), 2.935 (1.32), 2.952 (0.94), 3.561 (2.71), 4.837 (2.99), 6.980 (1.72), 6.982 (1.72), 6.994 (1.80), 7.780 (5.85), 8.101 (1.16), 8.113 (1.11), 8.807 (0.43).

### Example 33

### 2-(morpholin-4-yl)-N-[(5-phenyl-4H-1,2,4-triazol-3-yl)methyl]-8-(propan-2-yl)pyrazolo[1,5-a][1,3,5]triazin-4-amine

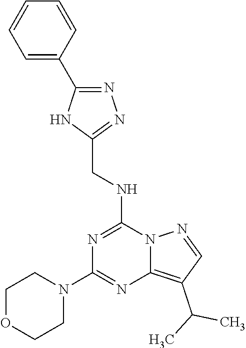 [SMILES: CC(C)c1cnn2c(NCc3nnc(-c4ccccc4)[nH]3)nc(N3CCOCC3)nc12]

2-(methanesulfonyl)-N-[(5-phenyl-4H-1,2,4-triazol-3-yl)methyl]-8-(propan-2-yl)pyrazolo[1,5-a][1,3,5]triazin-4-amine (Intermediate 43, 120 mg, 291 μmol) and morpholine (130 μL, 1.5 mmol; CAS 110-91-8) were dissolved in acetonitrile (2.8 mL), N,N-diisopropylethylamine (150 μL, 870 μmol) was added and the mixture was stirred overnight at 70° C. The mixture was purified by preparative HPLC (HT basic) to give 39.5 mg (99% purity, 32% yield) of the title compound.

LC-MS (Method 3): Rₜ=0.95 min; MS (ESIpos): m/z=420 [M+H]⁺

¹H-NMR (400 MHz, DMSO-d6) δ[ppm]: 1.229 (15.98), 1.246 (16.00), 2.331 (0.49), 2.518 (2.84), 2.522 (1.71), 2.673 (0.50), 2.921 (0.94), 2.938 (1.24), 2.955 (0.89), 3.538 (3.18), 3.549 (2.78), 3.604 (2.94), 3.615 (3.34), 3.625 (1.75), 4.746 (1.68), 7.414 (0.93), 7.421 (0.41), 7.431 (1.10), 7.446 (1.83), 7.465 (2.20), 7.482 (0.82), 7.486 (0.56), 7.787 (5.71), 7.946 (2.34), 7.949 (2.93), 7.954 (1.37), 7.961 (0.82), 7.966 (2.81), 7.970 (2.08), 8.829 (0.45).

### Example 34

### N²-ethyl-N⁴-[(5-phenyl-1H-imidazol-2-yl)methyl]-8-(propan-2-yl)pyrazolo[1,5-a][1,3,5]triazine-2,4-diamine

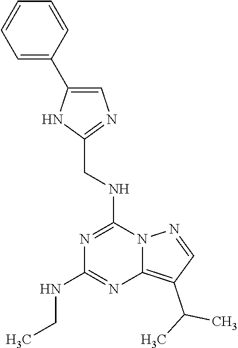 [SMILES: CCNc1nc(NCc2ncc(-c3ccccc3)[nH]2)n2ncc(C(C)C)c2n1]

2-(methanesulfonyl)-N-[(5-phenyl-1H-imidazol-2-yl)methyl]-8-(propan-2-yl)pyrazolo[1,5-a][1,3,5]triazin-4-amine (Intermediate 41, 100 mg, 243 μmol) and ethanamine hydrochloride (99.1 mg, 1.22 mmol) were dissolved in acetonitrile (2.3 mL), N,N-diisopropylethylamine (210 μL, 1.2 mmol) was added and the mixture was stirred overnight at 70° C. The mixture was purified by preparative HPLC (HT basic) to give 40.5 mg (98% purity, 43% yield) of the title compound.

LC-MS (Method 3): Rₜ=1.18 min; MS (ESIpos): m/z=377 [M+H]⁺

¹H-NMR (400 MHz, DMSO-d6) δ[ppm]: 0.932 (0.94), 0.948 (0.94), 1.094 (1.78), 1.111 (2.35), 1.164 (0.46), 1.231 (16.00), 1.248 (15.86), 2.331 (1.03), 2.336 (0.46), 2.518 (6.21), 2.522 (3.79), 2.539 (1.70), 2.673 (1.03), 2.678 (0.48), 2.894 (0.46), 2.911 (1.08), 2.928 (1.39), 2.945 (1.01), 2.963 (0.41), 3.231 (0.67), 3.249 (1.99), 3.265 (2.61), 3.281 (1.99), 3.299 (0.74), 4.673 (2.61), 6.942 (1.13), 6.956 (1.92), 6.969 (0.96), 7.134 (0.72), 7.152 (1.68), 7.170 (1.15), 7.294 (2.01), 7.314 (3.53), 7.333 (1.92), 7.509 (3.43), 7.706 (11.08), 7.725 (3.41), 7.744 (3.02), 8.497 (0.62), 11.862 (0.91).

### Example 35

### 2-methyl-1-{methyl[4-{[(5-phenyl-1H-imidazol-2-yl)methyl]amino}-8-(propan-2-yl)pyrazolo[1,5-a][1,3,5]triazin-2-yl]amino}propan-2-ol

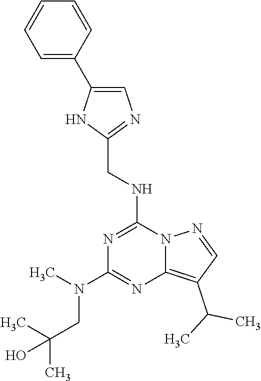 [SMILES: CC(C)c1cnn2c(NCc3ncc(-c4ccccc4)[nH]3)nc(N(C)CC(C)(C)O)nc12]

2-(methanesulfonyl)-N-[(5-phenyl-1H-imidazol-2-yl)methyl]-8-(propan-2-yl)pyrazolo[1,5-a][1,3,5]triazin-4-amine (Intermediate 41, 100 mg, 243 μmol) and 2-methyl-1-(methylamino)propan-2-ol hydrochloride (170 mg, 1.22 mmol) were dissolved in acetonitrile (2.3 mL), N,N-diisopropylethylamine (210 μL, 1.2 mmol) was added and the mixture was stirred overnight at 70° C. The mixture was purified by preparative HPLC (HT basic) to give 31.5 mg (95% purity, 28% yield) of the title compound.

LC-MS (Method 3): Rₜ=1.20 min; MS (ESIpos): m/z=435 [M+H]⁺

¹H-NMR (400 MHz, DMSO-d6) δ[ppm]: 0.970 (3.27), 1.014 (1.22), 1.066 (1.14), 1.104 (2.17), 1.236 (10.26), 1.252 (10.15), 2.331 (1.63), 2.336 (0.76), 2.518 (9.39), 2.523 (5.89), 2.539 (3.46), 2.673 (1.63), 2.678 (0.76), 2.926 (0.61), 3.140 (16.00), 3.544 (3.23), 4.401 (0.46), 4.658 (1.37), 7.127 (0.65), 7.145 (1.48), 7.163 (1.06), 7.287 (1.71), 7.306 (3.04), 7.325 (1.71), 7.471 (0.87), 7.718 (3.08), 7.731 (13.91), 8.557 (0.42), 11.912 (0.84).

### Example 36

### N-[(5-phenyl-1H-imidazol-2-yl)methyl]-2-(piperidin-1-yl)-8-(propan-2-yl)pyrazolo[1,5-a][1,3,5]triazin-4-amine

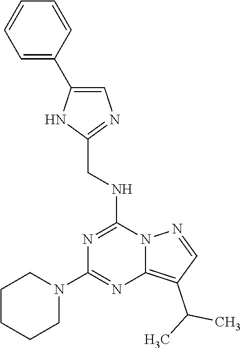 [SMILES: CC(C)c1cnn2c(NCc3ncc(-c4ccccc4)[nH]3)nc(N3CCCCC3)nc12]

2-(methanesulfonyl)-N-[(5-phenyl-1H-imidazol-2-yl)methyl]-8-(propan-2-yl)pyrazolo[1,5-a][1,3,5]triazin-4-amine (Intermediate 41, 100 mg, 243 μmol) and piperidine (120 μL, 1.2 mmol) were dissolved in acetonitrile (2.3 mL), N,N-diisopropylethylamine (130 μL, 730 μmol) was added and the mixture was stirred overnight at 70° C. The mixture was purified by preparative HPLC (HT basic) to give 33.0 mg (98% purity, 32% yield) of the title compound.

LC-MS (Method 3): Rₜ=1.40 min; MS (ESIpos): m/z=417 [M+H]⁺

¹H-NMR (400 MHz, DMSO-d6) δ[ppm]: 1.228 (15.70), 1.246 (16.00), 1.434 (1.70), 1.543 (0.90), 1.555 (0.90), 2.518 (3.84), 2.522 (2.46), 2.539 (0.44), 2.673 (0.69), 2.911 (0.92), 2.928 (1.22), 2.945 (0.87), 3.655 (2.04), 3.668 (2.73), 3.681 (2.01), 4.675 (1.86), 7.150 (0.50), 7.294 (0.58), 7.312 (1.00), 7.329 (0.62), 7.492 (0.70), 7.717 (0.89), 7.734 (6.75).

### Example 37

### 2-(4-methylpiperazin-1-yl)-N-[(5-phenyl-4H-1,2,4-triazol-3-yl)methyl]-8-(propan-2-yl)pyrazolo[1,5-a][1,3,5]triazin-4-amine

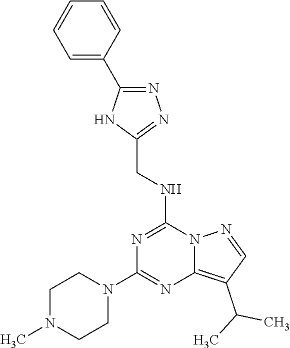 [SMILES: CC(C)c1cnn2c(NCc3nnc(-c4ccccc4)[nH]3)nc(N3CCN(C)CC3)nc12]

2-(methanesulfonyl)-N-[(5-phenyl-4H-1,2,4-triazol-3-yl)methyl]-8-(propan-2-yl)pyrazolo[1,5-a][1,3,5]triazin-4-amine (Intermediate 43, 100 mg, 242 μmol) and 1-methylpiperazine (140 μL, 1.2 mmol) were dissolved in acetonitrile (2.3 mL), N,N-diisopropylethylamine (130 μL, 730 μmol) was added and the mixture was stirred overnight at 70° C. The mixture was purified by preparative HPLC (HT basic) to give 34.4 mg (99% purity, 32% yield) of the title compound.

LC-MS (Method 3): Rₜ=0.94 min; MS (ESIpos): m/z=433 [M+H]⁺

¹H-NMR (400 MHz, DMSO-d6) δ[ppm]: 1.226 (15.46), 1.243 (16.00), 2.125 (9.65), 2.223 (2.82), 2.331 (0.97), 2.336 (0.45), 2.518 (5.13), 2.522 (3.19), 2.539 (0.67), 2.673 (0.95), 2.678 (0.43), 2.914 (0.97), 2.931 (1.27), 2.948 (0.90), 3.627 (2.69), 4.735 (1.66), 7.412 (0.95), 7.429 (1.12), 7.444 (1.90), 7.462 (2.24), 7.479 (0.84), 7.768 (5.92), 7.945 (2.37), 7.949 (3.01), 7.953 (1.42), 7.961 (0.82), 7.966 (2.82), 7.970 (2.11), 8.772 (0.45).

### Example 38

### N²-ethyl-N⁴-[(5-phenyl-4H-1,2,4-triazol-3-yl)methyl]-8-(propan-2-yl)pyrazolo[1,5-a][1,3,5]triazine-2,4-diamine

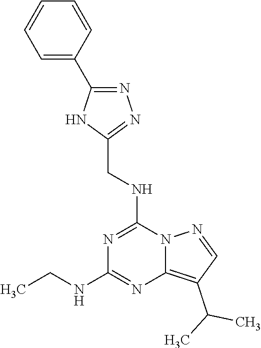 [SMILES: CCNc1nc(NCc2nnc(-c3ccccc3)[nH]2)n2ncc(C(C)C)c2n1]

2-(methanesulfonyl)-N-[(5-phenyl-4H-1,2,4-triazol-3-yl)methyl]-8-(propan-2-yl)pyrazolo[1,5-a][1,3,5]triazin-4-amine (Intermediate 43, 100 mg, 242 μmol) and ethanamine hydrochloride (98.8 mg, 1.21 mmol) were dissolved in acetonitrile (2.3 mL), N,N-diisopropylethylamine (130 μL, 730 μmol) was added and the mixture was stirred overnight at 70° C. The mixture was purified by preparative HPLC (HT basic) to give 21.7 mg (95% purity, 23% yield) of the title compound.

LC-MS (Method 3): Rₜ=0.91 min; MS (ESIpos): m/z=378 [M+H]⁺

¹H-NMR (400 MHz, DMSO-d6) δ[ppm]: 0.918 (2.15), 0.932 (15.59), 0.935 (5.50), 0.949 (15.75), 0.953 (2.98), 1.093 (1.94), 1.234 (15.96), 1.251 (16.00), 2.331 (1.78), 2.336 (0.83), 2.388 (0.45), 2.406 (1.28), 2.424 (1.28), 2.442 (0.50), 2.518 (10.05), 2.522 (6.28), 2.539 (1.78), 2.673 (1.78), 2.678 (0.79), 2.913 (1.12), 2.931 (1.49), 2.943 (1.53), 2.959 (1.41), 2.975 (0.87), 3.239 (1.90), 4.740 (5.09), 4.753 (5.13), 6.896 (1.28), 6.910 (2.36), 6.923 (1.28), 7.396 (0.66), 7.414 (2.23), 7.431 (2.81), 7.445 (4.75), 7.464 (5.58), 7.481 (2.11), 7.720 (14.59), 7.954 (5.99), 7.957 (7.69), 7.973 (7.03), 7.978 (5.46), 8.562 (0.41).

### Example 39

### 2-methyl-1-{methyl[4-{[(5-phenyl-4H-1,2,4-triazol-3-yl)methyl]amino}-8-(propan-2-yl)pyrazolo[1,5-a][1,3,5]triazin-2-yl]amino}propan-2-ol

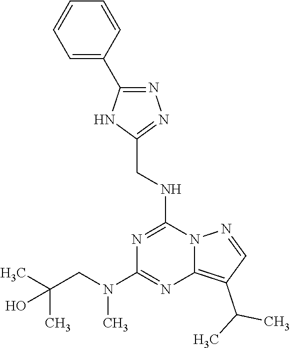 [SMILES: CC(C)c1cnn2c(NCc3nnc(-c4ccccc4)[nH]3)nc(N(C)CC(C)(C)O)nc12]

2-(methanesulfonyl)-N-[(5-phenyl-4H-1,2,4-triazol-3-yl)methyl]-8-(propan-2-yl)pyrazolo[1,5-a][1,3,5]triazin-4-amine (Intermediate 43, 100 mg, 242 μmol) and 2-methyl-1-(methylamino)propan-2-ol hydrochloride (169 mg, 1.21 mmol) were dissolved in acetonitrile (2.3 mL), N,N-diisopropylethylamine (130 μL, 730 μmol) was added and the mixture was stirred overnight at 70° C. The mixture was purified by preparative HPLC (HT basic) to give 14.4 mg (95% purity, 13% yield) of the title compound.

LC-MS (Method 3): Rₜ=0.96 min; MS (ESIpos): m/z=436 [M+H]⁺

¹H-NMR (400 MHz, DMSO-d6) δ[ppm]: 0.871 (5.46), 0.917 (0.90), 0.932 (2.25), 0.948 (2.12), 0.952 (0.64), 1.066 (1.80), 1.089 (2.25), 1.101 (2.96), 1.236 (12.98), 1.253 (12.47), 1.282 (0.77), 2.332 (2.76), 2.336 (1.16), 2.518 (14.78), 2.522 (9.38), 2.539 (13.88), 2.673 (2.70), 2.678 (1.16), 2.941 (0.77), 3.105 (12.40), 3.467 (1.93), 3.534 (0.84), 3.578 (0.51), 4.700 (1.61), 4.766 (0.84), 7.388 (0.71), 7.407 (2.63), 7.425 (3.08), 7.438 (5.20), 7.457 (6.17), 7.474 (2.31), 7.680 (0.45), 7.742 (16.00), 7.945 (4.95), 7.963 (4.50), 8.687 (0.64).

### Example 40

### 8-bromo-N-{[5-(3-methylphenyl)-4H-1,2,4-triazol-3-yl]methyl}-2-(morpholin-4-yl)pyrazolo[1,5-a][1,3,5]triazin-4-amine

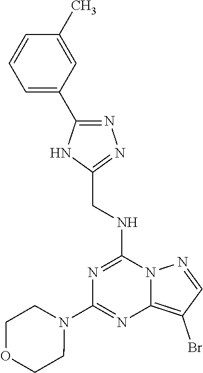 [SMILES: Cc1cccc(-c2nnc(CNc3nc(N4CCOCC4)nc4c(Br)cnn34)[nH]2)c1]

2-{[8-bromo-2-(morpholin-4-yl)pyrazolo[1,5-a][1,3,5]triazin-4-yl]amino}acetohydrazide (Intermediate 39, 105 mg, 283 μmol) and 3-methylbenzene-1-carboximidamide hydrochloride (70.0 mg, 410 μmol) were dissolved in DMF (2.0 mL) and stirred for 45 min at 150° C. in the microwave. The mixture was purified by preparative HPLC to give 10.7 mg (95% purity, 8% yield) of the title compound.

### Preparative HPLC Method

Instrument: Waters Autopurificationsystem; Column: XBrigde C18 5μ, 100×30 mm; eluent A: water+0.1 vol % formic acid; eluent B: acetonitrile; gradient: 0.0-0.5 min 30% B (35-70 mL/min), 0.5-8.5 min 30-45% B; flow: 70 mL/min; temperature: 25° C.; DAD scan: 210-400 nm.

### Analytical HPLC Method

Instrument: Waters Acquity UPLCMS SingleQuad; Column: Acquity UPLC BEH C18 1.7μ, 50×2.1 mm; eluent A: water+0.1 vol % formic acid; eluent B: acetonitrile; gradient: 0-2.6 min 1-99% B, 2.6-3.0 min 99% B; flow: 0.8 mL/min; temperature: 60° C.; DAD scan: 210-400 nm.

Analytical HPLC: Rₜ=1.52 min; MS (ESIpos): m/z=470 [M+H]⁺

¹H-NMR (400 MHz, DMSO-d6) δ[ppm]: 1.231 (1.06), 2.331 (1.45), 2.336 (0.74), 2.357 (16.00), 2.518 (7.68), 2.523 (4.74), 2.539 (0.90), 2.673 (1.39), 2.678 (0.61), 3.548 (4.39), 3.654 (4.45), 3.665 (5.29), 4.757 (2.19), 7.231 (0.87), 7.248 (1.13), 7.332 (0.87), 7.351 (1.39), 7.370 (0.68), 7.740 (1.94), 7.759 (1.77), 7.790 (3.35), 8.021 (13.16), 9.102 (0.52).

### Example 41

### 8-bromo-N-{[5-(2,4-difluorophenyl)-4H-1,2,4-triazol-3-yl]methyl}-2-(morpholin-4-yl)pyrazolo[1,5-a][1,3,5]triazin-4-amine

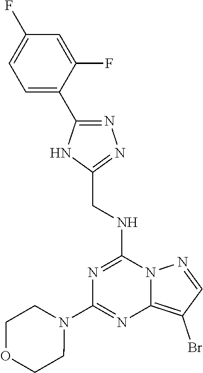 [SMILES: Fc1ccc(-c2nnc(CNc3nc(N4CCOCC4)nc4c(Br)cnn34)[nH]2)c(F)c1]

2-{[8-bromo-2-(morpholin-4-yl)pyrazolo[1,5-a][1,3,5]triazin-4-yl]amino}acetohydrazide (Intermediate 39, 105 mg, 283 μmol) and 2,4-difluorobenzene-1-carboximidamide hydrochloride (79.0 mg, 410 μmol) were dissolved in DMF (2.0 mL) and stirred for 45 min at 150° C. in the microwave. The mixture was purified by preparative HPLC to give 9.60 mg (95% purity, 7% yield) of the title compound.

### Preparative HPLC Method

Instrument: Waters Autopurificationsystem; Column: XBrigde C18 5μ, 100×30 mm; eluent A: water+0.1 vol % formic acid; eluent B: acetonitrile; gradient: 0.0-0.5 min 30% B (35-70 mL/min), 0.5-8.5 min 30-45% B; flow: 70 mL/min; temperature: 25° C.; DAD scan: 210-400 nm.

### Analytical HPLC Method

Instrument: Waters Acquity UPLCMS SingleQuad; Column: Acquity UPLC BEH C18 1.7μ, 50×2.1 mm; eluent A: water+0.1 vol % formic acid; eluent B: acetonitrile; gradient: 0-2.6 min 1-99% B, 2.6-3.0 min 99% B; flow: 0.8 mL/min; temperature: 60° C.; DAD scan: 210-400 nm.

Analytical HPLC: Rₜ=1.47 min; MS (ESIpos): m/z=479 [M+H]⁺

¹H-NMR (400 MHz, DMSO-d6) δ[ppm]: 1.231 (1.22), 2.074 (2.27), 2.331 (1.77), 2.336 (0.80), 2.518 (9.98), 2.522 (6.32), 2.669 (2.44), 2.673 (1.77), 2.678 (0.80), 3.550 (5.22), 3.645 (5.60), 3.656 (6.53), 4.781 (4.55), 7.184 (0.76), 7.190 (0.80), 7.210 (1.52), 7.226 (0.80), 7.232 (0.84), 7.381 (0.63), 7.405 (1.18), 7.428 (0.63), 7.957 (1.26), 7.974 (1.47), 7.980 (2.40), 7.996 (2.44), 8.001 (1.39), 8.021 (16.00), 9.133 (0.72).

### Example 42

### 3-[2-({[8-bromo-2-(morpholin-4-yl)pyrazolo[1,5-a][1,3,5]triazin-4-yl]amino}methyl)-1H-imidazol-4-yl]benzonitrile

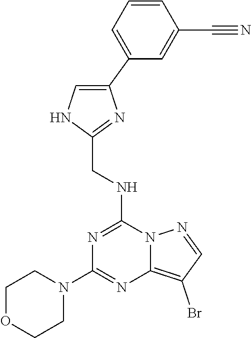 [SMILES: N#Cc1cccc(-c2c[nH]c(CNc3nc(N4CCOCC4)nc4c(Br)cnn34)n2)c1]

{[8-bromo-2-(morpholin-4-yl)pyrazolo[1,5-a][1,3,5]triazin-4-yl]amino}ethanimidamide (Intermediate 47, 205 mg, 577 μmol) and 3-(bromoacetyl)benzonitrile (168 mg, 750 μmol) were dissolved in acetonitrile (10 mL), potassium carbonate (319 mg, 2.3 mmol) was added and the mixture was stirred for 16 h at reflux. The mixture was evaporated, diluted with dichloromethane and water and the layers were separated. The aqueous layer was extracted 3 times with a mixture of dichloromethane and 2-propanol (4:1) and the combined organic layers were dried, evaporated and purified by preparative HPLC (HT basic), followed by flash chromatography (dichloromethane/ethanol gradient 0-5% ethanol) to give 10.3 mg (90% purity, 3% yield) of the title compound.

LC-MS (Method 1): Rₜ=0.95 min; MS (ESIpos): m/z=480 [M+H]⁺

¹H-NMR (400 MHz, DMSO-d6) δ[ppm]: 1.027 (0.47), 1.042 (0.47), 1.232 (0.58), 1.988 (0.41), 2.518 (3.95), 2.523 (2.75), 3.159 (2.09), 3.172 (2.16), 3.566 (16.00), 3.670 (2.40), 3.682 (2.69), 3.693 (1.52), 4.095 (0.47), 4.108 (0.43), 4.706 (1.58), 4.719 (1.51), 7.517 (0.67), 7.537 (1.64), 7.557 (1.14), 7.594 (0.92), 7.597 (1.48), 7.601 (0.98), 7.613 (0.62), 7.617 (0.86), 7.620 (0.53), 7.731 (1.84), 7.735 (1.79), 8.012 (6.82), 8.054 (0.71), 8.057 (1.07), 8.062 (0.74), 8.074 (0.64), 8.078 (1.02), 8.081 (0.68), 8.131 (1.20), 8.135 (1.88), 8.138 (1.04), 9.026 (0.68), 12.107 (0.71).

### Example 43

### 4-{[(1H-benzimidazol-2-yl)methyl]amino}-2-(morpholin-4-yl)pyrazolo[1,5-a][1,3,5]triazine-8-carbonitrile

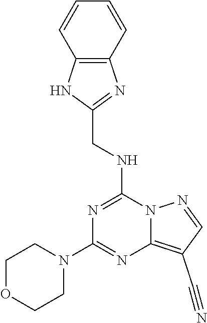 [SMILES: N#Cc1cnn2c(NCc3nc4ccccc4[nH]3)nc(N3CCOCC3)nc12]

N-[(1H-benzimidazol-2-yl)methyl]-8-bromo-2-(morpholin-4-yl)pyrazolo[1,5-a][1,3,5]triazin-4-amine (Example 4, 60.0 mg, 140 μmol), bis[cinnamyl palladium(II) chloride] (3.62 mg, 6.99 μmol; CAS 12131-44-1), zinc cyanide (16.4 mg, 140 μmol; CAS 557-21-1) and 1,1′-bis(diphenylphosphanyl)ferrocene (3.87 mg, 6.99 μmol; CAS 12150-46-8) were sealed in a vessel and flushed with argon. N,N-dimethylacetamide (1.0 mL, pre-flushed with argon) and N,N-diisopropylethylamine (49 μL, 280 μmol) were added. The mixture was stirred at 80° C. overnight. The mixture was diluted with ethyl acetate, filtered through a 2 g Silica column, washed with dichloromethane/MeOH (9:1) and the filtrate concentrated under reduced pressure. The residue was purified by preparative HPLC to give 7.90 mg (98% purity, 15% yield) of the title compound.

### Preparative HPLC Method

Instrument: Waters Autopurificationsystem; Column: XBrigde C18 5μ, 50×50 mm; eluent A: water+0.1 vol % formic acid; Eluent B: acetonitrile; gradient: 0.0-0.5 min 5% B (50-100 mL/min), 0.5-8.0 min 5-35% B; flow: 100 mL/min; temperature: 25° C.; DAD scan: 210-400 nm.

### Analytical HPLC Method

Instrument: Waters Acquity UPLCMS SingleQuad; Column: Acquity UPLC BEH C18 1.7μ, 50×2.1 mm; eluent A: water+0.1 vol % formic acid; eluent B: acetonitrile; gradient: 0-1.6 min 1-99% B, 1.6-2.0 min 99% B; flow: 0.8 mL/min; temperature: 60° C.; DAD scan: 210-400 nm.

Analytical HPLC: Rₜ=1.19 min; MS (ESIpos): m/z=376 [M+H]⁺

¹H-NMR (400 MHz, ACETONITRILE-d3) 5 [ppm]: 0.086 (1.88), 0.090 (2.00), 0.881 (0.51), 1.167 (16.00), 1.268 (2.59), 1.284 (1.20), 1.297 (0.77), 1.340 (0.50), 2.095 (0.64), 2.101 (0.92), 2.107 (1.19), 2.113 (1.35), 2.149 (8.35), 3.474 (0.48), 3.485 (0.96), 3.498 (1.09), 3.512 (0.49), 3.552 (15.09), 3.558 (5.23), 3.565 (5.53), 3.584 (0.99), 3.595 (0.71), 3.764 (1.26), 4.947 (1.99), 7.185 (1.49), 7.194 (1.33), 7.201 (1.44), 7.208 (1.61), 8.059 (3.90).

### Example 44

### 8-bromo-N-{[5-(3-fluorophenyl)-4H-1,2,4-triazol-3-yl]methyl}-2-(morpholin-4-yl)pyrazolo[1,5-a][1,3,5]triazin-4-amine

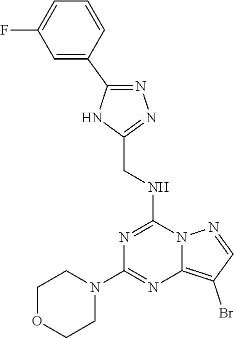 [SMILES: Fc1cccc(-c2nnc(CNc3nc(N4CCOCC4)nc4c(Br)cnn34)[nH]2)c1]

2-{[8-bromo-2-(morpholin-4-yl)pyrazolo[1,5-a][1,3,5]triazin-4-yl]amino}acetohydrazide (Intermediate 39, 80.0 mg, 216 μmol) and 3-fluorobenzene-1-carboximidamide hydrogen chloride (1/1) (45.2 mg, 259 μmol) were dissolved in DMF (2.4 mL), sodium ethanolate (29.3 mg, 431 μmol) was added and the mixture was stirred for 45 min at 180° C. in a microwave. The mixture was poured into water and extracted with ethyl acetate. The organic layer was dried, concentrated and purified by preparative HPLC (HT basic) to give 26.5 mg (24% yield) of the title compound.

LC-MS (Method 2): Rₜ=0.84 min; MS (ESIpos): m/z=474 [M+H]⁺

¹H-NMR (400 MHz, DMSO-d6) δ[ppm]: 1.899 (0.42), 2.337 (0.42), 2.518 (5.05), 2.523 (3.45), 3.544 (4.26), 3.644 (4.75), 3.656 (5.52), 3.667 (3.25), 4.784 (4.88), 7.245 (0.64), 7.261 (1.19), 7.267 (1.25), 7.283 (0.68), 7.289 (0.70), 7.491 (0.79), 7.511 (1.49), 7.527 (1.47), 7.531 (0.97), 7.546 (0.75), 7.674 (1.21), 7.677 (1.38), 7.680 (1.43), 7.684 (1.27), 7.699 (1.21), 7.703 (1.43), 7.705 (1.34), 7.709 (1.21), 7.799 (1.96), 7.802 (2.75), 7.805 (1.93), 7.818 (1.85), 7.822 (2.40), 7.824 (1.63), 8.000 (0.46), 8.026 (16.00).

### Example 45

### 8-bromo-N-{[5-(4-chlorophenyl)-4H-1,2,4-triazol-3-yl]methyl}-2-(morpholin-4-yl)pyrazolo[1,5-a][1,3,5]triazin-4-amine

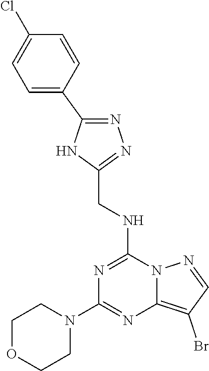 [SMILES: Clc1ccc(-c2nnc(CNc3nc(N4CCOCC4)nc4c(Br)cnn34)[nH]2)cc1]

2-{[8-bromo-2-(morpholin-4-yl)pyrazolo[1,5-a][1,3,5]triazin-4-yl]amino}acetohydrazide (Intermediate 39, 50.0 mg, 135 μmol) and 4-chlorobenzene-1-carboximidamide hydrogen iodide (1/1) (38.1 mg, 135 μmol) were provided in DMF (2.5 mL), sodium ethanolate (18.3 mg, 269 μmol) was added and the mixture was stirred in a microwave for 20 min at 100° C., for 1 h at 150° C. and for 20 min at 180° C. The mixture was poured into water and extracted with ethyl acetate. The organic layer was dried over sodium sulfate, filtered and concentrated. In a second preparation 2-{[8-bromo-2-(morpholin-4-yl)pyrazolo[1,5-a][1,3,5]triazin-4-yl]amino}acetohydrazide (Intermediate 39, 50.0 mg, 135 μmol) and 4-chlorobenzene-1-carboximidamide-hydrogen iodide (1/1) (38.1 mg, 135 μmol) were provided in DMSO (2.5 mL), sodium ethanolate (18.3 mg, 269 μmol) was added and the mixture was stirred in a microwave for 3 h at 150° C. The mixture was poured into water and extracted with ethyl acetate. The organic layer was dried over sodium sulfate, filtered and concentrated. The two crude products were purified together by preparative HPLC (HT basic) to give 5.7 mg (8% yield) of the title compound.

LC-MS (Method 2): Rₜ=0.89 min; MS (ESIpos): m/z=490 [M+H]⁺

¹H-NMR (400 MHz, DMSO-d6) δ[ppm]: 1.352 (0.18), 1.903 (0.51), 2.337 (0.66), 2.518 (8.62), 2.523 (5.91), 2.540 (1.91), 2.679 (0.66), 3.542 (5.58), 3.642 (6.06), 3.655 (7.08), 4.777 (4.33), 7.521 (4.04), 7.543 (4.37), 7.951 (1.43), 7.958 (10.57), 7.963 (3.12), 7.974 (3.08), 7.979 (9.28), 7.986 (1.10), 8.000 (0.37), 8.023 (16.00), 8.038 (0.62), 9.136 (0.70), 14.046 (0.22).

### Example 46

### N-[(1H-benzimidazol-2-yl)methyl]-8-bromo-2-(piperidin-1-yl)pyrazolo[1,5-a][1,3,5]triazin-4-amine

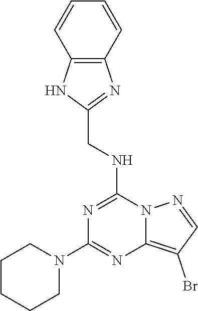 [SMILES: Brc1cnn2c(NCc3nc4ccccc4[nH]3)nc(N3CCCCC3)nc12]

N-[(1H-benzimidazol-2-yl)methyl]-8-bromo-2-(methanesulfonyl)pyrazolo[1,5-a][1,3,5]triazin-4-amine (Intermediate 9, 100 mg, 213 μmol) and piperidine (63 μL, 640 μmol) were provided in acetonitrile (2.0 mL), N,N-diisopropylethylamine (110 μL, 640 μmol) was added and the mixture was stirred for 3 h at 70° C. in a sealed tube. The mixture was diluted with water and extracted with ethyl acetate. The combined organic layers were dried, concentrated and purified by preparative HPLC (HT basic) to give 34.2 mg (34% yield) of the title compound.

LC-MS (Method 2): Rₜ=1.24 min; MS (ESIpos): m/z=427 [M+H]⁺

¹H-NMR (400 MHz, DMSO-d6) δ[ppm]: 1.352 (0.66), 1.506 (0.78), 1.516 (0.82), 2.075 (16.00), 2.518 (1.27), 2.523 (0.92), 2.540 (0.92), 3.625 (1.39), 3.871 (0.20), 4.849 (1.91), 7.116 (0.94), 7.131 (1.01), 7.405 (0.33), 7.522 (0.35), 7.995 (6.26), 8.009 (0.27), 9.019 (0.35), 12.250 (0.51).

### Example 47

### N-[(1H-benzimidazol-2-yl)methyl]-8-bromo-2-(pyrrolidin-1-yl)pyrazolo[1,5-a][1,3,5]triazin-4-amine

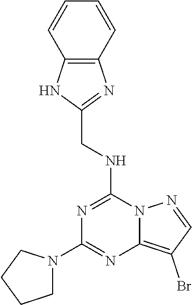 [SMILES: Brc1cnn2c(NCc3nc4ccccc4[nH]3)nc(N3CCCC3)nc12]

N-[(1H-benzimidazol-2-yl)methyl]-8-bromo-2-(methanesulfonyl)pyrazolo[1,5-a][1,3,5]triazin-4-amine (Intermediate 9, 100 mg, 213 μmol) and pyrrolidine (53 μL, 640 μmol) were provided in acetonitrile (2.0 mL), N,N-diisopropylethylamine (110 μL, 640 μmol) was added and the mixture was stirred for 19 h at 70° C. in a sealed tube. The mixture was diluted with water and extracted with ethyl acetate. The combined organic layers were dried, concentrated and purified by preparative HPLC (HT basic) to give 41.4 mg (43% yield) of the title compound.

LC-MS (Method 2): Rₜ=1.14 min; MS (ESIpos): m/z=413 [M+H]⁺

¹H-NMR (400 MHz, DMSO-d6) δ[ppm]: 1.811 (2.66), 1.835 (2.64), 2.326 (1.04), 2.331 (0.74), 2.518 (3.56), 2.522 (2.43), 2.539 (0.45), 2.669 (1.04), 2.673 (0.74), 3.445 (2.48), 4.878 (6.90), 7.120 (3.04), 7.134 (3.24), 7.400 (1.17), 7.415 (1.15), 7.526 (1.22), 7.986 (16.00), 8.000 (0.84), 8.988 (1.29), 12.234 (1.71).

### Example 48

### N-[(1H-benzimidazol-2-yl)methyl]-8-bromo-2-(2-oxa-6-azaspiro[3.3]heptan-6-yl)pyrazolo[1,5-a][1,3,5]triazin-4-amine

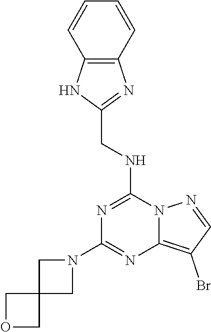 [SMILES: Brc1cnn2c(NCc3nc4ccccc4[nH]3)nc(N3CC4(COC4)C3)nc12]

N-[(1H-benzimidazol-2-yl)methyl]-8-bromo-2-(methanesulfonyl)pyrazolo[1,5-a][1,3,5]triazin-4-amine (Intermediate 9, 100 mg, 213 μmol) and 2-oxa-6-azaspiro[3.3]heptane (63.4 mg, 639 μmol) were provided in acetonitrile (2.0 mL), N,N-diisopropylethylamine (110 μL, 640 μmol) was added and the mixture was stirred for 22 h at 70° C. in a sealed tube. The mixture was diluted with water and extracted with ethyl acetate. The combined organic layers were dried, concentrated and purified by preparative HPLC (HT basic) to give 22.5 mg (24% yield) of the title compound.

LC-MS (Method 2): Rₜ=0.90 min; MS (ESIneg): m/z=439 [M−H]⁻

¹H-NMR (400 MHz, DMSO-d6) δ[ppm]: 2.074 (2.70), 2.327 (0.93), 2.331 (0.68), 2.518 (3.06), 2.523 (2.15), 2.540 (0.47), 2.669 (0.95), 2.673 (0.64), 4.132 (4.51), 4.641 (16.00), 4.869 (5.78), 7.133 (2.59), 7.146 (2.72), 7.411 (1.06), 7.427 (1.00), 7.535 (1.06), 8.028 (14.72), 9.095 (0.88), 12.224 (1.12).

### Example 49

### 8-bromo-N-[(5-phenyl-4H-1,2,4-triazol-3-yl)methyl]-2-(piperidin-1-yl)pyrazolo[1,5-a][1,3,5]triazin-4-amine

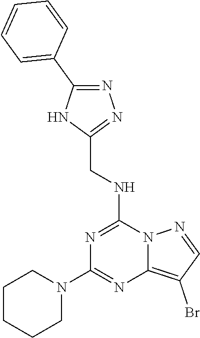 [SMILES: Brc1cnn2c(NCc3nnc(-c4ccccc4)[nH]3)nc(N3CCCCC3)nc12]

8-bromo-2-(methanesulfonyl)-N-[(5-phenyl-4H-1,2,4-triazol-3-yl)methyl]pyrazolo[1,5-a][1,3,5]triazin-4-amine (Intermediate 24, 100 mg, 223 μmol) and piperidine (66 μL, 670 μmol) were provided in acetonitrile (2.0 mL), N,N-diisopropylethylamine (120 μL, 670 μmol) was added and the mixture was stirred for 22 h at 70° C. in a sealed tube. The mixture was diluted with water and extracted with ethyl acetate. The combined organic layers were dried, concentrated and purified by preparative HPLC (HT basic) to give 53.2 mg (51% yield) of the title compound.

LC-MS (Method 2): Rₜ=1.14 min; MS (ESIpos): m/z=454 [M+H]⁺

¹H-NMR (400 MHz, DMSO-d6) δ[ppm]: 1.402 (4.14), 1.534 (3.01), 1.545 (3.06), 2.327 (1.33), 2.331 (0.94), 2.518 (4.94), 2.523 (3.29), 2.540 (1.08), 2.669 (1.36), 2.673 (0.94), 3.662 (6.34), 4.748 (2.39), 7.447 (2.78), 7.466 (2.94), 7.951 (6.25), 7.968 (5.72), 7.980 (16.00), 7.993 (0.57), 9.009 (0.78).

### Example 50

### 8-bromo-N-{[5-(3-methoxyphenyl)-4H-1,2,4-triazol-3-yl]methyl}-2-(morpholin-4-yl)pyrazolo[1,5-a][1,3,5]triazin-4-amine

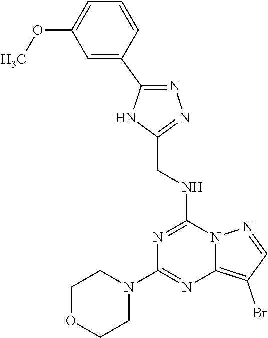 [SMILES: COc1cccc(-c2nnc(CNc3nc(N4CCOCC4)nc4c(Br)cnn34)[nH]2)c1]

2-{[8-bromo-2-(morpholin-4-yl)pyrazolo[1,5-a][1,3,5]triazin-4-yl]amino}acetohydrazide (Intermediate 39, 80.0 mg, 216 μmol) and 3-methoxybenzene-1-carboximidamide hydrogen chloride (1/1) (48.3 mg, 259 μmol) were dissolved in DMF (2.5 mL), sodium ethanolate (29.3 mg, 431 μmol) was added and the mixture was stirred for 45 min at 180° C. in a microwave. The mixture was filtered and purified by preparative HPLC (HT basic) to give 42.1 mg (40% yield) of the title compound.

LC-MS (Method 2): Rₜ=0.85 min; MS (ESIpos): m/z=486 [M+H]⁺

¹H-NMR (400 MHz, DMSO-d6) δ[ppm]: 2.336 (0.23), 2.518 (3.35), 2.523 (2.14), 3.548 (2.46), 3.621 (0.21), 3.654 (2.48), 3.665 (2.98), 3.803 (16.00), 3.812 (1.19), 4.762 (1.39), 6.987 (0.54), 7.006 (0.58), 7.363 (0.47), 7.383 (0.89), 7.402 (0.52), 7.490 (1.39), 7.493 (1.79), 7.495 (1.64), 7.499 (1.56), 7.536 (1.34), 7.556 (1.06), 8.021 (6.64), 9.112 (0.25).

### Example 51

### 8-bromo-N-{[5-(3-chlorophenyl)-4H-1,2,4-triazol-3-yl]methyl}-2-(morpholin-4-yl)pyrazolo[1,5-a][1,3,5]triazin-4-amine

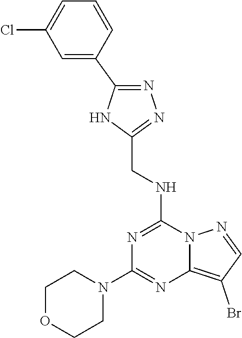 [SMILES: Clc1cccc(-c2nnc(CNc3nc(N4CCOCC4)nc4c(Br)cnn34)[nH]2)c1]

2-{[8-bromo-2-(morpholin-4-yl)pyrazolo[1,5-a][1,3,5]triazin-4-yl]amino}acetohydrazide (Intermediate 39, 80.0 mg, 216 μmol) and 3-chlorobenzene-1-carboximidamide hydrogen chloride (1/1) (49.4 mg, 259 μmol) were dissolved in DMF (2.5 mL), sodium ethanolate (29.3 mg, 431 μmol) was added and the mixture was stirred for 45 min at 180° C. in a microwave. The mixture was filtered and purified by preparative HPLC (HT basic) to give 24.6 mg (23% yield) of the title compound.

LC-MS (Method 2): Rₜ=0.87 min; MS (ESIpos): m/z=490 [M+H]⁺

¹H-NMR (400 MHz, DMSO-d6) δ[ppm]: 2.331 (1.21), 2.518 (6.61), 2.523 (4.48), 2.669 (1.72), 2.673 (1.24), 3.545 (6.52), 3.643 (7.04), 3.655 (8.24), 4.785 (5.63), 7.495 (6.75), 7.509 (3.76), 7.529 (0.80), 7.911 (2.13), 7.915 (2.90), 7.921 (2.41), 7.926 (2.01), 7.929 (2.53), 7.932 (2.21), 7.936 (2.44), 7.954 (6.03), 8.026 (16.00), 9.150 (0.80).

### Example 52

### 8-bromo-2-(morpholin-4-yl)-N-({5-[3-(trifluoromethyl)phenyl]-4H-1,2,4-triazol-3-yl}methyl)pyrazolo[1,5-a][1,3,5]triazin-4-amine

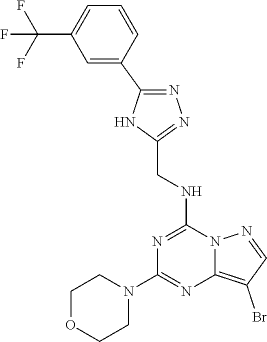 [SMILES: FC(F)(F)c1cccc(-c2nnc(CNc3nc(N4CCOCC4)nc4c(Br)cnn34)[nH]2)c1]

2-{[8-bromo-2-(morpholin-4-yl)pyrazolo[1,5-a][1,3,5]triazin-4-yl]amino}acetohydrazide (Intermediate 39, 80.0 mg, 216 μmol) and 3-(trifluoromethyl)benzene-1-carboximidamide hydrogen chloride (1/1) (58.1 mg, 259 μmol) were dissolved in DMF (2.5 mL), sodium ethanolate (29.3 mg, 431 μmol) was added and the mixture was stirred for 45 min at 180° C. in a microwave. The mixture was filtered and purified by preparative HPLC (HT basic) to give 53.1 mg (46% yield) of the title compound.

LC-MS (Method 2): Rₜ=0.92 min; MS (ESIpos): m/z=524 [M+H]⁺

¹H-NMR (400 MHz, DMSO-d6) δ[ppm]: 2.518 (6.83), 2.523 (4.48), 3.542 (5.28), 3.642 (5.77), 3.654 (6.80), 4.807 (3.89), 7.703 (1.08), 7.723 (2.65), 7.742 (1.86), 7.789 (2.47), 7.808 (1.47), 8.029 (16.00), 8.043 (0.33), 8.240 (3.97), 8.251 (3.01), 8.271 (2.47), 8.345 (0.18), 8.363 (0.21), 9.174 (1.03), 14.181 (0.31).

### Example 53

### 8-bromo-2-(morpholin-4-yl)-N-{[5-(pyridin-3-yl)-4H-1,2,4-triazol-3-yl]methyl}pyrazolo[1,5-a][1,3,5]triazin-4-amine

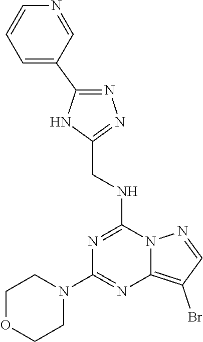 [SMILES: Brc1cnn2c(NCc3nnc(-c4cccnc4)[nH]3)nc(N3CCOCC3)nc12]

2-{[8-bromo-2-(morpholin-4-yl)pyrazolo[1,5-a][1,3,5]triazin-4-yl]amino}acetohydrazide (Intermediate 39, 80.0 mg, 216 μmol) and pyridine-3-carboximidamide hydrogen chloride (1/1) (40.8 mg, 259 μmol) were dissolved in DMF (2.5 mL), sodium ethanolate (29.3 mg, 431 μmol) was added and the mixture was stirred for 45 min at 180° C. in a microwave. The mixture was filtered and purified by preparative HPLC (HT basic) to give 17.8 mg (18% yield) of the title compound.

LC-MS (Method 2): Rₜ=0.63 min; MS (ESIpos): m/z=457 [M+H]⁺

¹H-NMR (400 MHz, DMSO-d6) δ[ppm]: 1.903 (0.85), 2.327 (2.74), 2.331 (1.89), 2.518 (8.81), 2.523 (6.16), 2.669 (2.74), 2.673 (1.93), 3.544 (6.02), 3.643 (6.74), 3.654 (7.78), 4.806 (8.18), 7.483 (2.07), 7.496 (2.20), 7.503 (2.16), 7.516 (2.11), 8.028 (16.00), 8.043 (0.49), 8.268 (2.38), 8.273 (3.42), 8.278 (2.34), 8.289 (2.11), 8.294 (3.28), 8.298 (2.16), 8.605 (3.46), 8.609 (3.42), 8.617 (3.42), 8.621 (3.10), 9.135 (5.30), 9.138 (5.26), 9.140 (5.03).

### Example 54

### 8-bromo-2-(morpholin-4-yl)-N-{[5-(pyridin-2-yl)-4H-1,2,4-triazol-3-yl]methyl}pyrazolo[1,5-a][1,3,5]triazin-4-amine

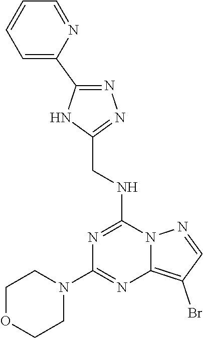 [SMILES: Brc1cnn2c(NCc3nnc(-c4ccccn4)[nH]3)nc(N3CCOCC3)nc12]

2-{[8-bromo-2-(morpholin-4-yl)pyrazolo[1,5-a][1,3,5]triazin-4-yl]amino}acetohydrazide (Intermediate 39, 80.0 mg, 216 μmol) and pyridine-2-carboximidamide hydrogen chloride (1/1) (40.8 mg, 259 μmol) were dissolved in DMF (2.5 mL), sodium ethanolate (29.3 mg, 431 μmol) was added and the mixture was stirred for 45 min at 180° C. in a microwave. The mixture was filtered and purified by preparative HPLC (HT basic) to give 20.6 mg (19% yield) of the title compound.

LC-MS (Method 1): Rₜ=0.93 min; MS (ESIpos): m/z=457 [M+H]⁺

¹H-NMR (400 MHz, DMSO-d6) δ[ppm]: 2.075 (0.57), 2.336 (0.34), 2.518 (4.44), 2.523 (3.03), 2.679 (0.34), 3.545 (4.61), 3.660 (5.16), 3.673 (6.15), 3.684 (3.66), 3.797 (0.17), 4.744 (2.74), 7.472 (0.78), 7.488 (1.12), 7.501 (0.88), 7.929 (0.61), 7.948 (1.39), 7.967 (0.93), 7.991 (0.19), 8.008 (3.14), 8.015 (16.00), 8.027 (1.81), 8.666 (1.83), 8.678 (1.83), 9.079 (0.74), 14.452 (0.17).

### Example 55

### 3-[5-({[8-bromo-2-(morpholin-4-yl)pyrazolo[1,5-a][1,3,5]triazin-4-yl]amino}methyl)-4H-1,2,4-triazol-3-yl]benzonitrile

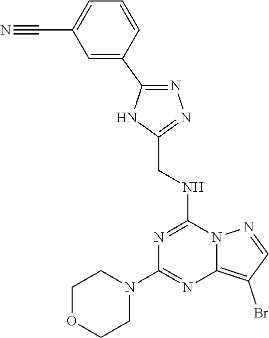 [SMILES: N#Cc1cccc(-c2nnc(CNc3nc(N4CCOCC4)nc4c(Br)cnn34)[nH]2)c1]

2-{[8-bromo-2-(morpholin-4-yl)pyrazolo[1,5-a][1,3,5]triazin-4-yl]amino}acetohydrazide (Intermediate 39, 80.0 mg, 216 μmol) and 3-cyanobenzene-1-carboximidamide (37.5 mg, 259 μmol) were dissolved in DMF (2.5 mL), sodium ethanolate (29.3 mg, 431 μmol; CAS 141-52-6) were added and the mixture was stirred for 45 min at 180° C. in the microwave. The mixture was filtered and purified by preparative HPLC (HT basic) to give 26.5 mg (95% purity, 24% yield) of the title compound.

LC-MS (Method 2): Rₜ=0.73 min; MS (ESIpos): m/z=481 [M+H]⁺

¹H-NMR (400 MHz, DMSO-d6) δ[ppm]: 2.075 (1.15), 2.337 (0.75), 2.518 (10.15), 2.523 (6.96), 2.679 (0.80), 3.544 (5.05), 3.638 (5.41), 3.649 (6.29), 4.805 (4.43), 7.671 (1.64), 7.691 (3.55), 7.710 (2.04), 7.885 (2.44), 7.905 (2.08), 8.000 (0.44), 8.029 (16.00), 8.261 (1.82), 8.265 (3.01), 8.268 (2.39), 8.281 (1.46), 8.285 (3.28), 8.288 (2.93), 8.292 (3.86), 8.295 (5.01), 9.172 (0.75).

### Example 56

### N-[(1H-benzimidazol-2-yl)methyl]-8-bromo-2-[(3R)-3-methylmorpholin-4-yl]pyrazolo[1,5-a][1,3,5]triazin-4-amine

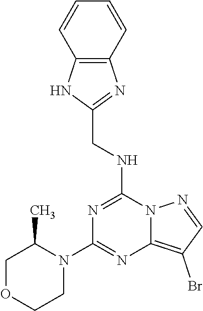 [SMILES: C[C@@H]1COCCN1c1nc(NCc2nc3ccccc3[nH]2)n2ncc(Br)c2n1]

N-[(1H-benzimidazol-2-yl)methyl]-8-bromo-2-(methanesulfonyl)pyrazolo[1,5-a][1,3,5]triazin-4-amine (Intermediate 9, 100 mg, 213 μmol) and (3R)-3-methylmorpholine (64.7 mg, 639 μmol) were provided in acetonitrile (1.8 mL), N,N-diisopropylethylamine (98 μL, 560 μmol) was added and the mixture was stirred for 24 h at 70° C. in a sealed tube. The mixture was diluted with water and extracted with ethyl acetate. The combined organic layers were dried, concentrated and purified by preparative HPLC (HT basic) to give 5.4 mg (5% yield) of the title compound.

LC-MS (Method 2): Rₜ=1.09 min; MS (ESIpos): m/z=443 [M+H]⁺

¹H-NMR (400 MHz, DMSO-d6) δ[ppm]: 0.936 (0.78), 1.137 (0.61), 1.232 (1.01), 1.905 (0.52), 2.318 (0.52), 2.322 (1.16), 2.326 (1.62), 2.331 (1.10), 2.336 (0.49), 2.518 (6.47), 2.522 (4.36), 2.539 (16.00), 2.660 (0.52), 2.664 (1.18), 2.668 (1.62), 2.673 (1.16), 2.678 (0.52), 3.000 (0.40), 3.008 (0.52), 3.031 (0.81), 3.039 (0.87), 3.064 (0.69), 3.074 (0.61), 3.264 (0.95), 3.403 (1.16), 3.410 (1.13), 3.431 (1.27), 3.438 (1.18), 3.552 (1.13), 3.580 (0.84), 3.779 (0.66), 3.807 (0.61), 4.151 (1.04), 4.181 (1.01), 4.494 (0.43), 4.856 (1.79), 7.116 (1.88), 7.129 (2.08), 7.385 (0.75), 7.400 (0.75), 7.519 (0.84), 7.536 (0.75), 8.034 (8.32), 9.134 (0.72), 12.259 (0.81).

### Example 57

### N-[(1H-benzimidazol-2-yl)methyl]-8-bromo-2-[(3S)-3-methylmorpholin-4-yl]pyrazolo[1,5-a][1,3,5]triazin-4-amine

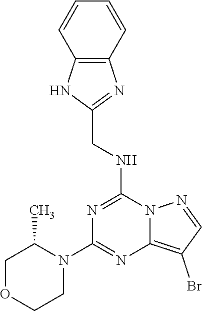 [SMILES: C[C@H]1COCCN1c1nc(NCc2nc3ccccc3[nH]2)n2ncc(Br)c2n1]

N-[(1H-benzimidazol-2-yl)methyl]-8-bromo-2-(methanesulfonyl)pyrazolo[1,5-a][1,3,5]triazin-4-amine (Intermediate 9, 100 mg, 213 μmol) and (3S)-3-methylmorpholine (64.7 mg, 639 μmol) were provided in acetonitrile (2.0 mL), N,N-diisopropylethylamine (120 μL, 670 μmol) was added and the mixture was stirred for 22 h at 70° C. in a sealed tube. The mixture was diluted with water and extracted with ethyl acetate. The combined organic layers were dried, concentrated and purified by preparative HPLC (HT basic) to give 4.6 mg (4% yield) of the title compound.

LC-MS (Method 2): Rₜ=1.08 min; MS (ESIpos): m/z=443 [M+H]⁺

¹H-NMR (400 MHz, DMSO-d6) δ[ppm]: 0.852 (0.44), 0.863 (0.48), 0.936 (1.21), 1.137 (1.21), 1.233 (1.33), 1.906 (0.60), 2.116 (0.48), 2.332 (1.65), 2.337 (0.72), 2.518 (10.41), 2.523 (6.95), 2.540 (15.32), 2.673 (1.65), 2.678 (0.72), 3.000 (0.64), 3.009 (0.80), 3.032 (1.25), 3.040 (1.33), 3.065 (1.05), 3.074 (0.92), 3.234 (0.76), 3.263 (1.29), 3.404 (1.49), 3.411 (1.53), 3.433 (1.81), 3.440 (1.69), 3.552 (1.73), 3.581 (1.21), 3.783 (0.96), 3.807 (0.92), 4.152 (1.61), 4.181 (1.53), 4.496 (0.68), 4.856 (3.42), 7.108 (1.89), 7.116 (2.93), 7.130 (3.22), 7.138 (2.17), 7.384 (1.33), 7.401 (1.33), 7.520 (1.37), 7.537 (1.21), 8.034 (16.00), 8.049 (0.72), 8.205 (0.44), 8.235 (0.44), 9.135 (1.29), 12.257 (1.69).

### Example 58

### N-[(1H-benzimidazol-2-yl)methyl]-8-bromo-2-(2,2-dimethylmorpholin-4-yl)pyrazolo[1,5-a][1,3,5]triazin-4-amine

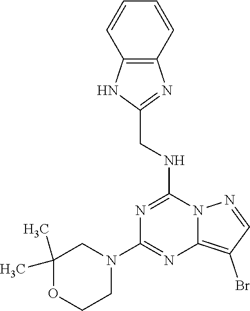 [SMILES: CC1(C)CN(c2nc(NCc3nc4ccccc4[nH]3)n3ncc(Br)c3n2)CCO1]

N-[(1H-benzimidazol-2-yl)methyl]-8-bromo-2-(methanesulfonyl)pyrazolo[1,5-a][1,3,5]triazin-4-amine (Intermediate 9, 100 mg, 213 μmol) and 2,2-dimethylmorpholine (73.6 mg, 639 μmol) were provided in acetonitrile (2.0 mL), N,N-diisopropylethylamine (110 μL, 640 μmol) was added and the mixture was stirred for 5 h at 70° C. in a sealed tube. The mixture was diluted with water and extracted with ethyl acetate. The combined organic layers were dried, concentrated and purified by preparative HPLC (HT basic) to give 49.5 mg (50% yield) of the title compound.

LC-MS (Method 2): Rₜ=1.10 min; MS (ESIpos): m/z=457 [M+H]⁺

¹H-NMR (400 MHz, DMSO-d6) δ[ppm]: 0.759 (1.42), 1.105 (0.53), 2.518 (5.52), 2.522 (3.52), 3.385 (1.11), 3.506 (1.60), 3.604 (1.94), 4.824 (2.51), 7.093 (0.80), 7.106 (2.76), 7.111 (2.58), 7.115 (3.12), 7.122 (4.34), 7.129 (3.47), 7.134 (2.56), 7.138 (2.80), 7.152 (0.82), 7.373 (2.05), 7.389 (1.71), 7.513 (2.11), 7.518 (2.09), 7.535 (2.00), 8.026 (16.00), 8.040 (0.56), 9.144 (1.67), 12.275 (2.92).

### Example 59

### [(2S)-1-(4-{[(1H-benzimidazol-2-yl)methyl]amino}-8-bromopyrazolo[1,5-a][1,3,5]triazin-2-yl)piperidin-2-yl]methanol

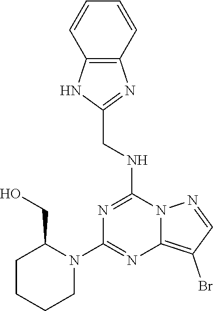 [SMILES: OC[C@@H]1CCCCN1c1nc(NCc2nc3ccccc3[nH]2)n2ncc(Br)c2n1]

N-[(1H-benzimidazol-2-yl)methyl]-8-bromo-2-(methanesulfonyl)pyrazolo[1,5-a][1,3,5]triazin-4-amine (Intermediate 9, 100 mg, 213 μmol) and [(2S)-piperidin-2-yl]methanol (73.6 mg, 639 μmol) were provided in acetonitrile (2.0 mL), N,N-diisopropylethylamine (110 μL, 640 μmol) was added and the mixture was stirred for 5 h at 70° C. in a sealed tube. The mixture was diluted with water and extracted with ethyl acetate. The combined organic layers were dried, concentrated and purified by preparative HPLC (HT basic) to give 30.9 mg (31% yield) of the title compound.

Specific Optical Rotation (Method O1): +17.5° (c=10 mg/mL, DMSO)

LC-MS (Method 2): Rₜ=1.09 min; MS (ESIpos): m/z=457 [M+H]⁺

¹H-NMR (400 MHz, DMSO-d6) δ[ppm]: 1.232 (0.43), 1.327 (0.65), 1.359 (1.18), 1.371 (0.91), 1.393 (0.86), 1.495 (1.59), 1.529 (1.26), 1.562 (0.83), 1.762 (0.81), 1.793 (0.73), 1.902 (0.46), 2.331 (1.08), 2.336 (0.48), 2.518 (5.65), 2.522 (3.82), 2.539 (0.59), 2.673 (1.10), 2.678 (0.51), 2.822 (0.51), 3.530 (1.37), 4.852 (3.82), 4.865 (2.88), 7.100 (0.62), 7.115 (2.31), 7.119 (1.99), 7.125 (2.88), 7.131 (3.23), 7.138 (3.15), 7.143 (2.04), 7.148 (2.45), 7.162 (0.67), 7.408 (1.67), 7.426 (1.51), 7.528 (1.86), 7.549 (1.72), 7.985 (16.00), 8.000 (0.46), 9.009 (0.97), 12.190 (0.62).

### Example 60

### [(2R)-1-(4-{[(1H-benzimidazol-2-yl)methyl]amino}-8-bromopyrazolo[1,5-a][1,3,5]triazin-2-yl)piperidin-2-yl]methanol

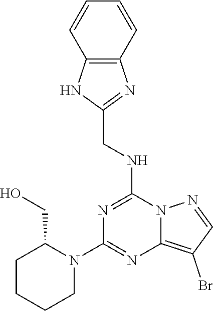 [SMILES: OC[C@H]1CCCCN1c1nc(NCc2nc3ccccc3[nH]2)n2ncc(Br)c2n1]

N-[(1H-benzimidazol-2-yl)methyl]-8-bromo-2-(methanesulfonyl)pyrazolo[1,5-a][1,3,5]triazin-4-amine (Intermediate 9, 100 mg, 213 μmol) and [(2R)-piperidin-2-yl]methanol (73.6 mg, 639 μmol) were provided in acetonitrile (2.0 mL), N,N-diisopropylethylamine (110 μL, 640 μmol) was added and the mixture was stirred for 5 h at 70° C. in a sealed tube. The mixture was diluted with water and extracted with ethyl acetate. The combined organic layers were dried, concentrated and purified by preparative HPLC (HT basic) to give 34.3 mg (35% yield) of the title compound. Specific Optical Rotation (Method O1): −14.0° (c=10 mg/mL, DMSO) LC-MS (Method 2): Rₜ=1.09 min; MS (ESIpos): m/z=457 [M+H]⁺

¹H-NMR (400 MHz, DMSO-d6) δ[ppm]: 1.328 (0.60), 1.347 (0.86), 1.360 (1.14), 1.373 (0.88), 1.394 (0.83), 1.405 (0.57), 1.497 (1.52), 1.530 (1.17), 1.561 (0.79), 1.763 (0.79), 1.791 (0.69), 2.337 (0.43), 2.457 (0.50), 2.518 (5.67), 2.523 (3.81), 2.679 (0.45), 2.821 (0.48), 3.533 (1.29), 4.853 (3.50), 4.865 (2.79), 7.101 (0.69), 7.115 (2.45), 7.119 (2.05), 7.126 (2.81), 7.132 (3.71), 7.138 (3.14), 7.145 (2.14), 7.148 (2.62), 7.162 (0.76), 7.408 (1.81), 7.425 (1.57), 7.528 (1.98), 7.532 (2.05), 7.549 (1.90), 7.986 (16.00), 9.008 (1.07), 12.185 (0.60).

### Example 61

### 8-bromo-N-{[5-(2-methoxyphenyl)-4H-1,2,4-triazol-3-yl]methyl}-2-(morpholin-4-yl)pyrazolo[1,5-a][1,3,5]triazin-4-amine

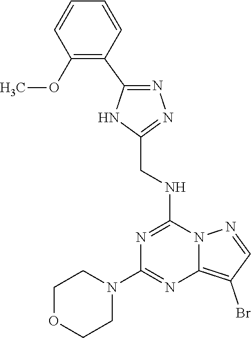 [SMILES: COc1ccccc1-c1nnc(CNc2nc(N3CCOCC3)nc3c(Br)cnn23)[nH]1]

2-{[8-bromo-2-(morpholin-4-yl)pyrazolo[1,5-a][1,3,5]triazin-4-yl]amino}acetohydrazide (Intermediate 39, 80.0 mg, 216 μmol) and 2-methoxybenzene-1-carboximidamide hydrogen chloride (1/1) (48.3 mg, 259 μmol) were dissolved in DMF (2.5 mL), sodium ethanolate (29.3 mg, 431 μmol) was added and the mixture was stirred for 45 min at 180° C. in a microwave. The mixture was filtered and purified by preparative HPLC (HT basic) to give 27.2 mg (24% yield) of the title compound.

LC-MS (Method 2): Rₜ=1.09 min; MS (ESIpos): m/z=486 [M+H]⁺

¹H-NMR (400 MHz, DMSO-d6) δ[ppm]: 2.074 (2.44), 2.337 (0.43), 2.518 (5.15), 2.523 (3.47), 2.679 (0.38), 3.300 (0.60), 3.550 (4.99), 3.669 (5.53), 3.682 (6.56), 3.693 (3.91), 3.933 (9.55), 4.710 (3.31), 7.047 (1.30), 7.065 (2.66), 7.083 (1.46), 7.173 (2.28), 7.194 (2.66), 7.432 (1.25), 7.437 (1.30), 7.454 (1.84), 7.471 (0.98), 7.476 (0.92), 7.996 (1.03), 8.007 (16.00), 8.020 (0.98), 9.017 (0.87), 13.527 (0.87).

### Example 62

### N-[(1H-benzimidazol-2-yl)methyl]-8-bromo-2-(4-methoxypiperidin-1-yl)pyrazolo[1,5-a][1,3,5]triazin-4-amine

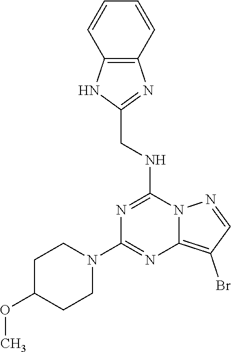 [SMILES: COC1CCN(c2nc(NCc3nc4ccccc4[nH]3)n3ncc(Br)c3n2)CC1]

N-[(1H-benzimidazol-2-yl)methyl]-8-bromo-2-(methanesulfonyl)pyrazolo[1,5-a][1,3,5]triazin-4-amine (Intermediate 9, 100 mg, 213 μmol) and 4-methoxypiperidine (73.6 mg, 639 μmol) were provided in acetonitrile (2.0 mL), N,N-diisopropylethylamine (110 μL, 640 μmol) was added and the mixture was stirred for 5 h at 70° C. in a sealed tube. The mixture was diluted with water and extracted with ethyl acetate. The combined organic layers were dried, concentrated and purified by preparative HPLC (HT basic) to give 41.3 mg (41% yield) of the title compound.

LC-MS (Method 2): Rₜ=1.10 min; MS (ESIneg): m/z=455 [M−H]⁻

¹H-NMR (400 MHz, DMSO-d6) δ[ppm]: 2.075 (1.87), 2.518 (1.28), 2.523 (0.86), 2.540 (0.77), 3.196 (16.00), 3.242 (0.45), 3.312 (0.46), 4.843 (1.31), 4.856 (1.31), 7.109 (0.93), 7.114 (0.79), 7.119 (1.00), 7.126 (1.57), 7.132 (1.10), 7.137 (0.81), 7.142 (1.01), 7.386 (0.76), 7.393 (0.49), 7.402 (0.72), 7.407 (0.60), 7.522 (0.66), 7.527 (0.69), 7.544 (0.63), 8.009 (6.48), 9.068 (0.53), 12.244 (0.93).

### Example 63

### methyl 1-(4-{[(1H-benzimidazol-2-yl)methyl]amino}-8-bromopyrazolo[1,5-a][1,3,5]triazin-2-yl)piperidine-4-carboxylate

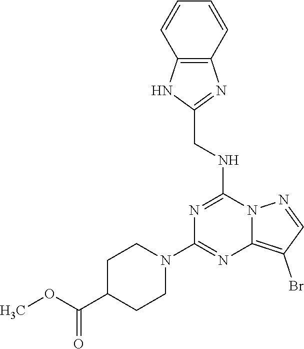 [SMILES: COC(=O)C1CCN(c2nc(NCc3nc4ccccc4[nH]3)n3ncc(Br)c3n2)CC1]

N-[(1H-benzimidazol-2-yl)methyl]-8-bromo-2-(methanesulfonyl)pyrazolo[1,5-a][1,3,5]triazin-4-amine (Intermediate 9, 200 mg, 426 μmol) and methyl piperidine-4-carboxylate (183 mg, 1.28 mmol) were provided in acetonitrile (4.0 mL), N,N-diisopropylethylamine (220 μL, 1.3 mmol) was added and the mixture was stirred for 22 h at 70° C. in a sealed tube. The mixture was diluted with water and extracted with ethyl acetate. The combined organic layers were dried, concentrated and purified by preparative HPLC (HT basic) to give 19.9 mg (9% yield) of the title compound.

LC-MS (Method 2): Rₜ=1.12 min; MS (ESIpos): m/z=485 [M+H]⁺

¹H-NMR (400 MHz, DMSO-d6) δ[ppm]: 1.233 (0.25), 1.294 (0.29), 1.352 (0.22), 1.699 (0.32), 1.907 (0.19), 2.326 (0.82), 2.331 (0.57), 2.336 (0.26), 2.518 (3.08), 2.522 (2.05), 2.539 (0.65), 2.548 (0.69), 2.557 (0.39), 2.565 (0.22), 2.575 (0.29), 2.668 (0.80), 2.673 (0.58), 2.678 (0.25), 2.892 (0.47), 2.920 (0.83), 2.948 (0.48), 3.571 (16.00), 4.391 (0.47), 4.845 (2.26), 4.860 (2.22), 7.113 (1.16), 7.128 (1.25), 7.380 (0.42), 7.392 (0.43), 7.521 (0.46), 8.018 (7.74), 9.074 (0.57), 9.089 (1.21), 9.103 (0.53), 12.242 (0.94).

### Example 64

### 8-bromo-N-[(5-phenyl-4H-1,2,4-triazol-3-yl)methyl]-2-(pyrrolidin-1-yl)pyrazolo[1,5-a][1,3,5]triazin-4-amine

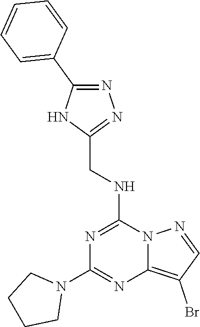 [SMILES: Brc1cnn2c(NCc3nnc(-c4ccccc4)[nH]3)nc(N3CCCC3)nc12]

8-bromo-2-(methanesulfonyl)-N-[(5-phenyl-4H-1,2,4-triazol-3-yl)methyl]pyrazolo[1,5-a][1,3,5]triazin-4-amine (Intermediate 24, 100 mg, 200 μmol) and pyrrolidine (50 μL, 600 μmol) were provided in acetonitrile (2.0 mL), N,N-diisopropylethylamine (100 μL, 600 μmol) was added and the mixture was stirred for 5 h at 70° C. in a sealed tube. The mixture was diluted with water and extracted with ethyl acetate. The combined organic layers were dried, concentrated and purified by preparative HPLC (HT basic) to give 67.2 mg (74% yield) of the title compound.

LC-MS (Method 1): Rₜ=1.17 min; MS (ESIneg): m/z=438 [M−H]⁻

¹H-NMR (400 MHz, DMSO-d6) δ[ppm]: 1.841 (2.72), 1.903 (0.24), 2.332 (0.68), 2.336 (0.31), 2.518 (3.78), 2.522 (2.53), 2.539 (0.94), 2.673 (0.70), 2.678 (0.29), 3.427 (2.29), 3.443 (2.32), 4.770 (2.07), 4.779 (2.09), 7.446 (1.86), 7.463 (2.03), 7.951 (3.50), 7.954 (4.47), 7.971 (16.00), 7.986 (0.75), 8.969 (0.37).

### Example 65

### 8-bromo-2-(2-oxa-6-azaspiro[3.3]heptan-6-yl)-N-[(5-phenyl-4H-1,2,4-triazol-3-yl)methyl]pyrazolo[1,5-a][1,3,5]triazin-4-amine

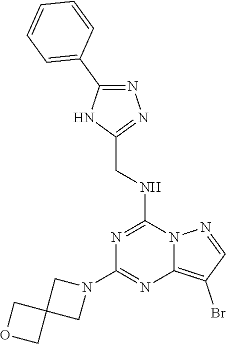 [SMILES: Brc1cnn2c(NCc3nnc(-c4ccccc4)[nH]3)nc(N3CC4(COC4)C3)nc12]

8-bromo-2-(methanesulfonyl)-N-[(5-phenyl-4H-1,2,4-triazol-3-yl)methyl]pyrazolo[1,5-a][1,3,5]triazin-4-amine (Intermediate 24, 100 mg, 200 μmol) and 2-oxa-6-azaspiro[3.3]heptane (59.6 mg, 601 μmol) were provided in acetonitrile (2.0 mL), N,N-diisopropylethylamine (100 μL, 600 μmol) was added and the mixture was stirred for 6 h at 70° C. in a sealed tube. The mixture was diluted with water and the precipitate was isolated by filtration, washed with water and dried to give 85.6 mg (85% yield) of the title compound.

LC-MS (Method 2): Rₜ=0.76 min; MS (ESIpos): m/z=468 [M+H]⁺

¹H-NMR (400 MHz, DMSO-d6) δ[ppm]: 2.336 (0.22), 2.518 (2.83), 2.523 (1.94), 2.679 (0.24), 3.281 (0.37), 4.155 (7.07), 4.525 (0.25), 4.659 (16.00), 4.769 (5.49), 7.405 (0.44), 7.423 (1.55), 7.430 (0.66), 7.441 (1.89), 7.455 (3.12), 7.473 (3.72), 7.490 (1.36), 7.495 (0.91), 7.953 (0.51), 7.959 (3.67), 7.962 (4.99), 7.966 (2.24), 7.974 (1.27), 7.979 (4.40), 7.983 (3.46), 7.994 (0.24), 8.002 (0.17), 8.013 (12.67), 8.027 (0.65), 9.049 (0.22).

### Example 66

### 8-bromo-N-{[5-(4-fluorophenyl)-1H-imidazol-2-yl]methyl}-2-(morpholin-4-yl)pyrazolo[1,5-a][1,3,5]triazin-4-amine

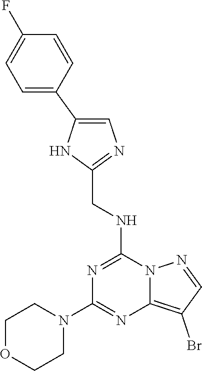 [SMILES: Fc1ccc(-c2cnc(CNc3nc(N4CCOCC4)nc4c(Br)cnn34)[nH]2)cc1]

{[8-bromo-2-(morpholin-4-yl)pyrazolo[1,5-a][1,3,5]triazin-4-yl]amino}ethanimidamide hydrogen chloride (1/1) (Intermediate 48, 80.0 mg, 204 μmol) and 2-bromo-1-(4-fluorophenyl)ethan-1-one (57.6 mg, 266 μmol) were dissolved in acetonitrile (2.3 mL), potassium carbonate (113 mg, 817 μmol) was added and the mixture was stirred in a sealed tube over night at 90° C. The mixture was concentrated, water and ethyl acetate were added and the organic layer was separated, dried with sodium sulfate, filtered and concentrated. The remaining material was purified by preparative HPLC (HT basic) to give 9.8 mg (9% yield) of the title compound.

LC-MS (Method 2): Rₜ=1.14 min; MS (ESIneg): m/z=471 [M−H]⁻

¹H-NMR (400 MHz, DMSO-d6) δ[ppm]: 2.210 (0.50), 2.518 (4.95), 2.523 (3.41), 3.567 (3.70), 3.577 (2.94), 3.674 (4.52), 3.687 (4.95), 3.698 (2.73), 4.693 (2.83), 4.707 (2.62), 7.122 (2.44), 7.128 (0.86), 7.140 (1.00), 7.145 (4.99), 7.150 (1.04), 7.162 (0.86), 7.167 (2.58), 7.231 (0.47), 7.238 (0.43), 7.501 (3.23), 7.506 (3.16), 7.733 (2.51), 7.739 (1.08), 7.747 (2.73), 7.755 (2.73), 7.764 (0.93), 7.769 (2.37), 8.007 (16.00), 8.021 (0.50), 8.977 (0.57), 8.991 (1.11), 9.004 (0.54), 11.939 (1.18).

### Example 67

### (4-{4-[(1H-benzimidazol-2-ylmethyl)amino]-8-bromopyrazolo[1,5-a][1,3,5]triazin-2-yl}morpholin-3-yl)methanol

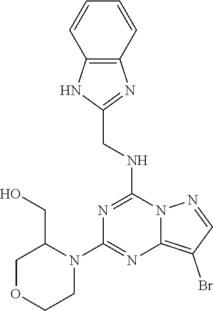 [SMILES: OCC1COCCN1c1nc(NCc2nc3ccccc3[nH]2)n2ncc(Br)c2n1]

N-[(1H-benzimidazol-2-yl)methyl]-8-bromo-2-(methanesulfonyl)pyrazolo[1,5-a][1,3,5]triazin-4-amine (Intermediate 9, 100 mg, 213 μmol) and morpholin-3-ylmethanol (74.9 mg, 639 μmol) were dissolved in acetonitrile (2.0 mL), N-ethyl-N-isopropylpropan-2-amine (110 μL, 639 μmol) was added and the mixture was stirred for 24 h at 70° C. The mixture was diluted with water and extracted with ethyl acetate. The combined organic layers were filtered, dried and concentrated. The remaining material was purified by preparative HPLC (HT basic) to give 5.90 mg (5% yield) of the title compound.

LC-MS (Method 2): Rₜ=0.94 min; MS (ESIpos): m/z=459 [M+H]⁺

¹H-NMR (400 MHz, DMSO-d6) δ[ppm]: 1.233 (1.30), 1.906 (0.55), 2.336 (0.65), 2.518 (9.15), 2.523 (5.93), 2.540 (3.49), 2.673 (1.44), 2.678 (0.65), 3.073 (0.51), 3.351 (3.19), 3.373 (2.36), 3.380 (2.16), 3.486 (0.62), 3.655 (0.96), 3.918 (0.93), 4.268 (0.58), 4.484 (1.03), 4.499 (1.78), 4.515 (0.93), 4.880 (2.36), 4.892 (2.64), 4.962 (1.10), 7.105 (0.93), 7.119 (2.84), 7.124 (2.54), 7.131 (3.15), 7.137 (4.56), 7.142 (3.53), 7.150 (2.57), 7.154 (3.05), 7.168 (0.99), 7.411 (2.19), 7.427 (1.92), 7.530 (2.36), 7.533 (2.47), 7.551 (2.33), 8.026 (16.00), 9.101 (1.16), 9.115 (2.19), 9.129 (1.10), 12.195 (0.69).

### Example 68

### 8-bromo-2-(2,2-dimethylmorpholin-4-yl)-N-[(5-phenyl-4H-1,2,4-triazol-3-yl)methyl]pyrazolo[1,5-a][1,3,5]triazin-4-amine

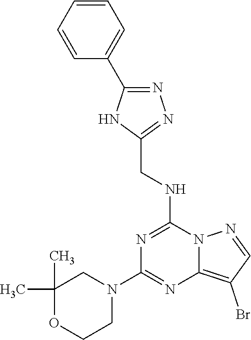 [SMILES: CC1(C)CN(c2nc(NCc3nnc(-c4ccccc4)[nH]3)n3ncc(Br)c3n2)CCO1]

8-bromo-2-(methanesulfonyl)-N-[(5-phenyl-4H-1,2,4-triazol-3-yl)methyl]pyrazolo[1,5-a][1,3,5]triazin-4-amine (Intermediate 24, 100 mg, 200 μmol) and 2,2-dimethylmorpholine (69.2 mg, 601 μmol) were suspended in acetonitrile (2.0 mL), N-ethyl-N-isopropylpropan-2-amine (100 μL, 600 μmol) was added and the mixture was stirred for 5 h at 70° C. in a sealed tube. The mixture was diluted with water and extracted with ethyl acetate. The combined organic layers were concentrated. The remaining material was purified by preparative HPLC (HT acidic) to give 31.2 mg (31% yield) of the title compound.

LC-MS (Method 1): Rₜ=1.16 min; MS (ESIpos): m/z=484 [M+H]⁺

¹H-NMR (400 MHz, DMSO-d6) δ[ppm]: 0.941 (2.15), 2.074 (0.64), 2.331 (0.90), 2.518 (4.25), 2.522 (2.83), 2.539 (1.62), 2.673 (0.90), 3.476 (2.87), 3.561 (3.33), 3.649 (3.31), 4.735 (2.56), 7.415 (1.45), 7.445 (3.22), 7.464 (3.59), 7.481 (1.47), 7.949 (6.55), 7.966 (5.90), 7.969 (4.65), 8.011 (16.00), 8.025 (0.42), 9.121 (0.79).

### Example 69

### 8-bromo-N-{[5-(2-chlorophenyl)-4H-1,2,4-triazol-3-yl]methyl}-2-(morpholin-4-yl)pyrazolo[1,5-a][1,3,5]triazin-4-amine

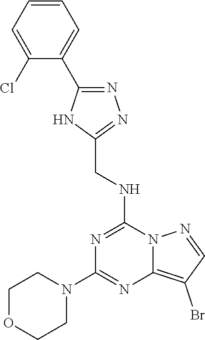 [SMILES: Clc1ccccc1-c1nnc(CNc2nc(N3CCOCC3)nc3c(Br)cnn23)[nH]1]

2-{[8-bromo-2-(morpholin-4-yl)pyrazolo[1,5-a][1,3,5]triazin-4-yl]amino}acetohydrazide (Intermediate 39, 80.0 mg, 216 μmol) and 2-chlorobenzene-1-carboximidamide-hydrogen chloride (1/1) (49.4 mg, 259 μmol) were dissolved in DMF (2.5 mL), sodium ethanolate (29.3 mg, 431 μmol; CAS 141-52-6) was added and the mixture was stirred in a sealed tube for 45 min at 180° C. in the microwave. The mixture was filtered and purified by preparative HPLC (HT basic) to give 5.0 mg (4% yield) of the title compound.

LC-MS (Method 2): Rₜ=0.88 min; MS (ESIpos): m/z=490 [M+H]⁺

¹H-NMR (400 MHz, DMSO-d6) δ[ppm]: 2.326 (1.94), 2.331 (1.35), 2.518 (7.30), 2.522 (4.56), 2.669 (1.94), 2.673 (1.39), 3.381 (0.93), 3.558 (6.46), 3.568 (5.91), 3.645 (6.63), 3.657 (7.35), 3.683 (1.48), 3.696 (1.39), 4.066 (0.89), 4.788 (6.21), 7.419 (0.84), 7.434 (2.28), 7.438 (2.70), 7.443 (1.94), 7.450 (3.76), 7.456 (2.83), 7.462 (2.07), 7.467 (1.98), 7.481 (0.80), 7.563 (2.74), 7.581 (1.86), 7.586 (1.86), 7.761 (2.11), 7.766 (1.94), 7.778 (1.65), 7.783 (1.82), 7.999 (2.45), 8.009 (0.63), 8.020 (16.00), 9.129 (0.51).

### Example 70

### [(2S)-1-(8-bromo-4-{[(5-phenyl-4H-1,2,4-triazol-3-yl)methyl]amino}pyrazolo[1,5-a][1,3,5]triazin-2-yl)piperidin-2-yl]methanol

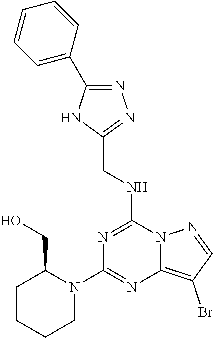 [SMILES: OC[C@@H]1CCCCN1c1nc(NCc2nnc(-c3ccccc3)[nH]2)n2ncc(Br)c2n1]

8-bromo-2-(methanesulfonyl)-N-[(5-phenyl-4H-1,2,4-triazol-3-yl)methyl]pyrazolo[1,5-a][1,3,5]triazin-4-amine (Intermediate 24, 100 mg, 200 μmol) and [(2S)-piperidin-2-yl]methanol (69 mg, 0.6 mmol) were suspended in acetonitrile (2.0 mL), N-ethyl-N-isopropylpropan-2-amine (104 μL, 0.6 mmol; CAS-RN:[7087-68-5]) was added and the mixture was stirred in a sealed tube for 22 h at 70° C. The mixture was diluted with water and extracted with ethyl acetate. The combined organic layers were concentrated. The remaining material was purified by preparative HPLC (HT acidic) to give 5.10 mg of the title compound.

In a second preparation 8-bromo-2-(methanesulfonyl)-N-[(5-phenyl-4H-1,2,4-triazol-3-yl)methyl]pyrazolo[1,5-a][1,3,5]triazin-4-amine (Intermediate 24, 200 mg, 400 μmol) and [(2S)-piperidin-2-yl]methanol (138 mg, 1.20 mmol) were suspended in acetonitrile (4.0 mL), N-ethyl-N-isopropylpropan-2-amine (210 μL, 1.2 mmol; CAS-RN:[7087-68-5]) was added and the mixture was stirred for 22 h at 70° C. The mixture was diluted with water and extracted with ethyl acetate. The combined organic layers were concentrated. The remaining material was purified by preparative HPLC (HT acidic) to give 12.9 mg of the title compound.

The two products were combined and further purified by preparative HPLC to give 7.1 mg (2% yield) of the title compound.

### Preparative HPLC Method

Instrument: Waters Autopurificationsystem; Column: Waters XBrigde C18 5μ 50×50 mm; eluent A: water+0.1 Vol-% formic acid, eluent B: acetonitrile; gradient: 0.00-0.50 min 18% B (50-100 mL/min), 0.51-8.50 min 18-38% B (100 mL/min), DAD scan: 210-400 nm.

### Analytical HPLC Method

Instrument: Waters Acquity UPLCMS SingleQuad; Column: Acquity UPLC BEH C18 1.7 μm, 50×2.1 mm; eluent A: water+0.1 Vol-% formic acid, eluent B: acetonitrile; gradient: 0-2.6 min 1-99% B, 2.6-3.0 min 99% B; flow 0.8 mL/min; temperature: 60° C.; DAD scan: 210-400 nm.

Analytical HPLC: Rₜ=1.12 min.

LC-MS (Method 1): Rₜ=1.13 min; MS (ESIpos): m/z=484 [M+H]⁺

¹H-NMR (400 MHz, DMSO-d6) δ[ppm]: −0.008 (3.95), 0.008 (3.70), 0.854 (0.45), 1.235 (3.57), 1.342 (1.21), 1.362 (1.78), 1.375 (2.42), 1.386 (1.85), 1.408 (1.66), 1.503 (3.12), 1.552 (2.61), 1.584 (2.10), 1.801 (2.04), 1.833 (1.72), 1.910 (0.57), 2.329 (3.57), 2.334 (2.49), 2.339 (1.15), 2.521 (13.51), 2.525 (8.73), 2.542 (4.40), 2.671 (3.57), 2.676 (2.61), 2.681 (1.21), 2.766 (0.70), 2.778 (0.83), 2.796 (1.15), 2.827 (1.98), 2.860 (1.08), 3.445 (2.04), 3.471 (3.31), 3.486 (2.87), 3.560 (1.59), 4.590 (1.40), 4.708 (1.85), 4.819 (4.08), 7.441 (3.57), 7.963 (7.08), 7.979 (16.00), 8.135 (0.38), 8.852 (0.51), 9.026 (1.34), 13.842 (1.15), 14.272 (0.70).

### Example 71

### [(2R)-1-(8-bromo-4-{[(5-phenyl-4H-1,2,4-triazol-3-yl)methyl]amino}pyrazolo[1,5-a][1,3,5]triazin-2-yl)piperidin-2-yl]methanol

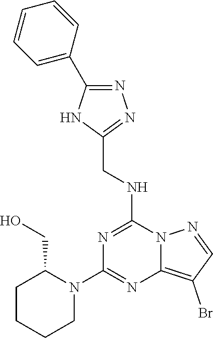 [SMILES: OC[C@H]1CCCCN1c1nc(NCc2nnc(-c3ccccc3)[nH]2)n2ncc(Br)c2n1]

8-bromo-2-(methanesulfonyl)-N-[(5-phenyl-4H-1,2,4-triazol-3-yl)methyl]pyrazolo[1,5-a][1,3,5]triazin-4-amine (Intermediate 24, 100 mg, 200 μmol) and [(2R)-piperidin-2-yl]methanol (69 mg, 0.6 mmol) were suspended in acetonitrile (2.0 mL), N-ethyl-N-isopropylpropan-2-amine (105 μL, 0.6 mmol; CAS-RN:[7087-68-5]) was added and the mixture was stirred for 22 h at 70° C. The mixture was diluted with water and extracted with ethyl acetate. The combined organic layers were concentrated. The remaining material was purified by preparative HPLC (HT acidic) to give 3.7 mg of the title compound.

In a second preparation 8-bromo-2-(methanesulfonyl)-N-[(5-phenyl-4H-1,2,4-triazol-3-yl)methyl]pyrazolo[1,5-a][1,3,5]triazin-4-amine (Intermediate 24, 200 mg, 400 μmol) and [(2R)-piperidin-2-yl]methanol (138 mg, 1.20 mmol) were suspended in acetonitrile (4.0 mL), N-ethyl-N-isopropylpropan-2-amine (210 μL, 1.2 mmol; CAS-RN:[7087-68-5]) was added and the mixture was stirred for 22 h at 70° C. The mixture was diluted with water and extracted with ethyl acetate. The combined organic layers were concentrated. The remaining material was purified by preparative HPLC (HT acidic) to give 12.9 mg of the title compound.

The two products were combined and further purified by preparative HPLC to give 8.2 mg (3% yield) of the title compound.

### Preparative HPLC Method

Instrument: Waters Autopurificationsystem; Column: Waters XBrigde C18 5μ50×50 mm; eluent A: water+0.1 Vol-% formic acid, eluent B: acetonitrile; gradient: 0.00-0.50 min 28% B (50-100 mL/min), 0.51-8.50 min 28-48% B (100 mL/min), DAD scan: 210-400 nm.

### Analytical HPLC Method

Instrument: Waters Acquity UPLCMS SingleQuad; Column: Acquity UPLC BEH C18 1.7 μm, 50×2.1 mm; eluent A: water+0.1 Vol-% formic acid, eluent B: acetonitrile; gradient: 0-2.6 min 1-99% B, 2.6-3.0 min 99% B; flow 0.8 mL/min; temperature: 60° C.; DAD scan: 210-400 nm.

Analytical HPLC: Rₜ=1.11 min.

LC-MS (Method 1): Rₜ=1.12 min; MS (ESIneg): m/z=482 [M−H]⁻

¹H-NMR (400 MHz, DMSO-d6) δ[ppm]: −0.008 (1.09), 0.008 (0.86), 1.234 (1.35), 1.342 (0.49), 1.363 (0.71), 1.375 (1.00), 1.387 (0.76), 1.408 (0.68), 1.506 (1.25), 1.553 (1.20), 1.585 (0.92), 1.801 (1.00), 1.833 (0.82), 2.088 (0.41), 2.329 (0.93), 2.334 (0.67), 2.521 (3.94), 2.525 (2.61), 2.542 (0.55), 2.671 (0.93), 2.676 (0.68), 2.796 (0.49), 2.825 (0.82), 2.858 (0.46), 3.445 (0.74), 3.471 (1.31), 3.488 (1.12), 3.559 (0.67), 3.991 (0.59), 4.576 (0.60), 4.776 (2.14), 4.827 (1.14), 7.422 (1.28), 7.451 (2.79), 7.470 (3.09), 7.487 (1.27), 7.960 (4.99), 7.978 (16.00), 8.965 (0.65).

### Example 72

### 8-bromo-N-{[5-(4-chlorophenyl)-1H-imidazol-2-yl]methyl}-2-(morpholin-4-yl)pyrazolo[1,5-a][1,3,5]triazin-4-amine

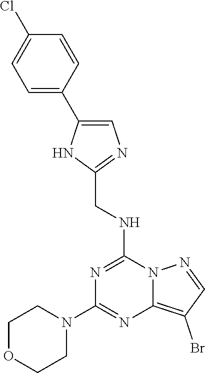 [SMILES: Clc1ccc(-c2cnc(CNc3nc(N4CCOCC4)nc4c(Br)cnn34)[nH]2)cc1]

{[8-bromo-2-(morpholin-4-yl)pyrazolo[1,5-a][1,3,5]triazin-4-yl]amino}ethanimidamide hydrogen chloride (1/1) (Intermediate 48, 100 mg, 255 μmol) and 2-bromo-1-(4-chlorophenyl)ethan-1-one (77.5 mg, 332 μmol) were dissolved in N,N-dimethylformamide (2.5 mL), potassium carbonate (141 mg, 1.02 mmol) was added and the mixture was stirred over night at 90° C. The mixture was filtered and concentrated. The remaining material was purified by preparative HPLC (HT basic) to give 15.5 mg (11% yield) of the title compound.

LC-MS (Method 2): Rₜ=1.21 min; MS (ESIpos): m/z=489 [M+H]⁺

¹H-NMR (400 MHz, DMSO-d6) δ[ppm]: 2.337 (0.54), 2.518 (7.40), 2.523 (4.93), 2.540 (1.62), 2.674 (1.22), 2.679 (0.57), 3.563 (4.93), 3.573 (4.10), 3.670 (5.64), 3.682 (6.55), 3.693 (3.81), 4.700 (9.85), 7.364 (2.70), 7.385 (3.05), 7.565 (0.85), 7.727 (2.14), 7.747 (1.94), 8.007 (16.00), 8.020 (1.31).

### Example 73

### N-[(1H-benzimidazol-2-yl)methyl]-8-bromo-2-[(2R,6S)-2,6-dimethylmorpholin-4-yl]pyrazolo[1,5-a][1,3,5]triazin-4-amine

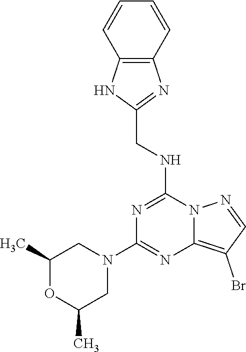 [SMILES: C[C@@H]1CN(c2nc(NCc3nc4ccccc4[nH]3)n3ncc(Br)c3n2)C[C@H](C)O1]

N-[(1H-benzimidazol-2-yl)methyl]-8-bromo-2-(methanesulfonyl)pyrazolo[1,5-a][1,3,5]triazin-4-amine (Intermediate 9, 100 mg, 213 μmol) was suspended in acetonitrile (2.0 mL), (2R,6S)-2,6-dimethylmorpholine (79 μL, 640 μmol) and N-ethyl-N-isopropylpropan-2-amine (110 μL, 640 μmol; CAS-RN:[7087-68-5]) were added and the mixture was stirred for 21 h at 70° C. in a sealed tube. The mixture was diluted with water and extracted with ethyl acetate. The combined organic layers were filtered, dried and concentrated. The remaining material was purified by preparative HPLC (HT basic) to give 54.6 mg (53% yield) of the title compound.

LC-MS (Method 2): Rₜ=1.15 min; MS (ESIpos): m/z=457 [M+H]⁺

¹H-NMR (400 MHz, DMSO-d6) δ[ppm]: 1.011 (1.26), 2.073 (1.99), 2.318 (0.49), 2.322 (0.79), 2.326 (1.00), 2.331 (0.84), 2.336 (0.58), 2.382 (0.58), 2.518 (2.38), 2.522 (1.61), 2.664 (0.54), 2.668 (0.70), 2.673 (0.51), 4.356 (0.44), 4.861 (3.53), 7.115 (2.10), 7.123 (2.24), 7.131 (2.31), 7.138 (2.24), 7.407 (0.47), 7.516 (0.47), 8.030 (16.00), 8.045 (0.47), 9.137 (0.44), 12.283 (0.44).

### Example 74

### 8-bromo-2-(morpholin-4-yl)-N-({5-[2-(trifluoromethoxy)phenyl]-4H-1,2,4-triazol-3-yl}methyl)pyrazolo[1,5-a][1,3,5]triazin-4-amine

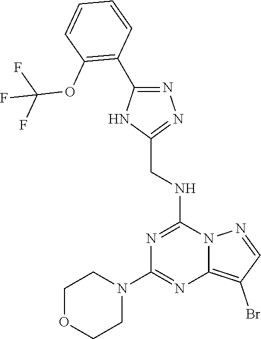 [SMILES: FC(F)(F)Oc1ccccc1-c1nnc(CNc2nc(N3CCOCC3)nc3c(Br)cnn23)[nH]1]

2-{[8-bromo-2-(morpholin-4-yl)pyrazolo[1,5-a][1,3,5]triazin-4-yl]amino}acetohydrazide (Intermediate 39, 100 mg, 269 μmol) and 2-(trifluoromethoxy)benzene-1-carboximidamide hydrogen chloride (1/1) (77.8 mg, 323 μmol) were dissolved DMF (2.5 mL), sodium ethanolate (36.7 mg, 539 μmol) was added and the mixture was stirred for 45 min at 180° C. in the microwave, over night at 90° C. and for 1 hour at 180° C. in the micro wave. The mixture was filtrated and the remaining material was purified by preparative HPLC (HT basic) to give 25.1 mg which were further purified by preparative HPLC to give 6.4 mg (4% yield) of the title compound.

### Preparative HPLC Method

Instrument: Waters Autopurificationsystem; Column: Waters XBrigde C18 5μ 50×50 mm; eluent A: water+0.2 Vol-% aqueous ammonia (32%), eluent B: acetonitrile; gradient: 0.00-0.50 min 20% B (50-100 mL/min), 0.51-8.50 min 20-40% B (100 mL/min), DAD scan: 210-400 nm.

### Analytical HPLC Method

Instrument: Waters Acquity UPLCMS SingleQuad; Column: Acquity UPLC BEH C18 1.7 μm, 50×2.1 mm; eluent A: water+0.2 Vol-% aqueous ammonia (32%), eluent B: acetonitrile; gradient: 0-2.6 min 1-99% B, 2.6-3.0 min 99% B; flow 0.8 mL/min; temperature: 60° C.; DAD scan: 210-400 nm.

Analytical HPLC: Rₜ=1.17 min.

LC-MS (Method 2): Rₜ=0.96 min; MS (ESIpos): m/z=538 [M+H]⁺

¹H-NMR (400 MHz, DMSO-d6) δ[ppm]: 1.233 (0.67), 1.905 (0.67), 2.518 (14.71), 2.523 (9.12), 2.540 (2.08), 3.538 (5.29), 3.637 (6.17), 4.796 (7.46), 7.470 (1.46), 7.489 (3.33), 7.504 (3.12), 7.508 (2.50), 7.523 (2.25), 7.527 (1.83), 7.544 (1.67), 7.548 (1.75), 7.563 (1.67), 7.567 (1.67), 7.582 (0.67), 8.004 (1.58), 8.022 (16.00).

### Example 75

### 8-bromo-N-{[5-(2-fluorophenyl)-4H-1,2,4-triazol-3-yl]methyl}-2-(morpholin-4-yl)pyrazolo[1,5-a][1,3,5]triazin-4-amine

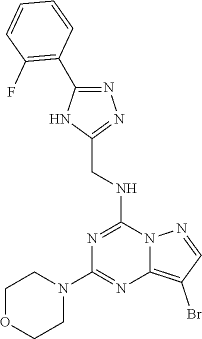 [SMILES: Fc1ccccc1-c1nnc(CNc2nc(N3CCOCC3)nc3c(Br)cnn23)[nH]1]

2-{[8-bromo-2-(morpholin-4-yl)pyrazolo[1,5-a][1,3,5]triazin-4-yl]amino}acetohydrazide (Intermediate 39, 100 mg, 269 μmol) and 2-fluorobenzene-1-carboximidamide hydrogen chloride (1/1) (56.4 mg, 323 μmol) were dissolved in DMF (2.5 mL), sodium ethanolate (36.7 mg, 539 μmol) were added and the mixture was stirred for 45 min at 180° C. in the microwave. The mixture was filtered and purified by preparative HPLC (HT basic) to obtain a product which was treated with DMSO. The precipitate was isolated by filtration to give 7.8 mg (6% yield) of the title compound.

LC-MS (Method 2): Rₜ=0.84 min; MS (ESIpos): m/z=474 [M+H]⁺

¹H-NMR (400 MHz, DMSO-d6) δ[ppm]: 2.074 (1.13), 2.336 (0.47), 2.518 (6.08), 2.523 (4.15), 2.540 (1.41), 2.679 (0.50), 3.549 (4.89), 3.649 (5.31), 3.662 (6.25), 3.672 (3.65), 4.781 (3.95), 7.294 (1.08), 7.312 (2.38), 7.322 (1.02), 7.331 (1.63), 7.343 (1.22), 7.371 (0.94), 7.469 (0.58), 7.484 (0.99), 7.501 (0.91), 7.926 (1.41), 7.931 (1.38), 7.945 (2.68), 7.950 (2.60), 7.965 (1.38), 7.969 (1.24), 8.021 (16.00), 8.034 (0.47), 9.126 (0.64).

### Example 76

### 8-bromo-2-(4-methoxypiperidin-1-yl)-N-[(5-phenyl-4H-1,2,4-triazol-3-yl)methyl]pyrazolo[1,5-a][1,3,5]triazin-4-amine

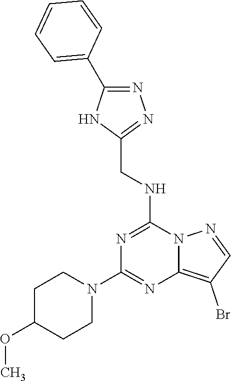 [SMILES: COC1CCN(c2nc(NCc3nnc(-c4ccccc4)[nH]3)n3ncc(Br)c3n2)CC1]

8-bromo-2-(methanesulfonyl)-N-[(5-phenyl-4H-1,2,4-triazol-3-yl)methyl]pyrazolo[1,5-a][1,3,5]triazin-4-amine (Intermediate 24, 100 mg, 200 μmol) was suspended in acetonitrile (2.0 mL), 4-methoxypiperidine (69.2 mg, 601 μmol) and N-ethyl-N-isopropylpropan-2-amine (100 μL, 600 μmol) were added and the mixture was stirred for 5 h at 70° C. in a sealed tube. The mixture was diluted with water and extracted with ethyl acetate. The combined organic layers were concentrated. The remaining material was purified by preparative HPLC (HT acidic) to give 64.1 mg (64% yield) of the title compound.

LC-MS (Method 2): Rₜ=0.97 min; MS (ESIpos): m/z=484 [M+H]⁺

¹H-NMR (400 MHz, DMSO-d6) δ[ppm]: 1.268 (0.37), 1.749 (0.38), 2.518 (1.51), 2.523 (1.07), 2.540 (0.62), 3.205 (16.00), 3.258 (0.40), 3.283 (0.67), 3.352 (0.98), 3.362 (0.56), 3.373 (0.35), 3.378 (0.26), 4.078 (0.46), 4.749 (0.54), 7.449 (0.67), 7.466 (0.72), 7.954 (1.57), 7.971 (1.40), 7.993 (4.28), 8.007 (0.16), 9.051 (0.18).

### Example 77

### methyl 1-(8-bromo-4-{[(5-phenyl-4H-1,2,4-triazol-3-yl)methyl]amino}pyrazolo[1,5-a][1,3,5]triazin-2-yl)piperidine-4-carboxylate

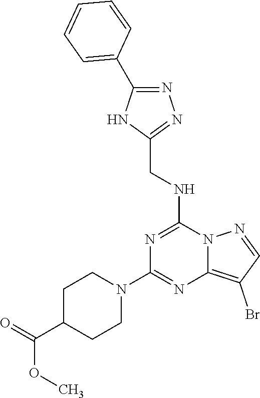 [SMILES: COC(=O)C1CCN(c2nc(NCc3nnc(-c4ccccc4)[nH]3)n3ncc(Br)c3n2)CC1]

8-bromo-2-(methanesulfonyl)-N-[(5-phenyl-4H-1,2,4-triazol-3-yl)methyl]pyrazolo[1,5-a][1,3,5]triazin-4-amine (Intermediate 24 200 mg, 401 μmol) was suspended in acetonitrile (4.0 mL), methyl piperidine-4-carboxylate (172 mg, 1.20 mmol) and N-ethyl-N-isopropylpropan-2-amine (210 μL, 1.2 mmol) were added and the mixture was stirred for 5 h at 70° C. in a sealed tube. The mixture was diluted with water and extracted with ethyl acetate. The combined organic layers were concentrated. The remaining material was purified by preparative HPLC (HT acidic) to 11.0 mg (5% yield) of the title compound.

LC-MS (Method 2): Rₜ=1.00 min; MS (ESIpos): m/z=512 [M+H]⁺

¹H-NMR (400 MHz, DMSO-d6) δ[ppm]: 1.353 (0.52), 1.372 (0.49), 1.766 (0.57), 2.336 (0.24), 2.518 (3.56), 2.523 (2.55), 2.539 (3.56), 2.556 (0.47), 2.567 (0.35), 2.575 (0.47), 2.584 (0.72), 2.594 (0.44), 2.602 (0.27), 2.612 (0.35), 2.678 (0.24), 2.933 (0.66), 2.960 (1.16), 2.989 (0.65), 3.381 (0.17), 3.567 (16.00), 4.441 (0.72), 4.473 (0.70), 4.752 (1.15), 7.446 (1.25), 7.464 (1.32), 7.944 (2.32), 7.961 (2.11), 7.965 (1.73), 8.002 (6.08), 8.016 (0.20), 9.074 (0.30).

### Example 78

### 8-bromo-2-[(2R,6S)-2,6-dimethylmorpholin-4-yl]-N-[(5-phenyl-4H-1,2,4-triazol-3-yl)methyl]pyrazolo[1,5-a][1,3,5]triazin-4-amine

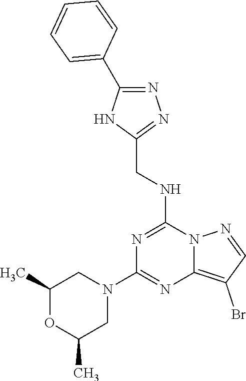 [SMILES: C[C@@H]1CN(c2nc(NCc3nnc(-c4ccccc4)[nH]3)n3ncc(Br)c3n2)C[C@H](C)O1]

8-bromo-2-(methanesulfonyl)-N-[(5-phenyl-4H-1,2,4-triazol-3-yl)methyl]pyrazolo[1,5-a][1,3,5]triazin-4-amine (Intermediate 24, 100 mg, 200 μmol) was suspended in acetonitrile (2.0 mL), (2R,6S)-2,6-dimethylmorpholine (74 μL, 600 μmol) and N-ethyl-N-isopropylpropan-2-amine (100 μL, 600 μmol) were added and the mixture was stirred for 5 h at 70° C. in a sealed tube. The mixture was diluted with water and extracted with ethyl acetate. The combined organic layers were concentrated. The remaining material was purified by preparative HPLC (HT acidic) to 31.5 mg (31% yield) of the title compound.

LC-MS (Method 2): Rₜ=1.00 min; MS (ESIneg): m/z=482 [M−H]⁻

¹H-NMR (400 MHz, DMSO-d6) δ[ppm]: 1.061 (4.30), 2.331 (1.39), 2.336 (0.66), 2.518 (6.45), 2.522 (4.43), 2.539 (9.99), 2.669 (1.87), 2.673 (1.33), 2.678 (0.57), 3.410 (1.20), 4.403 (1.36), 4.766 (2.56), 7.447 (2.85), 7.464 (2.94), 7.954 (6.20), 7.971 (5.50), 7.974 (4.49), 8.017 (16.00), 8.031 (0.70), 9.112 (0.63).

### Example 79

### 8-bromo-N-{[5-(3-fluorophenyl)-1H-imidazol-2-yl]methyl}-2-(morpholin-4-yl)pyrazolo[1,5-a][1,3,5]triazin-4-amine

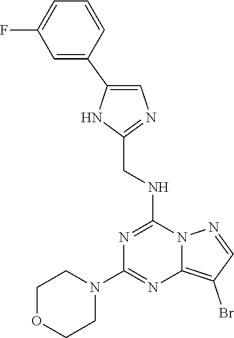 [SMILES: Fc1cccc(-c2cnc(CNc3nc(N4CCOCC4)nc4c(Br)cnn34)[nH]2)c1]

{[8-bromo-2-(morpholin-4-yl)pyrazolo[1,5-a][1,3,5]triazin-4-yl]amino}ethanimidamide hydrogen chloride (1/1) (Intermediate 48, 150 mg, 383 μmol) and 2-bromo-1-(3-fluorophenyl)ethan-1-one (108 mg, 498 μmol) were dissolved in N,N-dimethylformamide (2.5 mL), cesium carbonate (374 mg, 1.15 mmol) was added and the mixture was stirred for 3 h at room temperature and for 4 h at 60° C. The mixture was filtered and purified by preparative HPLC (1. HT basic) to give 4.6 mg (2% yield) of the title compound. An impure fraction of the first HPLC purification was further purified by preparative HPLC to give 2.5 mg (1% yield) of the title compound.

### Preparative HPLC Method

Instrument: Waters Autopurificationsystem; Column: Waters XBrigde C18 5μ 50×50 mm; eluent A: water+0.2 Vol-% aqueous ammonia (32%), eluent B: acetonitrile; gradient: 0.00-0.50 min 25% B (50-100 mL/min), 0.51-8.50 min 25-45% B (100 mL/min), DAD scan: 210-400 nm.

### Analytical HPLC Method

Instrument: Waters Acquity UPLCMS SingleQuad; Column: Acquity UPLC BEH C18 1.7 μm, 50×2.1 mm; eluent A: water+0.2 Vol-% aqueous ammonia (32%), eluent B: acetonitrile; gradient: 0-2.6 min 1-99% B, 2.6-3.0 min 99% B; flow 0.8 mL/min; temperature: 60° C.; DAD scan: 210-400 nm.

Analytical HPLC: Rₜ=1.20 min.

LC-MS (Method 2): Rₜ=1.14 min; MS (ESIpos): m/z=473 [M+H]⁺

¹H-NMR (400 MHz, DMSO-d6) δ[ppm]: 1.233 (0.79), 1.907 (0.79), 2.323 (2.41), 2.327 (3.42), 2.332 (2.47), 2.336 (1.12), 2.518 (16.00), 2.523 (10.22), 2.540 (2.75), 2.665 (2.47), 2.669 (3.42), 2.673 (2.47), 3.563 (5.89), 3.670 (6.74), 3.683 (7.92), 3.693 (4.60), 4.700 (5.45), 4.714 (5.45), 6.974 (1.18), 7.350 (1.18), 7.369 (1.18), 7.490 (1.74), 7.517 (1.80), 7.561 (1.40), 7.579 (1.18), 7.623 (1.46), 8.010 (14.88), 8.993 (1.12), 9.006 (2.19), 9.021 (1.07), 12.031 (0.73).

### Example 80

### 8-bromo-N-{[5-(3-methoxyphenyl)-1H-imidazol-2-yl]methyl}-2-(morpholin-4-yl)pyrazolo[1,5-a][1,3,5]triazin-4-amine

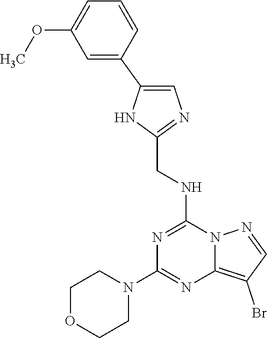 [SMILES: COc1cccc(-c2cnc(CNc3nc(N4CCOCC4)nc4c(Br)cnn34)[nH]2)c1]

{[8-bromo-2-(morpholin-4-yl)pyrazolo[1,5-a][1,3,5]triazin-4-yl]amino}ethanimidamide hydrogen chloride (1/1) (Intermediate 48, 150 mg, 383 μmol) and 2-bromo-1-(3-methoxyphenyl)ethan-1-one (114 mg, 498 μmol) were dissolved in N,N-dimethylformamide (2.5 mL), cesium carbonate (374 mg, 1.15 mmol) was added and the mixture was stirred for 3 h at room temperature and for 4 h at 60° C. The mixture was filtered and purified by preparative HPLC twice (1. HT basic, 2. see below) to give 4.3 mg (2% yield) of the title compound.

### Preparative HPLC Method

Instrument: Waters Autopurificationsystem; Column: Waters XBrigde C18 5μ50×50 mm; eluent A: water+0.2 Vol-% aqueous ammonia (32%), eluent B: acetonitrile; gradient: 0.00-0.50 min 25% B (50-100 mL/min), 0.51-8.50 min 25-45% B (100 mL/min), DAD scan: 210-400 nm.

### Analytical HPLC Method

Instrument: Waters Acquity UPLCMS SingleQuad; Column: Acquity UPLC BEH C18 1.7 μm, 50×2.1 mm; eluent A: water+0.2 Vol-% aqueous ammonia (32%), eluent B: acetonitrile; gradient: 0-2.6 min 1-99% B, 2.6-3.0 min 99% B; flow 0.8 mL/min; temperature: 60° C.; DAD scan: 210-400 nm.

Analytical HPLC: Rₜ=1.09 min.

LC-MS (Method 2): Rₜ=1.11 min; MS (ESIpos): m/z=485 [M+H]⁺

¹H-NMR (400 MHz, DMSO-d6) δ[ppm]: 1.229 (0.46), 1.903 (0.38), 2.323 (1.67), 2.665 (2.48), 3.561 (4.56), 3.669 (4.91), 3.681 (5.79), 3.765 (16.00), 4.697 (3.36), 4.711 (3.39), 6.730 (0.87), 6.746 (0.98), 7.231 (1.23), 7.252 (1.15), 7.278 (2.32), 7.520 (0.71), 8.005 (6.03), 8.992 (1.37), 11.959 (0.27).

### Example 81

### 8-bromo-N-{[5-(3-chlorophenyl)-1H-imidazol-2-yl]methyl}-2-(morpholin-4-yl)pyrazolo[1,5-a][1,3,5]triazin-4-amine

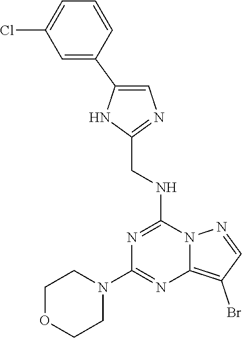 [SMILES: Clc1cccc(-c2cnc(CNc3nc(N4CCOCC4)nc4c(Br)cnn34)[nH]2)c1]

{[8-bromo-2-(morpholin-4-yl)pyrazolo[1,5-a][1,3,5]triazin-4-yl]amino}ethanimidamide hydrogen chloride (1/1) (Intermediate 48, 150 mg, 383 μmol) and 2-bromo-1-(3-chlorophenyl)ethan-1-one (116 mg, 498 μmol) were dissolved in N,N-dimethylformamide (2.5 mL), cesium carbonate (374 mg, 1.15 mmol) was added and the mixture was stirred over night at 90° C. in the microwave. The mixture was filtered and purified by preparative HPLC (HT basic) to give 3.9 mg (2% yield) of the title compound. An impure fraction of the first HPLC purification was further purified by preparative HPLC to give 3.9 mg (2% yield) of the title compound.

### Preparative HPLC Method

Instrument: Waters Autopurificationsystem; Column: Waters XBrigde C18 5μ 50×50 mm; eluent A: water+0.2 Vol-% aqueous ammonia (32%), eluent B: acetonitrile; gradient: 0.00-0.50 min 35% B (50-100 mL/min), 0.51-8.50 min 35-55% B (100 mL/min), DAD scan: 210-400 nm.

### Analytical HPLC Method

Instrument: Waters Acquity UPLCMS SingleQuad; Column: Acquity UPLC BEH C18 1.7 μm, 50×2.1 mm; eluent A: water+0.2 Vol-% aqueous ammonia (32%), eluent B: acetonitrile; gradient: 0-2.6 min 1-99% B, 2.6-3.0 min 99% B; flow 0.8 mL/min; temperature: 60° C.; DAD scan: 210-400 nm.

Analytical HPLC: Rₜ=1.20 min.

LC-MS (Method 2): Rₜ=1.22 min; MS (ESIpos): m/z=489 [M+H]⁺

¹H-NMR (400 MHz, DMSO-d6) δ[ppm]: 2.326 (1.64), 2.331 (1.17), 2.336 (0.52), 2.518 (5.67), 2.522 (3.93), 2.668 (1.69), 2.673 (1.17), 2.678 (0.55), 3.566 (5.15), 3.672 (5.89), 3.684 (6.51), 3.695 (3.65), 4.702 (6.87), 7.189 (1.39), 7.191 (1.36), 7.208 (1.74), 7.212 (1.80), 7.325 (1.88), 7.345 (3.24), 7.365 (1.66), 7.652 (5.91), 7.685 (2.15), 7.704 (2.02), 7.768 (2.48), 7.772 (3.38), 8.009 (16.00), 8.995 (0.46), 12.048 (0.49).

### Example 82

### 8-bromo-2-(morpholin-4-yl)-N-({5-[3-(trifluoromethoxy)phenyl]-1H-imidazol-2-yl}methyl)pyrazolo[1,5-a][1,3,5]triazin-4-amine

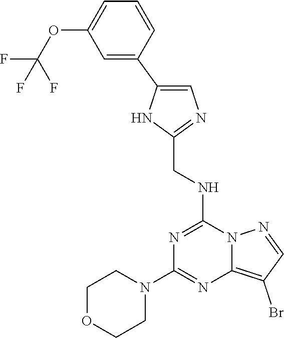 [SMILES: FC(F)(F)Oc1cccc(-c2cnc(CNc3nc(N4CCOCC4)nc4c(Br)cnn34)[nH]2)c1]

{[8-bromo-2-(morpholin-4-yl)pyrazolo[1,5-a][1,3,5]triazin-4-yl]amino}ethanimidamide hydrogen chloride (1/1) (Intermediate 48, 150 mg, 383 μmol) and 2-bromo-1-[3-(trifluoromethoxy)phenyl]ethan-1-one (141 mg, 498 μmol) were dissolved in N,N-dimethylformamide (2.5 mL), cesium carbonate (374 mg, 1.15 mmol) was added and the mixture was stirred over night at 90° C. in the microwave. The mixture was filtered and purified by preparative HPLC (HT basic) to give 2.8 mg (1% yield) of the title compound. An impure fraction of the first purification by HPLC was further purified by preparative HPLC to give 2.6 mg (1% yield) of the title compound.

### Preparative HPLC Method

Instrument: Waters Autopurificationsystem; Column: Waters XBrigde C18 5μ 50×50 mm; eluent A: water+0.2 Vol-% aqueous ammonia (32%), eluent B: acetonitrile; gradient: 0.00-0.50 min 18% B (50-100 mL/min), 0.51-8.50 min 35-55% B (100 mL/min), DAD scan: 210-400 nm.

### Analytical HPLC Method

Instrument: Waters Acquity UPLCMS SingleQuad; Column: Acquity UPLC BEH C18 1.7 μm, 50×2.1 mm; eluent A: water+0.2 Vol-% aqueous ammonia (32%), eluent B: acetonitrile; gradient: 0-2.6 min 1-99% B, 2.6-3.0 min 99% B; flow 0.8 mL/min; temperature: 60° C.; DAD scan: 210-400 nm.

Analytical HPLC: Rₜ=1.25 min.

LC-MS (Method 2): Rₜ=1.29 min; MS (ESIpos): m/z=539 [M+H]⁺

¹H-NMR (400 MHz, DMSO-d6) δ[ppm]: 2.326 (2.64), 2.331 (1.87), 2.336 (0.85), 2.518 (9.06), 2.522 (6.26), 2.668 (2.64), 2.673 (1.91), 2.678 (0.81), 3.559 (5.70), 3.668 (7.23), 3.681 (7.96), 3.692 (4.51), 3.885 (0.60), 4.703 (4.13), 7.123 (1.70), 7.143 (1.91), 7.415 (0.51), 7.428 (2.98), 7.448 (5.32), 7.468 (2.89), 7.682 (8.26), 7.748 (3.02), 7.768 (2.68), 8.009 (16.00), 9.023 (1.36), 12.071 (1.74).

### Example 83

### 8-bromo-2-(morpholin-4-yl)-N-({5-[3-(trifluoromethyl)phenyl]-1H-imidazol-2-yl}methyl)pyrazolo[1,5-a][1,3,5]triazin-4-amine

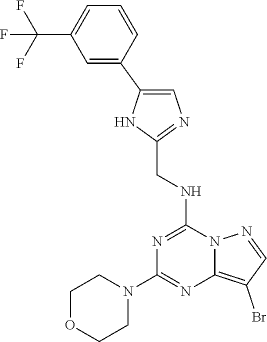 [SMILES: FC(F)(F)c1cccc(-c2cnc(CNc3nc(N4CCOCC4)nc4c(Br)cnn34)[nH]2)c1]

{[8-bromo-2-(morpholin-4-yl)pyrazolo[1,5-a][1,3,5]triazin-4-yl]amino}ethanimidamide hydrogen chloride (1/1) (Intermediate 48, 150 mg, 383 μmol) and 2-bromo-1-[3-(trifluoromethyl)phenyl]ethan-1-one (133 mg, 498 μmol) were dissolved in N,N-dimethylformamide (2.5 mL), cesium carbonate (374 mg, 1.15 mmol) was added and the mixture was stirred over night at 90° C. in the microwave. The mixture was filtered and purified by preparative HPLC (HT basic) to give 13.4 mg (6% yield) of the title compound.

LC-MS (Method 2): Rₜ=1.26 min; MS (ESIpos): m/z=523 [M+H]⁺

¹H-NMR (400 MHz, DMSO-d6) δ[ppm]: 2.336 (0.57), 2.518 (7.61), 2.523 (5.27), 2.678 (0.57), 3.561 (4.88), 3.671 (6.11), 3.684 (6.80), 3.694 (3.90), 4.717 (3.51), 7.491 (1.32), 7.511 (2.55), 7.540 (2.01), 7.559 (2.79), 7.578 (1.17), 7.742 (5.15), 8.011 (16.00), 8.036 (2.01), 8.055 (3.57), 9.030 (1.05), 12.098 (1.29).

### Example 84

### 8-bromo-2-(morpholin-4-yl)-N-{[5-(pyridin-3-yl)-1H-imidazol-2-yl]methyl}pyrazolo[1,5-a][1,3,5]triazin-4-amine

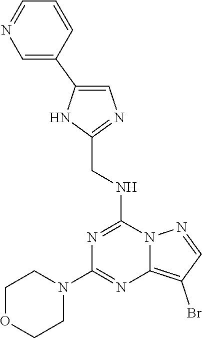 [SMILES: Brc1cnn2c(NCc3ncc(-c4cccnc4)[nH]3)nc(N3CCOCC3)nc12]

{[8-bromo-2-(morpholin-4-yl)pyrazolo[1,5-a][1,3,5]triazin-4-yl]amino}ethanimidamide hydrogen chloride (1/1) (Intermediate 48, 150 mg, 383 μmol) and 2-bromo-1-(pyridin-3-yl)ethan-1-one hydrogen bromide (1/1) (140 mg, 498 μmol) were dissolved in N,N-dimethylformamide (2.5 mL), cesium carbonate (374 mg, 1.15 mmol) was added and the mixture was stirred over night at 90° C. in a sealed tube. Cesium carbonate (130 mg, 400 μmol) was added and the mixture was stirred for 3 days at 60° C. The mixture was filtered and purified by preparative HPLC to give 6.3 mg (3% yield) of the title compound.

### Preparative HPLC Method

Instrument: Waters Autopurificationsystem; Column: XBrigde C18 5μ, 100×30 mm; eluent A: water+0.2 vol % aqueous ammonia (32%); eluent B: methanol; gradient: 0.0-0.5 min 45% B (35-70 mL/min), 0.5-5.5 min 45-60% B; flow: 70 mL/min; temperature: 25° C.; DAD scan: 210-400 nm.

### Analytical HPLC Method

Instrument: Waters Acquity UPLCMS SingleQuad; Column: Acquity UPLC BEH C18 1.7μ, 50×2.1 mm; eluent A: water+0.2 vol % aqueous ammonia (32%); eluent B: methanol; gradient: 0-1.6 min 1-99% B, 1.6-2.0 min 99% B; flow: 0.7 mL/min; temperature: 60° C.; DAD scan: 210-400 nm.

Analytical HPLC: Rₜ=1.23 min.

LC-MS (Method 2): Rₜ=1.91 min; MS (ESIpos): m/z=456 [M+H]⁺

¹H-NMR (400 MHz, DMSO-d6) δ[ppm]: 0.831 (0.91), 0.851 (0.91), 1.136 (1.68), 1.232 (5.49), 1.351 (1.07), 1.422 (0.61), 1.906 (0.91), 2.114 (0.76), 2.178 (0.46), 2.326 (4.42), 2.331 (3.20), 2.518 (16.00), 2.522 (10.97), 2.539 (1.52), 2.668 (4.42), 2.673 (3.20), 3.563 (3.81), 3.670 (4.42), 3.683 (5.03), 4.712 (3.50), 4.727 (3.50), 7.329 (0.76), 7.348 (0.91), 7.360 (0.91), 7.672 (1.98), 8.011 (9.30), 8.025 (0.76), 8.047 (1.07), 8.067 (1.07), 8.359 (1.37), 8.368 (1.37), 8.950 (1.83), 9.023 (1.22), 12.081 (1.07).

### Example 85

### 8-bromo-N-{[5-(2-methylphenyl)-1H-imidazol-2-yl]methyl}-2-(morpholin-4-yl)pyrazolo[1,5-a][1,3,5]triazin-4-amine

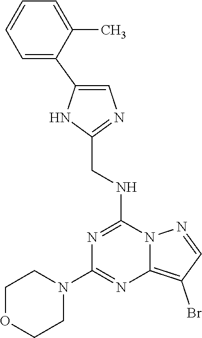 [SMILES: Cc1ccccc1-c1cnc(CNc2nc(N3CCOCC3)nc3c(Br)cnn23)[nH]1]

{[8-bromo-2-(morpholin-4-yl)pyrazolo[1,5-a][1,3,5]triazin-4-yl]amino}ethanimidamide hydrogen chloride (1/1) (Intermediate 48, 150 mg, 383 μmol) and 2-bromo-1-(2-methylphenyl)ethan-1-one (106 mg, 498 μmol) were dissolved in N,N-dimethylformamide (2.5 mL), cesium carbonate (374 mg, 1.15 mmol) was added and the mixture was stirred over night at 90° C. in a sealed tube. The mixture was filtered and purified by preparative HPLC twice (1. HT basic, 2. see below) to give 15.6 mg (9% yield) of the title compound.

### Preparative HPLC Method

Instrument: Waters Autopurificationsystem; Column: Waters XBrigde C18 5μ 50×50 mm; eluent A: water+0.2 vol % aqueous ammonia (32%), eluent B: acetonitrile; gradient: 0.00-0.50 min 27% B (50-100 mL/min), 0.51-8.50 min 27-47% B (100 mL/min), DAD scan: 210-400 nm.

### Analytical HPLC Method

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-2.6 min 1-99% B, 2.6-3.0 min 99% B; flow 0.8 mL/min; temperature: 60° C.; DAD scan: 210-400 nm.

Analytical HPLC: Rₜ=1.15 min.

LC-MS (Method 2): Rₜ=1.16 min; MS (ESIpos): m/z=469 [M+H]⁺

¹H-NMR (400 MHz, DMSO-d6) δ[ppm]: 1.232 (0.52), 2.331 (1.06), 2.336 (0.61), 2.350 (3.88), 2.413 (16.00), 2.449 (0.70), 2.518 (4.94), 2.522 (3.34), 2.539 (0.74), 2.673 (0.97), 3.574 (6.51), 3.585 (5.23), 3.681 (6.60), 3.693 (7.28), 4.725 (4.08), 6.969 (1.06), 7.078 (0.70), 7.082 (0.74), 7.096 (2.03), 7.115 (1.71), 7.119 (1.62), 7.157 (1.44), 7.177 (4.46), 7.195 (2.84), 7.210 (0.61), 7.226 (0.50), 7.243 (4.24), 7.262 (0.86), 7.349 (0.65), 7.367 (0.45), 7.767 (2.28), 7.787 (1.87), 7.999 (1.98), 8.008 (9.83), 8.979 (1.06), 11.972 (1.37), 12.082 (0.52).

### Example 86

### 8-bromo-N-{[5-(2-fluorophenyl)-1H-imidazol-2-yl]methyl}-2-(morpholin-4-yl)pyrazolo[1,5-a][1,3,5]triazin-4-amine

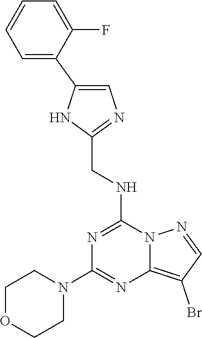 [SMILES: Fc1ccccc1-c1cnc(CNc2nc(N3CCOCC3)nc3c(Br)cnn23)[nH]1]

{[8-bromo-2-(morpholin-4-yl)pyrazolo[1,5-a][1,3,5]triazin-4-yl]amino}ethanimidamide hydrogen chloride (1/1) (Intermediate 48, 150 mg, 383 μmol) and 2-bromo-1-(2-fluorophenyl)ethan-1-one (108 mg, 498 μmol) were dissolved in N,N-dimethylformamide (2.5 mL), cesium carbonate (374 mg, 1.15 mmol) was added and the mixture was stirred over night at 90° C. in a sealed tube. The mixture was filtered and purified by preparative HPLC twice (1. HT basic, 2. see below) to give 18.7 mg (10% yield) of the title compound.

### Preparative HPLC Method

Instrument: Waters Autopurificationsystem; Column: Waters XBrigde C18 5μ 50×50 mm; eluent A: water+0.2 vol % aqueous ammonia (32%), eluent B: acetonitrile; gradient: 0.00-0.50 min 27% B (50-100 mL/min), 0.51-8.50 min 27-47% B (100 mL/min), DAD scan: 210-400 nm.

### Analytical HPLC Method

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-2.6 min 1-99% B, 2.6-3.0 min 99% B; flow 0.8 mL/min; temperature: 60° C.; DAD scan: 210-400 nm.

Analytical HPLC: Rₜ=1.14 min.

LC-MS (Method 2): Rₜ=1.15 min; MS (ESIpos): m/z=473 [M+H]⁺

¹H-NMR (400 MHz, DMSO-d6) δ[ppm]: 1.108 (16.00), 1.231 (0.47), 2.074 (0.77), 2.327 (1.71), 2.331 (1.19), 2.337 (0.55), 2.518 (6.32), 2.523 (4.30), 2.669 (1.68), 2.674 (1.19), 2.678 (0.55), 3.563 (4.03), 3.673 (4.72), 3.685 (5.52), 3.696 (3.17), 4.191 (1.05), 4.734 (4.94), 7.197 (2.40), 7.219 (3.09), 7.370 (1.66), 7.380 (1.54), 7.982 (0.50), 7.991 (0.69), 8.013 (12.36), 9.011 (0.44), 12.102 (0.50).

### Example 87

### 8-bromo-N-{[5-(2-methoxyphenyl)-1H-imidazol-2-yl]methyl}-2-(morpholin-4-yl)pyrazolo[1,5-a][1,3,5]triazin-4-amine

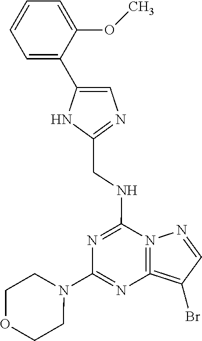

{[8-bromo-2-(morpholin-4-yl)pyrazolo[1,5-a][1,3,5]triazin-4-yl]amino}ethanimidamide hydrogen chloride (1/1) (Intermediate 48, 150 mg, 383 μmol) and 2-bromo-1-(2-methoxyphenyl)ethan-1-one (114 mg, 498 μmol) were dissolved in N,N-dimethylformamide (2.5 mL), cesium carbonate (374 mg, 1.15 mmol) was added and the mixture was stirred over night at 90° C. in a sealed tube. The mixture was filtered and purified by preparative HPLC twice (1. HT basic, 2. see below) to give 6.3 mg (3% yield) of the title compound.

### Preparative HPLC Method

Instrument: Waters Autopurificationsystem; Column: Waters XBrigde C18 5μ 50×50 mm; eluent A: water+0.2 vol % aqueous ammonia (32%), eluent B: acetonitrile; gradient: 0.00-0.50 min 27% B (50-100 mL/min), 0.51-8.50 min 27-47% B (100 mL/min), DAD scan: 210-400 nm.

### Analytical HPLC Method

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-2.6 min 1-99% B, 2.6-3.0 min 99% B; flow 0.8 mL/min; temperature: 60° C.; DAD scan: 210-400 nm.

Analytical HPLC: Rₜ=1.13 min.

LC-MS (Method 2): Rₜ=1.14 min; MS (ESIpos): m/z=485 [M+H]⁺

¹H-NMR (400 MHz, DMSO-d6) δ[ppm]: 1.233 (0.72), 2.327 (1.66), 2.331 (1.19), 2.518 (6.42), 2.523 (4.35), 2.540 (0.72), 2.669 (1.69), 2.673 (1.19), 3.567 (4.01), 3.680 (3.96), 3.692 (5.01), 3.877 (16.00), 4.720 (3.21), 6.927 (0.83), 6.929 (0.89), 6.947 (1.74), 6.964 (1.05), 6.966 (1.11), 7.008 (1.69), 7.027 (2.02), 7.134 (1.08), 7.139 (1.11), 7.157 (1.25), 7.173 (0.69), 7.177 (0.66), 7.275 (0.58), 7.436 (2.74), 8.011 (10.21), 8.016 (1.97), 8.030 (1.66), 8.035 (1.55), 8.981 (0.64), 11.861 (1.00).

### Example 88

### 8-bromo-2-(morpholin-4-yl)-N-({5-[2-(trifluoromethyl)phenyl]-1H-imidazol-2-yl}methyl)pyrazolo[1,5-a][1,3,5]triazin-4-amine

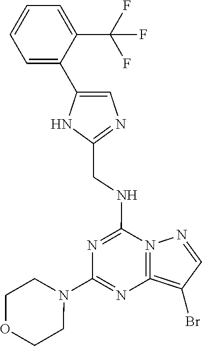

{[8-bromo-2-(morpholin-4-yl)pyrazolo[1,5-a][1,3,5]triazin-4-yl]amino}ethanimidamide hydrogen chloride (1/1) (Intermediate 48, 150 mg, 383 μmol) and 2-bromo-1-[2-(trifluoromethyl)phenyl]ethan-1-one (133 mg, 498 μmol) were dissolved in N,N-dimethylformamide (2.5 mL), cesium carbonate (374 mg, 1.15 mmol) was added and the mixture was stirred over night at 90° C. in a sealed tube. The mixture was filtered and purified by preparative HPLC (HT basic) to give 3.1 mg (1% yield) of the title compound. An impure fraction of the first purification by HPLC was further purified by preparative HPLC to give 5.2 mg (3% yield) of the title compound.

### Preparative HPLC Method

Instrument: Waters Autopurificationsystem; Column: Waters XBrigde C18 5μ 50×50 mm; eluent A: water+0.2 vol % aqueous ammonia (32%), eluent B: acetonitrile; gradient: 0.00-0.50 min 34% B (50-100 mL/min), 0.51-8.50 min 34-54% B (100 mL/min), DAD scan: 210-400 nm.

### Analytical HPLC Method

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-2.6 min 1-99% B, 2.6-3.0 min 99% B; flow 0.8 mL/min; temperature: 60° C.; DAD scan: 210-400 nm.

Analytical HPLC: Rₜ=1.25 min.

LC-MS (Method 2): Rₜ=1.26 min; MS (ESIpos): m/z=523 [M+H]⁺

¹H-NMR (400 MHz, DMSO-d6) δ[ppm]: 2.322 (1.78), 2.326 (2.42), 2.331 (1.70), 2.336 (0.75), 2.518 (7.96), 2.522 (5.39), 2.664 (1.82), 2.668 (2.46), 2.673 (1.78), 3.561 (4.71), 3.670 (5.78), 3.683 (6.42), 3.693 (3.60), 4.717 (5.54), 7.491 (1.23), 7.511 (2.34), 7.540 (1.82), 7.559 (2.53), 7.578 (1.03), 7.742 (5.94), 8.011 (16.00), 8.035 (1.98), 8.055 (3.29), 9.022 (0.55), 12.099 (0.91).

### Example 89

### 8-bromo-2-(morpholin-4-yl)-N-({5-[2-(trifluoromethoxy)phenyl]-1H-imidazol-2-yl}methyl)pyrazolo[1,5-a][1,3,5]triazin-4-amine

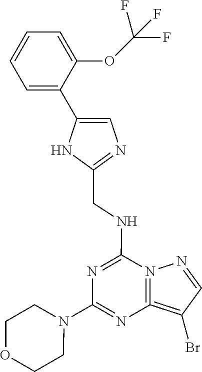

{[8-bromo-2-(morpholin-4-yl)pyrazolo[1,5-a][1,3,5]triazin-4-yl]amino}ethanimidamide hydrogen chloride (1/1) (Intermediate 48, 150 mg, 383 μmol) and 2-bromo-1-[2-(trifluoromethoxy)phenyl]ethan-1-one (141 mg, 498 μmol) were dissolved in N,N-dimethylformamide (2.5 mL), cesium carbonate (374 mg, 1.15 mmol) was added and the mixture was stirred over night at 90° C. in a sealed tube. The mixture was filtered and purified by preparative HPLC (HT basic) to give 50.8 mg (1% yield) of the title compound.

LC-MS (Method 2): Rₜ=1.27 min; MS (ESIpos): m/z=539 [M+H]⁺

¹H-NMR (400 MHz, DMSO-d6) δ[ppm]: 2.331 (1.43), 2.518 (6.85), 2.522 (4.61), 2.669 (1.98), 2.673 (1.39), 3.555 (6.63), 3.667 (7.73), 3.679 (8.75), 3.690 (4.98), 4.734 (7.32), 7.285 (0.84), 7.289 (0.92), 7.306 (2.38), 7.323 (3.00), 7.332 (8.16), 7.350 (3.26), 7.377 (2.67), 7.396 (2.97), 7.414 (1.46), 7.417 (1.32), 8.014 (16.00), 8.028 (2.09), 8.115 (2.71), 8.119 (2.78), 8.134 (2.64), 8.139 (2.45), 9.029 (0.95), 12.109 (1.10).

### Example 90

### 8-bromo-2-(morpholin-4-yl)-N-{[5-(pyridin-2-yl)-1H-imidazol-2-yl]methyl}pyrazolo[1,5-a][1,3,5]triazin-4-amine

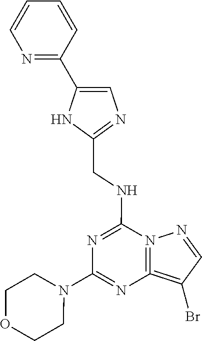

{[8-bromo-2-(morpholin-4-yl)pyrazolo[1,5-a][1,3,5]triazin-4-yl]amino}ethanimidamide hydrogen chloride (1/1) (Intermediate 48, 150 mg, 383 μmol) and 2-bromo-1-(pyridin-2-yl)ethan-1-one hydrogen bromide (1/1) (140 mg, 498 μmol) were dissolved in N,N-dimethylformamide (2.5 mL), cesium carbonate (374 mg, 1.15 mmol) was added and the mixture was stirred over night at 90° C. in a sealed tube. The mixture was filtered and purified by preparative HPLC to give 6.2 mg (3% yield) of the title compound.

### Preparative HPLC Method

Instrument: Waters Autopurificationsystem; Column: Waters XBrigde C18 5μ 50×50 mm; eluent A: water+0.2 vol % aqueous ammonia (32%), eluent B: acetonitrile; gradient: 0.0-0.5 min 12% B (35-70 mL/min), 0.5-5.5 min 24-39% B; flow: 70 mL/min; temperature: 25° C.; DAD scan: 210-400 nm.

### Analytical HPLC Method

Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-2.6 min 1-99% B, 2.6-3.0 min 99% B; flow 0.8 mL/min; temperature: 60° C.; DAD scan: 210-400 nm.

Analytical HPLC: Rₜ=0.94 min.

LC-MS (Method 2): Rₜ=0.97 min; MS (ESIpos): m/z=456 [M+H]⁺

¹H-NMR (400 MHz, DMSO-d6) δ[ppm]: −0.149 (0.50), −0.009 (4.85), 0.007 (3.70), 0.146 (0.50), 1.139 (1.10), 1.235 (1.15), 1.909 (0.70), 2.117 (0.45), 2.324 (2.20), 2.329 (2.90), 2.334 (2.05), 2.520 (10.60), 2.525 (6.90), 2.542 (4.30), 2.666 (2.20), 2.671 (2.95), 2.676 (2.10), 3.563 (6.05), 3.631 (0.60), 3.676 (7.85), 3.689 (8.95), 3.700 (5.40), 4.720 (5.85), 4.734 (5.70), 6.994 (0.50), 7.122 (0.55), 7.167 (1.20), 7.245 (0.55), 7.589 (1.30), 7.690 (0.45), 7.781 (1.95), 7.832 (0.45), 7.960 (0.45), 8.012 (16.00), 8.064 (0.40), 8.136 (0.50), 8.469 (1.15), 8.493 (0.85), 9.010 (0.85), 12.117 (0.65).

### Example 91

### N-[(1H-benzimidazol-2-yl)methyl]-8-(3-fluorophenyl)-2-(morpholin-4-yl)pyrazolo[1,5-a][1,3,5]triazin-4-amine

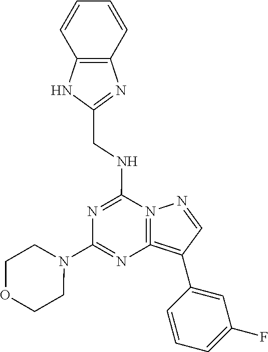

N-[(1H-benzimidazol-2-yl)methyl]-8-(3-fluorophenyl)-2-(methanesulfonyl)pyrazolo[1,5-a][1,3,5]triazin-4-amine (Intermediate 52, 39.0 mg) was provided in acetonitrile (2.0 mL), morpholine (77 μL, 890 μmol) was added and the mixture was stirred for 1 h at 70° C. The mixture was concentrated. The remaining material was purified by flash chromatography using silica gel (amino-phase, dichloromethane/ethyl acetate gradient) to give 8.0 mg of the title compound.

LC-MS (Method 2): Rₜ=1.22 min; MS (ESIpos): m/z=445 [M+H]⁺

¹H-NMR (400 MHz, DMSO-d6) δ[ppm]: 0.833 (0.43), 0.851 (0.82), 1.172 (0.69), 1.232 (3.17), 1.907 (0.69), 1.987 (0.65), 2.287 (1.26), 2.331 (1.86), 2.336 (0.82), 2.518 (9.37), 2.522 (6.20), 2.673 (1.91), 2.678 (0.87), 2.950 (0.65), 2.962 (0.82), 2.974 (0.69), 3.544 (3.08), 3.659 (2.43), 3.683 (5.07), 3.705 (2.60), 3.789 (0.56), 4.894 (3.90), 4.904 (3.77), 5.759 (4.47), 6.905 (0.91), 6.908 (0.95), 6.912 (1.08), 6.914 (1.04), 6.928 (1.86), 6.934 (1.99), 6.948 (1.04), 6.952 (1.17), 6.957 (1.08), 7.105 (0.56), 7.118 (2.30), 7.128 (2.95), 7.134 (2.91), 7.141 (3.21), 7.151 (2.30), 7.164 (0.61), 7.368 (1.34), 7.384 (1.69), 7.388 (2.69), 7.405 (3.90), 7.425 (2.25), 7.530 (1.69), 7.550 (1.52), 7.848 (1.34), 7.852 (1.99), 7.860 (2.91), 7.863 (3.25), 7.866 (2.21), 7.880 (4.47), 7.885 (4.64), 8.052 (0.43), 8.557 (16.00), 9.123 (1.21), 12.283 (2.25).

### Example 92

### N-[(1H-benzimidazol-2-yl)methyl]-8-cyclopropyl-2-(morpholin-4-yl)pyrazolo[1,5-a][1,3,5]triazin-4-amine

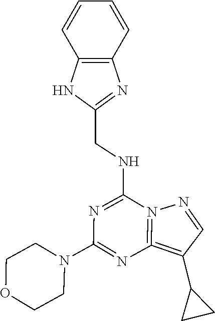

N-[(1H-benzimidazol-2-yl)methyl]-8-cyclopropyl-2-(methanesulfonyl)pyrazolo[1,5-a][1,3,5]triazin-4-amine (Intermediate 57, 90.0 mg, 235 μmol) was provided in acetonitrile (2.0 mL), morpholine (200 μL, 2.3 mmol) was added and the mixture was stirred for 19 h at 70° C. The mixture was concentrated. The remaining material was purified by flash chromatography using silica gel (amino-phase, dichloromethane/ethyl acetate gradient) to give 22.0 mg (24% yield) of the title compound.

LC-MS (Method 2): Rₜ=1.07 min; MS (ESIpos): m/z=391 [M+H]⁺

¹H-NMR (400 MHz, DMSO-d6) δ[ppm]: 0.709 (1.70), 0.721 (5.01), 0.726 (6.57), 0.733 (7.64), 0.739 (6.32), 0.747 (3.40), 0.756 (1.41), 0.764 (1.90), 0.768 (3.40), 0.775 (5.79), 0.780 (4.77), 0.788 (3.89), 0.793 (2.87), 0.796 (6.03), 0.802 (3.99), 0.809 (1.31), 0.814 (1.75), 0.834 (0.49), 0.852 (0.78), 1.154 (1.46), 1.172 (2.82), 1.190 (1.46), 1.233 (2.92), 1.708 (0.97), 1.722 (1.80), 1.729 (1.90), 1.743 (2.87), 1.751 (1.17), 1.755 (1.80), 1.763 (1.70), 1.776 (0.83), 1.907 (0.58), 1.988 (4.13), 2.331 (2.14), 2.336 (0.97), 2.518 (16.00), 2.523 (10.75), 2.673 (2.14), 2.678 (0.97), 2.885 (0.88), 3.217 (0.44), 3.229 (0.63), 3.242 (0.58), 3.487 (7.54), 3.498 (6.86), 3.524 (1.07), 3.561 (8.32), 3.573 (9.14), 3.583 (5.16), 3.613 (1.17), 3.626 (1.26), 3.637 (1.02), 4.017 (0.83), 4.035 (0.83), 4.838 (5.40), 4.851 (5.45), 5.760 (13.67), 7.093 (0.97), 7.106 (3.31), 7.111 (2.67), 7.117 (3.70), 7.124 (5.06), 7.129 (4.04), 7.136 (2.82), 7.140 (3.55), 7.154 (1.07), 7.390 (2.77), 7.396 (1.80), 7.407 (2.63), 7.412 (2.24), 7.514 (2.48), 7.518 (2.53), 7.535 (2.38), 7.709 (15.95), 8.146 (0.53), 8.857 (2.04), 12.236 (3.36).

### Example 93

### 8-cyclopropyl-2-(morpholin-4-yl)-N-[(5-phenyl-4H-1,2,4-triazol-3-yl)methyl]pyrazolo[1,5-a][1,3,5]triazin-4-amine

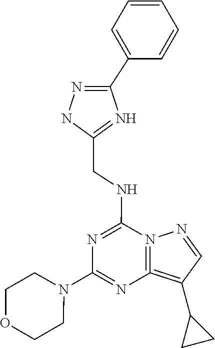

8-cyclopropyl-2-(methanesulfonyl)-N-[(5-phenyl-4H-1,2,4-triazol-3-yl)methyl]pyrazolo[1,5-a][1,3,5]triazin-4-amine (Intermediate 59, 108 mg) was provided in acetonitrile (2.0 mL), morpholine (230 μL, 2.6 mmol) was added and the mixture was stirred for 2 h at 70° C. The mixture was concentrated. The remaining material was purified by flash chromatography using silica gel (dichloromethane/ethanol gradient) to give 63.0 mg of the title compound.

LC-MS (Method 1): Rₜ=1.09 min; MS (ESIpos): m/z=418 [M+H]⁺

¹H-NMR (400 MHz, DMSO-d6) δ[ppm]: 0.700 (1.60), 0.702 (1.02), 0.712 (5.19), 0.717 (6.39), 0.724 (7.55), 0.730 (6.75), 0.738 (3.05), 0.747 (1.09), 0.756 (1.60), 0.760 (3.27), 0.767 (5.77), 0.773 (4.32), 0.781 (3.88), 0.785 (2.76), 0.788 (5.99), 0.794 (3.88), 0.800 (1.16), 0.806 (1.71), 0.851 (0.47), 1.232 (1.34), 1.697 (0.94), 1.710 (1.81), 1.718 (1.85), 1.731 (2.94), 1.739 (1.16), 1.743 (1.71), 1.752 (1.67), 1.764 (0.80), 2.327 (2.07), 2.331 (1.49), 2.336 (0.65), 2.518 (7.95), 2.523 (5.41), 2.669 (2.14), 2.673 (1.56), 2.678 (0.69), 3.538 (8.85), 3.549 (7.51), 3.603 (7.84), 3.614 (8.93), 3.624 (4.75), 4.735 (3.81), 4.746 (3.77), 7.399 (0.69), 7.417 (1.89), 7.434 (2.50), 7.447 (4.06), 7.466 (4.68), 7.483 (1.78), 7.691 (16.00), 7.944 (6.10), 7.947 (7.69), 7.959 (2.21), 7.964 (7.11), 7.968 (5.41), 8.823 (1.09).

### Example 94

### 8-cyclopropyl-2-(2,2-dimethylmorpholin-4-yl)-N-[(5-phenyl-4H-1,2,4-triazol-3-yl)methyl]pyrazolo[1,5-a][1,3,5]triazin-4-amine

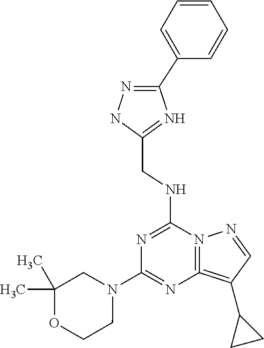

8-cyclopropyl-2-(methanesulfonyl)-N-[(5-phenyl-4H-1,2,4-triazol-3-yl)methyl]pyrazolo[1,5-a][1,3,5]triazin-4-amine (Intermediate 59, 108 mg) was dissolved in acetonitrile (3.0 mL), 2,2-dimethylmorpholine hydrogen chloride (1/1) (399 mg, 2.63 mmol; CAS-RN:[167946-94-3]) and N-ethyl-N-isopropylpropan-2-amine (460 μL, 2.6 mmol); CAS-RN:[7087-68-5]) were added and the mixture was stirred for 3 days at 40° C. The mixture was concentrated. The remaining material was purified by flash chromatography using silica gel (dichloromethane/ethanol gradient) to give 75.0 mg of the title compound.

LC-MS (Method 1): Rₜ=1.17 min; MS (ESIpos): m/z=446 [M+H]⁺

¹H-NMR (400 MHz, DMSO-d6) δ[ppm]: 0.693 (1.87), 0.698 (1.35), 0.705 (5.74), 0.710 (7.23), 0.717 (8.58), 0.723 (7.16), 0.731 (3.68), 0.745 (1.10), 0.750 (1.42), 0.758 (3.48), 0.766 (7.03), 0.771 (4.58), 0.779 (4.71), 0.783 (3.16), 0.787 (7.03), 0.792 (4.58), 0.798 (2.32), 0.804 (2.32), 0.815 (0.97), 0.822 (0.97), 0.835 (0.58), 0.840 (0.65), 0.852 (1.03), 0.886 (0.77), 0.905 (1.42), 0.923 (1.23), 0.978 (7.68), 1.027 (1.29), 1.035 (4.26), 1.053 (7.29), 1.070 (3.87), 1.085 (0.71), 1.104 (0.71), 1.113 (0.90), 1.129 (2.90), 1.168 (1.16), 1.189 (0.58), 1.203 (0.58), 1.233 (2.45), 1.352 (0.39), 1.692 (1.10), 1.705 (2.06), 1.713 (2.13), 1.725 (3.42), 1.734 (1.29), 1.739 (2.00), 1.747 (1.87), 1.760 (0.90), 1.907 (0.84), 1.987 (0.52), 2.005 (0.45), 2.066 (0.52), 2.147 (0.58), 2.318 (1.16), 2.456 (0.45), 2.461 (0.58), 2.518 (16.00), 2.523 (11.03), 2.537 (0.97), 2.541 (0.71), 2.660 (1.16), 3.405 (0.84), 3.422 (2.19), 3.435 (3.94), 3.440 (4.52), 3.452 (3.23), 3.547 (4.58), 3.559 (4.39), 3.611 (4.13), 4.343 (0.45), 4.355 (0.90), 4.368 (0.45), 4.711 (2.32), 5.759 (2.45), 7.455 (2.77), 7.511 (1.48), 7.532 (2.84), 7.552 (1.74), 7.675 (13.68), 7.697 (0.90), 7.873 (0.97), 7.877 (2.19), 7.880 (1.74), 7.886 (2.45), 7.889 (3.29), 7.895 (3.94), 7.946 (6.52), 7.964 (5.81), 8.847 (0.52).

### Example 95

### N-[(1H-benzimidazol-2-yl)methyl]-8-(2-fluorophenyl)-2-(morpholin-4-yl)pyrazolo[1,5-a][1,3,5]triazin-4-amine

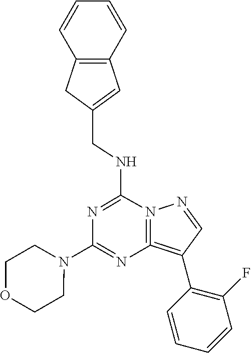

8-(2-fluorophenyl)-N-[(4-methoxyphenyl)methyl]-N-({1-[(4-methoxyphenyl)methyl]-1H-benzimidazol-2-yl}methyl)-2-(morpholin-4-yl)pyrazolo[1,5-a][1,3,5]triazin-4-amine (Intermediate 62, 10.0 mg) was dissolved in trifluoroacetic acid (1.0 mL) and was stirred for 4 h at 150° C. in the microwave. The mixture was concentrated to give a crude product. In a second preparation 8-(2-fluorophenyl)-N-[(4-methoxyphenyl)methyl]-N-({1-[(4-methoxyphenyl)methyl]-1H-benzimidazol-2-yl}methyl)-2-(morpholin-4-yl)pyrazolo[1,5-a][1,3,5]triazin-4-amine (Intermediate 62, 85.0 mg) was dissolved in trifluoroacetic acid (4.0 mL) and was stirred for 5 h at 150° C. in the microwave. The mixture was concentrated to give a crude product. The two crude products were combined and purified by flash chromatography using silica gel (dichloromethane/ethanol gradient) to give 48.0 mg of the title compound.

LC-MS (Method 1): Rₜ=1.03 min; MS (ESIpos): m/z=445 [M+H]⁺

¹H-NMR (400 MHz, DMSO-d6) δ[ppm]: −0.149 (0.56), −0.008 (7.08), 0.008 (4.98), 0.146 (0.56), 0.854 (0.64), 1.038 (5.63), 1.055 (12.70), 1.073 (6.27), 1.233 (3.06), 2.329 (4.66), 2.334 (3.38), 2.338 (1.53), 2.520 (16.00), 2.525 (10.93), 2.671 (4.74), 2.676 (3.38), 2.680 (1.45), 3.425 (1.13), 3.436 (1.29), 3.443 (1.21), 3.453 (1.29), 3.472 (0.88), 3.533 (5.63), 3.677 (7.72), 4.359 (0.72), 4.909 (11.74), 7.125 (4.74), 7.132 (5.07), 7.141 (5.23), 7.147 (5.07), 7.168 (0.96), 7.172 (0.88), 7.185 (1.45), 7.189 (2.01), 7.193 (2.25), 7.199 (2.25), 7.205 (3.94), 7.210 (3.06), 7.218 (2.97), 7.224 (3.94), 7.229 (4.82), 7.233 (5.07), 7.237 (3.70), 7.246 (2.57), 7.251 (4.82), 7.255 (8.12), 7.269 (2.73), 7.273 (2.89), 7.279 (1.69), 7.431 (1.13), 7.527 (1.13), 8.301 (11.26), 8.311 (10.69), 8.409 (2.09), 8.413 (2.41), 8.428 (3.54), 8.433 (4.02), 8.447 (1.85), 8.452 (2.25), 8.556 (0.56), 9.169 (1.13), 12.298 (1.29).

### Example 96

### 8-bromo-N-{[5-(2-chlorophenyl)-1H-imidazol-2-yl]methyl}-2-(morpholin-4-yl)pyrazolo[1,5-a][1,3,5]triazin-4-amine

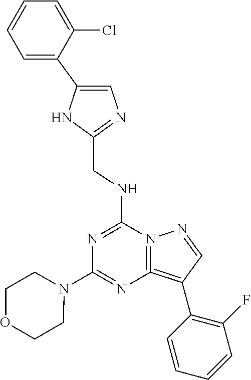

{[8-bromo-2-(morpholin-4-yl)pyrazolo[1,5-a][1,3,5]triazin-4-yl]amino}ethanimidamide hydrogen chloride (1/1) (Intermediate 48, 150 mg, 383 μmol) and 2-bromo-1-(2-chlorophenyl)ethan-1-one (116 mg, 498 μmol) were dissolved in N,N-dimethylformamide (2.5 mL), cesium carbonate (499 mg, 1.53 mmol) was added and the mixture was stirred over night at 60° C. The mixture was filtered and purified by preparative HPLC twice (1. HT basic, 2. see below) to give 3.7 mg (2% yield) of the title compound.

### Preparative HPLC Method

Instrument: Waters Autopurificationsystem; Column: Waters XBrigde C18 5μ 50×50 mm; eluent A: water+0.1 Vol-% formic acid (99%), eluent B: acetonitrile; gradient: 0.00-0.50 min 27% B (50-100 mL/min), 0.51-8.50 min 27-47% B (100 mL/min), DAD scan: 210-400 nm.

### Analytical HPLC Method

Instrument: Waters Acquity UPLCMS SingleQuad; Column: Acquity UPLC BEH C18 1.7 μm, 50×2.1 mm; eluent A: water+0.1 Vol-% formic acid (99%), eluent B: acetonitrile; gradient: 0-2.6 min 1-99% B, 2.6-3.0 min 99% B; flow 0.8 mL/min; temperature: 60° C.; DAD scan: 210-400 nm.

Analytical HPLC: Rₜ=0.97 min.

LC-MS (Method 2): Rₜ=1.19 min; MS (ESIpos): m/z=489 [M+H]⁺

¹H-NMR (400 MHz, DMSO-d6) δ[ppm]: −0.149 (0.54), −0.008 (5.87), 0.008 (4.17), 0.146 (0.46), 1.234 (1.16), 1.892 (0.39), 2.320 (1.47), 2.324 (2.78), 2.329 (3.63), 2.334 (2.63), 2.520 (11.59), 2.525 (7.73), 2.542 (2.40), 2.662 (1.39), 2.666 (2.78), 2.671 (3.63), 2.676 (2.55), 2.680 (1.16), 3.567 (7.65), 3.674 (8.81), 3.687 (9.82), 3.698 (5.49), 4.736 (5.80), 7.185 (1.55), 7.189 (1.55), 7.205 (2.78), 7.208 (2.71), 7.223 (2.32), 7.227 (2.16), 7.289 (0.54), 7.325 (2.16), 7.328 (2.24), 7.346 (3.17), 7.363 (1.70), 7.367 (1.70), 7.435 (4.02), 7.437 (3.79), 7.455 (3.40), 7.458 (3.17), 7.660 (6.42), 8.015 (16.00), 8.060 (3.40), 8.064 (3.40), 8.080 (3.17), 8.084 (3.01), 9.018 (1.31), 12.120 (1.78).

### Example 97

### 8-bromo-N-[(4-fluoro-1H-benzimidazol-2-yl)methyl]-2-(morpholin-4-yl)pyrazolo[1,5-a][1,3,5]triazin-4-amine

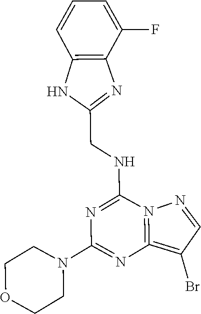

8-bromo-N-[(4-fluoro-1H-benzimidazol-2-yl)methyl]-2-(methanesulfonyl)pyrazolo[1,5-a][1,3,5]triazin-4-amine (Intermediate 64, 200 mg) and morpholine (83 μL, 950 μmol) were provided in acetonitrile (2.0 mL), N-ethyl-N-isopropylpropan-2-amine (170 μL, 950 μmol; CAS-RN:[7087-68-5]) was added and the mixture was stirred over night at 70° C. The mixture was poured into water and extracted with ethyl acetate. The combined organic layers were washed with water, dried over sodium sulfate, filtered and concentrated. The remaining material was purified by preparative HPLC (HT basic) to give 35.4 mg of the title compound.

LC-MS (Method 2): Rₜ=1.04 min; MS (ESIpos): m/z=447 [M+H]⁺

¹H-NMR (400 MHz, DMSO-d6) δ[ppm]: 1.232 (0.43), 2.331 (2.17), 2.336 (1.03), 2.518 (11.12), 2.522 (7.59), 2.539 (0.49), 2.673 (2.22), 2.678 (0.98), 3.487 (5.59), 3.616 (8.35), 4.875 (6.62), 4.887 (6.45), 6.917 (1.41), 6.936 (1.95), 6.943 (1.74), 6.964 (1.79), 7.099 (1.30), 7.111 (1.68), 7.119 (2.66), 7.131 (2.77), 7.139 (1.63), 7.151 (1.46), 7.236 (3.69), 7.256 (2.66), 7.400 (0.38), 8.041 (16.00), 9.185 (1.74), 12.576 (2.06).

### Example 98

### 8-bromo-N-{[5-(3-fluoro-4-methylphenyl)-4H-1,2,4-triazol-3-yl]methyl}-2-(morpholin-4-yl)pyrazolo[1,5-a][1,3,5]triazin-4-amine

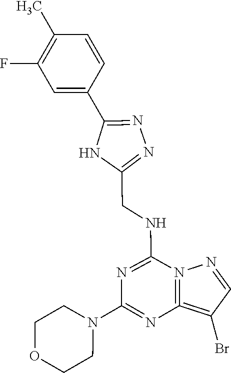

2-{[8-bromo-2-(morpholin-4-yl)pyrazolo[1,5-a][1,3,5]triazin-4-yl]amino}acetohydrazide (Intermediate 39, 100 mg, 269 μmol) and 3-fluoro-4-methylbenzene-1-carboximidamide hydrogen chloride (1/1) (61.0 mg, 323 μmol) were dissolved in DMF (2.5 mL), sodium ethanolate (36.7 mg, 539 μmol) was added and the mixture was stirred in a sealed tube for 45 min at 180° C. in the microwave. The mixture was filtered and purified by preparative HPLC (HT basic) to give 34.3 mg (23% yield) of the title compound.

LC-MS (Method 2): Rₜ=0.90 min; MS (ESIpos): m/z=488 [M+H]⁺

¹H-NMR (400 MHz, DMSO-d6) δ[ppm]: −0.149 (0.61), −0.008 (5.74), 0.008 (4.95), 0.146 (0.55), 2.267 (11.18), 2.270 (10.81), 2.314 (1.47), 2.338 (1.10), 2.464 (2.87), 2.469 (3.54), 2.520 (11.30), 2.525 (7.51), 2.680 (1.16), 3.547 (4.76), 3.650 (5.31), 3.662 (6.17), 4.766 (4.52), 7.363 (1.04), 7.383 (2.14), 7.403 (1.16), 7.606 (0.43), 7.617 (2.08), 7.620 (2.20), 7.647 (2.14), 7.688 (3.18), 7.692 (2.75), 7.708 (2.56), 7.712 (2.38), 8.024 (16.00), 9.121 (0.55).

### Example 99

### 8-bromo-N-{[5-(3,4-difluorophenyl)-4H-1,2,4-triazol-3-yl]methyl}-2-(morpholin-4-yl)pyrazolo[1,5-a][1,3,5]triazin-4-amine

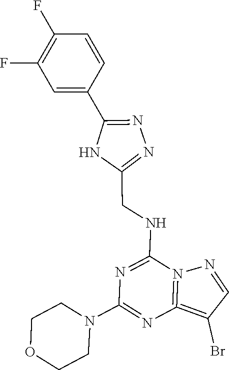

2-{[8-bromo-2-(morpholin-4-yl)pyrazolo[1,5-a][1,3,5]triazin-4-yl]amino}acetohydrazide (Intermediate 39, 100 mg, 269 μmol) and 3,4-difluorobenzene-1-carboximidamide hydrogen chloride (1/1) (62.3 mg, 323 μmol) were dissolved in DMF (2.5 mL), sodium ethanolate (36.7 mg, 539 μmol) was added and the mixture was stirred in a sealed tube for 45 min at 180° C. in the microwave. The mixture was filtered and purified by preparative HPLC (HT basic) to give 15.6 mg (11% yield) of the title compound.

LC-MS (Method 2): Rₜ=0.88 min; MS (ESIpos): m/z=491 [M+H]⁺

¹H-NMR (400 MHz, DMSO-d6) δ[ppm]: −0.008 (3.21), 0.008 (3.01), 2.325 (2.11), 2.329 (2.86), 2.334 (2.11), 2.525 (10.58), 2.667 (2.16), 2.672 (2.91), 2.676 (2.16), 2.848 (0.90), 2.983 (0.95), 2.987 (0.95), 3.548 (7.77), 3.643 (8.13), 3.654 (9.53), 4.782 (9.23), 7.507 (1.00), 7.529 (2.61), 7.552 (2.76), 7.576 (1.25), 7.800 (2.81), 7.822 (2.41), 7.869 (1.50), 7.893 (2.61), 7.918 (1.55), 7.922 (1.40), 8.002 (0.45), 8.028 (16.00), 9.142 (0.70).

### Example 100

### 8-bromo-2-(morpholin-4-yl)-N-{[5-(3,4,5-trifluorophenyl)-4H-1,2,4-triazol-3-yl]methyl}pyrazolo[1,5-a][1,3,5]triazin-4-amine

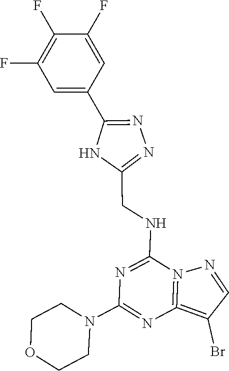

2-{[8-bromo-2-(morpholin-4-yl)pyrazolo[1,5-a][1,3,5]triazin-4-yl]amino}acetohydrazide (Intermediate 39, 100 mg, 269 μmol) and 3,4,5-trifluorobenzene-1-carboximidamide hydrogen chloride (1/1) (68.1 mg, 323 μmol) were dissolved in DMF (2.5 mL), sodium ethanolate (36.7 mg, 539 μmol) was added and the mixture was stirred in a sealed tube for 45 min at 180° C. in the microwave. The mixture was filtered and purified by preparative HPLC (HT basic) to give 9.4 mg (7% yield) of the title compound.

LC-MS (Method 2): Rₜ=0.84 min; MS (ESIpos): m/z=510 [M+H]⁺

¹H-NMR (400 MHz, DMSO-d6) δ[ppm]: −0.008 (4.16), 0.008 (3.10), 0.146 (0.42), 1.900 (0.51), 2.329 (2.55), 2.334 (1.82), 2.339 (0.81), 2.521 (8.06), 2.525 (5.56), 2.542 (0.47), 2.667 (1.87), 2.671 (2.55), 2.676 (1.78), 2.945 (0.42), 3.550 (5.52), 3.636 (5.81), 3.647 (6.49), 4.793 (8.49), 7.763 (3.06), 7.779 (3.61), 7.784 (3.69), 7.802 (3.31), 7.812 (0.47), 8.030 (16.00).

### Example 101

### 8-bromo-N-[(5-fluoro-1H-benzimidazol-2-yl)methyl]-2-(morpholin-4-yl)pyrazolo[1,5-a][1,3,5]triazin-4-amine

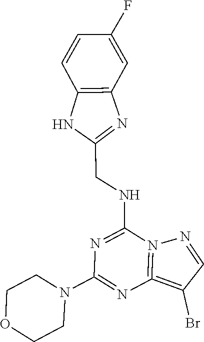

8-bromo-N-[(5-fluoro-1H-benzimidazol-2-yl)methyl]-2-(methanesulfonyl)pyrazolo[1,5-a][1,3,5]triazin-4-amine (Intermediate 66, 200 mg, 454 μmol) and morpholine (120 μL, 1.4 mmol) were provided in acetonitrile (10 mL), N-ethyl-N-isopropylpropan-2-amine (240 μL, 1.4 mmol) was added and the mixture was stirred over night at 70° C. The mixture was poured into water and extracted with ethyl acetate. The combined organic layers were washed with water, dried over sodium sulfate, filtered and concentrated. The remaining material was purified by preparative HPLC (HT basic) to give 35.5 mg (17% yield) of the title compound.

LC-MS (Method 2): Rₜ=1.04 min; MS (ESIpos): m/z=447 [M+H]⁺

¹H-NMR (400 MHz, DMSO-d6) δ[ppm]: −0.149 (0.31), −0.008 (3.06), 0.008 (2.37), 0.146 (0.28), 1.234 (0.21), 1.873 (0.21), 2.076 (0.49), 2.324 (1.50), 2.329 (2.02), 2.334 (1.46), 2.338 (0.66), 2.520 (6.92), 2.525 (4.49), 2.542 (1.53), 2.666 (1.46), 2.671 (1.98), 2.676 (1.43), 2.680 (0.63), 3.497 (3.48), 3.618 (5.18), 4.858 (8.42), 6.966 (0.83), 6.972 (0.87), 6.994 (1.50), 7.013 (0.87), 7.018 (0.87), 7.278 (0.49), 7.317 (0.42), 7.322 (0.42), 7.463 (0.49), 7.995 (0.21), 8.013 (0.21), 8.039 (16.00), 8.054 (0.45), 9.148 (0.31), 12.392 (0.28).

### Example 102

### N-[(1H-benzimidazol-2-yl)methyl]-8-bromo-2-[(1R,4R)-2-oxa-5-azabicyclo[2.2.1]heptan-5-yl]pyrazolo[1,5-a][1,3,5]triazin-4-amine

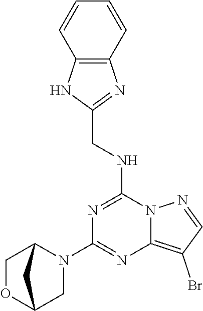 [SMILES: Brc1cnn2c(NCc3nc4ccccc4[nH]3)nc(N3C[C@H]4C[C@@H]3CO4)nc12]

N-[(1H-benzimidazol-2-yl)methyl]-8-bromo-2-(methanesulfonyl)pyrazolo[1,5-a][1,3,5]triazin-4-amine (Intermediate 9, 100 mg, 213 μmol) was suspended in acetonitrile (2.0 mL), (1R,4R)-2-oxa-5-azabicyclo[2.2.1]heptane hydrogen chloride (1/1) (86.7 mg, 639 μmol) and N-ethyl-N-isopropylpropan-2-amine (110 μL, 640 μmol) were added and the mixture was stirred for 18 h at 70° C. in a sealed tube. The mixture was diluted with water and extracted with ethyl acetate. The combined organic layers were filtered, dried and concentrated. The remaining material was purified by preparative HPLC (HT basic) to give 50.9 mg (52% yield) of the title compound.

Specific Optical Rotation (Method O1): +48.8° (c=10 mg/mL, DMSO) LC-MS (Method 2): Rₜ=0.95 min; MS (ESIpos): m/z=441 [M+H]⁺

¹H-NMR (400 MHz, DMSO-d6) δ[ppm]: −0.009 (1.30), 0.008 (0.81), 1.686 (0.43), 1.709 (0.68), 1.775 (0.84), 1.803 (0.93), 2.329 (0.81), 2.334 (0.59), 2.520 (2.39), 2.525 (1.80), 2.542 (5.27), 2.676 (0.56), 3.222 (0.65), 3.240 (0.99), 3.286 (1.46), 3.379 (1.24), 3.405 (0.50), 4.559 (2.29), 4.722 (1.18), 4.864 (2.23), 7.096 (0.47), 7.115 (1.46), 7.127 (2.33), 7.143 (1.52), 7.158 (0.47), 7.387 (0.90), 7.404 (1.05), 7.524 (1.30), 7.542 (1.15), 8.020 (16.00), 9.127 (0.65), 12.237 (1.83).

### Example 103

### N-[(1H-benzimidazol-2-yl)methyl]-8-bromo-2-[(1S,4S)-2-oxa-5-azabicyclo[2.2.1]heptan-5-yl]pyrazolo[1,5-a][1,3,5]triazin-4-amine

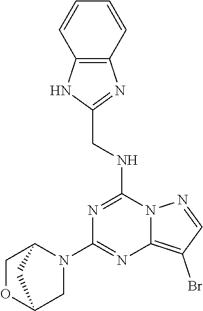 [SMILES: Brc1cnn2c(NCc3nc4ccccc4[nH]3)nc(N3C[C@@H]4C[C@H]3CO4)nc12]

N-[(1H-benzimidazol-2-yl)methyl]-8-bromo-2-(methanesulfonyl)pyrazolo[1,5-a][1,3,5]triazin-4-amine (Intermediate 9, 100 mg, 213 μmol) was suspended in acetonitrile (2.0 mL), (1S,4S)-2-oxa-5-azabicyclo[2.2.1]heptane hydrogen chloride (1/1) (86.7 mg, 639 μmol) and N-ethyl-N-isopropylpropan-2-amine (110 μL, 640 μmol) were added and the mixture was stirred for 18 h at 70° C. in a sealed tube. The mixture was diluted with water and extracted with ethyl acetate. The combined organic layers were filtered, dried and concentrated. The remaining material was purified by preparative HPLC (HT basic) to give 55.1 mg (56% yield) of the title compound.

Specific Optical Rotation (Method O1): −43.7° (c=10 mg/mL, DMSO)

LC-MS (Method 2): Rₜ=0.95 min; MS (ESIpos): m/z=441 [M+H]⁺

¹H-NMR (400 MHz, DMSO-d6) δ[ppm]: −0.008 (1.00), 0.008 (0.89), 1.685 (0.53), 1.708 (0.83), 1.776 (1.01), 1.802 (1.14), 2.334 (0.53), 2.520 (2.23), 2.525 (1.63), 2.542 (4.72), 2.676 (0.51), 3.223 (0.75), 3.241 (1.14), 3.288 (1.51), 3.304 (1.51), 3.379 (1.40), 3.405 (0.55), 3.691 (0.46), 3.762 (0.48), 4.559 (2.75), 4.722 (1.41), 4.865 (2.68), 7.096 (0.56), 7.115 (1.78), 7.127 (2.83), 7.141 (1.87), 7.159 (0.58), 7.387 (1.10), 7.405 (1.29), 7.524 (1.58), 7.542 (1.43), 8.021 (16.00), 8.035 (0.41), 9.128 (0.85), 12.237 (2.25).

### Example 104

### N-[(1H-benzimidazol-2-yl)methyl]-2-(morpholin-4-yl)-8-phenylpyrazolo[1,5-a][1,3,5]triazin-4-amine

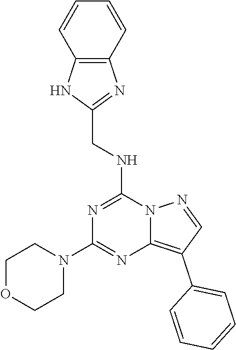 [SMILES: c1ccc(-c2cnn3c(NCc4nc5ccccc5[nH]4)nc(N4CCOCC4)nc23)cc1]

N-[(4-methoxyphenyl)methyl]-N-({1-[(4-methoxyphenyl)methyl]-1H-benzimidazol-2-yl}methyl)-2-(morpholin-4-yl)-8-phenylpyrazolo[1,5-a][1,3,5]triazin-4-amine (Intermediate 67, 160 mg, 240 μmol) was dissolved in trifluoroacetic acid (4.0 mL) and was stirred for 5 h at 150° C. in the microwave. The mixture was concentrated and purified by flash chromatography using silica gel (aminophase, dichloromethane/ethanol gradient) to give 57.0 mg (53% yield) of the title compound.

LC-MS (Method 1): Rₜ=0.99 min; MS (ESIpos): m/z=427 [M+H]⁺

¹H-NMR (400 MHz, DMSO-d6) δ[ppm]: −0.008 (1.82), 0.008 (1.54), 1.234 (0.52), 1.990 (0.63), 2.329 (1.04), 2.334 (0.77), 2.520 (3.63), 2.525 (2.32), 2.671 (1.07), 2.676 (0.75), 3.542 (3.58), 3.684 (4.45), 3.709 (2.22), 4.898 (3.83), 4.907 (3.65), 5.762 (2.29), 7.108 (1.29), 7.110 (2.07), 7.113 (1.54), 7.129 (6.06), 7.144 (4.11), 7.147 (4.20), 7.150 (3.04), 7.346 (4.42), 7.351 (1.59), 7.366 (6.15), 7.381 (1.43), 7.386 (3.95), 7.411 (0.97), 7.424 (0.95), 7.536 (1.00), 8.019 (5.15), 8.022 (6.10), 8.035 (1.68), 8.040 (5.47), 8.043 (4.58), 8.431 (0.43), 8.494 (16.00), 9.077 (1.29), 12.289 (1.73).

### Example 105

### 8-bromo-N-{[5-(3,5-difluorophenyl)-4H-1,2,4-triazol-3-yl]methyl}-2-(morpholin-4-yl)pyrazolo[1,5-a][1,3,5]triazin-4-amine

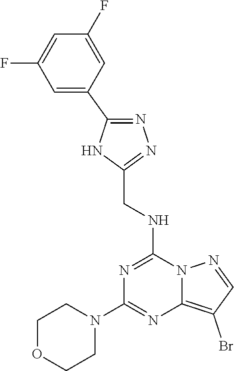 [SMILES: Fc1cc(F)cc(-c2nnc(CNc3nc(N4CCOCC4)nc4c(Br)cnn34)[nH]2)c1]

2-{[8-bromo-2-(morpholin-4-yl)pyrazolo[1,5-a][1,3,5]triazin-4-yl]amino}acetohydrazide (Intermediate 39, 100 mg, 269 μmol) and 3,5-difluorobenzene-1-carboximidamide hydrogen chloride (1/1) (62.3 mg, 323 μmol) were dissolved in DMF (2.5 mL), sodium ethanolate (36.7 mg, 539 μmol) was added and the mixture was stirred in a sealed tube for 45 min at 180° C. in the microwave. The mixture was filtered and purified by preparative HPLC (HT basic) to give 69.4 mg (47% yield) of the title compound.

LC-MS (Method 2): Rₜ=0.85 min; MS (ESIpos): m/z=492 [M+H]⁺

¹H-NMR (400 MHz, DMSO-d6) δ[ppm]: −0.008 (1.31), 0.008 (1.20), 1.906 (0.41), 2.521 (5.05), 2.526 (3.39), 2.542 (0.48), 2.681 (0.44), 3.548 (5.53), 3.640 (5.97), 3.651 (6.87), 3.686 (0.81), 3.699 (0.81), 4.069 (0.41), 4.798 (6.94), 7.301 (1.06), 7.306 (0.74), 7.318 (1.15), 7.323 (2.10), 7.330 (1.36), 7.341 (0.62), 7.347 (1.06), 7.353 (0.65), 7.560 (0.53), 7.572 (3.16), 7.577 (4.15), 7.581 (2.70), 7.593 (4.01), 7.598 (3.23), 7.611 (0.58), 8.002 (1.31), 8.031 (16.00), 8.040 (0.41), 9.163 (0.58).

### Example 106

### N-[(1H-benzimidazol-2-yl)methyl]-8-(4-fluorophenyl)-2-(morpholin-4-yl)pyrazolo[1,5-a][1,3,5]triazin-4-amine

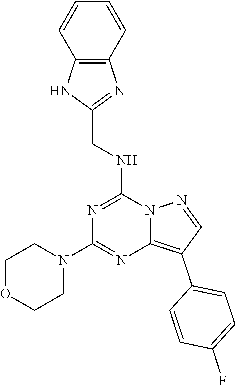 [SMILES: Fc1ccc(-c2cnn3c(NCc4nc5ccccc5[nH]4)nc(N4CCOCC4)nc23)cc1]

8-(4-fluorophenyl)-N-[(4-methoxyphenyl)methyl]-N-({1-[(4-methoxyphenyl)methyl]-1H-benzimidazol-2-yl}methyl)-2-(morpholin-4-yl)pyrazolo[1,5-a][1,3,5]triazin-4-amine (Intermediate 68, 140 mg) was dissolved in trifluoroacetic acid (4.0 mL) and was stirred for 5 h at 150° C. in the microwave. The mixture was concentrated and purified by flash chromatography twice using silica gel (dichloromethane/ethanol gradient) to give 48.0 mg of the title compound.

LC-MS (Method 1): Rₜ=1.01 min; MS (ESIpos): m/z=445 [M+H]⁺

¹H-NMR (400 MHz, DMSO-d6) δ[ppm]: −0.008 (0.86), 0.008 (0.78), 1.038 (8.42), 1.055 (16.00), 1.073 (9.01), 1.234 (0.53), 2.334 (0.46), 2.338 (0.20), 2.520 (2.45), 2.525 (1.53), 2.676 (0.46), 2.680 (0.21), 3.408 (0.45), 3.426 (1.26), 3.436 (1.36), 3.443 (1.32), 3.453 (1.28), 3.471 (0.52), 3.539 (1.94), 3.678 (2.49), 4.347 (0.58), 4.359 (0.99), 4.371 (0.56), 4.899 (3.68), 5.761 (0.35), 7.124 (1.67), 7.132 (1.70), 7.139 (1.74), 7.146 (1.82), 7.156 (0.41), 7.174 (0.26), 7.182 (2.11), 7.187 (0.73), 7.199 (0.88), 7.205 (4.35), 7.210 (0.86), 7.222 (0.72), 7.227 (2.12), 7.234 (0.23), 7.503 (0.32), 8.034 (0.26), 8.041 (2.10), 8.047 (0.90), 8.055 (2.30), 8.064 (2.34), 8.072 (0.83), 8.078 (2.05), 8.085 (0.21), 8.486 (8.20), 9.086 (0.32), 12.288 (0.34).

### Example 107

### N-[(1H-benzimidazol-2-yl)methyl]-8-(3-chlorophenyl)-2-(morpholin-4-yl)pyrazolo[1,5-a][1,3,5]triazin-4-amine

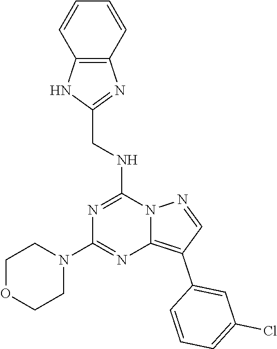 [SMILES: Clc1cccc(-c2cnn3c(NCc4nc5ccccc5[nH]4)nc(N4CCOCC4)nc23)c1]

8-(3-chlorophenyl)-N-[(4-methoxyphenyl)methyl]-N-({1-[(4-methoxyphenyl)methyl]-1H-benzimidazol-2-yl}methyl)-2-(morpholin-4-yl)pyrazolo[1,5-a][1,3,5]triazin-4-amine (Intermediate 69, 183 mg, 261 μmol) was dissolved in trifluoroacetic acid (4.0 mL) and was stirred for 5 h at 150° C. in the microwave. The mixture was concentrated and purified by flash chromatography twice using silica gel (dichloromethane/ethanol gradient) to give 45.0 mg (34% yield) of the title compound.

LC-MS (Method 1): Rₜ=1.13 min; MS (ESIpos): m/z=461 [M+H]⁺

¹H-NMR (400 MHz, DMSO-d6) δ[ppm]: −0.008 (2.98), 0.008 (2.66), 0.807 (0.67), 0.816 (2.06), 0.824 (0.83), 0.833 (4.68), 0.838 (2.74), 0.843 (1.27), 0.855 (3.30), 0.861 (2.74), 0.871 (1.03), 0.877 (0.83), 0.938 (1.95), 0.955 (1.99), 1.157 (2.30), 1.175 (4.61), 1.192 (2.34), 1.240 (1.83), 1.277 (0.64), 1.295 (0.40), 1.397 (2.10), 1.990 (8.10), 2.329 (2.34), 2.334 (1.71), 2.520 (10.00), 2.525 (6.07), 2.671 (2.38), 2.676 (1.71), 3.545 (4.25), 3.572 (2.38), 3.687 (6.07), 3.712 (3.14), 3.795 (0.60), 3.972 (0.48), 4.002 (0.71), 4.019 (1.99), 4.037 (1.95), 4.055 (0.67), 4.894 (6.67), 4.909 (6.59), 6.872 (0.40), 7.106 (1.23), 7.120 (3.65), 7.124 (3.33), 7.130 (4.09), 7.137 (6.51), 7.143 (4.45), 7.149 (6.00), 7.154 (5.76), 7.170 (3.65), 7.172 (3.77), 7.175 (3.41), 7.177 (2.90), 7.370 (3.81), 7.389 (6.99), 7.409 (4.92), 7.422 (3.26), 7.426 (2.62), 7.534 (2.94), 7.538 (3.02), 7.549 (1.83), 7.555 (2.78), 8.012 (3.45), 8.033 (3.26), 8.099 (4.33), 8.104 (6.55), 8.108 (4.05), 8.536 (0.56), 8.579 (16.00), 9.119 (1.67), 9.133 (3.45), 9.148 (1.55), 12.286 (4.21).

### Example 108

### N-[(1H-benzimidazol-2-yl)methyl]-2-(morpholin-4-yl)-8-(pyridin-3-yl)pyrazolo[1,5-a][1,3,5]triazin-4-amine

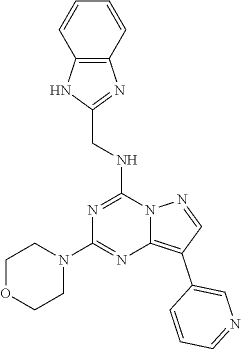 [SMILES: c1cncc(-c2cnn3c(NCc4nc5ccccc5[nH]4)nc(N4CCOCC4)nc23)c1]

N-[(4-methoxyphenyl)methyl]-N-({1-[(4-methoxyphenyl)methyl]-1H-benzimidazol-2-yl}methyl)-2-(morpholin-4-yl)-8-(pyridin-3-yl)pyrazolo[1,5-a][1,3,5]triazin-4-amine (Intermediate 70, 183 mg, 274 μmol) was dissolved in trifluoroacetic acid (4.2 mL) and was stirred for 5 h at 150° C. in the microwave. The mixture was concentrated and purified by flash chromatography twice using silica gel (dichloromethane/ethanol gradient) to give 87.0 mg (72% yield) of the title compound.

LC-MS (Method 2): Rₜ=0.90 min; MS (ESIpos): m/z=428 [M+H]⁺

¹H-NMR (400 MHz, DMSO-d6) δ[ppm]: −0.008 (1.10), 0.008 (0.91), 1.234 (0.43), 2.330 (0.71), 2.334 (0.51), 2.521 (2.50), 2.526 (1.58), 2.672 (0.73), 2.676 (0.53), 3.543 (2.64), 3.691 (3.57), 4.907 (6.85), 7.127 (2.80), 7.134 (2.92), 7.142 (2.99), 7.149 (3.09), 7.159 (0.70), 7.368 (2.14), 7.370 (2.27), 7.379 (2.22), 7.382 (2.24), 7.387 (2.33), 7.389 (2.28), 7.399 (2.49), 7.401 (2.50), 7.440 (0.55), 7.522 (0.55), 8.324 (3.71), 8.328 (4.43), 8.336 (3.56), 8.340 (4.01), 8.360 (2.13), 8.364 (2.47), 8.370 (1.89), 8.380 (2.03), 8.386 (2.48), 8.390 (1.59), 8.596 (16.00), 9.152 (0.58), 9.233 (4.26), 9.235 (4.33), 9.238 (4.49), 9.240 (4.01), 12.290 (0.59).

### Example 109

### 8-bromo-2-[(2R,6S)-2,6-dimethylmorpholin-4-yl]-N-[(4-fluoro-1H-benzimidazol-2-yl)methyl]pyrazolo[1,5-a][1,3,5]triazin-4-amine

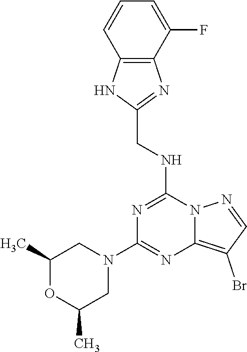 [SMILES: C[C@@H]1CN(c2nc(NCc3nc4c(F)cccc4[nH]3)n3ncc(Br)c3n2)C[C@H](C)O1]

8-bromo-N-[(4-fluoro-1H-benzimidazol-2-yl)methyl]-2-(methanesulfonyl)pyrazolo[1,5-a][1,3,5]triazin-4-amine (Intermediate 64, 200 mg) and (2R,6S)-2,6-dimethylmorpholine (170 μL, 1.4 mmol) were provided in acetonitrile (10 mL), N-ethyl-N-isopropylpropan-2-amine (240 μL, 1.4 mmol) was added and the mixture was stirred over night at 70° C. The mixture was concentrated and purified by preparative HPLC (HT basic) to give 42.4 mg of the title compound.

LC-MS (Method 2): Rₜ=1.18 min; MS (ESIpos): m/z=475 [M+H]⁺

¹H-NMR (400 MHz, DMSO-d6) δ[ppm]: −0.008 (3.21), 0.008 (2.42), 1.016 (3.02), 1.111 (1.72), 1.126 (1.35), 2.325 (2.37), 2.330 (2.88), 2.334 (2.37), 2.521 (6.88), 2.525 (4.33), 2.542 (9.16), 2.667 (1.35), 2.672 (1.86), 2.676 (1.35), 4.302 (0.79), 4.859 (6.56), 4.871 (6.23), 6.913 (2.19), 6.933 (2.79), 6.940 (2.37), 6.961 (2.56), 7.005 (0.56), 7.031 (0.47), 7.097 (1.77), 7.109 (2.05), 7.118 (3.40), 7.129 (3.21), 7.137 (1.95), 7.149 (1.67), 7.225 (5.77), 7.244 (4.05), 7.373 (0.84), 7.392 (0.70), 8.040 (16.00), 8.054 (0.42), 9.151 (0.65), 9.179 (1.63), 9.191 (2.74), 12.597 (3.40), 12.911 (0.70).

### Example 110

### 8-bromo-2-[(2R,6S)-2,6-dimethylmorpholin-4-yl]-N-[(5-fluoro-1H-benzimidazol-2-yl)methyl]pyrazolo[1,5-a][1,3,5]triazin-4-amine

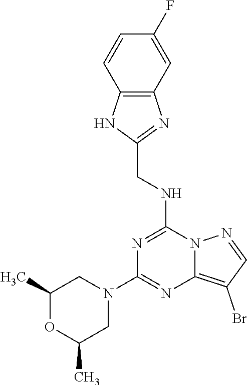 [SMILES: C[C@@H]1CN(c2nc(NCc3nc4cc(F)ccc4[nH]3)n3ncc(Br)c3n2)C[C@H](C)O1]

8-bromo-N-[(5-fluoro-1H-benzimidazol-2-yl)methyl]-2-(methanesulfonyl)pyrazolo[1,5-a][1,3,5]triazin-4-amine (Intermediate 66, 200 mg, 454 μmol) and (2R,6S)-2,6-dimethylmorpholine (170 μL, 1.4 mmol) were provided in acetonitrile (13 mL), N-ethyl-N-isopropylpropan-2-amine (240 μL, 1.4 mmol) was added and the mixture was stirred over night at 70° C. The mixture was concentrated and purified by preparative HPLC (HT basic) to give 32.6 mg (14% yield) of the title compound.

LC-MS (Method 2): Rₜ=1.18 min; MS (ESIpos): m/z=475 [M+H]⁺

¹H-NMR (400 MHz, DMSO-d6) δ[ppm]: −0.149 (0.25), −0.008 (2.60), 0.008 (1.98), 0.146 (0.20), 1.024 (1.66), 1.090 (1.36), 1.105 (1.09), 1.137 (0.67), 1.263 (0.25), 1.356 (0.27), 1.363 (0.25), 2.320 (0.84), 2.324 (1.43), 2.329 (1.90), 2.334 (1.48), 2.338 (0.94), 2.520 (4.28), 2.525 (3.07), 2.542 (2.94), 2.662 (0.52), 2.667 (1.06), 2.671 (1.46), 2.676 (1.04), 2.680 (0.45), 4.332 (0.47), 4.848 (4.40), 6.963 (0.79), 6.969 (0.87), 6.992 (1.46), 7.010 (0.89), 7.016 (0.89), 7.268 (0.45), 7.441 (0.42), 7.988 (0.20), 8.008 (0.20), 8.034 (16.00), 8.048 (0.62), 9.148 (0.40), 12.403 (0.35).

### Example 111

### N-[(1H-benzimidazol-2-yl)methyl]-2-(morpholin-4-yl)-8-(pyridin-4-yl)pyrazolo[1,5-a][1,3,5]triazin-4-amine hydrogen chloride (1/1)

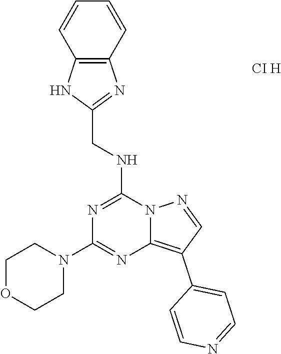 [SMILES: c1ccc2[nH]c(CNc3nc(N4CCOCC4)nc4c(-c5ccncc5)cnn34)nc2c1 ; "CI H"]

N-[(4-methoxyphenyl)methyl]-N-({1-[(4-methoxyphenyl)methyl]-1H-benzimidazol-2-yl}methyl)-2-(morpholin-4-yl)-8-(pyridin-4-yl)pyrazolo[1,5-a][1,3,5]triazin-4-amine (Intermediate 71, 185 mg, 277 μmol) was dissolved in trifluoroacetic acid (4.2 mL) and was stirred for 5 h at 150° C. in the microwave. The mixture was concentrated and purified by flash chromatography using silica gel (dichloromethane/ethanol gradient). The product was washed with water and dichloromethane, a hydrogen chloride solution in dioxane (0.5 mL, 4 M) was added and the solvent was removed by evaporation to give 20.0 mg (15% yield) of the title compound.

LC-MS (Method 1): Rₜ=0.59 min; MS (ESIpos): m/z=428 [M+H]⁺

¹H-NMR (400 MHz, DMSO-d6) δ[ppm]: −0.149 (0.52), −0.008 (4.64), 0.008 (4.71), 0.146 (0.52), 0.854 (0.60), 1.234 (3.59), 1.909 (0.82), 2.324 (2.99), 2.329 (4.11), 2.334 (3.07), 2.338 (1.35), 2.520 (15.55), 2.525 (9.64), 2.662 (1.42), 2.666 (2.99), 2.671 (4.19), 2.676 (3.07), 2.680 (1.35), 3.483 (6.58), 3.491 (6.65), 3.502 (6.88), 3.567 (8.00), 3.679 (1.64), 3.702 (1.27), 3.716 (1.05), 3.845 (1.50), 5.138 (4.49), 5.151 (4.49), 7.450 (2.77), 7.456 (3.07), 7.463 (3.21), 7.471 (3.14), 7.710 (4.19), 7.719 (3.89), 7.726 (3.66), 7.734 (3.51), 8.480 (6.50), 8.497 (7.40), 8.665 (10.39), 8.683 (8.45), 8.942 (16.00), 9.563 (1.50), 9.577 (3.14), 9.590 (1.50).

### Example 112

### N-{[7-(2-aminoethyl)-1H-benzimidazol-2-yl]methyl}-8-bromo-2-(morpholin-4-yl)pyrazolo[1,5-a][1,3,5]triazin-4-amine

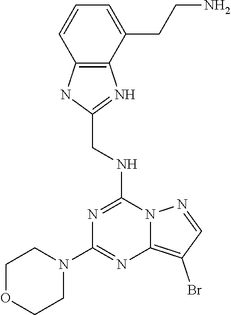 [SMILES: NCCc1cccc2[nH]c(CNc3nc(N4CCOCC4)nc4c(Br)cnn34)nc12]

A solution of N-{[7-(2-aminoethyl)-1H-benzimidazol-2-yl]methyl}-8-bromo-2-(morpholin-4-yl)pyrazolo[1,5-a][1,3,5]triazin-4-amine-N-{[7-(2-aminoethyl)-1H-benzimidazol-2-yl]methyl}-8-chloro-2-(morpholin-4-yl)pyrazolo[1,5-a][1,3,5]triazin-4-amine (1/1) (Intermediate 85, 400 mg, 863 μmol) in hydrobromic acid (30% in acetic acid, 12 mL) was stirred at rt for 2 h. The reaction mixture was evaporated under oil pump at 35° C. to give a residue. The residue was dissolved in methanol, the solution was adjusted to pH 7-8 by sodium carbonate and then filtered, the filtrate was evaporated under reduced pressure to give a residue. The residue was purified by preparative-HPLC (Instrument:Gilson-281; Column: Phenomenex Gemini 150*25 mm*5 um; eluent A: water (0.225% FA), eluent B: acetonitrile; gradient: 0-7.8 min 10-30% B; flow 25 mL/min; temperature: RT; Detector: UV 220/254 nm.) to give 62.8 mg (95% purity, 15% yield) of the title compound as pink solid.

LC-MS (Method D): Rₜ=0.563 min; MS (ESIpos): m/z=472.0 [M+H]⁺.

¹H-NMR (400 MHz, DMSO-d₆): 5 [ppm]=8.40 (s, 1H), 8.03 (s, 1H), 7.34 (d, 2H), 7.08 (t, 1H), 6.99 (d, 1H), 4.89 (s, 2H), 3.50-3.49 (m, 8H), 3.17-3.06 (m, 4H).

### Example 113

### formic acid-N-{[5-(2-aminoethyl)-1H-benzimidazol-2-yl]methyl}-8-bromo-2-(morpholin-4-yl)pyrazolo[1,5-a][1,3,5]triazin-4-amine (1/1)

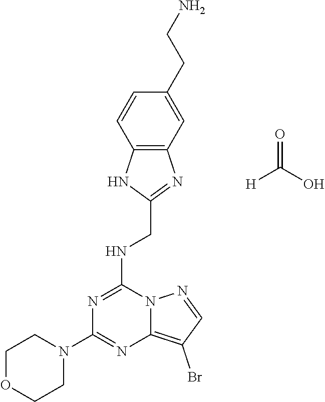 [SMILES: NCCc1ccc2[nH]c(CNc3nc(N4CCOCC4)nc4c(Br)cnn34)nc2c1 ; O=CO]

A solution of tert-butyl {2-[2-({[8-bromo-2-(morpholin-4-yl)pyrazolo[1,5-a][1,3,5]triazin-4-yl]amino}methyl)-1-{[2-(trimethylsilyl)ethoxy]methyl}-1H-benzimidazol-5-yl]ethyl}carbamate-tert-butyl {2-[2-({[8-bromo-2-(morpholin-4-yl)pyrazolo[1,5-a][1,3,5]triazin-4-yl]amino}methyl)-1-{[2-(trimethylsilyl)ethoxy]methyl}-1H-benzimidazol-6-yl]ethyl}carbamate (1:1) (Intermediate 103, 770 mg, 78% purity, 0.855 mmol) in hydrochloric acid in ethyl acetate (10 mL, 4.0 M, 40 mmol) was stirred at rt for 12 h. The reaction mixture was evaporated under reduced pressure to give a residue. The residue was dissolved in methanol, the solution was adjusted to pH 8-9 by sodium hydroxide. The mixture was filtered and the filtrate was evaporated to give a crude product. 100 mg of crude product was used to next step directly. The other was purified by preparative-HPLC [Instrument:Gilson-281; Column: Phenomenex Gemini 150*25 mm*10 um; eluent A: water (0.225% formic acid), eluent B: acetonitrile; gradient: 0-10 min 5-26% B; flow 25 mL/min; temperature: RT; Detector: UV 220/254 nm] to 111 mg (95% purity, 24% yield) of the title compound as a light yellow solid.

LC-MS (Method D): Rₜ=0.549 min; MS (ESIpos): m/z=472.1 [M+H]⁺.

¹H-NMR (400 MHz, DMSO-d₆): 5 [ppm]=8.32 (s, 3H), 8.03 (s, 1H), 7.42 (d, 1H), 7.35 (s, 1H), 7.02 (d, 1H), 4.87 (s, 2H), 3.69-3.59 (m, 8H), 3.56-3.44 (m, 4H), 3.05-2.85 (m, 4H).

### Example 114

### formic acid-N-{[4-(3-aminopropyl)-1H-benzimidazol-2-yl]methyl}-8-bromo-2-(morpholin-4-yl)pyrazolo[1,5-a][1,3,5]triazin-4-amine (1/1)

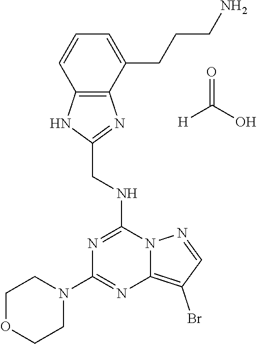

A solution of tert-butyl {3-[2-({[8-bromo-2-(morpholin-4-yl)pyrazolo[1,5-a][1,3,5]triazin-4-yl]amino}methyl)-1-{[2-(trimethylsilyl)ethoxy]methyl}-1H-benzimidazol-4-yl]propyl}carbamate-tert-butyl {3-[2-({[8-bromo-2-(morpholin-4-yl)pyrazolo[1,5-a][1,3,5]triazin-4-yl]amino}methyl)-1-{[2-(trimethylsilyl)ethoxy]methyl}-1H-benzimidazol-7-yl]propyl}carbamate (1:1) (Intermediate 90, 200 mg, 0.140 mmol) in a solvent of hydrochloric acid in ethyl acetate (20 mL, 4.0 M, 80 mmol) was stirred at rt for 16 h. The reaction mixture was concentrated in vacuum and dissolved in methanol. The turbid liquid was adjusted to pH˜7 with sodium carbonate, filtered and purified by preparative HPLC [Gilson-281; Column: Phenomenex Synergi C18 150*25*10 μm; eluent A: water (0.225% formic acid), eluent B: acetonitrile; gradient: 0-9 min, 8%-38% B; flow 25 mL/min; temperature: RT; Detector: UV 220/254 nm] to give 35.6 mg (96% purity, 46% yield) of the title compound as a white solid.

LC-MS (Method D): Rₜ=0.705 min; MS (ESIpos): m/z=486.1 [M+H]⁺.

¹H-NMR (400 MHz, DMSO-d₆): 5 [ppm]=8.45 (s, 1H), 8.04 (s, 1H), 7.30 (d, 1H), 7.06 (d, 1H), 6.96 (d, 1H), 4.89 (s, 2H), 3.63-3.62 (m, 4H), 3.55-3.48- (m, 4H), 2.92 (t, 2H), 2.77 (t, 2H), 1.96-1.89 (m, 2H).

### Example 115

### formic acid-N-{3-[2-({[8-bromo-2-(morpholin-4-yl)pyrazolo[1,5-a][1,3,5]triazin-4-yl]amino}methyl)-1H-benzimidazol-4-yl]propyl}acetamide (1/1)

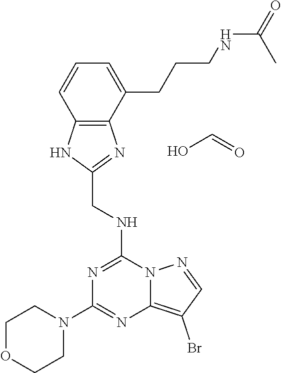 [SMILES: CC(=O)NCCCc1cccc2[nH]c(CNc3nc(N4CCOCC4)nc4c(Br)cnn34)nc12 ; O=CO]

To a solution of formic acid-N-{[4-(3-aminopropyl)-1H-benzimidazol-2-yl]methyl}-8-bromo-2-(morpholin-4-yl)pyrazolo[1,5-a][1,3,5]triazin-4-amine (1/1) (Example 114, 50.0 mg, 98% purity, 92.0 μmol) in N,N-dimethylformamide (1.0 mL) were added pyridine (15 μL, 180 μmol; CAS-RN:[110-86-1]) and acetic anhydride (17 μL, 180 μmol; CAS-RN:[108-24-7]). The reaction mixture was stirred at rt for 16 h. The reaction mixture was poured into water. The solution was extracted with ethyl acetate, concentrated and purified by preparative HPLC to give 37.6 mg (99% purity, 70% yield) of the title compound as a white solid.

### Prep.-Method:

Instrument: Gilson-281; Column: Phenomenex Synergi C18 150*25*10 um; eluent A: water (0.225% formic acid), eluent B: acetonitrile; gradient: 0-9 min, 20%-50% B; flow 25 mL/min; temperature: RT; Detector: UV 220/254 nm

LC-MS (Method D): Rₜ=0.793 min; MS (ESIpos): m/z=528.3 [M+H]⁺.

¹H-NMR (400 MHz, DMSO-d₆): 5 [ppm]=8.16 (s, 1H), 7.81 (s, 1H), 7.34 (d, 1H), 7.15 (t, 1H), 7.06 (d, 1H), 4.99 (s, 2H), 3.68-3.62 (m, 4H), 3.55-3.46 (m, 4H), 3.20 (t, 2H), 2.94 (t, 2H), 1.92-1.87 (m, 5H).

### Example 116

### N-(1H-benzimidazol-2-ylmethyl)-8-bromo-2-(3-ethylmorpholin-4-yl)pyrazolo[1,5-a][1,3,5]triazin-4-amine

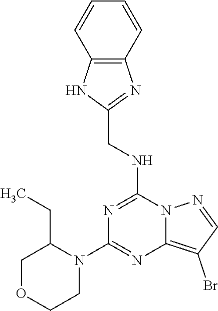 [SMILES: CCC1COCCN1c1nc(NCc2nc3ccccc3[nH]2)n2ncc(Br)c2n1]

N-[(1H-benzimidazol-2-yl)methyl]-8-bromo-2-(methanesulfonyl)pyrazolo[1,5-a][1,3,5]triazin-4-amine (Intermediate 9, 100 mg, 90% purity, 213 μmol) and 3-ethylmorpholine (73.6 mg, 639 μmol, CAS-RN:[55265-24-2]) were provided in acetonitrile (2 mL), N,N-diisopropylethylamine (110 μL, 640 μmol; CAS-RN:[7087-68-5]) was added and the mixture was stirred for 18 h at 70° C. Water was added and the mixture was extracted with ethylacetate. The combined organic layers were filtered over a water repellent filter and concentrated under reduced pressure. The residue was purified by preparative HPLC to give 2.9 mg (3% yield) of the title compound.

### Prep.-Method: BA

Instrument: Waters Autopurificationsystem; Column: XBrigde C18 5μ, 100×30 mm; eluent A: water+0.2 vol % aqueous ammonia (32%); eluent B: acetonitril; gradient: 0.0-0.5 min 38% B (35-70 mL/min), 0.5-5.5 min 38-58% B; flow: 70 mL/min; temperature: 25° C.; DAD scan: 210-400 nm

### Anal.-Method: BA

Instrument: Waters Acquity UPLCMS SingleQuad; Column: Acquity UPLC BEH C18 1.7μ, 50×2.1 mm; eluent A: water+0.2 vol % aqueous ammonia (32%); eluent B: acetonitril; gradient: 0-2.6 min 1-99% B, 2.6-3.0 min 99% B; flow: 0.8 mL/min; temperature: 60° C.; DAD scan: 210-400 nm

Analytical HPLC: Rₜ=1.13 min

LC-MS (Method 2): Rₜ=1.17 min; MS (ESIpos): m/z=457 [M+H]⁺

¹H-NMR (400 MHz, DMSO-d6) δ[ppm]: 0.345 (0.67), 0.800 (0.68), 0.805 (0.46), 0.817 (0.71), 0.824 (0.72), 0.835 (0.48), 0.842 (0.56), 0.853 (0.61), 0.906 (0.60), 1.139 (1.06), 1.234 (2.36), 1.258 (0.48), 1.455 (0.65), 1.907 (1.43), 2.289 (0.40), 2.334 (1.33), 2.338 (0.60), 2.520 (8.09), 2.524 (5.08), 2.542 (1.62), 2.676 (1.37), 2.680 (0.61), 2.968 (0.60), 2.977 (0.72), 3.001 (1.21), 3.007 (1.28), 3.032 (0.97), 3.042 (0.82), 3.650 (0.71), 3.768 (0.53), 4.246 (1.35), 4.279 (1.20), 4.821 (0.96), 4.873 (1.49), 4.885 (1.53), 4.913 (0.70), 4.925 (0.63), 7.091 (0.59), 7.105 (2.27), 7.114 (3.04), 7.121 (3.11), 7.127 (3.37), 7.137 (2.52), 7.151 (0.72), 7.375 (1.70), 7.392 (1.54), 7.514 (1.77), 7.532 (1.63), 8.029 (16.00), 8.240 (0.47), 9.115 (1.44), 12.245 (2.07).

In analogy to the procedure described for Example 116, the following examples were prepared using the appropriate intermediates and amines as starting materials.

TABLE 1
Example 117-240

| | Structure | Synth. from |
|---|---|---|
| | IUPAC-Name | Yield |
| Example | NMR | Analytics |
| 117 | 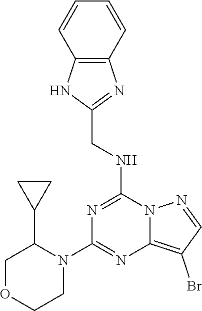 [SMILES: Brc1cnn2c(NCc3nc4ccccc4[nH]3)nc(N3CCOCC3C3CC3)nc12] | Intermediate 9 and CAS- RN: [1260667- 05-7] 3% LC-MS (Method 2): Rₜ = 1.19 min; MS (ESIpos): m/z = 469 [M + H]⁺ |
| | N-(1H-benzimidazol-2-ylmethyl)-8-bromo-2-(3- | |
| | cyclopropylmorpholin-4-yl)pyrazolo[1,5-a][1,3,5]triazin-4-amine | |
| | ¹H-NMR (400 MHz, DMSO-d6) δ [ppm]: 1.235 (0.85), 1.905 (0.89), | |
| | 2.086 (16.00), 2.329 (1.09), 2.334 (0.78), 2.520 (3.37), 2.525 (2.36), | |
| | 2.671 (1.09), 2.676 (0.76), 3.236 (0.83), 3.260 (1.01), 3.814 (0.40), | |
| | 4.327 (0.57), 4.354 (0.49), 4.832 (0.67), 4.844 (0.69), 7.111 (1.24), | |
| | 7.120 (1.41), 7.127 (1.76), 7.134 (1.59), 7.142 (1.25), 7.384 (0.92), | |
| | 7.401 (0.78), 7.405 (0.72), 7.517 (0.87), 7.522 (0.86), 7.538 (0.82), | |
| | 8.022 (7.03), 9.091 (0.68), 12.264 (0.98). | |
| 118 | 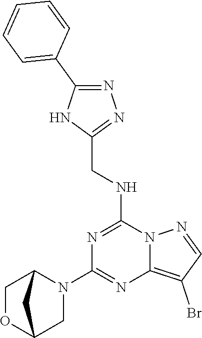 [SMILES: Brc1cnn2c(NCc3nnc(-c4ccccc4)[nH]3)nc(N3C[C@H]4C[C@@H]3CO4)nc12] | Intermediate 24 and CAS- RN: [601515- 79-1] 51% LC-MS (Method 1): Rₜ = 1.00 min; MS (ESIpos): m/z = 468 [M + H]⁺ Specific Optical Rotation (Method O1): +58.7° (c = 10 mg/mL, DMSO) |
| | 8-bromo-2-[(1R,4R)-2-oxa-5-azabicyclo[2.2.1]heptan-5-yl]-N-[(5- | |
| | phenyl-4H-1,2,4-triazol-3-yl)methyl]pyrazolo[1,5-a][1,3,5]triazin- | |
| | 4-amine | |
| | ¹H-NMR (400 MHz, DMSO-d6) δ [ppm]: 1.736 (0.51), 1.760 (0.87), | |
| | 1.817 (1.49), 1.840 (1.14), 1.909 (0.43), 2.077 (1.07), 2.330 (0.90), | |
| | 2.334 (0.64), 2.521 (2.63), 2.526 (1.87), 2.542 (2.90), 2.672 (0.89), | |
| | 2.676 (0.63), 3.401 (2.05), 3.426 (1.59), 3.559 (0.80), 3.576 (0.68), | |
| | 3.676 (0.42), 3.693 (0.50), 3.766 (0.50), 4.588 (3.26), 4.769 (3.37), | |
| | 4.842 (1.08), 4.881 (0.78), 7.419 (1.32), 7.437 (1.88), 7.450 (2.89), | |
| | 7.468 (3.20), 7.485 (1.25), 7.954 (4.19), 7.971 (3.69), 8.007 (16.00), | |
| | 8.021 (0.48). | |
| 119 | 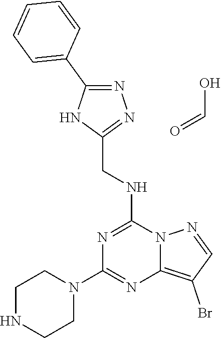 [SMILES: Brc1cnn2c(NCc3nnc(-c4ccccc4)[nH]3)nc(N3CCNCC3)nc12 ; O=CO] | Intermediate 24 and CAS- RN: [110-85-0] 66% LC-MS (Method 1): Rₜ = 0.72 min; MS (ESIpos): m/z = 455 [M + H]⁺ |
| | 8-bromo-N-[(5-phenyl-4H-1,2,4-triazol-3-yl)methyl]-2-piperazin-1- | |
| | yl-pyrazolo[1,5-a][1,3,5]triazin-4-amine formate | |
| | ¹H-NMR (400 MHz, DMSO-d6) δ [ppm]: 1.906 (0.69), 2.077 (8.35), | |
| | 2.325 (0.75), 2.329 (1.05), 2.334 (0.73), 2.521 (4.33), 2.525 (2.80), | |
| | 2.667 (1.08), 2.671 (1.43), 2.676 (1.28), 2.681 (1.02), 2.710 (3.21), | |
| | 3.486 (0.60), 3.655 (3.90), 3.678 (3.75), 4.761 (6.72), 7.405 (0.67), | |
| | 7.409 (0.42), 7.415 (0.43), 7.424 (2.40), 7.430 (0.84), 7.437 (1.57), | |
| | 7.441 (2.83), 7.445 (1.64), 7.454 (4.21), 7.469 (2.13), 7.472 (4.95), | |
| | 7.485 (0.82), 7.489 (1.74), 7.494 (1.23), 7.947 (0.54), 7.953 (4.14), | |
| | 7.957 (5.64), 7.961 (2.46), 7.968 (1.39), 7.973 (4.98), 7.978 (3.82), | |
| | 8.010 (16.00), 8.024 (0.94), 8.226 (13.47). | |
| 120 | 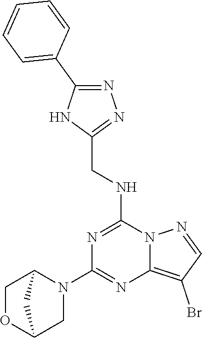 [SMILES: Brc1cnn2c(NCc3nnc(-c4ccccc4)[nH]3)nc(N3C[C@@H]4C[C@H]3CO4)nc12] | Intermediate 24 and CAS- RN: [909186- 56-7] 52% LC-MS (Method 1): Rₜ = 0.99 min; MS (ESIpos): m/z = 468 [M + H]⁺ Specific Optical Rotation (Method O1): −57.7° (c = 10 mg/mL, DMSO) |
| | 8-bromo-2-[(1S,4S)-2-oxa-5-azabicyclo[2.2.1]heptan-5-yl]-N-[(5- | |
| | phenyl-4H-1,2,4-triazol-3-yl)methyl]pyrazolo[1,5-a][1,3,5]triazin- | |
| | 4-amine | |
| | ¹H-NMR (400 MHz, DMSO-d6) δ [ppm]: 1.736 (0.50), 1.759 (0.88), | |
| | 1.817 (1.49), 1.838 (1.16), 1.909 (0.64), 2.077 (2.71), 2.521 (3.61), | |
| | 2.525 (2.29), 2.542 (1.00), 3.400 (2.33), 3.425 (1.79), 3.559 (0.80), | |
| | 3.575 (0.70), 3.676 (0.43), 3.693 (0.54), 3.766 (0.51), 4.588 (3.38), | |
| | 4.767 (2.64), 4.842 (1.04), 4.881 (0.83), 7.418 (1.18), 7.437 (1.75), | |
| | 7.449 (2.74), 7.467 (3.08), 7.484 (1.25), 7.954 (4.24), 7.971 (3.77), | |
| | 8.006 (16.00), 8.020 (0.68), 8.207 (1.01), 9.091 (0.55). | |
| 121 | 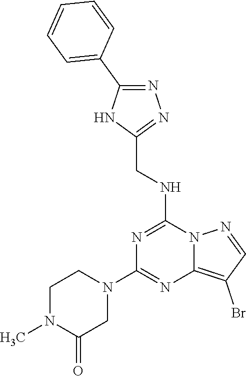 [SMILES: CN1CCN(c2nc(NCc3nnc(-c4ccccc4)[nH]3)n3ncc(Br)c3n2)CC1=O] | Intermediate 24 and CAS- RN: [109384- 27-2] 45% LC-MS (Method 1): Rₜ = 0.96 min; MS (ESIneg): m/z = 481 [M − H]⁻ |
| | 4-(8-bromo-4-{[(5-phenyl-4H-1,2,4-triazol-3- | |
| | yl)methyl]amino}pyrazolo[1,5-a][1,3,5]triazin-2-yl)-1- | |
| | methylpiperazin-2-one | |
| | ¹H-NMR (400 MHz, DMSO-d6) δ [ppm]: 2.077 (0.47), 2.335 (0.70), | |
| | 2.521 (4.04), 2.526 (2.52), 2.543 (11.06), 2.673 (0.98), 2.677 (0.73), | |
| | 2.830 (14.24), 3.932 (3.85), 4.227 (8.90), 4.799 (2.45), 7.454 (3.01), | |
| | 7.472 (3.15), 7.963 (6.36), 7.980 (5.60), 7.984 (4.67), 8.053 (16.00), | |
| | 8.067 (0.68), 9.202 (0.63). | |
| 122 | 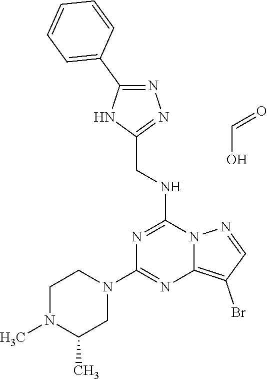 [SMILES: C[C@H]1CN(c2nc(NCc3nnc(-c4ccccc4)[nH]3)n3ncc(Br)c3n2)CCN1C ; O=CO] | Intermediate 24 and CAS- RN: [485841- 50-7] 43% LC-MS (Method 2): Rₜ = 0.93 min; MS (ESIpos): m/z = 483 [M + H]⁺ Specific Optical Rotation (Method O1): −11.0° (c = 10 mg/mL, DMSO) |
| | 8-bromo-2-[(3S)-3,4-dimethylpiperazin-1-yl]-N-[(5-phenyl-4H- | |
| | 1,2,4-triazol-3-yl)methyl]pyrazolo[1,5-a][1,3,5]triazin-4-amine | |
| | formate | |
| | ¹H-NMR (400 MHz, DMSO-d6) δ [ppm]: −0.008 (1.41), 0.008 (1.30), | |
| | 0.930 (1.17), 1.879 (0.48), 1.909 (0.54), 1.976 (0.61), 2.077 (0.41), | |
| | 2.131 (16.00), 2.325 (0.95), 2.329 (1.23), 2.334 (0.91), 2.339 (0.43), | |
| | 2.521 (4.74), 2.525 (2.90), 2.542 (2.14), 2.584 (0.99), 2.609 (1.10), | |
| | 2.616 (1.19), 2.641 (1.02), 2.667 (1.47), 2.672 (1.88), 2.676 (1.54), | |
| | 2.994 (0.65), 3.341 (4.61), 4.340 (0.91), 4.371 (0.84), 4.750 (2.34), | |
| | 4.762 (2.25), 7.421 (1.15), 7.439 (1.64), 7.451 (2.59), 7.469 (2.90), | |
| | 7.486 (1.12), 7.951 (3.85), 7.954 (4.86), 7.971 (4.48), 7.975 (3.50), | |
| | 8.005 (14.05), 8.019 (0.41), 8.149 (11.83), 9.079 (0.93). | |
| 123 | 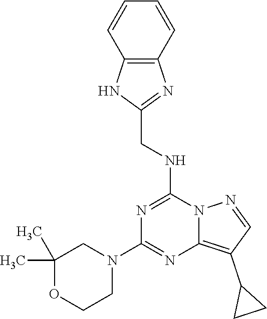 [SMILES: CC1(C)CN(c2nc(NCc3nc4ccccc4[nH]3)n3ncc(C4CC4)c3n2)CCO1] | Intermediate 57 and CAS- RN: [167946- 94-3] 13% LC-MS (Method 2): Rₜ = 1.16 min; MS (ESIpos): m/z = 419 [M + H]⁺ |
| | N-[(1H-benzimidazol-2-yl)methyl]-8-cyclopropyl-2-(2,2- | |
| | dimethylmorpholin-4-yl)pyrazolo[1,5-a][1,3,5]triazin-4-amine | |
| | ¹H-NMR (400 MHz, DMSO-d6) δ [ppm]: 0.701 (1.72), 0.706 (1.32), | |
| | 0.714 (4.72), 0.719 (5.74), 0.726 (6.82), 0.731 (5.73), 0.740 (2.94), | |
| | 0.753 (1.24), 0.759 (1.52), 0.766 (3.29), 0.774 (6.16), 0.779 (4.05), | |
| | 0.787 (4.37), 0.791 (3.05), 0.795 (6.16), 0.800 (4.21), 0.806 (2.01), | |
| | 0.812 (2.60), 0.854 (2.82), 0.871 (2.84), 1.234 (1.72), 1.703 (0.92), | |
| | 1.716 (1.74), 1.723 (1.77), 1.737 (2.75), 1.745 (1.07), 1.749 (1.61), | |
| | 1.758 (1.53), 1.770 (0.72), 2.329 (2.61), 2.334 (1.88), 2.338 (0.81), | |
| | 2.520 (8.36), 2.525 (5.86), 2.542 (1.57), 2.671 (2.61), 2.676 (1.83), | |
| | 2.680 (0.79), 3.370 (3.41), 3.491 (2.72), 3.569 (3.23), 4.798 (3.78), | |
| | 7.111 (3.14), 7.123 (3.25), 7.389 (1.01), 7.509 (1.05), 7.694 (16.00), | |
| | 8.553 (0.49), 8.860 (1.59), 12.269 (1.97). | |
| 124 | 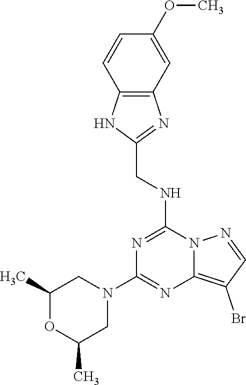 [SMILES: COc1ccc2[nH]c(CNc3nc(N4C[C@@H](C)O[C@@H](C)C4)nc4c(Br)cnn34)nc2c1] | Intermediate 105 and CAS- RN: [6485-55- 8] 40% LC-MS (Method 2): Rₜ = 1.14 min; MS (ESIpos): m/z = 487 [M + H]⁺ |
| | 8-bromo-2-[(2R, 6S)-2, 6-dimethylmorpholin-4-yl]-N-[(5-methoxy- | |
| | 1H-benzimidazol-2-yl)methyl]pyrazolo[1,5-a][1,3,5]triazin-4- | |
| | amine | |
| | ¹H-NMR (400 MHz, DMSO-d6) δ [ppm]: 1.047 (2.76), 2.325 (0.97), | |
| | 2.329 (1.28), 2.334 (1.06), 2.408 (1.00), 2.443 (0.90), 2.525 (4.71), | |
| | 2.542 (1.07), 2.667 (0.75), 2.671 (1.01), 2.676 (0.76), 3.747 (16.00), | |
| | 4.378 (0.87), 4.823 (3.27), 6.738 (0.99), 6.759 (1.68), 6.780 (0.76), | |
| | 6.884 (1.66), 7.066 (1.14), 7.259 (0.84), 7.280 (0.82), 7.394 (1.32), | |
| | 7.415 (1.24), 8.028 (13.29), 9.097 (1.19), 12.087 (1.21), 12.130 | |
| | (0.89). | |
| 125 | 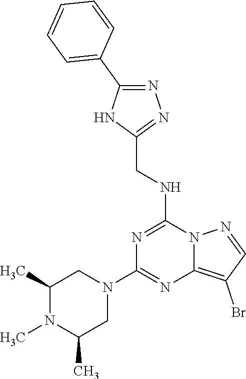 [SMILES: C[C@@H]1CN(c2nc(NCc3nnc(-c4ccccc4)[nH]3)n3ncc(Br)c3n2)C[C@H](C)N1C] | Intermediate 24 and CAS- RN: [147539- 61-5] 54% LC-MS (Method 2): Rₜ = 0.97 min; MS (ESIneg): m/z = 495 [M − H]⁻ |
| | 8-bromo-N-[(5-phenyl-4H-1,2,4-triazol-3-yl)methyl]-2-[(3R,5S)- | |
| | 3,4,5-trimethylpiperazin-1-yl]pyrazolo[1,5-a][1,3,5]triazin-4- | |
| | amine | |
| | ¹H-NMR (400 MHz, DMSO-d6) δ [ppm]: 0.973 (1.70), 1.909 (0.48), | |
| | 1.938 (0.48), 2.106 (16.00), 2.334 (0.61), 2.521 (3.60), 2.526 (2.56), | |
| | 2.542 (2.10), 2.672 (0.85), 2.676 (0.62), 4.354 (0.68), 4.745 (2.08), | |
| | 4.758 (2.13), 7.417 (1.03), 7.435 (1.45), 7.447 (2.36), 7.466 (2.66), | |
| | 7.482 (0.99), 7.952 (3.35), 7.955 (4.34), 7.958 (2.13), 7.967 (1.22), | |
| | 7.972 (3.86), 7.976 (3.16), 8.007 (12.60), 8.021 (0.48), 8.032 (0.53), | |
| | 8.151 (10.07), 9.089 (0.86). | |
| 126 | 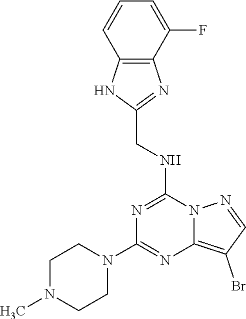 [SMILES: CN1CCN(c2nc(NCc3nc4c(F)cccc4[nH]3)n3ncc(Br)c3n2)CC1] | Intermediate 64 and CAS- RN: [109-01-3] 66% LC-MS (Method 2): Rₜ = 1.06 min; MS (ESIpos): m/z = 460 [M + H]⁺ |
| | 8-bromo-N-[(4-fluoro-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: −0.149 (0.42), 0.146 (0.41), | |
| | 0.801 (1.30), 0.806 (0.55), 0.817 (1.34), 0.824 (1.36), 0.842 (0.66), | |
| | 0.888 (0.73), 0.907 (1.57), 0.925 (0.82), 1.038 (7.40), 1.055 (16.00), | |
| | 1.073 (8.28), 1.236 (1.39), 1.909 (0.76), 2.132 (9.53), 2.196 (3.48), | |
| | 2.213 (2.20), 2.339 (1.04), 2.361 (0.59), 2.376 (0.54), 2.521 (9.88), | |
| | 2.525 (6.28), 2.681 (0.78), 3.407 (0.68), 3.420 (0.79), 3.425 (1.82), | |
| | 3.437 (1.87), 3.442 (1.82), 3.455 (1.81), 3.460 (0.75), 3.472 (0.68), | |
| | 3.622 (3.69), 3.735 (0.42), 4.346 (0.82), 4.358 (1.57), 4.370 (0.82), | |
| | 4.866 (6.30), 4.880 (6.35), 6.917 (1.74), 6.935 (2.24), 6.945 (1.84), | |
| | 6.963 (2.13), 7.005 (0.56), 7.033 (0.52), 7.100 (1.69), 7.112 (1.53), | |
| | 7.120 (3.31), 7.132 (2.90), 7.140 (2.00), 7.152 (1.67), 7.236 (4.63), | |
| | 7.255 (3.33), 7.379 (0.93), 7.399 (0.79), 7.945 (1.06), 8.021 (2.84), | |
| | 8.028 (14.08), 8.043 (0.58), 9.094 (0.56), 9.133 (1.13), 9.148 (2.35), | |
| | 9.162 (1.16), 12.580 (2.41), 12.888 (0.75). | |
| 127 | 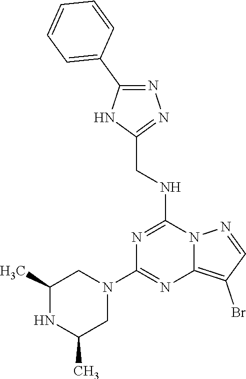 [SMILES: C[C@@H]1CN(c2nc(NCc3nnc(-c4ccccc4)[nH]3)n3ncc(Br)c3n2)C[C@H](C)N1] | Intermediate 24 and CAS- RN: [108-49-6] 34% LC-MS (Method 2): Rₜ = 0.85 min; MS (ESIpos): m/z = 483 [M + H]⁺ |
| | 8-bromo-2-[(3R,5S)-3,5-dimethylpiperazin-1-yl]-N-[(5-phenyl-4H- | |
| | 1,2,4-triazol-3-yl)methyl]pyrazolo[1,5-a][1,3,5]triazin-4-amine | |
| | ¹H-NMR (500 MHz, DMSO-d6) δ [ppm]: 0.954 (14.05), 0.967 | |
| | (14.15), 2.249 (2.63), 2.271 (3.46), 2.274 (3.19), 2.295 (3.00), 2.357 | |
| | (0.53), 2.361 (0.75), 2.365 (0.55), 2.513 (2.97), 2.517 (2.45), 2.521 | |
| | (2.10), 2.587 (1.01), 2.592 (1.22), 2.599 (1.29), 2.606 (1.58), 2.619 | |
| | (1.13), 2.626 (1.04), 2.631 (0.96), 2.635 (1.08), 2.638 (0.84), 3.068 | |
| | (16.00), 4.461 (2.10), 4.465 (2.09), 4.486 (2.14), 4.783 (11.25), | |
| | 7.397 (0.65), 7.400 (0.45), 7.406 (0.50), 7.412 (2.62), 7.416 (0.79), | |
| | 7.423 (1.42), 7.426 (2.67), 7.429 (1.35), 7.443 (4.15), 7.445 (1.91), | |
| | 7.454 (2.60), 7.457 (5.40), 7.468 (0.89), 7.471 (2.20), 7.474 (1.33), | |
| | 7.908 (11.08), 7.915 (0.82), 7.961 (0.52), 7.966 (3.98), 7.968 (5.35), | |
| | 7.971 (2.49), 7.978 (1.37), 7.982 (5.57), 7.985 (4.03), 8.231 (2.78). | |
| 128 | 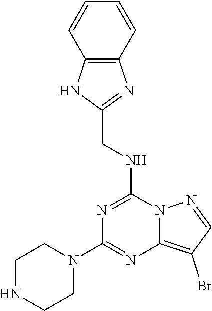 [SMILES: Brc1cnn2c(NCc3nc4ccccc4[nH]3)nc(N3CCNCC3)nc12] | Intermediate 9 and CAS- RN: [110-85-0] 48% LC-MS (Method 2): Rₜ = 0.90 min; MS (ESIpos): m/z = 428 [M + H]⁺ |
| | N-[(1H-benzimidazol-2-yl)methyl]-8-bromo-2-(piperazin-1- | |
| | yl)pyrazolo[1,5-a][1,3,5]triazin-4-amine | |
| | ¹H-NMR (400 MHz, DMSO-d6) δ [ppm]: 2.325 (0.60), 2.329 (0.82), | |
| | 2.334 (0.60), 2.521 (3.90), 2.525 (2.87), 2.542 (4.33), 2.564 (2.59), | |
| | 2.662 (0.48), 2.667 (0.79), 2.672 (0.99), 2.676 (0.76), 3.565 (4.32), | |
| | 4.857 (11.38), 7.123 (2.88), 7.135 (3.08), 7.410 (0.91), 7.527 (0.92), | |
| | 8.008 (16.00), 8.022 (0.59), 8.365 (0.59), 12.261 (0.88). | |
| 129 | 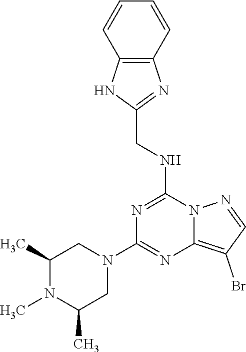 [SMILES: C[C@@H]1CN(c2nc(NCc3nc4ccccc4[nH]3)n3ncc(Br)c3n2)C[C@H](C)N1C] | Intermediate 9 and CAS- RN: [147539- 61-5] 70% LC-MS (Method 2): Rt = 1.11 min; MS (ESIpos): m/z = 470 [M + H]⁺ |
| | N-(1H-benzimidazol-2-ylmethyl)-8-bromo-2-[(3R,5S)-3,4,5- | |
| | trimethylpiperazin-1-yl]pyrazolo[1,5-a][1,3,5]triazin-4-amine | |
| | ¹H-NMR (400 MHz, DMSO-d6) δ [ppm]: 0.855 (0.82), 0.952 (0.84), | |
| | 2.060 (16.00), 2.329 (0.70), 2.334 (0.54), 2.520 (5.18), 2.525 (3.43), | |
| | 2.581 (0.41), 2.671 (0.69), 2.676 (0.51), 4.838 (2.55), 4.851 (2.56), | |
| | 7.094 (0.44), 7.108 (1.54), 7.112 (1.31), 7.119 (1.84), 7.125 (2.37), | |
| | 7.130 (2.04), 7.137 (1.44), 7.142 (1.78), 7.156 (0.54), 7.380 (1.40), | |
| | 7.397 (1.26), 7.401 (1.16), 7.515 (1.25), 7.519 (1.32), 7.536 (1.25), | |
| | 8.019 (10.83), 9.092 (0.67), 9.105 (1.35), 9.119 (0.70), 12.272 | |
| | (1.87). | |
| 130 | 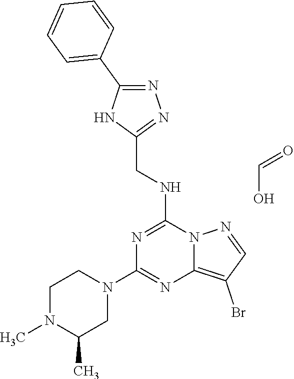 | Intermediate 24 and CAS- RN: [1152110- 23-0] 30% LC-MS (Method 1): Rₜ = 0.76 min; MS (ESIneg): m/z = 481 [M − H]⁻ Specific Optical Rotation (Method O1): +13.9° (c = 10 mg/mL, DMSO) |
| | 8-bromo-2-[(3R)-3,4-dimethylpiperazin-1-yl]-N-[(5-phenyl-4H- | |
| | 1,2,4-triazol-3-yl)methyl]pyrazolo[1,5-a][1,3,5]triazin-4-amine | |
| | formate | |
| | ¹H-NMR (400 MHz, DMSO-d6) δ [ppm]: 0.928 (1.14), 1.869 (0.45), | |
| | 1.967 (0.59), 2.077 (0.58), 2.127 (16.00), 2.325 (0.66), 2.329 (0.89), | |
| | 2.334 (0.65), 2.521 (3.19), 2.525 (2.02), 2.542 (2.65), 2.581 (1.04), | |
| | 2.606 (1.14), 2.613 (1.21), 2.639 (1.07), 2.667 (1.22), 2.672 (1.45), | |
| | 2.676 (1.19), 2.991 (0.63), 4.340 (0.92), 4.371 (0.84), 4.750 (2.64), | |
| | 4.762 (2.67), 7.403 (0.42), 7.420 (1.39), 7.438 (1.85), 7.450 (3.05), | |
| | 7.469 (3.48), 7.486 (1.27), 7.951 (3.93), 7.955 (5.14), 7.958 (2.46), | |
| | 7.966 (1.51), 7.971 (4.69), 7.975 (3.61), 8.005 (13.62), 8.019 (0.76), | |
| | 8.028 (0.41), 8.158 (7.35), 9.077 (1.06). | |
| 131 | 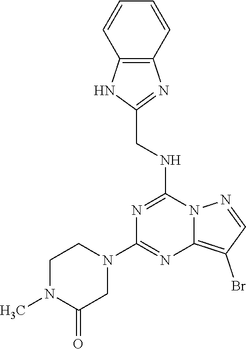 [SMILES: CN1CCN(c2nc(NCc3nc4ccccc4[nH]3)n3ncc(Br)c3n2)CC1=O] | Intermediate 9 and CAS- RN: [109384- 27-2] 50% LC-MS (Method 2): Rₜ = 0.93 min; MS (ESIpos): m/z = 456 [M + H]⁺ |
| | 4-(4-{[(1H-benzimidazol-2-yl)methyl]amino}-8- | |
| | bromopyrazolo[1,5-a][1,3,5]triazin-2-yl)-1-methylpiperazin-2-one | |
| | ¹H-NMR (400 MHz, DMSO-d6) δ [ppm]: 2.077 (0.98), 2.334 (0.83), | |
| | 2.520 (4.52), 2.525 (2.91), 2.542 (0.79), 2.676 (0.87), 2.799 (6.17), | |
| | 3.881 (1.76), 4.199 (5.03), 4.894 (3.66), 4.904 (3.68), 7.106 (0.69), | |
| | 7.120 (2.45), 7.124 (2.04), 7.130 (2.80), 7.137 (3.84), 7.142 (3.05), | |
| | 7.149 (2.12), 7.153 (2.75), 7.167 (0.78), 7.399 (2.15), 7.405 (1.35), | |
| | 7.416 (1.94), 7.420 (1.71), 7.533 (1.92), 7.538 (2.00), 7.555 (1.88), | |
| | 8.068 (16.00), 8.083 (0.50), 9.223 (1.22), 12.263 (2.51). | |
| 132 | 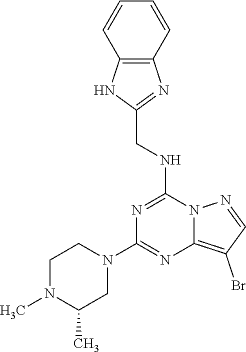 [SMILES: C[C@H]1CN(c2nc(NCc3nc4ccccc4[nH]3)n3ncc(Br)c3n2)CCN1C] | Intermediate 9 and CAS- RN: [485841- 50-7] 22% LC-MS (Method 2): Rₜ = 1.07 min; MS (ESIpos): m/z = 456 [M + H]⁺ |
| | N-[(1H-benzimidazol-2-yl)methyl]-8-bromo-2-[(3S)-3,4- | |
| | dimethylpiperazin-1-yl]pyrazolo[1,5-a][1,3,5]triazin-4-amine | |
| | ¹H-NMR (400 MHz, DMSO-d6) δ [ppm]: −0.149 (0.46), 0.146 (0.50), | |
| | 0.856 (0.72), 1.902 (0.57), 2.097 (16.00), 2.334 (2.88), 2.338 (1.27), | |
| | 2.520 (15.98), 2.525 (10.23), 2.542 (1.80), 2.676 (3.03), 4.288 | |
| | (0.95), 4.320 (0.89), 4.847 (3.59), 4.860 (3.50), 7.097 (0.73), 7.111 | |
| | (2.43), 7.115 (2.05), 7.122 (2.56), 7.127 (3.95), 7.134 (2.89), 7.140 | |
| | (2.20), 7.144 (2.55), 7.158 (0.79), 7.385 (2.05), 7.401 (1.95), 7.406 | |
| | (1.61), 7.519 (1.87), 7.524 (1.95), 7.541 (1.77), 8.018 (15.43), 9.078 | |
| | (1.01), 9.092 (2.02), 9.106 (1.01), 12.262 (2.68). | |
| 133 | 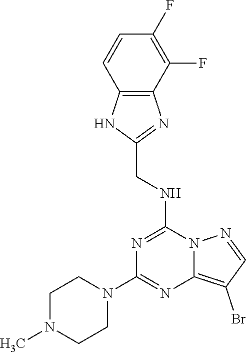 [SMILES: CN1CCN(c2nc(NCc3nc4c(F)c(F)ccc4[nH]3)n3ncc(Br)c3n2)CC1] | Intermediate 26 and CAS- RN: [109-01-3] 71% LC-MS (Method 2): Rₜ = 1.03 min; MS (ESIpos): m/z = 478 [M + H]⁺ |
| | 8-bromo-N-[(4,5-difluoro-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: 0.907 (0.45), 1.234 (0.97), | |
| | 2.065 (0.45), 2.087 (0.51), 2.113 (16.00), 2.158 (1.83), 2.166 (1.54), | |
| | 2.188 (1.83), 2.200 (1.11), 2.320 (0.49), 2.325 (1.02), 2.329 (1.40), | |
| | 2.334 (1.01), 2.339 (0.51), 2.521 (4.54), 2.525 (3.08), 2.662 (0.42), | |
| | 2.667 (0.93), 2.671 (1.31), 2.676 (0.92), 2.680 (0.41), 3.409 (0.81), | |
| | 3.609 (2.57), 4.859 (3.11), 4.870 (3.12), 7.179 (0.96), 7.196 (1.34), | |
| | 7.206 (1.81), 7.218 (1.69), 8.026 (10.06), 9.155 (0.73), 12.690 | |
| | (0.59). | |
| 134 | 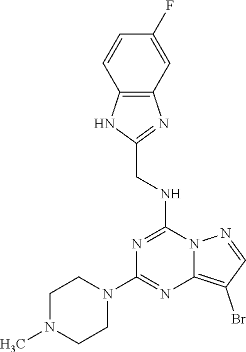 [SMILES: CN1CCN(c2nc(NCc3nc4cc(F)ccc4[nH]3)n3ncc(Br)c3n2)CC1] | Intermediate 66 and CAS- RN: [109-01-3] 23% LC-MS (Method 2): Rt = 1.03 min; MS (ESIneg): m/z = 458 [M − H]⁻ |
| | 8-bromo-N-[(5-fluoro-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: 1.234 (0.46), 2.119 (16.00), | |
| | 2.175 (1.62), 2.334 (0.75), 2.520 (4.29), 2.525 (2.80), 2.676 (0.68), | |
| | 3.619 (2.76), 4.848 (6.41), 6.964 (0.73), 6.969 (0.82), 6.988 (1.19), | |
| | 6.992 (1.30), 7.010 (0.84), 7.016 (0.89), 7.272 (0.46), 7.456 (0.45), | |
| | 8.020 (12.26), 8.034 (1.71). | |
| 135 | 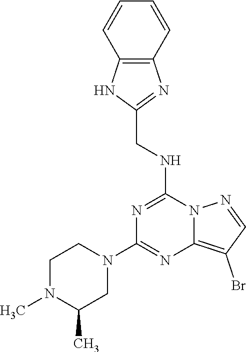 [SMILES: C[C@@H]1CN(c2nc(NCc3nc4ccccc4[nH]3)n3ncc(Br)c3n2)CCN1C] | Intermediate 9 and CAS- RN1152110- 23-0] 16% LC-MS (Method 2): Rₜ = 1.06 min; MS (ESIpos): m/z = 456 [M + H]⁺ |
| | N-[(1H-benzimidazol-2-yl)methyl]-8-bromo-2-[(3R)-3,4- | |
| | dimethylpiperazin-1-yl]pyrazolo[1,5-a][1,3,5]triazin-4-amine | |
| | ¹H-NMR (400 MHz, DMSO-d6) δ [ppm]: 0.855 (0.66), 1.894 (0.43), | |
| | 2.099 (15.34), 2.325 (0.91), 2.329 (1.31), 2.334 (0.93), 2.338 (0.41), | |
| | 2.520 (4.81), 2.525 (3.60), 2.542 (2.02), 2.662 (0.68), 2.667 (1.17), | |
| | 2.671 (1.54), 2.676 (1.12), 2.948 (0.48), 4.289 (0.97), 4.321 (0.88), | |
| | 4.847 (3.92), 4.861 (3.86), 7.093 (0.52), 7.097 (0.84), 7.111 (2.63), | |
| | 7.115 (2.43), 7.122 (2.80), 7.128 (4.90), 7.134 (3.08), 7.140 (2.53), | |
| | 7.144 (2.97), 7.158 (1.00), 7.162 (0.60), 7.385 (2.34), 7.391 (1.46), | |
| | 7.402 (2.27), 7.406 (1.84), 7.519 (2.08), 7.524 (2.16), 7.535 (1.22), | |
| | 7.541 (2.04), 8.018 (16.00), 9.079 (1.15), 9.093 (2.48), 9.107 (1.17), | |
| | 12.259 (3.04). | |
| 136 | 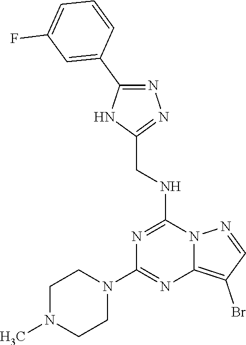 [SMILES: CN1CCN(c2nc(NCc3nnc(-c4cccc(F)c4)[nH]3)n3ncc(Br)c3n2)CC1] | Intermediate 109 and CAS- RN: [109-01-3] 35% LC-MS (Method 2): Rₜ = 0.77 min; MS (ESIpos): m/z = 487 [M + H]⁺ |
| | 8-bromo-N-{[5-(3-fluorophenyl)-4H-1,2,4-triazol-3-yl]methyl}-2- | |
| | (4-methylpiperazin-1-yl)pyrazolo[1,5-a][1,3,5]triazin-4-amine | |
| | ¹H-NMR (400 MHz, DMSO-d6) δ [ppm]: 0.940 (0.73), 0.957 (0.72), | |
| | 2.136 (16.00), 2.188 (0.54), 2.234 (2.91), 2.334 (0.87), 2.521 (3.79), | |
| | 2.526 (2.51), 2.542 (1.65), 3.662 (3.99), 4.777 (2.96), 7.242 (0.60), | |
| | 7.247 (0.67), 7.263 (1.24), 7.269 (1.32), 7.285 (0.71), 7.290 (0.75), | |
| | 7.493 (0.90), 7.508 (1.08), 7.513 (1.62), 7.528 (1.60), 7.533 (1.04), | |
| | 7.548 (0.85), 7.676 (1.18), 7.680 (1.36), 7.682 (1.42), 7.686 (1.25), | |
| | 7.701 (1.19), 7.705 (1.39), 7.707 (1.32), 7.711 (1.22), 7.802 (1.92), | |
| | 7.804 (2.61), 7.807 (1.84), 7.821 (1.71), 7.824 (2.27), 7.827 (1.56), | |
| | 8.009 (14.55), 9.099 (0.86). | |
| 137 | 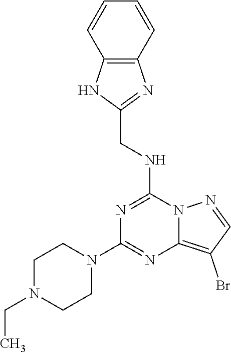 [SMILES: CCN1CCN(c2nc(NCc3nc4ccccc4[nH]3)n3ncc(Br)c3n2)CC1] | Intermediate 9 and CAS- RN: [5308-25- 8] 75% LC-MS (Method 2): Rₜ = 1.03 min; MS (ESIpos): m/z = 456 [M + H]⁺ |
| | N-[(1H-benzimidazol-2-yl)methyl]-8-bromo-2-(4-ethylpiperazin-1- | |
| | yl)pyrazolo[1,5-a][1,3,5]triazin-4-amine | |
| | ¹H-NMR (400 MHz,DMSO-d6) δ [ppm]: 0.935 (5.86), 0.953 (13.76), | |
| | 0.971 (6.29), 2.222 (3.24), 2.239 (6.55), 2.257 (5.99), 2.275 (1.95), | |
| | 2.320 (0.40), 2.325 (0.67), 2.330 (0.86), 2.334 (0.66), 2.521 (3.38), | |
| | 2.525 (2.10), 2.542 (1.60), 2.667 (0.52), 2.672 (0.73), 2.676 (0.54), | |
| | 3.624 (3.80), 4.860 (11.29), 7.106 (0.84), 7.116 (6.00), 7.124 (5.39), | |
| | 7.131 (5.64), 7.139 (6.40), 7.149 (1.04), 7.457 (2.70), 7.465 (2.67), | |
| | 7.472 (2.60), 7.480 (2.39), 8.015 (16.00), 8.029 (0.47). | |
| 138 | 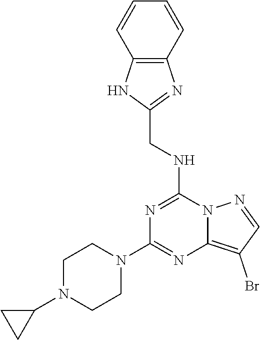 [SMILES: Brc1cnn2c(NCc3nc4ccccc4[nH]3)nc(N3CCN(C4CC4)CC3)nc12] | Intermediate 9 and CAS- RN: [20327- 23-5] 65% LC-MS (Method 2): Rₜ = 1.13 min; MS (ESIpos): m/z = 468 [M + H]⁺ |
| | N-[(1H-benzimidazol-2-yl)methyl]-8-bromo-2-(4- | |
| | cyclopropylpiperazin-1-yl)pyrazolo[1,5-a][1,3,5]triazin-4-amine | |
| | ¹H-NMR (400 MHz, DMSO-d6) δ [ppm]: −0.003 (11.37), 0.275 (1.35), | |
| | 0.292 (6.21), 0.299 (5.92), 0.308 (2.03), 0.337 (0.52), 0.349 (0.46), | |
| | 0.373 (1.77), 0.383 (4.64), 0.388 (4.92), 0.399 (5.36), 0.415 (1.18), | |
| | 1.511 (0.73), 1.520 (1.45), 1.527 (1.99), 1.536 (2.71), 1.544 (1.97), | |
| | 1.552 (1.43), 2.327 (1.43), 2.332 (1.20), 2.414 (3.48), 2.539 (2.18), | |
| | 2.665 (0.88), 2.669 (1.14), 2.673 (0.87), 3.592 (6.15), 4.865 (6.05), | |
| | 4.872 (6.05), 7.114 (2.86), 7.123 (4.16), 7.130 (4.08), 7.136 (4.55), | |
| | 7.145 (3.25), 7.394 (2.33), 7.411 (2.14), 7.528 (2.33), 7.546 (2.12), | |
| | 8.017 (16.00), 8.031 (0.59), 9.081 (2.13), 12.258 (3.34). | |
| 139 | 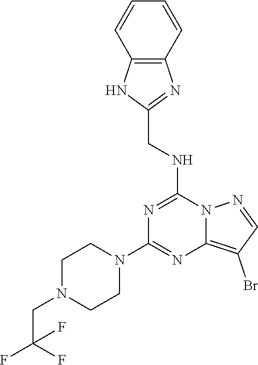 [SMILES: FC(F)(F)CN1CCN(c2nc(NCc3nc4ccccc4[nH]3)n3ncc(Br)c3n2)CC1] | Intermediate 9 and CAS- RN: [13349- 90-1] 64% LC-MS (Method 2): Rₜ = 1.20 min; MS (ESIpos): m/z = 510 [M + H]⁺ |
| | N-[(1H-benzimidazol-2-yl)methyl]-8-bromo-2-[4-(2,2,2- | |
| | trifluoroethyl)piperazin-1-yl]pyrazolo[1,5-a][1,3,5]triazin-4-amine | |
| | ¹H-NMR (400 MHz, DMSO-d6) δ [ppm]: 0.000 (6.66), 2.325 (1.16), | |
| | 2.330 (1.57), 2.334 (1.16), 2.389 (1.80), 2.525 (7.21), 2.542 (2.77), | |
| | 2.667 (1.20), 2.672 (1.62), 2.676 (1.20), 3.093 (1.80), 3.118 (5.23), | |
| | 3.144 (4.99), 3.170 (1.57), 3.386 (0.55), 3.650 (6.29), 3.873 (1.34), | |
| | 4.863 (5.50), 4.871 (5.55), 4.987 (0.55), 7.101 (0.79), 7.115 (2.82), | |
| | 7.125 (3.75), 7.131 (4.39), 7.137 (4.16), 7.147 (3.38), 7.161 (0.97), | |
| | 7.394 (2.64), 7.411 (2.31), 7.529 (2.45), 7.546 (2.31), 8.029 (16.00), | |
| | 8.043 (0.55), 9.119 (1.90), 12.258 (3.38). | |
| 140 | 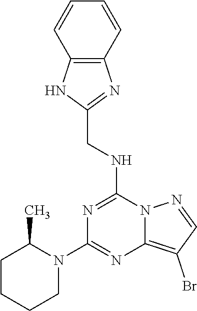 [SMILES: C[C@@H]1CCCCN1c1nc(NCc2nc3ccccc3[nH]2)n2ncc(Br)c2n1] | Intermediate 9 and CAS- RN: [1722-95- 8] 5% LC-MS (Method 2): Rₜ = 1.27 min; MS (ESIpos): m/z = 441 [M + H]⁺ |
| | N-[(1H-benzimidazol-2-yl)methyl]-8-bromo-2-[(2R)-2- | |
| | methylpiperidin-1-yl]pyrazolo[1,5-a][1,3,5]triazin-4-amine | |
| | ¹H-NMR (400 MHz, DMSO-d6) δ [ppm]: −0.003 (6.08), 0.859 (1.05), | |
| | 0.902 (1.63), 0.967 (2.85), 1.107 (16.00), 1.144 (1.92), 1.197 (0.72), | |
| | 1.232 (0.80), 1.388 (0.60), 1.449 (2.77), 1.474 (2.10), 1.557 (1.39), | |
| | 2.749 (0.79), 2.777 (1.45), 2.810 (0.78), 4.194 (1.33), 4.440 (0.98), | |
| | 4.471 (0.95), 4.840 (5.20), 7.112 (2.85), 7.123 (3.03), 7.396 (0.91), | |
| | 7.520 (0.92), 7.993 (9.53), 9.013 (0.95), 12.253 (1.20). | |
| 141 | 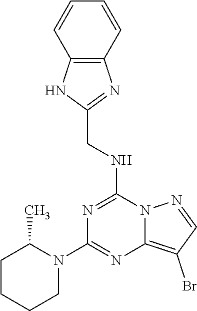 [SMILES: C[C@H]1CCCCN1c1nc(NCc2nc3ccccc3[nH]2)n2ncc(Br)c2n1] | Intermediate 9 and CAS- RN: [3197-42- 0] 10% LC-MS (Method 2): Rₜ = 1.27 min; MS (ESIpos): m/z = 441 [M + H]⁺ |
| | N-[(1H-benzimidazol-2-yl)methyl]-8-bromo-2-[(2S)-2- | |
| | methylpiperidin-1-yl]pyrazolo[1,5-a][1,3,5]triazin-4-amine | |
| | ¹H-NMR (400 MHz, DMSO-d6) δ [ppm]: −0.003 (9.14), 0.901 (2.71), | |
| | 0.966 (1.83), 1.107 (16.00), 1.144 (1.45), 1.195 (1.13), 1.231 (1.18), | |
| | 1.449 (4.88), 1.473 (3.73), 1.555 (2.45), 2.748 (1.38), 2.777 (2.50), | |
| | 2.810 (1.34), 4.197 (1.54), 4.439 (1.74), 4.471 (1.66), 4.836 (6.57), | |
| | 4.846 (7.04), 7.088 (1.00), 7.110 (4.66), 7.117 (5.55), 7.124 (5.11), | |
| | 7.133 (4.04), 7.147 (1.20), 7.379 (3.24), 7.396 (2.82), 7.519 (3.05), | |
| | 7.536 (2.93), 7.992 (14.90), 9.011 (2.92), 12.251 (4.46). | |
| 142 | 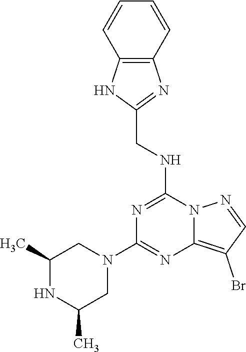 [SMILES: C[C@@H]1CN(c2nc(NCc3nc4ccccc4[nH]3)n3ncc(Br)c3n2)C[C@H](C)N1] | Intermediate 9 and CAS- RN: [21655- 48-1] 77% LC-MS (Method 2): Rₜ = 0.96 min; MS (ESIpos): m/z = 456 [M + H]⁺ |
| | N-(1H-benzimidazol-2-ylmethyl)-8-bromo-2-[(3R,5S)-3,5- | |
| | dimethylpiperazin-1-yl]pyrazolo[1,5-a][1,3,5]triazin-4-amine | |
| | ¹H-NMR (400 MHz, DMSO-d6) δ [ppm]: 0.798 (1.26), 0.919 (1.20), | |
| | 0.935 (1.32), 2.180 (0.72), 2.324 (1.12), 2.329 (1.42), 2.334 (1.16), | |
| | 2.338 (0.78), 2.520 (4.52), 2.525 (2.95), 2.542 (1.77), 2.557 (0.58), | |
| | 2.667 (0.78), 2.671 (1.08), 2.676 (0.80), 4.842 (4.51), 7.093 (0.60), | |
| | 7.107 (2.32), 7.111 (1.88), 7.117 (2.73), 7.123 (3.46), 7.130 (3.08), | |
| | 7.135 (2.03), 7.140 (2.62), 7.153 (0.71), 7.380 (1.96), 7.387 (1.25), | |
| | 7.398 (1.73), 7.402 (1.62), 7.516 (1.75), 7.520 (1.81), 7.538 (1.74), | |
| | 8.005 (16.00), 8.019 (0.56), 9.061 (0.58), 12.272 (2.72). | |
| 143 | 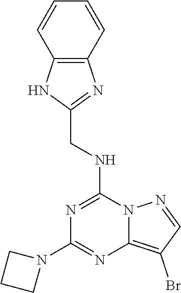 [SMILES: Brc1cnn2c(NCc3nc4ccccc4[nH]3)nc(N3CCC3)nc12] | Intermediate 9 and CAS- RN: [36520- 39-5] 36% LC-MS (Method 2): Rₜ = 1.04 min; MS (ESIpos): m/z = 399 [M + H]⁺ |
| | 2-(azetidin-1-yl)-N-[(1H-benzimidazol-2-yl)methyl]-8- | |
| | bromopyrazolo[1,5-a][1,3,5]triazin-4-amine | |
| | ¹H-NMR (400 MHz, DMSO-d6) δ [ppm]: 2.077 (4.93), 2.165 (0.90), | |
| | 2.183 (2.59), 2.203 (3.79), 2.221 (2.74), 2.240 (0.95), 2.334 (0.84), | |
| | 2.525 (2.80), 2.542 (3.51), 3.964 (4.40), 4.854 (7.30), 4.869 (7.26), | |
| | 7.127 (4.04), 7.134 (4.13), 7.141 (4.28), 7.149 (4.45), 7.481 (0.99), | |
| | 8.014 (16.00), 8.028 (0.48), 9.036 (1.64), 9.051 (3.57), 9.066 (1.64), | |
| | 12.231 (1.37). | |
| 144 | 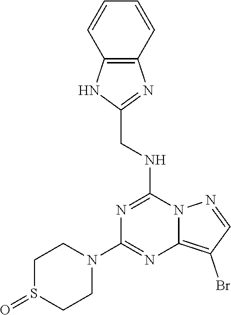 [SMILES: O=S1CCN(c2nc(NCc3nc4ccccc4[nH]3)n3ncc(Br)c3n2)CC1] | Intermediate 9 and CAS- RN: [76176- 87-9] 45% LC-MS (Method 2): Rₜ = 0.86 min; MS (ESIpos): m/z = 461 [M + H]⁺ |
| | 4-(4-{[(1H-benzimidazol-2-yl)methyl]amino}-8- | |
| | bromopyrazolo[1,5-a][1,3,5]triazin-2-yl)-1lambda⁴- | |
| | thiomorpholin-1-one | |
| | ¹H-NMR (400 MHz, DMSO-d6) δ [ppm]: 2.325 (0.88), 2.329 (1.18), | |
| | 2.334 (0.89), 2.525 (3.14), 2.542 (6.36), 2.667 (1.61), 2.671 (1.86), | |
| | 2.676 (1.56), 3.762 (1.58), 3.793 (2.63), 3.824 (1.68), 4.339 (1.86), | |
| | 4.372 (1.69), 4.867 (5.02), 4.882 (4.85), 7.107 (0.64), 7.120 (2.53), | |
| | 7.130 (3.62), 7.137 (3.44), 7.142 (3.88), 7.153 (2.68), 7.166 (0.68), | |
| | 7.403 (2.03), 7.420 (1.87), 7.535 (2.07), 7.553 (1.82), 8.070 (16.00), | |
| | 8.085 (0.69), 8.143 (0.99), 9.218 (1.67), 9.233 (3.52), 9.246 (1.68), | |
| | 12.250 (2.83). | |
| 145 | 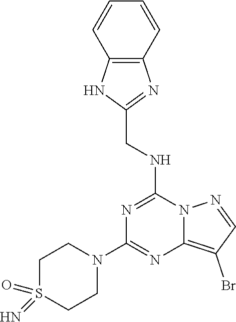 [SMILES: N=S1(=O)CCN(c2nc(NCc3nc4ccccc4[nH]3)n3ncc(Br)c3n2)CC1] | Intermediate 9 and CAS- RN: [1621962- 33-1] 22% LC-MS (Method 2): Rₜ = 0.82 min; MS (ESIpos): m/z = 476 [M + H]⁺ |
| | 4-(4-{[(1H-benzimidazol-2-yl)methyl]amino}-8- | |
| | bromopyrazolo[1,5-a][1,3,5]triazin-2-yl)-1-imino-1lambda⁶- | |
| | thiomorpholin-1-one | |
| | ¹H-NMR (400 MHz, DMSO-d6) δ [ppm]: 1.139 (0.46), 1.235 (1.36), | |
| | 1.320 (0.56), 1.427 (0.42), 1.908 (0.42), 2.077 (1.16), 2.325 (1.15), | |
| | 2.329 (1.59), 2.334 (1.19), 2.525 (4.59), 2.542 (14.93), 2.667 (1.47), | |
| | 2.671 (1.91), 2.676 (1.50), 2.807 (0.71), 2.997 (0.54), 3.756 (6.85), | |
| | 4.266 (1.42), 4.874 (6.59), 7.109 (0.81), 7.123 (2.81), 7.128 (2.45), | |
| | 7.134 (3.32), 7.140 (4.34), 7.146 (3.65), 7.152 (2.59), 7.156 (3.06), | |
| | 7.170 (0.87), 7.391 (2.56), 7.407 (2.25), 7.539 (2.44), 7.556 (2.25), | |
| | 8.091 (16.00), 9.287 (1.43), 12.211 (3.06). | |
| 146 | 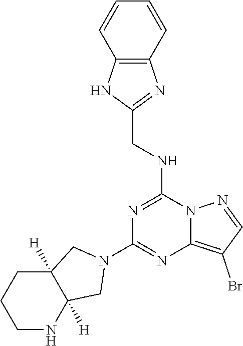 [SMILES: Brc1cnn2c(NCc3nc4ccccc4[nH]3)nc(N3C[C@@H]4CCCN[C@@H]4C3)nc12] | Intermediate 9 and CAS- RN: [151213- 40-0] 56% LC-MS (Method 2): Rₜ = 1.06 min; MS (ESIpos): m/z = 468 [M + H]⁺ |
| | N-[(1H-benzimidazol-2-yl)methyl]-8-bromo-2-[(4aS,7aS)- | |
| | octahydro-6H-pyrrolo[3,4-b]pyridin-6-yl]pyrazolo[1,5- | |
| | a][1,3,5]triazin-4-amine | |
| | ¹H-NMR (400 MHz, DMSO-d6) δ [ppm]: 1.303 (1.23), 1.346 (1.51), | |
| | 1.485 (0.80), 1.497 (0.80), 1.520 (1.12), 1.534 (1.31), 1.546 (1.66), | |
| | 1.570 (2.01), 1.663 (1.04), 1.696 (0.54), 2.143 (1.04), 2.154 (1.03), | |
| | 2.166 (0.98), 2.398 (1.22), 2.425 (2.28), 2.452 (1.32), 2.525 (3.95), | |
| | 2.542 (16.00), 2.735 (2.10), 2.765 (1.82), 3.144 (1.65), 3.181 (2.11), | |
| | 3.283 (1.25), 3.309 (3.93), 3.367 (4.03), 3.387 (3.94), 3.395 (2.94), | |
| | 3.415 (1.40), 3.434 (2.95), 3.447 (2.19), 3.464 (1.60), 3.473 (1.81), | |
| | 3.481 (1.73), 3.502 (1.76), 3.529 (0.71), 4.829 (0.45), 4.883 (6.66), | |
| | 4.922 (0.48), 7.101 (0.74), 7.114 (3.13), 7.123 (4.40), 7.130 (4.27), | |
| | 7.137 (4.91), 7.146 (3.46), 7.159 (0.90), 7.400 (2.45), 7.417 (2.29), | |
| | 7.529 (2.52), 7.548 (2.34), 7.973 (9.64), 7.981 (8.35), 8.958 (1.11), | |
| | 12.246 (1.61), 12.269 (1.90). | |
| 147 | 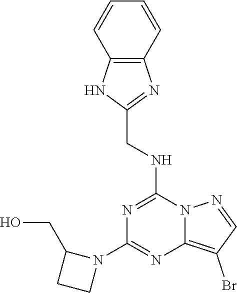 [SMILES: OCC1CCN1c1nc(NCc2nc3ccccc3[nH]2)n2ncc(Br)c2n1] | Intermediate 9 and CAS- RN: [250274- 91-0] 58% LC-MS (Method 2): Rₜ = 0.94 min; MS (ESIpos): m/z = 429 [M + H]⁺ |
| | [1-[4-(1H-benzimidazol-2-ylmethylamino)-8-bromo-pyrazolo[1,5- | |
| | a][1,3,5]triazin-2-yl]azetidin-2-yl]methanol | |
| | ¹H-NMR (400 MHz, DMSO-d6) δ [ppm]: 2.077 (3.00), 2.087 (1.75), | |
| | 2.114 (1.56), 2.165 (1.37), 2.185 (2.65), 2.205 (2.97), 2.223 (1.67), | |
| | 2.249 (0.63), 2.334 (1.92), 2.521 (10.68), 2.525 (6.74), 2.542 (4.92), | |
| | 2.676 (1.94), 3.514 (0.82), 3.707 (2.80), 3.845 (2.57), 4.249 (0.80), | |
| | 4.388 (0.73), 4.717 (0.67), 4.860 (12.90), 5.170 (0.62), 7.132 (7.24), | |
| | 7.144 (7.83), 7.409 (3.19), 7.425 (3.02), 7.534 (3.20), 7.552 (2.80), | |
| | 8.020 (16.00), 9.075 (1.14), 12.219 (2.46). | |
| 148 | 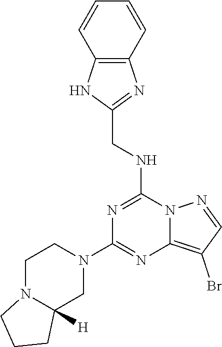 [SMILES: Brc1cnn2c(NCc3nc4ccccc4[nH]3)nc(N3CCN4CCC[C@H]4C3)nc12] | Intermediate 9 and CAS- RN: [93643- 24-4] 60% LC-MS (Method 2): Rₜ = 1.10 min; MS (ESIpos): m/z = 468 [M + H]⁺ Specific Optical Rotation (Method O1): −18.3° (c = 10 mg/mL, DMSO) |
| | N-[(1H-benzimidazol-2-yl)methyl]-8-bromo-2-[(8aS)- | |
| | hexahydropyrrolo[1, 2-a]pyrazin-2 (1 H)-yl]pyrazolo[1,5- | |
| | a][1,3,5]triazin-4-amine | |
| | ¹H-NMR (400 MHz, DMSO-d6) δ [ppm]: 1.235 (0.62), 1.643 (1.55), | |
| | 1.896 (1.33), 1.917 (2.39), 1.938 (2.10), 2.809 (1.43), 2.836 (1.10), | |
| | 2.910 (2.09), 2.931 (3.28), 2.951 (1.68), 4.529 (1.07), 4.855 (5.25), | |
| | 4.869 (5.14), 7.100 (1.05), 7.115 (3.37), 7.118 (3.26), 7.123 (3.96), | |
| | 7.130 (5.68), 7.137 (4.35), 7.142 (3.46), 7.146 (3.69), 7.161 (1.15), | |
| | 7.387 (3.21), 7.404 (2.73), 7.528 (2.92), 7.545 (2.85), 8.021 (16.00), | |
| | 9.074 (1.72), 9.089 (3.38), 9.102 (1.68), 12.263 (4.32). | |
| 149 | 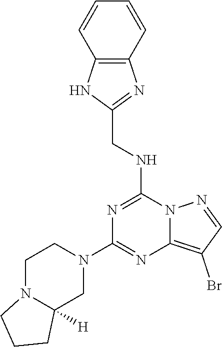 [SMILES: Brc1cnn2c(NCc3nc4ccccc4[nH]3)nc(N3CCN4CCC[C@@H]4C3)nc12] | Intermediate 9 and CAS- RN: [96193- 27-0] 62% LC-MS (Method 2): Rₜ = 1.10 min; MS (ESIpos): m/z = 468 [M + H]⁺ Specific Optical Rotation (Method O1): +22.9° (c = 10 mg/mL, DMSO) |
| | N-[(1H-benzimidazol-2-yl)methyl]-8-bromo-2-[(8aR)- | |
| | hexahydropyrrolo[1, 2-a]pyrazin-2 (1 H)-yl]pyrazolo[1,5- | |
| | a][1,3,5]triazin-4-amine | |
| | ¹H-NMR (400 MHz, DMSO-d6) δ [ppm]: 1.641 (0.89), 1.895 (0.79), | |
| | 1.917 (1.48), 1.938 (1.30), 1.959 (0.45), 2.520 (4.11), 2.525 (2.67), | |
| | 2.808 (0.84), 2.839 (0.63), 2.910 (1.27), 2.931 (2.05), 2.951 (1.02), | |
| | 4.535 (0.60), 4.855 (3.26), 4.869 (3.17), 7.099 (0.70), 7.113 (2.44), | |
| | 7.118 (2.08), 7.123 (2.76), 7.130 (4.05), 7.136 (3.04), 7.142 (2.18), | |
| | 7.146 (2.70), 7.160 (0.78), 7.387 (2.08), 7.393 (1.27), 7.403 (1.84), | |
| | 7.408 (1.66), 7.523 (1.88), 7.527 (1.93), 7.545 (1.85), 8.021 (16.00), | |
| | 9.074 (1.03), 9.089 (2.11), 9.103 (1.02), 12.262 (2.77). | |
| 150 | 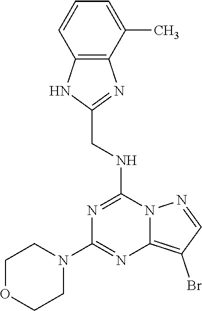 [SMILES: Cc1cccc2[nH]c(CNc3nc(N4CCOCC4)nc4c(Br)cnn34)nc12] | Intermediate 111 and CAS- RN: [110-91-8] 19% LC-MS (Method 2): Rₜ = 1.11 min; MS (ESIpos): m/z = 443 [M + H]⁺ |
| | 8-bromo-N-[(4-methyl-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: 2.457 (9.53), 2.520 (3.94), | |
| | 2.525 (2.67), 2.542 (2.30), 3.502 (3.87), 3.632 (6.15), 4.860 (2.35), | |
| | 4.871 (4.65), 4.885 (3.04), 6.918 (1.67), 6.936 (3.00), 6.952 (1.71), | |
| | 6.999 (1.59), 7.007 (2.00), 7.019 (2.08), 7.027 (2.81), 7.037 (1.16), | |
| | 7.045 (1.64), 7.199 (2.23), 7.219 (1.80), 7.329 (1.46), 7.349 (1.27), | |
| | 8.036 (16.00), 9.053 (0.45), 9.067 (0.87), 9.082 (0.44), 9.138 (0.65), | |
| | 9.152 (1.26), 9.166 (0.60), 12.178 (2.08), 12.272 (1.41). | |
| 151 | 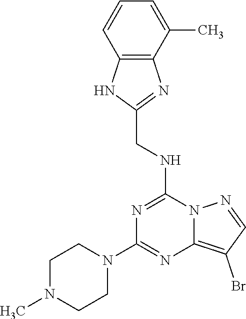 [SMILES: Cc1cccc2[nH]c(CNc3nc(N4CCN(C)CC4)nc4c(Br)cnn34)nc12] | Intermediate 111 and CAS- RN: [109-01-3] 40% LC-MS (Method 2): Rₜ = 1.08 min; MS (ESIneg): m/z = 454 [M − H]⁻ |
| | 8-bromo-N-[(4-methyl-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: 2.122 (12.25), 2.128 | |
| | (15.40), 2.194 (2.84), 2.459 (10.38), 2.520 (5.55), 2.525 (3.69), | |
| | 2.542 (1.00), 3.638 (5.26), 4.852 (2.55), 4.863 (5.04), 4.876 (3.27), | |
| | 6.917 (1.78), 6.935 (3.10), 6.949 (1.85), 6.998 (1.69), 7.006 (2.11), | |
| | 7.018 (2.29), 7.025 (2.96), 7.036 (1.24), 7.044 (1.70), 7.196 (2.35), | |
| | 7.215 (1.91), 7.328 (1.60), 7.347 (1.38), 8.017 (16.00), 9.018 (0.94), | |
| | 9.086 (0.72), 9.100 (1.37), 9.114 (0.66), 12.176 (2.32), 12.271 | |
| | (1.57). | |
| 152 | 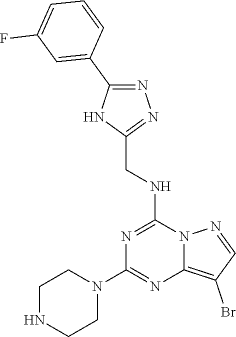 [SMILES: Fc1cccc(-c2nnc(CNc3nc(N4CCNCC4)nc4c(Br)cnn34)[nH]2)c1] | Intermediate 109 and CAS- RN: [110-85-0] 50% LC-MS (Method 2): Rₜ = 0.73 min; MS (ESIpos): m/z = 473 [M + H]⁺ |
| | 8-bromo-N-{[5-(3-fluorophenyl)-4H-1,2,4-triazol-3-yl]methyl}-2- | |
| | (piperazin-1-yl)pyrazolo[1,5-a][1,3,5]triazin-4-amine | |
| | ¹H-NMR (400 MHz, DMSO-d6) δ [ppm]: 2.325 (0.73), 2.330 (1.01), | |
| | 2.335 (0.72), 2.521 (3.87), 2.526 (2.59), 2.543 (0.89), 2.605 (3.90), | |
| | 2.667 (0.93), 2.672 (1.16), 2.677 (0.86), 3.589 (5.59), 4.768 (9.45), | |
| | 7.240 (0.96), 7.245 (1.04), 7.247 (1.02), 7.261 (1.92), 7.267 (2.05), | |
| | 7.283 (1.10), 7.288 (1.15), 7.491 (1.38), 7.507 (1.63), 7.512 (2.46), | |
| | 7.527 (2.42), 7.532 (1.57), 7.547 (1.34), 7.676 (1.63), 7.680 (1.88), | |
| | 7.683 (1.95), 7.686 (1.76), 7.702 (1.64), 7.705 (1.94), 7.708 (1.85), | |
| | 7.712 (1.67), 7.803 (2.50), 7.806 (3.48), 7.826 (3.00), 7.995 (16.00). | |
| 153 | 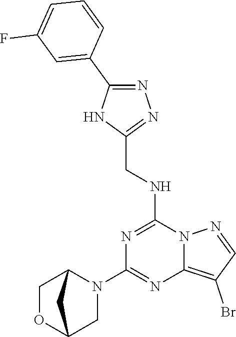 [SMILES: Fc1cccc(-c2nnc(CNc3nc(N4C[C@H]5C[C@@H]4CO5)nc4c(Br)cnn34)[nH]2)c1] | Intermediate 109 and CAS- RN: [601515- 79-1] 44% LC-MS (Method 2): Rₜ = 0.77 min; MS (ESIneg): m/z = 484 [M − H]⁻ Specific Optical Rotation (Method O1): +54.3° (c = 10 mg/mL, DMSO) |
| | 8-bromo-N-{[5-(3-fluorophenyl)-4H-1,2,4-triazol-3-yl]methyl}-2- | |
| | [(1R,4R)-2-oxa-5-azabicyclo[2.2.1]heptan-5-yl]pyrazolo[1,5- | |
| | a][1,3,5]triazin-4-amine | |
| | ¹H-NMR (400 MHz, DMSO-d6) δ [ppm]: 1.739 (0.49), 1.762 (0.91), | |
| | 1.816 (1.56), 1.839 (1.23), 2.335 (0.81), 2.521 (4.05), 2.526 (2.63), | |
| | 2.543 (2.32), 2.677 (0.82), 3.401 (2.25), 3.421 (1.32), 3.551 (0.90), | |
| | 3.567 (0.75), 3.673 (0.43), 3.691 (0.51), 3.768 (0.49), 4.588 (3.42), | |
| | 4.783 (4.23), 4.821 (1.44), 4.881 (0.75), 7.237 (0.71), 7.243 (0.77), | |
| | 7.260 (1.44), 7.265 (1.51), 7.281 (0.84), 7.286 (0.86), 7.488 (0.91), | |
| | 7.508 (1.78), 7.524 (1.76), 7.544 (0.90), 7.680 (1.55), 7.705 (1.53), | |
| | 7.805 (2.63), 7.824 (2.30), 8.010 (16.00), 9.119 (0.55). | |
| 154 | 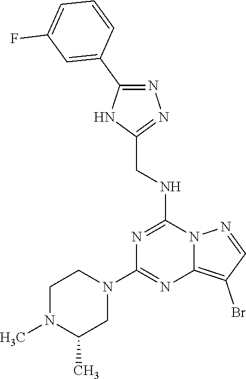 [SMILES: C[C@H]1CN(c2nc(NCc3nnc(-c4cccc(F)c4)[nH]3)n3ncc(Br)c3n2)CCN1C] | Intermediate 109 and CAS- RN: [485841- 50-7] 37% LC-MS (Method 2): Rₜ = 0.95 min; MS (ESIpos): m/z = 501 [M + H]⁺ Specific Optical Rotation (Method O1): −14.6° (c = 10 mg/mL, DMSO) |
| | 8-bromo-2-[(3S)-3,4-dimethylpiperazin-1-yl]-N-{[5-(3- | |
| | fluorophenyl)-4H-1,2,4-triazol-3-yl]methyl}pyrazolo[1,5- | |
| | a][1,3,5]triazin-4-amine | |
| | ¹H-NMR (400 MHz, DMSO-d6) δ [ppm]: 0.916 (1.01), 1.852 (0.41), | |
| | 1.952 (0.65), 2.120 (16.00), 2.325 (0.83), 2.330 (1.14), 2.335 (0.81), | |
| | 2.521 (3.94), 2.526 (2.53), 2.543 (0.65), 2.565 (0.80), 2.596 (0.98), | |
| | 2.622 (0.79), 2.663 (0.88), 2.667 (1.33), 2.672 (1.61), 2.677 (1.31), | |
| | 2.681 (0.87), 2.981 (0.55), 4.327 (1.06), 4.358 (0.98), 4.767 (2.83), | |
| | 4.774 (2.84), 7.245 (0.75), 7.261 (1.28), 7.267 (1.34), 7.282 (0.74), | |
| | 7.288 (0.72), 7.490 (0.93), 7.506 (1.18), 7.510 (1.64), 7.526 (1.58), | |
| | 7.530 (1.05), 7.546 (0.78), 7.676 (1.19), 7.679 (1.41), 7.682 (1.44), | |
| | 7.685 (1.30), 7.701 (1.22), 7.704 (1.44), 7.707 (1.40), 7.711 (1.27), | |
| | 7.802 (2.01), 7.805 (2.76), 7.808 (1.96), 7.822 (1.79), 7.825 (2.37), | |
| | 7.828 (1.66), 8.008 (15.69), 8.274 (0.52), 9.106 (0.98). | |
| 155 | 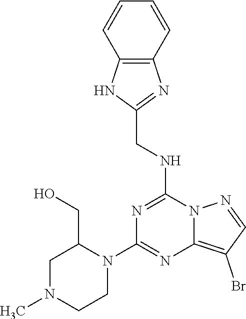 [SMILES: CN1CCN(c2nc(NCc3nc4ccccc4[nH]3)n3ncc(Br)c3n2)C(CO)C1] | Intermediate 9 and CAS- RN: [85155- 83-5] 34% LC-MS (Method 2): Rt = 0.98 min; MS (ESIneg): m/z = 470 [M − H]⁻ |
| | [1-[4-(1H-benzimidazol-2-ylmethylamino)-8-bromo-pyrazolo[1,5- | |
| | a][1,3,5]triazin-2-yl]-4-methyl-piperazin-2-yl]methanol | |
| | ¹H-NMR (400 MHz, DMSO-d6) δ [ppm]: 1.816 (1.44), 1.837 (1.42), | |
| | 2.077 (0.43), 2.123 (7.34), 2.335 (1.13), 2.339 (0.54), 2.521 (5.23), | |
| | 2.526 (3.51), 2.543 (3.64), 2.677 (1.24), 2.681 (0.62), 2.909 (0.88), | |
| | 3.303 (0.61), 3.386 (0.93), 3.643 (0.71), 4.432 (0.47), 4.675 (1.65), | |
| | 4.683 (1.64), 4.861 (2.02), 4.873 (2.06), 7.108 (0.99), 7.122 (3.18), | |
| | 7.126 (2.73), 7.133 (3.44), 7.139 (5.06), 7.145 (3.87), 7.152 (2.80), | |
| | 7.156 (3.29), 7.170 (1.05), 7.412 (2.23), 7.430 (1.93), 7.533 (2.53), | |
| | 7.537 (2.63), 7.553 (2.47), 8.008 (16.00), 9.059 (1.14), 9.073 (2.04), | |
| | 12.180 (0.56). | |
| 156 | 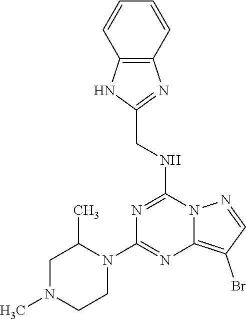 [SMILES: CC1CN(C)CCN1c1nc(NCc2nc3ccccc3[nH]2)n2ncc(Br)c2n1] | Intermediate 9 and CAS- RN: [22317- 01-7] 18% LC-MS (Method 2): Rₜ = 1.10 min; MS (ESIpos): m/z = 456 [M + H]⁺ |
| | N-(1H-benzimidazol-2-ylmethyl)-8-bromo-2-(2,4- | |
| | dimethylpiperazin-1-yl)pyrazolo[1,5-a][1,3,5]triazin-4-amine | |
| | ¹H-NMR (400 MHz, DMSO-d6) δ [ppm]: −0.008 (1.06), 0.008 (0.90), | |
| | 0.955 (0.72), 1.706 (0.53), 1.889 (0.83), 1.898 (0.90), 1.917 (0.95), | |
| | 1.926 (0.87), 2.110 (13.95), 2.153 (1.20), 2.180 (0.63), 2.325 (0.72), | |
| | 2.329 (0.96), 2.334 (0.71), 2.521 (3.13), 2.525 (2.15), 2.542 (16.00), | |
| | 2.569 (0.69), 2.667 (0.93), 2.672 (1.22), 2.676 (1.03), 2.681 (0.77), | |
| | 2.695 (0.67), 2.708 (0.69), 2.931 (0.67), 2.939 (0.75), 2.963 (1.19), | |
| | 2.970 (1.11), 2.994 (0.79), 3.002 (0.66), 3.502 (0.82), 4.286 (1.11), | |
| | 4.318 (1.04), 4.853 (2.70), 4.864 (1.97), 7.103 (0.56), 7.113 (2.87), | |
| | 7.120 (3.02), 7.128 (3.10), 7.135 (3.40), 7.144 (0.87), 7.416 (0.61), | |
| | 7.453 (0.71), 7.473 (0.82), 7.491 (0.67), 7.505 (0.56), 7.509 (0.56), | |
| | 7.937 (0.43), 8.018 (11.38), 8.048 (0.77), 8.157 (7.27), 9.083 (1.11). | |
| 157 | 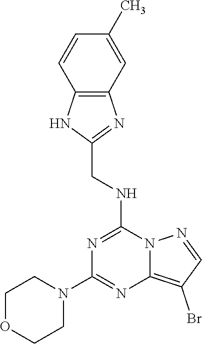 [SMILES: Cc1ccc2[nH]c(CNc3nc(N4CCOCC4)nc4c(Br)cnn34)nc2c1] | Intermediate 113 and CAS- RN: [110-91-8] 20% LC-MS (Method 2): Rₜ = 1.09 min; MS (ESIpos): m/z = 443 [M + H]⁺ |
| | 8-bromo-N-[(5-methyl-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: 0.000 (4.72), 2.079 (0.20), | |
| | 2.086 (16.00), 2.381 (3.09), 2.521 (1.40), 2.525 (0.89), 2.542 (0.48), | |
| | 3.507 (1.01), 3.630 (1.46), 4.846 (1.54), 6.934 (0.21), 6.954 (0.36), | |
| | 7.191 (0.32), 7.324 (0.24), 7.391 (0.21), 7.410 (0.19), 8.035 (4.28), | |
| | 9.108 (0.20), 12.105 (0.21). | |
| 158 | 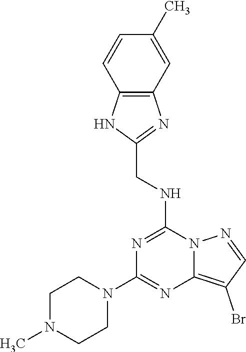 [SMILES: Cc1ccc2[nH]c(CNc3nc(N4CCN(C)CC4)nc4c(Br)cnn34)nc2c1] | Intermediate 113 and CAS- RN: [109-01-3] 25% LC-MS (Method 2): Rₜ = 1.06 min; MS (ESIpos): m/z = 456 [M + H]⁺ |
| | 8-bromo-N-[(5-methyl-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: −0.008 (1.28), 0.008 (1.44), | |
| | 2.126 (16.00), 2.143 (1.20), 2.197 (1.92), 2.334 (2.40), 2.338 (1.12), | |
| | 2.376 (6.56), 2.384 (8.16), 2.520 (12.16), 2.524 (7.92), 2.542 (0.96), | |
| | 2.566 (0.88), 2.676 (2.40), 2.680 (1.04), 3.632 (3.20), 4.830 (2.80), | |
| | 4.843 (2.88), 6.929 (0.96), 6.951 (1.68), 6.973 (0.80), 7.187 (1.84), | |
| | 7.263 (1.28), 7.284 (1.12), 7.323 (1.44), 7.389 (1.52), 7.410 (1.36), | |
| | 8.014 (12.80), 9.051 (1.12), 12.093 (1.28), 12.118 (1.04). | |
| 159 | 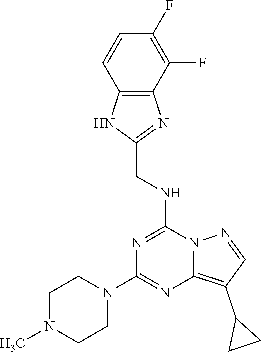 [SMILES: CN1CCN(c2nc(NCc3nc4c(F)c(F)ccc4[nH]3)n3ncc(C4CC4)c3n2)CC1] | Intermediate 115 and CAS- RN: [109-01-3] 45% LC-MS (Method 2): Rₜ = 1.07 min; MS (ESIpos): m/z = 440 [M + H]⁺ |
| | 8-cyclopropyl-N-[(4,5-difluoro-1H-benzimidazol-2-yl)methyl]-2- | |
| | (4-methylpiperazin-1-yl)pyrazolo[1,5-a][1,3,5]triazin-4-amine | |
| | ¹H-NMR (400 MHz, DMSO-d6) δ [ppm]: 0.700 (0.91), 0.705 (0.66), | |
| | 0.713 (2.93), 0.718 (3.77), 0.725 (4.25), 0.731 (3.87), 0.738 (1.80), | |
| | 0.751 (0.59), 0.757 (0.73), 0.764 (1.83), 0.772 (3.66), 0.777 (2.37), | |
| | 0.785 (2.47), 0.789 (1.53), 0.793 (3.66), 0.798 (2.34), 0.804 (0.77), | |
| | 0.810 (1.05), 1.701 (0.59), 1.714 (1.11), 1.721 (1.13), 1.734 (1.81), | |
| | 1.742 (0.68), 1.747 (1.06), 1.755 (1.01), 1.768 (0.48), 2.106 (16.00), | |
| | 2.151 (3.27), 2.334 (1.60), 2.338 (0.72), 2.520 (8.48), 2.524 (5.44), | |
| | 2.542 (0.74), 2.676 (1.61), 2.680 (0.74), 3.565 (4.01), 4.828 (3.04), | |
| | 7.146 (0.48), 7.168 (1.10), 7.174 (0.64), 7.186 (1.14), 7.196 (1.29), | |
| | 7.213 (1.42), 7.695 (9.93), 8.867 (0.74). | |
| 160 | 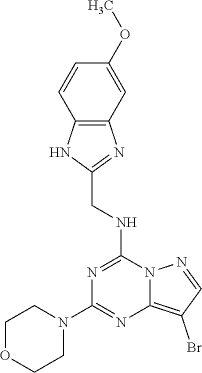 [SMILES: COc1ccc2[nH]c(CNc3nc(N4CCOCC4)nc4c(Br)cnn34)nc2c1] | Intermediate 117 and CAS- RN: [110-91-8] 20% LC-MS (Method 2): Rₜ = 1.01 min; MS (ESIneg): m/z = 457 [M − H]⁻ |
| | 8-bromo-N-[(5-methoxy-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: 2.329 (1.51), 2.334 (1.09), | |
| | 2.520 (6.74), 2.524 (4.25), 2.542 (8.16), 2.671 (1.52), 2.676 (1.12), | |
| | 3.514 (2.86), 3.636 (3.87), 3.749 (16.00), 4.831 (3.65), 6.742 (0.60), | |
| | 6.760 (0.95), 6.896 (0.88), 7.070 (0.56), 7.398 (0.62), 7.420 (0.56), | |
| | 8.033 (10.36), 9.098 (0.60), 12.056 (0.51). | |
| 161 | 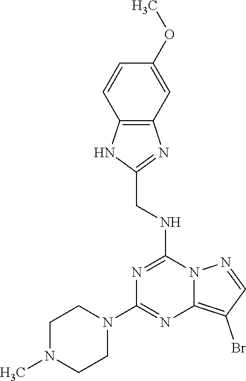 [SMILES: COc1ccc2[nH]c(CNc3nc(N4CCN(C)CC4)nc4c(Br)cnn34)nc2c1] | Intermediate 117 and CAS- RN: [109-01-3] 32% LC-MS (Method 2): Rₜ = 0.99 min; MS (ESIpos): m/z = 472 [M + H]⁺ |
| | 8-bromo-N-[(5-methoxy-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: 0.000 (3.54), 2.086 (8.28), | |
| | 2.129 (9.84), 2.203 (1.67), 2.325 (0.46), 2.329 (0.59), 2.334 (0.44), | |
| | 2.525 (1.78), 2.542 (1.36), 2.667 (0.38), 2.672 (0.53), 2.676 (0.40), | |
| | 3.567 (0.16), 3.641 (2.58), 3.749 (16.00), 4.824 (3.52), 6.747 (0.64), | |
| | 6.768 (0.71), 6.896 (0.42), 7.070 (0.26), 7.285 (0.20), 7.397 (0.31), | |
| | 8.014 (7.11), 9.048 (0.38), 12.071 (0.31). | |
| 162 | 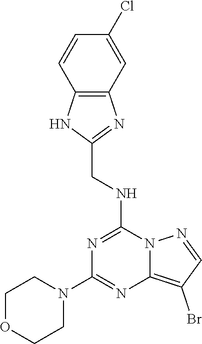 [SMILES: Clc1ccc2[nH]c(CNc3nc(N4CCOCC4)nc4c(Br)cnn34)nc2c1] | Intermediate 119 and CAS- RN: [110-91-8] 17% LC-MS (Method 2): Rₜ = 1.13 min; MS (ESIpos): m/z = 463 [M + H]⁺ |
| | 8-bromo-N-[(5-chloro-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: −0.008 (2.56), 0.008 (2.24), | |
| | 2.335 (1.12), 2.521 (5.60), 2.526 (3.52), 2.543 (0.64), 2.677 (1.12), | |
| | 3.427 (0.48), 3.490 (3.52), 3.610 (4.96), 4.862 (4.00), 4.874 (3.84), | |
| | 7.165 (1.28), 7.182 (0.96), 7.415 (0.80), 7.435 (0.80), 7.460 (0.96), | |
| | 7.539 (0.64), 7.558 (0.48), 7.600 (1.12), 8.041 (16.00), 9.162 (1.60), | |
| | 12.433 (0.64), 12.478 (0.80). | |
| 163 | 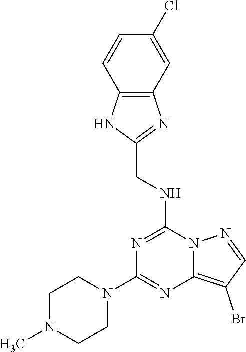 [SMILES: CN1CCN(c2nc(NCc3nc4cc(Cl)ccc4[nH]3)n3ncc(Br)c3n2)CC1] | Intermediate 119 and CAS- RN: [109-01-3] 26% LC-MS (Method 2): Rₜ = 1.11 min; MS (ESIpos): m/z = 476 [M + H]⁺ |
| | 8-bromo-N-[(5-chloro-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: −0.008 (1.96), 0.008 (1.67), | |
| | 2.117 (16.00), 2.164 (1.40), 2.320 (0.47), 2.325 (0.98), 2.329 (1.31), | |
| | 2.334 (0.96), 2.339 (0.45), 2.521 (4.21), 2.526 (2.76), 2.542 (0.96), | |
| | 2.662 (0.42), 2.667 (0.94), 2.672 (1.27), 2.676 (0.94), 2.681 (0.40), | |
| | 3.610 (2.41), 4.851 (2.74), 4.863 (2.70), 7.162 (1.05), 7.179 (0.71), | |
| | 7.409 (0.74), 7.430 (0.69), 7.453 (0.80), 7.536 (0.56), 7.556 (0.49), | |
| | 7.598 (1.00), 8.023 (13.64), 9.114 (1.14), 12.431 (0.56), 12.476 | |
| | (0.71). | |
| 164 | 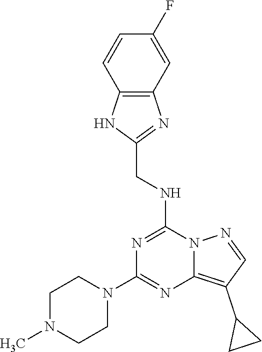 [SMILES: CN1CCN(c2nc(NCc3nc4cc(F)ccc4[nH]3)n3ncc(C4CC4)c3n2)CC1] | Intermediate 121 and CAS- RN: [109-01-3] 18% LC-MS (Method 1): Rₜ = 0.69 min; MS (ESIpos): m/z = 422 [M + H]⁺ |
| | 8-cyclopropyl-N-[(5-fluoro-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: −0.008 (3.71), 0.008 (3.56), | |
| | 0.146 (0.43), 0.701 (1.66), 0.706 (1.24), 0.714 (5.22), 0.719 (6.61), | |
| | 0.726 (7.38), 0.732 (6.61), 0.739 (3.21), 0.753 (1.04), 0.759 (1.31), | |
| | 0.766 (3.21), 0.774 (6.69), 0.780 (4.17), 0.787 (4.37), 0.792 (2.78), | |
| | 0.795 (6.38), 0.801 (4.17), 0.806 (1.47), 0.812 (1.86), 1.068 (8.23), | |
| | 1.225 (2.40), 1.234 (1.55), 1.336 (1.31), 1.351 (1.00), 1.703 (1.00), | |
| | 1.716 (1.93), 1.724 (1.97), 1.737 (3.25), 1.745 (1.24), 1.750 (1.93), | |
| | 1.754 (1.58), 1.758 (1.86), 1.771 (0.85), 2.131 (13.80), 2.194 (5.14), | |
| | 2.290 (0.58), 2.456 (0.62), 2.460 (0.93), 2.465 (1.16), 2.469 (1.24), | |
| | 2.521 (8.15), 2.525 (5.37), 2.533 (1.00), 2.538 (0.81), 2.542 (1.31), | |
| | 2.662 (0.73), 3.040 (0.50), 3.583 (6.11), 4.819 (4.56), 4.832 (3.71), | |
| | 5.761 (3.17), 6.942 (0.62), 6.948 (0.73), 6.971 (1.78), 6.995 (1.93), | |
| | 7.018 (0.85), 7.023 (0.85), 7.186 (1.24), 7.191 (1.24), 7.208 (1.24), | |
| | 7.214 (1.20), 7.311 (1.35), 7.316 (1.31), 7.335 (1.35), 7.341 (1.31), | |
| | 7.372 (1.35), 7.384 (1.43), 7.393 (1.35), 7.405 (1.24), 7.505 (1.08), | |
| | 7.518 (1.20), 7.527 (1.16), 7.539 (1.08), 7.632 (0.46), 7.692 (16.00), | |
| | 8.816 (2.01), 8.830 (2.09), 12.343 (1.82), 12.363 (2.01). | |
| 165 | 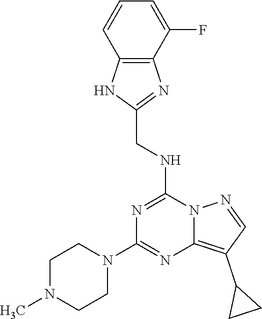 [SMILES: CN1CCN(c2nc(NCc3nc4c(F)cccc4[nH]3)n3ncc(C4CC4)c3n2)CC1] | Intermediate 123 and CAS- RN: [109-01-3] 14% LC-MS (Method 1): Rₜ = 0.75 min; MS (ESIpos): m/z = 422 [M + H]⁺ |
| | 8-cyclopropyl-N-[(4-fluoro-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: −0.008 (3.34), 0.008 (3.15), | |
| | 0.690 (0.50), 0.698 (0.61), 0.706 (2.07), 0.711 (1.65), 0.718 (6.71), | |
| | 0.723 (8.25), 0.731 (9.29), 0.737 (8.79), 0.744 (3.76), 0.756 (1.50), | |
| | 0.763 (1.80), 0.769 (4.76), 0.777 (8.79), 0.782 (6.10), 0.790 (6.29), | |
| | 0.794 (3.95), 0.797 (9.13), 0.802 (5.79), 0.809 (1.84), 0.815 (2.53), | |
| | 1.068 (0.84), 1.235 (1.50), 1.707 (1.38), 1.720 (2.61), 1.728 (2.72), | |
| | 1.740 (4.37), 1.749 (1.69), 1.754 (2.76), 1.761 (2.42), 1.774 (1.11), | |
| | 2.192 (7.83), 2.235 (2.57), 2.282 (4.03), 2.292 (4.11), 2.320 (2.57), | |
| | 2.325 (3.26), 2.329 (3.68), 2.334 (2.84), 2.521 (7.75), 2.525 (5.14), | |
| | 2.542 (1.53), 2.662 (0.77), 2.667 (1.65), 2.671 (2.26), 2.676 (1.61), | |
| | 3.607 (4.64), 4.840 (10.13), 4.855 (10.13), 5.761 (1.19), 6.910 | |
| | (2.30), 6.929 (2.99), 6.938 (2.53), 6.956 (2.88), 7.000 (0.58), 7.026 | |
| | (0.50), 7.093 (1.96), 7.105 (2.15), 7.113 (3.95), 7.125 (3.76), 7.133 | |
| | (2.30), 7.145 (2.00), 7.232 (5.95), 7.251 (4.34), 7.368 (0.81), 7.388 | |
| | (0.73), 7.643 (1.04), 7.706 (16.00), 8.827 (0.58), 8.878 (1.53), 8.892 | |
| | (2.99), 8.907 (1.50), 12.561 (3.45), 12.907 (0.77). | |
| 166 | 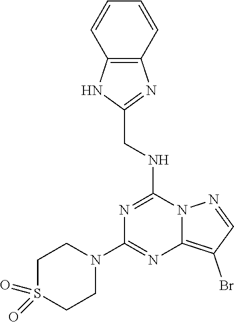 [SMILES: O=S1(=O)CCN(c2nc(NCc3nc4ccccc4[nH]3)n3ncc(Br)c3n2)CC1] | Intermediate 9 and CAS- RN: [39093- 93-1] 7% LC-MS (Method 1): Rₜ = 0.76 min; MS (ESIpos): m/z = 477 [M + H]⁺ |
| | 4-(4-{[(1H-benzimidazol-2-yl)methyl]amino}-8- | |
| | bromopyrazolo[1,5-a][1,3,5]triazin-2-yl)-1 lambda⁶- | |
| | thiomorpholine-1,1-dione | |
| | ¹H-NMR (400 MHz, DMSO-d6) δ [ppm]: −0.008 (1.34), 0.008 (1.34), | |
| | 2.076 (0.62), 2.324 (0.99), 2.329 (1.34), 2.334 (0.96), 2.525 (3.68), | |
| | 2.542 (5.32), 2.666 (0.99), 2.671 (1.31), 2.676 (0.99), 2.994 (1.04), | |
| | 4.094 (5.34), 4.875 (4.03), 4.889 (3.98), 7.137 (3.07), 7.147 (3.44), | |
| | 7.401 (1.51), 7.418 (1.43), 7.541 (1.78), 7.559 (1.53), 8.104 (16.00), | |
| | 8.148 (0.67), 9.300 (1.21), 9.314 (2.25), 12.205 (1.88). | |
| 167 | 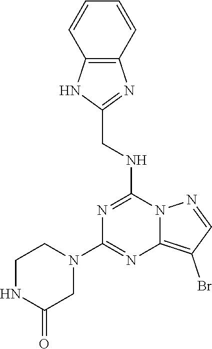 [SMILES: O=C1CN(c2nc(NCc3nc4ccccc4[nH]3)n3ncc(Br)c3n2)CCN1] | Intermediate 9 and CAS- RN: [5625-67- 2] 35% LC-MS (Method 2): Rₜ = 0.84 min; MS (ESIpos): m/z = 442 [M + H]⁺ |
| | 4-(4-{[(1H-benzimidazol-2-yl)methyl]amino}-8- | |
| | bromopyrazolo[1,5-a][1,3,5]triazin-2-yl)piperazin-2-one | |
| | ¹H-NMR (400 MHz, DMSO-d6) δ [ppm]: −0.008 (1.40), 0.008 (1.32), | |
| | 1.235 (0.45), 2.086 (1.77), 2.334 (0.76), 2.520 (3.83), 2.525 (2.46), | |
| | 2.542 (3.93), 2.676 (0.78), 3.806 (1.54), 4.144 (3.42), 4.898 (4.11), | |
| | 7.102 (0.61), 7.116 (2.29), 7.120 (1.90), 7.126 (2.68), 7.132 (3.52), | |
| | 7.139 (2.90), 7.144 (1.97), 7.149 (2.55), 7.163 (0.69), 7.396 (1.86), | |
| | 7.412 (1.62), 7.417 (1.51), 7.532 (1.77), 7.536 (1.80), 7.553 (1.73), | |
| | 8.041 (1.32), 8.063 (16.00), 8.078 (0.76), 9.200 (0.86), 12.262 | |
| | (2.29). | |
| 168 | 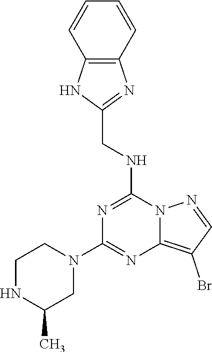 [SMILES: C[C@@H]1CN(c2nc(NCc3nc4ccccc4[nH]3)n3ncc(Br)c3n2)CCN1] | Intermediate 9 and CAS- RN: [75336- 86-6] 59% LC-MS (Method 2): Rₜ = 0.93 min; MS (ESIpos): m/z = 442 [M + H]⁺ Specific Optical Rotation (Method O1): +23.8° (c = 10 mg/mL, DMSO) |
| | N-[(1H-benzimidazol-2-yl)methyl]-8-bromo-2-[(3R)-3- | |
| | methylpiperazin-1-yl]pyrazolo[1,5-a][1,3,5]triazin-4-amine | |
| | ¹H-NMR (400 MHz, DMSO-d6) δ [ppm]: −0.008 (2.16), 0.008 (2.14), | |
| | 0.854 (0.63), 1.234 (0.44), 1.901 (1.24), 2.293 (0.78), 2.320 (1.04), | |
| | 2.325 (1.60), 2.329 (2.02), 2.334 (1.55), 2.339 (0.97), 2.521 (5.49), | |
| | 2.525 (3.59), 2.542 (1.99), 2.662 (0.87), 2.667 (1.51), 2.671 (1.92), | |
| | 2.676 (1.58), 2.681 (1.04), 4.362 (0.61), 4.849 (4.86), 7.096 (0.46), | |
| | 7.109 (1.87), 7.114 (1.48), 7.120 (2.23), 7.126 (2.57), 7.132 (2.48), | |
| | 7.137 (1.55), 7.142 (2.02), 7.156 (0.53), 7.387 (1.51), 7.403 (1.31), | |
| | 7.524 (1.41), 7.540 (1.34), 8.006 (16.00), 12.270 (1.77). | |
| 169 | 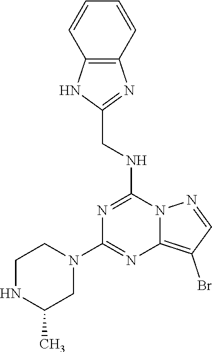 [SMILES: C[C@H]1CN(c2nc(NCc3nc4ccccc4[nH]3)n3ncc(Br)c3n2)CCN1] | Intermediate 9 and CAS- RN: [74879- 18-8] 55% LC-MS (Method 2): Rₜ = 0.93 min; MS (ESIpos): m/z = 442 [M + H]⁺ Specific Optical Rotation (Method O1): −25.0° (c = 10 mg/mL, DMSO) |
| | N-[(1H-benzimidazol-2-yl)methyl]-8-bromo-2-[(3S)-3- | |
| | methylpiperazin-1-yl]pyrazolo[1,5-a][1,3,5]triazin-4-amine | |
| | ¹H-NMR (400 MHz, DMSO-d6) δ [ppm]: −0.008 (1.26), 0.008 (1.26), | |
| | 0.853 (0.65), 1.235 (0.42), 2.077 (1.97), 2.320 (0.91), 2.325 (1.26), | |
| | 2.329 (1.52), 2.334 (1.22), 2.339 (0.85), 2.521 (3.63), 2.525 (2.47), | |
| | 2.542 (2.56), 2.662 (0.76), 2.667 (1.15), 2.671 (1.40), 2.676 (1.18), | |
| | 2.681 (0.88), 2.783 (0.48), 4.364 (0.62), 4.850 (5.29), 7.096 (0.42), | |
| | 7.110 (1.74), 7.120 (2.28), 7.126 (2.29), 7.133 (2.51), 7.142 (1.95), | |
| | 7.156 (0.45), 7.387 (1.36), 7.404 (1.19), 7.524 (1.32), 7.541 (1.22), | |
| | 8.006 (16.00), 8.021 (0.47), 12.266 (1.61). | |
| 170 | 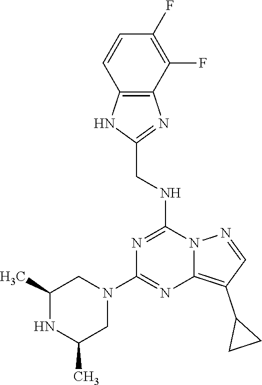 [SMILES: C[C@@H]1CN(c2nc(NCc3nc4c(F)c(F)ccc4[nH]3)n3ncc(C4CC4)c3n2)C[C@H](C)N1] | Intermediate 115 and CAS- RN: [21655- 48-1] 39% LC-MS (Method 2): Rₜ = 1.09 min; MS (ESIpos): m/z = 454 [M + H]⁺ |
| | 8-cyclopropyl-N-[(4,5-difluoro-1H-benzimidazol-2-yl)methyl]-2- | |
| | [(3R,5S)-3,5-dimethylpiperazin-1-yl]pyrazolo[1,5-a][1,3,5]triazin- | |
| | 4-amine | |
| | ¹H-NMR (400 MHz, DMSO-d6) δ [ppm]: −0.008 (2.87), 0.008 (2.58), | |
| | 0.692 (1.71), 0.698 (1.56), 0.703 (4.61), 0.709 (5.66), 0.716 (6.60), | |
| | 0.721 (5.73), 0.730 (3.08), 0.751 (1.56), 0.766 (3.45), 0.773 (5.73), | |
| | 0.779 (4.50), 0.787 (4.32), 0.791 (3.41), 0.795 (6.86), 0.800 (5.12), | |
| | 0.804 (3.08), 0.811 (3.56), 0.844 (3.41), 1.038 (0.94), 1.055 (2.03), | |
| | 1.073 (1.05), 1.701 (0.91), 1.714 (1.71), 1.722 (1.74), 1.728 (0.91), | |
| | 1.734 (3.08), 1.743 (1.09), 1.748 (1.67), 1.756 (1.60), 1.769 (0.76), | |
| | 2.099 (1.38), 2.320 (0.91), 2.325 (1.78), 2.329 (2.36), 2.334 (1.78), | |
| | 2.339 (0.98), 2.521 (7.62), 2.525 (5.19), 2.542 (6.02), 2.662 (0.76), | |
| | 2.667 (1.60), 2.671 (2.18), 2.676 (1.60), 2.681 (0.73), 4.276 (0.73), | |
| | 4.795 (4.61), 4.805 (4.57), 5.761 (1.02), 7.134 (0.69), 7.156 (1.67), | |
| | 7.174 (1.78), 7.184 (2.18), 7.201 (2.10), 7.675 (16.00), 8.847 (1.41). | |
| 171 | 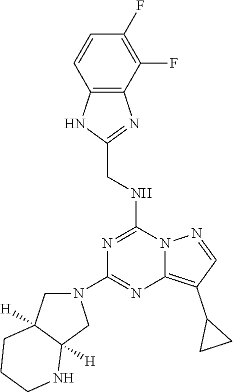 [SMILES: Fc1ccc2[nH]c(CNc3nc(N4C[C@@H]5CCCN[C@@H]5C4)nc4c(C5CC5)cnn34)nc2c1F] | Intermediate 115 and CAS- RN: [151213- 40-0] 39% LC-MS (Method 2): Rₜ = 1.13 min; MS (ESIpos): m/z = 466 [M + H]⁺ |
| | 8-cyclopropyl-N-[(4,5-difluoro-1H-benzimidazol-2-yl)methyl]-2- | |
| | [(4aS,7aS)-octahydro-6H-pyrrolo[3,4-b]pyridin-6-yl]pyrazolo[1,5- | |
| | a][1,3,5]triazin-4-amine | |
| | ¹H-NMR (400 MHz, DMSO-d6) δ [ppm]: −0.008 (2.17), 0.008 (2.04), | |
| | 0.726 (5.89), 0.732 (5.44), 0.763 (3.82), 0.770 (3.60), 0.781 (4.15), | |
| | 1.055 (0.71), 1.318 (1.55), 1.387 (0.87), 1.560 (2.20), 1.607 (0.81), | |
| | 1.652 (1.04), 1.685 (0.74), 1.698 (1.26), 1.719 (1.98), 1.731 (1.91), | |
| | 1.740 (1.30), 1.753 (1.13), 2.077 (0.84), 2.146 (0.74), 2.225 (0.68), | |
| | 2.339 (0.62), 2.521 (7.06), 2.525 (4.70), 2.542 (16.00), 2.787 (1.39), | |
| | 3.168 (2.59), 3.201 (2.20), 3.223 (3.63), 3.376 (4.99), 3.406 (4.73), | |
| | 3.428 (2.33), 3.467 (1.39), 3.488 (1.39), 3.515 (0.91), 4.840 (5.12), | |
| | 5.761 (2.79), 7.145 (1.13), 7.166 (2.27), 7.173 (1.46), 7.185 (2.36), | |
| | 7.195 (2.49), 7.213 (2.62), 7.237 (1.39), 7.654 (6.35), 7.659 (5.60), | |
| | 7.692 (0.52), 8.762 (1.59). | |
| 172 | 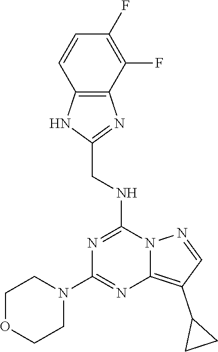 [SMILES: Fc1ccc2[nH]c(CNc3nc(N4CCOCC4)nc4c(C5CC5)cnn34)nc2c1F] | Intermediate 115 and CAS- RN: [110-91-8] 40% LC-MS (Method 2): Rₜ = 1.11 min; MS (ESIpos): m/z = 427 [M + H]⁺ |
| | 8-cyclopropyl-N-[(4,5-difluoro-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: −0.008 (2.19), 0.008 (1.87), | |
| | 0.709 (1.80), 0.721 (5.53), 0.726 (7.03), 0.734 (7.85), 0.739 (7.20), | |
| | 0.747 (3.24), 0.756 (1.28), 0.765 (1.90), 0.769 (3.50), 0.776 (6.25), | |
| | 0.781 (4.97), 0.790 (4.19), 0.797 (6.71), 0.802 (4.25), 0.809 (1.28), | |
| | 0.814 (1.77), 1.708 (1.05), 1.720 (1.96), 1.729 (2.09), 1.741 (3.27), | |
| | 1.749 (1.34), 1.755 (2.13), 1.762 (1.87), 1.775 (0.85), 2.334 (1.31), | |
| | 2.520 (7.56), 2.525 (4.84), 2.542 (6.35), 2.676 (1.37), 3.161 (0.49), | |
| | 3.174 (0.49), 3.486 (8.80), 3.552 (9.16), 3.563 (10.18), 4.840 (7.56), | |
| | 4.854 (7.53), 5.761 (1.44), 7.151 (0.75), 7.173 (1.90), 7.191 (2.36), | |
| | 7.201 (3.11), 7.215 (2.91), 7.715 (16.00), 8.927 (1.57), 12.664 | |
| | (1.28). | |
| 173 | 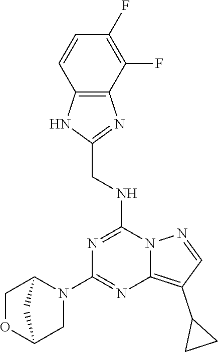 [SMILES: Fc1ccc2[nH]c(CNc3nc(N4C[C@@H]5C[C@H]4CO5)nc4c(C5CC5)cnn34)nc2c1F] | Intermediate 115 and CAS- RN: [31560- 06-2] 35% LC-MS (Method 2): Rₜ = 1.05 min; MS (ESIpos): m/z = 439 [M + H]⁺ |
| | 8-cyclopropyl-N-[(4,5-difluoro-1H-benzimidazol-2-yl)methyl]-2- | |
| | [(1S,4S)-2-oxa-5-azabicyclo[2.2.1]heptan-5-yl]pyrazolo[1,5- | |
| | a][1,3,5]triazin-4-amine | |
| | ¹H-NMR (400 MHz, DMSO-d6) δ [ppm]: −0.008 (1.71), 0.008 (1.41), | |
| | 0.700 (1.38), 0.712 (4.53), 0.717 (5.91), 0.724 (5.97), 0.730 (5.83), | |
| | 0.738 (2.57), 0.758 (1.30), 0.772 (4.15), 0.793 (3.95), 1.732 (2.02), | |
| | 1.753 (1.60), 1.776 (1.93), 2.325 (1.16), 2.330 (1.60), 2.334 (1.16), | |
| | 2.525 (4.01), 2.542 (2.65), 2.668 (1.19), 2.672 (1.60), 2.676 (1.13), | |
| | 3.168 (0.97), 3.189 (0.94), 4.538 (4.89), 4.659 (0.94), 4.834 (5.58), | |
| | 5.762 (3.56), 7.146 (0.83), 7.168 (1.85), 7.174 (1.16), 7.186 (1.96), | |
| | 7.196 (2.18), 7.213 (2.49), 7.691 (16.00), 8.908 (0.50). | |
| 174 | 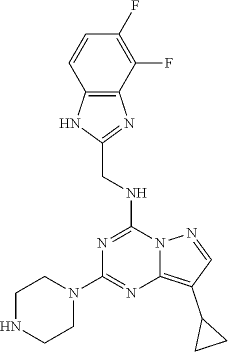 [SMILES: Fc1ccc2[nH]c(CNc3nc(N4CCNCC4)nc4c(C5CC5)cnn34)nc2c1F] | Intermediate 115 and CAS- RN: [110-85-0] 26% LC-MS (Method 2): Rₜ = 0.93 min; MS (ESIpos): m/z = 426 [M + H]⁺ |
| | 8-cyclopropyl-N-[(4,5-difluoro-1H-benzimidazol-2-yl)methyl]-2- | |
| | (piperazin-1-yl)pyrazolo[1,5-a][1,3,5]triazin-4-amine | |
| | ¹H-NMR (400 MHz, DMSO-d6) δ [ppm]: −0.008 (2.50), 0.008 (1.97), | |
| | 0.703 (1.73), 0.715 (5.60), 0.720 (7.13), 0.727 (7.57), 0.733 (7.47), | |
| | 0.740 (3.07), 0.751 (1.23), 0.764 (3.30), 0.771 (6.17), 0.776 (4.80), | |
| | 0.784 (4.13), 0.791 (6.47), 0.797 (4.20), 0.804 (1.47), 0.809 (1.63), | |
| | 1.038 (1.63), 1.055 (3.27), 1.073 (1.70), 1.236 (0.50), 1.698 (1.00), | |
| | 1.711 (1.97), 1.719 (2.03), 1.732 (3.20), 1.741 (1.40), 1.745 (1.87), | |
| | 1.753 (1.80), 1.766 (0.83), 2.291 (0.67), 2.326 (1.57), 2.329 (2.03), | |
| | 2.334 (1.53), 2.525 (5.13), 2.542 (1.50), 2.590 (6.00), 2.668 (1.63), | |
| | 2.671 (2.07), 2.676 (1.53), 3.370 (5.67), 3.413 (4.50), 3.431 (4.57), | |
| | 3.448 (3.90), 3.466 (2.50), 4.827 (9.70), 5.762 (1.33), 5.977 (0.80), | |
| | 7.147 (0.87), 7.168 (1.90), 7.186 (1.97), 7.197 (2.13), 7.214 (2.33), | |
| | 7.393 (0.83), 7.693 (16.00), 8.875 (0.80). | |
| 175 | 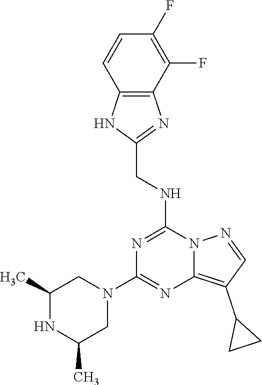 [SMILES: C[C@@H]1CN(c2nc(NCc3nc4c(F)c(F)ccc4[nH]3)n3ncc(C4CC4)c3n2)C[C@H](C)N1] | Intermediate 121 and CAS- RN: [21655- 48-1] 13% LC-MS (Method 2): Rₜ = 1.07 min; MS (ESIpos): m/z = 436 [M + H]⁺ |
| | 8-cyclopropyl-2-[(3R,5S)-3,5-dimethylpiperazin-1-yl]-N-[(5- | |
| | fluoro-1H-benzimidazol-2-yl)methyl]pyrazolo[1,5-a][1,3,5]triazin- | |
| | 4-amine | |
| | ¹H-NMR (400 MHz, DMSO-d6) δ [ppm]: −0.009 (2.40), 0.007 (3.71), | |
| | 0.673 (0.51), 0.692 (1.81), 0.704 (5.26), 0.709 (6.78), 0.716 (7.49), | |
| | 0.721 (6.82), 0.730 (3.37), 0.752 (1.56), 0.768 (3.71), 0.775 (6.40), | |
| | 0.780 (5.22), 0.788 (4.84), 0.796 (7.54), 0.801 (5.68), 0.813 (3.45), | |
| | 0.857 (5.64), 0.987 (1.73), 1.002 (2.78), 1.017 (1.60), 1.704 (1.01), | |
| | 1.716 (1.94), 1.724 (2.06), 1.730 (1.35), 1.737 (3.49), 1.745 (1.39), | |
| | 1.750 (2.06), 1.758 (1.89), 1.771 (0.84), 2.126 (2.40), 2.325 (1.98), | |
| | 2.329 (2.69), 2.333 (2.23), 2.524 (9.39), 2.542 (13.35), 2.667 (1.94), | |
| | 2.671 (2.65), 2.676 (2.06), 4.316 (1.39), 4.799 (5.43), 5.760 (4.84), | |
| | 6.964 (1.47), 6.987 (1.64), 7.170 (0.88), 7.192 (0.93), 7.298 (1.05), | |
| | 7.323 (1.01), 7.358 (0.80), 7.370 (0.97), 7.390 (0.80), 7.506 (0.80), | |
| | 7.610 (0.80), 7.669 (16.00), 8.788 (1.68), 12.373 (1.98). | |
| 176 | 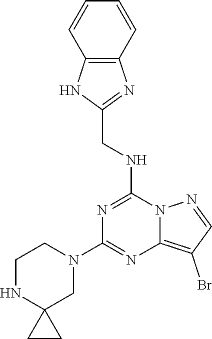 [SMILES: Brc1cnn2c(NCc3nc4ccccc4[nH]3)nc(N3CCNC4(CC4)C3)nc12] | Intermediate 9 and CAS- RN: [145122- 56-1] 16% LC-MS (Method 2): Rₜ = 0.96 min; MS (ESIpos): m/z = 454 [M + H]⁺ |
| | N-[(1H-benzimidazol-2-yl)methyl]-8-bromo-2-(4,7- | |
| | diazaspiro[2.5]octan-7-yl)pyrazolo[1,5-a][1,3,5]triazin-4-amine | |
| | ¹H-NMR (400 MHz, DMSO-d6) δ [ppm]: −0.008 (2.50), 0.008 (2.13), | |
| | 0.146 (0.88), 0.854 (0.64), 1.235 (3.28), 1.754 (0.64), 1.909 (0.58), | |
| | 2.291 (0.98), 2.325 (1.45), 2.329 (1.96), 2.334 (1.42), 2.520 (9.03), | |
| | 2.525 (5.95), 2.542 (6.22), 2.667 (2.16), 2.671 (2.67), 2.676 (2.03), | |
| | 3.631 (1.25), 4.788 (1.45), 7.095 (0.58), 7.109 (2.20), 7.119 (3.08), | |
| | 7.125 (3.32), 7.131 (3.45), 7.141 (2.67), 7.154 (0.81), 7.380 (1.62), | |
| | 7.397 (1.56), 7.510 (1.66), 7.527 (1.52), 8.001 (16.00), 8.014 (0.64), | |
| | 8.159 (0.81), 9.062 (1.86), 12.257 (2.16). | |
| 177 | 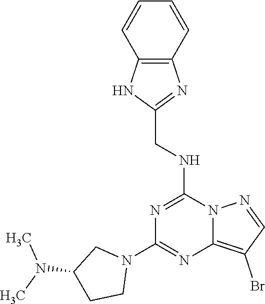 [SMILES: CN(C)[C@H]1CCN(c2nc(NCc3nc4ccccc4[nH]3)n3ncc(Br)c3n2)C1] | Intermediate 9 and CAS- RN: [132883- 44-4] 17% LC-MS (Method 2): Rₜ = 1.02 min; MS (ESIneg): m/z = 454 [M − H]⁻ |
| | N-[(1H-benzimidazol-2-yl)methyl]-8-bromo-2-[(3S)-3- | |
| | (dimethylamino)pyrrolidin-1-yl]pyrazolo[1,5-a][1,3,5]triazin-4- | |
| | amine | |
| | ¹H-NMR (400 MHz, DMSO-d6) δ [ppm]: −0.009 (0.56), 0.000 (16.00), | |
| | 0.007 (0.66), 0.835 (0.47), 0.853 (0.85), 1.234 (4.71), 1.663 (0.94), | |
| | 1.689 (1.04), 1.779 (0.66), 2.068 (11.86), 2.076 (7.72), 2.160 | |
| | (10.92), 2.294 (0.94), 2.325 (1.88), 2.329 (2.54), 2.334 (1.88), 2.525 | |
| | (7.25), 2.559 (0.94), 2.668 (2.35), 2.672 (3.11), 2.676 (2.45), 2.971 | |
| | (0.56), 2.998 (0.85), 3.019 (0.56), 3.101 (0.66), 3.126 (0.85), 3.151 | |
| | (0.66), 3.562 (1.22), 3.588 (1.51), 3.616 (1.04), 3.734 (0.56), 3.753 | |
| | (0.66), 3.778 (0.47), 4.863 (3.01), 7.128 (3.58), 7.158 (0.85), 7.399 | |
| | (1.79), 7.527 (1.79), 7.997 (8.28), 9.032 (1.41), 12.240 (1.04), | |
| | 12.291 (1.13). | |
| 178 | 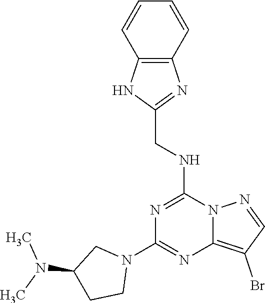 [SMILES: CN(C)[C@@H]1CCN(c2nc(NCc3nc4ccccc4[nH]3)n3ncc(Br)c3n2)C1] | Intermediate 9 and CAS- RN: [132958- 72-6] 13% LC-MS (Method 2): Rₜ = 1.01 min; MS (ESIneg): m/z = 454 [M − H]⁻ Specific Optical Rotation (Method O1): +27.0° (c = 10 mg/mL, DMSO) |
| | N-[(1H-benzimidazol-2-yl)methyl]-8-bromo-2-[(3R)-3- | |
| | (dimethylamino)pyrrolidin-1-yl]pyrazolo[1,5-a][1,3,5]triazin-4- | |
| | amine | |
| | ¹H-NMR (400 MHz, DMSO-d6) δ [ppm]: 0.000 (12.87), 0.835 (0.50), | |
| | 0.853 (0.86), 1.235 (5.00), 1.663 (1.36), 1.689 (1.46), 1.713 (0.96), | |
| | 1.822 (0.71), 2.069 (16.00), 2.076 (10.45), 2.126 (1.82), 2.162 | |
| | (14.99), 2.180 (3.08), 2.291 (0.81), 2.325 (2.27), 2.329 (3.03), 2.334 | |
| | (2.27), 2.525 (10.09), 2.667 (2.88), 2.672 (3.79), 2.676 (3.13), 2.974 | |
| | (0.76), 3.000 (1.11), 3.022 (0.86), 3.103 (0.86), 3.128 (1.16), 3.151 | |
| | (0.86), 3.562 (1.72), 3.589 (2.22), 3.616 (1.46), 3.736 (0.86), 3.754 | |
| | (0.96), 4.863 (4.24), 7.128 (5.05), 7.399 (2.47), 7.528 (2.57), 7.998 | |
| | (10.55), 8.214 (0.56), 9.036 (2.07), 12.236 (1.51), 12.286 (1.56). | |
| 179 | 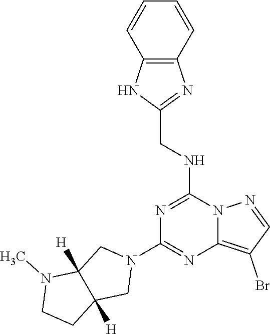 [SMILES: CN1CC[C@H]2CN(c3nc(NCc4nc5ccccc5[nH]4)n4ncc(Br)c4n3)C[C@H]21] | Intermediate 9 and CAS- RN: [1312756- 38-9] 43% LC-MS (Method 2): Rₜ = 1.08 min; MS (ESIpos): m/z = 468 [M + H]⁺ Specific Optical Rotation (Method O1): −13.3° (c = 10 mg/mL, DMSO) |
| | N-[(1H-benzimidazol-2-yl)methyl]-8-bromo-2-[(3aS,6aS)-1- | |
| | methylhexahydropyrrolo[3,4-b]pyrrol-5 (1H)-yl]pyrazolo[1,5- | |
| | a][1,3,5]triazin-4-amine | |
| | ¹H-NMR (400 MHz, DMSO-d6) δ [ppm]: 1.394 (0.52), 1.572 (0.55), | |
| | 1.906 (0.82), 1.996 (3.70), 2.122 (0.96), 2.143 (2.20), 2.164 (2.25), | |
| | 2.207 (3.32), 2.329 (1.51), 2.334 (1.15), 2.542 (2.06), 2.671 (2.31), | |
| | 2.676 (1.95), 2.736 (1.87), 2.894 (1.45), 2.915 (2.58), 2.935 (1.34), | |
| | 3.147 (0.69), 3.237 (1.34), 3.486 (0.82), 3.516 (0.74), 3.622 (1.48), | |
| | 3.654 (1.32), 4.854 (6.15), 4.868 (6.07), 7.094 (0.99), 7.118 (4.17), | |
| | 7.124 (5.19), 7.130 (4.45), 7.140 (3.57), 7.154 (1.10), 7.392 (3.40), | |
| | 7.410 (2.94), 7.526 (3.13), 7.543 (2.96), 7.990 (16.00), 8.004 (0.63), | |
| | 9.019 (2.36), 12.258 (2.22). | |
| 180 | 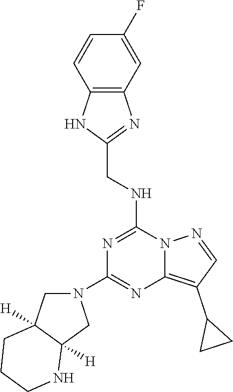 [SMILES: Fc1ccc2[nH]c(CNc3nc(N4C[C@@H]5CCCN[C@@H]5C4)nc4c(C5CC5)cnn34)nc2c1] | Intermediate 121 and CAS- RN: [151213- 40-0] 24% LC-MS (Method 2): Rₜ = 1.12 min; MS (ESIpos): m/z = 448 [M + H]⁺ |
| | 8-cyclopropyl-N-[(5-fluoro-1H-benzimidazol-2-yl)methyl]-2- | |
| | [(4aS, 7aS)-octahydro-6H-pyrrolo[3,4-b]pyridin-6-yl]pyrazolo[1,5- | |
| | a][1,3,5]triazin-4-amine | |
| | ¹H-NMR (400 MHz, DMSO-d6) δ [ppm]: −0.149 (0.69), −0.008 (6.51), | |
| | 0.008 (5.89), 0.146 (0.69), 0.726 (9.63), 0.751 (4.71), 0.763 (6.65), | |
| | 0.769 (6.23), 0.779 (7.00), 1.235 (0.55), 1.306 (2.29), 1.346 (2.35), | |
| | 1.562 (3.53), 1.652 (1.73), 1.685 (1.66), 1.698 (2.35), 1.719 (3.46), | |
| | 1.732 (3.19), 1.740 (2.22), 1.753 (2.08), 2.102 (1.94), 2.402 (2.15), | |
| | 2.428 (3.81), 2.452 (2.77), 2.520 (16.00), 2.525 (9.97), 2.542 | |
| | (13.37), 2.735 (3.81), 2.764 (3.19), 2.997 (0.42), 3.141 (2.56), 3.170 | |
| | (3.12), 3.239 (1.45), 3.265 (3.05), 3.292 (3.12), 3.389 (5.54), 3.417 | |
| | (3.81), 3.441 (2.42), 3.452 (2.35), 3.472 (2.29), 3.499 (0.97), 4.785 | |
| | (0.62), 4.840 (10.87), 5.761 (1.25), 6.954 (3.12), 6.960 (3.53), 6.978 | |
| | (4.78), 6.981 (5.19), 7.000 (3.46), 7.006 (3.74), 7.258 (2.70), 7.277 | |
| | (2.63), 7.450 (2.56), 7.643 (13.23), 8.671 (1.73), 12.329 (0.62). | |
| 181 | 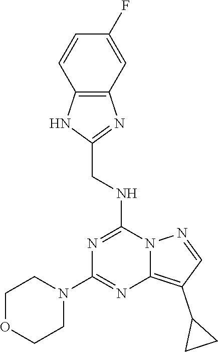 [SMILES: Fc1ccc2[nH]c(CNc3nc(N4CCOCC4)nc4c(C5CC5)cnn34)nc2c1] | Intermediate 121 and CAS- RN: [110-91-8] 15% LC-MS (Method 2): Rₜ = 1.10 min; MS (ESIpos): m/z = 409 [M + H]⁺ |
| | 8-cyclopropyl-N-[(5-fluoro-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: −0.008 (1.97), 0.008 (1.84), | |
| | 0.709 (1.54), 0.712 (0.99), 0.721 (4.80), 0.726 (6.29), 0.733 (7.18), | |
| | 0.738 (6.62), 0.747 (3.01), 0.756 (1.06), 0.765 (1.43), 0.770 (2.98), | |
| | 0.777 (5.43), 0.782 (4.15), 0.790 (3.74), 0.794 (2.57), 0.797 (5.79), | |
| | 0.803 (3.74), 0.809 (1.12), 0.815 (1.58), 1.709 (0.93), 1.721 (1.77), | |
| | 1.729 (1.82), 1.742 (2.92), 1.750 (1.12), 1.755 (1.73), 1.763 (1.69), | |
| | 1.776 (0.76), 2.521 (4.63), 2.525 (3.16), 2.542 (5.66), 3.491 (7.16), | |
| | 3.560 (7.31), 3.571 (8.09), 4.824 (4.69), 4.837 (4.69), 5.761 (6.57), | |
| | 6.974 (1.04), 6.996 (1.12), 7.194 (0.56), 7.217 (0.58), 7.315 (0.67), | |
| | 7.339 (0.65), 7.389 (0.65), 7.517 (0.56), 7.710 (16.00), 8.871 (1.77), | |
| | 12.355 (1.64). | |
| 182 | 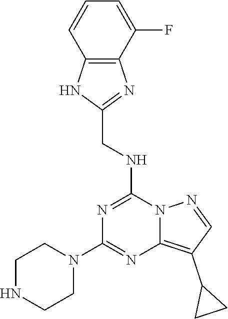 [SMILES: Fc1cccc2[nH]c(CNc3nc(N4CCNCC4)nc4c(C5CC5)cnn34)nc12] | Intermediate 123 and CAS- RN: [110-85-0] 9% LC-MS (Method 2): Rₜ = 0.96 min; MS (ESIpos): m/z = 408 [M + H]⁺ |
| | 8-cyclopropyl-N-[(4-fluoro-1H-benzimidazol-2-yl)methyl]-2- | |
| | (piperazin-1-yl)pyrazolo[1,5-a][1,3,5]triazin-4-amine | |
| | ¹H-NMR (400 MHz, DMSO-d6) δ [ppm]: −0.008 (0.96), 0.008 (0.84), | |
| | 0.703 (1.27), 0.716 (3.80), 0.721 (4.92), 0.728 (5.63), 0.733 (4.64), | |
| | 0.741 (2.38), 0.749 (0.91), 0.759 (1.37), 0.762 (2.31), 0.769 (4.03), | |
| | 0.774 (3.73), 0.782 (2.81), 0.787 (2.05), 0.790 (4.49), 0.795 (2.99), | |
| | 0.802 (0.86), 0.807 (1.27), 1.234 (0.46), 1.698 (0.66), 1.711 (1.32), | |
| | 1.718 (1.37), 1.731 (2.18), 1.740 (0.86), 1.744 (1.29), 1.753 (1.77), | |
| | 1.765 (0.61), 1.905 (0.58), 2.334 (1.06), 2.338 (0.48), 2.521 (5.73), | |
| | 2.525 (4.29), 2.542 (16.00), 2.676 (1.12), 3.514 (5.27), 4.834 (6.57), | |
| | 5.761 (5.00), 6.936 (0.84), 6.958 (0.66), 7.087 (1.12), 7.099 (1.27), | |
| | 7.106 (2.18), 7.119 (2.31), 7.127 (1.32), 7.139 (1.24), 7.236 (0.79), | |
| | 7.684 (10.98), 8.826 (0.41). | |
| 183 | 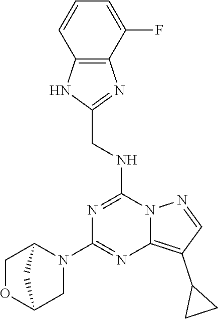 [SMILES: Fc1cccc2[nH]c(CNc3nc(N4C[C@@H]5C[C@H]4CO5)nc4c(C5CC5)cnn34)nc12] | Intermediate 123 and CAS- RN: [31560- 06-2] 9% LC-MS (Method 2): Rₜ = 1.04 min; MS (ESIpos): m/z = 421 [M + H]⁺ |
| | 8-cyclopropyl-N-[(4-fluoro-1H-benzimidazol-2-yl)methyl]-2- | |
| | [(1S,4S)-2-oxa-5-azabicyclo[2.2.1]heptan-5-yl]pyrazolo[1,5- | |
| | a][1,3,5]triazin-4-amine | |
| | ¹H-NMR (400 MHz, DMSO-d6) δ [ppm]: −0.008 (3.48), 0.008 (3.48), | |
| | 0.719 (7.23), 0.726 (7.67), 0.731 (7.06), 0.738 (3.09), 0.773 (5.46), | |
| | 0.793 (5.41), 1.067 (0.44), 1.234 (1.16), 1.732 (2.65), 1.753 (2.54), | |
| | 1.775 (2.59), 2.324 (2.32), 2.329 (3.20), 2.333 (2.32), 2.525 (8.77), | |
| | 2.542 (6.62), 2.667 (2.37), 2.671 (3.20), 2.676 (2.32), 3.189 (0.99), | |
| | 3.672 (0.66), 4.534 (6.40), 4.670 (1.32), 4.835 (5.57), 5.761 (8.11), | |
| | 6.904 (2.15), 6.923 (2.92), 6.931 (2.43), 6.951 (2.70), 6.974 (0.50), | |
| | 6.995 (0.72), 7.021 (0.61), 7.087 (1.82), 7.100 (1.88), 7.108 (3.59), | |
| | 7.120 (3.26), 7.128 (2.10), 7.139 (1.66), 7.229 (4.41), 7.248 (3.26), | |
| | 7.365 (0.94), 7.384 (0.83), 7.682 (3.31), 7.691 (16.00), 8.894 (1.38), | |
| | 12.543 (3.64), 12.890 (0.77). | |
| 184 | 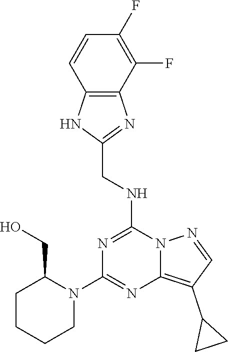 [SMILES: OC[C@@H]1CCCCN1c1nc(NCc2nc3c(F)c(F)ccc3[nH]2)n2ncc(C3CC3)c2n1] | Intermediate 115 and CAS- RN: [41373- 39-1] 8% LC-MS (Method 2): Rₜ = 1.19 min; MS (ESIpos): m/z = 455 [M + H]⁺ |
| | [(2S)-1-(8-cyclopropyl-4-{[(4,5-difluoro-1H-benzimidazol-2- | |
| | yl)methyl]amino}pyrazolo[1,5-a][1,3,5]triazin-2-yl)piperidin-2- | |
| | yl]methanol | |
| | ¹H-NMR (400 MHz, DMSO-d6) δ [ppm]: −0.149 (0.42), −0.009 (4.17), | |
| | 0.007 (3.75), 0.146 (0.48), 0.706 (1.64), 0.718 (4.65), 0.723 (6.13), | |
| | 0.730 (7.34), 0.735 (6.13), 0.744 (2.75), 0.749 (1.32), 0.761 (3.59), | |
| | 0.768 (5.70), 0.773 (4.22), 0.782 (3.38), 0.785 (2.75), 0.789 (6.13), | |
| | 0.794 (3.85), 0.806 (1.48), 1.144 (0.69), 1.156 (0.69), 1.174 (0.74), | |
| | 1.192 (0.53), 1.234 (0.79), 1.324 (1.11), 1.339 (1.00), 1.355 (0.90), | |
| | 1.484 (2.01), 1.517 (1.69), 1.549 (1.16), 1.697 (1.16), 1.710 (1.95), | |
| | 1.717 (2.01), 1.731 (3.17), 1.739 (1.80), 1.743 (2.69), 1.752 (3.06), | |
| | 1.764 (1.48), 1.782 (1.16), 1.989 (0.48), 2.338 (0.95), 2.520 (11.14), | |
| | 2.524 (7.29), 2.542 (7.82), 2.676 (2.27), 2.680 (1.00), 2.764 (0.90), | |
| | 3.492 (1.16), 4.486 (0.58), 4.702 (0.95), 4.762 (1.27), 4.826 (2.96), | |
| | 4.840 (3.75), 5.760 (6.71), 7.149 (0.53), 7.172 (1.32), 7.189 (1.64), | |
| | 7.200 (2.17), 7.215 (2.64), 7.664 (16.00), 7.691 (0.48), 8.796 (1.21), | |
| | 12.567 (0.48). | |
| 185 | 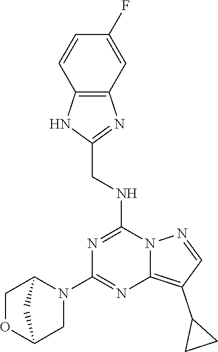 [SMILES: Fc1ccc2[nH]c(CNc3nc(N4C[C@@H]5C[C@H]4CO5)nc4c(C5CC5)cnn34)nc2c1] | Intermediate 121 and CAS- RN: [31560- 06-2] 15% LC-MS (Method 2): Rₜ = 1.04 min; MS (ESIpos): m/z = 421 [M + H]⁺ |
| | 8-cyclopropyl-N-[(5-fluoro-1H-benzimidazol-2-yl)methyl]-2- | |
| | [(1S,4S)-2-oxa-5-azabicyclo[2.2.1]heptan-5-yl]pyrazolo[1,5- | |
| | a][1,3,5]triazin-4-amine | |
| | ¹H-NMR (400 MHz, DMSO-d6) δ [ppm]: −0.008 (2.18), 0.008 (2.08), | |
| | 0.699 (1.24), 0.703 (1.07), 0.711 (3.89), 0.716 (5.28), 0.723 (5.52), | |
| | 0.728 (5.33), 0.737 (2.33), 0.757 (1.14), 0.772 (3.59), 0.786 (2.50), | |
| | 0.793 (3.42), 1.733 (1.66), 1.753 (1.51), 1.776 (1.49), 1.990 (0.42), | |
| | 2.291 (0.40), 2.339 (0.45), 2.521 (4.85), 2.525 (3.32), 2.542 (2.85), | |
| | 2.681 (0.47), 2.933 (0.87), 3.652 (1.04), 3.664 (1.26), 3.675 (1.04), | |
| | 4.538 (3.74), 4.673 (0.72), 4.817 (3.44), 5.761 (9.96), 6.943 (0.42), | |
| | 6.966 (1.16), 6.991 (1.26), 7.012 (0.50), 7.187 (0.64), 7.208 (0.64), | |
| | 7.309 (0.79), 7.334 (0.82), 7.380 (0.69), 7.502 (0.59), 7.515 (0.67), | |
| | 7.535 (0.52), 7.685 (16.00), 8.850 (0.84), 12.353 (1.14). | |
| 186 | 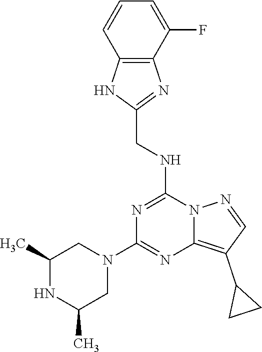 [SMILES: C[C@@H]1CN(c2nc(NCc3nc4c(F)cccc4[nH]3)n3ncc(C4CC4)c3n2)C[C@H](C)N1] | Intermediate 123 and CAS- RN: [21655- 48-1] 16% LC-MS (Method 2): Rₜ = 1.06 min; MS (ESIpos): m/z = 436 [M + H]⁺ |
| | 8-cyclopropyl-2-[(3R,5S)-3,5-dimethylpiperazin-1-yl]-N-[(4- | |
| | fluoro-1H-benzimidazol-2-yl)methyl]pyrazolo[1,5-a][1,3,5]triazin- | |
| | 4-amine | |
| | ¹H-NMR (400 MHz, DMSO-d6) δ [ppm]: −0.008 (3.22), 0.008 (2.84), | |
| | 0.673 (0.47), 0.681 (0.47), 0.686 (0.57), 0.693 (1.89), 0.700 (1.61), | |
| | 0.705 (5.02), 0.711 (6.15), 0.718 (7.20), 0.723 (6.15), 0.732 (3.41), | |
| | 0.752 (1.70), 0.767 (4.17), 0.775 (6.53), 0.780 (4.83), 0.788 (4.64), | |
| | 0.792 (3.69), 0.796 (7.38), 0.801 (5.30), 0.806 (3.03), 0.813 (3.60), | |
| | 0.851 (3.98), 0.987 (1.61), 1.002 (2.46), 1.017 (1.51), 1.234 (0.47), | |
| | 1.704 (0.95), 1.716 (1.99), 1.724 (1.99), 1.730 (1.04), 1.737 (3.41), | |
| | 1.745 (1.23), 1.750 (1.80), 1.758 (1.80), 1.771 (0.76), 2.108 (1.61), | |
| | 2.339 (1.14), 2.521 (7.86), 2.526 (5.21), 2.543 (3.03), 2.681 (0.85), | |
| | 4.297 (0.95), 4.807 (4.92), 4.816 (4.92), 5.760 (0.95), 6.926 (1.04), | |
| | 6.949 (0.85), 7.078 (1.51), 7.090 (1.61), 7.099 (2.93), 7.111 (3.12), | |
| | 7.118 (1.80), 7.130 (1.70), 7.215 (1.14), 7.232 (0.95), 7.610 (0.85), | |
| | 7.673 (16.00), 8.826 (1.14), 12.568 (0.76). | |
| 187 | 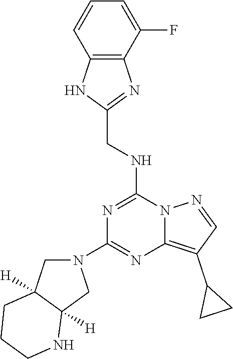 [SMILES: Fc1cccc2[nH]c(CNc3nc(N4C[C@@H]5CCCN[C@@H]5C4)nc4c(C5CC5)cnn34)nc12] | Intermediate 123 and CAS- RN: [151213- 40-0] 15% LC-MS (Method 2): Rₜ = 1.10 min; MS (ESIpos): m/z = 448 [M + H]⁺ |
| | 8-cyclopropyl-N-[(4-fluoro-1H-benzimidazol-2-yl)methyl]-2- | |
| | [(4aS,7aS)-octahydro-6H-pyrrolo[3,4-b]pyridin-6-yl]pyrazolo[1,5- | |
| | a][1,3,5]triazin-4-amine | |
| | ¹H-NMR (400 MHz, DMSO-d6) δ [ppm]: −0.009 (3.11), 0.007 (2.84), | |
| | 0.727 (4.76), 0.732 (4.87), 0.739 (4.11), 0.751 (3.03), 0.757 (3.26), | |
| | 0.762 (3.38), 0.770 (3.49), 0.777 (3.49), 0.782 (3.38), 1.233 (0.58), | |
| | 1.299 (1.19), 1.339 (1.30), 1.555 (1.92), 1.651 (0.92), 1.685 (0.88), | |
| | 1.699 (1.15), 1.704 (1.15), 1.719 (1.69), 1.731 (1.57), 1.753 (1.27), | |
| | 2.086 (0.92), 2.397 (0.96), 2.424 (1.92), 2.448 (1.88), 2.452 (2.00), | |
| | 2.457 (1.42), 2.462 (0.54), 2.520 (9.02), 2.524 (6.14), 2.534 (1.65), | |
| | 2.542 (16.00), 2.680 (0.81), 2.729 (1.73), 2.759 (1.46), 2.996 (0.54), | |
| | 3.138 (1.34), 3.167 (1.69), 3.224 (1.07), 3.251 (1.96), 3.277 (2.11), | |
| | 3.383 (3.72), 3.415 (2.38), 3.442 (1.57), 3.471 (1.34), 3.497 (0.61), | |
| | 4.842 (2.38), 4.863 (3.91), 5.760 (2.99), 6.912 (2.00), 6.932 (2.61), | |
| | 6.940 (2.19), 6.959 (2.46), 7.081 (2.11), 7.093 (2.26), 7.101 (4.07), | |
| | 7.113 (4.26), 7.121 (2.53), 7.133 (2.23), 7.258 (3.30), 7.278 (2.61), | |
| | 7.645 (6.48), 8.693 (0.73). | |
| 188 | 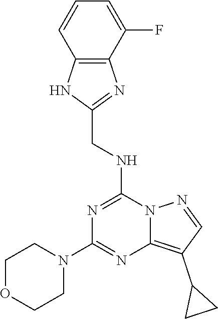 [SMILES: Fc1cccc2[nH]c(CNc3nc(N4CCOCC4)nc4c(C5CC5)cnn34)nc12] | Intermediate 123 and CAS- RN: [110-91-8] 19% LC-MS (Method 2): Rₜ = 1.09 min; MS (ESIpos): m/z = 409 [M − H]⁺ |
| | 8-cyclopropyl-N-[(4-fluoro-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: −0.008 (2.49), 0.008 (2.25), | |
| | 0.711 (1.85), 0.723 (5.89), 0.728 (7.35), 0.736 (8.74), 0.741 (7.10), | |
| | 0.749 (3.55), 0.757 (1.52), 0.766 (2.19), 0.769 (3.95), 0.776 (6.68), | |
| | 0.782 (5.86), 0.790 (4.74), 0.794 (3.07), 0.797 (7.26), 0.802 (4.71), | |
| | 0.810 (1.28), 0.815 (2.13), 1.235 (0.82), 1.709 (1.18), 1.722 (2.13), | |
| | 1.730 (2.25), 1.743 (3.49), 1.751 (1.34), 1.756 (2.13), 1.764 (2.00), | |
| | 1.777 (0.94), 2.339 (0.58), 2.521 (6.28), 2.525 (4.16), 2.542 (13.27), | |
| | 2.676 (1.28), 2.681 (0.55), 3.484 (8.20), 3.558 (8.93), 3.569 (9.81), | |
| | 4.847 (6.68), 4.861 (6.68), 6.912 (0.85), 6.932 (1.34), 6.959 (1.15), | |
| | 7.092 (1.37), 7.104 (1.64), 7.112 (2.79), 7.124 (2.94), 7.132 (1.67), | |
| | 7.144 (1.58), 7.236 (1.97), 7.255 (1.55), 7.714 (16.00), 8.910 (1.46), | |
| | 12.563 (1.43). | |
| 189 | 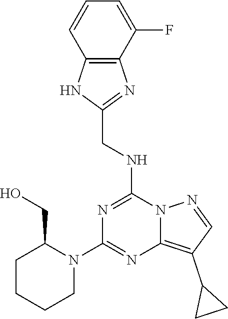 [SMILES: OC[C@@H]1CCCCN1c1nc(NCc2nc3c(F)cccc3[nH]2)n2ncc(C3CC3)c2n1] | Intermediate 123 and CAS- RN: [41373- 39-1] 5% LC-MS (Method 2): Rₜ = 1.16 min; MS (ESIpos): m/z = 437 [M + H]⁺ |
| | [(2S)-1-(8-cyclopropyl-4-{[(4-fluoro-1H-benzimidazol-2- | |
| | yl)methyl]amino}pyrazolo[1,5-a][1,3,5]triazin-2-yl)piperidin-2- | |
| | yl]methanol | |
| | ¹H-NMR (400 MHz, DMSO-d6) δ [ppm]: −0.008 (1.91), 0.008 (1.71), | |
| | 0.708 (1.49), 0.721 (4.62), 0.726 (5.88), 0.733 (6.85), 0.738 (5.98), | |
| | 0.746 (2.81), 0.750 (1.45), 0.763 (3.65), 0.769 (5.66), 0.775 (3.94), | |
| | 0.778 (2.07), 0.783 (3.30), 0.787 (2.78), 0.790 (6.08), 0.795 (3.68), | |
| | 0.807 (1.45), 1.068 (0.68), 1.145 (0.78), 1.157 (0.58), 1.175 (0.74), | |
| | 1.193 (0.58), 1.234 (0.68), 1.339 (1.03), 1.372 (0.71), 1.486 (2.04), | |
| | 1.520 (1.81), 1.551 (1.29), 1.700 (1.07), 1.713 (1.97), 1.720 (1.94), | |
| | 1.733 (3.01), 1.741 (1.49), 1.747 (2.33), 1.754 (3.04), 1.767 (1.71), | |
| | 1.790 (1.16), 1.990 (0.45), 2.339 (0.58), 2.521 (6.85), 2.525 (4.65), | |
| | 2.542 (16.00), 2.681 (0.61), 2.748 (0.58), 2.780 (0.91), 3.506 (1.65), | |
| | 4.497 (0.65), 4.725 (1.29), 4.791 (1.36), 4.805 (1.36), 4.836 (2.42), | |
| | 4.850 (3.85), 4.862 (2.29), 4.900 (0.45), 5.761 (5.27), 6.907 (1.00), | |
| | 6.928 (1.42), 6.935 (1.33), 6.955 (1.33), 7.091 (1.03), 7.103 (1.33), | |
| | 7.111 (2.07), 7.123 (2.20), 7.131 (1.23), 7.143 (1.10), 7.244 (2.36), | |
| | 7.264 (1.87), 7.662 (14.64), 8.778 (1.49), 12.466 (0.74). | |
| 190 | 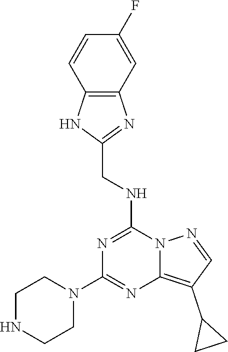 [SMILES: Fc1ccc2[nH]c(CNc3nc(N4CCNCC4)nc4c(C5CC5)cnn34)nc2c1] | Intermediate 121 and CAS- RN: [110-85-0] 5% LC-MS (Method 2): Rₜ = 0.97 min; MS (ESIpos): m/z = 408 [M + H]⁺ |
| | 8-cyclopropyl-N-[(5-fluoro-1H-benzimidazol-2-yl)methyl]-2- | |
| | (piperazin-1-yl)pyrazolo[1,5-a][1,3,5]triazin-4-amine | |
| | ¹H-NMR (400 MHz, DMSO-d6) δ [ppm]: −0.009 (1.58), 0.007 (1.46), | |
| | 0.700 (1.55), 0.712 (4.91), 0.717 (6.27), 0.725 (6.75), 0.730 (6.72), | |
| | 0.738 (2.79), 0.748 (1.01), 0.757 (1.46), 0.761 (2.79), 0.769 (5.26), | |
| | 0.774 (4.28), 0.782 (3.68), 0.790 (5.70), 0.795 (3.96), 0.801 (1.36), | |
| | 0.807 (1.55), 1.696 (0.86), 1.710 (1.68), 1.717 (1.81), 1.730 (2.82), | |
| | 1.739 (1.20), 1.743 (1.71), 1.752 (2.12), 1.764 (0.76), 2.324 (1.52), | |
| | 2.329 (2.00), 2.334 (1.49), 2.525 (5.54), 2.542 (16.00), 2.552 (6.18), | |
| | 2.666 (1.49), 2.671 (1.96), 2.676 (1.49), 2.730 (0.57), 3.512 (7.26), | |
| | 3.574 (0.60), 4.811 (7.45), 5.761 (6.34), 6.969 (1.17), 6.988 (1.24), | |
| | 7.209 (0.67), 7.311 (0.76), 7.336 (0.73), 7.384 (0.73), 7.518 (0.63), | |
| | 7.679 (14.32), 7.687 (1.43), 8.787 (0.73), 12.352 (0.95). | |
| 191 | 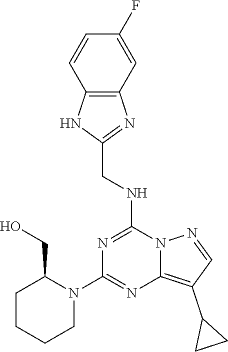 [SMILES: OC[C@@H]1CCCCN1c1nc(NCc2nc3cc(F)ccc3[nH]2)n2ncc(C3CC3)c2n1] | Intermediate 121 and CAS- RN: [41373- 39-1] 3% LC-MS (Method 2): Rₜ = 1.19 min; MS (ESIpos): m/z = 437 [M + H]⁺ |
| | [(2S)-1-(8-cyclopropyl-4-{[(5-fluoro-1H-benzimidazol-2- | |
| | yl)methyl]amino}pyrazolo[1,5-a][1,3,5]triazin-2-yl)piperidin-2- | |
| | yl]methanol | |
| | ¹H-NMR (400 MHz, DMSO-d6) δ [ppm]: −0.008 (2.17), 0.008 (2.08), | |
| | 0.705 (1.78), 0.717 (5.20), 0.721 (7.07), 0.729 (7.37), 0.734 (6.98), | |
| | 0.742 (3.04), 0.748 (1.47), 0.761 (3.60), 0.768 (6.42), 0.774 (4.94), | |
| | 0.782 (3.90), 0.790 (7.02), 0.795 (4.38), 0.806 (1.65), 1.232 (0.95), | |
| | 1.347 (1.30), 1.380 (1.08), 1.492 (2.95), 1.523 (2.34), 1.555 (1.82), | |
| | 1.697 (1.30), 1.711 (2.25), 1.718 (2.34), 1.731 (3.51), 1.740 (1.73), | |
| | 1.744 (2.60), 1.753 (3.47), 1.765 (2.34), 1.792 (1.47), 1.908 (0.48), | |
| | 2.334 (1.82), 2.520 (10.41), 2.525 (6.50), 2.542 (11.14), 2.676 | |
| | (1.91), 2.783 (1.26), 3.523 (2.60), 4.516 (0.87), 4.810 (3.86), 4.831 | |
| | (3.77), 4.845 (2.73), 4.872 (1.13), 5.761 (8.24), 6.951 (0.69), 6.976 | |
| | (1.73), 6.999 (1.91), 7.023 (0.78), 7.208 (1.17), 7.225 (1.17), 7.311 | |
| | (1.34), 7.335 (1.30), 7.387 (1.13), 7.399 (1.21), 7.409 (1.17), 7.421 | |
| | (1.08), 7.512 (1.04), 7.524 (1.13), 7.534 (1.04), 7.546 (0.91), 7.656 | |
| | (16.00), 7.892 (0.56), 8.730 (1.52), 8.744 (2.82), 8.758 (1.43), | |
| | 12.253 (0.87), 12.288 (1.08). | |
| 192 | 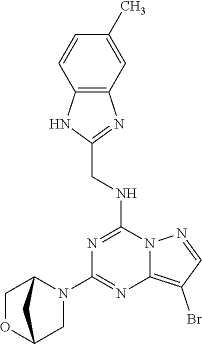 [SMILES: Cc1ccc2[nH]c(CNc3nc(N4C[C@H]5C[C@@H]4CO5)nc4c(Br)cnn34)nc2c1] | Intermediate 113 and CAS- RN: [601515- 79-1] 11% LC-MS (Method 2): Rₜ = 1.04 min; MS (ESIpos): m/z = 455 [M + H]⁺ |
| | 8-bromo-N-[(5-methyl-1H-benzimidazol-2-yl)methyl]-2-[(1R,4R)- | |
| | 2-oxa-5-azabicyclo[2.2.1]heptan-5-yl]pyrazolo[1,5- | |
| | a][1,3,5]triazin-4-amine | |
| | ¹H-NMR (400 MHz, DMSO-d6) δ [ppm]: 1.698 (0.58), 1.720 (0.96), | |
| | 1.751 (0.53), 1.783 (1.22), 1.808 (1.47), 2.331 (0.98), 2.371 (8.90), | |
| | 2.380 (10.82), 2.518 (4.36), 2.522 (2.89), 2.539 (2.05), 2.673 (0.96), | |
| | 3.275 (1.29), 3.381 (1.71), 3.409 (0.69), 3.690 (0.58), 3.763 (0.56), | |
| | 4.564 (3.38), 4.742 (1.38), 4.830 (3.20), 4.885 (0.82), 5.759 (10.50), | |
| | 6.923 (1.22), 6.945 (2.09), 6.968 (1.09), 7.179 (1.31), 7.256 (0.67), | |
| | 7.275 (0.71), 7.319 (1.69), 7.385 (1.34), 7.405 (1.22), 8.013 (16.00), | |
| | 9.089 (0.82), 12.071 (1.74), 12.095 (1.31). | |
| 193 | 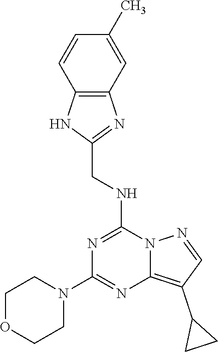 [SMILES: Cc1ccc2[nH]c(CNc3nc(N4CCOCC4)nc4c(C5CC5)cnn34)nc2c1] | Intermediate 146 and CAS- RN: [110-91-8] 11% LC-MS (Method 2): Rₜ = 1.10 min; MS (ESIpos): m/z = 405 [M + H]⁺ |
| | 8-cyclopropyl-N-[(5-methyl-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: 0.708 (1.74), 0.720 (4.91), | |
| | 0.725 (6.13), 0.733 (7.03), 0.738 (6.13), 0.746 (2.84), 0.755 (1.12), | |
| | 0.764 (1.67), 0.767 (3.15), 0.774 (5.53), 0.779 (4.48), 0.788 (3.72), | |
| | 0.792 (2.72), 0.795 (5.84), 0.801 (3.62), 0.808 (1.07), 0.812 (1.53), | |
| | 1.232 (1.07), 1.349 (0.52), 1.707 (0.91), 1.720 (1.79), 1.728 (1.81), | |
| | 1.741 (2.84), 1.749 (1.19), 1.754 (1.72), 1.762 (1.60), 1.775 (0.76), | |
| | 2.371 (9.92), 2.381 (12.40), 2.518 (5.27), 2.523 (3.58), 2.539 (1.50), | |
| | 3.502 (7.32), 3.570 (7.58), 3.581 (8.32), 3.648 (0.62), 3.660 (0.62), | |
| | 3.717 (0.41), 4.810 (5.10), 4.824 (5.15), 5.759 (1.98), 6.922 (1.50), | |
| | 6.944 (2.65), 6.965 (1.34), 7.186 (2.93), 7.262 (1.98), 7.282 (1.76), | |
| | 7.314 (2.34), 7.380 (2.38), 7.400 (2.19), 7.653 (0.52), 7.703 (16.00), | |
| | 8.816 (1.84), 8.824 (1.67), 12.072 (2.07), 12.098 (1.67). | |
| 194 | 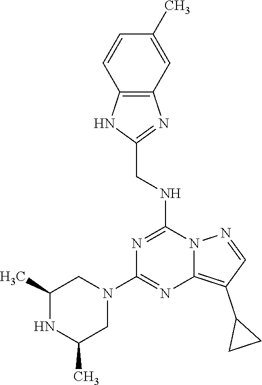 [SMILES: Cc1ccc2[nH]c(CNc3nc(N4C[C@@H](C)N[C@@H](C)C4)nc4c(C5CC5)cnn34)nc2c1] | Intermediate 146 and CAS- RN: [111-21- 91] 14% LC-MS (Method 2): Rₜ = 1.07 min; MS (ESIpos): m/z = 432 [M + H]⁺ |
| | 8-cyclopropyl-2-[(3R,5S)-3,5-dimethylpiperazin-1-yl]-N-[(5- | |
| | methyl-1H-benzimidazol-2-yl)methyl]pyrazolo[1,5- | |
| | a][1,3,5]triazin-4-amine | |
| | ¹H-NMR (400 MHz, DMSO-d6) δ [ppm]: 0.690 (1.55), 0.697 (1.48), | |
| | 0.702 (4.44), 0.707 (5.57), 0.715 (6.52), 0.720 (5.64), 0.728 (2.85), | |
| | 0.750 (1.19), 0.765 (2.90), 0.773 (5.22), 0.778 (3.85), 0.786 (3.49), | |
| | 0.790 (2.61), 0.794 (5.86), 0.799 (3.98), 0.810 (1.99), 0.879 (5.57), | |
| | 1.232 (1.10), 1.703 (0.88), 1.715 (1.72), 1.723 (1.77), 1.729 (0.93), | |
| | 1.736 (3.05), 1.744 (1.06), 1.749 (1.61), 1.757 (1.57), 1.770 (0.73), | |
| | 2.111 (1.24), 2.140 (2.17), 2.168 (1.48), 2.322 (1.06), 2.327 (1.44), | |
| | 2.332 (1.10), 2.367 (9.46), 2.377 (11.93), 2.518 (5.19), 2.523 (3.47), | |
| | 2.539 (1.04), 2.660 (0.46), 2.665 (0.95), 2.669 (1.30), 2.673 (0.95), | |
| | 4.333 (1.39), 4.356 (1.35), 4.785 (4.77), 6.914 (1.41), 6.935 (2.63), | |
| | 6.956 (1.26), 7.172 (2.78), 7.247 (1.88), 7.267 (1.64), 7.303 (2.21), | |
| | 7.370 (2.23), 7.390 (2.03), 7.661 (16.00), 8.732 (1.08), 12.088 | |
| | (2.03), 12.113 (1.59). | |
| 195 | 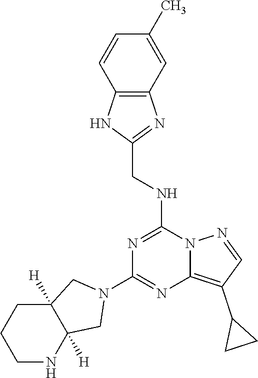 [SMILES: Cc1ccc2[nH]c(CNc3nc(N4C[C@@H]5CCCN[C@@H]5C4)nc4c(C5CC5)cnn34)nc2c1] | Intermediate 146 and CAS- RN: [151-40-0] 17% LC-MS (Method 2): Rₜ = 1.12 min; MS (ESIpos): m/z = 444 [M + H]⁺ |
| | 8-cyclopropyl-N-[(5-methyl-1H-benzimidazol-2-yl)methyl]-2- | |
| | [(4aS,7aS)-octahydro-6H-pyrrolo[3,4-b]pyridin-6-yl]pyrazolo[1,5- | |
| | a][1,3,5]triazin-4-amine | |
| | ¹H-NMR (400 MHz,DMSO-d6) δ [ppm]: 0.725 (5.00), 0.749 (2.50), | |
| | 0.760 (3.49), 0.777 (3.72), 1.233 (0.76), 1.301 (1.14), 1.348 (1.14), | |
| | 1.541 (1.21), 1.572 (1.74), 1.655 (0.83), 1.684 (0.83), 1.698 (1.21), | |
| | 1.718 (1.74), 1.730 (1.67), 1.739 (1.14), 1.751 (0.83), 2.084 (0.99), | |
| | 2.111 (0.99), 2.332 (1.59), 2.336 (0.68), 2.375 (16.00), 2.402 (1.21), | |
| | 2.427 (1.97), 2.452 (1.36), 2.518 (7.73), 2.523 (5.46), 2.539 (2.27), | |
| | 2.678 (0.76), 2.737 (1.90), 2.767 (1.59), 3.140 (1.21), 3.174 (1.44), | |
| | 3.292 (1.52), 3.365 (3.87), 3.378 (3.34), 3.389 (3.49), 3.416 (1.97), | |
| | 3.442 (1.21), 3.450 (1.21), 3.471 (1.21), 3.497 (0.53), 4.821 (4.32), | |
| | 4.834 (3.49), 6.922 (1.36), 6.941 (2.43), 6.961 (1.14), 7.191 (2.43), | |
| | 7.266 (1.29), 7.285 (1.14), 7.316 (1.82), 7.382 (1.67), 7.402 (1.44), | |
| | 7.635 (6.75), 8.619 (1.67), 12.079 (1.59). | |
| 196 | 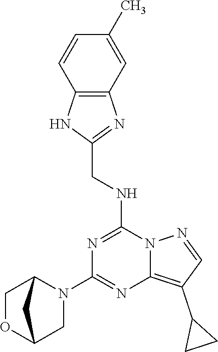 [SMILES: Cc1ccc2[nH]c(CNc3nc(N4C[C@H]5C[C@@H]4CO5)nc4c(C5CC5)cnn34)nc2c1] | Intermediate 146 and CAS- RN: [601515- 79-1] 17% LC-MS (Method 2): Rₜ = 1.03 min; MS (ESIpos): m/z = 417 [M + H]⁺ |
| | 8-cyclopropyl-N-[(5-methyl-1H-benzimidazol-2-yl)methyl]-2- | |
| | [(1R,4R)-2-oxa-5-azabicyclo[2.2.1]heptan-5-yl]pyrazolo[1,5- | |
| | a][1,3,5]triazin-4-amine | |
| | ¹H-NMR (400 MHz, DMSO-d6) δ [ppm]: 0.698 (1.26), 0.702 (1.11), | |
| | 0.710 (4.02), 0.715 (5.33), 0.722 (5.39), 0.727 (5.28), 0.735 (2.37), | |
| | 0.755 (1.17), 0.770 (3.68), 0.791 (3.52), 1.233 (1.06), 1.349 (0.52), | |
| | 1.731 (1.78), 1.752 (1.60), 1.782 (1.58), 2.368 (9.91), 2.378 (12.52), | |
| | 2.518 (5.15), 2.523 (3.34), 2.539 (1.22), 3.238 (1.40), 3.368 (1.47), | |
| | 4.540 (3.93), 4.703 (0.72), 4.798 (3.70), 4.809 (3.63), 5.759 (4.31), | |
| | 6.917 (1.44), 6.938 (2.62), 6.960 (1.35), 7.181 (2.23), 7.256 (1.29), | |
| | 7.276 (1.15), 7.309 (2.12), 7.376 (1.96), 7.397 (1.78), 7.595 (0.41), | |
| | 7.625 (0.41), 7.677 (16.00), 8.795 (1.04), 12.058 (2.14), 12.082 | |
| | (1.72). | |
| 197 | 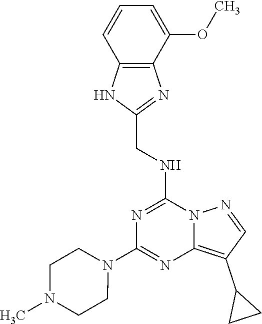 [SMILES: COc1cccc2[nH]c(CNc3nc(N4CCN(C)CC4)nc4c(C5CC5)cnn34)nc12] | Intermediate 148 and CAS- RN: [109-01-3] 28% LC-MS (Method 2): Rₜ = 1.02 min; MS (ESIpos): m/z = 434 [M + H]⁺ |
| | 8-cyclopropyl-N-[(4-methoxy-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: 0.712 (4.51), 0.718 (5.54), | |
| | 0.724 (5.23), 0.730 (4.27), 0.769 (4.42), 0.789 (4.39), 1.232 (0.58), | |
| | 1.714 (1.45), 1.720 (1.43), 1.727 (1.79), 1.733 (1.83), 1.740 (1.28), | |
| | 1.746 (1.37), 2.109 (11.04), 2.117 (13.31), 2.176 (5.73), 2.539 | |
| | (1.53), 2.728 (0.49), 2.888 (0.53), 3.245 (0.43), 3.590 (8.32), 3.884 | |
| | (16.00), 3.898 (11.78), 4.797 (5.76), 4.811 (5.69), 5.758 (0.92), | |
| | 6.621 (2.12), 6.640 (2.30), 6.720 (1.67), 6.739 (1.87), 6.974 (1.57), | |
| | 6.993 (3.06), 7.016 (1.28), 7.023 (2.33), 7.036 (2.58), 7.042 (3.00), | |
| | 7.055 (1.57), 7.062 (1.21), 7.108 (2.24), 7.128 (1.41), 7.671 (4.39), | |
| | 7.684 (5.82), 8.656 (0.66), 8.668 (1.21), 8.682 (0.66), 8.758 (0.85), | |
| | 8.773 (1.54), 12.218 (2.30), 12.553 (1.71). | |
| 198 | 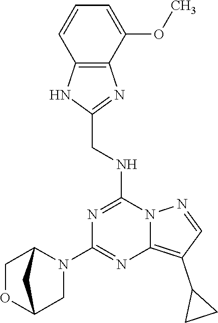 [SMILES: COc1cccc2[nH]c(CNc3nc(N4C[C@H]5C[C@@H]4CO5)nc4c(C5CC5)cnn34)nc12] | Intermediate 148 and CAS- RN: [661470- 56-0] 14% LC-MS (Method 2): Rₜ = 1.03 min; MS (ESIpos): m/z = 433 [M + H]⁺ |
| | 8-cyclopropyl-N-[(4-methoxy-1H-benzimidazol-2-yl)methyl]-2- | |
| | [(1R,4R)-2-oxa-5-azabicyclo[2.2.1]heptan-5-yl]pyrazolo[1,5- | |
| | a][1,3,5]triazin-4-amine | |
| | ¹H-NMR (400 MHz, DMSO-d6) δ [ppm]: 0.713 (6.70), 0.722 (6.71), | |
| | 0.767 (5.12), 0.787 (5.23), 1.138 (0.47), 1.233 (0.45), 1.256 (0.47), | |
| | 1.727 (2.71), 1.784 (2.56), 2.075 (2.72), 2.332 (1.83), 2.336 (0.77), | |
| | 2.518 (8.59), 2.523 (5.90), 2.539 (1.39), 2.678 (0.78), 3.247 (1.49), | |
| | 3.367 (4.85), 3.700 (0.50), 3.883 (16.00), 4.540 (6.18), 4.746 (1.12), | |
| | 4.803 (5.63), 6.620 (1.73), 6.638 (1.95), 6.718 (1.17), 6.737 (1.23), | |
| | 6.973 (1.09), 6.993 (2.08), 7.017 (3.37), 7.036 (5.16), 7.055 (2.58), | |
| | 7.106 (1.32), 7.126 (0.90), 7.677 (6.08), 8.175 (6.01), 8.716 (0.56), | |
| | 8.808 (0.83), 12.206 (2.12), 12.552 (1.20). | |
| 199 | 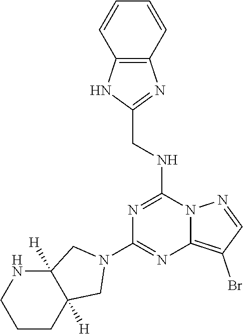 [SMILES: Brc1cnn2c(NCc3nc4ccccc4[nH]3)nc(N3C[C@H]4CCCN[C@H]4C3)nc12] | Intermediate 9 and CAS- RN: [151213- 42-2] 100% LC-MS (Method 2): Rₜ = 1.04 min; MS (ESIpos): m/z = 468 [M + H]⁺ Specific Optical Rotation (Method O1): +0.62° (c = 10 mg/mL, DMSO) |
| | N-[(1H-benzimidazol-2-yl)methyl]-8-bromo-2-[(4aR, 7aR)- | |
| | octahydro-6H-pyrrolo[3,4-b]pyridin-6-yl]pyrazolo[1,5- | |
| | a][1,3,5]triazin-4-amine | |
| | ¹H-NMR (400 MHz, DMSO-d6) δ [ppm]: 0.945 (0.63), 0.962 (0.57), | |
| | 1.169 (0.48), 1.235 (1.53), 1.307 (1.92), 1.355 (2.38), 1.488 (1.43), | |
| | 1.521 (1.80), 1.548 (2.63), 1.573 (3.01), 1.667 (1.59), 1.695 (0.98), | |
| | 1.797 (0.43), 1.904 (0.56), 2.136 (1.71), 2.291 (1.91), 2.403 (1.96), | |
| | 2.431 (3.61), 2.458 (2.94), 2.521 (12.43), 2.526 (8.02), 2.542 (0.81), | |
| | 2.741 (2.87), 2.769 (2.59), 3.152 (2.48), 3.187 (3.18), 3.283 (3.34), | |
| | 3.390 (7.56), 3.419 (2.90), 3.437 (4.74), 3.448 (3.59), 3.472 (2.95), | |
| | 3.484 (2.56), 3.504 (2.60), 3.531 (1.11), 4.829 (0.64), 4.884 (9.32), | |
| | 4.922 (0.73), 7.101 (1.14), 7.115 (4.71), 7.124 (6.39), 7.131 (6.19), | |
| | 7.137 (7.20), 7.147 (5.12), 7.160 (1.31), 7.400 (3.56), 7.418 (3.30), | |
| | 7.531 (3.63), 7.548 (3.29), 7.974 (16.00), 7.981 (13.63), 8.961 | |
| | (1.60), 12.249 (2.00), 12.270 (2.35). | |
| 200 | 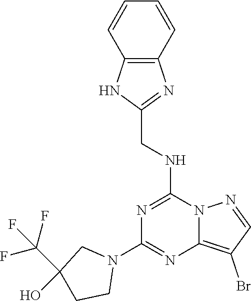 [SMILES: OC1(C(F)(F)F)CCN(c2nc(NCc3nc4ccccc4[nH]3)n3ncc(Br)c3n2)C1] | Intermediate 9 and CAS- RN: [1334147- 81-7] 72% LC-MS (Method 2): Rₜ = 1.08 min; MS (ESIpos): m/z = 497 [M + H]⁺ |
| | 1-[4-(1H-benzimidazol-2-ylmethylamino)-8-bromo-pyrazolo[1,5- | |
| | a][1,3,5]triazin-2-yl]-3-(trifluoromethyl)pyrrolidin-3-ol | |
| | ¹H-NMR (400 MHz, DMSO-d6) δ [ppm]: −0.008 (1.31), 0.008 (1.25), | |
| | 0.942 (0.64), 0.958 (0.58), 1.106 (2.76), 1.905 (0.61), 2.008 (0.81), | |
| | 2.139 (0.93), 2.161 (1.13), 2.334 (1.22), 2.520 (7.13), 2.524 (4.33), | |
| | 2.542 (0.67), 2.676 (1.19), 3.076 (0.87), 3.381 (0.41), 3.468 (0.64), | |
| | 3.487 (0.67), 3.573 (1.86), 3.588 (1.66), 3.625 (0.99), 3.658 (1.31), | |
| | 3.700 (1.63), 3.723 (1.13), 3.803 (0.70), 4.897 (3.69), 4.907 (3.43), | |
| | 6.460 (3.87), 7.101 (0.84), 7.115 (2.97), 7.120 (2.68), 7.126 (3.84), | |
| | 7.132 (4.57), 7.138 (4.04), 7.144 (2.91), 7.149 (3.40), 7.163 (0.99), | |
| | 7.399 (2.04), 7.415 (1.92), 7.528 (2.30), 7.546 (2.12), 8.039 (16.00), | |
| | 9.124 (1.08), 12.266 (3.05). | |
| 201 | 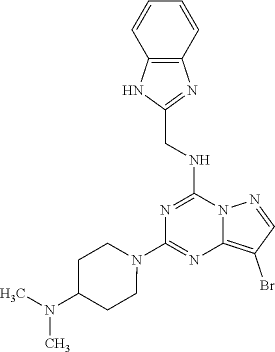 [SMILES: CN(C)C1CCN(c2nc(NCc3nc4ccccc4[nH]3)n3ncc(Br)c3n2)CC1] | Intermediate 9 and CAS- RN: [50533- 97-6] 96% LC-MS (Method 2): Rₜ = 1.06 min; MS (ESIpos): m/z = 470 [M + H]⁺ |
| | N-[(1H-benzimidazol-2-yl)methyl]-8-bromo-2-[4- | |
| | (dimethylamino)piperidin-1-yl]pyrazolo[1,5-a][1,3,5]triazin-4- | |
| | amine | |
| | ¹H-NMR (400 MHz, DMSO-d6) δ [ppm]: 1.106 (0.52), 1.156 (0.57), | |
| | 1.174 (1.05), 1.192 (0.56), 1.990 (1.88), 2.069 (16.00), 2.187 (0.52), | |
| | 2.256 (0.59), 2.724 (0.57), 2.754 (0.98), 2.782 (0.58), 4.020 (0.42), | |
| | 4.037 (0.43), 4.501 (0.52), 4.836 (2.14), 7.116 (1.42), 7.122 (1.68), | |
| | 7.128 (1.54), 7.386 (1.00), 7.403 (0.90), 7.524 (0.96), 7.541 (0.90), | |
| | 8.008 (4.69), 9.077 (0.69), 12.278 (1.23). | |
| 202 | 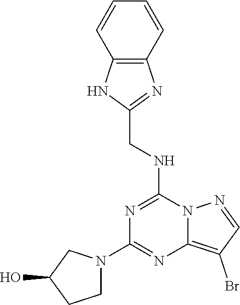 [SMILES: O[C@@H]1CCN(c2nc(NCc3nc4ccccc4[nH]3)n3ncc(Br)c3n2)C1] | Intermediate 9 and CAS- RN: [2799-21- 5] 47% LC-MS (Method 2): Rₜ = 0.90 min; MS (ESIpos): m/z = 429 [M + H]⁺ Specific Optical Rotation (Method O1): −10.28° (c = 10 mg/mL, DMSO) |
| | (3R)-1-(4-{[(1H-benzimidazol-2-yl)methyl]amino}-8- | |
| | bromopyrazolo[1,5-a][1,3,5]triazin-2-yl)pyrrolidin-3-ol | |
| | ¹H-NMR (400 MHz, DMSO-d6) δ [ppm]: 1.780 (0.57), 1.831 (0.70), | |
| | 1.863 (0.63), 1.885 (0.65), 1.908 (1.07), 2.334 (0.74), 2.520 (3.85), | |
| | 2.525 (2.40), 2.542 (0.65), 2.676 (0.76), 3.398 (2.12), 3.410 (2.39), | |
| | 3.441 (2.09), 3.462 (1.74), 3.474 (2.13), 3.503 (1.35), 3.522 (0.66), | |
| | 3.550 (0.69), 3.571 (0.74), 3.593 (0.40), 4.250 (0.86), 4.302 (0.89), | |
| | 4.879 (4.95), 4.893 (5.65), 7.124 (3.09), 7.136 (3.25), 7.413 (0.98), | |
| | 7.534 (1.02), 7.909 (1.24), 7.993 (16.00), 8.218 (0.63), 8.994 (1.43), | |
| | 9.003 (1.40), 12.256 (1.11). | |
| 203 | 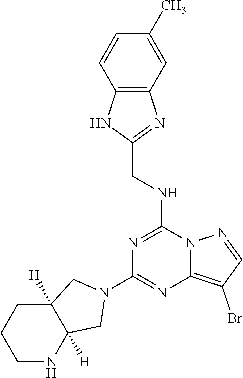 [SMILES: Cc1ccc2[nH]c(CNc3nc(N4C[C@@H]5CCCN[C@@H]5C4)nc4c(Br)cnn34)nc2c1] | Intermediate 113 and CAS- RN: [151213- 40-0] 9% LC-MS (Method 2): Rₜ = 1.11 min; MS (ESIpos): m/z = 482 [M + H]⁺ |
| | 8-bromo-N-[(5-methyl-1H-benzimidazol-2-yl)methyl]-2- | |
| | [(4aS, 7aS)-octahydro-6H-pyrrolo[3,4-b]pyridin-6-yl]pyrazolo[1,5- | |
| | a][1,3,5]triazin-4-amine | |
| | ¹H-NMR (400 MHz, DMSO-d6) δ [ppm]: 1.232 (0.63), 1.300 (1.20), | |
| | 1.348 (1.39), 1.376 (0.63), 1.486 (0.63), 1.523 (0.92), 1.536 (1.26), | |
| | 1.548 (1.29), 1.579 (1.86), 1.609 (0.76), 1.662 (0.88), 1.694 (0.50), | |
| | 1.750 (0.44), 2.131 (1.07), 2.152 (1.04), 2.331 (1.45), 2.336 (0.76), | |
| | 2.377 (16.00), 2.422 (2.11), 2.449 (1.42), 2.518 (6.28), 2.522 (4.13), | |
| | 2.539 (0.82), 2.673 (1.45), 2.678 (0.66), 2.733 (1.86), 2.762 (1.61), | |
| | 3.141 (1.42), 3.174 (1.83), 3.184 (1.07), 3.273 (0.57), 3.298 (1.67), | |
| | 3.367 (1.70), 3.381 (2.24), 3.391 (2.65), 3.418 (1.29), 3.429 (2.56), | |
| | 3.445 (1.83), 3.459 (1.33), 3.471 (1.58), 3.478 (1.58), 3.499 (1.51), | |
| | 3.526 (0.57), 4.851 (6.09), 4.889 (0.41), 5.759 (1.70), 6.927 (1.45), | |
| | 6.947 (2.56), 6.968 (1.20), 7.191 (2.52), 7.267 (1.33), 7.287 (1.20), | |
| | 7.324 (1.96), 7.389 (1.77), 7.410 (1.58), 7.965 (9.75), 7.973 (8.80), | |
| | 8.916 (0.95), 12.077 (0.82), 12.103 (1.51). | |
| 204 | 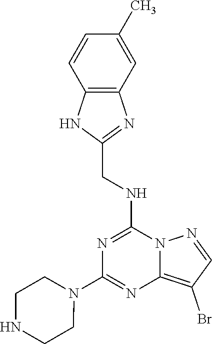 [SMILES: Cc1ccc2[nH]c(CNc3nc(N4CCNCC4)nc4c(Br)cnn34)nc2c1] | Intermediate 113 and CAS- RN: [110-85-0] 9% LC-MS (Method 2): Rₜ = 0.96 min; MS (ESIpos): m/z = 442 [M + H]⁺ |
| | 8-bromo-N-[(5-methyl-1H-benzimidazol-2-yl)methyl]-2- | |
| | (piperazin-1-yl)pyrazolo[1,5-a][1,3,5]triazin-4-amine | |
| | ¹H-NMR (400 MHz, DMSO-d6) δ [ppm]: 1.233 (0.58), 1.901 (0.46), | |
| | 2.332 (1.34), 2.336 (0.64), 2.373 (7.74), 2.382 (9.53), 2.518 (6.95), | |
| | 2.522 (4.74), 2.539 (2.73), 2.570 (2.73), 2.669 (1.97), 2.673 (1.52), | |
| | 2.678 (0.79), 3.566 (4.04), 4.825 (7.07), 6.925 (1.15), 6.947 (2.06), | |
| | 6.968 (1.00), 7.184 (2.22), 7.261 (1.49), 7.282 (1.31), 7.321 (1.73), | |
| | 7.387 (1.79), 7.407 (1.61), 7.999 (16.00), 12.083 (1.43), 12.109 | |
| | (1.12). | |
| 205 | 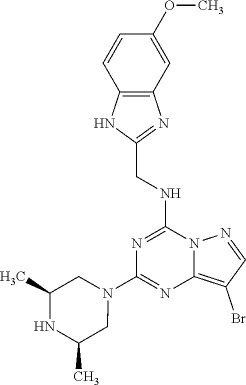 [SMILES: COc1ccc2[nH]c(CNc3nc(N4C[C@@H](C)N[C@@H](C)C4)nc4c(Br)cnn34)nc2c1] | Intermediate 150 and CAS- RN: [21655- 48-1] 17% LC-MS (Method 2): Rₜ = 1.00 min; MS (ESIpos): m/z = 486 [M + H]⁺ |
| | 8-bromo-2-[(3R,5S)-3,5-dimethylpiperazin-1-yl]-N-[(5-methoxy- | |
| | 1 H-benzimidazol-2-yl)methyl]pyrazolo[1,5-a][1,3,5]triazin-4- | |
| | amine | |
| | ¹H-NMR (400 MHz, DMSO-d6) δ [ppm]: 0.852 (1.65), 0.936 (1.61), | |
| | 1.035 (2.65), 1.053 (4.40), 1.070 (2.51), 1.751 (0.50), 2.189 (1.04), | |
| | 2.327 (2.29), 2.332 (1.75), 2.336 (0.93), 2.380 (0.43), 2.518 (7.66), | |
| | 2.523 (5.05), 2.539 (1.36), 2.669 (2.15), 2.673 (1.57), 2.678 (0.72), | |
| | 3.422 (0.64), 3.435 (0.68), 3.440 (0.64), 3.452 (0.64), 3.739 (11.20), | |
| | 3.744 (15.93), 4.343 (0.79), 4.355 (1.07), 4.368 (0.72), 4.432 (0.47), | |
| | 4.799 (3.29), 5.759 (1.50), 6.724 (1.22), 6.731 (1.36), 6.746 (1.68), | |
| | 6.752 (2.00), 6.770 (0.93), 6.777 (0.86), 6.877 (2.33), 6.883 (2.15), | |
| | 7.058 (1.54), 7.064 (1.47), 7.250 (1.50), 7.271 (1.36), 7.386 (2.11), | |
| | 7.407 (1.93), 7.996 (16.00), 9.013 (0.79), 12.075 (1.65), 12.115 | |
| | (1.22). | |
| 206 | 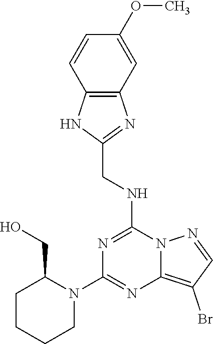 [SMILES: COc1ccc2[nH]c(CNc3nc(N4CCCC[C@H]4CO)nc4c(Br)cnn34)nc2c1] | Intermediate 150 and CAS- RN: [41373- 39-1] 7% LC-MS (Method 2): Rₜ = 1.12 min; MS (ESIpos): m/z = 487 [M + H]⁺ |
| | [(2S)-1-(8-bromo-4-{[(4-methoxy-1H-benzimidazol-2- | |
| | yl)methyl]amino}pyrazolo[1,5-a][1,3,5]triazin-2-yl)piperidin-2- | |
| | yl]methanol | |
| | ¹H-NMR (400 MHz, DMSO-d6) δ [ppm]: 1.233 (0.72), 1.349 (0.47), | |
| | 1.391 (0.56), 1.509 (1.07), 1.540 (0.80), 1.575 (0.52), 1.776 (0.54), | |
| | 1.805 (0.47), 2.332 (0.82), 2.518 (3.81), 2.522 (2.39), 2.539 (0.80), | |
| | 2.669 (1.15), 2.673 (0.84), 3.546 (0.80), 3.746 (16.00), 4.806 (0.84), | |
| | 4.856 (1.05), 4.877 (1.09), 5.758 (6.09), 6.734 (0.87), 6.740 (0.97), | |
| | 6.756 (1.07), 6.762 (1.56), 6.783 (0.64), 6.789 (0.62), 6.913 (1.09), | |
| | 7.068 (1.13), 7.073 (1.11), 7.280 (0.74), 7.301 (0.68), 7.401 (1.52), | |
| | 7.423 (1.40), 7.979 (6.57), 8.971 (0.70). | |
| 207 | 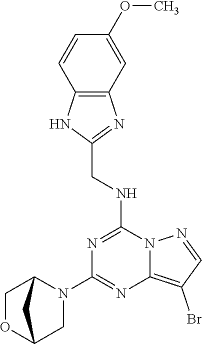 [SMILES: COc1ccc2[nH]c(CNc3nc(N4C[C@H]5C[C@@H]4CO5)nc4c(Br)cnn34)nc2c1] | Intermediate 150 and CAS- RN: [661470- 56-0] 12% LC-MS (Method 2): Rₜ = 0.96 min; MS (ESIpos): m/z = 471 [M + H]⁺ |
| | 8-bromo-N-[(4-methoxy-1H-benzimidazol-2-yl)methyl]-2- | |
| | [(1R,4R)-2-oxa-5-azabicyclo[2.2.1]heptan-5-yl]pyrazolo[1,5- | |
| | a][1,3,5]triazin-4-amine | |
| | ¹H-NMR (400 MHz,DMSO-d6) δ [ppm]: 1.232 (0.66), 1.729 (0.87), | |
| | 1.751 (1.17), 1.787 (1.24), 1.813 (1.49), 2.318 (0.66), 2.460 (0.98), | |
| | 2.465 (1.28), 2.518 (7.00), 2.523 (4.52), 2.533 (0.44), 2.539 (3.06), | |
| | 2.678 (0.66), 3.244 (0.44), 3.295 (1.64), 3.384 (2.04), 3.411 (0.69), | |
| | 3.694 (0.58), 3.742 (13.81), 3.747 (15.31), 4.568 (2.77), 4.737 | |
| | (0.77), 4.761 (1.09), 4.818 (3.46), 4.889 (0.77), 5.758 (9.66), 6.727 | |
| | (1.13), 6.733 (1.28), 6.755 (2.08), 6.774 (0.87), 6.884 (1.20), 7.066 | |
| | (1.42), 7.256 (0.66), 7.276 (0.73), 7.392 (1.49), 7.414 (1.38), 8.012 | |
| | (16.00), 9.075 (0.69), 12.037 (2.00), 12.082 (1.38). | |
| 208 | 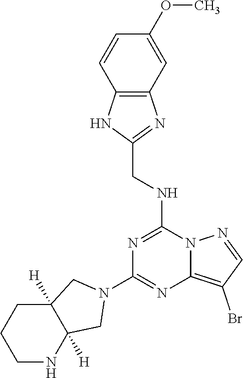 [SMILES: COc1ccc2[nH]c(CNc3nc(N4C[C@@H]5CCCN[C@@H]5C4)nc4c(Br)cnn34)nc2c1] | Intermediate 150 and CAS- RN: [151213- 40-0] 7% LC-MS (Method 2): Rₜ = 1.04 min; MS (ESIpos): m/z = 498 [M + H]⁺ |
| | 8-bromo-N-[(4-methoxy-1H-benzimidazol-2-yl)methyl]-2- | |
| | [(4aS,7aS)-octahydro-6H-pyrrolo[3,4-b]pyridin-6-yl]pyrazolo[1,5- | |
| | a][1,3,5]triazin-4-amine | |
| | ¹H-NMR (400 MHz, DMSO-d6) δ [ppm]: 1.233 (0.77), 1.302 (0.52), | |
| | 1.349 (0.79), 1.544 (0.58), 1.584 (0.82), 2.129 (0.60), 2.402 (0.47), | |
| | 2.427 (0.92), 2.459 (0.66), 2.518 (3.10), 2.523 (2.02), 2.539 (0.76), | |
| | 2.673 (0.69), 2.738 (0.84), 2.769 (0.69), 3.150 (0.57), 3.180 (0.81), | |
| | 3.387 (0.95), 3.396 (0.93), 3.434 (1.04), 3.450 (0.73), 3.464 (0.52), | |
| | 3.476 (0.62), 3.504 (0.52), 3.745 (16.00), 4.836 (2.13), 6.729 (0.57), | |
| | 6.735 (0.66), 6.756 (1.07), 6.776 (0.46), 6.901 (1.07), 7.072 (0.76), | |
| | 7.268 (0.58), 7.290 (0.52), 7.397 (0.95), 7.419 (0.88), 7.965 (4.74), | |
| | 7.972 (3.93), 8.913 (0.47), 12.061 (0.49). | |
| 209 | 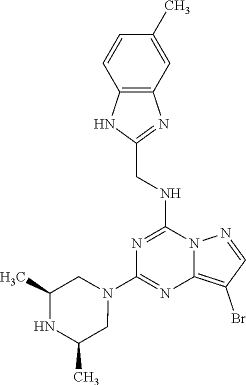 [SMILES: Cc1ccc2[nH]c(CNc3nc(N4C[C@@H](C)N[C@@H](C)C4)nc4c(Br)cnn34)nc2c1] | Intermediate 113 and CAS- RN: [111-21- 91] 17% LC-MS (Method 2): Rₜ = 1.07 min; MS (ESIneg): m/z = 468 [M − H]⁻ |
| | 8-bromo-2-[(3R,5S)-3,5-dimethylpiperazin-1-yl]-N-[(5-methyl-1H- | |
| | benzimidazol-2-yl)methyl]pyrazolo[1,5-a][1,3,5]triazin-4-amine | |
| | ¹H-NMR (400 MHz, DMSO-d6) δ [ppm]: 0.828 (2.02), 0.916 (2.30), | |
| | 0.931 (2.40), 1.149 (0.67), 1.166 (0.66), 2.188 (1.11), 2.290 (0.50), | |
| | 2.322 (1.44), 2.326 (1.81), 2.332 (1.46), 2.369 (10.52), 2.378 | |
| | (13.04), 2.518 (5.34), 2.522 (3.78), 2.539 (4.30), 2.554 (1.27), 2.664 | |
| | (1.07), 2.668 (1.40), 2.673 (1.01), 4.315 (0.58), 4.437 (0.59), 4.811 | |
| | (5.07), 5.756 (9.93), 6.920 (1.42), 6.942 (2.64), 6.963 (1.36), 7.174 | |
| | (2.27), 7.250 (1.20), 7.270 (1.08), 7.312 (1.80), 7.379 (1.58), 7.399 | |
| | (1.43), 7.996 (16.00), 9.017 (0.59), 12.107 (1.66), 12.133 (1.32). | |
| 210 | 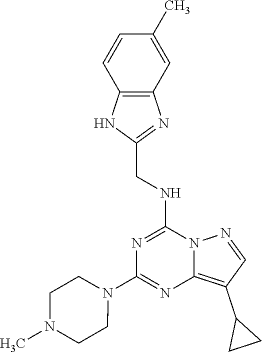 [SMILES: Cc1ccc2[nH]c(CNc3nc(N4CCN(C)CC4)nc4c(C5CC5)cnn34)nc2c1] | Intermediate 146 and CAS- RN: [109-01-3] 18% LC-MS (Method 2): Rₜ = 1.07 min; MS (ESIpos): m/z = 418 [M + H]⁺ |
| | 8-cyclopropyl-N-[(5-methyl-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: 0.700 (1.04), 0.704 (0.68), | |
| | 0.712 (3.06), 0.717 (3.74), 0.724 (4.60), 0.729 (3.83), 0.738 (1.89), | |
| | 0.750 (0.59), 0.757 (0.77), 0.764 (1.94), 0.771 (3.74), 0.776 (2.48), | |
| | 0.784 (2.52), 0.788 (1.62), 0.792 (3.74), 0.797 (2.39), 0.803 (0.77), | |
| | 0.809 (1.13), 1.232 (0.63), 1.702 (0.59), 1.714 (1.13), 1.722 (1.17), | |
| | 1.735 (1.89), 1.743 (0.72), 1.748 (1.08), 1.756 (1.04), 1.768 (0.50), | |
| | 2.084 (0.45), 2.119 (16.00), 2.186 (3.79), 2.332 (0.68), 2.377 (7.89), | |
| | 2.518 (2.97), 2.523 (2.12), 2.539 (0.63), 2.673 (0.63), 3.589 (4.24), | |
| | 4.803 (2.88), 4.815 (2.84), 6.922 (0.72), 6.942 (1.26), 6.963 (0.59), | |
| | 7.183 (1.26), 7.259 (0.63), 7.279 (0.59), 7.312 (0.95), 7.378 (0.86), | |
| | 7.399 (0.77), 7.682 (10.64), 8.760 (0.99), 12.076 (0.86), 12.095 | |
| | (0.68). | |
| 211 | 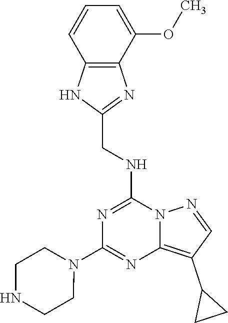 [SMILES: COc1cccc2[nH]c(CNc3nc(N4CCNCC4)nc4c(C5CC5)cnn34)nc12] | Intermediate 148 and CAS- RN: [110-85-0] 15% LC-MS (Method 2): Rₜ = 0.95 min; MS (ESIpos): m/z = 420 [M + H]⁺ |
| | 8-cyclopropyl-N-[(4-methoxy-1H-benzimidazol-2-yl)methyl]-2- | |
| | (piperazin-1-yl)pyrazolo[1,5-a][1,3,5]triazin-4-amine | |
| | ¹H-NMR (400 MHz, DMSO-d6) δ [ppm]: 0.701 (1.28), 0.714 (4.91), | |
| | 0.719 (4.94), 0.726 (5.98), 0.757 (2.88), 0.764 (4.97), 0.769 (3.75), | |
| | 0.777 (3.12), 0.785 (5.25), 0.802 (1.23), 1.232 (1.01), 1.695 (0.59), | |
| | 1.708 (1.24), 1.715 (1.30), 1.725 (2.00), 1.737 (1.28), 1.751 (1.21), | |
| | 1.761 (0.47), 2.318 (0.70), 2.518 (5.33), 2.523 (3.83), 2.539 (3.49), | |
| | 2.573 (6.79), 2.678 (0.71), 3.520 (6.81), 3.532 (8.81), 3.544 (6.47), | |
| | 3.885 (16.00), 3.899 (10.13), 4.798 (9.60), 5.759 (3.73), 6.621 | |
| | (1.91), 6.640 (2.14), 6.720 (1.42), 6.740 (1.56), 6.975 (1.41), 6.994 | |
| | (2.84), 7.020 (2.55), 7.039 (3.54), 7.056 (1.58), 7.109 (1.91), 7.129 | |
| | (1.20), 7.662 (3.77), 7.674 (5.86), 12.214 (1.59), 12.552 (0.59). | |
| 212 | 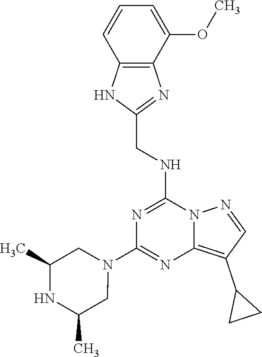 [SMILES: COc1cccc2[nH]c(CNc3nc(N4C[C@@H](C)N[C@@H](C)C4)nc4c(C5CC5)cnn34)nc12] | Intermediate 148 and CAS- RN: [111-21- 91] 11% LC-MS (Method 2): Rₜ = 1.06 min; MS (ESIpos): m/z = 448 [M + H]⁺ |
| | 8-cyclopropyl-2-[(3R,5S)-3,5-dimethylpiperazin-1-yl]-N-[(4- | |
| | methoxy-1H-benzimidazol-2-yl)methyl]pyrazolo[1,5- | |
| | a][1,3,5]triazin-4-amine | |
| | ¹H-NMR (400 MHz, DMSO-d6) δ [ppm]: 0.689 (1.41), 0.702 (4.64), | |
| | 0.707 (5.61), 0.714 (6.00), 0.719 (5.32), 0.727 (3.05), 0.748 (1.36), | |
| | 0.762 (3.44), 0.769 (6.04), 0.775 (4.47), 0.783 (4.07), 0.787 (3.13), | |
| | 0.791 (6.71), 0.796 (4.62), 0.807 (2.21), 0.880 (7.25), 0.938 (1.11), | |
| | 0.948 (1.06), 0.977 (0.58), 1.216 (0.50), 1.225 (1.28), 1.232 (1.51), | |
| | 1.246 (1.26), 1.254 (1.12), 1.266 (1.02), 1.275 (1.04), 1.294 (0.55), | |
| | 1.304 (0.44), 1.349 (0.52), 1.478 (1.49), 1.508 (1.10), 1.681 (0.59), | |
| | 1.699 (1.35), 1.712 (2.05), 1.720 (1.92), 1.732 (2.99), 1.746 (1.70), | |
| | 1.749 (1.59), 1.753 (1.71), 1.767 (0.71), 2.102 (1.59), 2.130 (2.68), | |
| | 2.160 (1.84), 2.287 (0.51), 2.318 (0.81), 2.453 (2.79), 2.518 (6.34), | |
| | 2.523 (4.38), 2.539 (1.08), 2.920 (0.84), 2.953 (0.66), 3.175 (0.68), | |
| | 3.262 (0.86), 3.879 (16.00), 4.327 (1.66), 4.532 (0.62), 4.780 | |
| | (11.13), 5.759 (0.45), 6.613 (1.17), 6.632 (1.32), 6.710 (0.82), 6.730 | |
| | (0.85), 6.960 (0.89), 6.980 (1.60), 7.011 (2.82), 7.031 (4.53), 7.050 | |
| | (2.29), 7.097 (1.02), 7.116 (0.66), 7.658 (4.86), 12.227 (0.82), | |
| | 12.546 (0.40). | |
| 213 | 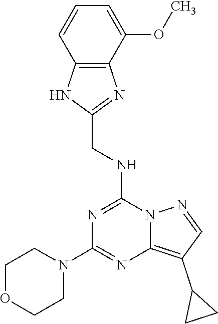 [SMILES: COc1cccc2[nH]c(CNc3nc(N4CCOCC4)nc4c(C5CC5)cnn34)nc12] | Intermediate 148 and CAS- RN: [110-91-8] 24% LC-MS (Method 2): Rₜ = 1.10 min; MS (ESIpos): m/z = 421 [M + H]⁺ |
| | 8-cyclopropyl-N-[(4-methoxy-1H-benzimidazol-2-yl)methyl]-2- | |
| | morpholino-pyrazolo[1,5-a][1,3,5]triazin-4-amine | |
| | ¹H-NMR (400 MHz, DMSO-d6) δ [ppm]: 0.709 (1.01), 0.721 (3.90), | |
| | 0.727 (4.61), 0.734 (4.87), 0.739 (3.26), 0.747 (1.48), 0.764 (2.16), | |
| | 0.772 (3.77), 0.786 (2.49), 0.793 (3.85), 0.807 (0.91), 1.233 (0.78), | |
| | 1.706 (0.49), 1.719 (1.10), 1.727 (1.10), 1.734 (1.51), 1.739 (1.46), | |
| | 1.748 (1.02), 1.753 (1.14), 1.760 (0.76), 2.518 (2.94), 2.523 (2.05), | |
| | 3.501 (6.59), 3.571 (7.28), 3.582 (8.01), 3.884 (16.00), 3.899 (10.56), | |
| | 4.807 (4.45), 4.820 (4.45), 6.625 (1.85), 6.642 (2.01), 6.724 (1.46), | |
| | 6.743 (1.59), 6.977 (1.41), 6.995 (2.73), 7.018 (1.14), 7.025 (2.40), | |
| | 7.038 (2.29), 7.045 (2.92), 7.057 (1.38), 7.064 (1.15), 7.110 (1.92), | |
| | 7.130 (1.20), 7.691 (4.21), 7.705 (6.08), 8.719 (0.91), 8.815 (0.63), | |
| | 8.829 (1.22), 12.218 (1.75), 12.562 (1.28). | |
| 214 | 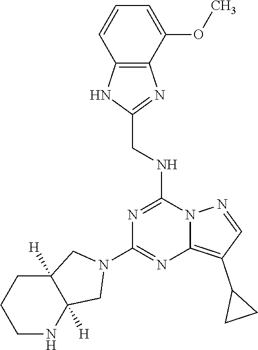 [SMILES: COc1cccc2[nH]c(CNc3nc(N4C[C@@H]5CCCN[C@@H]5C4)nc4c(C5CC5)cnn34)nc12] | Intermediate 148 and CAS- RN: [151213- 40-0] 24% LC-MS (Method 2): Rₜ = 1.10 min; MS (ESIpos): m/z = 460 [M + H]⁺ |
| | 8-cyclopropyl-N-[(4-methoxy-1H-benzimidazol-2-yl)methyl]-2- | |
| | [(4aS,7aS)-octahydro-6H-pyrrolo[3,4-b]pyridin-6-yl]pyrazolo[1,5- | |
| | a][1,3,5]triazin-4-amine | |
| | ¹H-NMR (400 MHz, DMSO-d6) δ [ppm]: 0.726 (4.96), 0.776 (3.65), | |
| | 1.233 (1.04), 1.314 (1.44), 1.349 (1.54), 1.547 (1.34), 1.587 (1.88), | |
| | 1.658 (0.90), 1.713 (1.65), 1.729 (1.62), 1.752 (1.83), 2.119 (1.00), | |
| | 2.408 (1.14), 2.435 (2.14), 2.459 (1.55), 2.518 (4.99), 2.523 (3.39), | |
| | 2.539 (1.49), 2.742 (1.91), 2.771 (1.54), 3.176 (1.63), 3.283 (1.06), | |
| | 3.382 (2.85), 3.417 (2.07), 3.445 (1.29), 3.473 (0.97), 3.886 (16.00), | |
| | 3.896 (10.52), 4.825 (3.50), 5.759 (9.57), 6.622 (1.86), 6.640 (2.08), | |
| | 6.720 (1.34), 6.739 (1.41), 6.982 (1.26), 7, 000 (2.77), 7.021 (2.98), | |
| | 7.040 (4.13), 7.060 (1.93), 7.112 (1.30), 7.132 (0.83), 7.637 (4.13), | |
| | 8.537 (0.43), 8.631 (0.83), 12.214 (1.11), 12.549 (0.84). | |
| 215 | 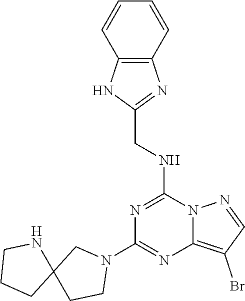 [SMILES: Brc1cnn2c(NCc3nc4ccccc4[nH]3)nc(N3CCC4(CCCN4)C3)nc12] | Intermediate 9 and CAS- RN: [82386- 80-9] 54% LC-MS (Method 2): Rₜ = 1.02 min; MS (ESIpos): m/z = 468 [M + H]⁺ |
| | N-(1H-benzimidazol-2-ylmethyl)-8-bromo-2-(1,7- | |
| | diazaspiro[4.4]nonan-7-yl)pyrazolo[1,5-a][1,3,5]triazin-4-amine | |
| | ¹H-NMR (400 MHz, DMSO-d6) δ [ppm]: 1.547 (1.48), 1.563 (2.11), | |
| | 1.605 (1.18), 1.623 (1.25), 1.657 (0.87), 1.715 (1.86), 1.784 (1.84), | |
| | 1.803 (1.87), 1.813 (1.78), 1.834 (1.03), 2.322 (0.92), 2.327 (1.28), | |
| | 2.332 (0.93), 2.518 (5.08), 2.523 (3.20), 2.637 (0.53), 2.660 (0.88), | |
| | 2.664 (1.22), 2.669 (1.57), 2.673 (1.12), 2.751 (0.40), 2.775 (0.60), | |
| | 2.787 (0.60), 2.822 (1.22), 2.838 (1.92), 2.853 (1.09), 3.176 (0.84), | |
| | 3.205 (1.63), 3.249 (1.92), 3.278 (1.83), 3.417 (2.33), 3.445 (2.74), | |
| | 3.463 (1.48), 3.489 (1.43), 3.506 (2.12), 3.523 (1.19), 4.856 (3.56), | |
| | 4.884 (4.02), 7.121 (3.17), 7.134 (3.47), 7.407 (1.07), 7.524 (1.11), | |
| | 7.986 (16.00), 12.257 (0.96). | |
| 216 | 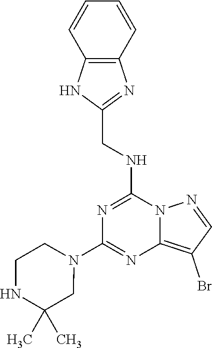 [SMILES: CC1(C)CN(c2nc(NCc3nc4ccccc4[nH]3)n3ncc(Br)c3n2)CCN1] | Intermediate 9 and CAS- RN: [84477- 72-5] 50% LC-MS (Method 2): Rₜ = 0.97 min; MS (ESIpos): m/z = 456 [M + H]⁺ |
| | N-[(1H-benzimidazol-2-yl)methyl]-8-bromo-2-(3,3- | |
| | dimethylpiperazin-1-yl)pyrazolo[1,5-a][1,3,5]triazin-4-amine | |
| | ¹H-NMR (400 MHz, DMSO-d6) δ [ppm]: 0.623 (1.90), 0.959 (0.64), | |
| | 1.234 (0.44), 2.086 (16.00), 2.325 (0.85), 2.329 (1.16), 2.334 (0.85), | |
| | 2.520 (4.82), 2.525 (3.16), 2.542 (5.51), 2.632 (1.11), 2.662 (1.12), | |
| | 2.667 (1.46), 2.671 (1.66), 2.676 (1.27), 3.560 (1.34), 4.815 (2.40), | |
| | 7.105 (2.25), 7.112 (3.05), 7.120 (2.98), 7.127 (3.37), 7.135 (2.33), | |
| | 7.148 (0.60), 7.372 (1.42), 7.389 (1.32), 7.516 (1.60), 7.533 (1.44), | |
| | 7.997 (14.58), 8.012 (0.55), 12.267 (2.29). | |
| 217 | 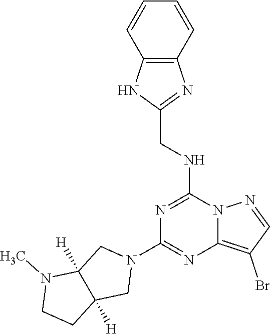 [SMILES: CN1CC[C@@H]2CN(c3nc(NCc4nc5ccccc5[nH]4)n4ncc(Br)c4n3)C[C@@H]21] | Intermediate 9 and CAS- RN: [1353644- 77-5] 40% LC-MS (Method 2): Rₜ = 1.11 min; MS (ESIpos): m/z = 468 [M + H]⁺ |
| | N-[(1H-benzimidazol-2-yl)methyl]-8-bromo-2-[(3aR,6aR)-1- | |
| | methylhexahydropyrrolo[3,4-b]pyrrol-5 (1H)-yl]pyrazolo[1,5- | |
| | a][1,3,5]triazin-4-amine | |
| | ¹H-NMR (400 MHz, DMSO-d6) δ [ppm]: 1.234 (0.68), 1.258 (0.44), | |
| | 1.373 (0.53), 1.565 (0.48), 1.909 (0.64), 2.003 (2.15), 2.086 (1.08), | |
| | 2.153 (1.32), 2.175 (1.39), 2.216 (2.12), 2.320 (0.54), 2.324 (1.04), | |
| | 2.329 (1.43), 2.334 (1.06), 2.338 (0.49), 2.520 (4.67), 2.525 (3.15), | |
| | 2.542 (2.50), 2.662 (1.13), 2.667 (1.65), 2.671 (2.02), 2.676 (1.57), | |
| | 2.680 (1.03), 2.743 (1.05), 2.901 (1.20), 2.921 (1.93), 2.941 (1.18), | |
| | 3.168 (2.92), 3.491 (0.81), 3.626 (0.99), 3.656 (0.92), 4.854 (3.99), | |
| | 4.868 (3.91), 7.118 (2.98), 7.131 (3.14), 7.396 (1.14), 7.413 (1.21), | |
| | 7.432 (0.60), 7.449 (0.56), 7.524 (1.08), 7.807 (0.41), 7.990 (16.00), | |
| | 8.150 (0.44), 9.020 (1.56), 12.256 (0.91). | |
| 218 | 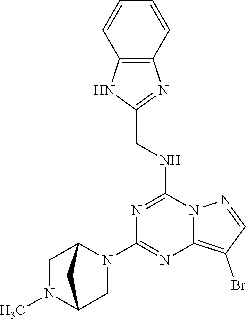 [SMILES: CN1C[C@H]2C[C@@H]1CN2c1nc(NCc2nc3ccccc3[nH]2)n2ncc(Br)c2n1] | Intermediate 9 and CAS- RN: [1403763- 25-6] 38% LC-MS (Method 2): Rₜ = 0.93 min; MS (ESIpos): m/z = 454 [M + H]⁺ Specific Optical Rotation (Method O1): +45.32° (c = 10 mg/mL, DMSO) |
| | N-[(1H-benzimidazol-2-yl)methyl]-8-bromo-2-[(1R,4R)-5-methyl- | |
| | 2, 5-diazabicyclo[2.2.1]heptan-2-yl]pyrazolo[1,5-a][1,3,5]triazin-4- | |
| | amine | |
| | ¹H-NMR (400 MHz, DMSO-d6) δ [ppm]: 1.234 (1.61), 1.592 (0.63), | |
| | 1.615 (1.02), 1.652 (0.56), 1.691 (0.88), 1.714 (0.61), 1.754 (0.78), | |
| | 1.783 (0.54), 1.804 (0.40), 1.905 (2.01), 2.031 (0.85), 2.055 (0.95), | |
| | 2.076 (1.34), 2.086 (0.48), 2.141 (3.85), 2.163 (6.82), 2.334 (0.95), | |
| | 2.339 (0.48), 2.363 (0.84), 2.388 (0.80), 2.405 (0.61), 2.429 (0.72), | |
| | 2.521 (4.42), 2.525 (2.99), 2.542 (4.98), 2.676 (0.92), 2.806 (0.52), | |
| | 2.829 (0.46), 3.127 (0.53), 3.152 (0.64), 3.191 (0.53), 3.206 (0.97), | |
| | 3.230 (1.18), 3.430 (1.01), 3.523 (1.09), 3.549 (0.89), 4.497 (1.43), | |
| | 4.630 (0.87), 4.832 (1.75), 4.845 (2.27), 7.095 (0.47), 7.108 (1.86), | |
| | 7.118 (2.59), 7.124 (2.41), 7.130 (2.77), 7.141 (2.04), 7.153 (0.51), | |
| | 7.387 (1.24), 7.404 (1.18), 7.521 (1.31), 7.539 (1.16), 7.995 (16.00), | |
| | 9.030 (0.93), 9.044 (1.84), 9.058 (0.88), 12.239 (1.57). | |
| 219 | 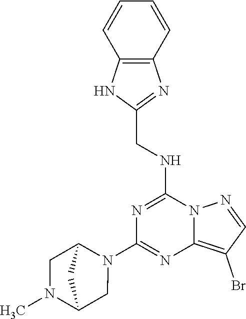 [SMILES: CN1C[C@@H]2C[C@H]1CN2c1nc(NCc2nc3ccccc3[nH]2)n2ncc(Br)c2n1] | Intermediate 9 and CAS- RN: [125224- 62-6] 65% LC-MS (Method 2): Rₜ = 0.93 min; MS (ESIpos): m/z = 454 [M + H]⁺ Specific Optical Rotation (Method O1): −43.07° (c = 10 mg/mL, DMSO) |
| | N-[(1H-benzimidazol-2-yl)methyl]-8-bromo-2-[(1S,4S)-5-methyl- | |
| | 2, 5-diazabicyclo[2.2.1]heptan-2-yl]pyrazolo[1,5-a][1,3,5]triazin-4- | |
| | amine | |
| | ¹H-NMR (400 MHz, DMSO-d6) δ [ppm]: 1.234 (0.66), 1.589 (0.53), | |
| | 1.612 (0.88), 1.649 (0.47), 1.688 (0.77), 1.711 (0.52), 1.754 (0.40), | |
| | 1.779 (0.45), 1.899 (0.94), 2.027 (0.74), 2.050 (0.81), 2.139 (3.43), | |
| | 2.161 (6.13), 2.334 (0.70), 2.360 (0.69), 2.385 (0.65), 2.400 (0.49), | |
| | 2.424 (0.52), 2.521 (3.72), 2.525 (2.57), 2.542 (4.41), 2.676 (0.72), | |
| | 2.805 (0.46), 2.828 (0.41), 3.125 (0.41), 3.152 (0.48), 3.205 (0.72), | |
| | 3.229 (0.82), 3.402 (0.79), 3.430 (0.60), 3.522 (0.90), 3.548 (0.75), | |
| | 4.496 (1.27), 4.628 (0.77), 4.845 (1.94), 7.095 (0.40), 7.108 (1.66), | |
| | 7.118 (2.28), 7.124 (2.16), 7.130 (2.44), 7.140 (1.81), 7.154 (0.44), | |
| | 7.386 (1.11), 7.404 (1.05), 7.520 (1.17), 7.539 (1.04), 7.995 (16.00), | |
| | 9.044 (1.35), 12.239 (1.44). | |
| 220 | 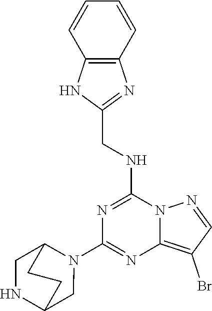 [SMILES: Brc1cnn2c(NCc3nc4ccccc4[nH]3)nc(N3CC4CCC3CN4)nc12] | Intermediate 9 and CAS- RN: [1192-92- 3] 10% LC-MS (Method 2): Rₜ = 0.93 min; MS (ESIpos): m/z = 454 [M + H]⁺ |
| | N-(1H-benzimidazol-2-ylmethyl)-8-bromo-2-(2,5- | |
| | diazabicyclo[2.2.2]octan-2-yl)pyrazolo[1,5-a][1,3,5]triazin-4- | |
| | amine | |
| | ¹H-NMR (400 MHz, DMSO-d6) δ [ppm]: 0.835 (0.98), 0.854 (1.77), | |
| | 1.236 (10.14), 1.619 (3.82), 1.705 (2.54), 1.719 (2.65), 1.743 (1.64), | |
| | 1.754 (2.87), 1.875 (3.00), 1.908 (4.61), 1.968 (2.18), 2.087 (0.55), | |
| | 2.179 (0.52), 2.302 (4.83), 2.330 (5.10), 2.334 (2.89), 2.521 (15.68), | |
| | 2.526 (9.95), 2.542 (15.93), 2.667 (2.79), 2.672 (3.72), 2.676 (2.69), | |
| | 2.944 (0.74), 2.963 (1.65), 2.993 (2.88), 3.045 (3.53), 3.076 (2.06), | |
| | 3.278 (11.99), 3.532 (2.27), 3.565 (2.88), 3.592 (3.78), 3.624 (5.79), | |
| | 3.666 (2.54), 3.698 (1.67), 3.730 (2.98), 3.761 (1.96), 4.617 (3.70), | |
| | 4.795 (2.61), 4.846 (8.67), 4.887 (1.30), 4.922 (3.97), 4.933 (3.68), | |
| | 4.974 (0.59), 7.142 (8.79), 7.411 (3.42), 7.531 (3.72), 7.990 (0.54), | |
| | 8.020 (1.41), 8.056 (16.00), 8.183 (1.09), 9.168 (0.56), 12.284 | |
| | (4.09). | |
| 221 | 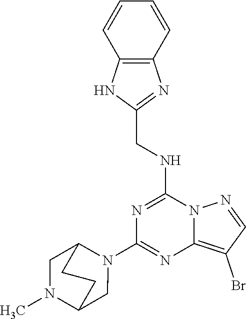 [SMILES: CN1CC2CCC1CN2c1nc(NCc2nc3ccccc3[nH]2)n2ncc(Br)c2n1] | Intermediate 9 and CAS- RN: [52321- 18-3] 48% LC-MS (Method 2): Rₜ = 1.02 min; MS (ESIpos): m/z = 468 [M + H]⁺ |
| | N-(1H-benzimidazol-2-ylmethyl)-8-bromo-2-(5-methyl-2, 5- | |
| | diazabicyclo[2.2.2]octan-2-yl)pyrazolo[1,5-a][1,3,5]triazin-4- | |
| | amine | |
| | ¹H-NMR (400 MHz, DMSO-d6) δ [ppm]: 1.234 (1.14), 1.422 (1.42), | |
| | 1.447 (4.53), 1.476 (1.41), 1.724 (1.07), 1.872 (0.94), 1.908 (1.43), | |
| | 2.227 (12.09), 2.325 (1.17), 2.329 (1.69), 2.334 (1.16), 2.459 (1.22), | |
| | 2.521 (5.82), 2.525 (4.01), 2.542 (2.58), 2.629 (1.53), 2.662 (1.70), | |
| | 2.667 (2.39), 2.671 (2.92), 2.676 (2.27), 2.730 (2.60), 2.829 (1.12), | |
| | 2.853 (0.74), 3.267 (1.66), 3.398 (2.62), 3.626 (0.95), 3.657 (0.80), | |
| | 3.716 (1.42), 3.746 (1.20), 3.891 (0.87), 4.369 (1.95), 4.566 (1.33), | |
| | 4.807 (3.34), 4.821 (3.46), 4.890 (2.43), 4.904 (2.41), 7.116 (3.20), | |
| | 7.128 (3.73), 7.138 (3.25), 7.379 (1.22), 7.401 (1.58), 7.420 (0.88), | |
| | 7.514 (1.33), 7.531 (1.85), 7.992 (16.00), 8.999 (0.89), 9.012 (1.76), | |
| | 9.027 (1.25), 9.046 (1.13), 12.228 (1.64), 12.254 (1.17). | |
| 222 | 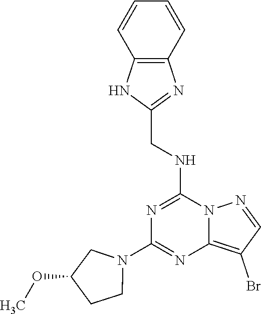 [SMILES: CO[C@H]1CCN(c2nc(NCc3nc4ccccc4[nH]3)n3ncc(Br)c3n2)C1] | Intermediate 9 and CAS- RN: [685828- 16-4] 100% LC-MS (Method 2): Rₜ = 1.05 min; MS (ESIpos): m/z = 443 [M + H]⁺ Specific Optical Rotation (Method O1): +7.83° (c = 10 mg/mL, DMSO) |
| | N-[(1H-benzimidazol-2-yl)methyl]-8-bromo-2-[(3S)-3- | |
| | methoxypyrrolidin-1-yl]pyrazolo[1,5-a][1,3,5]triazin-4-amine | |
| | ¹H-NMR (400 MHz, DMSO-d6) δ [ppm]: −0.012 (0.65), −0.003 | |
| | (16.00), 0.005 (0.71), 1.102 (7.44), 1.904 (1.33), 1.941 (1.57), 2.522 | |
| | (3.19), 3.057 (4.84), 3.072 (3.23), 3.217 (4.45), 3.252 (0.58), 3.435 | |
| | (0.49), 3.461 (1.20), 3.497 (1.52), 3.552 (1.50), 3.580 (0.70), 3.893 | |
| | (0.84), 3.986 (0.80), 4.874 (4.33), 7.092 (0.57), 7.123 (3.22), 7.154 | |
| | (0.71), 7.393 (2.12), 7.410 (1.85), 7.526 (2.15), 7.542 (2.03), 7.996 | |
| | (12.63), 9.034 (1.06), 12.254 (1.71). | |
| 223 | 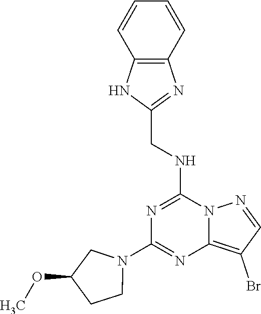 [SMILES: CO[C@@H]1CCN(c2nc(NCc3nc4ccccc4[nH]3)n3ncc(Br)c3n2)C1] | Intermediate 9 and CAS- RN: [474707- 30-7] 100% LC-MS (Method 2): Rₜ = 1.05 min; MS (ESIpos): m/z = 443 [M + H]⁺ Specific Optical Rotation (Method O1): −7.77° (c = 10 mg/mL, DMSO) |
| | N-[(1H-benzimidazol-2-yl)methyl]-8-bromo-2-[(3R)-3- | |
| | methoxypyrrolidin-1-yl]pyrazolo[1,5-a][1,3,5]triazin-4-amine | |
| | ¹H-NMR (400 MHz, DMSO-d6) δ [ppm]: 1.106 (12.05), 1.175 (0.72), | |
| | 1.907 (1.80), 1.945 (2.22), 1.990 (1.82), 2.676 (0.88), 3.061 (6.70), | |
| | 3.076 (5.03), 3.220 (6.11), 3.246 (0.80), 3.256 (0.76), 3.463 (1.64), | |
| | 3.501 (2.05), 3.555 (2.10), 3.584 (1.01), 3.896 (1.20), 3.988 (1.14), | |
| | 4.020 (0.49), 4.878 (6.08), 7.095 (0.80), 7.126 (4.41), 7.156 (0.99), | |
| | 7.396 (2.88), 7.413 (2.52), 7.529 (2.90), 7.546 (2.76), 8.000 (16.00), | |
| | 9.038 (1.49), 12.256 (2.36). | |
| 224 | 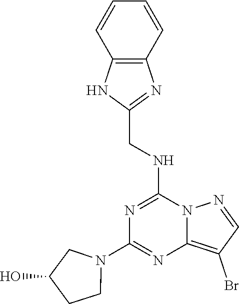 [SMILES: O[C@H]1CCN(c2nc(NCc3nc4ccccc4[nH]3)n3ncc(Br)c3n2)C1] | Intermediate 9 and CAS- RN: [122536- 94-1] 43% LC-MS (Method 2): Rₜ = 0.90 min; MS (ESIpos): m/z = 429 [M + H]⁺ Specific Optical Rotation (Method O1): +13.05° (c = 10 mg/mL, DMSO) |
| | (3S)-1-(4-{[(1H-benzimidazol-2-yl)methyl]amino}-8- | |
| | bromopyrazolo[1,5-a][1,3,5]triazin-2-yl)pyrrolidin-3-ol | |
| | ¹H-NMR (400 MHz, DMSO-d6) δ [ppm]: 1.781 (0.52), 1.830 (0.62), | |
| | 1.864 (0.54), 1.886 (0.59), 1.909 (0.92), 1.936 (0.41), 2.334 (0.70), | |
| | 2.521 (3.41), 2.526 (2.18), 2.542 (0.51), 2.676 (0.72), 3.408 (3.44), | |
| | 3.441 (2.42), 3.463 (1.90), 3.474 (2.20), 3.503 (1.37), 3.524 (0.70), | |
| | 3.550 (0.69), 3.571 (0.74), 4.252 (0.79), 4.302 (0.81), 4.879 (4.81), | |
| | 4.894 (5.51), 7.125 (2.84), 7.137 (3.01), 7.417 (0.91), 7.533 (0.93), | |
| | 7.909 (0.88), 7.992 (16.00), 8.187 (2.17), 8.993 (1.38), 9.004 (1.38), | |
| | 12.253 (1.01). | |
| 225 | 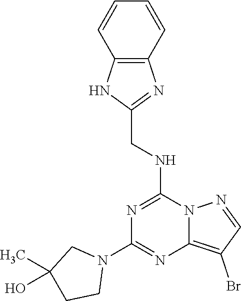 [SMILES: CC1(O)CCN(c2nc(NCc3nc4ccccc4[nH]3)n3ncc(Br)c3n2)C1] | Intermediate 9 and CAS- RN: [921592- 91-8] 75% LC-MS (Method 2): Rₜ = 0.95 min; MS (ESIpos): m/z = 443 [M + H]⁺ |
| | 1-[4-(1H-benzimidazol-2-ylmethylamino)-8-bromo-pyrazolo[1,5- | |
| | a][1,3,5]triazin-2-yl]-3-methyl-pyrrolidin-3-ol | |
| | ¹H-NMR (400 MHz, DMSO-d6) δ [ppm]: −0.008 (1.45), 0.008 (1.55), | |
| | 1.106 (16.00), 1.232 (10.79), 1.295 (10.84), 1.337 (0.53), 1.729 | |
| | (0.78), 1.752 (1.75), 1.772 (2.28), 1.794 (1.60), 1.818 (1.35), 1.909 | |
| | (0.55), 2.290 (0.65), 2.324 (1.05), 2.329 (1.43), 2.334 (1.05), 2.524 | |
| | (4.13), 2.542 (1.88), 2.666 (1.03), 2.671 (1.43), 2.676 (1.05), 3.076 | |
| | (5.03), 3.158 (1.48), 3.187 (1.88), 3.241 (1.70), 3.270 (2.08), 3.395 | |
| | (1.90), 3.425 (1.80), 3.460 (0.80), 3.496 (2.85), 3.526 (2.43), 3.560 | |
| | (0.98), 3.589 (1.05), 4.726 (4.98), 4.734 (5.21), 4.881 (4.91), 7.100 | |
| | (1.03), 7.115 (3.26), 7.124 (3.78), 7.130 (5.58), 7.137 (4.08), 7.146 | |
| | (3.61), 7.161 (1.15), 7.399 (3.71), 7.406 (2.35), 7.416 (3.38), 7.421 | |
| | (2.95), 7.532 (3.10), 7.549 (2.98), 7.987 (15.45), 8.966 (1.53), 8.979 | |
| | (1.53), 8.992 (1.55), 12.251 (4.93). | |
| 226 | 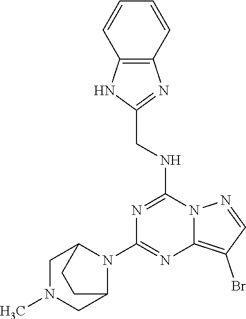 [SMILES: CN1CC2CCC(C1)N2c1nc(NCc2nc3ccccc3[nH]2)n2ncc(Br)c2n1] | Intermediate 9 and CAS- RN: [52407- 92-8] 57% LC-MS (Method 2): Rₜ = 1.10 min; MS (ESIpos): m/z = 468 [M + H]⁺ |
| | N-(1H-benzimidazol-2-ylmethyl)-8-bromo-2-(3-methyl-3, 8- | |
| | diazabicyclo[3.2.1]octan-8-yl)pyrazolo[1,5-a][1,3,5]triazin-4- | |
| | amine | |
| | ¹H-NMR (400 MHz, DMSO-d6) δ [ppm]: 1.235 (0.67), 1.636 (1.00), | |
| | 1.701 (0.99), 1.726 (1.00), 1.753 (0.99), 1.908 (1.18), 1.919 (16.00), | |
| | 2.077 (0.95), 2.086 (0.94), 2.521 (3.18), 2.525 (2.31), 2.542 (1.37), | |
| | 2.676 (0.56), 4.372 (0.66), 4.506 (0.68), 4.816 (2.55), 4.829 (2.78), | |
| | 7.089 (0.42), 7.102 (1.66), 7.112 (2.15), 7.118 (2.14), 7.125 (2.35), | |
| | 7.135 (1.76), 7.147 (0.43), 7.379 (1.32), 7.395 (1.15), 7.513 (1.24), | |
| | 7.531 (1.13), 8.019 (15.34), 9.102 (0.87), 9.116 (1.73), 9.130 (0.82), | |
| | 12.252 (1.73). | |
| 227 | 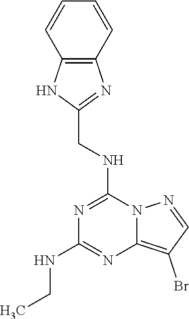 [SMILES: CCNc1nc(NCc2nc3ccccc3[nH]2)n2ncc(Br)c2n1] | Intermediate 9 and CAS- RN: [75-04-7] 80% LC-MS (Method 2): Rₜ = 1.01 min; MS (ESIpos): m/z = 387 [M + H]⁺ |
| | N4-(1H-benzimidazol-2-ylmethyl)-8-bromo-N2-ethyl- | |
| | pyrazolo[1,5-a][1,3,5]triazine-2,4-diamine | |
| | ¹H-NMR (400 MHz, DMSO-d6) δ [ppm]: 0.844 (1.04), 1.081 (2.19), | |
| | 1.098 (3.84), 1.115 (2.26), 2.074 (1.49), 2.523 (5.09), 3.133 (0.60), | |
| | 3.263 (1.50), 3.280 (1.92), 3.294 (1.56), 4.862 (4.28), 7.101 (0.68), | |
| | 7.131 (3.60), 7.162 (0.84), 7.261 (1.19), 7.395 (2.73), 7.412 (2.34), | |
| | 7.530 (2.17), 7.547 (2.08), 7.974 (16.00), 8.866 (0.82), 12.224 | |
| | (1.59). | |
| 228 | 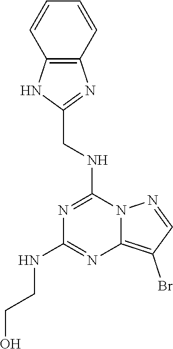 [SMILES: OCCNc1nc(NCc2nc3ccccc3[nH]2)n2ncc(Br)c2n1] | Intermediate 9 and CAS- RN: [141-43-5] 75% LC-MS (Method 2): Rₜ = 0.82 min; MS (ESIpos): m/z = 403 [M + H]⁺ |
| | 2-[(4-{[(1H-benzimidazol-2-yl)methyl]amino}-8- | |
| | bromopyrazolo[1,5-a][1,3,5]triazin-2-yl)amino]ethan-1-ol | |
| | ¹H-NMR (400 MHz, DMSO-d6) δ [ppm]: 2.067 (5.34), 2.517 (16.00), | |
| | 2.522 (11.23), 3.258 (1.40), 3.283 (1.26), 3.327 (5.87), 3.341 (7.08), | |
| | 3.352 (7.56), 3.485 (4.04), 3.500 (5.21), 3.513 (4.37), 4.612 (0.55), | |
| | 4.732 (2.23), 4.850 (6.20), 7.105 (1.12), 7.119 (4.09), 7.123 (3.55), | |
| | 7.129 (5.10), 7.136 (6.17), 7.141 (6.05), 7.152 (5.74), 7.167 (3.37), | |
| | 7.284 (0.58), 7.402 (3.56), 7.418 (3.22), 7.532 (3.59), 7.549 (3.42), | |
| | 7.978 (12.22), 8.904 (0.82), 12.235 (2.35). | |
| 229 | 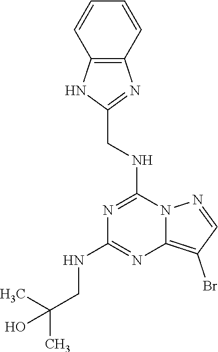 [SMILES: CC(C)(O)CNc1nc(NCc2nc3ccccc3[nH]2)n2ncc(Br)c2n1] | Intermediate 9 and CAS- RN: [2854-16- 2] 46% LC-MS (Method 2): Rₜ = 0.92 min; MS (ESIpos): m/z = 431 [M + H]⁺ |
| | 1-[(4-{[(1H-benzimidazol-2-yl)methyl]amino}-8- | |
| | bromopyrazolo[1,5-a][1,3,5]triazin-2-yl)amino]-2-methylpropan- | |
| | 2-ol | |
| | ¹H-NMR (400 MHz, DMSO-d6) δ [ppm]: −0.003 (4.94), 0.872 (7.89), | |
| | 1.105 (16.00), 1.232 (0.93), 3.162 (1.46), 3.176 (1.56), 3.261 (3.11), | |
| | 3.275 (3.19), 4.414 (1.24), 4.744 (2.56), 4.864 (4.95), 7, 000 (1.39), | |
| | 7.131 (3.56), 7.183 (0.87), 7.404 (1.70), 7.419 (1.67), 7.536 (1.62), | |
| | 7.983 (5.54), 8.949 (0.91), 9.081 (0.52), 12.214 (0.74), 12.266 | |
| | (1.18). | |
| 230 | 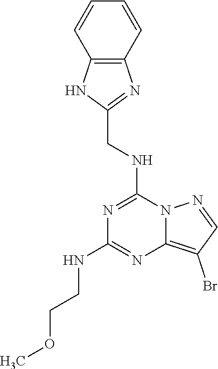 [SMILES: COCCNc1nc(NCc2nc3ccccc3[nH]2)n2ncc(Br)c2n1] | Intermediate 9 and CAS- RN: [109-85-3] 56% LC-MS (Method 2): Rₜ = 0.96 min; MS (ESIpos): m/z = 417 [M + H]⁺ |
| | N4-(1H-benzimidazol-2-ylmethyl)-8-bromo-N2-(2- | |
| | methoxyethyl)pyrazolo[1,5-a][1,3,5]triazine-2,4-diamine | |
| | ¹H-NMR (400 MHz, DMSO-d6) δ [ppm]: −0.004 (7.86), 1.231 (1.33), | |
| | 2.323 (0.74), 2.327 (1.05), 2.331 (0.81), 2.665 (0.81), 2.669 (1.09), | |
| | 2.673 (0.86), 2.897 (2.57), 3.055 (1.14), 3.240 (9.20), 3.297 (0.88), | |
| | 3.441 (5.79), 4.847 (5.18), 4.862 (5.72), 7.134 (3.58), 7.250 (1.35), | |
| | 7.398 (2.43), 7.414 (2.09), 7.530 (1.97), 7.546 (1.79), 7.985 (16.00), | |
| | 8.912 (1.27), 9.088 (0.54), 12.238 (2.94). | |
| 231 | 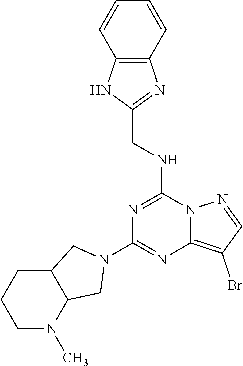 [SMILES: CN1CCCC2CN(c3nc(NCc4nc5ccccc5[nH]4)n4ncc(Br)c4n3)CC21] | Intermediate 9 and CAS- RN: [128740- 18-1] 87% LC-MS (Method 2): Rₜ = 1.13 min; MS (ESIpos): m/z = 482 [M + H]⁺ |
| | N-[(1H-benzimidazol-2-yl)methyl]-8-bromo-2-(1 - | |
| | methyloctahydro-6H-pyrrolo[3,4-b]pyridin-6-yl)pyrazolo[1,5- | |
| | a][1,3,5]triazin-4-amine | |
| | ¹H-NMR (400 MHz, DMSO-d6) δ [ppm]: −0.008 (0.55), 0.000 (16.00), | |
| | 0.008 (0.99), 0.940 (0.83), 0.956 (0.80), 1.383 (1.02), 1.396 (0.94), | |
| | 1.482 (3.05), 1.518 (0.94), 1.589 (1.46), 1.617 (1.15), 1.796 (11.10), | |
| | 1.871 (0.52), 1.895 (0.97), 1.922 (0.57), 1.957 (0.41), 1.978 (0.69), | |
| | 2.004 (0.53), 2.077 (0.44), 2.136 (9.39), 2.262 (0.63), 2.325 (1.30), | |
| | 2.329 (1.52), 2.334 (1.24), 2.388 (1.48), 2.577 (2.07), 2.667 (0.75), | |
| | 2.671 (1.02), 2.676 (0.79), 3.141 (0.88), 3.152 (0.97), 3.172 (1.11), | |
| | 3.183 (1.04), 3.201 (0.68), 3.227 (1.22), 3.253 (0.86), 3.299 (1.07), | |
| | 3.312 (2.09), 3.365 (3.31), 3.394 (1.05), 3.407 (1.40), 3.427 (1.41), | |
| | 3.433 (1.15), 3.453 (0.74), 3.574 (1.43), 3.605 (1.26), 3.716 (1.04), | |
| | 3.747 (0.89), 4.846 (3.01), 4.864 (2.56), 7.102 (1.93), 7.114 (2.95), | |
| | 7.124 (2.92), 7.135 (2.01), 7.377 (1.24), 7.397 (2.01), 7.413 (1.04), | |
| | 7.510 (1.29), 7.526 (2.03), 7.544 (0.99), 7.984 (14.24), 9.025 (0.96), | |
| | 12.267 (2.21). | |
| 232 | 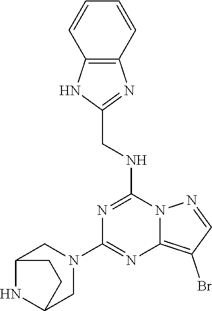 [SMILES: Brc1cnn2c(NCc3nc4ccccc4[nH]3)nc(N3CC4CCC(C3)N4)nc12] | Intermediate 9 and CAS- RN: [17783- 50-5] 9% LC-MS (Method 2): Rₜ = 0.94 min; MS (ESIpos): m/z = 454 [M + H]⁺ |
| | N-(1H-benzimidazol-2-ylmethyl)-8-bromo-2-(3,8- | |
| | diazabicyclo[3.2.1]octan-3-yl)pyrazolo[1,5-a][1,3,5]triazin-4- | |
| | amine | |
| | ¹H-NMR (400 MHz,DMSO-d6) δ [ppm]: 0.835 (0.68), 0.853 (1.15), | |
| | 1.139 (0.84), 1.235 (7.51), 1.395 (1.29), 1.505 (0.94), 1.754 (2.65), | |
| | 1.837 (1.43), 1.907 (1.57), 2.179 (0.84), 2.334 (2.28), 2.406 (1.20), | |
| | 2.542 (6.92), 2.676 (2.24), 2.842 (1.23), 4.097 (0.90), 4.226 (0.92), | |
| | 4.314 (0.88), 4.426 (0.83), 4.831 (10.08), 7.111 (5.26), 7.118 (6.54), | |
| | 7.125 (6.59), 7.132 (6.37), 7.386 (3.41), 7.403 (3.18), 7.526 (3.23), | |
| | 8.001 (9.74), 8.009 (16.00), 8.056 (0.48), 8.239 (0.57), 12.247 | |
| | (4.31). | |
| 233 | 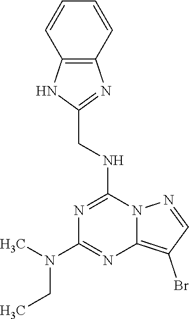 [SMILES: CCN(C)c1nc(NCc2nc3ccccc3[nH]2)n2ncc(Br)c2n1] | Intermediate 9 and CAS- RN: [624-78-2] 31% LC-MS (Method 2): Rₜ = 1.14 min; MS (ESIpos): m/z = 401 [M + H]⁺ |
| | N4-(1H-benzimidazol-2-ylmethyl)-8-bromo-N2-ethyl-N2-methyl- | |
| | pyrazolo[1,5-a][1,3,5]triazine-2,4-diamine | |
| | ¹H-NMR (400 MHz,DMSO-d6) δ [ppm]: 0.754 (1.20), 0.854 (0.48), | |
| | 1.078 (0.57), 1.234 (1.47), 2.077 (0.78), 2.990 (4.67), 3.450 (0.99), | |
| | 4.863 (3.45), 7.092 (0.61), 7.106 (2.24), 7.115 (3.01), 7.122 (3.57), | |
| | 7.128 (3.57), 7.137 (2.82), 7.151 (0.95), 7.387 (1.79), 7.404 (1.74), | |
| | 7.524 (1.98), 7.540 (1.96), 7.995 (16.00), 9.019 (2.09), 12.253 | |
| | (2.64). | |
| 234 | 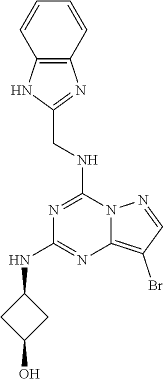 [SMILES: O[C@H]1C[C@@H](Nc2nc(NCc3nc4ccccc4[nH]3)n3ncc(Br)c3n2)C1] | Intermediate 9 and CAS- RN: [1219019- 22-3] 58% LC-MS (Method 2): Rₜ = 0.85 min; MS (ESIpos): m/z = 429 [M + H]⁺ |
| | cis-3-[(4-{[(1H-benzimidazol-2-yl)methyl]amino}-8- | |
| | bromopyrazolo[1,5-a][1,3,5]triazin-2-yl)amino]cyclobutan-1-ol | |
| | ¹H-NMR (400 MHz, DMSO-d6) δ [ppm]: 1.234 (0.69), 1.632 (0.57), | |
| | 1.654 (0.63), 1.755 (0.72), 1.772 (1.14), 1.793 (1.20), 2.077 (2.20), | |
| | 2.269 (0.62), 2.542 (3.87), 3.639 (0.48), 3.834 (0.93), 3.854 (0.95), | |
| | 3.873 (0.72), 4.864 (4.57), 4.951 (0.47), 5.009 (0.95), 5.022 (0.95), | |
| | 7.131 (2.55), 7.143 (2.79), 7.417 (1.04), 7.519 (1.54), 7.538 (2.12), | |
| | 7.639 (0.51), 7.657 (0.50), 7.973 (16.00), 8.881 (0.60), 12.222 | |
| | (0.93). | |
| 235 | 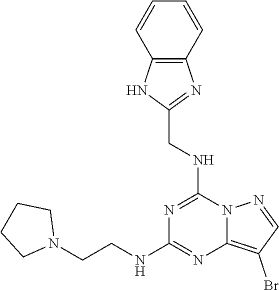 [SMILES: Brc1cnn2c(NCc3nc4ccccc4[nH]3)nc(NCCN3CCCC3)nc12] | Intermediate 9 and CAS- RN: [7154-73- 6] 56% LC-MS (Method 2): Rₜ = 1.06 min; MS (ESIpos): m/z = 456 [M + H]⁺ |
| | N4-(1H-benzimidazol-2-ylmethyl)-8-bromo-N2-(2-pyrrolidin-1- | |
| | ylethyl)pyrazolo[1,5-a][1,3,5]triazine-2,4-diamine | |
| | ¹H-NMR (400 MHz,DMSO-d6) δ [ppm]: 1.234 (0.42), 1.420 (1.52), | |
| | 1.650 (2.68), 2.065 (1.48), 2.206 (0.74), 2.453 (2.65), 2.520 (4.13), | |
| | 2.525 (2.94), 2.542 (1.60), 2.554 (1.61), 2.570 (0.89), 3.201 (0.60), | |
| | 3.377 (1.47), 4.861 (2.78), 7.127 (2.28), 7.324 (0.43), 7.403 (0.63), | |
| | 7.529 (0.59), 7.982 (16.00), 8.888 (0.43), 12.244 (0.84). | |
| 236 | 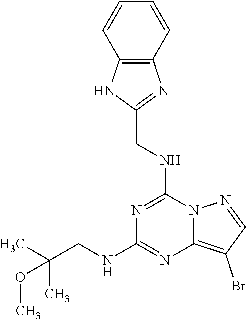 [SMILES: COC(C)(C)CNc1nc(NCc2nc3ccccc3[nH]2)n2ncc(Br)c2n1] | Intermediate 9 and CAS- RN: [89282- 70-2] 50% LC-MS (Method 2): Rₜ = 1.07 min; MS (ESIpos): m/z = 446 [M + H]⁺ |
| | N4-(1H-benzimidazol-2-ylmethyl)-8-bromo-N2-(2-methoxy-2- | |
| | methyl-propyl)pyrazolo[1,5-a][1,3,5]triazine-2,4-diamine | |
| | ¹H-NMR (400 MHz, DMSO-d6) δ [ppm]: 0.789 (8.87), 1.118 (16.00), | |
| | 1.234 (1.14), 1.754 (0.63), 2.076 (0.66), 2.320 (0.42), 2.520 (4.96), | |
| | 2.525 (3.38), 2.542 (5.80), 2.884 (5.01), 3.134 (9.18), 3.173 (1.67), | |
| | 3.187 (1.67), 3.358 (3.82), 3.374 (2.97), 4.863 (4.73), 6.928 (1.30), | |
| | 7.134 (3.84), 7.385 (0.84), 7.408 (1.24), 7.425 (1.04), 7.538 (1.40), | |
| | 7.555 (1.03), 7.983 (5.28), 8.928 (0.78), 9.071 (0.46), 12.265 (3.66). | |
| 237 | 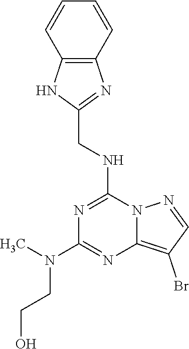 [SMILES: CN(CCO)c1nc(NCc2nc3ccccc3[nH]2)n2ncc(Br)c2n1] | Intermediate 9 and CAS- RN: [109-83-1] 43% LC-MS (Method 2): Rₜ = 0.93 min; MS (ESIpos): m/z = 417 [M + H]⁺ |
| | 2-[(4-{[(1H-benzimidazol-2-yl)methyl]amino}-8- | |
| | bromopyrazolo[1,5-a][1,3,5]triazin-2-yl)(methyl)amino]ethan-1-ol | |
| | ¹H-NMR (400 MHz, DMSO-d6) δ [ppm]: 1.235 (1.56), 2.077 (2.40), | |
| | 3.023 (1.85), 3.113 (4.49), 3.162 (0.45), 3.174 (0.41), 3.403 (1.93), | |
| | 3.575 (2.89), 4.691 (1.49), 4.865 (3.56), 7.101 (0.86), 7.115 (3.17), | |
| | 7.119 (2.73), 7.125 (3.92), 7.131 (4.90), 7.137 (4.51), 7.143 (3.10), | |
| | 7.147 (3.83), 7.161 (1.15), 7.402 (2.82), 7.418 (2.55), 7.423 (2.43), | |
| | 7.531 (2.67), 7.548 (2.62), 7.995 (16.00), 9.011 (1.38), 9.025 (2.74), | |
| | 9.039 (1.44), 12.185 (1.06), 12.267 (0.54). | |
| 238 | 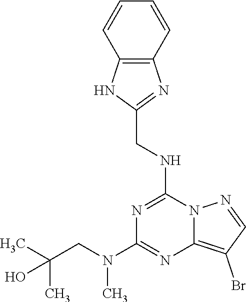 [SMILES: CN(CC(C)(C)O)c1nc(NCc2nc3ccccc3[nH]2)n2ncc(Br)c2n1] | Intermediate 9 and CAS- RN: [67622- 86-0] 42% LC-MS (Method 2): Rₜ = 1.05 min; MS (ESIpos): m/z = 445 [M + H]⁺ |
| | 1-[(4-{[(1H-benzimidazol-2-yl)methyl]amino}-8- | |
| | bromopyrazolo[1,5-a][1,3,5]triazin-2-yl)(methyl)amino]-2- | |
| | methylpropan-2-ol | |
| | ¹H-NMR (400 MHz, DMSO-d6) δ [ppm]: 0.777 (16.00), 1.093 (3.47), | |
| | 1.235 (0.81), 1.754 (0.43), 2.077 (0.51), 2.542 (1.98), 3.060 (1.87), | |
| | 3.131 (9.65), 3.436 (4.73), 3.565 (1.10), 4.371 (3.04), 4.808 (2.51), | |
| | 4.818 (2.69), 4.913 (1.29), 7.120 (2.79), 7.127 (2.97), 7.372 (1.16), | |
| | 7.392 (1.31), 7.505 (1.20), 7.522 (1.35), 8.003 (14.04), 9.010 (1.09), | |
| | 12.248 (1.79). | |
| 239 | 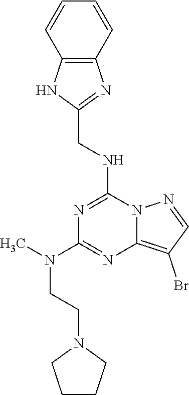 [SMILES: CN(CCN1CCCC1)c1nc(NCc2nc3ccccc3[nH]2)n2ncc(Br)c2n1] | Intermediate 9 and CAS- RN: [327769- 22-0] 65% LC-MS (Method 2): Rₜ = 1.17 min; MS (ESIpos): m/z = 470 [M + H]⁺ |
| | N4-(1H-benzimidazol-2-ylmethyl)-8-bromo-N2-methyl-N2-(2- | |
| | pyrrolidin-1-ylethyl)pyrazolo[1,5-a][1,3,5]triazine-2,4-diamine | |
| | ¹H-NMR (400 MHz, DMSO-d6) δ [ppm]: 1.234 (1.27), 1.358 (7.58), | |
| | 1.634 (2.06), 2.011 (7.31), 2.086 (16.00), 2.228 (3.51), 2.329 (1.96), | |
| | 2.334 (1.42), 2.521 (9.79), 2.525 (6.43), 2.583 (1.19), 2.671 (2.04), | |
| | 2.676 (1.52), 2.990 (2.18), 3.058 (11.36), 3.503 (3.63), 3.658 (1.05), | |
| | 4.862 (6.44), 7.124 (5.63), 7.396 (1.41), 7.516 (1.43), 8.005 (9.44), | |
| | 9.031 (2.21), 12.242 (1.40). | |
| 240 | 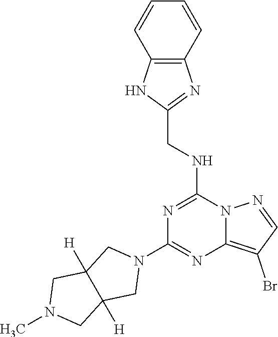 [SMILES: CN1CC2CN(c3nc(NCc4nc5ccccc5[nH]4)n4ncc(Br)c4n3)CC2C1] | Intermediate 9 and CAS- RN: [172739- 03-6] 86% LC-MS (Method 2): Rₜ = 1.06 min; MS (ESIpos): m/z = 468 [M + H]⁺ |
| | N-(1H-benzimidazol-2-ylmethyl)-8-bromo-2-(2-methyl- | |
| | 1,3,3a,4,6,6a-hexahydropyrrolo[3,4-c]pyrrol-5-yl)pyrazolo[1,5- | |
| | a][1,3,5]triazin-4-amine | |
| | ¹H-NMR (400 MHz, DMSO-d6) δ [ppm]: 2.076 (1.97), 2.120 (16.00), | |
| | 2.186 (0.44), 2.222 (0.46), 2.325 (1.36), 2.329 (1.60), 2.334 (1.48), | |
| | 2.373 (1.11), 2.667 (0.60), 2.671 (0.81), 2.676 (0.67), 2.754 (1.16), | |
| | 3.602 (0.81), 4.867 (3.10), 7.115 (1.42), 7.124 (2.06), 7.130 (2.20), | |
| | 7.137 (2.40), 7.146 (1.90), 7.161 (0.60), 7.396 (1.24), 7.413 (1.19), | |
| | 7.528 (1.24), 7.546 (1.20), 7.994 (10.06), 9.021 (0.99), 12.245 | |
| | (1.53). | |

### Example 241

### N-[(1H-benzimidazol-2-yl)methyl]-2-(morpholin-4-yl)-8-(trifluoromethyl) pyrazolo[1,5-a][1,3,5]triazin-4-amine

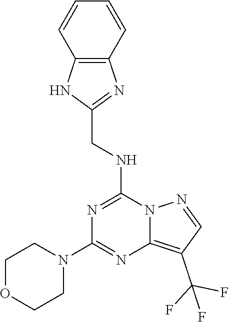 [SMILES: FC(F)(F)c1cnn2c(NCc3nc4ccccc4[nH]3)nc(N3CCOCC3)nc12]

To a solution of 2-(morpholin-4-yl)-8-(trifluoromethyl)-N-[(1-{[2-(trimethylsilyl)ethoxy]methyl}-1H-benzimidazol-2-yl)methyl]pyrazolo[1,5-a][1,3,5]triazin-4-amine (Intermediate 132, 160 mg, 78% purity, 227 μmol) in dichloromethane (4.0 mL) was added trifluoroacetic acid (3.1 mL, 40 mmol; CAS-RN:[76-05-1]) in one portion. The reaction mixture was stirred at rt for 16 h. The reaction mixture was concentrated and purified by preparative HPLC (Instrument: Gilson-281; Column: Phenomenex Synergi C18 150*25*10 μm; eluent A: water (0.225% formic acid), eluent B: acetonitrile; gradient: 0-10 min 18-48% B; flow 25 mL/min; temperature: room temperature; Detector: UV 220/254 nm) to give 58.0 mg (89% purity, 54% yield) of the title compound as an off-white solid.

LC-MS (Method D): Rₜ=0.798 min; MS (ESIpos): m/z=419.5 [M+H]⁺.

¹H-NMR (400 MHz, DMSO-d6) δ[ppm]: 3.44-3.55 (m, 4H), 3.60-3.69 (m, 4H), 4.92 (br d, 2H), 7.18 (dd, 2H), 7.50 (br dd, 2H), 8.26-8.29 (m, 1H), 9.31 (br t, 1H), 12.18-12.79 (m, 1H)

### Example 242

### N-[(1H-benzimidazol-2-yl)methyl]-2-(4-methylpiperazin-1-yl)-8-(pyridin-4-yl)pyrazolo[1,5-a][1,3,5]triazin-4-amine

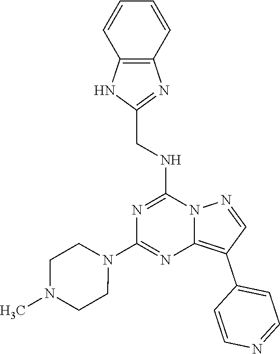 [SMILES: CN1CCN(c2nc(NCc3nc4ccccc4[nH]3)n3ncc(-c4ccncc4)c3n2)CC1]

N-[(4-methoxyphenyl)methyl]-N-({1-[(4-methoxyphenyl)methyl]-1H-benzimidazol-2-yl}methyl)-2-(4-methylpiperazin-1-yl)-8-(pyridin-4-yl)pyrazolo[1,5-a][1,3,5]triazin-4-amine (Intermediate 134, 160 mg, 235 μmol) was dissolved in trifluoracetic acid (4.0 mL, 52 mmol; CAS-RN:[76-05-1]) and the mixture was stirred for 5 h at 150° C. in a microwave. The mixture was concentrated and purified by flash chromatography using amino phase silica gel (dichloromethane-ethanol gradient) to give 62.0 mg (58% yield) of the title compound.

LC-MS (Method 2): Rₜ=0.93 min; MS (ESIpos): m/z=441 [M+H]⁺

¹H-NMR (400 MHz, DMSO-d6) δ[ppm]: 1.234 (0.40), 2.140 (16.00), 2.225 (1.01), 2.320 (0.63), 2.325 (1.04), 2.329 (1.31), 2.334 (0.97), 2.339 (0.54), 2.521 (3.74), 2.525 (2.53), 2.667 (0.77), 2.671 (1.08), 2.676 (0.75), 3.716 (1.38), 4.894 (8.20), 7.113 (0.70), 7.123 (4.24), 7.131 (3.88), 7.138 (4.06), 7.146 (4.58), 7.156 (0.81), 7.475 (1.32), 7.966 (7.25), 7.971 (4.05), 7.978 (4.02), 7.982 (7.42), 8.470 (8.10), 8.474 (4.23), 8.482 (4.01), 8.486 (7.12), 8.629 (12.00).

### Example 243

### N-[(1H-benzimidazol-2-yl)methyl]-2-(4-methylpiperazin-1-yl)-8-(2-methylpyridin-4-yl)pyrazolo[1,5-a][1,3,5]triazin-4-amine

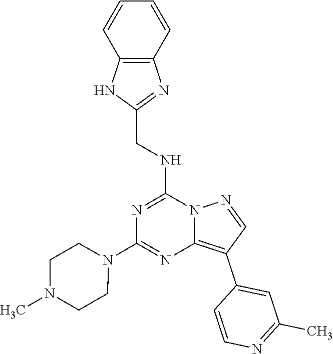

N-[(4-methoxyphenyl)methyl]-N-({1-[(4-methoxyphenyl)methyl]-1H-benzimidazol-2-yl}methyl)-2-(4-methylpiperazin-1-yl)-8-(2-methylpyridin-4-yl)pyrazolo[1,5-a][1,3,5]triazin-4-amine (Intermediate 135, 195 mg, 281 μmol) was dissolved in trifluoracetic acid (4.0 mL, 52 mmol; CAS-RN:[76-05-1]) and the mixture was stirred for 5 h at 150° C. in a microwave. The mixture was concentrated and purified by flash chromatography using amino phase silica gel (dichloromethane-ethanol gradient) to give 44.0 mg (32% yield) of the title compound.

LC-MS (Method 2): Rₜ=0.96 min; MS (ESIneg): m/z=453 [M−H]⁻

¹H-NMR (400 MHz, DMSO-d6) δ[ppm]: 1.234 (0.48), 2.138 (13.47), 2.223 (1.14), 2.299 (0.51), 2.320 (0.60), 2.325 (1.00), 2.329 (1.30), 2.334 (0.96), 2.339 (0.52), 2.458 (16.00), 2.521 (4.43), 2.525 (3.00), 2.667 (0.82), 2.671 (1.12), 2.676 (0.81), 3.567 (0.67), 3.710 (1.31), 4.892 (4.22), 7.124 (1.78), 7.131 (2.00), 7.139 (2.07), 7.146 (1.99), 7.422 (0.48), 7.533 (0.49), 7.812 (3.58), 7.831 (2.00), 7.835 (1.55), 7.845 (1.94), 7.848 (1.68), 8.345 (3.15), 8.358 (2.87), 8.603 (9.97), 9.125 (0.55), 12.292 (0.60).

### Example 244

### N-[(1H-benzimidazol-2-yl)methyl]-8-cyclopentyl-2-(morpholin-4-yl)pyrazolo[1,5-a][1,3,5]triazin-4-amine

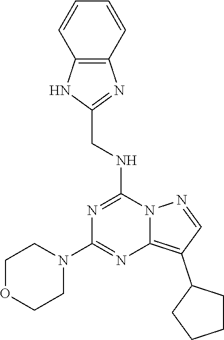 [SMILES: c1ccc2[nH]c(CNc3nc(N4CCOCC4)nc4c(C5CCCC5)cnn34)nc2c1]

To a solution of 8-cyclopentyl-2-(morpholin-4-yl)-N-[(1-{[2-(trimethylsilyl)ethoxy]methyl}-1H-benzimidazol-2-yl)methyl]pyrazolo[1,5-a][1,3,5]triazin-4-amine (Intermediate 139, 100 mg, 182 μmol) in dichloromethane (3.0 mL) was added trifluoroacetic acid (1.0 mL, 13 mmol; CAS-RN:[76-05-1]) in one portion. The reaction mixture was stirred at rt for 16 h. The reaction mixture was concentrated and purified by preparative HPLC (Instrument: Gilson-281; Column: Phenomenex Synergi C18 150*25*10 μm; eluent A: water (0.225% formic acid), eluent B: acetonitrile; gradient: 0-10 min 18-48% B; flow 25 mL/min; temperature: room temperature; Detector: UV 220/254 nm) to give 40.6 mg (94% purity, 50% yield) of the title compound as an off-white solid.

LC-MS (Method D): Rₜ=0.831 min; MS (ESIpos): m/z=420 [M+H]⁺

¹H NMR (400 MHz, DMSO-d₆): δ ppm=1.57-1.78 (m, 6H), 1.92-2.02 (m, 2H), 2.98-3.04 (m, 1H), 3.45-3.48 (m, 4H), 3.51-3.56 (m, 4H), 4.99 (d, 2H), 7.33 (dd, 2H), 7.62 (dd, 2H), 7.83 (s, 1H), 8.98 (br t, 1H)

### Example 245

### 2-[(3S)-3-aminopyrrolidin-1-yl]-N-[(1H-benzimidazol-2-yl)methyl]-8-bromopyrazolo[1,5-a][1,3,5]triazin-4-amine

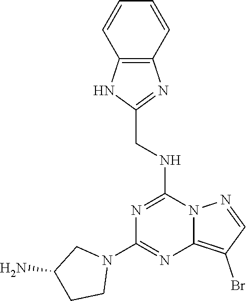 [SMILES: N[C@H]1CCN(c2nc(NCc3nc4ccccc4[nH]3)n3ncc(Br)c3n2)C1]

Tert-butyl [(3S)-1-(4-{[(1H-benzimidazol-2-yl)methyl]amino}-8-bromopyrazolo[1,5-a][1,3,5]triazin-2-yl)pyrrolidin-3-yl]carbamate (Intermediate 140, 254 mg, 66% purity, 318 μmol) was dissolved in dichloromethane, trifluoroacetic acid (120 μL, 1.6 mmol; CAS-RN:[76-05-1]) was added and the mixture was stirred for 18 h at rt. Another 120 μL of trifluoroacetic acid were added and the mixture was stirred over for 72 h at rt. The reaction mixture was treated with aqueous sodiumbicarbonate solution and a precipitate was formed. The precipitate was filtered off and washed with ethylacetate. The filtrate was extracted with ethylacetate and the combined organic layers were concentrated under reduced pressure. The residue was combined with the precipitate and was purified by preparative HPLC to give 25.3 mg (17% yield) of the title compound.

### Preparative HPLC Method: BA

Instrument: Waters Autopurificationsystem; Column: Kinetex C18 Evo 5μ, 150×30 mm; eluent A: water+0.2 vol % aqueous ammonia (32%); eluent B: acetonitril; gradient: 0.0-0.5 min 25% B (35-70 mL/min), 0.5-5.5 min 25-35% B; flow: 70 mL/min; temperature: 25° C.; DAD scan: 210-400 nm

### Analytical HPLC Method: BA

Instrument: Waters Acquity UPLCMS SingleQuad; Column: Acquity UPLC BEH C18 1.7μ, 50×2.1 mm; eluent A: water+0.2 vol % aqueous ammonia (32%); eluent B: acetonitril; gradient: 0-1.6 min 1-99% B, 1.6-2.0 min 99% B; flow: 0.8 mL/min; temperature: 60° C.; DAD scan: 210-400 nm

Analytical HPLC: Rₜ=0.88 min.

Specific Optical Rotation (Method O1): +4.3° (c=10 mg/mL, DMSO)

LC-MS (Method 2): Rₜ=0.89 min; MS (ESIpos): m/z=428 [M+H]⁺

¹H-NMR (400 MHz, DMSO-d6) δ[ppm]: −0.009 (0.31), 0.007 (0.30), 1.895 (0.16), 2.334 (0.24), 2.520 (1.38), 2.524 (0.92), 2.542 (16.00), 2.676 (0.24), 3.163 (0.17), 3.191 (0.18), 3.422 (0.33), 3.436 (0.29), 3.484 (0.47), 3.498 (0.38), 3.538 (0.24), 3.550 (0.22), 3.566 (0.28), 3.579 (0.21), 4.886 (1.76), 7.126 (0.70), 7.135 (0.73), 7.417 (0.21), 7.532 (0.21), 7.986 (3.19).

### Example 246

### N-[(1H-benzimidazol-2-yl)methyl]-8-cyclopropyl-2-(4-methylpiperazin-1-yl)pyrazolo[1,5-a][1,3,5]triazin-4-amine

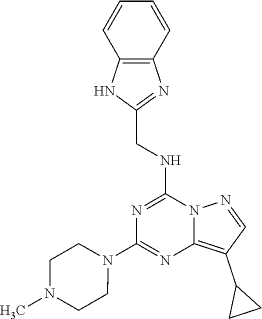 [SMILES: CN1CCN(c2nc(NCc3nc4ccccc4[nH]3)n3ncc(C4CC4)c3n2)CC1]

N-[(1H-benzimidazol-2-yl)methyl]-8-cyclopropyl-2-(methanesulfonyl)pyrazolo[1,5-a][1,3,5]triazin-4-amine (Intermediate 57, 150 mg, 391 μmol) was provided in acetonitrile (3.0 mL), 1-methylpiperazine (220 μL, 2.0 mmol; CAS-RN:[109-01-3]) was added and the reaction mixture was stirred for 2 h at 50° C. and for 3 days at 35° C. The reaction mixture was treated with water, the formed precipitate was filtered off and dried to give 118 mg (73% yield) of the title compound.

LC-MS (Method 2): Rₜ=1.06 min; MS (ESIpos): m/z=404 [M+H]⁺

¹H-NMR (400 MHz, DMSO-d6) δ[ppm]: 0.702 (0.59), 0.714 (1.89), 0.719 (2.34), 0.727 (2.79), 0.732 (2.52), 0.740 (1.14), 0.759 (0.47), 0.765 (1.14), 0.772 (2.21), 0.777 (1.49), 0.786 (1.56), 0.790 (0.96), 0.793 (2.30), 0.798 (1.46), 0.805 (0.48), 0.810 (0.66), 1.716 (0.69), 1.724 (0.71), 1.737 (1.14), 1.746 (0.43), 1.750 (0.67), 1.758 (0.64), 2.111 (9.96), 2.174 (2.30), 2.518 (1.27), 2.523 (0.85), 3.331 (16.00), 4.830 (2.19), 4.844 (2.19), 7.105 (1.08), 7.110 (0.87), 7.116 (1.32), 7.122 (1.48), 7.128 (1.48), 7.133 (0.92), 7.138 (1.17), 7.388 (0.89), 7.405 (0.78), 7.409 (0.72), 7.514 (0.81), 7.518 (0.82), 7.535 (0.77), 7.689 (6.45), 8.787 (0.47), 8.801 (0.92), 8.815 (0.46), 12.236 (1.42).

### Example 247

### N-[(1H-benzimidazol-2-yl)methyl]-8-cyclopropyl-2-(piperazin-1-yl)pyrazolo[1,5-a][1,3,5]triazin-4-amine

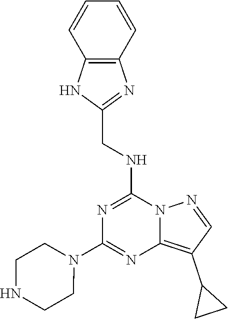 [SMILES: c1ccc2[nH]c(CNc3nc(N4CCNCC4)nc4c(C5CC5)cnn34)nc2c1]

N-[(1H-benzimidazol-2-yl)methyl]-8-cyclopropyl-2-(methanesulfonyl)pyrazolo[1,5-a][1,3,5]triazin-4-amine (Intermediate 57, 150 mg, 391 μmol) was provided in acetonitrile (3.0 mL), piperazine (337 mg, 3.91 mmol; CAS-RN:[110-85-0]) was added and the reaction mixture was stirred for 2 h at 50° C. The reaction mixture was treated with water, the formed precipitate was filtered off and dried to give 114 mg (73% yield) of the title compound.

LC-MS (Method 2): Rₜ=0.90 min; MS (ESIpos): m/z=390 [M+H]⁺

¹H-NMR (400 MHz, DMSO-d6) δ[ppm]: −0.008 (1.02), 0.008 (0.95), 0.703 (1.52), 0.716 (4.78), 0.721 (6.26), 0.728 (6.96), 0.734 (6.01), 0.741 (2.97), 0.750 (1.10), 0.759 (1.65), 0.762 (2.96), 0.770 (5.22), 0.775 (4.57), 0.783 (3.66), 0.788 (2.51), 0.791 (5.79), 0.796 (3.66), 0.804 (1.04), 0.808 (1.60), 1.234 (0.42), 1.699 (0.90), 1.713 (1.74), 1.720 (1.76), 1.734 (2.81), 1.742 (1.08), 1.746 (1.68), 1.755 (1.61), 1.767 (0.77), 2.077 (4.35), 2.334 (0.67), 2.520 (3.05), 2.525 (2.43), 2.555 (6.14), 2.607 (5.21), 2.676 (0.63), 3.507 (5.65), 3.519 (7.36), 3.530 (5.38), 4.831 (12.56), 7.117 (3.33), 7.127 (3.50), 7.406 (0.96), 7.515 (0.96), 7.679 (16.00), 12.236 (0.89).

### Example 248

### N-[(1H-benzimidazol-2-yl)methyl]-8-cyclopropyl-2-[(3R,5S)-3,5-dimethylpiperazin-1-yl]pyrazolo[1,5-a][1,3,5]triazin-4-amine

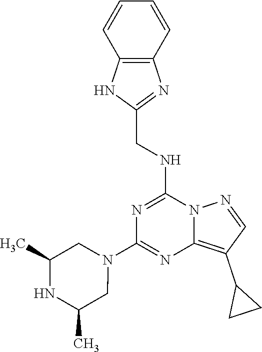 [SMILES: C[C@@H]1CN(c2nc(NCc3nc4ccccc4[nH]3)n3ncc(C4CC4)c3n2)C[C@H](C)N1]

N-[(1H-benzimidazol-2-yl)methyl]-8-cyclopropyl-2-(methanesulfonyl)pyrazolo[1,5-a][1,3,5]triazin-4-amine (Intermediate 57, 150 mg, 391 μmol) was provided in acetonitrile (3.0 mL), cis-2,6-dimethylpiperazine (223 mg, 1.96 mmol; CAS-RN:[108-49-6]) was added and the reaction mixture was stirred for 2 h at 50° C. and for 3 days at 35° C. The reaction mixture was treated with water, the formed precipitate was filtered off and dried to give 118 mg (72% yield) of the title compound.

LC-MS (Method 2): Rₜ=1.05 min; MS (ESIpos): m/z=418 [M+H]⁺

¹H-NMR (400 MHz, DMSO-d6) δ[ppm]: 0.693 (1.62), 0.699 (1.42), 0.705 (4.43), 0.710 (5.54), 0.717 (6.66), 0.722 (5.59), 0.731 (3.03), 0.752 (1.35), 0.767 (3.33), 0.774 (5.42), 0.780 (3.86), 0.788 (3.58), 0.791 (2.79), 0.795 (6.03), 0.801 (4.21), 0.805 (2.13), 0.812 (2.49), 0.863 (4.36), 1.705 (0.89), 1.718 (1.69), 1.725 (1.73), 1.732 (0.87), 1.738 (2.98), 1.747 (1.03), 1.751 (1.60), 1.760 (1.54), 1.772 (0.74), 2.103 (1.54), 2.130 (2.13), 2.158 (1.31), 2.518 (2.08), 2.523 (1.45), 4.326 (1.16), 4.814 (6.10), 7.107 (3.08), 7.121 (3.30), 7.393 (1.05), 7.507 (1.09), 7.667 (16.00), 8.770 (0.85), 12.250 (2.92).

### Example 249

### N-[(1H-benzimidazol-2-yl)methyl]-8-cyclopropyl-2-[(4aS,7aS)-octahydro-6H-pyrrolo[3,4-b]pyridin-6-yl]pyrazolo[1,5-a][1,3,5]triazin-4-amine

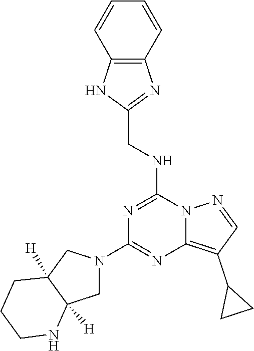 [SMILES: c1ccc2[nH]c(CNc3nc(N4C[C@@H]5CCCN[C@@H]5C4)nc4c(C5CC5)cnn34)nc2c1]

N-[(1H-benzimidazol-2-yl)methyl]-8-cyclopropyl-2-(methanesulfonyl)pyrazolo[1,5-a][1,3,5]triazin-4-amine (Intermediate 57, 150 mg, 391 μmol) was provided in acetonitrile (3.0 mL), (4aS,7aS)-octahydro-1H-pyrrolo[3,4-b]pyridine (148 mg, 1.17 mmol; CAS-RN:[151213-40-0]) was added and the reaction mixture was stirred for 2 h at 50° C. The reaction mixture was treated with water, the formed precipitate was filtered off and dried to give 128 mg (73% yield) of the title compound.

LC-MS (Method 2): Rₜ=1.05 min; MS (ESIpos): m/z=430 [M+H]⁺

¹H-NMR (400 MHz, DMSO-d6) δ[ppm]: −0.008 (3.12), 0.008 (2.51), 0.730 (11.12), 0.753 (5.82), 0.764 (7.67), 0.773 (7.01), 0.782 (8.28), 0.854 (0.52), 1.234 (1.70), 1.298 (2.46), 1.344 (2.22), 1.550 (2.93), 1.573 (3.64), 1.655 (1.80), 1.689 (1.56), 1.702 (2.46), 1.722 (3.93), 1.736 (3.64), 1.743 (2.51), 1.756 (1.75), 1.988 (0.66), 2.008 (0.71), 2.107 (2.22), 2.402 (2.41), 2.427 (4.45), 2.452 (2.84), 2.520 (8.66), 2.525 (5.82), 2.737 (4.07), 2.766 (3.50), 3.138 (2.75), 3.175 (3.27), 3.278 (1.42), 3.305 (3.98), 3.360 (3.98), 3.369 (4.54), 3.380 (6.06), 3.393 (5.96), 3.419 (3.69), 3.444 (2.46), 3.453 (2.37), 3.474 (2.37), 3.500 (0.90), 4.855 (10.22), 7.116 (8.95), 7.130 (9.75), 7.138 (6.06), 7.400 (3.64), 7.417 (3.64), 7.522 (3.83), 7.538 (3.31), 7.644 (16.00), 8.663 (3.64), 12.233 (3.98).

### Example 250

### 2-[(3R)-3-aminopyrrolidin-1-yl]-N-[(1H-benzimidazol-2-yl)methyl]-8-bromopyrazolo[1,5-a][1,3,5]triazin-4-amine

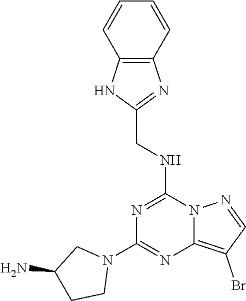 [SMILES: N[C@@H]1CCN(c2nc(NCc3nc4ccccc4[nH]3)n3ncc(Br)c3n2)C1]

Tert-butyl [(3R)-1-(4-{[(1H-benzimidazol-2-yl)methyl]amino}-8-bromopyrazolo[1,5-a][1,3,5]triazin-2-yl)pyrrolidin-3-yl]carbamate (Intermediate 141, 126 mg, 88% purity, 210 μmol) was dissolved in dichloromethane (2.0 mL), trifluoroacetic acid (160 μL, 2.1 mmol; CAS-RN:[76-05-1]) was added and the mixture was stirred for 25 h at rt. The reaction mixture was concentrated, the residue was dissolved in methanol and triethylamine (100 μL) was added. The solution was concentrated under reduce pressure and purified by preparative HPLC to give 58.3 mg (62% yield) of the title compound.

### Preparative HPLC Method: BA

Instrument: Waters Autopurificationsystem; Column: XBrigde C18 5μ, 100×30 mm; eluent A: water+0.2 vol % aqueous ammonia (32%); eluent B: acetonitril; gradient: 0.0-0.5 min 20% B (35-70 mL/min), 0.5-5.5 min 20-35% B; flow: 70 mL/min; temperature: 25° C.; DAD scan: 210-400 nm

### Analytical HPLC Method: BA

Instrument: Waters Acquity UPLCMS SingleQuad; Column: Acquity UPLC BEH C18 1.7μ, 50×2.1 mm; eluent A: water+0.2 vol % aqueous ammonia (32%); eluent B: acetonitril; gradient: 0-1.6 min 1-99% B, 1.6-2.0 min 99% B; flow: 0.8 mL/min; temperature: 60° C.; DAD scan: 210-400 nm

Analytical HPLC: Rₜ=0.87 min.

Specific Optical Rotation (Method O1): −7.6° (c=10 mg/mL, DMSO)

LC-MS (Method 2): Rₜ=0.88 min; MS (ESIpos): m/z=428 [M+H]⁺

¹H-NMR (400 MHz, DMSO-d6) δ[ppm]: −0.008 (1.07), 0.008 (1.00), 1.753 (0.66), 2.031 (0.59), 2.520 (4.07), 2.525 (2.54), 2.542 (6.41), 3.258 (0.91), 3.338 (1.09), 3.423 (1.27), 3.505 (1.61), 3.521 (1.66), 3.565 (2.36), 3.578 (2.36), 3.613 (2.43), 3.627 (2.32), 4.892 (8.84), 7.109 (0.84), 7.120 (5.57), 7.127 (4.84), 7.135 (5.11), 7.142 (6.07), 7.153 (1.02), 7.466 (2.70), 7.474 (2.75), 7.480 (2.66), 7.488 (2.37), 8.007 (13.95), 8.305 (16.00).

### Example 251

### 2-[(7S)-7-amino-5-azaspiro[2.4]heptan-5-yl]-N-[(1H-benzimidazol-2-yl)methyl]-8-bromopyrazolo[1,5-a][1,3,5]triazin-4-amine

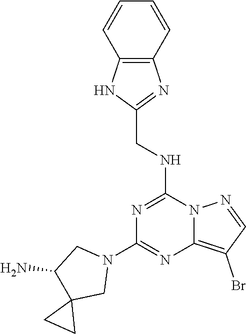 [SMILES: N[C@@H]1CN(c2nc(NCc3nc4ccccc4[nH]3)n3ncc(Br)c3n2)CC12CC2]

Tert-butyl [(7S)-5-(4-{[(1H-benzimidazol-2-yl)methyl]amino}-8-bromopyrazolo[1,5-a][1,3,5]triazin-2-yl)-5-azaspiro[2.4]heptan-7-yl]carbamate (Intermediate 142, 134 mg, 88% purity, 213 μmol) was dissolved in dichloromethane (2.0 mL), trifluoroacetic acid (160 μL, 2.1 mmol; CAS-RN:[76-05-1]) was added and the mixture was stirred for 42 h at rt. The reaction mixture was concentrated, the residue was dissolved in methanol and triethylamine (100 μL) was added. The solution was concentrated under reduce pressure and purified by preparative HPLC to give 52.2 mg (48% yield) of the title compound.

### Preparative HPLC Method: BA

Instrument: Waters Autopurificationsystem; Column: XBrigde C18 5μ, 100×30 mm; eluent A: water+0.2 vol % aqueous ammonia (32%); eluent B: acetonitril; gradient: 0.0-0.5 min 20% B (35-70 mL/min), 0.5-5.5 min 20-45% B; flow: 70 mL/min; temperature: 25° C.; DAD scan: 210-400 nm

### Analytical HPLC Method: BA

Instrument: Waters Acquity UPLCMS SingleQuad; Column: Acquity UPLC BEH C_{18 1.7}μ, 50×2.1 mm; eluent A: water+0.2 vol % aqueous ammonia (32%); eluent B: acetonitril; gradient: 0-1.6 min 1-99% B, 1.6-2.0 min 99% B; flow: 0.8 mL/min; temperature: 60° C.; DAD scan: 210-400 nm

Analytical HPLC: Rₜ=0.96 min.

Specific Optical Rotation (Method O1): −16.2° (c=10 mg/mL, DMSO)

LC-MS (Method 2): Rₜ=0.97 min; MS (ESIpos): m/z=454 [M+H]⁺

¹H-NMR (400 MHz, DMSO-d6) δ[ppm]: −0.008 (0.34), 0.008 (0.30), 0.354 (0.17), 0.409 (0.17), 0.471 (0.20), 0.481 (0.24), 0.503 (0.23), 0.514 (0.23), 0.546 (0.19), 0.711 (0.23), 0.725 (0.20), 0.970 (0.17), 1.109 (16.00), 1.674 (0.20), 1.774 (0.18), 1.813 (0.19), 2.324 (0.20), 2.329 (0.27), 2.334 (0.20), 2.520 (1.10), 2.525 (0.70), 2.542 (0.47), 2.666 (0.20), 2.671 (0.28), 2.676 (0.20), 3.033 (0.39), 3.047 (0.41), 3.062 (0.17), 3.154 (0.17), 3.172 (0.32), 3.182 (0.23), 3.200 (0.38), 3.245 (0.18), 3.257 (0.17), 3.274 (0.21), 3.286 (0.22), 3.369 (0.43), 3.399 (0.60), 3.428 (0.26), 3.544 (0.32), 3.573 (0.26), 3.642 (0.18), 3.657 (0.21), 3.673 (0.26), 3.686 (0.24), 3.703 (0.16), 4.195 (0.71), 4.843 (0.89), 4.905 (0.93), 7.126 (0.69), 7.136 (0.72), 7.407 (0.27), 7.533 (0.27), 7.988 (3.65), 7.993 (0.41), 12.241 (0.19).

### Example 252

### N-[(1H-benzimidazol-2-yl)methyl]-8-bromo-2-[(3S)-3-(methylamino)pyrrolidin-1-yl]pyrazolo[1,5-a][1,3,5]triazin-4-amine

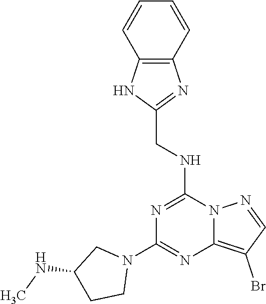 [SMILES: CN[C@H]1CCN(c2nc(NCc3nc4ccccc4[nH]3)n3ncc(Br)c3n2)C1]

Tert-butyl [(3S)-1-(4-{[(1H-benzimidazol-2-yl)methyl]amino}-8-bromopyrazolo[1,5-a][1,3,5]triazin-2-yl)pyrrolidin-3-yl]methylcarbamate (Intermediate 143, 138 mg, 88% purity, 224 μmol) was dissolved in dichloromethane (2.0 mL), trifluoroacetic acid (170 μL, 2.2 mmol; CAS-RN:[76-05-1]) was added and the mixture was stirred for 17 h at rt. The reaction mixture was concentrated, the residue was dissolved in methanol and triethylamine (100 μL) was added. The solution was concentrated under reduced pressure and purified by preparative HPLC to give 52.2 mg (50% yield) of the title compound.

### Preparative HPLC Method: BA

Instrument: Waters Autopurificationsystem; Column: XBrigde C18 5μ, 100×30 mm; eluent A: water+0.2 vol % aqueous ammonia (32%); eluent B: acetonitril; gradient: 0.0-0.5 min 20% B (35-70 mL/min), 0.5-5.5 min 20-45% B; flow: 70 mL/min; temperature: 25° C.; DAD scan: 210-400 nm

### Analytical HPLC Method: BA

Instrument: Waters Acquity UPLCMS SingleQuad; Column: Acquity UPLC BEH C18 1.7μ, 50×2.1 mm; eluent A: water+0.2 vol % aqueous ammonia (32%); eluent B: acetonitril; gradient: 0-1.6 min 1-99% B, 1.6-2.0 min 99% B; flow: 0.8 mL/min; temperature: 60° C.; DAD scan: 210-400 nm

Analytical HPLC: Rₜ=0.93 min.

Specific Optical Rotation (Method O1): +0.87° (c=10 mg/mL, DMSO)

LC-MS (Method 2): Rₜ=0.95 min; MS (ESIpos): m/z=442 [M+H]⁺

¹H-NMR (400 MHz, DMSO-d6) δ[ppm]: −0.008 (0.58), 0.008 (0.56), 1.109 (16.00), 1.708 (0.44), 2.155 (2.90), 2.274 (3.10), 2.334 (0.44), 2.520 (2.35), 2.525 (1.49), 2.542 (2.63), 2.676 (0.43), 3.105 (0.46), 3.187 (0.76), 3.201 (0.80), 3.436 (1.58), 3.464 (1.30), 3.504 (0.70), 3.554 (0.53), 3.569 (0.47), 3.583 (0.42), 4.878 (5.67), 7.116 (2.06), 7.123 (2.09), 7.131 (2.14), 7.139 (2.26), 7.468 (0.54), 7.990 (8.82), 8.279 (1.54).

### Example 253

### N-[(1H-benzimidazol-2-yl)methyl]-8-bromo-2-[(3R)-3-(methylamino)pyrrolidin-1-yl]pyrazolo[1,5-a][1,3,5]triazin-4-amine

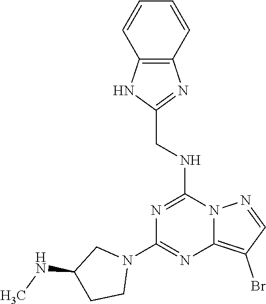 [SMILES: CN[C@@H]1CCN(c2nc(NCc3nc4ccccc4[nH]3)n3ncc(Br)c3n2)C1]

Tert-butyl [(3R)-1-(4-{[(1H-benzimidazol-2-yl)methyl]amino}-8-bromopyrazolo[1,5-a][1,3,5]triazin-2-yl)pyrrolidin-3-yl]methylcarbamate (Intermediate 144, 152 mg, 89% purity, 249 μmol) was dissolved in dichloromethane (2.0 mL), trifluoroacetic acid (170 μL, 2.2 mmol; CAS-RN:[76-05-1]) was added and the mixture was stirred for 17 h at rt. The reaction mixture was concentrated, the residue was dissolved in methanol and triethylamine (100 μL) was added. The solution was concentrated under reduced pressure and purified by preparative HPLC to give 52.7 mg (45% yield) of the title compound.

### Preparative HPLC Method: BA

Instrument: Waters Autopurificationsystem; Column: XBrigde C18 5μ, 100×30 mm; eluent A: water+0.2 vol % aqueous ammonia (32%); eluent B: acetonitril; gradient: 0.0-0.5 min 20% B (35-70 mL/min), 0.5-5.5 min 20-45% B; flow: 70 mL/min; temperature: 25° C.; DAD scan: 210-400 nm

### Analytical HPLC Method: BA

Instrument: Waters Acquity UPLCMS SingleQuad; Column: Acquity UPLC BEH C18 1.7μ, 50×2.1 mm; eluent A: water+0.2 vol % aqueous ammonia (32%); eluent B: acetonitril; gradient: 0-1.6 min 1-99% B, 1.6-2.0 min 99% B; flow: 0.8 mL/min; temperature: 60° C.; DAD scan: 210-400 nm

Analytical HPLC: Rₜ=0.93 min.

Specific Optical Rotation (Method O1): +0.83° (c=10 mg/mL, DMSO)

LC-MS (Method 2): Rₜ=0.95 min; MS (ESIneg): m/z=440 [M−H]⁻

¹H-NMR (400 MHz, DMSO-d6) δ[ppm]: −0.008 (0.68), 0.008 (0.66), 1.109 (16.00), 1.685 (0.45), 1.902 (0.41), 2.134 (3.40), 2.254 (3.58), 2.334 (0.42), 2.520 (2.32), 2.525 (1.46), 2.542 (1.07), 2.676 (0.41), 3.066 (0.43), 3.149 (0.75), 3.176 (0.50), 3.269 (0.50), 3.279 (0.49), 3.433 (0.85), 3.448 (0.87), 3.475 (0.54), 3.497 (0.47), 3.540 (0.42), 4.195 (0.81), 4.876 (5.31), 7.120 (1.71), 7.134 (1.80), 7.397 (0.82), 7.413 (0.80), 7.526 (0.89), 7.544 (0.78), 7.987 (8.55), 12.247 (0.82).

### Example 254

### N-[(1H-benzimidazol-2-yl)methyl]-8-cyclopropyl-2-[(1S,4S)-2-oxa-5-azabicyclo[2.2.1]heptan-5-yl]pyrazolo[1,5-a][1,3,5]triazin-4-amine

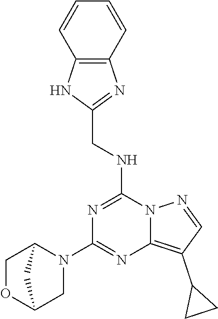 [SMILES: c1ccc2[nH]c(CNc3nc(N4C[C@@H]5C[C@H]4CO5)nc4c(C5CC5)cnn34)nc2c1]

N-[(1H-benzimidazol-2-yl)methyl]-8-cyclopropyl-2-(methanesulfonyl)pyrazolo[1,5-a][1,3,5]triazin-4-amine (Intermediate 57, 59.4 mg, 155 μmol) was provided in acetonitrile (3.0 mL), (1S,4S)-2-oxa-5-azabicyclo[2.2.1]heptane hydrogen chloride (1/1) (21.0 mg, 155 μmol; CAS-RN:[31560-06-2]) and N,N-diisopropylethylamine (27 μL, 150 μmol; CAS-RN:[7087-68-5]) were added and the reaction mixture was stirred for 2 h at 50° C., for 3 days at 35° C. and 7 h at 70° C. Another 7 mg of (1S,4S)-2-oxa-5-azabicyclo[2.2.1]heptane hydrogen chloride (1/1) were added and the reaction mixture was stirred over night at 50° C. The reaction mixture was treated with water, the formed precipitate was filtered off and dried to give 40.1 mg (61% yield) of the title compound.

LC-MS (Method 2): Rₜ=1.01 min; MS (ESIpos): m/z=403 [M+H]⁺

¹H-NMR (400 MHz, DMSO-d6) δ[ppm]: −0.008 (0.79), 0.008 (0.79), 0.701 (1.21), 0.713 (3.85), 0.717 (5.12), 0.725 (5.23), 0.730 (5.07), 0.738 (2.22), 0.759 (1.11), 0.773 (3.49), 0.794 (3.33), 0.937 (1.06), 0.953 (1.06), 1.735 (1.58), 1.771 (1.53), 2.076 (3.91), 2.334 (1.11), 2.520 (5.44), 2.525 (3.43), 2.671 (1.58), 2.676 (1.11), 3.181 (0.53), 3.236 (1.16), 3.363 (1.64), 4.534 (3.75), 4.680 (0.69), 4.826 (3.43), 4.837 (3.33), 7.089 (0.69), 7.103 (2.48), 7.108 (2.22), 7.113 (3.01), 7.120 (3.70), 7.126 (3.27), 7.132 (2.32), 7.136 (2.64), 7.150 (0.79), 7.387 (1.85), 7.405 (1.69), 7.515 (2.01), 7.533 (1.90), 7.685 (16.00), 8.835 (0.95), 12.224 (3.33).

### Example 255

### trifluoroacetic acid-N-[(1H-benzimidazol-2-yl)methyl]-8-bromo-2-[(1R,5R)-3,6-diazabicyclo[3.2.0]heptan-3-yl]pyrazolo[1,5-a][1,3,5]triazin-4-amine (1/1)

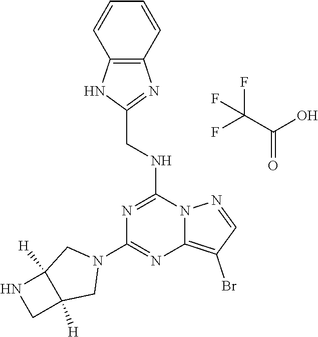 [SMILES: Brc1cnn2c(NCc3nc4ccccc4[nH]3)nc(N3C[C@H]4CN[C@H]4C3)nc12 ; O=C(O)C(F)(F)F]

Tert-butyl (1S,5R)-3-(4-{[(1H-benzimidazol-2-yl)methyl]amino}-8-bromopyrazolo[1,5-a][1,3,5]triazin-2-yl)-3,6-diazabicyclo[3.2.0]heptane-6-carboxylate (Intermediate 151, 111 mg, 84% purity, 173 μmol) was provided in dichloromethane (2.0 mL). Trifluoroacetic acid (130 μL, 1.7 mmol; CAS-RN:[76-05-1]) was added and the mixture was stirred for 20 h at rt. The reaction mixture was concentrated, the residue was dissolved in methanol and triethylamine (100 μL) was added. The solution was concentrated under reduced pressure and purified by preparative HPLC to give 22.4 mg (21% yield) of the title compound.

### Preparative HPLC Method: AA

Instrument: Waters Autopurificationsystem; Column: Chromatorex C18-DE 10μ, 125×30 mm; eluent A: water+0.1 vol % formic acid; eluent B: acetonitril; gradient: 0.0-0.5 min 5% B (35-70 mL/min), 0.5-5.5 min 5-30% B; flow: 70 mL/min; temperature: 25° C.; DAD scan: 210-400 nm

### Analytical HPLC Method: AA

Instrument: Waters Acquity UPLCMS SingleQuad; Column: Acquity UPLC BEH C18 1.7μ, 50×2.1 mm; eluent A: water+0.1 vol % formic acid; eluent B: acetonitril; gradient: 0-1.6 min 1-99% B, 1.6-2.0 min 99% B; flow: 0.8 mL/min; temperature: 60° C.; DAD scan: 210-400 nm

Analytical HPLC: Rₜ=0.61 min.

Specific Optical Rotation (Method O1): +22.02° (c=10 mg/mL, DMSO)

LC-MS (Method 2): Rₜ=0.92 min; MS (ESIpos): m/z=440 [M+H]⁺

¹H-NMR (400 MHz, DMSO-d6) δ[ppm]: 1.235 (0.82), 1.258 (0.49), 2.086 (0.96), 2.333 (1.48), 2.525 (7.00), 2.542 (13.29), 2.676 (1.49), 3.894 (1.09), 3.915 (1.66), 4.008 (1.94), 4.037 (1.90), 4.256 (2.20), 4.290 (2.04), 4.708 (1.55), 4.722 (2.40), 4.736 (1.48), 4.883 (0.69), 4.935 (5.01), 4.976 (0.76), 7.122 (5.54), 7.130 (5.48), 7.137 (5.71), 7.145 (6.02), 7.154 (1.36), 7.476 (1.89), 8.041 (0.44), 8.088 (16.00), 8.102 (0.60), 8.185 (3.53).

### Example 256

### 2-(4-aminopiperidin-1-yl)-N-[(1H-benzimidazol-2-yl)methyl]-8-bromopyrazolo[1,5-a][1,3,5]triazin-4-amine

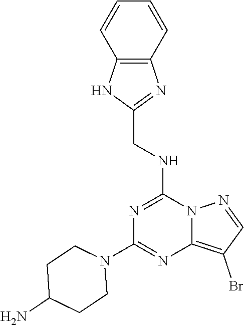 [SMILES: NC1CCN(c2nc(NCc3nc4ccccc4[nH]3)n3ncc(Br)c3n2)CC1]

Tert-butyl [1-(4-{[(1H-benzimidazol-2-yl)methyl]amino}-8-bromopyrazolo[1,5-a][1,3,5]triazin-2-yl)piperidin-4-yl]carbamate (Intermediate 152, 157 mg, 289 μmol) was provided in dichloromethane (2.0 mL). Trifluoroacetic acid (220 μL, 2.9 mmol; CAS-RN:[76-05-1]) was added and the mixture was stirred for 72 h at rt. The reaction mixture was concentrated under vacuum, the residue was solved in methanol and triethylamine (200 μL) was added. The solution was concentrated under reduced pressure and purified by preparative HPLC (Method HT basic) to give 58.7 mg (45% yield) of the title compound.

LC-MS (Method 2): Rₜ=0.92 min; MS (ESIpos): m/z=442 [M+H]⁺

¹H-NMR (400 MHz, DMSO-d6) δ[ppm]: 1.003 (0.83), 1.231 (0.45), 1.603 (0.96), 2.074 (1.66), 2.322 (0.66), 2.327 (0.92), 2.332 (0.67), 2.518 (3.97), 2.523 (2.36), 2.665 (0.69), 2.669 (0.97), 2.673 (0.73), 2.679 (0.53), 2.691 (0.75), 2.706 (0.92), 2.716 (1.40), 2.725 (0.91), 2.740 (0.70), 2.872 (1.12), 2.902 (1.98), 2.930 (1.16), 3.166 (1.22), 4.362 (1.20), 4.854 (10.34), 7.104 (0.81), 7.114 (3.96), 7.122 (3.88), 7.129 (3.98), 7.137 (4.36), 7.147 (0.87), 7.463 (0.86), 7.998 (16.00), 8.007 (0.56).

### Example 257

### N-[(5-methoxy-1H-benzimidazol-2-yl)methyl]-2-(morpholin-4-yl)-8-(trifluoromethyl)pyrazolo[1,5-a][1,3,5]triazin-4-amine

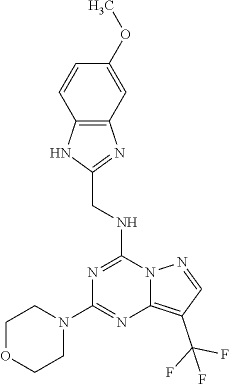 [SMILES: COc1ccc2[nH]c(CNc3nc(N4CCOCC4)nc4c(C(F)(F)F)cnn34)nc2c1]

N-[[5-methoxy-1-(2-trimethylsilylethoxymethyl)benzimidazol-2-yl]methyl]-2-morpholino-8-(trifluoromethyl)pyrazolo[1,5-a][1,3,5]triazin-4-amine; N-[[6-methoxy-1-(2-trimethylsilylethoxymethyl)benzimidazol-2-yl]methyl]-2-morpholino-8-(trifluoromethyl)pyrazolo[1,5-a][1,3,5]triazin-4-amine (1/1) (Intermediate 154, 160 mg, 276 μmol) was provided in dichloromethane (3.6 mL). Trifluoroacetic acid (2.7 mL, 35 mmol; CAS-RN:[76-05-1]) was added and the mixture was stirred for 16 h at rt. The reaction mixture was concentrated under vacuum, the residue was purified by flash chromatography (silica gel, dichloromethane/EtOAc) and dried at 50° C. to give 106 mg (83% yield) of the title compound.

LC-MS (Method 1): Rₜ=0.93 min; MS (ESIpos): m/z=449 [M+H]⁺

¹H-NMR (400 MHz, DMSO-d6) δ[ppm]: 1.232 (0.64), 2.124 (0.41), 2.729 (2.18), 2.889 (2.58), 3.620 (1.40), 3.790 (0.65), 3.802 (16.00), 4.982 (2.00), 4.996 (2.05), 6.954 (0.74), 6.974 (0.79), 7.086 (1.70), 7.091 (1.65), 7.504 (1.30), 7.526 (1.19), 8.287 (4.49), 9.361 (0.59), 9.374 (1.21), 9.387 (0.62).

### Example 258

### 2-(1-amino-3-azabicyclo[3.1.0]hexan-3-yl)-N-(1H-benzimidazol-2-ylmethyl)-8-bromo-pyrazolo[1,5-a][1,3,5]triazin-4-amine

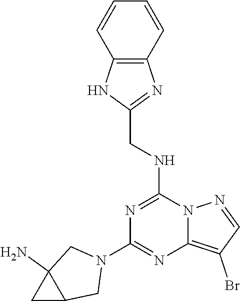 [SMILES: NC12CC1CN(c1nc(NCc3nc4ccccc4[nH]3)n3ncc(Br)c3n1)C2]

Tert-butyl [3-(4-{[(1H-benzimidazol-2-yl)methyl]amino}-8-bromopyrazolo[1′5-a][1,3,5]triazin-2-yl)-3-azabicyclo[3.1.0]hexan-1-yl]carbamate (Intermediate 155, 139 mg, 257 μmol) was provided in dichloromethane (2.0 mL). Trifluoroacetic acid (200 μL, 2.6 mmol; CAS-RN:[76-05-1]) was added and the mixture was stirred for 67 h at rt. The reaction mixture was concentrated under vacuum, the residue was dissolved in methanol and triethylamine (100 μL) was added. The solution was concentrated under reduced pressure and purified by preparative HPLC to give 33.7 mg (29% yield) of the title compound.

### Preparative HPLC Method: BA

Instrument: Waters Autopurificationsystem; Column: Waters XBrigde C18 5μ 100×30 mm; eluent A: water+0.2 vol % ammonia (32%); eluent B: acetonitril; gradient: 0.0-0.5 min 20% B (25-70 mL/min), 0.5-5.5 min 20-40% B; flow: 70 mL/min; DAD scan: 210-400 nm

### Analytical HPLC Method: BA

Instrument: Waters Acquity UPLCMS SingleQuad; Column: Acquity UPLC BEH C18 1.7μ, 50×2.1 mm; eluent A: water 0.2 vol % ammonia (32%); eluent B: acetonitril; gradient: 0-2.6 min 1-99% B, 2.6-3.0 min 99% B; flow: 0.8 mL/min; temperature: 60° C.; DAD scan: 210-400 nm

Analytical HPLC: Rₜ=0.90 min.

LC-MS (Method 2): Rₜ=0.95 min; MS (ESIpos): m/z=440 [M+H]⁺

¹H-NMR (400 MHz, DMSO-d6) δ[ppm]: 0.261 (2.18), 0.807 (1.34), 1.232 (0.87), 1.259 (0.70), 1.269 (0.73), 1.319 (0.70), 1.330 (0.70), 2.318 (0.76), 2.322 (1.37), 2.327 (1.79), 2.331 (1.40), 2.336 (0.76), 2.518 (5.12), 2.523 (3.61), 2.539 (5.06), 2.659 (0.59), 2.664 (1.20), 2.669 (1.68), 2.673 (1.20), 2.678 (0.56), 3.222 (0.87), 3.249 (1.06), 3.280 (1.23), 3.491 (0.50), 3.510 (0.73), 3.519 (0.70), 3.536 (0.98), 3.565 (0.78), 3.602 (1.09), 3.631 (0.70), 3.813 (0.92), 3.840 (0.98), 3.858 (0.95), 3.885 (0.78), 4.854 (1.79), 4.882 (1.65), 4.895 (1.51), 7.103 (0.56), 7.119 (1.99), 7.127 (2.52), 7.133 (3.22), 7.149 (2.18), 7.164 (0.67), 7.401 (1.73), 7.419 (1.59), 7.535 (1.37), 7.993 (16.00), 8.007 (0.50), 12.219 (1.51).

### Example 259

### 2-[(7R)-7-amino-5-azaspiro[2.4]heptan-5-yl]-N-[(1H-benzimidazol-2-yl)methyl]-8-bromopyrazolo[1,5-a][1,3,5]triazin-4-amine

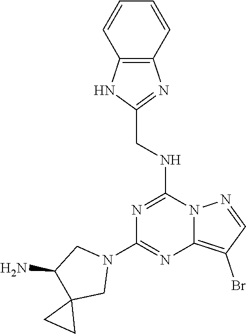 [SMILES: N[C@H]1CN(c2nc(NCc3nc4ccccc4[nH]3)n3ncc(Br)c3n2)CC12CC2]

Tert-butyl [(7R)-5-(4-{[(1H-benzimidazol-2-yl)methyl]amino}-8-bromopyrazolo[1,5-a][1,3,5]triazin-2-yl)-5-azaspiro[2.4]heptan-7-yl]carbamate (Intermediate 156, 151 mg, 71% purity, 194 μmol) was provided in dichloromethane (2.0 mL). Trifluoroacetic acid (150 μL, 1.9 mmol; CAS-RN:[76-05-1]) was added and the mixture was stirred for 120 h at rt. The reaction mixture was concentrated under vacuum, the residue was dissolved in methanol and triethylamine (100 μL) was added. The solution was concentrated under reduced pressure and purified by preparative HPLC to give 44.2 mg (50% yield) of the title compound.

### Preparative HPLC Method: BA

Instrument: Waters Autopurificationsystem; Column: Waters XBrigde C18 5μ 100×30 mm; eluent A: water+0.2 vol % ammonia (32%); eluent B: acetonitril; gradient: 0.0-0.5 min 25% B (25-70 mL/min), 0.5-5.5 min 25-45% B; flow: 70 mL/min; DAD scan: 210-400 nm

### Analytical HPLC Method: BA

Instrument: Waters Acquity UPLCMS SingleQuad; Column: Acquity UPLC BEH C18 1.7μ, 50×2.1 mm; eluent A: water 0.2 vol % ammonia (32%); eluent B: acetonitril; gradient: 0-2.6 min 1-99% B, 2.6-3.0 min 99% B; flow: 0.8 mL/min; temperature: 60° C.; DAD scan: 210-400 nm

Analytical HPLC: Rₜ=0.96 min.

LC-MS (Method 2): Rₜ=0.98 min; MS (ESIpos): m/z=454 [M+H]⁺

¹H-NMR (400 MHz, DMSO-d6) δ[ppm]: 0.229 (0.62), 0.240 (0.59), 0.339 (0.59), 0.351 (0.67), 0.375 (0.42), 0.408 (0.72), 0.419 (0.64), 0.431 (0.50), 0.469 (0.79), 0.480 (0.94), 0.501 (0.87), 0.512 (0.82), 0.544 (0.72), 0.557 (0.52), 0.682 (0.64), 0.708 (0.99), 0.724 (0.89), 0.735 (0.62), 1.137 (0.62), 1.161 (0.42), 1.232 (1.04), 2.084 (0.47), 2.331 (1.07), 2.518 (5.00), 2.522 (3.20), 2.539 (6.12), 2.669 (1.44), 2.673 (1.07), 3.017 (0.62), 3.031 (1.56), 3.045 (1.66), 3.060 (0.72), 3.153 (0.77), 3.169 (1.49), 3.181 (0.94), 3.197 (1.76), 3.245 (0.87), 3.256 (0.89), 3.273 (1.21), 3.285 (1.39), 3.396 (2.65), 3.426 (1.21), 3.542 (1.34), 3.571 (1.04), 3.640 (0.67), 3.654 (0.77), 3.672 (0.99), 3.685 (0.97), 3.701 (0.67), 3.716 (0.57), 4.840 (3.59), 4.902 (3.72), 7.124 (2.70), 7.134 (2.72), 7.403 (1.07), 7.530 (1.07), 7.986 (16.00), 8.000 (0.47), 12.237 (0.74).

### Example 260

### N-(1H-benzimidazol-2-ylmethyl)-8-bromo-2-[(2R or S)-2,4-dimethylpiperazin-1-yl]pyrazolo[1,5-a][1,3,5]triazin-4-amine (Enantiomer 1)

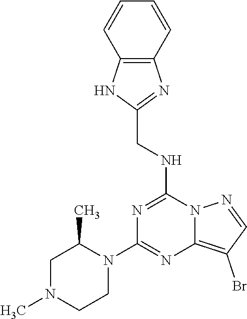 [SMILES: C[C@@H]1CN(C)CCN1c1nc(NCc2nc3ccccc3[nH]2)n2ncc(Br)c2n1]

The title compound from Example 156 (52.0 mg, 114.0 μmol) was separated into enantiomers by preparative chiral HPLC to give 7.6 mg (90% purity) of the title compound (enantiomer 1, Rₜ=10.4-14.3 min) and enantiomer 2 (7.1 mg, Rₜ=14.8-19.5 min, see Example 261).

### Preparative Chiral HPLC Method: NPB

Instrument: PrepCon Labomatic HPLC; Column: Chiralcel OD-H 5μ, 250×20; eluent A: hexane+0.1 vol % diethylamine; eluent B: 2-propanol; isocratic: 80% A+20% B; flow: 15 mL/min; temperature: 25° C.; UV: 280 nm

### Analytical Chiral HPLC Method: NPB

Instrument: Waters Alliance 2695; Column: Chiralcel OD-H 5μ, 100×4.6; eluent A: hexane+0.1 vol % diethylamine; eluent B: 2-propanol; isocratic: 80% A+20% B; flow: 1.4 mL/min; temperature: 25° C.; UV: 280 nm

Analytical chiral HPLC: Rₜ=3.99 min.

Specific Optical Rotation (Method O1): +10.87° (c=10 mg/mL, DMSO)

LC-MS (Method 2): Rₜ=1.10 min; MS (ESIpos): m/z=456 [M+H]⁺

¹H-NMR (400 MHz, DMSO-d6) δ[ppm]: 0.697 (1.13), 0.743 (1.64), 0.761 (1.69), 0.850 (0.83), 0.958 (1.22), 1.027 (4.86), 1.042 (4.80), 1.124 (2.03), 1.143 (4.33), 1.150 (4.71), 1.232 (2.41), 1.293 (0.79), 1.701 (0.73), 1.883 (1.02), 1.891 (1.11), 1.910 (1.17), 1.919 (1.05), 2.106 (16.00), 2.332 (0.96), 2.518 (4.61), 2.523 (3.16), 2.539 (1.71), 2.561 (0.75), 2.673 (1.28), 2.678 (0.94), 2.711 (0.64), 2.927 (0.66), 2.935 (0.73), 2.960 (1.22), 2.967 (1.13), 2.992 (0.73), 2.999 (0.58), 4.282 (1.26), 4.314 (1.17), 4.346 (0.53), 4.356 (0.47), 4.649 (0.47), 4.855 (2.92), 7.091 (0.68), 7.105 (2.28), 7.110 (2.13), 7.114 (2.54), 7.121 (3.97), 7.128 (2.86), 7.133 (2.22), 7.137 (2.43), 7.151 (0.75), 7.380 (2.03), 7.386 (1.26), 7.396 (1.79), 7.401 (1.62), 7.516 (1.88), 7.521 (1.86), 7.538 (1.79), 8.015 (12.42), 9.074 (0.79), 12.252 (2.52).

### Example 261

### N-(1H-benzimidazol-2-ylmethyl)-8-bromo-2-[(2R or S)-2,4-dimethylpiperazin-1-yl]pyrazolo[1,5-a][1,3,5]triazin-4-amine (Enantiomer 2)

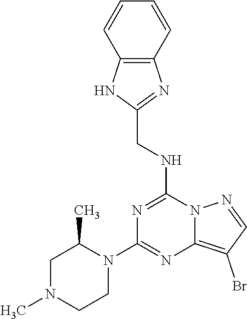 [SMILES: C[C@@H]1CN(C)CCN1c1nc(NCc2nc3ccccc3[nH]2)n2ncc(Br)c2n1]

For the preparation of the racemic title compound see Example 156. Separation of enantiomers by preparative chiral HPLC (method see Example 260) to give 7.1 mg (90% purity) of the title compound (enantiomer 2, Rₜ=14.8-19.5 min).

Analytical chiral HPLC (method see Example 260): Rₜ=4.43 min.

Specific Optical Rotation (Method O1): −7.45° (c=10 mg/mL, DMSO)

LC-MS (Method 2): Rₜ=1.10 min; MS (ESIpos): m/z=456 [M+H]⁺

¹H-NMR (400 MHz, DMSO-d6) δ[ppm]: 0.698 (0.97), 0.758 (0.70), 0.851 (0.87), 0.948 (1.01), 1.027 (3.37), 1.042 (3.37), 1.073 (0.90), 1.092 (1.46), 1.110 (1.11), 1.137 (1.77), 1.147 (1.84), 1.232 (2.71), 1.294 (0.73), 1.700 (0.70), 1.881 (1.01), 1.891 (1.08), 1.904 (1.08), 1.909 (1.15), 1.918 (1.04), 2.106 (16.00), 2.327 (2.09), 2.332 (1.57), 2.518 (7.83), 2.523 (5.18), 2.563 (0.77), 2.669 (2.30), 2.673 (1.84), 2.927 (0.63), 2.935 (0.73), 2.959 (1.22), 2.967 (1.11), 2.991 (0.70), 4.281 (1.22), 4.314 (1.18), 4.648 (0.45), 4.803 (0.45), 4.844 (3.03), 4.855 (3.97), 4.896 (0.56), 7.091 (0.52), 7.105 (1.98), 7.114 (2.54), 7.121 (2.85), 7.128 (2.82), 7.137 (2.23), 7.150 (0.59), 7.380 (1.67), 7.397 (1.43), 7.521 (1.57), 7.537 (1.53), 8.015 (13.11), 12.255 (1.70).

### Example 262

### 8-cyclopentyl-N-[(4,5-difluoro-1H-benzimidazol-2-yl)methyl]-2-(morpholin-4-yl)pyrazolo[1,5-a][1,3,5]triazin-4-amine

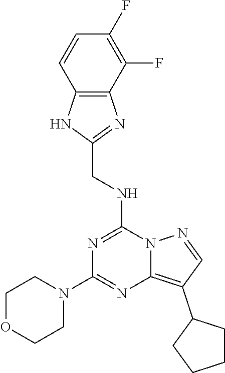 [SMILES: Fc1ccc2[nH]c(CNc3nc(N4CCOCC4)nc4c(C5CCCC5)cnn34)nc2c1F]

To a solution of 8-cyclopentyl-N-[(4,5-difluoro-1-{[2-(trimethylsilyl)ethoxy]methyl}-1H-benzimidazol-2-yl)methyl]-2-(morpholin-4-yl)pyrazolo[1,5-a][1,3,5]triazin-4-amine-8-cyclopentyl-N-[(6,7-difluoro-1-{[2-(trimethylsilyl)ethoxy]methyl}-1H-benzimidazol-2-yl)methyl]-2-(morpholin-4-yl)pyrazolo[1,5-a][1,3,5]triazin-4-amine (1/1) (Intermediate 159, 40.0 mg, 85% purity, 58.1 μmol) in dichloromethane was added trifluoroacetic acid (3.4 mL, 44 mmol; CAS-RN:[76-05-1]) in one portion. The reaction mixture was stirred at rt for 5 h. The reaction mixture was concentrated and purified by preparative HPLC to give 17.0 mg (100% purity, 64% yield) of the title compound as a white solid.

HPLC-Method: Instrument:ACSWH-GX-Q; Column: Waters Xbridge C18 150*25 mm*10 um; eluent A: water (0.225% FA), eluent B: acetonitrile; gradient: 0-10 min 48-78% B; flow 25 mL/min; temperature: RT; Detector: UV 220/254 nm

LC-MS (Method C): Rₜ=0.81 min; MS (ESIpos): m/z=455 [M+H]⁺

¹H-NMR (400 MHz, DMSO-d₆) 5 [ppm]: 1.58-1.83 (m, 6H), 1.92-2.07 (m, 2H), 2.97-3.02 (m, 1H), 3.50-3.57 (m, 4H), 3.58-3.67 (m, 4H), 4.90 (d, 2H), 7.06-7.44 (m, 2H), 7.76 (s, 1H), 8.38-8.83 (m, 1H), 12.30-12.59 (m, 1H)

### Example 263

### N-[(1H-benzimidazol-2-yl)methyl]-8-cyclobutyl-2-(morpholin-4-yl)pyrazolo[1,5-a][1,3,5]triazin-4-amine

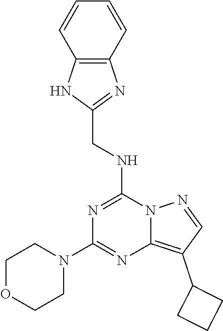 [SMILES: c1ccc2[nH]c(CNc3nc(N4CCOCC4)nc4c(C5CCC5)cnn34)nc2c1]

A solution of 8-cyclobutyl-N-[(4-methoxyphenyl)methyl]-2-morpholino-N-[[1-(2-trimethylsilylethoxymethyl)benzimidazol-2-yl]methyl]pyrazolo[1,5-a][1,3,5]triazin-4-amine (Intermediate 165, 350 mg, 67% purity, 360 μmol) and trifluoromethanesulfonic acid (1.2 mL) in trifluoroacetic acid (6.0 mL) was stirred at 80° C. for 4 hours. The solution was diluted with water, and adjusted to pH=7 by sodium bicarbonate and then extracted with ethyl acetate. The organic phase was washed with brine, dried over anhydrous sodium sulfate and filtered. The filtrate was concentrated and purified by preparative TLC (petroleum ether: ethyl acetate=1:2) to give a yellow oil. The oil was concentrated and purified by preparative HPLC to give 26.4 mg (100% purity, 18% yield) of the title compound as a white solid.

HPLC-Method: Instrument:ACSWH-GX-Q; Column:3_Phenomenex Luna C18 75*30 mm*3 um; eluent A: water (0.225% FA), eluent B: acetonitrile; gradient: 0-8 min 19-39% B; flow 25 mL/min; temperature: RT; Detector: UV 220/254 nm

LC-MS (Method C): Rₜ=0.73 min; MS (ESIpos): m/z=405 [M+H]⁺

¹H-NMR (400 MHz, DMSO-d₆) 5 [ppm]: 1.81-1.98 (m, 2H), 2.20-2.29 (m, 4H), 3.45-3.54 (m, 6H), 3.57-3.62 (m, 4H), 4.86 (d, 2H), 7.13 (dd, 2H), 7.38-7.60 (m, 2H), 7.91 (s, 1H), 8.81-8.90 (m, 1H), 12.13-12.33 (m, 1H)

### Example 264

### 8-cyclobutyl-N-[(4,5-difluoro-1H-benzimidazol-2-yl)methyl]-2-(morpholin-4-yl)pyrazolo[1,5-a][1,3,5]triazin-4-amine

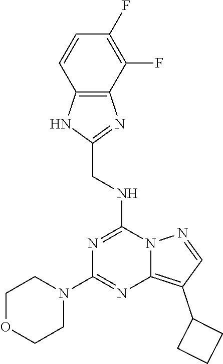 [SMILES: Fc1ccc2[nH]c(CNc3nc(N4CCOCC4)nc4c(C5CCC5)cnn34)nc2c1F]

To a solution of 8-cyclobutyl-N-[(4,5-difluoro-1-{[2-(trimethylsilyl)ethoxy]methyl}-1H-benzimidazol-2-yl)methyl]-N-[(4-methoxyphenyl)methyl]-2-(morpholin-4-yl)pyrazolo[1,5-a][1,3,5]triazin-4-amine (Intermediate 166, 410 mg, 84% purity, 498 μmol) was added trifluoromethanesulfonic (1.2 mL) acid in trifluoroacetic acid (6.0 mL). The mixture was stirred at 80° C. for 4 hours. The solution was diluted with water, and adjusted to pH=7 by sodium bicarbonate and extracted with ethyl acetate. The organic phase was washed with brine, dried over anhydrous sodium sulfate and filtered. The filtrate was concentrated and purified by prepative HPLC to give 77.7 mg (98% purity, 35% yield) of the title compound as a white solid.

HPLC-Method: Instrument:ACSWH-GX-B; Column: Phenomenex luna C18 150*40 mm*15 um; eluent A: water (0.225% FA), eluent B: acetonitrile; gradient: 0-9 min 42-72% B; flow 50 mL/min; temperature: RT; Detector: UV 220/254 nm.

LC-MS (Method C): Rₜ=0.89 min; MS (ESIpos): m/z=441 [M+H]⁺

¹H NMR (400 MHz, DMSO-d₆) 5 [ppm]: 1.83-1.98 (m, 2H), 2.20-2.28 (m, 4H), 2.53-2.54 (m, 1H), 3.46-3.52 (m, 5H), 3.58 (br d, 4H), 4.83-4.92 (m, 2H), 7.14-7.28 (m, 2H), 7.86-7.94 (m, 1H), 8.92 (br s, 1H), 12.62-12.72 (m, 1H)

### Example 265

### formic acid-N-[(1H-benzimidazol-2-yl)methyl]-2-(morpholin-4-yl)-8-(2,2,2-trifluoroethyl)pyrazolo[1,5-a][1,3,5]triazin-4-amine (1/1)

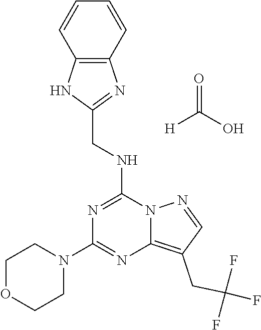 [SMILES: FC(F)(F)Cc1cnn2c(NCc3nc4ccccc4[nH]3)nc(N3CCOCC3)nc12 ; O=CO]

To a solution of N-[(4-methoxyphenyl)methyl]-2-(morpholin-4-yl)-8-(2,2,2-trifluoroethyl)-N-[(1-{[2-(trimethylsilyl)ethoxy]methyl}-1H-benzimidazol-2-yl)methyl]pyrazolo[1,5-a][1,3,5]triazin-4-amine (Intermediate 170, 100 mg, 146 μmol) in trifluoroacetic acid (2.5 mL, 32 mmol; CAS-RN:[76-05-1]) was added trifluoromethanesulfonic acid (500 μL, 5.7 mmol; CAS-RN:[1493-13-6]) at 25° C. The mixture was stirred at 80° C. for 3 h. The mixture was concentrated and the residue was diluted with ethyl acetate. The solution was basified to pH=7 by saturated sodium bicarbonate solution, and the solution was extracted with ethyl acetate and washed with water. The combined organic layers were dried over sodium sulfate, and filtered. The filtrate was concentrated to give a residue. The residue was purified by preparative TLC (dichloromethane:methanol=10:1) followed by preparative HPLC to give 7.20 mg (98% purity, 10% yield) of the title compound as a yellow solid.

HPLC-Method: Instrument:ACSWH-GX-G; Column:Phenomenex Synergi C18 150*25*10 um; eluent A: water (0.225% FA), eluent B: acetonitrile; gradient: 0-9 min 23-53% B; flow 25 mL/min; temperature: RT; Detector: UV 220/254 nm

LC-MS (Method C): Rₜ=0.70 min; MS (ESIpos): m/z=433 [M+H]⁺

¹H-NMR (400 MHz, DMSO-d₆) 5 [ppm]: 3.46-3.49 (m, 5H), 3.58-3.64 (m, 5H), 4.82-4.92 (m, 2H), 7.13 (dd, 2H), 7.47 (br s, 2H), 7.91 (s, 1H), 9.03 (s, 1H), 12.07-12.49 (m, 1H)

### Example 266

### 2-[3-amino-3-(trifluoromethyl)pyrrolidin-1-yl]-N-(1H-benzimidazol-2-ylmethyl)-8-bromo-pyrazolo[1,5-a][1,3,5]triazin-4-amine

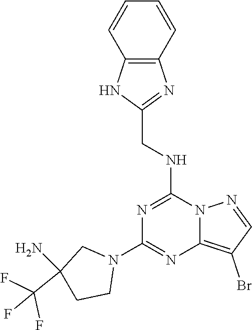 [SMILES: NC1(C(F)(F)F)CCN(c2nc(NCc3nc4ccccc4[nH]3)n3ncc(Br)c3n2)C1]

Tert-butyl N-[1-[4-(1H-benzimidazol-2-ylmethylamino)-8-bromo-pyrazolo[1,5-a][1,3,5]triazin-2-yl]-3-(trifluoromethyl)pyrrolidin-3-yl]carbamate (Intermediate 171, 140 mg, 93% purity, 218 μmol) was provided in dichloromethane (2.0 mL). Trifluoroacetic acid (170 μL, 2.2 mmol; CAS-RN:[76-05-1]) was added and the mixture was stirred for 3 days at rt. Further 170 μL trifluoroacetic acid was added and the mixture was stirred for 3 days at rt. The reaction mixture was concentrated under vacuum, the residue was dissolved in methanol and triethylamine (100 μL) was added. The solution was concentrated under reduced pressure and purified by preparative HPLC (Method HT basic) to give 74.1 mg (68% yield) of the title compound.

LC-MS (Method 2): Rₜ=1.09 min; MS (ESIpos): m/z=496 [M+H]⁺

¹H-NMR (400 MHz, DMSO-d6) δ[ppm]: 1.235 (1.04), 1.833 (0.86), 1.909 (0.67), 2.056 (0.92), 2.076 (16.00), 2.107 (0.77), 2.233 (1.18), 2.324 (2.04), 2.329 (2.75), 2.334 (2.60), 2.338 (2.16), 2.520 (6.90), 2.525 (4.28), 2.666 (1.31), 2.671 (1.81), 2.676 (1.31), 3.161 (1.52), 3.174 (1.48), 3.396 (0.68), 3.426 (1.00), 3.488 (1.28), 3.517 (1.13), 3.566 (3.83), 3.607 (0.65), 3.664 (0.54), 3.687 (0.81), 3.733 (0.54), 3.760 (0.68), 4.892 (3.17), 4.905 (2.73), 7.102 (0.74), 7.120 (2.61), 7.127 (3.27), 7.132 (4.65), 7.139 (3.00), 7.146 (3.23), 7.163 (1.03), 7.400 (1.53), 7.530 (1.72), 7.546 (1.60), 8.027 (14.36), 9.089 (1.10), 12.261 (2.14).

### Example 267

### trifluoroacetic acid-N-[(1H-benzimidazol-2-yl)methyl]-8-bromo-2-[(1S,5S)-3,6-diazabicyclo[3.2.0]heptan-3-yl]pyrazolo[1,5-a][1,3,5]triazin-4-amine (1/1)

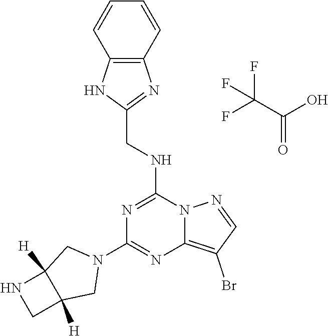 [SMILES: Brc1cnn2c(NCc3nc4ccccc4[nH]3)nc(N3C[C@@H]4CN[C@@H]4C3)nc12 ; O=C(O)C(F)(F)F]

Tert-butyl (1R,5S)-3-(4-{[(1H-benzimidazol-2-yl)methyl]amino}-8-bromopyrazolo[1,5-a][1,3,5]triazin-2-yl)-3,6-diazabicyclo[3.2.0]heptane-6-carboxylate (Intermediate 172, 113 mg, 89% purity, 187 μmol) was provided in dichloromethane (2.0 mL). Trifluoroacetic acid (140 μL, 1.9 mmol; CAS-RN:[76-05-1]) was added and the mixture was stirred for 22 h at rt. The reaction mixture was concentrated under vacuum, the residue was dissolved in methanol and triethylamine (100 μL) was added. The solution was concentrated under reduced pressure and purified by preparative HPLC to give 46.6 mg (43% yield) of the title compound.

### Preparative HPLC Method: AA

Instrument: Waters Autopurificationsystem; Column: Chromatorex C₁₈-DE 10μ, 125×30 mm; eluent A: water+0.1 vol % formic acid; eluent B: acetonitril; gradient: 0.0-0.5 min 5% B (35-70 mL/min), 0.5-5.5 min 5-25% B; flow: 70 mL/min; temperature: 25° C.; DAD scan: 210-400 nm

### Analytical HPLC Method: AA

Instrument: Waters Acquity UPLCMS SingleQuad; Column: Acquity UPLC BEH C18 1.7μ, 50×2.1 mm; eluent A: water+0.1 vol % formic acid; eluent B: acetonitril; gradient: 0-1.6 min 1-99% B, 1.6-2.0 min 99% B; flow: 0.8 mL/min; temperature: 60° C.; DAD scan: 210-400 nm

Analytical HPLC: Rₜ=0.60 Min.

Specific Optical Rotation (Method O1): −21.08° (c=10 mg/mL, DMSO) LC-MS (Method 2): Rₜ=0.92 min; MS (ESIpos): m/z=440 [M+H]⁺

¹H-NMR (400 MHz, DMSO-d6) δ[ppm]: 2.086 (1.20), 2.329 (1.64), 2.334 (1.24), 2.520 (7.42), 2.524 (4.59), 2.542 (5.59), 2.671 (1.70), 2.676 (1.25), 4.007 (1.71), 4.036 (1.19), 4.338 (1.86), 4.374 (1.72), 4.822 (1.24), 4.975 (2.58), 4.989 (1.89), 7.187 (2.70), 7.195 (2.95), 7.202 (3.07), 7.210 (3.08), 7.513 (2.68), 7.522 (2.81), 8.101 (0.57), 8.113 (16.00), 8.128 (0.47), 8.136 (1.36), 8.639 (0.69), 8.862 (0.81), 9.260 (1.10), 9.274 (2.20), 9.289 (1.17).

### Example 268

### N-[(4,5-difluoro-1H-benzimidazol-2-yl)methyl]-2-(morpholin-4-yl)-8-(2,2,2-trifluoroethyl)pyrazolo[1,5-a][1,3,5]triazin-4-amine

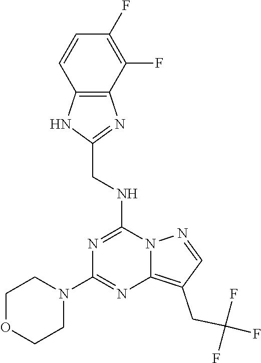 [SMILES: Fc1ccc2[nH]c(CNc3nc(N4CCOCC4)nc4c(CC(F)(F)F)cnn34)nc2c1F]

A solution of N-[(4,5-difluoro-1-{[2-(trimethylsilyl)ethoxy]methyl}-1H-benzimidazol-2-yl)methyl]-N-[(4-methoxyphenyl)methyl]-2-(morpholin-4-yl)-8-(2,2,2-trifluoroethyl)pyrazolo[1,5-a][1,3,5]triazin-4-amine (Intermediate 173, 200 mg, 87% purity, 242 μmol) in trifluoroacetic acid (4.0 mL) was stirred at 80° C. for 4 h. The solution was diluted with water, and adjusted to pH=7 by sodium bicarbonate and then extracted with ethyl acetate. The organic phase was washed with brine, dried over anhydrous sodium sulfate and filtered. The filtrate was concentrated and purified by prepative HPLC to give 58.8 mg (100% purity, 52% yield) of the title compound as a white solid.

HPLC-Method: Instrument:ACSWH-GX-G; Column: Phenomenex Synergi C18 150*25*10 um; eluent A: water (0.225% FA), eluent B: acetonitrile; gradient: 0-9 min 12-42% B; flow 25 mL/min; temperature: RT; Detector: UV 220/254 nm

LC-MS (Method C): Rₜ=0.81 min; MS (ESIpos): m/z=469 [M+H]⁺

¹H-NMR (400 MHz, DMSO-d₆) 5 [ppm]: 3.43-3.55 (m, 6H), 3.61 (br s, 4H), 4.88 (d, 2H), 7.16-7.28 (m, 2H), 7.92 (s, 1H), 9.10 (br s, 1H), 12.62-12.74 (m, 1H)

### Example 269

### N-[(1H-benzimidazol-2-yl)methyl]-8-bromo-2-[(1R,4R)-2,5-diazabicyclo[2.2.1]heptan-2-yl]pyrazolo[1,5-a][1,3,5]triazin-4-amine

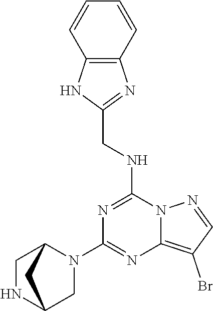 [SMILES: Brc1cnn2c(NCc3nc4ccccc4[nH]3)nc(N3C[C@H]4C[C@@H]3CN4)nc12]

Tert-butyl (1R,4R)-5-(4-{[(1H-benzimidazol-2-yl)methyl]amino}-8-bromopyrazolo[1,5-a][1,3,5]triazin-2-yl)-2,5-diazabicyclo[2.2.1]heptane-2-carboxylate (Intermediate 174, 140 mg, 259 μmol) was provided in dichloromethane (2.0 mL). Trifluoroacetic acid (170 μL, 2.2 mmol; CAS-RN:[76-05-1]) was added and the mixture was stirred for 71 h at rt. The reaction mixture was concentrated under vacuum, the residue was dissolved in methanol and triethylamine (100 μL) was added. The solution was concentrated under reduced pressure and purified by preparative HPLC (Method HT basic) to give 59.5 mg (52% yield) of the title compound.

Specific Optical Rotation (Method O1): −58.12° (c=10 mg/mL, DMSO)

LC-MS (Method 2): Rₜ=0.87 min; MS (ESIpos): m/z=440 [M+H]⁺

¹H-NMR (400 MHz, DMSO-d6) δ[ppm]: 0.854 (0.45), 1.235 (2.12), 1.488 (1.63), 1.510 (2.20), 1.578 (0.84), 1.617 (2.56), 1.642 (2.22), 1.673 (0.82), 1.892 (0.77), 2.330 (3.58), 2.357 (2.49), 2.451 (2.86), 2.526 (6.41), 2.542 (3.37), 2.667 (1.61), 2.672 (2.12), 2.676 (1.54), 2.781 (0.99), 2.805 (1.33), 2.885 (1.26), 2.908 (0.90), 3.209 (1.62), 3.234 (2.53), 3.278 (4.80), 3.557 (4.79), 4.581 (4.02), 4.736 (2.08), 4.839 (9.27), 4.873 (4.76), 7.127 (5.80), 7.405 (2.12), 7.530 (2.17), 7.988 (16.00), 12.263 (2.05).

### Example 270

### N-[(1H-benzimidazol-2-yl)methyl]-8-bromo-2-[(1S,4S)-2,5-diazabicyclo[2.2.1]heptan-2-yl]pyrazolo[1,5-a][1,3,5]triazin-4-amine

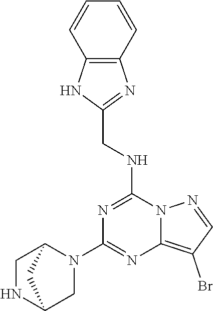 [SMILES: Brc1cnn2c(NCc3nc4ccccc4[nH]3)nc(N3C[C@@H]4C[C@H]3CN4)nc12]

Tert-butyl (1S,4S)-5-(4-{[(1H-benzimidazol-2-yl)methyl]amino}-8-bromopyrazolo[1,5-a][1,3,5]triazin-2-yl)-2,5-diazabicyclo[2.2.1]heptane-2-carboxylate (Intermediate 175, 140 mg, 259 μmol) was provided in dichloromethane (2.4 mL). Trifluoroacetic acid (200 μL, 2.6 mmol; CAS-RN:[76-05-1]) was added and the mixture was stirred for 71 h at rt. The reaction mixture was concentrated under vacuum, the residue was dissolved in methanol and triethylamine (100 μL) was added. The solution was concentrated under reduced pressure and purified by preparative HPLC (Method HT basic) to give 69.7 mg (60% yield) of the title compound.

Specific Optical Rotation (Method O1): +60.18° (c=10 mg/mL, DMSO)

LC-MS (Method 2): Rₜ=0.87 min; MS (ESIpos): m/z=440 [M+H]⁺

¹H-NMR (400 MHz, DMSO-d6) δ[ppm]: 1.234 (1.58), 1.486 (1.63), 1.509 (2.20), 1.577 (0.81), 1.614 (2.55), 1.640 (2.16), 1.672 (0.80), 1.754 (0.44), 1.889 (1.11), 2.329 (3.72), 2.355 (2.68), 2.450 (2.92), 2.520 (7.86), 2.525 (5.11), 2.542 (3.76), 2.667 (1.44), 2.671 (1.89), 2.676 (1.36), 2.778 (1.05), 2.802 (1.45), 2.882 (1.36), 2.905 (0.94), 3.205 (1.47), 3.231 (2.25), 3.275 (3.93), 3.438 (0.94), 3.553 (4.80), 4.579 (4.01), 4.734 (2.05), 4.838 (9.61), 4.873 (4.90), 7.127 (5.65), 7.405 (2.04), 7.531 (2.07), 7.987 (16.00), 12.263 (2.09).

### Example 271

### 2-(2,3,3a,4,6,6a-hexahydro-1H-pyrrolo[2,3-c]pyrrol-5-yl)-N-(1H-benzimidazol-2-ylmethyl)-8-bromo-pyrazolo[1,5-a][1,3,5]triazin-4-amine

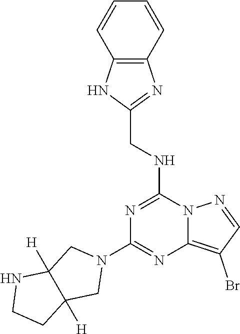 [SMILES: Brc1cnn2c(NCc3nc4ccccc4[nH]3)nc(N3CC4CCNC4C3)nc12]

Tert-butyl 5-[4-(1H-benzimidazol-2-ylmethylamino)-8-bromo-pyrazolo[1,5-a][1,3,5]triazin-2-yl]-2,3,3a,4,6,6a-hexahydropyrrolo[2,3-c]pyrrole-1-carboxylate (Intermediate 176, 153 mg, 90% purity, 248 μmol) was provided in dichloromethane (2.0 mL). Trifluoroacetic acid (190 μL, 2.5 mmol; CAS-RN:[76-05-1]) was added and the mixture was stirred for 17 h at rt. The reaction mixture was concentrated under vacuum, the residue was dissolved in methanol and triethylamine (200 μL) was added. The solution was concentrated under reduced pressure and purified by preparative HPLC (Method HT basic) to give 82.6 mg (70% yield) of the title compound.

LC-MS (Method 2): Rₜ=0.97 min; MS (ESIneg): m/z=452 [M−H]⁻

¹H-NMR (400 MHz, DMSO-d6) δ[ppm]: 1.234 (0.69), 1.754 (0.49), 1.779 (0.44), 1.900 (0.62), 2.325 (0.52), 2.329 (0.75), 2.334 (0.52), 2.521 (2.38), 2.525 (1.64), 2.542 (2.52), 2.667 (0.73), 2.671 (0.96), 2.676 (0.76), 2.681 (0.52), 2.729 (0.47), 2.793 (1.30), 3.512 (1.17), 3.729 (1.04), 4.876 (5.56), 7.109 (0.65), 7.119 (3.36), 7.127 (3.24), 7.134 (3.39), 7.142 (3.68), 7.152 (0.73), 7.478 (0.74), 7.995 (16.00), 8.005 (0.99), 8.339 (1.52).

### Example 272

### N-[(1H-benzimidazol-2-yl)methyl]-8-bromo-2-[4-(methylamino)piperidin-1-yl]pyrazolo[1,5-a][1,3,5]triazin-4-amine

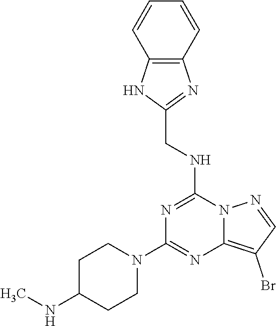 [SMILES: CNC1CCN(c2nc(NCc3nc4ccccc4[nH]3)n3ncc(Br)c3n2)CC1]

Tert-butyl [1-(4-{[(1H-benzimidazol-2-yl)methyl]amino}-8-bromopyrazolo[1,5-a][1,3,5]triazin-2-yl)piperidin-4-yl]methylcarbamate (Intermediate 177, 152 mg, 273 μmol) was provided in dichloromethane (2.0 mL). Trifluoroacetic acid (190 μL, 2.5 mmol; CAS-RN:[76-05-1]) was added and the mixture was stirred for 72 h at rt. The reaction mixture was concentrated under vacuum, the residue was dissolved in methanol and triethylamine (200 μL) was added. The solution was concentrated under reduced pressure and purified by preparative HPLC (Method HT basic) to give 75.4 mg (58% yield) of the title compound.

LC-MS (Method 2): Rₜ=1.00 min; MS (ESIpos): m/z=456 [M+H]⁺

¹H-NMR (400 MHz, DMSO-d6) δ[ppm]: 2.074 (0.99), 2.212 (16.00), 2.427 (0.58), 2.518 (2.54), 2.523 (1.61), 2.539 (1.53), 2.921 (0.45), 2.949 (0.77), 2.978 (0.46), 3.160 (1.51), 3.170 (1.56), 4.323 (0.48), 4.850 (5.03), 7.117 (1.31), 7.131 (1.41), 7.403 (0.45), 7.521 (0.48), 7.999 (9.60).

### Example 273

### N-[(5-fluoro-1H-benzimidazol-2-yl)methyl]-2-(morpholin-4-yl)-8-(trifluoromethyl)pyrazolo[1,5-a][1,3,5]triazin-4-amine

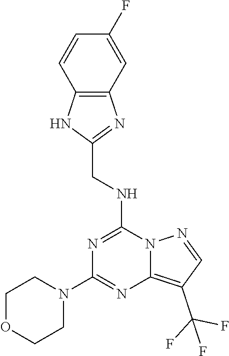 [SMILES: Fc1ccc2[nH]c(CNc3nc(N4CCOCC4)nc4c(C(F)(F)F)cnn34)nc2c1]

N-[(5-fluoro-1-{[2-(trimethylsilyl)ethoxy]methyl}-1H-benzimidazol-2-yl)methyl]-2-(morpholin-4-yl)-8-(trifluoromethyl)pyrazolo[1,5-a][1,3,5]triazin-4-amine (Intermediate 179, 170 mg, 300 μmol, 50% impure with N-[(6-fluoro-1-{[2-(trimethylsilyl)ethoxy]methyl}-1H-benzimidazol-2-yl)methyl]-2-(morpholin-4-yl)-8-(trifluoromethyl)pyrazolo[1,5-a][1,3,5]triazin-4-axdmine) was provided in dichloromethane (4.0 mL). Trifluoroacetic acid (3.0 mL, 39 mmol; CAS-RN:[76-05-1]) was added and the mixture was stirred for 16 h at rt. The reaction mixture was concentrated under vacuum, the residue was purified by flash chromatography (silica gel, dichloromethane/EtOAc/EtOH gradient) and dried to give 120 mg (88% yield) of the title compound.

LC-MS (Method 1): Rₜ=1.03 min; MS (ESIpos): m/z=437 [M+H]⁺

¹H-NMR (400 MHz, DMSO-d6) δ[ppm]: 1.154 (3.59), 1.172 (7.29), 1.190 (3.52), 1.232 (0.82), 1.987 (11.12), 2.127 (0.46), 2.318 (0.71), 2.337 (0.55), 2.518 (5.84), 2.523 (4.24), 2.729 (0.47), 2.888 (0.60), 3.496 (2.57), 4.000 (1.05), 4.017 (2.82), 4.035 (2.83), 4.053 (1.05), 4.942 (8.28), 4.956 (8.20), 7.096 (1.60), 7.102 (1.81), 7.121 (2.73), 7.124 (2.97), 7.143 (1.78), 7.149 (1.90), 7.383 (2.89), 7.390 (2.99), 7.407 (3.04), 7.412 (2.97), 7.547 (2.89), 7.559 (3.07), 7.569 (2.88), 7.581 (2.71), 8.275 (16.00), 9.339 (1.99), 9.354 (4.26), 9.368 (2.03).

### Example 274

### N-[(4-fluoro-1H-benzimidazol-2-yl)methyl]-2-(morpholin-4-yl)-8-(trifluoromethyl)pyrazolo[1,5-a][1,3,5]triazin-4-amine

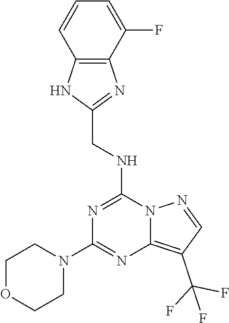 [SMILES: Fc1cccc2[nH]c(CNc3nc(N4CCOCC4)nc4c(C(F)(F)F)cnn34)nc12]

N-[(4-fluoro-1-{[2-(trimethylsilyl)ethoxy]methyl}-1H-benzimidazol-2-yl)methyl]-2-(morpholin-4-yl)-8-(trifluoromethyl)pyrazolo[1,5-a][1,3,5]triazin-4-amine (Intermediate 181, 175 mg, 310 μmol, 50% impure N-[(7-fluoro-1-{[2-(trimethylsilyl)ethoxy]methyl}-1H-benzimidazol-2-yl)methyl]-2-(morpholin-4-yl)-8-(trifluoromethyl)pyrazolo[1,5-a][1,3,5]triazin-4-amine) was provided in dichloromethane (4.0 mL). Trifluoroacetic acid (3.0 mL, 39 mmol; CAS-RN:[76-05-1]) was added and the mixture was stirred for 30 min at rt and for 30 min at 60° C. The reaction mixture was concentrated under vacuum, the residue was purified by flash chromatography (silica gel, dichloromethane/EtOAc) and dried to give 118 mg (81% yield) of the title compound.

LC-MS (Method 1): Rₜ=1.11 min; MS (ESIpos): m/z=437 [M+H]⁺

¹H-NMR (400 MHz, DMSO-d6) δ[ppm]: 1.154 (4.93), 1.172 (9.33), 1.190 (4.49), 1.232 (0.84), 1.988 (16.00), 2.336 (0.44), 2.518 (4.87), 2.523 (3.30), 2.678 (0.45), 3.494 (1.60), 4.000 (1.44), 4.017 (3.89), 4.035 (3.89), 4.053 (1.41), 4.909 (5.63), 4.924 (5.60), 6.972 (1.49), 6.993 (1.99), 7.000 (1.60), 7.020 (1.87), 7.133 (1.28), 7.146 (1.34), 7.154 (2.59), 7.166 (2.58), 7.174 (1.47), 7.186 (1.39), 7.293 (3.55), 7.312 (2.67), 8.267 (9.30), 9.324 (1.26), 9.338 (2.67), 9.352 (1.22).

### Example 275

### N-[(5-chloro-1H-benzimidazol-2-yl)methyl]-2-(morpholin-4-yl)-8-(trifluoromethyl)pyrazolo[1,5-a][1,3,5]triazin-4-amine

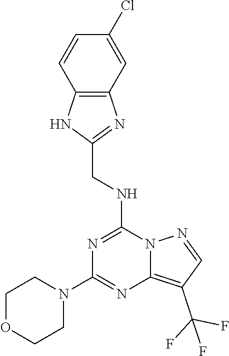 [SMILES: FC(F)(F)c1cnn2c(NCc3nc4cc(Cl)ccc4[nH]3)nc(N3CCOCC3)nc12]

N-[(5-chloro-1-{[2-(trimethylsilyl)ethoxy]methyl}-1H-benzimidazol-2-yl)methyl]-2-(morpholin-4-yl)-8-(trifluoromethyl)pyrazolo[1,5-a][1,3,5]triazin-4-amine (Intermediate 183, 145 mg, 250 μmol, 50% impure N-[(6-chloro-1-{[2-(trimethylsilyl)ethoxy]methyl}-1H-benzimidazol-2-yl)methyl]-2-(morpholin-4-yl)-8-(trifluoromethyl)pyrazolo[1,5-a][1,3,5]triazin-4-amine) was provided in dichloromethane (4.0 mL). Trifluoroacetic acid (3.0 mL, 39 mmol; CAS-RN:[76-05-1]) was added and the mixture was stirred for 16 h at rt. The reaction mixture was concentrated under vacuum, the residue was purified by flash chromatography (silica gel, dichloromethane/EtOAc) and dried to give 94.0 mg (81% yield) of the title compound.

LC-MS (Method 1): Rₜ=1.14 min; MS (ESIpos): m/z=453 [M+H]⁺

¹H-NMR (400 MHz, DMSO-d6) δ[ppm]: 0.752 (0.51), 0.771 (0.52), 0.851 (0.50), 1.154 (3.16), 1.172 (4.66), 1.190 (2.31), 1.232 (1.97), 1.988 (6.76), 2.336 (0.91), 2.518 (10.60), 2.523 (6.96), 2.678 (0.96), 2.729 (0.59), 2.888 (0.75), 3.488 (3.28), 4.000 (0.71), 4.017 (1.73), 4.035 (1.71), 4.053 (0.67), 4.906 (9.13), 4.920 (9.09), 7.207 (4.22), 7.211 (4.31), 7.228 (4.81), 7.233 (5.06), 7.510 (6.28), 7.532 (5.49), 7.584 (6.04), 7.589 (6.04), 8.269 (16.00), 9.321 (2.20), 9.335 (4.81), 9.349 (2.17).

### Example 276

### N-[(4,5-difluoro-1H-benzimidazol-2-yl)methyl]-2-(morpholin-4-yl)-8-(oxan-4-yl)pyrazolo[1,5-a][1,3,5]triazin-4-amine

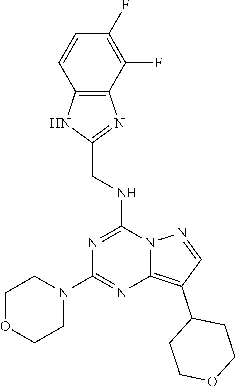 [SMILES: Fc1ccc2[nH]c(CNc3nc(N4CCOCC4)nc4c(C5CCOCC5)cnn34)nc2c1F]

To a solution of N-[(4,5-difluoro-1-{[2-(trimethylsilyl)ethoxy]methyl}-1H-benzimidazol-2-yl)methyl]-2-(morpholin-4-yl)-8-(oxan-4-yl)pyrazolo[1,5-a][1,3,5]triazin-4-amine (Intermediate 187, 150 mg, 250 μmol) in dichloromethane (3.0 mL) was added trifluoroacetic acid (1.5 mL, 19 mmol; CAS-RN:[76-05-1]) in one portion. The reaction mixture was stirred at rt for 16 h. The reaction mixture was concentrated and purified by preparative HPLC to give 58.0 mg (18.0 mg (95% purity, 15% yield) of the title compound as an off-white solid.

HPLC-Method: Instrument: Gilson-281; Column: Phenomenex Synergi C18 150*25*10 μm; eluent A: water (0.225% formic acid), eluent B: acetonitrile; gradient: 0-10 min 18-48% B; flow 25 mL/min; temperature: room temperature; Detector: UV 220/254 nm LC-MS (Method D): Rₜ=0.846 min; MS (ESIpos): m/z=471.4 [M+H]⁺.

¹H-NMR (400 MHz, DMSO-d₆) 5 [ppm]: 1.72-1.86 (m, 4H), 2.80-2.91 (m, 1H), 3.46-3.53 (m, 5H), 3.57 (br d, 4H), 3.87-3.95 (m, 2H), 4.87 (d, 2H), 7.23 (br s, 2H), 7.85 (s, 1H), 8.92-9.02 (m, 1H), 12.50-12.83 (m, 1H)

### Example 277

### N-[(4-methoxy-1H-benzimidazol-2-yl)methyl]-2-(morpholin-4-yl)-8-(2,2,2-trifluoroethyl)pyrazolo[1,5-a][1,3,5]triazin-4-amine

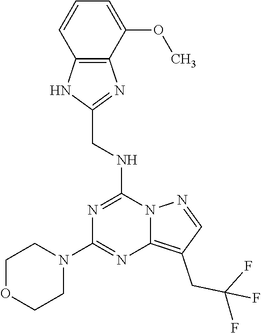 [SMILES: COc1cccc2[nH]c(CNc3nc(N4CCOCC4)nc4c(CC(F)(F)F)cnn34)nc12]

A solution of N-[(4-methoxyphenyl)methyl]-N-[[4-methoxy-1-(2-trimethylsilylethoxymethyl)benzimidazol-2-yl]methyl]-2-morpholino-8-(2,2,2-trifluoroethyl)pyrazolo[1,5-a][1,3,5]triazin-4-amine (Intermediate 190, 120 mg, 66% purity, 111 μmol) in trifluoroacetic acid (2.0 mL) was stirred at 80° C. for 16 h. The solution was diluted with water, and adjusted to pH=7 by sodium bicarbonate and extracted with ethyl acetate. The organic phase was washed with brine, dried over anhydrous sodium sulfate and filtered. The filtrate was concentrated and purified by prepative HPLC to give 19.2 mg (94% purity, 35% yield) of the title compound as a white solid.

HPLC-Method: Instrument:ACSWH-GX-G; Column:Phenomenex Synergi C18 150*25*10 um; eluent A: water (0.225% FA), eluent B: acetonitrile; gradient: 0-8 min 25-55% B; flow 25 mL/min; temperature: RT; Detector: UV 220/254 nm.

LC-MS (Method C): Rₜ=0.80 min; MS (ESIpos): m/z=463 [M+H]⁺

### Example 278

### N-[(4,5-difluoro-1H-benzimidazol-2-yl)methyl]-8-(2,2-difluorocyclopropyl)-2-(morpholin-4-yl)pyrazolo[1,5-a][1,3,5]triazin-4-amine

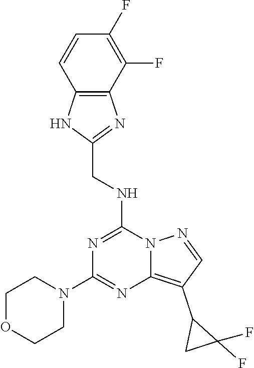 [SMILES: Fc1ccc2[nH]c(CNc3nc(N4CCOCC4)nc4c(C5CC5(F)F)cnn34)nc2c1F]

A solution of 8-(2,2-difluorocyclopropyl)-N-[(4,5-difluoro-1-{[2-(trimethylsilyl)ethoxy]methyl}-1H-benzimidazol-2-yl)methyl]-2-(morpholin-4-yl)pyrazolo[1,5-a][1,3,5]triazin-4-amine-8-(2,2-difluorocyclopropyl)-N-[(6,7-difluoro-1-{[2-(trimethylsilyl)ethoxy]methyl}-1H-benzimidazol-2-yl)methyl]-2-(morpholin-4-yl)pyrazolo[1,5-a][1,3,5]triazin-4-amine (1/1) (Intermediate 194, 200 mg, 45% purity, 152 μmol) in a mixed solvent of trifluoroacetic acid (4.0 mL, 52 mmol; CAS-RN:[76-05-1]) and dichloromethane (4.0 mL) was stirred at rt for 16 h. The reaction mixture was evaporated under reduced pressure to give a residue. The residue was purified by preparative HPLC to give 35.6 mg (99% purity, 50% yield) of the title compound as a light green solid.

HPLC-Method: Instrument: Gilson-281; Column: Phenomenex Synergi C18 150*25 mm*3 um; eluent A: 0.225% formic acid in water, eluent B: acetonitrile; gradient: 0-10 min 38-68% B; flow 25 mL/min; temperature: room temperature; Detector: UV 220/254 nm

LC-MS (Method C): Rₜ=0.815 min; MS (ESIpos): m/z=463.1 [M+H]⁺

¹H-NMR (400 MHz, DMSO-d6) δ[ppm]: 0.106 (0.54), 1.070 (1.56), 1.088 (3.18), 1.105 (1.69), 1.261 (0.41), 1.291 (0.49), 1.381 (1.36), 1.907 (0.50), 1.925 (1.06), 1.939 (1.64), 1.956 (1.86), 1.970 (1.95), 1.990 (1.80), 2.002 (1.90), 2.010 (1.83), 2.022 (2.25), 2.042 (2.02), 2.053 (1.50), 2.072 (0.61), 2.361 (0.43), 2.574 (2.60), 2.718 (1.73), 2.738 (1.84), 2.748 (3.00), 2.768 (2.91), 2.778 (1.79), 2.798 (1.69), 3.202 (1.41), 3.447 (3.09), 3.465 (4.52), 3.482 (5.14), 3.616 (16.00), 3.898 (1.25), 4.092 (0.64), 4.897 (8.18), 4.911 (8.37), 7.190 (1.04), 7.212 (2.20), 7.218 (1.59), 7.230 (2.36), 7.241 (2.37), 7.258 (2.72), 7.267 (3.80), 7.277 (4.30), 7.289 (2.24), 7.299 (1.86), 7.881 (11.00), 8.169 (0.99), 9.061 (2.08), 9.075 (4.15), 9.090 (2.12).

### Example 279

### N-[(1H-benzimidazol-2-yl)methyl]-8-(2,2-difluorocyclopropyl)-2-(morpholin-4-yl)pyrazolo[1,5-a][1,3,5]triazin-4-amine

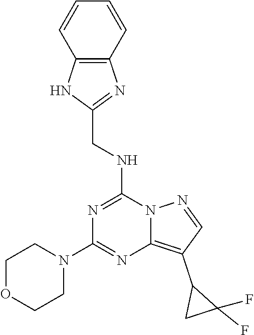 [SMILES: FC1(F)CC1c1cnn2c(NCc3nc4ccccc4[nH]3)nc(N3CCOCC3)nc12]

A solution of 8-(2,2-difluorocyclopropyl)-2-(morpholin-4-yl)-N-[(1-{[2-(trimethylsilyl)ethoxy]methyl}-1H-benzimidazol-2-yl)methyl]pyrazolo[1,5-a][1,3,5]triazin-4-amine (Intermediate 195, 180 mg, 51% purity, 165 μmol) in a mixed solvent of trifluoroacetic acid (2.0 mL, 26 mmol; CAS-RN:[76-05-1]) and dichloromethane (2.0 mL) was stirred at rt for 16 h. The reaction mixture was evaporated under reduced pressure to give a residue. The residue was purified by preparative HPLC to give 56.7 mg (99% purity, 80% yield) of the title compound as a white solid.

HPLC-Method: Instrument: Gilson-281; Column: Phenomenex Synergi C18 150*25 mm*3 um; eluent A: 0.225% formic acid in water, eluent B: acetonitrile; gradient: 0-10 min 18-48% B; flow 25 mL/min; temperature: room temperature; Detector: UV 220/254 nm

LC-MS (Method C): Rₜ=0.702 min; MS (ESIpos): m/z=427.2 [M+H]⁺

¹H-NMR (400 MHz, DMSO-d6) δ[ppm]: 0.106 (0.54), 1.070 (1.56), 1.088 (3.18), 1.105 (1.69), 1.261 (0.41), 1.291 (0.49), 1.381 (1.36), 1.907 (0.50), 1.925 (1.06), 1.939 (1.64), 1.956 (1.86), 1.970 (1.95), 1.990 (1.80), 2.002 (1.90), 2.010 (1.83), 2.022 (2.25), 2.042 (2.02), 2.053 (1.50), 2.072 (0.61), 2.361 (0.43), 2.574 (2.60), 2.718 (1.73), 2.738 (1.84), 2.748 (3.00), 2.768 (2.91), 2.778 (1.79), 2.798 (1.69), 3.202 (1.41), 3.447 (3.09), 3.465 (4.52), 3.482 (5.14), 3.616 (16.00), 3.898 (1.25), 4.092 (0.64), 4.897 (8.18), 4.911 (8.37), 7.190 (1.04), 7.212 (2.20), 7.218 (1.59), 7.230 (2.36), 7.241 (2.37), 7.258 (2.72), 7.267 (3.80), 7.277 (4.30), 7.289 (2.24), 7.299 (1.86), 7.881 (11.00), 8.169 (0.99), 9.061 (2.08), 9.075 (4.15), 9.090 (2.12).

In analogy to the procedures described above, the following examples were prepared using the appropriate intermediates as starting materials.

TABLE 2
Example 280-383

| | Structure | |
|---|---|---|
| | IUPAC-Name | Yield |
| Example | NMR | Analytics |
| 280 | 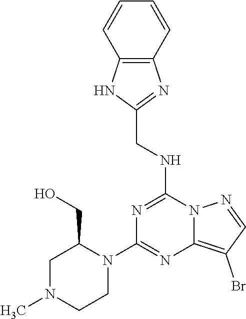 [SMILES: CN1CCN(c2nc(NCc3nc4ccccc4[nH]3)n3ncc(Br)c3n2)[C@H](CO)C1] | 12% LC-MS (Method 2) Rₜ = 0.98 min; MS (ESIpos): m/z = 472 [M + H]⁺ |
| | [(2R or S)-1-(4-{[(1H-benzimidazol-2-yl)methyl]amino}-8- | |
| | bromopyrazolo[1,5-a][1,3,5]triazin-2-yl)-4-methylpiperazin-2- | |
| | yl]methanol (Enantiomer 1) | |
| | ¹H-NMR (400 MHz, DMSO-d6) δ [ppm]: −0.008 (0.64), 0.000 (16.00), | |
| | 0.008 (0.59), 1.140 (2.49), 1.816 (1.03), 1.837 (1.03), 2.077 (0.54), | |
| | 2.087 (0.56), 2.119 (5.39), 2.329 (0.91), 2.334 (0.68), 2.521 (3.64), | |
| | 2.525 (2.27), 2.672 (0.96), 2.676 (0.71), 2.908 (0.64), 3.644 (0.51), | |
| | 4.675 (1.19), 4.682 (1.19), 4.866 (1.52), 7.108 (0.47), 7.122 (1.71), | |
| | 7.132 (2.35), 7.139 (2.37), 7.144 (2.54), 7.154 (1.80), 7.168 (0.47), | |
| | 7.413 (1.20), 7.431 (1.12), 7.534 (1.41), 7.552 (1.24), 8.009 (9.53), | |
| | 9.074 (1.17). | |
| 281 | 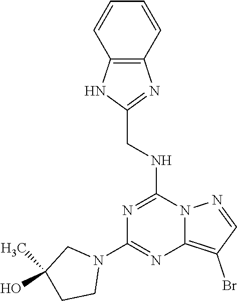 [SMILES: C[C@@]1(O)CCN(c2nc(NCc3nc4ccccc4[nH]3)n3ncc(Br)c3n2)C1] | 18% LC-MS (Method 2): Rₜ = 0.97 min; MS (ESIpos): m/z = 443 [M + H]⁺ |
| | (3R or S)-1-(4-{[(1H-benzimidazol-2-yl)methyl]amino}-8- | |
| | bromopyrazolo[1,5-a][1,3,5]triazin-2-yl)-3-methylpyrrolidin-3-ol | |
| | (Enantiomer 1) | |
| | ¹H-NMR (400 MHz, DMSO-d6) δ [ppm]: 0.721 (0.51), 0.798 (0.93), | |
| | 0.802 (0.56), 0.814 (1.07), 0.821 (1.11), 0.831 (0.67), 0.841 (1.09), | |
| | 0.847 (1.30), 0.850 (1.34), 0.857 (1.47), 0.869 (0.69), 0.873 (1.03), | |
| | 0.886 (0.66), 0.904 (1.18), 0.922 (0.59), 1.024 (0.87), 1.119 (1.40), | |
| | 1.134 (1.12), 1.229 (7.02), 1.292 (7.06), 1.727 (0.75), 1.750 (1.13), | |
| | 1.770 (1.48), 1.791 (1.02), 1.815 (0.90), 1.833 (0.71), 1.906 (0.60), | |
| | 2.332 (0.86), 2.336 (0.44), 2.518 (4.84), 2.522 (2.99), 2.539 (16.00), | |
| | 2.673 (0.89), 2.678 (0.43), 3.155 (0.96), 3.185 (1.19), 3.238 (1.13), | |
| | 3.268 (1.45), 3.392 (1.45), 3.422 (1.18), 3.457 (0.50), 3.494 (1.77), | |
| | 3.523 (1.50), 3.554 (0.58), 3.585 (0.66), 4.726 (2.98), 4.734 (3.10), | |
| | 4.879 (3.53), 7.097 (0.59), 7.112 (2.00), 7.122 (2.40), 7.128 (3.31), | |
| | 7.135 (2.57), 7.144 (2.22), 7.159 (0.67), 7.397 (2.19), 7.404 (1.40), | |
| | 7.414 (1.99), 7.418 (1.77), 7.529 (1.84), 7.546 (1.77), 7.984 (11.24), | |
| | 8.978 (0.73), 12.250 (2.62). | |
| 282 | 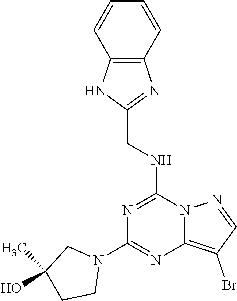 [SMILES: C[C@@]1(O)CCN(c2nc(NCc3nc4ccccc4[nH]3)n3ncc(Br)c3n2)C1] | 23% LC-MS (Method 2): Rₜ = 0.97 min; MS (ESIpos): m/z = 443 [M + H]⁺ |
| | (3R or S)-1-(4-{[(1H-benzimidazol-2-yl)methyl]amino}-8- | |
| | bromopyrazolo[1,5-a][1,3,5]triazin-2-yl)-3-methylpyrrolidin-3-ol | |
| | (Enantiomer 2) | |
| | ¹H-NMR (400 MHz, DMSO-d6) δ [ppm]: 0.787 (0.60), 0.814 (0.63), | |
| | 0.821 (0.70), 0.832 (0.51), 0.842 (0.79), 0.847 (1.06), 0.851 (1.06), | |
| | 0.858 (1.12), 0.869 (0.53), 0.874 (0.82), 0.904 (0.73) 1.024 (0.65), | |
| | 1.119 (0.81), 1.135 (0.89), 1.229 (5.28), 1.293 (5.35), 1.751 (0.84), | |
| | 1.770 (1.11), 1.781 (0.78), 1.793 (0.77), 1.814 (0.68), 2.332 (0.60), | |
| | 2.518 (3.53), 2.522 (2.18), 2.539 (16.00), 2.673 (0.62), 3.156 (0.72), | |
| | 3.185 (0.89), 3.239 (0.83), 3.268 (1.02), 3.393 (0.99), 3.422 (0.88), | |
| | 3.494 (1.34), 3.523 (1.14), 3.577 (0.43), 3.565 (0.42), 3.586 (0.49), | |
| | 4.724 (2.34), 4.732 (2.41), 4.880 (2.64), 7.097 (0.45), 7.112 (1.50), | |
| | 7.122 (1.80), 7.128 (2.58), 7.135 (1.94), 7.144 (1.71), 7.159 (0.53), | |
| | 7.397 (1.70), 7.404 (1.08), 7.414 (1.55), 7.419 (1.37), 7.530 (1.41), | |
| | 7.547 (1.37), 7.985 (8.02), 8.977 (0.58), 12.250 (2.07). | |
| 283 | 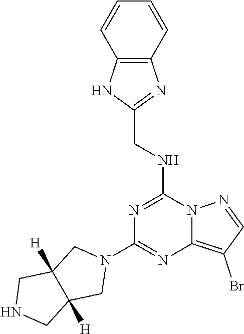 [SMILES: Brc1cnn2c(NCc3nc4ccccc4[nH]3)nc(N3C[C@H]4CNC[C@H]4C3)nc12] | 31% LC-MS (Method 2): Rₜ = 0.93 min; MS (ESIneg): m/z = 452 [M − H]⁻ |
| | N-[(1H-benzimidazol-2-yl)methyl]-8-bromo-2-[(3aR,6aS)- | |
| | hexahydropyrrolo[3,4-c]pyrrol-2(1H)-yl]pyrazolo[1,5- | |
| | a][1,3,5]triazin-4-amine | |
| | ¹H-NMR (400 MHz, DMSO-d6) δ [ppm]: −0.003 (1.67), 1.231 (0.83), | |
| | 1.771 (0.45), 1.875 (1.75), 2.323 (0.89), 2.327 (1.20), 2.331 (0.88), | |
| | 2.518 (6.12), 2.523 (4.45), 2.540 (4.13), 2.665 (1.59), 2.669 (2.01), | |
| | 2.673 (1.76), 2.829 (1.51), 3.636 (1.18), 4.869 (7.31), 7.119 (3.21), | |
| | 7.126 (3.35), 7.134 (3.45), 7.141 (3.45), 7.426 (0.72), 7.514 (0.70), | |
| | 7.992 (16.00), 8.001 (0.65). | |
| 284 | 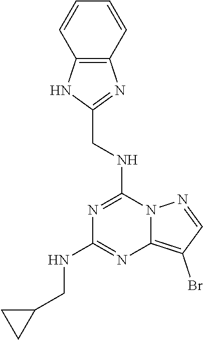 [SMILES: Brc1cnn2c(NCc3nc4ccccc4[nH]3)nc(NCC3CC3)nc12] | 56% LC-MS (Method 2): Rₜ = 1.10 min; MS (ESIpos): m/z 413 [M + H]⁺ |
| | N⁴-[(1H-benzimidazol-2-yl)methyl]-8-bromo-N²- | |
| | (cyclopropylmethyl)pyrazolo[1,5-a][1,3,5]triazine-2,4-diamine | |
| | ¹H-NMR (400 MHz, DMSO-d6) δ [ppm]: −0.046 (0.63), −0.003 (0.62), | |
| | 0.083 (0.53), 0.208 (1.56), 0.219 (1.63), 0.388 (1.28), 0.406 (1.32), | |
| | 1.044 (0.48), 1.232 (0.46), 2.518 (1.70), 2.523 (1.08), 2.968 (0.58), | |
| | 3.102 (1.02), 3.118 (1.64), 3.133 (0.95), 4.857 (3.25), 4.869 (3.15), | |
| | 7.127 (1.80), 7.380 (1.20), 7.395 (1.16), 7.531 (0.87), 7.974 (16.00), | |
| | 8.879 (0.60), 12.241 (1.21). | |
| 285 | 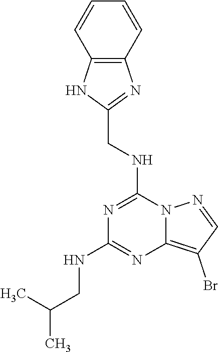 [SMILES: CC(C)CNc1nc(NCc2nc3ccccc3[nH]2)n2ncc(Br)c2n1] | 52% LC-MS (Method 2): Rₜ = 1.15 min; MS (ESIpos): m/z = 415 [M + H]⁺ |
| | N⁴-[(1H-benzimidazol-2-yl)methyl]-8-bromo-N²-(2- | |
| | methylpropyl)pyrazolo[1,5-a][1,3,5]triazine-2,4-diamine | |
| | ¹H-NMR (400 MHz, DMSO-d6) δ [ppm]: 0.563 (4.03), 0.579 (4.17), | |
| | 0.866 (8.10), 0.875 (4.17), 0.882 (8.28), 1.232 (1.19), 1.473 (0.41), | |
| | 1.851 (0.59), 1.867 (0.70), 1.883 (0.55), 2.518 (3.24), 2.522 (2.04), | |
| | 2.539 (0.66), 2.859 (0.73), 2.874 (1.23), 3.065 (1.40), 3.081 (2.26), | |
| | 3.097 (1.34), 4.860 (2.93), 4.875 (2.24), 7.133 (2.45), 7.307 (1.13), | |
| | 7.370 (0.68), 7.398 (1.35), 7.415 (1.03), 7.463 (0.67), 7.531 (1.47), | |
| | 7.550 (0.97), 7.884 (0.45), 7.969 (16.00), 8.857 (1.03), 9.024 (0.62), | |
| | 12.229 (3.41). | |
| 286 | 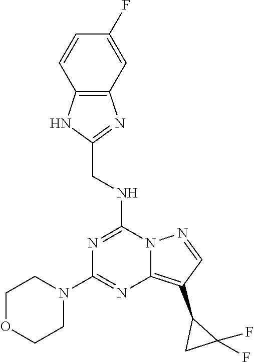 [SMILES: Fc1ccc2[nH]c(CNc3nc(N4CCOCC4)nc4c([C@@H]5CC5(F)F)cnn34)nc2c1] | 71% LC-MS (Method 2): Rₜ = 1.09 min; MS (ESIpos): m/z = 445 [M + H]⁺ |
| | 8-[(1RS)-2,2-difluorocyclopropyl]-N-[(5-fluoro-1H-benzimidazol-2- | |
| | yl)methyl]-2-(morpholin-4-yl)pyrazolo[1,5-a][1,3,5]triazin-4-amine | |
| | ¹H-NMR (400 MHz, DMSO-d6) δ [ppm]: 1.035 (3.60), 1.052 (7.20), | |
| | 1.070 (3.87), 1.231 (0.97), 1.306 (0.48), 1.881 (0.58), 1.900 (1.09), | |
| | 1.913 (1.56), 1.929 (1.56), 1.944 (1.76), 1.966 (1.24), 1.976 (1.54), | |
| | 1.986 (1.71), 1.998 (2.00), 2.018 (1.83), 2.029 (1.33), 2.038 (0.72), | |
| | 2.049 (0.48), 2.518 (16.00), 2.522 (11.32), 2.687 (1.61), 2.707 (1.53), | |
| | 2.718 (3.03), 2.729 (0.96), 2.737 (2.90), 2.748 (1.52), 2.767 (1.30), | |
| | 2.888 (0.82), 3.410 (1.84), 3.428 (4.45), 3.445 (5.69), 3.463 (6.86), | |
| | 3.473 (6.96), 3.901 (0.60), 4.956 (6.41), 4.970 (6.48), 7.166 (1.39), | |
| | 7.188 (2.38), 7.212 (1.29), 7.447 (2.27), 7.464 (2.27), 7.595 (2.39), | |
| | 7.607 (2.37), 7.617 (2.20), 7.628 (1.98), 7.717 (0.82), 7.873 (12.47), | |
| | 9.106 (1.65), 9.121 (3.37), 9.134 (1.161). | |
| 287 | 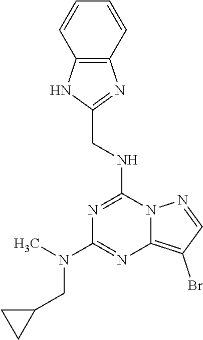 [SMILES: CN(CC1CC1)c1nc(NCc2nc3ccccc3[nH]2)n2ncc(Br)c2n1] | 89% LC-MS (Method 2): Rₜ = 1.25 min; MS (ESIpos): m/z = 427 [M + H]⁺ |
| | N⁴-[(1H-benzimidazol-2-yl)methyl]-8-bromo-N²- | |
| | (cyclopropylmethyl)-N²-methylpyrazolo[1,5-a][1,3,5]triazine-2,4- | |
| | diamine | |
| | ¹H-NMR (400 MHz, DMSO-d6) δ [ppm]: −0.029 (2.07), −0.003 (1.35), | |
| | 0.60 (1.92), 2.81 (1.08), 0.431 (0.91), 0.721 (0.75), 0.944 (0.92), | |
| | 0.960 (0.91), 1.108 (0.45), 2.074 (1.58), 2.522 (5.72), 3.063 (6.86), | |
| | 3.143 (0.53), 3.429 (1.21), 4.839 (3.29), 7.122 (4.68), 7.386 (1.99), | |
| | 7.517 (2.68), 7.533 (2.45), 7.913 (0.53), 7.999 (16.00), 9.010 (1.74), | |
| | 12.250 (2.10). | |
| 288 | 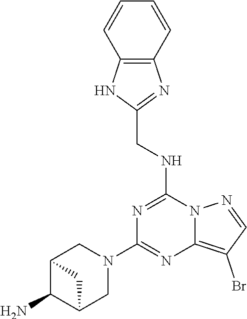 [SMILES: N[C@H]1[C@@H]2C[C@H]1CN(c1nc(NCc3nc4ccccc4[nH]3)n3ncc(Br)c3n1)C2] | 53% LC-MS (Method 2): Rₜ = 0.97 min; MS (ESIneg): m/z = 452 [M − H]⁻ |
| | 2-[(1R,5S,6r)-6-amino-3-azabicyclo[3.1.1]heptan-3-yl]-N-[(1H- | |
| | benzimidazol-2-yl)methyl]-8-bromopyrazolo[1,5-a][1,3,5]triazin- | |
| | 4-amine | |
| | ¹H-NMR (400 MHz, DMSO-d6) δ [ppm]: 1.035 (0.43), 1.052 (0.88), | |
| | 1.070 (0.46), 1.231 (2.71), 1.244 (2.02), 1.254 (1.94), 1.267 (1.77), | |
| | 2.035 (1.97), 2.050 (1.28), 2.115 (1.23), 2.130 (2.03), 2.522 (2.55), | |
| | 2.539 (2.05), 2.580 (0.74), 2.596 (1.42), 2.617 (1.38), 2.632 (0.64), | |
| | 2.936 (3.70), 2.949 (3.61), 3.165 (2.13), 3.445 (0.77), 3.490 (1.92), | |
| | 3.520 (2.31), 3.584 (1.80), 3.615 (2.23), 3.690 (2.09), 3.697 (2.20), | |
| | 3.720 (1.70), 3.727 (1.64), 3.785 (2.05), 3.792 (2.18), 3.815 (1.69), | |
| | 3.823 (1.55), 4.897 (12.38), 7.121 (3.32), 7.133 (3.49), 7.406 (1.06), | |
| | 7.525 (1.09), 8.012 (16.00). | |
| 289 | 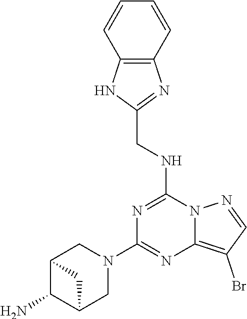 [SMILES: N[C@@H]1[C@@H]2C[C@H]1CN(c1nc(NCc3nc4ccccc4[nH]3)n3ncc(Br)c3n1)C2] | 45% LC-MS (Method 2): Rₜ = 1.02 min; MS (ESIpos): m/z = 454 [M + H]⁺ |
| | 2-[(1R,5S,6s)-6-amino-3-azabicyclo[3.1.1]heptan-3-yl]-N-[(1H- | |
| | benzimidazol-2-yl)methyl]-8-bromopyrazolo[1,5-a][1,3,5]triazin- | |
| | 4-amine | |
| | ¹H-NMR (400 MHz, DMSO-d6) δ [ppm]: 1.147 (3.26), 1.171 (3.49), | |
| | 1.231 (0.47), 1.622 (0.72), 1.638 (1.49), 1.651 (1.25), 1.660 (1.43), | |
| | 1.676 (0.74), 2.074 (3.37), 2.291 (1.51), 2.297 (1.50), 2.305 (1.68), | |
| | 2.322 (1.14), 2.326 (1.17), 2.331 (0.76), 2.383 (1.68), 2.390 (1.53), | |
| | 2.397 (1.53), 2.522 (2.59), 2.539 (0.61), 2.664 (0.62), 2.668 (0.82), | |
| | 2.673 (0.62), 3.166 (0.44), 3.196 (2.18), 3.211 (3.89), 3.225 (2.17), | |
| | 3.545 (1.02), 3.575 (2.93), 3.598 (2.82), 3.605 (3.36), 3.629 (1.31), | |
| | 3.642 (2.90), 3.687 (2.45), 3.694 (2.60), 3.719 (1.25), 3.725 (1.19), | |
| | 4.915 (9.60), 7.122 (3.32), 7.136 (3.61), 7.411 (1.19), 7.530 (1.25), | |
| | 8.013 (16.00), 12.210 (0.65). | |
| 290 | 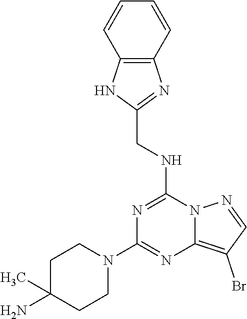 [SMILES: CC1(N)CCN(c2nc(NCc3nc4ccccc4[nH]3)n3ncc(Br)c3n2)CC1] | 57% LC-MS (Method 2): Rₜ = 1.00 min; MS (ESIpos): m/z = 456 [M + H]⁺ |
| | 2-(4-amino-4-methylpiperidin-1-yl)-N-[(1H-benzimidazol-2- | |
| | yl)methyl]-8-bromopyrazolo[1,5-a][1,3,5]triazin-4-amine | |
| | ¹H-NMR (400 MHz, DMSO-d6) δ [ppm]: 0.965 (16.00), 1.231 (1.00), | |
| | 2.522 (2.57), 2.539 (0.99), 3.165 (1.35), 3.464 (1.05), 3.881 (0.78), | |
| | 4.845 (9.60), 7.101 (0.65), 7.112 (3.53), 7.119 (3.43), 7.126 (3.57), | |
| | 7.134 (3.99), 7.144 (0.80), 7.460 (0.93), 7.991 (13.15). | |
| 291 | 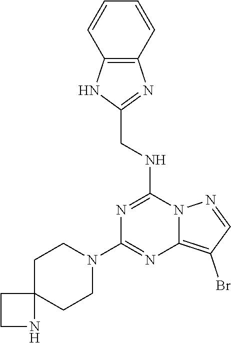 [SMILES: Brc1cnn2c(NCc3nc4ccccc4[nH]3)nc(N3CCC4(CCN4)CC3)nc12] | 35% LC-MS (Method 2): Rₜ = 1.05 min; MS (ESIpos): m/z = 468 [M + H]⁺ |
| | N-[(1H-benzimidazol-2-yl)methyl]-8-bromo-2-(1,7- | |
| | diazaspiro[3.5]nonan-7-yl)pyrazolo[1,5-a][1,3,5]triazin-4-amine | |
| | ¹H-NMR (400 MHz, DMSO-d6) δ[ppm]: 1.230 (0.49), 1.458 (0.89), | |
| | 1.898 (1.44), 1.916 (2.60), 1.935 (1.57), 2.518 (4.90), 2.523 (3.42), | |
| | 2.540 (9.10), 3.258 (2.24), 3.276 (4.00), 3.295 (2.85), 3.502 (1.16), | |
| | 3.664 (1.21), 4.851 (10.36), 7.118 (3.36), 7.125 (3.39), 7.133 (3.55), | |
| | 7.141 (3.66), 7.505 (0.62), 8.001 (16.00), 8.011 (0.89). | |
| 292 | 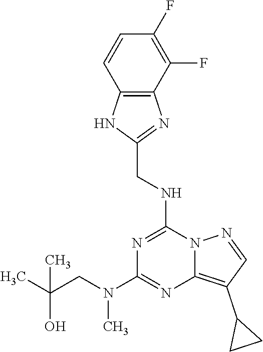 [SMILES: CN(CC(C)(C)O)c1nc(NCc2nc3c(F)c(F)ccc3[nH]2)n2ncc(C3CC3)c2n1] | 11% LC-MS (Method 1): Rₜ = 1.07 min; MS (ESIpos): m/z = 443 [M + H]⁺ |
| | 1-[(8-cyclopropyl-4-{[(4,5-difluoro-1H-benzimidazol-2- | |
| | yl)methyl]amino}pyrazolo[1,5-a][1,3,5]triazin-2- | |
| | yl)(methyl)amino]-2-methylpropan-2-ol | |
| | ¹H-NMR (400 MHz, DMSO-d6) δ [ppm]: 0.710 (12.50), 7.38 (11.28), | |
| | 0.744 (16.00), 0.749 (11.48), 0.751 (11.50), 0.757 (15.59), 0.762 | |
| | (13.96), 0.786 (8.41), 1.079 (3.00), 1.230 (1.12), 1.736 (1.83), 2.332 | |
| | (1.96), 2.336 (0.90), 2.518 (10.31), 2.522 (6.52), 2.539 (1.89), 2.673 | |
| | (2.00), 2.678 (0.90), 3.084 (8.27), 3.293 (1.46), 3.381 (3.37), 3.384 | |
| | (3.51), 3.397 (1.11), 3.427 (0.54), 3.504 (0.97), 4.214 (0.41), 4.285 | |
| | (2.23), 4.783 (3.90), 4.860 (1.10), 5.040 (0.53), 7.171 (4.87), 7.181 | |
| | (5.02), 7.323 (0.42), 7.681 (5.76), 8.795 (1.76), 12.635 (3.08), 13.100 | |
| | (0.87). | |
| 293 | 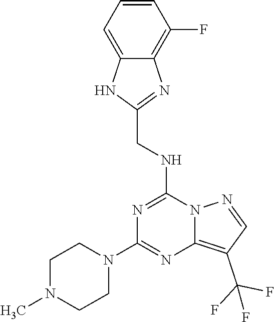 [SMILES: CN1CCN(c2nc(NCc3nc4c(F)cccc4[nH]3)n3ncc(C(F)(F)F)c3n2)CC1] | 58% LC-MS (Method 1): Rₜ = 0.78 min; MS (ESIneg): m/z = 448 [M − H]⁻ |
| | N-[(4-fluoro-1H-benzimidazol-2-yl)methyl]-2-(4-methylpiperazin- | |
| | 1-yl)-8-(trifluoromethyl)pyrazolo[1,5-a][1,3,5]triazin-4-amine | |
| | ¹H-NMR (400 MHz, DMSO-d6) δ [ppm]: −0.169 (0.42), 0.851 (0.42), | |
| | 1.232 (1.92), 1.256 (0.45), 1.549 (0.40), 2.109 (16.00), 2.231 (0.65), | |
| | 2.318 (0.57), 2.322 (1.08), 2.327 (1.42), 2.332 (1.05), 2.336 (0.50), | |
| | 2.518 (5.50), 2.522 (3.43), 2.660 (0.45), 2.664 (0.99), 2.669 (1.36), | |
| | 2.673 (0.99), 2.678 (0.44), 3.627 (1.59), 4.883 (5.68), 5.759 (5.99), | |
| | 6.946 (0.70), 6.972 (0.55), 7.098 (0.93), 7.111 (1.04), 7.119 (1.90), | |
| | 7.131 (1.93), 7.139 (1.10), 7.151 (1.06), 7.238 (0.64), 8.243 (6.92), | |
| | 9.281 (0.51). | |
| 294 | 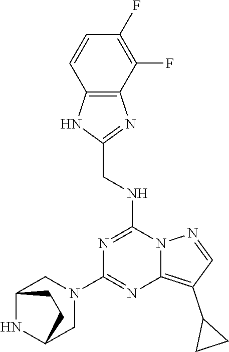 [SMILES: Fc1ccc2[nH]c(CNc3nc(N4C[C@H]5CC[C@@H](C4)N5)nc4c(C5CC5)cnn34)nc2c1F] | 19% LC-MS (Method 2): Rₜ = 1.04 min; MS (ESIpos): m/z = 452 [M + H]⁺ |
| | 8-cyclopropyl-2-[(1R,5S)-3,8-diazabicyclo[3.2.1]octan-3-yl]-N- | |
| | [(4,5-difluoro-1H-benzimidazol-2-yl)methyl]pyrazolo[1,5- | |
| | a][1,3,5]triazin-4-amine | |
| | ¹H-NMR (400 MHz, DMSO-d6) δ [ppm]: 0.720 (0.56), 0.727 (0.57), | |
| | 0.733 (0.55), 0.774 (0.51), 0.795 (0.50), 1.103 (0.49), 2.522 (1.38), | |
| | 2.539 (16.00), 4.815 (0.47), 7.723 (0.89). | |
| 295 | 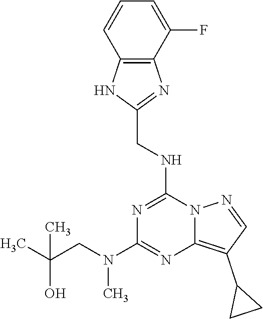 [SMILES: CN(CC(C)(C)O)c1nc(NCc2nc3c(F)cccc3[nH]2)n2ncc(C3CC3)c2n1] | 7% LC-MS (Method 2): Rₜ = 1.14 min; MS (ESIpos): m/z = 425 [M + H]⁺ |
| | 1-[(8-cyclopropyl-4-{[(4-fluoro-1H-benzimidazol-2- | |
| | yl)methyl]amino}pyrazolo[1,5-a][1,3,5]triazin-2- | |
| | yl)(methyl)amino]-2-methylpropan-2-ol | |
| | ¹H-NMR (400 MHz, DMSO-d6) δ [ppm]: 0.726 (10.54), 0.740 (12.95), | |
| | 0.746 (16.00), 0.751 (11.37), 0.754 (11.27), 0.759 (15.11), 0.764 | |
| | (13.20), 0.787 (7.54), 1.082 (2.72), 1.178 (1.72), 1.232 (0.86), 1.750 | |
| | (2.70), 2.522 (11.42), 2.539 (7.43), 3.087 (7.23), 3.379 (4.55), 3.507 | |
| | (1.00), 4.280 (0.91), 4.791 (3.16), 5.759 (7.23), 6.928 (1.83), 7.076 | |
| | (1.39), 7.096 (2.79), 7.108 (2.87), 7.127 (1.48), 7.226 (1.45), 7.679 | |
| | (4.63), 8.772 (1.22), 12.539 (1.31). | |
| 296 | 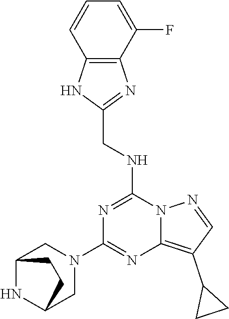 [SMILES: Fc1cccc2[nH]c(CNc3nc(N4C[C@H]5CC[C@@H](C4)N5)nc4c(C5CC5)cnn34)nc12] | 15% LC-MS (Method 2): Rₜ = 1.02 min; MS (ESIpos): m/z = 434 [M + H]⁺ |
| | 8-cyclopropyl-2-[(1R,5S)-3,8-diazabicyclo[3.2.1]octan-3-yl]-N-[(4- | |
| | fluoro-1H-benzimidazol-2-yl)methyl]pyrazolo[1,5-a][1,3,5]triazin- | |
| | 4-amine | |
| | ¹H-NMR (400 MHz, DMSO-d6) δ [ppm]: 0.694 (2.63), 0.712 (10.25), | |
| | 0.719 (11.22), 0.724 (9.83), 0.732 (4.68), 0.750 (5.72), 0.758 (9.38), | |
| | 0.762 (6.93), 0.771 (5.91), 0.779 (10.02), 0.795 (2.35), 0.851 (0.43), | |
| | 1.035 (0.74), 1.052 (1.06), 1.070 (0.94), 1.150 (1.12), 1.166 (1.12), | |
| | 1.233 (2.23), 1.349 (2.18), 1.401 (2.27), 1.650 (0.71), 1.680 (1.65), | |
| | 1.693 (2.93), 1.701 (3.16), 1.714 (4.60), 1.727 (2.92), 1.734 (2.63), | |
| | 1.750 (2.22), 2.288 (0.63), 2.336 (1.40), 2.518 (16.00), 2.522 (9.93), | |
| | 2.539 (7.71), 2.673 (3.35), 2.801 (3.22), 3.590 (1.46), 3.681 (0.43), | |
| | 3.701 (0.42), 4.104 (2.11), 4.643 (0.52), 4.799 (9.79), 5.758 (3.76), | |
| | 6.926 (2.37), 7.080 (1.84), 7.099 (3.77), 7.110 (3.69), 7.131 (1.82), | |
| | 7.230 (2.11), 7.589 (0.50), 7.673 (15.62), 8.831 (1.78), 12.547 (1.08). | |
| 297 | 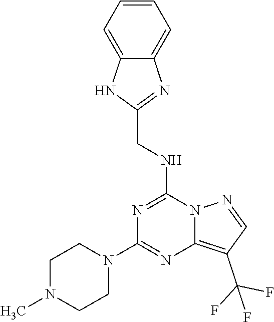 [SMILES: CN1CCN(c2nc(NCc3nc4ccccc4[nH]3)n3ncc(C(F)(F)F)c3n2)CC1] | 58% LC-MS (Method 1): Rₜ = 0.63 min; MS(ESIneg): m/z = 430 [M − H]⁻ |
| | N-[(1H-benzimidazol-2-yl)methyl]-2-(4-methylpiperazin-1-yl)-8- | |
| | (trifluoromethyl)pyrazolo[1,5-a][1,3,5]triazin-4-amine | |
| | ¹H-NMR (400 MHz, DMSO-d6) δ [ppm]: 0.884 (0.67), 0.901 (1.35), | |
| | 0.904 (0.50), 0.920 (0.75), 0.991 (0.50), 1.009 (0.49), 1.016 (0.41), | |
| | 1.035 (1.09), 1.052 (1.29), 1.070 (0.82), 1.232 (0.53), 1.987 (0.43), | |
| | 2.065 (2.01), 2.092 (0.89), 2.114 (16.00), 2.124 (2.36), 2.210 (0.69), | |
| | 2.318 (0.45), 2.322 (0.82), 2.326 (1.03), 2.332 (0.75), 2.518 (3.37), | |
| | 2.522 (2.20), 2.664 (0.69), 2.668 (0.92), 2.673 (0.68), 3.637 (1.70), | |
| | 4.869 (3.68), 4.883 (3.72), 5.759 (7.62), 7.098 (0.41), 7.102 (0.69), | |
| | 7.116 (2.24), 7.120 (1.90), 7.126 (2.44), 7.133 (3.84), 7.139 (2.68), | |
| | 7.145 (1.95), 7.149 (2.43), 7.163 (0.79), 7.390 (1.83), 7.397 (1.28), | |
| | 7.407 (1.85), 7.411 (1.48), 7.528 (1.62), 7.533 (1.71), 7.543 (0.96), | |
| | 7.550 (1.54), 8.240 (6.85), 9.229 (0.80), 9.244 (1.66), 9.259 (0.76), | |
| | 12.245 (2.31). | |
| 298 | 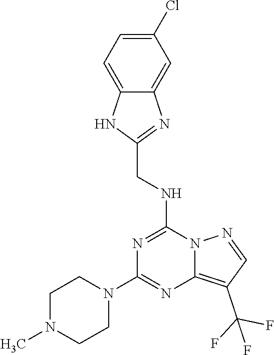 [SMILES: CN1CCN(c2nc(NCc3nc4cc(Cl)ccc4[nH]3)n3ncc(C(F)(F)F)c3n2)CC1] | 47% LC-MS (Method 1): Rₜ = 0.82 min; MS (ESIneg): m/z = 464 [M − H]⁻ |
| | N-[(5-chloro-1H-benzimidazol-2-yl)methyl]-2-(4-methylpiperazin- | |
| | 1-yl)-8-(trifluoromethyl)pyrazolo[1,5-a][1,3,5]triazin-4-amine | |
| | ¹H-NMR (400 MHz, DMSO-d6) δ [ppm]: 0.798 (1.91), 0.803 (0.85), | |
| | 0.815 (2.09), 0.822 (2.14), 0.840 (1.08), 0.886 (1.14), 0.905 (2.25), | |
| | 0.922 (1.19), 1.035 (2.63), 1.052 (4.72), 1.070 (2.86), 1.232 (1.42), | |
| | 1.256 (0.54), 2.114 (16.00), 2.170 (0.75), 2.190 (0.70), 2.210 (0.88), | |
| | 2.230 (0.83), 2.318 (0.62), 2.322 (1.19), 2.327 (1.63), 2.332 (1.19), | |
| | 2.336 (0.57), 2.413 (0.49), 2.518 (6.50), 2.523 (4.13), 2.660 (0.46), | |
| | 2.664 (1.06), 2.669 (1.52), 2.673 (1.11), 2.678 (0.49), 3.422 (0.75), | |
| | 3.435 (0.75), 3.439 (0.72), 3.452 (0.75), 3.625 (1.26), 4.343 (0.41), | |
| | 4.355 (0.77), 4.868 (3.41), 7.152 (0.88), 7.174 (0.95), 7.451 (0.41), | |
| | 7.600 (0.41), 8.241 (6.76), 9.265 (0.72), 12.459 (0.41). | |
| 299 | 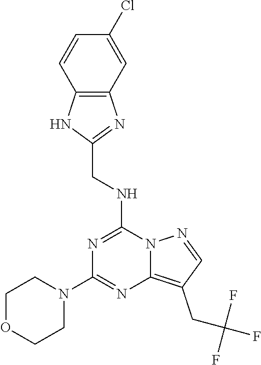 [SMILES: FC(F)(F)Cc1cnn2c(NCc3nc4cc(Cl)ccc4[nH]3)nc(N3CCOCC3)nc12] | 39% LC-MS (Method 1): Rₜ = 1.13 min; MS (ESIpos): m/z = 467 [M + H]⁺ |
| | N-[(5-chloro-1H-benzimidazol-2-yl)methyl]-2-(morpholin-4-yl)-8- | |
| | (2,2,2-trifluoroethyl)pyrazolo[1,5-a][1,3,5]triazin-4-amine | |
| | ¹H-NMR (400 MHz, DMSO-d6) δ [ppm]: 2.322 (0.74), 2.326 (1.05), | |
| | 2.522 (6.19), 2.539 (16.00), 2.664 (0.81), 2.668 (1.09), 2.673 (0.81), | |
| | 3.424 (2.53), 3.453 (5.95), 3.481 (10.40), 3.509 (2.75), 3.592 (7.11), | |
| | 3.729 (0.60), 4.854 (5.46), 4.868 (5.53), 7.148 (1.54), 7.169 (1.72), | |
| | 7.422 (1.00), 7.444 (1.05), 7.493 (0.68), 7.532 (0.63), 7.596 (0.88), | |
| | 7.911 (10.04), 9.064 (1.30), 9.078 (2.60), 9.093 (1.30), 12.494 (0.72). | |
| 300 | 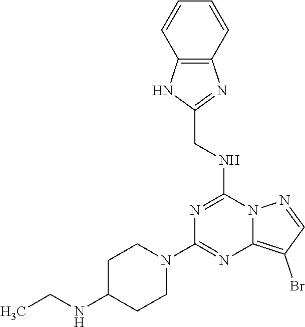 [SMILES: CCNC1CCN(c2nc(NCc3nc4ccccc4[nH]3)n3ncc(Br)c3n2)CC1] | 48% LC-MS (Method 2): Rₜ = 1.09 min; MS (ESIpos): m/z = 470 [M + H]⁺ |
| | N-[1H-benzimidazol-2-yl)methyl]-8-bromo-2-[4- | |
| | (ethylamino)piperidin-1-yl]pyrazolo[1,5-a][1,3,5]triazin-4-amine | |
| | ¹H-NMR (400 MHz, DMSO-d6) δ [ppm]: 0.949 (7.54), 0.967 (16.00), | |
| | 0.984 (8.13), 1.232 (0.52), 1.688 (0.95), 1.751 (0.59), 2.327 (1.00), | |
| | 2.331 (0.77), 2.467 (2.42), 2.539 (1.99), 2.549 (1.97), 2.573 (0.88), | |
| | 2.669 (1.08), 2.888 (1.47), 2.921 (2.20), 2.948 (1.29), 4.370 (1.36), | |
| | 4.851 (11.56), 7.118 (3.46), 7.129 (3.69), 7.404 (1.13), 7.522 (1.16), | |
| | 7.999 (14.14), 12.259 (1.09). | |
| 301 | 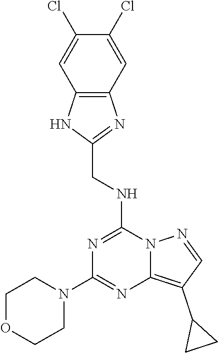 [SMILES: Clc1cc2nc(CNc3nc(N4CCOCC4)nc4c(C5CC5)cnn34)[nH]c2cc1Cl] | 19% LC-MS (Method 1): Rₜ = 1.21 min; MS (ESIneg): m/z = 457 [M − H]⁻ |
| | 8-cyclopropyl-N-[(5,6-dichloro-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: 0.704 (1.39), 0.708 (0.91), | |
| | 0.716 (4.52), 0.721 (5.57), 0.729 (6.65), 0.734 (6.17), 0.742 (2.67), | |
| | 0.753 (0.88), 0.760 (1.19), 0.766 (2.73), 0.774 (5.17), 0.779 (3.55), | |
| | 0.787 (3.81), 0.791 (2.25), 0.795 (5.40), 0.800 (3.41), 0.806 (1.05), | |
| | 0.812 (1.62), 1.372 (0.74), 1.704 (0.88), 1.716 (1.68), 1.725 (1.68), | |
| | 1.737 (2.70), 1.746 (0.99), 1.750 (1.56), 1.758 (1.53), 1.771 (0.74), | |
| | 2.323 (1.22), 2.327 (1.71), 2.332 (1.19), 2.518 (6.11), 2.523 (3.98), | |
| | 2.540 (1.96), 2.660 (0.54), 2.665 (1.25), 2.669 (1.73), 2.674 (1.19), | |
| | 2.679 (0.54), 3.485 (6.28), 3.496 (5.54), 3.540 (6.45), 3.552 (7.05), | |
| | 4.832 (5.60), 4.846 (5.54), 7.712 (16.00), 8.890 (1.31), 8.905 (2.79), | |
| | 8.919 (1.28), 12.583 (1.56). | |
| 302 | 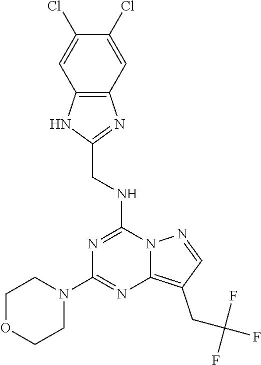 [SMILES: FC(F)(F)Cc1cnn2c(NCc3nc4cc(Cl)c(Cl)cc4[nH]3)nc(N3CCOCC3)nc12] | 25% LC-MS (Method 1): Rₜ = 1.26 min; MS (ESIpos): m/z = 501 [M + H]⁺ |
| | N-[(5,6-dichloro-1H-benzimidazol-2-yl)methyl]-2-(morpholin-4- | |
| | yl)-8-(2,2,2-trifluoroethyl)pyrazolo[1,5-a][1,3,5]triazin-4-amine | |
| | ¹H-NMR (400 MHz, DMSO-d6) δ [ppm]: 1.905 (0.45), 2.318 (0.67), | |
| | 2.323 (1.48), 2.327 (2.12), 2.332 (1.48), 2.518 (8.06), 2.523 (5.35), | |
| | 2.540 (3.01), 2.660 (0.67), 2.665 (1.56), 2.669 (2.15), 2.674 (1.52), | |
| | 3.424 (3.04), 3.452 (7.98), 3.481 (14.37), 3.508 (4.05), 3.585 (9.50), | |
| | 3.816 (0.45), 4.858 (7.20), 4.870 (7.28), 7.750 (1.82), 7.913 (16.00), | |
| | 9.094 (3.01), 12.633 (0.89). | |
| 303 | 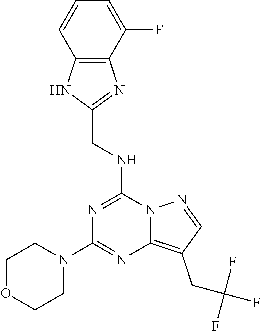 [SMILES: Fc1cccc2[nH]c(CNc3nc(N4CCOCC4)nc4c(CC(F)(F)F)cnn34)nc12] | 41% LC-MS (Method 1): Rₜ = 1.09 min; MS (ESIpos): m/z = 451 [M + H]⁺ |
| | N-[(4-fluoro-1H-benzimidazol-2-yl)methyl]-2-(morpholin-4-yl)-8- | |
| | (2,2,2-trifluoroethyl)pyrazolo[1,5-a][1,3,5]triazin-4-amine | |
| | ¹H-NMR (400 MHz, DMSO-d6) δ [ppm]: 1.903 (0.61), 2.318 (1.04), | |
| | 2.323 (2.35), 2.327 (3.30), 2.332 (2.35), 2.337 (0.96), 2.518 (11.30), | |
| | 2.523 (7.57), 2.540 (2.09), 2.660 (1.04), 2.665 (2.43), 2.669 (3.30), | |
| | 2.674 (2.35), 2.679 (1.04), 3.426 (3.83), 3.454 (9.74), 3.483 (16.00), | |
| | 3.511 (3.74), 3.601 (11.04), 4.871 (9.13), 4.885 (9.04), 6.915 (1.13), | |
| | 6.936 (1.74), 6.963 (1.57), 7.094 (1.74), 7.107 (2.09), 7.114 (3.65), | |
| | 7.126 (3.74), 7.135 (2.09), 7.147 (2.09), 7.237 (2.52), 7.256 (2.00), | |
| | 7.913 (15.65), 8.364 (0.87), 9.096 (2.00), 12.596 (1.91). | |
| 304 | 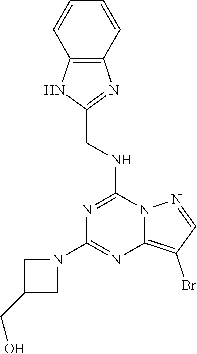 [SMILES: OCC1CN(c2nc(NCc3nc4ccccc4[nH]3)n3ncc(Br)c3n2)C1] | 84% LC-MS (Method 2): Rₜ = 0.87 min; MS (ESIpos): m/z = 429 [M + H]⁺ |
| | [1-(4-{[(1H-benzimidazol-2-yl)methyl]amino}-8- | |
| | bromopyrazolo[1,5-a][1,3,5]triazin-2-yl)azetidin-3-yl]methanol | |
| | ¹H-NMR (400 MHz, DMSO-d6) δ [ppm]: 2.074 (2.09), 2.327 (0.57), | |
| | 2.331 (0.41), 2.518 (4.21), 2.523 (2.88), 2.539 (0.79), 2.656 (0.81), | |
| | 2.659 (1.36), 2.665 (1.17), 2.669 (1.21), 2.673 (1.29), 2.694 (0.45), | |
| | 3.479 (2.34), 3.493 (3.93), 3.507 (2.41), 3.715 (1.24), 3.956 (1.05), | |
| | 4.750 (1.12), 4.763 (2.31), 4.776 (1.16), 4.860 (3.52), 4.869 (3.59), | |
| | 7.105 (0.50), 7.119 (1.93), 7.122 (1.59), 7.129 (2.48), 7.136 (2.72), | |
| | 7.141 (2.78), 7.152 (2.22), 7.166 (0.62), 7.404 (1.67), 7.421 (1.52), | |
| | 7.533 (1.60), 7.550 (1.53), 8.009 (16.00), 9.036 (1.43), 12.236 (2.07). | |
| 305 | 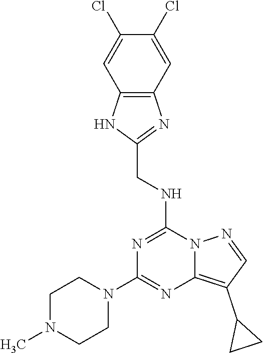 [SMILES: CN1CCN(c2nc(NCc3nc4cc(Cl)c(Cl)cc4[nH]3)n3ncc(C4CC4)c3n2)CC1] | 29% LC-MS (Method 2): Rₜ = 1.22 min; MS (ESIpos): m/z = 472 [M + H]⁺ |
| | 8-cyclopropyl-N-[(5,6-dichloro-1H-benzimidazol-2-yl)methyl]-2- | |
| | (4-methylpiperazin-1-yl)pyrazolo[1,5-a][1,3,5]triazin-4-amine | |
| | ¹H-NMR (400 MHz, DMSO-d6) δ [ppm]: 0.695 (1.07), 0.700 (0.79), | |
| | 0.707 (3.29), 0.712 (3.97), 0.720 (4.94), 0.725 (4.01), 0.733 (2.15), | |
| | 0.748 (0.63), 0.753 (0.72), 0.762 (2.00), 0.770 (3.83), 0.775 (2.65), | |
| | 0.783 (2.60), 0.787 (1.75), 0.791 (4.06), 0.796 (2.63), 0.802 (0.97), | |
| | 0.808 (1.27), 1.104 (2.95), 1.231 (0.47), 1.352 (0.55), 1.697 (0.64), | |
| | 1.710 (1.23), 1.718 (1.25), 1.724 (0.64), 1.730 (2.08), 1.739 (0.77), | |
| | 1.743 (1.16), 1.751 (1.15), 1.764 (0.52), 2.107 (16.00), 2.145 (3.29), | |
| | 2.323 (0.77), 2.327 (1.09), 2.332 (0.77), 2.518 (3.65), 2.523 (2.52), | |
| | 2.540 (4.19), 2.665 (0.79), 2.669 (1.09), 2.673 (0.77), 3.074 (0.91), | |
| | 3.552 (4.19), 4.820 (4.42), 4.835 (4.38), 7.657 (2.04), 7.692 (11.49), | |
| | 7.815 (2.00), 8.845 (1.11), 8.859 (2.42), 8.874 (1.11), 12.582 (2.15). | |
| 306 | 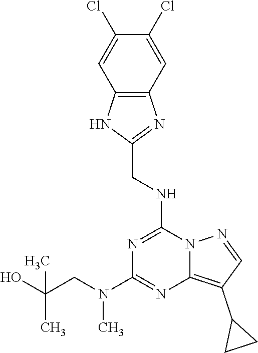 [SMILES: CN(CC(C)(C)O)c1nc(NCc2nc3cc(Cl)c(Cl)cc3[nH]2)n2ncc(C3CC3)c2n1] | 13% LC-MS (Method 1): Rₜ = 1.20 min; MS (ESIpos): m/z = 475 [M + H]⁺ |
| | 1-[(8-cyclopropyl-4-{[(5,6-dichloro-1H-benzimidazol-2- | |
| | yl)methyl]amino}pyrazolo[1,5-a][1,3,5]triazin-2- | |
| | yl)(methyl)amino]-2-methylpropan-2-ol | |
| | ¹H-NMR (400 MHz, DMSO-d6) δ [ppm]: 0.736 (10.95), 0.742 (16.00), | |
| | 0.747 (14.28), 0.755 (13.85), 0.761 (10.83), 0.785 (5.42), 1.075 | |
| | (2.09), 1.736 (1.11), 2.154 (0.68), 2.322 (1.85), 2.326 (2.52), 2.331 | |
| | (1.91), 2.522 (12.37), 2.539 (5.42), 2.664 (1.91), 2.668 (2.52), 2.673 | |
| | (1.85), 2.999 (1.23), 3.088 (4.92), 3.223 (0.43), 3.374 (3.75), 3.495 | |
| | (0.74), 4.325 (1.54), 4.787 (2.46), 5.020 (0.49), 7.634 (2.46), 7.678 | |
| | (3.63), 7.789 (2.52), 8.780 (1.23), 12.570 (2.77). | |
| 307 | 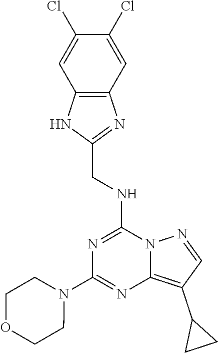 [SMILES: Clc1cc2nc(CNc3nc(N4CCOCC4)nc4c(C5CC5)cnn34)[nH]c2cc1Cl] | 23% LC-MS (Method 1): Rₜ = 1.15 min; MS (ESIpos): m/z = 443 [M + H]⁺ |
| | N-[(6-chloro-5-fluoro-1H-benzimidazol-2-yl)methyl]-8- | |
| | cyclopropyl-2-(morpholin-4-yl)pyrazolo[1,5-a][1,3,5]triazin-4- | |
| | amine | |
| | ¹H-NMR (400 MHz, DMSO-d6) δ [ppm]: 0.703 (1.84), 0.707 (1.60), | |
| | 0.716 (5.79), 0.721 (7.07), 0.728 (7.74), 0.733 (7.69), 0.741 (3.22), | |
| | 0.752 (1.25), 0.760 (1.79), 0.766 (3.41), 0.773 (6.63), 0.778 (4.63), | |
| | 0.787 (4.55), 0.794 (6.93), 0.799 (4.44), 0.811 (1.84), 1.703 (1.03), | |
| | 1.716 (2.03), 1.724 (2.14), 1.737 (3.41), 1.745 (1.46), 1.750 (1.95), | |
| | 1.758 (1.92), 1.771 (0.87), 2.327 (1.68), 2.447 (0.41), 2.540 (5.60), | |
| | 2.669 (1.57), 3.488 (9.29), 3.545 (9.18), 3.557 (10.37), 3.647 (0.95), | |
| | 3.659 (0.97), 3.718 (0.65), 4.823 (6.82), 4.837 (6.88), 7.433 (1.41), | |
| | 7.455 (1.41), 7.576 (1.81), 7.594 (2.63), 7.653 (0.81), 7.709 (16.00), | |
| | 7.737 (1.46), 7.754 (1.43), 8.891 (2.95), 12.513 (1.57), 12.552 (1.68). | |
| 308 | 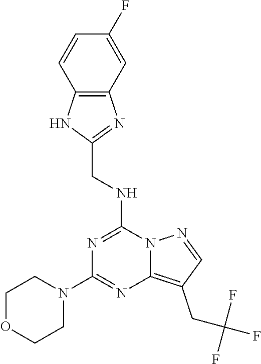 [SMILES: Fc1ccc2[nH]c(CNc3nc(N4CCOCC4)nc4c(CC(F)(F)F)cnn34)nc2c1] | 51% LC-MS (Method 1): Rₜ = 1.02 min; MS (ESIpos): m/z = 451 [M + H]⁺ |
| | N-[(5-fluoro-1H-benzimidazol-2-yl)methyl]-2-(morpholin-4-yl)-8- | |
| | (2,2,2-trifluoroethyl)pyrazolo[1,5-a][1,3,5]triazin-4-amine | |
| | ¹H-NMR (400 MHz, DMSO-d6) δ [ppm]: 1.728 (1.05), 2.327 (1.85), | |
| | 2.331 (1.37), 2.539 (3.13), 2.665 (1.41), 2.669 (1.88), 3.005 (0.83), | |
| | 3.015 (0.83), 3.425 (4.12), 3.454 (8.88), 3.483 (16.00), 3.600 (11.37), | |
| | 3.669 (0.80), 3.746 (0.51), 4.845 (8.50), 4.860 (8.56), 6.989 (2.11), | |
| | 7.220 (0.67), 7.329 (0.80), 7.393 (0.83), 7.523 (0.70), 7.837 (0.61), | |
| | 7.911 (15.20), 8.160 (3.42), 9.048 (1.95), 9.062 (3.90), 9.076 (2.08), | |
| | 12.392 (1.28). | |
| 309 | N-[(6-chloro-5-fluoro-1H-benzimidazol-2-yl)methyl]-8- | 27% |
| | cyclopropyl-2-(4-methylpiperazin-1-yl)pyrazolo[1,5- | LC-MS |
| | a][1,3,5]triazin-4-amine | (Method 2): |
| | ¹H-NMR (400 MHz, DMSO-d6) δ [ppm]: 0.695 (0.95), 0.700 (0.72), | Rₜ = 1.17 min; |
| | 0.707 (2.95), 0.713 (3.73), 0.720 (4.40), 0.726 (3.71), 0.733 (1.89), | MS (ESIpos): |
| | 0.748 (0.57), 0.753 (0.67), 0.762 (1.86), 0.769 (3.58), 0.775 (2.48), | m/z = 456 |
| | 0.783 (2.36), 0.787 (1.60), 0.791 (3.76), 0.796 (2.49), 0.802 (0.85), | [M + H]⁺ |
| | 0.808 (1.06), 1.697 (0.60), 1.710 (1.11), 1.718 (1.17), 1.724 (0.59), | |
| | 1.730 (1.94), 1.739 (0.70), 1.744 (1.09), 1.751 (1.08), 1.765 (0.49), | |
| | 2.108 (16.00), 2.151 (3.34), 2.323 (0.70), 2.327 (0.99), 2.332 (0.70), | |
| | 2.518 (3.49), 2.523 (2.28), 2.540 (0.41), 2.665 (0.70), 2.669 (0.98), | |
| | 2.673 (0.68), 3.559 (3.99), 4.811 (3.70), 4.826 (3.68), 7.580 (0.41), | |
| | 7.689 (10.44), 8.825 (0.83), 8.840 (1.68), 8.854 (0.78), 12.533 (0.64). | |
| 310 | 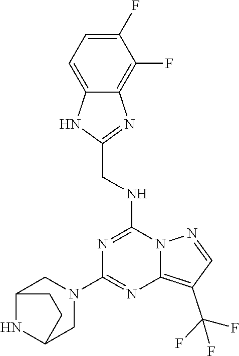 [SMILES: Fc1ccc2[nH]c(CNc3nc(N4CC5CCC(C4)N5)nc4c(C(F)(F)F)cnn34)nc2c1F] | 58% LC-MS (Method 2): Rₜ= 1.08 min; MS (ESpos): m/z = 480 [M + H]⁺ |
| | 2-(3,8-diazabicyclo[3.2.1]octan-3-yl)-N-[(4,5-difluoro-1H- | |
| | benzimidazol-2-yl)methyl]-8-(trifluoromethyl)pyrazolo[1,5- | |
| | a][1,3,5]triazin-4-amine | |
| | ¹H-NMR (400 MHz, DMSO-d6) δ [ppm]: −0.209 (0.79), 0.798 (0.62), | |
| | 0.815 (0.67), 0.822 (0.73), 0.840 (0.45), 0.851 (0.62), 0.905 (0.73), | |
| | 0.922 (0.45), 0.978 (1.40), 1.035 (0.51), 1.052 (0.73), 1.070 (0.45), | |
| | 1.232 (3.31), 1.256 (1.74), 1.302 (1.63), 1.331 (1.63), 1.474 (1.29), | |
| | 1.489 (1.29), 2.131 (0.51), 2.318 (1.07), 2.518 (12.35), 2.523 (8.08), | |
| | 2.660 (1.07), 2.749 (1.91), 2.781 (2.02), 2.856 (1.91), 2.887 (1.91), | |
| | 3.140 (1.96), 3.974 (1.74), 4.005 (1.74), 4.190 (1.91), 4.220 (1.85), | |
| | 4.827 (14.26), 7.150 (1.12), 7.172 (2.58), 7.178 (1.52), 7.189 (2.64), | |
| | 7.200 (3.09), 7.217 (3.03), 8.186 (0.45), 8.223 (16.00), 9.280 (0.56). | |
| 311 | 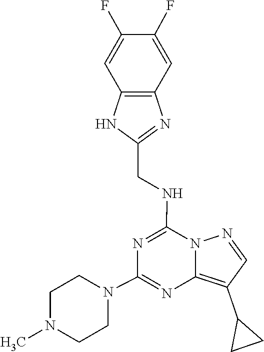 [SMILES: CN1CCN(c2nc(NCc3nc4cc(F)c(F)cc4[nH]3)n3ncc(C4CC4)c3n2)CC1] | 9% LC-MS (Method 2): Rₜ = 1.12 min; MS (ESIpos): m/z = 440 [M + H]⁺ |
| | 8-cyclopropyl-N-[(5,6-difluoro-1H-benzimidazol-2-yl)methyl]-2- | |
| | (4-methylpiperazin-1-yl)pyrazolo[1,5-a][1,3,5]triazin-4-amine | |
| | ¹H-NMR (400 MHz, DMSO-d6) δ [ppm]: 0.696 (1.53), 0.701 (1.16), | |
| | 0.708 (4.70), 0.713 (5.92), 0.721 (6.90), 0.726 (5.92), 0.734 (2.90), | |
| | 0.749 (0.89), 0.754 (1.07), 0.763 (3.02), 0.771 (5.56), 0.776 (3.85), | |
| | 0.784 (3.82), 0.788 (2.50), 0.791 (5.95), 0.797 (3.82), 0.802 (1.34), | |
| | 0.809 (1.77), 1.232 (0.95), 1.349 (0.43), 1.698 (0.95), 1.711 (1.80), | |
| | 1.719 (1.80), 1.725 (0.92), 1.732 (3.05), 1.740 (1.13), 1.744 (1.71), | |
| | 1.753 (1.92), 1.765 (0.76), 2.132 (9.13), 2.188 (3.60), 2.318 (0.67), | |
| | 2.323 (1.44), 2.327 (1.92), 2.332 (1.37), 2.337 (0.64), 2.518 (5.92), | |
| | 2.523 (4.06), 2.540 (0.70), 2.660 (0.61), 2.665 (1.37), 2.669 (1.86), | |
| | 2.673 (1.28), 2.679 (0.55), 3.566 (4.40), 3.572 (4.43), 4.804 (6.35), | |
| | 4.818 (6.29), 5.759 (7.54), 7.418 (2.41), 7.437 (2.69), 7.445 (2.66), | |
| | 7.463 (2.53), 7.563 (2.17), 7.582 (2.41), 7.591 (2.35), 7.610 (2.23), | |
| | 7.689 (16.00), 8.815 (1.44), 8.829 (2.96), 8.843 (1.34), 12.467 (3.82). | |
| 312 | 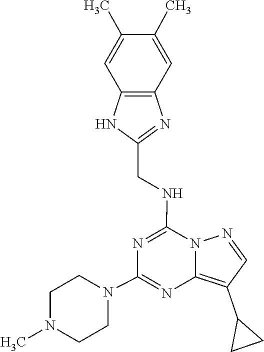 [SMILES: Cc1cc2nc(CNc3nc(N4CCN(C)CC4)nc4c(C5CC5)cnn34)[nH]c2cc1C] | 30% LC-MS (Method 2): Rₜ = 1.17 min; MS (ESIpos): m/z = 432 [M + H]⁺ |
| | 8-cyclopropyl-N-[(5,6-dimethyl-1H-benzimidazol-2-yl)methyl]-2- | |
| | (4-methylpiperazin-1-yl)pyrazolo[1,5-a][1,3,5]triazin-4-amine | |
| | ¹H-NMR (400 MHz, DMSO-d6) δ [ppm]: 0.698 (0.98), 0.703 (0.76), | |
| | 0.711 (3.37), 0.716 (4.26), 0.723 (4.78), 0.729 (4.35), 0.736 (2.06), | |
| | 0.749 (0.66), 0.756 (0.83), 0.763 (2.03), 0.771 (4.16), 0.776 (2.71), | |
| | 0.784 (2.70), 0.792 (4.17), 0.797 (2.73), 0.802 (0.95), 0.809 (1.15), | |
| | 1.232 (0.51), 17.01 (0.63), 1.714 (1.23), 1.722 (1.25), 1.734 (2.09), | |
| | 1.743 (0.83), 1.747 (1.21), 1.755 (1.18), 1.769 (0.57), 2.127 (16.00), | |
| | 2.200 (4.59), 2.270 (14.82), 2.322 (0.74), 2.326 (1.01), 2.331 (0.74), | |
| | 2.522 (3.81), 2.539 (1.40), 2.664 (0.61), 2.668 (0.89), 2.673 (0.63), | |
| | 3.597 (5.06), 4.788 (4.48), 4.802 (4.55), 5.758 (2.03), 7.158 (2.98), | |
| | 7.285 (2.96), 7.679 (11.03), 8.719 (1.09), 8.734 (2.38), 8.748 (1.12), | |
| | 11.962 (2.35). | |
| 313 | 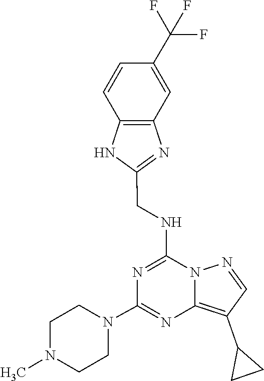 [SMILES: CN1CCN(c2nc(NCc3nc4cc(C(F)(F)F)ccc4[nH]3)n3ncc(C4CC4)c3n2)CC1] | 11% LC-MS (Method 2): Rₜ = 1.20 min; MS (ESIpos): m/z = 472 [M + H]⁺ |
| | 8-cyclopropyl-2-(4-methylpiperazin-1-yl)-N-{[5-(trifluoromethyl)- | |
| | 1H-benzimidazol-2-yl]methyl}pyrazolo[1,5-a][1,3,5]triazin-4- | |
| | amine | |
| | ¹H-NMR (400 MHz, DMSO-d6) δ [ppm]: 0.697 (1.27), 0.702 (1.04), | |
| | 0.709 (4.38), 0.714 (5.52), 0.722 (6.10), 0.727 (5.48), 0.735 (2.59), | |
| | 0.749 (0.85), 0.754 (1.06), 0.762 (2.63), 0.770 (5.35), 0.775 (3.48), | |
| | 0.783 (3.51), 0.791 (5.42), 0.796 (3.48), 0.808 (1.51), 1.232 (1.02), | |
| | 1.698 (0.83), 1.711 (1.57), 1.719 (1.61), 1.732 (2.70), 1.740 (1.10), | |
| | 1.745 (1.59), 1.752 (1.64), 1.766 (0.74), 2.069 (16.00), 2.327 (1.27), | |
| | 2.331 (0.93), 2.522 (5.27), 2.539 (1.55), 2.665 (0.91), 2.668 (1.25), | |
| | 2.673 (0.93), 3.538 (5.61), 4.860 (5.61), 4.875 (5.65), 5.759 (2.72), | |
| | 7.440 (0.79), 7.459 (2.00), 7.478 (1.61), 7.588 (1.72), 7.609 (1.32), | |
| | 7.698 (11.05), 7.718 (1.13), 7.735 (2.02), 7.896 (2.36), 8.899 (1.85), | |
| | 12.698 (1.74). | |
| 314 | 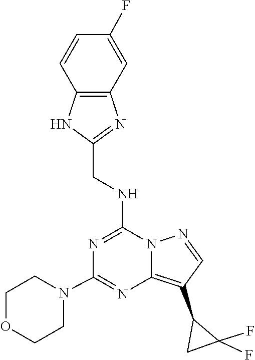 [SMILES: Fc1ccc2[nH]c(CNc3nc(N4CCOCC4)nc4c([C@@H]5CC5(F)F)cnn34)nc2c1] | 10% LC-MS (Method 2): Rₜ = 1.11 min; MS (ESIpos): m/z = 445 [M + H]⁺ |
| | 8-[(1R or S)-2,2-difluorocyclopropyl]-N-[(5-fluoro-1H- | |
| | benzimidazol-2-yl)methyl]-2-(morpholin-4-yl)pyrazolo[1,5- | |
| | a][1,3,5]triazin-4-amine (Enantiomer 1) | |
| | ¹H-NMR (400 MHz, DMSO-d6) δ [ppm]: 0.776 (0.70), 0.795 (1.31), | |
| | 0.814 (0.77), 0.819 (0.70), 0.836 (0.85), 0.844 (0.70), 0.852 (0.93), | |
| | 0.862 (1.08), 0.881 (0.46), 1.006 (1.24), 1.035 (1.16), 1.085 (2.71), | |
| | 1.100 (0.54), 1.137 (2.32), 1.169 (0.46), 1.233 (3.63), 1.259 (6.57), | |
| | 1.353 (0.46), 1.376 (0.46), 1.392 (0.54), 1.422 (0.46), 1.440 (0.39), | |
| | 1.543 (0.46), 1.875 (0.46), 1.894 (1.00), 1.907 (1.47), 1.924 (1.47), | |
| | 1.938 (1.70), 1.957 (1.08), 1.963 (1.08), 1.971 (1.16), 1.982 (1.70), | |
| | 1.994 (2.01), 2.006 (1.16), 2.014 (1.78), 2.026 (1.39), 2.034 (0.70), | |
| | 2.045 (0.46), 2.116 (0.93), 2.318 (1.39), 2.323 (3.25), 2.327 (4.71), | |
| | 2.332 (3.32), 2.336 (1.47), 2.518 (16.00), 2.523 (10.90), 2.660 (1.47), | |
| | 2.665 (3.48), 2.669 (4.87), 2.673 (3.40), 2.679 (1.62), 2.685 (1.55), | |
| | 2.705 (1.47), 2.715 (2.78), 2.735 (2.63), 2.745 (1.47), 2.765 (1.24), | |
| | 3.490 (6.11), 3.586 (7.42), 4.839 (4.17), 5.759 (3.17), 6.950 (0.46), | |
| | 6.973 (1.39), 6.996 (1.47), 7.019 (0.62), 7.195 (0.93), 7.213 (0.85), | |
| | 7.323 (1.00), 7.344 (1.00), 7.375 (0.85), 7.388 (0.93), 7.397 (0.93), | |
| | 7.409 (0.77), 7.512 (0.70), 7.524 (0.85), 7.546 (0.62), 7.846 (11.59), | |
| | 9.003 (1.47), 10.514 (0.39), 10.850 (1.93), 12.383 (1.55). | |
| 315 | 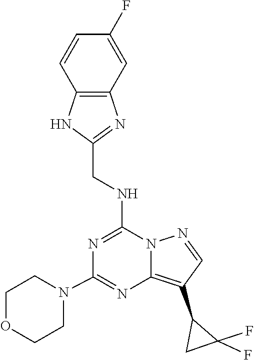 [SMILES: Fc1ccc2[nH]c(CNc3nc(N4CCOCC4)nc4c([C@@H]5CC5(F)F)cnn34)nc2c1] | 12% LC-MS (Method 2): Rₜ = 1.11 min; MS (ESIpos): m/z = 445 [M + H]⁺ |
| | 8-[(1R or S)-2,2-difluorocyclopropyl]-N-[(5-fluoro-1H- | |
| | benzimidazol-2-yl)methyl]-2-(morpholin-4-yl)pyrazolo[1,5- | |
| | a][1,3,5]triazin-4-amine (Enantiomer 2) | |
| | ¹H-NMR (400 MHz, DMSO-d6) δ [ppm]: 0.776 (0.85), 0.795 (1.77), 0.814 (1.00), | |
| | 0.819 (0.85), 0.836 (1.08), 0.844 (0.77), 0.852 (1.08), 0.862 (1.31), 0.880 (0.46), | |
| | 1.006 (1.54), 1.035 (1.54), 1.050 (0.46), 1.085 (3.23), 1.100 (0.62), 1.137 (2.62), | |
| | 1.150 (0.85), 1.169 (0.77), 1.204 (0.85), 1.233 (4.00), 1.259 (7.31), 1.285 (0.77), | |
| | 1.357 (0.54), 1.382 (0.62), 1.392 (0.62), 1.422 (0.62), 1.444 (0.54), 1.544 (0.46), | |
| | 1.610 (0.38), 1.752 (0.38), 1.875 (0.62), 1.894 (1.15), 1.907 (2.38), 1.924 (1.77), | |
| | 1.938 (2.15), 1.957 (1.46), 1.963 (1.46), 1.971 (1.54), 1.982 (2.08), 1.994 (2.46), | |
| | 2.006 (1.54), 2.014 (2.15), 2.026 (1.77), 2.034 (0.92), 2.045 (0.69), 2.062 (0.92), | |
| | 2.116 (1.15), 2.318 (1.46), 2.323 (3.31), 2.327 (4.77), 2.332 (3.23), 2.336 (1.46), | |
| | 2.458 (1.00), 2.462 (1.31), 2.466 (1.54), 2.518 (16.00), 2.523 (10.92), 2.537 (0.77), | |
| | 2.541 (0.46), 2.660 (1.46), 2.665 (3.46), 2.669 (4.85), 2.673 (3.38), 2.679 (1.62), | |
| | 2.685 (1.69), 2.705 (1.69), 2.715 (3.23), 2.735 (3.08), 2.745 (1.77), 2.765 (1.46), | |
| | 2.995 (1.54), 3.492 (7.00), 3.583 (8.23), 4.830 (4.00), 4.840 (5.46), 4.854 (4.23), | |
| | 5.759 (7.77), 6.943 (1.15), 6.950 (1.23), 6.965 (1.38), 6.969 (1.54), 6.974 (2.38), | |
| | 6.980 (1.62), 6.990 (1.46), 6.996 (2.62), 7.002 (1.92), 7.005 (1.62), 7.020 (1.31), | |
| | 7.026 (1.38), 7.190 (2.00), 7.196 (2.00), 7.212 (1.92), 7.219 (1.92), 7.318 (1.92), | |
| | 7.324 (2.08), 7.343 (1.92), 7.349 (2.00), 7.375 (2.08), 7.387 (2.23), 7.397 (2.15), | |
| | 7.409 (1.85), 7.512 (1.62), 7.524 (1.77), 7.533 (1.69), 7.546 (1.54), 7.846 (12.46), | |
| | 7.880 (0.38), 7.897 (0.69), 8.987 (0.92), 9.001 (2.62), 9.015 (2.62), 9.029 (0.85), | |
| | 10.514 (0.62), 10.634 (0.62), 10.850 (2.62), 12.362 (2.46), 12.382 (2.54). | |
| 316 | 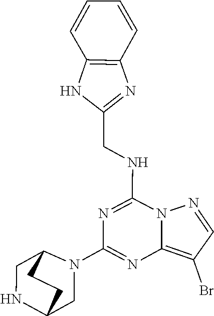 [SMILES: Brc1cnn2c(NCc3nc4ccccc4[nH]3)nc(N3C[C@H]4CC[C@@H]3CN4)nc12] | 28% LC-MS (Method 2): Rₜ = 0.94 min; MS (ESIpos): m/z = 454 [M + H]⁺ |
| | N-[(1H-benzimidazol-2-yl)methyl]-8-bromo-2-{[1R or S- | |
| | (1R*4R*)]-2,5-diazabicyclo[2.2.2]octan-2-yl}pyrazolo[1,5- | |
| | a][1,3,5]triazin-4-amine (Enantiomer 1) | |
| | ¹H-NMR (400 MHz, DMSO-d6) δ [ppm]: 1.231 (2.67), 1.352 (2.60), | |
| | 1.534 (5.68), 1.723 (2.11), 1.752 (1.82), 1.793 (6.81), 1.876 (1.12), | |
| | 2.326 (6.11), 2.466 (2.04), 2.669 (4.14), 2.753 (8.14), 2.909 (3.02), | |
| | 2.946 (4.42), 2.984 (2.88), 3.031 (2.32), 3.057 (1.33), 3.428 (4.98), | |
| | 3.466 (3.30), 3.495 (6.95), 3.525 (1.96), 3.548 (4.21), 3.577 (2.67), | |
| | 3.675 (0.98), 4.379 (4.28), 4.592 (2.95), 4.807 (14.46), 4.844 (1.47), | |
| | 4.900 (8.91), 7.124 (9.75), 7.395 (2.67), 7.526 (3.23), 7.981 (16.00), | |
| | 7.986 (11.37), 8.016 (1.68), 8.524 (0.70), 12.252 (2.46). | |
| 317 | 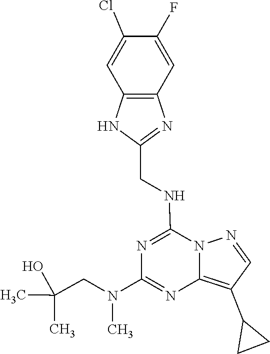 [SMILES: CN(CC(C)(C)O)c1nc(NCc2nc3cc(F)c(Cl)cc3[nH]2)n2ncc(C3CC3)c2n1] | 8% LC-MS (Method 1): Rₜ = 1.12 min; MS (ESIpos): m/z = 459 [M + H]⁺ |
| | 1-[(4-{[(6-chloro-5-fluoro-1H-benzimidazol-2-yl)methyl]amino}-8- | |
| | cyclopropylpyrazolo[1,5-a][1,3,5]triazin-2-yl)(methyl)amino]-2- | |
| | methylpropan-2-ol | |
| | ¹H-NMR (400 MHz, DMSO-d6) δ [ppm]: 0.723 (2.54), 0.737 (10.39), | |
| | 0.743 (15.48), 0.748 (14.21), 0.750 (14.73), 0.755 (16.00), 0.761 | |
| | (12.43), 0.786 (5.53), 0.847 (0.52), 0.859 (0.90), 0.864 (0.97), 0.872 | |
| | (0.90), 0.877 (0.90), 0.886 (0.52), 0.955 (0.60), 0.963 (0.97), 0.969 | |
| | (0.82), 0.976 (0.67), 0.984 (1.05), 0.990 (0.82), 1.001 (0.60), 1.077 | |
| | (2.02), 1.113 (1.72), 1.734 (1.05), 1.995 (0.45), 2.102 (0.45), 2.318 | |
| | 0.90), 2.323 (2.02), 2.327 (2.84), 2.332 (1.94), 2.337 (0.82), 2.518 | |
| | (9.12), 2.523 (6.13), 2.540 (2.09), 2.660 (0.90), 2.665 (2.02), 2.669 | |
| | (2.92), 2.673 (1.94), 2.679 (0.90), 2.825 (0.52), 3.008 (1.35), 3.090 | |
| | (4.86), 3.224 (2.92), 3.226 (3.14), 3.388 (2.77), 3.496 (0.82), 4.326 | |
| | (1.72), 4.777 (2.32), 5.004 (0.82), 5.018 (0.90), 7.404 (0.75), 7.426 | |
| | (0.90), 7.440 (0.67), 7.463 (0.45), 7.487 (0.45), 7.546 (1.42), 7.566 | |
| | (1.79), 7.597 (0.60), 7.606 (0.67), 7.622 (0.82), 7.677 (3.51), 7.708 | |
| | (1.12), 7.723 (1.12), 7.766 (0.45), 7.784 (0.37), 8.135 (0.52), 8.205 | |
| | (1.79), 8.766 (1.12), 12.497 (1.50), 12.540 (1.72). | |
| 318 | 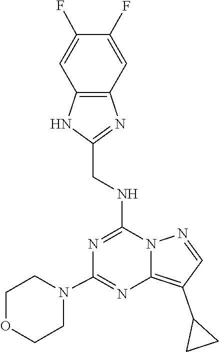 [SMILES: Fc1cc2nc(CNc3nc(N4CCOCC4)nc4c(C5CC5)cnn34)[nH]c2cc1F] | 20% LC-MS (Method 1): Rₜ = 1.08 min; MS (ESIpos): m/z = 427 [M + H]⁺ |
| | 8-cyclopropyl-N-[(5,6-difluoro-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: 0.703 (1.60), 0.707 (1.07), | |
| | 0.715 (4.55), 0.720 (5.91), 0.727 (6.93), 0.733 (6.56), 0.741 (2.99), | |
| | 0.752 (0.94), 0.760 (1.27), 0.765 (2.87), 0.772 (5.46), 0.778 (3.77), | |
| | 0.786 (3.90), 0.790 (2.46), 0.793 (5.62), 0.799 (3.65), 0.805 (1.15), | |
| | 0.810 (1.60), 1.703 (0.94), 1.716 (1.72), 1.723 (1.81), 1.737 (2.83), | |
| | 1.745 (1.07), 1.749 (1.68), 1.758 (1.60), 1.770 (0.78), 2.323 (0.66), | |
| | 2.327 (0.98), 2.332 (0.70), 2.518 (4.02), 2.523 (2.63), 2.539 (0.90), | |
| | 2.665 (0.70), 2.669 (0.98), 2.673 (0.70), 3.492 (6.69), 3.551 (6.56), | |
| | 3.563 (7.67), 4.813 (5.50), 4.828 (5.54), 7.421 (1.60), 7.440 (1.85), | |
| | 7.448 (1.85), 7.466 (1.64), 7.563 (1.76), 7.582 (1.93), 7.591 (1.93), | |
| | 7.610 (1.81), 7.705 (16.00), 8.138 (0.78), 8.857 (1.39), 8.872 (2.99), | |
| | 8.886 (1.39), 12.467 (3.04). | |
| 319 | 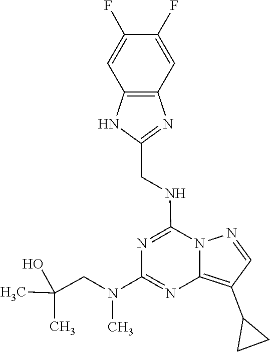 [SMILES: CN(CC(C)(C)O)c1nc(NCc2nc3cc(F)c(F)cc3[nH]2)n2ncc(C3CC3)c2n1] | 6% LC-MS (Method 1): Rₜ = 1.04 min; MS (ESIpos): m/z = 443 [M + H]⁺ |
| | 1-[(8-cyclopropyl-4-{[(5,6-difluoro-1H-benzimidazol-2- | |
| | yl)methyl]amino}pyrazolo[1,5-a][1,3,5]triazin-2- | |
| | yl)(methyl)amino]-2-methylpropan-2-ol | |
| | ¹H-NMR (400 MHz, DMSO-d6) δ [ppm]: 0.722 (1.91), 0.737 (8.06), | |
| | 0.743 (12.41), 0.750 (12.56), 0.755 (16.00), 0.761 (13.99), 0.786 | |
| | (5.60), 1.078 (2.34), 1.137 (0.55), 1.232 (1.07), 1.729 (1.03), 2.332 | |
| | (2.29), 2.336 (0.99), 2.518 (12.20), 2.523 (8.10), 2.539 (0.72), 2.678 | |
| | (1.00), 3.093 (4.59), 3.400 (2.60), 3.504 (0.71), 4.011 (0.70), 4.328 | |
| | (1.35), 4.765 (2.14), 7.418 (1.24), 7.562 (1.24), 7.674 (3.26), 8.177 | |
| | (0.59), 8.747 (1.11), 12.455 (2.49). | |
| 320 | 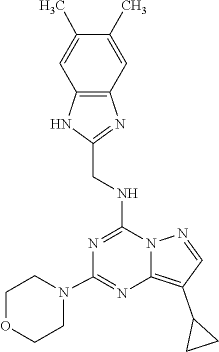 [SMILES: Cc1cc2nc(CNc3nc(N4CCOCC4)nc4c(C5CC5)cnn34)[nH]c2cc1C] | 36% LC-MS (Method 1): Rₜ = 0.97 min; MS (ESIneg): m/z = 417 [M − H]⁻ |
| | 8-cyclopropyl-N-[(5,6-dimethyl-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: 0.707 (0.52), 0.720 (1.65), | |
| | 0.725 (2.13), 0.732 (2.35), 0.737 (2.21), 0.745 (0.97), 0.763 (0.56), | |
| | 0.767 (1.03), 0.774 (1.84), 0.779 (1.51), 0.788 (1.24), 0.795 (2.00), | |
| | 0.800 (1.29), 0.812 (0.52), 1.720 (0.59), 1.728 (0.61), 1.741 (0.99), | |
| | 1.754 (0.58), 1.761 (0.56), 2.269 (16.00), 2.327 (0.43), 2.522 (1.17), | |
| | 2.669 (0.43), 3.514 (3.01), 3.523 (2.63), 3.578 (2.94), 3.590 (3.41), | |
| | 3.599 (1.86), 4.795 (2.42), 4.810 (2.42), 7.700 (5.21), 8.136 (2.53), | |
| | 8.775 (0.61), 8.790 (1.33), 8.805 (0.60). | |
| 321 | 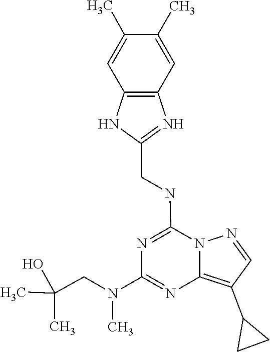 | 28% LC-MS (Method 1): Rₜ = 0.96 min; m/z = 435 [M + H]⁺ |
| | 1-[(8-cyclopropyl-4-{[(5,6-dimethyl-1H-benzimidazol-2- | |
| | yl)methyl]amino}pyrazolo[1,5-a][1,3,5]triazin-2- | |
| | yl)(methyl)amino]-2-methylpropan-2-ol | |
| | ¹H-NMR (400 MHz, DMSO-d6) δ [ppm]: 0.724 (0.28), 0.739 (1.71), | |
| | 0.745 (2.87), 0.749 (1.89), 0.752 (2.04), 0.757 (3.07), 0.763 (2.68), | |
| | 0.785 (1.56), 0.857 (2.13), 1.031 (0.31), 1.087 (0.73), 1.145 (0.28), | |
| | 1.735 (0.32), 2.075 (0.18), 2.266 (16.00), 2.318 (0.32), 2.323 (0.53), | |
| | 2.327 (0.67), 2.332 (0.49), 2.518 (2.68), 2.523 (1.62), 2.539 (0.26), | |
| | 2.665 (0.45), 2.669 (0.63), 2.673 (0.46), 3.101 (1.56), 3.442 (0.86), | |
| | 4.361 (0.26), 4.754 (0.67), 7.157 (0.41), 7.267 (0.43), 7.665 (0.88), | |
| | 8.142 (2.84), 8.656 (0.32), 11.952 (0.34). | |
| 322 | 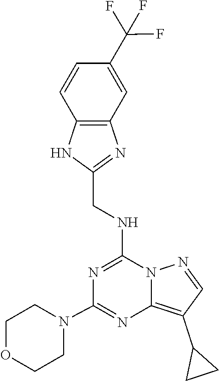 [SMILES: FC(F)(F)c1ccc2[nH]c(CNc3nc(N4CCOCC4)nc4c(C5CC5)cnn34)nc2c1] | 13% LC-MS (Method 1): Rₜ = 1.17 min; MS (ESIpos): m/z = 459 [M + H]⁺ |
| | 8-cyclopropyl-2-(morpholin-4-yl)-N-{[5-(trifluoromethyl)-1H- | |
| | benzimidazol-2-yl]methyl}pyrazolo[1,5-a][1,3,5]triazin-4-amine | |
| | ¹H-NMR (400 MHz, DMSO-d6) δ [ppm]: 0.707 (1.66), 0.719 (4.93), | |
| | 0.724 (6.28), 0.732 (6.97), 0.737 (6.83), 0.745 (3.10), 0.755 (1.21), | |
| | 0.763 (1.55), 0.768 (3.31), 0.775 (5.72), 0.781 (4.28), 0.789 (4.03), | |
| | 0.793 (2.90), 0.797 (5.93), 0.802 (4.00), 0.808 (1.34), 0.814 (1.79), | |
| | 1.707 (0.90), 1.720 (1.72), 1.728 (1.86), 1.741 (2.93), 1.749 (1.17), | |
| | 1.754 (1.72), 1.762 (1.66), 1.775 (0.83), 2.323 (1.45), 2.327 (2.00), | |
| | 2.331 (1.52), 2.518 (16.00), 2.523 (11.07), 2.539 (3.79), 2.665 (1.48), | |
| | 2.669 (2.10), 2.673 (1.52), 3.461 (6.86), 3.532 (7.28), 3.543 (8.21), | |
| | 3.647 (0.55), 3.659 (0.48), 4.878 (5.69), 4.892 (5.83), 7.463 (1.69), | |
| | 7.481 (1.59), 7.595 (1.24), 7.615 (1.03), 7.653 (0.59), 7.719 (14.72), | |
| | 7.742 (1.62), 7.899 (1.86), 8.929 (1.34), 8.943 (2.72), 8.957 (1.38), | |
| | 12.698 (1.79). | |
| 323 | 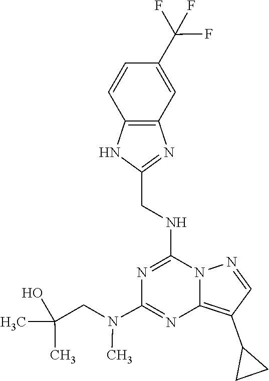 [SMILES: CN(CC(C)(C)O)c1nc(NCc2nc3cc(C(F)(F)F)ccc3[nH]2)n2ncc(C3CC3)c2n1] | 1% LC-MS (Method 1): Rₜ = 1.14 min; MS (ESIneg): m/z = 473 [M − H]⁻ |
| | 1-{[8-cyclopropyl-4-({[5-(trifluoromethyl)-1H-benzimidazol-2- | |
| | yl]methyl}amino)pyrazolo[1,5-a][1,3,5]triazin-2- | |
| | yl](methyl)amino}-2-methylpropan-2-ol | |
| | ¹H-NMR (400 MHz, DMSO-d6) δ [ppm]: 0.693 (5.26), 0.726 (1.44), | |
| | 0.740 (3.99), 0.746 (5.96), 0.753 (4.45), 0.759 (5.76), 0.764 (5.36), | |
| | 0.788 (3.20), 1.077 (1.32), 1.232 (1.15), 2.518 (16.00), 2.522 (10.20), | |
| | 2.539 (7.00), 3.081 (3.22), 4.296 (0.76), 4.830 (1.55), 7.446 (1.53), | |
| | 7.466 (2.03), 7.687 (2.50), 7.859 (0.43), 8.813 (0.77), 12.694 (0.83). | |
| 324 | 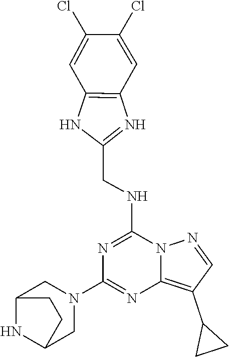 [SMILES: Clc1cc2nc(CNc3nc(N4CC5CCC(C4)N5)nc4c(C5CC5)cnn34)[nH]c2cc1Cl] | 32% LC-MS (Method 2): Rₜ = 1.15 min; MS (ESIpos): m/z = 484 [M + H]⁺ |
| | 8-cyclopropyl-2-(3,8-diazabicyclo[3.2.1]octan-3-yl)-N-[(5,6- | |
| | dichloro-1H-benzimidazol-2-yl)methyl]pyrazolo[1,5- | |
| | a][1,3,5]triazin-4-amine | |
| | ¹H-NMR (400 MHz, DMSO-d6) δ [ppm]: 0.712 (6.98), 0.719 (7.16), | |
| | 0.724 (6.98), 0.732 (3.10), 0.759 (3.13), 0.767 (6.50), 0.779 (4.27), | |
| | 0.787 (6.40), 0.804 (1.58), 0.851 (0.48), 1.035 (2.20), 1.052 (4.40), | |
| | 1.069 (2.24), 1.163 (1.89), 1.232 (3.68), 1.348 (1.34), 1.397 (1.03), | |
| | 1.515 (1.10), 1.686 (1.10), 1.698 (1.96), 1.706 (2.10), 1.718 (3.23), | |
| | 1.732 (1.96), 1.739 (1.75), 1.751 (2.24), 1.904 (0.55), 2.183 (0.69), | |
| | 2.268 (0.41), 2.322 (1.41), 2.327 (1.96), 2.332 (1.41), 2.522 (7.43), | |
| | 2.539 (4.40), 2.665 (1.51), 2.669 (2.06), 2.673 (1.51), 2.928 (1.93), | |
| | 3.354 (6.78), 3.411 (5.13), 3.428 (5.44), 3.445 (4.78), 3.463 (3.20), | |
| | 4.175 (1.24), 4.799 (6.43), 5.758 (2.55), 7.702 (16.00), 8.940 (1.27). | |
| 325 | 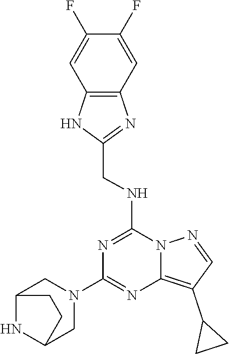 [SMILES: Fc1cc2nc(CNc3nc(N4CC5CCC(C4)N5)nc4c(C5CC5)cnn34)[nH]c2cc1F] | 19% LC-MS (Method 2): Rₜ = 1.05 min; MS (ESIpos): m/z = 452 [M + H]⁺ |
| | 8-cyclopropyl-2-(3,8-diazabicyclo[3.2.1]octan-3-yl)-N-[(5,6- | |
| | difluoro-1H-benzimidazol-2-yl)methyl]pyrazolo[1,5- | |
| | a][1,5]triazin-4-amine | |
| | ¹H-NMR (400 MHz, DMSO-d6) δ [ppm]: 0.688 (1.38), 0.701 (4.36), | |
| | 0.706 (5.67), 0.713 (6.11), 0.718 (5.75), 0.726 (2.62), 0.736 (0.95), | |
| | 0.745 (1.49), 0.748 (2.47), 0.755 (5.02), 0.760 (3.85), 0.769 (3.16), | |
| | 0.777 (5.53), 0.782 (3.38), 0.789 (1.13), 0.793 (1.20), 1.035 (4.62), | |
| | 1.052 (8.11), 1.070 (4.84), 1.233 (1.89), 1.349 (1.24), 1.676 (0.91), | |
| | 1.689 (1.75), 1.696 (1.78), 1.709 (2.73), 1.718 (1.09), 1.722 (1.67), | |
| | 1.730 (1.60), 1.743 (0.80), 1.751 (2.58), 1.897 (0.40), 2.322 (1.45), | |
| | 2.327 (2.07), 2.332 (1.53), 2.336 (0.69), 2.518 (8.91), 2.523 (5.53), | |
| | 2.539 (3.78), 2.660 (0.76), 2.664 (1.56), 2.669 (2.18), 2.673 (1.60), | |
| | 2.678 (0.80), 2.780 (1.42), 3.405 (0.69), 3.422 (1.24), 3.435 (1.27), | |
| | 3.440 (1.20), 3.452 (1.16), 3.469 (0.44), 4.067 (0.69), 4.344 (0.47), | |
| | 4.357 (0.91), 4.370 (0.47), 4.767 (4.76), 7.518 (0.76), 7.663 (16.00), | |
| | 7.947 (0.55), 8.790 (1.35), 12.461 (0.51). | |
| 326 | 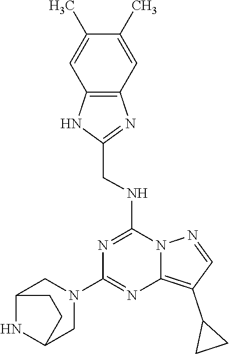 [SMILES: Cc1cc2nc(CNc3nc(N4CC5CCC(C4)N5)nc4c(C5CC5)cnn34)[nH]c2cc1C] | 19% LC-MS (Method 2): Rₜ = 1.10 min; MS (ESIpos): m/z = 444 [M + H]⁺ |
| | 8-cyclopropyl-2-(3,8-diazabicyclo[3.2.1]octan-3-yl)-N-[(5,6- | |
| | dimethyl-1H-benzimidazol-2-yl)methyl]pyrazolo[1,5- | |
| | a][1,5]triazin-4-amine | |
| | ¹H-NMR (400 MHz, DMSO-d6) δ [ppm]: 0.695 (1.18), 0.708 (3.52), | |
| | 0.713 (4.52), 0.720 (5.15), 0.726 (4.24), 0.733 (2.15), 0.740 (0.88), | |
| | 0.752 (2.24), 0.759 (4.06), 0.765 (3.15), 0.773 (2.48), 0.781 (4.39), | |
| | 0.786 (2.73), 0.793 (0.85), 0.798 (1.06), 1.035 (2.58), 1.053 (4.82), | |
| | 1.070 (2.61), 1.232 (1.39), 1.348 (0.70), 1.447 (1.06), 1.683 (0.76), | |
| | 1.696 (1.39), 1.704 (1.42), 1.716 (2.15), 1.725 (0.88), 1.730 (1.33), | |
| | 1.737 (1.27), 1.751 (2.45), 2.263 (15.91), 2.271 (16.00), 2.318 (0.64), | |
| | 2.323 (1.24), 2.327 (1.76), 2.332 (1.27), 2.336 (0.70), 2.518 (5.36), | |
| | 2.523 (3.61), 2.539 (0.64), 2.660 (0.55), 2.665 (1.21), 2.669 (1.70), | |
| | 2.673 (1.18), 2.678 (0.55), 2.787 (1.58), 2.817 (1.64), 3.423 (0.91), | |
| | 3.434 (0.79), 3.440 (0.73), 3.451 (0.64), 4.109 (1.24), 4.136 (1.21), | |
| | 4.763 (4.48), 7.153 (4.18), 7.282 (4.15), 7.657 (12.85), 7.941 (0.42), | |
| | 0.868 (0.76), 11.950 (2.39). | |
| 327 | 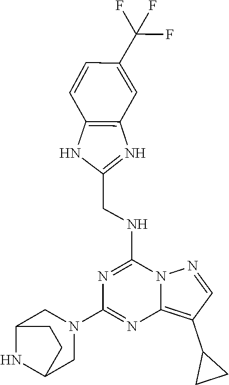 [SMILES: FC(F)(F)c1ccc2[nH]c(CNc3nc(N4CC5CCC(C4)N5)nc4c(C5CC5)cnn34)nc2c1] | 15% LC-MS (Method 2): Rₜ = 1.12 min; MS (ESIpos): m/z = 484 [M + H]⁺ |
| | 8-cyclopropyl-2-(3,8-diazabicyclo[3.2.1]octan-3-yl)-N-{[5- | |
| | (trifluoromethyl)-1H-benzimidazol-2-yl]methyl}pyrazolo[1,5- | |
| | a][1,3,5]triazin-4-amine | |
| | ¹H-NMR (400 MHz, DMSO-d6) δ [ppm]: 0.691 (1.31), 0.704 (4.16), | |
| | 0.709 (5.41), 0.716 (5.80), 0.722 (5.32), 0.729 (2.56), 0.738 (0.92), | |
| | 0.750 (2.37), 0.757 (4.74), 0.763 (4.01), 0.771 (2.90), 0.779 (5.37), | |
| | 0.784 (3.34), 0.791 (1.06), 0.796 (1.21), 1.035 (0.82), 1.053 (1.35), | |
| | 1.070 (0.82), 1.233 (1.93), 1.349 (0.97), 1.679 (0.92), 1.691 (1.69), | |
| | 1.699 (1.69), 1.712 (2.61), 1.720 (1.06), 1.725 (1.64), 1.733 (1.60), | |
| | 1.746 (0.82), 1.751 (2.22), 1.902 (0.44), 2.318 (0.82), 2.322 (1.93), | |
| | 2.327 (2.80), 2.332 (1.98), 2.336 (0.87), 2.453 (0.44), 2.457 (0.77), | |
| | 2.462 (1.02), 2.466 (1.21), 2.518 (9.67), 2.523 (6.28), 2.532 (0.68), | |
| | 2.536 (0.48), 2.540 (1.02), 2.660 (0.97), 2.665 (2.08), 2.669 (2.95), | |
| | 2.673 (2.13), 2.679 (1.02), 2.756 (1.16), 3.988 (0.44), 4.831 (4.21), | |
| | 5.759 (1.45), 7.445 (2.08), 7.466 (2.66), 7.623 (0.58), 7.677 (16.00), | |
| | 7.877 (0.48), 7.960 (0.53), 8.865 (1.35), 12.688 (0.44). | |
| 328 | 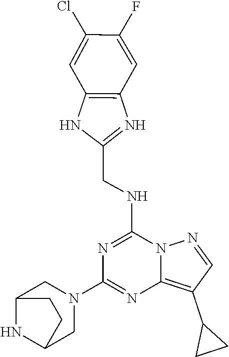 [SMILES: Fc1cc2nc(CNc3nc(N4CC5CCC(C4)N5)nc4c(C5CC5)cnn34)[nH]c2cc1Cl] | 89% LC-MS (Method 2): Rₜ = 1.09 min; MS (ESIpos): m/z = 468 [M + H]⁺ |
| | N-[(6-chloro-5-fluoro-1H-benzimidazol-2-yl)methyl]-8- | |
| | cyclopropyl-2-[(1R,5S)-3,8-diazabicyclo[3.2.1]octan-3- | |
| | yl]pyrazolo[1,5-a][1,3,5]triazin-4-amine | |
| | ¹H-NMR (400 MHz, DMSO-d6) δ [ppm]: 0.689 (1.30), 0.701 (3.97), | |
| | 0.706 (5.26), 0.713 (5.80), 0.719 (5.67), 0.727 (2.43), 0.736 (0.84), | |
| | 0.745 (1.24), 0.750 (2.21), 0.757 (4.64), 0.762 (3.62), 0.770 (2.91), | |
| | 0.777 (5.13), 0.783 (3.32), 0.790 (1.08), 0.795 (1.19), 1.035 (0.78), | |
| | 1.052 (1.21), 1.070 (0.81), 1.232 (1.35), 1.349 (1.00), 1.676 (0.89), | |
| | 1.689 (1.62), 1.697 (1.70), 1.710 (2.56), 1.718 (1.05), 1.723 (1.56), | |
| | 1.730 (1.54), 1.744 (0.76), 1.751 (2.24), 2.318 (0.46), 2.323 (1.05), | |
| | 2.327 (1.51), 2.332 (1.08), 2.336 (0.49), 2.518 (6.39), 2.523 (4.02), | |
| | 2.539 (5.53), 2.660 (0.51), 2.665 (1.13), 2.669 (1.59), 2.673 (1.16), | |
| | 2.678 (0.57), 2.782 (1.24), 4.071 (0.57), 4.775 (5.88), 5.759 (4.75), | |
| | 7.502 (0.51), 7.668 (16.00), 8.819 (0.78). | |
| 329 | 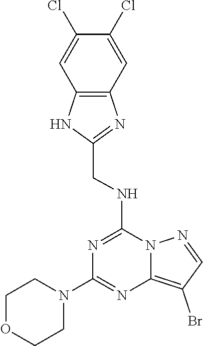 [SMILES: Clc1cc2nc(CNc3nc(N4CCOCC4)nc4c(Br)cnn34)[nH]c2cc1Cl] | 17% LC-MS (Method 1): Rₜ = 1.20 min; MS (ESIpos): m/z = 497 [M + H]⁺ |
| | 8-bromo-N-[(5,6-dichloro-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: 2.323 (0.59), 2.327 (0.84), | |
| | 2.332 (0.60), 2.518 (3.28), 2.523 (2.06), 2.539 (2.20), 2.665 (0.62), | |
| | 2.669 (0.87), 2.673 (0.62), 3.492 (3.54), 3.602 (4.66), 4.864 (4.00), | |
| | 4.872 (4.00), 7.751 (0.79), 8.040 (16.00), 9.174 (1.37), 12.615 (1.09). | |
| 330 | 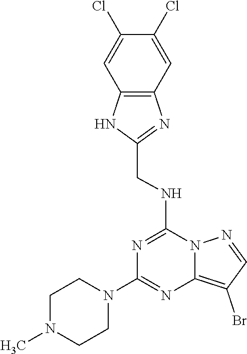 [SMILES: CN1CCN(c2nc(NCc3nc4cc(Cl)c(Cl)cc4[nH]3)n3ncc(Br)c3n2)CC1] | 28% LC-MS (Method 2): Rₜ = 1.17 min; MS (ESIpos): m/z = 510 [M + H]⁺ |
| | 8-bromo-N-[(5,6-dichloro-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: 1.751 (0.25), 2.109 (16.00), | |
| | 2.332 (0.79), 2.336 (0.37), 2.422 (0.32), 2.518 (4.18), 2.523 (2.72), | |
| | 2.539 (3.92), 2.678 (0.32), 3.196 (0.18), 3.565 (1.29), 3.597 (2.24), | |
| | 3.912 (0.39), 4.853 (6.95), 5.758 (5.96), 7.740 (5.63), 8.021 (12.54), | |
| | 8.034 (0.19), 9.131 (0.21). | |
| 331 | 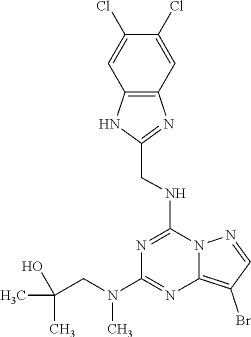 [SMILES: CN(CC(C)(C)O)c1nc(NCc2nc3cc(Cl)c(Cl)cc3[nH]2)n2ncc(Br)c2n1] | 7% LC-MS (Method 1): Rₜ = 1.21 min; MS (ESIpos): m/z = 513 [M + H]⁺ |
| | 1-[(8-bromo-4-{[(5,6-dichloro-1H-benzimidazol-2- | |
| | yl)methyl]amino}pyrazolo[1,5-a][1,3,5]triazin-2- | |
| | yl)(methyl)amino]-2-methylpropan-2-ol | |
| | ¹H-NMR (400 MHz, DMSO-d6) δ [ppm]: 0.738 (16.00) 1.081 (3.17), | |
| | 2.153 (1.46), 2.322 (1.01), 2.327 (1.41), 2.332 (1.04), 2.422 (0.45), | |
| | 2.518 (6.95), 2.522 (4.20), 2.539 (2.42), 2.665 (1.04), 2.669 (1.43), | |
| | 2.673 (1.04), 3.012 (1.63), 3.119 (9.69), 3.396 (4.67), 3.549 (0.99), | |
| | 4.362 (2.84), 4.800 (2.52), 4.813 (2.65), 4.874 (0.89), 7.634 (1.21), | |
| | 7.794 (1.26), 8.007 (14.81), 9.050 (1.29), 12.600 (1.43). | |
| 332 | 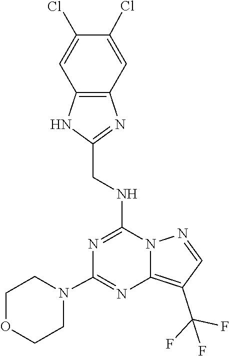 [SMILES: FC(F)(F)c1cnn2c(NCc3nc4cc(Cl)c(Cl)cc4[nH]3)nc(N3CCOCC3)nc12] | 47% LC-MS (Method 2): Rₜ = 1.26 min; MS (ESIpos): m/z = 487 [M + H]⁺ |
| | N-[(5,6-dichloro-1H-benzimidazol-2-yl)methyl]-2-(morpholin-4- | |
| | yl)-8-(trifluoromethyl)pyrazolo[1,5-a][1,3,5]triazin-4-amine | |
| | ¹H-NMR (400 MHz, DMSO-d6) δ [ppm]: 0.817 (0.83), 0.822 (0.50), | |
| | 0.835 (1.00), 0.852 (0.61), 0.884 (0.56), 0.902 (1.11), 0.905 (0.56), | |
| | 0.920 (0.56), 1.009 (0.44), 1.016 (1.00), 1.035 (1.00), 1.232 (1.78), | |
| | 1.988 (0.50), 2.066 (1.33), 2.318 (1.00), 2.323 (2.28), 2.327 (3.33), | |
| | 2.332 (2.33), 2.336 (1.00), 2.518 (11.89), 2.523 (8.11), 2.660 (1.00), | |
| | 2.665 (2.39), 2.669 (3.39), 2.673 (2.39), 2.678 (1.06), 3.494 (2.78), | |
| | 3.618 (4.78), 4.881 (12.78), 7.753 (2.44), 8.262 (16.00), 9.323 (1.00), | |
| | 12.617 (0.78). | |
| 333 | 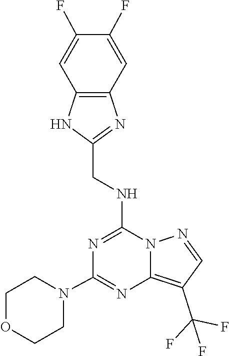 [SMILES: Fc1cc2nc(CNc3nc(N4CCOCC4)nc4c(C(F)(F)F)cnn34)[nH]c2cc1F] | 35% LC-MS (Method 2): Rₜ = 1.17 min; MS (ESIpos): m/z = 455 [M + H]⁺ |
| | N-[(5,6-difluoro-1H-benzimidazol-2-yl)methyl]-2-(morpholin-4- | |
| | yl)-8-(trifluoromethyl)pyrazolo[1,5-a][1,3,5]triazin-4-amine | |
| | ¹H-NMR (400 MHz, DMSO-d6) δ [ppm]: 1.233 (0.68), 2.318 (0.87), | |
| | 2.323 (1.99), 2.327 (2.91), 2.332 (2.08), 2.336 (0.87), 2.518 (10.52), | |
| | 2.523 (6.93), 2.660 (0.87), 2.665 (2.08), 2.669 (2.96), 2.673 (2.13), | |
| | 2.678 (0.92), 3.503 (2.96), 3.628 (5.43), 4.862 (14.98), 7.528 (1.55), | |
| | 8.258 (16.00), 9.299 (0.73), 12.497 (0.68). | |
| 334 | 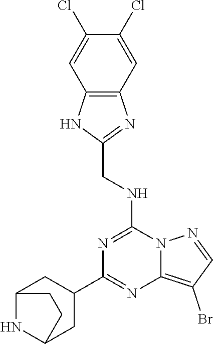 [SMILES: Clc1cc2nc(CNc3nc(C4CC5CCC(C4)N5)nc4c(Br)cnn34)[nH]c2cc1Cl] | 19% LC-MS (Method 2): Rₜ = 1.10 min; MS (ESIpos): m/z = 522 [M + H]⁺ |
| | 8-bromo-2-(3,8-diazabicyclo[3.2.1]octan-3-yl)-N-[(5,6-dichloro- | |
| | 1H-benzimidazol-2-yl)methyl]pyrazolo[1,5-a][1,3,5]triazin-4- | |
| | amine | |
| | ¹H-NMR (400 MHz, DMSO-d6) δ [ppm]: 0.833 (0.65), 0.850 (0.93), | |
| | 0.988 (0.65), 1.078 (0.51), 1.094 (0.53), 1.172 (0.48), 1.232 (3.29), | |
| | 1.352 (6.63), 1.475 (0.72), 1.757 (0.92), 1.899 (0.85), 2.180 (0.78), | |
| | 2.518 (9.20), 2.522 (5.73), 2.539 (1.75), 2.739 (1.28), 2.836 (0.73), | |
| | 3.154 (0.89), 3.590 (0.50), 3.599 (0.82), 3.960 (0.62), 4.180 (0.64), | |
| | 4.810 (5.07), 6.868 (0.47), 7.737 (2.00), 7.998 (16.00), 8.017 (0.58), | |
| | 9.103 (0.53). | |
| 335 | 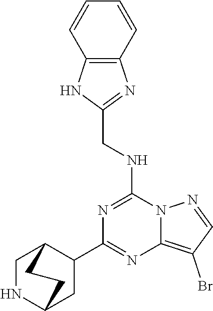 [SMILES: Brc1cnn2c(NCc3nc4ccccc4[nH]3)nc(C3C[C@H]4CC[C@@H]3CN4)nc12] | 25% LC-MS (Method 2): Rₜ = 0.94 min; MS (ESIpos): m/z = 454 [M + H]⁺ |
| | N-[(1H-benzimidazol-2-yl)methyl]-8-bromo-2-{[1R or S- | |
| | (1R*,4R*)]-2,5-diazabicyclo[2.2.2]octan-2-yl}pyrazolo[1,5- | |
| | a][1,3,5]triazin-4-amine (Enantiomer 2) | |
| | ¹H-NMR (400 MHz, DMSO-d6) δ [ppm]: 1.232 (0.31), 1.353 (0.23), | |
| | 1.539 (0.64), 1.732 (0.26), 1.797 (0.80), 1.892 (0.17), 2.539 (16.00), | |
| | 2.764 (1.05), 2.934 (0.35), 2.972 (0.64), 2.994 (0.38), 3.040 (0.28), | |
| | 3.402 (0.59), 3.433 (0.57), 3.471 (0.39), 3.053 (0.82), 3.556 (0.52), | |
| | 3.585 (0.34), 4.389 (0.52), 4.600 (0.35), 4.809 (1.73), 4.843 (0.18), | |
| | 4.902 (0.98), 7.125 (1.16), 7.398 (0.31), 7.527 (0.39), 7.984 (1.95), | |
| | 7.989 (1.38), 12.246 (0.29). | |
| 336 | 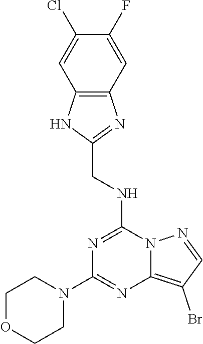 [SMILES: Fc1cc2nc(CNc3nc(N4CCOCC4)nc4c(Br)cnn34)[nH]c2cc1Cl] | 17% LC-MS (Method 1): Rₜ = 1.14 min; MS (ESIpos): m/z = 481 [M + H]⁺ |
| | 8-bromo-N-[(6-chloro-5-fluoro-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: 2.518 (9.49), 2.523 (5.76), | |
| | 2.539 (1.31), 3.494 (3.60), 3.606 (4.70), 3.638 (1.43), 3.705 (0.74), | |
| | 3.716 (0.93), 3.722 (1.02), 3.733 (0.90), 3.746 (0.74), 4.850 (4.17), | |
| | 4.864 (4.19), 7.467 (0.40), 7.600 (0.70), 7.754 (0.42), 7.997 (1.72), | |
| | 8.037 (16.00), 8.202 (0.76), 9.163 (1.76), 12.582 (0.65). | |
| 337 | 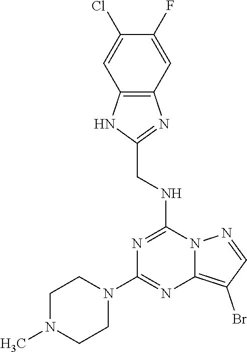 [SMILES: CN1CCN(c2nc(NCc3nc4cc(F)c(Cl)cc4[nH]3)n3ncc(Br)c3n2)CC1] | 19% LC-MS (Method 2): Rₜ = 1.11 min; MS (ESIpos): m/z = 494 [M + H]⁺ |
| | 8-bromo-N-[(6-chloro-5-fluoro-1H-benzimidazol-2-yl)methyl]-2- | |
| | (4-methylpiperazin-1-yl)pyrazolo[1,5-a][1,3,5]triazin-4-amine | |
| | ¹H-NMR (400 MHz, DMSO-d6) δ [ppm]: 1.052 (0.79), 1.070 (0.41), | |
| | 1.178 (1.92), 1.232 (1.18), 1.751 (0.45), 2.155 (7.93), 2.218 (2.40), | |
| | 2.287 (1.13), 2.318 (1.24), 2.322 (2.13), 2.327 (2.74), 2.332 (2.08), | |
| | 2.336 (1.11), 2.518 (8.80), 2.523 (5.48), 2.539 (1.22), 2.555 (0.59), | |
| | 2.660 (0.80), 2.664 (1.68), 2.669 (2.43), 2.673 (1.74), 2.678 (0.79), | |
| | 3.565 (2.01), 3.618 (3.15), 4.843 (5.92), 4.856 (5.90), 5.758 (3.66), | |
| | 7.438 (3.03), 7.462 (3.02), 7.580 (4.88), 7.598 (3.18), 7.604 (2.97), | |
| | 7.747 (2.89), 7.765 (2.86), 8.025 (16.00), 8.039 (0.40), 9.127 (2.07), | |
| | 12.546 (2.04), 12.584 (2.15). | |
| 338 | 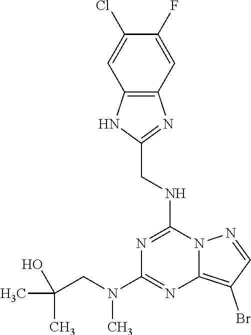 [SMILES: CN(CC(C)(C)O)c1nc(NCc2nc3cc(F)c(Cl)cc3[nH]2)n2ncc(Br)c2n1] | 9% LC-MS (Method 1): Rₜ = 1.14 min; MS (ESIpos): m/z = 497 [M + H]⁺ |
| | 1-[(8-bromo-4-{[(6-chloro-5-fluoro-1H-benzimidazol-2- | |
| | yl)methyl]amino}pyrazolo[1,5-a][1,3,5]triazin-2- | |
| | yl)(methyl)amino]-2-methylpropan-2-ol | |
| | ¹H-NMR (400 MHz, DMSO-d6) δ [ppm]: 0.739 (16.00), 1.083 (3.65), | |
| | 2.101 (0.43), 2.303 (0.46), 2.518 (13.24), 2.522 (8.40), 2.539 (1.73), | |
| | 3.021 (1.75), 3.121 (10.92), 3.404 (4.03), 3.552 (1.17), 4.363 (3.99), | |
| | 4.790 (2.70), 4.802 (2.76), 4.881 (1.45), 7.403 (0.93), 7.426 (1.04), | |
| | 7.547 (1.80), 7.562 (1.24), 7.572 (1.26), 7.712 (0.94), 7.730 (1.02), | |
| | 8.005 (13.84), 8.143 (0.45), 9.038 (1.40), 12.526 (1.18), 12.566 | |
| | (1.32). | |
| 339 | 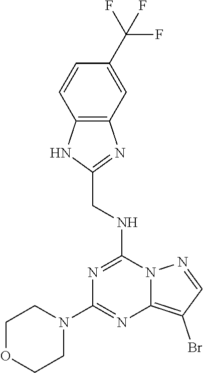 [SMILES: FC(F)(F)c1ccc2[nH]c(CNc3nc(N4CCOCC4)nc4c(Br)cnn34)nc2c1] | 14% LC-MS (Method 1): Rₜ = 1.14 min; MS (ESIpos): m/z = 497 [M + H]⁺ |
| | 8-bromo-2-(morpholin-4-yl)-N-{[5-(trifluoromethyl)-1H- | |
| | benzimidazol-2-yl]methyl}pyrazolo[1,5-a][1,3,5]triazin-4-amine | |
| | ¹H-NMR (400 MHz, DMSO-d6) δ [ppm]: 2.518 (8.93), (5.59), | |
| | 3.468 (2.95), 3.592 (4.37), 3.639 (0.81), 3.722 (0.51), 4.908 (3.93), | |
| | 4.920 (3.98), 7.462 (1.56), 7.483 (1.96), 7.620 (0.47), 7.735 (0.41), | |
| | 7.903 (0.53), 7.997 (0.81), 8.048 (16.00), 9.214 (1.55), 12.728 (1.14). | |
| 340 | 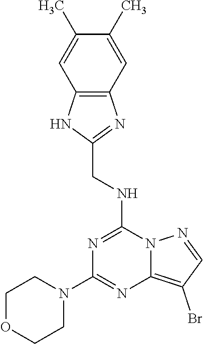 [SMILES: Cc1cc2nc(CNc3nc(N4CCOCC4)nc4c(Br)cnn34)[nH]c2cc1C] | 10% LC-MS (Method 1): Rₜ = 0.92 min; MS (ESIneg): m/z = 455 [M − H]⁻ |
| | 8-bromo-N-[(5,6-dimethyl-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: 2.271 (16.00), 2.518 (3.41), | |
| | 2.522 (2.21), 3.518 (2.10), 3.636 (2.91), 4.821 (2.35), 4.836 (2.37), | |
| | 7.270 (0.29), 7.359 (0.20), 7.378 (0.24), 7.478 (0.60), 7.496 (0.19), | |
| | 7.516 (0.20), 7.519 (0.24), 8.028 (6.63), 8.143 (4.51), 9.066 (0.54), | |
| | 9.081 (1.17), 9.095 (0.53). | |
| 341 | 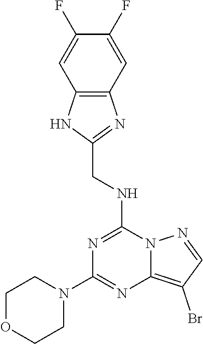 [SMILES: Fc1cc2nc(CNc3nc(N4CCOCC4)nc4c(Br)cnn34)[nH]c2cc1F] | 15% LC-MS (Method 1): Rₜ = 1.04 min; MS (ESIpos): m/z = 465 [M + H]⁺ |
| | 8-bromo-N-[(5,6-difluoro-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: 2.518 (5.30), 2.522 (3.27), | |
| | 3.499 (4.05), 3.612 (5.34), 4.841 (5.37), 4.856 (5.41), 7.319 (0.43), | |
| | 7.458 (0.80), 7.592 (0.75), 8.035 (16.00), 8.159 (2.39), 9.135 (1.20), | |
| | 9.149 (2.51), 9.164 (1.21), 12.503 (1.23). | |
| 342 | 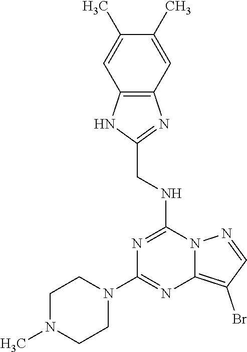 [SMILES: Cc1cc2nc(CNc3nc(N4CCN(C)CC4)nc4c(Br)cnn34)[nH]c2cc1C] | 33% LC-MS (Method 2): Rₜ = 1.11 min; MS (ESIpos): m/z = 470 [M + H]⁺ |
| | 8-bromo-N-[(5,6-dimethyl-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: 1.053 (0.74), 2.128 (16.00), | |
| | 2.200 (2.23), 2.266 (14.12), 2.276 (14.20), 2.332 (0.78), 2.518 (4.27), | |
| | 2.522 (2.63), 3.636 (3.52), 4.815 (3.30), 4.828 (3.33), 5.758 (1.11), | |
| | 7.159 (4.19), 7.294 (4.05), 8.009 (12.84), 9.010 (0.74), 9.024 (1.36), | |
| | 9.037 (0.70), 11.987 (2.57). | |
| 343 | 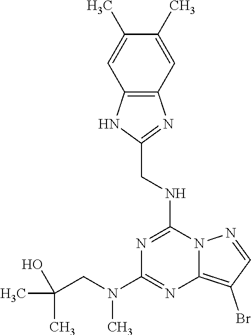 [SMILES: Cc1cc2nc(CNc3nc(N(C)CC(C)(C)O)nc4c(Br)cnn34)[nH]c2cc1C] | 31% LC-MS (Method 1): Rₜ = 0.92 min; MS (ESIpos): m/z = 473 [M + H]⁺ |
| | 1-[(8-bromo-4-{[(5,6-dimethyl-1H-benzimidazol-2- | |
| | yl)methyl]amino}pyrazolo[1,5-a][1,3,5]triazin-2- | |
| | yl)(methyl)amino]-2-methylpropan-2-ol | |
| | ¹H-NMR (400 MHz, DMSO-d6) δ [ppm]: 0.843 (7.43), 1.093 (1.91), | |
| | 1.147 (0.43), 2.268 (16.00), 2.518 (2.68), 2.523 (1.76), 2.539 (1.59), | |
| | 3.064 (0.98), 3.136 (4.53), 3.454 (2.19), 3.561 (0.57), 4.391 (0.57), | |
| | 4.764 (1.23), 4.778 (1.27), 7.151 (0.46), 7.272 (0.49), 7.991 (7.72), | |
| | 8.150 (2.48), 8.946 (0.66), 11.964 (0.41). | |
| 344 | 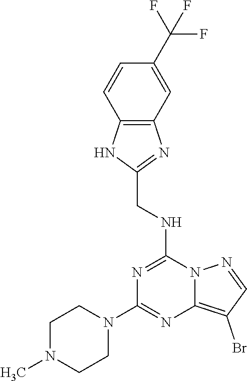 [SMILES: CN1CCN(c2nc(NCc3nc4cc(C(F)(F)F)ccc4[nH]3)n3ncc(Br)c3n2)CC1] | 31% LC-MS (Method 2): Rₜ = 1.17 min; MS (ESIpos): m/z = 510 [M + H]⁺ |
| | 8-bromo-2-(4-methylpiperazin-1-yl)-N-{[5-(trifluoromethyl)-1H- | |
| | benzimidazol-2-yl]methyl}pyrazolo[1,5-a][1,3,5]triazin-4-amine | |
| | ¹H-NMR (400 MHz, DMSO-d6) δ [ppm]: 2.072 (16.00), 2.209 (0.48), | |
| | 2.523 (4.85), 2.539 (1.31), 3.578 (2.17), 4.896 (4.86), 7.459 (1.61), | |
| | 7.479 (2.03), 7.650 (0.57), 7.849 (0.43), 8.029 (11.12), 9.175 (0.74), | |
| | 12.719 (0.44). | |
| 345 | 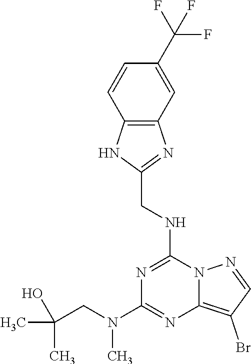 [SMILES: CN(CC(C)(C)O)c1nc(NCc2nc3cc(C(F)(F)F)ccc3[nH]2)n2ncc(Br)c2n1] | 17% LC-MS (Method 1): Rₜ = 1.16 min; MS (ESIpos): m/z = 513 [M + H]⁺ |
| | 1-{[8-bromo-4-({[5-(trifluoromethyl)-1H-benzimidazol-2- | |
| | yl]methyl}amino)pyrazolo[1,5-a][1,3,5]triazin-2- | |
| | yl](methyl)amino}-2-methylpropan-2-ol | |
| | ¹H-NMR (400 MHz, DMSO-d6) δ [ppm]: 0.686 (16.00), 1.080 (3.48), | |
| | 2.518 (4.48), 2.523 (2.93), 2.539 (3.34), 3.012 (1.82), 3.112 (11.89), | |
| | 3.383 (5.58), 3.549 (1.08), 4.333 (2.77), 4.844 (3.01), 4.857 (3.13), | |
| | 4.939 (0.74), 7.459 (1.67), 7.478 (1.67), 7.566 (1.00), 7.586 (0.80), | |
| | 7.705 (1.30), 7.872 (1.44), 8.015 (14.95), 8.155 (0.48), 9.083 (1.24), | |
| | 12.708 (2.14). | |
| 346 | 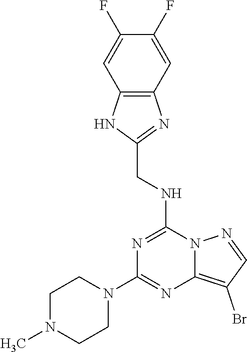 [SMILES: CN1CCN(c2nc(NCc3nc4cc(F)c(F)cc4[nH]3)n3ncc(Br)c3n2)CC1] | 21% LC-MS (Method 2): Rₜ = 1.08 min; MS (ESIpos): m/z = 478 [M + H]⁺ |
| | 8-bromo-N-[(5,6-difluoro-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: 1.231 (0.84), 1.983 (0.18), | |
| | 2.119 (16.00), 2.171 (1.74), 2.206 (2.01), 2.213 (1.83), 2.322 (0.80), | |
| | 2.327 (1.15), 2.331 (1.00), 2.522 (3.67), 2.539 (1.24), 2.665 (0.70), | |
| | 2.669 (0.98), 2.673 (0.72), 3.609 (2.83), 3.723 (0.25), 4.838 (6.49), | |
| | 5.759 (0.20), 7.289 (0.25), 7.293 (0.25), 7.523 (1.15), 7.974 (0.43), | |
| | 8.016 (12.22), 8.030 (0.59), 9.104 (0.36), 12.496 (0.18). | |
| 347 | 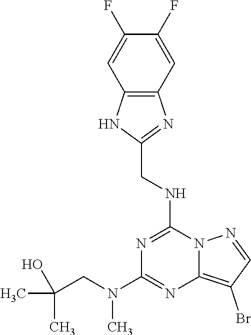 [SMILES: CN(CC(C)(C)O)c1nc(NCc2nc3cc(F)c(F)cc3[nH]2)n2ncc(Br)c2n1] | 14% LC-MS (Method 1): Rₜ = 1.07 min; MS (ESIpos): m/z = 481 [M + H]⁺ |
| | 1-[(8-bromo-4-{[(5,6-difluoro-1H-benzimidazol-2- | |
| | yl)(methyl]amino}pyrazolo[1,5-a][1,3,5]triazin-2- | |
| | yl)(methyl)amino]-2-methylpropan-2-ol | |
| | ¹H-NMR (400 MHz, DMSO-d6) δ [ppm]: 0.745 (14.64), 1.086 (2.85), | |
| | 1.105 (1.85), 1.151 (1.00), 2.075 (0.41), 2.518 (4.97), 2.523 (3.21), | |
| | 2.539 (1.30), 3.011 (0.65), 3.034 (1.50), 3.124 (8.97), 3.417 (4.28), | |
| | 3.555 (0.89), 4.366 (2.52), 4.780 (2.30), 4.793 (2.38), 4.885 (1.00), | |
| | 7.393 (0.54), 7.413 (0.86), 7.437 (0.69), 7.542 (0.51), 7.568 (0.75), | |
| | 7.587 (0.65), 8.002 (16.00), 8.015 (0.46), 8.189 (0.53), 9.025 (1.14), | |
| | 12.488 (1.43). | |
| 348 | 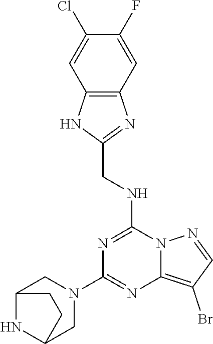 [SMILES: Fc1cc2nc(CNc3nc(N4CC5CCC(C4)N5)nc4c(Br)cnn34)[nH]c2cc1Cl] | 11% LC-MS (Method 2): Rₜ = 1.06 min; MS (ESIneg): m/z = 504 [M − H]⁻ |
| | 8-bromo-N-[(6-chloro-5-fluoro-1H-benzimidazol-2-yl)methyl]-2- | |
| | (3,8-diazabicyclo[3.2.1]octan-3-yl)pyrazolo[1,5-a][1,3,5]triazin-4-amine | |
| | ¹H-NMR (500 MHz, DMSO-d6) δ [ppm]: 1.002 (0.62), 1.038 (7.11), | |
| | 1.052 (12.94), 1.067 (6.95), 1.258 (0.68), 1.353 (0.81), 1.483 (0.61), | |
| | 1.751 (1.62), 2.072 (0.42), 2.514 (4.62), 2.518 (4.15), 2.522 (3.28), | |
| | 2.743 (0.76), 2.836 (0.74), 3.154 (0.87), 3.411 (0.65), 3.425 (1.68), | |
| | 3.435 (1.76), 3.439 (1.70), 3.449 (1.72), 3.462 (0.65), 3.967 (2.58), | |
| | 4.180 (0.68), 4.346 (0.83), 4.356 (1.50), 4.366 (0.78), 4.801 (4.97), | |
| | 7.507 (0.61), 7.684 (0.70), 7.699 (0.61), 7.995 (16.00), 9.084 (0.45). | |
| 349 | 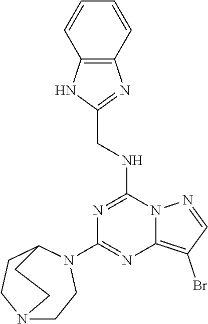 [SMILES: Brc1cnn2c(NCc3nc4ccccc4[nH]3)nc(N3CCN4CCC3CC4)nc12] | 36% LC-MS (Method 2): Rₜ = 1.01 min; MS (ESIpos): m/z = 468 [M + H]⁺ |
| | N-[(1H-benzimidazol-2-yl)methyl]-8-bromo-2-(1,4- | |
| | diazabicyclo[3.2.2]nonan-4-yl)pyrazolo[1,5-a][1,3,5]triazin-4-amine | |
| | ¹H-NMR (400 MHz, DMSO-d6) δ [ppm]: 1.231 (2.03), 1.365 (2.07), | |
| | 1.605 (1.55), 1.693 (2.13), 1.951 (1.55), 2.074 (4.41), 2.291 (0.52), | |
| | 2.323 (1.45), 2.327 (2.02), 2.331 (1.43), 2.523 (7.00), 2.577 (2.37), | |
| | 2.665 (1.97), 2.669 (2.66), 2.674 (2.42), 2.725 (4.53), 2.827 (5.13), | |
| | 3.159 (0.52), 3.821 (2.35), 3.949 (3.36), 4.425 (1.87), 4.617 (1.34), | |
| | 4.833 (10.09), 4.845 (10.14), 7.125 (7.00), 7.388 (2.98), 7.516 (3.54), | |
| | 7.530 (3.18), 7.990 (16.00), 8.431 (0.61), 9.018 (2.30), 12.242 (3.46). | |
| 350 | 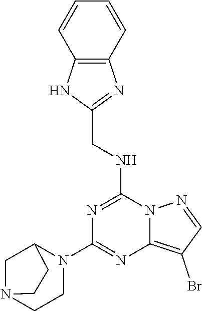 [SMILES: Brc1cnn2c(NCc3nc4ccccc4[nH]3)nc(N3CCN4CCC3C4)nc12] | 5% LC-MS (Method 2): Rₜ = 0.95 min; MS (ESIpos): m/z = 454 [M + H]⁺ |
| | N-[(1H-benzimidazol-2-yl)methyl]-8-bromo-2-(1,4- | |
| | diazabicyclo[3.2.1]octan-4-yl)pyrazolo[1,5-a][1,3,5]triazin-4-amine | |
| | ¹H-NMR (400 MHz, DMSO-d6) δ [ppm]: 0.850 (0.67), 1.231 (3.27), | |
| | 1.586 (0.62), 1.752 (1.64), 2.288 (0.43), 2.322 (1.77), 2.326 (2.26), | |
| | 2.331 (1.70), 2.522 (9.34), 2.539 (5.58), 2.669 (3.38), 2.673 (2.81), | |
| | 2.712 (1.67), 2.847 (1.29), 2.971 (0.81), 2.993 (1.22), 3.992 (1.26), | |
| | 4.004 (1.34), 4.025 (1.29), 4.815 (0.48), 4.857 (10.21), 4.900 (0.58), | |
| | 7.114 (6.16), 7.122 (6.28), 7.129 (6.45), 7.137 (6.80), 7.466 (3.00), | |
| | 8.000 (16.00), 8.009 (1.28), 8.026 (0.49), 8.549 (0.64). | |
| 351 | 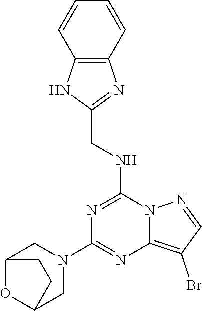 [SMILES: Brc1cnn2c(NCc3nc4ccccc4[nH]3)nc(N3CC4CCC(C3)O4)nc12] | 55% LC-MS (Method 2): Rₜ = 1.06 min; MS (ESIpos): m/z = 455 [M + H]⁺ |
| | N-[(1H-benzimidazol-2-yl)methyl]-8-bromo-2-(8-oxa-3- | |
| | azabicyclo[3.2.1]octan-3-yl)pyrazolo[1,5-a][1,3,5]triazin-4-amine | |
| | ¹H-NMR (400 MHz, DMSO-d6) δ [ppm]: 1.230 (1.52), 1.498 (1.41), | |
| | 1.648 (0.82), 2.074 (2.31), 2.948 (1.90), 3.165 (1.25), 4.149 (1.91), | |
| | 4.835 (13.96), 7.102 (1.37), 7.112 (7.14), 7.120 (6.97), 7.127 (7.26), | |
| | 7.134 (7.98), 7.144 (1.66), 7.460 (4.22), 8.019 (16.00). | |
| 352 | 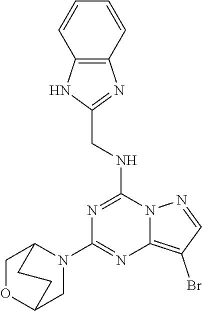 [SMILES: Brc1cnn2c(NCc3nc4ccccc4[nH]3)nc(N3CC4CCC3CO4)nc12] | 19% LC-MS (Method 2): Rₜ = 1.05 min; MS (ESIpos): m/z = 455 [M + H]⁺ |
| | N-[(1H-benzimidazol-2-yl)methyl]-8-bromo-2-(2-oxa-5- | |
| | azabicyclo[2.2.2]octan-5-yl)pyrazolo[1,5-a][1,3,5]triazin-4-amine | |
| | ¹H-NMR (400 MHz, DMSO-d6) δ [ppm]: 1.232 (1.04), 1.548 (2.32), | |
| | 1.575 (3.25), 1.614 (1.72), 1.902 (2.56), 2.074 (0.79), 2.523 (8.85), | |
| | 3.472 (1.12), 3.501 (1.59), 3.561 (1.78), 3.590 (2.75), 3.635 (1.17), | |
| | 3.697 (6.31), 3.896 (1.91), 3.926 (6.25), 4.428 (2.93), 4.629 (1.82), | |
| | 4.816 (4.44), 4.828 (4.53), 4.901 (2.73), 4.913 (2.58), 7.122 (5.10), | |
| | 7.127 (5.04), 7.376 (2.12), 7.398 (2.21), 7.422 (1.22), 7.515 (2.20), | |
| | 7.532 (3.14), 8.013 (16.00), 9.086 (2.75), 12.231 (2.90), 12.257 (1.83). | |
| 353 | 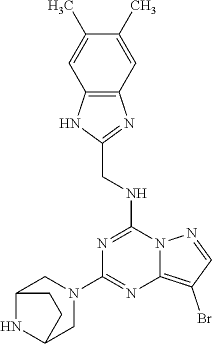 [SMILES: Cc1cc2nc(CNc3nc(N4CC5CCC(C4)N5)nc4c(Br)cnn34)[nH]c2cc1C] | 37% LC-MS (Method 2): Rₜ = 1.08 min; MS (ESIpos): m/z = 482 [M + H]⁺ |
| | 8-bromo-2-(3,8-diazabicyclo[3.2.1]octan-3-yl)-N-[(5,6-dimethyl- | |
| | 1H-benzimidazol-2-yl)methyl]pyrazolo[1,5-a][1,3,5]triazin-4-amine | |
| | ¹H-NMR (400 MHz, DMSO-d6) δ [ppm]: 1.232 (0.73), 1.420 (0.87), | |
| | 1.529 (0.49), 2.074 (0.50), 2.270 (16.00), 2.539 (1.83), 2.634 (0.52), | |
| | 2.851 (0.71), 4.120 (0.50), 4.217 (0.53), 4.790 (4.69), 5.063 (1.09), 7.153 | |
| | (1.74), 7.291 (1.82), 7.356 (1.02), 7.370 (0.54), 7.993 (7.26), 11.970 (1.29). | |
| 354 | 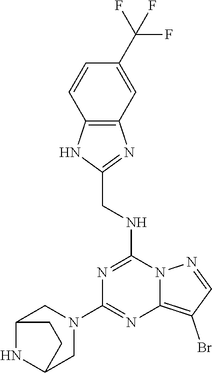 [SMILES: FC(F)(F)c1ccc2[nH]c(CNc3nc(N4CC5CCC(C4)N5)nc4c(Br)cnn34)nc2c1] | 34% LC-MS (Method 2): Rₜ = 1.09 min; MS (ESIpos): m/z = 522 [M + H]hu + |
| | 8-bromo-2-(3,8-diazabicyclo[3.2.1]octan-3-yl)-N-{[5- | |
| | (trifluoromethyl)-1H-benzimidazol-2-yl]methyl}pyrazolo[1,5- | |
| | a][1,3,5]triazin-4-amine | |
| | ¹H-NMR (400 MHz, DMSO-d6) δ [ppm]: 1.035 (1.50), 1.052 (2.72), 1.070 | |
| | (1.70), 1.150 (0.90), 1.166 (0.89), 1.232 (1.03), 1.437 (0.84), 1.626 (0.59), | |
| | 1.751 (1.01), 2.518 (8.15), 2.523 (5.07), 2.540 (5.11), 2.563 (2.08), 2.994 | |
| | (0.83), 3.385 (3.68), 3.427 (1.14), 3.446 (1.16), 3.462 (1.15), 3.478 (1.16), | |
| | 3.573 (1.65), 3.590 (0.93), 3.651 (0.58), 3.656 (0.58), 3.665 (0.71), 3.682 | |
| | (0.87), 3.701 (0.92), 3.831 (1.25), 4.106 (0.58), 4.308 (0.58), 4.627 (0.70), | |
| | 4.879 (4.17), 7.012 (0.41), 7.457 (1.82), 7.479 (2.16), 7.592 (0.79), 7.637 | |
| | (0.49), 7.736 (0.88), 7.913 (0.99), 7.999 (0.51), 8.011 (0.67), 8.052 (16.00), | |
| | 8.170 (1.09), 9.266 (0.55), 12.738 (0.68). | |
| 355 | 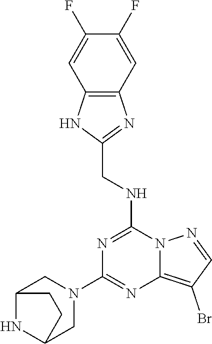 [SMILES: Fc1cc2nc(CNc3nc(N4CC5CCC(C4)N5)nc4c(Br)cnn34)[nH]c2cc1F] | 20% LC-MS (Method 2): Rₜ = 1.00 min; MS (ESIpos): m/z = 490 [M + H]⁺ |
| | 8-bromo-2-(3,8-diazabicyclo[3.2.1]octan-3-yl)-N-[(5,6-difluoro- | |
| | 1H-benzimidazol-2-yl)methyl]pyrazolo[1,5-a][1,3,5]triazin-4-amine | |
| | ¹H-NMR (400 MHz, DMSO-d6) δ [ppm]: 1.035 (1.40), 1.052 (2.48), 1.070 | |
| | (1.18), 1.233 (0.59), 1.282 (1.33), 1.353 (0.72), 1.492 (0.49), 2.518 (16.00), | |
| | 2.523 (11.27), 2.540 (3.84), 2.778 (0.60), 2.851 (0.60), 3.216 (0.75), 4.005 | |
| | (0.55), 4.794 (3.77), 7.407 (0.43), 7.447 (0.66), 7.595 (0.62), 7.999 (12.20), | |
| | 9.091 (0.56), 12.501 (0.61). | |
| 356 | 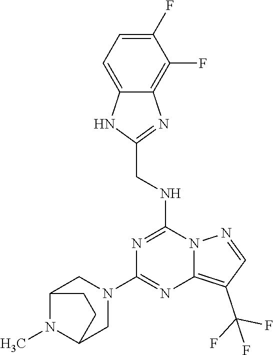 [SMILES: CN1C2CCC1CN(c1nc(NCc3nc4c(F)c(F)ccc4[nH]3)n3ncc(C(F)(F)F)c3n1)C2] | 53% LC-MS (Method 2): Rₜ = 1.16 min; MS (ESIpos): m/z = 494 [M + H]⁺ |
| | N-[(4,5-difluoro-1H-benzimidazol-2-yl)methyl]-2-(8-methyl-3,8- | |
| | diazabicyclo[3.2.1]octan-3-yl)-8-(trifluoromethyl)pyrazolo[1,5- | |
| | a][1,3,5]triazin-4-amine | |
| | ¹H-NMR (400 MHz, DMSO-d6) δ [ppm]: −0.184 (0.53), 0.922 (0.63), 1.232 | |
| | (1.16), 1.244 (0.98), 1.261 (1.29), (0.70), 1.546 (0.56), 1.772 (0.53), | |
| | 1.987 (0.45), 2.106 (16.00), 2.117 (1.45), 2.336 (0.45), 2.518 (5.27), 2.522 | |
| | (3.45), 2.678 (0.45), 2.821 (0.88), 2.852 (1.64), 2.921 (0.88), 2.952 (0.84), | |
| | 3.080 (0.89), 3.965 (0.68), 3.995 (0.67), 4.166 (0.96), 4.184 (0.48), 4.201 | |
| | (0.77), 4.828 (2.98), 4.841 (2.94), 7.176 (1.00), 7.192 (1.64), 7.203 (1.76), | |
| | 7.212 (1.68), 8.216 (0.46), 8.230 (5.91), 9.297 (0.79), 12.668 (0.82). | |
| 357 | 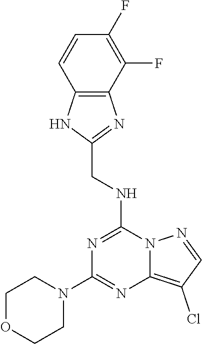 [SMILES: Fc1ccc2[nH]c(CNc3nc(N4CCOCC4)nc4c(Cl)cnn34)nc2c1F] | 40% LC-MS (Method 1): Rₜ = 1.06 min; MS (ESIpos): m/z = 421 [M + H]⁺ |
| | 8-chloro-N-[4,5-difluoro-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: 2.518 (4.49), 2.523 (2.81), 2.539 | |
| | (4.24), 3.292 (0.50), 3.380 (0.84), 3.388 (0.53), 3.489 (4.77), 3.609 (6.89), | |
| | 4.867 (6.72), 4.881 (6.65), 7.159 (0.63), 7.181 (1.48), 7.197 (2.03), 7.208 | |
| | (3.39), 7.221 (3.68), 8.056 (16.00), 9.209 (1.64), 12.690 (1.68). | |
| 358 | 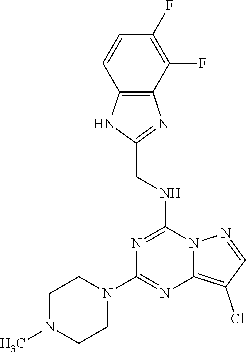 [SMILES: CN1CCN(c2nc(NCc3nc4c(F)c(F)ccc4[nH]3)n3ncc(Cl)c3n2)CC1] | 31% LC-MS (Method 1): Rₜ = 0.75 min; MS (ESIneg): m/z = 432 [M − H]⁻ |
| | 8-chloro-N-[(4,5-difluoro-1H-benzimidazol-2-yl)methyl]-2-(4- | |
| | methylpiperazin-1-yl)pyrazolo[1,5-a][1,3,5]triazin-4-amine | |
| | ¹H-NMR (400 MHz, DMSO-d6) δ [ppm]: 2.115 (16.00), 2.116 (1.40), 2.214 | |
| | (0.71), 2.518 (3.29), 2.523 (2.01), 2.539 (0.99), 3.406 (2.38), 3.606 (2.80), | |
| | 4.854 (3.78), 4.868 (3.80), 7.155 (0.41), 7.177 (1.05), 7.194 (1.20), 7.205 | |
| | (1.52), 7.219 (1.32), 8.038 (11.51), 8.151 (6.01), 9.160 (0.93), 12.694 (0.48). | |
| 359 | 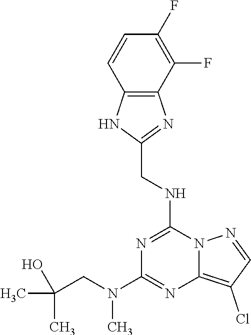 [SMILES: CN(CC(C)(C)O)c1nc(NCc2nc3c(F)c(F)ccc3[nH]2)n2ncc(Cl)c2n1] | 18% LC-MS (Method 1): Rₜ = 1.07 min; MS (ESIpos): m/z = 437 [M + H]⁺ |
| | 1-[(8-chloro-4-{[(4,5-difluoro-1H-benzimidazol-2- | |
| | yl)methyl]amino}pyrazolo[1,5-a][1,3,5]triazin-2- | |
| | yl)(methyl)amino]-2-methylpropan-2-ol | |
| | ¹H-NMR (400 MHz, DMSO-d6) δ [ppm]: 0.702 (16.00), 1.082 (3.40), | |
| | 1.104 (0.91), 2.332 (1.11), 2.522 (4.57), 2.539 (9.95), 2.673 (1.08), | |
| | 3.026 (1.79), 3.112 (13.22), 3.388 (5.64), 3.554 (1.08), 4.322 (3.00), | |
| | 4.798 (3.62), 4.811 (3.74), 4.847 (0.77), 4.898 (0.79), 7.172 (3.06), | |
| | 7.185 (3.31), 8.024 (10.21), 9.081 (1.42), 12.663 (1.69). | |
| 360 | 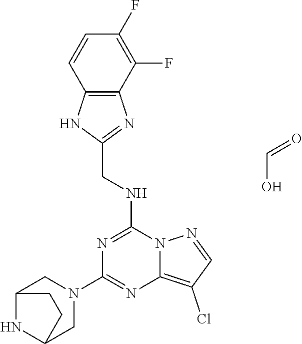 [SMILES: Fc1ccc2[nH]c(CNc3nc(N4CC5CCC(C4)N5)nc4c(Cl)cnn34)nc2c1F ; O=CO] | 27% LC-MS (Method 1): Rₜ = 0.77 min; MS (ESIneg): m/z = 444 [M − H]⁻ |
| | formic acid 8-chloro-2-(3,8-diazabicyclo[3.2.1]octan-3-yl)-N- | |
| | [(4,5-difluoro-1H-benzimidazol-2-yl)methyl]pyrazolo[1,5- | |
| | a][1,3,5]triazin-4-amine | |
| | ¹H-NMR (400 MHz, DMSO-d6) δ [ppm]: 1.048 (0.42), 1.377 (0.65), | |
| | 1.531 (0.43), 2.518 (3.50), 2.523 (2.35), 2.539 (4.16), 2.838 (0.60), | |
| | 2.903 (0.62), 3.282 (1.08), 3.401 (1.40), 4.063 (0.54), 4.217 (0.52), | |
| | 4.824 (5.15), 7.148 (0.68), 7.171 (1.33), 7.177 (0.81), 7.188 (1.43), | |
| | 7.199 (1.48), 7.216 (1.73), 7.232 (1.08), 8.030 (16.00), 8.253 (10.14). | |
| 361 | 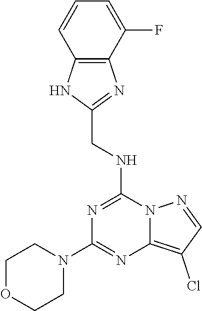 [SMILES: Fc1cccc2[nH]c(CNc3nc(N4CCOCC4)nc4c(Cl)cnn34)nc12] | 27% LC-MS (Method 1): Rₜ = 0.98 min; MS (ESIpos): m/z = 403 [M + H]⁺ |
| | 8-chloro-N-[(4-fluoro-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: 2.518 (4.09), 2.523 (2.79), | |
| | 2.539 (1.52), 3.485 (3.94), 3.615 (6.00), 4.873 (5.96), 4.888 (6.04), | |
| | 6.915 (1.65), 6.917 (1.71), 6.936 (2.14), 6.937 (2.06), 6.943 (1.74), | |
| | 6.945 (1.76), 6.963 (2.06), 6.965 (1.99), 7.007 (0.49), 7.034 (0.47), | |
| | 7.100 (1.62), 7.107 (0.74), 7.113 (1.50), 7.121 (3.35), 7.133 (2.89), | |
| | 7.141 (1.91), 7.153 (1.61), 7.240 (4.54), 7.258 (3.36), 7.380 (0.81), | |
| | 7.399 (0.69), 7.900 (0.50), 8.049 (2.59), 8.057 (16.00), 8.135 (1.04), | |
| | 9.138 (0.56), 9.181 (1.24), 9.196 (2.66), 9.210 (1.21), 12.581 (2.30), | |
| | 12.893 (0.71). | |
| 362 | 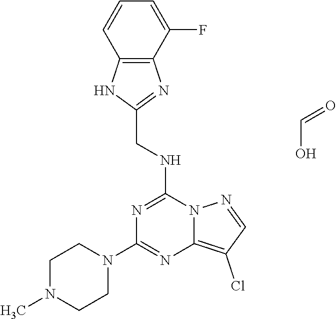 [SMILES: CN1CCN(c2nc(NCc3nc4c(F)cccc4[nH]3)n3ncc(Cl)c3n2)CC1 ; O=CO] | 27% LC-MS (Method 1): Rₜ = 0.69 min; MS (ESIneg): m/z = 414 [M − H]⁻ |
| | formic acid 8-chloro-N-[(4-fluoro-1H-benzimidazol-2-yl)methyl]- | |
| | 2-(4-methylpiperazin-1-yl)pyrazolo[1,5-a][1,3,5]triazin-4-amine | |
| | ¹H-NMR (400 MHz, DMSO-d6) δ [ppm]: 2.074 (1.41), 2.114 (16.00), | |
| | 2.173 (1.67), 2.518 (6.39), 2.522 (4.37), 2.539 (1.81), 3.406 (1.83), | |
| | 3.614 (3.26), 4.861 (4.27), 4.875 (4.32), 6.941 (0.76), 6.965 (0.64), | |
| | 7.095 (0.88), 7.107 (1.04), 7.115 (1.78), 7.127 (1.88), 7.135 (1.12), | |
| | 7.147 (1.01), 7.237 (0.88), 7.255 (0.74), 8.037 (10.84), 8.147 (5.64), | |
| | 9.149 (0.95), 12.583 (0.88). | |
| 363 | 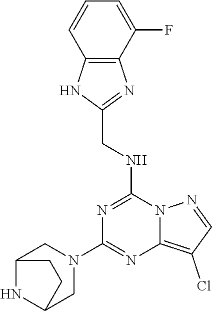 [SMILES: Fc1cccc2[nH]c(CNc3nc(N4CC5CCC(C4)N5)nc4c(Cl)cnn34)nc12] | 24% LC-MS (Method 2): Rₜ = 0.95 min; MS (ESIpos): m/z = 428 [M + H]⁺ |
| | 8-chloro-2-(3,8-diazabicyclo[3.2.1]octan-3-yl)-N-[(4-fluoro-1H- | |
| | benzimidazol-2-yl)methyl]pyrazolo[1,5-a][1,3,5]triazin-4-amine | |
| | ¹H-NMR (400 MHz, DMSO-d6) δ [ppm]: 1.049 (0.66), 1.172 (0.50), | |
| | 1.232 (0.78), 1.333 (0.95), 1.352 (0.97), 1.395 (0.92), 1.416 (0.63), | |
| | 1.488 (0.76), 1.751 (1.59), 1.902 (0.59), 1.988 (0.68), 2.084 (0.72), | |
| | 2.332 (1.09), 2.518 (6.80), 2.523 (4.17), 2.539 (3.20), 2.673 (1.13), | |
| | 2.788 (0.91), 2.855 (0.93), 3.169 (1.23), 3.590 (0.60), 4.017 (0.83), | |
| | 4.035 (0.88), 4.183 (0.86), 4.827 (9.47), 5.758 (3.35), 6.936 (1.15), | |
| | 6.959 (0.94), 7.088 (1.40), 7.100 (1.64), 7.107 (2.88), 7.120 (2.98), | |
| | 7.128 (1.76), 7.140 (1.64), 7.231 (1.19), 7.248 (1.08), 8.017 (16.00), | |
| | 12.574 (0.59). | |
| 364 | 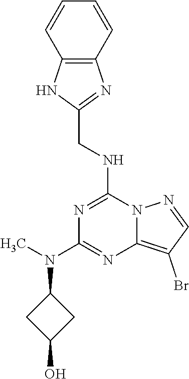 [SMILES: CN(c1nc(NCc2nc3ccccc3[nH]2)n2ncc(Br)c2n1)[C@H]1C[C@@H](O)C1] | 52% LC-MS (Method 2): Rₜ = 0.97 min; MS (ESIpos): m/z = 443 [M + H]⁺ |
| | cis-3-[(4-{[(1H-benzimidazol-2-yl)methyl]amino}-8- | |
| | bromopyrazolo[1,5-a][1,3,5]triazin-2- | |
| | yl)(methyl)amino]cyclobutan-1-ol | |
| | ¹H-NMR (500 MHz, DMSO-d6) δ [ppm]: 1.908 (0.52), 1.913 (0.40), | |
| | 1.923 (0.74), 1.927 (0.90), 1.931 (0.83), 1.942 (0.81), 1.946 (0.96), | |
| | 1.950 (0.80), 1.960 (0.44), 1.965 (0.60), 2.365 (0.76), 2.381 (0.69), | |
| | 2.513 (0.44), 3.017 (16.00), 3.038 (0.73), 3.057 (0.42), 3.776 (0.56), | |
| | 3.804 (0.54), 4.416 (0.58), 4.904 (9.42), 7.125 (3.67), 7.131 (3.26), | |
| | 7.137 (3.41), 7.143 (4.17), 7.151 (0.47), 7.476 (1.66), 7.482 (1.47), | |
| | 7.487 (1.43), 7.494 (1.50), 7.924 (7.60), 8.233 (12.74). | |
| 365 | 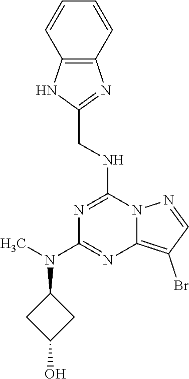 [SMILES: CN(c1nc(NCc2nc3ccccc3[nH]2)n2ncc(Br)c2n1)[C@H]1C[C@H](O)C1] | 75% LC-MS (Method 2): Rₜ = 1.01 min; MS (ESIpos): m/z = 443 [M + H]⁺ |
| | trans-3-[(4-{[(1H-benzimidazol-2-yl)methyl]amino}-8- | |
| | bromopyrazolo[1,5-a][1,3,5]triazin-2- | |
| | yl)(methyl)amino]cyclobutan-1-ol | |
| | ¹H-NMR (600 MHz, DMSO-d6) δ [ppm]: 1.753 (0.57), 2.019 (1.85), | |
| | 2.070 (3.36), 2.307 (1.17), 2.313 (0.89), 2.320 (2.18), 2.324 (1.95), | |
| | 2.332 (1.95), 2.337 (1.89), 2.342 (1.97), 2.349 (0.84), 2.355 (1.05), | |
| | 2.515 (2.83), 2.518 (2.83), 2.521 (2.35), 2.539 (0.59), 2.983 (4.02), | |
| | 4.167 (0.91), 4.179 (1.65), 4.189 (0.91), 4.891 (11.83), 7.105 (1.06), | |
| | 7.112 (7.15), 7.117 (6.51), 7.122 (6.14), 7.128 (7.53), 7.134 (1.20), | |
| | 7.461 (2.91), 7.466 (3.06), 7.469 (3.02), 7.475 (2.69), 7.984 (16.00), | |
| | 8.309 (12.51). | |
| 366 | 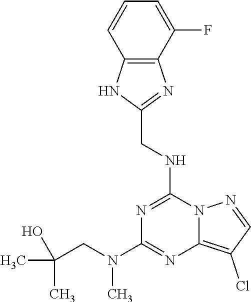 [SMILES: CN(CC(C)(C)O)c1nc(NCc2nc3c(F)cccc3[nH]2)n2ncc(Cl)c2n1] | 12% LC-MS (Method 1): Rₜ = 1.01 min; MS (ESIpos): m/z = 419 [M + H]⁺ |
| | 1-[(8-chloro-4-{[(4-fluoro-1H-benzimidazol-2- | |
| | yl)methyl]amino}pyrazolo[1,5-a][1,3,5]triazin-2- | |
| | yl)(methyl)amino]-2-methylpropan-2-ol | |
| | ¹H-NMR (400 MHz, DMSO-d6) δ [ppm]: 0.689 (2.62), 0.723 (15.91), | |
| | 1.085 (3.52), 1.232 (0.60), 2.318 (1.18), 2.518 (16.00), 2.523 (10.68), | |
| | 2.539 (0.75), 2.678 (1.15), 3.041 (1.82), 3.116 (12.04), 3.403 (5.17), | |
| | 3.558 (1.12), 4.321 (2.92), 4.805 (3.44), 4.819 (3.66), 4.857 (0.76), | |
| | 4.903 (0.82), 6.899 (0.87), 6.920 (1.31), 6.946 (1.25), 7.100 (1.60), | |
| | 7.112 (1.76), 7.132 (1.03), 7.202 (2.03), 7.222 (1.56), 7.515 (1.76), | |
| | 7.535 (3.85), 7.555 (2.38), 7.682 (1.43), 7.702 (1.16), 7.875 (1.16), | |
| | 7.879 (2.77), 7.882 (2.29), 7.887 (3.08), 7.891 (4.17), 7.896 (5.19), | |
| | 8.022 (11.86), 8.142 (2.52), 9.063 (1.50), 12.557 (1.78). | |
| 367 | 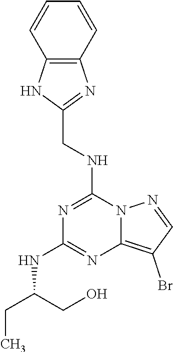 [SMILES: CC[C@@H](CO)Nc1nc(NCc2nc3ccccc3[nH]2)n2ncc(Br)c2n1] | 42% LC-MS (Method 2): Rₜ = 0.92 min; MS (ESIpos): m/z = 431 [M + H]⁺ |
| | (2S)-2-[(4-{[(1H-benzimidazol-2-yl)methyl]amino}-8- | |
| | bromopyrazolo[1,5-a][1,3,5]triazin-2-yl)amino]butan-1-ol | |
| | ¹H-NMR (400 MHz, DMSO-d6) δ [ppm]: 0.579 (1.03), 0.597 (2.07), | |
| | 0.615 (1.14), 0.834 (2.39), 0.852 (5.09), 0.870 (2.84), 1.232 (1.36), | |
| | 1.367 (0.52), 1.386 (0.87), 1.405 (0.94), 1.420 (0.93), 1.439 (0.58), | |
| | 1.606 (0.45), 1.620 (0.60), 1.639 (0.69), 1.655 (0.51), 1.751 (0.42), | |
| | 1.905 (0.49), 2.332 (0.78), 2.518 (3.60), 2.523 (2.35), 2.539 (0.61), | |
| | 2.673 (0.75), 3.265 (0.73), 3.431 (1.14), 3.873 (0.72), 3.886 (0.69), | |
| | 4.641 (0.84), 4.863 (3.88), 6.955 (1.32), 6.977 (1.30), 7.064 (0.63), | |
| | 7.084 (0.67), 7.135 (2.85), 7.401 (1.64), 7.418 (1.31), 7.534 (1.63), | |
| | 7.551 (1.19), 7.967 (16.00), 8.862 (0.88), 12.195 (0.67), 12.264 | |
| | (1.29). | |
| 368 | 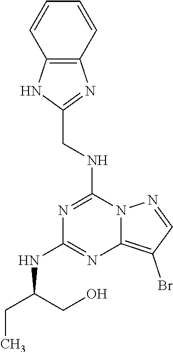 [SMILES: CC[C@H](CO)Nc1nc(NCc2nc3ccccc3[nH]2)n2ncc(Br)c2n1] | 48% LC-MS (Method 2): Rₜ = 0.97 min; MS (ESIpos): m/z = 431 [M + H]⁺ |
| | (2R)-2-[(4-{[(1H-benzimidazol-2-yl)methyl]amino}-8- | |
| | bromopyrazolo[1,5-a][1,3,5]triazin-2-yl)amino]butan-1-ol | |
| | ¹H-NMR (400 MHz, DMSO-d6) δ [ppm]: 0.608 (3.03), 0.625 (1.79), | |
| | 0.836 (3.81), 0.854 (7.67), 0.871 (4.68), 1.230 (1.74), 1.388 (1.41), | |
| | 1.404 (1.66), 1.421 (1.60), 1.609 (1.10), 1.622 (1.06), 1.639 (1.15), | |
| | 1.657 (0.88), 1.751 (1.46), 2.074 (0.77), 2.332 (1.59), 2.518 (8.84), | |
| | 2.522 (5.36), 2.539 (3.03), 2.994 (0.49), 3.427 (2.83), 3.438 (2.80), | |
| | 3.464 (1.64), 3.716 (0.72), 3.872 (1.29), 4.820 (0.58), 4.861 (14.33), | |
| | 4.907 (0.48), 6.957 (1.96), 6.979 (1.96), 7.052 (0.92), 7.073 (0.86), | |
| | 7.116 (5.86), 7.123 (6.15), 7.129 (6.00), 7.469 (5.49), 7.906 (0.52), | |
| | 7.962 (16.00), 8.558 (1.08). | |
| 369 | 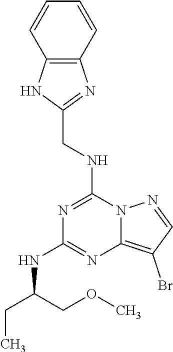 [SMILES: CC[C@H](COC)Nc1nc(NCc2nc3ccccc3[nH]2)n2ncc(Br)c2n1] | 38% LC-MS (Method 2): Rₜ = 1.08 min; MS (ESIpos): m/z = 445 [M + H]⁺ |
| | N4-[(1H-benzimidazol-2-yl)methyl]-8-bromo-N2-[(2R)-1- | |
| | methoxybutan-2-yl]pyrazolo[1,5-a][1,3,5]triazine-2,4-diamine | |
| | ¹H-NMR (400 MHz, DMSO-d6) δ [ppm]: 0.594 (2.06), 0.613 (4.21), | |
| | 0.631 (2.24), 0.836 (3.34), 0.854 (7.11), 0.872 (3.76), 1.197 (0.56), | |
| | 1.213 (0.71), 1.232 (1.62), 1.333 (0.66), 1.348 (0.74), 1.363 (0.65), | |
| | 1.383 (0.78), 1.403 (0.88), 1.420 (0.98), 1.437 (1.02), 1.457 (0.65), | |
| | 1.550 (0.65), 1.565 (0.86), 1.583 (0.96), 1.598 (0.65), 1.616 (0.43), | |
| | 2.518 (5.75), 2.523 (3.59), 2.534 (0.63), 2.538 (0.51), 2.917 (9.11), | |
| | 2.990 (0.83), 3.014 (1.01), 3.029 (0.92), 3.091 (0.92), 3.105 (1.03), | |
| | 3.130 (0.67), 3.238 (16.00), 3.263 (1.34), 3.277 (1.54), 3.286 (2.12), | |
| | 3.301 (2.67), 3.363 (2.50), 3.373 (1.84), 3.388 (1.20), 3.784 (0.68), | |
| | 3.798 (0.68), 4.047 (1.01), 4.059 (0.99), 4.844 (2.08), 4.865 (3.82), | |
| | 4.879 (3.41), 7.088 (2.26), 7.110 (4.28), 7.119 (4.17), 7.194 (1.29), | |
| | 7.215 (1.21), 7.371 (1.14), 7.399 (2.09), 7.417 (1.55), 7.512 (1.23), | |
| | 7.532 (2.27), 7.552 (1.49), 7.975 (9.76), 8.874 (1.45), 9.053 (0.91), | |
| | 12.228 (1.51), 12.263 (2.11). | |
| 370 | 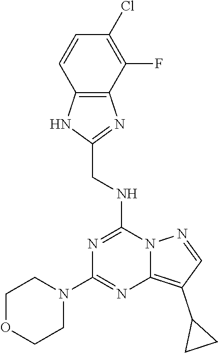 [SMILES: Fc1c(Cl)ccc2[nH]c(CNc3nc(N4CCOCC4)nc4c(C5CC5)cnn34)nc12] | 27% LC-MS (Method 1): Rₜ = 1.18 min; MS (ESIpos): m/z = 443 [M + H]⁺ |
| | N-[(5-chloro-4-fluoro-1H-benzimidazol-2-yl)methyl]-8- | |
| | cyclopropyl-2-(morpholin-4-yl)pyrazolo[1,5-a][1,3,5]triazin-4-amine | |
| | ¹H-NMR (400 MHz, DMSO-d6) δ [ppm]: 0.705 (2.03), 0.708 (1.27), | |
| | 0.717 (6.72), 0.722 (8.39), 0.730 (9.90), 0.735 (9.07), 0.743 (4.01), | |
| | 0.753 (1.54), 0.761 (2.02), 0.765 (4.48), 0.772 (7.75), 0.778 (6.02), | |
| | 0.786 (5.63), 0.790 (3.42), 0.794 (8.36), 0.799 (5.42), 0.806 (1.60), | |
| | 0.811 (2.58), 1.704 (1.34), 1.717 (2.54), 1.725 (2.62), 1.738 (4.04), | |
| | 1.746 (1.52), 1.751 (2.45), 1.758 (2.33), 1.772 (1.13), 2.153 (0.65), | |
| | 2.332 (2.02), 2.336 (0.89), 2.518 (2.07), 2.523 (8.12), 2.539 (1.24), | |
| | 2.678 (0.87), 3.292 (0.62), 3.299 (1.22), 3.307 (1.62), 3.390 (0.94), | |
| | 3.397 (0.71), 3.478 (9.03), 3.544 (9.38), 3.554 (10.49), 4.845 (8.92), | |
| | 4.860 (8.87), 7.237 (1.09), 7.259 (6.88), 7.269 (10.24), 7.713 (16.00), | |
| | 8.147 (0.62), 8.920 (1.39), 8.934 (2.51), 8.948 (1.22), 12.754 (2.28). | |
| 371 | 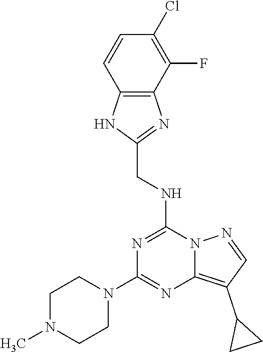 [SMILES: CN1CCN(c2nc(NCc3nc4c(F)c(Cl)ccc4[nH]3)n3ncc(C4CC4)c3n2)CC1] | 28% LC-MS (Method 1): Rₜ = 0.88 min; MS (ESIneg): m/z = 454 [M − H]⁻ |
| | N-[(5-chloro-4-fluoro-1H-benzimidazol-2-yl)methyl]-8- | |
| | cyclopropyl-2-(4-methylpiperazin-1-yl)pyrazolo[1,5- | |
| | a][1,3,5]triazin-4-amine | |
| | ¹H-NMR (400 MHz, DMSO-d6) δ [ppm]: 0.698 (1.16), 0.702 (0.77), | |
| | 0.710 (3.42), 0.715 (4.36), 0.722 (5.26), 0.728 (4.44), 0.736 (2.29), | |
| | 0.748 (0.72), 0.755 (0.86), 0.762 (2.23), 0.769 (4.47), 0.775 (2.93), | |
| | 0.783 (2.97), 0.787 (1.83), 0.791 (4.26), 0.796 (2.88), 0.802 (0.94), | |
| | 0.807 (1.27), 1.698 (0.69), 1.711 (1.32), 1.718 (1.36), 1.725 (0.66), | |
| | 1.731 (2.15), 1.740 (0.81), 1.744 (1.27), 1.753 (1.21), 1.765 (0.59), | |
| | 2.106 (16.00), 2.147 (3.23), 2.318 (0.52), 2.461 (0.44), 2.518 (6.49), | |
| | 2.523 (4.29), 2.539 (0.87), 2.660 (0.48), 3.556 (4.43), 4.832 (4.46), | |
| | 4.846 (4.48), 7.234 (0.62), 7.256 (3.33), 7.266 (2.77), 7.694 (11.05), | |
| | 8.152 (7.08), 8.888 (0.99), 12.753 (0.56). | |
| 372 | 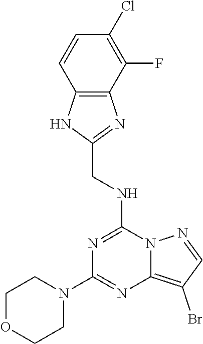 [SMILES: Fc1c(Cl)ccc2[nH]c(CNc3nc(N4CCOCC4)nc4c(Br)cnn34)nc12] | 27% LC-MS (Method 1): Rₜ = 1.15 min; MS (ESIpos): m/z = 481 [M + H]⁺ |
| | 8-bromo-N-[(5-chloro-4-fluoro-1H-benzimidazol-2-yl)methyl]-2- | |
| | (morpholin-4-yl)pyrazolo[1,5-a][1,3,5]triazin-4-amine | |
| | ¹H-NMR (400 MHz, DMSO-d6) δ [ppm]: 2.336 (0.56), 2.518 (8.62), | |
| | 2.522 (5.54), 2.539 (1.10), 2.673 (1.28), 2.678 (0.58), 3.487 (3.60), | |
| | 3.607 (5.12), 4.877 (4.77), 4.890 (4.82), 7.245 (0.78), 7.266 (3.96), | |
| | 7.278 (3.26), 8.043 (16.00), 9.200 (1.19), 12.779 (0.73). | |
| 373 | 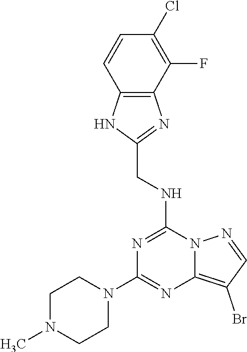 [SMILES: CN1CCN(c2nc(NCc3nc4c(F)c(Cl)ccc4[nH]3)n3ncc(Br)c3n2)CC1] | 28% LC-MS (Method 1): Rₜ = 0.84 min; MS (ESIpos): m/z = 494 [M + H]⁺ |
| | 8-bromo-N-[(5-chloro-4-fluoro-1H-benzimidazol-2-yl)methyl]-2- | |
| | (4-methylpiperazin-1-yl)pyrazolo[1,5-a][1,3,5]triazin-4-amine | |
| | ¹H-NMR (400 MHz, DMSO-d6) δ [ppm]: 2.075 (0.49), 2.114 (16.00), | |
| | 2.159 (1.38), 2.332 (1.17), 2.336 (0.55), 2.518 (5.97), 2.523 (4.22), | |
| | 2.539 (1.25), 2.678 (0.47), 3.406 (1.34), 3.603 (2.64), 4.863 (4.28), | |
| | 4.877 (4.29), 7.242 (0.56), 7.263 (3.32), 7.273 (2.69), 8.026 (13.23), | |
| | 8.145 (7.23), 9.160 (1.03), 12.779 (0.64). | |
| 374 | 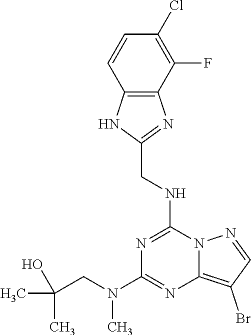 [SMILES: CN(CC(C)(C)O)c1nc(NCc2nc3c(F)c(Cl)ccc3[nH]2)n2ncc(Br)c2n1] | 13% LC-MS (Method 1): Rₜ = 1.15 min; MS (ESIpos): m/z = 497 [M + H]⁺ |
| | 1-[(8-bromo-4-{[(5-chloro-4-fluoro-1H-benzimidazol-2- | |
| | yl)methyl]amino}pyrazolo[1,5-a][1,3,5]triazin-2- | |
| | yl)(methyl)amino]-2-methylpropan-2-ol | |
| | ¹H-NMR (400 MHz, DMSO-d6) δ [ppm]: 0.700 (16.00), 1.082 (4.12), | |
| | 1.164 (0.49), 1.907 (0.43), 2.153 (0.52), 2.318 (0.96), 2.518 (12.88), | |
| | 2.523 (8.68), 2.539 (3.78), 2.660 (0.98), 3.017 (2.17), 3.114 (14.07), | |
| | 3.383 (5.44), 3.552 (1.30), 4.325 (2.81), 4.807 (3.88), 4.821 (3.99), | |
| | 4.881 (0.93), 4.905 (0.98), 7.219 (0.54), 7.241 (5.44), 7.225 (3.11), | |
| | 7.275 (1.85), 8.011 (14.47), 9.079 (1.39), 12.751 (1.50). | |
| 375 | 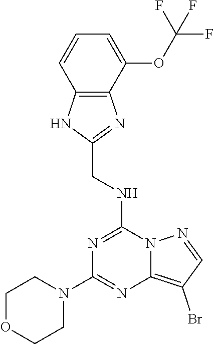 [SMILES: FC(F)(F)Oc1cccc2[nH]c(CNc3nc(N4CCOCC4)nc4c(Br)cnn34)nc12] | 32% LC-MS (Method 1): Rₜ = 1.17 min; MS (ESIpos): m/z = 513 [M + H]⁺ |
| | 8-bromo-2-(morpholin-4-yl)-N-{[4-(trifluoromethoxy)-1H- | |
| | benzimidazol-2-yl]methyl}pyrazolo[1,5-a][1,3,5]triazin-4-amine | |
| | ¹H-NMR (400 MHz, DMSO-d6) δ [ppm]: 2.332 (1.81), 2.336 (0.80), | |
| | 2.518 (11.47), 2.523 (7.39), 2.539 (0.99), 2.673 (1.85), 2.678 (0.81), | |
| | 3.456 (3.28), 3.586 (5.17), 4.891 (4.63), 4.904 (4.00), 7.118 (1.42), | |
| | 7.137 (2.24), 7.196 (4.40), 7.216 (5.23), 7.236 (2.25), 7.430 (2.71), | |
| | 7.449 (2.28), 7.997 (0.56), 8.042 (16.00), 9.207 (1.59), 12.675 (2.00), | |
| | 12.983 (0.40). | |
| 376 | 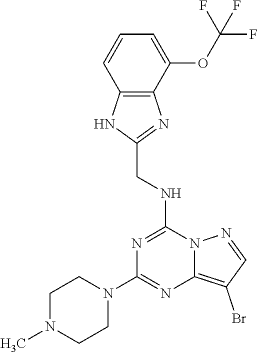 [SMILES: CN1CCN(c2nc(NCc3nc4c(OC(F)(F)F)cccc4[nH]3)n3ncc(Br)c3n2)CC1] | 23% LC-MS (Method 1): Rₜ = 0.86 min; MS (ESIpos): m/z = 526 [M + H]⁺ |
| | 8-bromo-2-(4-methylpiperazin-1-yl)-N-{[4-(trifluoromethoxy)-1H- | |
| | benzimidazol-2-yl]methyl}pyrazolo[1,5-a][1,3,5]triazin-4-amine | |
| | ¹H-NMR (400 MHz, DMSO-d6) δ [ppm]: 1.907 (0.45), 2.075 (1.66), | |
| | 2.097 (16.00), 2.216 (0.94), 2.332 (2.10), 2.336 (1.00), 2.518 (13.13), | |
| | 2.523 (8.50), 2.539 (1.08), 2.673 (2.00), 2.678 (0.89), 3.585 (2.58), | |
| | 3.602 (2.50), 4.877 (3.52), 4.891 (3.38), 7.116 (0.85), 7.135 (1.39), | |
| | 7.192 (3.16), 7.212 (4.48), 7.231 (1.99), 7.425 (1.36), 7.445 (1.22), | |
| | 8.024 (13.22), 8.150 (6.85), 9.160 (1.14), 12.673 (1.41). | |
| 377 | 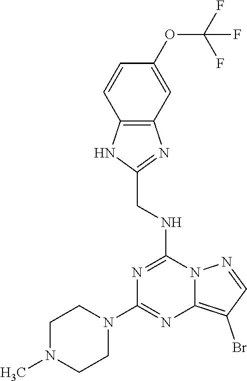 [SMILES: CN1CCN(c2nc(NCc3nc4cc(OC(F)(F)F)ccc4[nH]3)n3ncc(Br)c3n2)CC1] | 26% LC-MS (Method 1): Rₜ = 0.89 min; m/z = 526 [M + H]⁺ |
| | 8-bromo-2-(4-methylpiperazin-1-yl)-N-{[5-(trifluoromethoxy)-1H- | |
| | benzimidazol-2-yl]methyl}pyrazolo[1,5-a][1,3,5]triazin-4-amine | |
| | ¹H-NMR (400 MHz, DMSO-d6) δ [ppm]: 2.084 (16.00), 2.219 (0.84), | |
| | 2.522 (10.49), 2.539 (5.17), 3.592 (2.47), 3.602 (2.61), 4.850 (3.39), | |
| | 4.864 (3.51), 7.124 (1.03), 7.145 (1.12), 7.493 (0.65), 7.535 (0.70), | |
| | 8.023 (10.69), 8.147 (1.52), 9.135 (0.85), 9.149 (1.65), 9.165 (0.90), | |
| | 12.564 (0.48). | |
| 378 | 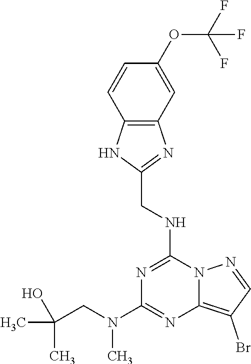 [SMILES: CN(CC(C)(C)O)c1nc(NCc2nc3cc(OC(F)(F)F)ccc3[nH]2)n2ncc(Br)c2n1] | 15% LC-MS (Method 1): Rₜ = 1.17 min; MS (ESIpos): m/z = 529 [M + H]⁺ |
| | 1-{[8-bromo-4-({[5-(trifluoromethoxy)-1H-benzimidazol-2- | |
| | yl]methyl}amino)pyrazolo[1,5-a][1,3,5]triazin-2- | |
| | yl](methyl)amino}-2-methylpropan-2-ol | |
| | ¹H-NMR (400 MHz, DMSO-d6) δ [ppm]: 0.674 (7.34), 0.686 (5.82), | |
| | 1.085 (2.48), 2.318 (0.51), 2.518 (6.84), 2.523 (4.57), 2.539 (0.95), | |
| | 2.678 (0.50), 3.036 (1.01), 3.114 (7.24), 3.387 (2.08), 3.406 (1.58), | |
| | 3.556 (0.73), 4.326 (1.31), 4.353 (0.93), 4.803 (1.99), 4.816 (2.03), | |
| | 4.888 (0.61), 4.905 (0.54), 7.102 (0.52), 7.124 (1.16), 7.145 (0.76), | |
| | 7.354 (0.69), 7.444 (0.77), 7.465 (0.75), 7.478 (0.46), 7.496 (1.08), | |
| | 7.585 (0.54), 7.606 (0.51), 8.009 (16.00), 9.058 (1.05), 12.515 (0.82), | |
| | 12.550 (0.85). | |
| 379 | 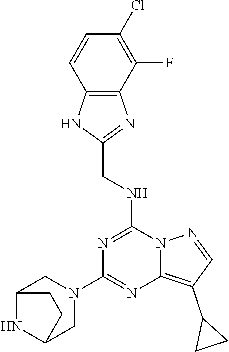 [SMILES: Fc1c(Cl)ccc2[nH]c(CNc3nc(N4CC5CCC(C4)N5)nc4c(C5CC5)cnn34)nc12] | 8% LC-MS (Method 2): Rₜ = 1.09 min; MS (ESIpos): m/z = 468 [M + H]⁺ |
| | N-[(5-chloro-4-fluoro-1H-benzimidazol-2-yl)methyl]-8- | |
| | cyclopropyl-2-(3,8-diazabicyclo[3.2.1]octan-3-yl)pyrazolo[1,5- | |
| | a][1,3,5]triazin-4-amine | |
| | ¹H-NMR (400 MHz, DMSO-d6) δ [ppm]: 0.716 (10.52), 0.724 (10.88), | |
| | 0.728 (10.98), 0.771 (9.12), 0.783 (6.85), 0.791 (9.30), 1.100 (1.94), | |
| | 1.168 (1.50), 1.229 (4.25), 1.582 (2.30), 1.690 (2.41), 1.703 (3.48), | |
| | 1.710 (3.97), 1.723 (5.36), 1.736 (4.12), 1.747 (10.38), 1.902 (0.85), | |
| | 1.983 (1.32), 2.288 (1.12), 3.007 (3.72), 3.070 (1.13), 3.573 (1.71), | |
| | 3.587 (2.83), 3.678 (2.41), 3.699 (2.58), 3.845 (1.24), 4.014 (0.44), | |
| | 4.241 (2.70), 4.639 (0.75), 4.824 (10.27), 5.756 (1.17), 7.256 (8.83), | |
| | 7.673 (0.78), 7.721 (16.00), 7.872 (0.60), 8.048 (0.88), 8.156 (1.08), | |
| | 9.010 (3.03). | |
| 380 | 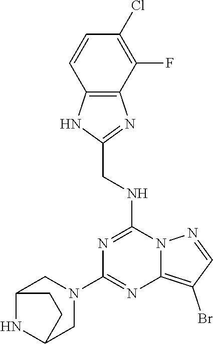 [SMILES: Fc1c(Cl)ccc2[nH]c(CNc3nc(N4CC5CCC(C4)N5)nc4c(Br)cnn34)nc12] | 12% LC-MS (Method 2): Rₜ = 1.03 min; MS (ESIpos): m/z = 506 [M + H]⁺ |
| | 8-bromo-N-[(5-chloro-4-fluoro-1H-benzimidazol-2-yl)methyl]-2- | |
| | (3,8-diazabicyclo[3.2.1]octan-3-yl)pyrazolo[1,5-a][1,3,5]triazin-4- | |
| | amine | |
| | ¹H-NMR (400 MHz, DMSO-d6) δ [ppm]: 1.035 (1.24), 1.052 (2.57), | |
| | 1.070 (1.33), 1.104 (0.49), 1.176 (1.14), 1.232 (0.94), 1.523 (1.05), | |
| | 1.690 (0.65), 1.751 (6.40), 1.907 (0.60), 2.518 (12.88), 2.523 (8.55), | |
| | 2.540 (3.82), 3.074 (0.98), 3.385 (2.97), 3.412 (0.92), 3.425 (0.89), | |
| | 3.575 (0.80), 3.590 (2.46), 3.656 (0.75), 3.667 (0.99), 3.681 (0.93), | |
| | 3.701 (0.88), 3.710 (0.66), 3.715 (0.75), 3.828 (0.64), 3.863 (1.63), | |
| | 4.209 (0.58), 4.350 (0.70), 4.644 (0.82), 4.858 (5.16), 5.759 (2.72), | |
| | 7.266 (4.44), 8.062 (16.00), 8.155 (1.51), 9.297 (0.62). | |
| 381 | 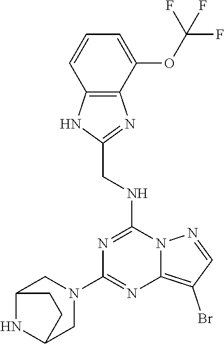 [SMILES: FC(F)(F)Oc1cccc2[nH]c(CNc3nc(N4CC5CCC(C4)N5)nc4c(Br)cnn34)nc12] | 12% LC-MS (Method 2): Rₜ = 1.10 min; MS (ESIpos): m/z = 538 [M + H]⁺ |
| | 8-bromo-2-[(1R,5S)-3,8-diazabicyclo[3.2.1]octan-3-yl]-N-{[4- | |
| | (trifluoromethoxy)-1H-benzimidazol-2-yl]methyl}pyrazolo[1,5- | |
| | a][1,3,5]triazin-4-amine | |
| | ¹H-NMR (400 MHz, DMSO-d6) δ [ppm]: 0.883 (0.42), 0.852 (0.67), | |
| | 1.035 (0.45), 1.104 (16.00), 1.232 (2.80), 1.349 (0.67), 1.424 (0.55), | |
| | 1.493 (0.54), 2.332 (1.59), 2.336 (0.72), 2.518 (9.65), 2.523 (6.72), | |
| | 2.673 (1.62), 2.679 (0.75), 2.765 (0.59), 2.863 (0.59), 3.074 (5.17), | |
| | 3.167 (0.65), 3.424 (0.76), 3.986 (0.55), 4.205 (0.57), 4.845 (4.73), | |
| | 7.109 (0.88), 7.129 (1.35), 7.185 (2.94), 7.204 (4.26), 7.225 (1.91), | |
| | 7.417 (1.21), 7.437 (1.04), 8.010 (12.18), 9.147 (0.48), 12.659 (0.51). | |
| 382 | 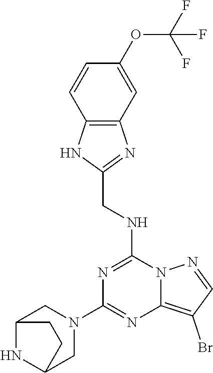 [SMILES: FC(F)(F)Oc1ccc2[nH]c(CNc3nc(N4CC5CCC(C4)N5)nc4c(Br)cnn34)nc2c1] | 45% LC-MS (Method 2): Rₜ = 1.12 min; MS (ESIpos): m/z = 538 [M + H]⁺ |
| | 8-bromo-2-[(1R,5S)-3,8-diazabicyclo[3.2.1]octan-3-yl]-N-{[5- | |
| | (trifluoromethoxy)-1H-benzimidazol-2-yl]methyl}pyrazolo[1,5- | |
| | a][1,3,5]triazin-4-amine | |
| | ¹H-NMR (400 MHz, DMSO-d6) δ [ppm]: 0.946 (0.20), 1.104 (16.00), | |
| | 1.232 (0.36), 1.258 (0.25), 1.347 (0.21), 1.424 (0.32), 1.465 (0.18), | |
| | 2.332 (0.53), 2.518 (3.75), 2.523 (2.42), 2.673 (0.54), 2.773 (0.23), | |
| | 2.841 (0.22), 3.074 (5.18), 3.144 (0.23), 3.974 (0.17), 4.190 (0.19), | |
| | 4.820 (1.35), 7.121 (0.34), 7.473 (0.19), 7.494 (0.20), 7.530 (0.28), | |
| | 8.003 (4.00), 9.118 (0.17), 12.547 (0.18). | |
| 383 | 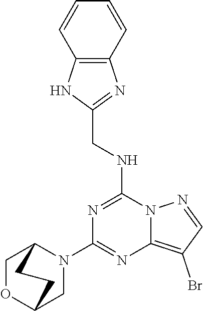 [SMILES: Brc1cnn2c(NCc3nc4ccccc4[nH]3)nc(N3C[C@H]4CC[C@@H]3CO4)nc12] | 23% LC-MS (Method 2): Rₜ = 1.04 min; MS (ESIpos): m/z = 455 [M + H]⁺ |
| | N-[(1H-benzimidazol-2-yl)methyl]-8-bromo-2-{[1R or S- | |
| | (1R*,4R*)]-2-oxa-5-azabicyclo[2.2.2]octan-5-yl}pyrazolo[1,5- | |
| | a][1,3,5]triazin-4-amine (Enantiomer 1) | |
| | ¹H-NMR (400 MHz, DMSO-d6) δ [ppm]: 0.851 (0.84), 1.173 (1.33), | |
| | 1.232 (2.98), 1.548 (2.30), 1.575 (2.91), 1.614 (1.66), 1.899 (2.28), | |
| | 2.074 (0.87), 2.083 (1.98), 2.518 (16.00), 2.522 (10.94), 3.471 (0.99), | |
| | 3.500 (1.44), 3.558 (1.54), 3.589 (2.42), 3.634 (1.11), 3.696 (5.46), | |
| | 3.898 (1.63), 3.925 (5.33), 4.427 (2.51), 4.628 (1.54), 4.816 (3.65), | |
| | 4.827 (3.81), 4.901 (2.27), 7.122 (4.26), 7.127 (4.32), 7.134 (3.98), | |
| | 7.377 (1.78), 7.394 (1.84), 7.422 (1.07), 7.515 (1.87), 7.532 (2.67), | |
| | 8.012 (14.58), 9.083 (2.27), 12.231 (2.44), 12.255 (1.59). | |

### Example 384

### N-[(4-fluoro-1H-benzimidazol-2-yl)methyl]-2-(8-methyl-3,8-diazabicyclo[3.2.1]octan-3-yl)-8-(trifluoromethyl)pyrazolo[1,5-a][1,3,5]triazin-4-amine

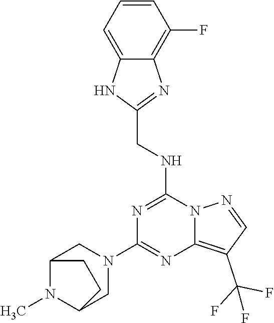 [SMILES: CN1C2CCC1CN(c1nc(NCc3nc4c(F)cccc4[nH]3)n3ncc(C(F)(F)F)c3n1)C2]

N-[(4-fluoro-1-{[2-(trimethylsilyl)ethoxy]methyl}-1H-benzimidazol-2-yl)methyl]-2-(8-methyl-3,8-diazabicyclo[3.2.1]octan-3-yl)-8-(trifluoromethyl)pyrazolo[1,5-a][1,3,5]triazin-4-amine (Intermediate 201, 188 mg, 310 μmol) was provided in dichloromethane (5.0 mL). Trifluoroacetic acid (2.5 mL, 32.4 mmol; CAS-RN:[76-05-1]) was added and the mixture was stirred for 18 h at room temperature. The reaction mixture was concentrated under vacuum, the residue was purified by flash chromatography (silica gel, dichloromethane/ethanol gradient) to give 88.0 mg (54% yield) of the title compound.

LC-MS (Method 2): Rₜ=1.16 min; MS (ESIpos): m/z=476 [M+H]⁺

¹H-NMR (400 MHz, DMSO-d6) δ[ppm]: 0.947 (0.62), 1.035 (0.45), 1.052 (0.91), 1.070 (0.45), 1.232 (1.38), 1.542 (0.59), 1.768 (0.60), 2.100 (16.00), 2.522 (5.16), 2.827 (1.09), 2.855 (1.80), 2.921 (1.01), 2.953 (0.92), 3.078 (1.03), 3.987 (0.74), 4.017 (0.70), 4.170 (0.92), 4.202 (0.89), 4.846 (3.34), 5.759 (0.43), 6.915 (0.51), 6.937 (0.80), 6.963 (0.72), 7.094 (0.74), 7.106 (0.89), 7.113 (1.54), 7.125 (1.59), 7.134 (0.96), 7.146 (0.87), 7.226 (1.22), 7.247 (0.95), 8.229 (5.77), 9.281 (0.83), 12.559 (0.94).

### Example 385

### N-{[5-(3-fluorophenyl)-4H-1,2,4-triazol-3-yl]methyl}-2-(morpholin-4-yl)-8-(trifluoromethyl)pyrazolo[1,5-a][1,3,5]triazin-4-amine

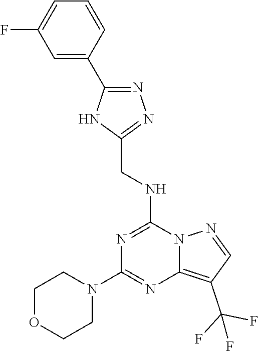 [SMILES: Fc1cccc(-c2nnc(CNc3nc(N4CCOCC4)nc4c(C(F)(F)F)cnn34)[nH]2)c1]

2-{[2-(morpholin-4-yl)-8-(trifluoromethyl)pyrazolo[1,5-a][1,3,5]triazin-4-yl]amino}acetohydrazide (Intermediate 204, 117 mg, 260 μmol) and 3-fluorobenzene-1-carboximidamide hydrogen chloride (54.4 mg, 312 μmol, CAS-RN:[75207-72-6]) were dissolved in DMF (3.0 mL). Sodiumethylate (35.4 mg, 520 μmol; CAS-RN:[141-52-6]) was added and the reaction mixture was stirred for 45 min at 180° C. in a microwave. The reaction mixture was poured into water and was extracted with ethyl acetate. The combined organic layers were concentrated under reduced pressure. The residue was purified HPLC (HT acidic) to give 63.5 mg (53% yield) of the title compound.

LC-MS (Method 1): Rₜ=1.17 min; MS (ESIpos): m/z=464 [M+H]⁺

¹H-NMR (400 MHz, DMSO-d6) δ[ppm]: 2.323 (1.11), 2.327 (1.56), 2.332 (1.11), 2.518 (5.29), 2.523 (3.52), 2.660 (0.48), 2.665 (1.11), 2.669 (1.56), 2.673 (1.08), 3.549 (6.00), 3.661 (8.73), 3.672 (10.50), 4.800 (7.25), 7.243 (1.19), 7.249 (1.32), 7.264 (2.51), 7.270 (2.67), 7.285 (1.45), 7.291 (1.51), 7.494 (1.67), 7.509 (2.20), 7.514 (3.20), 7.529 (3.15), 7.534 (2.12), 7.549 (1.64), 7.677 (2.43), 7.681 (2.91), 7.684 (2.99), 7.687 (2.70), 7.703 (2.54), 7.706 (2.99), 7.709 (2.86), 7.713 (2.62), 7.803 (3.99), 7.806 (5.66), 7.809 (3.94), 7.822 (3.70), 7.825 (4.95), 7.828 (3.41), 8.247 (16.00), 9.302 (1.64), 14.112 (0.50).

### Example 386

### N-{[5-(3,5-difluorophenyl)-4H-1,2,4-triazol-3-yl]methyl}-2-(morpholin-4-yl)-8-(trifluoromethyl)pyrazolo[1,5-a][1,3,5]triazin-4-amine

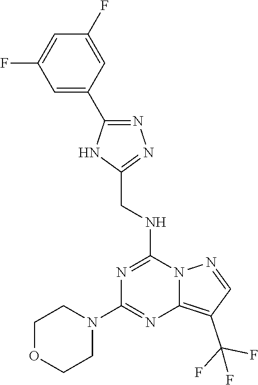 [SMILES: Fc1cc(F)cc(-c2nnc(CNc3nc(N4CCOCC4)nc4c(C(F)(F)F)cnn34)[nH]2)c1]

2-{[2-(morpholin-4-yl)-8-(trifluoromethyl)pyrazolo[1,5-a][1,3,5]triazin-4-yl]amino}acetohydrazide (Intermediate 204, 117 mg, 260 μmol) and 3,5-difluorobenzene-1-carboximidamide hydrogen chloride (60.0 mg, 312 μmol) were dissolved in DMF (3.0 mL). Sodiumethylate (35.4 mg, 520 μmol; CAS-RN:[141-52-6]) was added and the reaction mixture was stirred for 45 min at 180° C. in a microwave. The reaction mixture was poured into water and was extracted with ethyl acetate. The combined organic layers were concentrated under reduced pressure. The residue was purified HPLC (HT acidic) to give 84.2 mg (67% yield) of the title compound.

LC-MS (Method 1): Rₜ=1.23 min; MS (ESIpos): m/z=482 [M+H]⁺

¹H-NMR (400 MHz, DMSO-d6) δ[ppm]: 2.074 (1.21), 2.323 (0.78), 2.327 (1.13), 2.332 (0.82), 2.518 (4.06), 2.523 (2.62), 2.539 (0.41), 2.665 (0.78), 2.669 (1.11), 2.673 (0.80), 3.552 (5.62), 3.665 (8.96), 4.812 (11.08), 7.295 (0.93), 7.300 (1.94), 7.306 (1.28), 7.318 (1.91), 7.324 (3.81), 7.329 (2.45), 7.341 (1.05), 7.347 (1.89), 7.353 (1.22), 7.561 (0.78), 7.573 (5.15), 7.578 (6.82), 7.582 (4.28), 7.594 (6.49), 7.600 (5.35), 7.612 (0.91), 8.250 (16.00), 9.318 (1.07).

### Example 387

### 1-[(4-{[(5-chloro-4-fluoro-1H-benzimidazol-2-yl)methyl]amino}-8-cyclopropylpyrazolo[1,5-a][1,3,5]triazin-2-yl)(methyl)amino]-2-methylpropan-2-ol

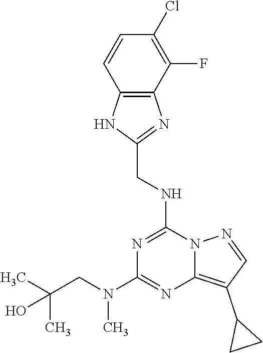 [SMILES: CN(CC(C)(C)O)c1nc(NCc2nc3c(F)c(Cl)ccc3[nH]2)n2ncc(C3CC3)c2n1]

The crude product of N-[(5-chloro-4-fluoro-1H-benzimidazol-2-yl)methyl]-8-cyclopropyl-2-(methanesulfonyl)pyrazolo[1,5-a][1,3,5]triazin-4-amine (Intermediate 206, 100 mg) and 2-methyl-1-(methylamino)propan-2-ol (80 μL, 690 μmol) were provided in acetonitrile (3.0 mL), N,N-diisopropylethylamine (120 μL, μmol; CAS-RN:[7087-68-5]) was added and the mixture was stirred at 70° C. over night. The mixture was concentrated under reduced pressure. The residue was purified by HPLC twice (1. HT acidic; 2. Instrument: Waters Autopurificationsystem; Column: XBrigde C18 5μ, 100×30 mm; eluent A: water+0.1 vol % formic acid; eluent B: methanol; gradient: 0.0-0.5 min 27% B (35-70 mL/min), 0.5-5.5 min 55-80% B; flow: 70 mL/min; temperature: 25° C.; DAD scan: 210-400 nm) to give 4.3 mg of the title compound.

LC-MS (Method 1): Rₜ=1.13 min; MS (ESIpos): m/z=459 [M+H]⁺

¹H-NMR (400 MHz, DMSO-d6) δ[ppm]: 0.703 (7.07), 0.725 (3.03), 0.739 (6.19), 0.745 (9.30), 0.750 (6.31), 0.752 (6.54), 0.758 (9.03), 0.764 (8.16), 0.787 (4.71), 0.851 (0.51), 1.076 (1.77), 1.137 (0.85), 1.169 (0.88), 1.232 (1.72), 1.737 (0.99), 2.332 (2.80), 2.336 (1.24), 2.518 (16.00), 2.523 (10.93), 2.539 (4.41), 2.673 (2.85), 2.678 (1.22), 3.009 (0.96), 3.083 (4.55), 3.358 (3.14), 3.506 (0.58), 4.272 (0.71), 4.791 (2.12), 4.872 (0.60), 7.245 (4.00), 7.682 (3.40), 8.796 (0.84), 12.740 (0.50).

### Example 388

### 2-[(2S)-2,4-dimethylpiperazin-1-yl]-N-[(4-fluoro-1H-benzimidazol-2-yl)methyl]-8-(trifluoromethyl)pyrazolo[1,5-a][1,3,5]triazin-4-amine

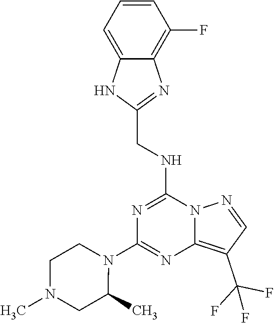 [SMILES: C[C@H]1CN(C)CCN1c1nc(NCc2nc3c(F)cccc3[nH]2)n2ncc(C(F)(F)F)c2n1]

A solution of 2-[(2S)-2,4-dimethylpiperazin-1-yl]-N-[(4-fluoro-1-{[2-(trimethylsilyl)ethoxy]methyl}-1H-benzimidazol-2-yl)methyl]-8-(trifluoromethyl)pyrazolo[1,5-a][1,3,5]triazin-4-amine (Intermediate 214, 38.0 mg) in dichloromethane (2.0 mL) and trifluoroacetic acid (1.5 mL, 19 mmol; CAS-RN:[76-05-1]) was stirred at 50° C. for 22 h. The reaction mixture was concentrated. The residue was purified by flash chromatography (silica amino-phase; gradient dichloromethane/ethanol) to give 12.0 mg of the title compound.

LC-MS (Method 2): Rₜ=1.22 min; MS (ESIpos): m/z=464 [M+H]⁺

¹H-NMR (400 MHz, DMSO-d6) δ[ppm]: 0.798 (1.83), 0.803 (1.11), 0.815 (2.04), 0.822 (2.11), 0.835 (1.71), 0.840 (1.44), 0.851 (1.64), 0.867 (0.95), 0.884 (0.88), 0.886 (1.18), 0.902 (1.20), 0.904 (2.04), 0.920 (0.78), 0.922 (1.22), 0.991 (0.54), 1.009 (0.56), 1.016 (1.12), 1.035 (1.33), 1.052 (0.87), 1.071 (0.73), 1.078 (0.65), 1.089 (0.48), 1.109 (0.42), 1.124 (0.42), 1.142 (0.44), 1.159 (0.50), 1.203 (0.76), 1.232 (5.41), 1.256 (1.26), 1.549 (0.49), 1.703 (0.47), 1.875 (0.78), 1.904 (0.82), 1.987 (0.44), 2.066 (1.03), 2.103 (16.00), 2.174 (0.67), 2.326 (3.26), 2.332 (2.31), 2.336 (1.05), 2.413 (0.47), 2.438 (0.49), 2.518 (12.17), 2.522 (8.32), 2.668 (3.40), 2.673 (2.55), 2.678 (1.24), 2.944 (0.53), 2.968 (0.97), 3.000 (0.57), 4.258 (0.60), 4.278 (0.91), 4.310 (0.86), 4.870 (1.99), 6.911 (0.68), 6.931 (0.97), 6.958 (0.89), 7.091 (0.73), 7.103 (0.93), 7.110 (1.49), 7.122 (1.58), 7.130 (0.94), 7.143 (0.82), 7.222 (1.68), 7.242 (1.24), 8.242 (6.25), 9.286 (0.73), 12.571 (1.19).

### Example 389

### N-[(1H-benzimidazol-2-yl)methyl]-8-bromo-2-[(1-methylpiperidin-4-yl)oxy]pyrazolo[1,5-a][1,3,5]triazin-4-amine

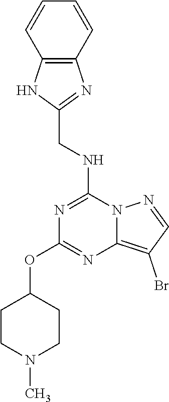 [SMILES: CN1CCC(Oc2nc(NCc3nc4ccccc4[nH]3)n3ncc(Br)c3n2)CC1]

To 1-methylpiperidin-4-ol (98 μL, 830 μmol) in tetrahydrofuran (3.0 mL) at 0° C. sodium hydride (20.5 mg, 60% in mineral oil, 512 μmol; CAS-RN:[7646-69-7]) was added and the mixture was stirred for 25 min. N-[(1H-benzimidazol-2-yl)methyl]-8-bromo-2-(methanesulfonyl)pyrazolo[1,5-a][1,3,5]triazin-4-amine (Intermediate 9, 100 mg, 213 μmol) was added and the mixture was stirred for 20 h at room temperature. 1-methylpiperidin-4-ol (98 μL, 830 μmol) and sodium hydride (20.5 mg, 60% in mineral oil, 512 μmol; CAS-RN:[7646-69-7]) were added and the mixture was stirred 24 h at room temperature. Water was added and the mixture was extracted with ethyl acetate. The combined organic layers were concentrated. The residue was purified by HPLC (HT basic) to give 32.5 mg (33% yield) of the title compound.

LC-MS (Method 2): Rₜ=1.01 min; MS (ESIneg): m/z=455 [M−H]⁻

¹H-NMR (500 MHz, DMSO-d6) δ[ppm]: 1.551 (0.48), 1.569 (1.30), 1.587 (1.41), 1.605 (0.61), 1.612 (0.51), 1.752 (0.62), 1.831 (1.35), 1.850 (1.09), 1.897 (0.66), 2.018 (0.77), 2.036 (1.36), 2.056 (0.81), 2.121 (16.00), 2.514 (2.75), 2.518 (2.48), 2.522 (2.09), 4.831 (0.48), 4.840 (0.69), 4.848 (0.88), 4.856 (0.71), 4.865 (0.50), 4.896 (5.59), 7.132 (1.86), 7.141 (1.96), 7.393 (0.71), 7.532 (0.75), 8.224 (9.96), 9.518 (0.50), 12.307 (0.59).

In analogy to the procedures described above, the following examples were prepared using the appropriate intermediates as starting materials.

TABLE 3
Example 390-412

| | Structure | Yield |
|---|---|---|
| | IUPAC-Name | Analytics |
| Example | NMR | |
| 390 | 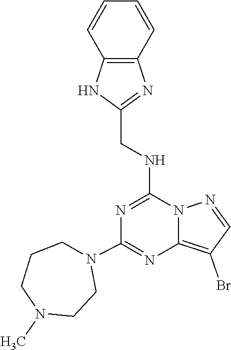 [SMILES: CN1CCCN(c2nc(NCc3nc4ccccc4[nH]3)n3ncc(Br)c3n2)CC1] | 49% LC-MS (Method 2): Rₜ = 1.04 min; MS (ESIpos): m/z = 456 [M + H]⁺ |
| | N-[(1H-benzimidazol-2-yl)methyl]-8-bromo-2-(4-methyl-1,4- | |
| | diazepan-1-yl)pyrazolo[1,5-a][1,3,5]triazin-4-amine | |
| | ¹H-NMR (500 MHz, DMSO-d6) [ppm]: 1.230 (0.63), 1.488 (1.35), | |
| | 1.752 (0.88), 1.804 (1.93), 1.891 (9.62), 1.902 (1.74), 1.986 (2.51), | |
| | 2.073 (1.11), 2.189 (6.15), 2.267 (3.83), 2.514 (4.11), 2.518 (3.36), | |
| | 2.522 (2.65), 2.539 (0.53), 2.571 (1.80), 3.539 (1.42), 3.551 (2.27), | |
| | 3.582 (2.76), 3.635 (2.08), 3.647 (3.57), 3.660 (1.97), 3.727 (1.82), | |
| | 4.802 (3.05), 4.810 (3.20), 4.846 (2.17), 7.114 (3.98), 7.307 (0.50), | |
| | 7.380 (2.03), 7.393 (2.01), 7.512 (2.12), 7.526 (1.88), 7.998 (16.00), | |
| | 9.036 (0.80), 9.072 (1.24), 12.254 (1.65). | |
| 391 | 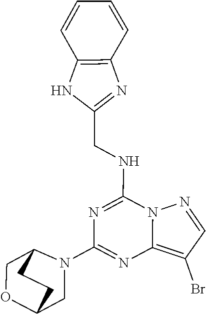 [SMILES: Brc1cnn2c(NCc3nc4ccccc4[nH]3)nc(N3C[C@H]4CC[C@@H]3CO4)nc12] | 26% LC-MS (Method 2): Rₜ = 1.04 min; MS (ESIpos): m/z = 455 [M + H]⁺ |
| | N-[(1H-benzimidazol-2-yl)methyl]-8-bromo-2-{[1R or S- | |
| | (1R*,4R*)]-2-oxa-5-azabicyclo[2.2.2]octan-5-yl}pyrazolo[1, 5- | |
| | a][1,3,5]triazin-4-amine (Enantiomer 2) | |
| | ¹H-NMR (400 MHz,DMSO-d6) 6 [ppm]: 0.851 (0.93),0.873 (0.76), | |
| | 1.137 (0.91), 1.233 (2.86), 1.549 (2.08), 1.575 (2.65), 1.614 (1.51), | |
| | 1.899 (2.09), 2.084 (5.30), 2.518 (16.00), 2.522 (10.77), 3.470 (0.93), | |
| | 3.503 (1.33), 3.561 (1.39), 3.590 (2.22), 3.634 (1.03), 3.697 (5.03), | |
| | 3.898 (1.53), 3.926 (4.92), 4.428 (2.32), 4.628 (1.47), 4.816 (3.35), | |
| | 4.827 (3.41), 4.902 (2.09), 7.122 (3.94), 7.127 (3.97), 7.377 (1.65), | |
| | 7.396 (1.69), 7.423 (0.95), 7.515 (1.69), 7.531 (2.43), 8.012 (13.52), | |
| | 9.085 (2.06), 12.233 (2.20), 12.257 (1.46). | |
| 392 | 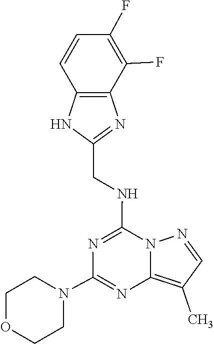 [SMILES: Cc1cnn2c(NCc3nc4c(F)c(F)ccc4[nH]3)nc(N3CCOCC3)nc12] | 29% yield LC-MS (Method 1): Rₜ = 1.02 min; MS (ESIpos): m/z = 401 [M + H]⁺ |
| | N-[(4,5-difluoro-1H-benzimidazol-2-yl)methyl]-8-methyl-2- | |
| | (morpholin-4-yl)pyrazolo[1,5-a][1,3,5]triazin-4-amine | |
| | ¹H-NMR (400 MHz, DMSO-d6) δ [ppm]: 2.029 (16.00), 2.332 (0.48), | |
| | 2.518 (2.19), 2.523 (1.44), 2.673 (0.48), 3.482 (3.83), 3.494 (3.33), | |
| | 3.562 (3.91), 3.574 (4.31), 3.585 (2.40), 4.849 (3.22), 4.863 (3.20), | |
| | 7.174 (0.67), 7.190 (0.89), 7.201 (1.47), 7.214 (1.75), 7.779 (4.91), | |
| | 8.159 (0.59), 8.927 (0.78), 12.679 (0.76). | |
| 393 | 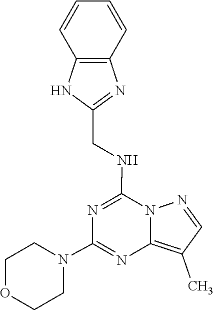 [SMILES: Cc1cnn2c(NCc3nc4ccccc4[nH]3)nc(N3CCOCC3)nc12] | 38% LC-MS (Method 1): Rₜ = 0.76 min; MS (ESIneg): m/z = 363 [M − H]⁻ |
| | N-[(1H-benzimidazol-2-yl)methyl]-8-methyl-2-(morpholin-4- | |
| | yl)pyrazolo[1,5-a][1,3,5]triazin-4-amine | |
| | ¹H-NMR (400 MHz, DMSO-d6) δ [ppm]: 2.000 (0.54), 2.032 (16.00), | |
| | 2.332 (0.45), 2.518 (2.16), 2.523 (1.41), 2.539 (0.43), 3.486 (3.89), | |
| | 3.497 (3.32), 3.575 (4.01), 3.587 (4.48), 3.598 (2.51), 4.849 (3.24), | |
| | 4.863 (3.22), 7.109 (1.14), 7.119 (1.65), 7.125 (1.47), 7.131 (1.81), | |
| | 7.142 (1.28), 7.396 (0.87), 7.413 (0.81), 7.520 (0.88), 7.537 (0.79), | |
| | 7.775 (5.86), 8.179 (0.60), 8.838 (0.68), 8.853 (1.41), 8.867 (0.68), | |
| | 12.252 (1.12). | |
| 394 | 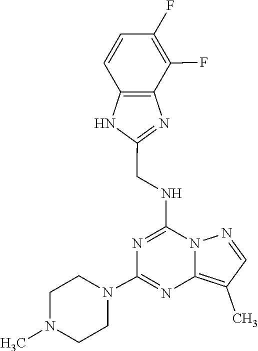 [SMILES: Cc1cnn2c(NCc3nc4c(F)c(F)ccc4[nH]3)nc(N3CCN(C)CC3)nc12] | 41% LC-MS (Method 1): Rₜ = 0.72 min; MS (ESIpos): m/z = 414 [M + H]⁺ |
| | N-[(4,5-difluoro-1H-benzimidazol-2-yl)methyl]-8-methyl-2-(4- | |
| | methylpiperazin-1-yl)pyrazolo[1,5-a][1,3,5]triazin-4-amine | |
| | ¹H-NMR (400 MHz, DMSO-d6) δ [ppm]: 2.021 (16.00), 2.114 (12.37), | |
| | 2.162 (2.95), 2.214 (0.51), 2.518 (3.44), 2.523 (2.22), 3.316 (3.96), | |
| | 3.578 (3.49), 4.836 (3.20), 4.850 (3.21), 7.169 (0.78), 7.186 (1.00), | |
| | 7.197 (1.38), 7.211 (1.39), 7.762 (5.46), 8.148 (4.32), 8.879 (0.71), | |
| | 12.678 (0.58). | |
| 395 | 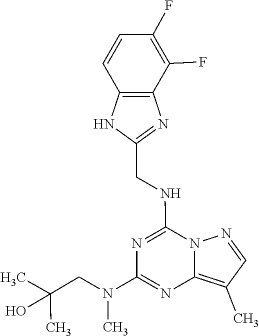 [SMILES: Cc1cnn2c(NCc3nc4c(F)c(F)ccc4[nH]3)nc(N(C)CC(C)(C)O)nc12] | 7% LC-MS (Method 1): Rₜ = 0.95 min; MS (ESIpos): m/z = 417 [M + H]⁺ |
| | 1-[(4-{[(4, 5-difluoro-1H-benzimidazol-2-yl)methyl]amino}-8- | |
| | methylpyrazolo[1,5-a][1,3,5]triazin-2-yl)(methyl)amino]-2- | |
| | methylpropan-2-ol | |
| | ¹H-NMR (400 MHz, DMSO-d6) δ [ppm]: 0.715 (10.73), 0.922 (1.31), | |
| | 1.026 (2.42), 1.075 (1.93), 1.146 (6.17), 1.153 (3.99), 1.173 (1.52), | |
| | 2.025 (14.81), 2.075 (0.50), 2.264 (1.53), 2.332 (2.38), 2.336 (1.08), | |
| | 2.518 (13.15), 2.523 (8.21), 2.539 (3.02), 2.545 (0.90), 2.981 (3.62), | |
| | 3.091 (8.08), 3.177 (2.05), 3.214 (0.49), 3.231 (1.99), 3.384 (4.30), | |
| | 3.433 (2.43), 3.535 (1.42), 3.606 (0.46), 4.205 (0.40), 4.283 (1.77), | |
| | 4.599 (1.01), 4.643 (0.56), 4.794 (3.75), 5.042 (0.46), 7.160 (2.90), | |
| | 7.173 (4.76), 7.185 (4.90), 7.359 (0.65), 7.376 (0.64), 7.466 (0.84), | |
| | 7.501 (0.65), 7.525 (0.51), 7.746 (16.00), 7.873 (0.49), 7.892 (1.03), | |
| | 8.143 (3.34), 8.286 (0.41), 8.798 (1.53), 12.654 (2.81), 13.113 (0.51). | |
| 396 | 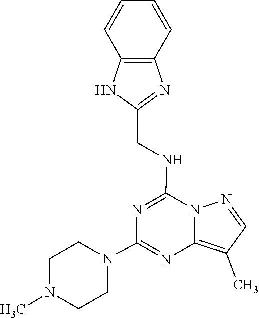 [SMILES: Cc1cnn2c(NCc3nc4ccccc4[nH]3)nc(N3CCN(C)CC3)nc12] | 26% LC-MS (Method 1): Rₜ = 0.54 min; MS (ESIneg): m/z = 376 [M − H]⁻ |
| | N-[(1H-benzimidazol-2-yl)methyl]-8-methyl-2-(4-methylpiperazin- | |
| | 1-yl)pyrazolo[1,5-a][1,3,5]triazin-4-amine | |
| | ¹H-NMR (400 MHz, DMSO-d6) δ [ppm]: 2.025 (16.00), 2.112 (13.26), | |
| | 2.175 (3.13), 2.331 (0.65), 2.518 (3.30), 2.523 (2.14), 3.597 (3.36), | |
| | 4.841 (2.97), 4.855 (2.97), 7.108 (1.13), 7.117 (1.69), 7.124 (1.44), | |
| | 7.130 (1.83), 7.139 (1.25), 7.393 (0.82), 7.410 (0.76), 7.519 (0.84), | |
| | 7.536 (0.73), 7.756 (5.92), 8.784 (0.65), 8.798 (1.31), 12.254 (1.51). | |
| 397 | 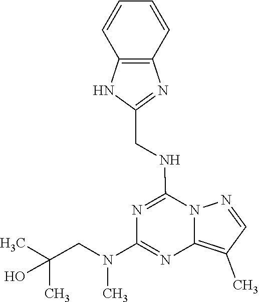 [SMILES: Cc1cnn2c(NCc3nc4ccccc4[nH]3)nc(N(C)CC(C)(C)O)nc12] | 13% LC-MS (Method 1): Rₜ = 0.75 min; MS (ESIpos): m/z = 381 [M + H]⁺ |
| | 1-[(4-{[(1H-benzimidazol-2-yl)methyl]amino}-8- | |
| | methylpyrazolo[1,5-a][1,3,5]triazin-2-yl)(methyl)amino]-2- | |
| | methylpropan-2-ol | |
| | ¹H-NMR (400 MHz, DMSO-d6) δ [ppm]: 0.794 (9.50), 1.083 (2.01), | |
| | 1.152 (0.60), 1.164 (0.80), 2.026 (12.48), 2.266 (0.58), 2.518 (13.84), | |
| | 2.523 (8.67), 2.539 (1.23), 3.102 (8.46), 3.235 (1.62), 3.436 (4.23), | |
| | 4.336 (1.24), 4.805 (3.17), 7.108 (5.52), 7.121 (6.01), 7.395 (1.97), | |
| | 7.507 (2.09), 7.738 (16.00), 8.160 (9.84), 8.723 (1.47), 12.243 (2.21). | |
| 398 | 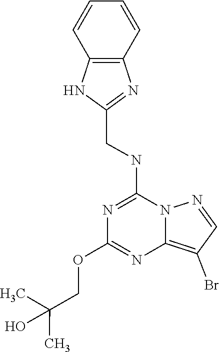 | 58% LC-MS (Method 2): Rₜ = 0.97 min; MS (ESIpos): m/z = 432 [M + H]⁺ |
| | 1-[(4-{[(1H-benzimidazol-2-yl)methyl]amino}-8- | |
| | bromopyrazolo[1,5-a][1,3,5]triazin-2-yl)oxy]-2-methylpropan-2-ol | |
| | ¹H-NMR (400 MHz,DMSO-d6) δ [ppm]: 1.130 (16.00), 1.232 (0.20), | |
| | 2.331 (0.22), 2.518 (1.28), 2.523 (0.80), 2.673 (0.22), 4.047 (4.82), | |
| | 4.672 (1.84), 4.928 (2.45), 7.134 (0.93), 7.148 (1.00), 7.401 (0.38), | |
| | 7.418 (0.37), 7.538 (0.40), 7.554 (0.34), 8.236 (5.26), 9.488 (0.39), | |
| | 12.290 (0.46). | |
| 399 | 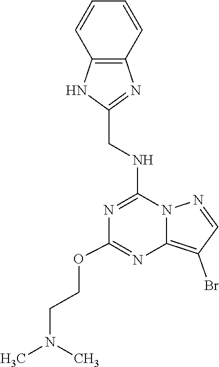 [SMILES: CN(C)CCOc1nc(NCc2nc3ccccc3[nH]2)n2ncc(Br)c2n1] | 50% LC-MS (Method 2): Rₜ = 1.01 min; MS (ESIpos): m/z = 431 [M + H]⁺ |
| | N-[(1H-benzimidazol-2-yl)methyl]-8-bromo-2-[2- | |
| | (dimethylamino)ethoxy]pyrazolo[1,5-a][1,3,5]triazin-4-amine | |
| | ¹H-NMR (400 MHz, DMSO-d6) δ [ppm]: 2.120 (16.00), 2.517 (2.30), | |
| | 2.522 (0.82), 2.531 (0.89), 4.305 (0.93), 4.319 (1.76), 4.334 (0.92), | |
| | 4.903 (1.07), 7.115 (0.18), 7.125 (0.94), 7.133 (0.91), 7.140 (0.93), | |
| | 7.148 (1.05), 7.158 (0.21), 7.467 (0.20), 8.236 (4.10), 9.516 (0.25). | |
| 400 | 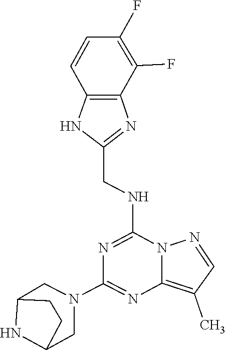 [SMILES: Cc1cnn2c(NCc3nc4c(F)c(F)ccc4[nH]3)nc(N3CC4CCC(C3)N4)nc12] | 47% LC-MS (Method 1): Rₜ = 0.74 min; MS (ESIneg): m/z = 424 [M − H]⁻ |
| | 2-(3,8-diazabicyclo[3.2.1]octan-3-yl)-N-[(4, 5-difluoro-1H- | |
| | benzimidazol-2-yl)methyl]-8-methylpyrazolo[1,5-a][1,3,5]triazin- | |
| | 4-amine | |
| | ¹H-NMR (400 MHz,DMSO-d6) δ [ppm]: 1.446 (0.44), 2.007 (16.00), | |
| | 2.332 (0.77), 2.518 (3.44), 2.523 (2.34), 2.539 (0.54), 2.829 (0.84), | |
| | 2.857 (0.88), 4.142 (0.54), 4.805 (2.49), 7.141 (0.49), 7.163 (0.93), | |
| | 7.169 (0.57), 7.181 (0.99), 7.192 (1.02), 7.209 (1.19), 7.225 (0.72), | |
| | 7.753 (6.00), 8.259 (7.79), 8.893 (0.62). | |
| 401 | 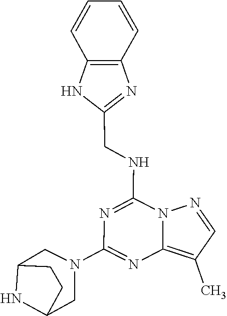 [SMILES: Cc1cnn2c(NCc3nc4ccccc4[nH]3)nc(N3CC4CCC(C3)N4)nc12] | 47% LC-MS (Method 2): Rₜ = 0.88 min; MS (ESIpos): m/z = 390 [M + H]⁺ |
| | N-[(1H-benzimidazol-2-yl)methyl]-2-(3,8- | |
| | diazabicyclo[3.2.1]octan-3-yl)-8-methylpyrazolo[1,5- | |
| | a][1,3,5]triazin-4-amine | |
| | ¹H-NMR (400 MHz, DMSO-d6) δ [ppm]: 1.232 (0.49), 1.395 (0.59), | |
| | 1.751 (0.31), 2.007 (16.00), 2.332 (0.39), 2.336 (0.19), 2.518 (1.83), | |
| | 2.523 (1.25), 2.539 (1.65), 2.678 (0.21), 2.769 (1.03), 2.800 (1.04), | |
| | 4.110 (0.65), 4.812 (3.39), 7.109 (1.75), 7.123 (1.90), 7.389 (0.63), | |
| | 7.402 (0.63), 7.514 (0.70), 7.731 (5.88), 8.752 (0.39), 12.243 (0.82). | |
| 402 | 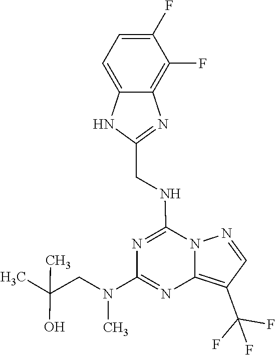 [SMILES: CN(CC(C)(C)O)c1nc(NCc2nc3c(F)c(F)ccc3[nH]2)n2ncc(C(F)(F)F)c2n1] | 34% LC-MS (Method 1): Rₜ = 1.16 min; MS (ESIpos): m/z = 471 [M + H]⁺ |
| | 1-{[4-{[(4,5-difluoro-1H-benzimidazol-2-yl)methyl]amino}-8- | |
| | (trifluoromethyl)pyrazolo[1,5-a][1,3,5]triazin-2- | |
| | yl](methyl)amino}-2-methylpropan-2-ol | |
| | ¹H-NMR (400 MHz, DMSO-d6) δ [ppm]: 0.707 (8.00), 0.852 (0.43), | |
| | 1.066 (4.89), 1.099 (0.53), 1.154 (3.89), 1.173 (8.24), 1.190 (4.35), | |
| | 1.233 (1.31), 1.988 (16.00), 2.336 (0.81), 2.518 (9.85), 2.523 (7.64), | |
| | 2.678 (0.80), 3.033 (2.93), 3.122 (10.18), 3.389 (3.29), 3.556 (1.37), | |
| | 4.000 (1.11), 4.017 (3.37), 4.035 (3.30), 4.053 (1.04), 4.350 (1.01), | |
| | 4.669 (1.06), 4.812 (1.87), 4.825 (1.86), 4.908 (0.66), 4.920 (0.64), | |
| | 5.759 (0.79), 7.174 (1.20), 7.186 (1.34), 7.199 (0.80), 7.221 (0.57), | |
| | 8.227 (1.22), 8.235 (3.40), 9.223 (0.54), 12.661 (0.52). | |
| 403 | 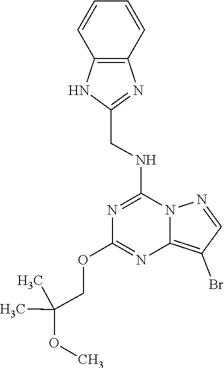 [SMILES: COC(C)(C)COc1nc(NCc2nc3ccccc3[nH]2)n2ncc(Br)c2n1] | 24% LC-MS (Method 2): Rₜ = 1.11 min; MS (ESIpos): m/z = 446 [M + H]⁺ |
| | N-[(1H-benzimidazol-2-yl)methyl]-8-bromo-2-(2-methoxy-2- | |
| | methylpropoxy)pyrazolo[1,5-a][1,3,5]triazin-4-amine | |
| | ¹H-NMR (400 MHz, DMSO-d6) δ [ppm]: 1.125 (16.00), 1.232 (0.19), | |
| | 2.331 (0.21), 2.518 (1.19), 2.523 (0.72), 2.540 (0.45), 2.673 (0.21), | |
| | 3.069 (13.02), 4.155 (4.81), 4.926 (3.38), 7.132 (0.91), 7.146 (0.97), | |
| | 7.399 (0.33), 7.536 (0.35), 8.238 (4.54), 9.478 (0.18), 12.302 (0.34). | |
| 404 | 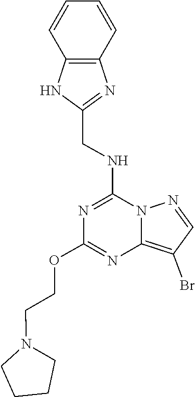 [SMILES: Brc1cnn2c(NCc3nc4ccccc4[nH]3)nc(OCCN3CCCC3)nc12] | 37% LC-MS (Method 2): Rₜ = 1.10 min; MS (ESIpos): m/z = 457 [M + H]⁺ |
| | N-[(1H-benzimidazol-2-yl)methyl]-8-bromo-2-[2-(pyrrolidin-1- | |
| | yl)ethoxy]pyrazolo[1,5-a][1,3,5]triazin-4-amine | |
| | ¹H-NMR (400 MHz, DMSO-d6) δ [ppm]: 1.232 (0.55), 1.586 (3.16), | |
| | 1.594 (4.88), 1.603 (9.47), 1.612 (4.86), 1.619 (3.29), 2.323 (0.51), | |
| | 2.327 (0.70), 2.331 (0.52), 2.383 (3.62), 2.386 (3.66), 2.399 (8.31), | |
| | 2.415 (3.20), 2.518 (2.63), 2.523 (1.68), 2.540 (0.62), 2.655 (3.41), | |
| | 2.670 (7.54), 2.684 (3.48), 4.313 (3.44), 4.329 (6.76), 4.343 (3.31), | |
| | 4.904 (6.54), 7.131 (2.64), 7.142 (2.78), 7.405 (0.79), 7.531 (0.81), | |
| | 8.235 (16.00), 9.519 (1.15), 12.283 (0.91). | |
| 405 | 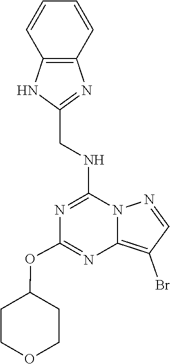 [SMILES: Brc1cnn2c(NCc3nc4ccccc4[nH]3)nc(OC3CCOCC3)nc12] | 73% LC-MS (Method 2): Rₜ = 1.01 min; MS (ESIpos): m/z = 444 [M + H]⁺ |
| | N-[(1H-benzimidazol-2-yl)methyl]-8-bromo-2-[(oxan-4- | |
| | yl)oxy]pyrazolo[1,5-a][1,3,5]triazin-4-amine | |
| | ¹H-NMR (400 MHz, DMSO-d6) δ [ppm]: 1.103 (16.00), 1.489 (0.45), | |
| | 1.499 (0.55), 1.513 (0.91), 1.522 (1.40), 1.531 (0.80), 1.536 (0.77), | |
| | 1.545 (1.48), 1.555 (1.06), 1.569 (0.65), 1.579 (0.55), 1.850 (1.28), | |
| | 1.860 (1.36), 1.883 (1.15), 1.892 (1.10), 2.518 (2.54), 2.523 (1.57), | |
| | 3.073 (4.64), 3.357 (2.65), 3.381 (1.41), 3.387 (1.16), 3.700 (0.98), | |
| | 3.711 (2.15), 3.721 (1.20), 3.729 (1.08), 3.740 (1.88), 3.751 (0.85), | |
| | 4.900 (4.64), 5.004 (0.59), 5.016 (0.80), 5.026 (1.12), 5.038 (0.80), | |
| | 5.049 (0.56), 7.130 (1.88), 7.144 (2.05), 7.152 (1.31), 7.393 (0.75), | |
| | 7.410 (0.73), 7.532 (0.80), 7.547 (0.71), 8.137 (0.60), 8.236 (11.17), | |
| | 9.546 (0.68), 12.302 (0.94). | |
| 406 | 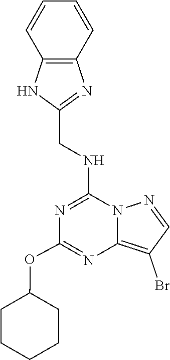 [SMILES: Brc1cnn2c(NCc3nc4ccccc4[nH]3)nc(OC3CCCCC3)nc12] | 36% LC-MS (Method 2): Rₜ = 1.33 min; MS (ESIpos): m/z = 442 [M + H]⁺ |
| | N-[(1H-benzimidazol-2-yl)methyl]-8-bromo-2- | |
| | (cyclohexyloxy)pyrazolo[1,5-a][1,3,5]triazin-4-amine | |
| | ¹H-NMR (400 MHz, DMSO-d6) δ [ppm]: 0.850 (0.52), 1.156 (1.05), | |
| | 1.182 (1.50), 1.229 (3.36), 1.247 (2.83), 1.279 (2.72), 1.305 (1.46), | |
| | 1.322 (1.53), 1.352 (3.09), 1.378 (2.47), 1.403 (1.02), 1.462 (1.39), | |
| | 1.595 (2.60), 1.616 (2.39), 1.843 (2.65), 2.074 (5.94), 2.285 (0.47), | |
| | 2.331 (1.72), 2.518 (10.79), 2.523 (6.37), 2.539 (1.59), 2.673 (1.71), | |
| | 4.841 (1.14), 4.854 (1.62), 4.864 (2.42), 4.888 (16.00), 7.110 (1.19), | |
| | 7.120 (6.45), 7.128 (6.25), 7.135 (6.52), 7.143 (7.29), 7.153 (1.44), | |
| | 7.467 (2.23), 8.199 (10.56), 8.544 (1.43). | |
| 407 | 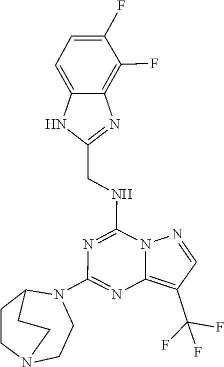 [SMILES: Fc1ccc2[nH]c(CNc3nc(N4CCN5CCC4CC5)nc4c(C(F)(F)F)cnn34)nc2c1F] | 42% LC-MS (Method 2): Rₜ = 1.12 min; MS (ESIpos): m/z = 494 [M + H]⁺ |
| | 2-(1,4-diazabicyclo[3.2.2]nonan-4-yl)-N-[(4, 5-difluoro-1H- | |
| | benzimidazol-2-yl)methyl]-8-(trifluoromethyl)pyrazolo[1,5- | |
| | a][1,3,5]triazin-4-amine | |
| | ¹H-NMR (400 MHz, DMSO-d6) δ [ppm]: 0.851 (0.59), 1.035 (5.62), | |
| | 1.052 (14.24), 1.070 (6.66), 1.154 (1.96), 1.172 (4.11), 1.190 (2.16), | |
| | 1.232 (2.35), 1.336 (1.76), 1.347 (1.83), 1.567 (1.24), 1.603 (2.02), | |
| | 1.614 (2.35), 1.629 (2.42), 1.640 (2.35), 1.752 (0.78), 1.769 (0.65), | |
| | 1.891 (2.16), 1.949 (1.50), 1.988 (7.25), 2.318 (1.24), 2.323 (2.87), | |
| | 2.327 (4.11), 2.331 (2.87), 2.337 (1.24), 2.518 (16.00), 2.523 (11.36), | |
| | 2.537 (12.02), 2.660 (1.83), 2.665 (3.79), 2.669 (5.16), 2.673 (4.11), | |
| | 2.678 (2.74), 2.707 (3.40), 2.767 (1.96), 2.796 (4.18), 2.803 (4.31), | |
| | 2.815 (8.49), 2.824 (6.53), 2.830 (7.12), 2.850 (2.68), 2.858 (2.35), | |
| | 2.871 (2.55), 2.881 (2.16), 2.893 (2.48), 2.907 (1.57), 3.194 (0.85), | |
| | 3.206 (0.72), 3.265 (0.65), 3.282 (0.91), 3.424 (0.91), 3.441 (0.91), | |
| | 3.790 (2.55), 3.878 (0.52), 3.927 (2.29), 3.941 (3.59), 3.999 (0.65), | |
| | 4.017 (1.50), 4.035 (1.44), 4.053 (0.46), 4.384 (1.89), 4.559 (1.37), | |
| | 4.843 (11.04), 7.138 (0.52), 7.160 (1.37), 7.177 (2.02), 7.189 (2.02), | |
| | 7.204 (3.27), 7.219 (2.81), 8.123 (0.46), 8.185 (0.59), 8.216 (5.16), | |
| | 8.223 (7.18). | |
| 408 | 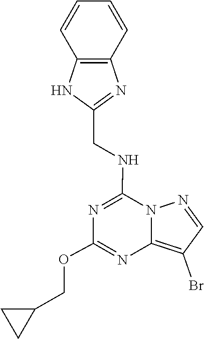 [SMILES: Brc1cnn2c(NCc3nc4ccccc4[nH]3)nc(OCC3CC3)nc12] | 66% LC-MS (Method 2): Rₜ = 1.16 min; MS (ESIpos): m/z = 414 [M + H]⁺ |
| | N-[(1H-benzimidazol-2-yl)methyl]-8-bromo-2- | |
| | (cyclopropylmethoxy)pyrazolo[1,5-a][1,3,5]triazin-4-amine | |
| | ¹H-NMR (400 MHz, DMSO-d6) δ [ppm]: 0.268 (1.31), 0.279 (5.20), | |
| | 0.283 (4.62), 0.291 (5.08), 0.295 (5.03), 0.305 (1.70), 0.460 (1.64), | |
| | 0.471 (4.23), 0.475 (4.36), 0.480 (2.38), 0.491 (4.53), 0.495 (4.21), | |
| | 0.506 (1.33), 1.103 (11.85), 1.120 (0.46), 1.126 (0.69), 1.139 (1.20), | |
| | 1.146 (1.14), 1.158 (1.82), 1.166 (0.99), 1.170 (1.10), 1.177 (1.15), | |
| | 1.189 (0.60), 1.196 (0.42), 2.523 (2.44), 3.073 (3.64), 4.066 (9.41), | |
| | 4.085 (9.14), 4.904 (9.30), 7.132 (3.31), 7.146 (3.51), 7.413 (1.09), | |
| | 7.535 (1.18), 8.227 (16.00), 9.496 (0.91), 12.300 (1.09). | |
| 409 | 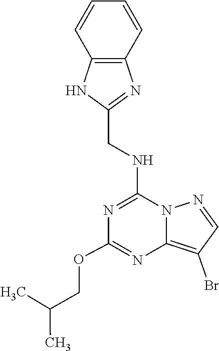 [SMILES: CC(C)COc1nc(NCc2nc3ccccc3[nH]2)n2ncc(Br)c2n1] | 30% LC-MS (Method 2): Rₜ = 1.43 min; MS (ESIpos): m/z = 416 [M + H]⁺ |
| | N-[(1H-benzimidazol-2-yl)methyl]-8-bromo-2-(2- | |
| | methylpropoxy)pyrazolo[1,5-a][1,3,5]triazin-4-amine | |
| | ¹H-NMR (400 MHz, DMSO-d6) δ [ppm]: 0.884 (15.53), 0.901 (16.00), | |
| | 1.914 (0.50), 1.931 (0.98), 1.948 (1.21), 1.964 (0.95), 1.981 (0.47), | |
| | 4.011 (4.89), 4.028 (4.76), 4.912 (7.12), 7.133 (1.87), 7.147 (1.99), | |
| | 7.398 (0.72), 7.411 (0.71), 7.533 (0.75), 8.229 (6.90), 12.283 (0.51). | |
| 410 | 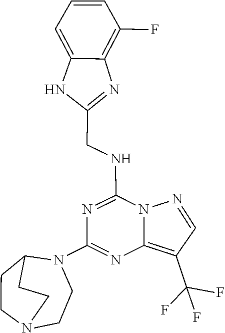 [SMILES: Fc1cccc2[nH]c(CNc3nc(N4CCN5CCC4CC5)nc4c(C(F)(F)F)cnn34)nc12] | 26% LC-MS (Method 2): Rₜ = 1.09 min; MS (ESIpos): m/z = 476 [M + H]⁺ |
| | 2-(1,4-diazabicyclo[3.2.2]nonan-4-yl)-N-[(4-fluoro-1H- | |
| | benzimidazol-2-yl)methyl]-8-(trifluoromethyl)pyrazolo[1, 5- | |
| | a][1,3,5]triazin-4-amine | |
| | ¹H-NMR (400 MHz, DMSO-d6) δ [ppm]: 0.851 (0.76), 1.232 (4.15), | |
| | 1.353 (2.43), 1.603 (1.68), 1.633 (2.03), 1.666 (2.06), 1.949 (1.60), | |
| | 2.074 (4.82), 2.318 (1.23), 2.323 (2.67), 2.327 (3.80), 2.331 (2.71), | |
| | 2.337 (1.24), 2.518 (16.00), 2.523 (11.27), 2.665 (3.75), 2.669 (5.14), | |
| | 2.673 (4.19), 2.678 (2.89), 2.704 (3.93), 2.762 (2.22), 2.786 (2.68), | |
| | 2.820 (5.37), 2.867 (1.90), 3.801 (2.87), 3.930 (2.62), 3.944 (4.23), | |
| | 4.400 (2.25), 4.563 (1.66), 4.856 (10.05),6.935 (1.83), 7.083 (0.95), | |
| | 7.103 (2.36), 7.115 (3.13), 7.231 (1.89), 8.220 (8.30), 9.215 (1.40), | |
| | 12.559 (1.49). | |
| 411 | 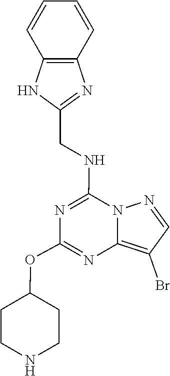 [SMILES: Brc1cnn2c(NCc3nc4ccccc4[nH]3)nc(OC3CCNCC3)nc12] | 20% LC-MS (Method 2): Rₜ = 0.94 min; MS (ESIpos): m/z = 443 [M + H]⁺ |
| | N-[(1H-benzimidazol-2-yl)methyl]-8-bromo-2-[(piperidin-4- | |
| | yl)oxy]pyrazolo[1,5-a][1,3,5]triazin-4-amine | |
| | ¹H-NMR (400 MHz, DMSO-d6) δ [ppm]: 1.231 (1.69), 1.343 (0.66), | |
| | 1.353 (0.89), 1.377 (1.86), 1.399 (1.95), 1.424 (0.92), 1.433 (0.77), | |
| | 1.751 (1.92), 1.812 (2.20), 1.822 (1.61), 1.834 (1.93), 1.843 (1.79), | |
| | 1.884 (0.76), 2.421 (1.42), 2.427 (1.65), 2.452 (3.36), 2.518 (4.22), | |
| | 2.523 (2.66), 2.540 (0.81), 2.815 (2.89), 2.824 (1.69), 2.836 (1.51), | |
| | 2.846 (2.48), 4.875 (1.13), 4.894 (12.68), 4.909 (1.55), 4.923 (0.91), | |
| | 4.933 (0.47), 7.124 (3.05), 7.131 (3.31), 7.139 (3.44), 7.146 (3.40), | |
| | 7.411 (0.72), 7.523 (0.72), 8.222 (16.00), 8.482 (0.87), 12.297 (0.41). | |
| 412 | 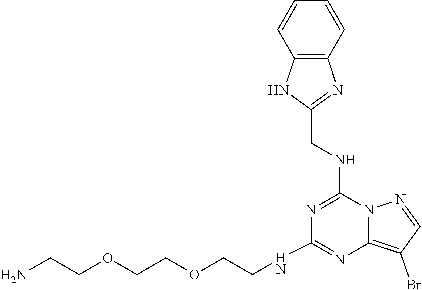 [SMILES: NCCOCCOCCNc1nc(NCc2nc3ccccc3[nH]2)n2ncc(Br)c2n1] | 16% LC-MS (Method 1): Rₜ = 0.59 min; MS (ESIneg): m/z = 488 [M − H]⁻ |
| | N2-{2-[2-(2-aminoethoxy)ethoxy]ethyl}-N4-[(1H-benzimidazol-2- | |
| | yl)methyl]-8-bromopyrazolo[1,5-a][1,3,5]triazine-2,4-diamine | |
| | ¹H-NMR (400 MHz, DMSO-d6) δ [ppm]: 2.318 (0.56), 2.323 (1.29), | |
| | 2.327 (1.80), 2.332 (1.27), 2.336 (0.56), 2.518 (7.73), 2.523 (5.30), | |
| | 2.660 (0.62), 2.665 (1.33), 2.669 (1.86), 2.673 (1.30), 2.679 (0.61), | |
| | 2.748 (2.21), 2.761 (2.70), 3.159 (1.97), 3.257 (1.62), 3.303 (2.07), | |
| | 3.359 (2.34), 3.523 (5.18), 3.537 (5.72), 3.624 (0.61), 4.857 (5.23), | |
| | 5.759 (2.34), 7.121 (2.98), 7.129 (3.07), 7.136 (3.09), 7.143 (2.99), | |
| | 7.263 (1.00), 7.381 (0.43), 7.463 (3.27), 7.471 (3.06), 7.478 (2.95), | |
| | 7.485 (2.70), 7.908 (0.60), 7.991 (16.00), 8.409 (11.16). | |

### Example 413

### N-[(4,5-difluoro-1H-benzimidazol-2-yl)methyl]-2-(morpholin-4-yl)-8-(1,3-thiazol-2-yl)pyrazolo [1,5-a][1,3,5]triazin-4-amine

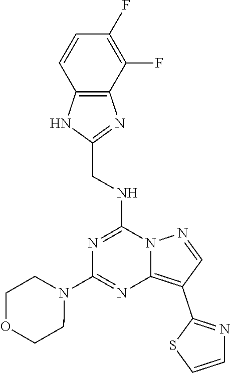 [SMILES: Fc1ccc2[nH]c(CNc3nc(N4CCOCC4)nc4c(-c5nccs5)cnn34)nc2c1F]

A solution of N-[(4,5-difluoro-1-{[2-(trimethylsilyl)ethoxy]methyl}-1H-benzimidazol-2-yl)methyl]-2-(morpholin-4-yl)-8-(1,3-thiazol-2-yl)pyrazolo[1,5-a][1,3,5]triazin-4-amine (Intermediate 233, 730 mg, 71% purity, 0.864 mmol) in trifluoroacetic acid (5.0 mL) and dichloromethane (5.0 mL) was stirred at room temperature for 16 hours. The reaction mixture was concentrated to give a residue. The residue was purified by preparative HPLC [Instrument: ACSWH-GX-K; Column: Phenomenex Gemini-NX C18 75*30 mm*3 μm; eluent A: water (0.1% trifluoroacetic acid), eluent B: acetonitrile; gradient: 0-7 min 28-38% B; flow 25 mL/min; temperature: RT; Detector: UV 220/254 nm] to give N-[(4,5-difluoro-1H-benzimidazol-2-yl)methyl]-2-(morpholin-4-yl)-8-(1,3-thiazol-2-yl)pyrazolo[1,5-a][1,3,5]triazin-4-amine (158 mg, 99% purity, 39% yield) as a white solid.

LC-MS (Method D): Rₜ=0.693 min; MS (ESIpos): m/z=470.2 [M+H]⁺.

¹H-NMR (400 MHz, DMSO-d₆): 5 [ppm]=9.34 (t, J=5.6 Hz, 1H), 8.46 (s, 1H), 7.79 (d, J=3.2 Hz, 1H), 7.58 (d, J=3.2 Hz, 1H), 7.33-7.16 (m, 2H), 4.93 (d, J=5.6 Hz, 2H), 3.87-3.50 (m, 8H).

### Example 414

### N-[(4-methoxy-1H-benzimidazol-2-yl)methyl]-2-(morpholin-4-yl)-8-(1,3-thiazol-2-yl)pyrazolo [1,5-a][1,3,5]triazin-4-amine

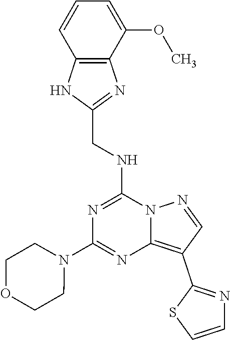 [SMILES: COc1cccc2[nH]c(CNc3nc(N4CCOCC4)nc4c(-c5nccs5)cnn34)nc12]

A solution of N-[(4-methoxy-1-{[2-(trimethylsilyl)ethoxy]methyl}-1H-benzimidazol-2-yl)methyl]-2-(morpholin-4-yl)-8-(1,3-thiazol-2-yl)pyrazolo[1,5-a][1,3,5]triazin-4-amine (Intermediate 234, 400 mg, 67% purity, 0.451 mmol) in trifluoroacetic acid (5.0 mL) and dichloromethane (5.0 mL) was stirred at room temperature for 16 hours. The reaction mixture was concentrated to give a residue and purified by preparative HPLC [Instrument: CASWH-Prep-NPLC-B; Column: Welch Ultimate XB-NH2 250*50*10 μm; eluent A: heptane, eluent B: ethanol (0.1% ammonia water); gradient: 0-10 min 30-70% B; flow 100 mL/min; temperature: RT; Detector: UV 220/254 nm] to give N-[(4-methoxy-1H-benzimidazol-2-yl)methyl]-2-(morpholin-4-yl)-8-(1,3-thiazol-2-yl)pyrazolo[1,5-a][1,3,5]triazin-4-amine (88.4 mg, 99% purity, 42% yield) as a white solid.

LC-MS (Method C): Rₜ=0.656 min; MS (ESIpos): m/z=464.2 [M+H]⁺.

¹H-NMR (400 MHz, DMSO-d₆): 5 [ppm]=12.77-12.12 (m, 1H), 9.20 (s, 1H), 8.42 (s, 1H), 7.74 (d, J=3.2 Hz, 1H), 7.55 (d, J=3.2 Hz, 1H), 7.06 (t, J=7.6 Hz, 2H), 6.70 (s, 1H), 4.99 (d, J=8.0 Hz, 2H), 3.90 (s, 3H), 3.85-3.65 (br. s, 4H), 3.64-3.38 (m, 4H).

### Example 415

### N-[(4,5-difluoro-1H-benzimidazol-2-yl)methyl]-2-(4-methylpiperazin-1-yl)-8-(1,3-thiazol-2-yl)pyrazolo[1,5-a][1,3,5]triazin-4-amine

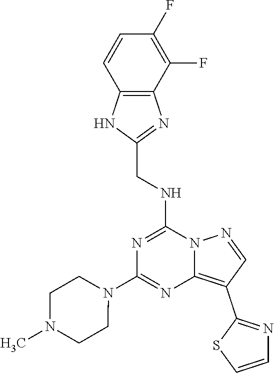 [SMILES: CN1CCN(c2nc(NCc3nc4c(F)c(F)ccc4[nH]3)n3ncc(-c4nccs4)c3n2)CC1]

A solution of N-[(4,5-difluoro-1-{[2-(trimethylsilyl)ethoxy]methyl}-1H-benzimidazol-2-yl)methyl]-N-[(4-methoxyphenyl)methyl]-2-(4-methylpiperazin-1-yl)-8-(1,3-thiazol-2-yl)pyrazolo[1,5-a][1,3,5]triazin-4-amine (Intermediate 239, 50.0 mg, 60% purity, 0.04 mmol) in trifluoroacetic acid (5.0 mL) was stirred at 80° C. for 16 hours. The reaction mixture was concentrated and purified by preparative HPLC [Instrument: ACSWH-GX-C; Column: Phenomenex luna C18 150*25 mm*10 μm; eluent A: water (0.225% formic acid in water), eluent B: acetonitrile; gradient: 0-10 min 6-36% B; flow 25 mL/min; temperature: RT; Detector: UV 220/254 nm] to give N-[(4,5-difluoro-1H-benzimidazol-2-yl)methyl]-2-(4-methylpiperazin-1-yl)-8-(1,3-thiazol-2-yl)pyrazolo[1,5-a][1,3,5]triazin-4-amine (11.8 mg, 99% purity, 59% yield) as a white solid.

LC-MS (Method C): Rₜ=0.74 min; MS (ESIpos): m/z=483.3 [M+H]⁺.

¹H NMR (400 MHz, DMSO-d₆) δ [ppm]=9.27 (s, 1H), 8.42 (s, 1H), 8.28 (s, 1H), 7.74 (d, J=3.2 Hz, 1H), 7.55 (d, J=3.2 Hz, 1H), 7.29-7.15 (m, 2H), 4.91 (s, 2H), 3.85-3.60 (m, 8H), 2.14 (s, 3H).

### Example 416

### N-[(7-methoxy-1H-benzimidazol-2-yl)methyl]-2-(4-methylpiperazin-1-yl)-8-(1,3-thiazol-2-yl)pyrazolo[1,5-a][1,3,5]triazin-4-amine

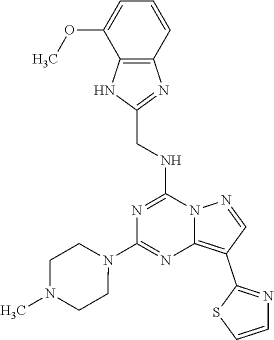 [SMILES: COc1cccc2nc(CNc3nc(N4CCN(C)CC4)nc4c(-c5nccs5)cnn34)[nH]c12]

A solution of N-[(4-methoxyphenyl)methyl]-N-[(7-methoxy-1-{[2-(trimethylsilyl)ethoxy]methyl}-1H-benzimidazol-2-yl)methyl]-2-(4-methylpiperazin-1-yl)-8-(1,3-thiazol-2-yl)pyrazolo[1,5-a][1,3,5]triazin-4-amine (Intermediate 241, 29.0 mg, 83% purity, 0.03 mmol) in trifluoroacetic acid (2.0 mL) was stirred at 80° C. for 16 hours. The mixture was concentrated and purified by preparative HPLC [Instrument: GX-T; Column:Phenomenex Synergi C18 150*25 mm*10 μm; eluent A: water (0.1% trifluoroacetic acid), eluent B: acetonitrile; gradient: 0-10 min 0-30% B; flow 25 mL/min; temperature: RT; Detector: UV 220/254 nm] to give N-((7-methoxy-1H-benzo[d]imidazol-2-yl)methyl)-2-(4-methylpiperazin-1-yl)-8-(thiazol-2-yl)pyrazolo[1,5-a][1,3,5]triazin-4-amine (10.8 mg, 97% purity, 67% yield) as a yellow solid.

LC-MS (Method A): Rₜ=0.80 min; MS (ESIpos): m/z=477.3 [M+H]⁺.

¹H NMR (400 MHz, CDCl₃) 5 [ppm]=10.16 (s, 1H), 9.49 (t, J=5.6 Hz, 1H), 8.51 (s, 1H), 7.78 (d, J=3.2 Hz, 1H), 7.60 (d, J=3.2 Hz, 1H), 7.40 (t, J=8.4 Hz, 1H), 7.29 (d, J=8.4 Hz, 1H), 7.05 (d, J=8.0 Hz, 1H), 5.17 (s, 2H), 4.65 (s, 4H), 3.98 (s, 3H), 3.24 (s, 4H), 2.79 (s, 3H).

### Example 417

### N-[(4,5-difluoro-1H-benzimidazol-2-yl)methyl]-2-(morpholin-4-yl)-8-(pyridazin-4-yl)pyrazolo[1,5-a][1,3,5]triazin-4-amine

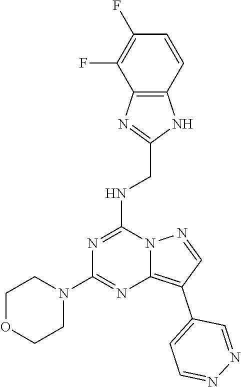 [SMILES: Fc1ccc2[nH]c(CNc3nc(N4CCOCC4)nc4c(-c5ccnnc5)cnn34)nc2c1F]

A solution of N-[(4,5-difluoro-1-{[2-(trimethylsilyl)ethoxy]methyl}-1H-benzimidazol-2-yl)methyl]-N-[(4-methoxyphenyl)methyl]-2-(morpholin-4-yl)-8-(pyridazin-4-yl)pyrazolo[1,5-a][1,3,5]triazin-4-amine (Intermediate 245, 150 mg, 90% purity, 0.19 mmol) in trifluoroacetic acid (1.8 mL, 23 mmol) was stirred at 80° C. for 3 hours. The reaction mixture was concentrated and purified by preparative HPLC [lnstrument:ACSWH-GX-N; Column: Phenomenex Synergi C18 150*25 mm*10 μm; eluent A: water (0.225% formic acid in water), eluent B: acetonitrile; gradient: 0-40 min 13-43% B; flow 25 mL/min; temperature: RT; Detector: UV 220/254 nm] to give N-[(4,5-difluoro-1H-benzimidazol-2-yl)methyl]-2-(morpholin-4-yl)-8-(pyridazin-4-yl)pyrazolo[1,5-a][1,3,5]triazin-4-amine (43.5 mg, 91% purity, 45% yield) as a yellow solid.

LC-MS (Method C): Rₜ=0.77 min; MS (ESIpos): m/z=464.9 [M+H]⁺.

¹H NMR (400 MHz, DMSO-d₆) 5 [ppm]=9.93 (s, 1H), 9.48 (t, J=5.6 Hz, 1H), 9.24-9.19 (m, 1H), 8.91 (s, 1H), 8.55 (dd, J=6.0, 2.4 Hz, 1H), 7.31-7.17 (m, 2H), 4.93 (d, J=5.6 Hz, 2H), 3.71-3.60 (m, 4H), 3.59-3.39 (m, 4H).

### Example 418

### N-[(4,5-difluoro-1H-benzimidazol-2-yl)methyl]-8-(1-methyl-1H-pyrazol-4-yl)-2-(morpholin-4-yl)pyrazolo[1,5-a][1,3,5]triazin-4-amine

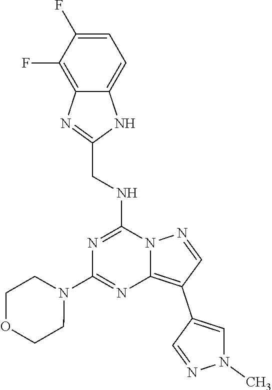 [SMILES: Cn1cc(-c2cnn3c(NCc4nc5c(F)c(F)ccc5[nH]4)nc(N4CCOCC4)nc23)cn1]

A solution of N-[(4,5-difluoro-1-{[2-(trimethylsilyl)ethoxy]methyl}-1H-benzimidazol-2-yl)methyl]-N-[(4-methoxyphenyl)methyl]-8-(1-methyl-1H-pyrazol-4-yl)-2-(morpholin-4-yl)pyrazolo[1,5-a] [1,3,5]triazin-4-amine (Intermediate 248, 108 mg, 92% purity, 0.139 mmol) in trifluoroacetic acid (3.0 mL) was stirred at 80° C. for 16 hours. The mixture was concentrated and purified by preparative HPLC [Instrument: ACSWH-GX-K; Column:Phenomenex Gemini-NX C18 75*30 mm*3 μm; eluent A: water (0.1% trifluoroacetic acid), eluent B: acetonitrile; gradient: 0-7 min 28-38% B; flow 25 mL/min; temperature: RT; Detector: UV 220/254 nm] to give N-[(4,5-difluoro-1H-benzimidazol-2-yl)methyl]-8-(1-methyl-1H-pyrazol-4-yl)-2-(morpholin-4-yl)pyrazolo [1,5-a][1,3,5]triazin-4-amine (47.5 mg, 99% purity, 73% yield) as a white solid.

LC-MS (Method D): Rₜ=0.73 min; MS (ESIpos): m/z=467.2 [M+H]⁺.

¹H NMR (400 MHz, DMSO-d₆) 5 [ppm]=9.05 (t, J=5.6 Hz, 1H), 8.21 (s, 1H), 8.00 (s, 1H), 7.83 (s, 1H), 7.28-7.17 (m, 2H), 4.90 (d, J=6.0 Hz, 2H), 3.87 (s, 3H), 3.66 (s, 4H), 3.53 (s, 4H).

### Example 419

### N-[(5,6-dichloro-1H-benzimidazol-2-yl)methyl]-8-(1-methyl-1H-pyrazol-4-yl)-2-(morpholin-4-yl)pyrazolo[1,5-a][1,3,5]triazin-4-amine

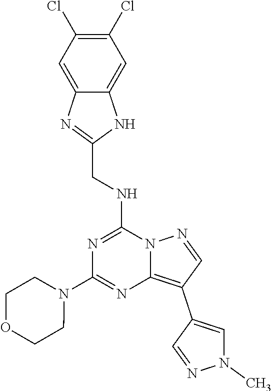 [SMILES: Cn1cc(-c2cnn3c(NCc4nc5cc(Cl)c(Cl)cc5[nH]4)nc(N4CCOCC4)nc23)cn1]

A solution of N-[(5,6-dichloro-1-{[2-(trimethylsilyl)ethoxy]methyl}-1H-benzimidazol-2-yl)methyl]-N-[(4-methoxyphenyl)methyl]-8-(1-methyl-1H-pyrazol-4-yl)-2-(morpholin-4-yl)pyrazolo[1,5-a] [1,3,5]triazin-4-amine (Intermediate 251, 157 mg, 91% purity, 0.191 mmol) in trifluoroacetic acid (4.0 mL) was stirred at 80° C. for 16 hours. The mixture was concentrated and purified by preparative HPLC [Instrument: ACSWH-GX-K; Column:Phenomenex Gemini-NX C18 75*30 mm*3 μm; eluent A: water (0.1% trifluoroacetic acid), eluent B: acetonitrile; gradient: 0-7 min 32-42% B; flow 25 mL/min; temperature: RT; Detector: UV 220/254 nm] to give N-[(5,6-dichloro-1H-benzimidazol-2-yl)methyl]-8-(1-methyl-1H-pyrazol-4-yl)-2-(morpholin-4-yl)pyrazolo [1,5-a][1,3,5]triazin-4-amine (38.1 mg, 99% purity, 40% yield) as a white solid.

LC-MS (Method C): Rₜ=0.86 min; MS (ESIpos): m/z=499.2 [M+H]⁺.

¹H NMR (400 MHz, DMSO-d₆) 5 [ppm]=9.06-9.04 (t, J=5.6 Hz, 1H), 8.21 (s, 1H), 8.00 (s, 1H), 7.83 (s, 1H), 7.78 (s, 2H), 4.91-4.89 (d, J=2.0 Hz, 2H), 3.86 (s, 3H), 3.64 (s, 4H), 3.53 (s, 4H).

### Example 420

### N-[(5,6-dichloro-1H-benzimidazol-2-yl)methyl]-2-(4-methylpiperazin-1-yl)-8-(1-methyl-1H-pyrazol-4-yl)pyrazolo[1,5-a][1,3,5]triazin-4-amine

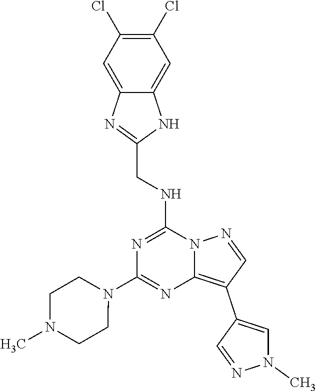

A solution of N-[(5,6-dichloro-1-{[2-(trimethylsilyl)ethoxy]methyl}-1H-benzimidazol-2-yl)methyl]-N-[(4-methoxyphenyl)methyl]-2-(4-methylpiperazin-1-yl)-8-(1-methyl-1H-pyrazol-4-yl)pyrazolo[1,5-a][1,3,5]triazin-4-amine (Intermediate 256, 97.2 mg, 78% purity, 0.1 mmol) in trifluoroacetic acid (2.0 mL) was stirred at 80° C. for 16 hours. The reaction mixture was concentrated to give a residue. The residue was diluted with methanol, adjusted to pH=7 with saturated sodium bicarbonate aqueous solution. The solution was concentrated and purified by preparative HPLC [Instrument: GX-D; Column: Phenomenex Gemini-NX C18 75*30 mm*3 μm; eluent A:10 mM ammonium bicarbonate in water, eluent B: acetonitrile; gradient: 0-8 min 22-52% B; flow 30 mL/min; temperature: room temperature; Detector: UV 220/254 nm] to give N-[(5,6-dichloro-1H-benzimidazol-2-yl)methyl]-2-(4-methylpiperazin-1-yl)-8-(1-methyl-1H-pyrazol-4-yl)pyrazolo[1,5-a][1,3,5]triazin-4-amine (12.2 mg, 98% purity, 23% yield) as a white solid.

LC-MS (Method A): Rₜ=0.94 min; MS (ESIpos): m/z=512.3 [M+H]⁺.

¹H NMR (400 MHz, DMSO-d₆) 5 [ppm]=9.08 (s, 1H), 8.30 (s, 1H), 8.09 (s, 1H), 7.93 (s, 1H), 7.85 (s, 2H), 4.98 (s, 2H), 3.98 (s, 3H), 3.75 (s, 4H), 2.30 (s, 4H), 2.24 (s, 3H).

### Example 421

### N-[(1H-benzimidazol-2-yl)methyl]-8-[1-(difluoromethyl)-1H-pyrazol-4-yl]-2-(morpholin-4-yl)pyrazolo[1,5-a][1,3,5]triazin-4-amine

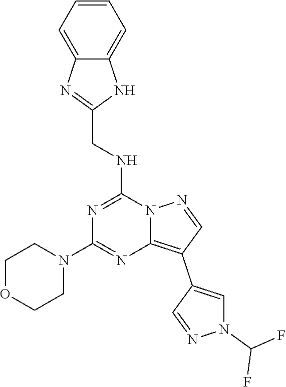 [SMILES: FC(F)n1cc(-c2cnn3c(NCc4nc5ccccc5[nH]4)nc(N4CCOCC4)nc23)cn1]

A solution of 8-[1-(difluoromethyl)-1H-pyrazol-4-yl]-N-[(4-methoxyphenyl)methyl]-2-(morpholin-4-yl)-N-[(1-{[2-(trimethylsilyl)ethoxy]methyl}-1H-benzimidazol-2-yl)methyl]pyrazolo[1,5-a][1,3,5]triazin-4-amine (Intermediate 260, 150 mg, 0.176 mmol) in trifluoroacetic acid (3.0 mL) was stirred at 60° C. for 16 hours. The solution was concentrated, diluted with methanol. After adjusting to pH=7 by sodium bicarbonate powder, the mixture was filtered. The filtrate was concentrated and purified by preparative HPLC [Instrument: ACSWH-GX-C; Column:Phenomenex Luna C18 150*25 mm*10 μm; eluent A: water (0.225% formic acid), eluent B: acetonitrile; gradient: 0-10 min 14-44% B; flow 25 mL/min; temperature: room temperature; Detector: UV 220/254 nm] to give N-(1H-benzimidazol-2-ylmethyl)-8-[1-(difluoromethyl)-1H-pyrazol-4-yl]-2-(morpholin-4-yl)pyrazolo[1,5-a][1,3,5]triazin-4-amine (59.4 mg, 99% purity, 72% yield) as a white solid.

LC-MS (Method C): Rₜ=0.79 min; MS (ESIpos): m/z=467.3 [M+H]⁺.

¹H NMR (400 MHz, DMSO-d₆) δ [ppm]=9.07 (t, J=6.0 Hz, 1H), 8.47 (s, 1H), 8.34 (s, 1H), 8.26 (s, 1H), 7.85 (t, J=59.6 Hz, 1H), 7.61-7.34 (m, 2H), 7.14 (dd, J=6.0, 3.2 Hz, 2H), 4.90 (d, J=5.6 Hz, 2H), 3.68 (s, 4H), 3.54 (s, 4H).

### Example 422

### N-[(4,5-difluoro-1H-benzimidazol-2-yl)methyl]-8-[1-(difluoromethyl)-1H-pyrazol-4-yl]-2-(morpholin-4-yl)pyrazolo[1,5-a][1,3,5]triazin-4-amine

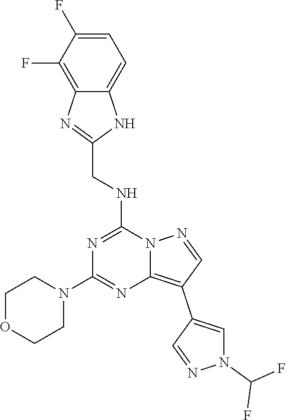 [SMILES: Fc1ccc2[nH]c(CNc3nc(N4CCOCC4)nc4c(-c5cnn(C(F)F)c5)cnn34)nc2c1F]

A solution of 8-[1-(difluoromethyl)-1H-pyrazol-4-yl]-N-[(4,5-difluoro-1-{[2-(trimethylsilyl)ethoxy] methyl}-1H-benzimidazol-2-yl)methyl]-N-[(4-methoxyphenyl)methyl]-2-(morpholin-4-yl)pyrazolo[1,5-a][1,3,5]triazin-4-amine (Intermediate 261, 160 mg, 0.20 mmol) in trifluoroacetic acid (3.0 mL) was stirred at 60° C. for 16 hours. The solution was concentrated, diluted with methanol. After adjusting to pH=7 by sodium bicarbonate powder, the mixture was filtered. The filtrate was concentrated and purified by preparative HPLC [Instrument: GX-P; Column: Phenomenex Synergi C18 150*25 mm*10 μm; eluent A: water (0.225% formic acid), eluent B: acetonitrile; gradient: 0-10 min 35-65% B; flow 25 mL/min; temperature: room temperature; Detector: UV 220/254 nm] to give N-[(4,5-difluoro-1H-benzimidazol-2-yl)methyl]-8-[1-(difluoromethyl)-1H-pyrazol-4-yl]-2-(morpholin-4-yl)pyrazolo[1,5-a][1,3,5]triazin-4-amine (43.4 mg, 99% purity, 43% yield) as a white solid.

LC-MS (Method C): Rₜ=0.87 min; MS (ESIpos): m/z=503.3 [M+H]⁺.

¹H NMR (400 MHz, DMSO-d₆) 5 [ppm]=9.30-8.98 (m, 1H), 8.47 (s, 1H), 8.34 (s, 1H), 8.26 (s, 1H), 7.85 (t, J=59.2 Hz, 1H), 7.29-7.13 (m, 2H), 4.89 (s, 2H), 3.67 (s, 4H), 3.53 (s, 4H).

### Example 423

### N-[(5,6-dichloro-1H-benzimidazol-2-yl)methyl]-8-[1-(difluoromethyl)-1H-pyrazol-4-yl]-2-(morpholin-4-yl)pyrazolo[1,5-a][1,3,5]triazin-4-amine

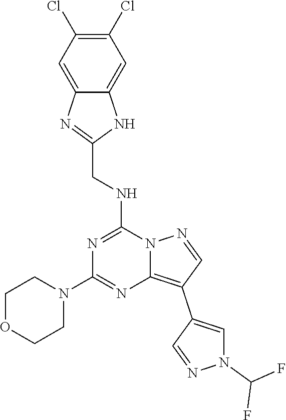 [SMILES: FC(F)n1cc(-c2cnn3c(NCc4nc5cc(Cl)c(Cl)cc5[nH]4)nc(N4CCOCC4)nc23)cn1]

A solution of N-[(5,6-dichloro-1-{[2-(trimethylsilyl)ethoxy]methyl}-1H-benzimidazol-2-yl)methyl]-8-[1-(difluoromethyl)-1H-pyrazol-4-yl]-N-(4-methoxybenzyl)-2-(morpholin-4-yl)pyrazolo[1,5-a][1,3,5]triazin-4-amine (Intermediate 262, 200 mg, 62% purity, 0.158 mmol) in trifluoroacetic acid (5 mL) was stirred at 60° C. for 16 hours. The solution was concentrated, diluted with methanol. After adjusting to pH=7 by sodium bicarbonate powder, the mixture was filtered. The filtrate was concentrated and purified by preparative HPLC [Instrument: GX-P; Column: Phenomenex Synergi C18 150*25 mm*10 μm; eluent A: water (0.225% formic acid), eluent B: acetonitrile; gradient: 0-10 min 41-71% B; flow 25 mL/min; temperature: room temperature; Detector: UV 220/254 nm] to give N-[(5,6-dichloro-1H-benzimidazol-2-yl)methyl]-8-[1-(difluoromethyl)-1H-pyrazol-4-yl]-2-(morpholin-4-yl)pyrazolo[1,5-a][1,3,5]triazin-4-amine (11.1 mg, 98% purity, 12.9% yield) as a white solid.

LC-MS (Method C): Rₜ=0.91 min; MS (ESIpos): m/z=535.3 [M+H]⁺.

¹H NMR (400 MHz, DMSO-d₆): δ [ppm]=9.12 (br. s, 1H), 8.47 (s, 1H), 8.34 (s, 1H), 8.25 (s, 1H), 8.02-7.65 (m, 3H), 4.88 (s, 2H), 3.66-3.53 (m, 8H).

### Example 424

### N-[(5,6-dichloro-1H-benzimidazol-2-yl)methyl]-2-(morpholin-4-yl)-8-(1,3-thiazol-4-yl)pyrazolo [1,5-a][1,3,5]triazin-4-amine

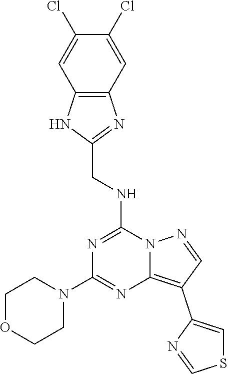 [SMILES: Clc1cc2nc(CNc3nc(N4CCOCC4)nc4c(-c5cscn5)cnn34)[nH]c2cc1Cl]

A solution of N-[(5,6-dichloro-1-{[2-(trimethylsilyl)ethoxy]methyl}-1H-benzimidazol-2-yl)methyl]-N-[(4-methoxyphenyl)methyl]-2-(morpholin-4-yl)-8-(1,3-thiazol-4-yl)pyrazolo[1,5-a][1,3,5]triazin-4-amine (Intermediate 265, 250 mg, 95% purity, 0.32 mmol) in trifluoroacetic acid (5.0 mL) was stirred at 80° C. for 3 hours. The solution was concentrated, diluted with methanol. After adjusting to pH=7 by sodium bicarbonate powder, the mixture was filtered. The filtrate was concentrated and purified by preparative HPLC [Instrument: ACSWH-GX-N; Column: Phenomenex Synergi C18 150*25 mm*10 μm; eluent A: water (0.1% trifluoroacetic acid), eluent B: acetonitrile; gradient: 0-40 min 40-70% B; flow 25 mL/min; temperature: room temperature; Detector: UV 220/254 nm] to give N-[(5,6-dichloro-1H-benzimidazol-2-yl)methyl]-2-(morpholin-4-yl)-8-(1,3-thiazol-4-yl)pyrazolo[1,5-a][1,3,5]triazin-4-amine (101.0 mg, 99% purity, 63% yield) as a brown solid.

LC-MS (Method C): Rₜ=0.88 min; MS (ESIpos): m/z=501.9 [M+H]⁺.

¹H NMR (400 MHz, DMSO-d₆) 5 [ppm]=9.23-9.14 (m, 1H), 9.12 (d, J=2.0 Hz, 1H), 8.36 (s, 1H), 7.87 (d, J=2.0 Hz, 1H), 7.81 (s, 2H), 4.93 (br d, J=4.8 Hz, 2H), 3.66 (s, 8H).

### Example 425

trifluoroacetic acid N-[(4,5-difluoro-1H-benzimidazol-2-yl)methyl]-8-(2,2-difluoroethyl)-2-(morpholin-4-yl)pyrazolo[1,5-a][1,3,5]triazin-4-amine

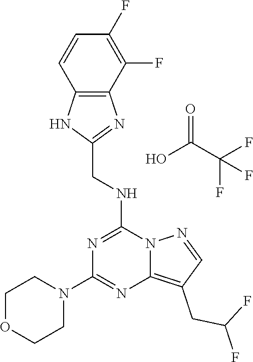 [SMILES: Fc1ccc2[nH]c(CNc3nc(N4CCOCC4)nc4c(CC(F)F)cnn34)nc2c1F ; O=C(O)C(F)(F)F]

A solution of 8-(2,2-difluoroethyl)-N-[(4,5-difluoro-1-{[2-(trimethylsilyl)ethoxy]methyl}-1H-benzimidazol-2-yl)methyl]-N-[(4-methoxyphenyl)methyl]-2-(morpholin-4-yl)pyrazolo[1,5-a][1,3,5]triazin-4-amine (Intermediate 268, 180 mg, 99% purity, 0.25 mmol) in trifluoroacetic acid (4.0 mL) at room temperature was stirred at 80° C. for 16 hours. The solution was concentrated, diluted with methanol. After adjusting to pH=7 by sodium bicarbonate powder, the mixture was filtered. The filtrate was concentrated and purified by preparative HPLC [Instrument: ACSWH-GX-N; Column: Phenomenex Synergi C18 150*25 mm*10 μm; eluent A: water (0.1% trifluoroacetic acid), eluent B: acetonitrile; gradient: 0-40 min 30-60% B; flow 25 mL/min; temperature: room temperature; Detector: UV 220/254 nm] to give N-[(4,5-difluoro-1H-benzimidazol-2-yl)methyl]-8-(2,2-difluoroethyl)-2-(morpholin-4-yl)pyrazolo[1,5-a][1,3,5]triazin-4-amine trifluoroacetate (94.4 mg, 99% purity, 65% yield) as a white solid.

LC-MS (Method C): Rₜ=0.84 min; MS (ESIpos): m/z=450.9 [M+H]⁺.

¹H NMR (400 MHz, DMSO-d₆) 5 [ppm]=9.14-9.02 (m, 1H), 7.87 (s, 1H), 7.31-7.16 (m, 2H), 6.21 (tt, J=56.8, 4.0 Hz, 1H), 4.92-4.85 (m, 2H), 3.64-3.54 (m, 4H), 3.53-3.40 (m, 4H), 3.05 (td, J=18.0, 4.0 Hz, 2H).

### Example 426

### N-[(4,5-difluoro-1H-benzimidazol-2-yl)methyl]-2-(morpholin-4-yl)-8-(trifluoromethyl)pyrazolo [1,5-a][1,3,5]triazin-4-amine

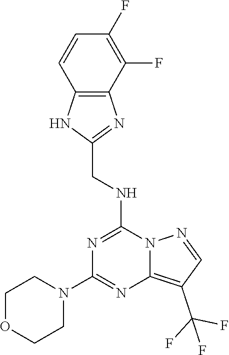 [SMILES: Fc1ccc2[nH]c(CNc3nc(N4CCOCC4)nc4c(C(F)(F)F)cnn34)nc2c1F]

To a solution of N-[(4,5-difluoro-1-{[2-(trimethylsilyl)ethoxy]methyl}-1H-benzimidazol-2-yl)methyl]-2-(morpholin-4-yl)-8-(trifluoromethyl)pyrazolo[1,5-a][1,3,5]triazin-4-amine (Intermediate 269, 150 mg, 78% purity, 0.2 mmol) in dichloromethane (5.0 mL) was added trifluoroacetic acid (5.0 mL) in one portion at room temperature. The reaction mixture was stirred at room temperature for 16 hours. The reaction mixture was concentrated and purified by preparative HPLC (Instrument: Gilson-281; Column: Phenomenex Synergi C18 150*25*10 μm; eluent A: water (0.225% formic acid), eluent B: acetonitrile; gradient: 0-10 min 18-48% B; flow 25 mL/min; temperature: room temperature; Detector: UV 220/254 nm) to give N-[(4,5-difluoro-1H-benzimidazol-2-yl)methyl]-2-(morpholin-4-yl)-8-(trifluoromethyl)pyrazolo[1,5-a][1,3,5]triazin-4-amine (53.4 mg, 96% purity, 56% yield) as an off-white solid.

LC-MS (Method C): Rₜ=0.90 min; MS (ESIpos): m/z=455.4 [M+H]⁺.

¹H NMR (400 MHz, DMSO-d₆): 5 [ppm]=12.7 (s, 1H), 9.33 (s, 1H), 8.26 (s, 1H), 7.23-7.18 (m, 2H), 4.90 (s, 2H), 3.80-3.50 (m, 4H), 3.50-3.23 (m, 4H).

### Example 427

### 2-(morpholin-4-yl)-N-[(5-phenyl-4H-1,2,4-triazol-3-yl)methyl]-8-(trifluoromethyl)pyrazolo[1,5-a][1,3,5]triazin-4-amine

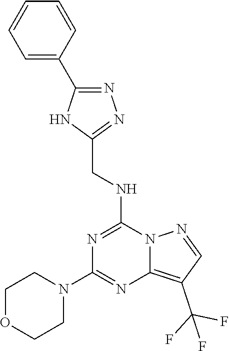 [SMILES: FC(F)(F)c1cnn2c(NCc3nnc(-c4ccccc4)[nH]3)nc(N3CCOCC3)nc12]

The reaction was performed in two separate batches. To a solution of 2-(morpholin-4-yl)-N-({3-phenyl-1-[(2RS)-tetrahydro-2H-pyran-2-yl]-1H-1,2,4-triazol-5-yl}methyl)-8-(trifluoromethyl)pyrazolo[1,5-a][1,3,5]triazin-4-amine (Intermediate 274, 30/270 mg, 80% purity, 45.3 μmol/0.41 mmol) in methanol (1.0/4.0 mL) was added p-toluenesulfonic acid (15.6/140 mg, 90.6 μmol/0.82 mmol) at room temperature. After stirring at room temperature for 16 hours, the reactions were combined, concentrated and purified by preperative HPLC [Instrument:ACSWH-GX-G; Column: Phenomenex Synergi C18 150*25*10 μm; eluent A: water (0.225% formic acid), eluent B: acetonitrile; gradient: 0-8.5 min 45-75% B; flow 25 mL/min; temperature: RT; Detector: UV 220/254 nm] to give 2-(morpholin-4-yl)-N-[(5-phenyl-4H-1,2,4-triazol-3-yl)methyl]-8-(trifluoromethyl)pyrazolo[1,5-a][1,3,5]triazin-4-amine (104 mg, 95% purity, 54% yield) as a gray solid.

LC-MS (Method C): Rₜ=0.92 min; MS (ESIpos): m/z=446.3 [M+H]⁺.

¹H NMR (400 MHz, DMSO-d₆) 5 [ppm]=14.43-13.61 (m, 1H), 9.45-9.04 (m, 1H), 8.24 (s, 1H), 8.06-7.83 (m, 2H), 7.60-7.29 (m, 3H), 4.92-4.59 (m, 2H), 3.77-3.63 (m, 4H), 3.61-3.41 (m, 4H).

### Example 428

### N-(1H-benzimidazol-2-ylmethyl)-8-(2,2-difluorocyclopropyl)-2-(4-methylpiperazin-1-yl)pyrazolo[1,5-a][1,3,5]triazin-4-amine

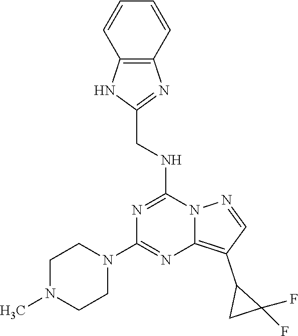 [SMILES: CN1CCN(c2nc(NCc3nc4ccccc4[nH]3)n3ncc(C4CC4(F)F)c3n2)CC1]

A solution of 8-(2,2-difluorocyclopropyl)-N-(4-methoxybenzyl)-2-(4-methylpiperazin-1-yl)-N-[(1-{[2-(trimethylsilyl)ethoxy]methyl}-1H-benzimidazol-2-yl)methyl]pyrazolo[1,5-a][1,3,5]triazin-4-amine (Intermediate 280, 50.0 mg, 0.73 mmol) in trifluoroacetic acid (2.0 mL, 26.0 mmol) was stirred at 80° C. for 16 hours. Saturated sodium bicarbonate solution was added to adjust pH-7 and concentrated to give a crude product. The crude product was purified by preparative HPLC [Instrument:GX-D; Column:Phenomenex Gemini NX-C18(75*30 mm*3 μm); eluent A: water (10 mM ammonia bicarbonate), eluent B: acetonitrile; gradient: 0-8 min 22-52% B; flow 30 mL/min; temperature: RT; Detector: UV 220/254 nm] to give N-(1H-benzimidazol-2-ylmethyl)-8-(2,2-difluorocyclopropyl)-2-(4-methylpiperazin-1-yl)pyrazolo[1,5-a][1,3,5]triazin-4-amine (4.50 mg, 14% yield) as a white solid.

LC-MS (Method C): Rₜ=0.57 min; MS (ESIpos): m/z=440.2 [M+H]⁺.

1H-NMR (400 MHz, DMSO-d₆)): 6 [ppm]=12.26 (s, 1H), 9.02-8.89 (m, 1H), 7.84 (s, 1H), 7.54 (d, J=5.2 Hz, 1H), 7.41 (d, J=6.0 Hz, 1H), 7.02 (d, J=5.2 Hz, 2H), 4.86 (d, J=5.2 Hz, 2H), 3.74-3.50 (m, 4H), 2.79-2.69 (m, 1H), 2.32-2.09 (m, 7H), 2.05-1.88 (m, 2H).

### Example 429

### N-[(4,5-difluoro-1H-benzimidazol-2-yl)methyl]-8-(2,2-difluorocyclopropyl)-2-(4-methylpiperazin-1-yl)pyrazolo[1,5-a][1,3,5]triazin-4-amine

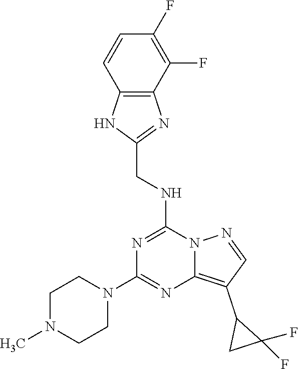 [SMILES: CN1CCN(c2nc(NCc3nc4c(F)c(F)ccc4[nH]3)n3ncc(C4CC4(F)F)c3n2)CC1]

The reaction was performed in two separate batches. To a solution of N-((4,5-difluoro-1H-benzo[d]imidazol-2-yl)methyl)-8-(2,2-difluorocyclopropyl)-2-(piperazin-1-yl)pyrazolo[1,5-a][1,3,5]triazin-4-amine (Intermediate 282, 10/70.0 mg, 69% purity, 21.7 μmol/0.11 mmol) in 1,2-dichloroethane (0.71/2.0 mL) were added formaldehyde (6 μL/0.03 mL, 37% purity, 86.7 μmol/0.42 mmol) and sodium triacetoxyborohydride (18.4/88.7 mg, 86.7 μmol/0.42 mmol) at 25° C. After stirring at 50° C. for 16 hours, the mixtures were combined, diluted with water, extracted with ethyl acetate. The organic phase was washed with brine, dried over anhydrous sodium sulfate and filtered. The filtrate was concentrated and purified by preparative HPLC [Instrument: GX-D; Column: Phenomenex Gemini NX-C18 (75*30 mm*3 μm); eluent A: water (10 mM ammonia bicarbonate), eluent B: acetonitrile; gradient: 0-8 min 28-58% B; flow 30 mL/min; temperature: RT; Detector: UV 220/254 nm] to give N-[(4,5-difluoro-1H-benzimidazol-2-yl)methyl]-8-(2,2-difluorocyclopropyl)-2-(4-methylpiperazin-1-yl)pyrazolo[1,5-a][1,3,5]triazin-4-amine (4.30 mg, 97% purity, 8% yield) as a white solid.

LC-MS (Method C): Rₜ=0.66 min; MS (ESIpos): m/z=476.2 [M+H]⁺.

¹H NMR (400 MHz, DMSO-d₆) δ [ppm]=8.95 (s, 1H), 7.82 (s, 1H), 7.24-7.13 (m, 2H), 4.84 (s, 2H), 3.59 (s, 4H), 2.76-2.70 (m, 1H), 2.16 (s, 4H), 2.11 (s, 3H), 2.01-1.89 (m, 2H).

### Example 430

### 8-(2,2-difluorocyclopropyl)-N-{[5-(2-methoxyphenyl)-4H-1,2,4-triazol-3-yl]methyl}-2-(4-methylpiperazin-1-yl)pyrazolo[1,5-a][1,3,5]triazin-4-amine

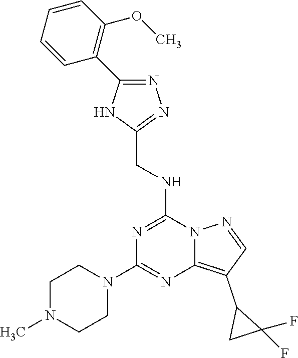 [SMILES: COc1ccccc1-c1nnc(CNc2nc(N3CCN(C)CC3)nc3c(C4CC4(F)F)cnn23)[nH]1]

To a solution of 8-(2,2-difluorocyclopropyl)-N-{[3-(2-methoxyphenyl)-1H-1,2,4-triazol-5-yl]methyl}-2-(piperazin-1-yl)pyrazolo[1,5-a][1,3,5]triazin-4-amine trifluoroacetate (1:1) (Intermediate 288, 100 mg, 60% purity, 0.1 mmol), paraformaldehyde (9.05 mg, 0.3 mmol) in 1,2-dichloroethane (3.0 mL) was added sodium triacetoxyborohydride (42.6 mg, 0.2 mmol) at room temperature. After stirring at 50° C. for 16 hours, the mixture was concentrated, dissolved in acetonitrile and purified by preperative HPLC [Instrument: ACSWH-GX-Q; Column:Shim-pack C18 150*25*10 μm; eluent A: water (0.225% formic acid), eluent B: acetonitrile; gradient: 0-10 min 18-38% B; flow 25 mL/min; temperature: RT; Detector: UV 220/254 nm] to give 8-(2,2-difluorocyclopropyl)-N-{[5-(2-methoxyphenyl)-4H-1,2,4-triazol-3-yl]methyl}-2-(4-methylpiperazin-1-yl)pyrazolo[1,5-a][1,3,5]triazin-4-amine (29.0 mg, 58% yield) as a white solid.

LC-MS (Method C): Rₜ=0.80 min; MS (ESIpos): m/z=497.3 [M+H]⁺.

¹H NMR (400 MHz, DMSO-d₆) 5 [ppm]=13.66-13.32 (m, 1H), 8.82 (t, J=5.2 Hz, 1H), 8.00 (d, J=6.8 Hz, 1H), 7.80 (s, 1H), 7.49-7.40 (m, 1H), 7.18 (d, J=8.4 Hz, 1H), 7.06 (t, J=7.6 Hz, 1H), 4.71 (d, J=5.6 Hz, 2H), 3.93 (s, 3H), 3.75-3.65 (m, 4H), 2.75-2.66 (m, 1H), 2.45-2.35 (m, 4H), 2.24 (s, 3H), 2.04-1.83 (m, 2H).

### Example 431

### N-[(1H-benzimidazol-2-yl)methyl]-8-ethyl-2-(morpholin-4-yl)pyrazolo[1,5-a][1,3,5]triazin-4-amine

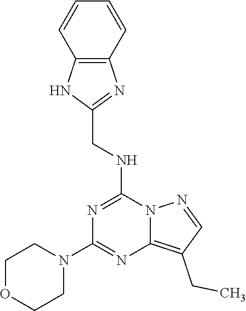 [SMILES: CCc1cnn2c(NCc3nc4ccccc4[nH]3)nc(N3CCOCC3)nc12]

A solution of 8-ethyl-N-[(4-methoxyphenyl)methyl]-2-(morpholin-4-yl)-N-[(1-{[2-(trimethylsilyl)ethoxy]methyl}-1H-benzimidazol-2-yl)methyl]pyrazolo[1,5-a][1,3,5]triazin-4-amine (Intermediate 291, 450 mg, 70% purity, 0.5 mmol) in trifluoroacetic acid (5.0 mL, 65 mmol) was stirred at 80° C. for 16 hours. The reaction mixture was concentrated to give a residue. The residue was poured into water. The mixture was adjusted to pH=7 with saturated sodium bicarbonate aqueous solution and then extracted with ethyl acetate. The organic phase was washed with brine, dried over anhydrous sodium sulfate and filtered. The filtrate was concentrated and purified by preparative HPLC [Instrument:ACSWH-GX-G; Column: Phenomenex Synergi C18 150*25*10 μm; eluent A: water (0.225% formic acid), eluent B: acetonitrile; gradient: 0-8.5 min 15-45% B; flow 25 mL/min; temperature: RT; Detector: UV 220/254 nm] to give N-(1H-benzimidazol-2-ylmethyl)-8-ethyl-2-(morpholin-4-yl)pyrazolo[1,5-a][1,3,5]triazin-4-amine (95.0 mg, 99% purity, 50% yield) as a white solid.

LC-MS (Method D): Rₜ=0.68 min; MS (ESIpos): m/z=379.1 [M+H]⁺.

¹H-NMR (400 MHz, DMSO-d₆): 5 [ppm]=12.74-12.52 (m, 1H), 8.87 (t, J=6.0 Hz, 1H), 8.14 (s, 1H), 7.82 (s, 1H), 7.50-7.48 (m, 2H), 7.18-7.13 (m, 2H), 4.89-4.87 (m, 2H), 3.59-3.57 (m, 4H), 3.50-3.48 (m, 4H), 2.49-2.47 (m, 2H), 1.19 (t, J=8.0 Hz, 3H).

### Example 432

### N-[(4,5-difluoro-1H-benzimidazol-2-yl)methyl]-8-ethyl-2-(morpholin-4-yl)pyrazolo[1,5-a][1,3,5]triazin-4-amine

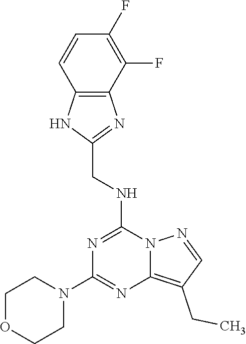 [SMILES: CCc1cnn2c(NCc3nc4c(F)c(F)ccc4[nH]3)nc(N3CCOCC3)nc12]

A solution of N-[(4,5-difluoro-1-{[2-(trimethylsilyl)ethoxy]methyl}-1H-benzimidazol-2-yl)methyl]-8-ethyl-N-(4-methoxybenzyl)-2-(morpholin-4-yl)pyrazolo[1,5-a][1,3,5]triazin-4-amine (Intermediate 292, 350 mg, 59% purity, 0.31 mmol) in trifluoroacetic acid (3.0 mL, 39 mmol) was stirred at 80° C. for 16 hours. The reaction mixture was concentrated to give a residue. The residue was poured into water. The mixture was adjusted to pH=7 with saturated sodium bicarbonate aqueous solution and then extracted with ethyl acetate. The organic phase was washed with brine, dried over anhydrous sodium sulfate and filtered. The filtrate was concentrated and purified by preparative HPLC [Instrument: ACSWH-GX-G; Column: Phenomenex Synergi C18 150*25 mm*10 μm; eluent A: water (0.225% formic acid), eluent B: acetonitrile; gradient: 0-8.5 min 38-68% B; flow 25 mL/min; temperature: RT; Detector: UV 220/254 nm] to give N-[(4,5-difluoro-1H-benzimidazol-2-yl)methyl]-8-ethyl-2-(morpholin-4-yl)pyrazolo[1,5-a][1,3,5]triazin-4-amine (29.4 mg, 99% purity, 23% yield) as a white solid.

LC-MS (Method C): Rₜ=0.75 min; MS (ESIpos): m/z=415.1 [M+H]⁺.

¹H-NMR (400 MHz, DMSO-d₆): 5 [ppm]=12.69 (s, 1H), 8.92 (s, 1H), 8.27 (s, 1H), 7.82 (s, 1H), 7.22-7.18 (m, 2H), 4.87 (d, J=5.2 Hz, 2H), 3.59-3.56 (m, 4H), 3.50-3.49 (m, 4H), 2.50-2.46 (m, 2H), 1.19 (t, J=7.6 Hz, 3H).

### Example 433

### 8-ethyl-N-[(4-methoxy-1H-benzimidazol-2-yl)methyl]-2-(morpholin-4-yl)pyrazolo[1,5-a][1,3,5]triazin-4-amine

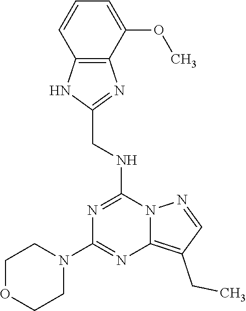 [SMILES: CCc1cnn2c(NCc3nc4c(OC)cccc4[nH]3)nc(N3CCOCC3)nc12]

A solution of 8-ethyl-N-(4-methoxybenzyl)-N-[(7-methoxy-1-{[2-(trimethylsilyl)ethoxy]methyl}-1H-benzimidazol-2-yl)methyl]-2-(morpholin-4-yl)pyrazolo[1,5-a][1,3,5]triazin-4-amine (Intermediate 293, 86.0 mg, 0.13 mmol) in trifluoroacetic acid (1.0 mL) was stirred at 80° C. for 16 hours. The reaction mixture was concentrated and purified by preparative HPLC [Instrument: ACSWH-GX-K; Column: Phenomenex Gemini-NX C18 75*30 mm*3 μm; eluent A: water (0.1% trifluoroacetic acid), eluent B: acetonitrile; gradient: 0-7 min 25-35% B; flow 25 mL/min; temperature: RT; Detector: UV 220/254 nm] to give 8-ethyl-N-[(4-methoxy-1H-benzimidazol-2-yl)methyl]-2-(morpholin-4-yl)pyrazolo[1,5-a][1,3,5]triazin-4-amine (14.7 mg, 99% purity, 27% yield) as a white solid.

LC-MS (Method K): Rₜ=9.04 min; MS (ESIpos): m/z=408.9 [M+H]⁺.

¹H-NMR (400 MHz, DMSO-d₆): 5 [ppm]=9.04 (t, J=5.6 Hz, 1H), 7.85 (s, 1H), 7.40 (t, J=8.0 Hz, 1H), 7.25 (d, J=8.0 Hz, 1H), 7.06 (d, J=8.0 Hz, 1H), 5.04 (d, J=4.0 Hz, 2H), 4.00 (s, 3H), 3.62-3.51 (m, 8H), 2.49-2.45 (m, 2H), 1.18 (t, J=7.6 Hz, 3H).

### Example 434

### N-[(1H-benzimidazol-2-yl)methyl]-8-ethyl-2-(4-methylpiperazin-1-yl)pyrazolo[1,5-a][1,3,5] triazin-4-amine

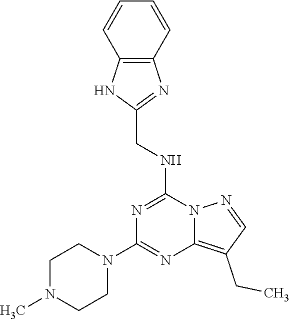 [SMILES: CCc1cnn2c(NCc3nc4ccccc4[nH]3)nc(N3CCN(C)CC3)nc12]

8-ethyl-N-[(4-methoxyphenyl)methyl]-2-(4-methylpiperazin-1-yl)-N-[(1-{[2-(trimethylsilyl) ethoxy]methyl}-1H-benzimidazol-2-yl)methyl]pyrazolo[1,5-a][1,3,5]triazin-4-amine (Intermediate 298, 40.0 mg, 74% purity, 0.046 mmol) was added to trifluoroacetic acid (2.0 mL, 26 mmol) at room temperature. The reaction was stirred at 80° C. for 16 hours. The solution was diluted with water, and adjusted to pH-7 by sodium bicarbonate and extracted with ethyl acetate. The organic phase was washed with brine, dried over anhydrous sodium sulfate and filtered. The filtrate was concentrated and purified by prepative HPLC [Instrument: ACSWH-GX-G; Column:Phenomenex Synergi C18 150*25*10 μm; eluent A: water (0.225% formic acid), eluent B: acetonitrile; gradient: 0-8.5 min 0-30% B; flow 25 mL/min; temperature: room temperature; Detector: UV 220/254 nm] to give N-(1H-benzimidazol-2-ylmethyl)-8-ethyl-2-(4-methylpiperazin-1-yl)pyrazolo[1,5-a][1,3,5]triazin-4-amine (4.10 mg, 98% purity, 22% yield) as a white solid.

LC-MS (Method C): Rₜ=0.650 min; MS (ESIpos): m/z=392.4 [M+H]⁺.

¹H NMR (400 MHz, DMSO-d₆) 5 [ppm]=12.38 (s, 1H), 9.00 (s, 1H), 7.85 (s, 1H), 7.48 (s, 2H), 7.20-7.10 (m, 2H), 4.88 (d, J=3.6 Hz, 2H), 3.35-3.21 (m, 4H), 3.10-2.70 (m, 4H), 2.62 (s, 3H), 2.46 (s, 2H), 1.19 (t, J=7.6 Hz 3H).

### Example 435

### formic acid N-[(4,5-difluoro-1H-benzimidazol-2-yl)methyl]-8-ethyl-2-(4-methylpiperazin-1-yl)pyrazolo[1,5-a][1,3,5]triazin-4-amine

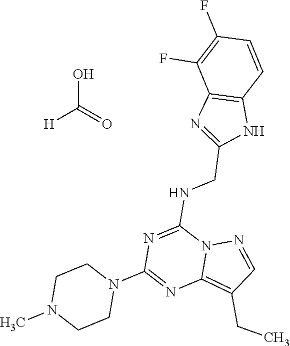 [SMILES: CCc1cnn2c(NCc3nc4c(F)c(F)ccc4[nH]3)nc(N3CCN(C)CC3)nc12 ; O=CO]

N-[(4,5-difluoro-1-{[2-(trimethylsilyl)ethoxy]methyl}-1H-benzimidazol-2-yl)methyl]-8-ethyl-N-(4-methoxybenzyl)-2-(4-methylpiperazin-1-yl)pyrazolo[1,5-a][1,3,5]triazin-4-amine (Intermediate 300, 220 mg, 51% purity, 0.165 mmol) was added to trifluoroacetic acid (5.0 mL) at room temperature. After stirring at 80° C. for 16 hours, the solution was diluted with water and adjusted to pH-7 by sodium bicarbonate, extracted with ethyl acetate. The organic phase was washed with brine, dried over anhydrous sodium sulfate and filtered. The filtrate was concentrated and purified by prepative HPLC [Instrument: ACSWH-GX-P; Column:Phenomenex Synergi C18 150*25 mm*10 μm; eluent A: water (0.225% formic acid), eluent B: acetonitrile; gradient: 0-11 min 5-35% B; flow 25 mL/min; temperature: room temperature; Detector: UV 220/254 nm] to give formic acid N-[(4,5-difluoro-1H-benzimidazol-2-yl)methyl]-8-ethyl-2-(4-methylpiperazin-1-yl)pyrazolo[1,5-a][1,3,5]triazin-4-amine (41.3 mg, 97% purity, 51.1% yield) as a light green solid.

LC-MS (Method C): Rₜ=0.758 min; MS (ESIpos): m/z=428.3 [M+H]⁺.

¹H NMR (400 MHz, DMSO-d₆) 5 [ppm]=12.67 (s, 1H), 8.92 (s, 1H), 7.82 (s, 1H), 7.27-7.14 (m, 2H), 4.86 (d, J=5.6 Hz, 2H), 3.62 (s, 4H), 2.49-2.45 (m, 2H), 2.36-2.26 (m, 4H), 2.22 (s, 3H), 1.19 (t, J=7.2 Hz, 3H).

### Example 436

### N-[(1H-benzimidazol-2-yl)methyl]-2-(4-methylpiperazin-1-yl)-8-(2,2,2-trifluoroethyl)pyrazolo [1,5-a][1,3,5]triazin-4-amine

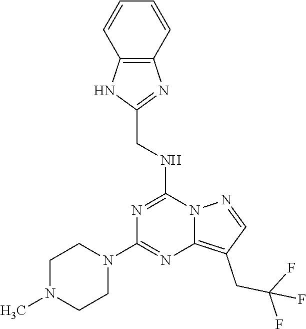 [SMILES: CN1CCN(c2nc(NCc3nc4ccccc4[nH]3)n3ncc(CC(F)(F)F)c3n2)CC1]

N-[(4-methoxyphenyl)methyl]-2-(4-methylpiperazin-1-yl)-8-(2,2,2-trifluoroethyl)-N-[(1-{[2-(trimethylsilyl)ethoxy]methyl}-1H-benzimidazol-2-yl)methyl]pyrazolo[1,5-a][1,3,5]triazin-4-amine (Intermediate 306, 60.0 mg, 72% purity, 0.06 mmol) was added to trifluoroacetic acid (1.0 mL, 13 mmol) at room temperature. After stirring at 80° C. for 16 hours, the solution was concentrated and diluted with water. The solution was adjusted to pH-7 by sodium bicarbonate and extracted with ethyl acetate. The organic phase was washed with brine, dried over anhydrous sodium sulfate and filtered. The filtrate was concentrated and purified by prepative HPLC [Instrument: GX-D; Column: Phenomenex Gemini NX-C18(75*30 mm*3 μm); eluent A: water (10 mM ammonium bicarbonate), eluent B: acetonitrile; gradient: 0-8 min 24-54% B; flow 30 mL/min; temperature: room temperature; Detector: UV 220/254 nm] to give N-(1H-benzimidazol-2-ylmethyl)-2-(4-methylpiperazin-1-yl)-8-(2,2,2-trifluoroethyl)pyrazolo[1,5-a][1,3,5]triazin-4-amine (4.80 mg, 17% yield) as a white solid.

LC-MS (Method C): Rₜ=0.683 min; MS (ESIpos): m/z=446.4 [M+H]⁺.

¹H NMR (400 MHz, DMSO-d₆) 5 [ppm]=9.39 (s, 1H), 8.01 (s, 1H), 7.78-7.64 (m, 2H), 7.53-7.46 (m, 2H), 5.22-5.11 (m, 2H), 4.56 (d, J=14.4 Hz, 2H), 3.51 (d, J=11.6 Hz, 4H), 3.25-3.09 (m, 2H), 2.93-2.78 (m, 2H), 2.73 (s, 3H).

### Example 437

### N-[(4,5-difluoro-1H-benzimidazol-2-yl)methyl]-2-(4-methylpiperazin-1-yl)-8-(2,2,2-trifluoroethyl) pyrazolo[1,5-a][1,3,5]triazin-4-amine

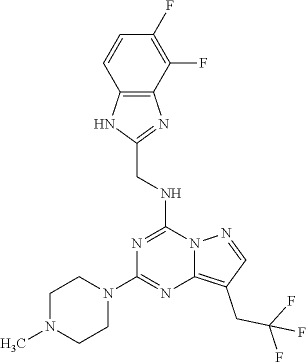 [SMILES: CN1CCN(c2nc(NCc3nc4c(F)c(F)ccc4[nH]3)n3ncc(CC(F)(F)F)c3n2)CC1]

N-[(4,5-difluoro-1-{[2-(trimethylsilyl)ethoxy]methyl}-1H-benzimidazol-2-yl)methyl]-N-[(4-methoxyphenyl)methyl]-2-(4-methylpiperazin-1-yl)-8-(2,2,2-trifluoroethyl)pyrazolo[1,5-a][1,3,5] triazin-4-amine (Intermediate 308, 60.0 mg, 94% purity, 0.08 mmol) was added to trifluoroacetic acid (1.0 mL, 13 mmol) at room temperature. After stirring at 80° C. for 16 hours, the solution was concentrated and diluted with water. The mixture was adjusted to pH-7 by sodium bicarbonate and extracted with ethyl acetate. The organic phase was washed with water and brine, dried over anhydrous sodium sulfate and filtered. The filtrate was concentrated and purified by prepative HPLC [Instrument: GX-D; Column: Phenomenex Gemini NX-C18(75*30 mm*3 μm); eluent A: water (10 mM ammonium bicarbonate), eluent B: acetonitrile; gradient: 0-8 min 26-56% B; flow 30 mL/min; temperature: room temperature; Detector: UV 220/254 nm] to give N-[(4,5-difluoro-1H-benzimidazol-2-yl)methyl]-2-(4-methylpiperazin-1-yl)-8-(2,2,2-trifluoroethyl)pyrazolo[1,5-a][1,3,5]triazin-4-amine (3.80 mg, 10% yield) as a white solid.

LC-MS (Method C): Rₜ=0.781 min; MS (ESIpos): m/z=482.4 [M+H]⁺.

¹H NMR (400 MHz, DMSO-d₆) 5 [ppm]=9.27 (t, J=5.6 Hz, 1H), 7.98 (s, 1H), 7.35-7.18 (m, 2H), 4.92 (s, 2H), 4.61 (d, J=14.8 Hz, 2H), 3.61-3.28 (m, 4H), 3.17 (d, J=10.8 Hz, 2H), 2.90 (d, J=8.8 Hz, 2H), 2.74 (s, 3H).

### Example 438

### trifluoroacetic acid N-[(1H-benzimidazol-2-yl)methyl]-2-(morpholin-4-yl)-8-(pyridazin-4-yl)pyrazolo[1,5-a][1,3,5]triazin-4-amine

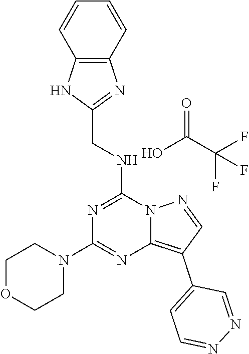

A solution of N-[(4-methoxyphenyl)methyl]-2-(morpholin-4-yl)-8-(pyridazin-4-yl)-N-[(1-{[2-(trimethylsilyl)ethoxy]methyl}-1H-benzimidazol-2-yl)methyl]pyrazolo[1,5-a][1,3,5]triazin-4-amine (Intermediate 309, 200 mg, 67% purity, 0.197 mmol) in trifluoroacetic acid (4.0 mL) was stirred at 80° C. for 4 hours. The solution was concentrated, diluted with methanol, and adjusted to pH=7 by sodium bicarbonate. The mixture was filtered and the filtrate was concentrated, purified by preparative HPLC [Instrument: GX-U; Column: Phenomenex luna C18 150*40 mm*15 μm; eluent A: water (0.1% trifluoroacetic acid), eluent B: acetonitrile; gradient: 0-60 min 3-33% B; flow 60 mL/min; temperature: room temperature; Detector: UV 220/254 nm] to give N-(4-methoxybenzyl)-2-(morpholin-4-yl)-8-(pyridazin-4-yl)-N-[(1-{[2-(trimethylsilyl)ethoxy]methyl}-1H-benzimidazol-2-yl)methyl]pyrazolo[1,5-a][1,3,5]triazin-4-amine (105 mg, 98% purity, 96% yield) as a yellow solid.

LC-MS (Method C): Rₜ=0.68 min; MS (ESIpos): m/z=428.9 [M+H]⁺.

¹H NMR (400 MHz, DMSO-d₆) 5 [ppm]=9.87 (d, J=1.2 Hz, 1H), 9.55 (t, J=5.2 Hz, 1H), 9.17 (d, J=6.0 Hz, 1H), 8.90 (s, 1H), 8.43 (dd, J=5.2, 1.6 Hz, 1H), 7.73 (q, J=3.2 Hz, 2H), 7.47 (q, J=3.2, Hz, 2H), 5.15 (d, J=5.2 Hz, 2H), 3.88-3.78 (m, 4H), 3.65-3.55 (m, 4H).

### Example 439

### trifluoroacetic acid N-[(7-methoxy-1H-benzimidazol-2-yl)methyl]-2-(morpholin-4-yl)-8-(pyridazin-4-yl)pyrazolo[1,5-a][1,3,5]triazin-4-amine

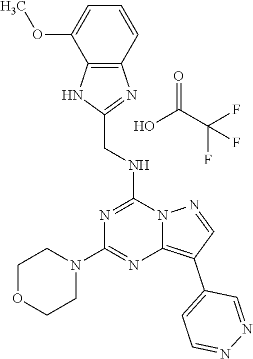

A solution of N-[(4-methoxyphenyl)methyl]-N-[(7-methoxy-1-{[2-(trimethylsilyl)ethoxy]methyl}-1H-benzimidazol-2-yl)methyl]-2-(morpholin-4-yl)-8-(pyridazin-4-yl)pyrazolo[1,5-a][1,3,5]triazin-4-amine (Intermediate 310, 310 mg, 0.437 mmol) in trifluoroacetic acid (2.0 mL, 26 mmol) was stirred at 80° C. for 6 hours. The reaction mixture was concentrated and purified by preparative HPLC [Instrument:GX-U; Column: Phenomenex luna C18 150*40 mm*15 μm; eluent A: water (0.225% trifluoroacetic acid), eluent B: acetonitrile; gradient: 0-10 min 4-34% B; flow 25 mL/min; temperature: RT; Detector: UV 220/254 nm] to give N-[(7-methoxy-1H-benzimidazol-2-yl)methyl]-2-(morpholin-4-yl)-8-(pyridazin-4-yl)pyrazolo[1,5-a][1,3,5]triazin-4-amine trifluoroacetate (104 mg, 99% purity, 41% yield) as a yellow solid.

LC-MS (Method D): Rₜ=0.604 min; MS (ESIpos): m/z=459.1 [M+H]⁺.

¹H-NMR (400 MHz, DMSO-d₆)): 5 [ppm]=9.88 (d, J=1.6 Hz, 1H), 9.50 (t, J=5.6 Hz, 1H), 9.19 (dd, J=6.0, 0.8 Hz, 1H), 8.89 (s, 1H), 8.47 (dd, J=6.0, 2.4 Hz, 1H), 7.45-7.36 (m, 1H), 7.28 (d, J=8.0 Hz, 1H), 7.05 (d, J=8.0 Hz, 1H), 5.12 (d, J=5.6 Hz, 2H), 3.99 (s, 3H), 3.91-3.27 (m, 8H).

### Example 440

### trifluoroacetic acid N-[(5,6-dichloro-1H-benzimidazol-2-yl)methyl]-2-(morpholin-4-yl)-8-(pyridazin-4-yl)pyrazolo[1,5-a][1,3,5]triazin-4-amine

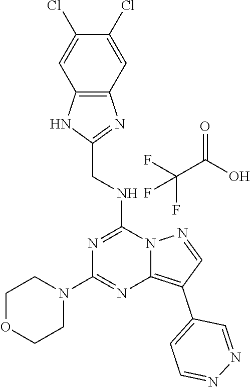

A solution of N-[(5,6-dichloro-1-{[2-(trimethylsilyl)ethoxy]methyl}-1H-benzimidazol-2-yl)methyl]-N-[(4-methoxyphenyl)methyl]-2-(morpholin-4-yl)-8-(pyridazin-4-yl)pyrazolo[1,5-a][1,3,5]triazin-4-amine (Intermediate 311, 128 mg, 0.171 mmol) in trifluoroacetic acid (2.0 mL) was stirred at 80° C. for 2 hours. The reaction mixture was concentrated and purified by preparative HPLC [Instrument: ACSWH-GX-N; Column: Phenomenex Synergi C18 150*25 mm*4 μm; eluent A: water (0.225% trifluoroacetic acid), eluent B: acetonitrile; gradient: 0-40 min 20-50% B; flow 25 mL/min; temperature: RT; Detector: UV 220/254 nm] to give N-[(5,6-dichloro-1H-benzimidazol-2-yl)methyl]-2-(morpholin-4-yl)-8-(pyridazin-4-yl)pyrazolo[1,5-a][1,3,5]triazin-4-amine trifluoroacetate (48.7 mg, 99% purity, 46% yield) as a yellow solid.

LC-MS (Method D): Rₜ=0.695 min; MS (ESIpos): m/z=497.1 [M+H]⁺.

¹H-NMR (400 MHz, DMSO-d₆)): 5 [ppm]=9.88 (d, J=1.6 Hz, 1H), 9.46 (t, J=5.2 Hz, 1H), 9.20 (d, J=6.0 Hz, 1H), 8.90 (s, 1H), 8.50 (s, 1H), 7.79 (s, 2H), 4.93 (d, J=5.6 Hz, 2H), 3.78-3.71 (m, 8H).

### Example 441

### trifluoroacetic acid-N-[(1H-benzimidazol-2-yl)methyl]-2-(morpholin-4-yl)-8-(1,3-thiazol-5-yl)pyrazolo[1,5-a][1,3,5]triazin-4-amine (1/1)

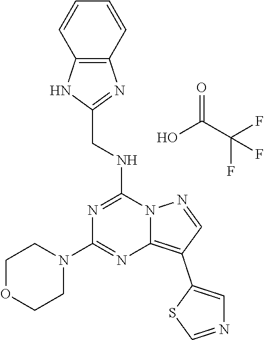 [SMILES: c1ccc2[nH]c(CNc3nc(N4CCOCC4)nc4c(-c5cncs5)cnn34)nc2c1 ; O=C(O)C(F)(F)F]

A solution of N-[(4-methoxyphenyl)methyl]-2-(morpholin-4-yl)-8-(1,3-thiazol-5-yl)-N-[(1-{[2-(trimethylsilyl)ethoxy]methyl}-1H-benzimidazol-2-yl)methyl]pyrazolo[1,5-a][1,3,5]triazin-4-amine (Intermediate 312, 230 mg, 91% purity, 0.31 mmol) in trifluoroacetic acid (4.0 mL) was stirred at 80° C. for 2 hours. The solution was concentrated, diluted with methanol. After adjusting to pH=7 by sodium bicarbonate powder, the mixture was filtered. The filtrate was concentrated and purified by preparative HPLC [Instrument: ACSWH-GX-N; Column: Phenomenex Synergi C18 150*25 mm*10 μm; eluent A: water (0.1% trifluoroacetic acid), eluent B: acetonitrile; gradient: 0-10 min 12-42% B; flow 25 mL/min; temperature: room temperature; Detector: UV 220/254 nm] to N-(1H-benzimidazol-2-ylmethyl)-2-(morpholin-4-yl)-8-(1,3-thiazol-5-yl)pyrazolo[1,5-a][1,3,5]triazin-4-amine trifluoroacetate (151 mg, 97% purity, 88% yield) as a brown solid.

LC-MS (Method C): Rₜ=0.76 min; MS (ESIpos): m/z=433.9 [M+H]⁺.

¹H NMR (400 MHz, DMSO-d₆) 5 [ppm]=9.42 (t, J=5.2 Hz, 1H), 8.91 (s, 1H), 8.47 (s, 1H), 8.24 (s, 1H), 7.80-7.72 (m, 2H), 7.56-7.48 (m, 2H), 5.16 (d, J=3.2 Hz, 2H), 3.75-3.55 (m, 4H), 3.54-3.34 (m, 4H).

### Example 442

### trifluoroacetic acid N-[(4,5-difluoro-1H-benzimidazol-2-yl)methyl]-2-(morpholin-4-yl)-8-(1,3-thiazol-5-yl)pyrazolo[1,5-a][1,3,5]triazin-4-amine

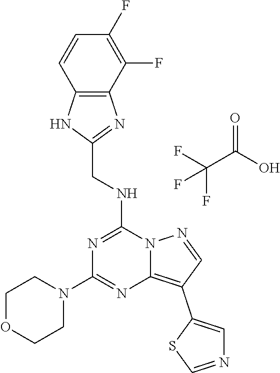

A solution of N-[(4,5-difluoro-1-{[2-(trimethylsilyl)ethoxy]methyl}-1H-benzimidazol-2-yl)methyl]-N-[(4-methoxyphenyl)methyl]-2-(morpholin-4-yl)-8-(1,3-thiazol-5-yl)pyrazolo[1,5-a][1,3,5]triazin-4-amine (Intermediate 313, 300 mg, 85% purity, 0.35 mmol) in trifluoroacetic acid (5 mL) was stirred at 80° C. for 2 hours. The solution was concentrated, diluted with methanol. After adjusting to pH=7 by sodium bicarbonate powder, the mixture was filtered. The filtrate was concentrated and purified by preparative HPLC [Instrument: GX-U; Column: Phenomenex luna C18 150*40 mm*15 μm; eluent A: water (0.1% trifluoroacetic acid), eluent B: acetonitrile; gradient: 0-10 min 23-53% B; flow 60 mL/min; temperature: room temperature; Detector: UV 220/254 nm] to N-[(4,5-difluoro-1H-benzimidazol-2-yl)methyl]-2-(morpholin-4-yl)-8-(1,3-thiazol-5-yl)pyrazolo[1,5-a][1,3,5]triazin-4-amine (92.8 mg, 99% purity, 44% yield) as a brown solid.

LC-MS (Method C): Rₜ=0.84 min; MS (ESIpos): m/z=469.9 [M+H]⁺.

¹H NMR (400 MHz, DMSO-d₆) 5 [ppm]=9.25 (t, J=6.0 Hz, 1H), 8.92 (s, 1H), 8.43 (s, 1H), 8.24 (s, 1H), 7.30-7.19 (m, 2H), 4.92 (d, J=5.6 Hz, 2H), 3.68 (s, 4H), 3.54 (s, 4H).

### Example 443

### N-[(5,6-dichloro-1H-benzimidazol-2-yl)methyl]-2-(morpholin-4-yl)-8-(1,3-thiazol-5-yl)pyrazolo[1,5-a][1,3,5]triazin-4-amine

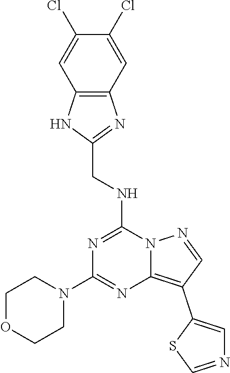 [SMILES: Clc1cc2nc(CNc3nc(N4CCOCC4)nc4c(-c5cncs5)cnn34)[nH]c2cc1Cl]

A solution of N-[(5,6-dichloro-1-{[2-(trimethylsilyl)ethoxy]methyl}-1H-benzimidazol-2-yl)methyl]-N-[(4-methoxyphenyl)methyl]-2-(morpholin-4-yl)-8-(1,3-thiazol-5-yl)pyrazolo[1,5-a][1,3,5]triazin-4-amine (Intermediate 314, 190 mg, 81% purity, 0.20 mmol) in trifluoroacetic acid (5.0 mL, 65 mmol) was stirred at 80° C. for 2 hours. The reaction mixture was concentrated to give a residue. The residue was purified by preparative HPLC [lnstrument:ACSWH-GX-N; Column: Phenomenex Synergi C18 150*25 mm*10 μm; eluent A: water (0.225% formic acid in water), eluent B: acetonitrile; gradient: 0-10 min 32-62% B; flow 25 mL/min; temperature: RT; Detector: UV 220/254 nm] to give N-[(5,6-dichloro-1H-benzimidazol-2-yl)methyl]-2-(morpholin-4-yl)-8-(1,3-thiazol-5-yl)pyrazolo[1,5-a][1,3,5]triazin-4-amine (46.9 mg, 96% purity, 44% yield) as a white solid.

LC-MS (Method C): Rₜ=0.88 min; MS (ESIpos): m/z=501.9 [M+H]⁺.

¹H NMR (400 MHz, DMSO-d₆) 5 [ppm]=9.24 (t, J=5.6 Hz, 1H), 8.91 (s, 1H), 8.43 (s, 1H), 8.23 (d, J=0.6 Hz, 1H), 7.80 (s, 2H), 4.93 (d, J=5.6 Hz, 2H), 3.56-3.46 (m, 8H).

### Example 444

### trifluoroacetic acid N-[(1H-benzimidazol-2-yl)methyl]-8-(2,2-difluoroethyl)-2-(morpholin-4-yl)pyrazolo[1,5-a][1,3,5]triazin-4-amine

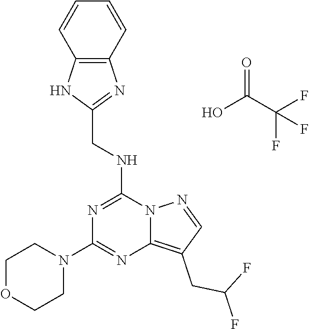 [SMILES: FC(F)Cc1cnn2c(NCc3nc4ccccc4[nH]3)nc(N3CCOCC3)nc12 ; O=C(O)C(F)(F)F]

A solution of 8-(2,2-difluoroethyl)-N-[(4-methoxyphenyl)methyl]-2-(morpholin-4-yl)-N-[(1-{[2-(trimethylsilyl)ethoxy]methyl}-1H-benzimidazol-2-yl)methyl]pyrazolo[1,5-a][1,3,5]triazin-4-amine (Intermediate 315, 180 mg, 99% purity, 0.27 mmol) in trifluoroacetic acid (4.0 mL, 52 mmol) was stirred at 80° C. for 16 hours. The solution was concentrated, diluted with methanol. After adjusting to pH=7 by sodium bicarbonate powder, the mixture was filtered. The filtrate was concentrated and purified by preparative HPLC [Instrument: ACSWH-GX-N; Column: Phenomenex Synergi C18 150*25 mm*10 μm; eluent A: water (0.1% trifluoroacetic acid), eluent B: acetonitrile; gradient: 0-10 min 20-50% B; flow 25 mL/min; temperature: room temperature; Detector: UV 220/254 nm] to give N-(1H-benzimidazol-2-ylmethyl)-8-(2,2-difluoroethyl)-2-(morpholin-4-yl)pyrazolo[1,5-a][1,3,5]triazin-4-amine trifluoroacetate (1:1) (35.7 mg, 99% purity, 25% yield) as a white solid.

LC-MS (Method C): Rₜ=0.76 min; MS (ESIpos): m/z=414.9 [M+H]⁺.

¹H NMR (400 MHz, DMSO-d₆) 5 [ppm]=9.26 (t, J=5.2 Hz, 1H), 7.91 (s, 1H), 7.81-7.69 (m, 2H), 7.59-7.47 (m, 2H), 6.22 (tt, J=56.8, 4.0 Hz, 1H), 5.14 (d, J=5.2 Hz, 2H), 3.51 (s, 4H), 3.42 (s, 4H), 3.06 (td, J=18.0, 4.0 Hz, 2H).

### Experimental Section—Biological Assays

Examples were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batch.

An empty field in any of the following tables means that the respective compound has not been tested in that Assay.

### 1. Expression and Purification of the CDK12/CycK and CDK13/CycK Used in the CDK12 and CDK13 Kinase Activity Assays

### 1.1 Cloning of CDK 12/13, CycK and CAK1 in Insect Destination Vectors

The cDNAs encoding the following protein sequences were codon optimized for expression in Sf9/Hi-5 insect cells and synthesized by the GeneArt Technology at Thermo Fischer Scientific.

The human CDK12 wt/DN (Acc. Q9NYV4), CDK13 (Q14004), CycK (075909) and the Saccharomyces cerevisiae CAK1 (P43568) full length sequence were used for cloning.

These cDNAs also encoded att-site sequences at the 5′ and 3′ ends for subcloning into the following destination vectors using the Gateway Technology.

By using of baculovirus vectors with a strong polyhedrin promoter provides an N-terminal fusion of a His-tag with a Tobacco Edge virus cleavage site to the integrated gene of interest. Only the Saccharomyces cerevisiae CAK1 (P43568) full length sequence was cloned in an insect vector which provides a tag-free gene of interest.

### 1.2 Sequence

His-CDK12 (aa Q696-S1082)
MTSHHHHHHS SMGSRTSLYK KAGSDYDIPT TENLYFQGQP YKKRPKICCP
RYGERRQTES DWGKRCVDKF DIIGIIGEGT YGQVYKAKDK DTGELVALKK
VRLDNEKEGF PITAIREIKI LRQLIHRSVV NMKEIVTDKQ DALDFKKDKG
AFYLVFEYMD HDLMGLLESG LVHFSEDHIK SFMKQLMEGL EYCHKKNFLH
RDIKCSNILL NNSGQIKLAD FGLARLYNSE ESRPYTNKVI TLWYRPPELL
LGEERYTPAI DVWSCGCILG ELFTKKPIFQ ANLELAQLEL ISRLCGSPCP
AVWPDVIKLP YFNTMKPKKQ YRRRLREEFS FIPSAALDLL DHMLTLDPSK
RCTAEQTLQS DFLKDVELSK MAPPDLPHWQ DCHELWSKKR RRQRQSGVVV
EEPPPSKTSR KETTSGTSTE PVKNS
His-CDK12-DN (aa Q696-S1082; K756A; D877N)
MTSHHHHHHS SMGSRTSLYK KAGSDYDIPT TENLYFQGQP YKKRPKICCP
RYGERRQTES DWGKRCVDKF DIIGIIGEGT YGQVYKAKDK DTGELVALAK
VRLDNEKEGF PITAIREIKI LRQLIHRSVV NMKEIVTDKQ DALDFKKDKG
AFYLVFEYMD HDLMGLLESG LVHFSEDHIK SFMKQLMEGL EYCHKKNFLH
RDIKCSNILL NNSGQIKLAN FGLARLYNSE ESRPYTNKVI TLWYRPPELL
LGEERYTPAI DVWSCGCILG ELFTKKPIFQ ANLELAQLEL ISRLCGSPCP
AVWPDVIKLP YFNTMKPKKQ YRRRLREEFS FIPSAALDLL DHMLTLDPSK
RCTAEQTLQS DFLKDVELSK MAPPDLPHWQ DCHELWSKKR RRQRQSGVVV
EEPPPSKTSR KETTSGTSTE PVKNS
His-CDK13 (aa Q673-P1059)
MTSHHHHHHS SMGSRTSLYK KAGSDYDIPT TENLYFQGQL HSKRRPKICG
PRYGETKEKD IDWGKRCVDK FDIIGIIGEG TYGQVYKARD KDTGEMVALK
KVRLDNEKEG FPITAIREIK ILRQLTHQSI INMKEIVTDK EDALDFKKDK GAFYLVFEYM
DHDLMGLLES GLVHFNENHI KSFMRQLMEG LDYCHKKNFL HRDIKCSNIL
LNNRGQIKLA DFGLARLYSS EESRPYTNKV ITLWYRPPEL LLGEERYTPA
IDVWSCGCIL GELFTKKPIF QANQELAQLE LISRICGSPC PAVWPDVIKL
PYFNTMKPKK QYRRKLREEF VFIPAAALDL FDYMLALDPS KRCTAEQALQ
CEFLRDVEPS KMPPPDLPLW QDCHELWSKK RRRQKQMGMT DDVSTIKAPR
KDLSLGLDDS RTNTP
His-CDK13-DN (aa Q673-P1059; K734A; D855N)
MTSHHHHHHS SMGSRTSLYK KAGSDYDIPT TENLYFQGQL HSKRRPKICG
PRYGETKEKD IDWGKRCVDK FDIIGIIGEG TYGQVYKARD KDTGEMVALA
KVRLDNEKEG FPITAIREIK ILRQLTHQSI INMKEIVTDK EDALDFKKDK GAFYLVFEYM
DHDLMGLLES GLVHFNENHI KSFMRQLMEG LDYCHKKNFL HRDIKCSNIL
LNNRGQIKLA NFGLARLYSS EESRPYTNKV ITLWYRPPEL LLGEERYTPA
IDVWSCGCIL GELFTKKPIF QANQELAQLE LISRICGSPC PAVWPDVIKL
PYFNTMKPKK QYRRKLREEF VFIPAAALDL FDYMLALDPS KRCTAEQALQ
CEFLRDVEPS KMPPPDLPLW QDCHELWSKK RRRQKQMGMT DDVSTIKAPR
KDLSLGLDDS RTNTP
His-CycK (aa M1-S300)
MTSHHHHHHS SMGSRTSLYK KAGSDYDIPT TENLYFQGMK ENKENSSPSV
TSANLDHTKP CWYWDKKDLA HTPSQLEGLD PATEARYRRE GARFIFDVGT
RLGLHYDTLA TGIIYFHRFY MFHSFKQFPR YVTGACCLFL AGKVEETPKK
CKDIIKTARS LLNDVQFGQF GDDPKEEVMV LERILLQTIK FDLQVEHPYQ
FLLKYAKQLK GDKNKIQKLV QMAWTFVNDS LCTTLSLQWE PEHAVAVMY
LAGRLCKFEI QEWTSKPMYR RWWEQFVQDV PVDVLEDICH QILDLYSQGK
QQMPHHTPHQ LQQPPSLQPT PQVPQVQQSQ PSQSSEPS
CAK1 (aa M1-P368)
MKLDSIDITH CQLVKSTRTA RIYRSDTYAI KCLALDFDIP PHNAKFEVSI
LNKLGNKCKH ILPLLESKAT DNNDLLLLFP FEEMNLYEFM QMHYKRDRRK
KNPYYDLLNP SIPIVADPPV QKYTNQLDVN RYSLSFFRQM VEGIAFLHEN
KIIHRDIKPQ NIMLTNNTST VSPKLYIIDF GISYDMANNS QTSAEPMDSK VTDISTGIYK
APEVLFGVKC YDGGVDVWSL LIIISQWFQR ETSRMGHVPA MIDDGSDDMN
SDGSDFRLIC SIFEKLGIPS IQKWEEVAQH GSVDAFVGMF GADGDGKYVL
DQEKDVQISI VERNMPRLDE IADVKVKQKF INCILGMVSF SPNERWSCQR ILQELEKP

### 1.3 Expression of the CDK12-CycK and CDK13-CycK Complex

The Hi-5 insect cells were cultivated in Insect Xpress Medium (Lonza #BE12-730Q) and for co-infection the following baculovirus with multiplicity of infection (MOI) was using for the expression of the complex: CDK12 and CDK13 with MOI 1.0; CycK and CAK1 with MOI 0.5.

The complex formation was performed by co-infection of Hi-5 cells grown in suspension to a density of 2×10⁶ cells/mL in 8 L waver for 72 h. The cells were harvested by centrifugation (10 min., 170 g, 4° C.) and the cell pellets stored at −80° C. 1.4 Purification of the CDK12 and CDK13 complex

Purification of the His-CDK12/His-CycK/CAK1 or His-CDK13/His-CycK/CAK1 complex was achieved by affinity chromatography using Ni-Sepharose High Performance (GE Healthcare #17-5268-02) or HisTrap™HP (GE Healthcare #17-5247-01/05)

Cell pellets were resuspended in lysis buffer (50 millimol/L Hepes pH 7.5, 500 millimol/L NaCl, 40 millimol/L Imidazol, 10% Glycerol; 0.5% NP40, Benzonase (150 U/10 g cell pellet), 1 millimol/L DTT and 1×Complete EDTA-free protease inhibitor cocktail (Roche #1873580)).

The lysate was incubated on ice for 30 minutes and clarified by centrifugation (1 h, 4° C., 27500×g). Proteins were captured overnight at 4° C. using Ni-Sepharose or HisTrap HP material, washed with CDK12/13 wash buffer (50 millimol/L Hepes pH 7.5, 500 millimol/L NaCl, 40 millimol/L imidazole, 10% Glycerol, 1 millimol/L DTT) and eluted with wash buffer by using gradient of imidazole (40-500 millimol/L).

For removal of imidazole the eluted protein complexes were desalted with Zeba™ Desalt Spin Columns (Pierce #89893) against CDK12/13 DS buffer (50 millimol/L Hepes pH 7.5, 500 millimol/L NaCl, 10% Glycerol, 1 millimol/L DTT).

The final concentration was calculated densitrometrically using BSA as a standard in a Coomassie stained gel. Elution fractions were aliquoted and shock frozen using liquid nitrogen.

The in vitro activity of the compounds of the present invention can be demonstrated in the following assays:

### 2. Biochemical Kinase Assays

### 2.1 CDK12/CycK Low ATP Kinase Assay

CDK12/CycK-inhibitory activity of compounds of the present invention at 10 micromol/L adenosine-tri-phosphate (ATP) was quantified employing the TR-FRET (TR-FRET=T̅ime R̅esolved F̅luorescence Ēnergy T̅ransfer) based CDK12/CycK activity inhibition assay as described in the following paragraphs.

A complex of human recombinant CDK12 and human recombinant CycK (both N-terminally His-tagged, expression and purification as described above) was used as enzyme. As substrate for the kinase reaction biotinylated peptide biotin-Ahx-KFELLPTPPLSPSRRSGL (C-terminus in amid form) was used which can be purchased e.g. form the company Biosyntan (Berlin-Buch, Germany).

For the assay 50 nanoL of a 100 fold concentrated solution of the test compound in DMSO was pipetted into either a black low volume 384 well microtiter plate or a black 1536 well microtiter plate (both Greiner Bio-One, Frickenhausen, Germany), 2 microL of a solution of CDK12/CycK in aqueous assay buffer [25 millimol/L HEPES pH 7.5, 20 millimol/L MgCl₂, 5 millimol/L β-glycerophosphate, 2 millimol/L EGTA, 1.0 millimol/L dithiothreitol, 0.01% (v/v) Nonidet-P40 (Sigma), 0.01% (w/v) bovine serum albumin] were added and the mixture was incubated for 15 min at 22° C. to allow pre-binding of the test compounds to the enzyme before the start of the kinase reaction. Then the kinase reaction was started by the addition of 3 microL of a solution ATP (16.7 micromol/L=>final conc. in the 5 microL assay volume is 10 micromol/L) and substrate (1.67 micromol/L=>final conc. in the 5 microL assay volume is 1 micromol/L) in assay buffer and the resulting mixture was incubated for a reaction time of 60 min at 22° C. The concentration of CDK12/CycK was adjusted depending of the activity of the enzyme lot and was chosen appropriate to have the assay in the linear range, typical concentrations were about 2 nanomol/L. The reaction was stopped by the addition of 3 microL of a solution of TR-FRET detection reagents (125 nanomol/L streptavidine-XL665 [Cisbio Bioassays, Codolet, France] and 0.67 nanomol/L anti-Phospho-c-Myc (Ser 62) (E1J4K)-antibody from Cell Signalling [#13748] and 2 nanomol/L LANCE EU-W1024 labeled anti-rabbit IgG antibody [Perkin-Elmer, product no. 0083]) in an aqueous EDTA-solution (133 millimol/L EDTA, 0.27% (w/v) bovine serum albumin in 66.7 millimol/L HEPES pH 7.5).

The resulting mixture was incubated 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm was measured in a TR-FRET reader, e.g. a Pherastar FS (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 micromol/L to 0.07 nanomol/L (20 micromol/L, 5.7 micromol/L, 1.6 micromol/L, 0.47 micromol/L, 0.13 micromol/L, 38 nanomol/L, 11 nanomol/L, 3.1 nanomol/L, 0.9 nanomol/L, 0.25 nanomol/L and 0.07 nanomol/L, the dilution series prepared separately before the assay on the level of the 100 fold concentrated solutions in DMSO by serial dilutions, exact concentrations may vary depending pipettors used) in duplicate values for each concentration and IC₅₀ values were calculated using Genedata Screener™ software.

### 2.2 CDK12/CycK High ATP Kinase Assay

In the context of the present invention, the term “IC50 CDK12 hATP” refers to the IC₅₀ values obtained according to the assay described in this section (2.2) herein below, i.e. the IC₅₀ values for the inhibition of CDK12 at high (2 mM) ATP.

CDK12/CycK-inhibitory activity of compounds of the present invention at 2 millimol/L adenosine-tri-phosphate (ATP) was quantified employing the TR-FRET (TR-FRET=Time Resolved Fluorescence Energy Transfer) based CDK12/CycK activity inhibition assay as described in the following paragraphs.

A complex of human recombinant CDK12 and human recombinant CycK (both N-terminally His-tagged, expression and purification as described above) was used as enzyme. As substrate for the kinase reaction biotinylated peptide biotin-Ahx-KFELLPTPPLSPSRRSGL (C-terminus in amid form) was used which can be purchased e.g. form the company Biosyntan (Berlin-Buch, Germany).

For the assay 50 nanoL of a 100 fold concentrated solution of the test compound in DMSO was pipetted into either a black low volume 384 well microtiter plate or a black 1536 well microtiter plate (both Greiner Bio-One, Frickenhausen, Germany), 2 microL of a solution of CDK12/CycK in aqueous assay buffer [25 millimol/L HEPES pH 7.5, 20 millimol/L MgCl₂, 5 millimol/L β-glycerophosphate, 2 millimol/L EGTA, 1.0 millimol/L dithiothreitol, 0.01% (v/v) Nonidet-P40 (Sigma), 0.01% (w/v) bovine serum albumin] were added and the mixture was incubated for 15 min at 22° C. to allow pre-binding of the test compounds to the enzyme before the start of the kinase reaction. Then the kinase reaction was started by the addition of 3 microL of a solution ATP (3.33 millimol/L=>final conc. in the 5 microL assay volume is 2 millimol/L) and substrate (1.67 micromol/L=>final conc. in the 5 microL assay volume is 1 micromol/L) in assay buffer and the resulting mixture was incubated for a reaction time of 60 min at 22° C. The concentration of CDK12/CycK was adjusted depending of the activity of the enzyme lot and was chosen appropriate to have the assay in the linear range, typical concentrations were about 0.75 nanomol/L. The reaction was stopped by the addition of 3 microL of a solution of TR-FRET detection reagents (125 nanomol/L streptavidine-XL665 [Cisbio Bioassays, Codolet, France] and 0.67 nanomol/L anti-Phospho-c-Myc (Ser 62) (E1J4K)-antibody from Cell Signalling [#13748] and 2 nanomol/L LANCE EU-W1024 labeled anti-rabbit IgG antibody [Perkin-Elmer, product no. 0083]) in an aqueous EDTA-solution (133 millimol/L EDTA, 0.27% (w/v) bovine serum albumin in 66.7 millimol/L HEPES pH 7.5).

The resulting mixture was incubated 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm was measured in a TR-FRET reader, e.g. a Pherastar FS (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 micromol/L to 0.07 nanomol/L (20 micromol/L, 5.7 micromol/L, 1.6 micromol/L, 0.47 micromol/L, 0.13 micromol/L, 38 nanomol/L, 11 nanomol/L, 3.1 nanomol/L, 0.9 nanomol/L, 0.25 nanomol/L and 0.07 nanomol/L, the dilution series prepared separately before the assay on the level of the 100 fold concentrated solutions in DMSO by serial dilutions, exact concentrations may vary depending pipettors used) in duplicate values for each concentration and IC₅₀ values were calculated using Genedata Screener™ software.

TABLE 1
CDK12/CyclinK, 2 mM ATP (high ATP), IC₅₀-[mol/l] (median)

| | CDK12/CyclinK, 2 mM |
|---|---|
| Example | ATP (high ATP), IC50- |
| No | [mol/l] (median) |
| 1 | 5.97E−6 |
| 2 | >2.00E−5 |
| 3 | >2.00E−5 |
| 4 | 1.74E−6 |
| 5 | 5.17E−6 |
| 6 | 1.78E−6 |
| 7 | 1.02E−5 |
| 8 | >1.98E−5 |
| 9 | >2.00E−5 |
| 10 | 8.75E−6 |
| 11 | 3.16E−6 |
| 12 | 1.70E−5 |
| 13 | >2.00E−5 |
| 14 | >1.64E−5 |
| 15 | 6.02E−6 |
| 16 | 6.94E−6 |
| 17 | 5.72E−6 |
| 18 | >1.57E−5 |
| 19 | 1.15E−5 |
| 20 | 1.75E−6 |
| 21 | 9.64E−6 |
| 22 | 4.00E−6 |
| 23 | 1.56E−6 |
| 24 | 4.39E−6 |
| 25 | 7.31E−7 |
| 26 | 3.21E−6 |
| 27 | 3.48E−6 |
| 28 | 3.32E−6 |
| 29 | 3.67E−6 |
| 30 | 2.95E−6 |
| 31 | 1.36E−5 |
| 32 | 1.85E−5 |
| 33 | 5.23E−6 |
| 34 | >5.71E−6 |
| 35 | 2.21E−6 |
| 36 | >2.00E−5 |
| 37 | >2.00E−5 |
| 38 | >5.71E−6 |
| 39 | 4.67E−6 |
| 40 | 2.44E−6 |
| 41 | >5.71E−6 |
| 42 | 4.06E−7 |
| 43 | >1.68E−5 |
| 44 | 1.34E−6 |
| 45 | 2.20E−6 |
| 46 | 3.33E−6 |
| 47 | 7.19E−6 |
| 48 | >5.71E−6 |
| 49 | 6.12E−6 |
| 50 | 2.80E−6 |
| 51 | 1.73E−6 |
| 52 | 1.64E−6 |
| 53 | 2.03E−6 |
| 54 | 5.65E−6 |
| 55 | 7.71E−7 |
| 56 | 5.31E−6 |
| 57 | 1.04E−6 |
| 58 | 6.44E−6 |
| 59 | 5.47E−7 |
| 60 | 1.86E−6 |
| 61 | >2.00E−5 |
| 62 | 5.69E−6 |
| 63 | 1.15E−5 |
| 64 | 5.52E−6 |
| 65 | >5.71E−6 |
| 66 | 1.02E−6 |
| 67 | 2.74E−6 |
| 68 | 5.52E−6 |
| 69 | >5.71E−6 |
| 70 | 6.21E−7 |
| 71 | 4.33E−6 |
| 72 | 5.47E−7 |
| 73 | 6.64E−7 |
| 74 | >2.00E−5 |
| 75 | >3.77E−6 |
| 76 | >5.33E−6 |
| 77 | 1.01E−5 |
| 78 | 7.26E−7 |
| 79 | 5.66E−7 |
| 80 | 1.18E−6 |
| 81 | 7.65E−7 |
| 82 | 9.16E−7 |
| 83 | 1.04E−6 |
| 84 | 2.09E−6 |
| 85 | 6.33E−6 |
| 86 | 1.89E−6 |
| 87 | 2.59E−6 |
| 88 | 1.36E−6 |
| 89 | 1.07E−5 |
| 90 | 4.42E−6 |
| 91 | >5.71E−5 |
| 92 | 3.25E−6 |
| 93 | 2.70E−6 |
| 94 | 8.79E−6 |
| 95 | >2.00E−5 |
| 96 | 4.65E−6 |
| 97 | 7.88E−7 |
| 98 | 1.08E−6 |
| 99 | 1.54E−6 |
| 100 | 1.24E−6 |
| 101 | 2.25E−6 |
| 102 | 6.92E−6 |
| 103 | 1.03E−5 |
| 104 | >2.00E−5 |
| 105 | 1.07E−6 |
| 106 | >9.99E−6 |
| 107 | >2.00E−5 |
| 108 | >2.00E−5 |
| 109 | 1.76E−7 |
| 110 | 4.05E−7 |
| 111 | >1.63E−6 |
| 112 | 6.68E−06 |
| 113 | 1.71E−06 |
| 114 | 8.13E−06 |
| 115 | 1.16E−05 |
| 116 | 6.73E−06 |
| 117 | 4.82E−06 |
| 118 | 5.70E−06 |
| 119 | 6.62E−06 |
| 120 | 1.32E−05 |
| 121 | 7.47E−06 |
| 122 | >2.00E−5 |
| 123 | 1.26E−05 |
| 124 | 4.89E−07 |
| 125 | 1.09E−05 |
| 126 | 6.83E−06 |
| 127 | 9.71E−06 |
| 128 | 1.09E−05 |
| 129 | >1.82E−5 |
| 130 | 8.44E−06 |
| 131 | 1.11E−05 |
| 132 | >1.92E−5 |
| 133 | 7.01E−06 |
| 134 | >1.77E−5 |
| 135 | 1.45E−05 |
| 136 | 4.12E−06 |
| 137 | 1.30E−05 |
| 138 | >2.00E−5 |
| 139 | 1.53E−05 |
| 140 | 8.15E−06 |
| 141 | 1.31E−06 |
| 142 | 1.08E−05 |
| 143 | >2.00E−5 |
| 144 | 3.72E−06 |
| 145 | 1.26E−05 |
| 146 | 8.86E−06 |
| 147 | 4.97E−06 |
| 148 | 1.08E−05 |
| 149 | 6.22E−06 |
| 150 | 2.66E−06 |
| 151 | 4.24E−06 |
| 152 | 2.99E−06 |
| 153 | 2.95E−06 |
| 154 | 7.17E−06 |
| 155 | 3.74E−06 |
| 156 | 9.40E−06 |
| 157 | 6.76E−07 |
| 158 | 4.46E−06 |
| 159 | 9.62E−06 |
| 160 | 1.24E−06 |
| 161 | 9.61E−06 |
| 162 | 1.02E−06 |
| 163 | >5.71E−6 |
| 164 | >2.00E−5 |
| 165 | 1.02E−05 |
| 166 | 6.09E−06 |
| 167 | 7.12E−06 |
| 168 | 5.78E−06 |
| 169 | 1.37E−05 |
| 170 | 3.04E−06 |
| 171 | 6.56E−06 |
| 172 | 6.41E−07 |
| 173 | >2.00E−5 |
| 174 | 1.19E−06 |
| 175 | 1.10E−05 |
| 176 | 2.33E−06 |
| 177 | >2.00E−5 |
| 178 | >2.00E−5 |
| 179 | >2.00E−5 |
| 180 | 1.73E−05 |
| 181 | 1.91E−06 |
| 182 | 2.04E−06 |
| 183 | 1.02E−05 |
| 184 | 1.32E−08 |
| 185 | >1.96E−5 |
| 186 | 2.72E−06 |
| 187 | 6.50E−06 |
| 188 | 5.81E−07 |
| 189 | 1.09E−08 |
| 190 | 6.17E−06 |
| 191 | 3.32E−07 |
| 192 | 8.50E−06 |
| 193 | 1.75E−06 |
| 194 | 3.70E−06 |
| 195 | 8.23E−06 |
| 196 | 1.54E−05 |
| 197 | 9.53E−06 |
| 198 | 7.48E−06 |
| 199 | 1.48E−05 |
| 200 | 4.33E−06 |
| 201 | >2.00E−5 |
| 202 | 1.65E−05 |
| 203 | 8.80E−06 |
| 204 | 4.34E−06 |
| 205 | 7.97E−06 |
| 206 | 3.02E−07 |
| 207 | 7.69E−06 |
| 208 | 1.41E−05 |
| 209 | 6.80E−06 |
| 210 | >2.00E−5 |
| 211 | 2.74E−06 |
| 212 | 4.15E−06 |
| 213 | 1.05E−06 |
| 214 | 8.09E−06 |
| 215 | >2.00E−5 |
| 216 | >4.59E−6 |
| 217 | >2.00E−5 |
| 218 | >2.00E−5 |
| 219 | >2.00E−5 |
| 220 | 5.55E−06 |
| 221 | 7.98E−06 |
| 222 | 6.01E−06 |
| 223 | >1.65E−5 |
| 224 | 1.56E−05 |
| 225 | 1.03E−05 |
| 226 | >2.00E−5 |
| 227 | 9.51E−06 |
| 228 | >2.00E−5 |
| 229 | 5.51E−06 |
| 230 | >2.00E−5 |
| 231 | >2.00E−5 |
| 232 | 5.28E−06 |
| 233 | 7.54E−06 |
| 234 | 4.86E−06 |
| 235 | >2.00E−5 |
| 236 | >1.79E−5 |
| 237 | 7.35E−06 |
| 238 | 1.79E−06 |
| 239 | >2.00E−5 |
| 240 | >2.00E−5 |
| 241 | 8.05E−07 |
| 242 | >2.00E−5 |
| 243 | >2.00E−5 |
| 244 | >2.00E−5 |
| 245 | >1.58E−5 |
| 246 | >1.29E−5 |
| 247 | 1.47E−05 |
| 248 | 9.20E−06 |
| 249 | >1.70E−5 |
| 250 | 1.13E−05 |
| 251 | 9.72E−07 |
| 252 | >2.00E−5 |
| 253 | >2.00E−5 |
| 254 | >2.00E−5 |
| 255 | >2.00E−5 |
| 256 | >2.00E−5 |
| 257 | 1.17E−07 |
| 258 | 4.69E−06 |
| 259 | 2.59E−06 |
| 260 | 1.22E−05 |
| 261 | >1.87E−5 |
| 262 | >5.71E−6 |
| 263 | 6.89E−06 |
| 264 | 2.23E−06 |
| 265 | >1.67E−5 |
| 266 | 3.48E−06 |
| 267 | >2.00E−5 |
| 268 | 2.13E−06 |
| 269 | 8.61E−06 |
| 270 | 1.37E−05 |
| 271 | >2.00E−5 |
| 272 | >2.00E−5 |
| 273 | 9.83E−07 |
| 274 | 3.95E−08 |
| 275 | 9.73E−08 |
| 276 | >2.00E−5 |
| 277 | 4.79E−06 |
| 278 | 2.44E−06 |
| 279 | 1.40E−05 |
| 280 | 1.45E−05 |
| 281 | >1.69E−5 |
| 282 | >8.95E−6 |
| 283 | 1.83E−05 |
| 284 | 5.46E−06 |
| 285 | 6.44E−06 |
| 286 | 1.13E−05 |
| 287 | 1.07E−05 |
| 288 | 3.38E−06 |
| 289 | 7.00E−06 |
| 290 | >1.98E−5 |
| 291 | 1.07E−05 |
| 292 | 6.66E−07 |
| 293 | 5.91E−06 |
| 294 | 1.86E−06 |
| 295 | 8.29E−07 |
| 296 | 2.48E−06 |
| 297 | 1.74E−05 |
| 298 | 8.31E−06 |
| 299 | 6.39E−06 |
| 300 | >2.00E−5 |
| 301 | 1.18E−06 |
| 302 | 5.96E−06 |
| 303 | 1.71E−06 |
| 304 | 1.25E−05 |
| 305 | 1.08E−05 |
| 306 | 1.85E−06 |
| 307 | 1.57E−06 |
| 308 | 6.40E−06 |
| 309 | >2.00E−5 |
| 310 | 1.29E−06 |
| 311 | >2.00E−5 |
| 312 | 1.02E−05 |
| 313 | 1.82E−05 |
| 314 | 9.92E−06 |
| 315 | >1.97E−5 |
| 316 | 2.92E−6 |
| 317 | 9.79E−7 |
| 318 | 1.36E−6 |
| 319 | 2.38E−6 |
| 320 | 5.57E−7 |
| 321 | 4.16E−7 |
| 322 | 1.21E−6 |
| 323 | 2.85E−6 |
| 324 | 8.59E−7 |
| 325 | 3.55E−6 |
| 326 | 9.42E−7 |
| 327 | 2.35E−6 |
| 328 | 1.66E−6 |
| 329 | 5.40E−7 |
| 330 | 4.63E−6 |
| 331 | 3.50E−7 |
| 332 | 1.90E−7 |
| 333 | 1.42E−7 |
| 334 | 2.03E−6 |
| 335 | 6.65E−6 |
| 336 | 7.89E−7 |
| 337 | 8.72E−6 |
| 338 | 1.18E−6 |
| 339 | 1.55E−6 |
| 340 | 6.18E−7 |
| 341 | 2.22E−6 |
| 342 | 6.65E−6 |
| 343 | 1.88E−7 |
| 344 | 8.37E−6 |
| 345 | 6.61E−7 |
| 346 | 1.22E−5 |
| 347 | 2.18E−6 |
| 348 | 2.89E−6 |
| 349 | 8.67E−6 |
| 350 | 7.80E−6 |
| 351 | 9.35E−6 |
| 352 | 5.25E−6 |
| 353 | 2.52E−6 |
| 354 | 3.74E−6 |
| 355 | 4.18E−6 |
| 356 | 4.91E−6 |
| 357 | 1.15E−6 |
| 358 | 1.16E−5 |
| 359 | 2.09E−6 |
| 360 | 4.01E−6 |
| 361 | 1.43E−6 |
| 362 | 1.31E−5 |
| 363 | 3.31E−6 |
| 364 | 6.67E−6 |
| 365 | 5.85E−6 |
| 366 | 1.81E−6 |
| 367 | 2.93E−6 |
| 368 | 4.84E−6 |
| 369 | >2.00E−5 |
| 370 | 1.69E−7 |
| 371 | 5.25E−6 |
| 372 | 2.69E−7 |
| 373 | 3.06E−6 |
| 374 | 1.55E−7 |
| 375 | 4.56E−6 |
| 376 | >2.00E−5 |
| 377 | 5.09E−6 |
| 378 | 4.40E−7 |
| 379 | 5.28E−7 |
| 380 | 1.02E−6 |
| 381 | 5.91E−6 |
| 382 | 8.23E−7 |
| 383 | 4.97E−6 |
| 384 | 8.11E−6 |
| 385 | 2.80E−7 |
| 386 | 1.15E−7 |
| 387 | 2.33E−7 |
| 388 | 6.84E−6 |
| 389 | >2.00E−5 |
| 390 | >2.00E−5 |
| 391 | 6.70E−6 |
| 392 | 4.37E−6 |
| 393 | >2.00E−5 |
| 394 | >1.87E−5 |
| 395 | 6.40E−6 |
| 396 | >2.00E−5 |
| 397 | >1.75E−5 |
| 398 | 1.46E−5 |
| 399 | >2.00E−5 |
| 400 | >1.67E−5 |
| 401 | >2.00E−5 |
| 402 | 2.24E−7 |
| 403 | >2.00E−5 |
| 404 | >2.00E−5 |
| 405 | >2.00E−5 |
| 406 | >2.00E−5 |
| 407 | 7.06E−7 |
| 408 | >2.00E−5 |
| 409 | >1.58E−5 |
| 410 | 1.58E−6 |
| 411 | >2.00E−5 |
| 412 | >5.71E−6 |
| 413 | >2.00E−5 |
| 414 | >5.71E−6 |
| 415 | >2.00E−5 |
| 416 | >2.00E−5 |
| 417 | >7.75E−6 |
| 418 | >2.00E−5 |
| 419 | >2.00E−5 |
| 420 | >2.00E−5 |
| 421 | 8.58E−6 |
| 422 | 1.63E−6 |
| 423 | >1.29E−5 |
| 424 | >2.00E−5 |
| 425 | 2.14E−6 |
| 426 | 2.39E−8 |
| 427 | 1.86E−6 |
| 428 | >2.00E−5 |
| 429 | >2.00E−5 |
| 430 | >2.00E−5 |
| 431 | 2.81E−6 |
| 432 | 2.30E−6 |
| 433 | 2.70E−6 |
| 434 | >1.76E−5 |
| 435 | 5.71E−6 |
| 436 | >2.00E−5 |
| 437 | >2.00E−5 |
| 438 | >7.86E−6 |
| 439 | 3.95E−6 |
| 440 | >2.00E−5 |
| 441 | 4.18E−6 |
| 442 | 5.83E−7 |
| 443 | 3.98E−6 |
| 444 | 8.51E−6 |

### 2.3 CDK13/CycK Low ATP Kinase Assay

CDK13/CycK-inhibitory activity of compounds of the present invention at 10 micromol/L adenosine-tri-phosphate (ATP) was quantified employing the TR-FRET (TR-FRET=Time Resolved Fluorescence Energy Transfer) based CDK13/CycK activity inhibition assay as described in the following paragraphs.

A complex of human recombinant CDK13 and human recombinant CycK (both N-terminally His-tagged, expression and purification as described above) was used as enzyme. As substrate for the kinase reaction biotinylated peptide biotin-Ahx-KFELLPTPPLSPSRRSGL (C-terminus in amid form) was used which can be purchased e.g. form the company Biosyntan (Berlin-Buch, Germany).

For the assay 50 nanoL of a 100 fold concentrated solution of the test compound in DMSO was pipetted into either a black low volume 384 well microtiter plate or a black 1536 well microtiter plate (both Greiner Bio-One, Frickenhausen, Germany), 2 microL of a solution of CDK13/CycK in aqueous assay buffer [25 millimol/L HEPES pH 7.5, 20 millimol/L MgCl₂, 5 millimol/L β-glycerophosphate, 2 millimol/L EGTA, 1.0 millimol/L dithiothreitol, 0.01% (v/v) Nonidet-P40 (Sigma), 0.01% (w/v) bovine serum albumin] were added and the mixture was incubated for 15 min at 22° C. to allow pre-binding of the test compounds to the enzyme before the start of the kinase reaction. Then the kinase reaction was started by the addition of 3 microL of a solution ATP (16.7 micromol/L=>final conc. in the 5 microL assay volume is 10 micromol/L) and substrate (1.67 micromol/L=>final conc. in the 5 microL assay volume is 1 micromol/L) in assay buffer and the resulting mixture was incubated for a reaction time of 60 min at 22° C. The concentration of CDK13/CycK was adjusted depending of the activity of the enzyme lot and was chosen appropriate to have the assay in the linear range, typical concentrations were about 5 nanomol/L. The reaction was stopped by the addition of 3 microL of a solution of TR-FRET detection reagents (125 nanomol/L streptavidine-XL665 [Cisbio Bioassays, Codolet, France] and 0.67 nanomol/L anti-Phospho-c-Myc (Ser 62) (E1J4K)-antibody from Cell Signalling [#13748] and 2 nanomol/L LANCE EU-W1024 labeled anti-rabbit IgG antibody [Perkin-Elmer, product no. 0083]) in an aqueous EDTA-solution (133 millimol/L EDTA, 0.27% (w/v) bovine serum albumin in 66.7 millimol/L HEPES pH 7.5).

The resulting mixture was incubated 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm was measured in a TR-FRET reader, e.g. a Pherastar FS (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 micromol/L to 0.07 nanomol/L (20 micromol/L, 5.7 micromol/L, 1.6 micromol/L, 0.47 micromol/L, 0.13 micromol/L, 38 nanomol/L, 11 nanomol/L, 3.1 nanomol/L, 0.9 nanomol/L, 0.25 nanomol/L and 0.07 nanomol/L, the dilution series prepared separately before the assay on the level of the 100 fold concentrated solutions in DMSO by serial dilutions, exact concentrations may vary depending pipettors used) in duplicate values for each concentration and IC₅₀ values were calculated using Genedata Screener™ software.

### 2.4 CDK2/CycE Kinase Assay

CDK2/CycE-inhibitory activity of compounds of the present invention was quantified employing the CDK2/CycE TR-FRET assay as described in the following paragraphs.

Recombinant fusion proteins of GST and human CDK2 and of GST and human CycE, expressed in insect cells (Sf9) and purified by Glutathion-Sepharose affinity chromatography, were purchase from ProQinase GmbH (Freiburg, Germany). As substrate for the kinase reaction biotinylated peptide biotin-Ttds-YISPLKSPYKISEG (C-terminus in amid form) was used which can be purchased e.g. form the company JERINI peptide technologies (Berlin, Germany).

For the assay 50 nanoL of a 100 fold concentrated solution of the test compound in DMSO was pipetted into a black low volume 384 well microtiter plate or a black 1536 well microtiter plate (both Greiner Bio-One, Frickenhausen, Germany), 2 microL of a solution of CDK2/CycE in aqueous assay buffer [50 millimol/L Tris/HCl pH 8.0, 10 millimol/L MgCl₂, 1.0 millimol/L dithiothreitol, 0.1 millimol/L sodium ortho-vanadate, 0.01% (v/v) Nonidet-P40 (Sigma)] were added and the mixture was incubated for 15 min at 22° C. to allow pre-binding of the test compounds to the enzyme before the start of the kinase reaction. Then the kinase reaction was started by the addition of 3 microL of a solution of adenosine-tri-phosphate (ATP, 3.33 millimol/L=>final conc. in the 5 microL assay volume is 2 millimol/L) and substrate (1.25 micromol/L=>final conc. in the 5 microL assay volume is 0.75 micromol/L) in assay buffer and the resulting mixture was incubated for a reaction time of 25 min at 22° C. The concentration of CDK2/CycE was adjusted depending of the activity of the enzyme lot and was chosen appropriate to have the assay in the linear range, typical concentrations were in the range of 10 ng/ml. The reaction was stopped by the addition of 3 microL of a solution of TR-FRET detection reagents (0.333 micromol/L streptavidine-XL665 [Cisbio Bioassays, Codolet, France] and 1.67 nanomol/L anti-RB(pSer807/pSer8l 1)-antibody from BD Pharmingen [#558389] and 2 nanomol/L LANCE EU-W1024 labeled anti-mouse IgG antibody [Perkin-Elmer, product no. AD0077, as an alternative a Terbium-cryptate-labeled anti-mouse IgG antibody from Cisbio Bioassays can be used]) in an aqueous EDTA-solution (167 millimol/L EDTA, 0.2% (w/v) bovine serum albumin in 100 millimol/L HEPES pH 7.5).

The resulting mixture was incubated 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm was measured in a TR-FRET reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 micromol/L to 0.07 nanomol/L (20 micromol/L, 5.7 micromol/L, 1.6 micromol/L, 0.47 micromol/L, 0.13 micromol/L, 38 nanomol/L, 11 nanomol/L, 3.1 nanomol/L, 0.9 nanomol/L, 0.25 nanomol/L and 0.07 nanomol/L, the dilution series prepared separately before the assay on the level of the 100 fold concentrated solutions in DMSO by serial dilutions, exact concentrations may vary depending pipettors used) in duplicate values for each concentration and IC50 values were calculated using Genedata Screener™ software.

TABLE 2
CDK2/CyclinE, 2 mM ATP (high ATP), IC₅₀-[mol/l]

| | CDK2/CyclinE, 2 mM ATP |
|---|---|
| Example | (high ATP), IC50-[mol/l] |
| No | (median) |
| 1 | 5.27E−6 |
| 2 | 1.11E−5 |
| 3 | >2.00E−5 |
| 4 | 2.28E−6 |
| 5 | 1.36E−6 |
| 6 | 2.01E−6 |
| 7 | 6.57E−6 |
| 8 | 1.11E−5 |
| 9 | 1.47E−5 |
| 10 | 8.74E−7 |
| 11 | 5.51E−6 |
| 12 | >1.65E−5 |
| 13 | >2.00E−5 |
| 14 | >2.00E−5 |
| 15 | 6.52E−6 |
| 16 | 1.50E−6 |
| 17 | 2.89E−6 |
| 18 | 6.29E−6 |
| 19 | 1.14E−5 |
| 20 | 1.72E−6 |
| 21 | 2.07E−6 |
| 22 | 1.55E−5 |
| 23 | 1.41E−6 |
| 24 | 2.32E−6 |
| 25 | 4.56E−6 |
| 26 | 2.07E−6 |
| 27 | 1.71E−5 |
| 28 | 6.02E−6 |
| 29 | 3.08E−6 |
| 30 | 7.28E−6 |
| 31 | 1.75E−5 |
| 32 | 1.56E−5 |
| 33 | 8.26E−6 |
| 34 | 6.18E−6 |
| 35 | 1.70E−6 |
| 36 | >2.00E−5 |
| 37 | >1.53E−5 |
| 38 | 8.30E−6 |
| 39 | 2.11E−6 |
| 40 | 2.11E−6 |
| 41 | >1.63E−6 |
| 42 | 1.03E−6 |
| 43 | >2.00E−5 |
| 44 | 1.13E−6 |
| 45 | 5.37E−6 |
| 46 | 1.85E−6 |
| 47 | 4.66E−6 |
| 48 | 1.26E−5 |
| 49 | 2.29E−6 |
| 50 | 2.33E−6 |
| 51 | 1.64E−6 |
| 52 | 2.65E−6 |
| 53 | 1.68E−6 |
| 54 | 1.89E−6 |
| 55 | 1.07E−6 |
| 56 | 2.02E−6 |
| 57 | 1.22E−6 |
| 58 | 1.07E−5 |
| 59 | 1.05E−6 |
| 60 | 7.38E−7 |
| 61 | 9.06E−6 |
| 62 | 6.45E−6 |
| 63 | 6.49E−6 |
| 64 | 4.66E−6 |
| 65 | >2.00E−5 |
| 66 | 2.30E−6 |
| 67 | 1.86E−6 |
| 68 | 1.06E−5 |
| 69 | 1.21E−5 |
| 70 | 1.54E−6 |
| 71 | 2.23E−6 |
| 72 | 4.35E−6 |
| 73 | 6.09E−6 |
| 74 | 1.82E−5 |
| 75 | 2.50E−6 |
| 76 | 8.34E−6 |
| 77 | 1.11E−5 |
| 78 | 4.28E−6 |
| 79 | 2.30E−6 |
| 80 | 4.04E−6 |
| 81 | 4.03E−6 |
| 82 | 1.16E−5 |
| 83 | 7.66E−6 |
| 84 | 4.84E−6 |
| 85 | 4.52E−6 |
| 86 | 1.95E−6 |
| 87 | 2.55E−6 |
| 88 | 8.55E−6 |
| 89 | 9.87E−6 |
| 90 | 1.79E−6 |
| 91 | >2.00E−5 |
| 92 | 1.56E−5 |
| 93 | 1.05E−5 |
| 94 | >2.00E−5 |
| 95 | >2.00E−5 |
| 96 | 1.78E−6 |
| 97 | 3.24E−6 |
| 98 | 2.03E−6 |
| 99 | 1.77E−6 |
| 100 | 1.83E−6 |
| 101 | 3.88E−6 |
| 102 | 2.42E−6 |
| 103 | 9.05E−6 |
| 104 | >2.00E−5 |
| 105 | 8.26E−7 |
| 106 | >2.00E−5 |
| 107 | >2.00E−5 |
| 108 | >2.00E−5 |
| 109 | 4.01E−6 |
| 110 | 8.21E−6 |
| 111 | >2.00E−5 |
| 112 | 4.34E−06 |
| 113 | 1.20E−06 |
| 114 | 5.44E−06 |
| 115 | 1.07E−05 |
| 116 | 3.01E−06 |
| 117 | 4.66E−06 |
| 118 | 3.39E−06 |
| 119 | 7.77E−06 |
| 120 | 1.54E−05 |
| 121 | 2.20E−06 |
| 122 | 6.72E−06 |
| 123 | >2.00E−5 |
| 124 | 8.97E−06 |
| 125 | 1.32E−05 |
| 126 | 5.99E−06 |
| 127 | >2.00E−5 |
| 128 | 9.63E−06 |
| 129 | >2.00E−5 |
| 130 | 8.15E−06 |
| 131 | >2.00E−5 |
| 132 | 7.43E−06 |
| 133 | 1.51E−05 |
| 134 | 1.20E−05 |
| 135 | 7.75E−06 |
| 136 | 3.45E−06 |
| 137 | 1.53E−05 |
| 138 | >2.00E−5 |
| 139 | 1.47E−05 |
| 140 | 1.25E−06 |
| 141 | 7.07E−07 |
| 142 | >1.98E−5 |
| 143 | 1.78E−05 |
| 144 | 7.81E−06 |
| 145 | 6.14E−06 |
| 146 | 7.11E−06 |
| 147 | 3.12E−06 |
| 148 | 1.26E−05 |
| 149 | 6.38E−06 |
| 150 | 4.36E−06 |
| 151 | 1.34E−05 |
| 152 | 5.42E−06 |
| 153 | 1.70E−06 |
| 154 | 4.47E−06 |
| 155 | 9.28E−06 |
| 156 | 3.48E−06 |
| 157 | 3.02E−06 |
| 158 | 5.30E−06 |
| 159 | >2.00E−5 |
| 160 | 5.17E−06 |
| 161 | 1.65E−05 |
| 162 | 4.63E−06 |
| 163 | 1.08E−05 |
| 164 | >2.00E−5 |
| 165 | >2.00E−5 |
| 166 | 4.70E−06 |
| 167 | 1.59E−05 |
| 168 | 1.23E−05 |
| 169 | 1.33E−05 |
| 170 | >2.00E−5 |
| 171 | >2.00E−5 |
| 172 | >2.00E−5 |
| 173 | >2.00E−5 |
| 174 | >2.00E−5 |
| 175 | >2.00E−5 |
| 176 | 8.60E−06 |
| 177 | >2.00E−5 |
| 178 | >2.00E−5 |
| 179 | >2.00E−5 |
| 180 | >2.00E−5 |
| 181 | >2.00E−5 |
| 182 | >2.00E−5 |
| 183 | >2.00E−5 |
| 184 | 2.72E−06 |
| 185 | >2.00E−5 |
| 186 | >2.00E−5 |
| 187 | >2.00E−5 |
| 188 | 1.50E−05 |
| 189 | 7.61E−07 |
| 190 | >2.00E−5 |
| 191 | 1.77E−06 |
| 192 | 4.17E−06 |
| 193 | 1.08E−05 |
| 194 | >2.00E−5 |
| 195 | >2.00E−5 |
| 196 | >1.98E−5 |
| 197 | 1.34E−05 |
| 198 | 9.94E−06 |
| 199 | 5.17E−06 |
| 200 | 4.63E−06 |
| 201 | >2.00E−5 |
| 202 | 1.05E−05 |
| 203 | 9.30E−06 |
| 204 | 1.09E−05 |
| 205 | >2.00E−5 |
| 206 | 4.04E−06 |
| 207 | 1.03E−05 |
| 208 | 1.85E−05 |
| 209 | >2.00E−5 |
| 210 | >2.00E−5 |
| 211 | 8.56E−06 |
| 212 | >1.90E−5 |
| 213 | 5.49E−06 |
| 214 | 1.77E−05 |
| 215 | 8.98E−06 |
| 216 | 1.52E−05 |
| 217 | >2.00E−5 |
| 218 | 5.78E−06 |
| 219 | >2.00E−5 |
| 220 | 9.08E−07 |
| 221 | 5.01E−06 |
| 222 | 1.46E−05 |
| 223 | 1.44E−05 |
| 224 | 7.75E−06 |
| 225 | 5.39E−06 |
| 226 | >2.00E−5 |
| 227 | 1.87E−06 |
| 228 | 9.21E−06 |
| 229 | 1.46E−06 |
| 230 | 1.01E−05 |
| 231 | 1.24E−05 |
| 232 | 2.87E−06 |
| 233 | 5.35E−06 |
| 234 | 2.73E−06 |
| 235 | >2.00E−5 |
| 236 | 1.28E−05 |
| 237 | 4.94E−06 |
| 238 | 1.86E−06 |
| 239 | >2.00E−5 |
| 240 | 1.55E−05 |
| 241 | 1.20E−05 |
| 242 | >2.00E−5 |
| 243 | >2.00E−5 |
| 244 | >2.00E−5 |
| 245 | 5.00E−06 |
| 246 | >2.00E−5 |
| 247 | >2.00E−5 |
| 248 | >2.00E−5 |
| 249 | >2.00E−5 |
| 250 | 1.28E−05 |
| 251 | 2.58E−06 |
| 252 | 1.33E−05 |
| 253 | 1.22E−05 |
| 254 | >2.00E−5 |
| 255 | >2.00E−5 |
| 256 | 5.06E−06 |
| 257 | 1.61E−05 |
| 258 | 3.10E−06 |
| 259 | 1.40E−06 |
| 260 | 2.98E−06 |
| 261 | 7.29E−06 |
| 262 | >2.00E−5 |
| 263 | >2.00E−5 |
| 264 | >2.00E−5 |
| 265 | >2.00E−5 |
| 266 | 2.10E−06 |
| 267 | 1.61E−05 |
| 268 | >2.00E−5 |
| 269 | 4.84E−06 |
| 270 | 1.79E−06 |
| 271 | 1.00E−05 |
| 272 | 1.62E−05 |
| 273 | 1.86E−05 |
| 274 | >1.29E−5 |
| 275 | >2.00E−5 |
| 276 | >2.00E−5 |
| 277 | >2.00E−5 |
| 278 | >2.00E−5 |
| 279 | >2.00E−5 |
| 280 | 1.74E−05 |
| 281 | 3.28E−06 |
| 282 | 1.02E−05 |
| 283 | 7.62E−06 |
| 284 | 1.99E−06 |
| 285 | 2.55E−06 |
| 286 | >2.00E−5 |
| 287 | 9.48E−06 |
| 288 | 2.64E−06 |
| 289 | 5.29E−06 |
| 290 | 7.21E−06 |
| 291 | 4.07E−06 |
| 292 | >5.19E−6 |
| 293 | 1.29E−05 |
| 294 | 1.72E−05 |
| 295 | 2.06E−06 |
| 296 | 5.61E−06 |
| 297 | 1.33E−05 |
| 298 | >1.95E−5 |
| 299 | >2.00E−5 |
| 300 | >1.97E−5 |
| 301 | >2.00E−5 |
| 302 | >2.00E−5 |
| 303 | >2.00E−5 |
| 304 | 8.43E−06 |
| 305 | >2.00E−5 |
| 306 | >2.00E−5 |
| 307 | >2.00E−5 |
| 308 | >2.00E−5 |
| 309 | >2.00E−5 |
| 310 | >1.86E−5 |
| 311 | >2.00E−5 |
| 312 | >1.25E−5 |
| 313 | >2.00E−5 |
| 314 | >2.00E−5 |
| 315 | >2.00E−5 |
| 316 | 2.84E−7 |
| 317 | 1.07E−5 |
| 318 | >2.00E−5 |
| 319 | 1.21E−5 |
| 320 | 4.71E−6 |
| 321 | 1.00E−6 |
| 322 | >2.00E−5 |
| 323 | 1.35E−5 |
| 324 | 1.38E−5 |
| 325 | >2.00E−5 |
| 326 | 4.14E−6 |
| 327 | >1.87E−5 |
| 328 | 1.45E−5 |
| 329 | 9.46E−6 |
| 330 | 1.38E−5 |
| 331 | 7.31E−6 |
| 332 | >2.00E−5 |
| 333 | >2.00E−5 |
| 334 | 1.05E−5 |
| 335 | 2.80E−6 |
| 336 | 8.95E−6 |
| 337 | 1.78E−5 |
| 338 | 6.57E−6 |
| 339 | 6.74E−6 |
| 340 | 1.16E−6 |
| 341 | 9.36E−6 |
| 342 | 2.94E−6 |
| 343 | 1.19E−6 |
| 344 | 1.24E−5 |
| 345 | 6.07E−6 |
| 346 | >2.00E−5 |
| 347 | 9.08E−6 |
| 348 | 1.32E−5 |
| 349 | 1.82E−6 |
| 350 | 3.39E−6 |
| 351 | 7.39E−6 |
| 352 | 2.30E−6 |
| 353 | 2.86E−6 |
| 354 | 1.53E−5 |
| 355 | >1.86E−5 |
| 356 | >2.00E−5 |
| 357 | 1.19E−5 |
| 358 | >2.00E−5 |
| 359 | 1.24E−5 |
| 360 | 1.21E−5 |
| 361 | 4.34E−6 |
| 362 | 9.31E−6 |
| 363 | 8.61E−6 |
| 364 | 2.07E−6 |
| 365 | 1.74E−6 |
| 366 | 5.57E−6 |
| 367 | 5.73E−7 |
| 368 | 8.85E−7 |
| 369 | 1.05E−5 |
| 370 | >2.00E−5 |
| 371 | >2.00E−5 |
| 372 | 3.65E−6 |
| 373 | 7.50E−6 |
| 374 | 3.12E−6 |
| 375 | 1.19E−5 |
| 376 | >2.00E−5 |
| 377 | 1.71E−5 |
| 378 | 8.11E−6 |
| 379 | 1.43E−5 |
| 380 | 9.26E−6 |
| 381 | >2.00E−5 |
| 382 | 1.42E−5 |
| 383 | 3.95E−6 |
| 384 | >2.00E−5 |
| 385 | 1.81E−6 |
| 386 | 1.45E−6 |
| 387 | 5.35E−6 |
| 388 | >2.00E−5 |
| 389 | >2.00E−5 |
| 390 | >2.00E−5 |
| 391 | 4.91E−6 |
| 392 | >2.00E−5 |
| 393 | 9.94E−6 |
| 394 | >2.00E−5 |
| 395 | >2.00E−5 |
| 396 | 1.39E−5 |
| 397 | 1.72E−5 |
| 398 | 1.04E−5 |
| 399 | >2.00E−5 |
| 400 | >2.00E−5 |
| 401 | >2.00E−5 |
| 402 | 9.56E−6 |
| 403 | >2.00E−5 |
| 404 | >2.00E−5 |
| 405 | >2.00E−5 |
| 406 | 1.50E−5 |
| 407 | >2.00E−5 |
| 408 | >1.97E−5 |
| 409 | 1.28E−5 |
| 410 | 1.00E−5 |
| 411 | 1.63E−5 |
| 412 | >2.00E−5 |
| 413 | >2.00E−5 |
| 414 | >2.00E−5 |
| 415 | >2.00E−5 |
| 416 | >2.00E−5 |
| 417 | >2.00E−5 |
| 418 | >2.00E−5 |
| 419 | >2.00E−5 |
| 420 | >2.00E−5 |
| 421 | >2.00E−5 |
| 422 | >2.00E−5 |
| 423 | >2.00E−5 |
| 424 | >2.00E−5 |
| 425 | >2.00E−5 |
| 426 | 1.73E−5 |
| 427 | 5.23E−6 |
| 428 | >2.00E−5 |
| 429 | >2.00E−5 |
| 430 | >2.00E−5 |
| 431 | 1.59E−6 |
| 432 | 7.57E−6 |
| 433 | 1.78E−6 |
| 434 | 6.37E−6 |
| 435 | 5.00E−6 |
| 436 | >2.00E−5 |
| 437 | >2.00E−5 |
| 438 | >2.00E−5 |
| 439 | >2.00E−5 |
| 440 | >2.00E−5 |
| 441 | >2.00E−5 |
| 442 | >2.00E−5 |
| 443 | >2.00E−5 |
| 444 | 1.54E−5 |

### 2.5 CDK9/CycT1 High ATP Kinase Assay

CDK9/CycT1-inhibitory activity of compounds of the present invention at a high ATP concentration after preincubation of enzyme and test compounds was quantified employing the CDK9/CycT1 TR-FRET assay as described in the following paragraphs.

Recombinant full-length His-tagged human CDK9 and CycT1, expressed in insect cells and purified by Ni-NTA affinity chromatography, were purchased from Life Technologies (Cat. No PV4131). As substrate for the kinase reaction biotinylated peptide biotin-Ttds-YISPLKSPYKISEG (C-terminus in amide form) was used which can be purchased e.g. form the company JERINI peptide technologies (Berlin, Germany).

For the assay 50 nanoL of a 100 fold concentrated solution of the test compound in DMSO was pipetted into either a black low volume 384 well microtiter plate or a black 1536 well microtiter plate (both Greiner Bio-One, Frickenhausen, Germany), 2 microL of a solution of CDK9/CycT1 in aqueous assay buffer [50 millimol/L Tris/HCl pH 8.0, 10 millimol/L MgCl₂, 1.0 millimol/L dithiothreitol, 0.1 millimol/L sodium ortho-vanadate, 0.01% (v/v) Nonidet-P40 (Sigma)] were added and the mixture was incubated for 15 min at 22° C. to allow pre-binding of the test compounds to the enzyme before the start of the kinase reaction. Then the kinase reaction was started by the addition of 3 microL of a solution of adenosine-tri-phosphate (ATP, 3.3 millimol/L=>final conc. in the 5 microL assay volume is 2 millimol/L) and substrate (1.25 micromol/L=>final conc. in the 5 microL assay volume is 0.75 micromol/L) in assay buffer and the resulting mixture was incubated for a reaction time of 25 min at 22° C. The concentration of CDK9/CycT1 was adjusted depending of the activity of the enzyme lot and was chosen appropriate to have the assay in the linear range, typical concentrations were in the range of 0.5 microg/ml. The reaction was stopped by the addition of 3 microL of a solution of TR-FRET detection reagents (0.33 micromol/L streptavidine-XL665 [Cisbio Bioassays, Codolet, France] and 1.67 nanomol/L anti-RB(pSer807/pSer8l 1)-antibody from BD Pharmingen [#558389] and 2 nanomol/L LANCE EU-W1024 labeled anti-mouse IgG antibody [Perkin-Elmer, product no. AD0077]) in an aqueous EDTA-solution (167 millimol/L EDTA, 0.2% (w/v) bovine serum albumin in 100 millimol/L HEPES pH 7.5).

The resulting mixture was incubated 1 h at 22° C. to allow the formation of complex between the phosphorylated biotinylated peptide and the detection reagents. Subsequently the amount of phosphorylated substrate was evaluated by measurement of the resonance energy transfer from the Eu-chelate to the streptavidine-XL. Therefore, the fluorescence emissions at 620 nm and 665 nm after excitation at 350 nm was measured in a TR-FRET reader, e.g. a Pherastar (BMG Labtechnologies, Offenburg, Germany) or a Viewlux (Perkin-Elmer). The ratio of the emissions at 665 nm and at 622 nm was taken as the measure for the amount of phosphorylated substrate. The data were normalised (enzyme reaction without inhibitor=0% inhibition, all other assay components but no enzyme=100% inhibition). Usually the test compounds were tested on the same microtiterplate in 11 different concentrations in the range of 20 micromol/L to 0.07 nanomol/L (20 micromol/L, 5.7 micromol/L, 1.6 micromol/L, 0.47 micromol/L, 0.13 micromol/L, 38 nanomol/L, 11 nanomol/L, 3.1 nanomol/L, 0.9 nanomol/L, 0.25 nanomol/L and 0.07 nanomol/L, the dilution series prepared separately before the assay on the level of the 100 fold concentrated solutions in DMSO by serial dilutions, exact concentrations may vary depending pipettors used) in duplicate values for each concentration and IC₅₀-values were calculated using Genedata Screener™ software.

TABLE 3
CDK9/CyclinT1, 2 mM ATP (high ATP), IC₅₀-[mol/l]

| | CDK9/CyclinT1, 2 mM ATP |
|---|---|
| Example | (high ATP), IC50-[mol/l] |
| No | (median) |
| 1 | >2.00E−5 |
| 2 | >2.00E−5 |
| 3 | >2.00E−5 |
| 4 | >2.00E−5 |
| 5 | >2.00E−5 |
| 6 | >1.61E−5 |
| 7 | >2.00E−5 |
| 8 | >2.00E−5 |
| 9 | >2.00E−5 |
| 10 | >2.00E−5 |
| 11 | >2.00E−5 |
| 12 | >2.00E−5 |
| 13 | >2.00E−5 |
| 14 | >2.00E−5 |
| 15 | >2.00E−5 |
| 16 | >2.00E−5 |
| 17 | >2.00E−5 |
| 18 | >2.00E−5 |
| 19 | >2.00E−5 |
| 20 | 9.22E−6 |
| 21 | >2.00E−5 |
| 22 | >1.84E−5 |
| 23 | 3.24E−6 |
| 24 | 3.06E−6 |
| 25 | 5.78E−6 |
| 26 | 8.74E−6 |
| 27 | 1.47E−5 |
| 28 | >2.00E−5 |
| 29 | 2.40E−6 |
| 30 | >2.00E−5 |
| 31 | >2.00E−5 |
| 32 | >2.00E−5 |
| 33 | 1.41E−5 |
| 34 | >2.00E−5 |
| 35 | 9.15E−6 |
| 36 | >2.00E−5 |
| 37 | >2.00E−5 |
| 38 | >2.00E−5 |
| 39 | 4.91E−6 |
| 40 | 2.82E−6 |
| 41 | >2.00E−5 |
| 42 | 9.37E−7 |
| 43 | >2.00E−5 |
| 44 | 8.85E−7 |
| 45 | 3.41E−6 |
| 46 | >2.00E−5 |
| 47 | >2.00E−5 |
| 48 | >2.00E−5 |
| 49 | 2.61E−6 |
| 50 | 2.01E−6 |
| 51 | 1.29E−6 |
| 52 | 1.84E−6 |
| 53 | 1.29E−6 |
| 54 | 6.24E−6 |
| 55 | 7.18E−7 |
| 56 | >2.00E−5 |
| 57 | 6.67E−6 |
| 58 | >2.00E−5 |
| 59 | 1.07E−5 |
| 60 | 7.34E−6 |
| 61 | >2.00E−5 |
| 62 | >2.00E−5 |
| 63 | >2.00E−5 |
| 64 | 3.56E−6 |
| 65 | >5.71E−6 |
| 66 | 4.48E−6 |
| 67 | >1.94E−5 |
| 68 | 5.34E−6 |
| 69 | 8.35E−6 |
| 70 | 5.69E−7 |
| 71 | 1.76E−6 |
| 72 | 6.72E−6 |
| 73 | 9.38E−6 |
| 74 | >2.00E−5 |
| 75 | >6.15E−6 |
| 76 | 7.85E−6 |
| 77 | 1.29E−5 |
| 78 | 7.68E−7 |
| 79 | 2.53E−6 |
| 80 | 5.43E−6 |
| 81 | 5.39E−6 |
| 82 | 1.26E−5 |
| 83 | 6.79E−6 |
| 84 | 3.44E−6 |
| 85 | >1.08E−5 |
| 86 | 5.27E−6 |
| 87 | 5.13E−6 |
| 88 | 6.42E−6 |
| 89 | >2.00E−5 |
| 90 | 9.35E−6 |
| 91 | >2.00E−5 |
| 92 | >2.00E−5 |
| 93 | 8.59E−6 |
| 94 | 1.28E−5 |
| 95 | >2.00E−5 |
| 96 | 9.30E−6 |
| 97 | 1.34E−5 |
| 98 | 9.53E−7 |
| 99 | 9.70E−7 |
| 100 | 8.20E−7 |
| 101 | >2.00E−5 |
| 102 | >2.00E−5 |
| 103 | >2.00E−5 |
| 104 | >2.00E−5 |
| 105 | 6.13E−7 |
| 106 | >2.00E−5 |
| 107 | >2.00E−5 |
| 108 | >2.00E−5 |
| 109 | 1.79E−6 |
| 110 | 5.47E−6 |
| 111 | >1.63E−6 |
| 112 | >2.00E−5 |
| 113 | >1.89E−5 |
| 114 | >2.00E−5 |
| 115 | >2.00E−5 |
| 116 | >2.00E−5 |
| 117 | >2.00E−5 |
| 118 | 3.63E−06 |
| 119 | 6.21E−06 |
| 120 | 1.04E−05 |
| 121 | 1.01E−05 |
| 122 | 4.26E−06 |
| 123 | >2.00E−5 |
| 124 | 6.10E−06 |
| 125 | 3.79E−06 |
| 126 | >2.00E−5 |
| 127 | 6.32E−06 |
| 128 | >2.00E−5 |
| 129 | >2.00E−5 |
| 130 | 4.86E−06 |
| 131 | >2.00E−5 |
| 132 | >2.00E−5 |
| 133 | >2.00E−5 |
| 134 | >2.00E−5 |
| 135 | >2.00E−5 |
| 136 | 1.42E−06 |
| 137 | >2.00E−5 |
| 138 | >2.00E−5 |
| 139 | >2.00E−5 |
| 140 | >2.00E−5 |
| 141 | 1.29E−05 |
| 142 | >2.00E−5 |
| 143 | >2.00E−5 |
| 144 | >2.00E−5 |
| 145 | >2.00E−5 |
| 146 | >2.00E−5 |
| 147 | >2.00E−5 |
| 148 | >2.00E−5 |
| 149 | >2.00E−5 |
| 150 | >2.00E−5 |
| 151 | >2.00E−5 |
| 152 | 1.99E−06 |
| 153 | 1.23E−06 |
| 154 | 2.56E−06 |
| 155 | >2.00E−5 |
| 156 | >2.00E−5 |
| 157 | >2.00E−5 |
| 158 | >2.00E−5 |
| 159 | >2.00E−5 |
| 160 | >2.00E−5 |
| 161 | >2.00E−5 |
| 162 | 1.74E−05 |
| 163 | >2.00E−5 |
| 164 | >2.00E−5 |
| 165 | >2.00E−5 |
| 166 | >2.00E−5 |
| 167 | >2.00E−5 |
| 168 | >2.00E−5 |
| 169 | >2.00E−5 |
| 170 | >2.00E−5 |
| 171 | >2.00E−5 |
| 172 | >2.00E−5 |
| 173 | >2.00E−5 |
| 174 | >2.00E−5 |
| 175 | >2.00E−5 |
| 176 | >2.00E−5 |
| 177 | >2.00E−5 |
| 178 | >2.00E−5 |
| 179 | >2.00E−5 |
| 180 | >2.00E−5 |
| 181 | >2.00E−5 |
| 182 | >2.00E−5 |
| 183 | >2.00E−5 |
| 184 | 1.64E−06 |
| 185 | >2.00E−5 |
| 186 | >2.00E−5 |
| 187 | >2.00E−5 |
| 188 | >2.00E−5 |
| 189 | 2.20E−06 |
| 190 | >2.00E−5 |
| 191 | 7.34E−06 |
| 192 | >2.00E−5 |
| 193 | >2.00E−5 |
| 194 | >2.00E−5 |
| 195 | >2.00E−5 |
| 196 | >2.00E−5 |
| 197 | >2.00E−5 |
| 198 | >2.00E−5 |
| 199 | >2.00E−5 |
| 200 | >2.00E−5 |
| 201 | >2.00E−5 |
| 202 | >2.00E−5 |
| 203 | >2.00E−5 |
| 204 | >2.00E−5 |
| 205 | >2.00E−5 |
| 206 | 7.88E−06 |
| 207 | >2.00E−5 |
| 208 | >2.00E−5 |
| 209 | >2.00E−5 |
| 210 | >2.00E−5 |
| 211 | >2.00E−5 |
| 212 | >2.00E−5 |
| 213 | >2.00E−5 |
| 214 | >2.00E−5 |
| 215 | >2.00E−5 |
| 216 | >2.00E−5 |
| 217 | >2.00E−5 |
| 218 | >2.00E−5 |
| 219 | >2.00E−5 |
| 220 | >2.00E−5 |
| 221 | >2.00E−5 |
| 222 | >2.00E−5 |
| 223 | >2.00E−5 |
| 224 | >2.00E−5 |
| 225 | >2.00E−5 |
| 226 | >2.00E−5 |
| 227 | >2.00E−5 |
| 228 | >2.00E−5 |
| 229 | >2.00E−5 |
| 230 | >2.00E−5 |
| 231 | >2.00E−5 |
| 232 | >2.00E−5 |
| 233 | >2.00E−5 |
| 234 | >2.00E−5 |
| 235 | >2.00E−5 |
| 236 | >2.00E−5 |
| 237 | >2.00E−5 |
| 238 | >1.87E−5 |
| 239 | >2.00E−5 |
| 240 | >2.00E−5 |
| 241 | >2.00E−5 |
| 242 | >2.00E−5 |
| 243 | >2.00E−5 |
| 244 | >2.00E−5 |
| 245 | >2.00E−5 |
| 246 | >2.00E−5 |
| 247 | >2.00E−5 |
| 248 | >2.00E−5 |
| 249 | >2.00E−5 |
| 250 | >2.00E−5 |
| 251 | >2.00E−5 |
| 252 | >2.00E−5 |
| 253 | >2.00E−5 |
| 254 | >2.00E−5 |
| 255 | >2.00E−5 |
| 256 | >2.00E−5 |
| 257 | >2.00E−5 |
| 258 | >2.00E−5 |
| 259 | 7.37E−06 |
| 260 | >2.00E−5 |
| 261 | >2.00E−5 |
| 262 | >2.00E−5 |
| 263 | >2.00E−5 |
| 264 | >2.00E−5 |
| 265 | >2.00E−5 |
| 266 | >2.00E−5 |
| 267 | >2.00E−5 |
| 268 | >2.00E−5 |
| 269 | >2.00E−5 |
| 270 | >2.00E−5 |
| 271 | >2.00E−5 |
| 272 | >2.00E−5 |
| 273 | >2.00E−5 |
| 274 | >2.00E−5 |
| 275 | >2.00E−5 |
| 276 | >2.00E−5 |
| 277 | >2.00E−5 |
| 278 | >2.00E−5 |
| 279 | >2.00E−5 |
| 280 | >2.00E−5 |
| 281 | >2.00E−5 |
| 282 | >2.00E−5 |
| 283 | >2.00E−5 |
| 284 | >2.00E−5 |
| 285 | >2.00E−5 |
| 286 | >2.00E−5 |
| 287 | >2.00E−5 |
| 288 | >2.00E−5 |
| 289 | >2.00E−5 |
| 290 | >2.00E−5 |
| 291 | >2.00E−5 |
| 292 | >6.25E−6 |
| 293 | >2.00E−5 |
| 294 | 1.53E−05 |
| 295 | 9.27E−06 |
| 296 | >2.00E−5 |
| 297 | >2.00E−5 |
| 298 | >2.00E−5 |
| 299 | >2.00E−5 |
| 300 | >2.00E−5 |
| 301 | >2.00E−5 |
| 302 | >2.00E−5 |
| 303 | >2.00E−5 |
| 304 | >2.00E−5 |
| 305 | >2.00E−5 |
| 306 | >2.00E−5 |
| 307 | >2.00E−5 |
| 308 | >2.00E−5 |
| 309 | >2.00E−5 |
| 310 | 8.49E−6 |
| 311 | >2.00E−5 |
| 312 | >2.00E−5 |
| 313 | >2.00E−5 |
| 314 | >2.00E−5 |
| 315 | >2.00E−5 |
| 316 | 1.29E−5 |
| 317 | >2.00E−5 |
| 318 | >2.00E−5 |
| 319 | >2.00E−5 |
| 320 | >2.00E−5 |
| 321 | 7.30E−6 |
| 322 | >2.00E−5 |
| 323 | >2.00E−5 |
| 324 | 1.23E−5 |
| 325 | >2.00E−5 |
| 326 | 1.70E−5 |
| 327 | >2.00E−5 |
| 328 | 1.50E−5 |
| 329 | 1.38E−5 |
| 330 | >2.00E−5 |
| 331 | 4.90E−6 |
| 332 | >2.00E−5 |
| 333 | >2.00E−5 |
| 334 | 8.14E−6 |
| 335 | >2.00E−5 |
| 336 | 1.08E−5 |
| 337 | >2.00E−5 |
| 338 | 8.30E−6 |
| 339 | 1.38E−5 |
| 340 | 1.26E−5 |
| 341 | 1.74E−5 |
| 342 | >2.00E−5 |
| 343 | 4.27E−6 |
| 344 | >2.00E−5 |
| 345 | 6.24E−6 |
| 346 | >2.00E−5 |
| 347 | 1.04E−5 |
| 348 | 1.40E−5 |
| 349 | >2.00E−5 |
| 350 | >2.00E−5 |
| 351 | >2.00E−5 |
| 352 | 1.68E−5 |
| 353 | 1.27E−5 |
| 354 | >2.00E−5 |
| 355 | >2.00E−5 |
| 356 | >2.00E−5 |
| 357 | 1.00E−5 |
| 358 | >2.00E−5 |
| 359 | 7.13E−6 |
| 360 | 1.08E−5 |
| 361 | 1.38E−5 |
| 362 | >2.00E−5 |
| 363 | >2.00E−5 |
| 364 | >2.00E−5 |
| 365 | >2.00E−5 |
| 366 | >2.00E−5 |
| 367 | 1.76E−5 |
| 368 | >2.00E−5 |
| 369 | >2.00E−5 |
| 370 | >2.00E−5 |
| 371 | >2.00E−5 |
| 372 | 3.93E−6 |
| 373 | 1.44E−5 |
| 374 | 1.64E−6 |
| 375 | 1.45E−5 |
| 376 | >2.00E−5 |
| 377 | >2.00E−5 |
| 378 | 1.27E−5 |
| 379 | 1.13E−5 |
| 380 | 5.93E−6 |
| 381 | 1.40E−5 |
| 382 | >2.00E−5 |
| 383 | >2.00E−5 |
| 384 | >2.00E−5 |
| 385 | 1.29E−6 |
| 386 | 7.49E−7 |
| 387 | 5.93E−6 |
| 388 | >2.00E−5 |
| 389 | >2.00E−5 |
| 390 | >2.00E−5 |
| 391 | >2.00E−5 |
| 392 | >2.00E−5 |
| 393 | >2.00E−5 |
| 394 | >2.00E−5 |
| 395 | >2.00E−5 |
| 396 | >2.00E−5 |
| 397 | >2.00E−5 |
| 398 | >2.00E−5 |
| 399 | >2.00E−5 |
| 400 | >2.00E−5 |
| 401 | >2.00E−5 |
| 402 | 5.40E−6 |
| 403 | >2.00E−5 |
| 404 | >2.00E−5 |
| 405 | >2.00E−5 |
| 406 | >2.00E−5 |
| 407 | >2.00E−5 |
| 408 | >2.00E−5 |
| 409 | >2.00E−5 |
| 410 | >2.00E−5 |
| 411 | >2.00E−5 |
| 412 | >2.00E−5 |
| 413 | >2.00E−5 |
| 414 | >2.00E−5 |
| 415 | >2.00E−5 |
| 416 | >2.00E−5 |
| 417 | >1.63E−6 |
| 418 | >2.00E−5 |
| 419 | >2.00E−5 |
| 420 | >2.00E−5 |
| 421 | >2.00E−5 |
| 422 | >2.00E−5 |
| 423 | >2.00E−5 |
| 424 | >2.00E−5 |
| 425 | >2.00E−5 |
| 426 | 1.27E−5 |
| 427 | 4.23E−6 |
| 428 | >2.00E−5 |
| 429 | >2.00E−5 |
| 430 | >2.00E−5 |
| 431 | >2.00E−5 |
| 432 | 1.57E−5 |
| 433 | >2.00E−5 |
| 434 | >2.00E−5 |
| 435 | >2.00E−5 |
| 436 | >2.00E−5 |
| 437 | >2.00E−5 |
| 438 | >2.00E−5 |
| 439 | >2.00E−5 |
| 440 | >2.00E−5 |
| 441 | >2.00E−5 |
| 442 | >2.00E−5 |
| 443 | >2.00E−5 |
| 444 | >2.00E−5 |

3. qRT-PCR Assay: BRCA, ATR, MCL1 in MDA-MB-231, CAL-120

Tissue cultured human MDA-MB-231 human breast cancer cells were plated in 500 microL per well at 200,000 cells/well in a 24 well microtiter plate. After 24 h, the cells were exposed continuously for 24 h to test substances (substances were added with Tecan HP 0300 Dispenser). RNA was prepared using Qiagen RNeasy MiniKit (#74106), RNA was quantified using a NanoDrop Equipment, and 600 nano gamms of RNA was coverted to cDNA using a SuperScript VILO kit (Thermofisher #11755050) followed by qPCR amplification. BRCA1 and ATR gene expression was measured by RT-qPCR and normalised to GAPDH housekeeping gene expression. qPCR primer sets have been purchased from Thermo Fisher Scientific/Applied Biosystems: BRCA1, #Hs 01556193; ATR, #Hs 00992123; GAPDH, #Hs 03929097.

TABLE 4
Inhibition of BRCA1 mRNA
expression in MDA-MB-231 and CAL-120 cells

| | RTqPCR-MDA-MB-231, | RTqPCR-CAL-120, |
|---|---|---|
| | IC50 [mol/l], BRCA1 | IC50 [mol/l], BRCA1 |
| Example No | (median) | (median) |
| 1 | 1.66E−8 | |
| 2 | 2.08E−8 | |
| 3 | | |
| 4 | 7.13E−9 | 7.06E−9 |
| 5 | 2.33E−8 | |
| 6 | 5.43E−9 | |
| 7 | 2.03E−7 | |
| 8 | 9.37E−8 | |
| 9 | 2.55E−7 | |
| 10 | 2.53E−8 | |
| 11 | 3.18E−8 | |
| 12 | 1.17E−7 | |
| 13 | | |
| 14 | <3.00E−8 | |
| 15 | 1.00E−8 | 2.98E−8 |
| 16 | 5.41E−9 | |
| 17 | 1.20E−8 | |
| 18 | 1.14E−8 | |
| 19 | | |
| 20 | 6.81E−9 | 1.77E−8 |
| 21 | 3.18E−8 | 2.11E−7 |
| 22 | 1.31E−7 | |
| 23 | 3.83E−9 | |
| 24 | 1.00E−8 | 3.45E−8 |
| 25 | 3.01E−9 | |
| 26 | 7.44E−8 | |
| 27 | 1.65E−7 | |
| 28 | 1.33E−8 | 3.06E−8 |
| 29 | 2.12E−8 | 8.41E−8 |
| 30 | 2.85E−8 | |
| 31 | 1.50E−8 | 5.49E−8 |
| 32 | | |
| 33 | 2.37E−8 | |
| 34 | 1.00E−8 | |
| 35 | 3.90E−8 | |
| 36 | 1.28E−7 | |
| 37 | | |
| 38 | | |
| 39 | 9.23E−8 | |
| 40 | 1.46E−8 | |
| 41 | 3.67E−6 | |
| 42 | 3.40E−9 | |
| 43 | 6.99E−8 | |
| 44 | 2.95E−9 | |
| 45 | 9.90E−8 | |
| 46 | 1.25E−8 | |
| 47 | 2.32E−8 | |
| 48 | 9.42E−8 | |
| 49 | 6.92E−8 | |
| 50 | 1.82E−8 | |
| 51 | 5.80E−9 | |
| 52 | 2.60E−8 | |
| 53 | 7.71E−8 | |
| 54 | 6.62E−8 | |
| 55 | 4.76E−9 | |
| 56 | 2.18E−8 | |
| 57 | 1.52E−9 | |
| 58 | 4.90E−8 | |
| 59 | 3.05E−8 | |
| 60 | 4.53E−8 | |
| 61 | 9.54E−9 | |
| 62 | 3.77E−8 | |
| 63 | 3.41E−7 | |
| 64 | 2.79E−8 | |
| 65 | 1.44E−8 | |
| 66 | 6.24E−7 | |
| 67 | 2.48E−8 | |
| 68 | 4.34E−8 | |
| 69 | | |
| 70 | 5.59E−8 | |
| 71 | 2.11E−8 | |
| 72 | 3.86E−8 | |
| 73 | 4.95E−8 | |
| 74 | | |
| 75 | | |
| 76 | | |
| 77 | | |
| 78 | | |
| 79 | | |
| 80 | 1.83E−8 | |
| 81 | | |
| 82 | | |
| 83 | | |
| 84 | 1.18E−7 | |
| 85 | 1.83E−8 | |
| 86 | 1.71E−8 | |
| 87 | 1.07E−8 | |
| 88 | | |
| 89 | | |
| 90 | 1.37E−8 | |
| 91 | 8.55E−8 | |
| 92 | 1.33E−8 | |
| 93 | | |
| 94 | 2.22E−7 | |
| 95 | 1.47E−7 | |
| 96 | 2.94E−8 | |
| 97 | 1.68E−9 | |
| 98 | 7.88E−8 | |
| 99 | 1.12E−8 | |
| 100 | 6.22E−8 | |
| 101 | 3.14E−9 | |
| 102 | 2.96E−8 | |
| 103 | 7.19E−8 | |
| 104 | 3.00E−8 | |
| 105 | 8.43E−9 | |
| 106 | 3.22E−8 | |
| 107 | 1.51E−7 | |
| 108 | 8.74E−8 | |
| 109 | 1.34E−9 | |
| 110 | 7.51E−9 | |
| 111 | 1.53E−8 | |
| 112 | 2.37E−08 | |
| 113 | 2.11E−08 | |
| 114 | | |
| 115 | | |
| 116 | 1.23E−07 | |
| 117 | 1.21E−07 | |
| 118 | 2.61E−08 | |
| 119 | 2.13E−08 | |
| 120 | 3.00E−08 | |
| 121 | 3.44E−07 | |
| 122 | 2.46E−08 | |
| 123 | 7.12E−08 | |
| 124 | 4.66E−09 | |
| 125 | 6.11E−08 | |
| 126 | 1.01E−08 | |
| 127 | 1.62E−08 | |
| 128 | 1.46E−08 | |
| 129 | 1.74E−07 | |
| 130 | 3.76E−08 | |
| 131 | | |
| 132 | 2.95E−08 | |
| 133 | 4.23E−09 | |
| 134 | 5.50E−09 | |
| 135 | 3.00E−08 | |
| 136 | 3.38E−08 | |
| 137 | 2.05E−07 | |
| 138 | | |
| 139 | | |
| 140 | | |
| 141 | 1.64E−08 | |
| 142 | 1.91E−08 | |
| 143 | | |
| 144 | 8.33E−08 | |
| 145 | 1.90E−07 | |
| 146 | 1.61E−08 | |
| 147 | 7.67E−08 | |
| 148 | 8.76E−08 | |
| 149 | 1.48E−07 | |
| 150 | 1.30E−08 | |
| 151 | 1.83E−08 | |
| 152 | 4.25E−09 | |
| 153 | 8.20E−09 | |
| 154 | 7.04E−09 | |
| 155 | | |
| 156 | | |
| 157 | 9.03E−10 | |
| 158 | 8.83E−10 | |
| 159 | 1.68E−09 | |
| 160 | 2.95E−09 | |
| 161 | 2.53E−09 | |
| 162 | 1.09E−09 | |
| 163 | 1.03E−09 | |
| 164 | 2.55E−09 | |
| 165 | 2.35E−09 | |
| 166 | 4.62E−08 | |
| 167 | 4.29E−07 | |
| 168 | 2.46E−09 | |
| 169 | 3.32E−09 | |
| 170 | 4.19E−10 | |
| 171 | 1.03E−09 | |
| 172 | 6.26E−10 | |
| 173 | 4.58E−09 | |
| 174 | 8.72E−10 | |
| 175 | 1.63E−09 | |
| 176 | 4.68E−09 | |
| 177 | | |
| 178 | | |
| 179 | 2.17E−08 | |
| 180 | 1.20E−08 | |
| 181 | 3.51E−09 | |
| 182 | 3.32E−09 | |
| 183 | 4.17E−09 | |
| 184 | 3.89E−09 | |
| 185 | 2.66E−08 | |
| 186 | 1.49E−09 | |
| 187 | 9.17E−09 | |
| 188 | 3.06E−09 | |
| 189 | 1.98E−09 | |
| 190 | 3.27E−09 | |
| 191 | 2.18E−09 | |
| 192 | 2.05E−09 | |
| 193 | 3.65E−09 | |
| 194 | 8.29E−10 | |
| 195 | 2.83E−09 | |
| 196 | 4.27E−09 | |
| 197 | 6.27E−09 | |
| 198 | 6.03E−08 | |
| 199 | 4.79E−08 | |
| 200 | 6.62E−08 | |
| 201 | 1.45E−07 | |
| 202 | | |
| 203 | 1.19E−09 | |
| 204 | 1.13E−09 | |
| 205 | 1.81E−09 | |
| 206 | 7.63E−09 | |
| 207 | 3.50E−09 | |
| 208 | 6.10E−09 | |
| 209 | 1.67E−09 | |
| 210 | 2.27E−09 | |
| 211 | 4.24E−09 | |
| 212 | 5.49E−09 | |
| 213 | 1.50E−08 | |
| 214 | 1.93E−08 | |
| 215 | 1.31E−07 | |
| 216 | 7.36E−09 | |
| 217 | 3.35E−08 | |
| 218 | 1.23E−08 | |
| 219 | 1.16E−08 | |
| 220 | 4.18E−09 | |
| 221 | 1.18E−08 | |
| 222 | | |
| 223 | 8.65E−08 | |
| 224 | 2.12E−08 | |
| 225 | 3.48E−08 | |
| 226 | | |
| 227 | 1.53E−08 | |
| 228 | | |
| 229 | 1.08E−08 | |
| 230 | | |
| 231 | | |
| 232 | 4.92E−09 | |
| 233 | | |
| 234 | 5.76E−09 | |
| 235 | | |
| 236 | 2.04E−08 | |
| 237 | | |
| 238 | >1.24E−8 | |
| 239 | | |
| 240 | 7.69E−09 | |
| 241 | 1.73E−08 | |
| 242 | 5.14E−07 | |
| 243 | | |
| 244 | | |
| 245 | 7.35E−09 | |
| 246 | 1.03E−08 | |
| 247 | 5.55E−09 | |
| 248 | 1.60E−09 | |
| 249 | 5.69E−09 | |
| 250 | 1.05E−08 | |
| 251 | 1.49E−09 | |
| 252 | 1.02E−08 | |
| 253 | 2.08E−08 | |
| 254 | 6.05E−08 | |
| 255 | | |
| 256 | 6.46E−09 | |
| 257 | 6.97E−09 | |
| 258 | 1.21E−08 | |
| 259 | 1.14E−08 | |
| 260 | 3.42E−09 | |
| 261 | | |
| 262 | | |
| 263 | 2.88E−08 | |
| 264 | 6.32E−09 | |
| 265 | | |
| 266 | | |
| 267 | | |
| 268 | 3.23E−09 | |
| 269 | 8.39E−09 | |
| 270 | 6.41E−09 | |
| 271 | 1.39E−08 | |
| 272 | 7.91E−09 | |
| 273 | 1.95E−09 | |
| 274 | 1.92E−09 | |
| 275 | 2.01E−09 | |
| 276 | | |
| 277 | 5.42E−09 | |
| 278 | 3.89E−09 | |
| 279 | 1.20E−08 | |
| 280 | | |
| 281 | | |
| 282 | | |
| 283 | 9.13E−09 | |
| 284 | 2.42E−08 | |
| 285 | | |
| 286 | 1.06E−08 | |
| 287 | 2.08E−08 | |
| 288 | 2.69E−09 | |
| 289 | 2.91E−09 | |
| 290 | 1.76E−08 | |
| 291 | 1.53E−08 | |
| 292 | 2.80E−09 | |
| 293 | 5.68E−09 | |
| 294 | 1.16E−09 | |
| 295 | 4.81E−09 | |
| 296 | 1.27E−09 | |
| 297 | 5.15E−08 | |
| 298 | 4.12E−09 | |
| 299 | 8.86E−09 | |
| 300 | 1.26E−08 | |
| 301 | 6.73E−09 | |
| 302 | 9.06E−09 | |
| 303 | 4.79E−09 | |
| 304 | | |
| 305 | 6.23E−09 | |
| 306 | 6.94E−09 | |
| 307 | 8.07E−09 | |
| 308 | 5.46E−09 | |
| 309 | 5.83E−09 | |
| 310 | 3.51E−08 | |
| 311 | 3.42E−09 | |
| 312 | 2.04E−09 | |
| 313 | 2.21E−08 | |
| 314 | 3.57E−08 | |
| 315 | 7.53E−08 | |
| 316 | 1.24E−08 | |
| 317 | 6.90E−09 | |
| 318 | 9.70E−10 | |
| 319 | 9.97E−09 | |
| 320 | 3.59E−09 | |
| 321 | 1.30E−09 | |
| 322 | !3.56E−9 | |
| 323 | 1.69E−08 | |
| 324 | 8.86E−09 | |
| 325 | 3.91E−09 | |
| 326 | 1.63E−09 | |
| 327 | 8.30E−09 | |
| 328 | 2.78E−09 | |
| 329 | 9.23E−09 | |
| 330 | 5.68E−09 | |
| 331 | 2.56E−09 | |
| 332 | 3.78E−09 | |
| 333 | 1.80E−09 | |
| 334 | 4.02E−09 | |
| 335 | 1.27E−08 | |
| 336 | 8.00E−09 | |
| 337 | <1.00E−8 | |
| 338 | 1.30E−09 | |
| 339 | 7.85E−09 | |
| 340 | 8.16E−09 | |
| 341 | 5.13E−09 | |
| 342 | 1.27E−09 | |
| 343 | 1.91E−09 | |
| 344 | 2.34E−09 | |
| 345 | 2.83E−09 | |
| 346 | 3.02E−09 | |
| 347 | 2.04E−09 | |
| 348 | 1.97E−09 | |
| 349 | 1.56E−08 | |
| 350 | 1.37E−08 | |
| 351 | 6.16E−08 | |
| 352 | 4.93E−09 | |
| 353 | 2.39E−09 | |
| 354 | <1.00E−7 | |
| 355 | 1.41E−09 | |
| 356 | 5.05E−09 | |
| 357 | 6.05E−09 | |
| 358 | 3.87E−09 | |
| 359 | 3.28E−09 | |
| 360 | 1.03E−09 | |
| 361 | 9.02E−09 | |
| 362 | 2.64E−08 | |
| 363 | 2.25E−08 | |
| 364 | 9.75E−09 | |
| 365 | | |
| 366 | 2.71E−08 | |
| 367 | | |
| 368 | | |
| 369 | | |
| 370 | 5.63E−09 | |
| 371 | 7.36E−09 | |
| 372 | 2.16E−09 | |
| 373 | 1.27E−09 | |
| 374 | 2.60E−09 | |
| 375 | | |
| 376 | | |
| 377 | | |
| 378 | | |
| 379 | 2.36E−09 | |
| 380 | 1.00E−09 | |
| 381 | | |
| 382 | 2.88E−08 | |
| 383 | 4.01E−09 | |
| 384 | 1.36E−08 | |
| 385 | 3.16E−08 | |
| 386 | 9.16E−09 | |
| 387 | 5.56E−09 | |
| 388 | 4.00E−08 | |
| 389 | | |
| 390 | | |
| 391 | 3.77E−09 | |
| 392 | 2.55E−09 | |
| 393 | | |
| 394 | 1.14E−08 | |
| 395 | 1.47E−08 | |
| 396 | | |
| 397 | | |
| 398 | | |
| 399 | | |
| 400 | 1.40E−08 | |
| 401 | | |
| 402 | 3.81E−09 | |
| 403 | | |
| 404 | | |
| 405 | | |
| 406 | | |
| 407 | 1.60E−09 | |
| 408 | | |
| 409 | | |
| 410 | 3.42E−09 | |
| 411 | | |
| 412 | | |
| 413 | 8.27E−08 | |
| 414 | | |
| 415 | | |
| 416 | | |
| 417 | 1.46E−08 | |
| 418 | | |
| 419 | | |
| 420 | | |
| 421 | 7.46E−08 | |
| 422 | 7.43E−09 | |
| 423 | 6.20E−09 | |
| 424 | | |
| 425 | 2.68E−09 | |
| 426 | 3.21E−09 | |
| 427 | 4.50E−08 | |
| 428 | | |
| 429 | 7.47E−09 | |
| 430 | 1.00E−08 | |
| 431 | 4.17E−09 | |
| 432 | 1.76E−09 | |
| 433 | 1.06E−08 | |
| 434 | 1.05E−08 | |
| 435 | 9.59E−10 | |
| 436 | | |
| 437 | 6.19E−09 | |
| 438 | | |
| 439 | | |
| 440 | 2.70E−09 | |
| 441 | 3.39E−09 | |
| 442 | 7.38E−10 | |
| 443 | 2.21E−09 | |
| 444 | 5.15E−08 | |

### 4. Proliferation Assay: MDA-MB-231, CAL-120

Human tumour cells were originally obtained from the American Type Culture Collection (ATCC), or from the Deutsche Sammlung von Mikroorganismen und Zellkulturen (DSMZ, German Collection of Microorganisms and Cell Cultures). Cultivated tumour cells (CAL-120, human breast adenocarcinoma cells, DSMZ ACC-459; MDA-MB-231, human breast cancer cells, ATCC HTB-26) were plated at a density of 4,000 cells/well in a 96-well multititer plate in 200 microL of their respective growth medium supplemented 10% fetal calf serum. After 24 hours, the cells of one plate (zero-point plate) were stained with crystal violet (see below), while the medium of the other plates was supplemented with the test substances in various concentrations (0 micromol/L, as well as in the range of 0.01-10 micromol/L; the final concentration of the solvent dimethyl sulfoxide was adjusted to 0.1%) using a Tecan HP D300 Digital Dispenser. The cells were incubated for 4 days in the presence of test substances. Cell proliferation was determined by staining the cells with crystal violet: the cells were fixed by adding 20 microL/measuring point of an 11% glutaric aldehyde solution for 15 minutes at room temperature. After three washing cycles of the fixed cells with water, the plates were dried at room temperature. The cells were stained by adding 100 microL/measuring point of a 0.1% crystal violet solution (pH 3.0). After three washing cycles of the stained cells with water, the plates were dried at room temperature. The dye was dissolved by adding 100 microL/measuring point of a 10% acetic acid solution. The extinction was determined by photometry at a wavelength of 595 nm. The change of cell number, in percent, was calculated by normalization of the measured values to the extinction values of the zero-point plate (=0%) and the extinction of the untreated (0 μM) cells (=100%). The IC50 values (inhibitory concentration at 50% of maximal effect) were determined by means of a 4 parameter fit.

TABLE 5
Antiproliferative data for the compounds of the present invention.

| | Prolif-MDA MB- | Prolif-CAL- |
|---|---|---|
| Example | 231, IC50- | 120, IC50- |
| No | [mol/l] (median) | [mol/l] (median) |
| 1 | 8.64E−8 | |
| 2 | | 3.12E−8 |
| 3 | | |
| 4 | 2.53E−8 | 1.20E−8 |
| 5 | | |
| 6 | 1.61E−8 | |
| 7 | | |
| 8 | | |
| 9 | 1.18E−6 | |
| 10 | 7.99E−8 | |
| 11 | | |
| 12 | 3.73E−7 | |
| 13 | | |
| 14 | 4.70E−8 | |
| 15 | 4.46E−8 | 2.95E−8 |
| 16 | 8.11E−8 | 3.82E−8 |
| 17 | 1.07E−7 | |
| 18 | 3.10E−8 | 3.24E−8 |
| 19 | | |
| 20 | 3.10E−8 | 1.42E−8 |
| 21 | 2.85E−7 | 1.61E−7 |
| 22 | 5.93E−7 | |
| 23 | 3.39E−8 | |
| 24 | 5.33E−8 | 3.57E−8 |
| 25 | | 2.62E−9 |
| 26 | | |
| 27 | | |
| 28 | 7.45E−8 | 3.91E−8 |
| 29 | 8.99E−8 | 1.70E−7 |
| 30 | | |
| 31 | 4.98E−8 | 2.10E−8 |
| 32 | | |
| 33 | 2.90E−7 | |
| 34 | 4.86E−8 | |
| 35 | | |
| 36 | | |
| 37 | | |
| 38 | | |
| 39 | | |
| 40 | | 2.31E−8 |
| 41 | | 1.18E−5 |
| 42 | 5.74E−9 | 1.76E−9 |
| 43 | | 1.31E−7 |
| 44 | | 1.46E−9 |
| 45 | | 8.91E−8 |
| 46 | | 2.38E−7 |
| 47 | | 2.39E−8 |
| 48 | | 1.20E−7 |
| 49 | | 4.40E−8 |
| 50 | | 2.95E−8 |
| 51 | | 9.43E−9 |
| 52 | | 2.33E−8 |
| 53 | | 1.06E−7 |
| 54 | | 5.38E−8 |
| 55 | | 6.28E−9 |
| 56 | | 1.73E−8 |
| 57 | | 3.14E−9 |
| 58 | | 5.63E−8 |
| 59 | | 1.25E−8 |
| 60 | | 2.57E−8 |
| 61 | 3.08E−8 | 2.43E−8 |
| 62 | | 3.25E−8 |
| 63 | | 3.60E−8 |
| 64 | | 3.61E−8 |
| 65 | | 1.00E−8 |
| 66 | | 2.30E−7 |
| 67 | | 2.08E−8 |
| 68 | | 4.02E−8 |
| 69 | | 3.33E−7 |
| 70 | | 4.90E−8 |
| 71 | | 3.20E−8 |
| 72 | | 3.24E−8 |
| 73 | | 2.30E−8 |
| 74 | | 3.11E−7 |
| 75 | | 5.32E−8 |
| 76 | | 1.10E−7 |
| 77 | | 3.38E−7 |
| 78 | | 2.57E−8 |
| 79 | | 4.67E−9 |
| 80 | | 1.42E−8 |
| 81 | | 4.14E−9 |
| 82 | | 8.52E−8 |
| 83 | | 1.21E−8 |
| 84 | | 1.07E−7 |
| 85 | | 2.63E−8 |
| 86 | | 1.44E−8 |
| 87 | | 6.58E−9 |
| 88 | | 1.42E−8 |
| 89 | | 3.29E−8 |
| 90 | | 1.74E−8 |
| 91 | | 1.05E−7 |
| 92 | | 1.20E−8 |
| 93 | | 1.00E−7 |
| 94 | | 1.77E−7 |
| 95 | | 2.30E−7 |
| 96 | | 3.01E−8 |
| 97 | | 5.17E−9 |
| 98 | | 1.16E−7 |
| 99 | | 5.47E−8 |
| 100 | | 6.02E−8 |
| 101 | | 4.10E−9 |
| 102 | | 2.70E−8 |
| 103 | | 4.65E−8 |
| 104 | | 1.27E−7 |
| 105 | | 7.36E−9 |
| 106 | | 4.70E−8 |
| 107 | | 1.88E−7 |
| 108 | | 8.96E−8 |
| 109 | | 4.90E−9 |
| 110 | | 5.03E−9 |
| 111 | | 8.96E−9 |
| 112 | 1.86E−07 | |
| 113 | | |
| 114 | | |
| 115 | | |
| 116 | | 4.70E−08 |
| 117 | | 2.31E−08 |
| 118 | | 2.97E−08 |
| 119 | | 7.75E−09 |
| 120 | | 2.54E−08 |
| 121 | | 4.04E−07 |
| 122 | | 1.85E−08 |
| 123 | | 8.32E−08 |
| 124 | | 6.43E−09 |
| 125 | | 5.31E−08 |
| 126 | 1.31E−08 | 1.10E−08 |
| 127 | 3.25E−08 | 2.82E−08 |
| 128 | 1.60E−08 | 1.27E−08 |
| 129 | 1.32E−07 | 1.25E−07 |
| 130 | 3.56E−08 | 3.48E−08 |
| 131 | 6.82E−07 | 5.95E−07 |
| 132 | 4.30E−08 | 4.34E−08 |
| 133 | 4.94E−09 | 4.41E−09 |
| 134 | 1.17E−08 | 1.16E−08 |
| 135 | 4.40E−08 | 4.17E−08 |
| 136 | 2.64E−08 | 1.71E−08 |
| 137 | 1.92E−07 | 1.10E−07 |
| 138 | 5.56E−07 | 4.51E−07 |
| 139 | 5.39E−07 | 4.46E−07 |
| 140 | 3.15E−07 | 2.55E−07 |
| 141 | 3.78E−08 | 2.45E−08 |
| 142 | 2.37E−08 | 1.13E−08 |
| 143 | 8.27E−07 | 3.06E−07 |
| 144 | 1.85E−07 | 1.10E−07 |
| 145 | 1.62E−06 | 2.23E−07 |
| 146 | 4.33E−08 | 3.10E−08 |
| 147 | 3.17E−07 | 3.10E−07 |
| 148 | | 1.09E−07 |
| 149 | 2.76E−07 | 1.64E−07 |
| 150 | 3.89E−08 | 1.83E−08 |
| 151 | 3.72E−08 | 2.47E−08 |
| 152 | 1.27E−08 | 3.86E−09 |
| 153 | 2.44E−08 | 5.78E−09 |
| 154 | 2.62E−08 | 7.69E−09 |
| 155 | 1.74E−07 | 7.53E−08 |
| 156 | 2.05E−08 | 5.45E−09 |
| 157 | 4.79E−09 | 1.78E−09 |
| 158 | 4.24E−09 | 1.17E−09 |
| 159 | 9.25E−09 | 1.73E−09 |
| 160 | 9.22E−09 | 6.50E−09 |
| 161 | 1.13E−08 | 3.30E−09 |
| 162 | 4.02E−09 | 1.55E−09 |
| 163 | 3.96E−09 | 1.05E−09 |
| 164 | 2.57E−08 | 4.13E−09 |
| 165 | 9.50E−08 | 4.16E−09 |
| 166 | 1.64E−07 | 2.01E−08 |
| 167 | 7.07E−07 | 7.04E−08 |
| 168 | | 3.25E−09 |
| 169 | 2.07E−08 | 3.56E−09 |
| 170 | 2.25E−09 | 1.00E−09 |
| 171 | 5.49E−09 | 2.66E−09 |
| 172 | 5.35E−09 | 2.43E−09 |
| 173 | 2.08E−08 | 8.14E−09 |
| 174 | 2.19E−09 | 1.00E−09 |
| 175 | 3.26E−09 | 1.15E−09 |
| 176 | 1.46E−08 | 4.37E−09 |
| 177 | 3.32E−07 | 6.88E−08 |
| 178 | 5.01E−07 | 9.68E−08 |
| 179 | 7.48E−08 | 1.67E−08 |
| 180 | 1.39E−08 | 1.27E−09 |
| 181 | 9.29E−09 | 6.10E−09 |
| 182 | 1.02E−08 | 3.70E−09 |
| 183 | 5.60E−08 | 2.74E−08 |
| 184 | 1.03E−08 | 9.27E−09 |
| 185 | 5.88E−08 | 1.86E−08 |
| 186 | 4.94E−09 | 4.20E−09 |
| 187 | 1.38E−08 | 1.10E−09 |
| 188 | 1.61E−08 | 1.76E−08 |
| 189 | 1.07E−08 | 4.67E−09 |
| 190 | 4.01E−09 | 6.68E−09 |
| 191 | 9.68E−09 | 1.16E−09 |
| 192 | 4.91E−09 | 3.08E−09 |
| 193 | 6.64E−09 | 5.06E−09 |
| 194 | 1.69E−09 | 1.16E−09 |
| 195 | 4.80E−09 | 4.46E−09 |
| 196 | 1.29E−08 | 8.05E−09 |
| 197 | 1.56E−08 | 4.99E−09 |
| 198 | 9.80E−08 | 3.11E−08 |
| 199 | 5.32E−08 | 1.54E−08 |
| 200 | 1.52E−07 | 9.58E−08 |
| 201 | 2.90E−07 | 1.26E−07 |
| 202 | 2.95E−07 | 1.31E−07 |
| 203 | 1.69E−09 | 1.02E−09 |
| 204 | 1.59E−09 | 8.69E−10 |
| 205 | 4.20E−09 | 1.73E−09 |
| 206 | 1.28E−08 | 7.28E−09 |
| 207 | 1.14E−08 | 4.41E−09 |
| 208 | 5.83E−09 | 1.79E−09 |
| 209 | 2.26E−09 | 1.28E−09 |
| 210 | 8.06E−09 | 4.66E−09 |
| 211 | 7.03E−09 | 2.29E−09 |
| 212 | 5.45E−09 | 2.29E−09 |
| 213 | 2.48E−08 | 1.07E−08 |
| 214 | 1.77E−08 | 6.20E−09 |
| 215 | 1.29E−07 | 3.43E−08 |
| 216 | 1.68E−08 | 8.55E−09 |
| 217 | 9.79E−08 | 4.49E−08 |
| 218 | 3.57E−08 | 2.11E−08 |
| 219 | 5.03E−08 | 2.72E−08 |
| 220 | 2.12E−08 | 7.04E−09 |
| 221 | 3.17E−08 | 1.30E−08 |
| 222 | 1.82E−07 | 1.07E−07 |
| 223 | 3.49E−07 | 2.70E−07 |
| 224 | 1.28E−07 | 5.70E−08 |
| 225 | 1.23E−07 | 6.27E−08 |
| 226 | 1.62E−07 | 1.16E−07 |
| 227 | 5.79E−08 | 4.42E−08 |
| 228 | 4.34E−07 | 2.73E−07 |
| 229 | 4.28E−08 | 3.94E−08 |
| 230 | 2.61E−07 | 3.13E−07 |
| 231 | 1.10E−07 | 1.35E−07 |
| 232 | 1.79E−08 | 5.25E−09 |
| 233 | 1.45E−07 | 1.58E−07 |
| 234 | 5.99E−08 | 3.13E−08 |
| 235 | 3.15E−07 | 2.74E−07 |
| 236 | 9.05E−08 | 9.74E−08 |
| 237 | 1.64E−07 | 1.64E−07 |
| 238 | 4.01E−08 | 3.63E−08 |
| 239 | 5.61E−07 | 4.36E−07 |
| 240 | 3.86E−08 | 2.37E−08 |
| 241 | 2.89E−08 | 2.96E−08 |
| 242 | 2.53E−07 | 8.57E−07 |
| 243 | | 3.67E−06 |
| 244 | 5.01E−07 | 2.18E−07 |
| 245 | 1.43E−08 | 3.10E−09 |
| 246 | 1.21E−07 | 1.21E−08 |
| 247 | 5.07E−08 | 9.79E−09 |
| 248 | 1.20E−08 | 3.13E−09 |
| 249 | 4.03E−08 | 4.28E−09 |
| 250 | 3.44E−08 | 3.49E−09 |
| 251 | 2.73E−09 | 6.60E−09 |
| 252 | 3.06E−08 | 3.87E−08 |
| 253 | 3.28E−08 | 6.91E−09 |
| 254 | 1.81E−07 | 8.77E−08 |
| 255 | 6.10E−08 | 3.28E−08 |
| 256 | 1.26E−08 | 9.97E−09 |
| 257 | 1.07E−08 | 1.08E−08 |
| 258 | 5.55E−08 | 2.79E−08 |
| 259 | 3.19E−08 | 1.19E−08 |
| 260 | 1.34E−08 | 6.03E−09 |
| 261 | 9.99E−08 | 3.89E−08 |
| 262 | 1.01E−07 | 6.09E−08 |
| 263 | 7.32E−08 | 2.89E−08 |
| 264 | 2.30E−08 | 8.70E−09 |
| 265 | 1.06E−07 | 3.54E−08 |
| 266 | 1.33E−07 | 1.02E−07 |
| 267 | 1.07E−07 | 6.94E−08 |
| 268 | 9.43E−09 | 5.59E−09 |
| 269 | 2.93E−08 | 7.31E−09 |
| 270 | 2.25E−08 | 6.92E−09 |
| 271 | 4.00E−08 | 2.32E−08 |
| 272 | 2.77E−08 | 2.24E−08 |
| 273 | 6.00E−09 | 4.49E−09 |
| 274 | 6.74E−09 | 4.89E−09 |
| 275 | 3.87E−09 | 3.88E−09 |
| 276 | 2.58E−07 | 1.65E−07 |
| 277 | 4.02E−08 | 3.09E−08 |
| 278 | 1.74E−08 | 7.54E−09 |
| 279 | 3.48E−08 | 1.72E−08 |
| 280 | 2.31E−07 | 6.41E−08 |
| 281 | 3.06E−07 | 8.54E−08 |
| 282 | 1.78E−07 | 4.52E−08 |
| 283 | 3.50E−08 | 2.61E−08 |
| 284 | 7.35E−08 | 7.38E−08 |
| 285 | 9.06E−08 | 8.03E−08 |
| 286 | 2.00E−08 | 2.08E−08 |
| 287 | 3.43E−08 | 3.32E−08 |
| 288 | 5.68E−09 | 1.80E−09 |
| 289 | 1.03E−08 | 3.08E−09 |
| 290 | 6.49E−08 | 2.03E−08 |
| 291 | 5.94E−08 | 1.86E−08 |
| 292 | 7.05E−09 | 3.01E−09 |
| 293 | 3.61E−08 | 8.88E−09 |
| 294 | 3.37E−09 | 1.42E−09 |
| 295 | 2.37E−08 | 7.25E−09 |
| 296 | 4.40E−09 | 1.52E−09 |
| 297 | 7.77E−08 | 2.23E−08 |
| 298 | 1.05E−08 | 4.05E−09 |
| 299 | 8.53E−09 | 4.71E−09 |
| 300 | 1.06E−07 | 1.62E−08 |
| 301 | 7.75E−09 | 5.74E−09 |
| 302 | 1.19E−08 | 8.98E−09 |
| 303 | 2.65E−08 | 7.90E−09 |
| 304 | 2.99E−07 | 8.67E−08 |
| 305 | 5.94E−09 | 4.44E−09 |
| 306 | 7.83E−09 | 7.21E−09 |
| 307 | 5.05E−09 | 4.60E−09 |
| 308 | 2.79E−08 | 1.02E−08 |
| 309 | 6.31E−09 | 4.11E−09 |
| 310 | 3.48E−09 | 1.74E−09 |
| 311 | 1.67E−08 | 8.13E−09 |
| 312 | 3.21E−09 | 2.32E−09 |
| 313 | 1.56E−08 | 9.52E−09 |
| 314 | 3.13E−08 | 1.30E−08 |
| 315 | 8.80E−08 | 3.84E−08 |
| 316 | 2.28E−08 | 7.16E−09 |
| 317 | 5.21E−09 | 3.66E−09 |
| 318 | 4.22E−09 | 2.80E−09 |
| 319 | 4.76E−09 | 2.87E−09 |
| 320 | 4.17E−09 | 4.24E−09 |
| 321 | 2.45E−09 | 2.35E−09 |
| 322 | 8.85E−09 | 7.97E−09 |
| 323 | 8.76E−09 | 5.40E−09 |
| 324 | 1.56E−09 | 1.17E−09 |
| 325 | 3.22E−09 | 1.48E−09 |
| 326 | 1.57E−09 | 1.25E−09 |
| 327 | 2.27E−09 | 2.28E−09 |
| 328 | 1.04E−09 | 9.01E−10 |
| 329 | 7.66E−09 | 6.11E−09 |
| 330 | 3.38E−09 | 2.44E−09 |
| 331 | 3.75E−09 | 3.08E−09 |
| 332 | 1.18E−08 | 1.09E−08 |
| 333 | 3.78E−09 | 3.10E−09 |
| 334 | 2.18E−09 | 1.72E−09 |
| 335 | 1.05E−08 | 4.24E−09 |
| 336 | 4.27E−09 | 3.21E−09 |
| 337 | 2.45E−09 | 2.04E−09 |
| 338 | 3.10E−09 | 1.93E−09 |
| 339 | 7.45E−09 | 4.79E−09 |
| 340 | 4.36E−09 | 3.36E−09 |
| 341 | 3.73E−09 | < 1.00E− |
| | | 10 |
| 342 | 1.97E−09 | 1.52E−09 |
| 343 | 2.84E−09 | 1.82E−09 |
| 344 | 4.82E−09 | 2.70E−09 |
| 345 | 3.44E−09 | 3.05E−09 |
| 346 | 4.67E−09 | 2.66E−09 |
| 347 | 4.35E−09 | 2.47E−09 |
| 348 | 2.00E−09 | 1.14E−09 |
| 349 | 1.29E−08 | 3.76E−09 |
| 350 | 1.37E−08 | 4.35E−09 |
| 351 | 9.39E−08 | 3.84E−08 |
| 352 | 1.88E−08 | 8.05E−09 |
| 353 | 1.81E−09 | 1.41E−09 |
| 354 | 3.05E−09 | 2.80E−09 |
| 355 | 3.20E−09 | 2.20E−09 |
| 356 | 5.25E−09 | 2.10E−09 |
| 357 | 1.04E−08 | 4.33E−09 |
| 358 | 1.48E−08 | 4.05E−09 |
| 359 | 1.52E−08 | 5.69E−09 |
| 360 | 5.67E−09 | 2.24E−09 |
| 361 | 2.32E−08 | 6.73E−09 |
| 362 | 3.35E−08 | 8.15E−09 |
| 363 | 3.46E−08 | 7.26E−09 |
| 364 | 4.13E−08 | |
| 365 | 1.18E−07 | 7.02E−08 |
| 366 | 5.71E−08 | 3.13E−08 |
| 367 | 8.41E−08 | 3.14E−08 |
| 368 | 1.73E−07 | 9.49E−08 |
| 369 | 2.97E−07 | 1.40E−07 |
| 370 | 7.18E−09 | 3.59E−09 |
| 371 | 7.80E−09 | 3.90E−09 |
| 372 | 4.28E−09 | 2.94E−09 |
| 373 | 3.33E−09 | 1.93E−09 |
| 374 | 3.62E−09 | 2.80E−09 |
| 375 | 1.42E−07 | 6.86E−08 |
| 376 | 1.02E−07 | 3.68E−08 |
| 377 | 1.16E−07 | 7.93E−08 |
| 378 | 9.64E−08 | 4.15E−08 |
| 379 | 2.92E−09 | 1.71E−09 |
| 380 | 2.19E−09 | 1.72E−09 |
| 381 | 4.35E−08 | 3.08E−08 |
| 382 | 2.93E−08 | 1.24E−08 |
| 383 | 3.00E−08 | 1.14E−08 |
| 384 | 8.48E−09 | 3.48E−09 |
| 385 | 4.38E−08 | 2.59E−08 |
| 386 | 2.02E−08 | 1.25E−08 |
| 387 | 4.57E−09 | 4.95E−09 |
| 388 | 3.02E−08 | 2.25E−08 |
| 389 | 3.32E−07 | 5.27E−08 |
| 390 | 1.76E−07 | 2.98E−08 |
| 391 | 2.61E−08 | 8.31E−09 |
| 392 | 1.11E−08 | 4.73E−09 |
| 393 | 1.20E−07 | 4.68E−08 |
| 394 | 1.98E−08 | 4.32E−09 |
| 395 | 3.40E−08 | 1.11E−08 |
| 396 | 1.57E−07 | 5.50E−08 |
| 397 | 3.99E−07 | 1.71E−07 |
| 398 | 5.92E−08 | 6.74E−08 |
| 399 | 3.84E−07 | 8.30E−08 |
| 400 | 2.03E−08 | 5.23E−09 |
| 401 | 1.32E−07 | 4.35E−08 |
| 402 | 8.56E−09 | 7.85E−09 |
| 403 | 5.24E−07 | 1.51E−07 |
| 404 | 1.84E−06 | 1.89E−07 |
| 405 | 4.39E−07 | 1.35E−07 |
| 406 | 2.83E−07 | 6.22E−08 |
| 407 | 2.28E−09 | 9.27E−10 |
| 408 | 7.41E−07 | 2.52E−07 |
| 409 | 3.43E−07 | 9.90E−08 |
| 410 | 8.27E−09 | 3.07E−09 |
| 411 | 1.38E−07 | 7.90E−08 |
| 412 | 1.91E−06 | 1.13E−06 |
| 413 | 2.98E−07 | 6.99E−08 |
| 414 | 6.87E−07 | 8.04E−08 |
| 415 | 5.84E−07 | 2.68E−07 |
| 416 | 9.86E−07 | 3.58E−07 |
| 417 | 8.31E−08 | 9.17E−09 |
| 418 | 2.46E−07 | 6.44E−08 |
| 419 | 2.28E−07 | 7.95E−08 |
| 420 | 5.25E−07 | 2.09E−07 |
| 421 | 8.75E−08 | 3.83E−08 |
| 422 | 2.01E−08 | 6.88E−09 |
| 423 | 1.82E−08 | 1.23E−08 |
| 424 | 7.37E−08 | 4.11E−08 |
| 425 | 8.76E−09 | 3.06E−09 |
| 426 | 4.19E−09 | 3.57E−09 |
| 427 | 1.13E−07 | 4.40E−08 |
| 428 | 1.74E−07 | 4.74E−08 |
| 429 | 3.24E−08 | 8.86E−09 |
| 430 | 8.15E−08 | 3.86E−08 |
| 431 | 2.84E−08 | 7.02E−09 |
| 432 | 8.53E−09 | 3.66E−09 |
| 433 | 4.03E−08 | 1.31E−08 |
| 434 | 4.47E−08 | 1.02E−08 |
| 435 | 4.25E−09 | 1.76E−09 |
| 436 | 4.16E−07 | 1.08E−07 |
| 437 | 4.58E−08 | 1.09E−08 |
| 438 | 2.83E−07 | 4.46E−08 |
| 439 | 1.80E−07 | 3.63E−08 |
| 440 | 2.14E−08 | 7.39E−09 |
| 441 | 2.85E−08 | 7.69E−09 |
| 442 | 6.38E−09 | 2.61E−09 |
| 443 | 6.17E−09 | 5.31E−09 |
| 444 | 6.91E−08 | 1.49E−08 |

### 5. Proliferation Assay: Cell Line Panel

Human tumour cells were originally obtained from the American Type Culture Collection (ATCC), from Cell Line Services GmbH (CLS), from the Deutsche Sammlung von Mikroorganismen und Zellkulturen (DSMZ, German Collection of Microorganisms and Cell Cultures), from the Japanese Cancer Research Resources Bank (JCRB), from Public Health England, from the European Collection of Authenticated Cell Cultures (ECACC), from Asterand Biosciences, or from the National Cancer Institute (NCI). Cultivated tumour cells were plated at the density as indicated in Table 6 in 96-well multititer plates in 200 microL of their respective growth medium supplemented 10% fetal calf serum. After 24 hours, the cells of one plate (zero-point plate) were stained with crystal violet (see below), while the medium of the other plates was supplemented with the test substances in various concentrations (0 micromol/L, as well as in the range of 0.003-3 micromol/L; the final concentration of the solvent dimethyl sulfoxide was adjusted to 0.1%) using a Tecan HP D300 Digital Dispenser. The cells were incubated for 4 days in the presence of test substances. Cell proliferation was determined by staining the cells with crystal violet: the cells were fixed by adding 20 microL/measuring point of an 11% glutaric aldehyde solution for 15 minutes at room temperature. After three washing cycles of the fixed cells with water, the plates were dried at room temperature. The cells were stained by adding 100 microL/measuring point of a 0.1% crystal violet solution (pH 3.0). After three washing cycles of the stained cells with water, the plates were dried at room temperature. The dye was dissolved by adding 100 microL/measuring point of a 10% acetic acid solution. The extinction was determined by photometry at a wavelength of 595 nm. The change of cell number, in percent, was calculated by normalization of the measured values to the extinction values of the zero-point plate (=0%) and the extinction of the untreated (0 μM) cells (=100%). The T750 values (inhibitory concentration at 50% of maximal effect) were determined by means of a 4 parameter fit.

TABLE 6
Antiproliferative cell line panel data for example no 4 of the present
invention (Result category, IC50 (mean), A: ≤3.0E−08 [mol/l],
B: between 3.0E−08 [mol/l] and 1.0E−07 [mol/l], C: ≥1.0E−07 [mol/l])

| | | | Cell | |
|---|---|---|---|---|
| | | | density, | Result |
| Cell line | Source | Indication | cells/well | Category |
| BT549 | NCI | breast cancer | 4000 | A |
| MCF7 | ATCC HTB-22 | breast cancer | 4000 | C |
| AU565 | ATCC CRL-2351 | breast cancer, HER2 positive | 4000 | A |
| BT474 | ATCC HTB-20 | breast cancer, HER2 positive | 4000 | A |
| EFM192A | DSMZ ACC 258 | breast cancer, HER2 positive | 4000 | B |
| HCC1419 | ATCC CRL-2326 | breast cancer, HER2 positive | 4000 | C |
| HCC1954 | ATCC CRL-2338 | breast cancer, HER2 positive | 4000 | A |
| HCC202 | ATCC CRL-2316 | breast cancer, HER2 positive | 5000 | A |
| JIMT1 | DSMZ ACC 589 | breast cancer, HER2 positive | 4000 | A |
| MDA-MB-361 | ATCC HTB-27 | breast cancer, HER2 positive | 5000 | B |
| SK-BR-3 | ATCC HTB-30 | breast cancer, HER2 positive | 5000 | A |
| CAL-120 | DSMZ ACC 549 | breast cancer, triple-negative | 4000 | A |
| HCC1143 | DSMZ ACC 517 | breast cancer, triple-negative | 4000 | A |
| HCC1599 | ATCC CRL-2331 | breast cancer, triple-negative | 5000 | C |
| HCC1806 | ATCC CRL-2335 | breast cancer, triple-negative | 4000 | C |
| HCC1937 | ATCC CRL-2336 | breast cancer, triple-negative | 5000 | C |
| HCC2157 | ATCC CRL-2340 | breast cancer, triple-negative | 5000 | A |
| HCC70 | ATCC CRL-2315 | breast cancer, triple-negative | 5000 | B |
| MDA-MB-231 | DSMZ ACC 732 | breast cancer, triple-negative | 4000 | A |
| MDA-MB-436 | CLS #300278 | breast cancer, triple-negative | 4000 | A |
| MDA-MB-468 | NCI | breast cancer, triple-negative | 4000 | A |
| SUM149 | Asterand | breast cancer, triple-negative | 4000 | A |
| | Bioscience | | | |
| HeLa | DSMZ ACC 57 | cervical cancer | 3000 | B |
| DLD1 | ATCC CCL-221 | colorectal cancer | 4000 | B |
| HCT116 | DSMZ ACC 581 | colorectal cancer | 3000 | C |
| HT29 | DSMZ ACC 299 | colorectal cancer | 2000 | A |
| Lovo | DSMZ ACC 350 | colorectal cancer | 4000 | B |
| SW480 | DSMZ ACC 313 | colorectal cancer | 3000 | A |
| SW620 | NCI | colorectal cancer | 3000 | A |
| KYSE−410 | DSMZ ACC 381 | estophageal cancer | 4000 | B |
| MKN1 | JRBC CRB0252 | gastric cancer | 4000 | B |
| NCI-N87 | ATCC CRL-5822 | gastric cancer | 4000 | A |
| U251MG | NCI | glioblastoma | 3000 | B |
| HUH- | JRBC 0401 | liver cancer | 4000 | C |
| 6clone5 | | | | |
| HUH-7 | JRBC 0403 | liver cancer | 4000 | A |
| JHH-4 | JRBC 0435 | liver cancer | 4000 | B |
| JHH-7 | JRBC 1031 | liver cancer | 4000 | A |
| PLC/PRF/5 | ATCC CRL-8024 | liver cancer | 4000 | A |
| SK-HEP-1 | DSMZACC 141 | liver cancer | 4000 | A |
| SNU449 | ATCC CRL-2234 | liver cancer | 4000 | B |
| Calu-3 | ATCC HTB-55 | lung cancer, non-small cell | 4000 | B |
| HCC366 | DSMZ ACC 492 | lung cancer, non-small cell | 4000 | B |
| NCI-H1155 | ATCC CRL-5818 | lung cancer, non-small cell | 4000 | B |
| NCI-H1299 | ATCC CRL-5803 | lung cancer, non-small cell | 4000 | B |
| NCI-H1623 | ATCC CRL-5881 | lung cancer, non-small cell | 5000 | B |
| NCI-H1734 | ATCC CRL-5891 | lung cancer, non-small cell | 5000 | A |
| NCI-H1793 | ATCC CRL-5896 | lung cancer, non-small cell | 5000 | B |
| NCI-H1838 | ATCC CRL-5899 | lung cancer, non-small cell | 4000 | A |
| NCI-H1975 | ATCC CRL-5908 | lung cancer, non-small cell | 3000 | B |
| NCI-H2009 | ATCC CRL-5911 | lung cancer, non-small cell | 5000 | B |
| NCI-H2030 | ATCCCRL-5914 | lung cancer, non-small cell | 4000 | A |
| NCI-H23 | ATCC CRL-5800 | lung cancer, non-small cell | 3000 | B |
| NCI-H2405 | ATCC CRL-5944 | lung cancer, non-small cell | 4000 | C |
| NCI-H460 | ATCC HTB-177 | lung cancer, non-small cell | 3000 | C |
| DMS79 | ATCC CRL-2049 | lung cancer, small cell | 5000 | B |
| NCI-H1836 | ATCC HTB-70 | lung cancer, small cell | 5000 | B |
| NCI-H2029 | ATCC CRL-5913 | lung cancer, small cell | 5000 | A |
| NCI-H345 | ATCC HTB-180 | lung cancer, small cell | 5000 | B |
| NCI-H446 | ATCC HTB-171 | lung cancer, small cell | 4000 | B |
| NCI-H526 | ATCC CRL-5811 | lung cancer, small cell | 4000 | B |
| NCI-H69 | ATCC HTB-119 | lung cancer, small cell | 4000 | B |
| NCI-H82 | ATCC HTB-175 | lung cancer, small cell | 4000 | B |
| HT-144 | ATCC HTB-63 | melanoma | 4000 | C |
| COV318 | ECACC | ovarian cancer | 4000 | C |
| | #7071903 | | | |
| COV362 | Public Health | ovarian cancer | 4000 | C |
| | England | | | |
| | #7071910 | | | |
| OVCAR3 | NCI | ovarian cancer | 5000 | B |
| OVCAR8 | NCI | ovarian cancer | 4000 | B |
| SK-OV-3 | ATCC HTB-77 | ovarian cancer | 4000 | C |
| DU145 | DSMZ ACC 261 | prostate cancer | 3000 | C |
| LNCap | DSMZ ACC256 | prostate cancer | 5000 | B |
| PC-3 | DSMZ ACC 465 | prostate cancer | 3000 | B |
| U2-OS | ATCC HTB-96 | sarcoma | 5000 | A |
| A673 | ATCC CRL-1598 | sarcoma | 5000 | A |

### 6. CDK12 Protein Level: Protein Simple (CDK12, CAL-120), Westernblot (CDK9, 12, 13)

CAL-120 human breast cancer cells (DSMZ ACC 459) were seeded at 300,000 cells/well in 6-well plates containing 2 mL of growth medium (DMEM, 10% FCS, glutamine) and incubated for 24 h at 37° C. in an humidified incubator. Test compounds were added at various concentrations, control wells received solvent (DMSO), and the plates were incubated for another 18 h at 37° C. Cells were washed 2 times with PBS and lysed in 75 microL lysis buffer (MSD-Puffer (MSD, #R60TX-2), +1% 505S+PhosSTOP (Roche #04906837001)+complete mini (Roche #04693159001)) by scraping. The lysates were pushed 2 times through Qiashedders followed by centrifugation at 14,000 rpm for 30-50 sec. The supernatant was stored at −20° C. Proteins were separated by applying 0.4 microgramms of protein lysate to Protein Simple 66-440 kDa (Protein Simple #SM-S002) size assay columns on a PEGGY SUE or SALLY SUE equipment according to the supplier's manual. CDK12 and HSP90 (loading control) were detected using anti-human CDK12 antibody (Cell Signaling Technologies (CST) #11793) at 1:25 dilution and anti-human HSP90 antibody (CST #4877) at 1:5,000 dilution. CDK12 and HSP90 peak areas were determined using Protein Simple Compass software. Ratio of CDK12/HSP90 peak areas were calculated for each sample, and 0050 values (degrading concentration to achieve 50% reduction relative to vehicle treated control) were determined by means of a 4 parameter fit.

Westernblot analyses were performed according to standard protocols. 40 microgramms of protein lysates per lane were subjected to polyacrylamide gel electrophoresis using NuPAGE 3-8% tris acetate gels (ThermoFisher) for detection of CDK12 and CDK13 or using NuPAGE 4-12% bis-tris gels (ThermoFisher) for detection of CDK9 followed by protein transfer to nitrocellulose membranes using a BioRad Transblot Turbo equipment. Membranes were probed with rabbit anti CDK12 antibodies (CST #11793), rabbit anti-CDK13 antibodies (Novus #NB 100-68268), anti-CDK9 antibodies (CST #2316), and anti HSP90 (Becton Dickinson #610419) or antd-GAPDH (Zytomed #RGM2-6C5) antibodies for loading control.

TABLE 7
CDK12 protein level in CAL-120 breast cancer cells, DC₅₀-[mol/l]

| | CDK12 protein level in | | CDK12 protein level in |
|---|---|---|---|
| Example | CAL-120 breast ca cells, | Example | CAL-120 breast ca cells, |
| No | DC50-[mol/l] (median) | No | DC50-[mol/l] (median) |
| 1 | 5.74 E−8 | 46 | <3.00 E−8 |
| 4 | 4.76 E−10 | 47 | 5.89 E−8 |
| 14 | 9.38 E−9 | 48 | <3.00 E−8 |
| 15 | 2.98 E−9 | 50 | 3.96 E−8 |
| 16 | 5.81 E−8 | 54 | <3.00 E−8 |
| 20 | 2.60 E−9 | 62 | 1.28 E−7 |
| 21 | >1.00 E−7 | 63 | 4.18 E−8 |
| 22 | 1.18 E−7 | 64 | 8.71 E−8 |
| 23 | <1.00 E−9 | 66 | 4.37 E−8 |
| 24 | 9.33 E−9 | 112 | 5.88 E−08 |
| 25 | <1.00 E−9 | 164 | 1.08 E−09 |
| 27 | 4.46 E−8 | 169 | 1.04 E−09 |
| 28 | 1.44 E−9 | 190 | 3.07 E−10 |
| 29 | 9.15 E−9 | 241 | 7.29 E−09 |
| 31 | 1.09 E−8 | 260 | <1.00 E−9 |

### 7. CDK12 Nuclear Protein Level: Immunofluorescence/High Content Analysis

In the context of the present invention, the term “DC50 CDK12” refers to the DC₅₀ values obtained according to the assay described in this section (7) herein below, i.e. the DC₅₀ values for the degradation of CDK12.

CAL-120 human breast cancer cells (DSMZ ACC 459) are seeded in 1536-well microtiter plates (800 cells per well) containing 50 nanoL of compounds in Dose-Response. Control wells received DMSO or Example 4. Plates are then incubated for 24 h at 37° C. in an humidified incubator and fixed with 4% PFA for 10 min. Then immunofluorescence (IF) against CDK12 (CellSignalling CDK12 Antibody #11973, rabbit, 1:100 dilution) is performed using standard IF protocols. Cells are then stained with Hoechst 33342 (Life Technologies, H-1399, 0.1 microg/ml) and imaged on an automated confocal microscopy system (e.g. Perkin Elmer Opera Phenix). Nuclear and cytoplasmic intensity as well as the nuclear/cytoplasmic intensity ratio is determined by automated image analysis using custom generated scripts (MetaXpress). Data is then transferred to Genedata Screener software, normalized to DMSO and control and DC50 values (degrading concentration to achieve 50% reduction of nuclear CDK12 staining intensity relative to controls) are reported.

TABLE 8
Immunofluorescence CDK12 degradation-
DC₅₀ [mol/L]

| | CDK12 | | CDK12 |
|---|---|---|---|
| | degradation | | degradation |
| | IF, DC50- | | IF, DC50- |
| Example | [mol/l] | Example | [mol/l] |
| No | (median) | No | (median) |
| 1 | 1.11 E−8 | 9 | 8.09 E−8 |
| 2 | 8.46 E−9 | 10 | 6.85 E−9 |
| 3 | 9.45 E−8 | 11 | 1.26 E−8 |
| 4 | 1.07 E−9 | 12 | 4.27 E−8 |
| 5 | 1.14 E−8 | 13 | 4.38 E−8 |
| 6 | 3.32 E−9 | 14 | 1.46 E−8 |
| 7 | 6.36 E−8 | 15 | 5.05 E−9 |
| 8 | 2.07 E−8 | 16 | 4.54 E−9 |
| 17 | 5.98 E−9 | 43 | 9.58 E−9 |
| 18 | 2.98 E−9 | 44 | 1.71 E−9 |
| 19 | 1.09 E−8 | 45 | 6.92 E−8 |
| 20 | 2.57 E−9 | 46 | 1.80 E−8 |
| 21 | 1.86 E−7 | 47 | 5.75 E−9 |
| 22 | 9.90 E−8 | 48 | 2.07 E−8 |
| 23 | 3.45 E−9 | 49 | 4.17 E−8 |
| 24 | 7.78 E−9 | 50 | 1.25 E−8 |
| 25 | 6.74 E−10 | 51 | 7.81 E−9 |
| 26 | 5.84 E−8 | 52 | 2.05 E−8 |
| 27 | 1.28 E−7 | 53 | 4.72 E−8 |
| 28 | 6.50 E−9 | 54 | 3.78 E−8 |
| 29 | 1.18 E−8 | 55 | 3.43 E−9 |
| 30 | 2.57 E−8 | 56 | 1.51 E−8 |
| 31 | 1.97 E−8 | 57 | 1.55 E−9 |
| 32 | 7.21 E−9 | 58 | 2.61 E−8 |
| 33 | 6.78 E−8 | 59 | 5.26 E−9 |
| 34 | 1.27 E−8 | 60 | 9.25 E−9 |
| 35 | 2.73 E−8 | 61 | 5.11 E−9 |
| 36 | 8.65 E−8 | 62 | 1.00 E−8 |
| 37 | 4.28 E−8 | 63 | 2.07 E−8 |
| 38 | 3.72 E−8 | 64 | 1.25 E−8 |
| 39 | 4.14 E−8 | 65 | 4.15 E−9 |
| 40 | 4.58 E−9 | 66 | 1.30 E−7 |
| 41 | 4.06 E−9 | 67 | 3.77 E−9 |
| 42 | 1.25 E−9 | 68 | 1.33 E−8 |
| 69 | 8.45 E−8 | 95 | 3.43 E−8 |
| 70 | 7.90 E−9 | 96 | 5.73 E−9 |
| 71 | 4.34 E−9 | 97 | 3.27 E−10 |
| 72 | 1.00 E−8 | 98 | 3.85 E−8 |
| 73 | 5.15 E−9 | 99 | 6.70 E−9 |
| 74 | 5.74 E−8 | 100 | 1.02 E−8 |
| 75 | 7.38 E−9 | 101 | 5.84 E−10 |
| 76 | 1.21 E−8 | 102 | 1.41 E−9 |
| 77 | 1.16 E−7 | 103 | 5.36 E−9 |
| 78 | 5.15 E−9 | 104 | 1.57 E−8 |
| 79 | 1.23 E−9 | 105 | 2.49 E−9 |
| 80 | 4.28 E−9 | 106 | 1.14 E−8 |
| 81 | 5.31 E−9 | 107 | 5.77 E−8 |
| 82 | 2.23 E−8 | 108 | 5.53 E−9 |
| 83 | 9.37 E−9 | 109 | 1.09 E−9 |
| 84 | 2.12 E−8 | 110 | 1.03 E−9 |
| 85 | 5.57 E−9 | 111 | 1.22 E−9 |
| 86 | 3.95 E−9 | 112 | 5.00 E−08 |
| 87 | 2.16 E−9 | 113 | 8.64 E−09 |
| 88 | 1.63 E−8 | 114 | 3.88 E−08 |
| 89 | 7.30 E−9 | 115 | 4.53 E−08 |
| 90 | 4.05 E−9 | 116 | 4.44 E−09 |
| 91 | 4.28 E−8 | 117 | 3.77 E−09 |
| 92 | 2.36 E−9 | 118 | 4.85 E−09 |
| 93 | 2.33 E−8 | 119 | 1.54 E−09 |
| 94 | 2.50 E−8 | 120 | 5.08 E−09 |
| 121 | 4.35 E−08 | 147 | 1.39 E−08 |
| 122 | 2.47 E−09 | 148 | 1.60 E−08 |
| 123 | 4.37 E−09 | 149 | 1.26 E−08 |
| 124 | 7.68 E−10 | 150 | 1.31 E−09 |
| 125 | 6.30 E−09 | 151 | 2.07 E−09 |
| 126 | 9.06 E−10 | 152 | 1.27 E−08 |
| 127 | 5.10 E−09 | 153 | 2.58 E−09 |
| 128 | 1.37 E−09 | 154 | 5.27 E−09 |
| 129 | 4.98 E−09 | 155 | 4.72 E−08 |
| 130 | 3.31 E−09 | 156 | 1.06 E−09 |
| 131 | 1.22 E−07 | 157 | 6.20 E−10 |
| 132 | 9.22 E−10 | 158 | 5.36 E−10 |
| 133 | 8.46 E−10 | 159 | 5.21 E−10 |
| 134 | 9.91 E−10 | 160 | 1.40 E−09 |
| 135 | 3.69 E−09 | 161 | 8.56 E−10 |
| 136 | 3.61 E−09 | 162 | 4.71 E−10 |
| 137 | 1.08 E−08 | 163 | 3.34 E−10 |
| 138 | 7.60 E−08 | 164 | 9.90 E−10 |
| 139 | 1.10 E−07 | 165 | 4.05 E−10 |
| 140 | 5.83 E−08 | 166 | 3.39 E−09 |
| 141 | 6.11 E−09 | 167 | 1.69 E−08 |
| 142 | 1.62 E−09 | 168 | 5.32 E−10 |
| 143 | 3.09 E−08 | 169 | 1.09 E−09 |
| 144 | 6.32 E−09 | 170 | 5.74 E−10 |
| 145 | 1.68 E−08 | 171 | 2.00 E−09 |
| 146 | 3.82 E−09 | 172 | 1.10 E−09 |
| 173 | 1.74 E−09 | 199 | 5.55 E−09 |
| 174 | 6.45 E−10 | 200 | 1.17 E−07 |
| 175 | 3.22 E−10 | 201 | 6.13 E−08 |
| 176 | 1.53 E−09 | 202 | 3.18 E−08 |
| 177 | 3.57 E−08 | 203 | 6.40 E−10 |
| 178 | 2.55 E−08 | 204 | 6.19 E−10 |
| 179 | 4.59 E−09 | 205 | 5.06 E−10 |
| 180 | 1.59 E−09 | 206 | 4.83 E−09 |
| 181 | 8.78 E−10 | 207 | 1.95 E−09 |
| 182 | 7.27 E−10 | 208 | 8.27 E−10 |
| 183 | 1.65 E−09 | 209 | 4.71 E−10 |
| 184 | 1.75 E−09 | 210 | 1.41 E−09 |
| 185 | 2.49 E−09 | 211 | 2.32 E−09 |
| 186 | 2.26 E−10 | 212 | 1.09 E−09 |
| 187 | 7.07 E−10 | 213 | 2.11 E−09 |
| 188 | 1.26 E−09 | 214 | 3.92 E−09 |
| 189 | 5.57 E−10 | 215 | 7.88 E−09 |
| 190 | 5.53 E−10 | 216 | 3.31 E−09 |
| 191 | 6.06 E−10 | 217 | 8.64 E−09 |
| 192 | 5.60 E−10 | 218 | 1.05 E−08 |
| 193 | 2.68 E−09 | 219 | 1.62 E−08 |
| 194 | 2.30 E−10 | 220 | 4.21 E−09 |
| 195 | 2.18 E−09 | 221 | 4.68 E−09 |
| 196 | 1.90 E−09 | 222 | 4.88 E−08 |
| 197 | 1.40 E−09 | 223 | 5.76 E−08 |
| 198 | 3.49 E−08 | 224 | 3.84 E−08 |
| 225 | 2.21 E−08 | 251 | 1.45 E−10 |
| 226 | 2.76 E−08 | 252 | 1.17 E−09 |
| 227 | 1.68 E−08 | 253 | 8.75 E−10 |
| 228 | 4.71 E−08 | 254 | 8.47 E−09 |
| 229 | 6.38 E−09 | 255 | 1.92 E−08 |
| 230 | 6.60 E−08 | 256 | 3.53 E−09 |
| 231 | 3.39 E−08 | 257 | 3.00 E−09 |
| 232 | >3.60 E−9 | 258 | 3.79 E−09 |
| 233 | 1.03 E−07 | 259 | 7.16 E−09 |
| 234 | 1.03 E−08 | 260 | 1.78 E−09 |
| 235 | 1.04 E−07 | 261 | 1.99 E−08 |
| 236 | 7.18 E−09 | 262 | 1.14 E−08 |
| 237 | 1.28 E−07 | 263 | 2.34 E−08 |
| 238 | 6.49 E−09 | 264 | 3.55 E−09 |
| 239 | 3.47 E−07 | 265 | 7.23 E−09 |
| 240 | 5.56 E−09 | 266 | 5.24 E−08 |
| 241 | 4.34 E−09 | 267 | 3.19 E−08 |
| 242 | 1.07 E−07 | 268 | 1.90 E−09 |
| 243 | 2.67 E−07 | 269 | 3.33 E−09 |
| 244 | 1.01 E−07 | 270 | 1.71 E−09 |
| 245 | 1.49 E−09 | 271 | 1.83 E−08 |
| 246 | 3.00 E−09 | 272 | 7.24 E−09 |
| 247 | 1.38 E−09 | 273 | 1.44 E−09 |
| 248 | 9.46 E−10 | 274 | 8.81 E−10 |
| 249 | 6.48 E−09 | 275 | 1.70 E−09 |
| 250 | 3.16 E−09 | 276 | 3.88 E−08 |
| 277 | 4.48 E−09 | 303 | 1.10 E−08 |
| 278 | 1.18 E−09 | 304 | 7.46 E−08 |
| 279 | 4.72 E−09 | 305 | 1.41 E−08 |
| 280 | 2.14 E−08 | 306 | 1.27 E−08 |
| 281 | 4.03 E−08 | 307 | 8.26 E−09 |
| 282 | 2.79 E−08 | 308 | 4.93 E−09 |
| 283 | 6.97 E−09 | 309 | 8.53 E−09 |
| 284 | 4.85 E−08 | 310 | 1.73 E−08 |
| 285 | 3.23 E−08 | 311 | 6.15 E−09 |
| 286 | 5.38 E−09 | 312 | 3.90 E−09 |
| 287 | 1.01 E−07 | 313 | 2.68 E−08 |
| 288 | 1.16 E−09 | 314 | 8.47 E−09 |
| 289 | 3.13 E−09 | 315 | 3.39 E−08 |
| 290 | 2.15 E−08 | 316 | 5.62 E−09 |
| 291 | 1.92 E−08 | 317 | 5.47 E−09 |
| 292 | 8.78 E−10 | 318 | 3.51 E−09 |
| 293 | 1.71 E−09 | 319 | 3.06 E−09 |
| 294 | 2.73 E−09 | 320 | 4.28 E−09 |
| 295 | 3.52 E−09 | 321 | 6.23 E−09 |
| 296 | 2.55 E−09 | 322 | 4.57 E−09 |
| 297 | 7.59 E−09 | 323 | 1.69 E−08 |
| 298 | 4.22 E−09 | 324 | 1.80 E−08 |
| 299 | 7.77 E−09 | 325 | 1.88 E−09 |
| 300 | 1.79 E−08 | 326 | 4.77 E−09 |
| 301 | 4.37 E−09 | 350 | 6.55 E−09 |
| 327 | 1.00 E−08 | 351 | 2.40 E−08 |
| 328 | 3.95 E−09 | 352 | 5.68 E−09 |
| 329 | 7.38 E−09 | 353 | 1.24 E−08 |
| 330 | 2.89 E−09 | 354 | 7.13 E−08 |
| 331 | 6.00 E−09 | 355 | 2.35 E−08 |
| 332 | 1.07 E−08 | 356 | 6.68 E−09 |
| 333 | 2.31 E−09 | 357 | 3.90 E−09 |
| 334 | 3.35 E−08 | 358 | 5.33 E−09 |
| 335 | 8.34 E−09 | 359 | 4.33 E−09 |
| 336 | 3.08 E−09 | 360 | 6.45 E−09 |
| 337 | 1.85 E−09 | 361 | 6.40 E−09 |
| 338 | 8.94 E−10 | 362 | 7.85 E−09 |
| 339 | 7.59 E−09 | 363 | 1.19 E−08 |
| 340 | 7.80 E−09 | 364 | 3.81 E−09 |
| 341 | 3.55 E−09 | 365 | 2.36 E−08 |
| 342 | 2.12 E−09 | 366 | 9.52 E−09 |
| 343 | 3.56 E−09 | 367 | 1.27 E−08 |
| 344 | 6.69 E−09 | 368 | 9.39 E−09 |
| 345 | 3.98 E−09 | 369 | 2.06 E−08 |
| 346 | 2.35 E−09 | 370 | 2.19 E−09 |
| 347 | 2.01 E−09 | 371 | 4.16 E−09 |
| 348 | 5.30 E−09 | 372 | 2.44 E−09 |
| 349 | 9.89 E−09 | 396 | 1.94 E−08 |
| 373 | 1.12 E−09 | 397 | 2.81 E−08 |
| 374 | 1.81 E−09 | 398 | 6.62 E−08 |
| 375 | 5.81 E−08 | 399 | 6.60 E−08 |
| 376 | 1.24 E−08 | 400 | 6.15 E−09 |
| 377 | 2.02 E−08 | 401 | 3.08 E−08 |
| 378 | 2.91 E−08 | 402 | 7.03 E−09 |
| 379 | 4.35 E−09 | 403 | 4.31 E−08 |
| 380 | 2.32 E−09 | 404 | 1.14 E−07 |
| 381 | 2.22 E−08 | 405 | 1.13 E−07 |
| 382 | 7.97 E−08 | 406 | 1.25 E−07 |
| 383 | 5.79 E−09 | 407 | 2.31 E−09 |
| 384 | 1.10 E−08 | 408 | 5.68 E−08 |
| 385 | 9.13 E−09 | 409 | 2.98 E−08 |
| 386 | 9.83 E−09 | 410 | 1.19 E−09 |
| 387 | 3.83 E−09 | 411 | 4.48 E−08 |
| 388 | 1.04 E−08 | 412 | 7.74 E−08 |
| 389 | 3.35 E−08 | 413 | 1.98 E−08 |
| 390 | 1.92 E−08 | 414 | 3.25 E−08 |
| 391 | 6.98 E−09 | 415 | 1.05 E−07 |
| 392 | 6.41 E−09 | 416 | 2.61 E−08 |
| 393 | 2.23 E−08 | 417 | 2.75 E−09 |
| 394 | 5.65 E−09 | 418 | 3.90 E−08 |
| 395 | 8.00 E−09 | 432 | 9.48 E−10 |
| 419 | 3.87 E−08 | 433 | 4.32 E−09 |
| 420 | 8.06 E−08 | 434 | 5.00 E−09 |
| 421 | 2.16 E−08 | 435 | 5.91 E−10 |
| 422 | 6.81 E−09 | 436 | 1.47 E−08 |
| 423 | 1.78 E−08 | 437 | 1.76 E−09 |
| 424 | 3.81 E−08 | 438 | 1.38 E−07 |
| 425 | 2.51 E−09 | 439 | 4.65 E−09 |
| 426 | 7.51 E−10 | 440 | 6.67 E−09 |
| 427 | 1.47 E−08 | 441 | 6.18 E−09 |
| 428 | 7.03 E−09 | 442 | 4.68 E−09 |
| 429 | 4.32 E−09 | 443 | 4.23 E−09 |
| 430 | 2.53 E−08 | 444 | 7.88 E−09 |
| 431 | 3.39 E−09 | | |

TABLE 9
Inhibition IC50 CDK12 high ATP to Degradation
DC₅₀ CDK12 ratio

| | (IC50 CDK12 | | (IC50 CDK12 |
|---|---|---|---|
| | high ATP) to | | high ATP) to |
| Example | (Degradation DC50 | Example | (Degradation DC50 |
| No | CDK12) ratio | No | CDK12) ratio |
| 1 | 537 | 7 | 161 |
| 2 | >2365 | 8 | >956 |
| 3 | >212 | 9 | >247 |
| 4 | 1628 | 10 | 1277 |
| 5 | 455 | 11 | 251 |
| 6 | 537 | 12 | 399 |
| 13 | >457 | 40 | 532 |
| 14 | >1123 | 41 | >1406 |
| 15 | 1191 | 42 | 324 |
| 16 | 1528 | 43 | >1754 |
| 17 | 957 | 44 | 871 |
| 18 | >5268 | 45 | 32 |
| 19 | 1056 | 46 | 186 |
| 20 | 681 | 47 | 1252 |
| 21 | 52 | 48 | >276 |
| 22 | 40 | 49 | 147 |
| 23 | 452 | 50 | 224 |
| 24 | 564 | 51 | 222 |
| 25 | 1084 | 52 | 80 |
| 26 | 55 | 53 | 43 |
| 27 | 27 | 54 | 150 |
| 28 | 511 | 55 | 224 |
| 29 | 310 | 56 | 352 |
| 30 | 115 | 57 | 669 |
| 31 | 689 | 58 | 246 |
| 32 | 2565 | 59 | 104 |
| 33 | 77 | 60 | 202 |
| 34 | >449 | 61 | >3913 |
| 35 | 81 | 62 | 569 |
| 36 | >231 | 63 | 556 |
| 37 | >467 | 64 | 440 |
| 38 | >153 | 65 | 245 |
| 39 | 113 | 66 | >44 |
| 67 | 727 | 94 | 352 |
| 68 | 415 | 95 | >584 |
| 69 | >68 | 96 | 812 |
| 70 | 79 | 97 | 2411 |
| 71 | 998 | 98 | 28 |
| 72 | 55 | 99 | 229 |
| 73 | 129 | 100 | 122 |
| 74 | >348 | 101 | 3856 |
| 75 | >511 | 102 | 4897 |
| 76 | >440 | 103 | 1914 |
| 77 | 87 | 104 | >1272 |
| 78 | 141 | 105 | 429 |
| 79 | 460 | 106 | >876 |
| 80 | 276 | 107 | >347 |
| 81 | 144 | 108 | >3617 |
| 82 | 41 | 109 | 162 |
| 83 | 111 | 110 | 394 |
| 84 | 98 | 111 | >1337 |
| 85 | 1137 | 112 | 134 |
| 86 | 478 | 113 | 197 |
| 87 | 1196 | 114 | 210 |
| 88 | 84 | 115 | 256 |
| 89 | 1471 | 116 | 1517 |
| 90 | 1092 | 117 | 1278 |
| 91 | >133 | 118 | 1176 |
| 92 | 1378 | 119 | 4303 |
| 93 | 116 | 120 | 2588 |
| 121 | 172 | 148 | 677 |
| 122 | >8081 | 149 | 495 |
| 123 | 2883 | 150 | 2033 |
| 124 | 636 | 151 | 2046 |
| 125 | 1724 | 152 | 236 |
| 126 | 7539 | 153 | 1143 |
| 127 | 1905 | 154 | 1360 |
| 128 | 7939 | 155 | 79 |
| 129 | >3658 | 156 | 8849 |
| 130 | 2547 | 157 | 1089 |
| 131 | 91 | 158 | 8313 |
| 132 | >20812 | 159 | 18457 |
| 133 | 8279 | 160 | 889 |
| 134 | >17850 | 161 | 11233 |
| 135 | 3939 | 162 | 2160 |
| 136 | 1140 | 163 | >17106 |
| 137 | 1196 | 164 | >20194 |
| 138 | >263 | 165 | 25224 |
| 139 | 140 | 166 | 1800 |
| 140 | 140 | 167 | 420 |
| 141 | 214 | 168 | 10872 |
| 142 | 6679 | 169 | 12610 |
| 143 | >646 | 170 | 5303 |
| 144 | 590 | 171 | 3278 |
| 145 | 751 | 172 | 581 |
| 146 | 2320 | 173 | >11498 |
| 147 | 359 | 174 | 1851 |
| 175 | 34272 | 202 | 518 |
| 176 | 1525 | 203 | 13757 |
| 177 | >561 | 204 | 7019 |
| 178 | >783 | 205 | 15749 |
| 179 | >4361 | 206 | 63 |
| 180 | 10852 | 207 | 3942 |
| 181 | 2179 | 208 | 17040 |
| 182 | 2813 | 209 | 14455 |
| 183 | 6199 | 210 | >14143 |
| 184 | 8 | 211 | 1184 |
| 185 | >7872 | 212 | 3798 |
| 186 | 12018 | 213 | 497 |
| 187 | 9192 | 214 | 2062 |
| 188 | 460 | 215 | >2537 |
| 189 | 20 | 216 | >1386 |
| 190 | 11161 | 217 | >2315 |
| 191 | 548 | 218 | >1900 |
| 192 | 15180 | 219 | >1232 |
| 193 | 653 | 220 | 1318 |
| 194 | 16078 | 221 | 1704 |
| 195 | 3781 | 222 | 123 |
| 196 | 8068 | 223 | >287 |
| 197 | 6827 | 224 | 407 |
| 198 | 214 | 225 | 466 |
| 199 | 2661 | 226 | >725 |
| 200 | 37 | 227 | 565 |
| 201 | >326 | 228 | >425 |
| 229 | 862 | 256 | >5662 |
| 230 | >303 | 257 | 39 |
| 231 | >590 | 258 | 1239 |
| 232 | >1467 | 259 | 362 |
| 233 | 73 | 260 | 6819 |
| 234 | 472 | 261 | >942 |
| 235 | >192 | 262 | >500 |
| 236 | >2491 | 263 | 294 |
| 237 | 58 | 264 | 627 |
| 238 | 276 | 265 | >2310 |
| 239 | >58 | 266 | 66 |
| 240 | >3596 | 267 | >627 |
| 241 | 186 | 268 | 1120 |
| 242 | >188 | 269 | 2588 |
| 243 | >75 | 270 | 8023 |
| 244 | >198 | 271 | >1094 |
| 245 | >10636 | 272 | >2761 |
| 246 | >4287 | 273 | 512 |
| 247 | 10678 | 274 | 64 |
| 248 | 9730 | 275 | 57 |
| 249 | >2626 | 276 | >516 |
| 250 | 3560 | 277 | 1071 |
| 251 | 6727 | 278 | 2070 |
| 252 | >17030 | 279 | 2961 |
| 253 | >22856 | 280 | 678 |
| 254 | >2361 | 281 | >419 |
| 255 | >1044 | 282 | >320 |
| 283 | 2630 | 310 | 75 |
| 284 | 113 | 311 | >3251 |
| 285 | 200 | 312 | 2616 |
| 286 | 2107 | 313 | 679 |
| 287 | 106 | 314 | 1171 |
| 288 | 2920 | 315 | >581 |
| 289 | 2240 | 316 | 519 |
| 290 | >920 | 317 | 179 |
| 291 | 554 | 318 | 387 |
| 292 | 758 | 319 | 780 |
| 293 | 3446 | 320 | 130 |
| 294 | 684 | 321 | 67 |
| 295 | 235 | 322 | 266 |
| 296 | 971 | 323 | 169 |
| 297 | 2294 | 324 | 48 |
| 298 | 1968 | 325 | 1890 |
| 299 | 822 | 326 | 198 |
| 300 | >1120 | 327 | 235 |
| 301 | 270 | 328 | 419 |
| 302 | 557 | 329 | 73 |
| 303 | 155 | 330 | 1601 |
| 304 | 167 | 331 | 58 |
| 305 | 765770 | 332 | 18 |
| 306 | 146 | 333 | 61 |
| 307 | 190 | 334 | 60 |
| 308 | 1297 | 335 | 798 |
| 309 | >2346 | 336 | 256 |
| 337 | 4722 | 364 | 1751 |
| 338 | 1320 | 365 | 247 |
| 339 | 204 | 366 | 190 |
| 340 | 79 | 367 | 231 |
| 341 | 624 | 368 | 516 |
| 342 | 3129 | 369 | >970 |
| 343 | 53 | 370 | 77 |
| 344 | 1251 | 371 | 1263 |
| 345 | 166 | 372 | 110 |
| 346 | 5220 | 373 | 2719 |
| 347 | 1086 | 374 | 85 |
| 348 | 546 | 375 | 79 |
| 349 | 877 | 376 | >1615 |
| 350 | 1190 | 377 | 252 |
| 351 | 390 | 378 | 15 |
| 352 | 925 | 379 | 121 |
| 353 | 203 | 380 | 438 |
| 354 | 52 | 381 | 266 |
| 355 | 178 | 382 | 10 |
| 356 | 734 | 383 | 859 |
| 357 | 295 | 384 | 735 |
| 358 | 2182 | 385 | 31 |
| 359 | 483 | 386 | 12 |
| 360 | 622 | 387 | 61 |
| 361 | 224 | 388 | 655 |
| 362 | 1673 | 389 | >597 |
| 363 | 279 | 390 | >1041 |
| 391 | 960 | 418 | >513 |
| 392 | 682 | 419 | >517 |
| 393 | >897 | 420 | >248 |
| 394 | >3303 | 421 | 397 |
| 395 | 800 | 422 | 239 |
| 396 | >1031 | 423 | >720 |
| 397 | >623 | 424 | >524 |
| 398 | 221 | 425 | 852 |
| 399 | >303 | 426 | 32 |
| 400 | >2712 | 427 | 127 |
| 401 | >649 | 428 | >2847 |
| 402 | 32 | 429 | >4626 |
| 403 | >464 | 430 | >789 |
| 404 | >175 | 431 | 829 |
| 405 | >176 | 432 | 2425 |
| 406 | >160 | 433 | 626 |
| 407 | 306 | 434 | >3530 |
| 408 | >352 | 435 | 9674 |
| 409 | >531 | 436 | >1357 |
| 410 | 1336 | 437 | >11369 |
| 411 | >446 | 438 | >57 |
| 412 | >74 | 439 | 850 |
| 413 | >1012 | 440 | >2996 |
| 414 | >176 | 441 | 676 |
| 415 | >191 | 442 | 125 |
| 416 | >765 | 443 | 941 |
| 417 | >2815 | 444 | 1080 |

8. CYCLIN K nuclear protein level: immunofluorescence High Content Analysis

CAL-120 human breast cancer cells (DSMZ ACC 459) are seeded in 1536-well microtiter plates (800 cells per well) containing 50 nanoL of compounds in Dose-Response. Control wells received DMSO or Example 4. Plates are then incubated for 24 h at 37° C. in an humidified incubator and fixed with 4% PFA for 10 min. Then immunofluorescence (IF) against CYCLIN K (ThermoFisher Scientific CONK Antibody #PA5-85020, rabbit, 1:200 dilution) is performed using standard IF protocols. Cells are then stained with Hoechst 33342 (Life Technologies, H-1399, 0.1 microg/ml) and imaged on an automated confocal microscopy system (e.g. Perkin Elmer Opera Phenix). Nuclear and cytoplasmic intensity as well as the nuclear/cytoplasmic intensity ratio is determined by automated image analysis using custom generated scripts (MetaXpress). Data is then transferred to Genedata Screener software, normalized to DMSO and control and DC50 values (degrading concentration to achieve 50% reduction of nuclear CCNK staining intensity relative to controls) are reported.

TABLE 10
Immunofluorescence CYCLIN K degradation-DC₅₀ [mol/L]

| | CYCLIN K | | CYCLIN K |
|---|---|---|---|
| | degradation IF, | | degradation IF, |
| Example | DC50-[mol/l] | Example | DC50-[mol/l] |
| No | (median) | No | (median) |
| 1 | 1.11 E−8 | 20 | 1.35 E−9 |
| 2 | 5.04 E−9 | 23 | 1.02 E−9 |
| 4 | 2.46 E−9 | 24 | 1.67 E−9 |
| 5 | 1.95 E−9 | 25 | 2.63 E−10 |
| 6 | 2.36 E−9 | 27 | 6.63 E−8 |
| 8 | 2.69 E−8 | 28 | 8.88 E−10 |
| 10 | 3.60 E−9 | 29 | 3.36 E−9 |
| 11 | 1.45 E−8 | 30 | 4.10 E−9 |
| 15 | 4.51 E−9 | 39 | 8.13 E−9 |
| 17 | 4.63 E−9 | 40 | 6.11 E−10 |
| 18 | 1.39 E−9 | 42 | 5.87 E−10 |
| 43 | 5.35 E−9 | 67 | 8.14 E−10 |
| 44 | 2.58 E−9 | 68 | 2.85 E−9 |
| 45 | 4.44 E−8 | 70 | 1.17 E−8 |
| 46 | 1.54 E−9 | 71 | 2.77 E−9 |
| 47 | 3.98 E−9 | 72 | 1.14 E−8 |
| 48 | 7.03 E−9 | 73 | 7.35 E−10 |
| 49 | 5.34 E−9 | 75 | 8.70 E−9 |
| 50 | 4.22 E−9 | 76 | 8.06 E−9 |
| 51 | 3.15 E−9 | 78 | 1.61 E−9 |
| 52 | 2.83 E−9 | 79 | 4.69 E−10 |
| 53 | 9.25 E−9 | 80 | 1.34 E−9 |
| 54 | 1.02 E−8 | 81 | 3.87 E−9 |
| 55 | 8.43 E−10 | 82 | 1.42 E−8 |
| 56 | 2.19 E−9 | 83 | 1.78 E−9 |
| 57 | 1.89 E−10 | 84 | 1.18 E−8 |
| 58 | 1.66 E−9 | 85 | 1.20 E−9 |
| 59 | 2.85 E−9 | 86 | 5.76 E−10 |
| 60 | 3.05 E−9 | 87 | 6.67 E−10 |
| 61 | 3.59 E−9 | 88 | 1.16 E−9 |
| 62 | 2.27 E−9 | 89 | 1.43 E−9 |
| 63 | 4.09 E−9 | 90 | 3.57 E−10 |
| 64 | 4.89 E−9 | 91 | 1.96 E−8 |
| 65 | 1.64 E−8 | 92 | 3.81 E−10 |
| 66 | 2.45 E−9 | 93 | 1.73 E−8 |
| 96 | 1.07 E−8 | 127 | 2.77 E−9 |
| 97 | 4.67 E−10 | 128 | 2.16 E−10 |
| 98 | 2.81 E−8 | 129 | 3.39 E−9 |
| 99 | 3.05 E−9 | 130 | 2.01 E−9 |
| 100 | 7.40 E−9 | 132 | 2.22 E−9 |
| 101 | 8.92 E−10 | 133 | 6.33 E−11 |
| 102 | 5.17 E−10 | 134 | 1.72 E−10 |
| 103 | 7.65 E−10 | 135 | 5.54 E−10 |
| 105 | 6.76 E−10 | 136 | 4.64 E−10 |
| 106 | 2.45 E−9 | 137 | 4.68 E−9 |
| 108 | 2.91 E−9 | 141 | 1.74 E−9 |
| 109 | 1.15 E−10 | 142 | 1.63 E−10 |
| 110 | 3.54 E−10 | 146 | 5.60 E−10 |
| 111 | 1.00 E−9 | 148 | 8.06 E−9 |
| 116 | 4.92 E−9 | 150 | 1.48 E−9 |
| 117 | 2.62 E−9 | 151 | 2.45 E−9 |
| 118 | 2.98 E−9 | 152 | 1.11 E−9 |
| 119 | 3.00 E−9 | 153 | 1.42 E−9 |
| 120 | 2.19 E−9 | 154 | 7.13 E−10 |
| 122 | 7.35 E−10 | 156 | 2.49 E−10 |
| 123 | 5.03 E−9 | 157 | 1.19 E−10 |
| 124 | 1.42 E−10 | 158 | 3.67 E−10 |
| 125 | 1.20 E−9 | 159 | 6.63 E−11 |
| 126 | 1.42 E−10 | 160 | 7.43 E−10 |
| 161 | 1.30 E−9 | 189 | 3.18 E−10 |
| 162 | 1.12 E−10 | 190 | 2.68 E−10 |
| 163 | 2.79 E−10 | 191 | 1.87 E−10 |
| 164 | 1.12 E−9 | 192 | 5.89 E−10 |
| 165 | 1.55 E−10 | 193 | 9.58 E−10 |
| 168 | 9.73 E−10 | 194 | 9.54 E−11 |
| 169 | 1.95 E−9 | 195 | 1.29 E−9 |
| 170 | 4.15 E−10 | 196 | 2.29 E−9 |
| 171 | 9.81 E−10 | 197 | 7.63 E−10 |
| 172 | 8.94 E−10 | 199 | 2.32 E−9 |
| 173 | 1.03 E−9 | 203 | 1.19 E−10 |
| 174 | 9.76 E−11 | 204 | 9.67 E−11 |
| 175 | 1.47 E−10 | 205 | 2.79 E−10 |
| 176 | 2.08 E−10 | 206 | 1.31 E−9 |
| 179 | 1.37 E−9 | 207 | 3.19 E−10 |
| 180 | 8.59 E−10 | 208 | 8.71 E−10 |
| 181 | 5.03 E−10 | 209 | 6.19 E−11 |
| 182 | 1.03 E−10 | 210 | 1.99 E−10 |
| 183 | 1.64 E−9 | 211 | 4.78 E−10 |
| 184 | 9.29 E−10 | 212 | 4.70 E−10 |
| 185 | 2.00 E−9 | 213 | 1.55 E−9 |
| 186 | 1.60 E−10 | 214 | 3.47 E−10 |
| 187 | 3.69 E−10 | 215 | 6.49 E−9 |
| 188 | 8.15 E−10 | 216 | 3.54 E−10 |
| 217 | 4.56 E−9 | 252 | 2.42 E−9 |
| 218 | 6.85 E−10 | 253 | 1.82 E−9 |
| 219 | 1.23 E−9 | 254 | 4.17 E−9 |
| 220 | 1.14 E−9 | 256 | 3.26 E−10 |
| 221 | 4.10 E−10 | 257 | 8.64 E−10 |
| 222 | 3.05 E−9 | 260 | 1.52 E−10 |
| 226 | 3.89 E−9 | 261 | 1.23 E−9 |
| 227 | 2.70 E−9 | 262 | 1.94 E−9 |
| 229 | 3.90 E−10 | 263 | 1.82 E−9 |
| 231 | 3.41 E−8 | 264 | 1.80 E−9 |
| 232 | 1.06 E−9 | 265 | 3.14 E−9 |
| 234 | 1.74 E−9 | 268 | 7.56 E−10 |
| 236 | 1.72 E−9 | 269 | 3.24 E−10 |
| 237 | 1.97 E−8 | 270 | 1.90 E−10 |
| 238 | 4.19 E−9 | 271 | 1.21 E−9 |
| 240 | 1.09 E−9 | 272 | 1.38 E−9 |
| 241 | 1.26 E−9 | 273 | 6.60 E−10 |
| 244 | 1.56 E−8 | 274 | 3.21 E−9 |
| 245 | 6.27 E−10 | 275 | 3.03 E−10 |
| 246 | 1.02 E−9 | 278 | 7.28 E−10 |
| 247 | 9.33 E−10 | 279 | 3.78 E−9 |
| 248 | 6.48 E−10 | 281 | 2.08 E−8 |
| 250 | 4.07 E−10 | 283 | 3.67 E−10 |
| 251 | 1.10 E−10 | 285 | 3.46 E−9 |
| 286 | 1.89 E−9 | 311 | 8.92 E−10 |
| 288 | 4.18 E−10 | 312 | 5.18 E−10 |
| 289 | 1.76 E−9 | 313 | 1.57 E−9 |
| 290 | 8.58 E−9 | 314 | 1.11 E−9 |
| 291 | 1.00 E−8 | 315 | 2.15 E−9 |
| 292 | 6.43 E−10 | 317 | 3.47 E−10 |
| 293 | 3.85 E−10 | 318 | 5.16 E−10 |
| 294 | 5.60 E−10 | 319 | 4.92 E−10 |
| 295 | 2.55 E−9 | 321 | 1.36 E−9 |
| 296 | 1.15 E−9 | 325 | 5.30 E−10 |
| 297 | 6.79 E−10 | 326 | 6.54 E−10 |
| 298 | 1.44 E−9 | 327 | 4.93 E−10 |
| 299 | 4.84 E−10 | 330 | 1.60 E−10 |
| 300 | 1.03 E−9 | 331 | 4.40 E−10 |
| 301 | 5.90 E−10 | 333 | 1.13 E−10 |
| 302 | 1.66 E−9 | 334 | 2.06 E−9 |
| 303 | 5.46 E−10 | 336 | 5.20 E−10 |
| 304 | 1.24 E−8 | 337 | 3.69 E−10 |
| 305 | 9.72 E−10 | 338 | 1.70 E−10 |
| 306 | 6.42 E−10 | 340 | 8.27 E−10 |
| 307 | 8.63 E−10 | 341 | 2.90 E−10 |
| 308 | 1.02 E−9 | 342 | ! 5.30 E−11 |
| 309 | 9.14 E−10 | 343 | 9.78 E−11 |
| 310 | 2.90 E−10 | 344 | 3.92 E−10 |
| 345 | 6.98 E−10 | 407 | 6.43 E−10 |
| 346 | 1.49 E−10 | 410 | 4.53 E−10 |
| 347 | 1.01 E−10 | 411 | 1.59 E−8 |
| 348 | 1.17 E−9 | 413 | 6.91 E−9 |
| 349 | 7.84 E−10 | 417 | 1.25 E−9 |
| 353 | 6.44 E−10 | 418 | 2.40 E−9 |
| 354 | 1.65 E−9 | 419 | 9.09 E−9 |
| 355 | 4.81 E−10 | 425 | 5.45 E−10 |
| 356 | 2.01 E−9 | 426 | 5.11 E−10 |
| 357 | 5.52 E−10 | 427 | 9.95 E−9 |
| 360 | 7.09 E−10 | 428 | 6.19 E−9 |
| 370 | 2.58 E−10 | 429 | 3.86 E−9 |
| 371 | 6.52 E−10 | 431 | 1.74 E−9 |
| 372 | 2.27 E−10 | 432 | 3.53 E−10 |
| 373 | 8.05 E−11 | 433 | 4.41 E−10 |
| 374 | 3.45 E−10 | 434 | 2.06 E−9 |
| 379 | 4.04 E−10 | 435 | 2.74 E−10 |
| 380 | 2.20 E−10 | 436 | 1.59 E−8 |
| 384 | 6.62 E−10 | 437 | 2.09 E−9 |
| 392 | 5.19 E−10 | 441 | 2.25 E−9 |
| 395 | 8.84 E−10 | 442 | 3.85 E−10 |
| 398 | 1.04 E−8 | 443 | 2.22 E−9 |
| 402 | 1.78 E−9 | 444 | 5.91 E−9 |

### 9. In Vivo Xenotransplantation Models

The anti-tumor activity of test compound was examined in murine xenotransplantation models of human cancer. For this purpose, mice were implanted subcutaneously or orthotopically with specific human tumor cells. At a mean tumor size of 20-30 mm² animals were randomized into treatment and control groups (n=10 animals/group) and treatment started with vehicle only or Compound (formulation: 80% PEG400/20% Water; application route: p.o./per os, orally; dose/schedule: 5 mg/kg daily (QD), 5 mg/kg twice daily (2QD) for 2 days on/5 days off). The oral application volume was 10 ml/kg. The time interval between two applications per day was 6-7 h. The experiment was ended when the untreated control group had tumors of area s 225 mm². The tumor size and the body weight were determined three times weekly. Changes in the body weight were a measure of treatment-related toxicity (>10%=critical body weight loss and stop of treatment until recovery, >20%=toxic, termination). The tumor area was detected by means of an electronic caliper gauge [length (mm)×width (mm)]. In vivo anti-tumor efficacy is presented as T/C ratio (Treatment/Control) calculated with tumor areas at study end by the formula [(tumor area of treatment group at day x)−(tumor area of treatment group at day before first treatment)]/[(tumor area of control group at day x)−(tumor area of control group at day before first treatment)]. A compound having a T/C below 0.5 is defined as active (effective). Statistical analysis was assessed using SigmaStat software. A one-way analysis of variance was performed and differences to the control were compared by a pair-wise comparison procedure (Dunn's method).

### 10. CYP Inhibition Assay

Use of in vitro assays to evaluate the inhibition potential of new drug candidates towards CYP-mediated metabolism has been shown to be effective as part of a strategy to minimize the chances of drug interactions with co-administered drugs.

The inhibitory potency of the test compound towards 5 human cytochrome P450 isoforms (CYP1A2, 2C8, 2C9, 2D6, and 3A4) was determined during the lead optimization phase. In case of CYP3A4 also time dependent inhibitory potential was tested by applying a 30 min pre-incubation time of the test compound in metabolically active incubation system. Human liver microsomes (pooled, >30 male and female donors) were incubated with individual CYP isoform-selective standard probes (phenacetin, amodiaquine, diclofenac, dextromethorphan and midazolam) in the absence and presence of increasing concentrations of the test compound. Furthermore, the inhibitory potency of standard inhibitors was included as positive controls (fluvoxamine for CYP1A2, montelukast for CYP2C8, sulfaphenazole for CYP2C9, fluoxetine for CYP2D6, ketoconazole for CYP3A4 and mibefradil for CYP3A4-preincubation). Incubation conditions (protein and substrate concentration, incubation time) were optimized with regard to linearity and metabolite turnover. Incubation medium consists of 50 millimol/L potassium phosphate buffer (pH 7.4) containing 1 millimol/L EDTA, NADPH regenerating system (1 millimol/L NADP, 5 millimol/L glucose 6-phosphate, glucose 6-phosphate dehydrogenase (1.5 U/mL). Sequential dilutions and incubations were performed on a Freedom Evo Workstation (Tecan, Crailsheim, FRG) in 96-well plates at 37° C. A final incubation volume of 200 μL was used. Reactions were stopped by addition of 100 μL acetonitrile containing the respective internal standard. After centrifugation the supernatants were analyzed by LC-MS/MS. The LC-MS/MS system for quantification of paracetamol (CYP1A2), desethylamodiaquine (CYP2C8), 4′-hydroxydiclofenac (CYP2C9), dextrorphan (CYP2D6), and 1′-hydroxymidazolam (CYP3A4) comprised a QTRAP 6500@ LC-MS/MS system (Applied Biosystems, MDS Sciex, Ontario, Canada) equipped with an electrospray ionization (ESI) interface (Turboionspray® interface) used to generate positive [M+H]+ ions, an Agilent HP 1290 liquid chromatograph (Agilent Technologies, Waldbronn, Germany) and a HTS PAL autosampler (CTC Analytics, Zwingen, Switzerland).

Data analysis: The CYP-mediated activities in the presence of test compounds (inhibitors) were expressed as percentages of the corresponding no inhibitor control samples. A sigmoid-shaped curve was fitted to the data, and the enzyme inhibition parameter IC50 was calculated using a nonlinear least-squares regression analysis of the plot of percent control activity versus concentration of the test inhibitor.

### 11. Investigation of In Vitro Metabolic Stability in Rat Hepatocytes

Hepatocytes from Han Wistar rats were freshly isolated via a 2-step perfusion method. After perfusion, the liver was carefully removed from the rat: the liver capsule was opened and the hepatocytes were gently shaken out into a Petri dish with ice-cold Williams' medium E (WME). The resulting cell suspension was filtered through sterile gaze in 50 ml falcon tubes and centrifuged at 50×g for 3 min at room temperature. The cell pellet was resuspended in 30 ml WME and centrifuged through a Percoll® gradient for 2 times at 100×g. The hepatocytes were washed again with WME and resuspended in medium containing 5% FCS. Cell viability was determined by trypan blue exclusion.

For the metabolic stability assay liver cells were distributed in WME containing 5% FCS to glass vials at a density of 1.0×10⁶ vital cells/ml. The test compound was added to a final concentration of 1 micromol/L. During incubation, the hepatocyte suspensions were continuously shaken at 580 rpm and aliquots were taken at 2, 8, 16, 30, 45 and 90 min, to which equal volumes of cold methanol were immediately added. Samples were frozen at −20° C. over night, after subsequently centrifuged for 15 minutes at 3000 rpm and the supernatant was analyzed with an Agilent 1200 HPLC-system with LCMS/MS detection.

The half-life of a test compound was determined from the concentration-time plot. From the half-life the intrinsic clearances were calculated. Together with the additional parameters liver blood flow, amount of liver cells in vivo and in vitro. The hepatic in vivo blood clearance (CLblood) and the maximal oral bioavailability (Fmax) was calculated using the following formulae: CL′intrinsic [ml/(min*kg)]=kel [1/min]/((cellno/volume of incubation [ml])*fu,inc)*(cellno/liver weight [g])*(specific liver weight [g liver/kg body weight]); CLblood well-stirred [L/(h*kg)]=(QH [L/(h*kg)]*fu,blood*CL′intrinsic [L/(h*kg)])/(QH [L/(h*kg)]+fu,blood*CL′intrinsic [L/(h*kg)]); Fmax=1-CLblood/QH and using the following parameter values: Liver blood flow (QH)−4.2 L/h/kg rat; specific liver weight−32 g/kg rat body weight; liver cells in vivo-1.1×108 cells/g liver, liver cells in vitro−1.0×106/ml; fu,inc and fu,blood is taken as 1.

### 12. PXR Nuclear Receptor Activation

DPX2 cells (hepatoma cell line stably-cotransfected with a vector for human PXR and a Luciferase reporter gene under the control of two human CYP3A4 promotors, Puracyp, Carlsbad, Calif.) were cultivated according to manufacturer's instructions with following modifications: Cells were seeded in a 384 well plate and cultivated at 37° C./5% CO2 in humidified air. 24 h prior read-out the cells were treated with compound in a ten point serial dilution of −1:3 starting at the highest test concentration of 49.8 micromol/L and ending at 2 nanomol/L. Rifampicin was incubated in the same manner as positive control. In addition, for the normalization of the luminescence signal cells were incubated with Rifampicin at a concentration of 16.7 micromol/L corresponding to 100% activation, as well as DMSO for background luminescence corresponding to 0% activation (n=32 wells each). Cells were lyzed and incubated with the Luciferase substrate ONE-GIo™ Reagent (Promega, Madison Wis., USA) according to manufacturer's instructions and luminescence signal was detected in a plate reader. A concentration-dependent increase of the luciferase activity above 10% of Rifampicin control was classified as PXR transactivation

### 13. In Vivo Pharmacokinetics in Rats

For in vivo pharmacokinetic experiments test compounds were administered to male Wistar rats intravenously at doses of 0.3 to 1 mg/kg and intragastral at doses of 0.5 to 10 mg/kg formulated as solutions using solubilizers such as PEG400 in well-tolerated amounts.

For pharmacokinetics after intravenous administration test compounds were given as i.v. bolus and blood samples were taken at 2 min, 8 min, 15 min, 30 min, 45 min, 1 h, 2 h, 4 h, 6 h, 8 h and 24 h after dosing. Depending on the expected half-life additional samples were taken at later time points (e.g. 48 h, 72 h). For pharmacokinetics after intragastral administration test compounds were given intragastral to fasted rats and blood samples were taken at 5 min, 15 min, 30 min, 45 min, 1 h, 2 h, 4 h, 6 h, 8 h and 24 h after dosing. Depending on the expected half-life additional samples were taken at later time points (e.g. 48 h, 72 h). Blood was collected into Lithium-Heparintubes (Monovetten®, Sarstedt) and centrifuged for 15 min at 3000 rpm. An aliquot of 100 μL from the supernatant (plasma) was taken and precipitated by addition of 400 μL cold acetonitril and frozen at −20° C. over night. Samples were subsequently thawed and centrifuged at 3000 rpm, 4° C. for 20 minutes. Aliquots of the supernatants were taken for analytical testing using an Agilent 1200 HPLC-system with LCMS/MS detection. PK parameters were calculated by non-compartmental analysis using a PK calculation software.

PK parameters derived from concentration-time profiles after i.v.: CLplasma: Total plasma clearance of test compound (in L/kg/h); CLblood: Total blood clearance of test compound: CLplasma*Cp/Cb (in L/kg/h) with Cp/Cb being the ratio of concentrations in plasma and blood. PK parameters calculated from concentration time profiles after i.g.: Cmax: Maximal plasma concentration (in mg/L); Cmaxnorm: Cmax divided by the administered dose (in kg/L); Tmax: Time point at which Cmax was observed (in h). Parameters calculated from both, i.v. and i.g. concentration-time profiles: AUCnorm: Area under the concentration-time curve from t=0 h to infinity (extrapolated) divided by the administered dose (in kg*h/L); AUC(0−tlast)norm: Area under the concentration-time curve from t=0 h to the last time point for which plasma concentrations could be measured divided by the administered dose (in kg*h/L); t1/2: terminal half-life (in h); F: oral bioavailability: AUCnorm after intragastral administration divided by AUCnorm after intravenous administration (in %).

1: A compound of formula (I) 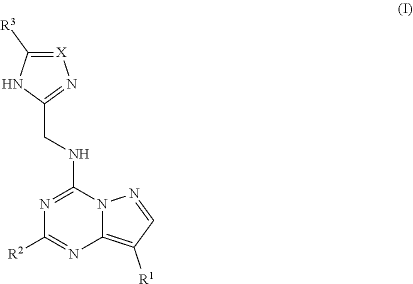
wherein
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a (C₃-C₆-cycloalkyl)-(C₁-C₂-alkyl)-O— group, a (C₃-C₆-hydroxyalkyl)-(C₁-C₂-alkyl)-O— group, a (C₃-C₆-alkoxyalkyl)-(C₁-C₂-alkyl)-O— group, a ((CH₃)₂N)—(C₁-C₂-alkyl)-O— group, a heterocycloalkyl group, a (heterocycloalkyl)-O— group and a —NR^{a}R^{b} group, wherein said heterocycloalkyl group is connected to the rest of the molecule via a carbon atom of said heterocycloalkyl group,
wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
wherein said C₃-C₈-cycloalkyl, phenyl, heteroaryl or heterocycloalkyl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group and a R⁵R⁶N— group,
wherein at least one of R^{a} or R^{b} is not a hydrogen atom;
or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂- and —S(═O)(═NH)—,
and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
wherein said heterocycloalkyl, heterocycloalkenyl or heteroaryl group contains one or two heteroatoms independently selected from nitrogen, oxygen and sulfur,
and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
or a tautomer, or an N-oxide, or a salt thereof, or a salt of a tautomer, or a salt of an N-oxide, or a mixture of same.

2: The compound according to claim 1, wherein
R¹ is selected from a halogen atom, a C₁-C₄-alkyl group, a C₁-C₃-haloalkyl group, a C₃-C₅-cycloalkyl group, a cyano group, a phenyl group, a heterocycloalkyl group and a heteroaryl group,
wherein said C₁-C₄-alkyl, C₃-C₅-cycloalkyl, phenyl, heterocycloalkyl or heteroaryl group is each optionally substituted with one or more substituents independently selected from a halogen atom, a C₁-C₆-alkyl group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a (C₃-C₆-cycloalkyl)-(C₁-C₂-alkyl)-O— group, a (C₃-C₆-hydroxyalkyl)-(C₁-C₂-alkyl)-O— group, a (C₃-C₆-alkoxyalkyl)-(C₁-C₂-alkyl)-O— group, a ((CH₃)₂N)—(C₁-C₂-alkyl)-O— group, a heterocycloalkyl group, a (heterocycloalkyl)-O— group and a —NR^{a}R^{b} group, wherein said heterocycloalkyl group is connected to the rest of the molecule via a carbon atom of said heterocycloalkyl group,
wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group and a R⁵R⁶N— group,
wherein at least one of R^{a} or R^{b} is not a hydrogen atom;
or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
wherein said heterocycloalkyl, heterocycloalkenyl or heteroaryl group contains one or two heteroatoms independently selected from nitrogen, oxygen and sulfur,
and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
or a tautomer, or an N-oxide, or a salt thereof, or a salt of a tautomer, or a salt of an N-oxide, or a mixture of same.

3: The compound according to claim 1,
wherein
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group, a (C₃-C₆-cycloalkyl)-(C₁-C₂-alkyl)-O— group, a (C₃-C₆-hydroxyalkyl)-(C₁-C₂-alkyl)-O— group, a (C₃-C₆-alkoxyalkyl)-(C₁-C₂-alkyl)-O— group, a ((CH₃)₂N)—(C₁-C₂-alkyl)-O— group, a heterocycloalkyl group, a (heterocycloalkyl)-O— group and a —NR^{a}R^{b} group, wherein said heterocycloalkyl group is connected to the rest of the molecule via a carbon atom of said heterocycloalkyl group,
wherein R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group and a R⁵R⁶N— group,
wherein at least one of R^{a} or R^{b} is not a hydrogen atom;
or R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
wherein said heterocycloalkyl, heterocycloalkenyl or heteroaryl group contains one or two heteroatoms independently selected from nitrogen, oxygen and sulfur,
and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
or a tautomer, or an N-oxide, or a salt thereof, or a salt of a tautomer, or a salt of an N-oxide, or a mixture of same.

4: The compound according to claim 1,
wherein
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
wherein said heterocycloalkyl, heterocycloalkenyl or heteroaryl group contains one or two heteroatoms independently selected from nitrogen, oxygen and sulfur,
and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
or a tautomer, or an N-oxide, or a salt thereof, or a salt of a tautomer, or a salt of an N-oxide, or a mixture of same.

5: The compound according to claim 1,
wherein
R¹ is selected from a halogen atom, a C₁-C₃-alkyl group, a C₁-C₃-haloalkyl group, a C₃-C₆-cycloalkyl group and a C₃-C₆-halocycloalkyl group;
R² is a —NR^{a}R^{b} group,
wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
wherein said heterocycloalkyl, heterocycloalkenyl or heteroaryl group contains one or two heteroatoms independently selected from nitrogen, oxygen and sulfur,
and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
or a tautomer, or an N-oxide, or a salt thereof, or a salt of a tautomer, or a salt of an N-oxide, or a mixture of same.

6: The compound according to claim 1,
wherein
R¹ is selected from a C₁-C₆-alkyl group, a C₁-C₆-haloalkyl group, a C₃-C₈-cycloalkyl group, a C₃-C₈-halocycloalkyl group and a (C₃-C₈-cycloalkyl)-(C₁-C₆-alkyl)- group;
R² is a —NR^{a}R^{b} group,
wherein R^{a} and R^{b} together with the nitrogen atom to which they are attached form a morpholine ring, a 5- to 11-membered nitrogen-containing bicyclic heterocycloalkyl group, a 7- to 9-membered nitrogen-containing bridged compound or a 7- to 12-membered nitrogen-containing spiro compound,
said 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
said morpholine ring, 5- to 11-membered nitrogen-containing bicyclic heterocycloalkyl group, 7- to 9-membered nitrogen-containing bridged compound or 7- to 12-membered nitrogen-containing spiro compound each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
wherein said heterocycloalkyl, heterocycloalkenyl or heteroaryl group contains one or two heteroatoms independently selected from nitrogen, oxygen and sulfur,
and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
or a tautomer, or an N-oxide, or a salt thereof, or a salt of a tautomer, or a salt of an N-oxide, or a mixture of same.

7: The compound according to claim 1,
wherein
R¹ is selected from a C₁-C₆-alkyl group, a C₁-C₆-haloalkyl group, a C₃-C₈-cycloalkyl group, a C₃-C₈-halocycloalkyl group and a (C₃-C₈-cycloalkyl)-(C₁-C₆-alkyl)- group;
R² is a —NR^{a}R^{b} group,
wherein R^{a} and R^{b} together with the nitrogen atom to which they are attached form a morpholine ring,
said morpholine ring optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
wherein said heterocycloalkyl, heterocycloalkenyl or heteroaryl group contains one or two heteroatoms independently selected from nitrogen, oxygen and sulfur,
and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
or a tautomer, or an N-oxide, or a salt thereof, or a salt of a tautomer, or a salt of an N-oxide, or a mixture of same.

8: The compound according to claim 1,
wherein
R¹ is selected from a C₁-C₆-alkyl group, a C₁-C₆-haloalkyl group, a C₃-C₈-cycloalkyl group, a C₃-C₈-halocycloalkyl group and a (C₃-C₈-cycloalkyl)-(C₁-C₆-alkyl)- group;
R² is a —NR^{a}R^{b} group,
and R^{a} and R^{b} together with the nitrogen atom to which they are attached form an azetidine ring or a morpholine ring,
said azetidine ring or morpholine ring each optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a phenyl group and a heteroaryl group,
wherein said phenyl or heteroaryl group is each optionally substituted with one or more substituents independently selected from a halogen atom, a cyano group, a hydroxy group, a C₁-C₆-alkyl group, a C₁-C₆-haloalkyl group, a C₁-C₆-alkoxy group, a C₁-C₆-haloalkoxy group,
and wherein, when substituted, said phenyl group is preferably substituted in one or more of the ortho- and/or meta-positions with respect to the point of attachment of said phenyl group to the rest of the molecule;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
wherein said heterocycloalkyl, heterocycloalkenyl or heteroaryl group contains one or two heteroatoms independently selected from nitrogen, oxygen and sulfur,
and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
or a tautomer, or an N-oxide, or a salt thereof, or a salt of a tautomer, or a salt of an N-oxide, or a mixture of same.

9: The compound according to claim 1,
wherein
R¹ is selected from a halogen atom, a C₁-C₃-alkyl group, a C₁-C₃-haloalkyl group, a C₃-C₆-cycloalkyl group and a C₃-C₆-halocycloalkyl group;
R² is selected from a C₁-C₆-alkoxy group, a C₁-C₆-haloalkoxy group, a C₃-C₈-cycloalkoxy group, a (C₃-C₆-cycloalkyl)-(C₁-C₂-alkyl)-O— group, a (C₃-C₆-hydroxyalkyl)-(C₁-C₂-alkyl)-O— group, a (C₃-C₆-alkoxyalkyl)-(C₁-C₂-alkyl)-O— group, a ((CH₃)₂N)—(C₁-C₂-alkyl)-O— group and a (heterocycloalkyl)-O— group,
wherein said heterocycloalkyl group is connected to the rest of the molecule via a carbon atom of said heterocycloalkyl group;
X is a CR⁴ group;
and R³ and R⁴, together with the carbon atoms to which they are attached form a 6-membered cycloalkenyl group, a phenyl group or a 6-membered heteroaryl group,
wherein said heteroaryl group contains one or two heteroatoms independently selected from nitrogen, oxygen and sulfur,
and wherein said cycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
R⁸ is selected from a hydrogen atom, a C₁-C₆-alkyl group, a C₃-C₆-cycloalkyl group and a C₁-C₆-haloalkyl group;
or a tautomer, or an N-oxide, or a salt thereof, or a salt of a tautomer, or a salt of an N-oxide, or a mixture of same.

10: The compound according to claim 1,
wherein
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group;
R² is a —NR^{a}R^{b} group,
wherein R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound containing one, two or three further heteroatoms independently selected from, oxygen and sulfur or one group selected from —S(═O)—, —S(═O)₂— and —S(═O)(═NH)—,
and/or optionally being substituted one, two or three times, each substituent independently selected from a halogen atom or a group selected from, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a R⁵R⁶N— group and oxo;
X is selected from a nitrogen atom and a CR⁴ group;
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group;
R⁴ is selected from a hydrogen atom, a C₁-C₃-alkyl group and a C₁-C₃-haloalkyl group;
or, where X is a CR⁴ group, R³ and R⁴, together with the carbon atoms to which they are attached form a 5- to 7-membered cycloalkyl, cycloalkenyl, heterocycloalkyl, heterocycloalkenyl, phenyl or heteroaryl group,
wherein said heterocycloalkyl, heterocycloalkenyl or heteroaryl group contains one or two heteroatoms independently selected from nitrogen, oxygen and sulfur,
and wherein said cycloalkyl, cycloalkenyl, heterocycloalkyl, heterocycloalkenyl, phenyl or heteroaryl group is each optionally substituted one, two or three times, each substituent independently selected from a halogen atom, a cyano group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₅-cycloalkyl group, a C₃-C₅-cycloalkoxy group, a R⁵R⁶N— group, a (R⁵R⁶N)—(C₁-C₆-alkyl)- group, and a R⁷OOC— group;
R⁵ and R⁶ are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a formyl (HCO—) group, an acetyl (H₃CCO—) group, a heterocycloalkyl group, a heteroaryl group and a phenyl group;
R⁷ is selected from a hydrogen atom and a C₁-C₃-alkyl group;
or a tautomer, or an N-oxide, or a salt thereof, or a salt of a tautomer, or a salt of an N-oxide, or a mixture of same.

11: The compound of according to claim 1, wherein the compound is selected from the group consisting of:
N-[(1H-benzimidazol-2-yl)methyl]-8-bromo-2-(morpholin-4-yl)pyrazolo[1,5-a][1,3,5]triazin-4-amine,
8-bromo-N-[(7-methyl-3H-imidazo[4,5-b]pyridin-2-yl)methyl]-2-(morpholin-4-yl)pyrazolo[1,5-a][1,3,5]triazin-4-amine,
8-bromo-2-(morpholin-4-yl)-N-[(5-phenyl-4H-1,2,4-triazol-3-yl)methyl]pyrazolo[1,5-a][1,3,5]triazin-4-amine,
8-bromo-N-{[5-(3-fluorophenyl)-4H-1,2,4-triazol-3-yl]methyl}-2-(morpholin-4-yl)pyrazolo[1,5-a][1,3,5]triazin-4-amine,
N-[(1H-benzimidazol-2-yl)methyl]-8-cyclopropyl-2-(morpholin-4-yl)pyrazolo[1,5-a][1,3,5]triazin-4-amine,
8-bromo-N-[(4-fluoro-1H-benzimidazol-2-yl)methyl]-2-(morpholin-4-yl)pyrazolo[1,5-a][1,3,5]triazin-4-amine,
8-bromo-2-[(2R,6S)-2,6-dimethylmorpholin-4-yl]-N-[(4-fluoro-1H-benzimidazol-2-yl)methyl]pyrazolo[1,5-a][1,3,5]triazin-4-amine,
8-bromo-N-[(4-fluoro-1H-benzimidazol-2-yl)methyl]-2-(4-methylpiperazin-1-yl)pyrazolo[1,5-a][1,3,5]triazin-4-amine,
8-bromo-N-[(4-methyl-1H-benzimidazol-2-yl)methyl]-2-(morpholin-4-yl)pyrazolo[1,5-a][1,3,5]triazin-4-amine,
8-bromo-N-[(5-methoxy-1H-benzimidazol-2-yl)methyl]-2-(morpholin-4-yl)pyrazolo[1,5-a][1,3,5]triazin-4-amine,
8-cyclopropyl-N-[(4,5-difluoro-1H-benzimidazol-2-yl)methyl]-2-(morpholin-4-yl)pyrazolo[1,5-a][1,3,5]triazin-4-amine,
8-cyclopropyl-N-[(4,5-difluoro-1H-benzimidazol-2-yl)methyl]-2-(piperazin-1-yl)pyrazolo[1,5-a][1,3,5]triazin-4-amine,
N-[(1H-benzimidazol-2-yl)methyl]-8-bromo-2-(4,7-diazaspiro[2.5]octan-7-yl)pyrazolo[1,5-a][1,3,5]triazin-4-amine,
8-cyclopropyl-2-[(3R,5S)-3,5-dimethylpiperazin-1-yl]-N-[(4-fluoro-1H-benzimidazol-2-yl)methyl]pyrazolo[1,5-a][1,3,5]triazin-4-amine,
8-cyclopropyl-N-[(4-fluoro-1H-benzimidazol-2-yl)methyl]-2-(morpholin-4-yl)pyrazolo[1,5-a][1,3,5]triazin-4-amine,
8-cyclopropyl-N-[(4-methoxy-1H-benzimidazol-2-yl)methyl]-2-(4-methylpiperazin-1-yl)pyrazolo[1,5-a][1,3,5]triazin-4-amine,
8-cyclopropyl-N-[(4-methoxy-1H-benzimidazol-2-yl)methyl]-2-morpholino-pyrazolo[1,5-a][1,3,5]triazin-4-amine,
8-cyclopropyl-N-[(4-methoxy-1H-benzimidazol-2-yl)methyl]-2-[(4aS,7aS)-octahydro-6H-pyrrolo[3,4-b]pyridin-6-yl]pyrazolo[1,5-a][1,3,5]triazin-4-amine,
N-(1H-benzimidazol-2-ylmethyl)-8-bromo-2-(2,5-diazabicyclo[2.2.2]octan-2-yl)pyrazolo[1,5-a][1,3,5]triazin-4-amine,
1-[(4-{[(1H-benzimidazol-2-yl)methyl]amino}-8-bromopyrazolo[1,5-a][1,3,5]triazin-2-yl)(methyl)amino]-2-methylpropan-2-ol,
N-[(1H-benzimidazol-2-yl)methyl]-2-(morpholin-4-yl)-8-(trifluoromethyl)pyrazolo[1,5-a][1,3,5]triazin-4-amine,
2-(4-aminopiperidin-1-yl)-N-[(1H-benzimidazol-2-yl)methyl]-8-bromopyrazolo[1,5-a][1,3,5]triazin-4-amine,
N-[(5-methoxy-1H-benzimidazol-2-yl)methyl]-2-(morpholin-4-yl)-8-(trifluoromethyl)pyrazolo[1,5-a][1,3,5]triazin-4-amine,
N-(1H-benzimidazol-2-ylmethyl)-8-bromo-2-[(2R or S)-2,4-dimethylpiperazin-1-yl]pyrazolo[1,5-a][1,3,5]triazin-4-amine,
N-[(1H-benzimidazol-2-yl)methyl]-8-bromo-2-[4-(methylamino)piperidin-1-yl]pyrazolo[1,5-a][1,3,5]triazin-4-amine,
N-[(5-fluoro-1H-benzimidazol-2-yl)methyl]-2-(morpholin-4-yl)-8-(trifluoromethyl)pyrazolo[1,5-a][1,3,5]triazin-4-amine,
N-[(4-fluoro-1H-benzimidazol-2-yl)methyl]-2-(morpholin-4-yl)-8-(trifluoromethyl)pyrazolo[1,5-a][1,3,5]triazin-4-amine,
N-[(5-chloro-1H-benzimidazol-2-yl)methyl]-2-(morpholin-4-yl)-8-(trifluoromethyl)pyrazolo[1,5-a][1,3,5]triazin-4-amine,
2-[(1R,5S,6s)-6-amino-3-azabicyclo[3.1.1]heptan-3-yl]-N-[(1H-benzimidazol-2-yl)methyl]-8-bromopyrazolo[1,5-a][1,3,5]triazin-4-amine,
1-[(8-cyclopropyl-4-{[(4,5-difluoro-1H-benzimidazol-2-yl)methyl]amino}pyrazolo[1,5-a][1,3,5]triazin-2-yl)(methyl)amino]-2-methylpropan-2-ol,
N-[(4-fluoro-1H-benzimidazol-2-yl)methyl]-2-(4-methylpiperazin-1-yl)-8-(trifluoromethyl)pyrazolo[1,5-a][1,3,5]triazin-4-amine,
8-cyclopropyl-2-[(1R,5S)-3,8-diazabicyclo[3.2.1]octan-3-yl]-N-[(4,5-difluoro-1H-benzimidazol-2-yl)methyl]pyrazolo[1,5-a][1,3,5]triazin-4-amine,
1-[(8-cyclopropyl-4-{[(4-fluoro-1H-benzimidazol-2-yl)methyl]amino}pyrazolo[1,5-a][1,3,5]triazin-2-yl)(methyl)amino]-2-methylpropan-2-ol,
8-cyclopropyl-2-[(1R,5S)-3,8-diazabicyclo[3.2.1]octan-3-yl]-N-[(4-fluoro-1H-benzimidazol-2-yl)methyl]pyrazolo[1,5-a][1,3,5]triazin-4-amine,
N-[(5-chloro-1H-benzimidazol-2-yl)methyl]-2-(4-methylpiperazin-1-yl)-8-(trifluoromethyl)pyrazolo[1,5-a][1,3,5]triazin-4-amine,
2-(3,8-diazabicyclo[3.2.1]octan-3-yl)-N-[(4,5-difluoro-1H-benzimidazol-2-yl)methyl]-8-(trifluoromethyl)pyrazolo[1,5-a][1,3,5]triazin-4-amine,
N-[(5,6-dichloro-1H-benzimidazol-2-yl)methyl]-2-(morpholin-4-yl)-8-(trifluoromethyl)pyrazolo[1,5-a][1,3,5]triazin-4-amine,
N-[(5,6-difluoro-1H-benzimidazol-2-yl)methyl]-2-(morpholin-4-yl)-8-(trifluoromethyl)pyrazolo[1,5-a][1,3,5]triazin-4-amine,
N-[(4,5-difluoro-1H-benzimidazol-2-yl)methyl]-2-(8-methyl-3,8-diazabicyclo[3.2.1]octan-3-yl)-8-(trifluoromethyl)pyrazolo[1,5-a][1,3,5]triazin-4-amine,
N-[(4-fluoro-1H-benzimidazol-2-yl)methyl]-2-(8-methyl-3,8-diazabicyclo[3.2.1]octan-3-yl)-8-(trifluoromethyl)pyrazolo[1,5-a][1,3,5]triazin-4-amine,
N-{[5-(3-fluorophenyl)-4H-1,2,4-triazol-3-yl]methyl}-2-(morpholin-4-yl)-8-(trifluoromethyl)pyrazolo[1,5-a][1,3,5]triazin-4-amine,
N-{[5-(3,5-difluorophenyl)-4H-1,2,4-triazol-3-yl]methyl}-2-(morpholin-4-yl)-8-(trifluoromethyl)pyrazolo[1,5-a][1,3,5]triazin-4-amine,
2-[(2S)-2,4-dimethylpiperazin-1-yl]-N-[(4-fluoro-1H-benzimidazol-2-yl)methyl]-8-(trifluoromethyl)pyrazolo[1,5-a][1,3,5]triazin-4-amine,
N-[(4,5-difluoro-1H-benzimidazol-2-yl)methyl]-8-methyl-2-(morpholin-4-yl)pyrazolo[1,5-a][1,3,5]triazin-4-amine,
1-{[4-{[(4,5-difluoro-1H-benzimidazol-2-yl)methyl]amino}-8-(trifluoromethyl)pyrazolo[1,5-a][1,3,5]triazin-2-yl](methyl)amino}-2-methylpropan-2-ol,
2-(1,4-diazabicyclo[3.2.2]nonan-4-yl)-N-[(4,5-difluoro-1H-benzimidazol-2-yl)methyl]-8-(trifluoromethyl)pyrazolo[1,5-a][1,3,5]triazin-4-amine,
2-(1,4-diazabicyclo[3.2.2]nonan-4-yl)-N-[(4-fluoro-1H-benzimidazol-2-yl)methyl]-8-(trifluoromethyl)pyrazolo[1,5-a][1,3,5]triazin-4-amine,
N-[(4,5-difluoro-1H-benzimidazol-2-yl)methyl]-2-(morpholin-4-yl)-8-(trifluoromethyl)pyrazolo [1,5-a][1,3,5]triazin-4-amine,
N-[(1H-benzimidazol-2-yl)methyl]-8-ethyl-2-(morpholin-4-yl)pyrazolo[1,5-a][1,3,5]triazin-4-amine and
N-[(4,5-difluoro-1H-benzimidazol-2-yl)methyl]-8-ethyl-2-(morpholin-4-yl)pyrazolo[1,5-a][1,3,5]triazin-4-amine.

12: The compound according to claim 1, wherein the compound has a ratio (IC50 CDK12 hATP)/(DC50 CDK12) which is equal or greater than 20 and/or a (DC50 CDK12) value which is equal or lower than 200 nM.

13: The compound according to claim 1, wherein the compound has a ratio (IC50 CDK12 hATP)/(DC50 CDK12) which is equal or greater than 5 and/or a (DC50 CDK12) value which is equal or lower than 200 nM.

14. (canceled)

15: A method for treatment or prophylaxis of a disease, comprising administering to a subject in need thereof an effective amount of a compound of formula (I), or a stereoisomer, a tautomer, an N-oxide, a hydrate, a solvate, or a salt thereof, particularly a pharmaceutically acceptable salt thereof, or a mixture of same, according to claim 1.

16. (canceled)

17. (canceled)

18: The method of claim 15, wherein the disease is a hyperproliferative disease.

19. The method of claim 18, wherein the hyperproliferative disease is cancer.

20: The method of claim 18, wherein the hyperproliferative disease is selected from the group consisting of lung cancer, breast cancer, liver cancer, colorectal cancer, gastric cancer, prostate cancer and leukemia.

21: A pharmaceutical composition comprising a compound of formula (I) or a stereoisomer, a tautomer, an N-oxide, a hydrate, a solvate, or a salt thereof, particularly a pharmaceutically acceptable salt thereof, or a mixture of same, according to claim 1, and a pharmaceutically acceptable carrier.

22: A method for the treatment or prophylaxis of a hyperproliferative disease, comprising administering to a subject in need thereof an effective amount of a pharmaceutical composition according to claim 21.

23: A pharmaceutical combination comprising:
One or more first active ingredients selected from a compound according to claim 1, and
One or more second active ingredients selected from chemotherapeutic anti-cancer agents.

24: A process for the preparation of a compound of formula (I), according to claim 1, wherein the compound of formula (I) is a compound of formula (Ia), wherein R² is a —NR^{a}R^{b} group, a C₁-C₆-alkoxy group or a C₃-C₈-cycloalkoxy group and wherein R¹, R³, R^{a}, R^{b} and X are as defined in claim 1, 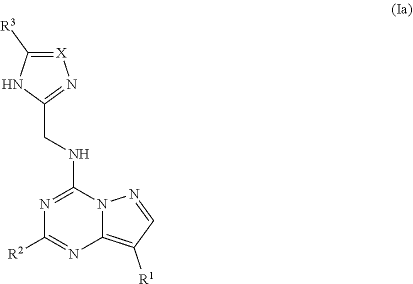
said process comprising reacting an intermediate compound of general formula (II) 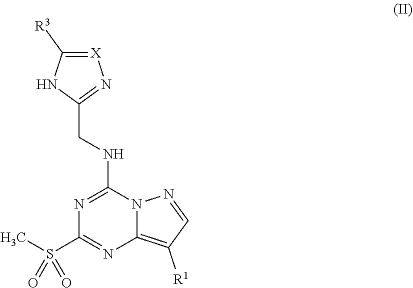
wherein R¹, R³, and X are as defined in claim 1,
with a compound of formula (IIIa)
R²—H (IIIa),
wherein R² is a —NR^{a}R^{b} group, a C₁-C₆-alkoxy group or a C₃-C₈-cycloalkoxy group and wherein R^{a} and R^{b} are as defined in claim 1,
thereby giving a compound of formula (Ia).

25: A compound of general formula (II) 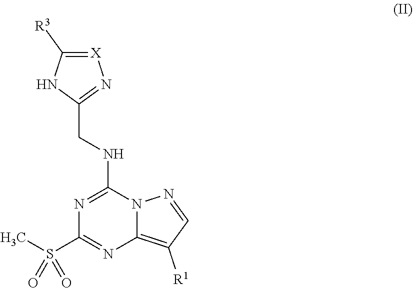
wherein
R¹ is selected from a halogen atom, a C₁-C₆-alkyl group, a C₁-C₆-haloalkyl group, a C₃-C₈-cycloalkyl group, a (C₃-C₈-cycloalkyl)-(C₁-C₆-alkyl)- group, a cyano group, a phenyl group, a heterocycloalkyl group and a heteroaryl group,
wherein said C₁-C₆-alkyl, C₃-C₈-cycloalkyl, phenyl, heterocycloalkyl or heteroaryl group is each optionally substituted with one or more substituents independently selected from a halogen atom, a cyano group, a C₁-C₆-alkyl group, a C₁-C₆-alkoxy group, a C₁-C₆-haloalkyl group, a C₁-C₆-haloalkoxy group, a C₃-C₈-cycloalkyl group, a C₃-C₈-cycloalkoxy group and a R⁵R⁶N— group:
X is selected from a nitrogen atom and a CR⁴ group; and
R³ is selected from a C₃-C₈-cycloalkyl group, a heterocycloalkyl group, a phenyl group and a heteroaryl group,
wherein said heterocycloalkyl, phenyl or heteroaryl group is each optionally substituted with one or more substituents independently selected from a halogen atom, a cyano group, a hydroxy group, a C₁-C₆-alkyl group, a C₁-C₆-hydroxyalkyl group, a C₁-C₆-haloalkyl group, a C₁-C₆-alkoxy group, a C₁-C₆-haloalkoxy group, a C₃-C₈-cycloalkyl group, a R⁵R⁶N— group, and a R⁷OOC— group.

26: Use of a compound of general formula (II) 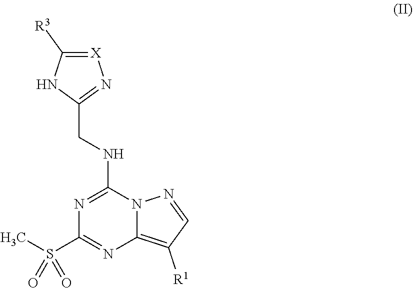
according to claim 25,
for the preparation of a compound of formula (Ia), 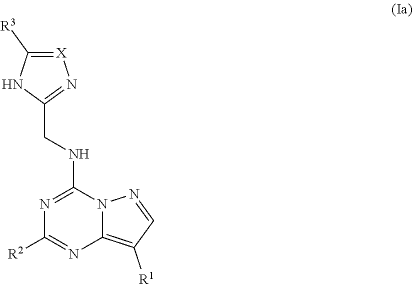
wherein R¹, R³, and X are as defined in claim 25,
R² is a —NR^{a}R^{b} group, a C₁-C₆-alkoxy group or a C₃-C₈-cycloalkoxy group:
R^{a} and R^{b} are each independently selected from a hydrogen atom, a C₁-C₆-alkyl group, a C₃-C₈-cycloalkyl group, a C₁-C₆-haloalkyl group, a (C₃-C₈-cycloalkyl)-(C₁-C₆-alkyl)- group, a C₁-C₆-hydroxyalkyl group, a (C₁-C₆-alkoxy)-(C₁-C₆-alkyl)- group, a heterocycloalkyl group, a heteroaryl group and a phenyl group,
wherein said C₃-C₈-cycloalkyl, phenyl, heteroaryl or heterocycloalkyl group is each optionally substituted with one or more substituents independently selected from a halogen atom, a hydroxy group, a cyano group, a C₁-C₃-alkyl group, a C₁-C₃-hydroxyalkyl group, a C₁-C₃-haloalkyl group, a C₁-C₃-alkoxy group, a C₁-C₃-haloalkoxy group and a R⁵R⁶N— group,
wherein at least one of R^{a} or R^{b} is not a hydrogen atom:
or
R^{a} and R^{b} together with the nitrogen atom to which they are attached form a 4- to 9-membered nitrogen-containing monocyclic or 5- to 11-membered nitrogen-containing bicyclic heterocycloalkyl group, a 7- to 9-membered nitrogen-containing bridged compound or a 7- to 12-membered nitrogen-containing spiro compound,
said 4- to 9-membered nitrogen-containing monocyclic or 5- to 11-membered nitrogen-containing bicyclic heterocycloalkyl group, 7- to 9-membered nitrogen-containing bridged compound or 7- to 12-membered nitrogen-containing spiro compound each optionally containing one, two or three further heteroatoms independently selected from nitrogen, oxygen and sulfur or one group selected from —NR⁸—, —S(═O)—, —S(═O)₂— and —S(═O)(═NH)—,
and/or optionally being substituted one, two or three times, each substituent independently selected from a halogen atom or a group selected from cyano, hydroxy, a C₁-C₂-alkyl group, a C₁-hydroxyalkyl group, a C₁-C₂-haloalkyl group, a C₁-C₂-alkoxy group, a C₃-C₄-cycloalkyl group, a (C₁-C₂-alkyl)OOC— group, a R⁵R⁶N— group and oxo.

27: The method of claim 18, wherein the hyperproliferative disorder is breast cancer, liver cancer, lung cancer, ovarian cancer, endometrial cancer, cervical cancer, colorectal cancer, gastric cancer, esophageal cancer, bladder cancer, prostate cancer, Ewing sarcoma, glioblastoma or acute myeloid leukemia.


SEQUENCE LISTING (7 sequences)

SEQ ID NO: 1 - protein, 425 aa, Homo sapiens
  His-CDK12
MTSHHHHHHSSMGSRTSLYKKAGSDYDIPTTENLYFQGQPYKKRPKICCPRYGERRQTESDWGKRCVDKFDIIGIIGEGTYGQVYKAKDKDTGELVALKKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLADFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNS

SEQ ID NO: 2 - protein, 425 aa, Homo sapiens
  His-CDK12-DN
MTSHHHHHHSSMGSRTSLYKKAGSDYDIPTTENLYFQGQPYKKRPKICCPRYGERRQTESDWGKRCVDKFDIIGIIGEGTYGQVYKAKDKDTGELVALAKVRLDNEKEGFPITAIREIKILRQLIHRSVVNMKEIVTDKQDALDFKKDKGAFYLVFEYMDHDLMGLLESGLVHFSEDHIKSFMKQLMEGLEYCHKKNFLHRDIKCSNILLNNSGQIKLANFGLARLYNSEESRPYTNKVITLWYRPPELLLGEERYTPAIDVWSCGCILGELFTKKPIFQANLELAQLELISRLCGSPCPAVWPDVIKLPYFNTMKPKKQYRRRLREEFSFIPSAALDLLDHMLTLDPSKRCTAEQTLQSDFLKDVELSKMAPPDLPHWQDCHELWSKKRRRQRQSGVVVEEPPPSKTSRKETTSGTSTEPVKNS

SEQ ID NO: 3 - protein, 425 aa, Homo sapiens
  His-CDK13
MTSHHHHHHSSMGSRTSLYKKAGSDYDIPTTENLYFQGQLHSKRRPKICGPRYGETKEKDIDWGKRCVDKFDIIGIIGEGTYGQVYKARDKDTGEMVALKKVRLDNEKEGFPITAIREIKILRQLTHQSIINMKEIVTDKEDALDFKKDKGAFYLVFEYMDHDLMGLLESGLVHFNENHIKSFMRQLMEGLDYCHKKNFLHRDIKCSNILLNNRGQIKLADFGLARLYSSEESRPYTNKVITLWYRPPELLLGEERYTPAIDVWSCGCILGELFTKKPIFQANQELAQLELISRICGSPCPAVWPDVIKLPYFNTMKPKKQYRRKLREEFVFIPAAALDLFDYMLALDPSKRCTAEQALQCEFLRDVEPSKMPPPDLPLWQDCHELWSKKRRRQKQMGMTDDVSTIKAPRKDLSLGLDDSRTNTP

SEQ ID NO: 4 - protein, 425 aa, Homo sapiens
  His-CDK13-DN
MTSHHHHHHSSMGSRTSLYKKAGSDYDIPTTENLYFQGQLHSKRRPKICGPRYGETKEKDIDWGKRCVDKFDIIGIIGEGTYGQVYKARDKDTGEMVALAKVRLDNEKEGFPITAIREIKILRQLTHQSIINMKEIVTDKEDALDFKKDKGAFYLVFEYMDHDLMGLLESGLVHFNENHIKSFMRQLMEGLDYCHKKNFLHRDIKCSNILLNNRGQIKLANFGLARLYSSEESRPYTNKVITLWYRPPELLLGEERYTPAIDVWSCGCILGELFTKKPIFQANQELAQLELISRICGSPCPAVWPDVIKLPYFNTMKPKKQYRRKLREEFVFIPAAALDLFDYMLALDPSKRCTAEQALQCEFLRDVEPSKMPPPDLPLWQDCHELWSKKRRRQKQMGMTDDVSTIKAPRKDLSLGLDDSRTNTP

SEQ ID NO: 5 - protein, 338 aa, Homo sapiens
  His-CycK
MTSHHHHHHSSMGSRTSLYKKAGSDYDIPTTENLYFQGMKENKENSSPSVTSANLDHTKPCWYWDKKDLAHTPSQLEGLDPATEARYRREGARFIFDVGTRLGLHYDTLATGIIYFHRFYMFHSFKQFPRYVTGACCLFLAGKVEETPKKCKDIIKTARSLLNDVQFGQFGDDPKEEVMVLERILLQTIKFDLQVEHPYQFLLKYAKQLKGDKNKIQKLVQMAWTFVNDSLCTTLSLQWEPEIIAVAVMYLAGRLCKFEIQEWTSKPMYRRWWEQFVQDVPVDVLEDICHQILDLYSQGKQQMPHHTPHQLQQPPSLQPTPQVPQVQQSQPSQSSEPS

SEQ ID NO: 6 - protein, 368 aa, Homo sapiens
  CAK1
MKLDSIDITHCQLVKSTRTARIYRSDTYAIKCLALDFDIPPHNAKFEVSILNKLGNKCKHILPLLESKATDNNDLLLLFPFEEMNLYEFMQMHYKRDRRKKNPYYDLLNPSIPIVADPPVQKYTNQLDVNRYSLSFFRQMVEGIAFLHENKIIHRDIKPQNIMLTNNTSTVSPKLYIIDFGISYDMANNSQTSAEPMDSKVTDISTGIYKAPEVLFGVKCYDGGVDVWSLLIIISQWFQRETSRMGHVPAMIDDGSDDMNSDGSDFRLICSIFEKLGIPSIQKWEEVAQHGSVDAFVGMFGADGDGKYVLDQEKDVQISIVERNMPRLDEIADVKVKQKFINCILGMVSFSPNERWSCQRILQELEKP

SEQ ID NO: 7 - protein, 18 aa, Homo sapiens
  biotinylated peptide fragment
KFELLPTPPLSPSRRSGL